SEQUENCE LISTING (21 sequences)

SEQ ID NO: 1 - protein, 19 aa, synthetic construct
  REGION 1..19: note = Angiopep-2
TFFYGGSRGKRNNFKTEEY

SEQ ID NO: 2 - protein, 39 aa, synthetic construct
  REGION 1..39: note = ApoB (3371-3409)
SSVIDALQYKLEGTTRLTRKRGLKLATALSLSNKFVEGS

SEQ ID NO: 3 - protein, 9 aa, synthetic construct
  REGION 1..9: note = ApoE (159-167)2
  REPEAT 1..9: note = Wherein the sequence can be repeated twice
LRKLRKRLL

SEQ ID NO: 4 - protein, 8 aa, synthetic construct
  REGION 1..8: note = Peptide-22
  MOD_RES 1: note = ACETYLATION
  DISULFID 1: note = join (1,8)
  MOD_RES 1: note = AMIDATION
CMPRLRGC

SEQ ID NO: 5 - protein, 12 aa, synthetic construct
  REGION 1..12: note = THR
THRPPMWSPVWP

SEQ ID NO: 6 - protein, 12 aa, synthetic construct
  REGION 1..12: note = THR retro-enantio
  SITE 1..12: note = D-amino acid
PWVPSWMPPRHT

SEQ ID NO: 7 - protein, 9 aa, synthetic construct
  REGION 1..9: note = CRT
  DISULFID 1: note = join (1,9)
CRTIGPSVC

SEQ ID NO: 8 - protein, 30 aa, synthetic construct
  REGION 1..30: note = Leptin30
YQQILTSMPSRNVIQISNDLENLRDLLHVL

SEQ ID NO: 9 - protein, 29 aa, synthetic construct
  REGION 1..29: note = RVG29
YTIWMPENPRPGTPCDIFTNSRGKRASNG

SEQ ID NO: 10 - protein, 16 aa, synthetic construct
  REGION 1..16: note = DCDX
  SITE 2..5: note = D-amino acid
  SITE 7..16: note = D amino acid
GREIRTGRAERWSEKF

SEQ ID NO: 11 - protein, 18 aa, synthetic construct
  REGION 1..18: note = Apamin
  DISULFID 1: note = Join (1,10)
  DISULFID 2: note = Join (2,14)
  MOD_RES 17: note = AMIDATION
CNCKAPETALCARRCQQH

SEQ ID NO: 12 - protein, 8 aa, synthetic construct
  REGION 1..8: note = MiniAp-4
  SITE 1: note = mod_res - N and C terminus linked by diaminopropyl
KAPETALD

SEQ ID NO: 13 - skipped

SEQ ID NO: 14 - protein, 13 aa, synthetic construct
  REGION 1..13: note = G23
HLNILSTLWKYRC

SEQ ID NO: 15 - protein, 7 aa, synthetic construct
  REGION 1..7: note = g7
  CARBOHYD 7: note = O-linked beta-GLC
  MOD_RES 7: note = AMIDATION
  SITE 3: note = D-amino Acid
GFTGFLS

SEQ ID NO: 16 - protein, 12 aa, synthetic construct
  REGION 1..12: note = TGN
TGNYKALHPHNG

SEQ ID NO: 17 - protein, 11 aa, synthetic construct
  REGION 1..11: note = TAT (47-57)
YGRKKRRQRRR

SEQ ID NO: 18 - protein, 18 aa, synthetic construct
  REGION 1..18: note = SynB1
RGGRLSYSRRRFSTSTGR

SEQ ID NO: 19 - skipped

SEQ ID NO: 20 - protein, 4 aa, synthetic construct
  REGION 1..4: note = PhPro
  REGION 1..4: note = mod_res - phenylated
PPPP

SEQ ID NO: 21 - protein, 18 aa, synthetic construct
  REGION 1..18: note = ApoE (159-167)2
LRKLRKRLLLRKLRKRLL

(19) United States
(12) Patent Application Publication
(10) Pub. No.: US 2025/0129067 A1
(43) Pub. Date: Apr. 24, 2025

(54) PLATELET-DERIVED GROWTH FACTOR RECEPTOR (PDGFR) ALPHA INHIBITORS AND USES THEREOF

(71) Applicant: Progentos Therapeutics, Inc., Winchester, MA (US)

(72) Inventors: Sanjay Shivayogi MAGAVI, Winchester, MA (US); Daniel J. PARKS, Winchester, MA (US); Bradley Dean TAIT, Winchester, MA (US); Jinhyung CHO, Winchester, MA (US); Rajiv AGRAWAL, Winchester, MA (US); Patricia R. SHAW, Winchester, MA (US)

(73) Assignee: Progentos Therapeutics, Inc., Winchester, MA (US)

(21) Appl. No.: 18/707,909

(22) PCT Filed: Nov. 8, 2022
(86) PCT No.: PCT/US2022/079480

§ 371 Date: May 7, 2024

Related U.S. Application Data
(60) Provisional application No. 63/277,145, filed on Nov. 8, 2021.
(60) Provisional application No. 63/378,431, filed on Oct. 5, 2022.

(51) Int. Cl. C07D 471/04; A61K 31/437; A61K 31/4545; A61K 31/496; A61K 31/5025; A61K 31/506; A61K 31/519; A61K 31/5377; A61K 31/551; A61K 45/06; C07D 487/04; C07D 519/00; C07K 16/28; G01N 33/50

(52) U.S. Cl. CPC C07D 471/04; A61K 31/437; A61K 31/4545; A61K 31/496; A61K 31/5025; A61K 31/506; A61K 31/519; A61K 31/5377; A61K 31/551; A61K 45/06; C07D 487/04; C07D 519/00; C07K 16/2863; G01N 33/5088

(57) ABSTRACT

The present disclosure provides compounds that can specifically target a PDGFRα, and thereby, reduce and/or inhibit the activation of the receptor (“PDGFRα inhibitor”). The present disclosure also provides methods of treating a demyelinating disease using the disclosed PDGFRα inhibitors.

### CROSS-REFERENCE TO RELATED APPLICATIONS

This PCT application claims the priority benefit of U.S. Provisional Application No. 63/277,145, filed Nov. 8, 2021, and U.S. Provisional Application No. 63/378,431, filed Oct. 5, 2022, both of which are incorporated herein by reference in their entirety.

### REFERENCE TO SEQUENCE LISTING SUBMITTED ELECTRONICALLY VIA EFS-WEB

The content of the electronically submitted sequence listing (Name: 4552_009PC02_Seqlisting_ST26.xml, Size: 30,923 bytes; and Date of Creation: Nov. 8, 2022) submitted in this application is incorporated herein by reference in its entirety.

### FIELD OF THE DISCLOSURE

The present disclosure relates to compounds that can promote remyelination. Specifically, the disclosure relates to PDGFRα inhibitors and the use of such inhibitors to treat diseases associated with impaired myelination (e.g., multiple sclerosis).

### BACKGROUND OF THE DISCLOSURE

Proper myelination by oligodendrocytes is essential for central nervous system (CNS) development and function. Gacem et al., Life 11 (4): 327 (April 2021). Myelin maintains axon potential conduction velocity of neural signals and provides metabolic support to axons, supporting their survival. During normal development, oligodendrocyte progenitor cells (OPCs) undergo morphological and molecular changes as they differentiate into oligodendrocytes that can myelinate axons. Accordingly, any abnormal developmental processes or pathogenic immune activation and failure of oligodendrocytes to myelinate axons or loss of myelin can lead to neurodegenerative diseases such as multiple sclerosis.

Multiple sclerosis (MS) is generally characterized by inflammation and demyelination of neuronal axons. Patients with early MS often suffer from isolated immune attacks with effective remyelination and recovery between attacks. However, as the disease progresses, MS patients have a reduced ability to effectively remyelinate resulting in permanent neurological disability. Current treatments for MS and other neurodegenerative diseases largely focus on reducing the immune attack on myelin but do not restore myelin on the damaged neuronal axons.

Therefore, there remains a need for a new and more effective treatment of such demyelinating diseases, particularly those that can induce the remyelination of demyelinated, vulnerable neuronal axons.

### BRIEF SUMMARY OF THE DISCLOSURE

The present disclosure describes the discovery that oligodendrocyte progenitor cells (OPCs) can be induced to differentiate into oligodendrocytes and remyelinate demyelinated axons by inhibiting PDGFRα. Furthermore, the present disclosure describes the discovery that such compounds can promote remyelination. Compounds and methods of the present disclosure are therefore useful for treating diseases associated with demyelination (e.g., hypomyelination).

Provided herein is a compound of Formula I:

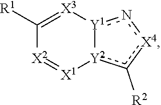

or a pharmaceutically acceptable salt or solvate thereof, wherein:
[symbol] indicates a single bond or a double bond such that all valences are satisfied;
X¹, X², X³, and X⁴ are selected from N and CR^{a}, with the proviso that not more than two of X¹, X², X³, and X⁴ are N;
one of Y¹ and Y² is N and the other of Y¹ and Y² is C;
each R^{a} is independently selected from H, halo, C₁-C₄alkyl, and C₁-C₄alkoxy;
R¹ is selected from C₁-C₄alkyl, C₃-C₈cycloalkyl, 3-8 membered heterocyclyl, heteroaryl, aryl, and C₁-C₈alkoxy, all of which can be optionally substituted with one, two, three, four, five, or six substituents selected from halo, hydroxy, oxo, C₁-C₄alkyl, aminoC₁-C₄alkyl, hydroxyC₁-C₄alkyl, C₁-C₄alkoxy, C₁-C₄alkoxyC₁-C₄alkyl, 3-8 membered heterocyclyl, and 3-8 membered heterocyclylC₁-C₄alkyl, with the proviso that the number of substituents does not exceed the number of substitutable positions;
R² is selected from cycloalkyl, cycloalkenyl, alkyl, oxoalkylamino, aminoalkylamino, amino, heterocyclyl, heteroaryl, aminoheterocyclyl, heterocyclylamino, and aminoalkylamino, all of which can be optionally substituted with one, two, three, four, or five substituents selected from D, halo, hydroxy, oxo, and C₁-C₄alkyl;
R² is substituted by one, two, or three R³,
R³ is selected from aryl, heteroaryl, —C(O)R³¹, —C(O)OR³¹, —C(O)NR³¹R³², —S(O)₂NR³¹R³², —S(O)(NR³³)R³¹, —S(O)(NR³³)NR³¹R³², —C(S)NR³¹R³², C₃-C₈cycloalkyl, 3-8 membered heterocyclyl, and C₁-C₄alkyl, all of which can be optionally substituted with one, two, three, four, or five R³⁰;
each R³⁰ is independently selected from D, halo, aryl, —OR³⁰⁰, —NR³⁰⁰R³⁰³, —S(O)ᵣR³⁰⁰, —C(O)R³⁰⁰, —C(═CR³⁴R³⁵)R³⁰⁰, and

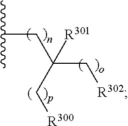

r is selected from 0, 1, and 2;
each R³⁰⁰ is independently selected from C₁-C₆alkyl, C₃-C₇cycloalkyl, aryl, heteroaryl, 3-8 membered heterocyclyl, and 3-8 membered heterocyclylaryl, all of which can be optionally substituted with one, two, three, four, or five substituents selected from D, halo, hydroxy, amino, alkylamino, cyano, C₁-C₄alkyl, C₁-C₄haloalkyl, and C₁-C₄alkoxy;
each R³⁰¹ is independently selected from H, halo, and C₁-C₄alkyl;
each R³⁰² is independently selected from H, F, hydroxyl, amino, alkylamino, oxo, and C₁-C₄alkyoxy;
each R³⁰³ is independently selected from H and C₁-C₄alkyl;
n, o, and p are each independently selected from 0, 1, 2, 3, and 4;
each R³¹ is independently selected from C₁-C₈alkyl, arylC₁-C₄alkyl, heteroarylC₁-C₄alkyl, heterocyclyl, heterocyclylC₁-C₄alkyl, cycloalkyl, and cycloalkylC₁-C₄alkyl, all of which can be optionally substituted with one, two, three, four, or five substituents selected from D, halo, cyano, hydroxy, amino, —OCF₃, C₁-C₄alkyl, C₁-C₄haloalkyl, C₁-C₄alkoxy, hydroxyC₁-C₄alkyl, —S(O)₂NR³⁰⁴R³⁰⁵, —C(O)OR³⁰⁴R³⁰⁵, —C(O)NR³⁰⁴R³⁰⁵, and —NR³⁰⁴C(O)R³⁰⁵;
each R³⁰⁴ and R³⁰⁵ is independently selected from H and C₁-C₄alkyl;
each R³² is independently selected from H and C₁-C₄alkyl; or
R³¹ and R³² together with the atom to which they are connected to form a 5-8 membered heterocycyl, optionally substituted with one, two, three, four, or five substituents selected from D, halo, cyano, C₁-C₄alkyl, C₁-C₄haloalkyl, and —C(O)NR³⁴R³⁵; and
each R³⁴ and R³⁵ is independently selected from H, C₁-C₄alkyl, and C₁-C₄haloalkyl; and
each R³³ is independently selected from H, C₁-C₄alkyl, C₁-C₄haloalkyl, and —C(O)R³⁴; or R³¹ and R³³ together with the atoms to which they are connected form a 4-8 membered heterocycyl.

In some aspects, Y¹ is N and Y² is C.

In some aspects, Y¹ is C and Y² is N.

In some aspects, X¹ is N, X² is CR^{a}, X³ is CR^{a}, and X⁴ is CR^{a}.

In some aspects, X¹ is CR^{a}, X² is N, X³ is CR^{a}, and X⁴ is CR^{a}.

In some aspects, X¹ is CR^{a}, X² is CR^{a}, X³ is N, and X⁴ is CR^{a}.

In some aspects, X¹ is CR^{a}, X² is CR^{a}, X³ is CR^{a}, and X⁴ is N.

In some aspects, X¹ is CR^{a}, X² is CR^{a}, X³ is CR^{a}, and X⁴ is CR^{a}.

In some aspects, R¹ is 5- or 6-membered heteroaryl.

In some aspects, R¹ is an optionally substituted pyrazolyl.

In some aspects, R¹ is selected from:

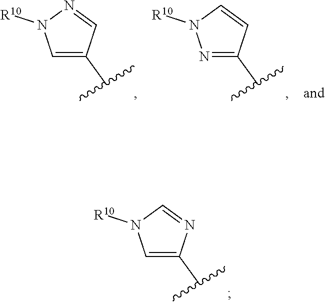

and

R¹⁰ is selected from H, C₁-C₄alkyl, C₁-C₄alkoxy, aminoC₁-C₄alkyl, hydroxyC₁-C₄alkyl, and C₁-C₄alkylsulfonyl, wherein the C₁-C₄alkyl, C₁-C₄alkoxy, aminoC₁-C₄alkyl, hydroxyC₁-C₄alkyl, and C₁-C₄alkylsulfonyl can be optionally substituted by one or more substituents selected from hydroxyl, C₁-C₄alkoxy, NR^{10a}R^{10b}, halo, and deuterium, wherein R^{10a} and R^{10b} are selected from hydrogen and C₁-C₄alkyl, or wherein R^{10a} and R^{10b} taken together with the nitrogen atom to which they are attached form a 4- to 8-membered ring.

In some aspects, R¹ is

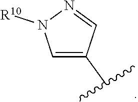

In some aspects, R¹⁰ is CH₃.

In some aspects, R² is heterocyclyl.

In some aspects, R² is selected from:

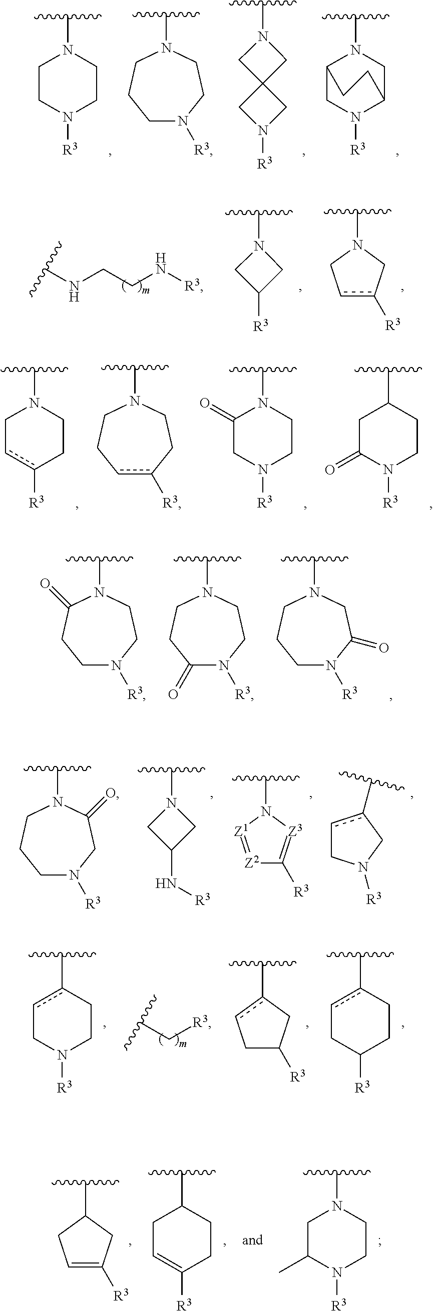

[symbol] indicates a single bond or a double bond such that all valences are satisfied;
m is selected from 0, 1, 2, 3, 4, 5, and 6; and
Z¹, Z², and Z³ are selected from N and CR^{a}.

In some aspects, R² is selected from:

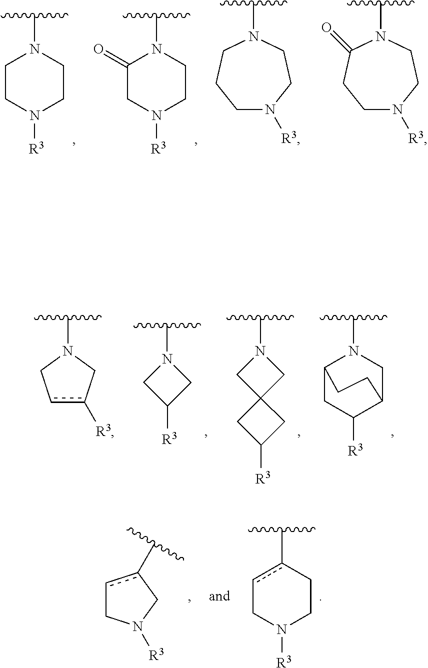

In some aspects, the compound, or a pharmaceutically acceptable salt or solvate thereof, has Formula Ia:

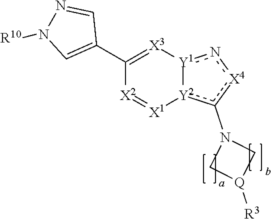

wherein a and b are each independently selected from 1, 2, and 3; and
Q is selected from —CH— and —N—, with the proviso that if Q is —N—, a and b are not 1.

In some aspects, the compound, or a pharmaceutically acceptable salt or solvate thereof, is a compound of Formula II:

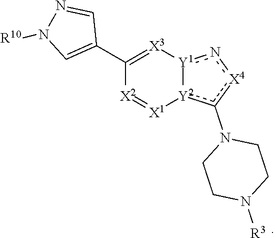

In some aspects, the compound, or a pharmaceutically acceptable salt or solvate thereof, has Formula IIa:

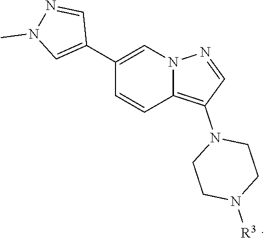

In some aspects, R³ is heteroaryl optionally substituted with one, two, three, four, or five R³⁰.

In some aspects, R³ is selected from:

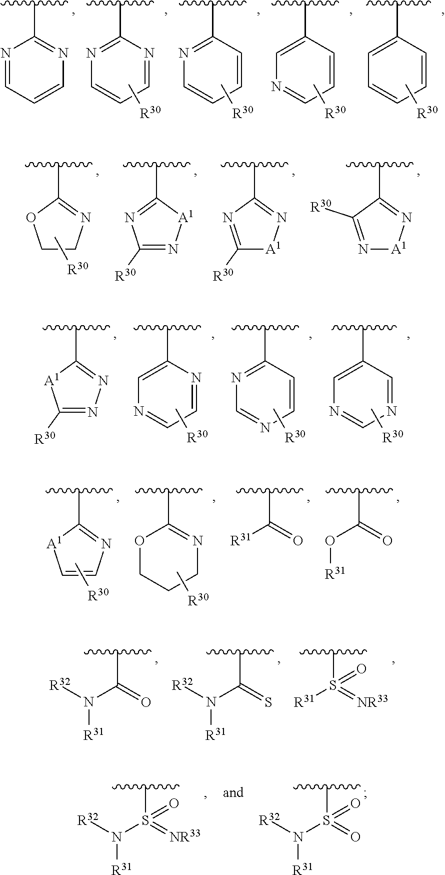

wherein A¹ is selected from O, S, and N.

In some aspects, R³ is selected from:

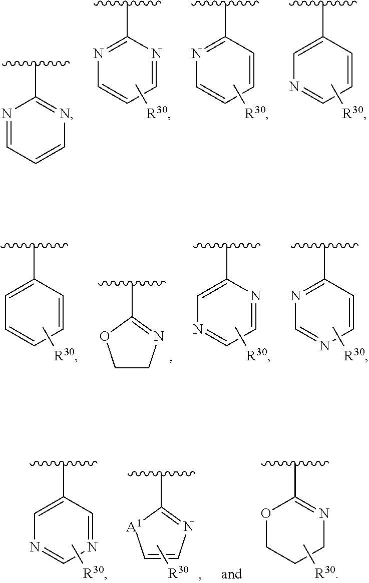

In some aspects, R³⁰ is:

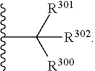

In some aspects, R³⁰⁰ is selected from:

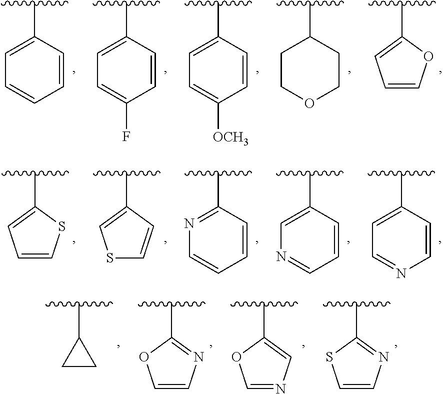

### and H.

In some aspects, R³⁰⁰ is selected from:

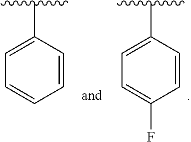 [SMILES: *c1ccccc1 ; *c1ccc(F)cc1]

In some aspects, the compound, or a pharmaceutically acceptable salt or solvate thereof, has Formula III:

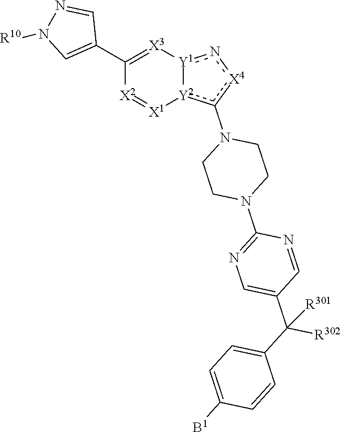

wherein B¹ is H, Cl, F, or —CF₃.

In some aspects, R³⁰¹ is H and R³⁰² is —OH or CH₃.

In some aspects, R³⁰¹ is H and R³⁰² is —OH.

In some aspects, R³ is selected from:

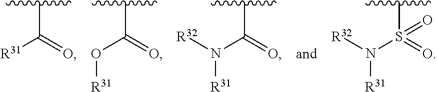

In some aspects, R³ is selected from:

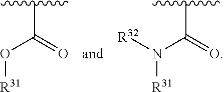

In some aspects, the compound, or a pharmaceutically acceptable salt or solvate thereof, has Formula IVa:

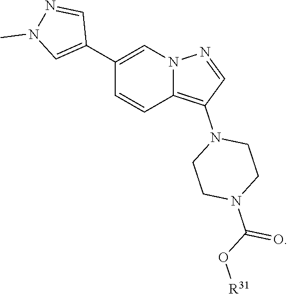

In some aspects, R³¹ is selected from:

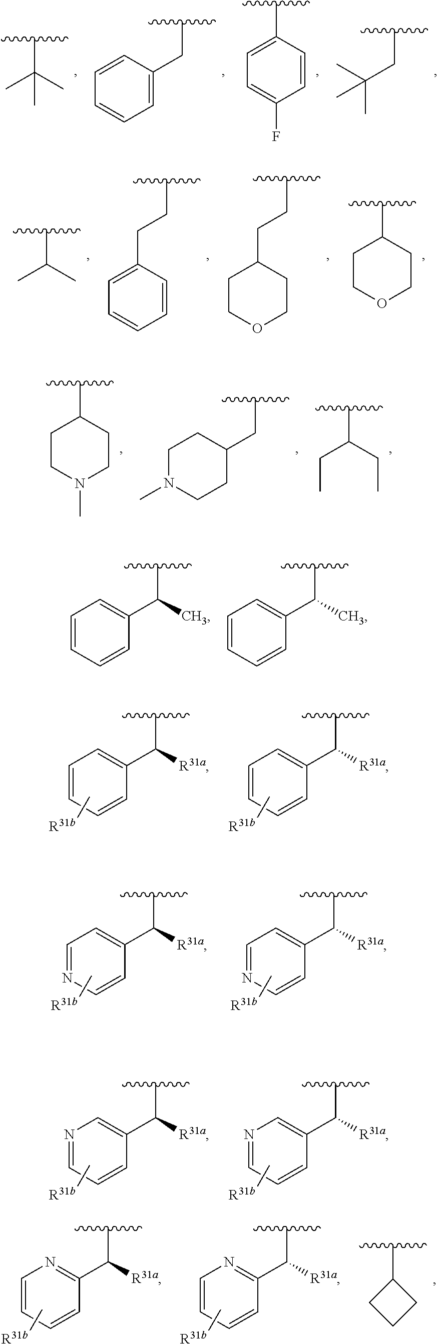

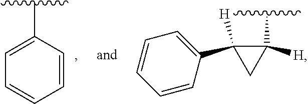

wherein R^{31a} is selected from H, D, halo, hydroxy, amino, alkylamino, C₁-C₄alkyl, and —CF₃, and

R^{31b} is selected from H, D, halo, hydroxy, amino, alkylamino, C₁-C₄alkyl, —CF₃, and —OCF₃.

In some aspects, R³¹ is:

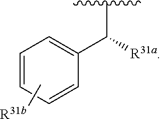

In some aspects, R^{31a} is —CH₃ and R^{31b} is halo, —CH₃, —OCF₃, or —CF₃.

In some aspects, R^{31b} is Cl.

In some aspects, R³¹ is:

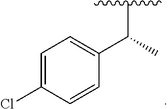 [SMILES: *[C@H](C)c1ccc(Cl)cc1]

In some aspects, the compound, or a pharmaceutically acceptable salt or solvate thereof, is selected from any one of the compounds of Table 1.

In some aspects, the compound can exhibit one or more of the following properties: (i) promote the differentiation of an OPC into an oligodendrocyte, (ii) promote the expression of a protein associated with oligodendrocyte differentiation and/or myelination (e.g., G-protein coupled receptor 17 (GPR17), myelin basic protein (MBP), ASPA, GST-pi, CC1, myelin oligodendrocyte glycoprotein (MOG), oligodendrocyte-specific protein/claudin-11, CNPase, or a combination thereof), (iii) promote the myelination of an axon, (iv) promote the remyelination of a demyelinated axon, (v) inhibit PDGFRα kinase activity, (vi) achieve a brain to plasma ratio of greater than 0.1 when systemically administered to a subject, and (vii) any combination thereof.

In some aspects, the compound can inhibit PDGFRα kinase activity.

In some aspects, the compound can inhibit PDGFRα kinase activity with an IC₅₀ of less than 500 nM, less than 400 nM, less than 300 nM, less than 200 nM, less than 100 nM, less than 75 nM, less than less than 50 nM, less than 40 nM, less than 30 nM, less than 20 nM, less than 10 nM, or less than 5 nM.

In some aspects, the IC₅₀ of the PDGFRα inhibitor is determined using an enzymatic PDGFRα kinase assay (e.g., Promega kinase assay described in Example 239).

In some aspects, the enzymatic PDGFRα kinase assay comprises 20 ng of purified PDGFRα protein, 150 uM of ATP and 1 ug of substrate, Poly (Glu4Tyr1) in a volume of 15 ul.

In some aspects, the disclosure provides a compound that can inhibit a PDGFRα activity of a cell and can further exhibit one or more of the following properties: (i) promote the differentiation of an OPC into an oligodendrocyte, (ii) promote the expression of a protein associated with oligodendrocyte differentiation and/or myelination (e.g., G-protein coupled receptor 17 (GPR17), myelin basic protein (MBP), ASPA, GST-pi, CC1, myelin oligodendrocyte glycoprotein (MOG), oligodendrocyte-specific protein/claudin-11, CNPase, or a combination thereof), (iii) promote the myelination of an axon, (iv) promote the remyelination of a demyelinated axon, (v) inhibit PDGFRα kinase activity, (vii) achieve a brain to plasma ratio of greater than 0.1 when systemically administered to a subject, or (viii) any combination thereof.

In some aspects, the compound can inhibit PDGFRα kinase activity with an IC₅₀ of less than 500 nM, less than 400 nM, less than 300 nM, less than 200 nM, less than 100 nM, less than 75 nM, less than less than 50 nM, less than 40 nM, less than 30 nM, less than 20 nM, less than 10 nM, or less than 5 nM.

In some aspects, the IC₅₀ of the PDGFRα inhibitor is determined using an enzymatic PDGFRα kinase assay (e.g., Promega kinase assay described in Example 239).

In some aspects, the enzymatic PDGFRα kinase assay comprises 20 ng of purified PDGFRα protein, 150 uM of ATP and 1 ug of substrate, Poly (Glu4Tyr1) in a volume of 15 ul.

In some aspects, the compound comprises a small molecule, an antibody, or both.

In some aspects, the small molecule comprises a compound of Formula I:

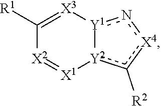

or a pharmaceutically acceptable salt or solvate thereof, wherein:
   [symbol] indicates a single bond or a double bond such that all valences are satisfied;
   X¹, X², X³, and X⁴ are selected from N and CR^{a}, with the proviso that not more than two of X¹, X², X³, and X⁴ are N;
   one of Y¹ and Y² is N and the other of Y¹ and Y² is CR^{a};
   each R^{a} is independently selected from H, halo, C₁-C₄alkyl, and C₁-C₄alkoxy;
   R¹ is selected from C₄-C₇cycloalkyl, 4-7 membered heterocyclyl, heteroaryl, aryl, and C₁-C₈alkoxy, all of which can be optionally substituted with one, two, three, four, five, or six substituents selected from halo, hydroxy, oxo, C₁-C₄alkyl, aminoC₁-C₄alkyl, hydroxyC₁-C₄alkyl, C₁-C₄alkoxy, C₁-C₄alkoxyC₁-C₄alkyl, 3-8 membered heterocyclyl, and 3-8 membered heterocyclylC₁-C₄alkyl, with the proviso that the number of substituents does not exceed the number of substitutable positions;
   R² is selected from cycloalkyl, cycloalkenyl, alkyl, oxoalkylamino, amino, heterocyclyl, heteroaryl, aminoheterocyclyl, heterocyclylamino, and aminoalkylamino, all of which can be optionally substituted with one, two, three, four, or five substituents selected from D, halo, hydroxy, oxo, and C₁-C₄alkyl;
   R² is substituted by one, two, or three R³;
   R³ is selected from aryl, heteroaryl, —C(O)R³¹, —C(O)OR³¹, —C(O)NR³¹R³², —S(O)₂NR³¹R³², —S(O)(NR³³)R³¹, —S(O)(NR³³)NR³¹R³², —C(S)NR³¹R³², C₃-C₈cycloalkyl, 3-8 membered heterocyclyl, and C₁-C₄alkyl, all of which can be optionally substituted with one, two, three, four, or five R³⁰;
   each R³⁰ is independently selected from D, halo, aryl, —OR³⁰⁰, —NR³⁰⁰R³⁰³, —S(O)ᵣR³⁰⁰, —C(O)R³⁰⁰, —C(═CR³⁴R³⁵)R³⁰⁰, and

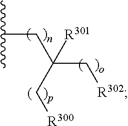

r is selected from 0, 1, and 2;
each R³⁰⁰ is independently selected from C₁-C₆alkyl, C₃-C₇cycloalkyl, aryl, heteroaryl, 3-8 membered heterocyclyl, and 3-8 membered heterocyclylaryl, all of which can be optionally substituted with one, two, three, four, or five substituents selected from D, halo, hydroxy, amino, alkylamino, cyano, C₁-C₄alkyl, C₁-C₄haloalkyl, and C₁-C₄alkoxy;
each R³⁰¹ is independently selected from H, halo, and C₁-C₄alkyl;
each R³⁰² is independently selected from H, F, hydroxy, amino, alkylamino, oxo, and C₁-C₄alkoxy;
each R³⁰³ is independently selected from H and C₁-C₄ alkyl;
n, o, and p are each independently selected from 0, 1, 2, 3, and 4;
each R³¹ is independently selected from C₁-C₈alkyl, arylC₁-C₄alkyl, heteroarylC₁-C₄alkyl, heterocyclyl, heterocyclylC₁-C₄alkyl, cycloalkyl, and cycloalkylC₁-C₄alkyl, all of which can be optionally substituted with one, two, three, four, or five substituents selected from D, halo, cyano, hydroxy, amino, —OCF₃, C₁-C₄alkyl, C₁-C₄haloalkyl, C₁-C₄alkoxy, hydroxyC₁-C₄alkyl, —S(O)₂NR³⁰⁴R³⁰⁵, —C(O)OR³⁰⁴R³⁰⁵, —C(O)NR³⁰⁴R³⁰⁵, and —NR³⁰⁴C(O)R³⁰⁵;
each R³⁰⁴ and R³⁰⁵ is independently selected from H and C₁-C₄ alkyl;
each R³² is independently selected from H and C₁-C₄alkyl; or
R³¹ and R³² together with the atom to which they are connected form a 5-8 membered heterocycyl, optionally substituted with one, two, three, four, or five substituents selected from D, halo, cyano, C₁-C₄alkyl, and C₁-C₄haloalkyl, and —C(O)NR³⁴R³⁵;
each R³⁴ and R³⁵ is independently selected from H, C₁-C₄alkyl, and C₁-C₄haloalkyl; and
each R³³ is independently selected from H, C₁-C₄alkyl, C₁-C₄haloalkyl, and —C(O)R³⁴; or
R³¹ and R³³ together with the atoms to which they are connected form a 4-8 membered heterocycyl.

In some aspects, the compound, or a pharmaceutically acceptable salt or solvate thereof, has Formula II:

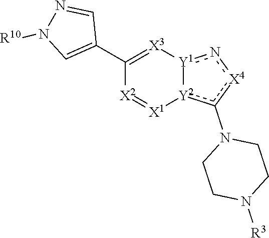

wherein R¹⁰ is selected from H, C₁-C₄alkyl, C₁-C₄alkoxy, aminoC₁-C₄alkyl, hydroxyC₁-C₄alkyl, and C₁-C₄alkylsulfonyl, wherein the C₁-C₄alkyl, C₁-C₄alkoxy, aminoC₁-C₄alkyl, hydroxyC₁-C₄alkyl, and C₁-C₄alkylsulfonyl can be optionally substituted by one or more substituents selected from hydroxyl, C₁-C₄alkoxy, NR^{10a}R^{10b}, halo, and deuterium, wherein R^{10a} and R^{10b} are selected from hydrogen and C₁-C₄alkyl, or wherein R^{10a} and R^{10b} taken together with the nitrogen atom to which they are attached form a 4- to 8-membered ring.

In some aspects, the compound, or a pharmaceutically acceptable salt or solvate thereof, has Formula III:

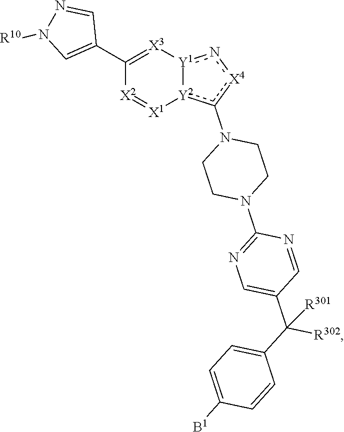

wherein B¹ is H, CI, F, or —CF₃.

In some aspects, the compound, or a pharmaceutically acceptable salt or solvate thereof, has Formula IV:

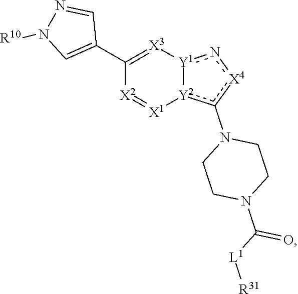

wherein L¹ is a bond or O.

In some aspects, the compound does not comprise any of the following compounds: anlotinib HCl, avapritinib, axitinib, bemcentinib, cediranib, CP-673451, dovitinib, ENMD-2076, foretinib, JNJ=10198409, JNJ=28312141, K252a, linifanib (ABT-869), masitinib, motesanib (AMG706), nintedanib, ON123300, pexidartinib (PLX3397), R81, RO4396686, seralutinib, TAK-593, tamatinib (R-406), tandutinib, telatinib, pazopanib, MK 2461, imatinib, sorafenib, or combinations thereof.

The disclosure also provides a pharmaceutical composition comprising a compound disclosed herein, or a pharmaceutically acceptable salt or solvate thereof, and a pharmaceutically acceptable excipient.

The disclosure also provides a kit comprising a compound disclosed herein, or a pharmaceutically acceptable salt or solvate thereof, or a composition disclosed herein, and instructions for use.

The disclosure also provides a method of producing a PDGFRα inhibitor comprising synthesizing a compound disclosed herein.

The disclosure also provides a compound disclosed herein, or a pharmaceutical composition disclosed herein, for use in therapy.

The disclosure also provides a method of treating a demyelinating disease in a subject in need thereof comprising administering to the subject a therapeutically effective amount of a compound disclosed herein or a pharmaceutical composition disclosed herein.

The disclosure also provides a method of improving a subject's performance in a test for assessing one or more symptoms associated with a demyelinating disease, comprising administering to the subject a therapeutically effective amount of a compound disclosed herein or a pharmaceutical composition disclosed herein, wherein after the administration, the subject's performance in the test is improved as compared to a reference subject (e.g., the subject prior to the administration).

In some aspects, the test is selected from a visual evoked potential (VEP) test, a multifocal visual evoked potential (mfVEP) test, a low contrast visual acuity (LC-VA) test, a magnetic resonance imaging (MRI) (e.g., magnetic transfer resonance, myelin water fraction (MWF), and quantitative susceptibility mapping (QSM)), an electromyography (EMG), a nerve conduction velocity (NCV) test, an Extended Disability Status Scale (EDSS), a timed walk test (e.g., timed 25-foot walk), a Nine-Hole Peg Test (9HPT), an ocular coherence tomograph (OCT), a quality of life measure test (e.g., Multiple Sclerosis Quality of Life-54 and Vision-Related Quality of Life), cognitive assessment (e.g., Montreal Cognitive Assessment), or combinations thereof.

In some aspects, the demyelinating disease comprises an acute disseminated encephalomyelitis (ADEM), acute hemorrhagic leukoencephalitis, acute transverse myelitis, adrenoleukodystrophy, adrenomyeloneuropathy, Alexander disease, Alzheimer's disease, aminoacidurias, amyotrophic lateral sclerosis, anti-MAG peripheral neuropathy, anti-MOG associated spectrum, Balo concentric sclerosis, brain injury, CAMFAK syndrome, Canavan disease, carbon monoxide toxicity, central pontine myelinolysis, cerebral hypoxia, cerebral ischemia, Charcot-Marie-Tooth disease, chronic inflammatory demyelinating polyneuropathy, chronic traumatic encephalopathy, clinically isolated syndrome (CIS), congenital cataract, copper deficiency associated condition, delayed post-hypoxic leukoencephalopathy, diffuse cerebral sclerosis of Schilder, diffuse myelinoclastic sclerosis, extrapontine myelinolysis Gaucher disease, Guillain-Barré syndrome, hereditary neuropathy, hereditary neuropathy with liability to pressure palsy, HTLV-1-associated myelopathy, Hurler syndrome, hypomyelination, hypoxic brain injury, Krabbe disease, Leber hereditary optic atrophy and related mitochondrial disorders, leukodystrophic disorders, Marchiafava-Bignami disease, metachromatic leukodystrophy, multiple sclerosis (e.g., primary progressive multiple sclerosis (PPMS), relapsing-remitting multiple sclerosis (RRMS), secondary progressive multiple sclerosis (SPMS), progressive relapsing multiple sclerosis, Marburg multiple sclerosis, tumefactive multiple sclerosis, and optic-spinal multiple sclerosis), multiple system atrophy, myelinoclastic disorders, myelopathy, nerve injury, neuromyelitis optica (NMO), Niemann-Pick disease, optic neuropathy, optic neuritis (e.g., acute optic neuritis and chronic relapsing inflammatory optic neuritis (CRION)), osmotic demyelination syndrome, Parkinson's disease, Pelizaeus-Merzbacher disease, peripheral neuropathy, phenylketonuria, progressive inflammatory neuropathy, progressive multifocal leukoencephalopathy, progressive subcortical ischemic demyelination, reperfusion injury, Schilder disease, solitary sclerosis, spinal cord injury, subacute sclerosing panencephalitis, Tabes dorsalis, Tay-Sachs disease, transverse myelitis, traumatic brain injury, tropical spastic paraparesis, vitamin B12 deficiency, cerebral palsy, or a combination thereof.

In some aspects, the demyelinating disease is characterized by demyelination of one or more cells within the CNS of the subject.

In some aspects, the demyelinating disease is multiple sclerosis.

In some aspects, the multiple sclerosis comprises a clinically isolated syndrome (“CIS”), relapsing-remitting MS (“RRMS”), secondary progressive MS (“SPMS”), primary progressive MS (“PPMS”), optic neuritis or transverse myelitis.

In some aspects, the demyelinating disease is an optic neuritis.

In some aspects, treating the demyelinating disease comprises reducing one or more symptoms associated with the demyelinating disease.

In some aspects, the one or more symptoms comprise fatigue, dizziness, malaise, elevated fever and high body temperature, extreme sensitivity to cold in the hands and feet, weakness and stiffness in muscles and joints, weight changes, digestive or gastrointestinal problems, low blood pressure, high blood pressure, irritability, anxiety, depression, impaired vision (e.g., blurred vision, double vision, reduction in low contrast visual acuity (LC-VA)), ataxia, clonus, spasms, dysarthria, weakness, clumsiness, hand paralysis, hemiparesis, genital anesthesia, sexual dysfunction, incoordination, paresthesias, ocular paralysis, impaired muscle coordination, loss of sensation, tingling, numbness, pain, neurological symptoms, impaired cognition, unsteady gait, balance problems, dizziness, spastic paraparesis, incontinence, hearing problems, speech problems, loss of olfaction, agusia, or combinations thereof.

The disclosure also provides a method of promoting the myelination of an axon in a subject in need thereof, the method comprising administering to the subject an effective amount of a compound disclosed herein or a pharmaceutical composition disclosed herein.

In some aspects, promoting the myelination of an axon results in an increase in the expression of one or more of the following markers within the subject: myelin basic protein (MBP) Myelin Oligodendrocyte Glycoprotein (MOG), Oligodendrocyte Specific Protein/Claudin-11, CNPase, or any combination thereof.

In some aspects, the myelination of an axon can be determined by visualizing and/or quantifying the expression of one or more of the following markers: myelin basic protein (MBP) Myelin Oligodendrocyte Glycoprotein (MOG), Oligodendrocyte Specific Protein/Claudin-11, CNPase, or any combination thereof.

The disclosure also provides method of promoting the remyelination of a demyelinated axon in a subject in need thereof, the method comprising administering to the subject an effective amount of a compound disclosed herein or a pharmaceutical composition disclosed herein.

In some aspects, promoting the remyelination of a demyelinated axon results in an increase in the expression of one or more of the following markers within the subject: myelin basic protein (MBP) Myelin Oligodendrocyte Glycoprotein (MOG), Oligodendrocyte Specific Protein/Claudin-11, CNPase, or any combination thereof.

In some aspects, the remyelination of a demyelinated axon can be determined by visualizing and/or quantifying the expression of one or more of the following markers: myelin basic protein (MBP) Myelin Oligodendrocyte Glycoprotein (MOG), Oligodendrocyte Specific Protein/Claudin-11, CNPase, or any combination thereof.

The disclosure also provides a method of reducing the demyelination of a myelinated neuronal axon in a subject in need thereof, the method comprising administering to the subject an effective amount of a compound disclosed herein or a pharmaceutical composition disclosed herein.

In some aspects, reducing the demyelination of a myelinated neuronal axon results in an increase in the expression of one or more of the following markers: myelin basic protein (MBP) Myelin Oligodendrocyte Glycoprotein (MOG), Oligodendrocyte Specific Protein/Claudin-11, CNPase, or any combination thereof.

In some aspects, the reduction in the demyelination of a myelinated neuronal axon can be determined by visualizing and/or quantifying the expression of one or more of the following markers: myelin basic protein (MBP) Myelin Oligodendrocyte Glycoprotein (MOG), Oligodendrocyte Specific Protein/Claudin-11, CNPase, or any combination thereof.

The present disclosure also provides a method of activating an oligodendrocyte progenitor cell (OPC) within the central nervous system (CNS) of a subject in need thereof, the method comprising administering to the subject an effective amount of a compound disclosed herein or a pharmaceutical composition disclosed herein.

In some aspects, the subject has, or is at risk of developing a demyelinating disease, for example a disease disclosed herein.

In some aspects, the method is a method of treating or preventing a demyelinating disease, for example a disease disclosed herein.

In some aspects, the compound or the pharmaceutical composition is administered to the subject once.

In some aspects, the compound or the pharmaceutical composition is administered to the subject more than once using intermittent dosing.

In some aspects, the intermittent dosing comprises administering the PDGFRα inhibitor to the subject every other day, every three days, every four days, every five days, every six days, once a week, every eight days, every nine days, every 10 days, every 11 days, every 12 days, every 13 days, once every two weeks, once every three weeks, once a month, once every two months, once every three months, once every four months, once every five months, once every six months, or once every twelve months.

In some aspects, the intermittent dosing comprises administering to the subject a first dose and a second dose of the PDGFRα inhibitor, wherein the second dose is administered at least one day, at least two days, at least three days, at least four days, at least five days, at least six days, at least seven days, at least eight days, at least nine days, at least 10 days, at least 11 days, at least 12 days, at least 13 days, at least two weeks, at least three weeks, at least one month, at least two months, at least three months, at least four months, at least five months, at least six months, or at least 12 months after administering the first dose.

In some aspects, the second dose is administered to the subject one day, two days, three days, four days, five days, six days, seven days, eight days, nine days, 10 days, 11 days, 12 days, 13 days, two weeks, three weeks, one month, two months, three months, four months, five months, six months, or 12 months after administering the first dose.

In some aspects, after the administration, the compound or the pharmaceutical composition can achieve a brain to plasma ratio of greater than 0.1, greater than 0.2, greater than 0.3, greater than 0.4, greater than 0.5, greater than 0.6, greater than 0.7, greater than 0.8, greater than 0.9, greater than 1.0, greater than 1.1, greater than 1.2, greater than 1.3, greater than 1.4, greater than 1.5, greater than 1.6, greater than 1.7, greater than 1.8, greater than 1.9, or greater than 2.0.

In some aspects, the method further comprises administering to the subject an additional therapeutic agent.

In some aspects, the additional therapeutic agent comprises a standard care of treatment.

In some aspects, the additional therapeutic agent comprises an immunomodulatory agent.

In some aspects, the additional therapeutic agent is selected from interferon beta-1b, interferon beta-1a, peginterferon beta-1a, alemtuzumab, natalizumab, ocrelizumab, ofatumumab, glatiramer acetate, teriflunomide, dimethyl fumarate, monomethyl fumarate, diroximel fumarate, fingolimod hydrochloride, siponimod fumaric acid, ozanimod hydrochloride, BTK inhibitor, or a pharmaceutically acceptable salt thereof

In some aspects, the additional therapeutic agent is administered to the subject prior to, concurrently, or after the administration of the compound or the pharmaceutical composition.

The disclosure also provides a method of inducing the differentiation of an oligodendrocyte progenitor cell (OPC) into an oligodendrocyte, the method comprising contacting the OPC with an effective amount of a compound disclosed herein or a pharmaceutical composition disclosed herein.

In some aspects, inducing the differentiation of the OPC into an oligodendrocyte results in an increase in the expression of the following markers in the subject: GPR17, MBP, ASPA, GST-pi, CC1, myelin oligodendrocyte glycoprotein (MOG), oligodendrocyte-specific protein/claudin-11, CNPase, or a combination thereof.

In some aspects, the differentiation of the OPC into an oligodendrocyte is measured by determining the expression of GPR17, MBP, ASPA, GST-pi, CC1, myelin oligodendrocyte glycoprotein (MOG), oligodendrocyte-specific protein/claudin-11, CNPase, or a combination thereof.

The disclosure also provides a method of inhibiting PDGFRα activity in a cell, the method comprising contacting the cell with an effective amount of a compound disclosed herein or a pharmaceutical composition disclosed herein.

In some aspects, the inhibition of the PDGFRα activity is measured by one or more of the following: an in vitro OPC differentiation assay (e.g., as described in Example 240), a cuprizone model for demyelination (e.g., as described in Example 243), an in vivo OPC differentiation assay (e.g., as described in Example 245), an enzymatic PDGFRα kinase assay (e.g., as described in Example 239), or any combination thereof.

In some aspects, the contacting occurs ex vivo or in vivo.

In some aspects, the method is a method of treating by a therapy.

The disclosure also provides a method of treating a demyelinating disease in a subject in need thereof, the method comprising administering to the subject an effective amount of a PDGFRα inhibitor, optionally wherein the PDGFRα inhibitor can induce the differentiation of an oligodendrocyte progenitor cell (OPC) into an oligodendrocyte.

In some aspects, inducing the differentiation of the OPC into an oligodendrocyte results in an increase in the expression of the following markers in the subject: GPR17, MBP, ASPA, GST-pi, CC1, myelin oligodendrocyte glycoprotein (MOG), oligodendrocyte-specific protein/claudin-11, CNPase, or a combination thereof.

In some aspects, inducing the differentiation of the OPC into an oligodendrocyte is measured by determining the expression of GPR17, MBP, ASPA, GST-pi, CC1, myelin oligodendrocyte glycoprotein (MOG), oligodendrocyte-specific protein/claudin-11, CNPase, or a combination thereof.

The disclosure also provides a method of improving a subject's performance in a test for assessing one or more symptoms associated with a demyelinating disease, comprising administering to the subject a therapeutically effective amount of a PDGFRα inhibitor, optionally wherein the PDGFRα inhibitor is capable of inducing the differentiation of an oligodendrocyte progenitor cell (OPC) into an oligodendrocyte.

In some aspects, the test is selected from a visual evoked potential (VEP) test, a multifocal visual evoked potential (mfVEP) test, a low contrast visual acuity (LC-VA) test, a magnetic resonance imaging (MRI) (e.g., magnetic transfer resonance, myelin water fraction (MWF), and quantitative susceptibility mapping (QSM)), an electromyography (EMG), a nerve conduction velocity (NCV) test, an Extended Disability Status Scale (EDSS), a timed walk test (e.g., timed 25-foot walk), a Nine-Hole Peg Test (9HPT), an ocular coherence tomograph (OCT), a quality of life measure test (e.g., Multiple Sclerosis Quality of Life-54 and Vision-Related Quality of Life), cognitive assessment (e.g., Montreal Cognitive Assessment), or combinations thereof.

In some aspects, the demyelinating disease comprises an acute disseminated encephalomyelitis (ADEM), acute hemorrhagic leukoencephalitis, acute transverse myelitis, adrenoleukodystrophy, adrenomyeloneuropathy, Alexander disease, Alzheimer's disease, aminoacidurias, amyotrophic lateral sclerosis, anti-MAG peripheral neuropathy, anti-MOG associated spectrum, Balo concentric sclerosis, brain injury, CAMFAK Syndrome, Canavan disease, carbon monoxide toxicity, central pontine myelinolysis, cerebral hypoxia, cerebral ischemia, Charcot-Marie-Tooth chronic disease, inflammatory demyelinating polyneuropathy, chronic traumatic encephalopathy, clinically isolated syndrome (CIS), congenital cataract, copper deficiency associated condition, delayed post-hypoxic leukoencephalopathy, diffuse cerebral sclerosis of Schilder, diffuse myelinoclastic sclerosis, extrapontine myelinolysis Gaucher disease, Guillain-Barré syndrome, hereditary neuropathy, hereditary neuropathy with liability to pressure palsy, HTLV-1-associated myelopathy, Hurler syndrome, hypomyelination, hypoxic brain injury, Krabbe disease, Leber hereditary optic atrophy and related mitochondrial disorders, leukodystrophic disorders, Marchiafava-Bignami disease, metachromatic leukodystrophy, multiple sclerosis (e.g., primary progressive multiple sclerosis (PPMS), relapsing-remitting multiple sclerosis (RRMS), secondary progressive multiple sclerosis (SPMS), progressive relapsing multiple sclerosis, Marburg multiple sclerosis, tumefactive multiple sclerosis, and optic-spinal multiple sclerosis), multiple system atrophy, myelinoclastic disorders, myelopathy, nerve injury, neuromyelitis optica (NMO), Niemann-Pick disease, optic neuropathy, optic neuritis (e.g., acute optic neuritis and chronic relapsing inflammatory optic neuritis (CRION)), osmotic demyelination syndrome, Parkinson's disease, Pelizaeus-Merzbacher disease, peripheral neuropathy, phenylketonuria, progressive inflammatory neuropathy, progressive multifocal leukoencephalopathy, progressive subcortical ischemic demyelination, reperfusion injury, Schilder disease, solitary sclerosis, spinal cord injury, subacute sclerosing panencephalitis, Tabes dorsalis, Tay-Sachs disease, transverse myelitis, traumatic brain injury, tropical spastic paraparesis, vitamin B12 deficiency, cerebral palsy, or a combination thereof.

In some aspects, the demyelinating disease is characterized by demyelination of one or more cells within the CNS of the subject.

In some aspects, the demyelinating disease is a multiple sclerosis.

In some aspects, the multiple sclerosis comprises a clinically isolated syndrome (“CIS”), relapsing-remitting MS (“RRMS”), secondary progressive MS (“SPMS”), primary progressive MS (“PPMS”), or transverse myelitis.

In some aspects, the demyelinating disease is an optic neuritis.

In some aspects, treating the demyelinating disease comprises reducing one or more symptoms associated with the demyelinating disease.

In some aspects, the one or more symptoms comprise fatigue, dizziness, malaise, elevated fever and high body temperature, extreme sensitivity to cold in the hands and feet, weakness and stiffness in muscles and joints, weight changes, digestive or gastrointestinal problems, low blood pressure, high blood pressure, irritability, anxiety, depression, impaired vision (e.g., blurred vision, double vision, reduction in low contrast visual acuity (LC-VA)), ataxia, clonus, spasms, dysarthria, weakness, clumsiness, hand paralysis, hemiparesis, genital anesthesia, sexual dysfunction, incoordination, paresthesias, ocular paralysis, impaired muscle coordination, loss of sensation, tingling, numbness, pain, neurological symptoms, impaired cognition, unsteady gait, balance problems, dizziness, spastic paraparesis, incontinence, hearing problems, speech problems, loss of olfaction, agusia, or combinations thereof

The disclosure also provides a method of promoting the myelination of an axon in a subject in need thereof, the method comprising administering to the subject an effective amount of a PDGFRα inhibitor, optionally wherein the PDGFRα inhibitor is capable of inducing the differentiation of an oligodendrocyte progenitor cell (OPC) into an oligodendrocyte.

In some aspects, promoting the myelination of an axon results in an increase in the expression of the following markers in the subject: myelin basic protein (MBP) Myelin Oligodendrocyte Glycoprotein (MOG), Oligodendrocyte Specific Protein/Claudin-11, CNPase, or any combination thereof.

In some aspects, the myelination of an axon can be determined by visualizing and/or quantifying the expression of one or more of the following markers: myelin basic protein (MBP) Myelin Oligodendrocyte Glycoprotein (MOG), Oligodendrocyte Specific Protein/Claudin-11, CNPase, or any combination thereof.

The disclosure also provides a method of promoting the remyelination of a demyelinated axon in a subject in need thereof, the method comprising administering to the subject an effective amount of a PDGFRα inhibitor, optionally wherein the PDGFRα inhibitor is capable of inducing the differentiation of an oligodendrocyte progenitor cell (OPC) into an oligodendrocyte.

In some aspects, promoting the remyelination of a demyelinated axon results in an increase in the expression of the following markers in the subject: myelin basic protein (MBP) Myelin Oligodendrocyte Glycoprotein (MOG), Oligodendrocyte Specific Protein/Claudin-11, CNPase, or any combination thereof.

In some aspects, the remyelination of a demyelinated axon can be determined by visualizing and/or quantifying the expression of one or more of the following markers: myelin basic protein (MBP) Myelin Oligodendrocyte Glycoprotein (MOG), Oligodendrocyte Specific Protein/Claudin-11, CNPase, or any combination thereof.

The disclosure also provides a method of reducing the demyelination of a myelinated axon in a subject in need thereof, the method comprising administering to the subject an effective amount of a PDGFRα inhibitor, optionally wherein the PDGFRα inhibitor is capable of inducing the differentiation of an oligodendrocyte progenitor cell (OPC) into an oligodendrocyte.

In some aspects, reducing the reduction in the demyelination of a myelinated axon results in an increase in the expression of the following markers in the subject: myelin basic protein (MBP) Myelin Oligodendrocyte Glycoprotein (MOG), Oligodendrocyte Specific Protein/Claudin-11, CNPase, or any combination thereof.

In some aspects, the reduction in the demyelination of a myelinated neuronal axon can be determined by visualizing and/or quantifying the expression of one or more of the following markers: myelin basic protein (MBP) Myelin Oligodendrocyte Glycoprotein (MOG), Oligodendrocyte Specific Protein/Claudin-11, CNPase, or any combination thereof.

The disclosure also provides method of activating an oligodendrocyte progenitor cell (OPC) within the central nervous system (CNS) of a subject in need thereof, the method comprising administering to the subject an effective amount of a PDGFRα inhibitor, optionally wherein the PDGFRα inhibitor is capable of inducing the differentiation of an oligodendrocyte progenitor cell (OPC) into an oligodendrocyte.

In some aspects, the method is a method of treatment by a therapy.

In some aspects, the PDGFRα inhibitor is administered to the subject once.

In some aspects, the PDGFRα inhibitor is administered to the subject using intermittent dosing.

In some aspects, the intermittent dosing comprises administering the PDGFRα inhibitor to the subject every other day, every three days, every four days, every five days, every six days, once a week, every eight days, every nine days, every 10 days, every 11 days, every 12 days, every 13 days, once every two weeks, once every three weeks, once a month, once every two months, once every three months, once every four months, once every five months, once every six months, or once every twelve months.

In some aspects, the intermittent dosing comprises administering to the subject a first dose and a second dose of the PDGFRα inhibitor, wherein the second dose is administered at least one day, at least two days, at least three days, at least four days, at least five days, at least six days, at least seven days, at least eight days, at least nine days, at least 10 days, at least 11 days, at least 12 days, at least 13 days, at least two weeks, at least three weeks, at least one month, at least two months, at least three months, at least four months, at least five months, at least six months, or at least 12 months after administering the first dose.

In some aspects, the second dose is administered to the subject one day, two days, three days, four days, five days, six days, seven days, eight days, nine days, 10 days, 11 days, 12 days, 13 days, two weeks, three weeks, one month, two months, three months, four months, five months, six months, or 12 months after administering the first dose.

In some aspects, after the administration, the PDGFRα inhibitor can achieve a brain to plasma ratio of greater than 0.1, greater than 0.2, greater than 0.3, greater than 0.4, greater than 0.5, greater than 0.6, greater than 0.7, greater than 0.8, greater than 0.9, greater than 1.0, greater than 1.1, greater than 1.2, greater than 1.3, greater than 1.4, greater than 1.5, greater than 1.6, greater than 1.7, greater than 1.8, greater than 1.9, or greater than 2.0.

In some aspects, the method further comprises administering to the subject an additional therapeutic agent.

In some aspects, the additional therapeutic agent comprises a standard care of treatment.

In some aspects, the additional therapeutic agent comprises an immunomodulatory agent.

In some aspects, the additional therapeutic agent is selected from interferon beta-1b, interferon beta-1a, peginterferon beta-1a, alemtuzumab, natalizumab, ocrelizumab, ofatumumab, glatiramer acetate, teriflunomide, dimethyl fumarate, monomethyl fumarate, diroximel fumarate, fingolimod hydrochloride, siponimod fumaric acid, ozanimod hydrochloride, BTK inhibitor, or a pharmaceutically acceptable salt thereof.

In some aspects, the additional therapeutic agent is administered to the subject prior to, concurrently, or after the administration of the compound or the pharmaceutical composition.

The disclosure also provides a method of inducing the differentiation of an oligodendrocyte progenitor cell (OPC) into an oligodendrocyte, the method comprising contacting the OPC with an effective amount of a PDGFRα inhibitor.

In some aspects, the method is a method of treatment by therapy.

In some aspects, inducing the differentiation of the OPC into an oligodendrocyte results in an increase in the expression of the following marker in the subject: GPR17, MBP, ASPA, GST-pi, CC1, myelin oligodendrocyte glycoprotein (MOG), oligodendrocyte-specific protein/claudin-11, CNPase, or a combination thereof.

In some aspects, the differentiation of the OPC into an oligodendrocyte is measured by determining the expression of GPR17, MBP, ASPA, GST-pi, CC1, myelin oligodendrocyte glycoprotein (MOG), oligodendrocyte-specific protein/claudin-11, CNPase, or a combination thereof.

In some aspects, the contacting occurs ex vivo or in vivo.

In some aspects, the PDGFRα inhibitor comprises a small molecule, an antibody, or both.

In some aspects, the PDGFRα inhibitor is a small molecule that can inhibit the activity of a kinase of PDGFRα.

In some aspects, the PDGFRα inhibitor is an antibody that can bind to the extracellular region of PDGFRα.

In some aspects, the PDGFRα inhibitor can inhibit the activity of PDGFRα in the subject with an IC₅₀ of less than 500 nM, less than 400 nM, less than 300 nM, less than 200 nM, less than 100 nM, less than 75 nM, less than less than 50 nM, less than 40 nM, less than 30 nM, less than 20 nM, less than 10 nM, or less than 5 nM.

In some aspects, the IC₅₀ of the PDGFRα inhibitor is determined using an enzymatic PDGFRα kinase assay (e.g., Promega kinase assay described in Example 239).

In some aspects, the enzymatic PDGFRα kinase assay comprises 20 ng of purified PDGFRα protein, 150 uM of ATP and 1 μg of substrate, Poly (Glu4Tyr1) in a volume of 15 ul.

The disclosure also provides a method of treating a relapsing form of multiple sclerosis in a subject in need thereof, the method comprising administering to the subject an effective amount of a compound disclosed herein or a pharmaceutical composition disclosed herein.

The disclosure also provides a method of treating a relapsing form of multiple sclerosis in a subject in need thereof, the method comprising administering to the subject an effective amount of a PDGFRα inhibitor, optionally, wherein the PDGFRα inhibitor is capable of inducing the differentiation of an oligodendrocyte progenitor cells (OPC) into an oligodendrocyte.

In some aspects, inducing the differentiation of the OPC into an oligodendrocyte results in an increase in the expression of the following marker in the subject: GPR17, MBP, ASPA, GST-pi, CC1, myelin oligodendrocyte glycoprotein (MOG), oligodendrocyte-specific protein/claudin-11, CNPase, or a combination thereof.

In some aspects, the differentiation of the OPC into an oligodendrocyte is measured by determining the expression of GPR17, MBP, ASPA, GST-pi, CC1, myelin oligodendrocyte glycoprotein (MOG), oligodendrocyte-specific protein/claudin-11, CNPase, or a combination thereof.

In some aspects, the relapsing form of multiple sclerosis comprises a clinically isolated syndrome (“CIS”), relapsing-remitting MS (“RRMS”), secondary progressive MS (“SPMS”), primary progressive MS (“PPMS”), or transverse myelitis.

The disclosure also provides a compound disclosed herein or a pharmaceutical composition disclosed herein, for use in a method of one or more of the following: (i) promote the differentiation of an OPC into an oligodendrocyte, (ii) promote the expression of a protein associated with oligodendrocyte differentiation and/or myelination (e.g., G-protein coupled receptor 17, myelin basic protein (MBP), ASPA, GST-pi, CC1, myelin oligodendrocyte glycoprotein (MOG), oligodendrocyte-specific protein/claudin-11, CNPase, or a combination thereof), (iii) promote the myelination of an axon, (iv) promote the remyelination of a demyelinated axon, (v) inhibit PDGFRα kinase activity, (vi) achieve a brain to plasma ratio of greater than 0.1 when systemically administered to a subject, and (vii) any combination thereof.

The disclosure also provides a compound that can inhibit a PDGFRα activity of a cell (“PDGFRα inhibitor”), for use in a method of one or more of the following: (i) promote the differentiation of an OPC into an oligodendrocyte, (ii) promote the expression of a protein associated with oligodendrocyte differentiation and/or myelination (e.g., G-protein coupled receptor 17 (GPR17), myelin basic protein (MBP), ASPA, GST-pi, CC1, myelin oligodendrocyte glycoprotein (MOG), oligodendrocyte-specific protein/claudin-11, CNPase, or a combination thereof), (iii) promote the myelination of an axon, (iv) promote the remyelination of a demyelinated axon, (v) inhibit PDGFRα kinase activity, (vi) achieve a brain to plasma ratio of greater than 0.1 when systemically administered to a subject, or (vii) any combination thereof.

The disclosure also provides a method of treating a demyelinating disease in a subject in need thereof, the method comprising administering to the subject an effective amount of a PDGFRα inhibitor, optionally, wherein the PDGFRα inhibitor can treat the demyelinating disease by inducing the differentiation of an oligodendrocyte progenitor cell (OPC) into an oligodendrocyte.

The disclosure also provides a method of promoting the myelination of a neuronal axon in a subject in need thereof, the method comprising administering to the subject an effective amount of a PDGFRα inhibitor, optionally, wherein the PDGFRα inhibitor can promote the myelination of a neuronal axon by inducing the differentiation of an oligodendrocyte progenitor cell (OPC) into an oligodendrocyte.

The disclosure also provides a method of promoting the remyelination of a demyelinated neuronal axon in a subject in need thereof, the method comprising administering to the subject an effective amount of a PDGFRα inhibitor, optionally, wherein the PDGFRα inhibitor can promote the remyelination of the demyelinated neuronal axon by inducing the differentiation of an oligodendrocyte progenitor cell (OPC) into an oligodendrocyte.

The disclosure also provides a method of reducing the demyelination of a myelinated neuronal axon in a subject in need thereof, the method comprising administering to the subject an effective amount of a PDGFRα inhibitor, optionally, wherein the PDGFRα inhibitor can reduce the demyelination of the myelinated neuronal axon by inducing the differentiation of an oligodendrocyte progenitor cell (OPC) into an oligodendrocyte.

The disclosure also provides a method of activating an oligodendrocyte progenitor cell (OPC) within the central nervous system (CNS) of a subject in need thereof, the method comprising administering to the subject an effective amount of a PDGFRα inhibitor, optionally, wherein the PDGFRα inhibitor can activate the OPC within the CNS by inducing the differentiation of the OPC into an oligodendrocyte.

The disclosure also provides a method of treating a PDGF-associated tumor in a subject in need thereof, comprising administering to the subject a therapeutically effective amount of a compound disclosed herein or a pharmaceutical composition disclosed herein, wherein after the administration, PDGFRα activity is reduced in the subject.

The disclosure also provides a method of treating a PDGF-associated tumor in a subject in need thereof, comprising administering to the subject a therapeutically effective amount of PDGFRα inhibitor.

In some aspects, the PDGF-associated tumor comprises an oligodendroglioma.

In some aspects, the method is a method of treatment by therapy.

### BRIEF DESCRIPTION OF THE DRAWINGS

FIGS. 1A and 1B provide comparison of the percentage of OPCs that have differentiated into MBP+ oligodendrocytes after in vitro treatment with activity blocking anti-PDGFRα or anti-PDGFRβ antibodies, respectively. OPCs treated with an IgG isotype antibody were used as control. All antibodies were used at four different concentrations: 10 μg/mL, 1 μg/mL, 0.1 μg/mL, and 0.01 μg/mL. Bar graphs display mean±SEM. **** indicates a P-value of <0.0001, one-way ANOVA.

FIGS. 2A and 2B provide representative immunohistochemistry images comparing the in vivo generation of new oligodendrocytes in the brains of mice treated with one of the following: (i) vehicle control (FIG. 2A), or (ii) PDGFRα inhibitor (Compound 6) (PO 5 mg/kg; single dose) (FIG. 2B). The newly generated oligodendrocytes are identified based on upregulation of GPR 17 expression.

FIG. 3 provides quantitative data demonstrating the dose responsive induction of newly generated oligodendrocytes in vivo. Healthy mice received a single oral dose of 0.1 (“2”), 0.33 (“3”), 1 (“4”), or 3.3 (“5”) mg/kg of PDGFRα inhibitor (Compound 26). Control mice received a vehicle control (“1”). The number of newly generated, GPR17+, oligodendrocytes/mm² was quantitated via automated image analysis from sections spanning the anterio-posterior axis of the brain. Bar graphs display mean±SD. * and **** indicate P-values of 0.05 and <0.0001 respectively, relative to vehicle, one-way ANOVA, n=8 mice per group.

FIGS. 4A and 4B provide a comparison of in vivo remyelination in the brains of demyelinated mice treated with one of the following: (i) vehicle control (FIG. 4A), or (ii) PDGFRα inhibitor (Compound 6) (5 mg/kg; single oral dose) (FIG. 4B). Demyelination was induced by cuprizone intoxication over 17 months. Myelin was visualized via myelin basic protein staining.

FIG. 5 provides a quantitative comparison of the data provided in FIGS. 4A and 4B. As further control, MBP expression in the following animals are also shown: (i) normal healthy mice (“1”); (ii) mice treated with Thyroid Hormone (T3) (10 mg/kg) (“3”); (iii) mice treated with anti-Lingo antibody (5 mg/kg) (“4”); and (iv) mice treated with clemastine (75 mg/kg) (“5”). Bar graphs display mean±SD. * and **** indicate a P-values of 0.05 and <0.0001 respectively, relative to vehicle, one-way ANOVA, n=8 mice per group.

FIG. 6 shows the number of newly generated oligodendrocytes (based on GPR17 expression/mm²) in the brain tissue of mice after intermittent dosing of a PDGFRα inhibitor (Compound 78). The different treatment groups shown include: (1) single dose of the vehicle control on day 1; (2) a dose of the vehicle control on day 1 and a dose of Compound 78 compound on day 5; (3) a first dose of Compound 78 on day 1 and a second dose of Compound 78 on day 5; (4) a first dose of Compound 78 on day 1 and a second dose of Compound 78 on day 14; (5) a first dose of Compound 78 on day 1 and a second dose of Compound 78 on day 21.

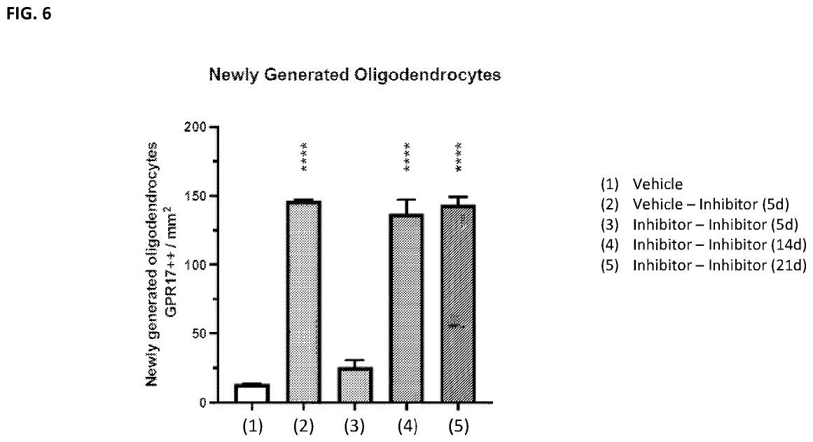

### DETAILED DESCRIPTION OF THE DISCLOSURE

The present disclosure describes the surprising discovery that inhibition of PDGFRα activity can induce oligodendrocyte progenitor cell (OPC) differentiation and myelination, including remyelination. Further, present disclosure identifies compounds (e.g., small molecules and antibodies) that can induce the differentiation of OPCs into cells that have features of mature oligodendrocytes, including morphological characteristics and the protein expression patterns associated with myelination, and can also affect remyelination. Accordingly, as described herein, the compounds of the present disclosure can be useful in treating various diseases, such as those associated with demyelination. Additional aspects of the present disclosure are provided throughout the present application.

Before the present disclosure is described in greater detail, it is to be understood that this disclosure is not limited to the particular compositions or process steps described, which can, of course, vary. As will be apparent to those of skill in the art upon reading this disclosure, each of the individual aspects described and illustrated herein has discrete components and features which can be readily separated from, or combined with, the features of any of the other several aspects without departing from the scope or spirit of the present disclosure. Any recited method can be carried out in the order of events recited or in any other order which is logically possible.

The headings provided herein are not limitations of the various aspects of the disclosure, which can be defined by reference to the specification as a whole. It is also to be understood that the terminology used herein is for the purpose of describing particular aspects only, and is not intended to be limiting, since the scope of the present disclosure will be limited only by the appended claims.

### I. Terms

In order that the present disclosure can be more readily understood, certain terms are first defined. As used in this application, except as otherwise expressly provided herein, each of the following terms shall have the meaning set forth below. Additional definitions are set forth throughout the application.

The term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 5th ed., 2013, Academic Press; and the Oxford Dictionary of Biochemistry and Molecular Biology, 2nd ed., 2008, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited (e.g., 10), it is to be understood that values that are about the same quantity or amount as the recited value (e.g., ±10%) are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

The term “halo” as used herein by itself or as part of another group refers to Cl, F, Br, or I.

The term “nitro” as used herein by itself or as part of another group refers to NO₂.

The term “cyano” as used herein by itself or as part of another group refers to CN.

The term “hydroxy” as herein used by itself or as part of another group refers to OH.

The term “alkyl” as used herein by itself or as part of another group refers to a straight- or branched-chain aliphatic hydrocarbon containing one to twelve carbon atoms, i.e., a C₁-C₁₂alkyl, or the number of carbon atoms designated, e.g., a C₁alkyl such as methyl, a C₂alkyl such as ethyl, etc. In some aspects, the alkyl is a C₁-C₁₀alkyl. In some aspects, the alkyl is a C₁-C₆alkyl. In some aspects, the alkyl is a C₁-C₄alkyl. In some aspects, the alkyl is a C₁-C₃alkyl, i.e., methyl, ethyl, propyl, or isopropyl. Non limiting exemplary C₁-C₁₂ alkyl groups include methyl, ethyl, propyl, isopropyl, butyl, sec-butyl, tert-butyl, iso-butyl, 3-pentyl, hexyl, heptyl, octyl, nonyl, and decyl. In some aspects, one or more of the hydrogen atoms of the alkyl group are replaced by deuterium atoms, i.e., the alkyl group is isotopically-labeled with deuterium. A non-limiting exemplary deuterated alkyl group is —CD₃.

The term “haloalkyl” as used herein by itself or as part of another group refers to an alkyl group wherein one or more hydrogen atoms of the alkyl group are replaced by halo atoms. In some aspects, the haloalkyl group is a —CF₃ group.

The term “alkoxy” as used herein by itself or as part of another group refers to an alkyl group attached to a terminal oxygen atom. In some aspects, the alkyl is a C₁-C₈alkyl and resulting alkoxy is thus referred to as a “C₁-C₈alkoxy.” In some aspects, the alkyl is a C₁-C₄alkyl group. Non-limiting exemplary alkoxy groups include methoxy, ethoxy, and tert-butoxy.

The term “alkyoxyalkyl” as used herein by itself or as part of another group refers to an alkyl group substituted with an alkoxyl group. In some aspects, the alkyl is a C₁-C₄ alkyl and the alkoxy is a C₁-C₄ alkoxyl and the resulting alkoxyalkyl is thus referred to as a “C₁-C₄alkoxyC₁-C₄alkyl”.

The term “amino” as used herein by itself or as part of another group refers to —NH₂, which may be optionally substituted with one or two alkyl, two alkyl linked to form a ring, haloalkyl, (hydroxy)alkyl, (alkoxy)alkyl, (amino)alkyl, heteroalkyl, optionally substituted cycloalkyl, optionally substituted heterocyclo, optionally substituted aryl, optionally substituted heteroaryl, (aryl)alkyl, (cycloalkyl)alkyl, (heterocyclo)alkyl, or (heteroaryl)alkyl.

The term “heterocycylamino” as used herein by itself or as part of another group refers to an amino group substituted with a heterocyclyl group.

The term “aminoalkyl” as used herein by itself or as part of another group refers to an alkyl group substituted with an amino group. In some aspects, the alkyl is a C₁-C₄alkyl and the resulting aminoalkyl is thus referred to as an “aminoC₁-C₄alkyl”.

The term “hydroxyalkyl” as used herein by itself or as part of another group refers to an alkyl group substituted with a hydroxy group. In some aspects, the alkyl is a C₁-C₄alkyl and the resulting hydroxyalkyl is thus referred to as a “hydroxyC₁-C₄alkyl”.

The term “aminoalkylamino” as used herein by itself or as part of another group refers to an amino group substituted with an aminoalkyl group. In some aspects, the aminoalkylamino group is —NHCH₂CH₂NH₂.

The term “oxo” as used herein refers to an oxygen atom that is connected to a carbon atom by a double bond, i.e. to form a keto group.

The term “cycloalkyl” as used herein by itself or as part of another group refers to saturated and partially unsaturated, e.g., containing one or two double bonds, monocyclic, bicyclic, or tricyclic aliphatic hydrocarbons containing three to twelve carbon atoms, i.e., a C₃-C₁₂cycloalkyl, or the number of carbons designated, e.g., a C₃cycloalkyl such a cyclopropyl, a C₄cycloalkyl such as cyclobutyl, etc. In some aspects, the cycloalkyl is bicyclic, i.e., it has two rings. In some aspects, the cycloalkyl is monocyclic, i.e., it has one ring. In some aspects, the cycloalkyl is a C₃-C₈cycloalkyl. In some aspects, the cycloalkyl is a C₃₋₆cycloalkyl, i.e., cyclopropyl, cyclobutyl, cyclopentyl, or cyclohexyl. In some aspects, the cycloalkyl is a C₅cycloalkyl, i.e., cyclopentyl or cyclopentenyl. In some aspects, the cycloalkyl is a C₆cycloalkyl, i.e., cyclohexyl or cyclohexenyl. A cycloalkyl group containing one or two double bonds may also be referred to as a “cycloalkenyl” group.

The term “heterocyclyl” as used herein by itself or as part of another group refers to saturated and partially unsaturated, e.g., containing one or two double bonds, monocyclic, bicyclic, or tricyclic groups containing three to fourteen ring members, i.e., a 3- to 14-membered heterocyclyl, comprising one, two, three, or four heteroatoms. Each heteroatom is independently oxygen, sulfur, or nitrogen. The term heterocyclyl includes groups wherein one or more —CH₂— groups is replaced with one or more —C(═O)— groups, including cyclic ureido groups such as imidazolidinyl-2-one, cyclic amide groups such as piperidin-2-one or piperazin-2-one, and cyclic carbamate groups such as oxazolidinyl-2-one.

The term “heterocyclylaryl” as used herein by itself or as part of another group refers to an aryl group that is substituted with a heterocyclyl group.

The term “arylC₁-C₄alkyl” as used herein by itself or as part of another group refers to a C₁-C₄alkyl group substituted with an aryl group.

The term “heteroarylC₁-C₄alkyl” as used herein by itself or as part of another group refers to a C₁-C₄alkyl group substituted with a heteroaryl group.

The term “heterocyclylC₁-C₄alkyl” as used herein by itself or as part of another group refers to a C₁-C₄alkyl group substituted with a heterocyclyl group.

The term “cycloalkylC₁-C₄alkyl” as used herein by itself or as part of another group refers to a C₁-C₄alkyl group substituted with a cycloalkyl group.

The term “alkylsulfonyl” as used herein by itself or as part of another group refers to a sulfonyl group, i.e., —SO₂—, substituted by an alkyl group. A non-limiting exemplary alkylsulfonyl group is —SO₂CH₃.

The term “aryl” as used herein by itself or as part of another group refers to an aromatic ring system having six to fourteen carbon atoms, i.e., C₆-C₁₄ aryl. Non-limiting exemplary aryl groups include phenyl (abbreviated as “Ph”), naphthyl, phenanthryl, anthracyl, indenyl, azulenyl, biphenyl, biphenylenyl, and fluorenyl groups. In some aspects, the aryl group is phenyl.

The term “heteroaryl” as used herein by itself or as part of another group refers to monocyclic and bicyclic aromatic ring systems having five to fourteen ring members, i.e., a 5- to 14-membered heteroaryl, comprising one, two, three, or four heteroatoms. Each heteroatom is independently oxygen, sulfur, or nitrogen. In some aspects, the heteroaryl has three heteroatoms. In some aspects, the heteroaryl has two heteroatoms. In some aspects, the heteroaryl has one heteroatom. In some aspects, the heteroaryl is a 5- to 10-membered heteroaryl. In some aspects, the heteroaryl has 5 ring atoms, e.g., thienyl, a 5-membered heteroaryl having four carbon atoms and one sulfur atom. In some aspects, the heteroaryl has 6 ring atoms, e.g., pyridyl, a 6-membered heteroaryl having five carbon atoms and one nitrogen atom. Non-limiting exemplary heteroaryl groups include thienyl, benzo[b]thienyl, naphtho[2,3-b]thienyl, thianthrenyl, furyl, benzofuryl, pyranyl, isobenzofuranyl, benzoxazolyl, chromenyl, xanthenyl, 2H-pyrrolyl, pyrrolyl, imidazolyl, pyrazolyl, pyridyl, pyrazinyl, pyrimidinyl, pyridazinyl, isoindolyl, 3H-indolyl, indolyl, indazolyl, purinyl, isoquinolyl, quinolyl, phthalazinyl, naphthyridinyl, cinnolinyl, quinazolinyl, pteridinyl, 4aH-carbazolyl, carbazolyl, β-carbolinyl, phenanthridinyl, acridinyl, pyrimidinyl, phenanthrolinyl, phenazinyl, thiazolyl, isothiazolyl, phenothiazolyl, isoxazolyl, furazanyl, and phenoxazinyl. The term heteroaryl also includes N-oxides. A non-limiting exemplary N-oxide is pyridyl N-oxide.

The term “aminoheterocyclyl” as used herein by itself or as part of another group refers to a heterocyclyl group substituted with an optionally substituted amino group. Non-limiting exemplary aminoheterocyclyl groups include:

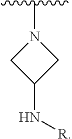

The chemical terms used herein may be combined to describe larger substituents. For a monovalent substituent, the substituent that is recited last in the combined term is the substituent that contains the attachment point. For example, an “arylC₁-C₄alkyl” group contains an attachment point on the alkyl group, whereas an “C₁-C₄alkylaryl” group contains an attachment point on the aryl group.

The present disclosure encompasses any of the disclosed compounds being isotopically-labelled (i.e., radiolabeled) by having one or more atoms replaced by an atom having a different atomic mass or mass number. Examples of isotopes that can be incorporated into the disclosed compounds include isotopes of hydrogen, carbon, nitrogen, oxygen, phosphorous, fluorine and chlorine, such as ¹H, ²H (or deuterium (D)), ³H, ¹¹C, ¹²C, ¹³C, ¹⁴C, ¹⁴N ¹⁵N, ¹⁸O, ¹⁷O, ¹⁶O ³¹P, ³²P, ³²S, ³³S, ³⁴S, ³⁵S, ³⁶S, ¹⁸F, ¹⁹F, ³⁵Cl, ³⁷Cl, and ³⁶Cl, respectively, e.g., ³H, ¹¹C, and ¹⁴C. In some aspects, provided is a composition wherein substantially all of the atoms at a position within the disclosed compound are replaced by an atom having a different atomic mass or mass number. In some aspects, provided is a composition wherein a portion of the atoms at a position within the disclosed compound are replaced, i.e., the disclosed compound is enriched at a position with an atom having a different atomic mass or mass number. Isotopically-labelled disclosed compounds can be prepared by methods known in the art. The present disclosure also encompasses any of the disclosed compounds wherein a quaternary carbon atom is replaced with a silicon atom.

As noted above, the compounds disclosed herein contain one or more asymmetric carbon atoms and may thus give rise to enantiomers, diastereomers, and other stereoisomeric forms. The present disclosure encompasses the use of all such possible forms, as well as their racemic and resolved forms and mixtures thereof. The individual enantiomers can be separated according to methods known in the art in view of the present disclosure. When the compounds described herein contain olefinic double bonds or other centers of geometric asymmetry, and unless specified otherwise, it is intended that they include both E and Z geometric isomers. All tautomers are also encompassed by the present disclosure.

As used herein, the term “stereoisomers” is a general term for all isomers of individual molecules that differ only in the orientation of their atoms in space. It includes enantiomers and isomers of compounds with more than one chiral center that are not mirror images of one another (diastereomers).

The term “chiral center” or “asymmetric carbon atom” refers to a carbon atom to which four different groups are attached.

The terms “enantiomer” and “enantiomeric” refer to a molecule that cannot be superimposed on its mirror image and hence is optically active wherein the enantiomer rotates the plane of polarized light in one direction and its mirror image compound rotates the plane of polarized light in the opposite direction.

The term “racemic” refers to a mixture of equal parts of enantiomers and which mixture is optically inactive. In some aspects, the compounds disclosed are racemic.

The term “absolute configuration” refers to the spatial arrangement of the atoms of a chiral molecular entity (or group) and its stereochemical description, e.g., R or S.

The stereochemical terms and conventions used in the specification are meant to be consistent with those described in Pure & Appl. Chem 68:2193 (1996), unless otherwise indicated.

The term “enantiomeric excess” or “ee” refers to a measure for how much of one enantiomer is present compared to the other. For a mixture of R and S enantiomers, the percent enantiomeric excess is defined as |R−S|*100, where R and S are the respective mole or weight fractions of enantiomers in a mixture such that R+S=1. With knowledge of the optical rotation of a chiral substance, the percent enantiomeric excess is defined as ([α]_{obs}/[α]ₘₐₓ)*100, where [α]_{obs} is the optical rotation of the mixture of enantiomers and [α]ₘₐₓ is the optical rotation of the pure enantiomer. Determination of enantiomeric excess is possible using a variety of analytical techniques, including NMR spectroscopy, chiral column chromatography or optical polarimetry.

The terms “administration,” “administering,” and grammatical variants thereof, refer to introducing a composition, such as a PDGFRα inhibitor of the present disclosure (e.g., small molecules or antibodies), into a subject via a pharmaceutically acceptable route. Any suitable route of administration can be used in administering the PDGFRα inhibitors described herein to a subject. Non-limiting examples of such routes of administration are provided elsewhere in the present disclosure.

The terms “antibody” and “antibodies” are terms of art and can be used interchangeably herein and refer to a molecule with an antigen binding site that specifically binds an antigen (e.g., a PDGFRα). The terms, as used herein, include whole antibodies and any antigen binding fragments (i.e., “antigen-binding portions”) or single chains thereof. Antigen-binding fragments or portions include, e.g., a Fab, a Fab₂, a Fab′, a F(ab′)₂, and an scFv. An “antibody” refers, in some aspects, to a glycoprotein comprising at least two heavy (H) chains and two light (L) chains inter-connected by disulfide bonds, or an antigen-binding portion thereof. In some aspects, an “antibody” refers to a single chain antibody comprising a single variable domain, e.g., VHH domain.

“Antibody” includes, by way of example, both naturally-occurring and non-naturally-occurring antibodies; monoclonal and polyclonal antibodies; chimeric and humanized antibodies; human and non-human antibodies; wholly synthetic antibodies; single chain antibodies; monospecific antibodies; multispecific antibodies (including bispecific antibodies); tetrameric antibodies comprising two heavy chain and two light chain molecules; an antibody light chain monomer; an antibody heavy chain monomer; an antibody light chain dimer, an antibody heavy chain dimer; an antibody light chain-antibody heavy chain pair; intrabodies; heteroconjugate antibodies; monovalent antibodies; camelized antibodies; affibodies; anti-idiotypic (anti-Id) antibodies (including, e.g., anti-anti-Id antibodies), and single-domain antibodies (sdAbs), which include binding molecules consisting of a single monomeric variable antibody domain that are fully capable of antigen binding (e.g., a VH domain or a VL domain). Harmen M. M, and Haard H. J. Appl Microbiol Biotechnol. 77(1): 13-22 (2007)). In some aspects, the antibody is a monoclonal antibody. In some aspects, the antibody is a human antibody. In some aspects, the antibody is a humanized antibody.

The term “central nervous system” or “CNS” refers to a complex of nerve tissues that control the various activities of the body (e.g., voluntary and involuntary movements) and the mind (e.g., thoughts, perceptions, and emotions). The CNS generally consists of the brain and the spinal cord.

As used herein, the term “demyelinating disease” refers to any disorder of the nervous system in which there is reduced myelination, including disorders in which insufficient or dysfunctional myelin (e.g., hypomyelination) is generated during development or disorders in which the myelin sheath of neurons is damaged. “Myelin” and “Myelin sheath” refer to the specialized membrane formed by oligodendrocytes that insulates the axons of neurons. The insulation provided by the myelin sheath helps to increase the rate of transmission of nerve signals along the axon, thereby promoting timely and energetically efficient neuronal signaling. Myelin also provides metabolic support of the axons, maintaining their health and survival. In some aspects, the neuronal axon of a subject suffering from or at risk of developing a demyelinating disease is completely demyelinated. In some aspects, the neuronal axon of a subject suffering from or at risk of developing a demyelinating disease is partially demyelinated (e.g., at least 5%, at least 10%, at least 20%, at least 30%, at least 40%, at least 50%, at least 60%, at least 70%, at least 80%, or at least 95% compared to a reference neuronal axon that is fully myelinated). Accordingly, unless indicated otherwise, the term “demyelination” comprises any of the following: complete hypomyelination, complete demyelination, partial hypomyelination, partial demyelination, and combinations thereof. In some aspects, because of the partial and/or complete demyelination, the demyelinating diseases described herein are associated with impaired conduction of nerve signals or axonal or neuronal survival, which can, in turn, cause deficiencies in sensation, movement, cognition, or other functions depending on which neurons are affected. Non-limiting examples of demyelinating diseases are provided elsewhere in the present disclosure. In general, the term “under myelination” refers to a cell, tissue, or subject that lacks normal levels of myelination (e.g., level of myelination observed in a corresponding cell, tissue, or subject who does not have a demyelinating disease), regardless of etiology.

As used herein, the term “hypomyelination” refers to a deficiency in myelin for any reason (e.g., body is unable to produce myelin at normal levels). Unless indicated otherwise, hypomyelination includes demyelination (related to myelin destruction) and dysmyelination (related to abnormal myelin deposition). Accordingly, hypomyelination includes diseases in which insufficient myelin is generated during development as well as diseases associated with demyelination and/or dysmyelination. In some aspects, compared to a reference (e.g., corresponding tissue, e.g., brain, of a subject who does not suffer from a demyelinating disease), there is hypomyelination of all (i.e., “complete hypomyelination”) or a portion of the nervous system (i.e., “partial hypomyelination”), for example the brain (e.g., hypomyelination in white matter and/or gray matter), of at least 5%, at least 10%, at least 20%, at least 30%, at least 40%, at least 50%, at least 60%, at least 70%, at least 80%, or at least 95%.

As used herein, the term “neuron” includes electrically excitable cells that process and transmit information through electrical and chemical signals. Neurons are major components of the brain and spinal cord of the CNS, and of the ganglia of the peripheral nervous system (PNS) and can connect to each other to form neural networks. A typical neuron is composed of a cell body (soma), dendrites, and an axon. The “soma” (the cell body) of a neuron contains the nucleus. The “dendrites” of a neuron are cellular extensions generally having many branches, where the majority of input to the neuron occurs. The “axon” (also referred to herein as “neuronal axon”) extends from the soma and carries nerve signals away from the soma and certain types of information back to the soma.

As used herein, the term “oligodendrocyte progenitor cells” or “OPCs” (also known in the art as “oligodendrocyte precursor cells,” “polydendrocytes,” “NG2 cells,” and “O-2A cells”) refer to a subtype of glial cells in the central nervous system. They are precursors to “oligodendrocytes,” (also known as “oligodendroglia”) which are responsible for generating the myelin sheath that wraps around axons, providing insulation, aiding electrical conduction, and providing metabolic support. Nerve impulses can travel up to 200 times faster along a myelinated neuron compared to an unmyelinated axon. OPCs and immature oligodendrocytes are generally positive for the following markers: A2B5, neuron-glial antigen 2 (NG2), and PDGFRα. Other suitable markers that can be used are known in the art.

As used herein, the term “remyelination” (or derivatives thereof) refers to generation of new myelin sheaths around demyelinated (e.g., including hypomyelinated) axons. The remyelination process involves the differentiation of OPCs into oligodendrocytes that generate functional myelin sheaths around demyelinated axons. Remyelination of the axons can restore action potential conduction properties to axons, and thereby, promote and/or improve neurological function. Further, remyelination can provide metabolic support to axons, preventing their damage or loss. In the context of the present application, unless otherwise specified, “remyelination” refers to any aspect of a process that can result in remyelination. For example, in some aspects, “remyelination” comprises the migration or colonization of OPCs to sites of demyelinated axons. In some aspects, “remyelination” comprises the differentiation of OPCs into oligodendrocytes. In some aspects, “remyelination” comprises the generation of myelin sheaths by oligodendrocytes around demyelinated axons. In some aspects, the term “remyelination” comprises any combination of the following: (i) migration or colonization of OPCs to sites of demyelinated axons; (ii) differentiation of OPCs into oligodendrocytes; and (iii) generation of myelin sheaths by oligodendrocytes around demyelinated (e.g., including hypomyelinated) axons.

As used herein, the term “restore” (and derivatives thereof) comprises both complete restoration and partial restoration. For example, in some aspects, remyelinating a demyelinated axon restores conduction properties to the axon, such that the conduction properties are the same as those of the axon prior to the demyelination (i.e., complete restoration). In some aspects, remyelinating a demyelinated axon restores conduction properties to the axon, where the conduction properties are improved but not the same as those of the axon prior to the demyelination (i.e., partial restoration). In some aspects, remyelination provides metabolic support to axons, preventing their damage or loss.

As used herein, the term “promoting” refers to the ability of an agent (e.g., PDGFRα inhibitor described herein) to induce or increase a particular result (e.g., remyelination of a demyelinated axon). In some aspects, the term comprises both inducing and increasing a particular result.

As used herein, the term “subject” refers to any animal subject including a human, a laboratory animal (e.g., a non-human primate, rat, and mouse), livestock (e.g., cow, sheep, goat, pig, turkey, and chicken), and household pets (e.g., dog, cat, and rodent).

The terms “treat,” “treating,” and “treatment,” as used herein, refer to any type of intervention or process performed on, or administering an active agent (e.g., PDGFRα inhibitor described herein) to, the subject with the objective of reversing, alleviating, ameliorating, inhibiting, or slowing down; or preventing the progression, development, severity or recurrence of a symptom, complication, condition or one or more biochemical indicia associated with a disease, or enhancing overall survival. As described herein, in some aspects, treatment can be of a subject having a disease (e.g., exhibiting one or more symptoms associated with the disease). In some aspects, treatment can be of a subject with some degree of demyelination but not yet exhibiting any symptoms associated with the disease. In the context of such subjects, administering a PDGFRα inhibitor of the present disclosure can help delay or prevent the onset of symptoms associated with the disease.

The term “effective dose” or “effective amount” is defined as an amount sufficient to achieve or at least partially achieve a desired effect (e.g., induce remyelination of a demyelinated neuronal axon). A “therapeutically effective amount” or “therapeutically effective dosage” of a drug or therapeutic agent (e.g., PDGFRα inhibitor) is any amount of the drug that, when used alone or in combination with another therapeutic agent, promotes disease regression as evidenced by a decrease in severity of disease symptoms, an increase in frequency and duration of disease symptom-free periods, a prevention of impairment or disability due to the disease affliction, or a reduction in disease progression. A therapeutically effective amount or dosage of a drug includes a “prophylactically effective amount” or a “prophylactically effective dosage,” which is any amount of the drug that, when administered alone or in combination with another therapeutic agent to a subject at risk of developing a disease or of suffering a recurrence of disease, inhibits the development or recurrence of the disease. The ability of a therapeutic agent to promote disease reversal or inhibit the development, progression or recurrence of the disease can be evaluated using a variety of methods known to the skilled practitioner, such as in human subjects during clinical trials, in animal model systems predictive of efficacy in humans, or by assaying the activity of the agent in in vitro assays.

As used herein, the term “dosing interval” refers to the amount of time that elapses between multiple (e.g., 2 or more) doses of a PDGFRα inhibitor described herein. Not to be bound by any one theory, in some aspects, a PDGFRα inhibitor provided herein exerts its therapeutic effect by inducing the differentiation of oligodendrocyte progenitor cells (OPCs) into oligodendrocytes. Accordingly, in some aspects, for a PDGFRα inhibitor to have a therapeutic effect in a subject (e.g., by inducing OPC differentiation), it is necessary that the subject has a pool of OPCs which a PDGFRα inhibitor described herein can act upon. As demonstrated herein (see, e.g., Example 246), administering a PDGFRα inhibitor too frequently to a subject can be counterproductive in treating a demyelinating disease because the OPC population would not have sufficient time to repopulate the brain. Accordingly, in some aspects, a dosing interval that is suitable for the present disclosure is the amount of time required for a subject's OPC population to have sufficiently recovered after the administration of an initial dose of a PDGFRα inhibitor provided herein, such that the administration of a second (or additional dose) of the PDGFRα inhibitor would have a therapeutic effect (e.g., such as those described herein, e.g., increases the number of differentiated oligodendrocytes) in the subject. As is apparent from the present disclosure, in some aspects, a PDGFRα inhibitor described herein is administered to a subject at a dosing interval, wherein the dosing interval is the amount of time required, after the administration of an initial dose of the PDGFRα inhibitor, for the subject's OPC population (e.g., in size) to be at least 10%, at least 20%, at least 30%, at least 40%, at least 50%, at least 60%, at least 70%, at least 80%, at least 90%, or at least 100% to that of a reference OPC population (e.g., the subject's OPC population prior to the initial administration of the PDGFRα inhibitor). In some aspects, a suitable dosing interval for the present disclosure comprises the amount of time required, after an initial dose of the PDGFRα inhibitor, for the plasma level of the PDGFRα inhibitor to reach less than 50%, less than 40%, less than 30%, less than 20%, less than 10%, or less than 5%, as compared to a reference (e.g., plasma level of the PDGFRα inhibitor in a corresponding subject immediately after, e.g., about 4 hours after, the administration of the initial dose of the PDGFRα inhibitor), a suitable dosing interval is the time between administering the first dose of the PDGFRα inhibitor and the subject's plasma level of the PDGFRα inhibitor reaching a reduced level as compared to the reference. In some aspects, the additional dose of the PDGFRα inhibitor is administered to the subject when the subject's plasma level has decreased by at least 10%, at least 20%, at least 30%, at least 40%, at least 50%, at least 60%, at least 70%, at least 80%, at least 90%, or about 100% as compared to the reference.

As used herein, the terms “binds,” “recognizes,” “targets are analogous terms and refer to molecules (e.g., PDGFRα inhibitors described herein) that can bind and/or target a particular region of a protein (e.g., PDGFRα), as such binding and/or targeting is understood by one skilled in the art. For instance, as described herein, in some aspects, a PDGFRα inhibitor is an antibody, which can bind to the extracellular region of PDGFRα. In some aspects, the antibody interferes with the binding of PDGFRα to its ligand, for example it reduces or abrogates the binding of PDGFRα to PDGF. In some aspects, the antibody directly reduces the binding between PDGFRα and its ligand. The antibody may thus act as a competitive inhibitor of the PDGFRα-PDGF interaction. In some aspects, the antibody interferes with the binding of the ligand and/or with PDGFRα activation in other ways, for example by preventing PDGFRα dimerization and/or by stabilizing an inactive form of PDGFRα. The antibody may be a non-competitive inhibitor of the PDGFRα-PDGF interaction. In some aspects, a PDGFRα inhibitor is a small molecule that can target and inhibit the kinase portion of PDGFRα (e.g., by binding to the ATP binding site, by allosterically interfering with PDGFRα kinase activity, or both), and thereby, inhibit its activity. Unless indicated otherwise, the above terms (i.e., binds, recognizes, and targets) are used interchangeably and comprises any binding or targeting of PDGFRα, such that its activity is reduced and/or inhibited (e.g., binding of an antagonistic anti-PDGFRα antibody to the extracellular region of PDGFRα, binding of a small molecule to an ATP binding site of PDGFRα, binding of a small molecule to a substrate binding site of PDGFRα, allosterically interfering with PDGFRα kinase activity, or a combination thereof).

“Potency” is an expression of the activity of a drug in terms of the amount or concentration of the drug that achieves a desired effect. Accordingly, functional assays, such as those described herein, can be used to identify compounds useful for the present disclosure.

Molecules (e.g., PDGFRα inhibitors, such as anti-PDGFRα antibodies described herein) that “compete with another protein or compound for binding to a target” refers to molecules that inhibit (partially or completely) the binding of the other protein (e.g., naturally existing PDGFRα ligand) to the target (e.g., PDGFRα). Whether two compounds compete with each other for binding to a target, i.e., whether and to what extent a PDGFRα inhibitor described herein inhibits the binding of the naturally existing ligand to a PDGFRα, can be determined using known competition experiments. In some aspects, a PDGFRα inhibitor described herein competes with, and inhibits the binding of the naturally existing ligand to PDGFRα by at least 10%, at least 20%, at least 30%, at least 40%, at least 50%, at least 60%, at least 70%, at least 80%, at least 90% or 100%. Competition assays can be conducted as described herein or, for example, in Ed Harlow and David Lane, Cold Spring Harb. Protoc.; 2006; doi: 10.1101/pdb.prot4277 or in Chapter 11 of “Using Antibodies” by Ed Harlow and David Lane, Cold Spring Harbor Laboratory Press, Cold Spring Harbor, NY, USA 1999.

Examples of other competitive binding assays that can be used with the present disclosure include: solid phase direct or indirect radioimmunoassay (RIA), solid phase direct or indirect enzyme immunoassay (EIA), sandwich competition assay (see Stahli et al., Methods in Enzymology 9:242 (1983)); solid phase direct biotin-avidin EIA (see Kirkland et al., J. Immunol. 137:3614 (1986)); solid phase direct labeled assay, solid phase direct labeled sandwich assay (see Harlow and Lane, Antibodies: A Laboratory Manual, Cold Spring Harbor Press (1988)); solid phase direct label RIA using 1-125 label (see Morel et al., Mol. Immunol. 25(1): 7 (1988)); solid phase direct biotin-avidin EIA (Cheung et al., Virology 176:546 (1990)); and direct labeled RIA. (Moldenhauer et al., Scand. J. Immunol. 32:77 (1990)).

As further described elsewhere in the present disclosure, in some aspects, the antagonistic activity of a PDGFRα inhibitor described herein does not depend on interfering with the binding of another protein or compound to PDGFRα. Another non-limiting example includes small molecules that can bind to PDGFRα and inhibit (partially or completely) binding of ATP or substrate, or otherwise inhibit PDGFRα activity. In some aspects, a PDGFRα inhibitor described herein can inhibit or reduce PDGFRα activity via allosteric inhibition. In some aspects, a PDGFRα inhibitor can reduce protein levels of PDGFRα by reducing synthesis or enhancing degradation. While exemplary mechanisms of actions are provided, it will be apparent from the present disclosure that the PDGFRα inhibitors described herein are not intended to be limited to such mechanisms.

A “polypeptide” refers to a chain comprising at least two consecutively linked amino acid residues, with no upper limit on the length of the chain. One or more amino acid residues in the protein can contain a modification such as, but not limited to, glycosylation, phosphorylation or disulfide bond formation. A “protein” can comprise one or more polypeptides.

As used herein, the terms “ug” and “uM” are used interchangeably with “ug” and “UM,” respectively.

Various aspects described herein are described in further detail in the following subsections. The various aspects and embodiments of the invention disclosed throughout the specification can optionally be combined. For instance, in some embodiments any of the disclosed compounds can be used in any of the disclosed methods and uses.

### II. PDGFRα Inhibitors

Disclosed herein are compounds that can inhibit PDGFRα activity and/or elicit one or more activities associated with inhibition of the receptor. PDGFRα can bind with high affinity to several PDGF isoforms (i.e., PDGF-A, PDGF-B, PDGF-C, and PDGF-D). As used herein, references to inhibition of PDGFRα binding to its ligand refer to inhibition of binding of one or more of the PDGF isoforms that bind to PDGFRα. Preferably, the binding of all isoforms of PDGF that bind to PDGFRα is inhibited. The binding of the platelet-derived growth factor (PDGF) isoforms to PDGFRα affects various cell signaling pathways, such as those involved in cell proliferation and differentiation. Accordingly, PDGFRα activity is critical for both the proper development and long-term maintenance of certain tissues and organs. For example, animals globally lacking a functional PDGFRA gene die at birth or soon thereafter due to various developmental defects (e.g., cardiac malformations). Bax et al., Dev. Dyn. 239 (8): 2307-2317 (August 2010). Conditional PDGFRα knockout in OPCs during animal development results in severe hypomyelination and the animals die soon after birth Hamashima et al., Neuroscience 436:11-26 (June 2020)

In humans, the gene encoding PDGFRα (i.e., PDGFRA) is located on chromosome 4 (for example, nucleotides 54,229,127-54,298,245 of GenBank Accession Number NC_000004.12; plus strand orientation). In addition to those provided above, other synonyms of PDGFRα are known and include: “PDGFRa,” “PDGFR2,” “alpha-type platelet-derived growth factor receptor,” “platelet-derived growth factor receptor A,” “platelet-derived growth factor receptor alpha,” “platelet-derived growth factor receptor 2,” “CD140a,” and “CD140 antigen-like family member A.” As used herein, the term “PDGFRα” includes any variants or isoforms of PDGFRα that are naturally expressed by cells.

Despite its importance in various biological processes, the present disclosure describes the discovery that inhibiting or reducing PDGFRα activity, for example, with one or more of the compounds described herein can have certain biological effects that can be useful for treating demyelinating diseases, such as those described herein. As demonstrated herein, in some aspects, inhibiting or reducing PDGFRα activity with the PDGFRα inhibitors described herein can promote oligodendrocyte differentiation and induce myelin formation. In some aspects, the PDGFRα inhibitors of the present disclosure are capable of (and in some embodiments used for) promoting remyelination of demyelinated neuronal axons, which can be useful in the treatment of various demyelinating diseases (e.g., multiple sclerosis). As used herein, the terms “PDGFRα inhibitors” and “PDGFRα antagonists” are used interchangeably and refer to any compound (e.g., a small molecule or antibody) that is capable of (and in some embodiments used for) reducing and/or inhibiting PDGFRα activity (e.g., by any of the exemplary mechanisms or methods described herein). In some aspects, a PDGFRα inhibitor provided herein can inhibit a tyrosine kinase activity of a PDGFRα. In some aspects, a PDGFRα inhibitor provided herein can inhibit any other activity of a PDGFRα. Where the term “capable of” is used herein to describe certain features of a PDGFRα inhibitor, the term means that the PDGFRα inhibitor can (i.e., has the ability to) exhibit such features, e.g., under appropriate conditions. Unless indicated otherwise, the term does not mean that the PDGFRα inhibitor always exhibits such features, e.g., when administered to a subject with impaired oligodendrocyte progenitor cell population. The term “capable of” and “can” are used interchangeably in the present application.

In some aspects, a PDGFRα inhibitor (e.g., small molecule described herein) inhibits the kinase activity of PDGFRα. As used herein, unless indicated otherwise, the term “inhibit” (and derivatives thereof) comprises both complete inhibition and partial inhibition (e.g., reduced kinase activity). Accordingly, in some aspects, when an OPC (expressing PDGFRα) interacts with a PDGFRα inhibitor described herein (e.g., small molecule), the PDGFRα kinase activity is reduced by at least 5%, at least 10%, at least 15%, at least 20%, at least 25%, at least 30%, at least 35%, at least 40%, at least 45%, at least 50%, at least 60%, at least 70%, at least 80%, at least 90% or more (for example, at least 50% or more), compared to PDGFRα kinase activity in a corresponding OPC that was not contacted with the PDGFRα inhibitor described herein (e.g., contacted with a vehicle control). In some aspects, the kinase activity is completely inhibited. As is apparent from the present disclosure, the inhibition (partial or complete) of PDGFRα kinase activity in an OPC can promote the differentiation of the OPC into a myelinating oligodendrocyte. Inhibition of PDGFRα kinase activity can be assayed using methods known in the art. In some aspects, the inhibition of PDGFRα kinase activity can be determined using the in vitro differentiation assay described in Example 240 and in section VI of the present application. In some aspects, the inhibition of PDGFRα kinase activity can be assessed using a cuprizone model for demyelination, such as that described in Torkildsen et al., Acta Neurol Scand Suppl 188:72-6 (2008) or in Example 243 of the present disclosure. In some aspects, the inhibition of PDGFRα kinase activity is determined using an enzymatic PDGFRα kinase assay, such as that described in Example 239 of the present disclosure. In some aspects, the inhibition of PDGFRα kinase activity can be determined using an in vivo OPC differentiation assay, such as that described in Example 245.

In some aspects, a PDGFRα inhibitor described herein (e.g., antibody) can specifically bind to a PDGFRα (e.g., to an extracellular region of PDGFRα). In some aspects, the antibody interferes with the binding of the PDGFRα to its natural ligand. Not to be bound by any one theory, in some aspects, by interfering with the binding of PDGFRα to its ligand, a PDGFRα inhibitor can inhibit (completely or partially) PDGFRα activity of an OPC, and thereby, induce the differentiation of the OPC into a myelinating oligodendrocyte. In some aspects, the interaction of the inhibitor with PDGFRα does not significantly affect binding of PDGFRα to a PDGF monomer or PDGF dimer.

In some aspects, a PDGFRα inhibitor useful for the present disclosure comprises a polypeptide, small molecule, antibody, gene editing tool, or any combination thereof. Additional aspects of such PDGFRα inhibitors are provided elsewhere in the present disclosure.

As described herein, in some aspects, a PDGFRα inhibitor useful for the present disclosure comprises one or more of the following properties: (1) capable of (and in some embodiments used for) promoting (e.g., inducing and/or enhancing) the differentiation of an OPC into an oligodendrocyte (in vitro, in vivo, or both); (2) capable of (and in some embodiments used for) promoting (e.g., inducing and/or enhancing) the expression of a protein associated with oligodendrocyte differentiation and/or myelination (e.g., G-protein coupled receptor 17 (GPR17), myelin basic protein (MBP), ASPA, GST-pi, CC1, myelin oligodendrocyte glycoprotein (MOG), oligodendrocyte-specific protein/claudin-11, CNPase, or a combination thereof); (3) capable of (and in some embodiments used for) promoting (e.g., inducing and/or enhancing) the myelination of a neuronal axon; (4) capable of (and in some embodiments used for) promoting (e.g., inducing and/or enhancing) the remyelination of a demyelinated neuronal axon; (5) capable of (and in some embodiments used for) inhibiting a PDGFRα kinase activity (e.g., IC₅₀ of less than 10,000 nM, as measured using a Promega kinase assay; (e.g., described in Example 239); (6) capable of (and in some embodiments used for) penetrating the brain (e.g., following systemic administration) at a sufficient level to support activity in the CNS; and (7) any combination thereof. Non-limiting examples of additional properties are provided elsewhere in the present disclosure.

1. Promoting the Differentiation of an OPC into an Oligodendrocyte

In some aspects, PDGFRα inhibitors described herein are capable of (and in some embodiments used for) promoting (e.g., inducing and/or enhancing) the differentiation of an OPC into an oligodendrocyte when the OPC is contacted (e.g., in vivo or ex vivo) with the PDGFRα inhibitor. In some aspects, after the contacting, the OPC undergoes phenotypic and functional changes, resulting in its differentiation into a myelinating oligodendrocyte. In some aspects, promoting the differentiation of an OPC into an oligodendrocyte results in an increase in the amount (e.g., percentage and/or number) of oligodendrocytes, e.g., within the CNS of a subject that received an administration of the PDGFRα inhibitor. Accordingly, in some aspects, a PDGFRα inhibitor of the present disclosure can increase the number of oligodendrocytes (e.g., within the CNS of a subject that received an administration of the PDGFRα inhibitor) by at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 25-fold, at least 30-fold, at least 35-fold, at least 40-fold, at least 45-fold, or at least 50-fold compared to a reference (e.g., the number of oligodendrocytes in the subject prior to the PDGFRα inhibitor administration; and/or the number of oligodendrocytes in a corresponding subject that did not receive an administration of the PDGFRα inhibitor). Unless indicated otherwise, the term “number” refers to both absolute number as well as percentage. Not to be bound by any one theory, in some aspects, an increase in the number of oligodendrocytes results in increased myelination of axons (e.g., hypomyelinated neuronal axons), remyelination of demyelinated neuronal axons, or both. In some aspects, an increase in the number of oligodendrocytes can be detected by visualizing and/or quantifying the expression of a marker associated with oligodendrocyte differentiation and/or myelination. In some aspects, an increase in the number of oligodendrocytes can be detected by visualizing and/or quantifying the expression of G-protein coupled receptor 17 (GPR17) ASPA, GST-pi, CC1, or a combination thereof. In some aspects, an increase in myelin can be detected by visualizing and/or quantifying the expression of myelin basic protein (MBP) Myelin Oligodendrocyte Glycoprotein (MOG), Oligodendrocyte Specific Protein/Claudin-11, CNPase, or any combination thereof. In some aspects, more than one marker can be used. For example, in some aspects, an increase in the number of oligodendrocytes can be detected by visualizing and/or quantifying the expression of G-protein coupled receptor 17 (GPR17) and myelin basic protein (MBP). In some aspects, marker(s) are visualized by immunohistochemistry. In some aspects, marker(s) are quantitated via automated image analysis from tissue sections. An increase can be detected compared to a control sample that has not been contacted or treated with the PDGFRα inhibitor.

As is apparent from the present disclosure, in some aspects, a PDGFRα inhibitor of the present disclosure comprises any agent known in the art (e.g., polypeptide, small molecule, antibody, or gene editing tool) that can target PDGFRα and promote the differentiation of an OPC into an oligodendrocyte. In some aspects, a PDGFRα inhibitor useful for the present disclosure comprises a compound of formula (I) that is capable of (and in some embodiments used for) promoting the differentiation of an OPC into an oligodendrocyte when contacted (e.g., in vivo, in vitro, or both) with the OPC. In some aspects, a PDGFRα inhibitor of the present disclosure comprises a compound of formula (II) that is capable of (and in some embodiments used for) promoting the differentiation of an OPC into an oligodendrocyte when contacted (e.g., in vivo, in vitro, or both) with the OPC. In some aspects, a PDGFRα inhibitor of the present disclosure comprises a compound of formula (III) that is capable of (and in some embodiments used for) promoting the differentiation of an OPC into an oligodendrocyte when contacted (e.g., in vivo, in vitro, or both) with the OPC. In some aspects, a PDGFRα inhibitor of the present disclosure comprises a compound of formula (IV) that is capable of (and in some embodiments used for) promoting the differentiation of an OPC into an oligodendrocyte when contacted (e.g., in vivo, in vitro, or both) with the OPC. In some aspects, a PDGFRα inhibitor comprises an antagonistic anti-PDGFRα antibody that is capable of (and in some embodiments used for) promoting the differentiation of an OPC into an oligodendrocyte when contacted with the OPC. Additional disclosure relating to such PDGFRα inhibitors are provided throughout the present application.

2. Promoting the Expression of a Protein Associated with Oligodendrocyte Differentiation and or Myelination

As demonstrated herein (see, e.g., Example 239), in some aspects, PDGFRα inhibitors that are useful for the present disclosure are capable of (and in some embodiments used for) promoting the expression of one or more proteins associated with oligodendrocyte differentiation and/or myelination. Non-limiting examples of such proteins include: GPR17, MBP, ASPA, GST-pi, CC1, MOG, oligodendrocyte-specific protein/claudin-11, CNPase, or a combination thereof. For example, in some aspects, a PDGFRα inhibitor described herein is capable of (and in some embodiments used for) promoting (e.g., inducing, enhancing, and/or increasing) the expression of GPR17 (i.e., a differentiation marker) in a nervous system cell (e.g., OPC). In some aspects, a PDGFRα inhibitor is capable of (and in some embodiments used for) promoting (e.g., inducing and/or enhancing) the expression of MBP in a nervous system cell (e.g., oligodendrocyte). In some aspects, a PDGFRα inhibitor is capable of (and in some embodiments used for) promoting the expression of both GPR17 and MBP.

Accordingly, in some aspects, when an OPC is contacted with a PDGFRα inhibitor described herein, the expression of one or more proteins associated with oligodendrocyte differentiation and/or myelination (e.g., GPR17 and/or MBP) is increased in the cell by at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 25-fold, at least 30-fold, at least 35-fold, at least 40-fold, at least 45-fold, or at least 50-fold (for example, at least 3-fold) compared to a reference (e.g., expression of the one or more proteins in a corresponding cell that was not contacted with the PDGFRα inhibitor), e.g., as measured in an in vitro or in vivo assay. Not to be bound by any one theory, in some aspects, an increased expression of the one or more proteins associated with oligodendrocyte differentiation and/or myelination (e.g., GPR17 and/or MBP) results in increased amount (e.g., percentage and/or number) of oligodendrocytes, which can in turn increase the myelination of neuronal axons (e.g., hypomyelinated), the remyelination of demyelinated neuronal axons, or both.

In some aspects, a PDGFRα inhibitor useful for the present disclosure includes any agent known in the art (e.g., polypeptide, small molecule, antibody, or gene editing tool) that can target PDGFRα and promote the expression of one or more proteins associated with oligodendrocyte differentiation and/or myelination (e.g., GPR17 and/or MBP).

In some aspects, a PDGFRα inhibitor that can be used with the present disclosure comprises a compound of formula (I) that is capable of (and in some embodiments used for) promoting the expression of one or more proteins associated with oligodendrocyte differentiation and/or myelination (e.g., GPR17 and/or MBP) in a nervous system cell. In some aspects, the PDGFRα inhibitor comprises a compound of formula (I) that is capable of (and in some embodiments used for) promoting both the expression of one or more proteins associated with oligodendrocyte differentiation and/or myelination (e.g., GPR17 and/or MBP) and the differentiation of an OPC into an oligodendrocyte. In some aspects, a PDGFRα inhibitor of the present disclosure comprises a compound of formula (II) that is capable of (and in some embodiments used for) promoting the expression of one or more proteins associated with oligodendrocyte differentiation and/or myelination (e.g., GPR17 and/or MBP) in a nervous system cell. In some aspects, the PDGFRα inhibitor comprises a compound of formula (II) that is capable of (and in some embodiments used for) promoting both the expression of one or more proteins associated with oligodendrocyte differentiation and/or myelination (e.g., GPR17 and/or MBP) and the differentiation of an OPC into an oligodendrocyte. In some aspects, a PDGFRα inhibitor of the present disclosure comprises a compound of formula (III) that is capable of (and in some embodiments used for) promoting the expression of one or more proteins associated with oligodendrocyte differentiation and/or myelination (e.g., GPR17 and/or MBP) in a nervous system cell. In some aspects, the PDGFRα inhibitor comprises a compound of formula (III) that is capable of promoting both the expression of one or more proteins associated with oligodendrocyte differentiation and/or myelination (e.g., GPR17 and/or MBP) and the differentiation of an OPC into an oligodendrocyte. In some aspects, a PDGFRα inhibitor of the present disclosure comprises a compound of formula (IV) that is capable of promoting the expression of one or more proteins associated with oligodendrocyte differentiation and/or myelination (e.g., GPR17 and/or MBP) in a nervous system cell. In some aspects, the PDGFRα inhibitor comprises a compound of formula (IV) that is capable of (and in some embodiments used for) promoting both the expression of one or more proteins associated with oligodendrocyte differentiation and/or myelination (e.g., GPR17 and/or MBP) and the differentiation of an OPC into an oligodendrocyte. In some aspects, a PDGFRα inhibitor comprises an antagonistic anti-PDGFRα antibody that is capable of (and in some embodiments used for) promoting the expression of one or more proteins associated with oligodendrocyte differentiation and/or myelination (e.g., GPR17 and/or MBP) in a nervous system cell. In some aspects, a PDGFRα inhibitor described herein comprises an antagonistic anti-PDGFRα antibody that is capable of (and in some embodiments used for) promoting both the expression of one or more proteins associated with oligodendrocyte differentiation and/or myelination (e.g., GPR17 and/or MBP) and the differentiation of an OPC into an oligodendrocyte.

### 3. Promoting the Myelination of a Neuronal Axon

As is apparent from the present disclosure, an important property of the PDGFRα inhibitors described herein is that they are able to promote the myelination of a neuronal axon (also referred to herein simply as “axon”). As further described elsewhere in the present disclosure, in some aspects, by promoting the myelination of an axon, the PDGFRα inhibitors described herein are capable of (and in some embodiments used for) improving nervous system cell (e.g., neuron) function and thereby, treat a disease or disorder described herein. Accordingly, in some aspects, when contacted (e.g., in vivo, in vitro, or both) with a PDGFRα inhibitor described herein, the number of neurons with axons that are myelinated is increased compared to a reference (e.g., corresponding number of neurons that are not contacted with the PDGFRα inhibitor). In some aspects, compared to the reference, the amount of neurons with axons that are myelinated is increased by at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 25-fold, at least 30-fold, at least 35-fold, at least 40-fold, at least 45-fold, or at least 50-fold (for example, at least 10-fold). The increase in the number of neurons with axons that are myelinated can be determined using any suitable methods known in the art or described herein. In some aspects, the increase in the number of neurons with axons that are myelinated can be determined by visualizing and/or quantifying the expression of a marker associated with myelinated neurons. For example, in some aspects, the marker associated with myelinated neurons comprises myelin basic protein (MBP), myelin oligodendrocyte glycoprotein (MOG), oligodendrocyte-specific protein/claudin-11, CNPase, or a combination thereof. In some aspects, the increase in the number of neurons with axons that are myelinated can be assessed using a cuprizone model, such as that described in Torkildsen et al., Acta Neurol Scand Suppl 188:72-6 (2008) or in Example 243 of the present disclosure. In some aspects, the increase in myelination is assessed using a modification of the cuprizone model (such as that described herein) in which animals are pre-treated with cuprizone for at least 4 months, at least 5 months, at least 6 months, at least 7 months, at least 8 months, at least 9 months, at least 10 months, at least 11 months, at least 12 months, at least 13 months, at least 14 months, at least 15 months, at least 16 months, or at least 17 months. In some aspects, the animals are pre-treated with cuprizone for at least 8 months. In some aspects, the animals are pre-treated with cuprizone for between 8-17 months.

In some aspects, a PDGFRα inhibitor useful for the present disclosure includes any agent known in the art (e.g., polypeptide, small molecule, antibody, or gene editing tool) that can target PDGFRα and promote the myelination of an axon.

In some aspects, a PDGFRα inhibitor described herein comprises a compound of formula (I) that is capable of (and in some embodiments used for) promoting the myelination of axons. In some aspects, a PDGFRα inhibitor comprises a compound of formula (I) that is capable of (and in some embodiments used for) both promoting the differentiation of an OPC into an oligodendrocyte and promoting the myelination of an axon. In some aspects, a PDGFRα inhibitor comprises a compound of formula (I) that is capable of both promoting the myelination of an axon and the expression of one or more proteins associated with oligodendrocyte differentiation and/or myelination (e.g., GPR17 and/or MBP). In some aspects, a PDGFRα inhibitor comprises a compound of formula (I) that is capable of each of the following: (i) promoting the myelination of an axon, (ii) promoting the differentiation of an OPC into an oligodendrocyte, and (iii) promoting the expression of one or more proteins associated with oligodendrocyte differentiation and/or myelination (e.g., GPR17 and/or MBP).

In some aspects, a PDGFRα inhibitor described herein comprises a compound of formula (II) that is capable of promoting the myelination of an axon. In some aspects, a PDGFRα inhibitor comprises a compound of formula (II) that is capable of (and in some embodiments used for) both promoting the myelination of an axon and promoting the differentiation of an OPC into an oligodendrocyte. In some aspects, a PDGFRα inhibitor comprises a compound of formula (II) that is capable of (and in some embodiments used for) both promoting the myelination of an axon and the expression of one or more proteins associated with oligodendrocyte differentiation and/or myelination (e.g., GPR17 and/or MBP). In some aspects, a PDGFRα inhibitor comprises a compound of formula (II) that is capable of (and in some embodiments used for) each of the following: (i) promoting the myelination of an axon, (ii) promoting the differentiation of an OPC into an oligodendrocyte, and (iii) promoting the expression of one or more proteins associated with oligodendrocyte differentiation and/or myelination (e.g., GPR17 and/or MBP).

In some aspects, a PDGFRα inhibitor described herein comprises a compound of formula (III) that is capable of (and in some embodiments used for) promoting the myelination of an axon. In some aspects, a PDGFRα inhibitor comprises a compound of formula (III) that is capable of (and in some embodiments used for) both promoting the myelination of an axon and promoting the differentiation of an OPC into an oligodendrocyte. In some aspects, a PDGFRα inhibitor comprises a compound of formula (III) that is capable of (and in some embodiments used for) both promoting the myelination of an axon and the expression of one or more proteins associated with oligodendrocyte differentiation and/or myelination (e.g., GPR17 and/or MBP). In some aspects, a PDGFRα inhibitor comprises a compound of formula (III) that is capable of (and in some embodiments used for) each of the following: (i) promoting the myelination of an axon, (ii) promoting the differentiation of an OPC into an oligodendrocyte, and (iii) promoting the expression of one or more proteins associated with oligodendrocyte differentiation and/or myelination (e.g., GPR17 and/or MBP).

In some aspects, a PDGFRα inhibitor described herein comprises a compound of formula (IV) that is capable of (and in some embodiments used for) promoting the myelination of an axon. In some aspects, a PDGFRα inhibitor comprises a compound of formula (IV) that is capable of (and in some embodiments used for) both promoting the myelination of an axon and promoting the differentiation of an OPC into an oligodendrocyte. In some aspects, a PDGFRα inhibitor comprises a compound of formula (IV) that is capable of (and in some embodiments used for) both promoting the myelination of an axon and the expression of one or more proteins associated with oligodendrocyte differentiation and/or myelination (e.g., GPR17 and/or MBP). In some aspects, a PDGFRα inhibitor comprises a compound of formula (IV) that is capable of (and in some embodiments used for) each of the following: (i) promoting the myelination of an axon, (ii) promoting the differentiation of an OPC into an oligodendrocyte, and (iii) promoting the expression of one or more proteins associated with oligodendrocyte differentiation and/or myelination (e.g., GPR17 and/or MBP).

In some aspects, a PDGFRα inhibitor described herein comprises an antagonistic anti-PDGFRα antibody that is capable of (and in some embodiments used for) promoting the myelination of an axon. In some aspects, a PDGFRα inhibitor comprises an antagonistic anti-PDGFRα antibody that is capable of (and in some embodiments used for) both promoting the myelination of an axon and promoting the differentiation of an OPC into an oligodendrocyte. In some aspects, a PDGFRα inhibitor comprises an antagonistic anti-PDGFRα antibody that is capable of (and in some embodiments used for) both promoting the myelination of an axon and the expression of one or more proteins associated with oligodendrocyte differentiation and/or myelination (e.g., GPR17 and/or MBP). In some aspects, a PDGFRα inhibitor comprises an antagonistic anti-PDGFRα antibody that is capable of (and in some embodiments used for) each of the following: (i) promoting the myelination of an axon, (ii) promoting the differentiation of an OPC into an oligodendrocyte, and (iii) promoting the expression of one or more proteins associated with oligodendrocyte differentiation and/or myelination (e.g., GPR17 and/or MBP).

### 4. Promoting the Remyelination of Demyelinated Neuronal Axons

As described herein, in some aspects, a PDGFRα inhibitor useful for the present disclosure can remyelinate a demyelinated neuronal axon. Such remyelination, in some aspects, can help improve one or more functions of a neuron. Non-limiting examples of such functions are provided elsewhere in the present disclosure. In some aspects, when contacted (e.g., in vivo, in vitro, or both) with a PDGFRα inhibitor described herein, the number of neurons with axons that are demyelinated is reduced compared to a reference (e.g., corresponding neurons that are not contacted with the PDGFRα inhibitor). In some aspects, compared to the reference, the amount of neurons with axons that are demyelinated is reduced by at least 5%, at least 10%, at least 20%, at least 30%, at least 40%, at least 50%, at least 60%, at least 70%, at least about 80%, at least 90%, or 100% (for example, at least 50%). The decrease in the number of neurons with axons that are demyelinated can be determined using any suitable methods known in the art or described herein. In some aspects, the decrease in the number of neurons with axons that are demyelinated can be determined by visualizing and/or quantifying the expression of a marker associated with myelinated neurons. For example, in some aspects, the marker associated with myelinated neurons comprises myelin basic protein (MBP), myelin oligodendrocyte glycoprotein (MOG), oligodendrocyte-specific protein/claudin-11, CNPase, or a combination thereof. In some aspects, the decrease in the number of neurons with axons that are demyelinated can be assessed using a cuprizone model, such as that described in Torkildsen et al., Acta Neurol Scand Suppl 188:72-6 (2008) or in Example 243 of the present disclosure.

In some aspects, a PDGFRα inhibitor useful for the present disclosure includes any agent known in the art (e.g., polypeptide, small molecule, antibody, or gene editing tool) that can target PDGFRα and promote the remyelination of demyelinated neuronal axons.

In some aspects, a PDGFRα inhibitor of the present disclosure comprises a compound of formula (I) that is capable of (and in some embodiments used for) promoting the remyelination of demyelinated (e.g., hypomyelinated) neuronal axons. In some aspects, a PDGFRα inhibitor comprises a compound of formula (I) that is capable of (and in some embodiments used for) both promoting the remyelination of demyelinated neuronal axons and promoting the differentiation of an OPC into an oligodendrocyte. In some aspects, a PDGFRα inhibitor comprises a compound of formula (I) that is capable of (and in some embodiments used for) both promoting the remyelination of demyelinated neuronal axons and promoting the expression of one or more proteins associated with oligodendrocyte differentiation and/or myelination (e.g., GPR17 and/or MBP). In some aspects, a PDGFRα inhibitor comprises a compound of formula (I) that is capable of (and in some embodiments used for) both promoting the remyelination of demyelinated neuronal axons and promoting the myelination of an axon (e.g., a hypomyelinated axon). In some aspects, a PDGFRα inhibitor of the present disclosure comprises a compound of formula (I) that is capable of (and in some embodiments used for) each of the following: (i) promoting the remyelination of demyelinated neuronal axons; (ii) promoting the differentiation of an OPC into an oligodendrocyte; and (iii) promoting the expression of one or more proteins associated with oligodendrocyte differentiation and/or myelination (e.g., GPR17 and/or MBP). In some aspects, a PDGFRα inhibitor of the present disclosure comprises a compound of formula (I) that is capable of (and in some embodiments used for) each of the following: (i) promoting the remyelination of demyelinated neuronal axons; (ii) promoting the differentiation of an OPC into an oligodendrocyte; and (iii) promoting the myelination of an axon. In some aspects, a PDGFRα inhibitor of the present disclosure comprises a compound of formula (I) that is capable of (and in some embodiments used for) each of the following: (i) promoting the remyelination of demyelinated neuronal axons; (ii) promoting the expression of one or more proteins associated with oligodendrocyte differentiation and/or myelination (e.g., GPR17 and/or MBP); and (iii) promoting the myelination of an axon.

In some aspects, a PDGFRα inhibitor of the present disclosure comprises a compound of formula (II) that is capable of (and in some embodiments used for) promoting the remyelination of demyelinated neuronal axons. In some aspects, a PDGFRα inhibitor comprises a compound of formula (II) that is capable of (and in some embodiments used for) both promoting the remyelination of demyelinated neuronal axons and promoting the differentiation of an OPC into an oligodendrocyte. In some aspects, a PDGFRα inhibitor comprises a compound of formula (II) that is capable of (and in some embodiments used for) both promoting the remyelination of demyelinated neuronal axons and promoting the expression of one or more proteins associated with oligodendrocyte differentiation and/or myelination (e.g., GPR17 and/or MBP). In some aspects, a PDGFRα inhibitor comprises a compound of formula (II) that is capable of (and in some embodiments used for) both promoting the remyelination of demyelinated neuronal axons and promoting the myelination of an axon (e.g., a hypomyelinated axon). In some aspects, a PDGFRα inhibitor of the present disclosure comprises a compound of formula (II) that is capable of (and in some embodiments used for) each of the following: (i) promoting the remyelination of demyelinated neuronal axons; (ii) promoting the differentiation of an OPC into an oligodendrocyte; and (iii) promoting the expression of one or more proteins associated with oligodendrocyte differentiation and/or myelination (e.g., GPR17 and/or MBP). In some aspects, a PDGFRα inhibitor of the present disclosure comprises a compound of formula (II) that is capable of (and in some embodiments used for) each of the following: (i) promoting the remyelination of demyelinated neuronal axons; (ii) promoting the differentiation of an OPC into an oligodendrocyte; and (iii) promoting the myelination of an axon. In some aspects, a PDGFRα inhibitor of the present disclosure comprises a compound of formula (II) that is capable of (and in some embodiments used for) each of the following: (i) promoting the remyelination of demyelinated neuronal axons; (ii) promoting the expression of one or more proteins associated with oligodendrocyte differentiation and/or myelination (e.g., GPR17 and/or MBP); and (iii) promoting the myelination of an axon.

In some aspects, a PDGFRα inhibitor of the present disclosure comprises a compound of formula (III) that is capable of (and in some embodiments used for) promoting the remyelination of demyelinated neuronal axons. In some aspects, a PDGFRα inhibitor comprises a compound of formula (III) that is capable of (and in some embodiments used for) both promoting the remyelination of demyelinated neuronal axons and promoting the differentiation of an OPC into an oligodendrocyte. In some aspects, a PDGFRα inhibitor comprises a compound of formula (III) that is capable of (and in some embodiments used for) both promoting the remyelination of demyelinated neuronal axons and promoting the expression of one or more proteins associated with oligodendrocyte differentiation and/or myelination (e.g., GPR17 and/or MBP). In some aspects, a PDGFRα inhibitor comprises a compound of formula (III) that is capable of (and in some embodiments used for) both promoting the remyelination of demyelinated neuronal axons and promoting the myelination of an axon (e.g., a hypomyelinated axon). In some aspects, a PDGFRα inhibitor of the present disclosure comprises a compound of formula (III) that is capable of (and in some embodiments used for) each of the following: (i) promoting the remyelination of demyelinated neuronal axons; (ii) promoting the differentiation of an OPC into an oligodendrocyte; and (iii) promoting the expression of one or more proteins associated with oligodendrocyte differentiation and/or myelination (e.g., GPR17 and/or MBP). In some aspects, a PDGFRα inhibitor of the present disclosure comprises a compound of formula (III) that is capable of (and in some embodiments used for) each of the following: (i) promoting the remyelination of demyelinated neuronal axons; (ii) promoting the differentiation of an OPC into an oligodendrocyte; and (iii) promoting the myelination of an axon. In some aspects, a PDGFRα inhibitor of the present disclosure comprises a compound of formula (III) that is capable of (and in some embodiments used for) each of the following: (i) promoting the remyelination of demyelinated neuronal axons; (ii) promoting the expression of one or more proteins associated with oligodendrocyte differentiation and/or myelination (e.g., GPR17 and/or MBP); and (iii) promoting the myelination of an axon.

In some aspects, a PDGFRα inhibitor of the present disclosure comprises a compound of formula (IV) that is capable of (and in some embodiments used for) promoting the remyelination of demyelinated neuronal axons. In some aspects, a PDGFRα inhibitor comprises a compound of formula (IV) that is capable of (and in some embodiments used for) both promoting the remyelination of demyelinated neuronal axons and promoting the differentiation of an OPC into an oligodendrocyte. In some aspects, a PDGFRα inhibitor comprises a compound of formula (IV) that is capable of (and in some embodiments used for) both promoting the remyelination of demyelinated neuronal axons and promoting the expression of one or more proteins associated with oligodendrocyte differentiation and/or myelination (e.g., GPR17 and/or MBP). In some aspects, a PDGFRα inhibitor comprises a compound of formula (IV) that is capable of (and in some embodiments used for) both promoting the remyelination of demyelinated neuronal axons and promoting the myelination of an axon (e.g., a hypomyelinated axon). In some aspects, a PDGFRα inhibitor of the present disclosure comprises a compound of formula (IV) that is capable of (and in some embodiments used for) each of the following: (i) promoting the remyelination of demyelinated neuronal axons; (ii) promoting the differentiation of an OPC into an oligodendrocyte; and (iii) promoting the expression of one or more proteins associated with oligodendrocyte differentiation and/or myelination (e.g., GPR17 and/or MBP). In some aspects, a PDGFRα inhibitor of the present disclosure comprises a compound of formula (IV) that is capable of (and in some embodiments used for) each of the following: (i) promoting the remyelination of demyelinated neuronal axons; (ii) promoting the differentiation of an OPC into an oligodendrocyte; and (iii) promoting the myelination of an axon. In some aspects, a PDGFRα inhibitor of the present disclosure comprises a compound of formula (IV) that is capable of (and in some embodiments used for) each of the following: (i) promoting the remyelination of demyelinated neuronal axons; (ii) promoting the expression of one or more proteins associated with oligodendrocyte differentiation and/or myelination (e.g., GPR17 and/or MBP); and (iii) promoting the myelination of an axon.

In some aspects, a PDGFRα inhibitor of the present disclosure comprises an antagonistic anti-PDGFRα antibody that is capable of (and in some embodiments used for) promoting the remyelination of demyelinated neuronal axons. In some aspects, a PDGFRα inhibitor comprises an antagonistic anti-PDGFRα antibody that is capable of (and in some embodiments used for) both promoting the remyelination of demyelinated neuronal axons and promoting the differentiation of an OPC into an oligodendrocyte. In some aspects, a PDGFRα inhibitor comprises an antagonistic anti-PDGFRα antibody that is capable of (and in some embodiments used for) both promoting the remyelination of demyelinated neuronal axons and promoting the expression of one or more proteins associated with oligodendrocyte differentiation and/or myelination (e.g., GPR17 and/or MBP). In some aspects, a PDGFRα inhibitor comprises an antagonistic anti-PDGFRα antibody that is capable of (and in some embodiments used for) both promoting the remyelination of demyelinated neuronal axons and promoting the myelination of an axon (e.g., a hypomyelinated axon). In some aspects, a PDGFRα inhibitor of the present disclosure comprises an antagonistic anti-PDGFRα antibody that is capable of (and in some embodiments used for) each of the following: (i) promoting the remyelination of demyelinated neuronal axons; (ii) promoting the differentiation of an OPC into an oligodendrocyte; and (iii) promoting the expression of one or more proteins associated with oligodendrocyte differentiation and/or myelination (e.g., GPR17 and/or MBP). In some aspects, a PDGFRα inhibitor of the present disclosure comprises an antagonistic anti-PDGFRα antibody that is capable of (and in some embodiments used for) each of the following: (i) promoting the remyelination of demyelinated neuronal axons; (ii) promoting the differentiation of an OPC into an oligodendrocyte; and (iii) promoting the myelination of an axon. In some aspects, a PDGFRα inhibitor of the present disclosure comprises an antagonistic anti-PDGFRα antibody that is capable of (and in some embodiments used for) each of the following: (i) promoting the remyelination of demyelinated neuronal axons; (ii) promoting the expression of one or more proteins associated with oligodendrocyte differentiation and/or myelination (e.g., GPR17 and/or MBP); and (iii) promoting the myelination of an axon.

### 5. Inhibiting PDGFRα Kinase Activity

PDGFRα inhibitors described herein are capable of inhibiting PDGFRα kinase activity, in some aspects, with much greater potency compared to inhibitors known in the art. As demonstrated herein, in some aspects, compared to certain inhibitors in the art, PDGFRα inhibitors described herein have increased potency, as measured using an assay as described in Example 239. In some aspects, the potency of a PDGFRα inhibitor is determined using an 11-point dose response curve with a maximum concentration of 10,000 nM and 3-fold dilutions to a minimum concentration of 0.169 nM. In some aspects, PDGFRα inhibitors of the present disclosure can inhibit PDGFRα kinase activity with an IC₅₀ of less than 10,000 nM, e.g., as measured using an enzymatic PDGFRα kinase, such as a Promega kinase assay described in Example 239. In some aspects, a PDGFRα inhibitor described herein can inhibit PDGFRα kinase activity with an IC₅₀ of less than 9,000 nM, e.g., as measured using an enzymatic PDGFRα kinase, such as a Promega kinase assay described in Example 239. In some aspects, a PDGFRα inhibitor described herein can inhibit PDGFRα kinase activity with an IC₅₀ of less than 8,000 nM, e.g., as measured using an enzymatic PDGFRα kinase, such as a Promega kinase assay described in Example 239. In some aspects, a PDGFRα inhibitor described herein can inhibit PDGFRα kinase activity with an IC₅₀ of less than 7,000 nM, e.g., as measured using an enzymatic PDGFRα kinase, such as a Promega kinase assay described in Example 239. In some aspects, a PDGFRα inhibitor described herein can inhibit PDGFRα kinase activity with an IC₅₀ of less than 6,000 nM, e.g., as measured using an enzymatic PDGFRα kinase, such as a Promega kinase assay described in Example 239. In some aspects, a PDGFRα inhibitor described herein can inhibit PDGFRα kinase activity with an IC₅₀ of less than 5,000 nM, e.g., as measured using an enzymatic PDGFRα kinase, such as a Promega kinase assay described in Example 239. In some aspects, a PDGFRα inhibitor described herein can inhibit PDGFRα kinase activity with an IC₅₀ of less than 4,000 nM, e.g., as measured using an enzymatic PDGFRα kinase, such as a Promega kinase assay described in Example 239. In some aspects, a PDGFRα inhibitor described herein can inhibit PDGFRα kinase activity with an IC₅₀ of less than 3,000 nM, e.g., as measured using an enzymatic PDGFRα kinase, such as a Promega kinase assay described in Example 239. In some aspects, a PDGFRα inhibitor described herein can inhibit PDGFRα kinase activity with an IC₅₀ of less than 2,000 nM, e.g., as measured using an enzymatic PDGFRα kinase, such as a Promega kinase assay described in Example 239. In some aspects, a PDGFRα inhibitor described herein can inhibit PDGFRα kinase activity with an IC₅₀ of less than 1,000 nM, e.g., as measured using an enzymatic PDGFRα kinase, such as a Promega kinase assay described in Example 239. In some aspects, a PDGFRα inhibitor described herein can inhibit PDGFRα kinase activity with an IC₅₀ of less than 900 nM, e.g., as measured using an enzymatic PDGFRα kinase, such as a Promega kinase assay described in Example 239. In some aspects, a PDGFRα inhibitor described herein can inhibit PDGFRα kinase activity with an IC₅₀ of less than 800 nM, e.g., as measured using an enzymatic PDGFRα kinase, such as a Promega kinase assay described in Example 239. In some aspects, a PDGFRα inhibitor described herein can inhibit PDGFRα kinase activity with an IC₅₀ of less than 700 nM, e.g., as measured using an enzymatic PDGFRα kinase, such as a Promega kinase assay described in Example 239. In some aspects, a PDGFRα inhibitor described herein can inhibit PDGFRα kinase activity with an IC₅₀ of less than 600 nM, e.g., as measured using an enzymatic PDGFRα kinase, such as a Promega kinase assay described in Example 239. In some aspects, a PDGFRα inhibitor described herein can inhibit PDGFRα kinase activity with an IC₅₀ of less than 500 nM, e.g., as measured using an enzymatic PDGFRα kinase, such as a Promega kinase assay described in Example 239. In some aspects, a PDGFRα inhibitor described herein can inhibit PDGFRα kinase activity with an IC₅₀ of less than 400 nM, e.g., as measured using an enzymatic PDGFRα kinase, such as a Promega kinase assay described in Example 239. In some aspects, a PDGFRα inhibitor described herein can inhibit PDGFRα kinase activity with an IC₅₀ of less than 300 nM, e.g., as measured using an enzymatic PDGFRα kinase, such as a Promega kinase assay described in Example 239. In some aspects, a PDGFRα inhibitor described herein can inhibit PDGFRα kinase activity with an IC₅₀ of less than 200 nM., e.g., as measured using an enzymatic PDGFRα kinase, such as a Promega kinase assay described in Example 239. In some aspects, a PDGFRα inhibitor described herein can inhibit PDGFRα kinase activity with an IC₅₀ of less than 100 nM, e.g., as measured using an enzymatic PDGFRα kinase, such as a Promega kinase assay described in Example 239. In some aspects, a PDGFRα inhibitor described herein can inhibit PDGFRα kinase activity with an IC₅₀ of less than 50 nM, e.g., as measured using an enzymatic PDGFRα kinase, such as a Promega kinase assay described in Example 239. In some aspects, a PDGFRα inhibitor described herein can inhibit PDGFRα kinase activity with an IC₅₀ of less than 25 nM, e.g., as measured using an enzymatic PDGFRα kinase, such as a Promega kinase assay described in Example 239. In some aspects, a PDGFRα inhibitor described herein can inhibit PDGFRα kinase activity with an IC₅₀ of less than 10 nM, e.g., as measured using an enzymatic PDGFRα kinase, such as a Promega kinase assay described in Example 239. In some aspects, a PDGFRα inhibitor described herein can inhibit PDGFRα kinase activity with an IC₅₀ of less than 1 nM, e.g., as measured using an enzymatic PDGFRα kinase, such as a Promega kinase assay described in Example 239.

Accordingly, in some aspects, a PDGFRα inhibitor useful for the present disclosure includes any agent known in the art (e.g., polypeptide, small molecule, antibody, or gene editing tool) that can target PDGFRα and inhibit PDGFRα kinase activity with an IC₅₀ of less than 10,000 nM (e.g., less than 9,000 nM, less than 8,000 nM, less than 7,000 nM, less than 6,000 nM, less than 5,000 nM, less than 4,000 nM, less than 3,000 nM, less than 2,000 nM, less than 1,000 nM, less than 900 nM, less than 800 nM, less than 700 nM, less than 600 nM, less than 500 nM, less than 400 nM, less than 300 nM, less than 200 nM, less than 100 nM, less than 50 nM, less than 25 nM, less than 10 nM, or less than 1 nM, for instance, less than 500 nM), e.g., as measured using an enzymatic PDGFRα kinase, such as a Promega kinase assay described in Example 239.

In some aspects, a PDGFRα inhibitor described herein comprises a compound of formula (I) that is capable of (and in some embodiments used for) inhibiting PDGFRα kinase activity with an IC₅₀ of less than 10,000 nM (e.g., less than 9,000 nM, less than 8,000 nM, less than 7,000 nM, less than 6,000 nM, less than 5,000 nM, less than 4,000 nM, less than 3,000 nM, less than 2,000 nM, less than 1,000 nM, less than 900 nM, less than 800 nM, less than 700 nM, less than 600 nM, less than 500 nM, less than 400 nM, less than 300 nM, less than 200 nM, less than 100 nM, less than 50 nM, less than 25 nM, less than 10 nM, or less than 1 nM, for instance, less than 500 nM), e.g., as measured using an enzymatic PDGFRα kinase, such as a Promega kinase assay described in Example 239. In some aspects, a PDGFRα inhibitor comprises a compound of formula (I) that is capable of (and in some embodiments used for) both inhibiting PDGFRα kinase activity with an IC₅₀ of less than 10,000 nM (e.g., less than 1,000 nM, less than 500 nM) and promoting the differentiation of an OPC into an oligodendrocyte. In some aspects, a PDGFRα inhibitor comprises a compound of formula (I) that is capable of (and in some embodiments used for) both inhibiting PDGFRα kinase activity with an IC₅₀ of less than 10,000 nM (e.g., less than 1,000 nM, less than 500 nM) and promoting the expression of one or more proteins associated with oligodendrocyte differentiation and/or myelination (e.g., GPR17 and/or MBP). In some aspects, a PDGFRα inhibitor comprises a compound of formula (I) that is capable of (and in some embodiments used for) both inhibiting PDGFRα kinase activity with an IC₅₀ of less than 10,000 nM (e.g., less than 1,000 nM, less than 500 nM) and promoting the myelination of an axon (e.g., a hypomyelinated axon). In some aspects, a PDGFRα inhibitor comprises a compound of formula (I) that is capable of (and in some embodiments used for) both inhibiting PDGFRα kinase activity with an IC₅₀ of less than 10,000 nM (e.g., less than 1,000 nM, less than 500 nM) and promoting the remyelination of a demyelinated neuronal axon. In some aspects, a PDGFRα inhibitor comprises a compound of formula (I) that is capable of (and in some embodiments used for) each of the following: (i) inhibiting PDGFRα kinase activity with an IC₅₀ of less than 10,000 nM (e.g., less than 1,000 nM, less than 500 nM); (ii) promoting the differentiation of an OPC into an oligodendrocyte; and (iii) promoting the expression of one or more proteins associated with oligodendrocyte differentiation and/or myelination (e.g., GPR17 and/or MBP). In some aspects, a PDGFRα inhibitor comprises a compound of formula (I) that is capable of (and in some embodiments used for) each of the following: (i) inhibiting PDGFRα kinase activity with an IC₅₀ of less than 10,000 nM (e.g., less than 1,000 nM, less than 500 nM); (ii) promoting the differentiation of an OPC into an oligodendrocyte; and (iii) promoting the myelination of an axon. In some aspects, a PDGFRα inhibitor comprises a compound of formula (I) that is capable of (and in some embodiments used for) each of the following: (i) inhibiting PDGFRα kinase activity with an IC₅₀ of less than 10,000 nM (e.g., less than 1,000 nM, less than 500 nM); (ii) promoting the differentiation of an OPC into an oligodendrocyte; and (iii) promoting the remyelination of a demyelinated neuronal axon. In some aspects, a PDGFRα inhibitor comprises a compound of formula (I) that is capable of (and in some embodiments used for) each of the following: (i) inhibiting PDGFRα kinase activity with an IC₅₀ of less than 10,000 nM (e.g., less than 1,000 nM, less than 500 nM); (ii) promoting the expression of one or more proteins associated with oligodendrocyte differentiation and/or myelination (e.g., GPR17 and/or MBP); and (iii) promoting the myelination of an axon. In some aspects, a PDGFRα inhibitor comprises a compound of formula (I) that is capable of (and in some embodiments used for) each of the following: (i) inhibiting PDGFRα kinase activity with an IC₅₀ of less than 10,000 nM (e.g., less than 1,000 nM, less than 500 nM); (ii) promoting the expression of one or more proteins associated with oligodendrocyte differentiation and/or myelination (e.g., GPR17 and/or MBP); and (iii) promoting the remyelination of a demyelinated neuronal axon. In some aspects, a PDGFRα inhibitor comprises a compound of formula (I) that is capable of (and in some embodiments used for) each of the following: (i) inhibiting PDGFRα kinase activity with an IC₅₀ of less than 10,000 nM (e.g., less than 1,000 nM, less than 500 nM); (ii) promoting the myelination of an axon; and (iii) promoting the remyelination of a demyelinated neuronal axon.

In some aspects, a PDGFRα inhibitor described herein comprises a compound of formula (II) that is capable of (and in some embodiments used for) inhibiting PDGFRα kinase activity with an IC₅₀ of less than 10,000 nM (e.g., less than 9,000 nM, less than 8,000 nM, less than 7,000 nM, less than 6,000 nM, less than 5,000 nM, less than 4,000 nM, less than 3,000 nM, less than 2,000 nM, less than 1,000 nM, less than 900 nM, less than 800 nM, less than 700 nM, less than 600 nM, less than 500 nM, less than 400 nM, less than 300 nM, less than 200 nM, less than 100 nM, less than 50 nM, less than 25 nM, less than 10 nM, or less than 1 nM, for instance, less than 500 nM), e.g., as measured using an enzymatic PDGFRα kinase, such as a Promega kinase assay described in Example 239. In some aspects, a PDGFRα inhibitor comprises a compound of formula (II) that is capable of (and in some embodiments used for) both inhibiting PDGFRα kinase activity with an IC₅₀ of less than 10,000 nM (e.g., less than 1,000 nM, less than 500 nM) and promoting the differentiation of an OPC into an oligodendrocyte. In some aspects, a PDGFRα inhibitor comprises a compound of formula (II) that is capable of (and in some embodiments used for) both inhibiting PDGFRα kinase activity with an IC₅₀ of less than 10,000 nM (e.g., less than 1,000 nM, less than 500 nM) and promoting the expression of one or more proteins associated with oligodendrocyte differentiation and/or myelination (e.g., GPR17 and/or MBP). In some aspects, a PDGFRα inhibitor comprises a compound of formula (II) that is capable of (and in some embodiments used for) both inhibiting PDGFRα kinase activity with an IC₅₀ of less than 10,000 nM (e.g., less than 1,000 nM, less than 500 nM) and promoting the myelination of an axon. In some aspects, a PDGFRα inhibitor comprises a compound of formula (II) that is capable of (and in some embodiments used for) both inhibiting PDGFRα kinase activity with an IC₅₀ of less than 10,000 nM (e.g., less than 1,000 nM, less than 500 nM) and promoting the remyelination of a demyelinated neuronal axon. In some aspects, a PDGFRα inhibitor comprises a compound of formula (II) that is capable of (and in some embodiments used for) each of the following: (i) inhibiting PDGFRα kinase activity with an IC₅₀ of less than 10,000 nM (e.g., less than 1,000 nM, less than 500 nM); (ii) promoting the differentiation of an OPC into an oligodendrocyte; and (iii) promoting the expression of one or more proteins associated with oligodendrocyte differentiation and/or myelination (e.g., GPR17 and/or MBP). In some aspects, a PDGFRα inhibitor comprises a compound of formula (II) that is capable of (and in some embodiments used for) each of the following: (i) inhibiting PDGFRα kinase activity with an IC₅₀ of less than 10,000 nM (e.g., less than 1,000 nM, less than 500 nM); (ii) promoting the differentiation of an OPC into an oligodendrocyte; and (iii) promoting the myelination of an axon. In some aspects, a PDGFRα inhibitor comprises a compound of formula (II) that is capable of (and in some embodiments used for) each of the following: (i) inhibiting PDGFRα kinase activity with an IC₅₀ of less than 10,000 nM (e.g., less than 1,000 nM, less than 500 nM); (ii) promoting the differentiation of an OPC into an oligodendrocyte; and (iii) promoting the remyelination of a demyelinated neuronal axon. In some aspects, a PDGFRα inhibitor comprises a compound of formula (II) that is capable of (and in some embodiments used for) each of the following: (i) inhibiting PDGFRα kinase activity with an IC₅₀ of less than 10,000 nM (e.g., less than 1,000 nM, less than 500 nM); (ii) promoting the expression of one or more proteins associated with oligodendrocyte differentiation and/or myelination (e.g., GPR17 and/or MBP); and (iii) promoting the myelination of an axon. In some aspects, a PDGFRα inhibitor comprises a compound of formula (II) that is capable of (and in some embodiments used for) each of the following: (i) inhibiting PDGFRα kinase activity with an IC₅₀ of less than 10,000 nM (e.g., less than 1,000 nM, less than 500 nM); (ii) promoting the expression of one or more proteins associated with oligodendrocyte differentiation and/or myelination (e.g., GPR17 and/or MBP); and (iii) promoting the remyelination of a demyelinated neuronal axon. In some aspects, a PDGFRα inhibitor comprises a compound of formula (II) that is capable of (and in some embodiments used for) each of the following: (i) inhibiting PDGFRα kinase activity with an IC₅₀ of less than 10,000 nM (e.g., less than 1,000 nM, less than 500 nM); (ii) promoting the myelination of an axon; and (iii) promoting the remyelination of a demyelinated neuronal axon.

In some aspects, a PDGFRα inhibitor described herein comprises a compound of formula (III) that is capable of (and in some embodiments used for) inhibiting PDGFRα kinase activity with an IC₅₀ of less than 10,000 nM (e.g., less than 9,000 nM, less than 8,000 nM, less than 7,000 nM, less than 6,000 nM, less than 5,000 nM, less than 4,000 nM, less than 3,000 nM, less than 2,000 nM, less than 1,000 nM, less than 900 nM, less than 800 nM, less than 700 nM, less than 600 nM, less than 500 nM, less than 400 nM, less than 300 nM, less than 200 nM, less than 100 nM, less than 50 nM, less than 25 nM, less than 10 nM, or less than 1 nM, for instance, less than 500 nM), e.g., as measured using an enzymatic PDGFRα kinase, such as a Promega kinase assay described in Example 239. In some aspects, a PDGFRα inhibitor comprises a compound of formula (III) that is capable of (and in some embodiments used for) both inhibiting PDGFRα kinase activity with an IC₅₀ of less than 10,000 nM (e.g., less than 1,000 nM, less than 500 nM) and promoting the differentiation of an OPC into an oligodendrocyte. In some aspects, a PDGFRα inhibitor comprises a compound of formula (III) that is capable of (and in some embodiments used for) both inhibiting PDGFRα kinase activity with an IC₅₀ of less than 10,000 nM (e.g., less than 1,000 nM, less than 500 nM) and promoting the expression of one or more proteins associated with oligodendrocyte differentiation and/or myelination (e.g., GPR17 and/or MBP). In some aspects, a PDGFRα inhibitor comprises a compound of formula (III) that is capable of (and in some embodiments used for) both inhibiting PDGFRα kinase activity with an IC₅₀ of less than 10,000 nM (e.g., less than 1,000 nM, less than 500 nM) and promoting the myelination of an axon. In some aspects, a PDGFRα inhibitor comprises a compound of formula (III) that is capable of (and in some embodiments used for) both inhibiting PDGFRα kinase activity with an IC₅₀ of less than 10,000 nM (e.g., less than 1,000 nM, less than 500 nM) and promoting the remyelination of a demyelinated neuronal axon. In some aspects, a PDGFRα inhibitor comprises a compound of formula (III) that is capable of (and in some embodiments used for) each of the following: (i) inhibiting PDGFRα kinase activity with an IC₅₀ of less than 10,000 nM (e.g., less than 1,000 nM, less than 500 nM); (ii) promoting the differentiation of an OPC into an oligodendrocyte; and (iii) promoting the expression of one or more proteins associated with oligodendrocyte differentiation and/or myelination (e.g., GPR17 and/or MBP). In some aspects, a PDGFRα inhibitor comprises a compound of formula (III) that is capable of (and in some embodiments used for) each of the following: (i) inhibiting PDGFRα kinase activity with an IC₅₀ of less than 10,000 nM (e.g., less than 1,000 nM, less than 500 nM); (ii) promoting the differentiation of an OPC into an oligodendrocyte; and (iii) promoting the myelination of an axon. In some aspects, a PDGFRα inhibitor comprises a compound of formula (III) that is capable of (and in some embodiments used for) each of the following: (i) inhibiting PDGFRα kinase activity with an IC₅₀ of less than 10,000 nM (e.g., less than 1,000 nM, less than 500 nM); (ii) promoting the differentiation of an OPC into an oligodendrocyte; and (iii) promoting the remyelination of a demyelinated neuronal axon. In some aspects, a PDGFRα inhibitor comprises a compound of formula (III) that is capable of (and in some embodiments used for) each of the following: (i) inhibiting PDGFRα kinase activity with an IC₅₀ of less than 10,000 nM (e.g., less than 1,000 nM, less than 500 nM); (ii) promoting the expression of one or more proteins associated with oligodendrocyte differentiation and/or myelination (e.g., GPR17 and/or MBP); and (iii) promoting the myelination of an axon. In some aspects, a PDGFRα inhibitor comprises a compound of formula (III) that is capable of (and in some embodiments used for) each of the following: (i) inhibiting PDGFRα kinase activity with an IC₅₀ of less than 10,000 nM (e.g., less than 1,000 nM, less than 500 nM); (ii) promoting the expression of one or more proteins associated with oligodendrocyte differentiation and/or myelination (e.g., GPR17 and/or MBP); and (iii) promoting the remyelination of a demyelinated neuronal axon. In some aspects, a PDGFRα inhibitor comprises a compound of formula (III) that is capable of (and in some embodiments used for) each of the following: (i) inhibiting PDGFRα kinase activity with an IC₅₀ of less than 10,000 nM (e.g., less than 1,000 nM, less than 500 nM); (ii) promoting the myelination of an axon; and (iii) promoting the remyelination of a demyelinated neuronal axon.

In some aspects, a PDGFRα inhibitor described herein comprises a compound of formula (IV) that is capable of (and in some embodiments used for) inhibiting PDGFRα kinase activity with an IC₅₀ of less than 10,000 nM (e.g., less than 9,000 nM, less than 8,000 nM, less than 7,000 nM, less than 6,000 nM, less than 5,000 nM, less than 4,000 nM, less than 3,000 nM, less than 2,000 nM, less than 1,000 nM, less than 900 nM, less than 800 nM, less than 700 nM, less than 600 nM, less than 500 nM, less than 400 nM, less than 300 nM, less than 200 nM, less than 100 nM, less than 50 nM, less than 25 nM, less than 10 nM, or less than 1 nM, for instance, less than 500 nM), e.g., as measured using an enzymatic PDGFRα kinase, such as a Promega kinase assay described in Example 239. In some aspects, a PDGFRα inhibitor comprises a compound of formula (IV) that is capable of (and in some embodiments used for) both inhibiting PDGFRα kinase activity with an IC₅₀ of less than 10,000 nM (e.g., less than 1,000 nM, less than 500 nM) and promoting the differentiation of an OPC into an oligodendrocyte. In some aspects, a PDGFRα inhibitor comprises a compound of formula (IV) that is capable of (and in some embodiments used for) both inhibiting PDGFRα kinase activity with an IC₅₀ of less than 10,000 nM (e.g., less than 1,000 nM, less than 500 nM) and promoting the expression of one or more proteins associated with oligodendrocyte differentiation and/or myelination (e.g., GPR17 and/or MBP). In some aspects, a PDGFRα inhibitor comprises a compound of formula (IV) that is capable of (and in some embodiments used for) both inhibiting PDGFRα kinase activity with an IC₅₀ of less than 10,000 nM (e.g., less than 1,000 nM, less than 500 nM) and promoting the myelination of an axon. In some aspects, a PDGFRα inhibitor comprises a compound of formula (IV) that is capable of (and in some embodiments used for) both inhibiting PDGFRα kinase activity with an IC₅₀ of less than 10,000 nM (e.g., less than 1,000 nM, less than 500 nM) and promoting the remyelination of a demyelinated neuronal axon. In some aspects, a PDGFRα inhibitor comprises a compound of formula (IV) that is capable of (and in some embodiments used for) each of the following: (i) inhibiting PDGFRα kinase activity with an IC₅₀ of less than 10,000 nM (e.g., less than 1,000 nM, less than 500 nM); (ii) promoting the differentiation of an OPC into an oligodendrocyte; and (iii) promoting the expression of one or more proteins associated with oligodendrocyte differentiation and/or myelination (e.g., GPR17 and/or MBP). In some aspects, a PDGFRα inhibitor comprises a compound of formula (IV) that is capable of (and in some embodiments used for) each of the following: (i) inhibiting PDGFRα kinase activity with an IC₅₀ of less than 10,000 nM (e.g., less than 1,000 nM, less than 500 nM); (ii) promoting the differentiation of an OPC into an oligodendrocyte; and (iii) promoting the myelination of an axon. In some aspects, a PDGFRα inhibitor comprises a compound of formula (IV) that is capable of (and in some embodiments used for) each of the following: (i) inhibiting PDGFRα kinase activity with an IC₅₀ of less than 10,000 nM (e.g., less than 1,000 nM, less than 500 nM); (ii) promoting the differentiation of an OPC into an oligodendrocyte; and (iii) promoting the remyelination of a demyelinated neuronal axon. In some aspects, a PDGFRα inhibitor comprises a compound of formula (IV) that is capable of (and in some embodiments used for) each of the following: (i) inhibiting PDGFRα kinase activity with an IC₅₀ of less than 10,000 nM (e.g., less than 1,000 nM, less than 500 nM); (ii) promoting the expression of one or more proteins associated with oligodendrocyte differentiation and/or myelination (e.g., GPR17 and/or MBP); and (iii) promoting the myelination of an axon. In some aspects, a PDGFRα inhibitor comprises a compound of formula (IV) that is capable of (and in some embodiments used for) each of the following: (i) inhibiting PDGFRα kinase activity with an IC₅₀ of less than 10,000 nM (e.g., less than 1,000 nM, less than 500 nM); (ii) promoting the expression of one or more proteins associated with oligodendrocyte differentiation and/or myelination (e.g., GPR17 and/or MBP); and (iii) promoting the remyelination of a demyelinated neuronal axon. In some aspects, a PDGFRα inhibitor comprises a compound of formula (IV) that is capable of (and in some embodiments used for) each of the following: (i) inhibiting PDGFRα kinase activity with an IC₅₀ of less than 10,000 nM (e.g., less than 1,000 nM, less than 500 nM); (ii) promoting the myelination of an axon; and (iii) promoting the remyelination of a demyelinated neuronal axon.

In some aspects, a PDGFRα inhibitor described herein comprises an antagonistic anti-PDGFRα antibody that is capable of (and in some embodiments used for) inhibiting PDGFRα kinase activity with an IC₅₀ of less than 10,000 nM (e.g., less than 9,000 nM, less than 8,000 nM, less than 7,000 nM, less than 6,000 nM, less than 5,000 nM, less than 4,000 nM, less than 3,000 nM, less than 2,000 nM, less than 1,000 nM, less than 900 nM, less than 800 nM, less than 700 nM, less than 600 nM, less than 500 nM, less than 400 nM, less than 300 nM, less than 200 nM, less than 100 nM, less than 50 nM, less than 25 nM, less than 10 nM, or less than 1 nM, for instance, less than 500 nM), e.g., as measured using an enzymatic PDGFRα kinase, such as a Promega kinase assay described in Example 239. In some aspects, a PDGFRα inhibitor comprises an antagonistic anti-PDGFRα antibody that is capable of (and in some embodiments used for) both inhibiting PDGFRα kinase activity with an IC₅₀ of less than 10,000 nM (e.g., less than 1,000 nM, less than 500 nM) and promoting the differentiation of an OPC into an oligodendrocyte. In some aspects, a PDGFRα inhibitor comprises an antagonistic anti-PDGFRα antibody that is capable of (and in some embodiments used for) both inhibiting PDGFRα kinase activity with an IC₅₀ of less than 10,000 nM (e.g., less than 1,000 nM, less than 500 nM) and promoting the expression of one or more proteins associated with oligodendrocyte differentiation and/or myelination (e.g., GPR17 and/or MBP). In some aspects, a PDGFRα inhibitor comprises an antagonistic anti-PDGFRα antibody that is capable of (and in some embodiments used for) both inhibiting PDGFRα kinase activity with an IC₅₀ of less than 10,000 nM (e.g., less than 1,000 nM, less than 500 nM) and promoting the myelination of an axon. In some aspects, a PDGFRα inhibitor comprises an antagonistic anti-PDGFRα antibody that is capable of (and in some embodiments used for) both inhibiting PDGFRα kinase activity with an IC₅₀ of less than 10,000 nM (e.g., less than 1,000 nM, less than 500 nM) and promoting the remyelination of a demyelinated neuronal axon. In some aspects, a PDGFRα inhibitor comprises an antagonistic anti-PDGFRα antibody that is capable of (and in some embodiments used for) each of the following: (i) inhibiting PDGFRα kinase activity with an IC₅₀ of less than 10,000 nM (e.g., less than 1,000 nM, less than 500 nM); (ii) promoting the differentiation of an OPC into an oligodendrocyte; and (iii) promoting the expression of one or more proteins associated with oligodendrocyte differentiation and/or myelination (e.g., GPR17 and/or MBP). In some aspects, a PDGFRα inhibitor comprises an antagonistic anti-PDGFRα antibody that is capable of (and in some embodiments used for) each of the following: (i) inhibiting PDGFRα kinase activity with an IC₅₀ of less than 10,000 nM (e.g., less than 1,000 nM, less than 500 nM); (ii) promoting the differentiation of an OPC into an oligodendrocyte; and (iii) promoting the myelination of an axon. In some aspects, a PDGFRα inhibitor comprises an antagonistic anti-PDGFRα antibody that is capable of (and in some embodiments used for) each of the following: (i) inhibiting PDGFRα kinase activity with an IC₅₀ of less than 10,000 nM (e.g., less than 1,000 nM, less than 500 nM); (ii) promoting the differentiation of an OPC into an oligodendrocyte; and (iii) promoting the remyelination of a demyelinated neuronal axon. In some aspects, a PDGFRα inhibitor comprises an antagonistic anti-PDGFRα antibody that is capable of (and in some embodiments used for) each of the following: (i) inhibiting PDGFRα kinase activity with an IC₅₀ of less than 10,000 nM; (ii) promoting the expression of one or more proteins associated with oligodendrocyte differentiation and/or myelination (e.g., GPR17 and/or MBP); and (iii) promoting the myelination of an axon. In some aspects, a PDGFRα inhibitor comprises an antagonistic anti-PDGFRα antibody that is capable of each of the following: (i) inhibiting PDGFRα kinase activity with an IC₅₀ of less than 1,000 nM, less than 500 nM); (ii) promoting the expression of one or more proteins associated with oligodendrocyte differentiation and/or myelination (e.g., GPR17 and/or MBP); and (iii) promoting the myelination of an axon. In some aspects, a PDGFRα inhibitor comprises an antagonistic anti-PDGFRα antibody that is capable of each of the following: (i) inhibiting PDGFRα kinase activity with an IC₅₀ of less than 1000 nM; (ii) promoting the myelination of an axon; and (iii) promoting the remyelination of a demyelinated neuronal axon.

Generally, compounds with high potency can exhibit a given therapeutic effect at a reduced dosage compared to compounds with lower potency. Accordingly, compared to other inhibitors available in the art, the PDGFRα inhibitors useful for the present disclosure can inhibit PDGFRα activity (e.g., kinase activity) to a greater extent at a fixed dosage. In some aspects, at a concentration of 1 uM, a PDGFRα inhibitor described herein (e.g., compounds of formula I, II, III, or IV) can inhibit at least 40%, at least 45%, at least 50%, at least 55%, at least 60%, at least 65%, at least 70%, at least 75%, at least 80%, at least 85%, at least 90%, at least 95%, or 100% of the PDGFRα activity of a nervous system cell (e.g., OPC). In some aspects, at a concentration of 1 uM, a PDGFRα inhibitor described herein can inhibit at least 55% of the PDGFRα activity of a nervous system cell (e.g., OPC).

As described herein, in some aspects, the IC₅₀ of the PDGFRα inhibitor can be determined using an enzymatic PDGFRα kinase assay (e.g., Promega kinase assay described in Example 239). In some aspects, the enzymatic PDGFRα kinase assay comprises 20 ng of purified PDGFRα protein, 150 uM of ATP and 1 μg of substrate, Poly (Glu4Tyr1) in a volume of 15 ul.

### 6. Penetrating the CNS at Sufficient Levels to Support CNS Activity

As further described elsewhere in the present disclosure, the CNS is protected by a blood-brain barrier (BBB) that prevents material in the circulating blood from non-selectively crossing into the extracellular fluid of the CNS where nervous system cells (e.g., neurons) reside. The ability to efficiently cross such a barrier and penetrate the CNS can be advantageous in constructing a PDGFRα inhibitor to myelinate an axon (e.g., a hypomyelinated axon) and/or remyelinate demyelinated neuronal axons.

In some aspects, a PDGFRα inhibitor described herein is capable of (and in some embodiments used for) penetrating the CNS at a sufficient level, such that the inhibitor can exert its therapeutic activities (e.g., promote the myelination of an axon (e.g., a hypomyelinated axon) and/or promote the remyelination of demyelinated neuronal axons) within the CNS. In some aspects, after systemic administration, the ratio of the amount of PDGFRα inhibitors present in the brain compared to the blood/plasma (i.e., “brain to plasma ratio”) is greater than 0.1 (e.g., greater than 0.2, greater than 0.3, greater than 0.4, greater than 0.5, greater than 0.6, greater than 0.7, greater than 0.8, greater than 0.9, greater than 1.0, greater than 1.1, greater than 1.2, greater than 1.3, greater than 1.4, greater than 1.5, greater than 1.6, greater than 1.7, greater than 1.8, greater than 1.9, or greater than 2.0). In some aspects, the brain to plasma ratio is between 0.1 and 2.0. In some aspects, the brain to plasma ratio is between 0.2 and 2.0. In some aspects, the brain to plasma ratio is between 0.3 and 2.0. In some aspects, the brain to plasma ratio is between 0.4 and 2.0. In some aspects, the brain to plasma ratio is between 0.5 and 2.0. In some aspects, the brain to plasma ratio is between 0.5 and 2.0. In some aspects, the brain to plasma ratio is between 0.6 and 2.0. In some aspects, the brain to plasma ratio is between 0.7 and 2.0. In some aspects, the brain to plasma ratio is between 0.8 and 2.0. In some aspects, the brain to plasma ratio is between 0.9 and 2.0. In some aspects, the brain to plasma ratio is between 1.0 and 2.0. In some aspects, the brain to plasma ratio is between 1.1 and 2.0. In some aspects, the brain to plasma ratio is between 1.2 and 2.0. In some aspects, the brain to plasma ratio is between 1.3 and 2.0. In some aspects, the brain to plasma ratio is between 1.4 and 2.0. In some aspects, the brain to plasma ratio is between 1.5 and 2.0. In some aspects, the brain to plasma ratio is between 1.6 and 2.0. In some aspects, the brain to plasma ratio is between 1.7 and 2.0. In some aspects, the brain to plasma ratio is between 1.8 and 2.0. In some aspects, the brain to plasma ratio is between 1.9 and 2.0. In some aspects, the brain to plasma ratio is between 0.1 and 1.5. In some aspects, the brain to plasma ratio is between 0.2 and 1.5. In some aspects, the brain to plasma ratio is between 0.3 and 1.5. In some aspects, the brain to plasma ratio is between 0.4 and 1.5. In some aspects, the brain to plasma ratio is between 0.5 and 1.5. In some aspects, the brain to plasma ratio is between 0.6 and 1.5. In some aspects, the brain to plasma ratio is between 0.7 and 1.5. In some aspects, the brain to plasma ratio is between 0.8 and 1.5. In some aspects, the brain to plasma ratio is between 0.9 and 1.5. In some aspects, the brain to plasma ratio is between 1.0 and 1.5. In some aspects, the brain to plasma ration is between 1.1 and 1.5. In some aspects, the brain to plasma ratio is between 1.2 and 1.5. In some aspects, the brain to plasma ratio is between 1.3 and 1.5. In some aspects, the brain to plasma ratio is between 1.4 and 1.5. In some aspects, the brain to plasma ratio is between 0.1 and 1.0. In some aspects, the brain to plasma ratio is between 0.2 and 1.0. In some aspects, the brain to plasma ratio is between 0.3 and 1.0. In some aspects, the brain to plasma ratio is between 0.5 and 1.0. In some aspects, the brain to plasma ratio is between 0.6 and 1.0. In some aspects, the brain to plasma ratio is between 0.7 and 1.0. In some aspects, the brain to plasma ratio is between 0.8 and 1.0. In some aspects, the brain to plasma ratio is between 0.9 and 1.0. Not to be bound by any one theory, in some aspects, an increased amount of PDGFRα inhibitor that can penetrate into the CNS can allow for greater myelination of an axon (e.g., hypomyelinated axons), the remyelination of demyelinated neuronal axons, or both.

In some aspects, a PDGFRα inhibitor useful for the present disclosure includes any agent known in the art (e.g., polypeptide, small molecule, antibody, or gene editing tool) that can target PDGFRα and achieve a brain to plasma ratio of greater than 0.1 when systemically administered to a subject.

In some aspects, a PDGFRα inhibitor that can be used with the present disclosure comprises a compound of formula (I) that is capable of (and in some embodiments used for) achieving a brain to plasma ratio of greater than 0.1 when systemically administered to a subject. In some aspects, the PDGFRα inhibitor comprises a compound of formula (I) that is capable of (and in some embodiments used for) both achieving a brain to plasma ratio of greater than 0.1 when systemically administered to a subject and promoting the differentiation of an OPC into an oligodendrocyte. In some aspects, the PDGFRα inhibitor comprises a compound of formula (I) that is capable of (and in some embodiments used for) both achieving a brain to plasma ratio of greater than 0.1 when systemically administered to a subject and promoting the expression of one or more proteins associated with oligodendrocyte differentiation and/or myelination (e.g., GPR17 and/or MBP). In some aspects, the PDGFRα inhibitor comprises a compound of formula (I) that is capable of (and in some embodiments used for) both achieving a brain to plasma ratio of greater than 0.1 when systemically administered to a subject and promoting the myelination of an axon (e.g., a hypomyelinated axon). In some aspects, the PDGFRα inhibitor comprises a compound of formula (I) that is capable of (and in some embodiments used for) both achieving a brain to plasma ratio of greater than 0.1 when systemically administered to a subject and promoting the remyelination of a demyelinated neuronal axon. In some aspects, the PDGFRα inhibitor comprises a compound of formula (I) that is capable of (and in some embodiments used for) both achieving a brain to plasma ratio of greater than 0.1 when systemically administered to a subject and inhibiting PDGFRα kinase activity with an IC₅₀ of less than 10,000 nM (e.g., less than 1,000 nM, less than 500 nM).

In some aspects, the PDGFRα inhibitor comprises a compound of formula (I) that is capable of (and in some embodiments used for) each of the following: (i) achieving a brain to plasma ratio of greater than 0.1 when systemically administered to a subject; (ii) promoting the differentiation of an OPC into an oligodendrocyte; and (iii) promoting the expression of one or more proteins associated with oligodendrocyte differentiation and/or myelination (e.g., GPR17 and/or MBP). In some aspects, the PDGFRα inhibitor comprises a compound of formula (I) that is capable of (and in some embodiments used for) each of the following: (i) achieving a brain to plasma ratio of greater than 0.1 when systemically administered to a subject; (ii) promoting the differentiation of an OPC into an oligodendrocyte; and (iii) promoting the myelination of an axon. In some aspects, the PDGFRα inhibitor comprises a compound of formula (I) that is capable of (and in some embodiments used for) each of the following: (i) achieving a brain to plasma ratio of greater than 0.1 when systemically administered to a subject; (ii) promoting the differentiation of an OPC into an oligodendrocyte; and (iii) promoting the remyelination of a demyelinated neuronal axon. In some aspects, the PDGFRα inhibitor comprises a compound of formula (I) that is capable of (and in some embodiments used for) each of the following: (i) achieving a brain to plasma ratio of greater than 0.1 when systemically administered to a subject; (ii) promoting the differentiation of an OPC into an oligodendrocyte; and (iii) inhibiting PDGFRα kinase activity with an IC₅₀ of less than 10,000 nM (e.g., less than 1,000 nM, less than 500 nM). In some aspects, the PDGFRα inhibitor comprises a compound of formula (I) that is capable of (and in some embodiments used for) each of the following: (i) achieving a brain to plasma ratio of greater than 0.1 when systemically administered to a subject; (ii) promoting the expression of one or more proteins associated with oligodendrocyte differentiation and/or myelination (e.g., GPR17 and/or MBP); and (iii) promoting the myelination of an axon. In some aspects, the PDGFRα inhibitor comprises a compound of formula (I) that is capable of (and in some embodiments used for) each of the following: (i) achieving a brain to plasma ratio of greater than 0.1 when systemically administered to a subject; (ii) promoting the expression of one or more proteins associated with oligodendrocyte differentiation and/or myelination (e.g., GPR17 and/or MBP); and (iii) promoting the remyelination of a demyelinated neuronal axon. In some aspects, the PDGFRα inhibitor comprises a compound of formula (I) that is capable of (and in some embodiments used for) each of the following: (i) achieving a brain to plasma ratio of greater than 0.1 when systemically administered to a subject; (ii) promoting the expression of one or more proteins associated with oligodendrocyte differentiation and/or myelination (e.g., GPR17 and/or MBP); and (iii) inhibiting PDGFRα kinase activity with an IC₅₀ of less than 10,000 nM (e.g., less than 1,000 nM, less than 500 nM). In some aspects, the PDGFRα inhibitor comprises a compound of formula (I) that is capable of (and in some embodiments used for) each of the following: (i) achieving a brain to plasma ratio of greater than 0.1 when systemically administered to a subject; (ii) promoting the myelination of an axon; and (iii) promoting the remyelination of a demyelinated neuronal axon. In some aspects, the PDGFRα inhibitor comprises a compound of formula (I) that is capable of (and in some embodiments used for) each of the following: (i) achieving a brain to plasma ratio of greater than 0.1 when systemically administered to a subject; (ii) promoting the myelination of an axon; and (iii) inhibiting PDGFRα kinase activity with an IC₅₀ of less than 10,000 nM (e.g., less than 1,000 nM, less than 500 nM). In some aspects, the PDGFRα inhibitor comprises a compound of formula (I) that is capable of (and in some embodiments used for) each of the following: (i) achieving a brain to plasma ratio of greater than 0.1 when systemically administered to a subject; (ii) promoting the remyelination of a demyelinated neuronal axon; and (iii) inhibiting PDGFRα kinase activity with an IC₅₀ of less than 10,000 nM (e.g., less than 1,000 nM, less than 500 nM).

In some aspects, the PDGFRα inhibitor comprises a compound of formula (I) that is capable of (and in some embodiments used for) each of the following: (i) achieving a brain to plasma ratio of greater than 0.1 when systemically administered to a subject; (ii) promoting the differentiation of an OPC into an oligodendrocyte; (iii) promoting the expression of one or more proteins associated with oligodendrocyte differentiation and/or myelination (e.g., GPR17 and/or MBP); and (iv) promoting the myelination of an axon. In some aspects, the PDGFRα inhibitor comprises a compound of formula (I) that is capable of (and in some embodiments used for) each of the following: (i) achieving a brain to plasma ratio of greater than 0.1 when systemically administered to a subject; (ii) promoting the differentiation of an OPC into an oligodendrocyte; (iii) promoting the expression of one or more proteins associated with oligodendrocyte differentiation and/or myelination (e.g., GPR17 and/or MBP); and (iv) promoting the remyelination of a demyelinated neuronal axon. In some aspects, the PDGFRα inhibitor comprises a compound of formula (I) that is capable of (and in some embodiments used for) each of the following: (i) achieving a brain to plasma ratio of greater than 0.1 when systemically administered to a subject; (ii) promoting the differentiation of an OPC into an oligodendrocyte; (iii) promoting the expression of one or more proteins associated with oligodendrocyte differentiation and/or myelination (e.g., GPR17 and/or MBP); and (iv) inhibiting PDGFRα kinase activity with an IC₅₀ of less than 10,000 nM (e.g., less than 1,000 nM, less than 500 nM). In some aspects, the PDGFRα inhibitor comprises a compound of formula (I) that is capable of (and in some embodiments used for) each of the following: (i) achieving a brain to plasma ratio of greater than 0.1 when systemically administered to a subject; (ii) promoting the expression of one or more proteins associated with oligodendrocyte differentiation and/or myelination (e.g., GPR17 and/or MBP); (iii) promoting the myelination of an axon; and (iv) promoting the remyelination of a demyelinated neuronal axon. In some aspects, the PDGFRα inhibitor comprises a compound of formula (I) that is capable of (and in some embodiments used for) each of the following: (i) achieving a brain to plasma ratio of greater than 0.1 when systemically administered to a subject; (ii) promoting the expression of one or more proteins associated with oligodendrocyte differentiation and/or myelination (e.g., GPR17 and/or MBP); (iii) promoting the myelination of an axon; and (iv) inhibiting PDGFRα kinase activity with an IC₅₀ of less than 10,000 nM (e.g., less than 1,000 nM, less than 500 nM). In some aspects, the PDGFRα inhibitor comprises a compound of formula (I) that is capable of (and in some embodiments used for) each of the following: (i) achieving a brain to plasma ratio of greater than 0.1 when systemically administered to a subject; (ii) promoting the myelination of an axon; (iii) promoting the remyelination of a demyelinated neuronal axon; and (iv) inhibiting PDGFRα kinase activity with an IC₅₀ of less than 10,000 nM (e.g., less than 1,000 nM, less than 500 nM).

In some aspects, the PDGFRα inhibitor comprises a compound of formula (I) that is capable of (and in some embodiments used for) each of the following: (i) achieving a brain to plasma ratio of greater than 0.1 when systemically administered to a subject; (ii) promoting the differentiation of an OPC into an oligodendrocyte; (iii) promoting the expression of one or more proteins associated with oligodendrocyte differentiation and/or myelination (e.g., GPR17 and/or MBP); (iv) promoting the myelination of an axon; and (v) promoting the remyelination of a demyelinated neuronal axon. In some aspects, the PDGFRα inhibitor comprises a compound of formula (I) that is capable of (and in some embodiments used for) each of the following: (i) achieving a brain to plasma ratio of greater than 0.1 when systemically administered to a subject; (ii) promoting the differentiation of an OPC into an oligodendrocyte; (iii) promoting the expression of one or more proteins associated with oligodendrocyte differentiation and/or myelination (e.g., GPR17 and/or MBP); (iv) promoting the myelination of an axon; and (v) inhibiting PDGFRα kinase activity with an IC₅₀ of less than 10,000 nM (e.g., less than 1,000 nM, less than 500 nM). In some aspects, the PDGFRα inhibitor comprises a compound of formula (I) that is capable of (and in some embodiments used for) each of the following: (i) achieving a brain to plasma ratio of greater than 0.1 when systemically administered to a subject; (ii) promoting the expression of one or more proteins associated with oligodendrocyte differentiation and/or myelination (e.g., GPR17 and/or MBP); (iii) promoting the myelination of an axon; (iv) promoting the remyelination of a demyelinated neuronal axon; and (v) inhibiting PDGFRα kinase activity with an IC₅₀ of less than 10,000 nM (e.g., less than 1,000 nM, less than 500 nM).

In some aspects, the PDGFRα inhibitor comprises a compound of formula (I) that is capable of (and in some embodiments used for) each of the following: (i) achieving a brain to plasma ratio of greater than 0.1 when systemically administered to a subject; (ii) promoting the differentiation of an OPC into an oligodendrocyte; (iii) promoting the expression of one or more proteins associated with oligodendrocyte differentiation and/or myelination (e.g., GPR17 and/or MBP); (iv) promoting the myelination of an axon; (v) promoting the remyelination of a demyelinated neuronal axon; and (vi) inhibiting PDGFRα kinase activity with an IC₅₀ of less than 10,000 nM (e.g., less than 1,000 nM, less than 500 nM).

In some aspects, a PDGFRα inhibitor that can be used with the present disclosure comprises a compound of formula (II) that is capable of (and in some embodiments used for) achieving a brain to plasma ratio of greater than 0.1 when systemically administered to a subject. In some aspects, the PDGFRα inhibitor comprises a compound of formula (II) that is capable of (and in some embodiments used for) both achieving a brain to plasma ratio of greater than 0.1 when systemically administered to a subject and promoting the differentiation of an OPC into an oligodendrocyte. In some aspects, the PDGFRα inhibitor comprises a compound of formula (II) that is capable of (and in some embodiments used for) both achieving a brain to plasma ratio of greater than 0.1 when systemically administered to a subject and promoting the expression of one or more proteins associated with oligodendrocyte differentiation and/or myelination (e.g., GPR17 and/or MBP). In some aspects, the PDGFRα inhibitor comprises a compound of formula (II) that is capable of (and in some embodiments used for) both achieving a brain to plasma ratio of greater than 0.1 when systemically administered to a subject and promoting the myelination of an axon. In some aspects, the PDGFRα inhibitor comprises a compound of formula (II) that is capable of (and in some embodiments used for) both achieving a brain to plasma ratio of greater than 0.1 when systemically administered to a subject and promoting the remyelination of a demyelinated neuronal axon. In some aspects, the PDGFRα inhibitor comprises a compound of formula (II) that is capable of (and in some embodiments used for) both achieving a brain to plasma ratio of greater than 0.1 when systemically administered to a subject and inhibiting PDGFRα kinase activity with an IC₅₀ of less than 10,000 nM (e.g., less than 1,000 nM, less than 500 nM).

In some aspects, the PDGFRα inhibitor comprises a compound of formula (II) that is capable of (and in some embodiments used for) each of the following: (i) achieving a brain to plasma ratio of greater than 0.1 when systemically administered to a subject; (ii) promoting the differentiation of an OPC into an oligodendrocyte; and (iii) promoting the expression of one or more proteins associated with oligodendrocyte differentiation and/or myelination (e.g., GPR17 and/or MBP). In some aspects, the PDGFRα inhibitor comprises a compound of formula (II) that is capable of (and in some embodiments used for) each of the following: (i) achieving a brain to plasma ratio of greater than 0.1 when systemically administered to a subject; (ii) promoting the differentiation of an OPC into an oligodendrocyte; and (iii) promoting the myelination of an axon. In some aspects, the PDGFRα inhibitor comprises a compound of formula (II) that is capable of (and in some embodiments used for) each of the following: (i) achieving a brain to plasma ratio of greater than 0.1 when systemically administered to a subject; (ii) promoting the differentiation of an OPC into an oligodendrocyte; and (iii) promoting the remyelination of a demyelinated neuronal axon. In some aspects, the PDGFRα inhibitor comprises a compound of formula (II) that is capable of (and in some embodiments used for) each of the following: (i) achieving a brain to plasma ratio of greater than 0.1 when systemically administered to a subject; (ii) promoting the differentiation of an OPC into an oligodendrocyte; and (iii) inhibiting PDGFRα kinase activity with an IC₅₀ of less than 10,000 nM (e.g., less than 1,000 nM, less than 500 nM). In some aspects, the PDGFRα inhibitor comprises a compound of formula (II) that is capable of (and in some embodiments used for) each of the following: (i) achieving a brain to plasma ratio of greater than 0.1 when systemically administered to a subject; (ii) promoting the expression of one or more proteins associated with oligodendrocyte differentiation and/or myelination (e.g., GPR17 and/or MBP); and (iii) promoting the myelination of an axon. In some aspects, the PDGFRα inhibitor comprises a compound of formula (II) that is capable of (and in some embodiments used for) each of the following: (i) achieving a brain to plasma ratio of greater than 0.1 when systemically administered to a subject; (ii) promoting the expression of one or more proteins associated with oligodendrocyte differentiation and/or myelination (e.g., GPR17 and/or MBP); and (iii) promoting the remyelination of a demyelinated neuronal axon. In some aspects, the PDGFRα inhibitor comprises a compound of formula (II) that is capable of (and in some embodiments used for) each of the following: (i) achieving a brain to plasma ratio of greater than 0.1 when systemically administered to a subject; (ii) promoting the expression of one or more proteins associated with oligodendrocyte differentiation and/or myelination (e.g., GPR17 and/or MBP); and (iii) inhibiting PDGFRα kinase activity with an IC₅₀ of less than 10,000 nM (e.g., less than 1,000 nM, less than 500 nM). In some aspects, the PDGFRα inhibitor comprises a compound of formula (II) that is capable of (and in some embodiments used for) each of the following: (i) achieving a brain to plasma ratio of greater than 0.1 when systemically administered to a subject; (ii) promoting the myelination of an axon; and (iii) promoting the remyelination of a demyelinated neuronal axon. In some aspects, the PDGFRα inhibitor comprises a compound of formula (II) that is capable of (and in some embodiments used for) each of the following: (i) achieving a brain to plasma ratio of greater than 0.1 when systemically administered to a subject; (ii) promoting the myelination of an axon; and (iii) inhibiting PDGFRα kinase activity with an IC₅₀ of less than 10,000 nM (e.g., less than 1,000 nM, less than 500 nM). In some aspects, the PDGFRα inhibitor comprises a compound of formula (II) that is capable of (and in some embodiments used for) each of the following: (i) achieving a brain to plasma ratio of greater than 0.1 when systemically administered to a subject; (ii) promoting the remyelination of a demyelinated neuronal axon; and (iii) inhibiting PDGFRα kinase activity with an IC₅₀ of less than 10,000 nM (e.g., less than 1,000 nM, less than 500 nM).

In some aspects, the PDGFRα inhibitor comprises a compound of formula (II) that is capable of (and in some embodiments used for) each of the following: (i) achieving a brain to plasma ratio of greater than 0.1 when systemically administered to a subject; (ii) promoting the differentiation of an OPC into an oligodendrocyte; (iii) promoting the expression of one or more proteins associated with oligodendrocyte differentiation and/or myelination (e.g., GPR17 and/or MBP); and (iv) promoting the myelination of an axon. In some aspects, the PDGFRα inhibitor comprises a compound of formula (II) that is capable of (and in some embodiments used for) each of the following: (i) achieving a brain to plasma ratio of greater than 0.1 when systemically administered to a subject; (ii) promoting the differentiation of an OPC into an oligodendrocyte; (iii) promoting the expression of one or more proteins associated with oligodendrocyte differentiation and/or myelination (e.g., GPR17 and/or MBP); and (iv) promoting the remyelination of a demyelinated neuronal axon. In some aspects, the PDGFRα inhibitor comprises a compound of formula (II) that is capable of (and in some embodiments used for) each of the following: (i) achieving a brain to plasma ratio of greater than 0.1 when systemically administered to a subject; (ii) promoting the differentiation of an OPC into an oligodendrocyte; (iii) promoting the expression of one or more proteins associated with oligodendrocyte differentiation and/or myelination (e.g., GPR17 and/or MBP); and (iv) inhibiting PDGFRα kinase activity with an IC₅₀ of less than 10,000 nM (e.g., less than 1,000 nM, less than 500 nM). In some aspects, the PDGFRα inhibitor comprises a compound of formula (II) that is capable of (and in some embodiments used for) each of the following: (i) achieving a brain to plasma ratio of greater than 0.1 when systemically administered to a subject; (ii) promoting the expression of one or more proteins associated with oligodendrocyte differentiation and/or myelination (e.g., GPR17 and/or MBP); (iii) promoting the myelination of an axon; and (iv) promoting the remyelination of a demyelinated neuronal axon. In some aspects, the PDGFRα inhibitor comprises a compound of formula (II) that is capable of (and in some embodiments used for) each of the following: (i) achieving a brain to plasma ratio of greater than 0.1 when systemically administered to a subject; (ii) promoting the expression of one or more proteins associated with oligodendrocyte differentiation and/or myelination (e.g., GPR17 and/or MBP); (iii) promoting the myelination of an axon; and (iv) inhibiting PDGFRα kinase activity with an IC₅₀ of less than 10,000 nM (e.g., less than 1,000 nM, less than 500 nM). In some aspects, the PDGFRα inhibitor comprises a compound of formula (II) that is capable of (and in some embodiments used for) each of the following: (i) achieving a brain to plasma ratio of greater than 0.1 when systemically administered to a subject; (ii) promoting the myelination of an axon; (iii) promoting the remyelination of a demyelinated neuronal axon; and (iv) inhibiting PDGFRα kinase activity with an IC₅₀ of less than 10,000 nM (e.g., less than 1,000 nM, less than 500 nM).

In some aspects, the PDGFRα inhibitor comprises a compound of formula (II) that is capable of (and in some embodiments used for) each of the following: (i) achieving a brain to plasma ratio of greater than 0.1 when systemically administered to a subject; (ii) promoting the differentiation of an OPC into an oligodendrocyte; (iii) promoting the expression of one or more proteins associated with oligodendrocyte differentiation and/or myelination (e.g., GPR17 and/or MBP); (iv) promoting the myelination of an axon; and (v) promoting the remyelination of a demyelinated neuronal axon. In some aspects, the PDGFRα inhibitor comprises a compound of formula (II) that is capable of (and in some embodiments used for) each of the following: (i) achieving a brain to plasma ratio of greater than 0.1 when systemically administered to a subject; (ii) promoting the differentiation of an OPC into an oligodendrocyte; (iii) promoting the expression of one or more proteins associated with oligodendrocyte differentiation and/or myelination (e.g., GPR17 and/or MBP); (iv) promoting the myelination of an axon; and (v) inhibiting PDGFRα kinase activity with an IC₅₀ of less than 10,000 nM (e.g., less than 1,000 nM, less than 500 nM). In some aspects, the PDGFRα inhibitor comprises a compound of formula (II) that is capable of (and in some embodiments used for) each of the following: (i) achieving a brain to plasma ratio of greater than 0.1 when systemically administered to a subject; (ii) promoting the expression of one or more proteins associated with oligodendrocyte differentiation and/or myelination (e.g., GPR17 and/or MBP); (iii) promoting the myelination of an axon; (iv) promoting the remyelination of a demyelinated neuronal axon; and (v) inhibiting PDGFRα kinase activity with an IC₅₀ of less than 10,000 nM (e.g., less than 1,000 nM, less than 500 nM).

In some aspects, the PDGFRα inhibitor comprises a compound of formula (II) that is capable of (and in some embodiments used for) each of the following: (i) achieving a brain to plasma ratio of greater than 0.1 when systemically administered to a subject; (ii) promoting the differentiation of an OPC into an oligodendrocyte; (iii) promoting the expression of one or more proteins associated with oligodendrocyte differentiation and/or myelination (e.g., GPR17 and/or MBP); (iv) promoting the myelination of an axon; (v) promoting the remyelination of a demyelinated neuronal axon; and (vi) inhibiting PDGFRα kinase activity with an IC₅₀ of less than 10,000 nM (e.g., less than 1,000 nM, less than 500 nM).

In some aspects, a PDGFRα inhibitor that can be used with the present disclosure comprises a compound of formula (III) that is capable of (and in some embodiments used for) achieving a brain to plasma ratio of greater than 0.1 when systemically administered to a subject. In some aspects, the PDGFRα inhibitor comprises a compound of formula (III) that is capable of (and in some embodiments used for) both achieving a brain to plasma ratio of greater than 0.1 when systemically administered to a subject and promoting the differentiation of an OPC into an oligodendrocyte. In some aspects, the PDGFRα inhibitor comprises a compound of formula (III) that is capable of (and in some embodiments used for) both achieving a brain to plasma ratio of greater than 0.1 when systemically administered to a subject and promoting the expression of one or more proteins associated with oligodendrocyte differentiation and/or myelination (e.g., GPR17 and/or MBP). In some aspects, the PDGFRα inhibitor comprises a compound of formula (III) that is capable of (and in some embodiments used for) both achieving a brain to plasma ratio of greater than 0.1 when systemically administered to a subject and promoting the myelination of an axon. In some aspects, the PDGFRα inhibitor comprises a compound of formula (III) that is capable of (and in some embodiments used for) both achieving a brain to plasma ratio of greater than 0.1 when systemically administered to a subject and promoting the remyelination of a demyelinated neuronal axon. In some aspects, the PDGFRα inhibitor comprises a compound of formula (III) that is capable of (and in some embodiments used for) both achieving a brain to plasma ratio of greater than 0.1 when systemically administered to a subject and inhibiting PDGFRα kinase activity with an IC₅₀ of less than 10,000 nM (e.g., less than 1,000 nM, less than 500 nM).

In some aspects, the PDGFRα inhibitor comprises a compound of formula (III) that is capable of (and in some embodiments used for) each of the following: (i) achieving a brain to plasma ratio of greater than 0.1 when systemically administered to a subject; (ii) promoting the differentiation of an OPC into an oligodendrocyte; and (iii) promoting the expression of one or more proteins associated with oligodendrocyte differentiation and/or myelination (e.g., GPR17 and/or MBP). In some aspects, the PDGFRα inhibitor comprises a compound of formula (III) that is capable of (and in some embodiments used for) each of the following: (i) achieving a brain to plasma ratio of greater than 0.1 when systemically administered to a subject; (ii) promoting the differentiation of an OPC into an oligodendrocyte; and (iii) promoting the myelination of an axon. In some aspects, the PDGFRα inhibitor comprises a compound of formula (III) that is capable of (and in some embodiments used for) each of the following: (i) achieving a brain to plasma ratio of greater than 0.1 when systemically administered to a subject; (ii) promoting the differentiation of an OPC into an oligodendrocyte; and (iii) promoting the remyelination of a demyelinated neuronal axon. In some aspects, the PDGFRα inhibitor comprises a compound of formula (III) that is capable of (and in some embodiments used for) each of the following: (i) achieving a brain to plasma ratio of greater than 0.1 when systemically administered to a subject; (ii) promoting the differentiation of an OPC into an oligodendrocyte; and (iii) inhibiting PDGFRα kinase activity with an IC₅₀ of less than 10,000 nM (e.g., less than 1,000 nM, less than 500 nM). In some aspects, the PDGFRα inhibitor comprises a compound of formula (III) that is capable of (and in some embodiments used for) each of the following: (i) achieving a brain to plasma ratio of greater than 0.1 when systemically administered to a subject; (ii) promoting the expression of one or more proteins associated with oligodendrocyte differentiation and/or myelination (e.g., GPR17 and/or MBP); and (iii) promoting the myelination of an axon. In some aspects, the PDGFRα inhibitor comprises a compound of formula (III) that is capable of (and in some embodiments used for) each of the following: (i) achieving a brain to plasma ratio of greater than 0.1 when systemically administered to a subject; (ii) promoting the expression of one or more proteins associated with oligodendrocyte differentiation and/or myelination (e.g., GPR17 and/or MBP); and (iii) promoting the remyelination of a demyelinated neuronal axon. In some aspects, the PDGFRα inhibitor comprises a compound of formula (III) that is capable of (and in some embodiments used for) each of the following: (i) achieving a brain to plasma ratio of greater than 0.1 when systemically administered to a subject; (ii) promoting the expression of one or more proteins associated with oligodendrocyte differentiation and/or myelination (e.g., GPR17 and/or MBP); and (iii) inhibiting PDGFRα kinase activity with an IC₅₀ of less than 10,000 nM (e.g., less than 1,000 nM, less than 500 nM). In some aspects, the PDGFRα inhibitor comprises a compound of formula (III) that is capable of (and in some embodiments used for) each of the following: (i) achieving a brain to plasma ratio of greater than 0.1 when systemically administered to a subject; (ii) promoting the myelination of an axon; and (iii) promoting the remyelination of a demyelinated neuronal axon. In some aspects, the PDGFRα inhibitor comprises a compound of formula (III) that is capable of (and in some embodiments used for) each of the following: (i) achieving a brain to plasma ratio of greater than 0.1 when systemically administered to a subject; (ii) promoting the myelination of an axon; and (iii) inhibiting PDGFRα kinase activity with an IC₅₀ of less than 10,000 nM (e.g., less than 1,000 nM, less than 500 nM). In some aspects, the PDGFRα inhibitor comprises a compound of formula (III) that is capable of (and in some embodiments used for) each of the following: (i) achieving a brain to plasma ratio of greater than 0.1 when systemically administered to a subject; (ii) promoting the remyelination of a demyelinated neuronal axon; and (iii) inhibiting PDGFRα kinase activity with an IC₅₀ of less than 10,000 nM (e.g., less than 1,000 nM, less than 500 nM).

In some aspects, the PDGFRα inhibitor comprises a compound of formula (III) that is capable of (and in some embodiments used for) each of the following: (i) achieving a brain to plasma ratio of greater than 0.1 when systemically administered to a subject; (ii) promoting the differentiation of an OPC into an oligodendrocyte; (iii) promoting the expression of one or more proteins associated with oligodendrocyte differentiation and/or myelination (e.g., GPR17 and/or MBP); and (iv) promoting the myelination of an axon. In some aspects, the PDGFRα inhibitor comprises a compound of formula (III) that is capable of (and in some embodiments used for) each of the following: (i) achieving a brain to plasma ratio of greater than 0.1 when systemically administered to a subject; (ii) promoting the differentiation of an OPC into an oligodendrocyte; (iii) promoting the expression of one or more proteins associated with oligodendrocyte differentiation and/or myelination (e.g., GPR17 and/or MBP); and (iv) promoting the remyelination of a demyelinated neuronal axon. In some aspects, the PDGFRα inhibitor comprises a compound of formula (III) that is capable of (and in some embodiments used for) each of the following: (i) achieving a brain to plasma ratio of greater than 0.1 when systemically administered to a subject; (ii) promoting the differentiation of an OPC into an oligodendrocyte; (iii) promoting the expression of one or more proteins associated with oligodendrocyte differentiation and/or myelination (e.g., GPR17 and/or MBP); and (iv) inhibiting PDGFRα kinase activity with an IC₅₀ of less than 10,000 nM (e.g., less than 1,000 nM, less than 500 nM). In some aspects, the PDGFRα inhibitor comprises a compound of formula (III) that is capable of (and in some embodiments used for) each of the following: (i) achieving a brain to plasma ratio of greater than 0.1 when systemically administered to a subject; (ii) promoting the differentiation of an OPC into an oligodendrocyte; (iii) promoting the expression of one or more proteins associated with oligodendrocyte differentiation and/or myelination (e.g., GPR17 and/or MBP); and (iv) promoting the remyelination of a demyelinated neuronal axon. In some aspects, the PDGFRα inhibitor comprises a compound of formula (III) that is capable of (and in some embodiments used for) each of the following: (i) achieving a brain to plasma ratio of greater than 0.1 when systemically administered to a subject; (ii) promoting the expression of one or more proteins associated with oligodendrocyte differentiation and/or myelination (e.g., GPR17 and/or MBP); (iii) promoting the myelination of an axon; and (iv) inhibiting PDGFRα kinase activity with an IC₅₀ of less than 10,000 nM (e.g., less than 1,000 nM, less than 500 nM). In some aspects, the PDGFRα inhibitor comprises a compound of formula (III) that is capable of (and in some embodiments used for) each of the following: (i) achieving a brain to plasma ratio of greater than 0.1 when systemically administered to a subject; (ii) promoting the myelination of an axon; (iii) promoting the remyelination of a demyelinated neuronal axon; and (iv) inhibiting PDGFRα kinase activity with an IC₅₀ of less than 10,000 nM (e.g., less than 1,000 nM, less than 500 nM).

In some aspects, the PDGFRα inhibitor comprises a compound of formula (III) that is capable of (and in some embodiments used for) each of the following: (i) achieving a brain to plasma ratio of greater than 0.1 when systemically administered to a subject; (ii) promoting the differentiation of an OPC into an oligodendrocyte; (iii) promoting the expression of one or more proteins associated with oligodendrocyte differentiation and/or myelination (e.g., GPR17 and/or MBP); (iv) promoting the myelination of an axon; and (v) promoting the remyelination of a demyelinated neuronal axon. In some aspects, the PDGFRα inhibitor comprises a compound of formula (III) that is capable of (and in some embodiments used for) each of the following: (i) achieving a brain to plasma ratio of greater than 0.1 when systemically administered to a subject; (ii) promoting the differentiation of an OPC into an oligodendrocyte; (iii) promoting the expression of one or more proteins associated with oligodendrocyte differentiation and/or myelination (e.g., GPR17 and/or MBP); (iv) promoting the myelination of an axon; and (v) inhibiting PDGFRα kinase activity with an IC₅₀ of less than 10,000 nM (e.g., less than 1,000 nM, less than 500 nM). In some aspects, the PDGFRα inhibitor comprises a compound of formula (III) that is capable of (and in some embodiments used for) each of the following: (i) achieving a brain to plasma ratio of greater than 0.1 when systemically administered to a subject; (ii) promoting the expression of one or more proteins associated with oligodendrocyte differentiation and/or myelination (e.g., GPR17 and/or MBP); (iii) promoting the myelination of an axon; (iv) promoting the remyelination of a demyelinated neuronal axon; and (v) inhibiting PDGFRα kinase activity with an IC₅₀ of less than 10,000 nM (e.g., less than 1,000 nM, less than 500 nM).

In some aspects, the PDGFRα inhibitor comprises a compound of formula (III) that is capable of (and in some embodiments used for) each of the following: (i) achieving a brain to plasma ratio of greater than 0.1 when systemically administered to a subject; (ii) promoting the differentiation of an OPC into an oligodendrocyte; (iii) promoting the expression of one or more proteins associated with oligodendrocyte differentiation and/or myelination (e.g., GPR17 and/or MBP); (iv) promoting the myelination of an axon; (v) promoting the remyelination of a demyelinated neuronal axon; and (vi) inhibiting PDGFRα kinase activity with an IC₅₀ of less than 10,000 nM (e.g., less than 1,000 nM, less than 500 nM).

In some aspects, a PDGFRα inhibitor that can be used with the present disclosure comprises a compound of formula (IV) that is capable of (and in some embodiments used for) achieving a brain to plasma ratio of greater than 0.1 when systemically administered to a subject. In some aspects, the PDGFRα inhibitor comprises a compound of formula (IV) that is capable of (and in some embodiments used for) both achieving a brain to plasma ratio of greater than 0.1 when systemically administered to a subject and promoting the differentiation of an OPC into an oligodendrocyte. In some aspects, the PDGFRα inhibitor comprises a compound of formula (IV) that is capable of (and in some embodiments used for) both achieving a brain to plasma ratio of greater than 0.1 when systemically administered to a subject and promoting the expression of one or more proteins associated with oligodendrocyte differentiation and/or myelination (e.g., GPR17 and/or MBP). In some aspects, the PDGFRα inhibitor comprises a compound of formula (IV) that is capable of (and in some embodiments used for) both achieving a brain to plasma ratio of greater than 0.1 when systemically administered to a subject and promoting the myelination of an axon. In some aspects, the PDGFRα inhibitor comprises a compound of formula (IV) that is capable of (and in some embodiments used for) both achieving a brain to plasma ratio of greater than 0.1 when systemically administered to a subject and promoting the remyelination of a demyelinated neuronal axon. In some aspects, the PDGFRα inhibitor comprises a compound of formula (IV) that is capable of (and in some embodiments used for) both achieving a brain to plasma ratio of greater than 0.1 when systemically administered to a subject and inhibiting PDGFRα kinase activity with an IC₅₀ of less than 10,000 nM (e.g., less than 1,000 nM, less than 500 nM).

In some aspects, the PDGFRα inhibitor comprises a compound of formula (IV) that is capable of (and in some embodiments used for) each of the following: (i) achieving a brain to plasma ratio of greater than 0.1 when systemically administered to a subject; (ii) promoting the differentiation of an OPC into an oligodendrocyte; and (iii) promoting the expression of one or more proteins associated with oligodendrocyte differentiation and/or myelination (e.g., GPR17 and/or MBP). In some aspects, the PDGFRα inhibitor comprises a compound of formula (IV) that is capable of (and in some embodiments used for) each of the following: (i) achieving a brain to plasma ratio of greater than 0.1 when systemically administered to a subject; (ii) promoting the differentiation of an OPC into an oligodendrocyte; and (iii) promoting the myelination of an axon. In some aspects, the PDGFRα inhibitor comprises a compound of formula (IV) that is capable of (and in some embodiments used for) each of the following: (i) achieving a brain to plasma ratio of greater than 0.1 when systemically administered to a subject; (ii) promoting the differentiation of an OPC into an oligodendrocyte; and (iii) promoting the remyelination of a demyelinated neuronal axon. In some aspects, the PDGFRα inhibitor comprises a compound of formula (IV) that is capable of (and in some embodiments used for) each of the following: (i) achieving a brain to plasma ratio of greater than 0.1 when systemically administered to a subject; (ii) promoting the differentiation of an OPC into an oligodendrocyte; and (iii) inhibiting PDGFRα kinase activity with an IC₅₀ of less than 10,000 nM (e.g., less than 1,000 nM, less than 500 nM). In some aspects, the PDGFRα inhibitor comprises a compound of formula (IV) that is capable of (and in some embodiments used for) each of the following: (i) achieving a brain to plasma ratio of greater than 0.1 when systemically administered to a subject; (ii) promoting the expression of one or more proteins associated with oligodendrocyte differentiation and/or myelination (e.g., GPR17 and/or MBP); and (iii) promoting the myelination of an axon. In some aspects, the PDGFRα inhibitor comprises a compound of formula (IV) that is capable of (and in some embodiments used for) each of the following: (i) achieving a brain to plasma ratio of greater than 0.1 when systemically administered to a subject; (ii) promoting the expression of one or more proteins associated with oligodendrocyte differentiation and/or myelination (e.g., GPR17 and/or MBP); and (iii) promoting the remyelination of a demyelinated neuronal axon. In some aspects, the PDGFRα inhibitor comprises a compound of formula (IV) that is capable of (and in some embodiments used for) each of the following: (i) achieving a brain to plasma ratio of greater than 0.1 when systemically administered to a subject; (ii) promoting the expression of one or more proteins associated with oligodendrocyte differentiation and/or myelination (e.g., GPR17 and/or MBP); and (iii) inhibiting PDGFRα kinase activity with an IC₅₀ of less than 10,000 nM (e.g., less than 1,000 nM, less than 500 nM). In some aspects, the PDGFRα inhibitor comprises a compound of formula (IV) that is capable of (and in some embodiments used for) each of the following: (i) achieving a brain to plasma ratio of greater than 0.1 when systemically administered to a subject; (ii) promoting the myelination of an axon; and (iii) promoting the remyelination of a demyelinated neuronal axon. In some aspects, the PDGFRα inhibitor comprises a compound of formula (IV) that is capable of (and in some embodiments used for) each of the following: (i) achieving a brain to plasma ratio of greater than 0.1 when systemically administered to a subject; (ii) promoting the myelination of an axon; and (iii) inhibiting PDGFRα kinase activity with an IC₅₀ of less than 10,000 nM (e.g., less than 1,000 nM, less than 500 nM). In some aspects, the PDGFRα inhibitor comprises a compound of formula (IV) that is capable of (and in some embodiments used for) each of the following: (i) achieving a brain to plasma ratio of greater than 0.1 when systemically administered to a subject; (ii) promoting the remyelination of a demyelinated neuronal axon; and (iii) inhibiting PDGFRα kinase activity with an IC₅₀ of less than 10,000 nM (e.g., less than 1,000 nM, less than 500 nM).

In some aspects, the PDGFRα inhibitor comprises a compound of formula (IV) that is capable of (and in some embodiments used for) each of the following: (i) achieving a brain to plasma ratio of greater than 0.1 when systemically administered to a subject; (ii) promoting the differentiation of an OPC into an oligodendrocyte; (iii) promoting the expression of one or more proteins associated with oligodendrocyte differentiation and/or myelination (e.g., GPR17 and/or MBP); and (iv) promoting the myelination of an axon. In some aspects, the PDGFRα inhibitor comprises a compound of formula (IV) that is capable of (and in some embodiments used for) each of the following: (i) achieving a brain to plasma ratio of greater than 0.1 when systemically administered to a subject; (ii) promoting the differentiation of an OPC into an oligodendrocyte; (iii) promoting the expression of one or more proteins associated with oligodendrocyte differentiation and/or myelination (e.g., GPR17 and/or MBP); and (iv) promoting the remyelination of a demyelinated neuronal axon. In some aspects, the PDGFRα inhibitor comprises a compound of formula (IV) that is capable of (and in some embodiments used for) each of the following: (i) achieving a brain to plasma ratio of greater than 0.1 when systemically administered to a subject; (ii) promoting the differentiation of an OPC into an oligodendrocyte; (iii) promoting the expression of one or more proteins associated with oligodendrocyte differentiation and/or myelination (e.g., GPR17 and/or MBP); and (iv) inhibiting PDGFRα kinase activity with an IC₅₀ of less than 10,000 nM (e.g., less than 1,000 nM, less than 500 nM). In some aspects, the PDGFRα inhibitor comprises a compound of formula (IV) that is capable of (and in some embodiments used for) each of the following: (i) achieving a brain to plasma ratio of greater than 0.1 when systemically administered to a subject; (ii) promoting the expression of one or more proteins associated with oligodendrocyte differentiation and/or myelination (e.g., GPR17 and/or MBP); (iii) promoting the myelination of an axon; and (iv) promoting the remyelination of a demyelinated neuronal axon. In some aspects, the PDGFRα inhibitor comprises a compound of formula (IV) that is capable of (and in some embodiments used for) each of the following: (i) achieving a brain to plasma ratio of greater than 0.1 when systemically administered to a subject; (ii) promoting the expression of one or more proteins associated with oligodendrocyte differentiation and/or myelination (e.g., GPR17 and/or MBP); (iii) promoting the myelination of an axon; and (iv) inhibiting PDGFRα kinase activity with an IC₅₀ of less than 10,000 nM (e.g., less than 1,000 nM, less than 500 nM). In some aspects, the PDGFRα inhibitor comprises a compound of formula (IV) that is capable of (and in some embodiments used for) each of the following: (i) achieving a brain to plasma ratio of greater than 0.1 when systemically administered to a subject; (ii) promoting the myelination of an axon; (iii) promoting the remyelination of a demyelinated neuronal axon; and (iv) inhibiting PDGFRα kinase activity with an IC₅₀ of less than 10,000 nM (e.g., less than 1,000 nM, less than 500 nM).

In some aspects, the PDGFRα inhibitor comprises a compound of formula (IV) that is capable of (and in some embodiments used for) each of the following: (i) achieving a brain to plasma ratio of greater than 0.1 when systemically administered to a subject; (ii) promoting the differentiation of an OPC into an oligodendrocyte; (iii) promoting the expression of one or more proteins associated with oligodendrocyte differentiation and/or myelination (e.g., GPR17 and/or MBP); (iv) promoting the myelination of an axon; and (v) promoting the remyelination of a demyelinated neuronal axon. In some aspects, the PDGFRα inhibitor comprises a compound of formula (IV) that is capable of (and in some embodiments used for) each of the following: (i) achieving a brain to plasma ratio of greater than 0.1 when systemically administered to a subject; (ii) promoting the differentiation of an OPC into an oligodendrocyte; (iii) promoting the expression of one or more proteins associated with oligodendrocyte differentiation and/or myelination (e.g., GPR17 and/or MBP); (iv) promoting the myelination of an axon; and (v) inhibiting PDGFRα kinase activity with an IC₅₀ of less than 10,000 nM (e.g., less than 1,000 nM, less than 500 nM). In some aspects, the PDGFRα inhibitor comprises a compound of formula (IV) that is capable of (and in some embodiments used for) each of the following: (i) achieving a brain to plasma ratio of greater than 0.1 when systemically administered to a subject; (ii) promoting the expression of one or more proteins associated with oligodendrocyte differentiation and/or myelination (e.g., GPR17 and/or MBP); (iii) promoting the myelination of an axon; (iv) promoting the remyelination of a demyelinated neuronal axon; and (v) inhibiting PDGFRα kinase activity with an IC₅₀ of less than 10,000 nM (e.g., less than 1,000 nM, less than 500 nM).

In some aspects, the PDGFRα inhibitor comprises a compound of formula (IV) that is capable of (and in some embodiments used for) each of the following: (i) achieving a brain to plasma ratio of greater than 0.1 when systemically administered to a subject; (ii) promoting the differentiation of an OPC into an oligodendrocyte; (iii) promoting the expression of one or more proteins associated with oligodendrocyte differentiation and/or myelination (e.g., GPR17 and/or MBP); (iv) promoting the myelination of an axon; (v) promoting the remyelination of a demyelinated neuronal axon; and (vi) inhibiting PDGFRα kinase activity with an IC₅₀ of less than 10,000 nM (e.g., less than 1,000 nM, less than 500 nM).

In some aspects, a PDGFRα inhibitor that can be used with the present disclosure comprises an antagonistic anti-PDGFRα antibody that is capable of (and in some embodiments used for) achieving a brain to plasma ratio of greater than 0.1 when systemically administered to a subject. In some aspects, the PDGFRα inhibitor comprises an antagonistic anti-PDGFRα antibody that is capable of (and in some embodiments used for) both achieving a brain to plasma ratio of greater than 0.1 when systemically administered to a subject and promoting the differentiation of an OPC into an oligodendrocyte. In some aspects, the PDGFRα inhibitor comprises an antagonistic anti-PDGFRα antibody that is capable of (and in some embodiments used for) both achieving a brain to plasma ratio of greater than 0.1 when systemically administered to a subject and promoting the expression of one or more proteins associated with oligodendrocyte differentiation and/or myelination (e.g., GPR17 and/or MBP). In some aspects, the PDGFRα inhibitor comprises an antagonistic anti-PDGFRα antibody that is capable of (and in some embodiments used for) both achieving a brain to plasma ratio of greater than 0.1 when systemically administered to a subject and promoting the myelination of an axon. In some aspects, the PDGFRα inhibitor comprises an antagonistic anti-PDGFRα antibody that is capable of both (and in some embodiments used for) achieving a brain to plasma ratio of greater than 0.1 when systemically administered to a subject and promoting the remyelination of a demyelinated neuronal axon. In some aspects, the PDGFRα inhibitor comprises an antagonistic anti-PDGFRα antibody that is capable of both achieving a brain to plasma ratio of greater than 0.1 when systemically administered to a subject and inhibiting PDGFRα kinase activity with an IC₅₀ of less than 10,000 nM (e.g., less than 1,000 nM, less than 500 nM).

In some aspects, the PDGFRα inhibitor comprises an antagonistic anti-PDGFRα antibody that is capable of (and in some embodiments used for) each of the following: (i) achieving a brain to plasma ratio of greater than 0.1 when systemically administered to a subject; (ii) promoting the differentiation of an OPC into an oligodendrocyte; and (iii) promoting the expression of one or more proteins associated with oligodendrocyte differentiation and/or myelination (e.g., GPR17 and/or MBP). In some aspects, the PDGFRα inhibitor comprises an antagonistic anti-PDGFRα antibody that is capable of (and in some embodiments used for) each of the following: (i) achieving a brain to plasma ratio of greater than 0.1 when systemically administered to a subject; (ii) promoting the differentiation of an OPC into an oligodendrocyte; and (iii) promoting the myelination of an axon. In some aspects, the PDGFRα inhibitor comprises an antagonistic anti-PDGFRα antibody that is capable of (and in some embodiments used for) each of the following: (i) achieving a brain to plasma ratio of greater than 0.1 when systemically administered to a subject; (ii) promoting the differentiation of an OPC into an oligodendrocyte; and (iii) promoting the remyelination of a demyelinated neuronal axon. In some aspects, the PDGFRα inhibitor comprises an antagonistic anti-PDGFRα antibody that is capable of (and in some embodiments used for) each of the following: (i) achieving a brain to plasma ratio of greater than 0.1 when systemically administered to a subject; (ii) promoting the differentiation of an OPC into an oligodendrocyte; and (iii) inhibiting PDGFRα kinase activity with an IC₅₀ of less than 10,000 nM (e.g., less than 1,000 nM, less than 500 nM). In some aspects, the PDGFRα inhibitor comprises an antagonistic anti-PDGFRα antibody that is capable of (and in some embodiments used for) each of the following: (i) achieving a brain to plasma ratio of greater than 0.1 when systemically administered to a subject; (ii) promoting the expression of one or more proteins associated with oligodendrocyte differentiation and/or myelination (e.g., GPR17 and/or MBP); and (iii) promoting the myelination of an axon. In some aspects, the PDGFRα inhibitor comprises an antagonistic anti-PDGFRα antibody that is capable of (and in some embodiments used for) each of the following: (i) achieving a brain to plasma ratio of greater than 0.1 when systemically administered to a subject; (ii) promoting the expression of one or more proteins associated with oligodendrocyte differentiation and/or myelination (e.g., GPR17 and/or MBP); and (iii) promoting the remyelination of a demyelinated neuronal axon. In some aspects, the PDGFRα inhibitor comprises an antagonistic anti-PDGFRα antibody that is capable of (and in some embodiments used for) each of the following: (i) achieving a brain to plasma ratio of greater than 0.1 when systemically administered to a subject; (ii) promoting the expression of one or more proteins associated with oligodendrocyte differentiation and/or myelination (e.g., GPR17 and/or MBP); and (iii) inhibiting PDGFRα kinase activity with an IC₅₀ of less than 10,000 nM (e.g., less than 1,000 nM, less than 500 nM). In some aspects, the PDGFRα inhibitor comprises an antagonistic anti-PDGFRα antibody that is capable of (and in some embodiments used for) each of the following: (i) achieving a brain to plasma ratio of greater than 0.1 when systemically administered to a subject; (ii) promoting the myelination of an axon; and (iii) promoting the remyelination of a demyelinated neuronal axon. In some aspects, the PDGFRα inhibitor comprises an antagonistic anti-PDGFRα antibody that is capable of (and in some embodiments used for) each of the following: (i) achieving a brain to plasma ratio of greater than 0.1 when systemically administered to a subject; (ii) promoting the myelination of an axon; and (iii) inhibiting PDGFRα kinase activity with an IC₅₀ of less than 10,000 nM (e.g., less than 1,000 nM, less than 500 nM). In some aspects, the PDGFRα inhibitor comprises an antagonistic anti-PDGFRα antibody that is capable of (and in some embodiments used for) each of the following: (i) achieving a brain to plasma ratio of greater than 0.1 when systemically administered to a subject; (ii) promoting the remyelination of a demyelinated neuronal axon; and (iii) inhibiting PDGFRα kinase activity with an IC₅₀ of less than 10,000 nM (e.g., less than 1,000 nM, less than 500 nM).

In some aspects, the PDGFRα inhibitor comprises an antagonistic anti-PDGFRα antibody that is capable of (and in some embodiments used for) each of the following: (i) achieving a brain to plasma ratio of greater than 0.1 when systemically administered to a subject; (ii) promoting the differentiation of an OPC into an oligodendrocyte; (iii) promoting the expression of one or more proteins associated with oligodendrocyte differentiation and/or myelination (e.g., GPR17 and/or MBP); and (iv) promoting the myelination of an axon. In some aspects, the PDGFRα inhibitor comprises an antagonistic anti-PDGFRα antibody that is capable of (and in some embodiments used for) each of the following: (i) achieving a brain to plasma ratio of greater than 0.1 when systemically administered to a subject; (ii) promoting the differentiation of an OPC into an oligodendrocyte; (iii) promoting the expression of one or more proteins associated with oligodendrocyte differentiation and/or myelination (e.g., GPR17 and/or MBP); and (iv) promoting the remyelination of a demyelinated neuronal axon. In some aspects, the PDGFRα inhibitor comprises an antagonistic anti-PDGFRα antibody that is capable of (and in some embodiments used for) each of the following: (i) achieving a brain to plasma ratio of greater than 0.1 when systemically administered to a subject; (ii) promoting the differentiation of an OPC into an oligodendrocyte; (iii) promoting the expression of one or more proteins associated with oligodendrocyte differentiation and/or myelination (e.g., GPR17 and/or MBP); and (iv) inhibiting PDGFRα kinase activity with an IC₅₀ of less than 10,000 nM (e.g., less than 1,000 nM, less than 500 nM). In some aspects, the PDGFRα inhibitor comprises an antagonistic anti-PDGFRα antibody that is capable of (and in some embodiments used for) each of the following: (i) achieving a brain to plasma ratio of greater than 0.1 when systemically administered to a subject; (ii) promoting the expression of one or more proteins associated with oligodendrocyte differentiation and/or myelination (e.g., GPR17 and/or MBP); (iii) promoting the myelination of an axon; and (iv) promoting the remyelination of a demyelinated neuronal axon. In some aspects, the PDGFRα inhibitor comprises an antagonistic anti-PDGFRα antibody that is capable of (and in some embodiments used for) each of the following: (i) achieving a brain to plasma ratio of greater than 0.1 when systemically administered to a subject; (ii) promoting the expression of one or more proteins associated with oligodendrocyte differentiation and/or myelination (e.g., GPR17 and/or MBP); (iii) promoting the myelination of an axon; and (iv) inhibiting PDGFRα kinase activity with an IC₅₀ of less than 10,000 nM (e.g., less than 1,000 nM, less than 500 nM). In some aspects, the PDGFRα inhibitor comprises an antagonistic anti-PDGFRα antibody that is capable of (and in some embodiments used for) each of the following: (i) achieving a brain to plasma ratio of greater than 0.1 when systemically administered to a subject; (ii) promoting the myelination of an axon; (iii) promoting the remyelination of a demyelinated neuronal axon; and (iv) inhibiting PDGFRα kinase activity with an IC₅₀ of less than 10,000 nM (e.g., less than 1,000 nM, less than 500 nM).

In some aspects, the PDGFRα inhibitor comprises an antagonistic anti-PDGFRα antibody that is capable of (and in some embodiments used for) each of the following: (i) achieving a brain to plasma ratio of greater than 0.1 when systemically administered to a subject; (ii) promoting the differentiation of an OPC into an oligodendrocyte; (iii) promoting the expression of one or more proteins associated with oligodendrocyte differentiation and/or myelination (e.g., GPR17 and/or MBP); (iv) promoting the myelination of an axon; and (v) promoting the remyelination of a demyelinated neuronal axon. In some aspects, the PDGFRα inhibitor comprises an antagonistic anti-PDGFRα antibody that is capable of (and in some embodiments used for) each of the following: (i) achieving a brain to plasma ratio of greater than 0.1 when systemically administered to a subject; (ii) promoting the differentiation of an OPC into an oligodendrocyte; (iii) promoting the expression of one or more proteins associated with oligodendrocyte differentiation and/or myelination (e.g., GPR17 and/or MBP); (iv) promoting the myelination of an axon; and (v) inhibiting PDGFRα kinase activity with an IC₅₀ of less than 10,000 nM (e.g., less than 1,000 nM, less than 500 nM). In some aspects, the PDGFRα inhibitor comprises an antagonistic anti-PDGFRα antibody that is capable of (and in some embodiments used for) each of the following: (i) achieving a brain to plasma ratio of greater than 0.1 when systemically administered to a subject; (ii) promoting the expression of one or more proteins associated with oligodendrocyte differentiation and/or myelination (e.g., GPR17 and/or MBP); (iii) promoting the myelination of an axon; (iv) promoting the remyelination of a demyelinated neuronal axon; and (v) inhibiting PDGFRα kinase activity with an IC₅₀ of less than 10,000 nM (e.g., less than 1,000 nM, less than 500 nM).

In some aspects, the PDGFRα inhibitor comprises an antagonistic anti-PDGFRα antibody that is capable of (and in some embodiments used for) each of the following: (i) achieving a brain to plasma ratio of greater than 0.1 when systemically administered to a subject; (ii) promoting the differentiation of an OPC into an oligodendrocyte; (iii) promoting the expression of one or more proteins associated with oligodendrocyte differentiation and/or myelination (e.g., GPR17 and/or MBP); (iv) promoting the myelination of an axon; (v) promoting the remyelination of a demyelinated neuronal axon; and (vi) inhibiting PDGFRα kinase activity with an IC₅₀ of less than 10,000 nM (e.g., less than 1,000 nM, less than 500 nM).

### Other Properties

In addition to the properties described above, a PDGFRα inhibitor described herein can comprise one or more additional features that are not present in other inhibitors known in the art. As is apparent from the present disclosure, such additional features can be useful in various clinical settings, e.g., to treat a demyelinating disease, such as those described herein.

As described elsewhere in the present disclosure, some exemplary PDGFRα inhibitors of the present disclosure are capable of have greater therapeutic effect than inhibitors known in the art, e.g., when an inhibitor known in the art and an exemplary inhibitor provided herein are assayed against a vehicle control. For example, in some aspects, a PDGFRα inhibitor can have greater potency compared to inhibitors known in the art. In some aspects, a PDGFRα inhibitor can better penetrate the CNS (e.g., can more effectively pass across the blood-brain barrier). Accordingly, in some aspects, PDGFRα inhibitors described herein are more effective at promoting OPC differentiation as compared to inhibitors known in the art. For example, in some aspects, a PDGFRα inhibitor of the present disclosure can achieve greater than 2-fold increase in GPR17 expression compared to a vehicle control at a dose of less than 50 mg/kg, as measured using an in vivo GPR17 assay (such as that described in Example 242). In some aspects, a PDGFRα inhibitor of the present disclosure can achieve greater than 2-fold increase in GPR17 expression compared to the vehicle control at a dose of less than 40 mg/kg, less than 30 mg/kg, less than 20 mg/kg, less than 10 mg/kg, less than 5 mg/kg, less than 4 mg/kg, less than 3 mg/kg, less than 2 mg/kg, or less than 1 mg/kg, as measured using an in vivo GPR17 assay (such as that described in Example 242). As described elsewhere in the present disclosure, GPR17 expression is a suitable marker for OPC differentiation. Additionally, in some aspects, any of the other proteins associated with oligodendrocyte differentiation and/or myelination can be used instead of GPR17 or in combination with GPR17. Non-limiting examples of such proteins include: MBP, ASPA, GST-pi, CC1, MOG, oligodendrocyte-specific protein/claudin-11, CNPase, or a combination thereof.

### II.A. Small Molecules

In some aspects, a PDGFRα inhibitor useful for the present disclosure comprises a small molecule.

Non-limiting examples of such a small molecules include: axitinib and analogs thereof (e.g., as described by Formula I in U.S. Pat. No. 6,534,524), dasatinib and analogs thereof (e.g., as described by Formula I in U.S. Pat. No. 6,596,746), imatinib and analogs thereof (e.g., as described by Formula I in U.S. Pat. No. 5,521,184), lenvatinib and analogs thereof (e.g., as described by Formula I in U.S. Pat. No. 7,253,286), nilotinib and analogs thereof (e.g., as described by Formula I in U.S. Pat. No. 7,169,791), nintedanib and analogs thereof (e.g., as described by Formula I in U.S. Pat. No. 6,762,180), ponatinib and analogs thereof (e.g., as described by Formula I in U.S. Pat. No. 8,114,874), sorafenib and analogs thereof (e.g., as described by Formula I in U.S. Pat. No. 7,351,834), sunitinib and analogs thereof (e.g., as described by Formula I in U.S. Pat. No. 6,573,293), regorafenib and analogs thereof (e.g., as described by Formula I in U.S. Pat. No. 7,351,834), midostaurin and analogs thereof (e.g., as described by Formula I in U.S. Pat. No. 7,973,031), pazopanib and analogs thereof (e.g., as described by Formula I in U.S. Pat. No. 7,105,530), erdafitinib and analogs thereof (e.g., as described by Formula I in U.S. Pat. No. 8,895,601), fostamatinib and analogs thereof (e.g., as described by Formula I in U.S. Pat. No. 7,538,108), tivozanib and analogs thereof (e.g., as described by Formula I in U.S. Pat. No. 6,821,987), avapritinib and analogs thereof (e.g., as described by Formula I in U.S. Pat. No. 9,200,002), amuvatinib and analogs thereof (e.g., as described Structure I in U.S. 2009/0099165), cediranib and analogs thereof (e.g., as described by Formula I in U.S. Pat. No. 7,074,800), crenolanib and analogs thereof (e.g., as described by Formula I in U.S. Pat. No. 7,071,337), dovitinib and analogs thereof (e.g., as described by Structure I in U.S. Pat. No. 6,605,617), lucitanib and analogs thereof (e.g., as described by Formula I in U.S. Pat. No. 8,163,923), masitinib and analogs thereof (e.g., as described by Formula I in U.S. Pat. No. 8,993,573), motesanib and analogs thereof (e.g., as described by Formula I in U.S. Pat. No. 6,995,162), tandutinib and analogs thereof (e.g., as described by Formula I in U.S. Pat. No. 6,982,266), telatinib and analogs thereof (e.g., as described by Formula I in WO 2001/023375), vatalanib and analogs thereof, DMPQ dihydrochloride and analogs thereof, toceranib and analogs thereof (e.g., as described by Formula I in U.S. Pat. No. 7,211,600), tyrphostin A23 and analogs thereof (e.g., as described by Formula I in U.S. RE38,761), apatinib (e.g., as described by Formula I in U.S. Pat. No. 7,129,252), CP-673451 (e.g., as described by Formula I in U.S. Pat. No. 7,019,147), flumatinib mesylate and analogs thereof (e.g., as described by Formula I in U.S. Pat. No. 8,183,242), linifanib and analogs thereof (e.g., as described by Formula I in WO 2004/113304), MK-2461 and analogs thereof (e.g., as described by Formula I in U.S. Pat. No. 7,550,478), PP121 and analogs thereof, sennoside B and analogs thereof, SU 16f and analogs thereof (e.g., as described by Structure 1 in U.S. Pat. No. 6,395,734), SU 4312 and analogs thereof (e.g., as described by Formula I in U.S. Pat. No. 5,834,504), SU 5402 and analogs thereof (e.g., as described by Structure 1 in U.S. Pat. No. 6,531,502), orantinib and analogs thereof (e.g., as described by Formula I in U.S. Pat. No. 7,189,721), malonoben and analogs thereof, tyrphostin AG 1296 and analogs thereof, ENMD-2076 and analogs thereof (e.g., as described by Formula I in U.S. Pat. No. 7,563,787), K252^{a} and analogs thereof (e.g., as described by Formula I in U.S. Pat. No. 4,923,986), bemcentinib and analogs thereof (e.g., as described by Formula I in U.S. Pat. No. 7,709,482), JNJ=28312141 and analogs thereof (e.g., as described by Formula I in U.S. Pat. No. 6,995,162), seralutinib and analogs thereof (e.g., as described by Structure 1 in U.S. Pat. No. 9,815,815), pexidartinib and analogs thereof (e.g., as described by Formula I in U.S. Pat. No. 7,893,075), RO4396686 and analogs thereof (e.g., as described by Formula I in U.S. Pat. No. 7,115,740), JNJ=10198409 and analogs thereof (e.g., as described by Formula I in U.S. Pat. No. 7,196,110), SU-14813 and analogs thereof (e.g., as described by Formula I in U.S. Pat. No. 6,653,308), tamatinib and analogs thereof (e.g., as described by Formula I in U.S. Pat. No. 7,449,458), TAK-593 and analogs thereof (e.g., as described by Formula I in U.S. Pat. No. 8,273,741), anlotinib and analogs thereof (e.g., as described by Formula I in U.S. Pat. No. 8,148,532), ON123300 and analogs thereof (e.g., as described by Formula I in U.S. Pat. No. 8,987,267), foretinib and analogs thereof (e.g., as described by Formula I in U.S. Pat. No. 9,174,947), amcasertib and analogs thereof (e.g., as described by Formula I in U.S. Pat. No. 8,299,106), brivanib and analogs thereof (e.g., as described by Formula I in U.S. Pat. No. 7,521,450), neratinib and analogs thereof (e.g., as described by Formula I in U.S. Pat. No. 6,288,082), PLX-5622 and analogs thereof (e.g., as described in U.S. Pat. No. 9,096,593), N3-(4-(4-cyclohexylpiperazin-1-yl)phenyl)-1-(isoquinolin-1-yl)-1H-1,2,4-triazole-3,5-diamine (e.g., as described as Compound 81 in U.S. Pat. No. 8,906,922; hereinafter referred to as “R81”), combinations thereof, or pharmaceutically acceptable salts or solvates thereof. In some examples, the compound is a prodrug. Additional examples are known in the art. See, e.g., Roskoski, R., Pharmacol Res 129:65-83 (March 2018).

In some aspects, the PDGFRα inhibitor is an imidazo[1,2-b]pyridazine compound, a pyrazolo[1,5-a]pyridine compound, a pyrazolo[1,5-b]pyridazine compound, a pyrazolo[1,5-a]pyrazine compound, a [1,2,4]triazolo[4,3-a]pyridine compound, an imidazo[1,2-a]pyridine compound, an imidazo[1,2-c]pyrimidine compound, an imidazo[1,2-a]pyrimidine compound, a [1,2,3]triazolo[1,5-a]pyridine compound, or a pyrazolo[1,5-a]pyrimidine compound.

In some aspects, the PDGFRα inhibitor is a compound of Formula I:

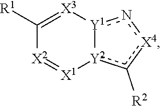

or a pharmaceutically acceptable salt or solvate thereof, wherein:
[symbol] indicates a single bond or a double bond such that all valences are satisfied;
X¹, X², X³, and X⁴ are selected from N and CR^{a}, with the proviso that not more than two of X¹, X², X³, and X⁴ are N;
one of Y¹ and Y² is N and the other of Y¹ and Y² is C;
each R^{a} is independently selected from H, halo, C₁-C₄alkyl, and C₁-C₄alkoxy;
R¹ is selected from C₁-C₄alkyl, C₃-C₈cycloalkyl, 3-8 membered heterocyclyl, heteroaryl, aryl, and C₁-C₈alkoxy, all of which can be optionally substituted with one, two, three, four, five, or six substituents selected from halo, hydroxy, oxo, C₁-C₄alkyl, aminoC₁-C₄alkyl, hydroxyC₁-C₄alkyl, C₁-C₄alkoxy, C₁-C₄alkoxyC₁-C₄alkyl, 3-8 membered heterocyclyl, and 3-8 membered heterocyclylC₁-C₄alkyl, with the proviso that the number of substituents does not exceed the number of substitutable positions;
R² is selected from cycloalkyl, cycloalkenyl, alkyl, aminoalkylamino, amino, heterocyclyl, heteroaryl, aminoheterocyclyl, heterocyclylamino, and aminoalkylamino, all of which can be optionally substituted with one, two, three, four, or five substituents selected from D, halo, oxo, and C₁-C₄alkyl;
R² is substituted by one, two, or three R³;
R³ is selected from aryl, heteroaryl, —C(O)R³¹, —C(O)OR³¹, —C(O)NR³¹R³², —S(O)₂NR³¹R³², —S(O)(NR³³)R³¹, —S(O)(NR³³)NR³¹R³², —C(S)NR³¹R³², C₃-C₈cycloalkyl, 3-8 membered heterocyclyl, and C₁-C₄alkyl, all of which can be optionally substituted with one, two, three, four, or five R³⁰;
each R³⁰ is independently selected from D, halo, aryl, —OR³⁰⁰, —NR³⁰⁰R³⁰¹, —S(O)ᵣR³⁰⁰, —C(O)R³⁰⁰, —C(═CR³⁴R³⁵)R³⁰⁰, and

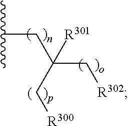

r is selected from 0, 1, and 2;
each R³⁰⁰ is independently selected from C₁-C₆alkyl, C₃-C₇cycloalkyl, aryl, heteroaryl, 3-8 membered heterocyclyl, and 3-8 membered heterocyclylaryl, all of which can be optionally substituted with one, two, three, four, or five substituents selected from D, halo, hydroxy, amino, alkylamino, cyano, C₁-C₄alkyl, C₁-C₄haloalkyl, and C₁-C₄alkoxy;
each R³⁰¹ is independently selected from H, halo, and C₁-C₄alkyl;
each R³⁰² is independently selected from H, F, hydroxy, amino, alkylamino, oxo, and C₁-C₄alkoxy;
each R³⁰³ is independently selected from H and C₁-C₄alkyl;
n, o, and p are each independently selected from 0, 1, 2, 3, and 4;
each R³¹ is independently selected from C₁-C₈alkyl, arylC₁-C₄alkyl, heteroarylC₁-C₄alkyl, heterocyclyl, heterocyclylC₁-C₄alkyl, cycloalkyl, and cycloalkylC₁-C₄alkyl, all of which can be optionally substituted with one, two, three, four, or five substituents selected from D, halo, cyano, hydroxy, amino, —OCF₃, C₁-C₄alkyl, C₁-C₄haloalkyl, C₁-C₄alkoxy, hydroxyC₁-C₄alkyl, —S(O)₂NR³⁰⁴R³⁰⁵, —C(O)OR³⁰⁴R³⁰⁵, —C(O)NR³⁰⁴R³⁰⁵, and —NR³⁰⁴C(O)R³⁰⁵;
each R³⁰⁴ and R³⁰⁵ is independently selected from H and C₁-C₄ alkyl;
each R³² is independently selected from H and C₁-C₄alkyl; or
R³¹ and R³² together with the atom to which they are connected form a 5-8 membered heterocycyl, optionally substituted with one, two, three, four, or five substituents selected from D, halo, cyano, C₁-C₄alkyl, and C₁-C₄haloalkyl, and —C(O)NR³⁴R³⁵;
each R³⁴ and R³⁵ is independently selected from H, C₁-C₄alkyl, and C₁-C₄haloalkyl; and
each R³³ is independently selected from H, C₁-C₄alkyl, C₁-C₄haloalkyl, and —C(O)R³⁴; or
R³¹ and R³³ together with the atoms to which they are connected form a 4-8 membered heterocycyl.

In some aspects, Y¹ is N and Y² is C.

In some aspects, Y¹ is C and Y² is N.

In some aspects, X¹ is N, X² is CR^{a}, X³ is CR^{a}, and X⁴ is CR^{a}.

In some aspects, X¹ is CR^{a}, X² is N, X³ is CR^{a}, and X⁴ is CR^{a}.

In some aspects, X¹ is CR^{a}, X² is CR^{a}, X³ is N, and X⁴ is CR^{a}.

In some aspects, X¹ is CR^{a}, X² is CR^{a}, X³ is CR^{a}, and X⁴ is N.

In some aspects, X¹ is CR^{a}, X² is CR^{a}, X³ is CR^{a}, and X⁴ is CR^{a}.

In some aspects, R¹ is 5- or 6-membered heteroaryl.

In some aspects, R¹ is an optionally substituted pyrazolyl.

In some aspects, R¹ is selected from:

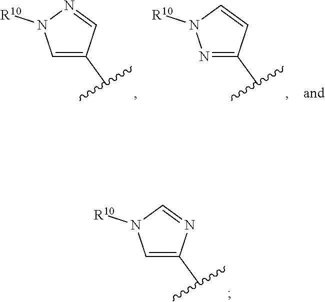

and
R¹⁰ is selected from H, C₁-C₄alkyl, C₁-C₄alkoxy, aminoC₁-C₄alkyl, hydroxyC₁-C₄alkyl, and C₁-C₄alkylsulfonyl, wherein the C₁-C₄alkyl, C₁-C₈alkoxy, aminoC₁-C₄alkyl, hydroxyC₁-C₄alkyl, and C₁-C₄alkylsulfonyl can be optionally substituted by one or more substituents selected from hydroxyl, C₁-C₄alkoxy, NR^{10a}R^{10b}, halo, and deuterium, wherein R^{10a} and R^{10b} are selected from hydrogen and C₁-C₄alkyl, or wherein R^{10a} and R^{10b} taken together with the nitrogen atom to which they are attached form a 4- to 8-membered ring.

In some aspects, R¹ is

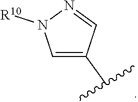

In some aspects, R¹⁰ is CH₃.

In some aspects, R² is heterocyclyl.

In some aspects, R² is selected from:

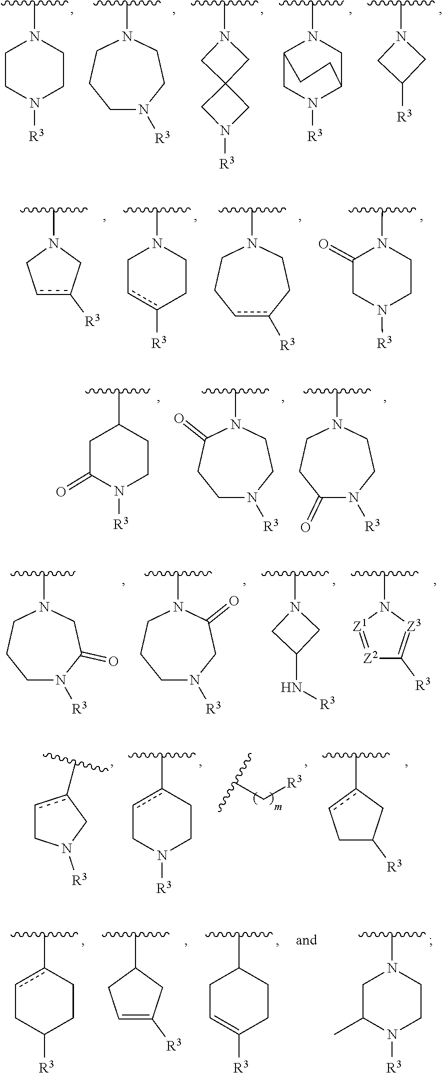

[symbol] indicates a single bond or a double bond such that all valences are satisfied;
m is selected from 0, 1, 2, 3, 4, 5, and 6; and
Z¹, Z², and Z³ are selected from N and CR^{a}.

In some aspects, R² is selected from:

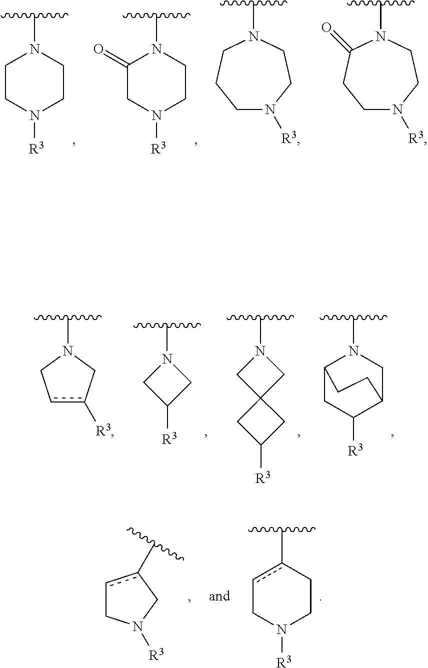

In some aspects, R² is:

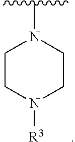

In some aspects, R³ is selected from aryl, heteroaryl, —C(O)R³¹, —C(O)OR³¹, —C(O)NR³¹R³², —S(O)₂NR³¹R³², cycloalkyl, and alkyl, all of which can be optionally substituted with one, two, three, four, or five R³⁰.

In some aspects, R³ is selected from heteroaryl optionally substituted with one, two three, four, or five R³⁰ and —C(O)OR³¹.

In some aspects, the PDGFRα inhibitor is a compound of Formula Ia:

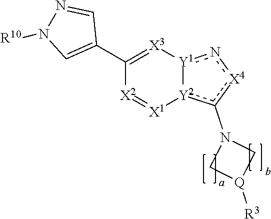

wherein a and b are each independently selected from 1, 2, and 3; and
Q is selected from —CH— and —N—, with the proviso that if Q is —N—, a and b are not 1.

In some aspects, the PDGFRα inhibitor is a compound of Formula II:

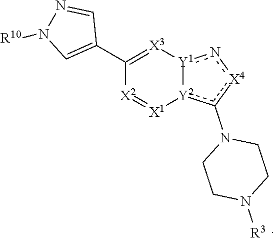

In some aspects, the PDGFRα inhibitor is a compound of Formula IIa:

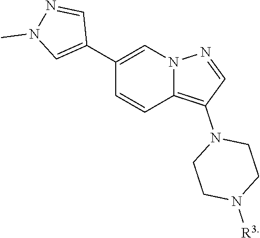

In some aspects, R³ is selected from:

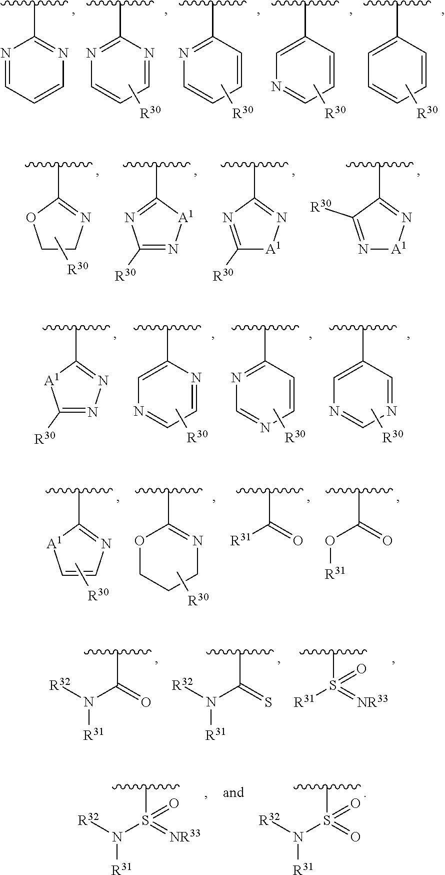

In some aspects, A¹ is selected from O, S, and N.

In some aspects, R³ is selected from:

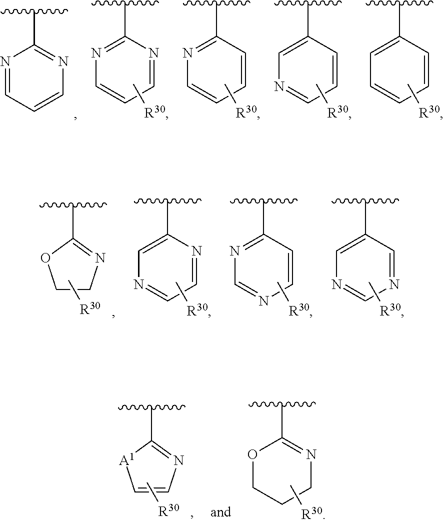

In some aspects, R³ is selected from heteroaryl.

In some aspects, R³ is:

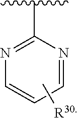

In some aspects, R³ is:

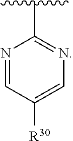

In some aspects, R³⁰ is:

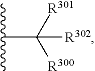

In some aspects, R³⁰⁰ is selected from:

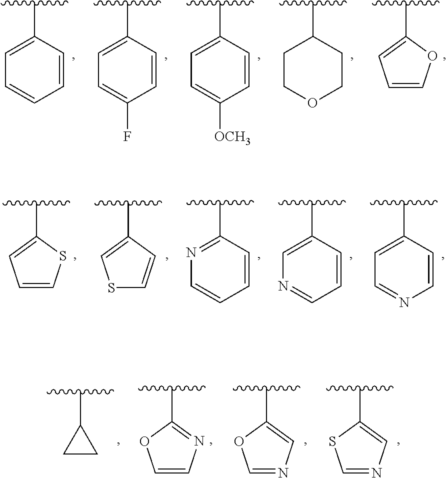

### and H.

In some aspects, R³⁰⁰ is selected from:

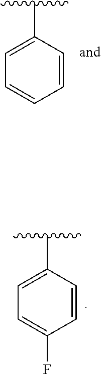 [SMILES: *c1ccccc1 ; *c1ccc(F)cc1]

In some aspects, R³⁰¹ is selected from H and CH₃.

In some aspects, R³⁰¹ is H.

In some aspects, R³⁰² is selected from H and CH₃.

In some aspects, R³⁰² is H.

In some aspects, R³⁰¹ and R³⁰² are H.

In some aspects, R³⁰ is benzyl.

In some aspects, the PDGFRα inhibitor is a compound of Formula III:

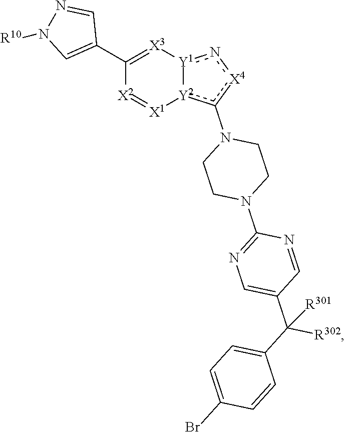

wherein B¹ is H, Cl, F, or —CF₃.

In some aspects, R³ is selected from:

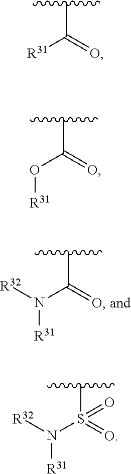

In some aspects, R³ is selected from:

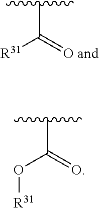

In some aspects, R³ is C(O)OR³¹.

In some aspects, the PDGFRα inhibitor is a compound of Formula IV:

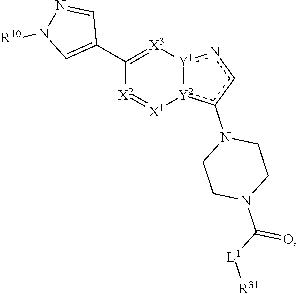

wherein L¹ is a bond or O.

In some aspects, the PDGFRα inhibitor is a compound of Formula IVa:

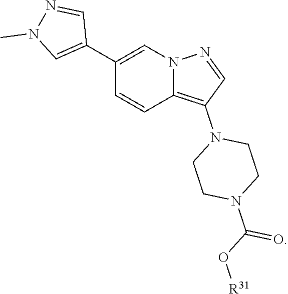

In some aspects, R³¹ is selected from:

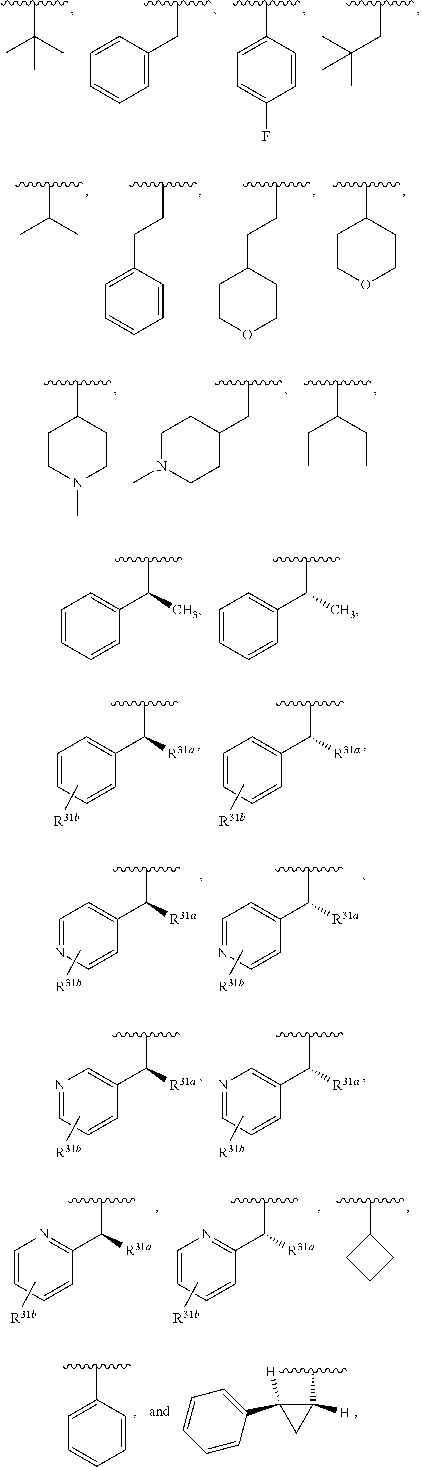

wherein R^{31a} is selected from H, D, halo, hydroxy, amino, alkylamino, C₁-C₄alkyl, and —CF₃; and
R^{31b} is selected from H, D, halo, hydroxy, amino, alkylamino, C₁-C₄alkyl, —CF₃, and —OCF₃.

In some aspects, R³¹ is

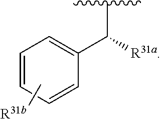

In some aspects, R^{31a} is —CH₃ and R^{31b} is halo, —CH₃, —OCF₃, or CF₃.

In some aspects, R^{31b} is Cl.

In some aspects, R³¹ is

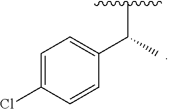 [SMILES: *[C@H](C)c1ccc(Cl)cc1]

Table 1 (below) provides the structure of exemplary small molecules that are useful for the present disclosure (e.g., can target and inhibit kinase activity associated with PDGFRα expressed on OPCs).

TABLE 1
Exemplary Compounds With PDGFRα Inhibitory Activity

| Cmpd. | |
|---|---|
| No. | Structure |
| 1 | 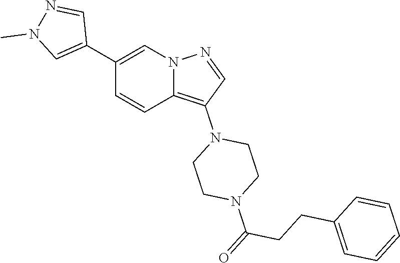 [SMILES: Cn1cc(-c2ccc3c(N4CCN(C(=O)CCc5ccccc5)CC4)cnn3c2)cn1] |
| 2 | 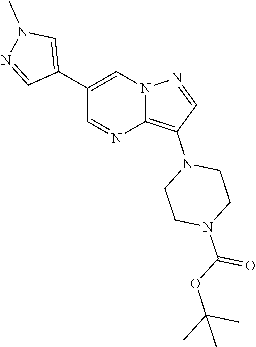 [SMILES: Cn1cc(-c2cnc3c(N4CCN(C(=O)OC(C)(C)C)CC4)cnn3c2)cn1] |
| 3 | 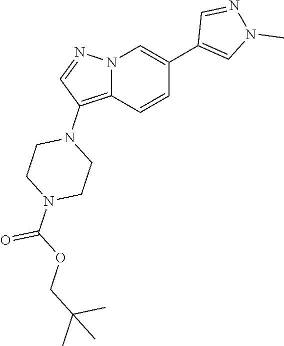 [SMILES: Cn1cc(-c2ccc3c(N4CCN(C(=O)OCC(C)(C)C)CC4)cnn3c2)cn1] |
| 4 | 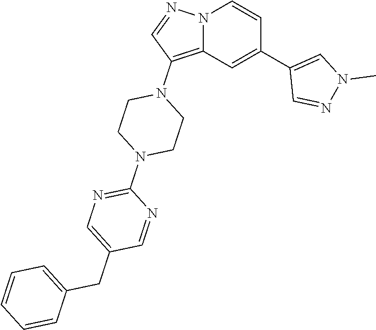 [SMILES: Cn1cc(-c2ccn3ncc(N4CCN(c5ncc(Cc6ccccc6)cn5)CC4)c3c2)cn1] |
| 5 | 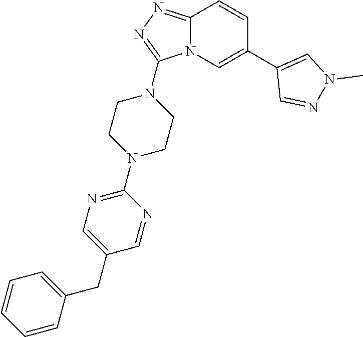 [SMILES: Cn1cc(-c2ccc3nnc(N4CCN(c5ncc(Cc6ccccc6)cn5)CC4)n3c2)cn1] |
| 6 | 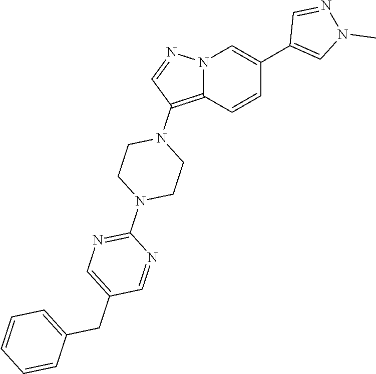 [SMILES: Cn1cc(-c2ccc3c(N4CCN(c5ncc(Cc6ccccc6)cn5)CC4)cnn3c2)cn1] |
| 7 | 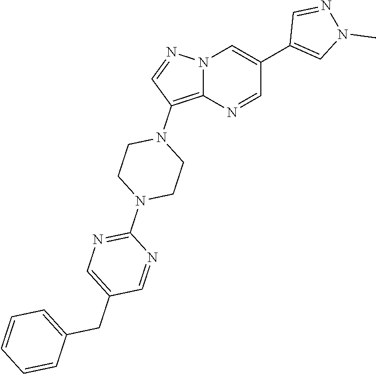 [SMILES: Cn1cc(-c2cnc3c(N4CCN(c5ncc(Cc6ccccc6)cn5)CC4)cnn3c2)cn1] |
| 8 | 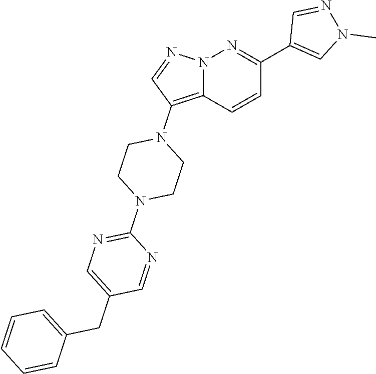 [SMILES: Cn1cc(-c2ccc3c(N4CCN(c5ncc(Cc6ccccc6)cn5)CC4)cnn3n2)cn1] |
| 9 | 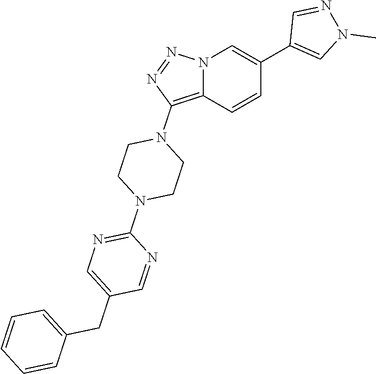 [SMILES: Cn1cc(-c2ccc3c(N4CCN(c5ncc(Cc6ccccc6)cn5)CC4)nnn3c2)cn1] |
| 10 | 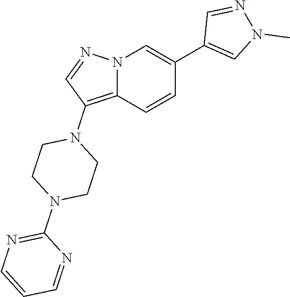 [SMILES: Cn1cc(-c2ccc3c(N4CCN(c5ncccn5)CC4)cnn3c2)cn1] |
| 11 | 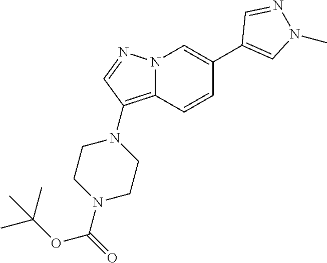 [SMILES: Cn1cc(-c2ccc3c(N4CCN(C(=O)OC(C)(C)C)CC4)cnn3c2)cn1] |
| 12 | 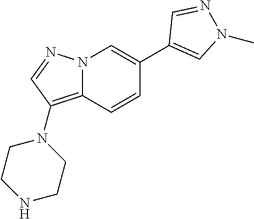 [SMILES: Cn1cc(-c2ccc3c(N4CCNCC4)cnn3c2)cn1] |
| 13 | 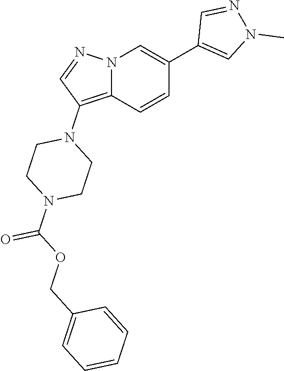 [SMILES: Cn1cc(-c2ccc3c(N4CCN(C(=O)OCc5ccccc5)CC4)cnn3c2)cn1] |
| 14 | 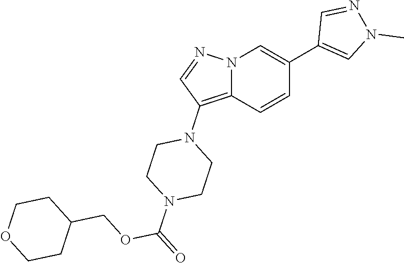 [SMILES: Cn1cc(-c2ccc3c(N4CCN(C(=O)OCC5CCOCC5)CC4)cnn3c2)cn1] |
| 15 | 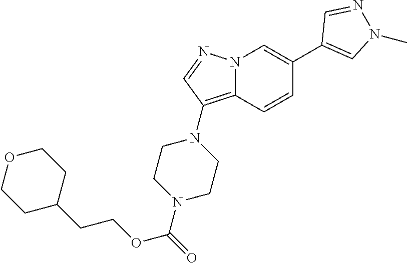 [SMILES: Cn1cc(-c2ccc3c(N4CCN(C(=O)OCCC5CCOCC5)CC4)cnn3c2)cn1] |
| 16 | 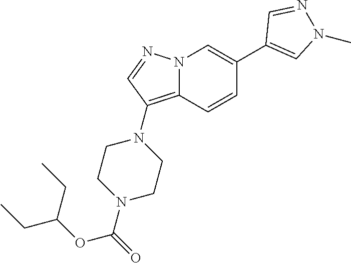 [SMILES: CCC(CC)OC(=O)N1CCN(c2cnn3cc(-c4cnn(C)c4)ccc23)CC1] |
| 17 | 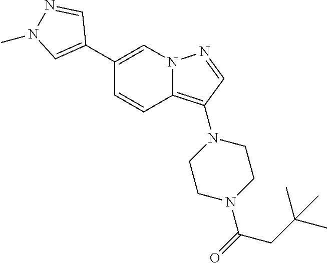 [SMILES: Cn1cc(-c2ccc3c(N4CCN(C(=O)CC(C)(C)C)CC4)cnn3c2)cn1] |
| 18 | 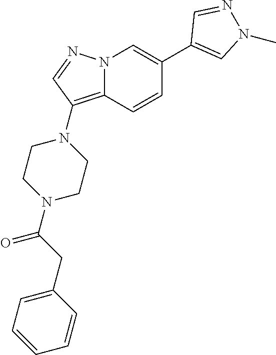 [SMILES: Cn1cc(-c2ccc3c(N4CCN(C(=O)Cc5ccccc5)CC4)cnn3c2)cn1] |
| 19 | 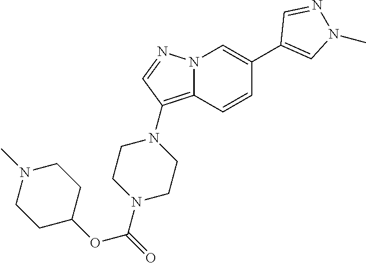 |
| 20 | 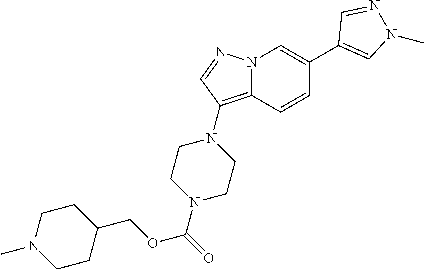 |
| 21 | 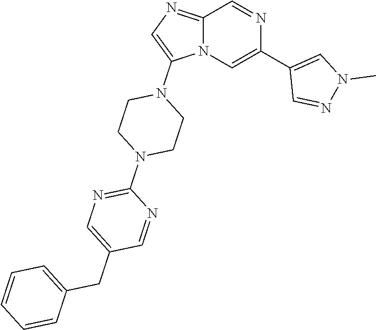 [SMILES: Cn1cc(-c2cn3c(N4CCN(c5ncc(Cc6ccccc6)cn5)CC4)cnc3cn2)cn1] |
| 22 | 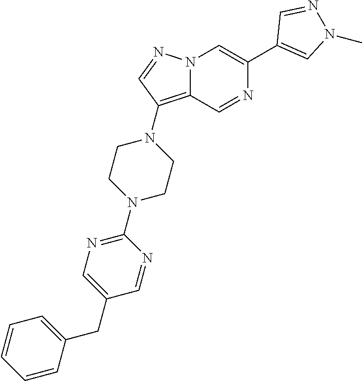 [SMILES: Cn1cc(-c2cn3ncc(N4CCN(c5ncc(Cc6ccccc6)cn5)CC4)c3cn2)cn1] |
| 23 | 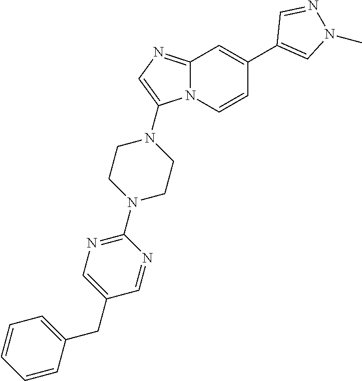 [SMILES: Cn1cc(-c2ccn3c(N4CCN(c5ncc(Cc6ccccc6)cn5)CC4)cnc3c2)cn1] |
| 24 | 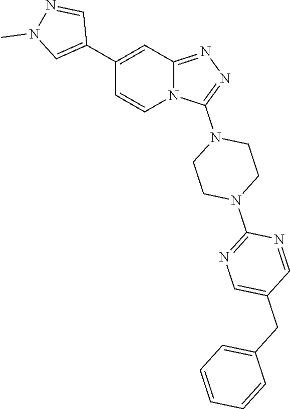 [SMILES: Cn1cc(-c2ccn3c(N4CCN(c5ncc(Cc6ccccc6)cn5)CC4)nnc3c2)cn1] |
| 25 | 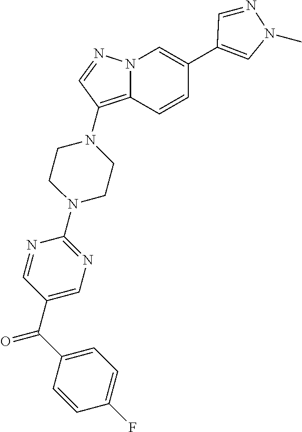 [SMILES: Cn1cc(-c2ccc3c(N4CCN(c5ncc(C(=O)c6ccc(F)cc6)cn5)CC4)cnn3c2)cn1] |
| 26 | 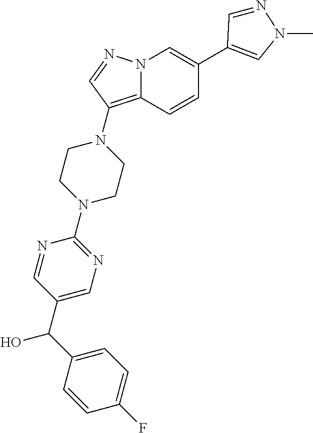 [SMILES: Cn1cc(-c2ccc3c(N4CCN(c5ncc(C(O)c6ccc(F)cc6)cn5)CC4)cnn3c2)cn1] |
| 27 | 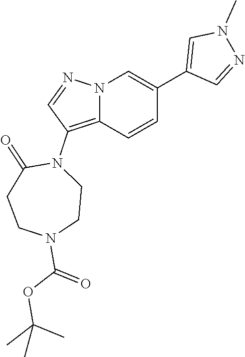 [SMILES: Cn1cc(-c2ccc3c(N4CCN(C(=O)OC(C)(C)C)CCC4=O)cnn3c2)cn1] |
| 28 | 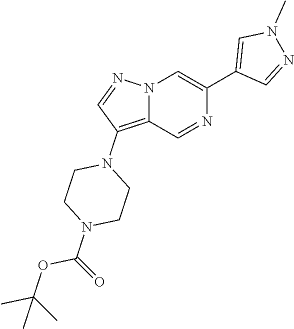 [SMILES: Cn1cc(-c2cn3ncc(N4CCN(C(=O)OC(C)(C)C)CC4)c3cn2)cn1] |
| 29 | 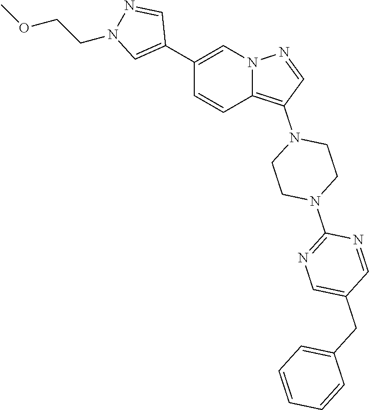 [SMILES: COCCn1cc(-c2ccc3c(N4CCN(c5ncc(Cc6ccccc6)cn5)CC4)cnn3c2)cn1] |
| 30 | 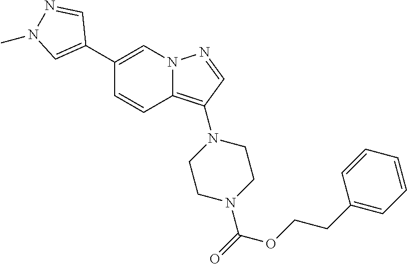 [SMILES: Cn1cc(-c2ccc3c(N4CCN(C(=O)OCCc5ccccc5)CC4)cnn3c2)cn1] |
| 31 | 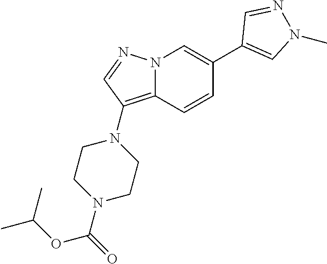 [SMILES: CC(C)OC(=O)N1CCN(c2cnn3cc(-c4cnn(C)c4)ccc23)CC1] |
| 32 | 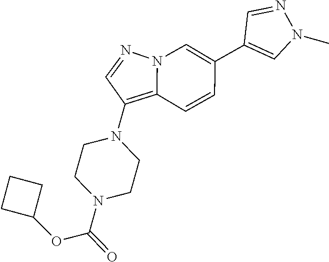 [SMILES: Cn1cc(-c2ccc3c(N4CCN(C(=O)OC5CCC5)CC4)cnn3c2)cn1] |
| 33 | 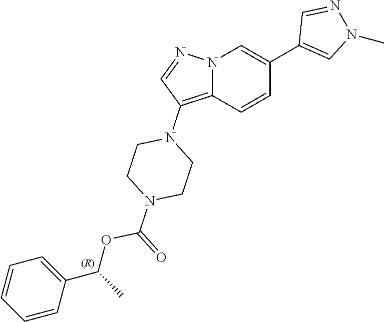 |
| 34 | 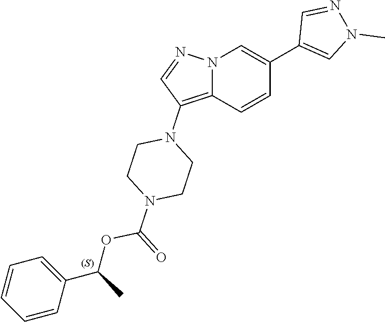 |
| 35 | 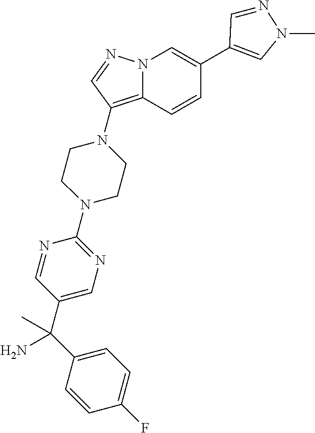 [SMILES: Cn1cc(-c2ccc3c(N4CCN(c5ncc(C(C)(N)c6ccc(F)cc6)cn5)CC4)cnn3c2)cn1] |
| 36 | 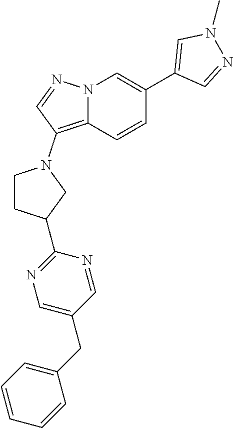 [SMILES: Cn1cc(-c2ccc3c(N4CCC(c5ncc(Cc6ccccc6)cn5)C4)cnn3c2)cn1] |
| 37 | 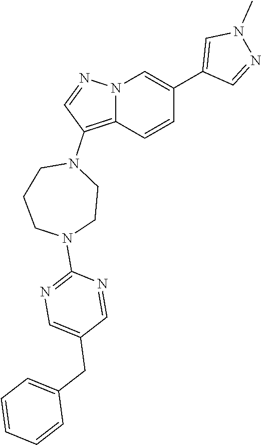 [SMILES: Cn1cc(-c2ccc3c(N4CCCN(c5ncc(Cc6ccccc6)cn5)CC4)cnn3c2)cn1] |
| 38 | 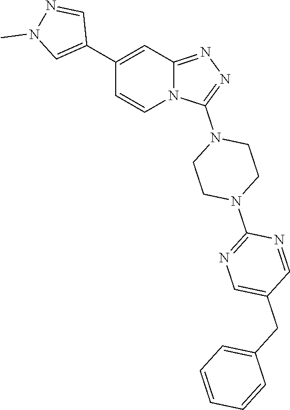 [SMILES: Cn1cc(-c2ccn3c(N4CCN(c5ncc(Cc6ccccc6)cn5)CC4)nnc3c2)cn1] |
| 39 | 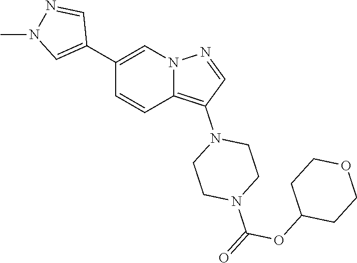 [SMILES: Cn1cc(-c2ccc3c(N4CCN(C(=O)OC5CCOCC5)CC4)cnn3c2)cn1] |
| 40 | 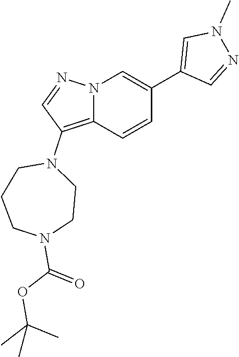 [SMILES: Cn1cc(-c2ccc3c(N4CCCN(C(=O)OC(C)(C)C)CC4)cnn3c2)cn1] |
| 41 | 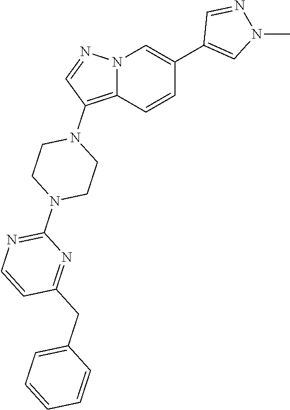 [SMILES: Cn1cc(-c2ccc3c(N4CCN(c5nccc(Cc6ccccc6)n5)CC4)cnn3c2)cn1] |
| 42 | 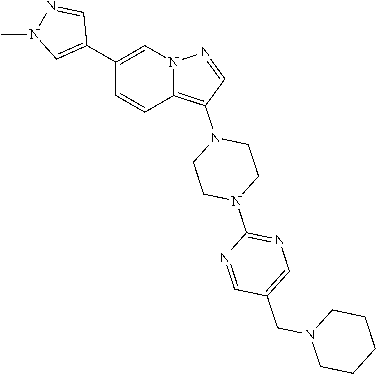 [SMILES: Cn1cc(-c2ccc3c(N4CCN(c5ncc(CN6CCCCC6)cn5)CC4)cnn3c2)cn1] |
| 43 | 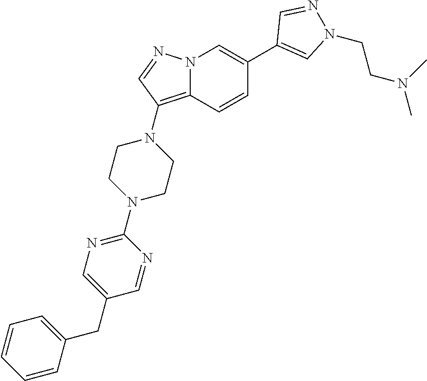 [SMILES: CN(C)CCn1cc(-c2ccc3c(N4CCN(c5ncc(Cc6ccccc6)cn5)CC4)cnn3c2)cn1] |
| 44 | 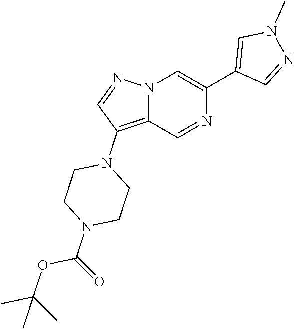 [SMILES: Cn1cc(-c2cn3ncc(N4CCN(C(=O)OC(C)(C)C)CC4)c3cn2)cn1] |
| 45 | 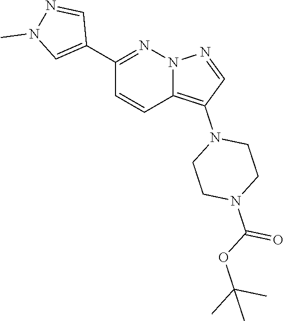 [SMILES: Cn1cc(-c2ccc3c(N4CCN(C(=O)OC(C)(C)C)CC4)cnn3n2)cn1] |
| 46 | 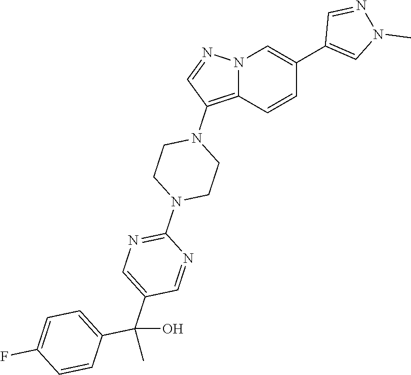 [SMILES: Cn1cc(-c2ccc3c(N4CCN(c5ncc(C(C)(O)c6ccc(F)cc6)cn5)CC4)cnn3c2)cn1] |
| 47 | 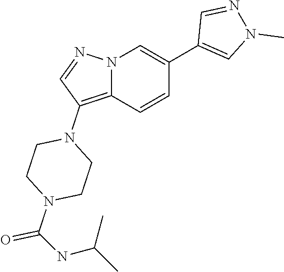 [SMILES: CC(C)NC(=O)N1CCN(c2cnn3cc(-c4cnn(C)c4)ccc23)CC1] |
| 48 | 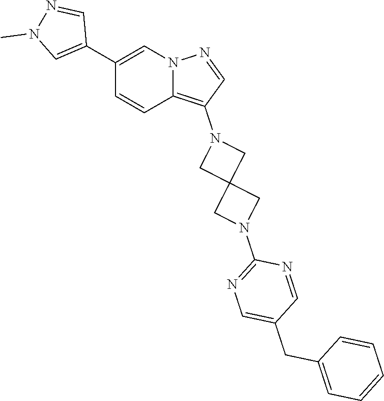 [SMILES: Cn1cc(-c2ccc3c(N4CC5(CN(c6ncc(Cc7ccccc7)cn6)C5)C4)cnn3c2)cn1] |
| 49 | 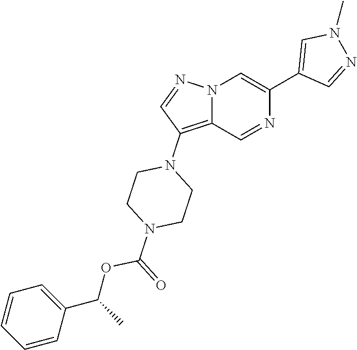 [SMILES: C[C@@H](OC(=O)N1CCN(c2cnn3cc(-c4cnn(C)c4)ncc23)CC1)c1ccccc1] |
| 50 | 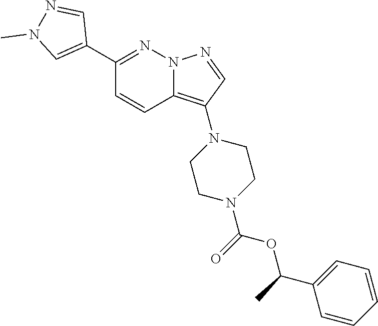 [SMILES: C[C@@H](OC(=O)N1CCN(c2cnn3nc(-c4cnn(C)c4)ccc23)CC1)c1ccccc1] |
| 51 | 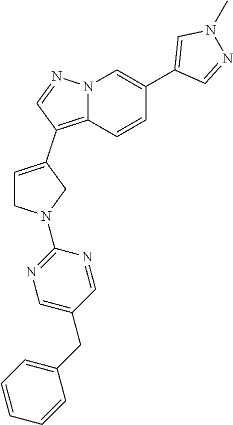 [SMILES: Cn1cc(-c2ccc3c(C4=CCN(c5ncc(Cc6ccccc6)cn5)C4)cnn3c2)cn1] |
| 52 | 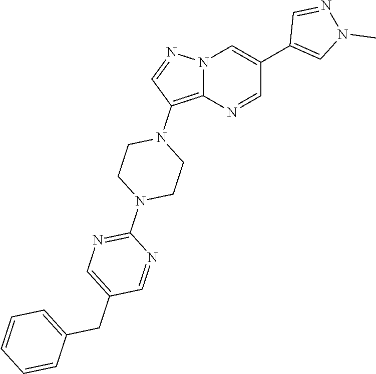 [SMILES: Cn1cc(-c2cnc3c(N4CCN(c5ncc(Cc6ccccc6)cn5)CC4)cnn3c2)cn1] |
| 53 | 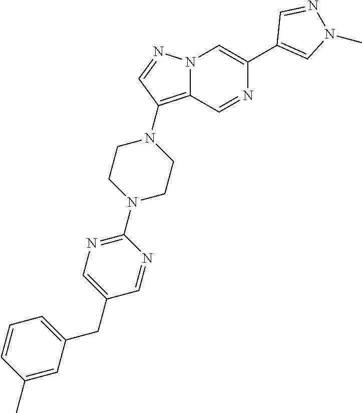 [SMILES: Cc1cccc(Cc2cnc(N3CCN(c4cnn5cc(-c6cnn(C)c6)ncc45)CC3)nc2)c1] |
| 54 | 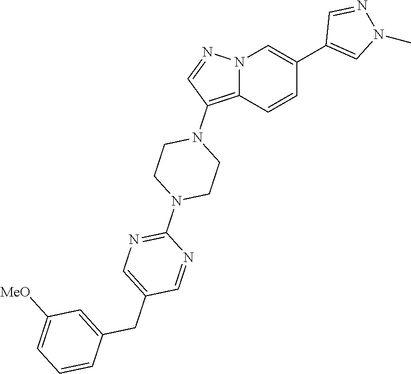 [SMILES: COc1cccc(Cc2cnc(N3CCN(c4cnn5cc(-c6cnn(C)c6)ccc45)CC3)nc2)c1] |
| 55 | 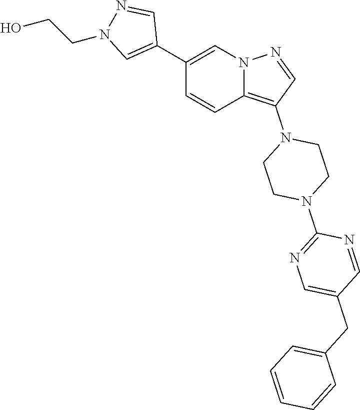 [SMILES: OCCn1cc(-c2ccc3c(N4CCN(c5ncc(Cc6ccccc6)cn5)CC4)cnn3c2)cn1] |
| 56 | 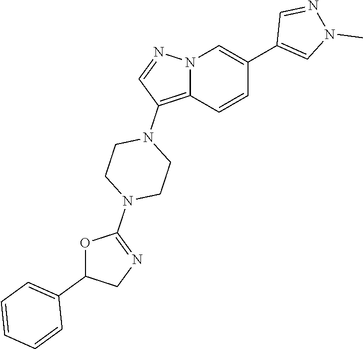 [SMILES: Cn1cc(-c2ccc3c(N4CCN(C5=NCC(c6ccccc6)O5)CC4)cnn3c2)cn1] |
| 57 | 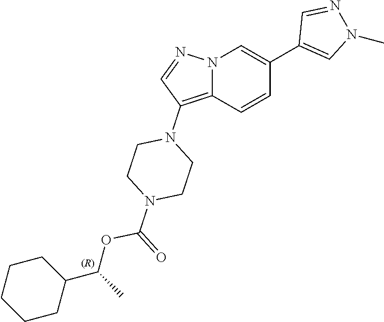 |
| 58 | 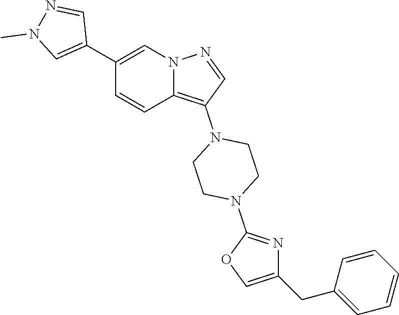 [SMILES: Cn1cc(-c2ccc3c(N4CCN(c5nc(Cc6ccccc6)co5)CC4)cnn3c2)cn1] |
| 59 | 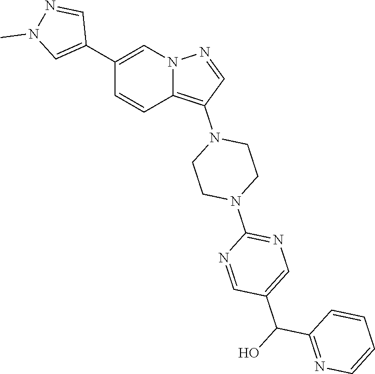 [SMILES: Cn1cc(-c2ccc3c(N4CCN(c5ncc(C(O)c6ccccn6)cn5)CC4)cnn3c2)cn1] |
| 60 | 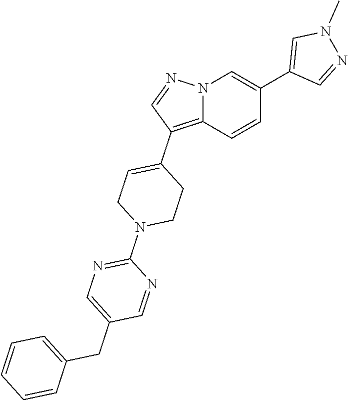 [SMILES: Cn1cc(-c2ccc3c(C4=CCN(c5ncc(Cc6ccccc6)cn5)CC4)cnn3c2)cn1] |
| 61 | 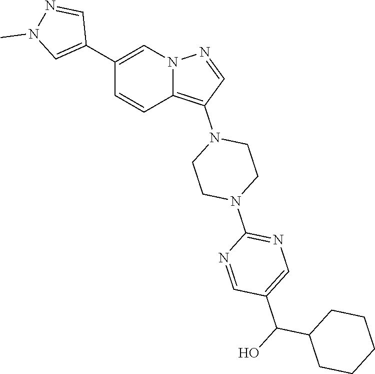 [SMILES: Cn1cc(-c2ccc3c(N4CCN(c5ncc(C(O)C6CCCCC6)cn5)CC4)cnn3c2)cn1] |
| 62 | 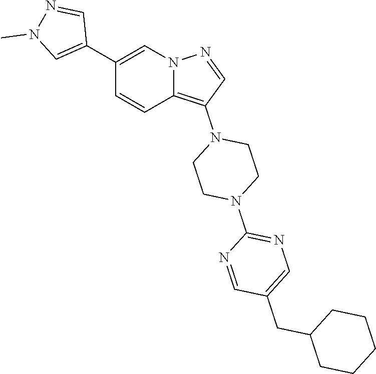 [SMILES: Cn1cc(-c2ccc3c(N4CCN(c5ncc(CC6CCCCC6)cn5)CC4)cnn3c2)cn1] |
| 63 | 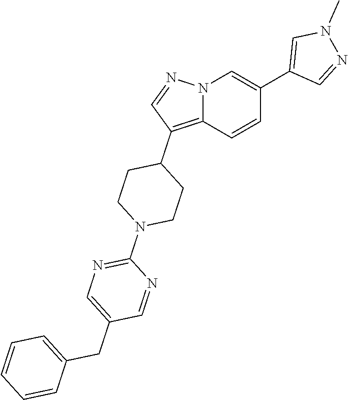 [SMILES: Cn1cc(-c2ccc3c(C4CCN(c5ncc(Cc6ccccc6)cn5)CC4)cnn3c2)cn1] |
| 64 | 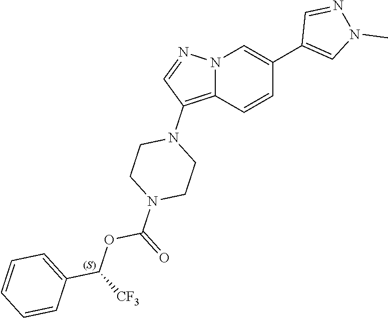 |
| 65 | 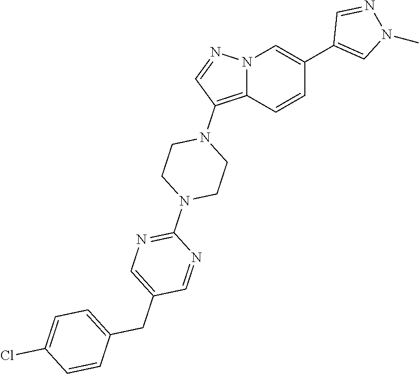 [SMILES: Cn1cc(-c2ccc3c(N4CCN(c5ncc(Cc6ccc(Cl)cc6)cn5)CC4)cnn3c2)cn1] |
| 66 | 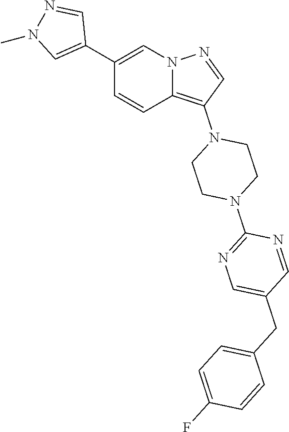 [SMILES: Cn1cc(-c2ccc3c(N4CCN(c5ncc(Cc6ccc(F)cc6)cn5)CC4)cnn3c2)cn1] |
| 67 | 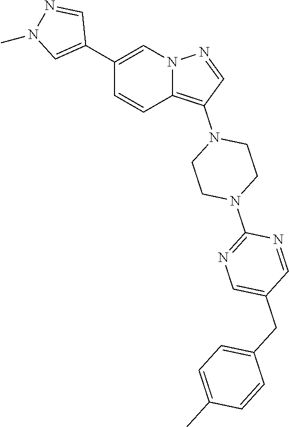 [SMILES: Cc1ccc(Cc2cnc(N3CCN(c4cnn5cc(-c6cnn(C)c6)ccc45)CC3)nc2)cc1] |
| 68 | 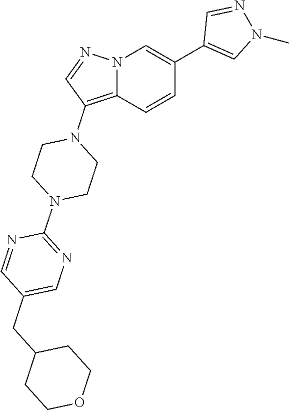 [SMILES: Cn1cc(-c2ccc3c(N4CCN(c5ncc(CC6CCOCC6)cn5)CC4)cnn3c2)cn1] |
| 69 | 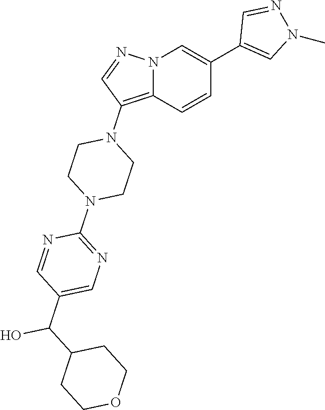 [SMILES: Cn1cc(-c2ccc3c(N4CCN(c5ncc(C(O)C6CCOCC6)cn5)CC4)cnn3c2)cn1] |
| 70 | 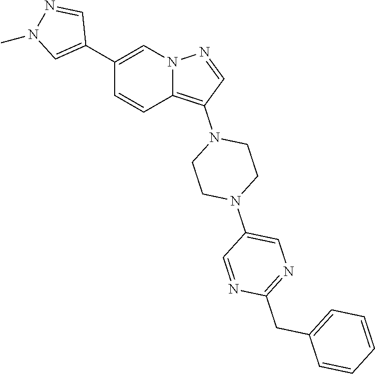 [SMILES: Cn1cc(-c2ccc3c(N4CCN(c5cnc(Cc6ccccc6)nc5)CC4)cnn3c2)cn1] |
| 71 | 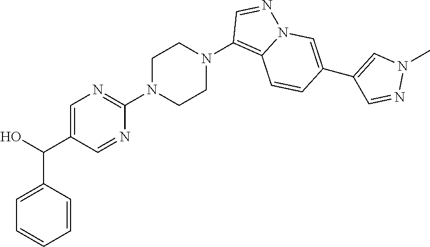 [SMILES: Cn1cc(-c2ccc3c(N4CCN(c5ncc(C(O)c6ccccc6)cn5)CC4)cnn3c2)cn1] |
| 72 | 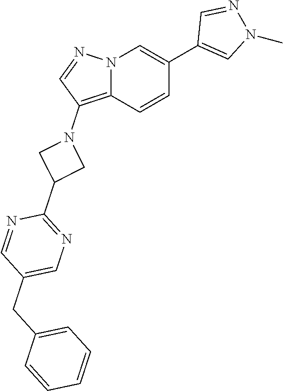 [SMILES: Cn1cc(-c2ccc3c(N4CC(c5ncc(Cc6ccccc6)cn5)C4)cnn3c2)cn1] |
| 73 | 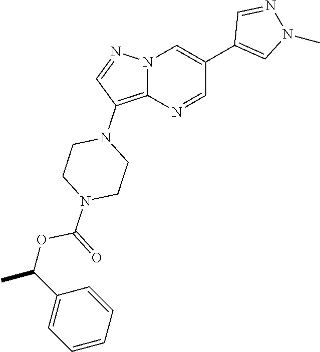 [SMILES: C[C@@H](OC(=O)N1CCN(c2cnn3cc(-c4cnn(C)c4)cnc23)CC1)c1ccccc1] |
| 74 | 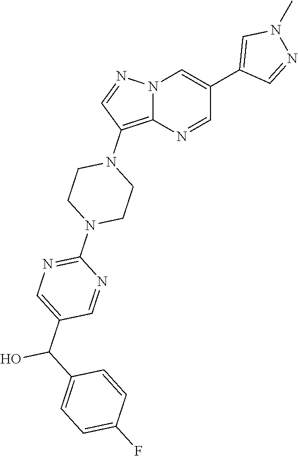 [SMILES: Cn1cc(-c2cnc3c(N4CCN(c5ncc(C(O)c6ccc(F)cc6)cn5)CC4)cnn3c2)cn1] |
| 75 | 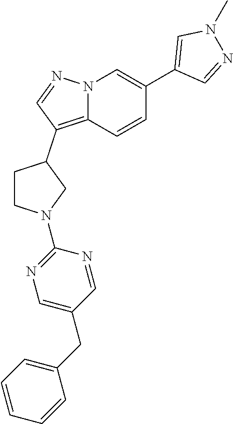 [SMILES: Cn1cc(-c2ccc3c(C4CCN(c5ncc(Cc6ccccc6)cn5)C4)cnn3c2)cn1] |
| 76 | 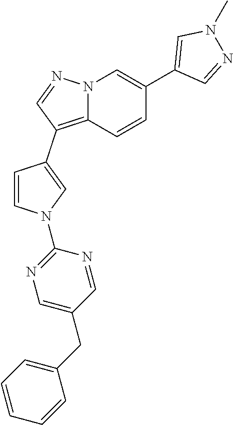 [SMILES: Cn1cc(-c2ccc3c(-c4ccn(-c5ncc(Cc6ccccc6)cn5)c4)cnn3c2)cn1] |
| 77 | 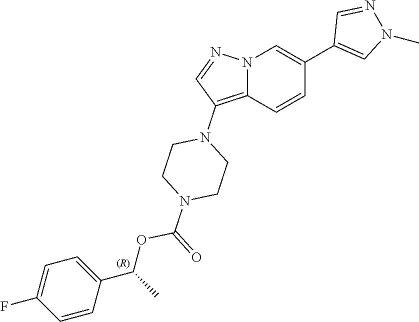 |
| 78 | 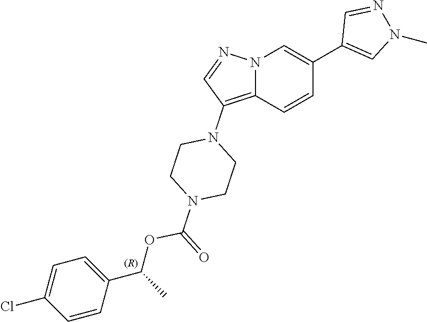 |
| 79 | 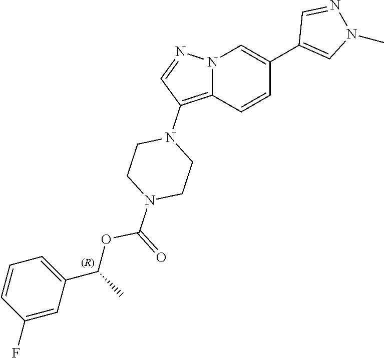 |
| 80 | 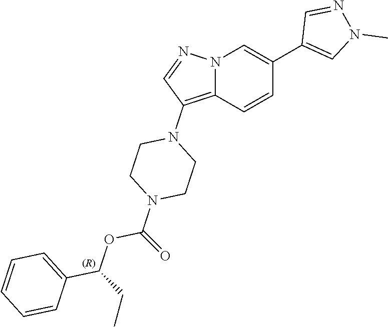 |
| 81 | 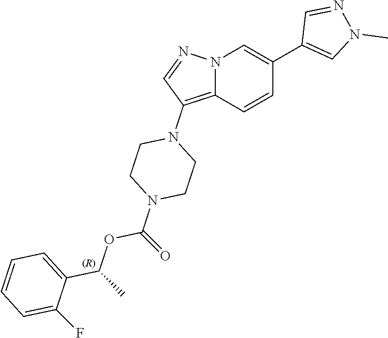 |
| 82 | 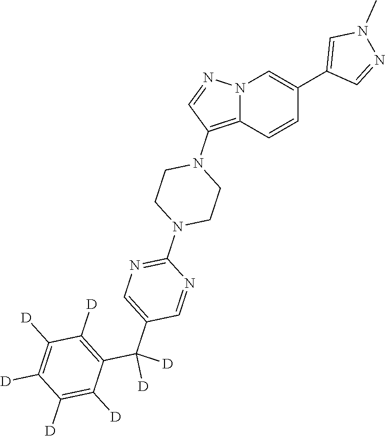 [SMILES: [2H]c1c([2H])c([2H])c(C([2H])([2H])c2cnc(N3CCN(c4cnn5cc(-c6cnn(C)c6)ccc45)CC3)nc2)c([2H])c1[2H]] |
| 83 | 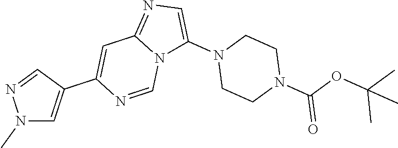 [SMILES: Cn1cc(-c2cc3ncc(N4CCN(C(=O)OC(C)(C)C)CC4)n3cn2)cn1] |
| 84 | 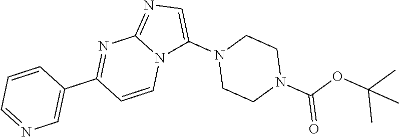 [SMILES: CC(C)(C)OC(=O)N1CCN(c2cnc3nc(-c4cccnc4)ccn23)CC1] |
| 85 | 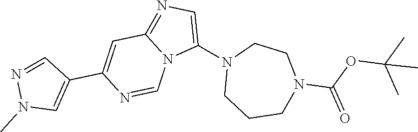 [SMILES: Cn1cc(-c2cc3ncc(N4CCCN(C(=O)OC(C)(C)C)CC4)n3cn2)cn1] |
| 86 | 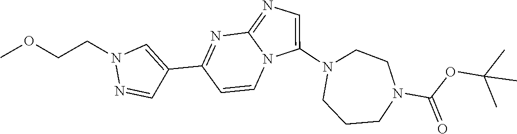 [SMILES: COCCn1cc(-c2ccn3c(N4CCCN(C(=O)OC(C)(C)C)CC4)cnc3n2)cn1] |
| 87 | 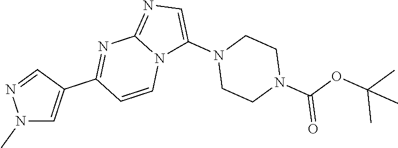 [SMILES: Cn1cc(-c2ccn3c(N4CCN(C(=O)OC(C)(C)C)CC4)cnc3n2)cn1] |
| 88 | 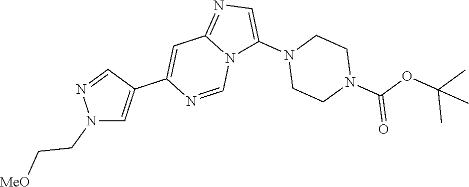 [SMILES: COCCn1cc(-c2cc3ncc(N4CCN(C(=O)OC(C)(C)C)CC4)n3cn2)cn1] |
| 89 | 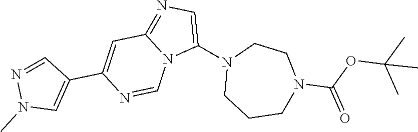 [SMILES: Cn1cc(-c2cc3ncc(N4CCCN(C(=O)OC(C)(C)C)CC4)n3cn2)cn1] |
| 90 | 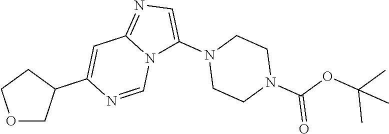 [SMILES: CC(C)(C)OC(=O)N1CCN(c2cnc3cc(C4CCOC4)ncn23)CC1] |
| 91 | 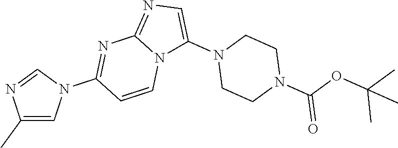 [SMILES: Cc1cn(-c2ccn3c(N4CCN(C(=O)OC(C)(C)C)CC4)cnc3n2)cn1] |
| 92 | 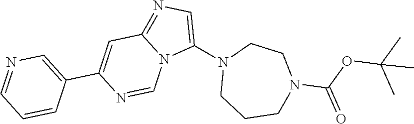 [SMILES: CC(C)(C)OC(=O)N1CCCN(c2cnc3cc(-c4cccnc4)ncn23)CC1] |
| 93 | 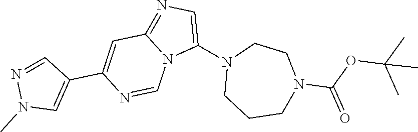 [SMILES: Cn1cc(-c2cc3ncc(N4CCCN(C(=O)OC(C)(C)C)CC4)n3cn2)cn1] |
| 94 | 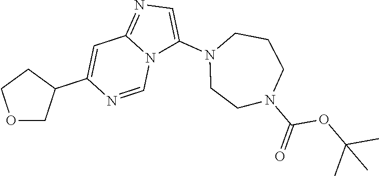 [SMILES: CC(C)(C)OC(=O)N1CCCN(c2cnc3cc(C4CCOC4)ncn23)CC1] |
| 95 | 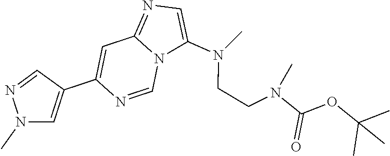 [SMILES: CN(CCN(C)c1cnc2cc(-c3cnn(C)c3)ncn12)C(=O)OC(C)(C)C] |
| 96 | 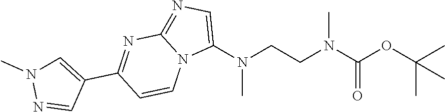 [SMILES: CN(CCN(C)c1cnc2nc(-c3cnn(C)c3)ccn12)C(=O)OC(C)(C)C] |
| 97 | 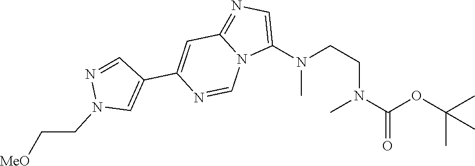 [SMILES: COCCn1cc(-c2cc3ncc(N(C)CCN(C)C(=O)OC(C)(C)C)n3cn2)cn1] |
| 98 | 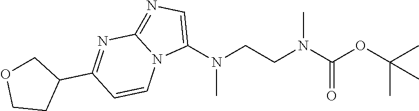 [SMILES: CN(CCN(C)c1cnc2nc(C3CCOC3)ccn12)C(=O)OC(C)(C)C] |
| 99 | 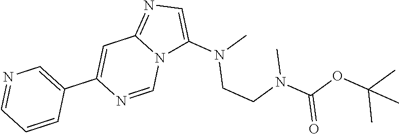 [SMILES: CN(CCN(C)c1cnc2cc(-c3cccnc3)ncn12)C(=O)OC(C)(C)C] |
| 100 | 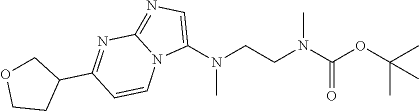 [SMILES: CN(CCN(C)c1cnc2nc(C3CCOC3)ccn12)C(=O)OC(C)(C)C] |
| 101 | 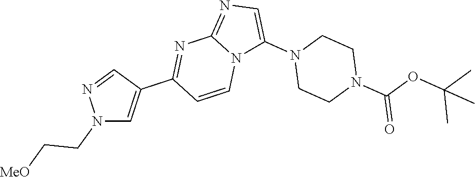 [SMILES: COCCn1cc(-c2ccn3c(N4CCN(C(=O)OC(C)(C)C)CC4)cnc3n2)cn1] |
| 102 | 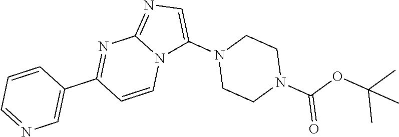 [SMILES: CC(C)(C)OC(=O)N1CCN(c2cnc3nc(-c4cccnc4)ccn23)CC1] |
| 103 | 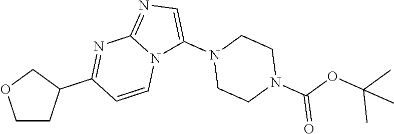 [SMILES: CC(C)(C)OC(=O)N1CCN(c2cnc3nc(C4CCOC4)ccn23)CC1] |
| 104 | 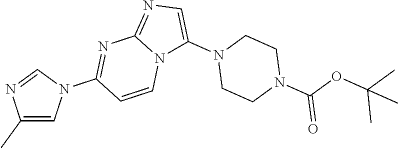 [SMILES: Cc1cn(-c2ccn3c(N4CCN(C(=O)OC(C)(C)C)CC4)cnc3n2)cn1] |
| 105 | 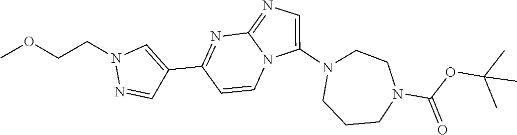 [SMILES: COCCn1cc(-c2ccn3c(N4CCCN(C(=O)OC(C)(C)C)CC4)cnc3n2)cn1] |
| 106 | 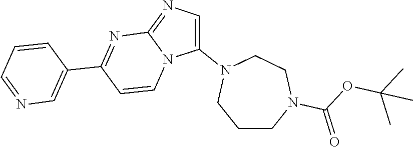 [SMILES: CC(C)(C)OC(=O)N1CCCN(c2cnc3nc(-c4cccnc4)ccn23)CC1] |
| 107 | 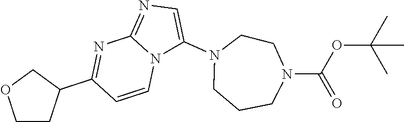 [SMILES: CC(C)(C)OC(=O)N1CCCN(c2cnc3nc(C4CCOC4)ccn23)CC1] |
| 108 | 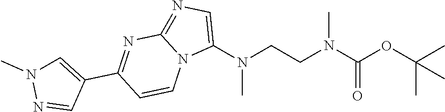 [SMILES: CN(CCN(C)c1cnc2nc(-c3cnn(C)c3)ccn12)C(=O)OC(C)(C)C] |
| 109 | 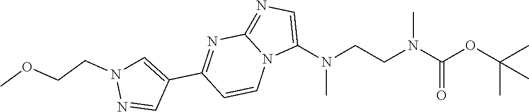 [SMILES: COCCn1cc(-c2ccn3c(N(C)CCN(C)C(=O)OC(C)(C)C)cnc3n2)cn1] |
| 110 | 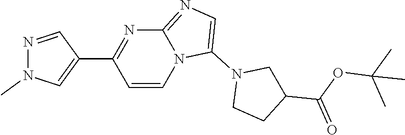 [SMILES: Cn1cc(-c2ccn3c(N4CCC(C(=O)OC(C)(C)C)C4)cnc3n2)cn1] |
| 111 | 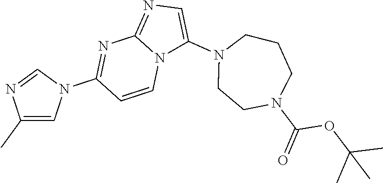 [SMILES: Cc1cn(-c2ccn3c(N4CCCN(C(=O)OC(C)(C)C)CC4)cnc3n2)cn1] |
| 112 | 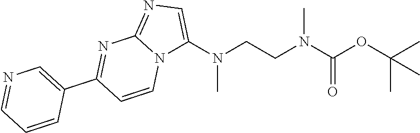 [SMILES: CN(CCN(C)c1cnc2nc(-c3cccnc3)ccn12)C(=O)OC(C)(C)C] |
| 113 | 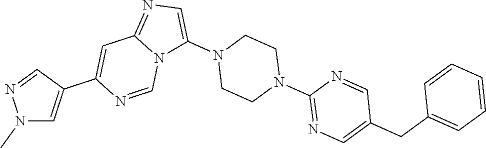 [SMILES: Cn1cc(-c2cc3ncc(N4CCN(c5ncc(Cc6ccccc6)cn5)CC4)n3cn2)cn1] |
| 114 | 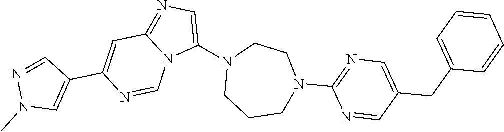 [SMILES: Cn1cc(-c2cc3ncc(N4CCCN(c5ncc(Cc6ccccc6)cn5)CC4)n3cn2)cn1] |
| 115 | 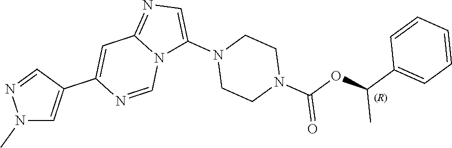 |
| 116 | 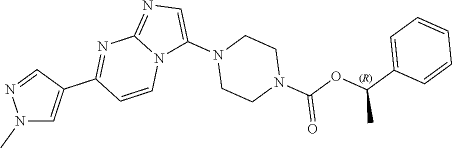 |
| 117 | 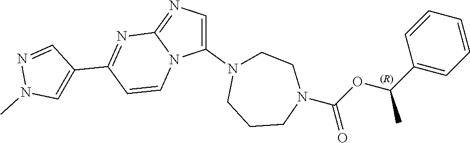 |
| 118 | 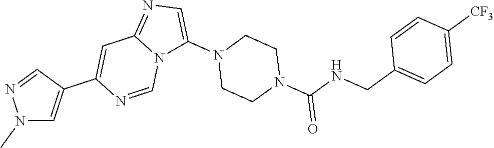 [SMILES: Cn1cc(-c2cc3ncc(N4CCN(C(=O)NCc5ccc(C(F)(F)F)cc5)CC4)n3cn2)cn1] |
| 119 | 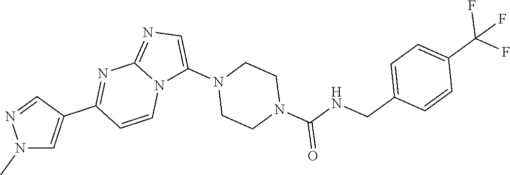 [SMILES: Cn1cc(-c2ccn3c(N4CCN(C(=O)NCc5ccc(C(F)(F)F)cc5)CC4)cnc3n2)cn1] |
| 120 | 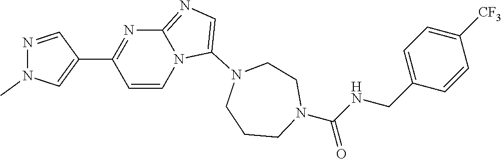 [SMILES: Cn1cc(-c2ccn3c(N4CCCN(C(=O)NCc5ccc(C(F)(F)F)cc5)CC4)cnc3n2)cn1] |
| 121 | 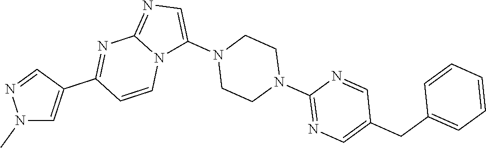 [SMILES: Cn1cc(-c2ccn3c(N4CCN(c5ncc(Cc6ccccc6)cn5)CC4)cnc3n2)cn1] |
| 122 | 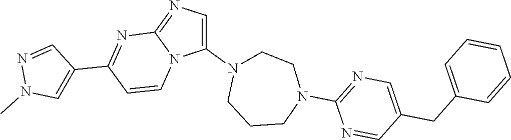 [SMILES: Cn1cc(-c2ccn3c(N4CCCN(c5ncc(Cc6ccccc6)cn5)CC4)cnc3n2)cn1] |
| 123 | 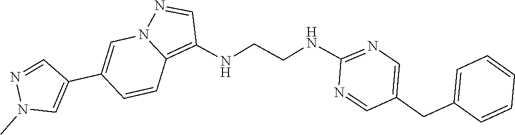 [SMILES: Cn1cc(-c2ccc3c(NCCNc4ncc(Cc5ccccc5)cn4)cnn3c2)cn1] |
| 124 | 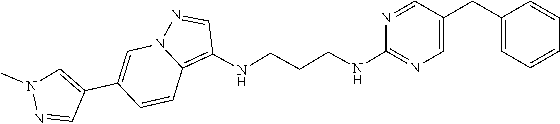 [SMILES: Cn1cc(-c2ccc3c(NCCCNc4ncc(Cc5ccccc5)cn4)cnn3c2)cn1] |
| 125 | 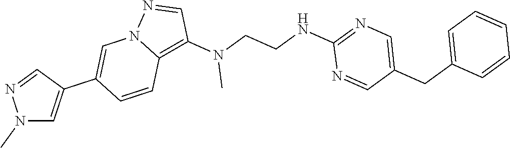 [SMILES: CN(CCNc1ncc(Cc2ccccc2)cn1)c1cnn2cc(-c3cnn(C)c3)ccc12] |
| 126 | 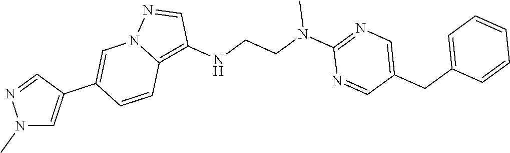 [SMILES: CN(CCNc1cnn2cc(-c3cnn(C)c3)ccc12)c1ncc(Cc2ccccc2)cn1] |
| 127 | 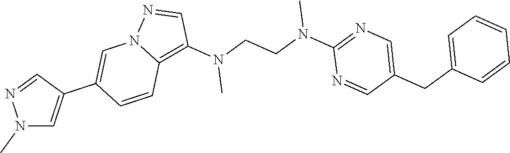 [SMILES: CN(CCN(C)c1cnn2cc(-c3cnn(C)c3)ccc12)c1ncc(Cc2ccccc2)cn1] |
| 128 | 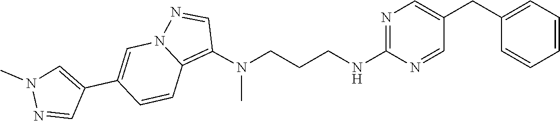 [SMILES: CN(CCCNc1ncc(Cc2ccccc2)cn1)c1cnn2cc(-c3cnn(C)c3)ccc12] |
| 129 | 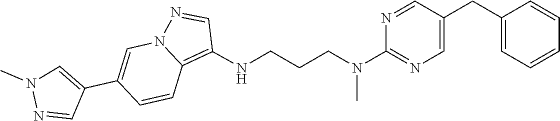 [SMILES: CN(CCCNc1cnn2cc(-c3cnn(C)c3)ccc12)c1ncc(Cc2ccccc2)cn1] |
| 130 | 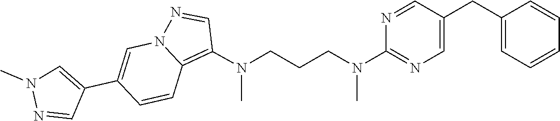 [SMILES: CN(CCCN(C)c1cnn2cc(-c3cnn(C)c3)ccc12)c1ncc(Cc2ccccc2)cn1] |
| 131 | 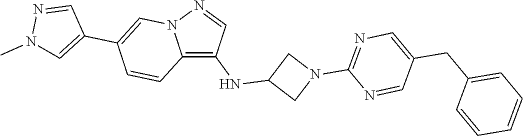 [SMILES: Cn1cc(-c2ccc3c(NC4CN(c5ncc(Cc6ccccc6)cn5)C4)cnn3c2)cn1] |
| 132 | 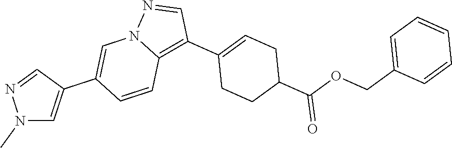 [SMILES: Cn1cc(-c2ccc3c(C4=CCC(C(=O)OCc5ccccc5)CC4)cnn3c2)cn1] |
| 133 | 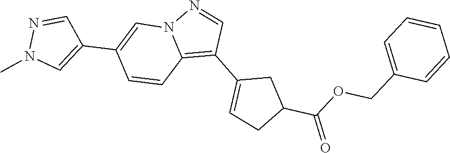 [SMILES: Cn1cc(-c2ccc3c(C4=CCC(C(=O)OCc5ccccc5)C4)cnn3c2)cn1] |
| 134 | 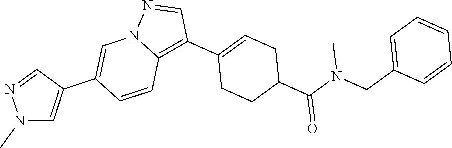 [SMILES: CN(Cc1ccccc1)C(=O)C1CC=C(c2cnn3cc(-c4cnn(C)c4)ccc23)CC1] |
| 135 | 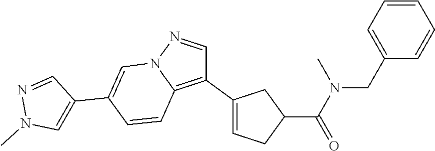 [SMILES: CN(Cc1ccccc1)C(=O)C1CC=C(c2cnn3cc(-c4cnn(C)c4)ccc23)C1] |
| 136 | 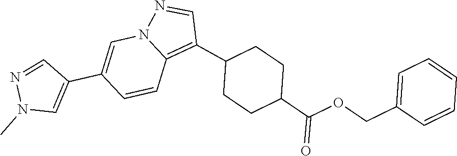 [SMILES: Cn1cc(-c2ccc3c(C4CCC(C(=O)OCc5ccccc5)CC4)cnn3c2)cn1] |
| 137 | 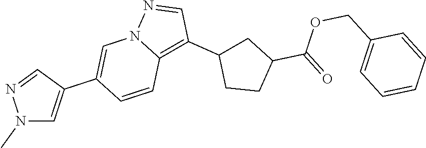 [SMILES: Cn1cc(-c2ccc3c(C4CCC(C(=O)OCc5ccccc5)C4)cnn3c2)cn1] |
| 138 | 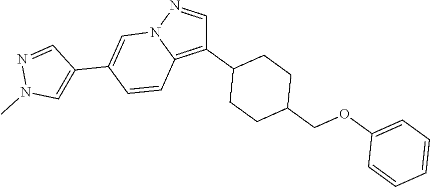 [SMILES: Cn1cc(-c2ccc3c(C4CCC(COc5ccccc5)CC4)cnn3c2)cn1] |
| 139 | 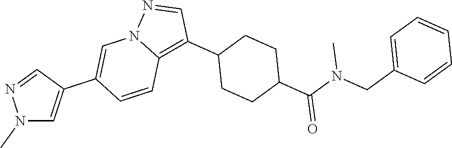 [SMILES: CN(Cc1ccccc1)C(=O)C1CCC(c2cnn3cc(-c4cnn(C)c4)ccc23)CC1] |
| 140 | 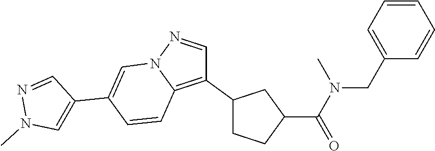 [SMILES: CN(Cc1ccccc1)C(=O)C1CCC(c2cnn3cc(-c4cnn(C)c4)ccc23)C1] |
| 141 | 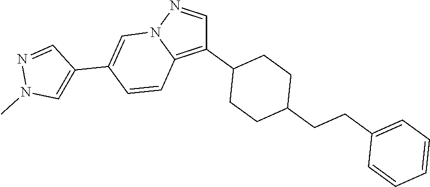 [SMILES: Cn1cc(-c2ccc3c(C4CCC(CCc5ccccc5)CC4)cnn3c2)cn1] |
| 142 | 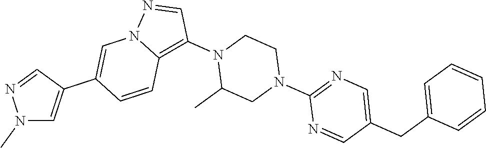 [SMILES: CC1CN(c2ncc(Cc3ccccc3)cn2)CCN1c1cnn2cc(-c3cnn(C)c3)ccc12] |
| 143 | 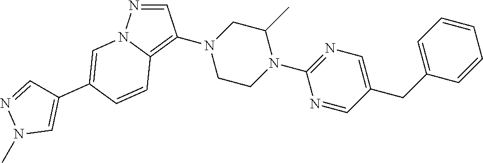 [SMILES: CC1CN(c2cnn3cc(-c4cnn(C)c4)ccc23)CCN1c1ncc(Cc2ccccc2)cn1] |
| 144 | 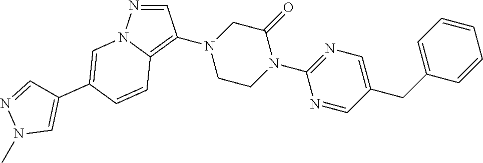 [SMILES: Cn1cc(-c2ccc3c(N4CCN(c5ncc(Cc6ccccc6)cn5)C(=O)C4)cnn3c2)cn1] |
| 145 | 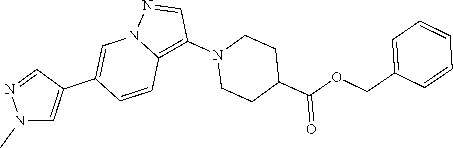 [SMILES: Cn1cc(-c2ccc3c(N4CCC(C(=O)OCc5ccccc5)CC4)cnn3c2)cn1] |
| 146 | 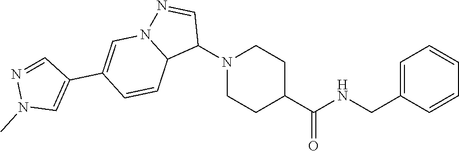 [SMILES: Cn1cc(C2=CN3N=CC(N4CCC(C(=O)NCc5ccccc5)CC4)C3C=C2)cn1] |
| 147 | 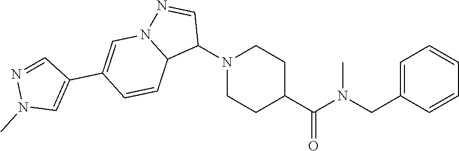 [SMILES: CN(Cc1ccccc1)C(=O)C1CCN(C2C=NN3C=C(c4cnn(C)c4)C=CC23)CC1] |
| 148 | 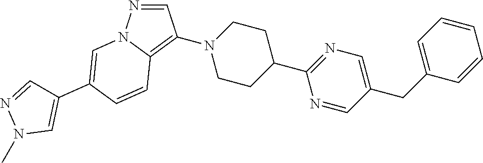 [SMILES: Cn1cc(-c2ccc3c(N4CCC(c5ncc(Cc6ccccc6)cn5)CC4)cnn3c2)cn1] |
| 149 | 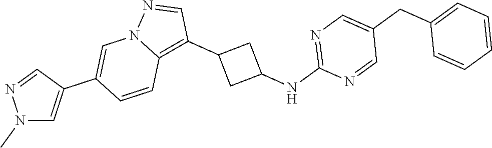 [SMILES: Cn1cc(-c2ccc3c(C4CC(Nc5ncc(Cc6ccccc6)cn5)C4)cnn3c2)cn1] |
| 150 | 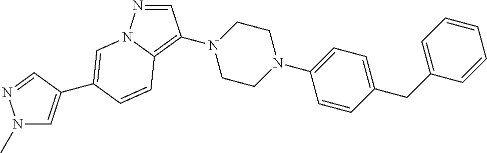 [SMILES: Cn1cc(-c2ccc3c(N4CCN(c5ccc(Cc6ccccc6)cc5)CC4)cnn3c2)cn1] |
| 151 | 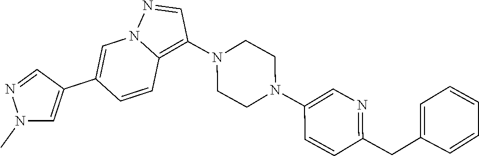 [SMILES: Cn1cc(-c2ccc3c(N4CCN(c5ccc(Cc6ccccc6)nc5)CC4)cnn3c2)cn1] |
| 152 | 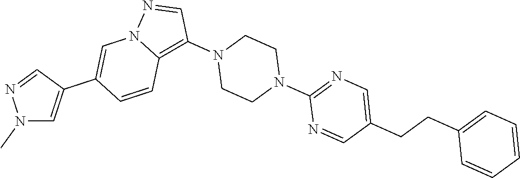 [SMILES: Cn1cc(-c2ccc3c(N4CCN(c5ncc(CCc6ccccc6)cn5)CC4)cnn3c2)cn1] |
| 153 | 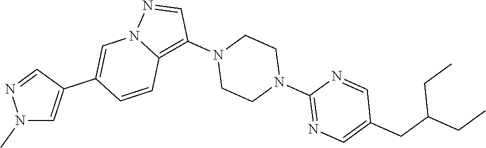 [SMILES: CCC(CC)Cc1cnc(N2CCN(c3cnn4cc(-c5cnn(C)c5)ccc34)CC2)nc1] |
| 154 | 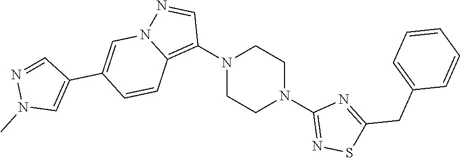 [SMILES: Cn1cc(-c2ccc3c(N4CCN(c5nsc(Cc6ccccc6)n5)CC4)cnn3c2)cn1] |
| 155 | 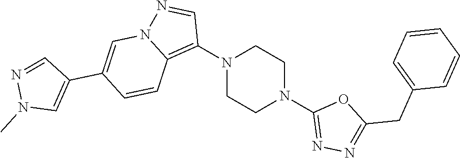 [SMILES: Cn1cc(-c2ccc3c(N4CCN(c5nnc(Cc6ccccc6)o5)CC4)cnn3c2)cn1] |
| 156 | 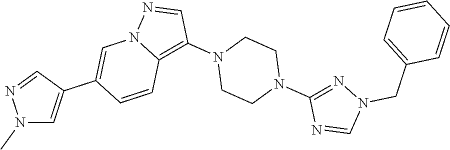 [SMILES: Cn1cc(-c2ccc3c(N4CCN(c5ncn(Cc6ccccc6)n5)CC4)cnn3c2)cn1] |
| 157 | 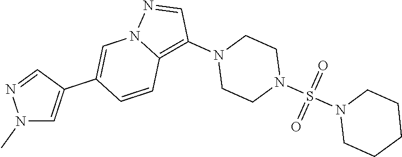 [SMILES: Cn1cc(-c2ccc3c(N4CCN(S(=O)(=O)N5CCCCC5)CC4)cnn3c2)cn1] |
| 158 | 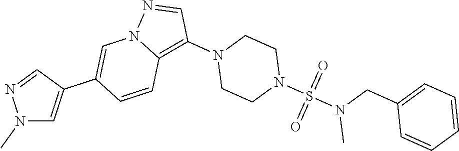 [SMILES: CN(Cc1ccccc1)S(=O)(=O)N1CCN(c2cnn3cc(-c4cnn(C)c4)ccc23)CC1] |
| 159 | 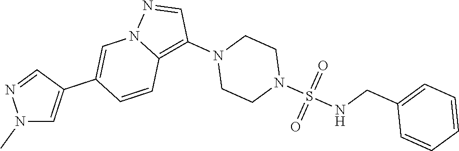 [SMILES: Cn1cc(-c2ccc3c(N4CCN(S(=O)(=O)NCc5ccccc5)CC4)cnn3c2)cn1] |
| 160 | 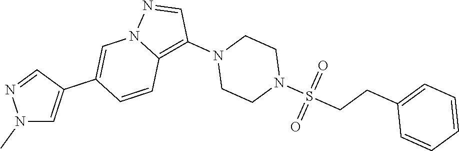 [SMILES: Cn1cc(-c2ccc3c(N4CCN(S(=O)(=O)CCc5ccccc5)CC4)cnn3c2)cn1] |
| 161 | 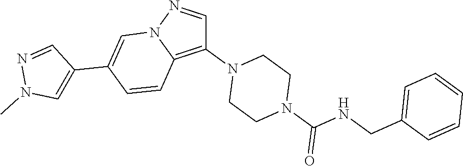 [SMILES: Cn1cc(-c2ccc3c(N4CCN(C(=O)NCc5ccccc5)CC4)cnn3c2)cn1] |
| 162 | 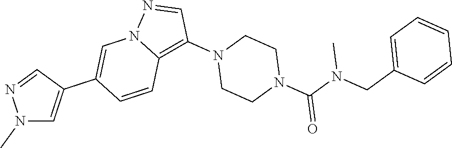 [SMILES: CN(Cc1ccccc1)C(=O)N1CCN(c2cnn3cc(-c4cnn(C)c4)ccc23)CC1] |
| 163 | 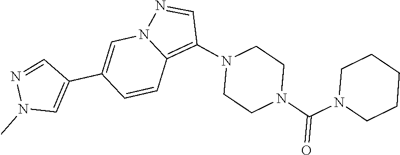 [SMILES: Cn1cc(-c2ccc3c(N4CCN(C(=O)N5CCCCC5)CC4)cnn3c2)cn1] |
| 164 | 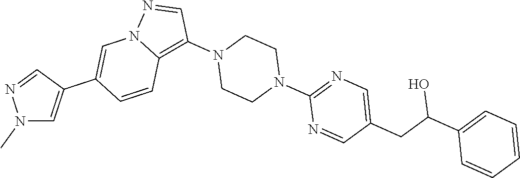 [SMILES: Cn1cc(-c2ccc3c(N4CCN(c5ncc(CC(O)c6ccccc6)cn5)CC4)cnn3c2)cn1] |
| 165 | 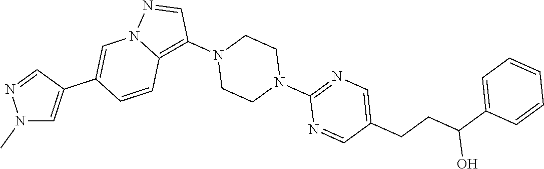 [SMILES: Cn1cc(-c2ccc3c(N4CCN(c5ncc(CCC(O)c6ccccc6)cn5)CC4)cnn3c2)cn1] |
| 166 | 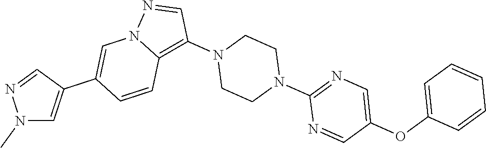 [SMILES: Cn1cc(-c2ccc3c(N4CCN(c5ncc(Oc6ccccc6)cn5)CC4)cnn3c2)cn1] |
| 167 | 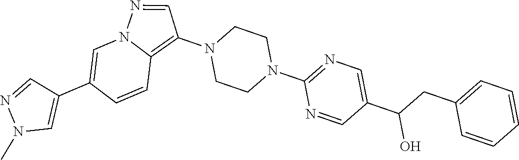 [SMILES: Cn1cc(-c2ccc3c(N4CCN(c5ncc(C(O)Cc6ccccc6)cn5)CC4)cnn3c2)cn1] |
| 168 | 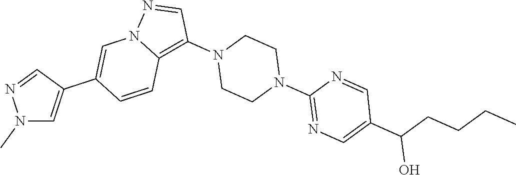 [SMILES: CCCCC(O)c1cnc(N2CCN(c3cnn4cc(-c5cnn(C)c5)ccc34)CC2)nc1] |
| 169 | 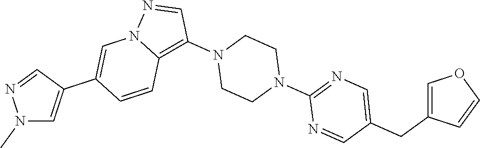 [SMILES: Cn1cc(-c2ccc3c(N4CCN(c5ncc(Cc6ccoc6)cn5)CC4)cnn3c2)cn1] |
| 170 | 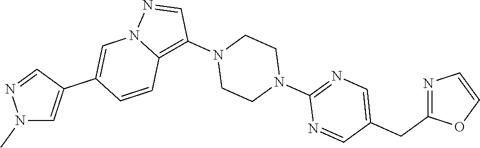 [SMILES: Cn1cc(-c2ccc3c(N4CCN(c5ncc(Cc6ncco6)cn5)CC4)cnn3c2)cn1] |
| 171 | 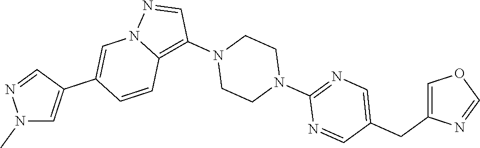 [SMILES: Cn1cc(-c2ccc3c(N4CCN(c5ncc(Cc6cocn6)cn5)CC4)cnn3c2)cn1] |
| 172 | 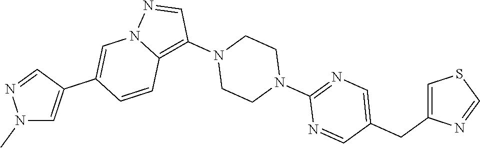 [SMILES: Cn1cc(-c2ccc3c(N4CCN(c5ncc(Cc6cscn6)cn5)CC4)cnn3c2)cn1] |
| 173 | 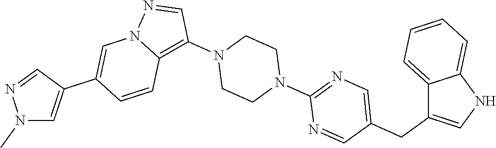 [SMILES: Cn1cc(-c2ccc3c(N4CCN(c5ncc(Cc6c[nH]c7ccccc67)cn5)CC4)cnn3c2)cn1] |
| 174 | 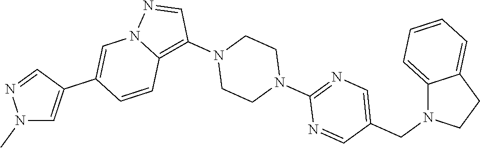 [SMILES: Cn1cc(-c2ccc3c(N4CCN(c5ncc(CN6CCc7ccccc76)cn5)CC4)cnn3c2)cn1] |
| 175 | 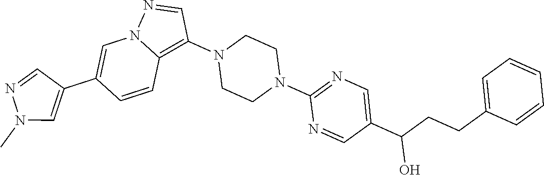 [SMILES: Cn1cc(-c2ccc3c(N4CCN(c5ncc(C(O)CCc6ccccc6)cn5)CC4)cnn3c2)cn1] |
| 176 | 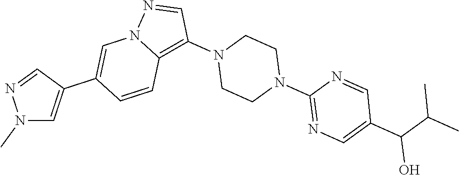 [SMILES: CC(C)C(O)c1cnc(N2CCN(c3cnn4cc(-c5cnn(C)c5)ccc34)CC2)nc1] |
| 177 | 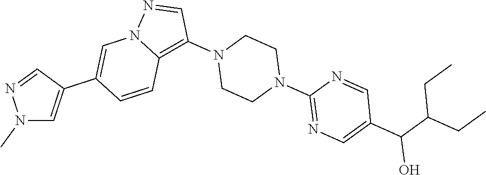 [SMILES: CCC(CC)C(O)c1cnc(N2CCN(c3cnn4cc(-c5cnn(C)c5)ccc34)CC2)nc1] |
| 178 | 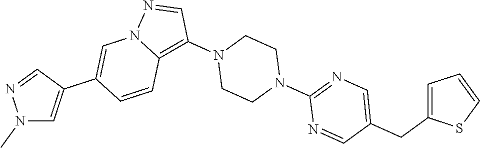 [SMILES: Cn1cc(-c2ccc3c(N4CCN(c5ncc(Cc6cccs6)cn5)CC4)cnn3c2)cn1] |
| 179 | 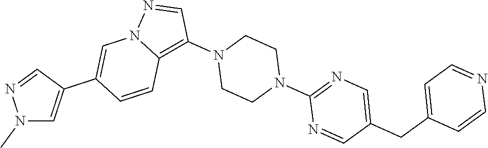 [SMILES: Cn1cc(-c2ccc3c(N4CCN(c5ncc(Cc6ccncc6)cn5)CC4)cnn3c2)cn1] |
| 180 | 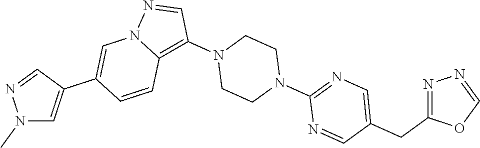 [SMILES: Cn1cc(-c2ccc3c(N4CCN(c5ncc(Cc6nnco6)cn5)CC4)cnn3c2)cn1] |
| 181 | 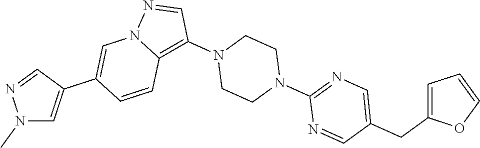 [SMILES: Cn1cc(-c2ccc3c(N4CCN(c5ncc(Cc6ccco6)cn5)CC4)cnn3c2)cn1] |
| 182 | 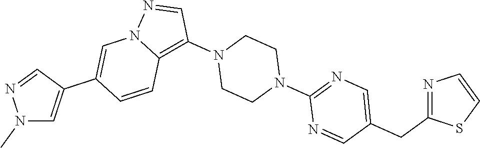 [SMILES: Cn1cc(-c2ccc3c(N4CCN(c5ncc(Cc6nccs6)cn5)CC4)cnn3c2)cn1] |
| 183 | 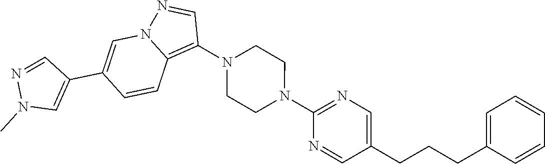 [SMILES: Cn1cc(-c2ccc3c(N4CCN(c5ncc(CCCc6ccccc6)cn5)CC4)cnn3c2)cn1] |
| 184 | 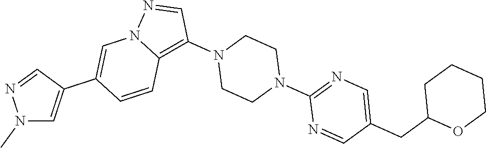 [SMILES: Cn1cc(-c2ccc3c(N4CCN(c5ncc(CC6CCCCO6)cn5)CC4)cnn3c2)cn1] |
| 185 | 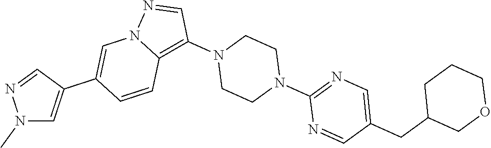 [SMILES: Cn1cc(-c2ccc3c(N4CCN(c5ncc(CC6CCCOC6)cn5)CC4)cnn3c2)cn1] |
| 186 | 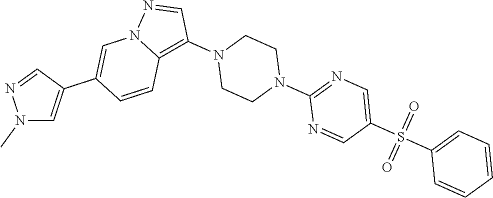 [SMILES: Cn1cc(-c2ccc3c(N4CCN(c5ncc(S(=O)(=O)c6ccccc6)cn5)CC4)cnn3c2)cn1] |
| 187 | 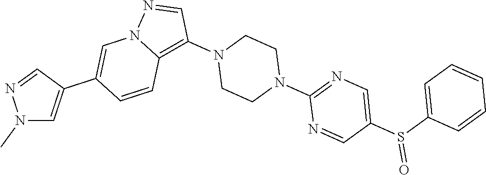 [SMILES: Cn1cc(-c2ccc3c(N4CCN(c5ncc(S(=O)c6ccccc6)cn5)CC4)cnn3c2)cn1] |
| 188 | 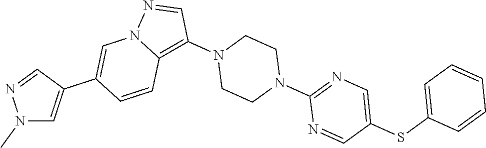 [SMILES: Cn1cc(-c2ccc3c(N4CCN(c5ncc(Sc6ccccc6)cn5)CC4)cnn3c2)cn1] |
| 189 | 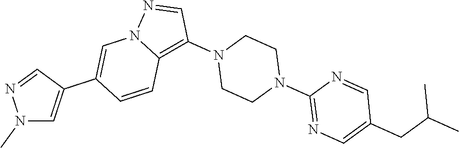 [SMILES: CC(C)Cc1cnc(N2CCN(c3cnn4cc(-c5cnn(C)c5)ccc34)CC2)nc1] |
| 190 | 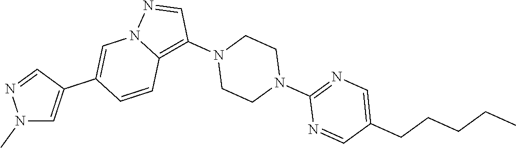 [SMILES: CCCCCc1cnc(N2CCN(c3cnn4cc(-c5cnn(C)c5)ccc34)CC2)nc1] |
| 191 | 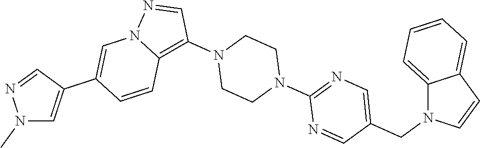 [SMILES: Cn1cc(-c2ccc3c(N4CCN(c5ncc(Cn6ccc7ccccc76)cn5)CC4)cnn3c2)cn1] |
| 192 | 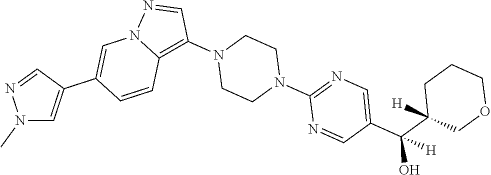 |
| 193 | 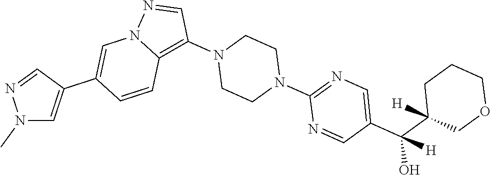 |
| 194 | 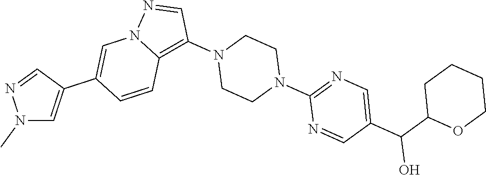 [SMILES: Cn1cc(-c2ccc3c(N4CCN(c5ncc(C(O)C6CCCCO6)cn5)CC4)cnn3c2)cn1] |
| 195 | 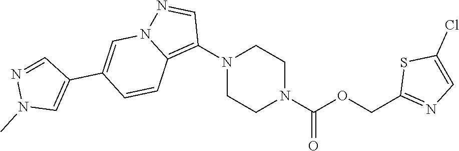 [SMILES: Cn1cc(-c2ccc3c(N4CCN(C(=O)OCc5ncc(Cl)s5)CC4)cnn3c2)cn1] |
| 196 | 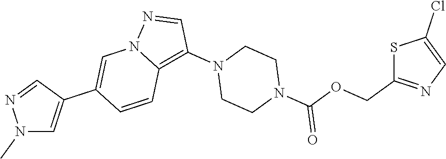 [SMILES: Cn1cc(-c2ccc3c(N4CCN(C(=O)OCc5ncc(Cl)s5)CC4)cnn3c2)cn1] |
| 197 | 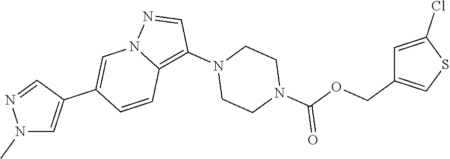 [SMILES: Cn1cc(-c2ccc3c(N4CCN(C(=O)OCc5csc(Cl)c5)CC4)cnn3c2)cn1] |
| 198 | 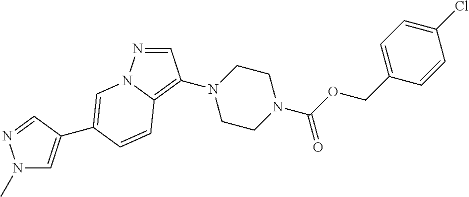 [SMILES: Cn1cc(-c2ccc3c(N4CCN(C(=O)OCc5ccc(Cl)cc5)CC4)cnn3c2)cn1] |
| 199 | 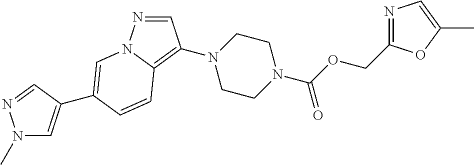 |
| 200 | 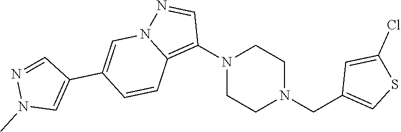 [SMILES: Cn1cc(-c2ccc3c(N4CCN(Cc5csc(Cl)c5)CC4)cnn3c2)cn1] |
| 201 | 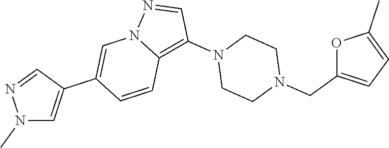 [SMILES: Cc1ccc(CN2CCN(c3cnn4cc(-c5cnn(C)c5)ccc34)CC2)o1] |
| 202 | 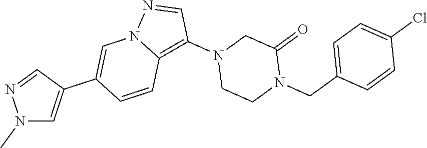 [SMILES: Cn1cc(-c2ccc3c(N4CCN(Cc5ccc(Cl)cc5)C(=O)C4)cnn3c2)cn1] |
| 203 | 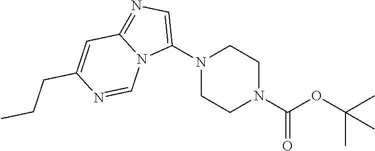 [SMILES: CCCc1cc2ncc(N3CCN(C(=O)OC(C)(C)C)CC3)n2cn1] |
| 204 | 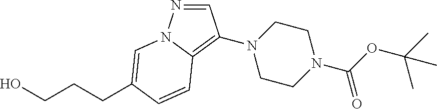 [SMILES: CC(C)(C)OC(=O)N1CCN(c2cnn3cc(CCCO)ccc23)CC1] |
| 205 | 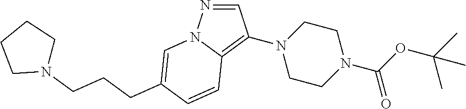 [SMILES: CC(C)(C)OC(=O)N1CCN(c2cnn3cc(CCCN4CCCC4)ccc23)CC1] |
| 206 | 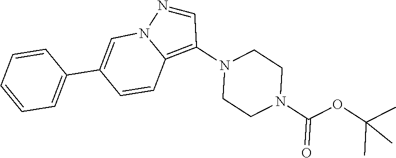 [SMILES: CC(C)(C)OC(=O)N1CCN(c2cnn3cc(-c4ccccc4)ccc23)CC1] |
| 207 | 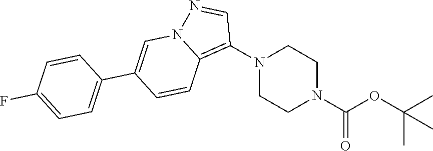 [SMILES: CC(C)(C)OC(=O)N1CCN(c2cnn3cc(-c4ccc(F)cc4)ccc23)CC1] |
| 208 | 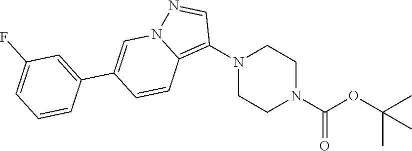 [SMILES: CC(C)(C)OC(=O)N1CCN(c2cnn3cc(-c4cccc(F)c4)ccc23)CC1] |
| 209 | 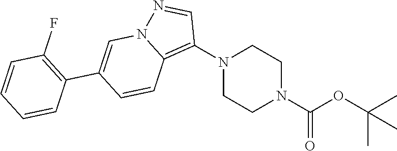 [SMILES: CC(C)(C)OC(=O)N1CCN(c2cnn3cc(-c4ccccc4F)ccc23)CC1] |
| 210 | 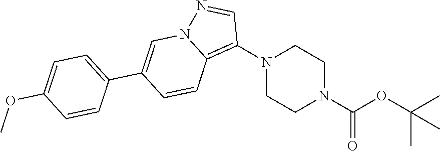 [SMILES: COc1ccc(-c2ccc3c(N4CCN(C(=O)OC(C)(C)C)CC4)cnn3c2)cc1] |
| 211 | 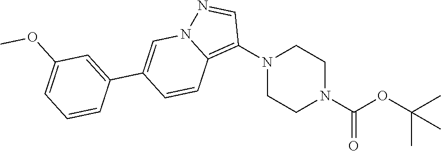 [SMILES: COc1cccc(-c2ccc3c(N4CCN(C(=O)OC(C)(C)C)CC4)cnn3c2)c1] |
| 212 | 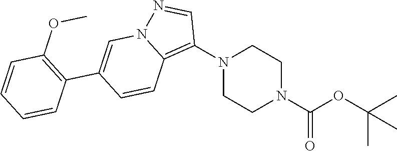 [SMILES: COc1ccccc1-c1ccc2c(N3CCN(C(=O)OC(C)(C)C)CC3)cnn2c1] |
| 213 | 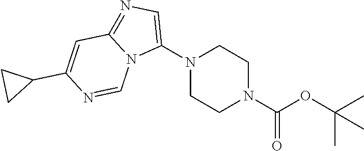 [SMILES: CC(C)(C)OC(=O)N1CCN(c2cnc3cc(C4CC4)ncn23)CC1] |
| 214 | 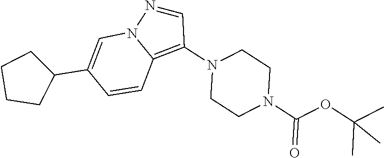 [SMILES: CC(C)(C)OC(=O)N1CCN(c2cnn3cc(C4CCCC4)ccc23)CC1] |
| 215 | 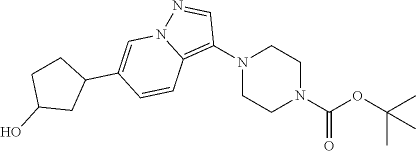 [SMILES: CC(C)(C)OC(=O)N1CCN(c2cnn3cc(C4CCC(O)C4)ccc23)CC1] |
| 216 | 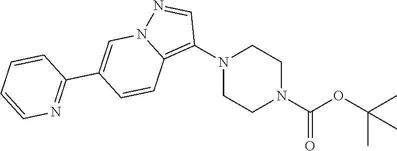 [SMILES: CC(C)(C)OC(=O)N1CCN(c2cnn3cc(-c4ccccn4)ccc23)CC1] |
| 217 | 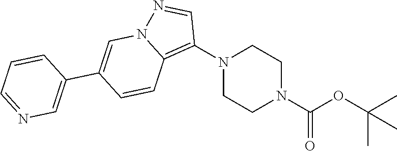 [SMILES: CC(C)(C)OC(=O)N1CCN(c2cnn3cc(-c4cccnc4)ccc23)CC1] |
| 218 | 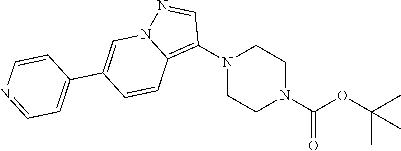 [SMILES: CC(C)(C)OC(=O)N1CCN(c2cnn3cc(-c4ccncc4)ccc23)CC1] |
| 219 | 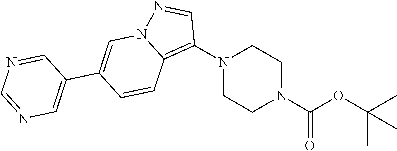 [SMILES: CC(C)(C)OC(=O)N1CCN(c2cnn3cc(-c4cncnc4)ccc23)CC1] |
| 220 | 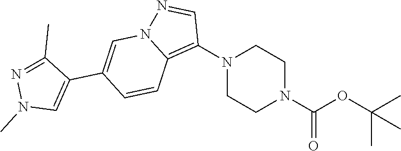 [SMILES: Cc1nn(C)cc1-c1ccc2c(N3CCN(C(=O)OC(C)(C)C)CC3)cnn2c1] |
| 221 | 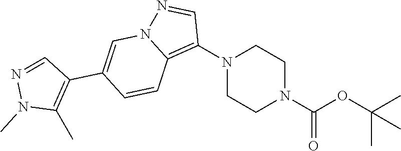 [SMILES: Cc1c(-c2ccc3c(N4CCN(C(=O)OC(C)(C)C)CC4)cnn3c2)cnn1C] |
| 222 | 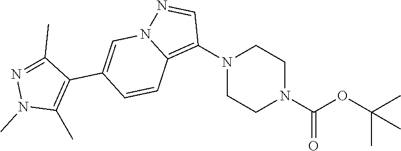 [SMILES: Cc1nn(C)c(C)c1-c1ccc2c(N3CCN(C(=O)OC(C)(C)C)CC3)cnn2c1] |
| 223 | 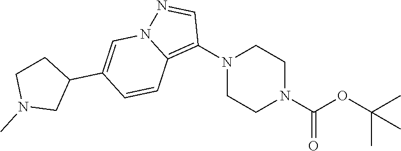 [SMILES: CN1CCC(c2ccc3c(N4CCN(C(=O)OC(C)(C)C)CC4)cnn3c2)C1] |
| 224 | 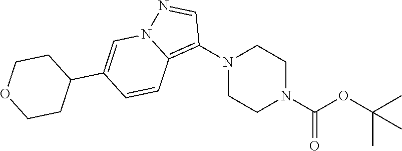 [SMILES: CC(C)(C)OC(=O)N1CCN(c2cnn3cc(C4CCOCC4)ccc23)CC1] |
| 225 | 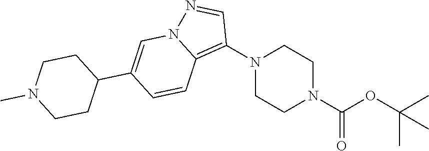 [SMILES: CN1CCC(c2ccc3c(N4CCN(C(=O)OC(C)(C)C)CC4)cnn3c2)CC1] |
| 226 | 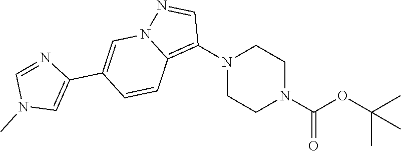 [SMILES: Cn1cnc(-c2ccc3c(N4CCN(C(=O)OC(C)(C)C)CC4)cnn3c2)c1] |
| 227 | 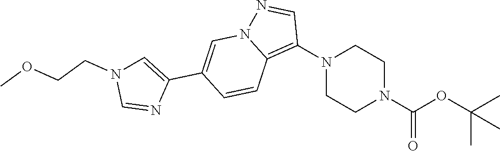 [SMILES: COCCn1cnc(-c2ccc3c(N4CCN(C(=O)OC(C)(C)C)CC4)cnn3c2)c1] |
| 228 | 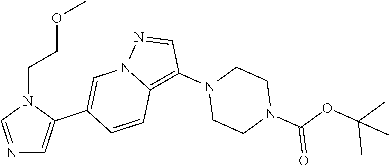 [SMILES: COCCn1cncc1-c1ccc2c(N3CCN(C(=O)OC(C)(C)C)CC3)cnn2c1] |
| 229 | 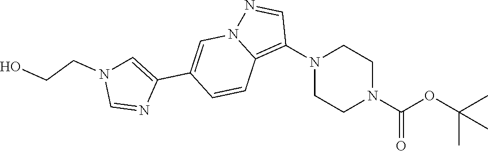 [SMILES: CC(C)(C)OC(=O)N1CCN(c2cnn3cc(-c4cn(CCO)cn4)ccc23)CC1] |
| 230 | 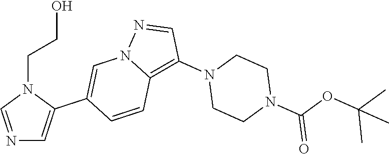 [SMILES: CC(C)(C)OC(=O)N1CCN(c2cnn3cc(-c4cncn4CCO)ccc23)CC1] |
| 231 | 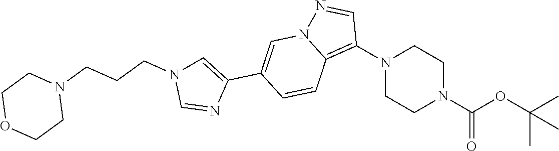 [SMILES: CC(C)(C)OC(=O)N1CCN(c2cnn3cc(-c4cn(CCCN5CCOCC5)cn4)ccc23)CC1] |
| 232 | 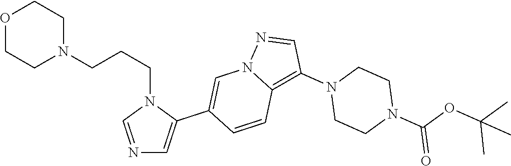 [SMILES: CC(C)(C)OC(=O)N1CCN(c2cnn3cc(-c4cncn4CCCN4CCOCC4)ccc23)CC1] |
| 233 | 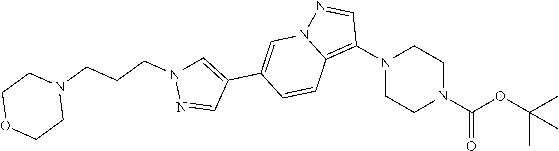 [SMILES: CC(C)(C)OC(=O)N1CCN(c2cnn3cc(-c4cnn(CCCN5CCOCC5)c4)ccc23)CC1] |
| 234 | 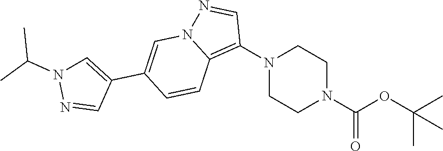 [SMILES: CC(C)n1cc(-c2ccc3c(N4CCN(C(=O)OC(C)(C)C)CC4)cnn3c2)cn1] |
| 235 | 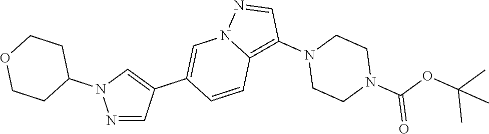 [SMILES: CC(C)(C)OC(=O)N1CCN(c2cnn3cc(-c4cnn(C5CCOCC5)c4)ccc23)CC1] |
| 236 | 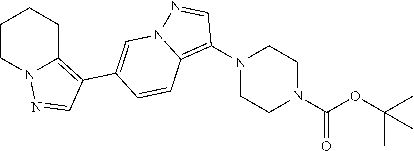 [SMILES: CC(C)(C)OC(=O)N1CCN(c2cnn3cc(-c4cnn5c4CCCC5)ccc23)CC1] |
| 237 | 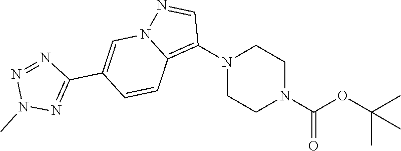 [SMILES: Cn1nnc(-c2ccc3c(N4CCN(C(=O)OC(C)(C)C)CC4)cnn3c2)n1] |
| 238 | 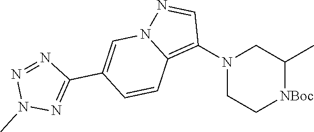 |
| 239 | 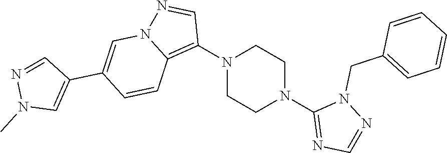 [SMILES: Cn1cc(-c2ccc3c(N4CCN(c5ncnn5Cc5ccccc5)CC4)cnn3c2)cn1] |
| 240 | 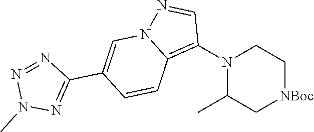 [SMILES: CC1CN(C(=O)OC(C)(C)C)CCN1c1cnn2cc(-c3nnn(C)n3)ccc12] |
| 241 | 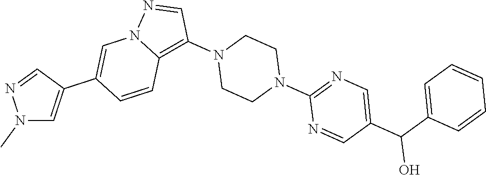 [SMILES: Cn1cc(-c2ccc3c(N4CCN(c5ncc(C(O)c6ccccc6)cn5)CC4)cnn3c2)cn1] |
| 242 | 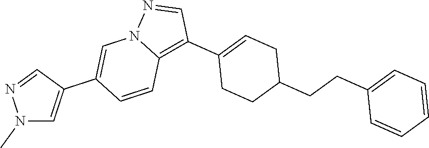 [SMILES: Cn1cc(-c2ccc3c(C4=CCC(CCc5ccccc5)CC4)cnn3c2)cn1] |
| 243 | 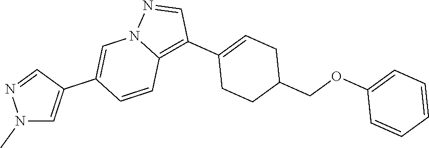 [SMILES: Cn1cc(-c2ccc3c(C4=CCC(COc5ccccc5)CC4)cnn3c2)cn1] |
| 244 | 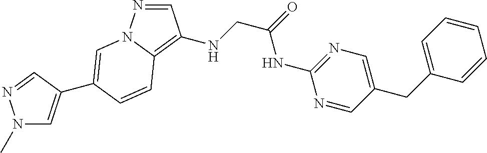 [SMILES: Cn1cc(-c2ccc3c(NCC(=O)Nc4ncc(Cc5ccccc5)cn4)cnn3c2)cn1] |
| 245 | 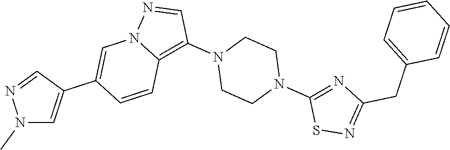 [SMILES: Cn1cc(-c2ccc3c(N4CCN(c5nc(Cc6ccccc6)ns5)CC4)cnn3c2)cn1] |
| 246 | 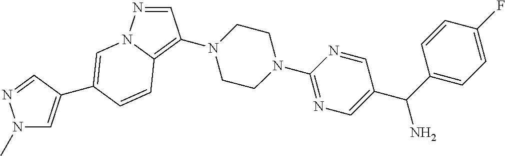 [SMILES: Cn1cc(-c2ccc3c(N4CCN(c5ncc(C(N)c6ccc(F)cc6)cn5)CC4)cnn3c2)cn1] |
| 247 | 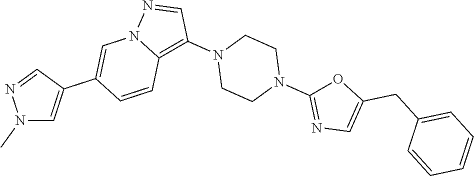 [SMILES: Cn1cc(-c2ccc3c(N4CCN(c5ncc(Cc6ccccc6)o5)CC4)cnn3c2)cn1] |
| 248 | 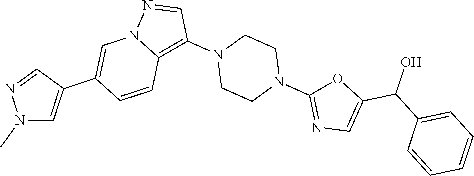 [SMILES: Cn1cc(-c2ccc3c(N4CCN(c5ncc(C(O)c6ccccc6)o5)CC4)cnn3c2)cn1] |
| 249 | 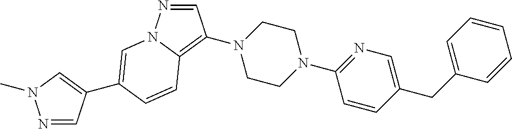 [SMILES: Cn1cc(-c2ccc3c(N4CCN(c5ccc(Cc6ccccc6)cn5)CC4)cnn3c2)cn1] |
| 250 | 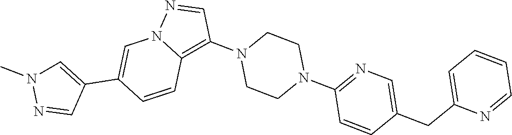 [SMILES: Cn1cc(-c2ccc3c(N4CCN(c5ccc(Cc6ccccn6)cn5)CC4)cnn3c2)cn1] |
| 251 | 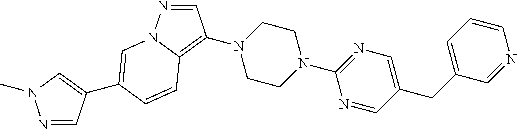 [SMILES: Cn1cc(-c2ccc3c(N4CCN(c5ncc(Cc6cccnc6)cn5)CC4)cnn3c2)cn1] |
| 252 | 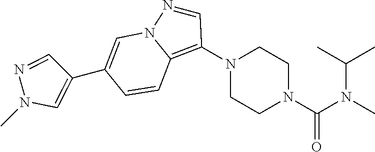 [SMILES: CC(C)N(C)C(=O)N1CCN(c2cnn3cc(-c4cnn(C)c4)ccc23)CC1] |
| 253 | 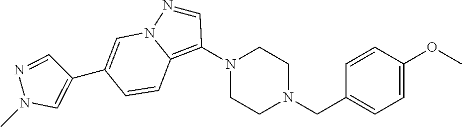 |
| 254 | 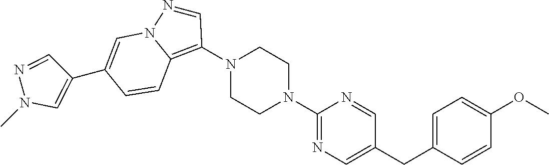 |
| 255 | 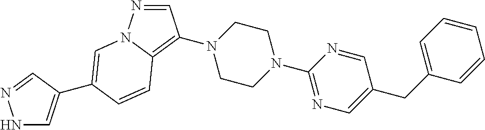 [SMILES: c1ccc(Cc2cnc(N3CCN(c4cnn5cc(-c6cn[nH]c6)ccc45)CC3)nc2)cc1] |
| 256 | 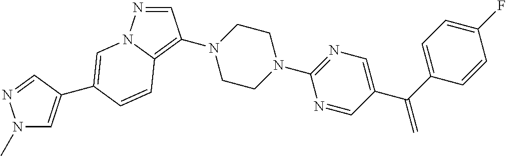 [SMILES: C=C(c1ccc(F)cc1)c1cnc(N2CCN(c3cnn4cc(-c5cnn(C)c5)ccc34)CC2)nc1] |
| 257 | 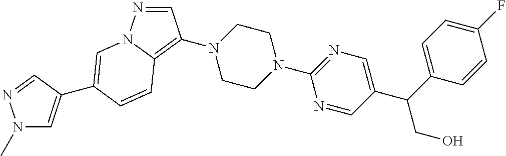 [SMILES: Cn1cc(-c2ccc3c(N4CCN(c5ncc(C(CO)c6ccc(F)cc6)cn5)CC4)cnn3c2)cn1] |
| 258 | 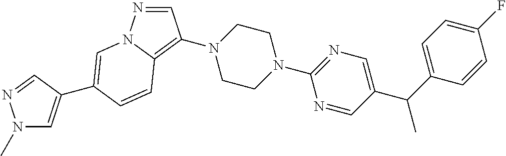 [SMILES: CC(c1ccc(F)cc1)c1cnc(N2CCN(c3cnn4cc(-c5cnn(C)c5)ccc34)CC2)nc1] |
| 259 | 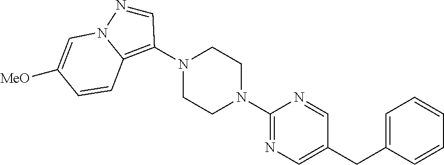 [SMILES: COc1ccc2c(N3CCN(c4ncc(Cc5ccccc5)cn4)CC3)cnn2c1] |
| 260 | 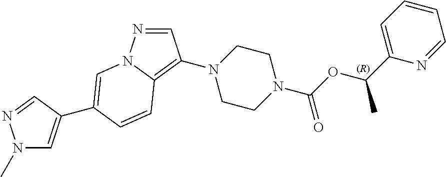 |
| 261 | 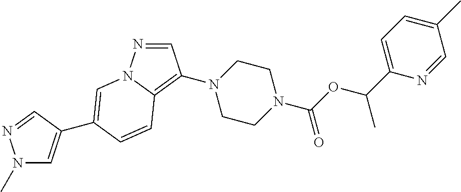 |
| 262 | 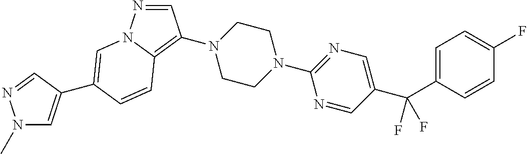 [SMILES: Cn1cc(-c2ccc3c(N4CCN(c5ncc(C(F)(F)c6ccc(F)cc6)cn5)CC4)cnn3c2)cn1] |
| 263 | 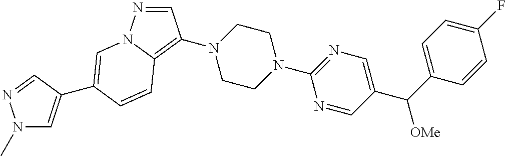 [SMILES: COC(c1ccc(F)cc1)c1cnc(N2CCN(c3cnn4cc(-c5cnn(C)c5)ccc34)CC2)nc1] |
| 264 | 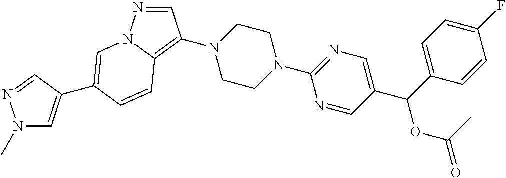 [SMILES: CC(=O)OC(c1ccc(F)cc1)c1cnc(N2CCN(c3cnn4cc(-c5cnn(C)c5)ccc34)CC2)nc1] |
| 265 | 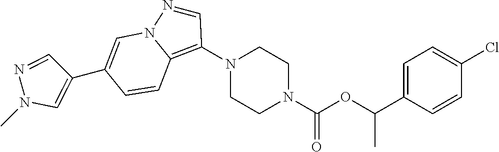 [SMILES: CC(OC(=O)N1CCN(c2cnn3cc(-c4cnn(C)c4)ccc23)CC1)c1ccc(Cl)cc1] |
| 266 | 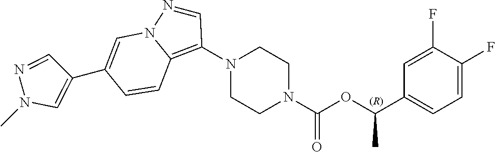 |
| 267 | 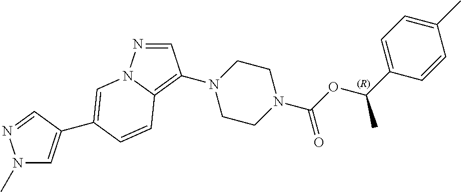 |
| 268 | 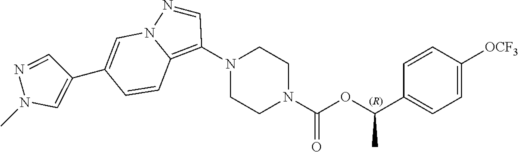 |
| 269 | 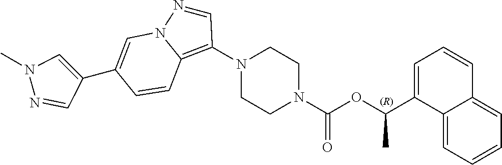 |
| 270 | 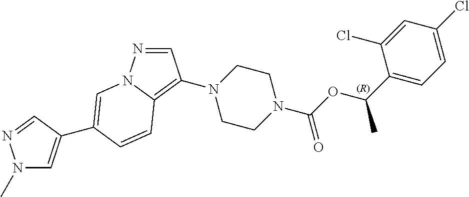 |
| 271 | 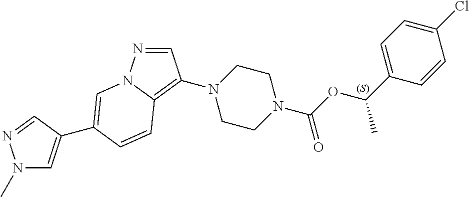 |
| 272 | 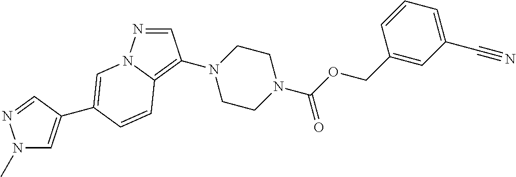 [SMILES: Cn1cc(-c2ccc3c(N4CCN(C(=O)OCc5cccc(C#N)c5)CC4)cnn3c2)cn1] |
| 273 | 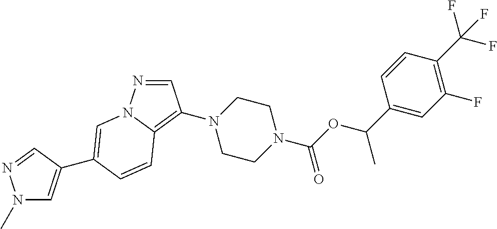 [SMILES: CC(OC(=O)N1CCN(c2cnn3cc(-c4cnn(C)c4)ccc23)CC1)c1ccc(C(F)(F)F)c(F)c1] |
| 274 | 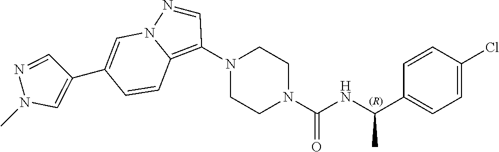 |
| 275 | 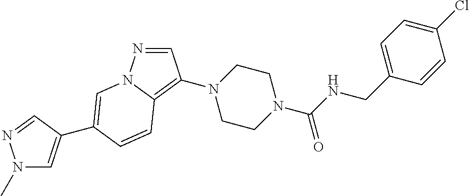 [SMILES: Cn1cc(-c2ccc3c(N4CCN(C(=O)NCc5ccc(Cl)cc5)CC4)cnn3c2)cn1] |
| 276 | 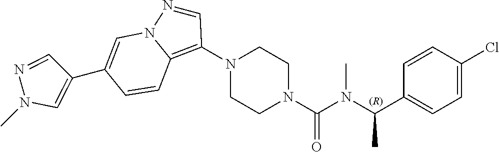 |
| 277 | 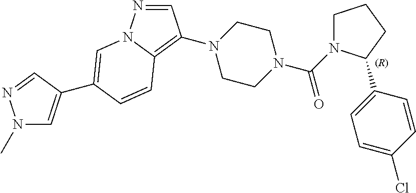 |
| 278 | 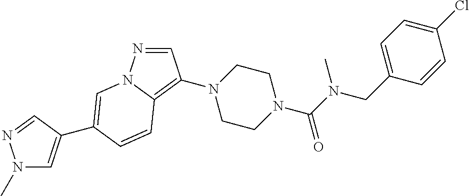 [SMILES: CN(Cc1ccc(Cl)cc1)C(=O)N1CCN(c2cnn3cc(-c4cnn(C)c4)ccc23)CC1] |
| 279 | 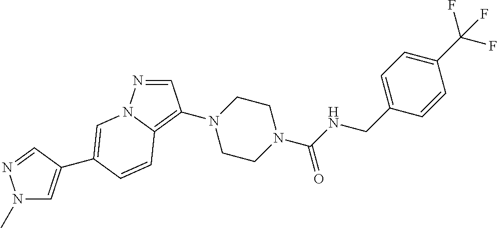 [SMILES: Cn1cc(-c2ccc3c(N4CCN(C(=O)NCc5ccc(C(F)(F)F)cc5)CC4)cnn3c2)cn1] |
| 280 | 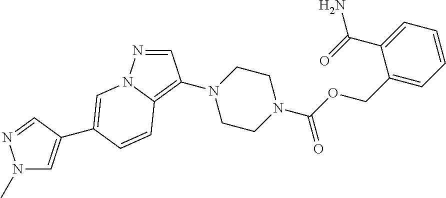 [SMILES: Cn1cc(-c2ccc3c(N4CCN(C(=O)OCc5ccccc5C(N)=O)CC4)cnn3c2)cn1] |
| 281 | 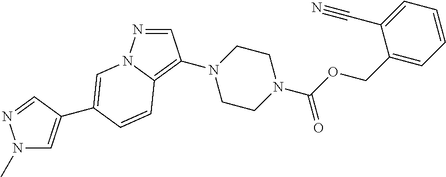 [SMILES: Cn1cc(-c2ccc3c(N4CCN(C(=O)OCc5ccccc5C#N)CC4)cnn3c2)cn1] |
| 282 | 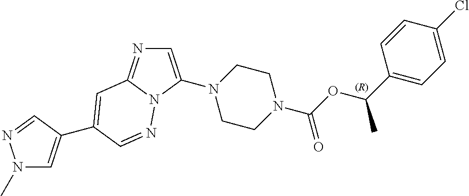 |
| 283 | 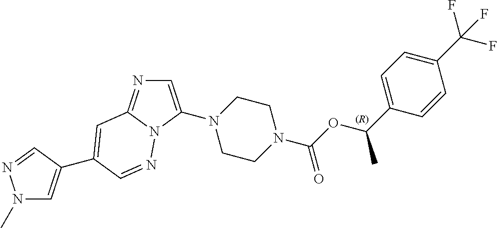 |
| 284 | 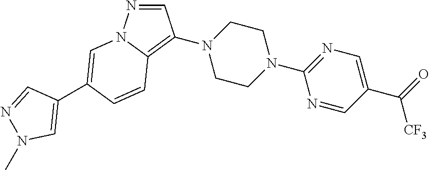 [SMILES: Cn1cc(-c2ccc3c(N4CCN(c5ncc(C(=O)C(F)(F)F)cn5)CC4)cnn3c2)cn1] |
| 285 | 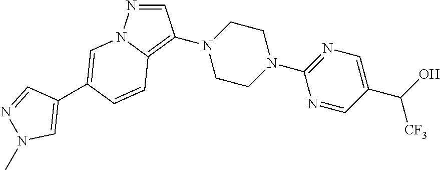 [SMILES: Cn1cc(-c2ccc3c(N4CCN(c5ncc(C(O)C(F)(F)F)cn5)CC4)cnn3c2)cn1] |
| 286 | 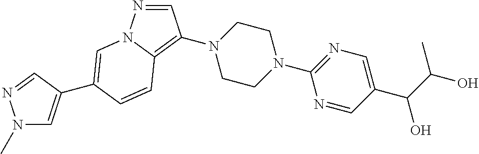 [SMILES: CC(O)C(O)c1cnc(N2CCN(c3cnn4cc(-c5cnn(C)c5)ccc34)CC2)nc1] |
| 287 | 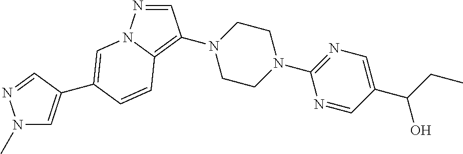 [SMILES: CCC(O)c1cnc(N2CCN(c3cnn4cc(-c5cnn(C)c5)ccc34)CC2)nc1] |
| 288 | 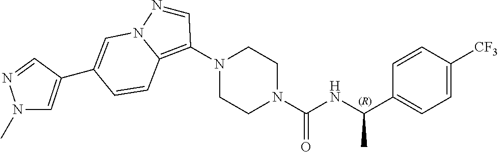 |
| 289 | 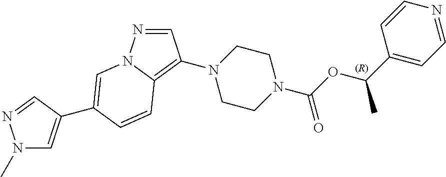 |
| 290 | 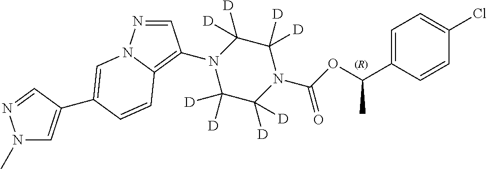 |
| 291 | 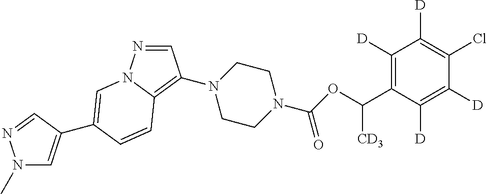 [SMILES: [2H]c1c([2H])c(C(OC(=O)N2CCN(c3cnn4cc(-c5cnn(C)c5)ccc34)CC2)C([2H])([2H])[2H])c([2H])c([2H])c1Cl] |
| 292 | 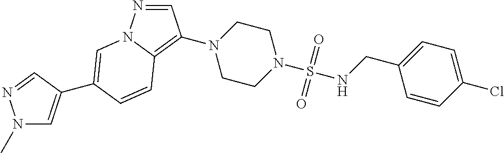 [SMILES: Cn1cc(-c2ccc3c(N4CCN(S(=O)(=O)NCc5ccc(Cl)cc5)CC4)cnn3c2)cn1] |
| 293 | 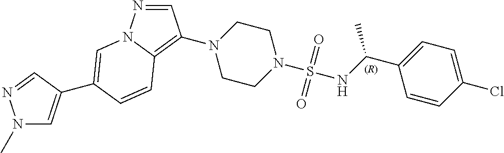 |
| 294 | 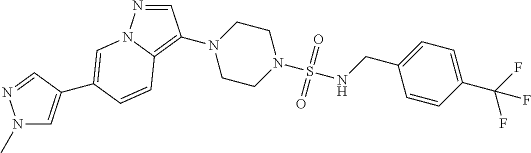 [SMILES: Cn1cc(-c2ccc3c(N4CCN(S(=O)(=O)NCc5ccc(C(F)(F)F)cc5)CC4)cnn3c2)cn1] |
| 295 | 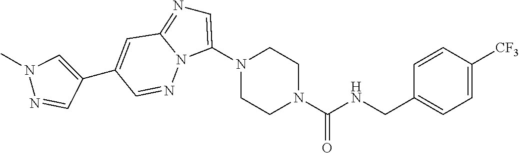 [SMILES: Cn1cc(-c2cnn3c(N4CCN(C(=O)NCc5ccc(C(F)(F)F)cc5)CC4)cnc3c2)cn1] |
| 296 | 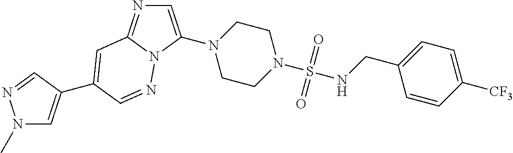 [SMILES: Cn1cc(-c2cnn3c(N4CCN(S(=O)(=O)NCc5ccc(C(F)(F)F)cc5)CC4)cnc3c2)cn1] |
| 297 | 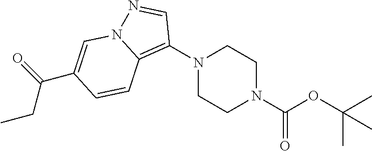 [SMILES: CCC(=O)c1ccc2c(N3CCN(C(=O)OC(C)(C)C)CC3)cnn2c1] |
| 298 | 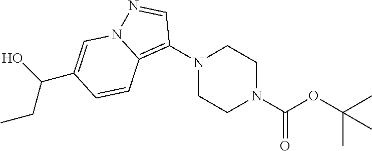 [SMILES: CCC(O)c1ccc2c(N3CCN(C(=O)OC(C)(C)C)CC3)cnn2c1] |
| 299 | 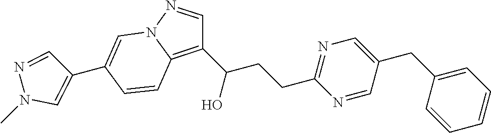 [SMILES: Cn1cc(-c2ccc3c(C(O)CCc4ncc(Cc5ccccc5)cn4)cnn3c2)cn1] |
| 300 | 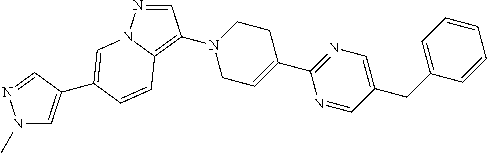 [SMILES: Cn1cc(-c2ccc3c(N4CC=C(c5ncc(Cc6ccccc6)cn5)CC4)cnn3c2)cn1] |
| 301 | 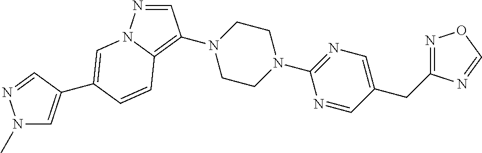 [SMILES: Cn1cc(-c2ccc3c(N4CCN(c5ncc(Cc6ncon6)cn5)CC4)cnn3c2)cn1] |
| 302 | 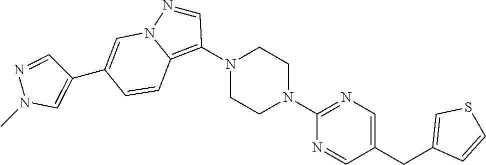 [SMILES: Cn1cc(-c2ccc3c(N4CCN(c5ncc(Cc6ccsc6)cn5)CC4)cnn3c2)cn1] |
| 303 | 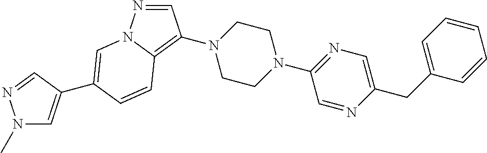 [SMILES: Cn1cc(-c2ccc3c(N4CCN(c5cnc(Cc6ccccc6)cn5)CC4)cnn3c2)cn1] |
| 304 | 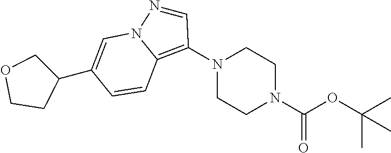 [SMILES: CC(C)(C)OC(=O)N1CCN(c2cnn3cc(C4CCOC4)ccc23)CC1] |
| 305 | 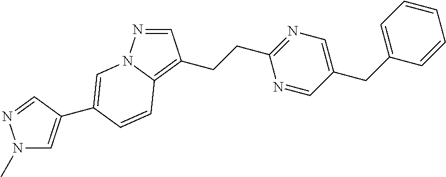 [SMILES: Cn1cc(-c2ccc3c(CCc4ncc(Cc5ccccc5)cn4)cnn3c2)cn1] |
| 306 | 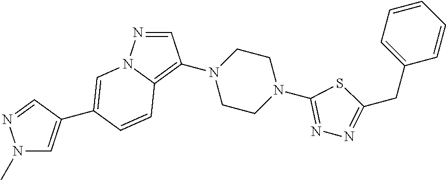 [SMILES: Cn1cc(-c2ccc3c(N4CCN(c5nnc(Cc6ccccc6)s5)CC4)cnn3c2)cn1] |
| 307 | 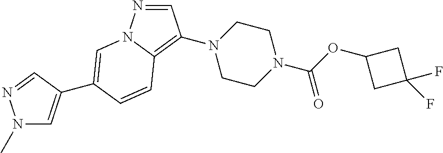 [SMILES: Cn1cc(-c2ccc3c(N4CCN(C(=O)OC5CC(F)(F)C5)CC4)cnn3c2)cn1] |
| 308 | 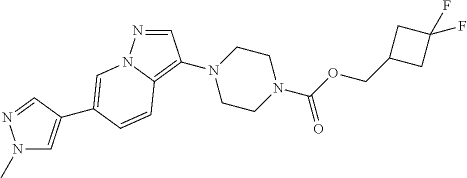 [SMILES: Cn1cc(-c2ccc3c(N4CCN(C(=O)OCC5CC(F)(F)C5)CC4)cnn3c2)cn1] |
| 309 | 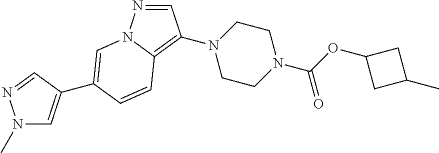 |
| 310 | 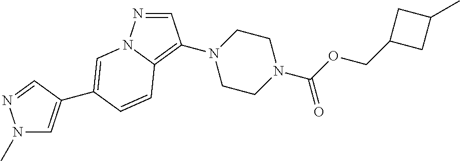 |
| 311 | 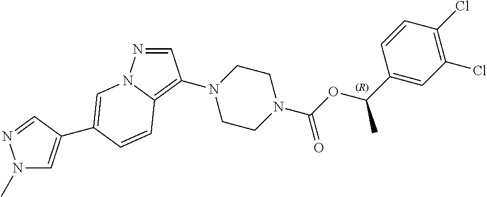 |
| 312 | 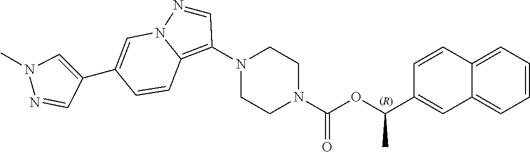 |
| 313 | 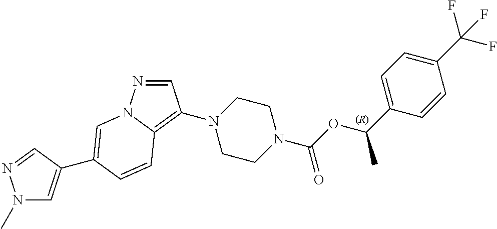 |
| 314 | 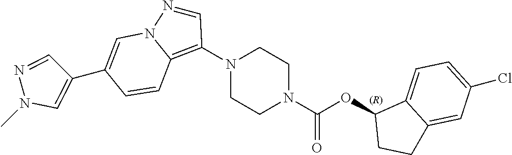 |
| 315 | 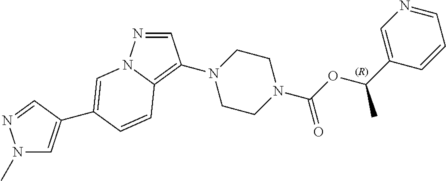 |
| 316 | 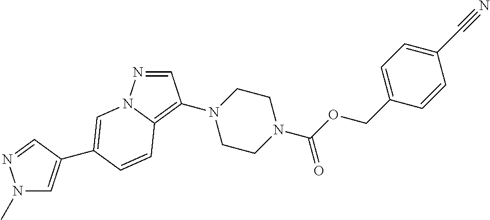 [SMILES: Cn1cc(-c2ccc3c(N4CCN(C(=O)OCc5ccc(C#N)cc5)CC4)cnn3c2)cn1] |
| 317 | 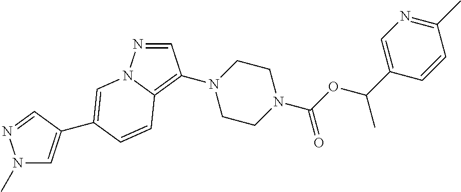 |
| 318 | 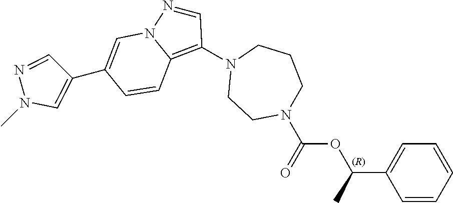 |
| 319 | 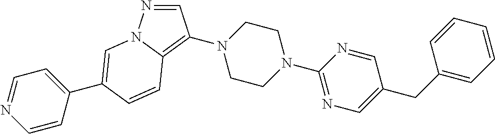 [SMILES: c1ccc(Cc2cnc(N3CCN(c4cnn5cc(-c6ccncc6)ccc45)CC3)nc2)cc1] |
| 320 | 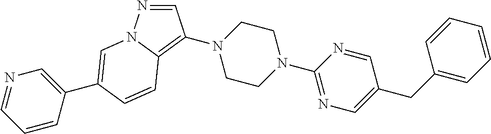 [SMILES: c1ccc(Cc2cnc(N3CCN(c4cnn5cc(-c6cccnc6)ccc45)CC3)nc2)cc1] |
| 321 | 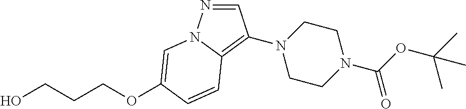 [SMILES: CC(C)(C)OC(=O)N1CCN(c2cnn3cc(OCCCO)ccc23)CC1] |
| 322 | 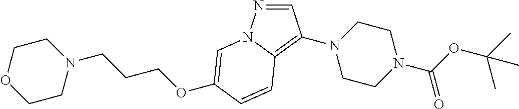 [SMILES: CC(C)(C)OC(=O)N1CCN(c2cnn3cc(OCCCN4CCOCC4)ccc23)CC1] |
| 323 | 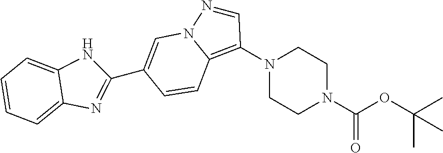 [SMILES: CC(C)(C)OC(=O)N1CCN(c2cnn3cc(-c4nc5ccccc5[nH]4)ccc23)CC1] |
| 324 | 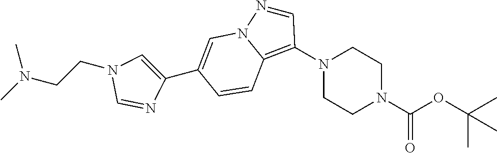 [SMILES: CN(C)CCn1cnc(-c2ccc3c(N4CCN(C(=O)OC(C)(C)C)CC4)cnn3c2)c1] |
| 325 | 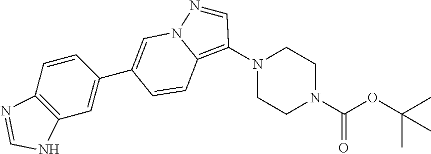 [SMILES: CC(C)(C)OC(=O)N1CCN(c2cnn3cc(-c4ccc5nc[nH]c5c4)ccc23)CC1] |

### II.B. Antibodies

In some aspects, a PDGFRα inhibitor comprises an antibody or fragment thereof. that is capable of inhibiting or reducing a PDGFRα activity or PDGFRα protein levels in a cell (e.g., OPC) (referred to herein as “antagonistic anti-PDGFRα antibody”).

Examples of suitable antagonistic antibodies that can target PDGFRα are known in the art. Non-limiting examples include: AF1062, (R&D Systems) WH005156M1 (Sigma-Aldrich), and 14-1401-82. (ThermoFisher Scientific) Additional examples of antagonistic anti-PDGFRα antibodies are provided in U.S. Pat. No. 8,574,578. Methods of humanizing non-human antibodies are well-known in the art.

### II.C. Gene Editing Tools

In some aspects, a PDGFRα inhibitor comprises a gene editing tool. As will be apparent to those skilled in the arts, when used as a gene editing tool, a PDGFRα inhibitor does not necessarily target and bind to a PDGFRα to inhibit its activity. Instead, in some aspects, a gene editing tool modifies a nervous system cell (e.g., OPC) such that the expression of PDGFRα on the nervous system cell (e.g., OPC) is inhibited (e.g., reduced, deleted or knocked-out), and thereby interfere with the PDGFRα activity of the cell. In some aspects, a PDGFRα inhibitor is capable of inhibiting the expression of PDGFRα on a nervous system cell (e.g., OPC) by at least 5%, at least 10%, at least 20%, at least 30%, at least 40%, at least 50%, at least 60%, at least 70%, at least 80%, at least 90%, or 100% compared to the expression of PDGFRα on a corresponding nervous system cell (e.g., OPC) that was not modified with the PDGFRα inhibitor. The protein expression of PDGFRα on the modified cells can be determined using any suitable methods known in the art. For example, in some aspects, whether the PDGFRα expression is inhibited can be determined using flow cytometry, e.g., by staining for expression using a fluorescently conjugated anti-PDGFRα antibody and comparing the fluorescent expression on the modified cells to that of control cells (e.g., corresponding cells that have not been modified). In some aspects, PDGFRα protein expression can be determined using a Western blot. In some aspects, the inhibition in PDGFRα expression can be determined at the gene level, e.g., with a PCR assay.

As used herein, the term “gene editing tool” refers to any system that can be used to alter the genome of a cell, tissue, or an organism and thereby, modify the expression of a gene of interest in the cell, tissue, or organism (e.g., to reduce or inhibit the expression of PDGFRα on nervous system cells). As used herein, the terms “gene of interest” and “target gene” can be used interchangeably and, in some aspects, refer to a gene (e.g., within a cell, tissue, or organism) which is modified by a gene editing tool disclosed herein (e.g., PDGFRα). In some aspects, the genetic alteration of the target genome can occur through the insertion, deletion, modification, or substitution of DNA in the target genome. Different tools for genome editing are known in the art. See, e.g., US Publ. No. 2017/0283830 A1.

As described herein, in some aspects, gene editing tools that are useful for the present disclosure include: a zinc-finger nuclease, meganuclease, transcription activator-like effector nuclease (TALEN), a CRISPR/Cas system, base editors, prime editors, AAV, miRNA, or any combination thereof.

### II.C.I. CRISPR/Cas System

In some aspects, the PDGFRα inhibitor comprises a CRISPR/Cas system that is capable of (or in some embodiments used for) inhibiting (e.g., reducing or knocking-out) the expression of PDGFRα on a nervous system cell (e.g., OPC).

In some aspects, a PDGFRα inhibitor comprises a Cas protein that is capable of (or in some embodiments used for) cleaving a region of the PDGFRα gene such that PDGFRα expression is inhibited in a nervous system cell (e.g., OPC). In some aspects, any known Cas protein can be used with the present disclosure. Non-limiting examples of Cas proteins that are useful for the present disclosure include: Cas1, Cas2, Cas3, Cas4, Cas5, Cas6, Cas7, Cas8, Cas9, Cas12, Cas13, CasX, CasY, and combinations thereof. In some aspects, a Cas protein useful for the present disclosure is from Streptococcus pyogenes. In some aspects, Cas protein from other species (e.g., Staphylococcus aureus) can be used with the present disclosure. In some aspects, the Cas protein is a Cas9 nuclease.

In some aspects, a PDGFRα inhibitor comprising a Cas protein (e.g., Cas9) can further comprise a guide RNA (gRNA). For instance, in some aspects, a Cas protein (e.g., Cas9) can be delivered to a cell as a complex protein, in which the Cas protein and the gRNA are associated with each other.

In some aspects, the PDGFRα inhibitor (e.g., Cas) can be introduced into the cell as a protein, which then passes through the nuclear membrane to enter the nucleus. In some aspects, the PDGFRα inhibitor can be introduced into the cell as mRNA, which is then translated prior to being delivered to the nucleus of the cell. In some aspects, the Cas protein (or any of the other gene editing tools described herein) can be formulated with a lipid to form lipid nanoparticles (LNPs).

### II.C.2. Meganuclease

In some aspects, the PDGFRα inhibitor that can be used with the present disclosure comprises a nuclease agent, such as a meganuclease system that is capable of (or in some embodiments used for) inhibiting (e.g., reducing, deleting or knocking-out) the expression of PDGFRα on a nervous system cell (e.g., OPC).

In constructing a meganuclease as a PDGFRα inhibitor, any meganuclease can be used herein, including, but not limited to, I-Scel, I-Scell, I-SceIII, I-SceIV, I-SceV, I-SecVI, I-SceVII, I-Ceul, I-CeuAIIP, I-Crel, I-CrepsbIP, I-CrepsbIIP, I-CrepsbIIIP, I-CrepsbIVP, I-TliI, I-PpoI, PI-PspI, F-Scel, F-SceII, F-SuvI, F-TevI, F-TevII, I-Amal, I-AniI, I-Chul, I-Cmoel, I-CpaI, I-CpaII, I-CsmI, I-Cvul, I-CvuAIP, I-DdiI, I-DdiII, I-DirI, I-Dmol, I-Hmul, I-HmuII, I-HsNIP, I-LlaI, I-MsoI, I-Naal, I-NanI, I-NcIIP, I-NgrIP, I-NitI, I-Njal, I-Nsp236IP, I-PakI, I-PboIP, I-PcuIP, I-PcuAI, I-PcuVI, I-PgrIP, I-PobIP, I-PorIIP, I-PbpIP, I-SpBetaIP, I-Scal, I-SexIP, I-SneIP, I-SpomI, I-SpomCP, I-SpomIP, I-SpomIIP, I-SquIP, I-Ssp6803I, I-SthPhiJP, I-SthPhiST3P, I-SthPhiSTe3bP, I-TdeIP, I-TevI, I-TevII, I-TevIII, I-UarAP, I-UarHGPAIP, I-UarHGPA13P, I-VinIP, I-ZbiIP, PI-Mtul, PI-MtuHIP, PI-MtuHIIP, PI-Pful, PI-PfuII, PI-PkoI, PI-PkoII, PI-Rma43812IP, PI-SpBetaIP, PI-Scel, PI-TfuI, PI-TfuII, PI-ThyI, PI-TliI, PI-TliII, or any active variants or fragments thereof.

### II.C.3. TALEN

In some aspects, a PDGFRα inhibitor comprises a nuclease agent, such as a Transcription Activator-Like Effector Nuclease (TALEN) that is capable of (or in some embodiments used for) inhibiting (e.g., reducing, deleting or knocking-out) the expression of PDGFRα on a nervous system cell (e.g., OPC).

Non-limiting examples of suitable TAL nucleases, and methods for preparing suitable TAL nucleases, are disclosed, e.g., in US Patent Application Nos. 2011/0239315 A1, 2011/0269234 A1, 2011/0145940 A1, 2003/0232410 A1, 2005/0208489 A1, 2005/0026157 A1, 2005/0064474 A1, 2006/0188987 A1, and 2006/0063231 A1.

In some aspects, TAL effector nucleases are engineered that cut in or near a target nucleic acid sequence in, e.g., a genomic locus of interest, wherein the target nucleic acid sequence is at or near a sequence to be modified by a targeting vector. The TAL nucleases suitable for use with the various methods and compositions provided herein include those that are specifically designed to bind at or near target nucleic acid sequences to be modified (e.g., PDGFRα) by targeting vectors as described herein.

### II.C.4. Zinc-Finger Nuclease (ZFN)

In some aspects, a PDGFRα inhibitor useful for the present disclosure comprises a nuclease agent, such as a zinc-finger nuclease (ZFN) system that is capable of (or in some embodiments used for) inhibiting (e.g., reducing, deleting or knocking-out) the expression of PDGFRα on a nervous system cell (e.g., OPC). Zinc finger-based systems comprise a fusion protein comprising two protein domains: a zinc finger DNA binding domain and an enzymatic domain.

In some aspects, a zinc finger binding domain comprises one or more zinc fingers. See, e.g., Miller et al., (1985) EMBO J. 4:1609-1614; Rhodes (1993) Scientific American February: 56-65; U.S. Pat. No. 6,453,242. In some aspects, the DNA-binding domains of individual ZFNs comprise between three and six individual zinc finger repeats and can each recognize between 9 and 18 basepairs.

Methods of engineering zinc finger binding domains to bind to a target sequence (e.g., encoding PDGFRα) are known in the art. See, for example, Beerli et al., (2002) Nature Biotechnol. 20:135-141; Pabo et al., (2001) Ann. Rev. Biochem. 70:313-340; Isalan et al., (2001) Nature Biotechnol. 19:656-660; Segal et al., (2001) Curr. Opin. Biotechnol. 12:632-637; Choo et al., (2000) Curr. Opin. Struct. Biol. 10:411-416; 2002-2003 Catalogue, New England Biolabs, Beverly, Mass.; and Belfort et al., (1997) Nucleic Acids Res. 25:3379-3388.

Exemplary restriction endonucleases (restriction enzymes) suitable for use as an enzymatic domain of the ZFPs described herein are present in many species and are capable of (and in some embodiments used for) sequence-specific binding to DNA (at a recognition site), and cleaving DNA at or near the site of binding. Certain restriction enzymes (e.g., Type IIS) cleave DNA at sites removed from the recognition site and have separable binding and cleavage domains. For example, the Type IIS enzyme FokI catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al., (1992) Proc. Natl. Acad. Sci. USA 89:4275-4279; Li et al., (1993) Proc. Natl. Acad. Sci. USA 90:2764-2768; Kim et al., (1994a) Proc. Natl. Acad. Sci. USA 91:883-887; Kim et al., (1994^{b}) J. Biol. Chem. 269:31,978-31,982.

### II.C.5. Interference RNA (RNAi)

In some aspects, a PDGFRα inhibitor that can be used with the present disclosure comprises an RNA interference molecule (RNAi) that is capable of (or in some embodiments used for) inhibiting (e.g., reducing or knocking-down) the expression of PDGFRα on a nervous system cell (e.g., OPC). As used herein, “RNAi” are RNA polynucleotide that mediates the decreased expression of an endogenous target gene product by degradation of a target mRNA through endogenous gene silencing pathways (e.g., Dicer and RNA-induced silencing complex (RISC)). Non-limiting examples of RNAi agents include micro RNAs (miRNAs), short hair-pin RNAs (shRNAs), small interfering RNAs (siRNAs), RNA aptamers, or combinations thereof.

In some aspects, a PDGFRα inhibitor comprises one or more miRNAs. “miRNAs” refer to naturally occurring, small non-coding RNA molecules of about 21-25 nucleotides in length. miRNAs can downregulate (e.g., decrease) expression of an endogenous target gene product (e.g., PDGFRα) through translational repression, cleavage of the mRNA, and/or deadenylation.

In some aspects, a PDGFRα inhibitor comprises one or more shRNAs. “shRNAs” (or “short hairpin RNA” molecules) refer to an RNA sequence comprising a double-stranded region and a loop region at one end forming a hairpin loop, which can be used to reduce and/or silence a gene expression

In some aspects, a PDGFRα inhibitor comprises one or more siRNAs that is capable of (or in some embodiments used for) inhibiting (e.g., reducing or knocking-down) the expression of PDGFRα on a nervous system cell (e.g., OPC). “siRNAs” refer to double stranded RNA molecules typically about 21-23 nucleotides in length.

### III. Pharmaceutical Compositions

Provided herein are compositions comprising a PDGFRα inhibitor of the present disclosure (e.g., small molecules or antibodies) having the desired degree of purity, and a pharmaceutically acceptable carrier or excipient, in a form suitable for administration to a subject. Pharmaceutically acceptable excipients or carriers can be determined in part by the particular composition being administered, as well as by the particular method used to administer the composition. Accordingly, there is a wide variety of suitable formulations of pharmaceutical compositions (See, e.g., Remington, 23^{rd} Edition, The Science and Practice of Pharmacy, editor: A. Adejare, 2020, Adademic Press.). The pharmaceutical compositions are generally formulated sterile and in full compliance with all Good Manufacturing Practice (GMP) regulations of the U.S. Food and Drug Administration.

In some aspects, a pharmaceutical composition comprises a PDGFRα inhibitor described herein, and a pharmaceutically acceptable carrier. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN®, PLURONICS® or polyethylene glycol (PEG).

In some aspects, a pharmaceutical composition disclosed herein comprises one or more additional components selected from: a bulking agent, stabilizing agent, surfactant, buffering agent, or combinations thereof.

Buffering agents useful for the current disclosure can be a weak acid or base used to maintain the acidity (pH) of a solution near a chosen value after the addition of another acid or base. Suitable buffering agents can maximize the stability of the pharmaceutical compositions by maintaining pH control of the composition. Suitable buffering agents can also ensure physiological compatibility or optimize solubility. Rheology, viscosity and other properties can also be dependent on the pH of the composition. Common buffering agents include, but are not limited to, a Tris buffer, a Tris-Cl buffer, a histidine buffer, a TAE buffer, a HEPES buffer, a TBE buffer, a sodium phosphate buffer, a MES buffer, an ammonium sulfate buffer, a potassium phosphate buffer, a potassium thiocyanate buffer, a succinate buffer, a tartrate buffer, a DIPSO buffer, a HEPPSO buffer, a POPSO buffer, a PIPES buffer, a PBS buffer, a MOPS buffer, an acetate buffer, a phosphate buffer, a cacodylate buffer, a glycine buffer, a sulfate buffer, an imidazole buffer, a guanidine hydrochloride buffer, a phosphate-citrate buffer, a borate buffer, a malonate buffer, a 3-picoline buffer, a 2-picoline buffer, a 4-picoline buffer, a 3,5-lutidine buffer, a 3,4-lutidine buffer, a 2,4-lutidine buffer, a Aces, a diethylmalonate buffer, a N-methylimidazole buffer, a 1,2-dimethylimidazole buffer, a TAPS buffer, a bis-Tris buffer, a L-arginine buffer, a lactate buffer, a glycolate buffer, or combinations thereof.

In some aspects, a pharmaceutical composition disclosed herein further comprises a bulking agent. Bulking agents can be added to a pharmaceutical product in order to add volume and mass to the product, thereby facilitating precise metering and handling thereof. Bulking agents that can be used with the present disclosure include, but are not limited to, sodium chloride (NaCl), mannitol, glycine, alanine, or combinations thereof.

In some aspects, a pharmaceutical composition disclosed herein can also comprise a stabilizing agent. Non-limiting examples of stabilizing agents that can be used with the present disclosure include: sucrose, trehalose, raffinose, arginine, or combinations thereof.

In some aspects, a pharmaceutical composition disclosed herein comprises a surfactant. In some aspects, the surfactant can be selected from the following: alkyl ethoxylate, nonylphenol ethoxylate, amine ethoxylate, polyethylene oxide, polypropylene oxide, fatty alcohols such as cetyl alcohol or oleyl alcohol, cocamide MEA, cocamide DEA, polysorbates, dodecyl dimethylamine oxide, or combinations thereof. In some aspects, the surfactant is polysorbate 20 or polysorbate 80.

In some aspects, a pharmaceutical composition disclosed herein (e.g., comprising a PDGFRα inhibitor) further comprises an amino acid. In some aspects, the amino acid is selected from arginine, glutamate, glycine, histidine, or combinations thereof. In some aspects, the composition further comprises a sugar alcohol. Non-limiting examples of sugar alcohol includes: sorbitol, xylitol, maltitol, mannitol, or combinations thereof.

A pharmaceutical composition disclosed herein (e.g., comprising a PDGFRα inhibitor) can be formulated for any route of administration to a subject. Specific examples of routes of administration include intramuscularly, cutaneously, subcutaneously, ophthalmic, intravenously, intraperitoneally, intradermally, intraorbitally, intracerebrally, intracranially, intraspinally, intraventricularly, intrathecally, intracapsularly, orally, pulmonarily, intranasally, intra-arterially, intralymphatically, periocularly, topically, rectally, vaginally, or intratumorally or via intratympanic injection. Parenteral administration, characterized by, e.g., cutaneous, subcutaneous, intramuscular, or intravenous injection, is also contemplated herein.

Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. In addition, if desired, the pharmaceutical compositions to be administered can also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances. Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations can be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions includes EDTA. Pharmaceutical carriers also include ethyl alcohol, polyethylene glycol and propylene glycol for water miscible vehicles; and sodium hydroxide, hydrochloric acid, citric acid or lactic acid for pH adjustment.

Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions can be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Topical mixtures comprising an antibody are prepared as described for the local and systemic administration. The resulting mixture can be a solution, suspension, emulsions or the like and can be formulated as creams, gels, ointments, emulsions, solutions, elixirs, lotions, suspensions, tinctures, pastes, foams, aerosols, irrigations, sprays, suppositories, bandages, dermal patches, or any other formulations suitable for topical administration.

A therapeutic agent described herein (e.g., PDGFRα inhibitor) can be formulated as an aerosol for topical application, such as by inhalation (see, e.g., U.S. Pat. Nos. 4,044,126; 4,414,209; and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflations, alone or in combination with an inert carrier such as lactose. In such a case, the particles of the formulation can have diameters of less than about 50 microns, e.g., less than about 10 microns.

A therapeutic agent disclosed herein (e.g., PDGFRα inhibitor) can be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies.

Transdermal patches, including iontophoretic and electrophoretic devices, are well known to those of skill in the art, and can be used to administer a therapeutic agent (e.g., those disclosed herein). For example, such patches are disclosed in U.S. Pat. Nos. 6,267,983; 6,261,595; 6,256,533; 6,167,301; 6,024,975; 6,010715; 5,985,317; 5,983,134; 5,948,433; and 5,860,957.

In some aspects, a pharmaceutical composition comprising a therapeutic agent described herein (e.g., PDGFRα inhibitor) is a lyophilized powder, which can be reconstituted for administration as solutions, emulsions and other mixtures. It can also be reconstituted and formulated as solids or gels. The lyophilized powder is prepared by dissolving an antibody or antigen-binding portion thereof described herein, or a pharmaceutically acceptable derivative thereof, in a suitable solvent. In some aspects, the lyophilized powder is sterile. The solvent can contain an excipient which improves the stability or other pharmacological component of the powder or reconstituted solution, prepared from the powder. Excipients that can be used include, but are not limited to, dextrose, sorbitol, fructose, corn syrup, xylitol, glycerin, glucose, sucrose or other suitable agent. The solvent can also contain a buffer, such as citrate, sodium or potassium phosphate or other such buffer known to those of skill in the art at, in some aspects, about neutral pH. Subsequent sterile filtration of the solution followed by lyophilization under standard conditions known to those of skill in the art provides the desired formulation. In some aspects, the resulting solution can be apportioned into vials for lyophilization. Each vial can contain a single dosage or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable carrier. The precise amount depends upon the selected compound. Such amount can be empirically determined.

Pharmaceutical compositions provided herein (e.g., comprising a PDGFRα inhibitor) can also be formulated to be targeted to a particular tissue, receptor, or other area of the body of the subject to be treated. Many such targeting methods are known to those of skill in the art. All such targeting methods are contemplated herein for use in the instant compositions. For non-limiting examples of targeting methods, see, e.g., U.S. Pat. Nos. 6,316,652; 6,274,552; 6,271,359; 6,253,872; 6,139,865; 6,131,570; 6,120,751; 6,071,495; 6,060,082; 6,048,736; 6,039,975; 6,004,534; 5,985,307; 5,972,366; 5,900,252; 5,840,674; 5,759,542; and 5,709,874.

The pharmaceutical compositions to be used for in vivo administration can be sterile. This can be accomplished, for example, by filtration through, e.g., sterile filtration membranes.

### IV. Kits

Also provided herein are kits comprising one or more PDGFRα inhibitors described herein. In some aspects, provided herein is a pharmaceutical pack or kit comprising one or more containers filled with one or more of the ingredients of the pharmaceutical compositions described herein, such as one or more PDGFRα inhibitors provided herein, and optionally, an instruction for use. In some aspects, the kits contain a pharmaceutical composition described herein (e.g., a PDGFRα inhibitor) and any prophylactic or therapeutic agent, such as those described herein.

### V. Nucleic Acids, Vectors, Host Cells

Further aspects described herein pertains to one or more nucleic acid molecules that encode a therapeutic agent described herein (e.g., a PDGFRα inhibitor, e.g., a gene editing tool described herein). The nucleic acids can be present in whole cells, in a cell lysate, or in a partially purified or substantially pure form. A nucleic acid is “isolated” or “rendered substantially pure” when purified away from other cellular components or other contaminants, e.g., other cellular nucleic acids (e.g., other chromosomal DNA, e.g., the chromosomal DNA that is linked to the isolated DNA in nature) or proteins, by standard techniques, including alkaline/SDS treatment, CsCl banding, column chromatography, restriction enzymes, agarose gel electrophoresis and others well known in the art. A nucleic acid described herein can be, for example, DNA or RNA and can or cannot contain intronic sequences. In some aspects, the nucleic acid is a cDNA molecule. Nucleic acids described herein can be obtained using standard molecular biology techniques known in the art.

In some aspects, the present disclosure provides a vector comprising an isolated nucleic acid molecule encoding a therapeutic agent disclosed herein (e.g., PDGFRα inhibitor, e.g., a gene editing tool described herein).

Suitable vectors for the disclosure include expression vectors, viral vectors, and plasmid vectors. In some aspects, the vector is a viral vector.

As used herein, viral vectors include, but are not limited to, nucleic acid sequences from the following viruses: retrovirus, such as Moloney murine leukemia virus, Harvey murine sarcoma virus, murine mammary tumor virus, and Rous sarcoma virus; lentivirus; adenovirus; adeno-associated virus; SV40-type viruses; polyomaviruses; Epstein-Barr viruses; papilloma viruses; herpes virus; vaccinia virus; polio virus; and RNA virus such as a retrovirus. One can readily employ other vectors well-known in the art. Certain viral vectors are based on non-cytopathic eukaryotic viruses in which non-essential genes have been replaced with the gene of interest. Non-cytopathic viruses include retroviruses, the life cycle of which involves reverse transcription of genomic viral RNA into DNA with subsequent proviral integration into host cellular DNA.

In some aspects, a vector is derived from an adeno-associated virus. In some aspects, a vector is derived from a lentivirus. Examples of the lentiviral vectors are disclosed in WO9931251, WO9712622, WO9817815, WO9817816, and WO9818934.

Other vectors include plasmid vectors. Plasmid vectors have been extensively described in the art and are well-known to those of skill in the art. See, e.g., Sambrook et al., Molecular Cloning: A Laboratory Manual, Second Edition, Cold Spring Harbor Laboratory Press, 1989. In the last few years, plasmid vectors have been found to be particularly advantageous for delivering genes to cells in vivo because of their inability to replicate within and integrate into a host genome. These plasmids, however, having a promoter compatible with the host cell, can express a peptide from a gene operably encoded within the plasmid. Some commonly used plasmids available from commercial suppliers include pBR322, pUC18, pUC19, various pcDNA plasmids, pRC/CMV, various pCMV plasmids, pSV40, and pBlueScript. Additional examples of specific plasmids include pcDNA3.1, catalog number V79020; pcDNA3.1/hygro, catalog number V87020; pcDNA4/myc-His, catalog number V86320; and pBudCE4.1, catalog number V53220, all from Invitrogen (Carlsbad, CA.). Other plasmids are well-known to those of ordinary skill in the art. Additionally, plasmids can be custom designed using standard molecular biology techniques to remove and/or add specific fragments of DNA.

Also encompassed by the present disclosure is a method for making a molecule disclosed herein (e.g., a PDGFRα inhibitor). In some aspects, such a method can comprise expressing the molecule (e.g., PDGFRα inhibitor) in a cell comprising a nucleic acid molecule encoding the molecule. Host cells comprising these nucleotide sequences are encompassed herein. Non-limiting examples of host cell that can be used include immortal hybridoma cell, NS/0 myeloma cell, 293 cell, Chinese hamster ovary (CHO) cell, HeLa cell, human amniotic fluid-derived cell (CapT cell), COS cell, or combinations thereof.

### VI. Assays

Disclosed herein is an assay (see Examples 57 and 58) that can be used to assess the differentiation status of a nervous system cell (e.g., OPC). Compared to in vitro differentiation assays available in the art (see, e.g., Schott et al., J Vis Exp 108:53764 (February 2016); and Deshmukh et al., Nature 502 (7471): 327-332 (October 2013)), the present assay provides certain distinct advantages. For example, the assays in the art generally involve an initial step in which nervous system cells (e.g., OPCs) are cultured in a proliferation medium comprising PDGF. See, e.g., Deshmukh et al., page 10, 1^{st} paragraph. Once optimal confluency is reached, the nervous system cells (e.g., OPCs) are then contacted with a test compound in a differentiation medium that comprises reduced or no PDGF, and the effect of the test compound on the differentiation of the nervous system cell is assessed. Id. While reduced or lack of PDGF can help initiate the differentiation process, the present disclosure identifies that such a condition can also narrow the timeframe during which the effect of the test compound can be accurately assessed. For example, if the nervous system cells (e.g., OPCs) have undergone substantial spontaneous differentiation due to the reduced or lack of PDGF, it would be difficult to assess whether the test compound had any effect on the differentiation process. Additionally, there is PDGF signalling in the natural physiological situation occurring in vivo and PDGF signalling is unlikely to drop immediately before treatment. Accordingly, contrary to the assays in the art, the in vitro differentiation assay described herein contains the same amount of PDGF throughout the entire assay (i.e., both during the initial proliferation of the nervous system cells (e.g., OPCs) and during the differentiation when the effect of the test compounds is assessed). In some aspects, the concentration of PDGF present throughout the entire assay is at least about 10 ng/mL. In some aspects, the amount of PDGF present in the assay during the differentiation of the nervous system cells (e.g., OPCs) is greater than the amount present during the initial proliferation. By providing the same or higher amount of PDGF during the differentiation process (as compared to the initial proliferation), the present disclosure identifies that the effect of a test compound on nervous system cell (e.g., OPC) differentiation can be accurately assessed. For example, because the amount of PDGF present is the same throughout the assay, there is minimal concern for any effects due to spontaneous differentiation. Any differentiation observed is most likely due to the test compound itself (e.g., PDGFRα inhibitors described herein).

Additionally, the in vitro differentiation assays available in the art generally use proteins, such as myelin basic protein (MBP), as a readout for nervous system cell differentiation. See, e.g., Deshmukh et al., page 11, 1^{st} full paragraph. However, as explained elsewhere in the present disclosure, such protein are generally only expressed on fully differentiated/matured nervous system cells (e.g., oligodendrocytes and neurons). In contrast, as described in Example 240, the in vitro differentiation assay of the present disclosure uses GPR17 as a marker for differentiation. GPR17 is preferentially expressed in actively differentiating nervous system cells (e.g., oligodendrocytes), allowing them to be distinguished from precursors and completely mature cells. Therefore, with the in vitro differentiation assay described herein, in some aspects, it is possible to determine whether a test compound (e.g., PDGFRα inhibitors described herein) has an effect on nervous system cell differentiation, not only more accurately, but also much more quickly compared to other assays available in the art both in vitro and in vivo.

### VII. Methods of the Disclosure

As demonstrated herein, PDGFRα inhibitors of the present disclosure (e.g., small molecules and antibodies) are useful in reducing or inhibiting PDGFRα activity, which, in some aspects, can promote the myelination of an axon and/or remyelination of a demyelinated neuronal axon.

In some aspects, the present disclosure relates to methods of inhibiting or reducing PDGFRα activity in a cell or tissue, e.g., of a subject in need thereof, comprising contacting the cell or tissue with a PDGFRα inhibitor described herein. In some aspects, after the contacting, PDGFRα kinase activity in the cell or tissue is inhibited by at least 5%, at least 10%, at least 20%, at least 30%, at least 40%, at least 50%, at least 60%, at least 70%, at least 80%, at least 90%, or 100% compared to a reference (e.g., PDGFRα activity in the cell prior to the contacting and/or PDGFRα activity in a corresponding cell that was not contacted with the PDGFRα inhibitor). The PDGFRα activity status of a cell can be determined using any suitable method known in the art. In some aspects, PDGFRα activity can be determined by measuring the corresponding gene expression level, e.g., by qRT-PCR. In some aspects, PDGFRα activity can be determined by measuring the PDGFRα protein level, e.g., by immunoblotting. In some aspects, the PDGFRα activity can be determined by measuring receptor phosphorylation, e.g., by western blotting. An exemplary method of assaying such an activity, a purified protein enzyme assay, is provided in Example 239 (also referred to herein as the “Promega Assay”). In some aspects, the enzymatic PDGFRα kinase assay comprises 20 ng of purified PDGFRα protein, 150 uM of ATP and 1 ug of substrate, Poly (Glu4Tyr1) in a volume of 15 ul. The potency of a compound can be determined using such assays. In general, compounds most useful in the methods of the present disclosure have an IC₅₀ below 10,000 nM (e.g., less than 9,000 nM, less than 8,000 nM, less than 7,000 nM, less than 6,000 nM, less than 5,000 nM, less than 4,000 nM, less than 3,000 nM, less than 2,000 nM, less than 1,000 nM, less than 900 nM, less than 800 nM, less than 700 nM, less than 600 nM, less than 500 nM, less than 400 nM, less than 300 nM, less than 200 nM, less than 100 nM, less than 50 nM, less than 25 nM, less than 10 nM, or less than 1 nM, for instance, less than 500 nM) in such an assay. As described herein, in some aspects, the cell is an oligodendrocyte progenitor cell (OPC).

As both described and demonstrated herein, inhibiting or reducing PDGFRα activity can promote the myelination of a neuron (e.g., an axon of a neuron,), e.g., by promoting the differentiation of an OPC into an oligodendrocyte that, in turn, myelinates the axon. Similarly, inhibiting or reducing PDGFRα activity can also promote the remyelination of a demyelinated neuronal axon. And, as will be apparent to those skilled in the art, in some aspects, remyelination can repair damage to demyelinated (e.g., hypomyelinated) axons, and thereby, aid in reducing axonal loss.

Accordingly, in some aspects, provided herein is a method of promoting the myelination of an axon, e.g., in a subject in need thereof (e.g., subject suffering from or at risk of developing a demyelinating disease), the method comprising contacting an OPC with an effective amount of a PDGFRα inhibitor described herein, wherein the contacting results in the OPC to differentiate into an oligodendrocyte, and wherein the oligodendrocyte is capable of (and in some embodiments used for) promoting the myelination of the axon. In some aspects, after the contacting, the number of neurons with axons that are myelinated is increased by at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 25-fold, at least 30-fold, at least 35-fold, at least 40-fold, at least 45-fold, or at least 50-fold compared to a reference (e.g., the number of neurons with myelinated axons in the subject prior to the administration and/or the number of neurons with myelinated axons in a corresponding subject that did not receive the administration of the PDGFRα inhibitor). The increase in the number of neurons with axons that are myelinated can be determined using any suitable methods known in the art or described herein. In some aspects, the increase in the number of neurons with axons that are myelinated can be determined by visualizing and/or quantifying the expression of a marker associated with myelinated neurons. For example, in some aspects, the marker associated with myelinated neurons comprises myelin basic protein (MBP), myelin oligodendrocyte glycoprotein (MOG), oligodendrocyte-specific protein/claudin-11, CNPase, or a combination thereof.

In some aspects, also provided herein is a method of promoting remyelination of an axon of a neuron (e.g., demyelinated neuronal axon in a subject in need thereof), comprising contacting an OPC with an effective amount of any of the PDGFRα inhibitors described herein, wherein the contacting results in the OPC to differentiate into an oligodendrocyte, and wherein the oligodendrocyte is capable of (and in some embodiments used for) promoting the remyelination of the demyelinated neuronal axon. In some aspects, after the contacting, the number of demyelinated axons that are myelinated is increased by at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 25-fold, at least 30-fold, at least 35-fold, at least 40-fold, at least 45-fold, or at least 50-fold compared to a reference (e.g., the number of demyelinated neuronal axons in the subject prior to the administration and/or the amount of demyelinated neuronal axons in a corresponding subject that did not receive the administration of the PDGFRα inhibitor). The increase in the number of demyelinated axons that are myelinated can be determined using any suitable methods known in the art or described herein. In some aspects, the increase in the number of demyelinated axons that are myelinated can be determined by visualizing and/or quantifying the expression of a marker associated with myelinated neurons. For example, in some aspects, the marker associated with myelinated neurons comprises myelin basic protein (MBP), myelin oligodendrocyte glycoprotein (MOG), oligodendrocyte-specific protein/claudin-11, CNPase, or a combination thereof.

In some aspects, provided herein is a method of reducing the demyelination of a myelinated axon (e.g., reducing the rate at which the myelinated axons become demyelinated) in a subject in need thereof, comprising contacting an OPC with an effective amount of any of the PDGFRα inhibitors described herein, wherein the contacting results in the OPC to differentiate into an oligodendrocyte, and wherein the oligodendrocyte is capable of (and in some embodiments used for) reducing the demyelination of the myelinated axon. In some aspects, after the contacting, demyelination of a myelinated axon in the subject is reduced by at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 25-fold, at least 30-fold, at least 35-fold, at least 40-fold, at least 45-fold, or at least 50-fold compared to a reference (e.g., demyelination of a myelinated axon in a corresponding subject that did not receive an administration of the PDGFRα inhibitor). The decrease in the number of neurons with axons that are demyelinated can be determined using any suitable methods known in the art or described herein. In some aspects, the decrease in the number of neurons with axons that are demyelinated can be determined by visualizing and/or quantifying the expression of a marker associated with myelinated neurons. For example, in some aspects, the marker associated with myelinated neurons comprises myelin basic protein (MBP), myelin oligodendrocyte glycoprotein (MOG), oligodendrocyte-specific protein/claudin-11, CNPase, or a combination thereof.

In any of the methods provided herein, in some aspects, the contacting can occur ex vivo (e.g., OPCs of a subject can be isolated and contacted with a PDGFRα inhibitor in vitro to assess the therapeutic efficacy of the inhibitor). In some aspects, the contacting occurs in vivo (e.g., in a subject in need thereof). Where the contacting occurs in vivo, the methods can further comprise administering to the subject an effective amount of any of the PDGFRα inhibitors described herein.

Not to be bound by any one theory, in some aspects, after administration to a subject, a PDGFRα inhibitor described herein is capable of (and in some embodiments used for) migrating into the CNS of the subject (e.g., by crossing the blood-brain barrier) and interacting with the nervous system cells present within the CNS. For instance, as described and demonstrated herein, PDGFRα inhibitors of the present disclosure are capable of (and in some embodiments used for) targeting PDGFRα expressed on an OPC, and thereby inhibit or reduce the PDGFRα activity of the OPC. In some aspects, this results in the activation of the OPC and the subsequent differentiation of the OPC into an oligodendrocyte. Accordingly, in some aspects, provided herein is a method of activating an OPC within the CNS of a subject in need thereof, the method comprising administering to the subject any of the PDGFRα inhibitors described herein.

As described herein, the myelin sheath which surrounds the neuronal axons can affect various functions of neurons. For example, in some aspects, by increasing the myelination of neuronal axons, PDGFRα inhibitors described herein can increase the rate of transmission of electrical impulses (i.e., nerve signals or action potentials) along the axon of a neuron. Accordingly, in some aspects, the present disclosure provides a method of increasing electrical impulse transmission along the axon of a neuron, comprising contacting an OPC with an effective amount of any of the PDGFRα inhibitors described herein, wherein the contacting results in the OPC to differentiate into an oligodendrocyte, and wherein the oligodendrocyte is capable of (and in some embodiments used for) myelinating the axon of the neuron, and thereby increase the electrical impulse transmission. In some aspects, after contacting the OPC, the electrical impulse transmission along the axon of the neuron is increased by at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 25-fold, at least 30-fold, at least 35-fold, at least 40-fold, at least 45-fold, or at least 50-fold compared to a reference (e.g., electrical impulse transmission across the axon prior to or in the absence of contact with the PDGFRα inhibitors). Action potential conduction velocities can be measured as compound action potentials across the corpus callosum in vitro or by auditory or visual evoked potential recording in vivo. Remyelination can enhance axonal health and prevent axonal degradation in a diseased state. Non-limiting examples of methods useful for measuring such properties of a neuronal axon are described in, e.g., Li et al., PLOS One 11 (11): e0165637 (November 2016); Maheras et al., Sci Rep 8 (1): 3798 (February 2018); and Alqudah et al., Audiol Neurootol 23 (1): 20-31 (2018).

As is apparent from the present disclosure, the PDGFRα inhibitors described herein can be useful in the treatment of a demyelinating disease, e.g., by inducing the differentiation of an OPC into an oligodendrocyte, thereby promoting the remyelination of neuronal axons. Accordingly, in some aspects, the present disclosure is directed to a method of treating a demyelinating disease in a subject in need thereof, comprising administering to the subject a therapeutically effective amount of a PDGFRα inhibitor described herein. References to a method of treating a demyelinating disease as described herein also refer to the agents and compositions as described herein for use in treating a demyelinating disease.

The PDGFRα inhibitors provided herein can be used to treat a wide range of demyelinating diseases (including those that are associated with demyelination and/or hypomyelination). In some aspects, the demyelinating disease that can be treated with the present disclosure comprise those that are characterized by demyelination of one or more cells within the CNS. Non-limiting examples of demyelinating disease that can be treated with the present disclosure include: acute disseminated encephalomyelitis (ADEM), acute hemorrhagic leukoencephalitis, acute optic neuritis, acute transverse myelitis, adrenoleukodystrophy, adrenomyeloneuropathy, Alexander Alzheimer's disease, aminoacidurias, amyotrophic lateral sclerosis, anti-MAG peripheral neuropathy, anti-MOG associated spectrum, Balo concentric sclerosis, brain injury, CAMFAK Syndrome, Canavan disease, carbon monoxide toxicity, central pontine myelinolysis, cerebral hypoxia, cerebral ischemia, Charcot-Marie-Tooth disease, chronic inflammatory demyelinating polyneuropathy, chronic relapsing inflammatory optic neuritis (CRION), chronic traumatic encephalopathy, clinically isolated syndrome (CIS), congenital cataract, copper deficiency associated condition, delayed post-hypoxic leukoencephalopathy, diffuse cerebral sclerosis of Schilder, diffuse myelinoclastic sclerosis, extrapontine myelinolysis Gaucher disease, Guillain-Barré syndrome, hereditary neuropathy, hereditary neuropathy with liability to pressure palsy, HTLV-1-associated myelopathy, Hurler syndrome, hypomyelination, hypoxic brain injury, Krabbe disease, Leber hereditary optic atrophy and related mitochondrial disorders, leukodystrophic disorders, Marburg multiple sclerosis, Marchiafava-Bignami disease, metachromatic leukodystrophy, multiple sclerosis, multiple system atrophy, myelinoclastic disorders, myelopathy, nerve injury, neuromyelitis optica (NMO), Niemann-Pick disease, optic neuropathy, optic-spinal multiple sclerosis, osmotic demyelination syndrome, Parkinson's disease, Pelizaeus-Merzbacher disease, periventricular leukomalacia, peripheral neuropathy, phenylketonuria, primary progressive multiple sclerosis (PPMS), progressive inflammatory neuropathy, progressive multifocal leukoencephalopathy, progressive subcortical ischemic demyelination, progressive-onset multiple sclerosis, relapsing-onset multiple sclerosis, relapsing-remitting multiple sclerosis (RRMS), reperfusion injury, Schilder disease, secondary progressive multiple sclerosis (SPMS), solitary sclerosis, spinal cord injury, subacute sclerosing panencephalitis, Tabes dorsalis, Tay-Sachs disease, transverse myelitis, traumatic brain injury, tropical spastic paraparesis, tumefactive multiple sclerosis, vitamin B¹² deficiency, cerebral palsy, and combinations thereof. In some aspects, a disease that can be treated with the present disclosure comprises a tumor associated with abnormal (e.g., increased) PDGF activity. Such a tumor is referred to herein as “PDGF-associated tumor.” Accordingly, in some aspects, provided herein is a method of treating a PDGF-associated tumor in a subject in need thereof, comprising administering to the subject any of the PDGFRα inhibitor described herein. Non-limiting example of a PDGF-associated tumor comprises oligodendroglioma.

In some aspects, the demyelinating disease that can be treated with a PDGFRα inhibitor described herein comprises multiple sclerosis. As used herein, the term “multiple sclerosis” (MS) refers to a chronic and often disabling disease of the central nervous system characterized by the progressive destruction of the myelin sheath. Multiple sclerosis is generally diagnosed as one of four internationally recognized categories or stages of MS: (1) primary progressive multiple sclerosis (PPMS), (2) relapsing-remitting multiple sclerosis (RRMS), (3) secondary progressive multiple sclerosis (SPMS), and (4) progressive relapsing multiple sclerosis. Standards for diagnosis are known to those of skill in the art and exemplary diagnostic criteria are described in, e.g., “Merck Manual, Professional Version” (www.merckmanuals.com/professional/neurologic-disorders/demyelinating-disorders/multiple-sclerosis-ms). Unless indicated otherwise, the term “multiple sclerosis” encompasses all of the different categories of MS. Accordingly, in some aspects, the PDGFRα inhibitors of the present disclosure can be used to treat all types of MS.

In some aspects, a PDGFRα inhibitor of the present disclosure can be used to treat clinically isolated syndrome (CIS). In some aspects, a PDGFRα inhibitor of the present disclosure can be used to treat radiologically isolated syndrome (RIS).

In some aspects, provided herein is a method of treating a relapsing form of multiple sclerosis in a subject in need thereof, the method comprising administering to the subject a therapeutically effective amount of any of the PDGFRα inhibitors provided herein.

In some aspects, the demyelinating disease that can be treated with the present disclosure is an optic neuritis. Accordingly, in some aspects, a PDGFRα inhibitor is used to ameliorate optic neuritis, e.g., optic neuritis that is due to multiple sclerosis.

As is apparent from the present disclosure, by promoting the myelination of an axon and/or the remyelination of a demyelinated axon, PDGFRα inhibitors of the present disclosure can help restore nervous system cell function and thereby, reduce and/or alleviate one or more symptoms associated with a demyelinating disease. Accordingly, in some aspects, treating a demyelinating disease comprises reducing one or more symptoms associated with the demyelinating disease. Non-limiting examples of such symptoms include: fatigue, dizziness, malaise, elevated fever and high body temperature, extreme sensitivity to cold in the hands and feet, weakness and stiffness in muscles and joints, weight changes, digestive or gastrointestinal problems, low blood pressure, high blood pressure, irritability, anxiety, depression, blurred vision, double vision, ataxia, clonus, spasms, dysarthria, weakness, clumsiness, hand paralysis, hemiparesis, genital anesthesia, sexual dysfunction, incoordination, paresthesias, ocular paralysis, impaired muscle coordination, loss of sensation, tingling, numbness, pain, impaired vision, neurological symptoms, unsteady gait, balance problems, dizziness, spastic paraparesis, incontinence, hearing problems, speech problems, loss of olfaction, agusia, or combinations thereof.

Moreover, PDGFRα inhibitors described herein can also be used to prevent or delay the onset of one or more symptoms associated with a demyelinating disease. With many demyelinating diseases, particularly during the early stages, a subject may not exhibit any apparent symptoms of the disease. For instance, in some aspects, the damage to the myelin sheaths may not be as severe, such that the oligodendrocytes present within the CNS of the subject can adequately repair any damages. However, as is the case with most demyelinating diseases (e.g., multiple sclerosis), the damage to the myelin sheaths can become more severe, resulting in the manifestation of one or more symptoms of the disease. In some aspects, by administering a PDGFRα inhibitor described herein to the subject when the damage to the myelin sheath is still minor, the manifestation of the one or more symptoms of the disease can be prevented or delayed. In some aspects, compared to a reference subject (e.g., corresponding subject that did not receive an administration of the PDGFRα inhibitor), the manifestation of the one or more symptoms of the disease is delayed by at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 25-fold, at least 30-fold, at least 35-fold, at least 40-fold, at least 45-fold, or at least 50-fold. In some aspects, compared to the reference subject, the subject treated with a PDGFRα inhibitor described herein exhibits less number of symptoms and/or the symptoms are reduced in severity.

Whether a subject exhibits one or more symptoms associated with a demyelinating disease can be assessed using any suitable approaches known in the art. In some aspects, whether a subject exhibits the one or more symptoms associated with a demyelinating disease can be determined by assessing the subject's performance in one or more of the following tests: a visual evoked potential (VEP) test, a multifocal visual evoked potential (mfVEP) test, a low contrast visual acuity (LC-VA) test, a magnetic resonance imaging (MRI) (e.g., magnetic transfer resonance, myelin water fraction (MWF), and quantitative susceptibility mapping (QSM)), an electromyography (EMG), a nerve conduction velocity (NCV) test, an Extended Disability Status Scale (EDSS), a timed walk test (e.g., timed 25-foot walk), a Nine-Hole Peg Test (9HPT), an ocular coherence tomograph (OCT), a quality of life measure test (e.g., Multiple Sclerosis Quality of Life-54 and Vision-Related Quality of Life), cognitive assessment (e.g., Montreal Cognitive Assessment), or combinations thereof. In some aspects, whether a subject exhibits one or more symptoms associated with a demyelinating disease is determined by assessing the subject's performance in a LC-VA test. In some aspects, whether a subject exhibits one or more symptoms associated with a demyelinating disease is determined by assessing the subject's performance in a cognitive assessment.

In some aspects, an improved performance as compared to a reference performance (e.g., a corresponding subject's performance in the test where the subject was not treated with a PDGFRα inhibitor described herein and/or the subject's performance prior to the treatment with a PDGFRα inhibitor) indicates that the one or more symptoms associated with a demyelinating disease is reduced or alleviated in the subject. In some aspects, a comparable or reduced performance as compared to a reference performance (e.g., a corresponding subject's performance in the test where the subject was not treated with a PDGFRα inhibitor described herein and/or the subject's performance prior to the treatment with a PDGFRα inhibitor) indicates that the one or more symptoms associated with a demyelinating disease is maintained or has worsened in the subject.

Accordingly, some aspects of the present disclosure are directed to methods of treating a demyelinating disease in a subject in need thereof, comprising assessing the subject's performance in a test for assessing one or more symptoms associated with a demyelinating disease, wherein the subject received a treatment comprising a PDGFRα inhibitor described herein prior to the assessing. In some aspects, if the subject's performance in the test is improved as compared to a reference performance (e.g., a corresponding subject's performance in the test where the subject was not treated with a PDGFRα inhibitor described herein and/or the subject's performance prior to the treatment with a PDGFRα inhibitor), the treatment comprising a PDGFRα inhibitor can be maintained in the subject (e.g., the subject receives one or more additional administration of the PDGFRα inhibitor at the same dose and/or dosing interval). In some aspects, where the subject exhibits improved performance, the treatment comprising a PDGFRα inhibitor is reduced (e.g., the subject receives one or more additional administration of the PDGFRα inhibitor but at a lower dose and/or longer dosing interval) or stopped. In some aspects, if the subject's performance is comparable or reduced as compared to a reference performance, (e.g., a corresponding subject's performance in the test where the subject was not treated with a PDGFRα inhibitor described herein and/or the subject's performance prior to the treatment with a PDGFRα inhibitor), the treatment comprising a PDGFRα inhibitor is adjusted such that the subject receives one or more administrations of the PDGFRα inhibitor at a higher dose and/or shorter dosing interval.

Some aspects of the present disclosure are directed to methods of improving a subject's performance in a test for assessing one or more symptoms associated with a demyelinating disease, comprising administering to the subject any of the PDGFRα inhibitors described herein. In some aspects, after the administration, the subject's performance in the test is increased as compared to a reference performance (e.g., a corresponding subject's performance in the test where the subject was not treated with a PDGFRα inhibitor described herein and/or the subject's performance prior to the treatment with a PDGFRα inhibitor). In some aspects, the test is selected from a visual evoked potential (VEP) test, a multifocal visual evoked potential (mfVEP) test, a low contrast visual acuity (LC-VA) test, a magnetic resonance imaging (MRI) (e.g., magnetic transfer resonance, myelin water fraction (MWF), and quantitative susceptibility mapping (QSM)), an electromyography (EMG), a nerve conduction velocity (NCV) test, an Extended Disability Status Scale (EDSS), a timed walk test (e.g., timed 25-foot walk), a Nine-Hole Peg Test (9HPT), an ocular coherence tomograph (OCT), a quality of life measure test (e.g., Multiple Sclerosis Quality of Life-54 and Vision-Related Quality of Life), cognitive assessment (e.g., Montreal Cognitive Assessment), or combinations thereof. In some aspects, the test is a LC-VA test. In some aspects, the test is a cognitive assessment.

As is apparent from the present disclosure, the introduction of a PDGFRα inhibitor into a subject can be done by any suitable route, including, but not limited to, intratumorally, orally, pulmonarily, intranasally, parenterally (intravenously, intra-arterially, intramuscularly, intraperitoneally, or subcutaneously), rectally, vaginally, intralymphatically, intrathecally, periocularly, cutaneously, intradermally, intraorbitally, intracerebrally, intracranially, intraspinally, intraventricuarly, intracapsularly, or topically. Administration includes self-administration and the administration by another. A suitable route of administration allows the PDGFRα inhibitors described herein to perform its intended function (e.g., inhibit PDGFRα activity and induce OPC differentiation). In some aspects, a suitable route of administration includes that which would allow a PDGFRα inhibitor to reach the CNS or any other sites where OPCs are present. Non-limiting examples of such routes include intranasal delivery, intrathecal administration, intracranial administration, and combinations thereof. In some aspects, a PDGFRα inhibitor described herein is administered orally. In some aspects, a PDGFRα inhibitor of the present disclosure is administered intravenously. In some aspects, a PDGFRα inhibitor can be administered both orally and intravenously.

In any of the methods provided herein where a PDGFRα inhibitor is administered to a subject, the PDGFRα inhibitor can be administered to the subject using any suitable dosing schedule. In some aspects, the PDGFRα inhibitor is administered to the subject once. In some aspects, multiple doses of the PDGFRα inhibitor are administered to the subject. In some aspects, a PDGFRα inhibitor described herein is administered to a subject according to an intermittent dosing schedule. As used herein, the term “intermittent dosing schedule” (and any variants thereof) refers to a dosing schedule in which the therapeutic agent (e.g., PDGFRα inhibitor) is administered non-continuously (i.e., an intervening period exists between doses). An intermittent dosing schedule useful for the present disclosure can encompass any discontinuous administration regimen that provides a therapeutically effective amount of a PDGFRα inhibitor to a subject in need thereof. Intermittent dosing regimens can use equivalent, lower, or higher doses of a PDGFRα inhibitor than would be used in continuous dosing regimens. Advantages of intermittent dose administration include, but are not limited to, improved safety, decreased toxicity (e.g., decreased weight loss), acceptable levels of ADME criteria, acceptable levels of undesirable effects on organ systems such as heart, pulmonary, hepatic, reproductive (for example, ovarian or testicular) or gastrointestinal, increased exposure, increased efficacy, and/or increased subject compliance. These advantages can be realized when the PDGFRα inhibitor is administered as a single agent and/or when administered in combination with one or more additional therapeutic agents, e.g., standard of care. When administered in combination with one or more additional therapeutic agents, in some aspects, the dosing regimen for the PDGFRα inhibitor (e.g., intermittent dosing) and the additional therapeutic agents are independent of each other. For example, in some aspects, the PDGFRα inhibitor is administered using a first dosing regimen and the additional therapeutic agent is administered using a second dosing regimen, wherein the first and second dosing regimens are different.

In some aspects, the intermittent dosing schedule comprises administering the PDGFRα inhibitor to the subject every other day. In some aspects, the PDGFRα inhibitor is administered to the subject every three days. In some aspects, the PDGFRα inhibitor is administered to the subject every four days. In some aspects, the PDGFRα inhibitor is administered to the subject every five days. In some aspects, the PDGFRα inhibitor is administered to the subject every six days. In some aspects, the PDGFRα inhibitor is administered to the subject once a week. In some aspects, the PDGFRα inhibitor is administered to the subject once every eight days. In some aspects, the PDGFRα inhibitor is administered to the subject once every nine days. In some aspects, the PDGFRα inhibitor is administered to the subject every 10 days. In some aspects, the PDGFRα inhibitor is administered to the subject every 11 days. In some aspects, the PDGFRα inhibitor is administered to the subject every 12 days. In some aspects, the PDGFRα inhibitor is administered to the subject every 13 days. In some aspects, the PDGFRα inhibitor is administered to the subject once every two weeks. In some aspects, the PDGFRα inhibitor is administered to the subject once every three weeks. In some aspects, the PDGFRα inhibitor is administered to the subject once a month. In some aspects, the PDGFRα inhibitor is administered to the subject once every five weeks. In some aspects, the PDGFRα inhibitor is administered to the subject once every six weeks, In some aspects, the PDGFRα inhibitor is administered to the subject once every seven weeks. In some aspects, the PDGFRα inhibitor is administered to the subject once every two months. In some aspects, the PDGFRα inhibitor is administered to the subject once every nine weeks. In some aspects, the PDGFRα inhibitor is administered to the subject once every 10 weeks. In some aspects, the PDGFRα inhibitor is administered to the subject once every 11 weeks. In some aspects, the PDGFRα inhibitor is administered to the subject once every three months. In some aspects, the PDGFRα inhibitor is administered to the subject once every four months. In some aspects, the PDGFRα inhibitor is administered to the subject once every five months. In some aspects, the PDGFRα inhibitor is administered to the subject once every six months. In some aspects, the PDGFRα inhibitor is administered to the subject once every twelve months.

In some aspects, the intermittent dosing schedule comprises administering to the subject a first dose and a second dose of a PDGFRα inhibitor, wherein the second dose is administered at least one day after administering the first dose. In some aspects, the second dose is administered at least two days after administering the first dose. In some aspects, the second dose is administered at least three days after administering the first dose. In some aspects, the second dose is administered at least four days after administering the first dose. In some aspects, the second dose is administered at least five days after administering the first dose. In some aspects, the second dose is administered at least six days after administering the first dose. In some aspects, the second dose is administered at least seven days after administering the first dose. In some aspects, the second dose is administered at least eight days after administering the first dose. In some aspects, the second dose is administered at least nine days after administering the first dose. In some aspects, the second dose is administered at least 10 days after administering the first dose. In some aspects, the second dose is administered at least 11 days after administering the first dose. In some aspects, the second dose is administered at least 12 days after administering the first dose. In some aspects, the second dose is administered at least 13 days after administering the first dose. In some aspects, the second dose is administered at least two weeks after administering the first dose. In some aspects, the second dose is administered at least three weeks after administering the first dose. In some aspects, the second dose is administered at least one month after administering the first dose. In some aspects, the second dose is administered at least two months after administering the first dose. In some aspects, the second dose is administered at least three months after administering the first dose. In some aspects, the second dose is administered at least four months after administering the first dose. In some aspects, the second dose is administered at least five months after administering the first dose. In some aspects, the second dose is administered at least six months after administering the first dose. In some aspects, the second dose is administered at least 12 months after administering the first dose.

Not to be bound by any one theory, in some aspects, the use of an intermittent dosing schedule allows the OPC population of the treated subject (i.e., subject who previously received an administration of the PDGFRα inhibitor) to sufficiently recover prior to the next dose of the PDGFRα inhibitor. As described herein, OPCs are capable of self-renewal and therefore, under a steady-state condition (i.e., no inhibition of PDGFRα activity), the OPC population of a subject is continuously replenished and maintained. As described and demonstrated herein, when a PDGFRα inhibitor described herein is administered to a subject, the PDGFRα inhibitor induces the differentiation of the OPCs within the subject into oligodendrocytes. In some aspects, this can result in a decrease of the OPC population within the subject, and therefore, an immediate or rapid subsequent administration of a PDGFRα inhibitor would have diminished therapeutic activity (e.g., reduced further generation of oligodendrocytes). Accordingly, in some aspects, an intermittent dosing schedule useful for the present disclosure comprises administering a first dose of a PDGFRα inhibitor and a second dose of a PDGFRα inhibitor, wherein the second dose of the PDGFRα inhibitor is administered to the subject after the OPC population of the subject has sufficiently recovered from the effects of the first dose of the PDGFRα inhibitor. When referring to an OPC population (e.g., of a subject treated with a PDGFRα inhibitor), the expression “sufficiently recovered” means that the number of OPCs within the subject has increased, such that an additional administration of the PDGFRα inhibitor is capable of resulting in additional generation of oligodendrocytes. In some aspects, an OPC population has sufficiently recovered is comparable to that of a reference subject, wherein the reference subject comprises (i) the subject prior to the initial administration of the PDGFRα inhibitor, (ii) a corresponding, normal healthy subject (i.e., does not suffer from a demyelinating disease) who has not received an administration of the PDGFRα inhibitor, or (iii) both (i) and (ii). In some aspects, the number of OPCs within an OPC population that has sufficiently recovered is at least 10%, at least 20%, at least 30%, at least 40%, at least 50%, at least 60%, at least 70%, at least 80%, at least 90%, or 100% of the number of OPCs within the OPC population of the reference subject.

Accordingly, where a method provided herein comprises administering a first dose and a second dose of a PDGFRα inhibitor to a subject (e.g., to treat a demyelinating disease), in some aspects, a first dose of the PDGFRα inhibitor is administered to a subject and then the subject's OPC population is assessed, wherein if the subject's OPC population has sufficiently recovered as compared to a reference subject (e.g., as described above), a second dose of the PDGFRα inhibitor is administered to the subject. In some aspects, a second dose of the PDGFRα inhibitor is administered to the subject if the size of the subject's OPC population (e.g., number of OPCs within the OPC population) is at least at least 10%, at least 20%, at least 30%, at least 40%, at least 50%, at least 60%, at least 70%, at least 80%, at least 90%, or 100% of the size of the reference subject's OPC population. In some aspects, where an additional dose of the PDGFRα inhibitor is administered to the subject, the additional dose is administered to the subject at the same dosing interval as that used for the first and second doses of the PDGFRα inhibitor. In some aspects, where an additional dose of the PDGFRα inhibitor is administered to the subject, the method comprises assessing the subject's OPC population again after the second administration, wherein if the subject's OPC population has sufficiently recovered as compared to a reference subject (e.g., as described above), the additional dose of the PDGFRα inhibitor is administered to the subject. In some aspects, the additional dose of the PDGFRα inhibitor is administered to the subject if the size of the subject's OPC population (e.g., number of OPCs within the OPC population) is at least at least 10%, at least 20%, at least 30%, at least 40%, at least 50%, at least 60%, at least 70%, at least 80%, at least 90%, or 100% of the size of the reference subject's OPC population. As further described elsewhere in the present disclosure, in some aspects, the amount of time required for the subject's OPC population to have sufficiently recovered after an initial administration of a PDGFRα inhibitor is the dosing interval of the PDGFRα inhibitor.

Whether a subject's OPC population has sufficiently recovered (and thus, determine a dosing interval for a PDGFRα inhibitor) can be determined using any suitable method known in the art. For example, in some aspects, a cuprizone animal model (e.g., as described in Example 243) can be used. As demonstrated herein, in some aspects, a dosing interval suitable for a PDGFRα inhibitor described herein can be determined by administering a first dose and second dose of the PDGFRα inhibitor to the cuprizone animal model, and then assessing the number of newly generated oligodendrocytes present within the brain of the animals (e.g., by quantifying the number of GPR17+ cells within the brain). If the number of newly generated oligodendrocytes within the brain is the same or increased compared to a reference (e.g., the number of newly generated oligodendrocytes present within the brain of corresponding animal that received a single administration of the PDGFRα inhibitor), then a suitable dosing interval is the time between the administration of the first dose and the second dose of the PDGFRα inhibitor. If the number of newly generated oligodendrocytes within the brain is reduced compared to a reference (e.g., the number of newly generated oligodendrocytes present within the brain of corresponding animal that received a single administration of the PDGFRα inhibitor), then a suitable dosing interval is longer than the time between the administration of the first dose and the second dose of the PDGFRα inhibitor.

In some aspects, a suitable dosing interval for a PDGFRα inhibitor is directly related to the plasma level of the PDGFRα inhibitor. Accordingly, in some aspects, a method of determining a suitable dosing interval for a PDGFRα inhibitor comprises administering a first dose of the PDGFRα inhibitor to a subject and determining the plasma level of the PDGFRα inhibitor in the subject. In some aspects, where the plasma level of the PDGFRα inhibitor is comparable to that of a reference (e.g., a subject who has not previously received a dose of the PDGFRα inhibitor and/or a subject who has previously received a dose of the PDGFRα inhibitor but the subject's OPC population has sufficiently recovered as described herein), a suitable dosing interval is the time between administering the first dose of the PDGFRα inhibitor and the subject's plasma level of the PDGFRα inhibitor being comparable to that of the reference. In some aspects, where the plasma level of the PDGFRα inhibitor is reduced as compared to a reference (e.g., plasma level of the PDGFRα inhibitor in the subject immediately after, e.g., about 4 hours after, the administration of the initial dose of the PDGFRα inhibitor), a suitable dosing interval is the time between administering the first dose of the PDGFRα inhibitor and the subject's plasma level of the PDGFRα inhibitor reaching a reduced level as compared to the reference. In some aspects, the additional dose of the PDGFRα inhibitor is administered to the subject when the subject's plasma level has decreased by at least 10%, at least 20%, at least 30%, at least 40%, at least 50%, at least 60%, at least 70%, at least 80%, at least 90%, or about 100% as compared to the reference.

In some aspects, the plasma level of the PDGFRα inhibitor is related to the half-life of the PDGFRα inhibitor. In some aspects, an intermittent dosing schedule useful for the present disclosure comprises administering two or more doses of a PDGFRα inhibitor at a dosing interval that is longer than the half-life of the PDGFRα inhibitor. In some aspects, the dosing interval is at least 5%, at least 10%, at least 20%, at least 30%, at least 40%, at least 50%, at least 60%, at least 70%, at least 80%, at least 90%, or at least 100% longer than the half-life of the PDGFRα inhibitor. In some aspects, the dosing interval is at least 1.5-fold, at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least about 30-fold, at least 40-fold, or at least 50-fold longer than the half-life of the PDGFRα inhibitor. In some aspects, compared to the half-life of the PDGFRα inhibitor, the dosing interval is at least one day, at least two days, at least three days, at least four days, at least five days, at least six days, at least one week, at least two weeks, at least three weeks, or at least one month longer.

In some aspects, the methods described herein (e.g., treating a demyelinating disease) can comprise administering an additional therapeutic agent to the subject. For examples, in some aspects, the PDGFRα inhibitors described herein are used in conjunction with an immunomodulatory drug. In some aspects, the PDGFRα inhibitors are used alone in the methods provided herein.

As described herein, “immunomodulatory drug” refers to a drug that works by modulating (e.g., increasing and/or decreasing) one or more aspects of an immune response. In some aspects, an immunomodulatory drug useful for the present disclosure is capable of reducing or alleviating the proinflammatory nature of a demyelinating disease (e.g., multiple sclerosis). For instance, in some aspects, the immunomodulatory drug could block the production of proinflammatory mediators, promote the production of anti-inflammatory cytokines (e.g., IL-10 or TGF-β), promote the production of regulatory T cells (T_{regs}), influence B cells, prevent the entry of immune cells into the brain, or any combination thereof. Non-limiting examples of immunomodulatory drugs that are useful for the present disclosure include: interferon beta-1b (BETASERON®, EXTAVIA®), interferon beta-1a (AVONEX®, REBIF®), peginterferon beta-1a (PLEGRIDY®), alemtuzumab (LEMTRADA®), natalizumab (TYSABRI®), ocrelizumab (OCREVUS®), ofatumumab (KESIMPTA®), glatiramer acetate (COPAXONE®, GLATOPA®), teriflunomide (AUBAGIO®), dimethyl fumarate (TECFIDERA®), monomethyl fumarate (BAFIERTAM®), diroximel fumarate (Vumerity®), and combinations thereof.

Where an additional therapeutic agent (e.g., immunomodulatory drug) is administered to a subject, in some aspects, the additional therapeutic agent is administered to the subject prior to the administration of a PDGFRα inhibitor. Accordingly, in some aspects, any of the methods provided herein (e.g., method of treating a demyelinating disease) comprise administering to the subject a PDGFRα inhibitor, wherein the subject had previously received an additional therapeutic agent (e.g., immunomodulatory drug). In some aspects, the additional therapeutic agent is administered after the administration of a PDGFRα inhibitor. In some aspects, the additional therapeutic agent is administered concurrently with the administration of a PDGFRα inhibitor.

In some aspects, to improve delivery to the CNS, a PDGFRα inhibitor described herein (e.g., small molecule or antibody) can be administered to a subject in combination with an agent that assists the delivery of the inhibitor to the CNS (e.g., allows the inhibitor to cross the blood-brain barrier). In some aspects, a PDGFRα inhibitor described herein is administered to a subject in combination with a peptide blood-brain barrier (BBB) shuttle, wherein the peptide BBB shuttle enhances the ability of the PDGFRα inhibitor to cross the blood-brain barrier and reach the CNS. Non-limiting examples of such peptide BBB shuttles are provided in Table 2A (below). See, e.g., Oller-Salvia et al., Chem Soc Rev 45:4690-4707 (2016), and Jafari et al., Expert Opinion on Drug Delivery 16:583-605 (2019).

TABLE 2A
Peptide BBB Shuttle

| SEQ ID | | |
|---|---|---|
| NO | Peptide | Sequence |
| 1 | Angiopep-2 | TFFYGGSRGKRNNFKTEEY-OH |
| 2 | ApoB (3371-3409) | SSVIDALQYKLEGTTRLTRK- |
| | | RGLKLATALSLSNKFVEGS |
| 3 | ApoE (159-167)₂ | (LRKLRKRLL)₂ |
| 4 | Peptide-22 | Ac-C(&)MPRLRGC(&)-NH₂ |
| 5 | THR | THRPPMWSPVWP-NH₂ |
| 6 | THR retro-enantio | pwvpswmpprht-NH₂ |
| 7 | CRT | C(&)RTIGPSVC(&) |
| 8 | Leptin30 | YQQILTSMPSRNVIQISND-LENLRDLLHVL |
| 9 | RVG29 | YTIWMPENPRPGTPCDIFT-NSRGKRASNG-OH |
| 10 | ^{D}CDX | GreirtGraerwsekf-OH |
| 11 | Apamin | C(&₁)NC(&₂)KAPETALC(&₁)-AR-RC(&₂)QQH-NH₂ |
| 12 | MiniAp-4 | [Dap](&)KAPETALD(&) |
| 13 | GSH | γ-L-glutamyl-CG-OH |
| 14 | G23 | HLNILSTLWKYRC |
| 15 | g7 | GFtGFLS(O-β-Glc)-NH₂ |
| 16 | TGN | TGNYKALHPHNG |
| 17 | TAT (47-57) | YGRKKRRQRRR-NH₂ |
| 18 | SynB1 | RGGRLSYSRRRFSTSTGR |
| 19 | Diketopiperazines | &(N-MePhe)-(N-MePhe)Diketo-piperazines |
| 20 | PhPro | (Phenylproline)₄-NH₂ |

Nomenclature for cyclic peptides (&) is adapted to the 3-letter amino acid code from the one described by Spengler et al. Pept. Res., 2005, 65, 550-555

[Dap] stands for diaminopropionic acid.

The disclosure also provides the following particular embodiments.
Embodiment I. A compound of Formula I:

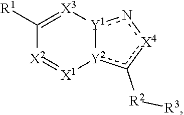

or a pharmaceutically acceptable salt or solvate thereof, wherein:
   [symbol] indicates a single bond or a double bond such that all valences are satisfied;
   X¹, X², X³, and X⁴ are selected from N and CR^{a}, with the proviso that not more than two of X¹, X², X³, and X⁴ are N;
   one of Y¹ and Y² is N and the other of Y¹ and Y² is C;
   each R^{a} is independently selected from H, halo, C₁-C₄alkyl, and alkoxy;
R¹ is selected from C₃-C₈cycloalkyl, 3-8 membered heterocyclyl, heteroaryl, aryl, and C₁-C₈alkoxy;
R² is selected from cycloalkyl, cycloalkenyl, alkyl, oxoalkylamino, aminoalkylamino, oxoalkyloxo, amino, oxo, heterocyclyl, heteroaryl, aminoheterocyclyl, and aminoalkylamino, all of which can be optionally substituted with one, two, three, four, or five substituents selected from H, D, F, oxo, and C₁-C₄alkyl;
R³ is selected from aryl, heteroaryl, C(O)R³¹, C(O)OR³¹, C(O)NR³¹R³², S(O)₂NR³¹R³², cycloalkyl, heterocyclyl, and alkyl, all of which can be optionally substituted with one, two, three, four, or five R³⁰;
   each R³⁰ is independently selected from D, halo, OR³⁰⁰, NR³⁰⁰R³⁰¹, S(O)ᵣR³⁰⁰, and

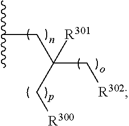

each R³⁰⁰ is independently selected from C₁-C₆alkyl, C₃-C₇cycloalkyl, aryl, heteroaryl, heterocyclyl, and heterocyclylaryl, all of which can be optionally substituted with one, two, three, four, or five substituents selected from D, halo, and C₁-C₄alkyl;
each R³⁰¹ is independently selected from H and C₁-C₄alkyl;
each R³⁰² is independently selected from H, F, hydroxyl, amino, and C₁-C₄alkyoxy;
n, o, and p are each independently selected from 0, 1, 2, 3, and 4;
each R³¹ is independently selected from C₁-C₈alkyl, arylC₁-C₄alkyl, heteroarylC₁-C₄alkyl, heterocyclyl, heterocyclylC₁-C₄alkyl, cycloalkyl, and cycloalkylC₁-C₄alkyl; and
   each R³² is independently selected from H and C₁-C₄alkyl.
Embodiment II. The compound of Embodiment I, or a pharmaceutically acceptable salt or solvate thereof, wherein Y¹ is N and Y² is C.
Embodiment III. The compound of Embodiment I, or a pharmaceutically acceptable salt or solvate thereof, wherein Y¹ is C and Y² is N.
Embodiment IV. The compound of any one of Embodiments I to III, or a pharmaceutically acceptable salt or solvate thereof, wherein X¹ is N, X² is CR^{a}, X³ is CR^{a}, and X⁴ is CR^{a}.
Embodiment V. The compound of any one of Embodiments I to III, or a pharmaceutically acceptable salt or solvate thereof, wherein X¹ is CR^{a}, X² is N, X³ is CR^{a}, and X⁴ is CR^{a}.
Embodiment VI. The compound of any one of Embodiments I to III, or a pharmaceutically acceptable salt or solvate thereof, wherein X¹ is CR^{a}, X² is CR^{a}, X³ is N, and X⁴ is CR^{a}.
Embodiment VII. The compound of any one of Embodiments I to III, or a pharmaceutically acceptable salt or solvate thereof, wherein X¹ is CR^{a}, X² is CR^{a}, X³ is CR^{a}, and X⁴ is N.
Embodiment VIII. The compound of any one of Embodiments I to III, or a pharmaceutically acceptable salt or solvate thereof, wherein X¹ is CR^{a}, X² is CR^{a}, X³ is CR^{a}, and X⁴ is CR^{a}.
Embodiment IX. The compound of any one of Embodiments I to VIII, or a pharmaceutically acceptable salt or solvate thereof, wherein R¹ is 5- or 6-membered heteroaryl.
Embodiment X. The compound of any one of Embodiments I to XIX, or a pharmaceutically acceptable salt or solvate thereof, wherein R¹ is an optionally substituted pyrazolyl.
Embodiment XI. The compound of any one of Embodiments I to X, or a pharmaceutically acceptable salt or solvate thereof, wherein:
R¹ is selected from:

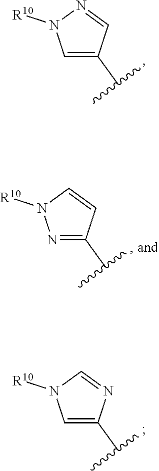

and
R¹⁰ is selected from H, C₁-C₄alkyl, C₁-C₄alkoxy, aminoC₁-C₄alkyl, hydroxyC₁-C₄alkyl, and C₁-C₄alkylsulfonyl.
Embodiment XII. The compound of Embodiment XI, or a pharmaceutically acceptable salt or solvate thereof, wherein R¹ is

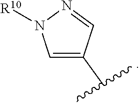

Embodiment XIII. The compound of Embodiment XI or XII, or a pharmaceutically acceptable salt or solvate thereof, wherein R¹⁰ is CH₃.
Embodiment XIV. The compound of any one of Embodiments I to XIII, or a pharmaceutically acceptable salt or solvate thereof, wherein R² is heterocyclyl.
Embodiment XV. The compound of any one of Embodiments I to XIV or a pharmaceutically acceptable salt or solvate thereof, wherein R² is selected from:

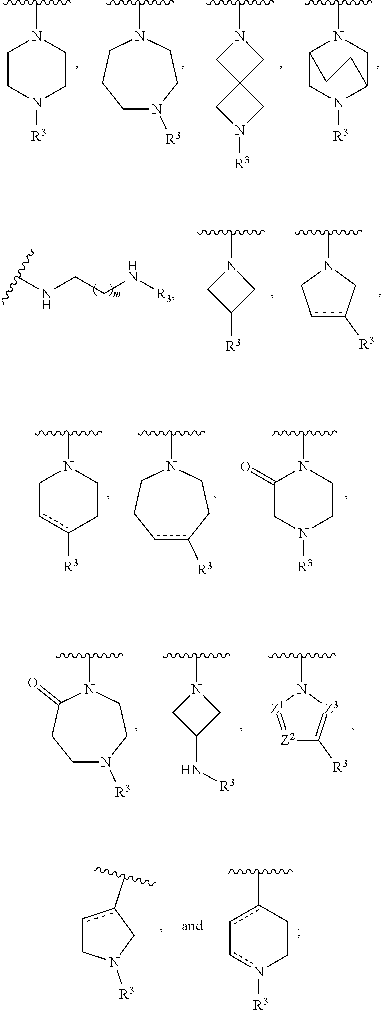

[symbol] indicates a single bond or a double bond such that all valences are satisfied;
m is selected from 0, 1, 2, 3, 4, 5, and 6; and
Z¹, Z², and Z³ are selected from N and CR^{a}.
Embodiment XVI. The compound of Embodiment XV, or a pharmaceutically acceptable salt or solvate thereof, wherein R² is selected from:

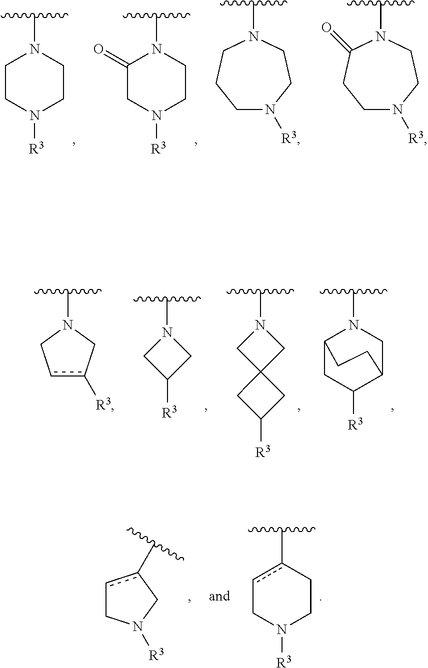

Embodiment XVII. The compound of any one of Embodiments I to XVI, or a pharmaceutically acceptable salt or solvate thereof, having Formula II:

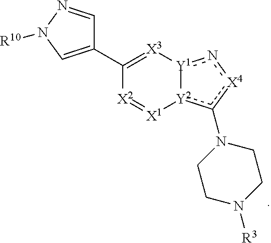

Embodiment XVIII. The compound of any one of Embodiments I to XVII, or a pharmaceutically acceptable salt or solvate thereof, wherein R³ is heteroaryl.
Embodiment XIX. The compound of any one of Embodiments I to XVIII, or a pharmaceutically acceptable salt or solvate thereof, wherein R³ is selected from:

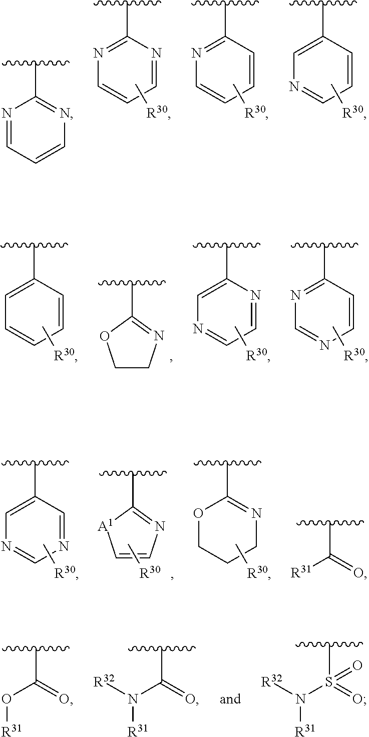

and
wherein A¹ is selected from O, S, and N.
Embodiment XX. The compound of Embodiment XIX, or a pharmaceutically acceptable salt or solvate thereof, wherein R³ is selected from:

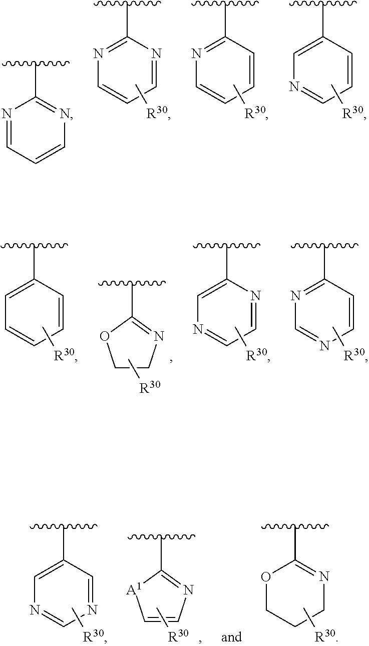

Embodiment XXI. The compound of any of Embodiments I to XX, or a pharmaceutically acceptable salt or solvate thereof, wherein R³⁰ is:

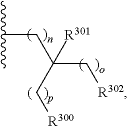

wherein n=0, o=0, and p=0.
Embodiment XXII. The compound of Embodiment XXI, or a pharmaceutically acceptable salt or solvate thereof, wherein R³⁰⁰ is selected from:

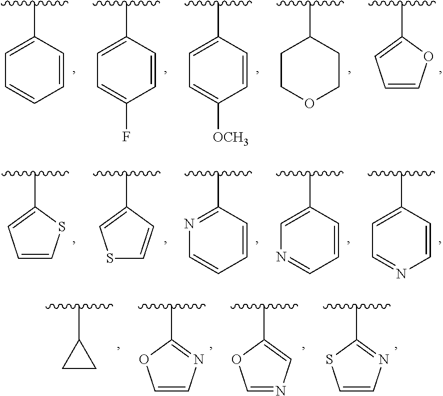

### and H.

Embodiment XXIII. The compound of Embodiment XXII, or a pharmaceutically acceptable salt or solvate thereof, wherein R³⁰⁰ is selected from:

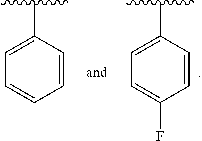 [SMILES: *c1ccccc1 ; *c1ccc(F)cc1]

Embodiment XXIV. The compound of any one of Embodiments XXI to XXIII, or a pharmaceutically acceptable salt or solvate thereof, having Formula III:

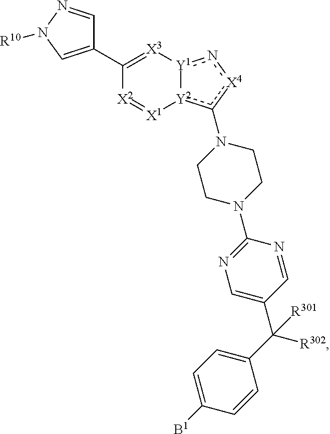

wherein B¹ is H or F.
Embodiment XXV. The compound of any one of Embodiments I to XXIV, or a pharmaceutically acceptable salt or solvate thereof, wherein R³⁰¹ is selected from H and CH₃.
Embodiment XXVI. The compound of any one of Embodiments I to XXV, or a pharmaceutically acceptable salt or solvate thereof, wherein R³⁰² is selected from H and CH₃.
Embodiment XXVII. The compound of any one of Embodiments I to XXVI, or a pharmaceutically acceptable salt or solvate thereof, wherein R³⁰ is benzyl.
Embodiment XXVIII. The compound of any of Embodiments I to XVII, or a pharmaceutically acceptable salt or solvate thereof, wherein R³ is selected from:

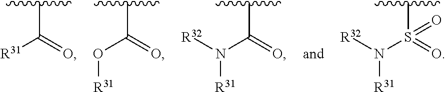

Embodiment XXIX. The compound of Embodiment XXVIII, or a pharmaceutically acceptable salt or solvate thereof, wherein R³ is selected from:

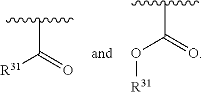

Embodiment XXX. The compound of Embodiments XXVIII or XXIX, or a pharmaceutically acceptable salt or solvate thereof, having Formula IV:

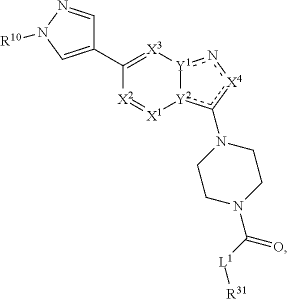

wherein L¹ is a bond or O.
Embodiment XXXI. The compound of any one of Embodiments XXVIII to XXX, or a pharmaceutically acceptable salt or solvate thereof, wherein R³¹ is selected from:

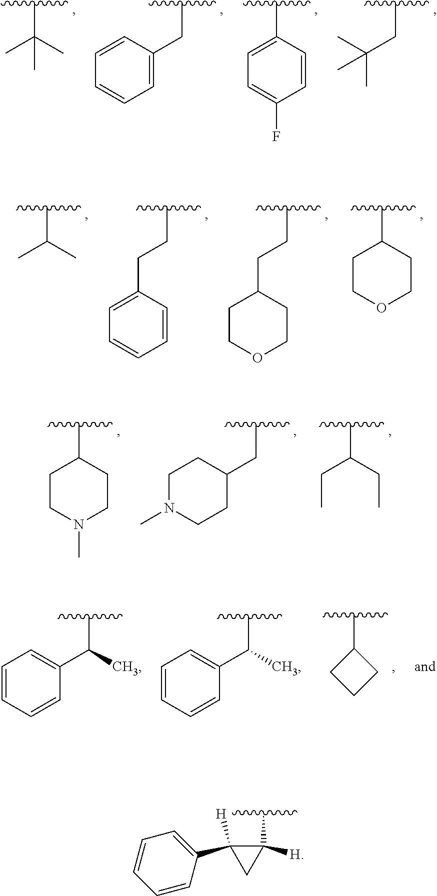

Embodiment XXXII. The compound of Embodiment I, or a pharmaceutically acceptable salt or solvate thereof, selected from any one of the compounds of Table 1.
Embodiment XXXIII. The compound of any one of Embodiments I to XXXII, which is capable of exhibiting one or more of the following properties: (i) promote the differentiation of an OPC into an oligodendrocyte, (ii) promote the expression of a protein associated with oligodendrocyte maturation and/or myelination (e.g., G-protein coupled receptor 17 (GPR17), myelin basic protein (MBP), ASPA, GST-pi, CC1, myelin oligodendrocyte glycoprotein (MOG), oligodendrocyte-specific protein/claudin-11, CNPase, or a combination thereof), (iii) promote the myelination of an axon, (iv) promote the remyelination of a demyelinated axon, (v) inhibit PDGFRα kinase activity, (vi) achieve a brain to plasma ratio of greater than 0.1 when systemically administered to a subject, and (vii) any combination thereof.
Embodiment XXXIV. The compound of Embodiment XXXIII, which is capable of inhibiting PDGFRα kinase activity.
Embodiment XXXV. The compound of Embodiment XXXIV, which is capable of inhibiting PDGFRα kinase activity with an IC₅₀ of less than 500 nM, less than 400 nM, less than 300 nM, less than 200 nM, less than 100 nM, less than 75 nM, less than less than 50 nM, less than 40 nM, less than 30 nM, less than 20 nM, less than 10 nM, or less than 5 nM.
Embodiment XXXVI. The compound of Embodiment XXXV, wherein the IC₅₀ of the PDGFRα inhibitor is determined using an enzymatic PDGFRα kinase assay (e.g., Promega kinase assay described in Example 86).
Embodiment XXXVII. The compound of Embodiment XXXVI, wherein the enzymatic PDGFRα kinase assay comprises 20 ng of purified PDGFRα protein, 150 uM of ATP and 1 ug of substrate, Poly (Glu4Tyr1) in a volume of 15 ul.
Embodiment XXXVIII. A compound that is capable of inhibiting a PDGFRα activity of a cell and further capable of exhibiting one or more of the following properties: (i) promote the differentiation of an OPC into an oligodendrocyte, (ii) promote the expression of a protein associated with oligodendrocyte maturation and/or myelination (e.g., G-protein coupled receptor 17 (GPR17), myelin basic protein (MBP), ASPA, GST-pi, CC1, myelin oligodendrocyte glycoprotein (MOG), oligodendrocyte-specific protein/claudin-11, CNPase, or a combination thereof), (iii) promote the myelination of an axon, (iv) promote the remyelination of a demyelinated axon, (v) inhibit PDGFRα kinase activity, (vii) achieve a brain to plasma ratio of greater than 0.1 when systemically administered to a subject, or (vii) any combination thereof.
Embodiment XXXIX. The compound of Embodiment XXXVIII, which is capable of inhibiting PDGFRα kinase activity with an IC₅₀ of less than 500 nM, less than 400 nM, less than 300 nM, less than 200 nM, less than 100 nM, less than 75 nM, less than less than 50 nM, less than 40 nM, less than 30 nM, less than 20 nM, less than 10 nM, or less than 5 nM.
Embodiment XL. The compound of Embodiment XXXIX, wherein the IC₅₀ of the PDGFRα inhibitor is determined using an enzymatic PDGFRα kinase assay (e.g., Promega kinase assay described in Example 86).
Embodiment XLI. The compound of Embodiment XL, wherein the enzymatic PDGFRα kinase assay comprises 20 ng of purified PDGFRα protein, 150 uM of ATP and 1 μg of substrate, Poly (Glu4Tyr1) in a volume of 15 ul.
Embodiment XLII. The compound of any one of Embodiments XXXVIII to XLI, which comprises a small molecule, an antibody, or both.
Embodiment XLIII. The compound of Embodiment XLII, wherein the small molecule comprises a compound of Formula I:

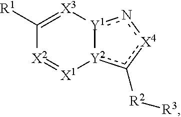

or a pharmaceutically acceptable salt or solvate thereof, wherein:
   [symbol] indicates a single bond or a double bond such that all valences are satisfied;
   X¹, X², X³, and X⁴ are selected from N and CR^{a}, with the proviso that not more than two of X¹, X², X³, and X⁴ are N;
   one of Y¹ and Y² is N and the other of Y¹ and Y² is CR^{a};
   each R^{a} is independently selected from H, halo, C₁-C₄alkyl, and alkoxy;
   R¹ is selected from C₄-C₇cycloalkyl, 4-7 membered heterocyclyl, heteroaryl, aryl, and C₁-C₈alkoxy;
   R² is selected from heterocyclyl, heteroaryl, aminoheterocyclyl, and aminoalkylamino, all of which can be optionally substituted with one, two, three, four, or five substituents selected from H, D, oxo, and C₁-C₄alkyl;
   R³ is selected from aryl, heteroaryl, C(O)R³¹, C(O)OR³¹, C(O)NR³¹R³², S(O)₂NR³¹R³², cycloalkyl, and alkyl, all of which can be optionally substituted with one, two, three, four, or five R³⁰;
   each R³⁰ is independently selected from D, halo, OR³⁰⁰, NR³⁰⁰R³⁰¹, S(O)ᵣR³⁰⁰, and

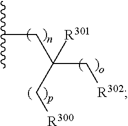

each R³⁰⁰ is independently selected from C₁-C₆alkyl, C₃-C₇cycloalkyl, aryl, heteroaryl, heterocyclyl, and heterocyclylaryl, all of which can be optionally substituted with one, two, three, four, or five substituents selected from D, halo, and C₁-C₄alkyl;
each R³⁰¹ is independently selected from H and C₁-C₄alkyl;
each R³⁰² is independently selected from H, OH, and NH₂;
n, o, and p are each independently selected from 0, 1, 2, 3, and 4;
each R³¹ is independently selected from C₁-C₈alkyl, arylC₁-C₄alkyl, heteroarylC₁-C₄alkyl, heterocyclyl, heterocyclylC₁-C₄alkyl, cycloalkyl, and cycloalkylC₁-C₄alkyl; and
each R³² is independently selected from H and C₁-C₄alkyl.
Embodiment XLIV. The compound of Embodiment XLIII, wherein the compound, or a pharmaceutically acceptable salt or solvate thereof, has Formula II:

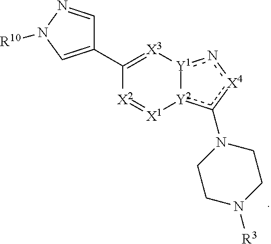

Embodiment XLV. The compound of Embodiment XLIII or XLIV, wherein the compound, or a pharmaceutically acceptable salt or solvate thereof, has Formula III:

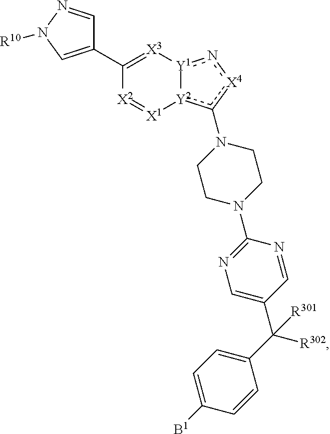

wherein B¹ is H or F.
Embodiment XLVI. The compound of any one of Embodiments XLIII to XLV, wherein the compound, or a pharmaceutically acceptable salt or solvate thereof, has Formula IV:

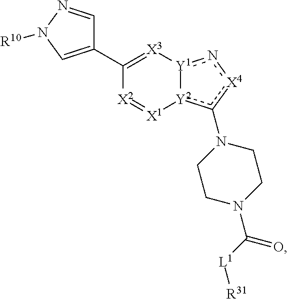

wherein L¹ is a bond or O.
Embodiment XL VII. The compound of any one of Embodiments XLIII to XL VI, which does not comprise any of the following compounds: anlotinib HCl, avapritinib, axitinib, bemcentinib, cediranib, CP-673451, dovitinib, ENMD-2076, foretinib, JNJ=10198409, JNJ-28312141, K252a, linifanib (ABT-869), masitinib, motesanib (AMG706), nintedanib, ON123300, pexidartinib (PLX3397), R81, RO4396686, seralutinib, TAK-593, tamatinib (R-406), tandutinib, telatinib, pazopanib, MK 2461, imatinib, sorafenib, or combinations thereof.
Embodiment XL VIII. A pharmaceutical composition comprising the compound of any one of Embodiments I to XLVII, or a pharmaceutically acceptable salt or solvate thereof, and a pharmaceutically acceptable excipient.
Embodiment XLIX. A kit comprising the compound of any one of Embodiments I to XLVII, or a pharmaceutically acceptable salt or solvate thereof, and instructions for use.
Embodiment L. A method of producing a PDGFRα inhibitor comprising synthesizing the compound of any one of Embodiments I to XL VII.
Embodiment LI. A method of treating a demyelinating disease in a subject in need thereof comprising administering to the subject an effective amount of the compound of any one of Embodiments I to XLVII or the pharmaceutical composition of Embodiment XL VIII.
Embodiment LII. The method of Embodiment LI, wherein the demyelinating disease comprises an acute disseminated encephalomyelitis (ADEM), acute hemorrhagic leukoencephalitis, acute transverse myelitis, adrenoleukodystrophy, adrenomyeloneuropathy, Alexander disease, Alzheimer's disease, aminoacidurias, amyotrophic lateral sclerosis, anti-MAG peripheral neuropathy, anti-MOG associated spectrum, Balo concentric sclerosis, brain injury, CAMFAK Syndrome, Canavan disease, carbon monoxide toxicity, central pontine myelinolysis, cerebral hypoxia, cerebral ischemia, Charcot-Marie-Tooth disease, chronic inflammatory demyelinating polyneuropathy, chronic traumatic encephalopathy, clinically isolated syndrome (CIS), congenital cataract, copper deficiency associated condition, delayed post-hypoxic leukoencephalopathy, diffuse cerebral sclerosis of Schilder, diffuse myelinoclastic sclerosis, extrapontine myelinolysis Gaucher disease, Guillain-Barré syndrome, hereditary neuropathy, hereditary neuropathy with liability to pressure palsy, HTLV-1-associated myelopathy, Hurler syndrome, hypomyelination, hypoxic brain injury, Krabbe disease, Leber hereditary optic atrophy and related mitochondrial disorders, leukodystrophic disorders, Marchiafava-Bignami disease, metachromatic leukodystrophy, multiple sclerosis (e.g., primary progressive multiple sclerosis (PPMS), relapsing-remitting multiple sclerosis (RRMS), secondary progressive multiple sclerosis (SPMS), progressive relapsing multiple sclerosis, Marburg multiple sclerosis, tumefactive multiple sclerosis, and optic-spinal multiple sclerosis), multiple system atrophy, myelinoclastic disorders, myelopathy, nerve injury, neuromyelitis optica (NMO), Niemann-Pick disease, optic neuropathy, optic neuritis (e.g., acute optic neuritis and chronic relapsing inflammatory optic neuritis (CRION)), osmotic demyelination syndrome, Parkinson's disease, Pelizaeus-Merzbacher disease, peripheral neuropathy, phenylketonuria, progressive inflammatory neuropathy, progressive multifocal leukoencephalopathy, progressive subcortical ischemic demyelination, reperfusion injury, Schilder disease, solitary sclerosis, spinal cord injury, subacute sclerosing panencephalitis, Tabes dorsalis, Tay-Sachs disease, traumatic brain injury, tropical spastic paraparesis, vitamin B¹² deficiency, or a combination thereof.
Embodiment LIII. The method of Embodiment LI or LII, wherein the demyelinating disease is characterized by demyelination of one or more cells within the CNS of the subject.
Embodiment LIV. The method of any one of Embodiments LI to LIII, wherein the demyelinating disease is a multiple sclerosis.
Embodiment LV. The method of Embodiment LIV, wherein the multiple sclerosis comprises a clinically isolated syndrome (“CIS”), relapsing-remitting MS (“RRMS”), secondary progressive MS (“SPMS”), primary progressive MS (“PPMS”), or a combination thereof.
Embodiment LVI. The method of any one of Embodiments LI to LIII, wherein the demyelinating disease is an optic neuritis.
Embodiment LVII. The method of any one of Embodiments LI to LVI, wherein treating the demyelinating disease comprises reducing one or more symptoms associated with the demyelinating disease.
Embodiment LVIII. The method of Embodiment LVII wherein the one or more symptoms comprise fatigue, dizziness, malaise, elevated fever and high body temperature, extreme sensitivity to cold in the hands and feet, weakness and stiffness in muscles and joints, weight changes, digestive or gastrointestinal problems, low blood pressure, high blood pressure, irritability, anxiety, depression, blurred vision, double vision, ataxia, clonus, spasms, dysarthria, weakness, clumsiness, hand paralysis, hemiparesis, genital anesthesia, sexual dysfunction, incoordination, paresthesias, ocular paralysis, impaired muscle coordination, loss of sensation, tingling, numbness, pain, impaired vision, neurological symptoms, unsteady gait, balance problems, dizziness, spastic paraparesis, incontinence, hearing problems, speech problems, or combinations thereof.
Embodiment LIX. A method of promoting the myelination of an axon in a subject in need thereof, the method comprising administering to the subject an effective amount of the compound of any one of Embodiments I to XL VII or the pharmaceutical composition of Embodiment XL VIII.
Embodiment LX. The method of Embodiment LIX, wherein the myelination of an axon can be determined by visualizing and/or quantifying the expression of one or more of the following markers: myelin basic protein (MBP) Myelin Oligodendrocyte Glycoprotein (MOG), Oligodendrocyte Specific Protein/Claudin-11, CNPase, or any combination thereof.
Embodiment LXI. A method of promoting the remyelination of a demyelinated axon in a subject in need thereof, the method comprising administering to the subject an effective amount of the compound of any one of Embodiments I to XLVII or the pharmaceutical composition of Embodiment XLVIII.
Embodiment LXII. The method of Embodiment LXI, wherein the remyelination of a demyelinated axon can be determined by visualizing and/or quantifying the expression of one or more of the following markers: myelin basic protein (MBP) Myelin Oligodendrocyte Glycoprotein (MOG), Oligodendrocyte Specific Protein/Claudin-11, CNPase, or any combination thereof.
Embodiment LXIII. A method of reducing the demyelination of a myelinated neuronal axon in a subject in need thereof, the method comprising administering to the subject an effective amount of the compound of any one of Embodiments I to XLVII or the pharmaceutical composition of Embodiment XLVIII.
Embodiment LXIV. The method of Embodiment LXIII, wherein the reduction in the demyelination of a myelinated neuronal axon can be determined by visualizing and/or quantifying the expression of one or more of the following markers: myelin basic protein (MBP) Myelin Oligodendrocyte Glycoprotein (MOG), Oligodendrocyte Specific Protein/Claudin-11, CNPase, or any combination thereof.
Embodiment LXV. A method of activating an oligodendrocyte progenitor cell (OPC) within the central nervous system (CNS) of a subject in need thereof, the method comprising administering to the subject an effective amount of the compound of any one of Embodiments I to XL VII or the pharmaceutical composition of Embodiment XLVIII.
Embodiment LXVI. A method according to any one of Embodiments LIX to LXV, wherein the subject has, or is at risk of developing a demyelinating disease, for example a disease according to any one of Embodiments LI to LVIII.
Embodiment LXVII. A method according to any one of Embodiments LIX to LXV, wherein the method is a method of treating or preventing a demyelinating disease, for example a disease according to any one of Embodiments LI to L VIII.
Embodiment LXVIII. The method of any one of Embodiments LI to LXVII, wherein the compound or the pharmaceutical composition is administered to the subject once.
Embodiment LXIX. The method of any one of Embodiments LI to LXVII, wherein the compound or the pharmaceutical composition is administered to the subject using intermittent dosing.
Embodiment LXX. The method of Embodiment LXIX, wherein the intermittent dosing comprises administering the PDGFRα inhibitor to the subject every other day, every three days, every four days, every five days, every six days, once a week, every eight days, every nine days, every 10 days, every 11 days, every 12 days, every 13 days, once every two weeks, once every three weeks, once a month, once every two months, once every three months, once every four months, once every five months, or once every six months.
Embodiment LXXI. The method of any one of Embodiments LI to LXX, wherein after the administration, the compound or the pharmaceutical composition is capable of achieving a brain to plasma ratio of greater than 0.1, greater than 0.2, greater than 0.3, greater than 0.4, greater than 0.5, greater than 0.6, greater than 0.7, greater than 0.8, greater than 0.9, greater than 1.0, greater than 1.1, greater than 1.2, greater than 1.3, greater than 1.4, greater than 1.5, greater than 1.6, greater than 1.7, greater than 1.8, greater than 1.9, or greater than 2.0.
Embodiment LXXII. The method of any one of Embodiments LI to LXXI, which further comprises administering to the subject an additional therapeutic agent.
Embodiment LXXIII. The method of Embodiment LXXII, wherein the additional therapeutic agent comprises a standard care of treatment.
Embodiment LXXIV. The method of Embodiment LXXII or LXXIII, wherein the additional therapeutic agent comprises an immunomodulatory agent.
Embodiment LXXV. The method of Embodiment LXXIV, wherein the additional therapeutic agent is selected from interferon beta-1^{b}, interferon beta-1a, peginterferon beta-1a, alemtuzumab, natalizumab, ocrelizumab, ofatumumab, glatiramer acetate, teriflunomide, dimethyl fumarate, monomethyl fumarate, diroximel fumarate, fingolimod hydrochloride, siponimod fumaric acid, ozanimod hydrochloride, or a pharmaceutically acceptable salt thereof
Embodiment LXXVI. The method of any one of Embodiments LXXII to LXXV, wherein the additional therapeutic agent is administered to the subject prior to, concurrently, or after the administration of the compound or the pharmaceutical composition.
Embodiment LXXVII. A method of inducing the differentiation of an oligodendrocyte progenitor cell (OPC) into an oligodendrocyte, the method comprising contacting the OPC with an effective amount of the compound of any one of Embodiments I to XLVII or the pharmaceutical composition of Embodiment XL VIII.
Embodiment LXXVIII. The method of Embodiment LXXVII, wherein the differentiation of the OPC into an oligodendrocyte is measured by determining the expression of GPR17, MBP, ASPA, GST-pi, CC1, myelin oligodendrocyte glycoprotein (MOG), oligodendrocyte-specific protein/claudin-11, CNPase, or a combination thereof.
Embodiment LXXIX. A method of inhibiting PDGFRα activity in a cell, the method comprising contacting the cell with an effective amount of the compound of any one of Embodiments I to XL VII or the pharmaceutical composition of Embodiment XL VIII.
Embodiment LXXX. The method of Embodiment LXXIX, wherein the inhibition of the PDGFRα activity is measured by one or more of the following: an in vitro OPC differentiation assay (e.g., as described in Example 87), a cuprizone model for demyelination (e.g., as described in Example 90), an enzymatic PDGFRα kinase assay (e.g., as described in Example 86), or any combination thereof.
Embodiment LXXXI. The method of any one of Embodiments LXXVII to LXXX, wherein the contacting occurs ex vivo or in vivo.
Embodiment LXXXII. A method of treating a demyelinating disease in a subject in need thereof, the method comprising administering to the subject an effective amount of a PDGFRα inhibitor, wherein the PDGFRα inhibitor is capable of inducing the differentiation of an oligodendrocyte progenitor cell (OPC) into an oligodendrocyte.
Embodiment LXXXIII. The method of Embodiment LXXXII, wherein the differentiation of the OPC into an oligodendrocyte is measured by determining the expression of GPR17, MBP, ASPA, GST-pi, CC1, myelin oligodendrocyte glycoprotein (MOG), oligodendrocyte-specific protein/claudin-11, CNPase, or a combination thereof.
Embodiment LXXXIV. The method of Embodiment LXXXII or LXXXIII, wherein the demyelinating disease comprises an acute disseminated encephalomyelitis (ADEM), acute hemorrhagic leukoencephalitis, acute transverse myelitis, adrenoleukodystrophy, adrenomyeloneuropathy, Alexander disease, Alzheimer's disease, aminoacidurias, amyotrophic lateral sclerosis, anti-MAG peripheral neuropathy, anti-MOG associated spectrum, Balo concentric sclerosis, brain injury, CAMFAK Syndrome, Canavan disease, carbon monoxide toxicity, central pontine myelinolysis, cerebral hypoxia, cerebral ischemia, Charcot-Marie-Tooth disease, chronic inflammatory demyelinating polyneuropathy, chronic traumatic encephalopathy, clinically isolated syndrome (CIS), congenital cataract, copper deficiency associated condition, delayed post-hypoxic leukoencephalopathy, diffuse cerebral sclerosis of Schilder, diffuse myelinoclastic sclerosis, extrapontine myelinolysis Gaucher disease, Guillain-Barré syndrome, hereditary neuropathy, hereditary neuropathy with liability to pressure palsy, HTLV-1-associated myelopathy, Hurler syndrome, hypomyelination, hypoxic brain injury, Krabbe disease, Leber hereditary optic atrophy and related mitochondrial disorders, leukodystrophic disorders, Marchiafava-Bignami disease, metachromatic leukodystrophy, multiple sclerosis (e.g., primary progressive multiple sclerosis (PPMS), relapsing-remitting multiple sclerosis (RRMS), secondary progressive multiple sclerosis (SPMS), progressive relapsing multiple sclerosis, Marburg multiple sclerosis, tumefactive multiple sclerosis, and optic-spinal multiple sclerosis), multiple system atrophy, myelinoclastic disorders, myelopathy, nerve injury, neuromyelitis optica (NMO), Niemann-Pick disease, optic neuropathy, optic neuritis (e.g., acute optic neuritis and chronic relapsing inflammatory optic neuritis (CRION)), osmotic demyelination syndrome, Parkinson's disease, Pelizaeus-Merzbacher disease, peripheral neuropathy, phenylketonuria, progressive inflammatory neuropathy, progressive multifocal leukoencephalopathy, progressive subcortical ischemic demyelination, reperfusion injury, Schilder disease, solitary sclerosis, spinal cord injury, subacute sclerosing panencephalitis, Tabes dorsalis, Tay-Sachs disease, traumatic brain injury, tropical spastic paraparesis, vitamin B12 deficiency, or a combination thereof.
Embodiment LXXXV. The method of any one of Embodiments LXXXII to LXXXIV, wherein the demyelinating disease is characterized by demyelination of one or more cells within the CNS of the subject.
Embodiment LXXXVI. The method of any one of Embodiments LXXXII to LXXXV, wherein the demyelinating disease is a multiple sclerosis.
Embodiment LXXXVII. The method of Embodiment LXXXVI, wherein the multiple sclerosis comprises a clinically isolated syndrome (“CIS”), relapsing-remitting MS (“RRMS”), secondary progressive MS (“SPMS”), primary progressive MS (“PPMS”), or a combination thereof.
Embodiment LXXXVIII. The method of any one of Embodiments LXXXII to LXXXV, wherein the demyelinating disease is an optic neuritis.
Embodiment LXXXIX. The method of any one of Embodiments LXXXII to LXXXVIII, wherein treating the demyelinating disease comprises reducing one or more symptoms associated with the demyelinating disease.
Embodiment XC. The method of Embodiment LXXXIX, wherein the one or more symptoms comprise fatigue, dizziness, malaise, elevated fever and high body temperature, extreme sensitivity to cold in the hands and feet, weakness and stiffness in muscles and joints, weight changes, digestive or gastrointestinal problems, low blood pressure, high blood pressure, irritability, anxiety, depression, blurred vision, double vision, ataxia, clonus, spasms, dysarthria, weakness, clumsiness, hand paralysis, hemiparesis, genital anesthesia, sexual dysfunction, incoordination, paresthesias, ocular paralysis, impaired muscle coordination, loss of sensation, tingling, numbness, pain, impaired vision, neurological symptoms, unsteady gait, balance problems, dizziness, spastic paraparesis, incontinence, hearing problems, speech problems, or combinations thereof.
Embodiment XCI. A method of promoting the myelination of an axon in a subject in need thereof, the method comprising administering to the subject an effective amount of a PDGFRα inhibitor, wherein the PDGFRα inhibitor is capable of inducing the differentiation of an oligodendrocyte progenitor cell (OPC) into an oligodendrocyte.
Embodiment XCII. The method of Embodiment XCI, wherein the myelination of an axon can be determined by visualizing and/or quantifying the expression of one or more of the following markers: myelin basic protein (MBP) Myelin Oligodendrocyte Glycoprotein (MOG), Oligodendrocyte Specific Protein/Claudin-11, CNPase, or any combination thereof.
Embodiment XCIII. A method of promoting the remyelination of a demyelinated axon in a subject in need thereof, the method comprising administering to the subject an effective amount of a PDGFRα inhibitor, wherein the PDGFRα inhibitor is capable of inducing the differentiation of an oligodendrocyte progenitor cell (OPC) into an oligodendrocyte.
Embodiment XCIV. The method of Embodiment XCIII, wherein the remyelination of a demyelinated axon can be determined by visualizing and/or quantifying the expression of one or more of the following markers: myelin basic protein (MBP) Myelin Oligodendrocyte Glycoprotein (MOG), Oligodendrocyte Specific Protein/Claudin-11, CNPase, or any combination thereof.
Embodiment XCV. A method of reducing the demyelination of a myelinated axon in a subject in need thereof, the method comprising administering to the subject an effective amount of a PDGFRα inhibitor, wherein the PDGFRα inhibitor is capable of inducing the differentiation of an oligodendrocyte progenitor cell (OPC) into an oligodendrocyte.
Embodiment XCVI. The method of Embodiment XCV, wherein the reduction in the demyelination of a myelinated neuronal axon can be determined by visualizing and/or quantifying the expression of one or more of the following markers: myelin basic protein (MBP) Myelin Oligodendrocyte Glycoprotein (MOG), Oligodendrocyte Specific Protein/Claudin-11, CNPase, or any combination thereof.
Embodiment XCVII. A method of activating an oligodendrocyte progenitor cell (OPC) within the central nervous system (CNS) of a subject in need thereof, the method comprising administering to the subject an effective amount of a PDGFRα inhibitor, wherein the PDGFRα inhibitor is capable of inducing the differentiation of an oligodendrocyte progenitor cell (OPC) into an oligodendrocyte.
Embodiment XCVIII. The method of any one of Embodiments XCI to XCVII, wherein the PDGFRα inhibitor is administered to the subject once.
Embodiment XCVIX. The method of any one of Embodiments XCI to XCVII, wherein the PDGFRα inhibitor is administered to the subject using intermittent dosing.
Embodiment C. The method of Embodiment XCVIX, wherein the intermittent dosing comprises administering the PDGFRα inhibitor to the subject every other day, every three days, every four days, every five days, every six days, once a week, every eight days, every nine days, every 10 days, every 11 days, every 12 days, every 13 days, once every two weeks, once every three weeks, once a month, once every two months, once every three months, once every four months, once every five months, or once every six months.
Embodiment CI. The method of any one of Embodiments XCI to C, wherein after the administration, the PDGFRα inhibitor is capable of achieving a brain to plasma ratio of greater than 0.1, greater than 0.2, greater than 0.3, greater than 0.4, greater than 0.5, greater than 0.6, greater than 0.7, greater than 0.8, greater than 0.9, greater than 1.0, greater than 1.1, greater than 1.2, greater than 1.3, greater than 1.4, greater than 1.5, greater than 1.6, greater than 1.7, greater than 1.8, greater than 1.9, or greater than 2.0.
Embodiment CII. The method of any one of Embodiments XCI to CI, which further comprises administering to the subject an additional therapeutic agent.
Embodiment CIII. The method of Embodiment CII, wherein the additional therapeutic agent comprises a standard care of treatment.
Embodiment CIV. The method of Embodiment CII or CIII, wherein the additional therapeutic agent comprises an immunomodulatory agent.
Embodiment CV. The method of Embodiment CIV, wherein the additional therapeutic agent is selected from interferon beta-1b, interferon beta-1a, peginterferon beta-1a, alemtuzumab, natalizumab, ocrelizumab, ofatumumab, glatiramer acetate, teriflunomide, dimethyl fumarate, monomethyl fumarate, diroximel fumarate, fingolimod hydrochloride, siponimod fumaric acid, ozanimod hydrochloride, or a pharmaceutically acceptable salt thereof.
Embodiment CVI. The method of any one of Embodiments CII to CV, wherein the additional therapeutic agent is administered to the subject prior to, concurrently, or after the administration of the compound or the pharmaceutical composition.
Embodiment CVII. A method of inducing the differentiation of an oligodendrocyte progenitor cell (OPC) into an oligodendrocyte, the method comprising contacting the OPC with an effective amount of a PDGFRα inhibitor.
Embodiment CVIII. The method of Embodiment CVII, wherein the differentiation of the OPC into an oligodendrocyte is measured by determining the expression of GPR17, MBP, ASPA, GST-pi, CC1, myelin oligodendrocyte glycoprotein (MOG), oligodendrocyte-specific protein/claudin-11, CNPase, or a combination thereof.
Embodiment CVIX. The method of Embodiment CVII or CVIII, wherein the contacting occurs ex vivo or in vivo.
Embodiment CX. The method of any one of Embodiments LXXXII to CIX, wherein the PDGFRα inhibitor comprises a small molecule, an antibody, or both.
Embodiment CXI. The method of Embodiment CX, wherein the PDGFRα inhibitor is a small molecule that is capable of inhibiting the activity of a kinase of PDGFRα.
Embodiment CXII. The method of Embodiment CX, wherein the PDGFRα inhibitor is an antibody that is capable of binding to the extracellular region of PDGFRα.
Embodiment CXIII. The method of any one of Embodiments LXXXII to CXII, wherein the PDGFRα inhibitor is capable of inhibiting the activity of PDGFRα in the subject with an IC₅₀ of less than 500 nM, less than 400 nM, less than 300 nM, less than 200 nM, less than 100 nM, less than 75 nM, less than less than 50 nM, less than 40 nM, less than 30 nM, less than 20 nM, less than 10 nM, or less than 5 nM.
Embodiment CXIV. The method of Embodiment CXIII, wherein the IC₅₀ of the PDGFRα inhibitor is determined using an enzymatic PDGFRα kinase assay (e.g., Promega kinase assay described in Example 86).
Embodiment CXV. The method of Embodiment CXIV, wherein the enzymatic PDGFRα kinase assay comprises 20 ng of purified PDGFRα protein, 150 uM of ATP and 1 μg of substrate, Poly (Glu4Tyr1) in a volume of 15 ul.
Embodiment CXVI. A method of treating a relapsing form of multiple sclerosis in a subject in need thereof, the method comprising administering to the subject an effective amount of the compound of any one of Embodiments I to XLVII or the pharmaceutical composition of Embodiment XLVIII.
Embodiment CXVII. A method of treating a relapsing form of multiple sclerosis in a subject in need thereof, the method comprising administering to the subject an effective amount of a PDGFRα inhibitor, wherein the PDGFRα inhibitor is capable of inducing the differentiation of an oligodendrocyte progenitor cells (OPC) into an oligodendrocyte.
Embodiment CXVIII. The method of Embodiment CXVII, wherein the differentiation of the OPC into an oligodendrocyte is measured by determining the expression of GPR17, MBP, ASPA, GST-pi, CC1, myelin oligodendrocyte glycoprotein (MOG), oligodendrocyte-specific protein/claudin-11, CNPase, or a combination thereof.
Embodiment CXVIX. The method of Embodiment CXVII or CXVIII, wherein the relapsing form of multiple sclerosis comprises a clinically isolated syndrome (“CIS”), relapsing-remitting MS (“RRMS”), secondary progressive MS (“SPMS”), primary progressive MS (“PPMS”), or a combination thereof.
Embodiment CXX. The compound of any one of Embodiments I to XL VII or the pharmaceutical composition of Embodiment XL VIII, for use in a method of one or more of the following: (i) promote the differentiation of an OPC into an oligodendrocyte, (ii) promote the expression of a protein associated with oligodendrocyte maturation and/or myelination (e.g., G-protein coupled receptor 17, myelin basic protein (MBP), ASPA, GST-pi, CC1, myelin oligodendrocyte glycoprotein (MOG), oligodendrocyte-specific protein/claudin-11, CNPase, or a combination thereof), (iii) promote the myelination of an axon, (iv) promote the remyelination of a demyelinated axon, (v) inhibit PDGFRα kinase activity, (vi) achieve a brain to plasma ratio of greater than 0.1 when systemically administered to a subject, and (vii) any combination thereof.
Embodiment CXXI. A compound that is capable of inhibiting a PDGFRα activity of a cell (“PDGFRα inhibitor”), for use in a method of one or more of the following: (i) promote the differentiation of an OPC into an oligodendrocyte, (ii) promote the expression of a protein associated with oligodendrocyte maturation and/or myelination (e.g., G-protein coupled receptor 17 (GPR17), myelin basic protein (MBP), ASPA, GST-pi, CC1, myelin oligodendrocyte glycoprotein (MOG), oligodendrocyte-specific protein/claudin-11, CNPase, or a combination thereof), (iii) promote the myelination of an axon, (iv) promote the remyelination of a demyelinated axon, (v) inhibit PDGFRα kinase activity, (vi) achieve a brain to plasma ratio of greater than 0.1 when systemically administered to a subject, or (vii) any combination thereof.
Embodiment CXXII. A method of treating a demyelinating disease in a subject in need thereof, the method comprising administering to the subject an effective amount of a PDGFRα inhibitor, wherein the PDGFRα inhibitor treats the demyelinating disease by inducing the differentiation of an oligodendrocyte progenitor cell (OPC) into an oligodendrocyte.
Embodiment CXXIII. A method of promoting the myelination of a neuronal axon in a subject in need thereof, the method comprising administering to the subject an effective amount of a PDGFRα inhibitor, wherein the PDGFRα inhibitor promotes the myelination of a neuronal axon by inducing the differentiation of an oligodendrocyte progenitor cell (OPC) into an oligodendrocyte.
Embodiment CXXIV. A method of promoting the remyelination of a demyelinated neuronal axon in a subject in need thereof, the method comprising administering to the subject an effective amount of a PDGFRα inhibitor, wherein the PDGFRα inhibitor promotes the remyelination of the demyelinated neuronal axon by inducing the differentiation of an oligodendrocyte progenitor cell (OPC) into an oligodendrocyte.
Embodiment CXXV. A method of reducing the demyelination of a myelinated neuronal axon in a subject in need thereof, the method comprising administering to the subject an effective amount of a PDGFRα inhibitor, wherein the PDGFRα inhibitor reduces the demyelination of the myelinated neuronal axon by inducing the differentiation of an oligodendrocyte progenitor cell (OPC) into an oligodendrocyte.
Embodiment CXXVI. A method of activating an oligodendrocyte progenitor cell (OPC) within the central nervous system (CNS) of a subject in need thereof, the method comprising administering to the subject an effective amount of a PDGFRα inhibitor, wherein the PDGFRα inhibitor activates the OPC within the CNS by inducing the differentiation of the OPC into an oligodendrocyte.

The specification is most thoroughly understood in light of the teachings of the references cited within the specification. The embodiments within the specification provide an illustration of embodiments and should not be construed to limit the scope. The skilled artisan readily recognizes that many other embodiments are encompassed. All publications and patents cited in this disclosure are incorporated by reference in their entirety. To the extent the material incorporated by reference contradicts or is inconsistent with this specification, the specification will supersede any such material. The citation of any references herein is not an admission that such references are prior art.

Unless otherwise indicated, all numbers expressing quantities of ingredients, reaction conditions, and so forth used in the specification, including claims, are to be understood as being modified in all instances by the term “about.” Accordingly, unless otherwise indicated to the contrary, the numerical parameters are approximations and can vary depending upon the desired properties sought to be obtained. At the very least, and not as an attempt to limit the application of the doctrine of equivalents to the scope of the claims, each numerical parameter should be construed in light of the number of significant digits and ordinary rounding approaches.

The following examples are offered by way of illustration and not by way of limitation. The contents of all references cited throughout this application are expressly incorporated herein by reference.

### EXAMPLES

As further detailed below, the present disclosure demonstrates that compounds that inhibit PDGFRα kinase activity in OPCs can induce oligodendrocyte differentiation (e.g., in vitro and in vivo), and that the effect is likely specific to the inhibition of PDGFRα kinase activity. Furthermore, the present disclosure demonstrates that such compounds can induce remyelination in an animal model of demyelination.

Table 2B below defines the different abbreviations used in the below examples and else wherein the present disclosure.

TABLE 2B
Abbreviations

| Term | Abbreviation |
|---|---|
| 1,1′-Bis(diphenylphosphino)ferrocene | dppf |
| Acetic acid (CH₃CO₂H) | AcOH |
| Acetonitrile (CH₃CN) | MeCN |
| Aqueous | aq. |
| Chloro[2-(di-tert- | tBuXPhos |
| butylphosphino)-2′,4′,6′- | Pd G1 |
| triisopropyl-1,1′-biphenyl][2-(2- | |
| aminoethyl)phenyl)]palladium(II) | |
| Column volume | CV |
| Dichloromethane (CH₂Cl₂) | DCM |
| Diethyl ether ((CH₃CH₂)₂O) | Et₂O |
| Dimethyl sulfoxide ((CH₃)₂SO) | DMSO |
| Electrospray ionization | ESI |
| Ethanol (CH₃CH₂OH) | EtOH |
| Ethyl acetate | EtOAc |
| Ethylene dichloride (1,2- | EDC |
| Dichloroethane) | |
| High performance liquid | HPLC |
| chromatography | |
| Isopropyl alcohol | IPA |
| Liquid chromatograph-mass | LCMS |
| spectrometry | |
| Lithium bis(trimethylsilyl)amide | LiHMDS |
| Mass spectrometry | MS |
| Methanol (CH₃OH) | MeOH |
| Methyl tert-butyl ether | MTBE |
| N,N-Diisopropylethylamine | DIEA |
| N,N-dimethylformamide | DMF |
| N-bromosuccinimide | NBS |
| N-chlorosuccinimide | NCS |
| N-Methyl-2-pyrrolidone | NMP |
| Nuclear magnetic resonance | NMR |
| Palladium on carbon | Pd/C |
| Parts per million | ppm |
| Petroleum ether | PE |
| Room temperature | rt |
| Saturated | sat'd |
| Starting material | SM |
| Tetrahydrofuran | THF |
| Thin-layer chromatography | TLC |
| Trifluoroacetic acid (CF₃CO₂H) | TFA |
| Trimethylamine ((CH₃CH₂)₃N) | Et₃N, TEA |

### Example 1: Analysis of the Effect of PDGFRα Inhibition on Oligodendrocyte Differentiation

According to the present disclosure, it was hypothesized that compounds that can inhibit PDGFRα activity can induce remyelination. To test this, known compounds with PDGFRα activity were identified and tested in vitro for their ability to induce differentiation of OPCs into oligodendrocytes and also the expression of myelin specific proteins. Differentiation was monitored by assessing expression of GPR17, a protein preferentially expressed in newly differentiated, or premyelinating oligodendrocytes. MBP expression was used to assess myelin specific protein expression.

### Example 2: Role of PDGFRα Blockade on OPC Differentiation

To determine the specific role of PDGFRα inhibition in oligodendrocyte differentiation, in vitro experiments were conducted using a selective anti-PDGFRα antibody. The antibody binds to the extracellular region of PDGFRα, and therefore, could inhibit PDGFRα activity by various mechanisms, including, but not limited to, blocking the binding of PDGFRα to its natural ligand and/or blocking receptor dimerization. Anti-PDGFRβ and IgG isotype specific antibodies were used as controls.

### Methods

### OPC Isolation and Seeding:

Cortices from postnatal day (P) 1-3 mice were dissected and collected into ice cold Hank's Balanced Salt Solution (HBSS) containing 26 mM HEPES, 0.3% glucose, and 0.75% sucrose. A homogenous cell suspension of the cortices was made using the Neural Tissue Dissociation Kit (P) (Miltenyi #130-092-628) as per manufacturer's protocol. Oligodendrocyte progenitor cells (OPCs) were isolated from the mixed cortical cell suspension by positive selection using Anti-A2B5 magnetic beads (Miltenyi #130-093-388, as per manufacturer's protocol). Briefly, the cortical cell suspension was centrifuged and washed with BSA buffer (0.5% BSA (bovine serum albumin) in PBS (phosphate buffered saline)) and labeled with A2B5 beads (10 μL beads per 1×10⁷ cells) for 15 minutes at 4° C. Following the incubation, cells were washed once with BSA buffer to remove unlabeled beads. A2B⁵ positive cells were eluted on the column using the autoMACS® (Miltenyi, as per manufacturer's protocol using inbuilt “Possel” program). Eluted cells were further washed twice with OPC growth medium (DMEM SATO base growth culture media (Emery and Dugas, Cold Spring Harboar Protoc 2013(9): 854-68 (September 2013) containing 20 ng/ml of PDGF (Peprotech 100-13A) and 20 ng/ml of fibroblast growth factor (FGF) (R&D Systems #233-FB-010). Cell counts were performed and A2B5+ OPCs were plated in poly-d-lysine (PDL)-coated T75 flasks at a density of 3-4 million cells per flask. Cells were allowed to expand for 3 days at 37° C., 5% CO₂ in OPC growth medium.

### Antibody-Blocking Treatment:

Following three days of expansion (˜60-70% confluency), cells were lifted using TrypLE™ (Thermo 12604013) for 3-5 minutes at 37° C., 5% CO₂. TrypLE was inactivated by adding 10 mL of PDGF-containing OPC growth media and cells were centrifuged at 1400 rpm for 10 minutes. The cell pellet was resuspended in OPC growth medium, cell count was performed, and cells were plated into PDL coated 96 well plates (PE Cell Carrier #6005550) at 10,000 cells per well in 90 μL medium, and incubated at 37° C., 5% CO₂ for 24 hours. Following the 24 hour incubation, cells were treated with 10 ul of a 10× concentration of IgG control (Invitrogen 31154), PDGFRα-blocking antibody (Invitrogen 14-1401-82, clone APA5), or PDGFRB-blocking antibody (Invitrogen 14-1402-82, clone APB5), resulting in a final 1× concentration. Each of the antibodies were used at four different concentrations: 10 μg/mL, 1 μg/mL, 0.1 μg/mL, and 0.01 μg/mL. Following antibody treatment, cells were cultured for 72 hours at 37° C., 5% CO₂ before immunostaining.

### Immunocytochemistry:

Following 72 hours of treatment, cells were fixed with 4% paraformaldehyde for 15 minutes then washed three times in DPBS. Prior to staining, cells were treated with blocking solution for 15 minutes in 10% normal goat serum containing 1% BSA and 0.1% Triton® X-100 in DPBS. Primary antibody (MBP @ 1:2000; Abcam AB7349) was applied to cells overnight at 4° C. in incubation solution containing 1% normal goat serum, 1% BSA, and 0.1% Triton X-100 in DPBS. After primary incubation, cells were washed three times in DPBS and secondary antibody (Goat anti-Rat Alexa Fluor 488, Jackson Immunoresearch 112-545-003, 1:1000;) were applied in incubation solution for two hours at room temperature, protected from light. Following secondary antibody incubation, cells were washed three times with DPBS and nuclei stained with Hoechst (BioRad #1351304).

### Image Analysis and Quantification:

Cells were imaged using ImageXpress® confocal system (Molecular Devices; 5150105) and analyzed using a custom protocol from MetaXpress Custom Module software. The number of MBP+ cells were normalized to the total nuclei count and percent MBP+ cells were reported. Statistical analysis was done using one-way ANOVA with multiple comparisons and post-hoc Tukey test in GraphPad Prism (GraphPad Software).

### Results:

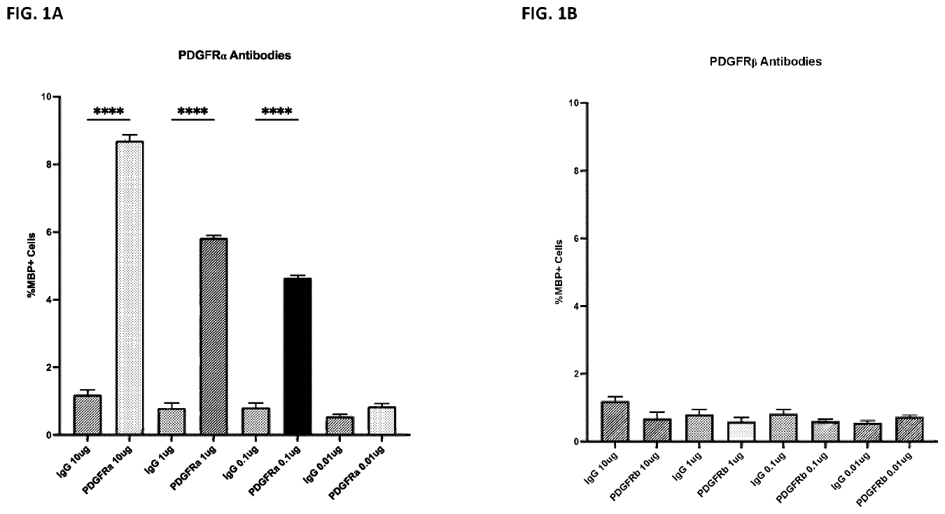

Compared to IgG control, treatment of OPCs with PDGFRα blocking antibody significantly increased the percentage of mature, MBP+ oligodendrocytes after 72 hours (FIG. 1A). Further, the increase in cell differentiation was specific to PDGFRα blockade as OPCs treated with blocking-antibody against PDGFRβ showed no significant increase in MBP+ mature oligodendrocytes (FIG. 1B). These data demonstrate that inhibition of PDGFRα is sufficient to drive OPCs to differentiate into oligodendrocytes and express myelin associated proteins. This further supports the use of PDGFRα inhibitors for inducing remyelination and treating diseases involving demyelination.

### Example 3: Preparation of Compounds for Synthesis of Exemplary PDGFRα Inhibitors

To synthesize exemplary chemical compounds predicted to have antagonistic PDGFRα activity, compounds S1-S29 were first prepared as described below.

### Synthesis of Compound S1 (3-bromo-7-(1-methyl-1H-pyrazol-4-yl)imidazo[1,2-a]pyrimidine)

### 1. Synthesis of Intermediate S1-2

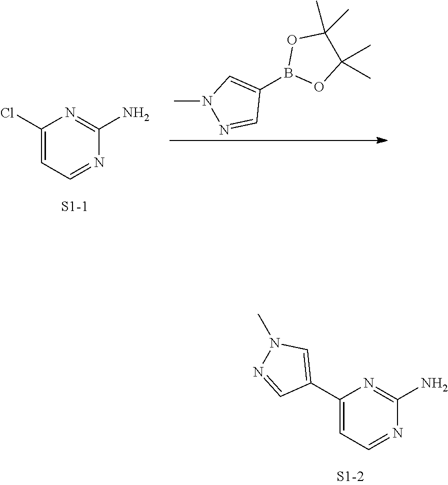

To a mixture of Compound S1-1 (20 g, 154.4 mmol) in dioxane/water (8/1, 480 mL) was added 1-methyl-4-(4,4,5,5-tetramethyl-1,3,2-dioxaborolan-2-yl)-1H-pyrazole (48.2 g, 231.6 mmol), Na₂CO₃ (36.0 g, 339.6 mmol) and Pd(dppf)Cl₂ (3.0 g) under N₂. The mixture was heated to reflux and stirred under N₂ for 2 h. The mixture was cooled to room temperature, filtered. The filtrate was poured into cold water, extracted with DCM/MeOH=10/1 for 10 times, the organic phase was washed with brine, dried over Na₂SO₄, concentrated in vacuum to give crude solid. The crude solid was purified by silica gel chromatography eluted with CH₂Cl₂/MeOH (1:0˜50:1) to give S1-2 (20 g, 74.0%) as a yellow solid.

¹H NMR (DMSO-d₆, 400 MHZ): δ (ppm) 8.26 (s, 1H), 8.15 (d, J=4.8 Hz, 1H), 7.97 (s, 1H), 6.80 (d, J=5.2 Hz, 1H), 6.47 (s, 2H), 3.88 (s, 3H).

### 2. Synthesis of Intermediate Compound S1-3

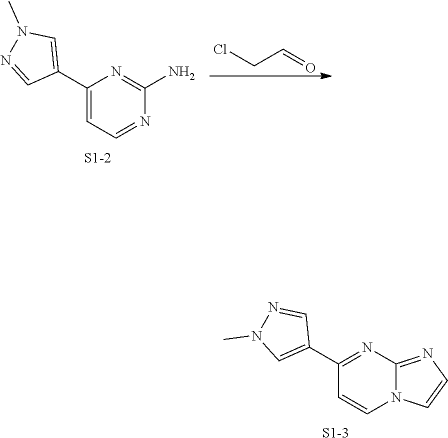

To a mixture of Compound S1-2 (10 g, 57.1 mmol) in dioxane (200 mL) was added NaHCO₃ (28.8 g, 342.6 mmol), 2-chloroacetaldehyde (26.9 g, 342.6 mmol, 40%, aq) under N₂. The mixture was heated to 80° C., and stirred for 2 h. The mixture was cooled to room temperature, concentrated in vacuum to give crude solid. The crude solid was purified by silica gel chromatography to give S1-3 (8 g, crude) as a yellow solid. The crude compound was purified by prep-HPLC to give S1-3 (1.75 g, 15.5%) as a light-yellow solid.

¹H NMR (DMSO-d₆, 400 MHZ): δ (ppm) 8.91 (d, J=5.2 Hz, 1H), 8.88 (s, 1H), 8.50 (d, J=2.4 Hz, 1H), 8.43 (s, 1H), 8.39 (d, J=2.4 Hz, 1H), 7.79 (d, J=4.8 Hz, 1H), 4.01 (s, 3H).

### 3. Synthesis of Compound S1

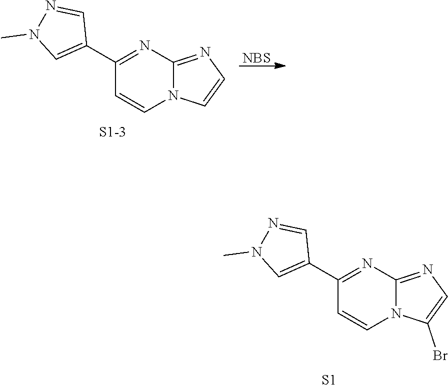

To a mixture of Compound S1-3 (2.0 g, 10.0 mmol) in acetonitrile (60 mL) was added NBS (1.3 g, 7.0 mmol) in portions. The mixture was stirred at 10° C. for 15 min, poured into Na₂S₂O₃(aq), extracted with DCM/MeOH=10/1 for 10 times, the organic phase was washed with Na₂S₂O₃(aq), then brine, dried over Na₂SO₄, concentrated in vacuum to give crude solid. The crude solid was purified by silica gel chromatography to give 3-bromo-7-(1-methyl-1H-pyrazol-4-yl)imidazo[1,2-a]pyrimidine (S1) (0.8 g, 28.0%) as yellow solid.

¹H NMR (DMSO-d₆, 400 MHZ): δ (ppm) 8.53 (d, J=4.4 Hz, 1H), 8.21 (s, 1H), 7.86 (s, 1H), 8.82 (s, 1H), 7.00 (d, J=4.0 Hz, 1H), 3.95 (s, 3H).

### Synthesis of Compound S2

### 1. Synthesis of Intermediate Compound S2-2

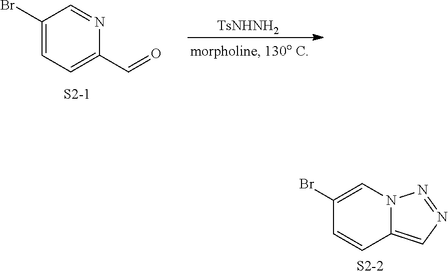

To a mixture of S2-1 (20 g, 107.5 mmol) in MeOH (300 mL) was added TsNHNH₂ (20.9 g, 112.9 mmol). The mixture was stirred at room temperature for 2 h. The mixture was concentrated in vacuum to give crude solid. The residue was dissolved in morpholine (300 mL). The mixture was stirred at 130° C. for 2 h. Then the mixture was poured into cold water, extracted with DCM, the organic phase was washed with brine, dried over Na₂SO₄, concentrated in vacuum to give crude solid. The crude solid was washed with MTBE, dried to give S2-2 (16 g, 74.7%) as a light-yellow solid.

¹H NMR (CDCl₃, 400 MHZ): δ (ppm) 8.92 (s, 1H), 8.08 (s, 1H), 7.65 (d, J=9.6 Hz, 1H), 7.31 (dd, J=1.2, 9.2 Hz, 1H).

### 2. Synthesis of Intermediate Compound S2-3

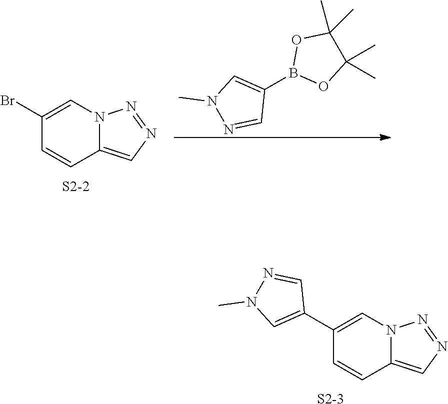

To a mixture of S2-2 (16 g, 80.4 mmol) in dioxane/water (320 mL/64 mL) was added 1-methyl-4-(4,4,5,5-tetramethyl-1,3,2-dioxaborolan-2-yl)-1H-pyrazole (21.6 g, 121.2 mmol), Na₂CO₃ (25.7 g, 242.4 mmol) and Pd(dppf)Cl₂ (3 g) under N₂. The mixture was heated to reflux and stirred under N₂ for 2 h. The mixture was cooled to at room temperature, filtered. The filtrate was poured into cold water, extracted with DCM/MeOH=10/1 for 10 times, the organic phase was washed with brine, dried over Na₂SO₄, concentrated in vacuum to give crude solid. The crude solid was purified by silica gel chromatography to give S2-3 (13 g, 81.2%) as a yellow solid.

¹H NMR (CDCl₃, 400 MHZ): δ (ppm) 8.85 (d, J=0.8 Hz, 1H), 8.06 (s, 1H), 7.82 (s, 1H), 7.72-7.76 (m, 2H), 7.39 (dd, J=1.2, 9.2 Hz, 1H), 4.01 (s, 3H).

### 3. Synthesis of Compound S2

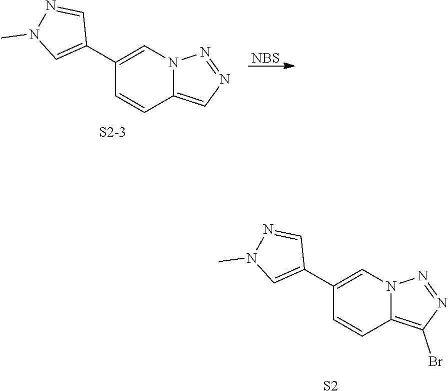

To a mixture of S2-3 (13 g, 65.3 mmol) in acetonitrile (260 mL) was added NBS (23.2 g, 130.6 mmol) in portions. The mixture was stirred at rt for 1 h, poured into water, extracted with DCM/MeOH=10/1 for 10 times, the organic phase was washed with brine, dried over Na₂SO₄, concentrated in vacuum to give crude solid. The crude solid was purified by silica gel chromatography to give S2 (2.5 g, 13.8%) as light-yellow solid.

¹H NMR (DMSO-d₆, 400 MHZ): δ (ppm) 9.44 (s, 1H), 8.40 (s, 1H), 8.12 (s, 1H), 7.78-7.86 (m, 2H), 3.90 (s, 3H).

### Synthesis of Compound S3

### 1. Synthesis of Intermediate S3-2

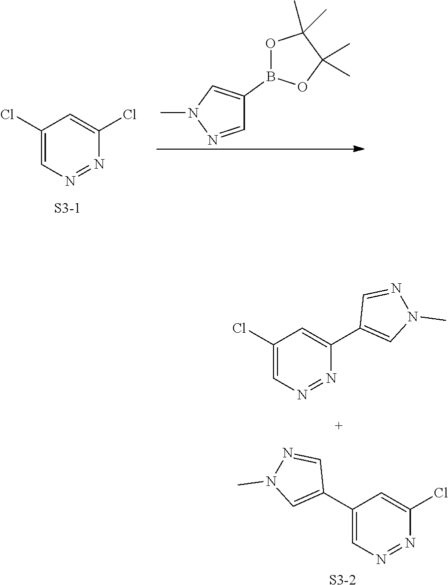

To a mixture of S3-1 (25 g, 16.8 mmol) in dioxane/water (5/1, 500 mL) was added 1-methyl-4-(4,4,5,5-tetramethyl-1,3,2-dioxaborolan-2-yl)-1H-pyrazole (34.9 g, 16.8 mmol), Na₂CO₃ (39.1 g, 36.9 mmol) and Pd(dppf)Cl₂ (5.0 g) under N₂. The mixture was heated to reflux and stirred under N₂ for 6 h. The mixture was cooled to room temperature and filtered. The filtrate was poured into cold water and extracted with DCM/MeOH=10/1 for 10 times, The organic phase was washed with brine, dried over Na₂SO₄, and concentrated in vacuum to give crude solid. The crude solid was purified by silica gel chromatography eluted with CH₂Cl₂/MeOH (100:1-50:1) to give S3-2 (17.5 g, crude) as a yellow solid.

### 2. Synthesis of Intermediate Compound S3-3

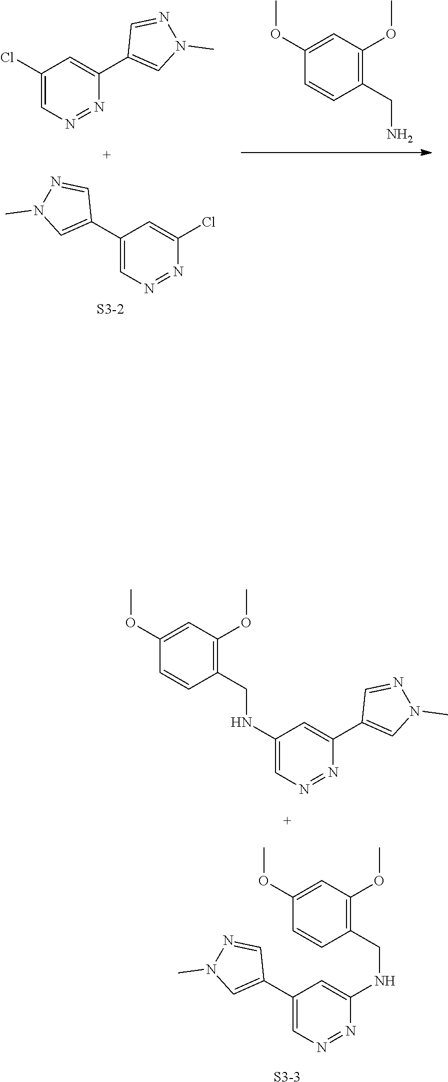

To a mixture of S3-2 (10 g, 51.4 mmol) and (2,4-dimethoxyphenyl) methanamine (19.8 g, 118.2 mmol) in DMF (200 mL) was added K₂CO₃ (21.3 g, 154.2 mmol), KI (6.8 g, 41.1 mmol) under N₂. The mixture was heated to 120° C., and stirred for 16 h. The mixture was cooled to room temperature, poured into water then extracted with DCM/MeOH=10/1 for 10 times, the organic phase was concentrated in vacuum to give crude solid. The crude solid was purified by silica gel chromatography to give crude S3-3 (12.2 g, crude) as a brown oil.

### 3. Synthesis of Intermediate Compound S3-4

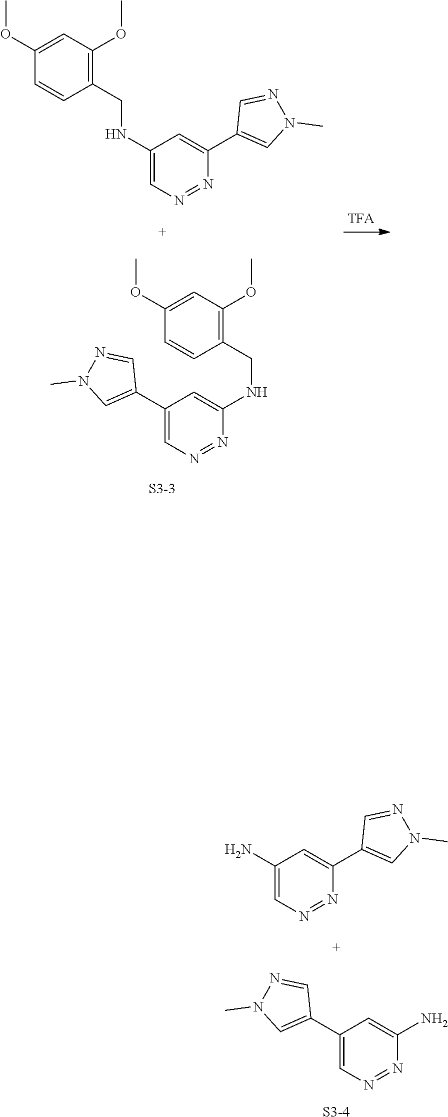

A mixture of S3-3 (10 g, 8.8 mmol) in TFA (100 mL) was stirred at 80° C. for 4 h. The mixture was concentrated in vacuum to give crude solid. The crude solid dissolved into MeOH and adjusted pH=8 with NaHCO₃. The mixture was filtered and concentrated to give a residue, which was purified by Al₂O₃ gel chromatography (DCM/MeOH=10:1˜5:1) to give S3-4 (16.6 g, crude) as a brown oil.

### 4. Synthesis of Intermediate Compound S3-5

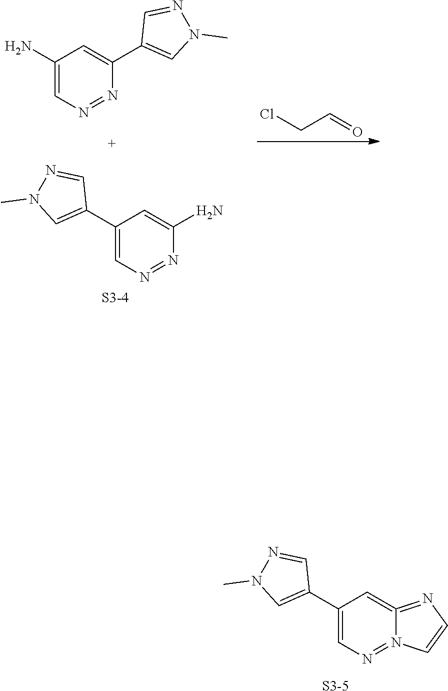

To a mixture of S3-4 (20 g, 34.2 mmol) in dioxane (400 mL) was added NaHCO₃ (17.2 g, 205.2 mmol) and 2-chloroacetaldehyde (40.2 g, 205.2 mmol, 40%, aq) under N₂. The mixture was heated to 100° C., and stirred for 6 h. The mixture was cooled to room temperature and concentrated in vacuum to give crude solid. The crude solid was purified by silica gel chromatography to give a crude, then purified by prep-HPLC to give S3-5 (0.55 g, 7.89%) as a yellow solid.

¹H NMR (DMSO-d₆, 400 MHZ): δ (ppm) 9.19 (d, J=2.0 Hz, 1H), 8.58 (s, 1H), 8.50 (d, J=1.2 Hz, 1H), 8.42 (d, J=2.0 Hz, 1H), 8.28 (s, 1H), 8.09 (d, J=1.6 Hz, 1H), 3.93 (s, 3H).

### 5. Synthesis of Compound S3

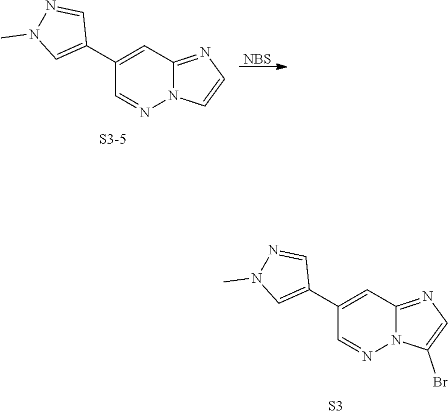

To a mixture of S3-5 (0.4 g, 2.0 mmol) in acetonitrile (15 mL) was added NBS (0.28 g, 1.6 mmol) in portions. The mixture was stirred at 10° C. for 15 min and poured into Na₂S₂O₃(aq). The mixture was extracted with DCM/MeOH=10/1 for 4 times. The organic phase was washed with Na₂S₂O₃(aq) and brine, dried over Na₂SO₄, and concentrated under vacuum to give crude solid. The crude solid was purified by prep-TLC affording S3 (0.11 g, 20.0%) as a yellow solid.

¹H NMR (CD₃OD, 400 MHZ): δ (ppm) 8.89 (d, J=2.0 Hz, 1H), 8.28 (s, 1H), 8.09-8.10 (m, 2H), 7.75 (s, 1H), 3.99 (s, 3H).

### Synthesis of Compound S4

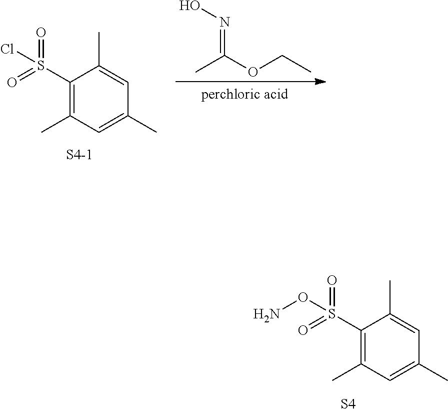

To a solution of ethyl N-hydroxyacetimidate (40.0 g, 329.2 mmol) and S4-1 (40.0 g, 182.8 mmol) in DMF (300 mL) was added triethylamine (27.7 g, 274.3 mmol) dropwise at room temperature. Then the reaction mixture is stirred at room temperature (rt) for 2 h. TLC (petroleum ether/ethyl acetate=10/1) showed the reaction was complete. The reaction mixture was then poured into ice water (900 mL). The mixture was filtered, and the filter cake was washed with H₂O (200 mL×3) and dried in vacuo to give the compound as a yellow solid. The compound was dissolved in 1,4-dioxane (26 mL), and perchloric acid (16 mL) was added dropwise at 0° C. Then the reaction mixture is stirred at 0° C. for 30 min. The reaction mixture was then poured into ice water (400 mL). The mixture was filtered and the filter cake was washed with H₂O (200 mL×3). The filter cake was dissolved in DCM (200 mL), which was used directly in the next step.

### Synthesis of Compound S5

### 1. Synthesis of Intermediate Compound S5-2

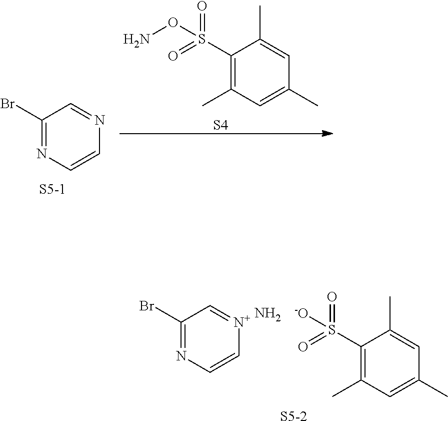

To a solution of S5-1 (22.0 g, 138.3 mmol) in DCM (154 mL) was added a solution of S4 (38.6 g, 179.8 mmol) in DCM (308 mL) at rt dropwise. Then the reaction mixture was allowed to stir overnight at rt. The mixture was concentrated in vacuo to give the crude compound. The crude product was triturated with MTBE (100 mL) and dried in vacuo to give S5-2 (34.0 g) as a black solid.

### 2. Synthesis of Intermediate Compound S5-3

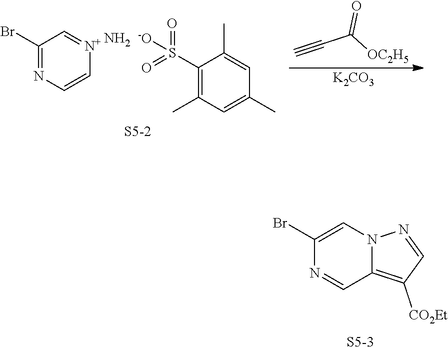

To a solution of K₂CO₃ (12.4 g, 97.4 mmol) and ethyl propiolate (27.0 g, 108.2 mmol) in DMF (270 mL) was added S5-2 (27.0 g, 72.1 mmol) in portions at room temperature, then the reaction mixture was stirred at room temperature overnight. The mixture was diluted with H₂O, the mixture was extracted with MTBE (200 mL×3). The combined organic layers were washed with brine, dried over Na₂SO₄ and filtered. The filtrate was concentrated in vacuo to give the crude compound. The crude product was purified by chromatography to give S5-3 (2.0 g, Yield: 10.3%) as a yellow solid.

¹H NMR (CDCl₃, 400 MHZ): δ (ppm) 9.41 (s, 1H), 8.66 (d, J=0.8 Hz, 1H), 8.46 (s, 1H), 4.45 (q, J=7.2 Hz, 2H), 1.45 (t, J=7.2 Hz, 3H).

### 3. Synthesis of Intermediate Compound S5-4

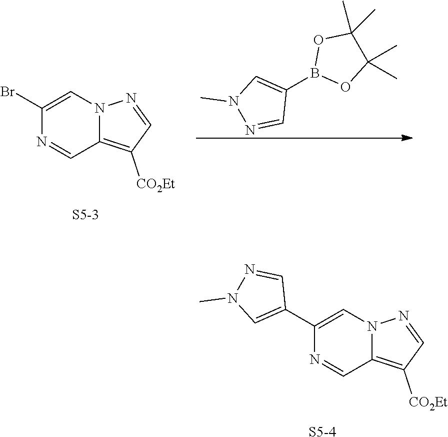

To a mixture of S5-3 (5.0 g, 18.5 mmol) in dioxane/water (80 mL/20 mL) was added 1-methyl-4-(4,4,5,5-tetramethyl-1,3,2-dioxaborolan-2-yl)-1H-pyrazole (5.7 g, 27.7 mmol), Na₂CO₃ (5.7 g, 55.4 mmol) and Pd(dppf)Cl₂ (1.0 g) under N₂. The mixture was heated to reflux and stirred under N₂ for 2.5 h. The mixture was cooled to room temperature and filtered. The filtrate was poured into cold water, extracted with DCM/MeOH=10/1 for 10 times, the organic phase was washed with brine, dried over Na₂SO₄, concentrated under vacuum to give crude solid. The crude solid was purified by silica gel chromatography to give S5-4 (3.5 g, 69.8%) as a yellow solid.

¹H NMR (CDCl₃, 400 MHZ): δ (ppm) 9.56 (d, J=1.6 Hz, 1H), 8.59 (d, J=1.2 Hz, 1H), 8.45 (s, 1H), 7.96 (d, J=7.2 Hz, 2H), 4.45 (q, J=7.2 Hz, 2H), 4.01 (s, 3H), 1.46 (t, J=7.2 Hz, 3H).

### 4. Synthesis of Intermediate Compound S5-5

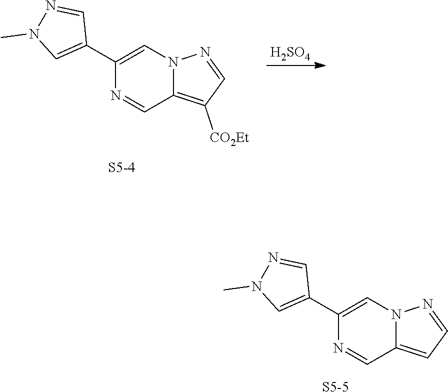

To a mixture of S5-4 (3.5 g, 12.9 mmol) in H₂SO₄ (40% aq., 35 mL) was stirred at 100° C. for overnight, LCMS showed the reaction was completed. The mixture was poured into water, then adjusted pH=8 with NaOH aq, then filtered, the filter cake was washed with water, dried to give product to give S5-5 (1.7 g, 66.1%) as yellow solid.

¹H NMR (DMSO-d₆, 400 MHZ): δ (ppm) 9.19 (d, J=1.6 Hz, 1H), 9.14 (d, J=0.8 Hz, 1H), 8.26 (s, 1H), 8.12 (d, J=2.4 Hz, 1H), 8.05 (s, 1H), 6.956 (dd, J=0.8, 2.4 Hz, 1H), 3.89 (s, 3H).

### 5. Synthesis of Compound S5

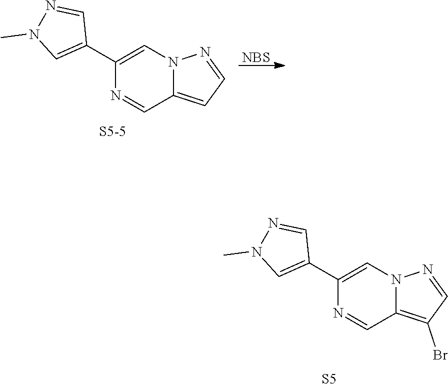

To a mixture of S5-5 (1.0 g, 5.0 mmol) in DMF (30 mL) was added NBS (1.76 g, 5.0 mmol) in portions. The mixture was stirred at rt for 1 h, poured into water, extracted with EtOAc for 3 times, the organic phase was washed with water and brine, dried over Na₂SO₄, concentrated in vacuum. The crude solid was purified by silica gel chromatography (eluted with DCM/MeOH from 1:0 to 100:1) to give S5 (600 mg, 42.8%) as a yellow solid.

¹H NMR (DMSO-d₆, 400 MHZ): δ (ppm) 9.19 (d, J=1.2 Hz, 1H), 9.09 (d, J=1.2 Hz, 1H), 8.28 (s, 2H), 8.07 (s, 1H), 3.90 (s, 3H).

### Synthesis of Compound S6

### 1. Synthesis of Intermediate Compound S6-2

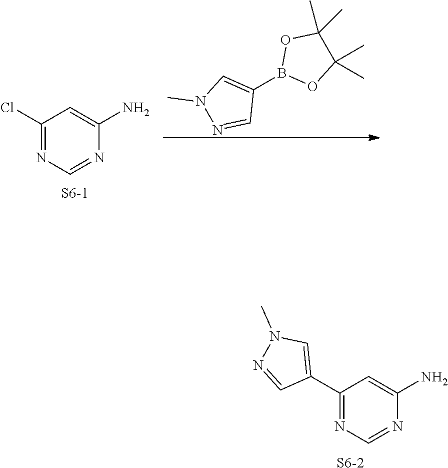

To a mixture of S6-1 (13.4 g, 103.5 mmol) in dioxane/water (260 mL/50 mL) was added 1-methyl-4-(4,4,5,5-tetramethyl-1,3,2-dioxaborolan-2-yl)-1H-pyrazole (32.3 g, 155.2 mmol), Na₂CO₃ (24 g, 227.6 mmol) and Pd(dppf)Cl₂ (3.3 g) under N₂. The mixture was heated to reflux and stirred under N₂ for 6 h. The mixture was cooled and poured into cold water, extracted with DCM/MeOH=10/1 for 10 times, the organic phase was washed with brine, dried over Na₂SO₄, concentrated in vacuum to give crude solid. The crude solid was purified by silica gel chromatography to give S6-2 (8.7 g, 48.2%) as yellow solid.

¹H NMR (DMSO-d₆, 400 MHz): δ (ppm) 8.29 (s, 1H), 8.22 (s, 1H), 7.88 (s, 1H), 6.74 (s, 2H), 6.54 (s, 1H), 3.87 (s, 3H).

### 2. Synthesis of Intermediate Compound S6-3

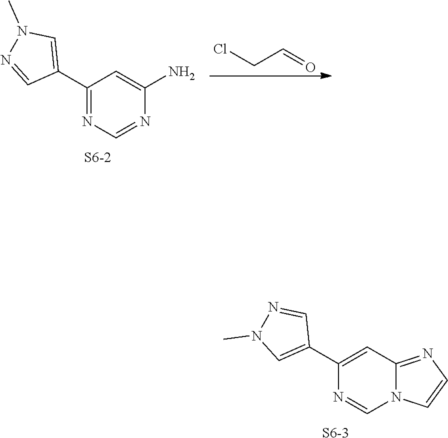

To a mixture of S6-2 (8.7 g, 50.2 mmol) in dioxane (180 mL) was added NaHCO₃ (25.3 g, 301.3 mmol), 2-chloroacetaldehyde (39.5 g, 201.0 mmol, 40%, aq) under N₂. The mixture was heated to reflux and stirred for 16 h, another batch of 2-chloroacetaldehyde (20 g, 100.5 mmol, 40%, aq) was added again. The mixture was heated to reflux and stirred for 5 h, then cooled to room temperature, concentrated in vacuum to give crude solid. The crude solid was purified by silica gel chromatography to give S6-3 (4.3 g, 43%) as a yellow solid.

¹H NMR (DMSO-d₆, 400 MHZ): δ (ppm) 9.36 (d, J=1.6 Hz, 1H), 8.29 (s, 1H), 8.06 (s, 1H), 7.96 (s, 1H), 7.81 (s, 1H), 7.59 (d, J=1.6 Hz, 1H), 3.89 (s, 3H).

### 3. Synthesis of Compound S6

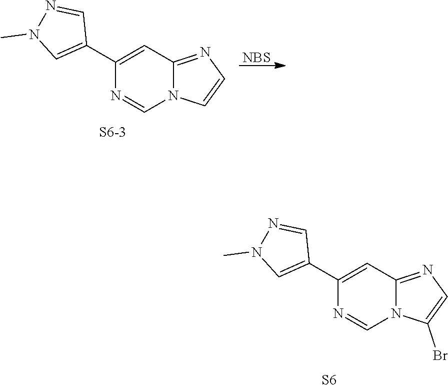

To a mixture of S6-3 (4.3 g, 21.6 mmol) in acetonitrile (120 mL) was added NBS (3.5 g, 19.4 mmol) in portions. The mixture was stirred at rt for 15 min, poured into Na₂S₂O₃(aq), extracted with DCM/MeOH=10/1 for 10 times, the organic phase was washed with Na₂S₂O₃(aq), then brine, dried over Na₂SO₄, concentrated in vacuum to give crude solid. The crude solid was purified by silica gel chromatography to give S6 (0.9 g, 15.1%) as a yellow solid.

¹H NMR (DMSO-d₆, 400 MHZ): δ (ppm) 9.17 (s, 1H), 8.33 (s, 1H), 8.10 (s, 1H), 7.87 (s, 1H), 7.72 (s, 1H), 3.90 (s, 3H).

### Synthesis of Compound S7

### 1. Synthesis of Intermediate Compound S7-2

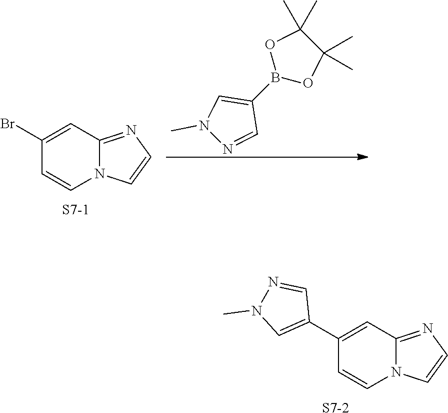

To a mixture of S7-1 (4.5 g, 22.8 mmol) in dioxane/water (100 mL/20 mL) was added 1-methyl-4-(4,4,5,5-tetramethyl-1,3,2-dioxaborolan-2-yl)-1H-pyrazole (7.1 g, 34.3 mmol), Na₂CO₃ (5.3 g, 50.3 mmol) and Pd(dppf)Cl₂ (0.9 g) under N₂. The mixture was heated to reflux and stirred under N₂ for 6 h. The mixture was cooled to at room temperature, filtered. The filtrate was poured into cold water, extracted with DCM/MeOH=10/1 for 10 times, the organic phase was washed with brine, dried over Na₂SO₄, concentrated in vacuum to give crude solid. The crude solid was purified by silica gel chromatography to give S7-2 (3.4 g, 74.7%) as a yellow solid.

¹H NMR (DMSO-d₆, 400 MHZ): δ (ppm) 8.50 (d, J=7.2 Hz, 1H), 8.29 (s, 1H), 8.03 (s, 1H), 7.86 (s, 1H), 7.76 (s, 1H), 7.52 (s, 1H), 7.14-7.16 (m, 1H), 3.88 (s, 3H).

### 2. Synthesis of Compound S7

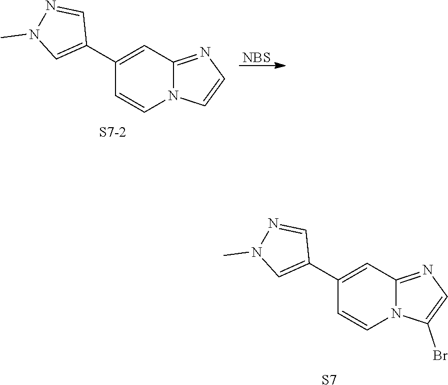

To a mixture of S7-2 (4.1 g, 20.7 mmol) in acetonitrile (120 mL) was added NBS (3.7 g, 20.7 mmol) in portions. The mixture was stirred at rt for 2 h, poured into water, extracted with DCM/MeOH=10/1 for 10 times, the organic phase was washed with brine, dried over Na₂SO₄, concentrated in vacuum to give crude solid. The crude solid was purified by silica gel chromatography to give S7 (2.2 g, 38.8%) as a yellow solid.

¹H NMR (DMSO-d₆, 400 MHZ): δ (ppm) 8.36 (s, 1H), 8.29 (d, J=7.2 Hz, 1H), 8.08 (s, 1H), 7.85 (s, 1H), 7.67 (s, 1H), 7.32-7.35 (m, 1H), 3.88 (s, 3H).

### Synthesis of Compound S8

### 1. Synthesis of Intermediate Compound S8-2

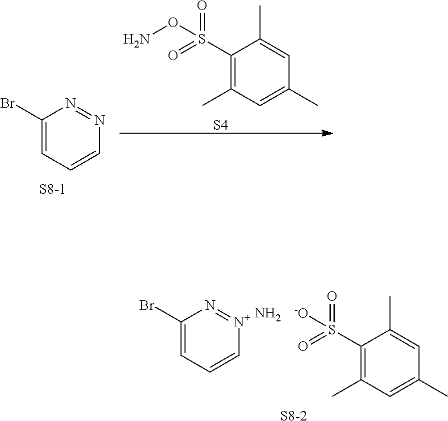

To a solution of S8-1 (20.0 g, 125.7 mmol) in DCM (300 mL) was added the solution of S4 (35.1 g, 125.7 mmol) in DCM (300 mL) at rt dropwise. Then the reaction mixture was allowed to stir overnight at rt. The mixture was concentrated in vacuo to give the crude compound. The crude product was triturated with MTBE (80 mL) and filtered to give the S8-2 (24.0 g) as black solid.

### 2. Synthesis of Intermediate Compound S8-3

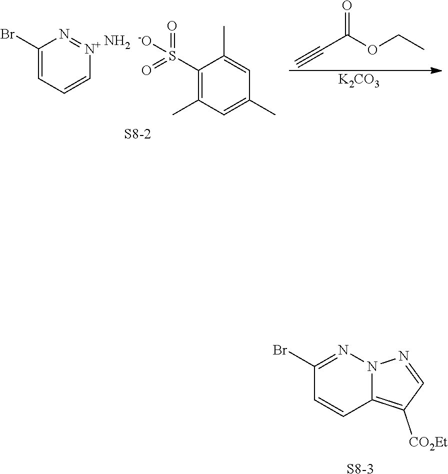

To a solution of K₂CO₃ (12.4 g, 89.8 mmol) and ethyl propiolate (7.5 g, 77.0 mmol) in DMF (230 mL) was added S8-2 (24.0 g, 64.1 mmol) in portions at room temperature. Then the reaction mixture was stirred at room temperature overnight. The mixture was diluted with H₂O, the mixture was extracted with MTBE (200 mL×3). The combined organic layers were washed with brine, dried over Na₂SO₄ and filtered. The filtrate was concentrated in vacuo to give the crude compound. The crude product was purified by chromatography to give S8-3 (2.2 g, 13.0%) as a yellow solid.

¹H NMR (CDCl₃, 400 MHZ): δ (ppm) 8.40-8.44 (m, 2H), 7.40 (d, J=9.6 Hz, 1H), 4.43 (q, J=7.2 Hz, 2H), 1.44 (t, J=7.2 Hz, 3H).

### 3. Synthesis of Intermediate Compound S8-4

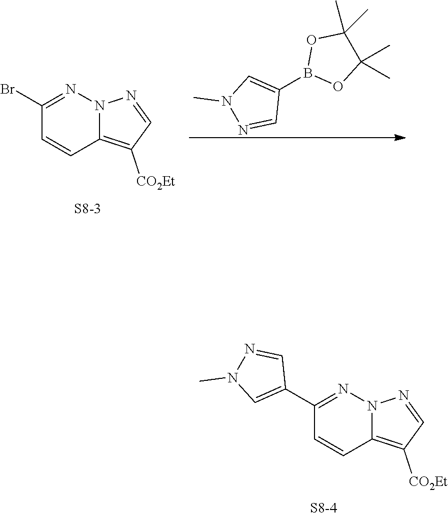

To a mixture of S8-3 (9.0 g, 33.3 mmol) in dioxane/water (144 mL/36 mL) was added 1-methyl-4-(4,4,5,5-tetramethyl-1,3,2-dioxaborolan-2-yl)-1H-pyrazole (10.4 g, 49.9 mmol), Na₂CO₃ (10.6 g, 99.9 mmol) and Pd(dppf)Cl₂ (1.0 g) under N₂. The mixture was heated to reflux and stirred under N₂ for 2 h. The mixture was cooled to room temperature, filtered. The filtrate was poured into cold water, extracted with DCM/MeOH=10/1 for 10 times, the organic phase was washed with brine, dried over Na₂SO₄, concentrated in vacuum to give crude solid. The crude solid was purified by silica gel chromatography to give S8-4 (9 g, 99.5%) as a yellow solid.

¹H NMR (CDCl₃, 400 MHZ): δ (ppm) 8.48 (d, J=9.2 Hz, 1H), 8.42 (s, 1H), 8.06 (d, J=4.4 Hz, 1H), 7.45 (d, J=9.2 Hz, 1H), 7.86 (s, 1H), 4.43 (q, J=7.2 Hz, 2H), 4.03 (s, 3H), 1.45 (t, J=7.2 Hz, 3H).

### 4. Synthesis of Intermediate Compound S8-5

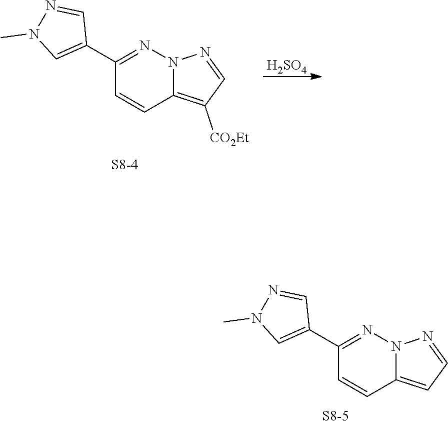

To a mixture of S8-4 (4.5 g, 16.6 mmol) in H2SO₄ (40% aq., 45 mL) was stirred at 100° C. for overnight, LCMS showed the reaction was completed. The mixture was poured into water, extracted with DCM/MeOH=10/1 for 10 times, the organic phase was washed with brine, dried over Na₂SO₄, concentrated in vacuum to give crude solid. The crude solid was washed with MTBE to give S8-5 (2.5 g, 75.7%) as a yellow solid.

¹H NMR (CDCl₃, 400 MHZ): δ (ppm) 8.04 (d, J=7.2 Hz, 2H), 7.98 (d, J=2.4 Hz, 1H), 7.93 (d, J=9.6 Hz, 1H), 7.21 (d, J=9.2 Hz, 1H), 6.61 (d, J=2.4 Hz, 1H), 4.00 (s, 3H).

### 5. Synthesis of Compound S8

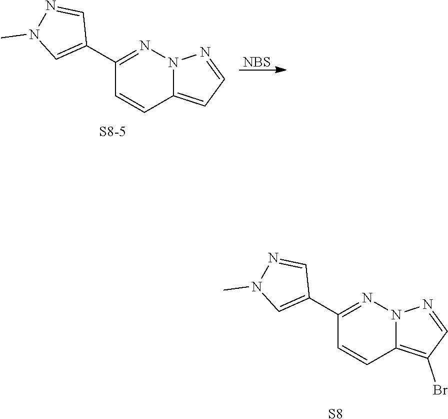

To a mixture of S8-5 (2.5 g, 12.5 mmol) in acetonitrile (50 mL) was added NBS (2.2 g, 12.5 mmol) in portions. The mixture was stirred at rt for 1 hr, poured into water, extracted with DCM/MeOH=10/1 for 10 times, the organic phase was washed with brine, dried over Na₂SO₄, concentrated in vacuum to give S8 (2.5 g, 71.4%) as a yellow solid.

¹H NMR (DMSO-d₆, 400 MHZ): δ (ppm) 8.51 (s, 1H), 8.14-8.19 (m, 3H), 7.65 (d, J=9.6 Hz, 1H), 3.93 (s, 3H).

### Synthesis of Compound S9

### 1. Synthesis of Intermediate Compound S9-2

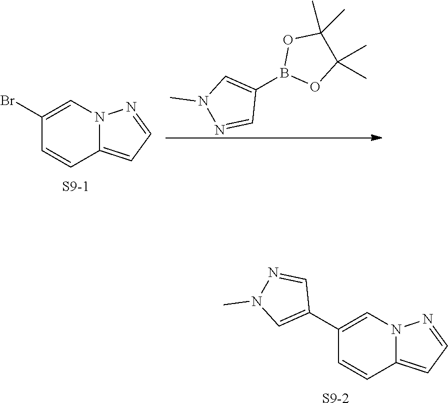

To a mixture of S9-1 (4.5 g, 22.8 mmol) in dioxane/water (90 mL/18 mL) was added 1-methyl-4-(4,4,5,5-tetramethyl-1,3,2-dioxaborolan-2-yl)-1H-pyrazole (7.1 g, 34.2 mmol), Na₂CO₃ (7.2 g, 68.8 mmol) and Pd(dppf)Cl₂ (0.45 g) under N₂. The mixture was heated to reflux and stirred under N₂ for 3 h. The mixture was cooled to at room temperature and filtered. The filtrate was poured into cold water, extracted with DCM/MeOH=10/1 for 10 times, the organic phase was washed with brine, dried over Na₂SO₄, concentrated under vacuum to give crude solid. The crude solid was purified by silica gel chromatography to give S9-2 (3.5 g, 77.7%) as a yellow solid.

¹H NMR (CDCl₃, 400 MHz): δ (ppm) 8.61 (s, 1H), 7.95 (d, J=1.6 Hz, 1H), 7.77 (s, 1H), 7.63 (s, 1H), 7.57 (d, J=9.2 Hz, 1H), 7.23-7.26 (m, 1H), 6.52 (d, J=1.6 Hz, 1H), 3.99 (s, 3H).

### 2. Synthesis of Compound S9

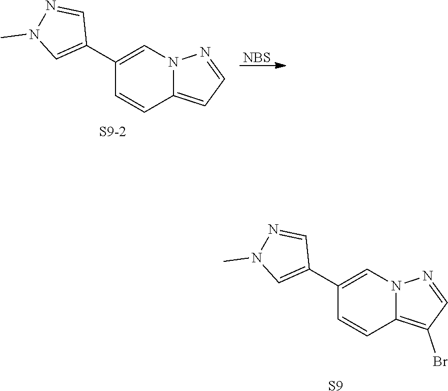

To a mixture of S9-2 (4.0 g, 20.2 mmol) in acetonitrile (80 mL) was added NBS (4.3 g, 24.2 mmol) in portions. The mixture was stirred at rt for 1h, poured into water, extracted with DCM/MeOH=10/1 for 10 times. The organic phase was washed with brine, dried over Na₂SO₄, concentrated in vacuum to give crude solid. The crude solid was purified by silica gel chromatography to give S9 (2.5 g, 44.6%) as a yellow solid.

¹H NMR (CDCl₃, 400 MHZ): δ (ppm) 8.55 (s, 1H), 7.92 (s, 1H), 7.77 (s, 1H), 7.65 (s, 1H), 7.53 (d, J=8.8 Hz, 1H), 7.53 (dd, J=0.8, 9.2 Hz, 1H), 4.00 (s, 3H).

### Synthesis of Compound S10

### 1. Synthesis of Intermediate Compound S10-2

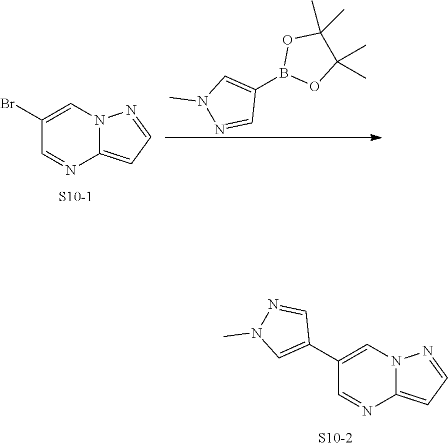

To a mixture of S10-1 (4.5 g, 22.7 mmol) in dioxane/water (90 mL/18 mL) was added 1-methyl-4-(4,4,5,5-tetramethyl-1,3,2-dioxaborolan-2-yl)-1H-pyrazole (7.0 g, 34.1 mmol), Na₂CO₃ (7.2 g, 68.1 mmol) and Pd(dppf)Cl₂ (0.45 g) under N₂. The mixture was heated to reflux and stirred under N₂ for 3 h. The mixture was cooled to at room temperature, filtered. The filtrate was poured into cold water, extracted with DCM/MeOH=10/1 for 10 times, the organic phase was washed with brine, dried over Na₂SO₄, concentrated in vacuum to give crude solid. The crude solid was purified by silica gel chromatography to give S10-2 (3.5 g, 77.7%) as a yellow solid.

¹H NMR (CDCl₃, 400 MHZ): δ (ppm) 8.75-8.76 (m, 1H), 8.64 (d, J=2 Hz, 1H), 8.12 (d, J=2.4 Hz, 1H), 7.80 (s, 1H), 7.70 (s, 1H), 6.72-6.73 (m, 1H), 4.02 (s, 3H).

### 2. Synthesis of Compound S10

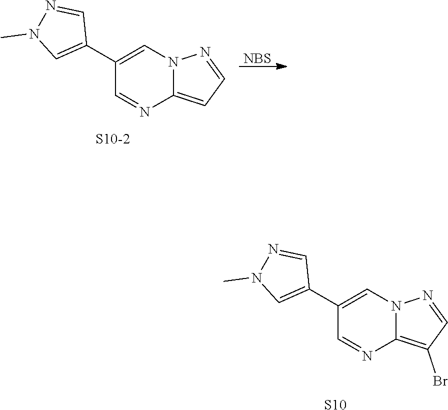

To a mixture of S10-2 (7.0 g, 35.1 mmol) in acetonitrile (140 mL) was added NBS (7.5 g, 42.1 mmol) in portions. The mixture was stirred at rt for 1 h, poured into water, extracted with DCM/MeOH=10/1 for 10 times, the organic phase was washed with brine, dried over Na₂SO₄, concentrated in vacuum to give crude solid. The crude solid was purified by silica gel chromatography to give S10 (5 g, 51.0%) as a yellow solid.

¹H NMR (CDCl₃, 400 MHZ): δ (ppm) 8.72-8.69 (m, 2H), 8.10 (s, 1H), 7.79 (s, 1H), 7.72 (s, 1H), 4.02 (s, 3H).

### Synthesis of Compound S11

### 1. Synthesis of Intermediate Compound S11-2

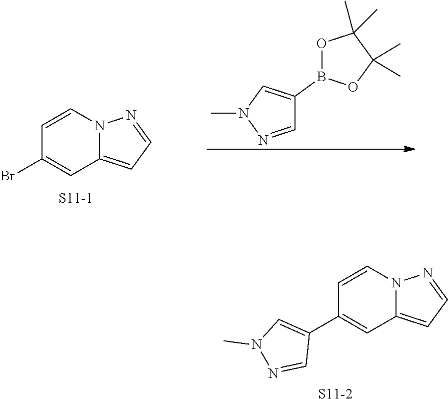

To a mixture of S11-1 (6.8 g, 34.5 mmol) in dioxane/water (136 mL/27 mL) was added 1-methyl-4-(4,4,5,5-tetramethyl-1,3,2-dioxaborolan-2-yl)-1H-pyrazole (10.8 g, 51.8 mmol), Na₂CO₃ (8.0 g, 75.9 mmol) and Pd(dppf)Cl₂ (1.4 g) under N₂. The mixture was heated to reflux and stirred under N₂ for 6 h. The mixture was cooled to at room temperature and filtered. The filtrate was poured into cold water, extracted with DCM/MeOH=10/1 for 10 times, the organic phase was washed with brine, dried over Na₂SO₄, concentrated in vacuum to give crude solid. The crude solid was purified by silica gel chromatography to give S11-2 (6.0 g, 88%) as a yellow solid.

¹H NMR (CDCl₃, 400 MHZ): δ (ppm) 8.45 (d, J=7.2 Hz, 1H), 7.94 (d, J=2.0 Hz, 1H), 7.83 (s, 1H), 7.69 (s, 1H), 7.60 (d, J=1.2 Hz, 1H), 6.86-6.88 (m, 1H), 6.48-6.49 (m, 1H), 3.99 (s, 3H).

### 2. Synthesis of Compound S11

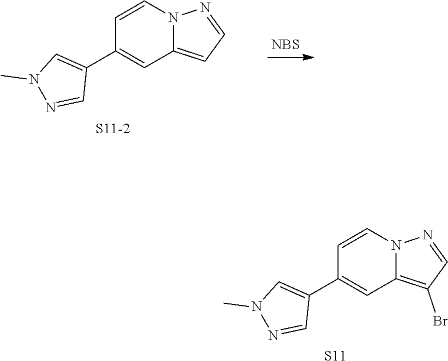

To a mixture of S11-2 (6.0 g, 30.3 mmol) in acetonitrile (120 mL) was added NBS (5.4 g, 30.3 mmol) in portions. The mixture was stirred at rt for 2 h, poured into water, extracted with DCM/MeOH=10/1 for 10 times, the organic phase was washed with brine, dried over Na₂SO₄, concentrated in vacuum to give crude solid. The crude solid was purified by silica gel chromatography to give S11 (4.2 g, 50.8%) as a white solid.

¹H NMR (CDCl₃, 400 MHZ): δ (ppm) 8.39-8.41 (m, 1H), 7.91 (s, 1H), 7.87 (s, 1H), 7.75 (s, 1H), 7.53 (d, J=1.2 Hz, 1H), 6.91-6.93 (m, 1H), 4.00 (s, 3H).

### Synthesis of Compound S12

### 1. Synthesis of Intermediate Compound S12-2

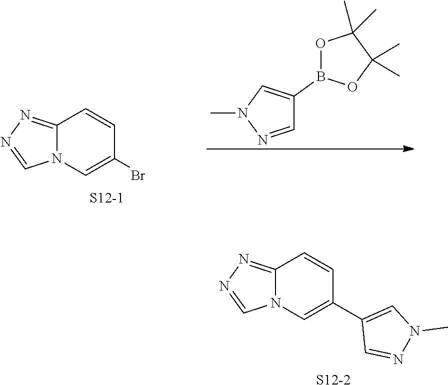

To a mixture of S12-1 (7.7 g, 38.9 mmol) in dioxane/water (160 mL/30 mL) was added 1-methyl-4-(4,4,5,5-tetramethyl-1,3,2-dioxaborolan-2-yl)-1H-pyrazole (12.1 g, 58.3 mmol), K₂CO₃ (11.8 g, 85.5 mmol) and Pd(PPh₃)₄ (1.5 g) under N₂. The mixture was heated to reflux and stirred under N₂ for 6 h. The mixture was cooled to at room temperature, then concentrated in vacuum to give crude solid. The crude solid was purified by silica gel chromatography to give S12-2 (7.6 g, 98.7%) as a yellow solid.

¹H NMR (DMSO-d₆, 400 MHZ): δ (ppm) 9.19 (s, 1H), 8.80 (s, 1H), 8.22 (s, 1H), 7.92 (s, 1H), 7.80 (d, J=9.6 Hz, 1H), 7.62-7.65 (m, 1H), 3.89 (s, 3H).

### 2. Synthesis of Compound S12

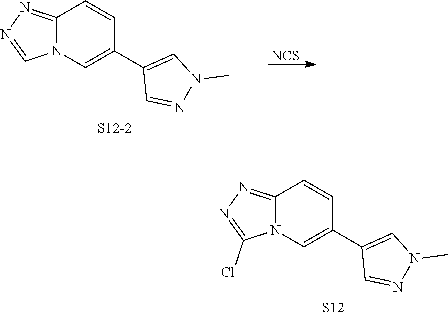 [SMILES: Cn1cc(-c2ccc3nncn3c2)cn1 ; "NCS" ; "S12-2" ; Cn1cc(-c2ccc3nnc(Cl)n3c2)cn1 ; "S12"]

To a mixture of S12-2 (7.6 g, 38.2 mmol) in DMF (150 mL) was added NCS (5.1 g, 38.2 mmol) in portions. The mixture was stirred at 25-30° C. for 16 h. The reaction was quenched by the addition of water, concentrated in vacuum to give crude solid. The crude solid was purified by silica gel chromatography to give S12 (4.1 g, 46%) as a chartreuse solid.

¹H NMR (DMSO-d₆, 400 MHZ): δ (ppm) 9.45 (s, 1H), 8.36 (s, 1H), 8.01 (s, 1H), 7.87 (d, J=9.6 Hz, 1H), 7.71 (d, J=9.6 Hz, 1H), 3.93 (s, 3H).

### Synthesis of Compound S13

### 1. Synthesis of Intermediate Compound S13-2

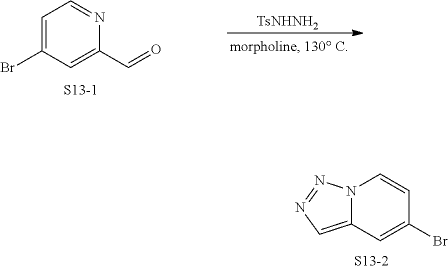 [SMILES: "TsNHNH2" ; O=Cc1cc(Br)ccn1 ; "S13-1" ; Brc1ccn2nncc2c1 ; "S13-2"]

To a mixture of S13-1 (14.5 g, 78 mmol) in MeOH (220 mL) was added TsNHNH₂ (15.2 g, 81.9 mmol) at room temperature. The mixture was stirred at room temperature for 2 h. The mixture was concentrated in vacuum to give the solid. The solid was dissolved in morpholine (140 mL). The mixture was stirred at 130° C. for 3 h. Then the mixture was poured into cold water, extracted with EtOAc. The organic phase was washed with water, brine, dried over Na₂SO₄, concentrated in vacuum to give crude solid. The crude solid was triturated with petroleum ether and filtered to give S13-2 (12.0 g, 77.9%) as an off-white solid.

¹H NMR (DMSO-d₆, 400 MHZ): δ (ppm) 9.06 (d, J=7.2 Hz, 1H), 8.33 (d, J=1.2 Hz, 1H), 8.17 (d, J=0.8 Hz, 1H), 7.31 (dd, J=2.0, 7.6 Hz, 1H).

### 2. Synthesis of Intermediate Compound S13-3

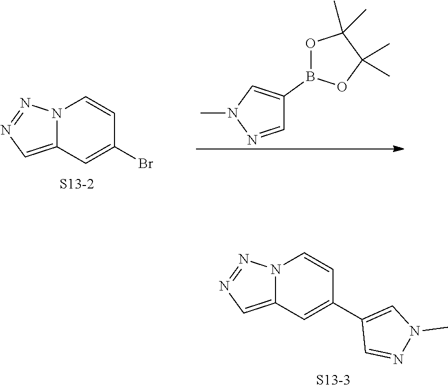

To a mixture of S13-2 (12 g, 60.6 mmol) in dioxane/water (240 mL/48 mL) was added 1-methyl-4-(4,4,5,5-tetramethyl-1,3,2-dioxaborolan-2-yl)-1H-pyrazole (18.9 g, 90.9 mmol), Na₂CO₃ (19.3 g, 181.8 mmol) and Pd(dppf)Cl₂ (1.2 g) under N₂. The mixture was stirred at reflux under N₂ for 4 h. The mixture was cooled to room temperature, poured into cold water, extracted with DCM/MeOH=10/1. The organic phase was washed with water, brine, dried over Na₂SO₄, concentrated in vacuum to give crude solid. The crude solid was triturated with MTBE and filtered to give S13-3 (9.8 g, 81.0%) as a brown solid.

¹H NMR (DMSO-d₆, 400 MHz): δ (ppm) 9.06 (d, J=7.2 Hz, 1H), 8.37 (s, 1H), 8.08-8.10 (m, 3H), 7.41 (dd, J=1.6, 7.2 Hz, 1H), 3.89 (s, 3H).

### 3. Synthesis of Compound S13

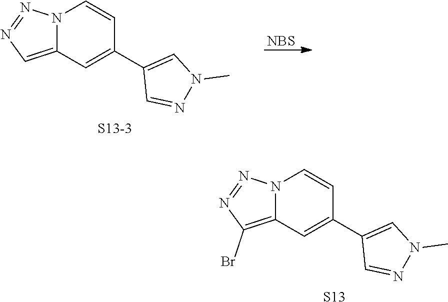

To a mixture of S13-3 (9.8 g, 49.2 mmol) in acetonitrile (196 mL) was added NBS (10.5 g, 59.0 mmol) at room temperature. The mixture was stirred at 90° C. for 2 h. The mixture was concentrated in vacuum. To the residue was added water, and then extracted with DCM/MeOH=10/1. The organic phase was washed with water, brine, dried over Na₂SO₄, concentrated in vacuum to give crude oil. The crude oil was purified by silica gel chromatography to give crude product, which was triturated with MTBE and filtered to give S13 (4.38 g, 32%) as an off-white solid.

¹H NMR (DMSO-d₆, 400 MHZ): δ (ppm) 9.12 (dd, J=0.8, 7.2 Hz, 1H), 8.50 (s, 1H), 8.20 (s, 1H), 7.95 (s, 1H), 7.54 (dd, J=1.6, 7.2 Hz, 1H), 3.90 (s, 3H).

### Synthesis of Compound S14

### 1. Synthesis of Intermediate Compound S14-2

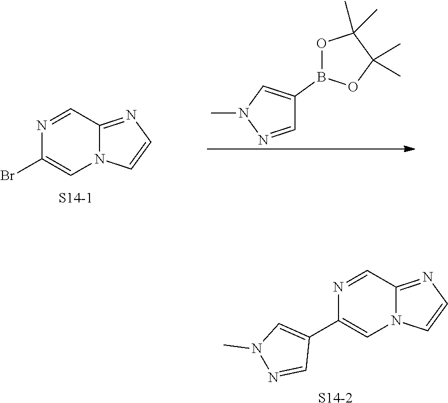

To a mixture of S14-1 (6.5 g, 32.8 mmol) in dioxane/water (130 mL/26 mL) was added 1-methyl-4-(4,4,5,5-tetramethyl-1,3,2-dioxaborolan-2-yl)-1H-pyrazole (10.2 g, 49.2 mmol), Na₂CO₃ (10.4 g, 98.4 mmol) and Pd(dppf)Cl₂ (0.65 g) under N₂. The mixture was heated to reflux and stirred under N₂ for 6 h. The mixture was cooled to at room temperature, poured into cold water, extracted with DCM/MeOH=10/1, the organic phase was washed with brine, dried over Na₂SO₄, concentrated in vacuum to give crude solid. The crude solid was triturated with MTBE and filtered to give S14-2 (6.2 g, 95.3%) as a brown solid.

¹H NMR (DMSO-d₆, 400 MHZ): δ (ppm) 9.06 (s, 1H), 8.90 (d, J=1.2 Hz, 1H), 8.19 (s, 1H), 8.07 (s, 1H), 7.93 (s, 1H), 7.80 (s, 1H), 3.90 (s, 3H).

### 2. Synthesis of Compound S14

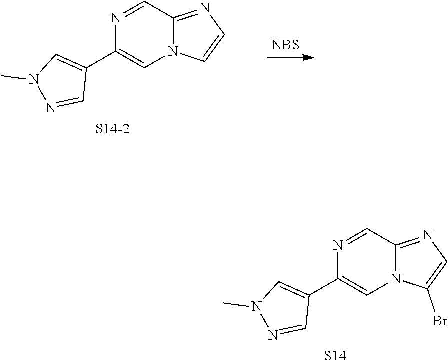

To a mixture of S14-2 (6.2 g, 31.1 mmol) in DMF (120 mL) was added NBS (6.6 g, 37.3 mmol) at room temperature. The mixture was stirred at 90° C. for 2 h. The reaction was cooled to room temperature, poured into cold water, and extracted with EtOAc. The organic phase was washed with water, brine, dried over Na₂SO₄, and concentrated under vacuum to give a crude solid. The crude solid was purified by silica gel chromatography to give S14 (3.7 g, 42.9%) as a brown solid.

¹H NMR (DMSO-d₆, 400 MHZ): δ (ppm) 9.08 (d, J=1.2 Hz, 1H), 8.60 (d, J=1.2 Hz, 1H), 8.36 (s, 1H), 8.10 (d, J=0.8 Hz, 1H), 7.95 (s, 1H), 3.90 (s, 3H).

### Synthesis of Compound S15

### 1. Synthesis of Intermediate Compound S15-2

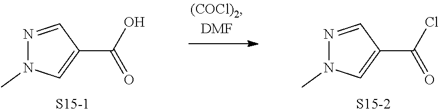

To a solution of S15-1 (50 g, 397 mmol) and oxalyl chloride (75.5 g, 594.7 mmol) in DCM (1000 mL) was added DMF (7.5 mL, cat) dropwise under N₂ at 0° C. The reaction mixture was stirred at 25° C. for 3 h. The mixture was concentrated in vacuum to give the crude product (55 g), which was used in the next step directly.

### 2. Synthesis of Intermediate Compound S15-3

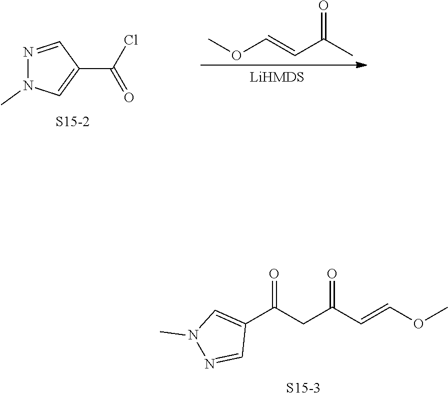

The solution of LiHMDS (958.9 mL, 958.9 mmol) in THF (950 mL) is cooled to −78° C. in a dry ice-acetone bath. The flask is three-way stopcock attached to a balloon filled with nitrogen. To the stirred solution is added 4-methoxy-3-buten-2-one (60 g, 59.9 mmol) in THF (250 mL). The mixture is stirred for 2 h at −78° C., then S15-2 (55 g, crude) in THF (300 mL) was added dropwise at −78° C., while it is allowed to warm to room temperature for 2 h. The reaction mixture was poured into aqueous NH₄Cl (1000 mL), then extracted with EtOAc (1000 mL×2) The organic layer was washed with brine, dried over Na₂SO₄, concentrated under vacuum to give the crude product, which was purified by chromatography on silica gel (PE/EtOAc from 8/1 to 5/1) to give S15-3 (17 g, 20.6% for two steps) as a yellow solid.

¹H NMR (DMSO-d₆, 400 MHZ): δ (ppm) 8.31 (s, 1H), 7.94 (s, 1H), 7.68 (d, J=12.4 Hz, 1H), 6.12 (s, 1H), 5.53 (d, J=12.8 Hz, 1H), 3.88 (s, 3H), 3.74 (s, 3H).

### 3. Synthesis of Intermediate Compound S15-4

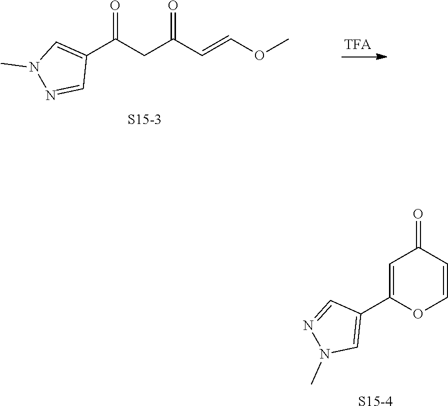

A round-bottom flask containing a mixture of S15-3 (17 g, 81.7 mmol) and TFA (10 mL) in toluene (200 mL) was placed in oil bath heated to 78° C. for 3 h. The mixture was concentrated in vacuum to give the crude product, and the residue was partitioned between DCM/MeOH=10/1 and water. The organic layer was washed with brine, dried over Na₂SO₄, then concentrated in vacuum to give S15-4 (12 g, 83.4%) as a yellow solid.

¹H NMR (CDCl₃, 400 MHZ): δ (ppm) 7.82 (s, 1H), 7.77 (s, 1H), 7.74 (d, J=5.6 Hz, 1H), 6.50 (d, J=2.4 Hz, 1H), 6.32-6.34 (m, 1H), 3.98 (s, 3H).

### 4. Synthesis of Intermediate Compound S15-5

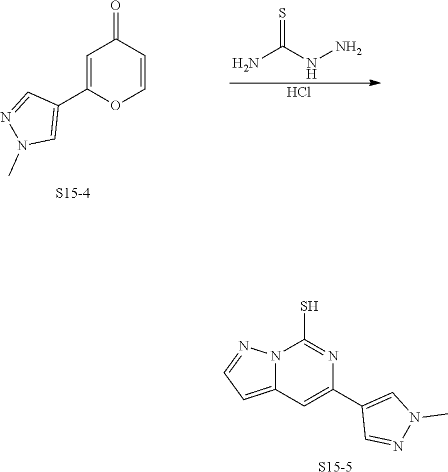 [SMILES: "S15-4" ; Cn1cc(-c2cc(=O)cco2)cn1 ; NNC(N)=S ; Cl ; "S15-5" ; Cn1cc(-c2cc3ccnn3c(S)n2)cn1]

To a solution of S15-4 (12.0 g, 68.1 mmol), hydrazinecarbothioamide (18.6 g, 204.3 mmol) and in MeCN (600 mL) was stirred under nitrogen under argon at 0° C. was added a solution of HCl (28.4 mL, 340.5 mmol). The reaction mixture was stirred at rt for 20 min, then 78° C. for 16 h. The mixture was concentrated in vacuum to give the crude product S15-5 (25 g, crude) as a yellow oil, which was used in the next step directly.

### 5. Synthesis of Intermediate Compound S15-6

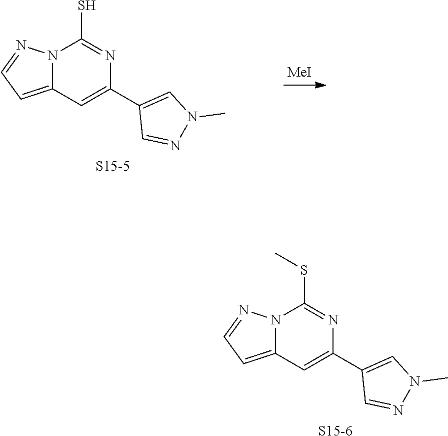 [SMILES: "MeI" ; "S15-5" ; Cn1cc(-c2cc3ccnn3c(S)n2)cn1 ; CSc1nc(-c2cnn(C)c2)cc2ccnn12 ; "S15-6"]

To a solution of S15-5 (25 g, crude) and Sodium hydroxide (13.6 g, 0.34 mol) in (MeOH/H₂O=2:1, 600 mL) stirred under nitrogen at 0° C. was added methyl iodide (48.3 g, 0.34 mmol) dropwise. The reaction mixture was stirred at 25° C. for 2 h. The mixture was adjusted to pH=6 with 2N HCl, EtOAc was added to the residue, and the organic layer was separated. The organic layer was dried over Na₂SO₄ and concentrated in vacuum. The crude product was chromatographed on silica gel to give S15-6 (3.3 g, 19.8%) as a yellow solid.

¹H NMR (DMSO-d₆, 400 MHZ): δ (ppm) 8.33 (s, 1H), 8.13 (d, J=2.4 Hz, 1H), 8.06 (s, 1H), 7.69 (s, 1H), 6.63 (d, J=2.0 Hz, 1H), 3.91 (s, 3H), 2.74 (s, 3H).

### 6. Synthesis of Intermediate Compound S15-7

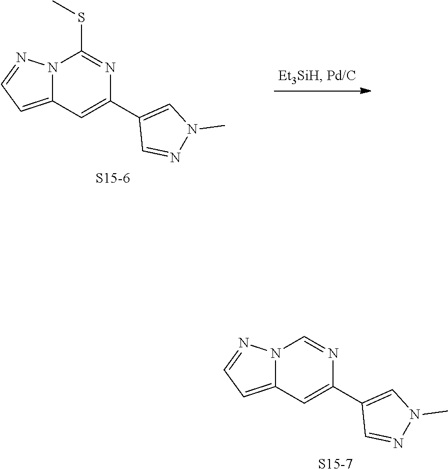 [SMILES: CSc1nc(-c2cnn(C)c2)cc2ccnn12 ; "S15-6" ; Cn1cc(-c2cc3ccnn3cn2)cn1 ; "S15-7"]

To a solution of S15-6 (3.3 g, crude) and triethylsilane (33 g) in THF (50 mL) stirred under nitrogen at 0° C. was added Pd/C (3.3 g, 10% wt) portionwise. The reaction mixture was stirred at 25° C. for 5 h. The mixture was filtered through a Celite pad, and the filtrate was concentrated in vacuum to give crude product. The crude product was chromatographed on silica gel (PE/EtOAc from 5:1 to 3:1) to give S15-7 (1.22 g, 45.5%) as a pink solid.

¹H NMR (DMSO-d₆, 400 MHZ): δ (ppm) 9.48 (s, 1H), 8.27 (s, 1H), 8.16 (d, J=2.0 Hz, 1H), 8.02 (s, 1H), 7.88 (d, J=1.2 Hz, 1H), 6.59-6.60 (m, 1H), 3.89 (s, 3H).

### 7. Synthesis of Compound S15

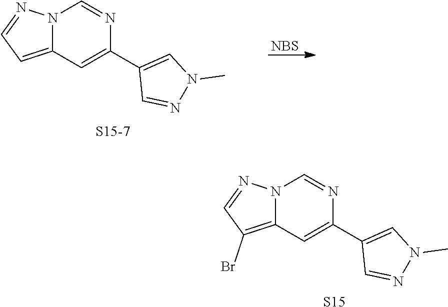

To a mixture of S15-7 (1.1 g, 5.5 mmol) in acetonitrile (33 mL) was added NBS (0.98 g, 5.5 mmol) in portions. The mixture was stirred at rt for 20 min, poured into Na₂S₂O₃(aq), extracted with DCM/MeOH=10/1 for 3 times, the organic phase was washed with Na₂S₂O₃(aq), then brine, dried over Na₂SO₄, concentrated in vacuum to give crude solid. The crude solid was purified by silica gel chromatography to give S15 (1.06 g, 67.3%) as a white solid.

¹H NMR (DMSO-d₆, 400 MHZ): § (ppm) 9.50 (s, 1H), 8.39 (s, 1H), 8.31 (s, 1H), 8.13 (s, 1H), 7.77 (d, J=1.2 Hz, 1H), 3.90 (s, 3H).

### Synthesis of Compound S16

### 1. Synthesis of Intermediate Compound S16-2

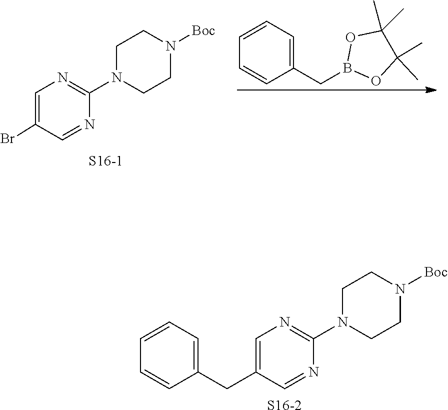

To a mixture of S16-1 (129.5 g, 0.38 mol) in dioxane/water (1950 mL/390 mL) was added 2-benzyl-4,4,5,5-tetramethyl-1,3,2-dioxaborolane (124.3 g, 0.57 mol), Na₂CO₃ (121 g, 1.14 mol) and Pd(dppf)Cl₂ (13.0 g) under N₂. The mixture was heated to reflux and stirred under N₂ for overnight. The mixture was cooled to at room temperature and filtered. The filtrate was poured into cold water and extracted with EtOAc. The organic phase was washed with brine, dried over Na₂SO₄, and concentrated under vacuum to give crude solid. The crude solid was purified by silica gel chromatography to give an off-white solid (108 g), which was triturated with petroleum ether and filtered to give S16-2 (85 g, 63%) as an off-white solid.

¹H NMR (DMSO-d₆, 400 MHZ): δ (ppm) 8.30 (s, 2H), 7.21-7.29 (m, 5H), 3.78 (s, 2H), 3.65-3.68 (m, 4H), 3.33-3.38 (m, 4H), 1.41 (s, 9H).

### 2. Synthesis of Compound S16

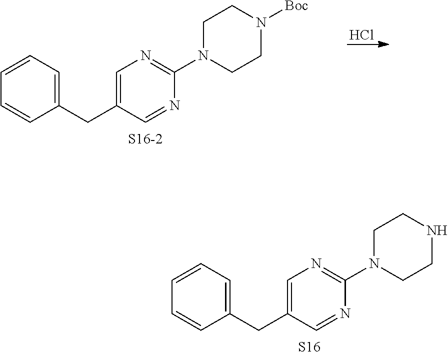 [SMILES: CC(C)(C)OC(=O)N1CCN(c2ncc(Cc3ccccc3)cn2)CC1 ; Cl ; "S16-2" ; c1ccc(Cc2cnc(N3CCNCC3)nc2)cc1 ; "S16"]

To a mixture of S16-2 (85 g, 0.24 mol) in MeOH (150 mL) was added HCl/MeOH (8 M, 850 mL) at room temperature. The mixture was stirred at room temperature for 2 h. The mixture was concentrated in vacuum to give a solid. The solid was dissolved in water (400 mL), and stirred for 10 min. The pH of the solution was adjusted to 8-9 with aq. Na₂CO₃, then extracted with DCM/MeOH (10/1), washed with water, brine, dried over Na₂SO₄, filtered, and concentrated under vacuum to give S16 (52 g, 85%) as an off-white solid.

¹H NMR (DMSO-d₆, 400 MHZ): δ (ppm) 8.32 (s, 2H), 7.19-7.29 (m, 5H), 3.79-3.83 (m, 6H), 2.95-2.98 (m, 4H).

### Synthesis of Compound S17

### 1. Synthesis of Intermediate Compound S17-2

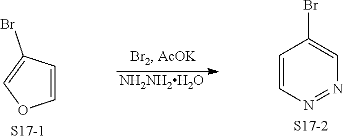

To a solution of S17-1 (30.0 g, 204.1 mmol) and potassium acetate (55.0 g, 551.0 mmol) in AcOH (80 mL) was added a solution of Br₂ (10.5 mL) in AcOH (60 mL) dropwise at rt, then the reaction mixture was stirred at rt for 1 hr. The reaction mixture was filtered, and the filtrate was concentrated in vacuo. The residue was dissolved in ethanol (300 mL) and hydrazine hydrate (30 mL) was added dropwise to the solution, which was then stirred at rt for two hours. The mixture was diluted with H₂O, then the mixture was extracted with DCM (100 mL×3). The combined organic layers were washed with brine, dried over Na₂SO₄, and filtered. The filtrate was concentrated in vacuo to give the crude compound. The crude product was purified by chromatography to give S17-2 (30 g, crude) as a black oil.

¹H NMR (CDCl₃, 400 MHZ): δ (ppm) 9.33-9.32 (m, 1H), 9.02-9.00 (m, 1H), 7.71-7.69 (m, 1H).

### 2. Synthesis of Intermediate Compound S17-3

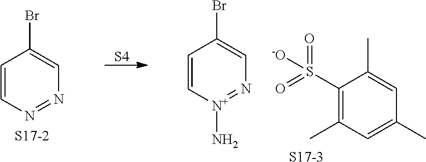

To a solution of S17-2 (30.0 g, 189.8 mmol) in DCM (200 mL) was added a solution of S4 (28.0 g, 130.2 mmol) in DCM (300 mL) at rt. Then the reaction mixture was allowed to stir overnight at rt. The mixture was concentrated in vacuo to give the crude compound, the crude compound was triturated with MTBE to give S17-3 (53.0 g, crude) as a black solid.

¹H NMR (CDCl₃, 400 MHZ): δ (ppm) 7.30 (s, 1H), 7.17 (s, 1H), 7.05 (s, 1H), 6.76 (s, 2H), 2.50 (s, 6H), 2.17 (s, 3H).

### 3. Synthesis of Intermediate Compound S17-4

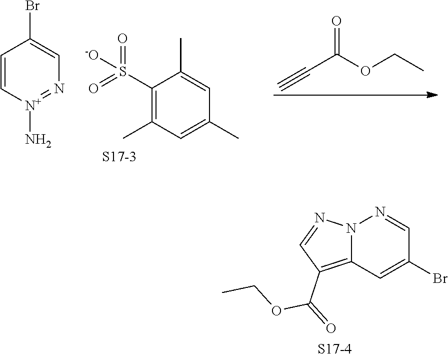

To a solution of K₂CO₃ (15.7 g, 0.142 mmol) and ethyl propiolate (11.9 g, 0.122 mol) in DMF (360 mL) was added S17-3 (40.0 g, 106.9 mmol) in portions at room temperature. Then the reaction mixture is stirred at room temperature overnight. The mixture was diluted with H₂O, the mixture was extracted with MTBE (300 mL×3). The combined organic layers were washed with water and brine, dried over Na₂SO₄, and filtered. The filtrate was concentrated in vacuo to give the crude compound. The crude product was purified by chromatography to give S17-4 (0.9 g, Yield: 3.0%) as a yellow solid.

¹H NMR (CDCl₃, 400 MHZ): δ (ppm) 8.72 (d, J=2.4 Hz, 1H), 8.47 (s, 1H), 8.45 d, J=2.4 Hz, 1H), 4.43 (q, J=7.2 Hz, 2H), 1.44 (t, J=7.2 Hz, 3H).

### 4. Synthesis of Intermediate Compound S17-5

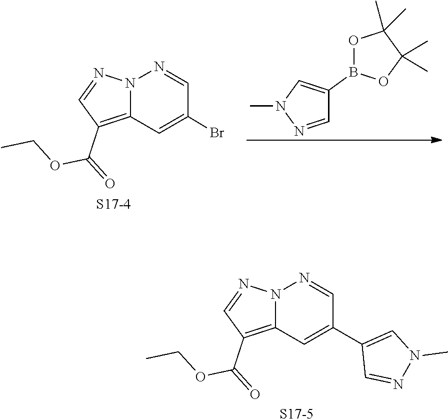

To a mixture of S17-4 (3.5 g, 12.9 mmol) in dioxane/water (56 mL/11 mL) was added 1-methyl-4-(4,4,5,5-tetramethyl-1,3,2-dioxaborolan-2-yl)-1H-pyrazole (4.2 g, 19.4 mmol), Na₂CO₃ (4.2 g, 38.8 mmol) and Pd(dppf)Cl₂ (0.4 g) under N₂. The mixture was heated to reflux and stirred under N₂ for 2 h. The mixture was cooled to at room temperature and filtered. The filtrate was poured into cold water, extracted with DCM/MeOH=10/1 for 10 times, the organic phase was washed with brine, dried over Na₂SO₄, and concentrated under vacuum to give crude solid. The crude solid was purified by silica gel chromatography to give S17-5 (3.5 g, 99.7%) as a yellow solid.

¹H NMR (CDCl₃, 400 MHZ): δ (ppm) 8.57 (d, J=2.4 Hz, 1H), 8.50 (d, J=2.4 Hz, 1H), 8.45 (s, 1H), 7.94 (s, 1H), 7.86 (s, 1H), 4.43 (q, J=7.2 Hz, 2H), 4.03 (s, 3H), 1.45 (t, J=7.2 Hz, 3H).

### 5. Synthesis of Intermediate Compound S17-6

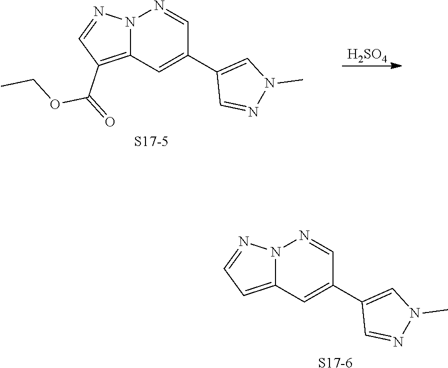 [SMILES: "S17-5" ; CCOC(=O)c1cnn2ncc(-c3cnn(C)c3)cc12 ; "S17-6" ; Cn1cc(-c2cnn3nccc3c2)cn1 ; "H2SO4"]

To a mixture of S17-5 (2.5 g, 9.2 mmol) in H2SO₄ (40% aq., 25 mL) was stirred at 100° C. overnight, poured into ice-water, extracted with DCM/MeOH=10/1 for 10 times, the organic phase was washed with brine, dried over Na₂SO₄, and concentrated under vacuum to give crude solid. The crude solid was triturated with MTBE to give S17-6 (1.6 g, 87.2%) as a yellow solid.

¹H NMR (DMSO-d₆, 400 MHZ): δ (ppm) 8.76 (s, 1H), 8.40-8.37 (m, 2H), 8.11 (s, 1H), 8.04 (s, 1H), 6.69 (d, J=2.0 Hz, 1H), 3.90 (s, 3H).

### 6. Synthesis of Compound S17

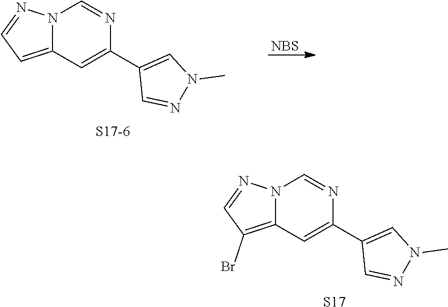

To a mixture of S17-6 (3.5 g, 17.6 mmol) in acetonitrile (70 mL) was added NBS (3.1 g, 17.6 mmol) in portions. The mixture was stirred at rt for 1 hr, poured into water, and then filtered. The filter cake was washed with water and dried to give S17 (2.5 g, 51%) as a yellow solid.

¹H NMR (CDCl₃, 400 MHz): δ (ppm) 8.86 (d, J=2.4 Hz, 1H), 8.53 (s, 1H), 8.21-8.26 (m, 3H), 3.91 (s, 3H).

### Synthesis of Compound S18

### 1. Synthesis of Intermediate Compound S18-2

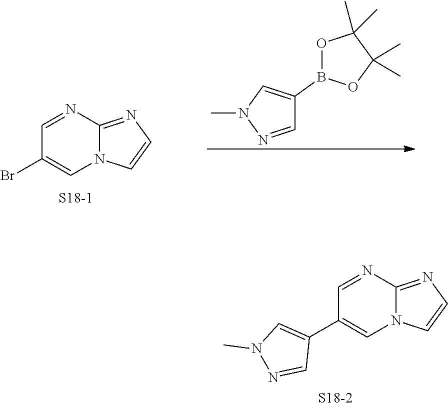

To a mixture of S18-1 (6.5 g, 32.8 mmol) in dioxane/water (130 mL/26 mL) was added 1-methyl-4-(4,4,5,5-tetramethyl-1,3,2-dioxaborolan-2-yl)-1H-pyrazole (10.2 g, 49.2 mmol), Na₂CO₃ (7.7 g, 72.2 mmol) and Pd(dppf)Cl₂ (1.3 g) under N₂. The mixture was heated to reflux and stirred under N₂ for 6 h. The mixture was cooled to at room temperature and filtered. The filtrate was poured into cold water, extracted with DCM/MeOH=10/1 for 10 times, the organic phase was washed with brine, dried over Na₂SO₄, and concentrated under vacuum to give crude solid. The crude solid was purified by silica gel chromatography to give S18-2 (5.6 g, 86.1%) as a yellow solid.

¹H NMR (DMSO-d₆, 400 MHZ): δ (ppm) 9.19 (d, J=2.0 Hz, 1H), 8.82 (d, J=2.4 Hz, 1H), 8.26 (s, 1H), 7.95 (s, 1H), 7.86 (s, 1H), 7.71 (d, J=0.8 Hz, 1H), 3.91 (s, 3H).

### 2. Synthesis of Compound S18

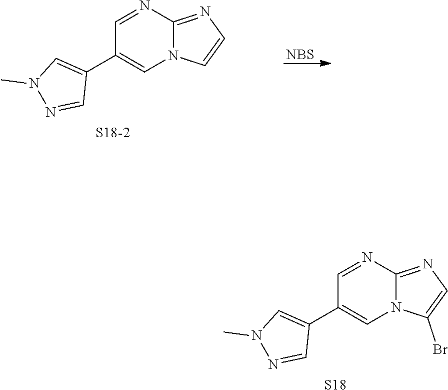

To a mixture of S18-2 (2.8 g, 14.1 mmol) in acetonitrile (60 mL) was added NBS (2.5 g, 14.1 mmol) in portions. The mixture was stirred at rt for 2 h, poured into water, and extracted with DCM/MeOH=10/1 for 10 times. The organic phase was washed with brine, dried over Na₂SO₄, and concentrated under vacuum to give crude solid. The crude solid was purified by silica gel chromatography to give S18 (2.0 g, 69.4%) as a yellow solid.

¹H NMR (DMSO-d₆, 400 MHZ): δ (ppm) 8.89-8.92 (m, 2H), 8.43 (s, 1H), 8.14 (s, 1H), 7.87 (s, 1H), 3.90 (s, 3H).

### Synthesis of Compound S19 (6-(1-methyl-1H-pyrazol-4-yl)-3-piperazin-1-ylpyrazolo[1,5-a]pyridine dihydrochloride)

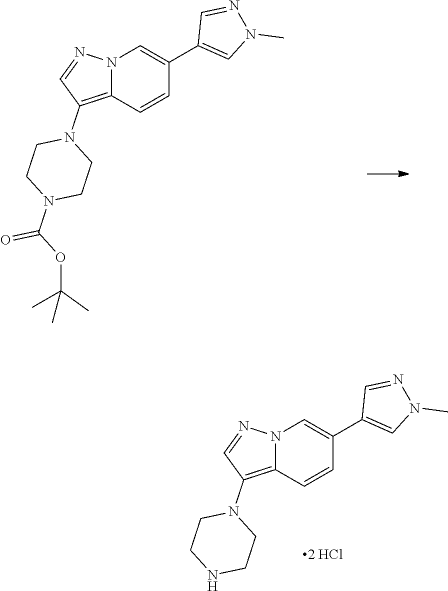 [SMILES: Cn1cc(-c2ccc3c(N4CCN(C(=O)OC(C)(C)C)CC4)cnn3c2)cn1 ; Cn1cc(-c2ccc3c(N4CCNCC4)cnn3c2)cn1]

To a round bottom flask containing tert-butyl 4-[6-(1-methyl-1H-pyrazol-4-yl)pyrazolo[1,5-a]pyridin-3-yl]piperazine-1-carboxylate (0.250 g, 0.65 mmol) was added methanol (5.00 mL) and 4M hydrogen chloride in 1,4-dioxane (2.00 mL, 8.00 mmol). The reaction was stirred at 27° C. for 24 hours, then stirred another 5 hours at 24° C. The mixture was filtered a through a frit, and the solid was rinsed with more methanol (5.00 mL). The solid was vacuum dried at 55° C. for 18 hours to obtain 227 mg (98%) of 6-(1-methyl-1H-pyrazol-4-yl)-3-piperazin-1-ylpyrazolo[1,5-a]pyridine dihydrochloride (S19) as a light gray solid. MS (ESI) m/z [M+H]⁺ calcd. for C₁₅H₂₁Cl₂N₆, 283.2; found, 283.2.

### Synthesis of Compound S20 (2-chloro-5-(3-methylbenzyl)pyrimidine)

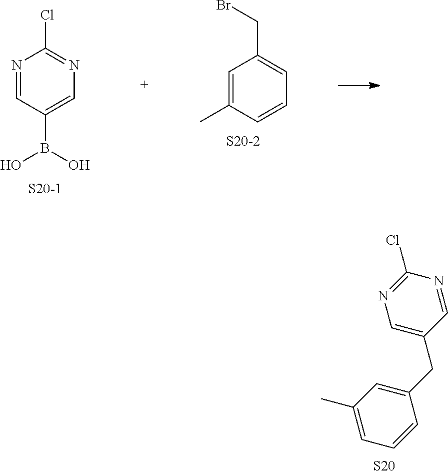

A suspension of (2-chloropyrimidin-5-yl) boronic acid (Compound S20-1, 3.50 g, 22.1 mmol), Na₂CO₃ (7.05 g, 66.5 mmol) and PdCl₂(PPh₃)₂ (783 mg, 1.12 mmol) in dioxane (17.5 mL) and water (7.2 mL) was sparged with argon for 15 min. To the suspension was added 1-bromomethyl-3-methylbenzene (Compound S20-2, 4.50 g, 24.3 mmol) and the suspension was sparged with argon for 10 min. The suspension was warmed in an oil bath at 100° C. under an argon atmosphere for 7.5 h. The heat was removed, and the reaction allowed to stand overnight. The reaction was diluted with EtOAc (150 mL) and filtered. The filter pad was washed with EtOAc (100 mL) and the filtrate washed with water (50 mL) and then brine (30 mL). The filtrate was dried (Na₂SO₄), filtered and concentrated. The residue was purified by ISCO chromatography (330 g normal phase cartridge) eluting with a gradient of 0-100% EtOAc in hexanes to afford 2.31 g, (48%) of 2-chloro-5-(3-methylbenzyl)pyrimidine (S20) as a yellow oil. MS (ESI) m/z [M+H]⁺ calcd. for C₁₂H₁₂ClN₂, 219.1; found, 219.0.

### Synthesis of Compound S21 (2-chloro-5-(3-methoxybenzyl)pyrimidine)

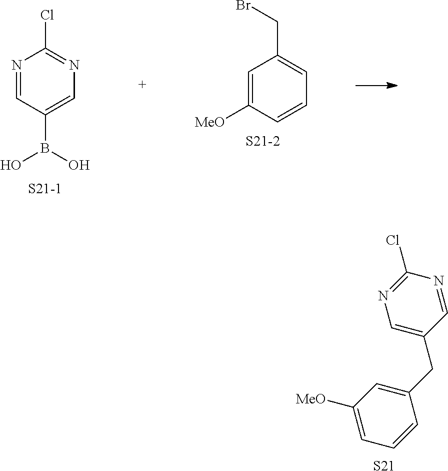 [SMILES: OB(O)c1cnc(Cl)nc1 ; COc1cccc(CBr)c1 ; "S21-1" ; COc1cccc(Cc2cnc(Cl)nc2)c1 ; "S21"]

A suspension of (2-chloropyrimidin-5-yl) boronic acid (Compound S21-1, 3.00 g, 18.9 mmol), Na₂CO₃ (6.04 g, 57.0 mmol) and PdCl₂(PPh₃)₂ (671 mg, 0.96 mmol) in dioxane (29.0 mL) and water (6.2 mL) was sparged with nitrogen for 15 min. To the suspension was added 3-methoxybenzyl bromide (Compound S21-2, 4.19 g, 20.8 mmol) and the suspension was sparged with nitrogen for 5 min. The suspension was warmed in an oil bath at 100° C. under a nitrogen atmosphere for an hour. The heat was removed, and the reaction allowed to cool to room temperature. The reaction was partitioned between water and EtOAc, and the organic phase was washed with water and brine and dried with Na₂SO₄. The residue was suspended in DCM and loaded onto a dry silica gel column and eluted with DCM to give 2.37 g (53%) of 2-chloro-5-(3-methoxybenzyl)pyrimidine (Compound S21) as an orange oil: 1H NMR (400 MHZ, CDCl₃) δ (ppm) 8.47 (s, 1H) 7.27 (m, 1H) 6.82 (dd, J=8.3, 2.3 Hz, 1H) 6.75 (d, J=7.5 Hz, 1H) 6.69 (t, J=1.9 Hz, 1H) 3.93 (s, 2H) 3.80 (s, 4H); MS (ESI) m/z [M+H]⁺ calcd. for C₁₂H₁₂ClN₂O, 235.1; found, 235.0; HPLC purity 210 nm: 100.0%; 254 nm: 100.0%.

### Synthesis of Compound S22 (2-[4-(3-piperazine-1-ylpyrazolo[1,5-a]pyridin-6-yl)-1H-pyrazol-1-yl]ethanol)

### 1. Synthesis of Intermediate Compound S22-2

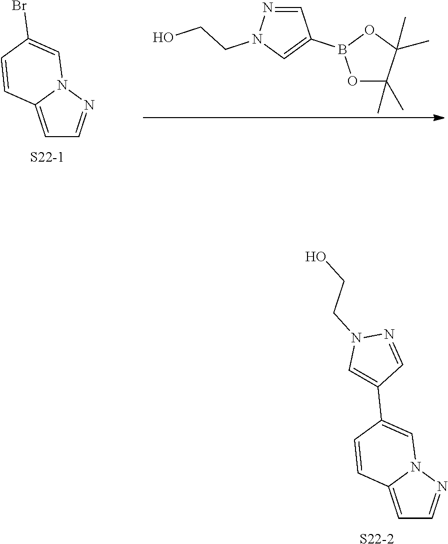 [SMILES: Brc1ccc2ccnn2c1 ; CC1(C)OB(c2cnn(CCO)c2)OC1(C)C ; "S22-1" ; OCCn1cc(-c2ccc3ccnn3c2)cn1 ; "S22-2"]

A solution of 6-bromopyrazolo[1,5-a]pyridine (Compound S22-1, 0.25 g, 1.27 mmol), and 2-[4-(4,4,5,5-tetramethyl-1,3,2-dioxaborolan-2-yl)-1H-pyrazol-1-yl]ethanol (0.33 g, 1.40 mmol) in dioxane (5.0 mL) and water (1.0 mL) was sparged with nitrogen for 20 minutes. Pd(PPh₃)₄ (37 mg, 0.03 mmol) and K₂CO₃ (351 mg, 2.54 mmol) were added and sparged with nitrogen for another 5 minutes. The yellow solution was heated in an oil bath at 100° C. under a nitrogen atmosphere for 2 hours. The heat was removed, and the reaction allowed to cool to room temperature. The reaction was partitioned between water and DCM and saturated aq. NaHCO₃ was added to clarify the organic phase. The organic phase was washed with aq. NaHCO₃ and dried with Na₂SO₄. The residue was suspended in DCM for chromatography, loaded onto a 12 g silica gel cartridge, and eluted with a gradient of acetone/DCM to give 197 mg (68%) of 2-(4-pyrazolo[1,5-a]pyridin-6-yl-1H-pyrazol-1-yl)ethanol (Compound S22-2) as a white solid: ¹H NMR (400 MHz, CDCl₃) δ (ppm) 8.63 (s, 1H) 7.94 (s, 1H) 7.80 (s, 1H) 7.72 (s, 1H) 7.56 (d, J=9.2 Hz, 1H) 7.24 (m, 1H) 6.52 (s, 1H) 4.32 (br. s., 2H) 4.08 (br. s., 2H) 3.16 (br. s., 1H); MS (ESI) m/z [M+H]⁺ calcd. for C₁₂H₁₃N₄O, 229.1; found, 229.0; HPLC purity 210 nm: 100.0%; 254 nm: 100.0%.

### 2. Synthesis of Intermediate Compound S22-3

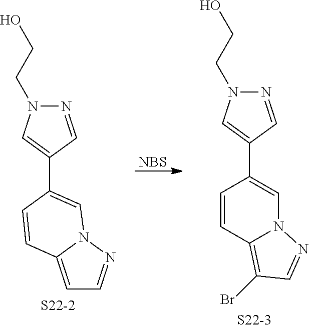

2-(4-pyrazolo[1,5-a]pyridin-6-yl-1H-pyrazol-1-yl)ethanol (Compound S22-2, 0.19 g, 0.83 mmol) was dissolved in DMF (2 mL), and the solution was stirred and cooled under a nitrogen atmosphere. N-Bromosuccinimide (0.18 g, 1.00 mmol) was added portion-wise at a rate to keep the temperature <10° C., and it became a paste. A solution of sodium thiosulfate (0.33 g, 2.08 mmol) and sodium bicarbonate (0.18 g, 2.08 mmol) in water (2 mL) was added and the mixture was stirred rigorously at room temperature for an hour. The solid was collected by filtration, washed with water (2 mL×4) and dried under vacuum to give to afford 256 mg (100%) of 2-[4-(3-bromopyrazolo[1,5-a]pyridin-6-yl)-1H-pyrazol-1-yl]ethanol (Compound S22-3) as a solid: MS (ESI) m/z [M+H]⁺ calcd. for C₁₂H₁₂BrN₄O, 307.0; found, 307.0; HPLC purity: 210 nm: 100.0%; 254 nm: 100.0%.

### 3. Synthesis of Intermediate Compound S22-4

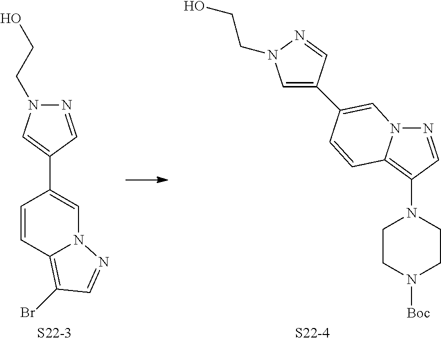

To a round bottom flask containing 2-[4-(3-bromopyrazolo[1,5-a]pyridin-6-yl)-1H-pyrazol-1-yl]ethanol (Compound S22-3, 0.25 g, 0.81 mmol) and tert-butyl-1-piperazinecarboxylic acid (0.30 g, 1.63 mmol) was added tert-butyl alcohol (6 mL) and 1,4-dioxane (3 mL). The solution was sparged with nitrogen for 10 min at 35° C., and then swept with argon atmosphere. To the solution was added sodium tert-butoxide (117 mg, 1.22 mmol) and the reaction mixture was swept with argon for 5 min. tBuXPhos Pd G1 (112 mg, 0.16 mmol) was added followed by a 5 min argon sweep. The flask was placed into a pre-heated 55° C. bath for 2 hr, cooled to ambient temperature and filtered through Celite® 545. The filtrate solvent was removed in vacuo to afford 0.54 g of tert-butyl 4-(6-[1-(2-hydroxyethyl)-1H-pyrazol-4-yl]pyrazolo[1,5-a]pyridin-3-yl)piperazine-1-carboxylate (Compound S22-4) as a brown solid unpurified mixture: LCMS (ESI) m/z [M+H]⁺ calcd. for C₂₁H₂₉N₆O₃, 413.2; found, 413.1; HPLC purity: 210 nm: 31.3%; 254 nm: 41.9%.

### 4. Synthesis of Compound S22

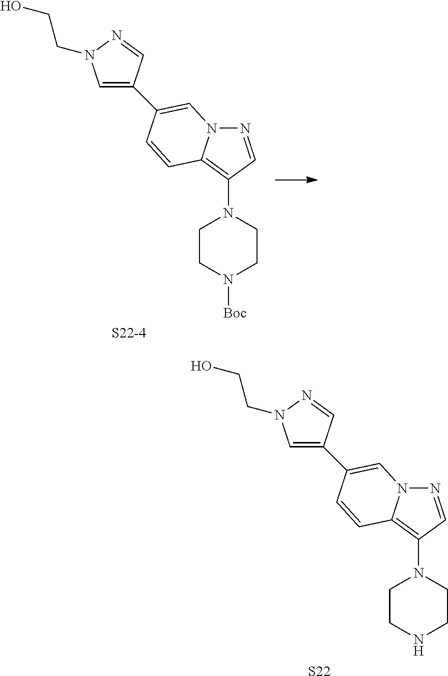 [SMILES: CC(C)(C)OC(=O)N1CCN(c2cnn3cc(-c4cnn(CCO)c4)ccc23)CC1 ; "S22-4" ; OCCn1cc(-c2ccc3c(N4CCNCC4)cnn3c2)cn1 ; "S22"]

To a round bottom flask containing tert-butyl 4-(6-[1-(2-hydroxyethyl)-1H-pyrazol-4-yl]pyrazolo[1,5-a]pyridin-3-yl)piperazine-1-carboxylate (Compound S22-4, 0.54 g) was added methanol (10 mL) and Dowex 50W4-200 resin (1.65 g washed and dried). The mixture was agitated and heated at 70° C. until no Compound S22-4 or Compound S22 were seen by HPLC or LCMS. The resin was filtered and washed with organic solvents, and the filtrates were discarded. Product was then removed from the resin with 3.5M NH₃ in MeOH, and solvent from this filtrate was removed in vacuo. The residue was dissolved in water and extracted with MTBE to remove remaining impurities. The aqueous phase was dried in vacuo to give solid that was in turn triturated with 10% 2-propanol in DCM. This organic triturate was dried in vacuo to afford 79 mg (31% for combined steps 3 and 4) of 2-[4-(3-piperazine-1-ylpyrazolo[1,5-a]pyridin-6-yl)-1H-pyrazol-1-yl]ethanol (Compound S22). ¹H NMR (400 MHz, CDCl₃, drop of MeOH-d4 added) δ ppm 8.40 (s, 1H) 7.76 (s, 1H) 7.72 (s, 1H) 7.63 (s, 1H) 7.47 (d, J=9.20 Hz, 1H) 7.12 (d, J=9.20 Hz, 1H) 4.21 (m, 2H) 3.91 (m, 2H) 3.35 (bs, 1H) 3.31 (s, 2.5H) 3.00 (br. s. 6.5H); MS (ESI) m/z [M+H]⁺ Calcd. for C₁₆H₂₁N₆: 283.3. Found: 313.1; HPLC purity: 210 nm: 93.1%; 254 nm: 90.4%.

### Synthesis of Compound S23 (2-Methoxy-5-phenyl-4,5-dihydro-1,3-oxazole)

### 1. Synthesis of Intermediate Compound S23-2

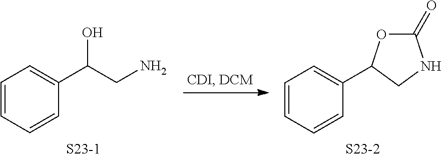

2-Amino-1-phenylethanol (Compound S23-1, 2.6 g, 16 mmol) was taken up in DCM (26 mL) at rt under argon and then cooled to 0° C. N,N-Carbonyldiimidazole (3.4 g, 21 mmol) was added in one portion (vigorous gas evolution). The reaction mixture was stirred at 0° C. for 30 min at which point HPLC indicated the complete disappearance of starting material. The reaction mixture was poured into a separatory funnel transferring with DCM (50 mL). The organic layer was washed sequentially with 1 M HCl (25 mL×2), saturated aq. sodium bicarbonate (25 mL), and brine (25 mL). The combined organic fractions were dried over sodium sulfate, filtered and the solvent removed in vacuo to afford a cream colored/pale yellow solid. The material was purified by Biotage Isolera chromatography using an Agela 120 g normal phase SG column eluting with a gradient of 100:0 to 9:1 DCM:MeOH. The desired fractions were combined and the solvent removed in vacuo to afford Compound S23-2 (2.0 g, 76% yield) as a white solid. MS (ESI) m/z [M+H]⁺ calcd. for C₉H₉NO₂: 163.1. Found: 164.0.

### 2. Synthesis of Compound S23

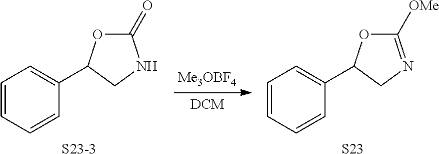

5-Phenyl-1,3-oxazolidin-2-one (Compound S23-2, 0.32 g, 2.0 mmol) was taken up in DCM (8 mL) at rt under argon. Trimethyloxonium tetrafluoroborate (1.5 g, 9.8 mmol) was then added to the mixture at rt and stirred at rt overnight. The reaction mixture was quenched by the addition of saturated aq. sodium bicarbonate (30 mL, 30 mmol) (gas evolution). The mixture was stirred at rt for 15 min then transferred to a separatory funnel. The mixture was extracted with DCM (30 mL×2). The combined organic fractions were dried over sodium sulfate, filtered and the solvent removed in vacuo to afford a pale yellow oil. The oil was re-subjected to trimethyloxonium tetrafluoroborate (1.5 g, 9.8 mmol) in DCM (8 mL) at rt overnight. The reaction mixture was quenched by the addition of saturated aq. sodium bicarbonate (30 mL, 30 mmol) (gas evolution). The mixture was stirred at rt for 15 min then transferred to a separatory funnel. The mixture was extracted with DCM (30 mL×2). The combined organic fractions were dried over sodium sulfate, filtered and the solvent removed in vacuo to afford Compound S23 (330 mg, 100% yield) as a pale yellow oil. MS (ESI) m/z [M+H]⁺ calcd. for C₁₀H₁₁NO₂: 177.2. Found: 178.0.

### Synthesis of Compound S24 (1-(4-benzyl-1,3-oxazol-2-yl])piperazine)

### 1. Synthesis of Intermediate Compound S24-3

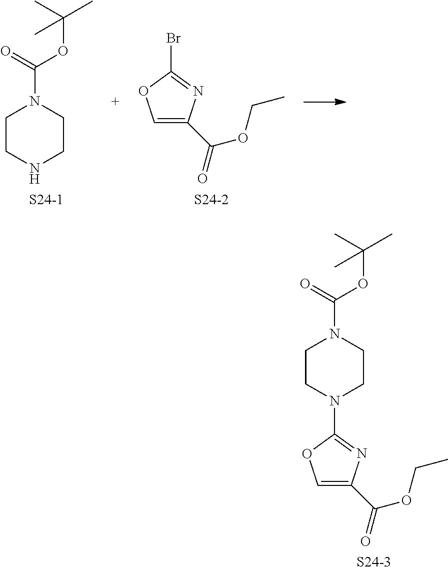 [SMILES: CC(C)(C)OC(=O)N1CCNCC1 ; CCOC(=O)c1coc(Br)n1 ; "S24-1" ; "S24-2" ; "S24-3" ; CCOC(=O)c1coc(N2CCN(C(=O)OC(C)(C)C)CC2)n1]

A suspension of ethyl 2-bromo-1,3-oxazole-4-carboxylatete (Compound S24-2, 0.500 g, 2.27 mmol), tert-butyl 1-piperazinecarboxylate (Compound S24-1, 0.510 g, 2.74 mmol), triethylamine (0.95 mL, 6.8 mmol) in dioxane (5.4 mL) in a 35 mL reaction vessel was microwaved for 1 h at 120° C. using a CEM microwave. The reaction was cooled to room temperature and concentrated. A second reaction was run in an identical manner using ethyl 2-bromo-1,3-oxazole-4-carboxylate (2.00 g, 9.09 mmol), tert-butyl 1-piperazinecarboxylate (2.04 g, 10.9 mmol), triethylamine (3.79 mL, 27.2 mmol) and dioxane (22 mL). The two crude reactions were combined and purified by ISCO chromatography (120 g normal phase cartridge) eluting with a gradient of 0-5% MeOH in DCM to afford 2.99 g, (81%-combined yield) of tert-butyl 4-[4-(ethoxycarbonyl)-1,3-oxazol-2-yl]piperazine-1-carboxylate (Compound S24-3) as a white waxy solid: MS (ESI) m/z [M+H]⁺ calcd. for C₁₅H₂₄N₃O₅, 326.2; found, 326.2.

### 2. Synthesis of Intermediate Compound S24-4

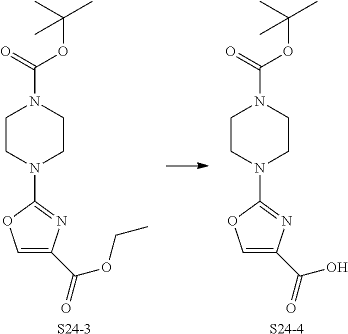 [SMILES: "S24-3" ; "S24-4" ; CCOC(=O)c1coc(N2CCN(C(=O)OC(C)(C)C)CC2)n1 ; CC(C)(C)OC(=O)N1CCN(c2nc(C(=O)O)co2)CC1]

To a solution of tert-butyl 4-[4-(ethoxycarbonyl)-1,3-oxazol-2-yl]piperazine-1-carboxylate (Compound S24-3, 4.00 g, 12.3 mmol) and THF (75.0 mL) was added MeOH (25.0 mL) and 1M NaOH (20.0 mL), 20.0 mmol). The solution was stirred at room temperature for 2.5 h, then concentrated in vacuo to remove the volatile organics. The aqueous residue was acidified to pH=4 with 1M HCl. The white suspension was diluted with DMF (50.0 mL) purified by ISCO chromatography (330 g reverse phase cartridge×2) eluting with a gradient of 0-100% ACN in water. Fractions of pure product were combined and concentrated to remove ACN. The aqueous residue was frozen and lyophilized to afford 3.37 g (92%) of 2-[4-(tert-butoxycarbonyl)piperazin-1-yl]-1,3-oxazole-4-carboxylic acid (Compound S24-4) as a white solid: MS (ESI) m/z [M+H]⁺ calcd. for C₁₃H₂₀N₃O₅, 298.1; found, 298.1.

### 3. Synthesis of Intermediate Compound S24-5

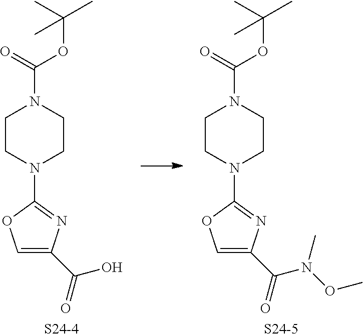

A solution of 2-[4-(tert-butoxycarbonyl)piperazin-1-yl]-1,3-oxazole-4-carboxylic acid (Compound S24-4, 2.11 g, 7.10 mmol) and DCM (55 mL) was cooled in an ice water bath. 1-hydroxybenzotriazole hydrate (1.41 g, 7.16 mmol), N,O-dimethylhydroxylamine hydrochloride (698 mg, 7.16 mmol), DIEA (1.25 mL, 7.16 mmol) and EDCl (1.37 g, 7.16 mmol) were added. The mixture was stirred for 45 min at 0° C. The ice bath was removed, and the reaction was allowed to come to room temperature. After stirring 5.5 h, additional 1-hydroxybenzotriazole hydrate (217 mg, 1.42 mmol), N,O-dimethylhydroxylamine hydrochloride (138 mg, 1.42 mmol), DIEA (0.25 mL, 1.42 mmol) and EDCl (272 mg, 1.42 mmol) was added to the reaction. After stirring overnight, the reaction was washed with saturated aq. NaHCO₃. The aqueous layer was washed with DCM (4×25 mL). The combined organics were dried (Na₂SO₄) filtered and concentrated. The residue was purified by ISCO chromatography (120 g normal phase cartridge) eluting with EtOAc to afford 2.08 g (86%) of tert-butyl 4-{4-[methoxy(methyl)carbamoyl]-1,3-oxazol-2-yl}piperazine-1-carboxylate (Compound S24-5) as a waxy white solid. MS (ESI) m/z [M+H]⁺ calcd. for C₁₅H₂₅N₄O₅, 341.2; found, 341.1.

### 4. Synthesis of Intermediate Compound S24-6

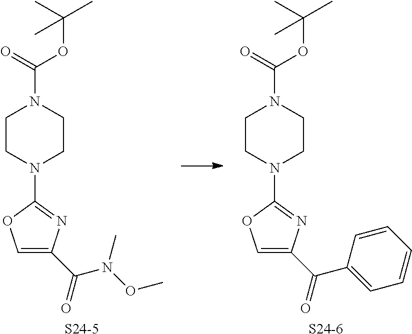

While under a nitrogen atmosphere, tert-butyl 4-{4-[methoxy(methyl)carbamoyl]-1,3-oxazol-2-yl}piperazine-1-carboxylate (Compound S24-5, 3.52 g, 10.3 mmol) was dissolved in anhydrous THF (44.0 mL) and cooled to −10° C. using a MeOH/ice bath. A 1.0 M solution of phenylmagnesium bromide in THF (23 mL, 23 mmol) was slowly added to the reaction to maintain the temperature at −10° C. (5 min). After stirring for 2.5 h−10° C., the reaction was then warmed to room temperature over 1.5 h before cooling to 0° C., and slowly quenching with saturated aq. NH₄Cl (23 ml). The quenched reaction was partitioned with EtOAc (233 mL) and water (80 mL). The aqueous layer was washed twice with EtOAc. The combined organic layers were washed with brine, dried (Na₂SO₄), filtered and concentrated. The residue was purified by ISCO chromatography (80 g normal phase cartridge) eluting with 0-50% EtOAc in hexane to afford 3.27 g (89%) of tert-butyl 4-(4-benzoyl-1,3-oxazol-2-yl)piperazine-1-carboxylate (Compound S24-6) as a white solid. MS (ESI) m/z [M+H]⁺ calcd. for C₁₉H₂₄N₃O₄, 358.2; found, 358.1.

### 5. Synthesis of Intermediate Compound S24-7

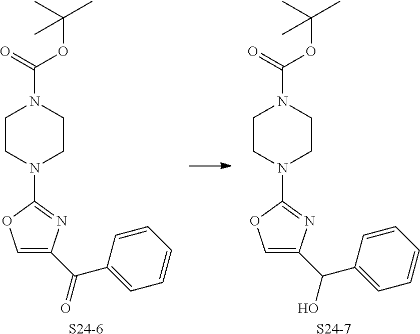

A suspension of tert-butyl 4-(4-benzoyl-1,3-oxazol-2-yl)piperazine-1-carboxylate (Compound S24-6, 0.99 g, 2.8 mmol) in MeOH (50 mL) was cooled in an ice bath. To the cold suspension was added NaBH₄ (0.21 g, 5.5 mmol). The ice bath was removed, and the reaction allowed to warm to room temperature. After stirring 1.5 h, the reaction was quenched with the cautious addition of 0.1 M HCl (40 mL). The quenched reaction was extracted with EtOAc (1×80 mL, 3×40 mL). The combined EtOAc layers were washed with saturated aq. NaHCO₃ (40 mL) and then saturated aq. NaCl (40 mL), dried (Na₂SO₄), filtered and concentrated. The large excess of white residue that was obtained was partitioned between DCM (80 mL) and water (80 ml). The DCM layer was washed with 0.1 M HCl (40 mL), then saturated aq. NaHCO₃, dried (Na₂SO₄), filtered and concentrated. The residue was stirred with hexane and concentrated several times to afford 0.94 g (94%) of tert-butyl 4-{4-[hydroxy(phenyl)methyl]-1,3-oxazol-2-yl]}piperazine-1-carboxylate (Compound S24-7) as a white solid. MS (ESI) m/z [M+H]⁺ calcd. for C₁₉H₂₆N₃O₄, 360.2; found, 360.1.

### 6. Synthesis of Intermediate Compound S24-8

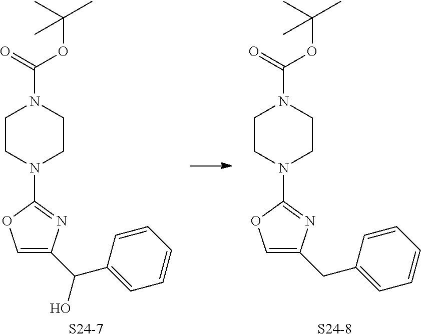

To a solution of tert-butyl 4-{4-[hydroxy(phenyl)methyl]-1,3-oxazol-2-yl]}piperazine-1-carboxylate (Compound S24-7, 929 mg, 2.58 mmol) and anhydrous DCM (9 mL) was added triethylsilane (7.6 mL, 48 mmol) followed by TFA (7.6 mL, 99 mmol). The reaction was stirred at room temperature overnight and then concentrated. The residue was concentrated several times with toluene to give a yellow oil. The crude residue was dissolved in DCM (18 mL). 1M NaOH (9.2 mL, 9.2 mmol) was added so that the pH of the aqueous layer was basic. To the biphasic reaction was added di-tert-butyldicarbonate (564 mg, 2.58 mmol) dissolved in DCM (9.2 mL). After 4 h, the two layers of the reaction were separated. The aqueous layer was extracted with DCM (×3). The combined DCM layers were washed with saturated aq. NaCl, dried (Na₂SO₄), filtered and concentrated. The residue was purified by ISCO chromatography (40 g normal phase cartridge) eluting with 0-85% EtOAc in hexane to afford 401 mg (45%) of tert-butyl 4-(4-benzyl-1,3-oxazol-2-yl])piperazine-1-carboxylate (Compound S24-8) as a white solid. MS (ESI) m/z [M+H]⁺ calcd. for C₁₉H₂₆N₃O₃, 344.2; found, 344.1.

### 7. Synthesis of Compound S24

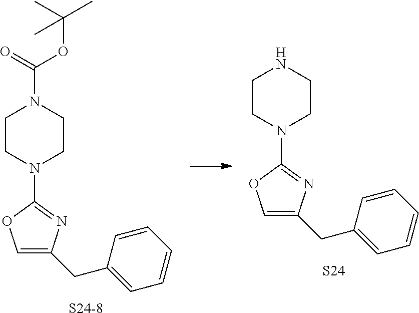

To a solution of tert-butyl 4-(4-benzyl-1,3-oxazol-2-yl])piperazine-1-carboxylate (Compound S24-8, 401 mg, 1.17 mmol) and MeOH (17 mL) was added freshly washed Dowex 50 WX 4-400 ion exchange resin (1.36 g). The suspension was gently stirred while warming at 50° C. After 1 h, the reaction was cooled to room temperature and filtered. The resin was washed with DCM (3×10 mL) then MeOH (3×10 mL). The resin was then washed with 3.5 M NH₃ in MeOH (5×10 mL) into a clean flask. The combined methanolic ammonia filtrates were concentrated to afford 275 mg (97%) of 1-(4-benzyl-1,3-oxazol-2-yl])piperazine (Compound S24) as a yellow oil. MS (ESI) m/z [M+H]⁺ calcd. for C₁₄H₁₈N₃O, 244.1; found, 240.0.

### Synthesis of Compound S25 ((2-chloropyrimidin-5-yl)(pyridin-2-yl)methanol)

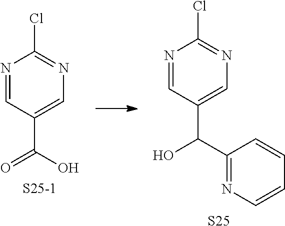 [SMILES: "S25-1" ; O=C(O)c1cnc(Cl)nc1 ; OC(c1cnc(Cl)nc1)c1ccccn1 ; "S25"]

1,2-Dibromoethane (1.10 mL, 12.8 mmol) and 2-bromopyridine (1.24 mL, 12.8 mmol) were added to a slurry of magnesium (0.620 g, 25.5 mmol) in dry THF (50 ml) under N₂ at rt. There was an exotherm and the mixture warmed itself to reflux. When the exotherm expired, the mixture was stirred at rt for 30 min, then cooled to 0° C. 2-chloropyrimidine-5-carbaldehyde (Compound S25-1, 2.00 g, 14.0 mmol) was added portion wise as a solid. The mixture was stirred at 0° C. for 2 hrs. LCMS showed no starting material. The reaction was quenched by addition of 20 ml of saturated NH₄Cl. The THF was evaporated and the organic products were extracted into 3×50 ml of EtOAc. The combined EtOAc layers were washed with 50 ml of brine and dried over Na₂SO₄. Evaporation of the solvent gave 2.3 g of a dark solid. Chromatography on an Isolera® in 50% EtAOc/CH₂Cl₂ to 100% EtOAc over 15 column volumes on 40 g of silica gel gave 1.3 g (46%) of (2-chloropyrimidin-5-yl) (pyridin-2-yl) methanol (Compound S25) as a light brown solid. ¹H NMR (300 MHZ, CDCl₃) δ (ppm) 8.60-8.71 (m, 3H), 7.71-7.84 (m, 1H), 7.32-7.34 (m, 1H), 7.19 (d, J=9 Hz, 1H), 5.82 (d, J=4.47 Hz, 1H), 5.22 (dd, J=4.75, 1.40 Hz, 1H); MS (ESI) m/z [M+H]⁺ calcd. for C₁₀H₈ClN₃O: 222.6; found: 223.9; HPLC purity: 210 nm; 100.0%; 254 nm: 100.0%.

### Synthesis of Compound S26 (cyclohexyl(2-{4-[6-(1-methyl-1H-pyrazol-4-yl)pyrazolo[1,5-a]pyridin-3-yl]piperazin-1-yl}pyrimidin-5-yl)methanone)

### 1. Synthesis of Intermediate Compound S26-2

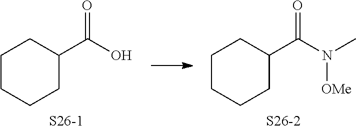

A solution of cyclohexanecarboxylic acid (Compound S26-1, 0.500 g, 3.90 mmol) and N,O-dimethylhydroxylamine hydrochloride (0.457 g, 4.68 mmol) in pyridine (2.6 mL) was treated with N-(3-dimethylaminopropyl)-N′-ethylcarbodiimide hydrochloride (0.897 g, 4.68 mmol) in two portions and the mixture was stirred at rt for 3 h. TLC in 5% MeOH/CH₂C₁₂ (stained with KI stain) showed no starting material. The reaction mixture was concentrated to a viscous yellow oil that was taken up in 40 mL H₂O and 10 mL brine. The solution was extracted with four 25 mL portions of EA and the combined organic phase was washed with 2×20 ml of 1.0N NaOH, 20 ml of water and 20 ml of brine. Drying over Na₂SO₄ and evaporation gave 0.44 g (66%) of N-methoxy-N-methylcyclohexanecarboxamide (Compound S26-2). ¹H NMR (400 MHZ, CDCl₃) δ (ppm) 3.71 (s, 3H), 3.19 (s, 3H), 2.70 (br. s., 1H) 1.73-1.86 (m, 4H), 1.64-1.73 (m, 1H), 1.43-1.59 (m, 2H) 1.21-1.37 (m, 3H); MS (ESI) m/z [M+H]⁺ calcd. for C₉H₁₇NO₂: 172.2; found: 172.1.

### 2. Synthesis of Intermediate Compound S26-4

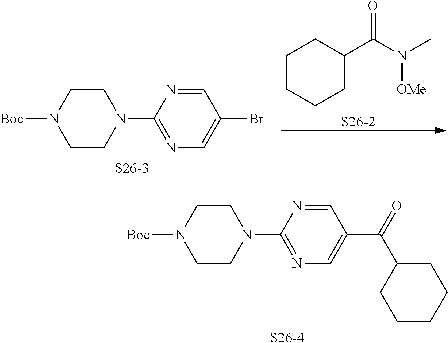

A solution of tert-butyl 4-(5-bromopyrimidin-2-yl)piperazine-1-carboxylate (Compound S26-3, 0.802 g, 2.34 mmol) in 10 ml of THF was cooled to −78° C. under N₂. n-Butyllithium in hexane (2.1 M, 1.22 mL, 2.57 mmol) was added at a rate to keep the temperature <−70° C. Addition took 5 minutes. When addition was complete, the mixture was stirred at −78° C. for 10 minutes. N-methoxy-N-methylcyclohexanecarboxamide (Compound S26-2, 0.400 g, 2.34 mmol) was added at a rate to keep the reaction temperature <−70° C. Addition took 5 minutes. The mixture was stirred at −78° C. for 1 half hr. HPLC (Agilent XDB C18 50×4.6 mm 1.8 micron column, Solvent A-Water (0.1% TFA) Solvent B-Acetonitrile (0.07% TFA). Gradient-5 min 95% A to 95% B; 1.0 min hold; then recycle. UV Detection @ 210 and 254 nm.) showed no starting bromide. LCMS showed mass for desired product minus t-butoxide. The reaction was quenched by addition of 10 ml of sat′d NH₄Cl and the mixture was warmed to rt. The THF was evaporated and the organic products were extracted into 3×10 ml of EtOAc. The combined EtOAc layers were washed with 10 ml of brine. Drying over Na₂SO₄ and evaporation gave 0.8 g of an off white solid. Chromatography on an Isolera® in 0% EtOAc/CH₂Cl₂ to 40% EtOAc/CH₂Cl₂ over 15 CV on 40 g of silica gel gave 0.2 g (23%) of tert-butyl 4-[5-(cyclohexylcarbonyl)pyrimidin-2-yl]piperazine-1-carboxylate (Compound S26-4) as a white solid ¹H NMR (300 MHz, CDCl₃) δ (ppm) 8.86 (s, 2H), 3.93 (d, J=5.40 Hz, 4H), 3.52 (d, J=5.12 Hz, 4H), 1.86 (d, J=9.78 Hz, 4H), 1.77 (br. s., 1H), 1.51 (s, 12H), 1.36 (br. s., 3H); MS (ESI) m/z [M+H]⁺ calcd. for C₂₀H₃₀N₄O₃: 375.5; found: 375.3; HPLC purity: 210 nm; 92.5%; 254 nm: 100%.

### 3. Synthesis of Intermediate Compound S26-5

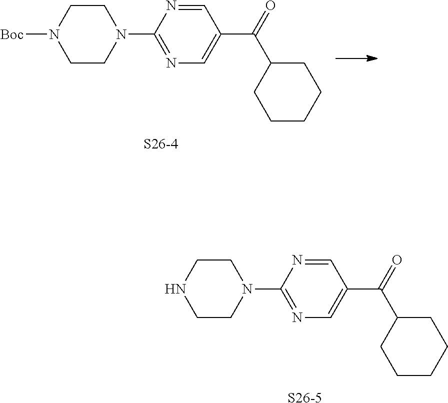 [SMILES: "S26-4" ; CC(C)(C)OC(=O)N1CCN(c2ncc(C(=O)C3CCCCC3)cn2)CC1 ; "S26-5" ; O=C(c1cnc(N2CCNCC2)nc1)C1CCCCC1]

Trifluoroacetic acid (0.206 mL, 2.67 mmol) was added to a solution of tert-butyl 4-[5-(cyclohexylcarbonyl)pyrimidin-2-yl]piperazine-1-carboxylate (Compound S26-4, 0.200 g, 0.534 mmol) in 10 ml of CH₂Cl₂ which was cooled in an ice water bath. The mixture was warmed to rt and stirred for 12 h. HPLC (Agilent XDB C18 50×4.6 mm 1.8 micron column, Solvent A-Water (0.1% TFA) Solvent B—Acetonitrile (0.07% TFA). Gradient-5 min 95% A to 95% B; 1.0 min hold; then recycle. UV Detection @ 210 and 254 nm.) showed no starting material. The CH₂Cl₂ was evaporated and the residue was dried under hi-vac at rt overnight. The residue was taken up in 10 ml of MeOH and treated with 10 eq of MP-carbonate resin at RT for 1 hr. The resin was removed by filtration and the filtrate was evaporated. Drying under hi-vac at rt overnight gave 0.12 g (82%) of cyclohexyl(2-piperazin-1-ylpyrimidin-5-yl) methanone (Compound S26-5) as a white solid. ¹H NMR (300 MHZ, CDCl₃) δ (ppm) 8.85 (s, 2H), 3.87-3.98 (m, 4H), 3.03 (m, 1H), 2.92-3.08 (m, 4H), 1.86 (d, J=9.69 Hz, 4H), 1.64 (br. s., 2H), 1.24-1.59 (m, 5H); MS (ESI) m/z [M+H]⁺ calcd. for C₁₅H₂₂N₄O: 275.4; found: 275.1; HPLC purity: 210 nm; 92.9%; 254 nm: 97.6%.

### 4. Synthesis of Compound S26

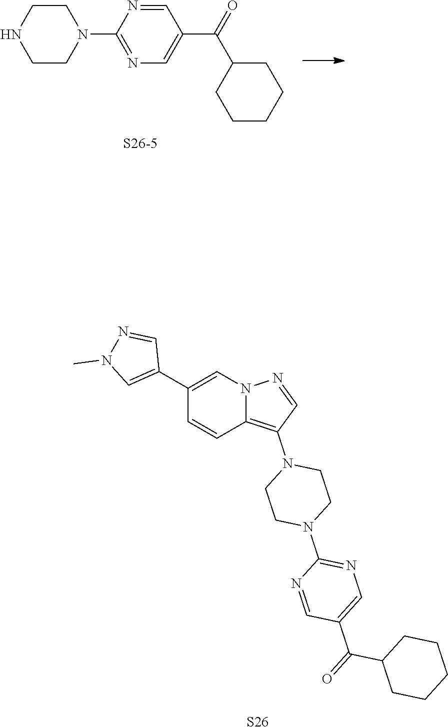 [SMILES: "S26-5" ; O=C(c1cnc(N2CCNCC2)nc1)C1CCCCC1 ; Cn1cc(-c2ccc3c(N4CCN(c5ncc(C(=O)C6CCCCC6)cn5)CC4)cnn3c2)cn1 ; "S26"]

A flask containing 3-bromo-6-(1-methyl-1H-pyrazol-4-yl)pyrazolo[1,5-a]pyridine (Compound S9, 0.101 g, 0.364 mmol) and cyclohexyl(2-piperazin-1-ylpyrimidin-5-yl) methanone (Compound S26-5, 0.120 g, 0.437 mmol) was treated with tert-butyl alcohol (0.906 mL) and 1,4-dioxane (0.228 mL). The solution was sparged with argon gas for 15 min. The solution was treated with tBuXPhos Pd G1 (0.038 g, 0.055 mmol) and sodium tert-butoxide (0.042 g, 0.44 mmol) and the mixture was sparged again for 10 min with argon gas then heated in a 55° C. oil bath overnight. LCMS showed no starting material. The mixture was cooled to rt and the catalyst was removed by filtration through Celite® 545. The filter cake was rinsed with 30 ml of CH₂Cl₂. The filtrate was extracted with 2×20 ml of sat′d NaHCO₃ and 20 ml of brine. Drying over Na₂SO₄ and evaporation gave 0.23 g of a dark oil. Chromatography on an Isolera® in 30% acetone/CH₂Cl₂ over 15 CV on 12 g of silica gel gave 0.09 g (52%) of cyclohexyl(2-{4-[6-(1-methyl-1H-pyrazol-4-yl)pyrazolo[1,5-a]pyridin-3-yl]piperazin-1-yl}pyrimidin-5-yl)methanone (Compound S26) as a light brown solid. ¹H NMR (300 MHz, CDCl₃) δ (ppm) 8.89 (d, J=1.40 Hz, 2H), 8.45-8.52 (m, 1H), 7.70-7.79 (m, 2H), 7.53-7.64 (m, 2H), 7.17 (d, J=9.22 Hz, 1H), 4.11-4.22 (m, 4H), 3.92-4.02 (m, 3H), 3.14 (t, J=4.75 Hz, 4H), 3.05 (br. s., 1H), 1.86 (d, J=11.27 Hz, 4H), 1.75 (d, J=11.64 Hz, 1H), 1.23-1.60 (m, 5H); MS (ESI) m/z [M+H]⁺ calcd. for C₂₆H₃₀N₈O: 471.6; found: 471.2; HPLC purity: 210 nm; 100.0%; 254 nm: 94.5%.

### Synthesis of Compound S27 (2-chloro-5-(4-chlorobenzyl)pyrimidine)

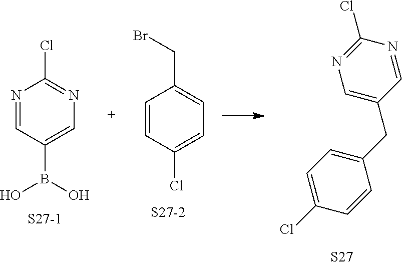 [SMILES: "S27-1" ; "S27-2" ; OB(O)c1cnc(Cl)nc1 ; Clc1ccc(CBr)cc1 ; Clc1ccc(Cc2cnc(Cl)nc2)cc1 ; "S27"]

A suspension of (2-chloropyrimidin-5-yl) boronic acid (S27-1, 3.50 g, 22.1 mmol), Na₂CO₃ (7.05 g, 66.5 mmol) and PdCl₂(PPh₃)₂ (783 mg, 1.12 mmol) in dioxane (17.5 mL) and water (7.2 mL) was sparged with argon for 15 min. To the suspension was added 1-(bromomethyl)-4-chlorobenzene (S27-2, 5.00 g, 24.3 mmol) and the suspension was sparged with argon for 10 min. The suspension was warmed in an oil bath at 100° C. under an argon atmosphere overnight. The heat was removed, and the reaction allowed to cool to room temperature. The reaction was diluted with EtOAc (110 mL) and filtered. The filter pad was washed with EtOAc (50 mL) and the filtrate washed with water (50 mL) and then brine (30 mL). The filtrate was dried (Na₂SO₄), filtered and concentrated. The residue was purified by ISCO chromatography (330 g normal phase cartridge) eluting with a gradient of 0-100% EtOAc in hexanes to afford 1.65 g, (31%) of 2-chloro-5-(4-chlorobenzyl)pyrimidine (Compound S27) as a yellow solid. MS (ESI) m/z [M+H]⁺ calcd. for C₁₁H₉Cl₂N₂, 239.0; found, 238.9.

### Synthesis of Compound S28 (2-chloro-5-(4-fluorobenzyl)pyrimidine)

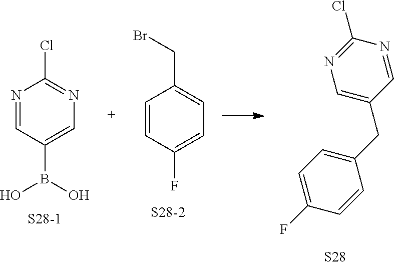 [SMILES: "S28-1" ; "S28-2" ; OB(O)c1cnc(Cl)nc1 ; Fc1ccc(CBr)cc1 ; Fc1ccc(Cc2cnc(Cl)nc2)cc1 ; "S28"]

A suspension of (2-chloropyrimidin-5-yl) boronic acid (Compound S28-1, 1.00 g, 6.32 mmol), Na₂CO₃ (2.01 g, 19.0 mmol) and PdCl₂(PPh₃)₂ (0.22 g, 0.32 mmol) in dioxane (5 mL) and water (2.0 mL) was sparged with argon for 15 min. To the suspension was added a-bromo-4-fluorotoluene (Compound S28-2, 0.86 mL, 6.95 mmol) and the suspension was sparged with argon for 10 min. The suspension was warmed in an oil bath at 100° C. under an argon atmosphere for 3 h. The heat was removed, and the reaction allowed to cool to room temperature. The reaction was diluted with EtOAc (30 mL) and filtered. The filter pad was washed with EtOAc (30 ml) and the filtrate washed with water (20 mL) and then brine (30 mL). The filtrate was dried (Na₂SO₄), filtered and concentrated. The residue was purified by ISCO chromatography (80 g normal phase cartridge) eluting with a gradient of 0-10% acetone in DCM to afford 0.47 g, (33%) of 2-chloro-5-(4-fluorobenzyl)pyrimidine (Compound S28) as an orange oil. MS (ESI) m/z [M+H]⁺ calcd. for C₁₁H₉ClFN₂, 223.0; found, 223.0.

### Synthesis of Compound S29 (2-chloro-5-(4-methylbenzyl)pyrimidine)

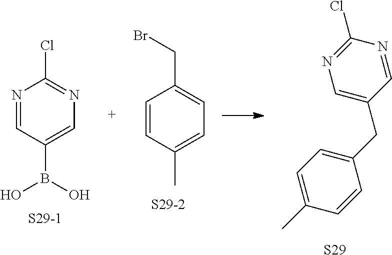

A suspension of (2-chloropyrimidin-5-yl) boronic acid (Compound S29-1, 5.00 g, 31.6 mmol), Na₂CO₃ (10.1 g, 94.9 mmol) and PdCl₂(PPh₃)₂ (1.12 g, 1.59 mmol) in dioxane (25.0 mL) and water (10.0 mL) was sparged with argon for 15 min. To the suspension was added 1-bromomethyl-4-methylbenzene (Compound S29-2, 6.43 g, 34.7 mmol) and the suspension was sparged with argon for 10 min. The suspension was warmed in an oil bath at 100° C. under an argon atmosphere for 10 h. The heat was removed, and the reaction allowed to cool to room temperature. The reaction was diluted with EtOAc (180 mL) and filtered. The filter pad was washed with EtOAc and the filtrate washed with water (80 mL) and then brine. The filtrate was dried (Na₂SO₄), filtered and concentrated. The residue was purified by ISCO chromatography (330 g normal phase cartridge) eluting with a gradient of 0-10% acetone in DCM to afford 3.11 g, (45%) of 2-chloro-5-(4-methylbenzyl)pyrimidine (Compound S29) as a yellow solid. MS (ESI) m/z [M+H]⁺ calcd. for C₁₂H₁₂ClN₂, 219.1; found, 219.0.

### Synthesis of Compound S30 (2-piperazin-1-yl-5-(tetrahydro-2H-pyran-4-ylmethyl)pyrimidine)

### 1. Synthesis of Intermediate Compound S30-2

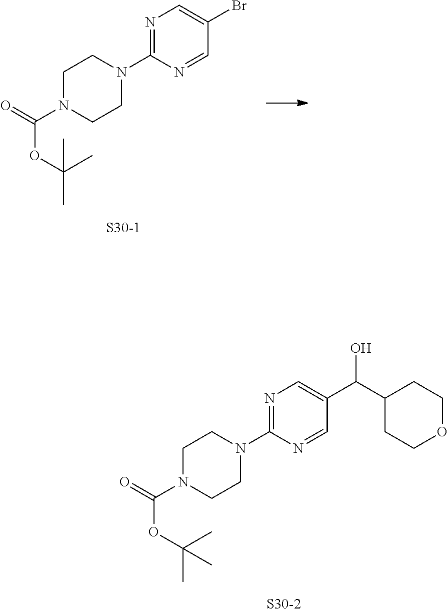 [SMILES: CC(C)(C)OC(=O)N1CCN(c2ncc(Br)cn2)CC1 ; CC(C)(C)OC(=O)N1CCN(c2ncc(C(O)C3CCOCC3)cn2)CC1]

To a solution of tert-butyl 4-(5-bromopyrimidin-2-yl)piperazine-1-carboxylate (Compound S30-1, 3.01 g, 8.76 mmol) in THF (40 mL) cooled to −78° C. was added 2.09 M n-BuLi in hexanes (4.6 mL, 9.6 mmol) over 12 min keeping the temperature <−65° C. The reaction was stirred for 5 min. To the cold reaction was added a solution of tetrahydro-2H-pyran-4-carbaldehyde (1.00 g, 8.76 mmol) dissolved in THF (4.0 ml) over 5 min keeping the temperature <−65° C. The dry ice bath was removed, and the reaction was allowed to warm to room temperature. After 1.5 h, the reaction was cooled to 0° C., and quenched with saturated aq. NH₄Cl (20 mL). To the reaction was added EtOAc (20 mL) and enough water to dissolve any solids. The quenched reaction was partitioned, and the aqueous layer washed with EtOAc (3×20 mL). The organic layers were combined, dried (Na₂SO₄), filtered and concentrated. The residue was purified by ISCO chromatography (120 g cartridge) eluting with a gradient of EtOAc in hexanes to afford 2.82 g (69%) of 4-{5-[hydroxy(tetrahydro-2H-pyran-4-yl)methyl]pyrimidin-2-yl}piperazine-1-carboxylate (Compound S30-2) as a tan solid: MS (ESI) m/z [M+H]⁺ calcd. for C₁₉H₃₁N₄O₄, 379.2; found, 379.3.

### 2. Synthesis of Intermediate Compound S30-3

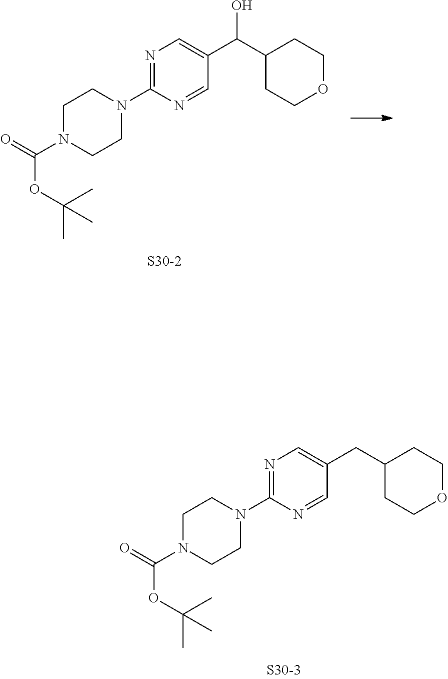 [SMILES: CC(C)(C)OC(=O)N1CCN(c2ncc(C(O)C3CCOCC3)cn2)CC1 ; CC(C)(C)OC(=O)N1CCN(c2ncc(CC3CCOCC3)cn2)CC1]

To a solution of 4-{5-[hydroxy(tetrahydro-2H-pyran-4-yl)methyl]pyrimidin-2-yl}piperazine-1-carboxylate (Compound S30-2, 2.24 g, 5.92 mmol) dissolved in anhydrous DCM (20 mL) was added triethylsilane (17.0 ml, 110 mmol) followed by trifluoroacetic acid (17.0 mL, 220 mmol). After stirring for 2-3 days, the volatiles were removed under reduced pressure. The residue was concentrated five times from toluene. The crude was dissolved in DCM (20 mL). Added 1M NaOH (21 mL, 21 mmol) so that the pH of the aqueous layer was basic. To the biphasic reaction was added a solution of di-tert-butyldicarbonate (1.29 g, 5.92 mmol) dissolved in DCM (20 mL). After stirring 3h at room temperature the biphasic reaction layers were separated. The aqueous layer was washed with DCM (3×20 mL). The combined DCM layers were washed with saturated aq. NaCl (20 mL), dried (Na₂SO₄), filtered and concentrated. The residue was purified by ISCO chromatography (120 g normal phase cartridge) eluting with a linear gradient to 25% ethyl acetate in hexanes to afford 1.44 g (67%) of tert-butyl 4-[5-(tetrahydro-2H-pyran-4-ylmethyl)pyrimidin-2-yl]piperazine-1-carboxylate (Compound S30-3) as a white solid: MS (ESI) m/z [M+H]⁺ calcd. for C₁₉H₃₁N₄O₃, 363.2; found, 363.3.

### 3. Synthesis of Compound S30

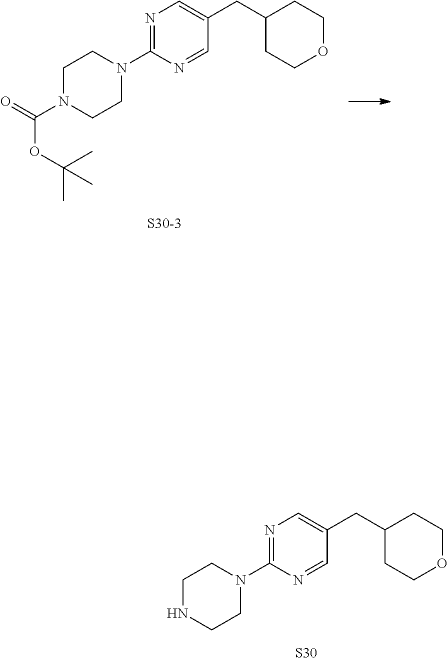 [SMILES: CC(C)(C)OC(=O)N1CCN(c2ncc(CC3CCOCC3)cn2)CC1 ; c1nc(N2CCNCC2)ncc1CC1CCOCC1 ; "S30"]

To a solution of tert-butyl 4-[5-(tetrahydro-2H-pyran-4-ylmethyl)pyrimidin-2-yl]piperazine-1-carboxylate (Compound S30-3, 1.33 g, 3.67 mmol) dissolved in MeOH (50 mL) was added freshly washed and dried Dowex 50WX4-400 ion exchange resin (4 g). The suspension was gently stirred and warmed to 50° C. After stirring 4 h, the reaction was cooled to room temperature and the suspension filtered. The resin was washed with MeOH (5 mL), then DCM (5 mL) and finally MeOH (5 mL). The resin was then washed with 3.5 M NH₃ in MeOH (3×15 mL). The methanolic ammonia filtrate was concentrated to afford 0.91 g (90%) of 2-piperazin-1-yl-5-(tetrahydro-2H-pyran-4-ylmethyl)pyrimidine (Compound S30) as a tan colored solid: MS (ESI) m/z [M+H]⁺ calcd. for C₁₄H₂₃N₄O, 263.2; found, 263.2.

### Synthesis of Compound S31 ((2-{4-[6-(1-methyl-1H-pyrazol-4-yl)pyrazolo[1,5-a]pyridin-3-yl]piperazin-1-yl}pyrimidin-5-yl)(tetrahydro-2H-pyran-4-yl)methanone)

### 1. Synthesis of Intermediate Compound S31-2

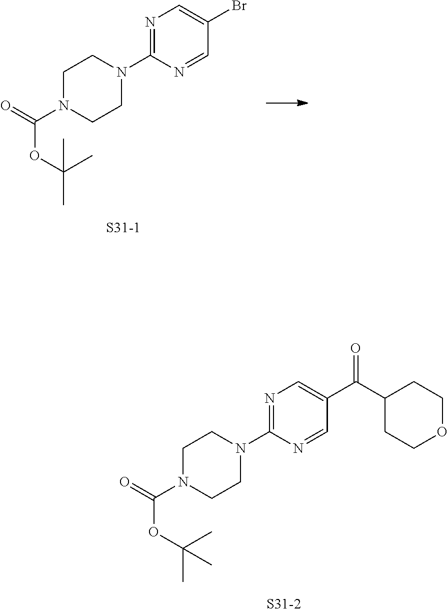 [SMILES: CC(C)(C)OC(=O)N1CCN(c2ncc(Br)cn2)CC1 ; "S31-1" ; CC(C)(C)OC(=O)N1CCN(c2ncc(C(=O)C3CCOCC3)cn2)CC1]

To a solution of tert-butyl 4-(5-bromopyrimidin-2-yl)piperazine-1-carboxylate (Compound S31-1, 3.01 g, 8.76 mmol) in THF (40 mL) cooled to −78° C. was added 2.09 M n-BuLi in hexanes (4.6 mL, 9.6 mmol) over 12 min keeping the temperature <−65° C. The reaction was stirred for 5 min. To the cold reaction was added a solution of N-methoxy-N-methyltetrahydro-2H-pyran-4-carboxamide (1.52 g, 8.76 mmol) dissolved in THF (4.0 ml) over 5 min keeping the temperature <−70° C. The dry ice bath was removed, and the reaction was allowed to warm to room temperature. After 4.5 h, the reaction was cooled to 0° C., and quenched with saturated aq. NH₄Cl (35 mL). Stirred 30 min. To the reaction was added EtOAc (35 mL). The ice bath was removed and the reaction allowed to warm to room temperature. Additional water (35 mL) was added to dissolve any solids. The EtOAc was removed in vacuo and replaced with DCM (35 mL). The quenched reaction was partitioned, and the aqueous layer washed with DCM (2×35 mL). The organic layers were combined, dried (Na₂SO₄), filtered and concentrated. The residue was purified by ISCO chromatography (330 g cartridge) eluting with a gradient of EtOAc in hexanes to afford 1.53 g (47%) of tert-butyl 4-[5-(tetrahydro-2H-pyran-4-ylcarbonyl)pyrimidin-2-yl]piperazine-1-carboxylate (Compound S31-2) as a white solid: MS (ESI) m/z [M+H]⁺ calcd. for C₁₉H₂₉N₄O₄, 377.2; found, 377.3.

### 2. Synthesis of Intermediate Compound S31-3

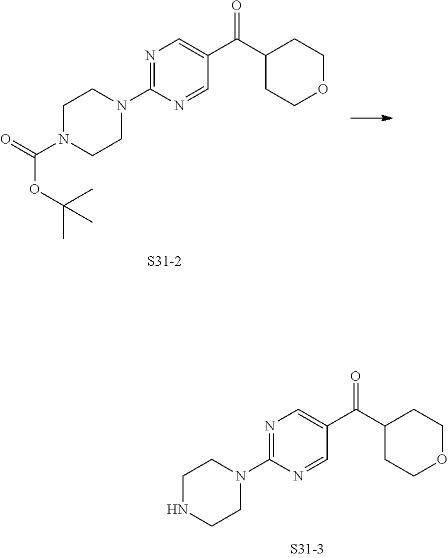 [SMILES: CC(C)(C)OC(=O)N1CCN(c2ncc(C(=O)C3CCOCC3)cn2)CC1 ; O=C(c1cnc(N2CCNCC2)nc1)C1CCOCC1]

A solution of tert-butyl 4-[5-(tetrahydro-2H-pyran-4-ylcarbonyl)pyrimidin-2-yl]piperazine-1-carboxylate (Compound S31-2, 1.31 g, 3.48 mmol) dissolved in anhydrous DCM (40 mL) was cooled in an ice bath. Added TFA (5.36 mL, 69.6 mmol). After stirring cold for 2.75 h, the volatiles were removed under reduced pressure. The residue was concentrated four times from toluene. The crude was dissolved in MeOH (100 mL). Added DIEA (0.06 mL, 35 mmol) and 5.6 g of polymer bound tetraalkylammonium carbonate resin (2.5-3.5 mmol/g). After stirring 1h at room temperature, the reaction was filtered, and the resin washed with MeOH (3×50 mL). The filtrate was concentrated. The residue was redissolved in MeOH (100 mL). Added DIEA (0.06 mL) and 5.6 g of polymer bound tetraalkylammonium carbonate resin (2.5-3.5 mmol/g). After stirring 2h, another 5.6 g of polymer bound tetraalkylammonium carbonate resin (2.5-3.5 mmol/g) was added. After 1 h, the reaction was filtered, and the resin washed with MeOH (3×50 mL). The filtrate was concentrated to afford 0.90 g (93%) of (2-piperazin-1-ylpyrimidin-5-yl)(tetrahydro-2H-pyran-4-yl)methanone (Compound S31-3) as a tan colored solid: MS (ESI) m/z [M+H]⁺ calcd. for C₁₄H₂₀N₄O₂, 277.2; found, 277.2.

### 3. Synthesis of Compound S31

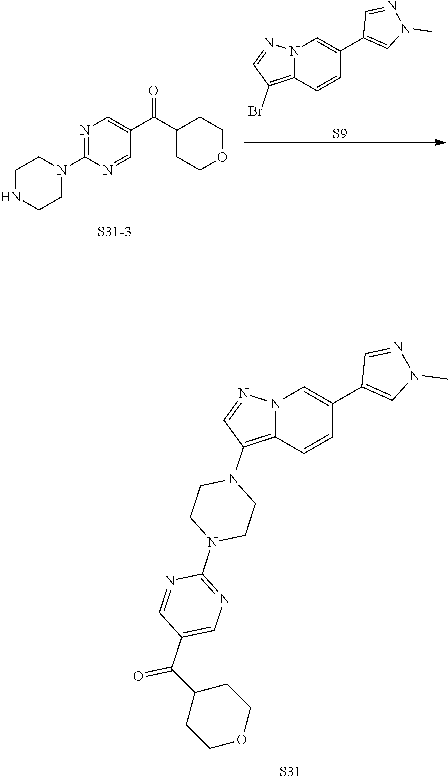

To a vial 3-bromo-6-(1-methyl-1H-pyrazol-4-yl)pyrazolo[1,5-a]pyridine (Compound S9, 0.20 g, 0.73 mmol) and (2-piperazin-1-ylpyrimidin-5-yl)(tetrahydro-2H-pyran-4-yl)methanone (Compound S31-3, 0.24 g, 0.87 mmol) was added tert-butyl alcohol (5.4 mL) and 1,4-dioxane (1.5 mL). The partially dissolved mixture was sparged with argon for 5 min. To the mixture was added sodium tert-butoxide (105 mg, 1.09 mmol). The mixture was sparged again for 5 min with argon and tBuXPhos Pd G1 (75 mg, 0.11 mmol) added. The reaction mixture was sparged with argon for 5 min and an additional 2 min with sonication. The flask was heated in a 55° C. oil bath. After stirring 21 h, the reaction cooled to ambient temperature and diluted with EtOAc (20 mL). The mixture was washed with saturated aq. NaHCO₃ (20 mL). The aqueous layer was washed with EtOAc (2×20 mL). The aqueous layer was diluted with water (10 mL) and satd. aq. NaCl (5 mL) and washed with EtOAc (2×25 mL). The combined EtOAc layers were dried (Na₂SO₄), filtered and concentrated. The residue was purified by ISOC chromatography (40 g normal phase cartridge) eluting with a gradient of 0-80% acetone in DCM to afford 151 mg, (44%) of (2-{4-[6-(1-methyl-1H-pyrazol-4-yl)pyrazolo[1,5-a]pyridin-3-yl]piperazin-1-yl}pyrimidin-5-yl)(tetrahydro-2H-pyran-4-yl)methanone (Compound S31); MS (ESI) m/z [M+H]⁺ calcd. for C₂₅H₂₉N₈O₂, 473.2; found, 473.3.

### Synthesis of Compound S32 (2-benzyl-5-piperazin-1-ylpyrimidine)

### 1. Synthesis of Intermediate Compound S32-2

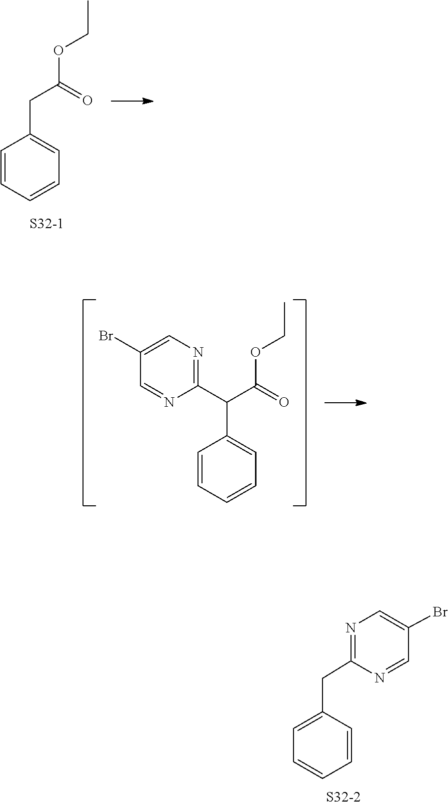

LDA was prepared by addition of 2.10 M of n-butyllithium in hexane (13.1 mL, 27.6 mmol) to a solution of N,N-diisopropylamine (3.86 mL, 27.6 mmol) in 40 ml of THF which was cooled to −78° C. When addition was complete the mixture was warmed to 0° C., and stirred at 0° C. for 15 min. A solution of benzeneacetic acid, ethyl ester (Compound S32-1, 4.28 mL, 26.9 mmol) in 5 ml of THF was added slowly to keep the temperature≤3° C. (addition took 20 min) and the mixture was stirred at 0° C. for 15 min. A solution of 5-bromo-2-chloropyrimidine (2.60 g, 13.4 mmol) in 10 ml of THF was added and the mixture was stirred at 0° C. for 30 minutes. The ice bath was removed and the mixture was warmed to rt. The mixture was stirred at rt overnight. TLC in 10% EtOAc/hexane showed no starting material. The reaction mixture was poured into 50 ml of ice cooled 1.0N HCl. The THF was evaporated and the mixture was extracted with 3×30 ml of EtOAc. The combined EtOAc layers were extracted with 50 ml of brine. Drying over Na₂SO₄ and evaporation gave 7 g of a red oil. The crude ester was purified by chromatography on an Isolera® in 100% hexane (2 CV) followed by 10% EtOAc/hexane for 15 CV on 120 g of silica gel. Yielded 1.6 g of a yellow oil. NMR, MS showed mixture of ethyl (5-bromopyrimidin-2-yl) (phenyl)acetate and 2-benzyl-5-bromopyrimidine. The oil was taken up in 30 ml of MeOH and treated with 10 ml (2.1 eq based on oil isolated) of 1.0N NaOH+10 ml of water. The mixture was stirred at rt for 4 hrs. HPLC (Agilent XDB C18 50×4.6 mm 1.8 micron column, Solvent A—Water (0.1% TFA) Solvent B—Acetonitrile (0.07% TFA). Gradient—5 min 95% A to 95% B; 1.0 min hold; then recycle. UV Detection @ 210 and 254 nm) showed no starting material. The mixture was acidified to pH˜4 by addition of 11 ml (2.2 eq based on oil isolated) of 1.0N HCl. The MeOH was evaporated and the mixture was extracted with 3×20 ml of EtOAc. The combined EtOAc layers were extracted with 20 ml of brine and dried over Na₂SO₄. Evaporation gave 1.2 g of a green solid. The solid was taken up in 20 ml of THF and heated in a 60° C. oil bath. After 1 hr, HPLC showed no starting material. The mixture was cooled to rt and the THF was evaporated to give 1.1 g of a brown solid. Chromatography on an Isolera® in CH₂Cl₂ on 12 g of silica gel gave 0.43 g (12%) of 2-benzyl-5-bromopyrimidine (Compound S32-2) as a white solid. ¹H NMR (300 MHZ, CDCl₃) δ (ppm) 8.73 (s, 2H), 7.29-7.40 (m, 4H), 7.26 (d, J=6.24 Hz, 1H), 4.27 (s, 2H); MS (ESI) m/z [M+H]⁺ calcd. for: C₁₁H₁₀BrN₂: 250.11; found: 250.9; HPLC purity: 210 nm: 95.9%; 254 nm: 100%.

### 2. Synthesis of Intermediate Compound S32-3

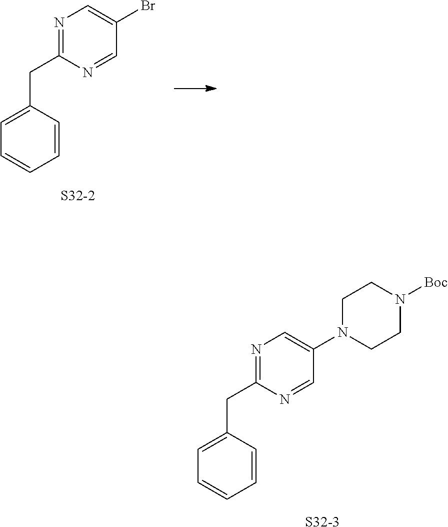 [SMILES: Brc1cnc(Cc2ccccc2)nc1 ; "S32-2" ; CC(C)(C)OC(=O)N1CCN(c2cnc(Cc3ccccc3)nc2)CC1 ; "S32-3"]

A flask containing 2-benzyl-5-bromopyrimidine (Compound S32-2, 0.200 g, 0.803 mmol and tert-butyl 1-piperazinecarboxylate (0.179 g, 0.963 mmol) was treated with tert-butyl alcohol (2.00 mL, 20.9 mmol) and 1,4-dioxane (0.501 mL, 6.42 mmol). The solution was sparged with argon gas for 15 min. The solution was treated with tBuXPhos Pd G1 (0.0827 g, 0.120 mmol) and sodium tert-butoxide (0.093 g, 0.96 mmol) and the mixture was sparged again for 10 min with argon gas then heated in a 55° C. heating block. After 5 hrs, LCMS showed no starting material. The mixture was cooled to rt and the catalyst was removed by filtration through Celite 545®. The filter cake was rinsed with 20 ml of CH₂Cl₂. The filtrate was extracted with 2×20 ml of saturated NaHCO₃ and 20 ml of brine. Drying over Na₂SO₄ and evaporation gave 0.4 g of a dark oil. Chromatography on an Isolera® in 0% acetone/CH₂Cl₂ to 60% acetone/CH₂Cl₂ over 15 CV on 20 g of silica gel gave 0.23 g (80%) of tert-butyl 4-(2-benzylpyrimidin-5-yl)piperazine-1-carboxylate (Compound S32-3) as a light brown solid. ¹H NMR (300 MHZ, CDCl₃) δ (ppm) 8.33 (s, 2H), 7.19-7.38 (m, 6H), 4.23 (s, 2H), 3.57-3.64 (m, 4H), 3.11-3.21 (m, 4H), 1.49 (s, 9H); MS (ESI) m/z [M+H]⁺ calcd. for C₂₀H₂₇N₄O₂: 355.4; found: 355.2; HPLC purity: 210 nm; 100%; 254 nm: 100%.

### 3. Synthesis of Compound S32

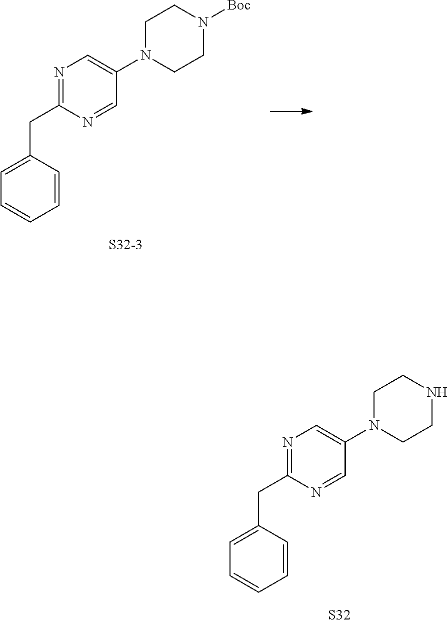 [SMILES: CC(C)(C)OC(=O)N1CCN(c2cnc(Cc3ccccc3)nc2)CC1 ; "S32-3" ; c1ccc(Cc2ncc(N3CCNCC3)cn2)cc1 ; "S32"]

Trifluoroacetic Acid (0.500 mL, 6.49 mmol) was added to a solution of tert-butyl 4-(2-benzylpyrimidin-5-yl)piperazine-1-carboxylate (Compound S32-3, 0.230 g, 0.65 mmol) in 10 ml of CH₂Cl₂ which was cooled in an ice water bath. The mixture was warmed to rt and stirred overnight. HPLC (Agilent XDB C18 50×4.6 mm 1.8 micron column, Solvent A—Water (0.1% TFA) Solvent B-Acetonitrile (0.07% TFA). Gradient-5 min 95% A to 95% B; 1.0 min hold; then recycle. UV Detection@210 and 254 nm.) showed no starting material. The CH₂Cl₂ was evaporated and the residue was dried under hi-vac at rt for 6 hrs. The remaining oil was taken up in 10 ml of MeOH and stirred with MP-carbonate resin (10 eq, 2.3 g) overnight at rt. The resin was removed by filtration through Celite-545® and the filter cake was washed with 10 ml of MeOH. Evaporation of the filtrate and drying under hi-vac at rt overnight gave 0.09 g of 2-benzyl-5-piperazin-1-ylpyrimidine (Compound S32). ¹H NMR (400 MHz, CDCl₃) δ (ppm) 8.33 (s, 2H), 7.25-7.43 (m, 4H), 7.13-7.25 (m, 1H), 4.23 (s, 2H), 3.11-3.25 (m, 4H), 2.96-3.11 (m, 4H); MS (ESI) m/z [M+H]⁺ calcd. for C₁₅H₁₉N₄: 255.3; found: 255.1; HPLC purity: 210 nm; 96.4%; 254 nm: 97.7%.

### Synthesis of Compound S33 ((2-{4-[6-(1-methyl-1H-pyrazol-4-yl)pyrazolo[1,5-a]pyridin-3-yl]piperazin-1-yl}pyrimidin-5-yl)(phenyl)methanone)

### 1. Synthesis of Intermediate Compound S33-1

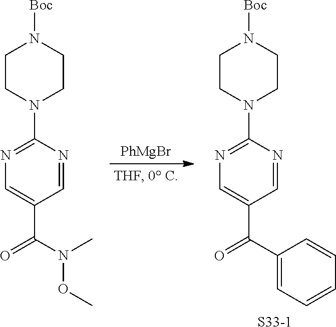 [SMILES: "PhMgBr" ; CON(C)C(=O)c1cnc(N2CCN(C(=O)OC(C)(C)C)CC2)nc1 ; CC(C)(C)OC(=O)N1CCN(c2ncc(C(=O)c3ccccc3)cn2)CC1 ; "S33-1"]

tert-Butyl 4-{5-[methoxy(methyl)carbamoyl]pyrimidin-2-yl}piperazine-1-carboxylate (see Example 28, steps 1-3; 1.72 g, 4.89 mmol) was taken up in THF (10 mL) in a 100 mL round bottom flask and cooled to 0° C. (ice/water bath). 1.0 M of phenylmagnesium bromide in THF (10.8 mL, 10.8 mmol) was added over a period of 5 minutes. The reaction was stirred at 0° C., and then quenched by the slow addition of 1 M HCl (6.50 mL, 6.50 mmol). The reaction mixture was poured into a separatory funnel transferring with EtOAc. The aqueous layer was removed and the pH adjusted to 1-2 by the addition of more 1M HCl. The aqueous layer poured back into the separatory funnel and the mixture was extracted with ethyl acetate (30 mL×2). The combined organic layers were washed with brine (30 mL) and then dried over sodium sulfate, filtered and the solvent removed in vacuo to afford Compound S33-1 as an off-white solid. The material was not purified but used as is for the next step. MS (ESI) m/z [M+H]⁺ calcd. for C₂₀H₂₅N₄O₃: 3698.2. Found: 369.2.

### 2. Synthesis of Intermediate Compound S33-2

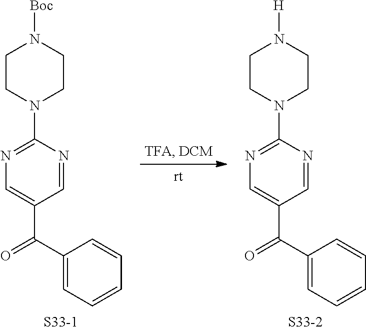 [SMILES: CC(C)(C)OC(=O)N1CCN(c2ncc(C(=O)c3ccccc3)cn2)CC1 ; O=C(c1ccccc1)c1cnc(N2CCNCC2)nc1 ; "S33-1" ; "S33-2"]

tert-Butyl 4-(5-benzoylpyrimidin-2-yl)piperazine-1-carboxylate (Compound S33-1, 1.80 g, 4.88 mmol) was taken up in DCM (20 mL) under argon. Trifluoroacetic acid (4.0 mL, 50 mmol) was added and the reaction mixture stirred at rt overnight. The reaction mixture was poured into a separatory funnel transferring with DCM (50 mL) and saturated aq. sodium bicarbonate (20 mL). The organic layer was removed and the aqueous layer extracted with DCM (50 mL). The combined organic fractions were dried over sodium sulfate, filtered and the solvent removed in vacuo to afford a yellow oil. The oil was dissolved in 3 mL of DMF and purified by Isotec CombiFlash chromatography using a RediSepR_{f} Gold 150 g HP C18 (aq) RP column eluting with a gradient from 1:9 to 4:1 ACN:water (NO added TFA modifier). The desired fractions were collected and the ACN removed in vacuo. The aqueous layer was poured into a separatory funnel and extracted with DCM (40 mL×3). The combined organic fractions were dried over sodium sulfate, filtered and the solvent removed in vacuo to afford 950 mg (72% yield for the two steps) of Compound S33-2 as a yellow oil which slowly solidifies. MS (ESI) m/z [M+H]⁺ calcd. for C₁₅H₁₇N₄O: 269.1. Found: 269.1.

### 3. Synthesis of Compound S33

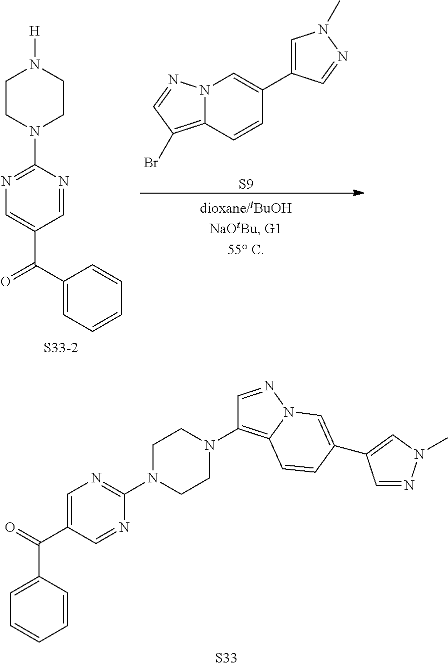

3-Bromo-6-(1-methyl-1H-pyrazol-4-yl)pyrazolo[1,5-a]pyridine (Compound S9, 0.234 g, 0.84 mmol), phenyl(2-piperazin-1-ylpyrimidin-5-yl)methanone (Compound S33-2, 0.26 g, 0.97 mmol), tert-butyl alcohol (2.3 mL) and 1,4-dioxane (1.1 mL) were all taken up in a 100 mL round bottom flask which had been flushed under argon. The slurry was warmed to 50° C., and sparged with Ar for 10 minutes. Sodium tert-butoxide (122 mg, 1.27 mmol) and tBuXPhos Pd G1 (116 mg, 0.17 mmol) were then added quickly and the now darkened slurry stirred for 5 minutes with continued argon sparging. The reaction was capped and stirred at 55° C. for 2 hours. The reaction mixture was cooled to rt and the resultant solids were collected on a tared Buchner funnel washing with minimal THF. The solids were dried at rt and afforded 160 mg (41% yield) of Compound S33 as a brown solid. MS (ESI) m/z [M+H]⁺ calcd. for C₂₆H₂₅N₈O: 465.2. Found: 465.2.

### Synthesis of Compound S34 (2-azetidin-3-yl-5-benzylpyrimidine)

### 1. Synthesis of Intermediate Compound S34-1

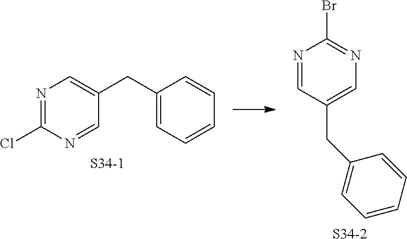 [SMILES: "S34-1" ; Clc1ncc(Cc2ccccc2)cn1 ; Brc1ncc(Cc2ccccc2)cn1 ; "S34-2"]

To a solution of 5-benzyl-2-chloropyrimidine (Compound S34-1, 1.00 g, 4.89 mmol) in propanenitrile (5.0 mL) was added bromotrimethylsilane (1.3 mL, 9.6 mmol). The reaction was warmed to reflux. After 5 h at reflux the reaction was recharged with bromotrimethylsilane (1.3 mL, 9.8 mmol) and propanenitrile (5 mL), and was allowed to continue to reflux overnight. The reaction was cooled to room temperature and poured onto a mixture of ice and 2 M NaOH (10 mL). The quenched reaction was extracted with ether (3×10 mL). The combined ether layers were washed with water (1×10 mL) then brine (1×10 mL), dried (Na₂SO₄), filtered, and concentrated to afford 1.16 g (95%) of 5-benzyl-2-bromopyrimidine (Compound S34-2) as a tan waxy solid: MS (ESI) m/z [M+H]⁺ calcd. for C₁₁H₁₀BrN₂, 249.0; found, 248.9.

### 2. Synthesis of Intermediate Compound S34-4

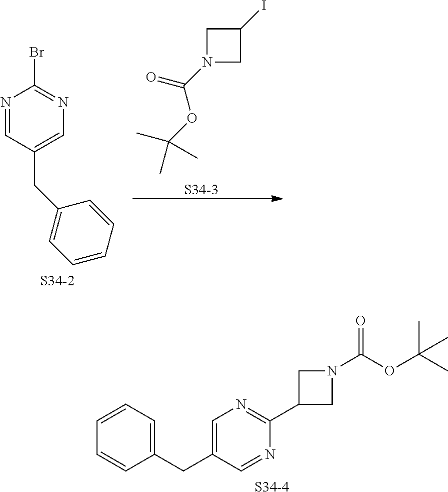 [SMILES: "S34-2" ; "S34-3" ; "S34-4" ; Brc1ncc(Cc2ccccc2)cn1 ; CC(C)(C)OC(=O)N1CC(c2ncc(Cc3ccccc3)cn2)C1 ; CC(C)(C)OC(=O)N1CC(I)C1]

A vial of zinc (0.196 g, 3.00 mmol) and dry THF (2.6 mL) was sparged with argon for 5 min. To the sparged suspension was added 1,2-dibromoethane (20 ul, 0.20 mmol) and the reaction was placed in an oil bath at 60° C. After 15 min the reaction was cooled to room temperature. To the reaction was added chlorotrimethylsilane (20 ul, 0.10 mmol). After stirring 50 min., a solution of tert-butyl 3-iodoazetidine-1-carboxylate (Compound S34-3, 0.50 g, 1.8 mmol) in THF (1.3 mL) was added dropwise to the reaction. After 1.5 h, tetrakis(triphenylphosphine) palladium (0) (20 mg, 0.02 mmol) was added to the reaction. The reaction was sparged with argon for 5 min. Added a solution of 5-benzyl-2-bromopyrimidine (Compound S34-2, 0.530 g, 2.13 mmol) dissolved in THF (3 mL). The reaction was warmed to reflux. After 3h, additional THF (2 mL) was added and the reaction was allowed to reflux overnight. After 16 h the reaction was cooled to room temperature and filtered through a pad of Celite washing with THF. The filtrate was concentrated, and the residue was partitioned between EtOAc (50 mL), sat. aq. NaHCO₃ (10 mL) and water (10 mL). The aqueous layer was diluted with brine (20 mL) and washed with EtOAc (2×20 mL). The combined EtOAc layers were dried (Na₂SO₄) and concentrated. The residue was purified by ISCO chromatography (40 g normal phase cartridge) eluting with a gradient of 0-100% ethyl acetate in hexanes to afford 242 mg (42%) of tert-butyl 3-(5-benzylpyrimidin-2-yl) azetidine-1-carboxylate (Compound S34-4) as a dark oil: MS (ESI) m/z [M+H]⁺ calcd. for C₁₉H₂₄N₃O₂, 326.2; found, 326.2.

### 3. Synthesis of Compound S34

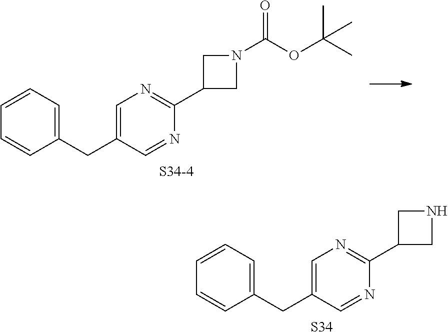 [SMILES: "S34-4" ; CC(C)(C)OC(=O)N1CC(c2ncc(Cc3ccccc3)cn2)C1 ; c1ccc(Cc2cnc(C3CNC3)nc2)cc1 ; "S34"]

To a solution of tert-butyl 3-(5-benzylpyrimidin-2-yl) azetidine-1-carboxylate (Compound S34-4, 0.225 g, 0.69 mmol) in MeOH (10 mL) was added 0.80 g of freshly washed and dried Dowex 50WX4-400 ion exchange resin. The suspension was stirred gently at 50° C. for 4 h then cooled to room temperature. The suspension was filtered, and the resin washed with MeOH (5 mL), DCM (5 mL) and MeOH (5 mL). The resin was then washed with 3.5 M NH₃ in MeOH (6×5 mL). The methanolic ammonia filtrate was concentrated to afford 153 mg (98%) of 2-azetidin-3-yl-5-benzylpyrimidine (Compound S34) as a colorless oil. MS (ESI) m/z [M+H]⁺ calcd. for C₁₄H₁₆N₃, 226.1; found, 226.1.

### Synthesis of Compound S35 ((4-Fluorophenyl)(2-{4-[6-(1-methyl-1H-pyrazol-4-yl)pyrazolo[1,5-a]pyrimidin-3-yl]piperazin-1-yl}pyrimidin-5-yl)methanone)

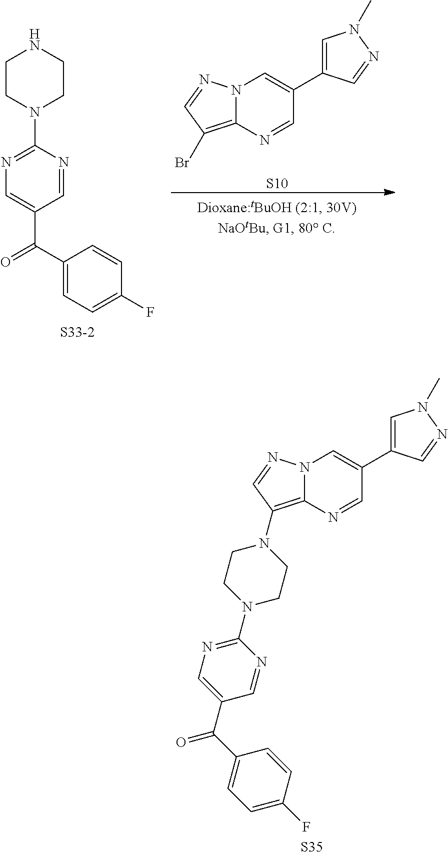 [SMILES: O=C(c1ccc(F)cc1)c1cnc(N2CCNCC2)nc1 ; "S10" ; Cn1cc(-c2cnc3c(Br)cnn3c2)cn1 ; "S33-2" ; Cn1cc(-c2cnc3c(N4CCN(c5ncc(C(=O)c6ccc(F)cc6)cn5)CC4)cnn3c2)cn1 ; "S35"]

3-Bromo-6-(1-methyl-1H-pyrazol-4-yl)pyrazolo[1,5-a]pyrimidine (Compound S10, 0.35 g, 1.24 mmol), (4-fluorophenyl)(2-piperazin-1-ylpyrimidin-5-yl)methanone (See Example 28, Step 5; 0.40 g, 1.40 mmol), sodium tert-butoxide (179 mg, 1.86 mmol), tert-butyl alcohol (2.3 mL) and 1,4-dioxane (4.6 mL) were all charged into a 100 mL round bottom flask which had been flushed under argon. The slurry was sparged with Ar for 10 minutes. The mixture was warmed to 50° C. at which point tBuXPhos Pd G1 (171 mg, 0.25 mmol) was added, the reaction mixture sparged with Ar for 5 minutes and the reaction mixture heated at 80° C. under Ar for 2 hours. The reaction mixture was cooled to rt affording a thick slurry. The solvent was removed in vacuo to afford a dark yellow solid. The material was taken up in 10 mL DMF and 4 mL DMSO and the solids were collected by filtration washing with water. The solids were dried in a vacuum oven at 50° C. overnight to provide 90 mg (15%) of the desired product (Compound S35) as a tan solid. Used as is for the next step (e.g., Example 77). MS (ESI) m/z [M+H]⁺ calcd. for C₂₅H₂₂FN₉O: 483.2. Found: 484.2.

### Synthesis of Compound S36 (3-[6-(1-methyl-1H-pyrazol-4-yl)pyrazolo[1,5-a]pyridin-3-yl]pyrrolidinium trifluoroacetate)

### 1. Synthesis of Intermediate Compound S36-1

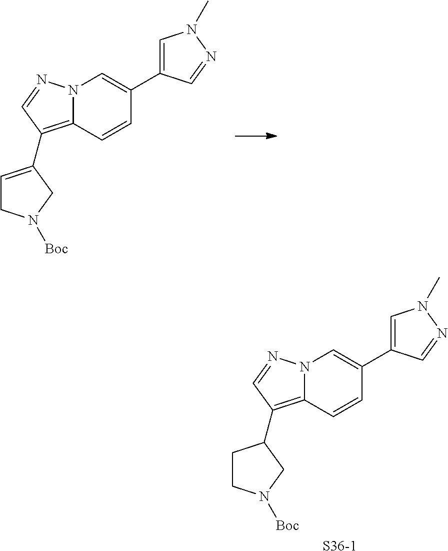 [SMILES: Cn1cc(-c2ccc3c(C4=CCN(C(=O)OC(C)(C)C)C4)cnn3c2)cn1 ; Cn1cc(-c2ccc3c(C4CCN(C(=O)OC(C)(C)C)C4)cnn3c2)cn1 ; "S36-1"]

A solution of tert-butyl 3-[6-(1-methyl-1H-pyrazol-4-yl)pyrazolo[1,5-a]pyridine-3-yl]-2,5-dihydro-1H-pyrrole-1-carboxylate (See Example 54, Step 1; 97 mg, 0.26 mmol) in DMF (9.7 mL) was treated with 10% palladium on carbon (194 mg; 50% water wet). The reaction flask was evacuated and filled with hydrogen gas three times and the reaction was stirred under an atmosphere of hydrogen. After 22 h the starting material was consumed as indicated by HPLC. The reaction mixture was filtered through a pad of celite, washing the pad with an additional 20 mL EtOAc. The filtrate was washed with 15 mL H₂O, dried over MgSO₄, filtered and concentrated to yield 95 mg (97%) of crude tert-butyl 3-[6-(1-methyl-1H-pyrazol-4-yl)pyrazolo[1,5-a]pyridin-3-yl]pyrrolidine-1-carboxylate (Compound S36-1) as a glass that was of suitable purity for use in the next step: ¹H NMR (400 MHZ, CDCl₃) δ (ppm) 8.54 (s, 1H) 7.79 (s, 1H) 7.74 (s, 1H) 7.62 (s, 1H) 7.49 (d, J=9.17 Hz, 1H) 7.22 (d, J=4.89 Hz, 1H) 3.97 (s, 3H) 3.78-3.96 (m, 1H) 3.52-3.73 (m, 2H) 3.27-3.52 (m, 2H) 2.33 (br. s., 1H) 1.98-2.15 (m, 1H) 1.49 (br. s., 9H); MS (ESI) m/z [M+H]⁺ calcd. for C₂₀H₂₆N₅O₂: 368.2; found, 368.3; HPLC purity: 210 nm: 96.1%; 254 nm: 97.4%.

### 2. Synthesis of Compound S36

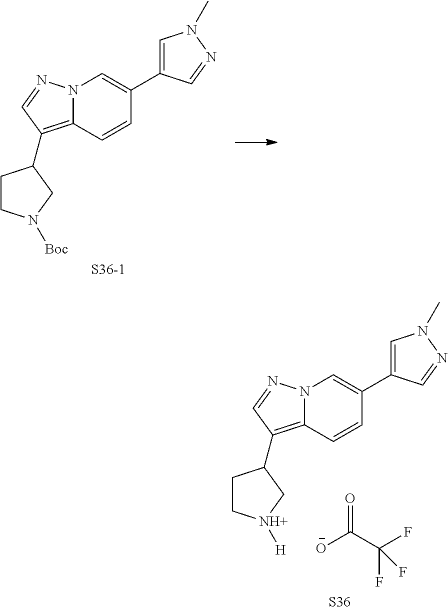 [SMILES: Cn1cc(-c2ccc3c(C4CCN(C(=O)OC(C)(C)C)C4)cnn3c2)cn1 ; "S36-1" ; Cn1cc(-c2ccc3c(C4CC[NH2+]C4)cnn3c2)cn1 ; O=C([O-])C(F)(F)F ; "S36"]

A solution of tert-butyl 3-[6-(1-methyl-1H-pyrazol-4-yl)pyrazolo[1,5-a]pyridin-3-yl]pyrrolidine-1-carboxylate (Compound S36-1, 95 mg, 0.26 mmol) in CH₂Cl₂ (5.0 mL) was cooled at 0° C., and treated dropwise with TFA (0.50 mL, 6.0 mmol). The solution was stirred for 30 min at 0° C. following by warming to RT. After 1.5 h, HPLC indicated the starting material had been consumed. The reaction mixture was concentrated to a grey residue that was taken up in 4 mL CH₂Cl₂ and concentrated. The process was repeated once and the resultant residue placed under high vac to yield crude 3-[6-(1-methyl-1H-pyrazol-4-yl)pyrazolo[1,5-a]pyridin-3-yl]pyrrolidinium trifluoroacetate (Compound S36) that was determined to be of suitable purity for use in the next step (assumed a 100% yield): ¹H NMR (400 MHZ, CD₃OD) δ (ppm) 8.72 (s, 1H) 8.05 (s, 1H) 7.94 (s, 1H) 7.89 (s, 1H) 7.67-7.73 (m, 1H) 7.49 (dd, J=9.23, 1.41 Hz, 1H) 3.95 (s, 3H) 3.72-3.89 (m, 2H) 3.59 (ddd, J=11.86, 8.31, 3.79 Hz, 1H) 3.39-3.50 (m, 1H) 3.24-3.36 (m, 2H) 2.48-2.64 (m, 1H) 2.13-2.32 (m, 1H); MS (ESI) m/z [M+H]⁺ calcd. for C₁₅H18Ns: 268.2; found, 268.1; HPLC purity: 210 nm: 100%; 254 nm: 100%.

### Synthesis of Compound S37 (4-nitrophenyl isopropyl(methyl)carbamate)

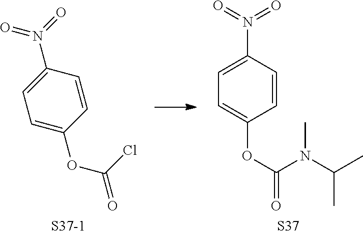 [SMILES: O=C(Cl)Oc1ccc([N+](=O)[O-])cc1 ; CC(C)N(C)C(=O)Oc1ccc([N+](=O)[O-])cc1 ; "S37-1" ; "S37"]

To a cooled solution of N-isopropylmethylamine (45 mg, 0.62 mmol) in DCM (6.4 mL) at 3° C. was added pyridine (211 μL, 2.60 mmol) and after stirring 10 minutes 4-nitrophenyl chlorocarbonate (Compound S37-1, 0.250 g, 1.24 mmol). Cooling was stopped and the solution stirred at room temperature overnight. The mixture was concentrated under vacuum and the residue was concentrated from heptane to remove pyridine. The residue was purified by Biotage chromatography (20 g) cartridge eluting with a gradient of DCM in hexanes to afford 0.130 g (74%) of 4-nitrophenyl isopropyl(methyl)carbamate (Compound S37) as an oil: 1H NMR (400 MHZ, CDCl₃) ¹H δ (ppm) 8.29 (d, J=9.17 Hz, 2H) 7.40 (d, J=9.17 Hz, 2H) 4.74 (t, J=6.17 Hz, 1H) 1.63-1.85 (m, 4H) 1.00 (t, J=7.46 Hz, 6H).

### Synthesis of Compound S38 (5-benzyl-2-chloropyrimidine-D₇)

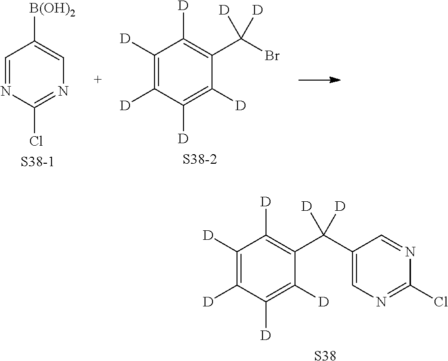 [SMILES: OB(O)c1cnc(Cl)nc1 ; [2H]c1c([2H])c([2H])c(C([2H])([2H])Br)c([2H])c1[2H] ; "S38-2" ; "S38-1" ; [2H]c1c([2H])c([2H])c(C([2H])([2H])c2cnc(Cl)nc2)c([2H])c1[2H] ; "S38"]

A mixture of 2-(chloropyrimidine-5-yl) boronic acid (Compound S38-1, 1.20 g, 7.58 mmol), sodium carbonate (2.41 g, 22.7 mmol) and bis(triphenylphosphine) palladium (II) chloride (0.266 g, 0.379 mmol) in 1,4-dioxane (5.9 mL) and deuterium oxide (2.5 mL) was degassed by sparging with argon gas for 10 min. The solution was treated with bromomethylbenzene-D7 (Compound S38-2, 1.48 g, 8.34 mmol) and the reaction mixture was heated at 100° C. After 1.25 h the reaction was determined to be complete and cooled to RT. The reaction mixture was diluted with 40 mL EtOAc and 50 mL H₂O, extracted and the aqueous phase was washed with 20 mL EtOAc. The combined organic phases was washed with 25 mL portions of H₂O and brine and dried over MgSO₄. The solution was filtered through a pad of magnesol and concentrated to an orange oil that was purified by flash chromatography (125 g silica gel, 10-20% MTBE/hex) to yield 0.83 g (52%) of 5-benzyl-2-chloropyrimidine-D7 (Compound S38) as a tan solid: ¹H NMR (400 MHZ, CDCl₃) δ (ppm) 8.47 (s, 2H); MS (ESI) m/z [M+H]⁺ calcd. for C₁₁H₂ClD₇N₂: 212.1; found, 212.1/214.0; HPLC purity: 210 nm: 97.7%; 254 nm: 100%.

### Synthesis of 6-(1-(2-methoxyethyl)-1H-pyrazol-4-yl)pyrimidin-4-amine (Compound S39)

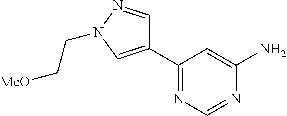 [SMILES: COCCn1cc(-c2cc(N)ncn2)cn1]

To a mixture of 6-chloropyrimidin-4-amine (6.0 g, 46.3 mmol) and 1-(2-methoxyethyl)-4-(4,4,5,5-tetramethyl-1,3,2-dioxaborolan-2-yl)-1H-pyrazole (15.2 g, 60.1 mmol) in dioxane/water=5/1 (120 mL) was added Na₂CO₃ (9.8 g, 92.6 mmol) and Pd(dppf)Cl₂ (1.2 g) under nitrogen. The mixture was heated to 100° C., and stirred for 6 h under nitrogen. The mixture was cooled to 25° C., then poured into cold water (400 mL) and extracted with 10% MeOH/DCM (10×80 mL). The combined organic phase was washed with brine, dried over anhydrous Na₂SO₄, and filtered. The filtrate was concentrated under reduced pressure then the residue was purified by silica gel chromatography to afford 3.0 g (28%) of 6-(1-(2-methoxyethyl)-1H-pyrazol-4-yl)pyrimidin-4-amine (Compound S39) as a yellow solid. ¹H NMR (400 MHZ, DMSO-d₆) δ (ppm) 8.30 (s, 1H), 8.22 (s, 1H), 7.91 (s, 1H), 6.73 (s, 2H), 6.56 (d, J=0.8 Hz, 1H), 4.30 (t, J=5.2 Hz, 2H), 3.70 (t, J=5.2 Hz, 2H), 3.24 (s, 3H). MS (ESI) m/z [M+H]⁺ calcd. for C₁₀H₁₃N₅O, 220.1; found, 219.8.

Using the procedures described for Compound S39 and reagents, starting materials, and conditions known to those skilled in the art, the following compounds were prepared:

| Compound | Name | Characterization |
|---|---|---|
| Compound S40 | 6-(pyridin-3-yl)pyrimidin-4- | ¹H NMR (400 MHz, DMSO- |
| | amine | d₆) δ (ppm) 9.14 (d, J = 2.0 |
| | | Hz, 1H), 8.66 (s, 1H), 8.48 (s, |
| | | 1H), 8.30-8.33 (m, 1H), 7.52 |
| | | (d, J = 8.0 Hz, 1H), 7.03 (s, |
| | | 2H), 6.94 (d, J = 0.8 Hz, 1H). |
| | | MS (ESI) m/z [M + H]⁺ calcd. |
| | | for C₉H₈N₄, 173.1; found, |
| | | 172.7. |
| Compound S43 | 4-(1-(2-methoxyethyl)-1H- | ¹H NMR (400 MHz, CDCl₃) |
| | pyrazol-4-yl)pyrimidin-2- | δ (ppm) 8.22 (d, J = 5.2 Hz, |
| | amine | 1H), 8.05 (s, 1H), 7.99 (s, |
| | | 1H), 6.76 (d, J = 5.2 Hz, 1H), |
| | | 5.17 (s, 2H), 4.33 (t, J = 5.2 |
| | | Hz, 2H), 3.77 (t, J = 5.2 Hz, |
| | | 2H), 3.34 (s, 3H). MS (ESI) |
| | | m/z [M + H]⁺ calcd. for |
| | | C₁₀H₁₃N₅O, 220.1; found, |
| | | 219.8. |
| Compound S44 | 4-(pyridin-3-yl)pyrimidin-2- | ¹H NMR (400 MHz, DMSO- |
| | amine | d₆) δ (ppm) 9.25 (d, J = 2.0 |
| | | Hz, 1H), 8.69 (d, J = 4.8 Hz, |
| | | 1H), 8.37~8.41 (m, 2H), |
| | | 7.49~7.54 (m, 1H), 7.22 (d, J = |
| | | 5.2 Hz, 1H), 6.81 (s, 2H). |
| | | MS (ESI) m/z [M + H]⁺ calcd. |
| | | for C₉H₈N₄, 173.1; found, |
| | | 173.2. |

### Synthesis of 6-(tetrahydrofuran-3-yl)pyrimidin-4-amine (Compound S41)

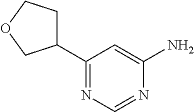 [SMILES: Nc1cc(C2CCOC2)ncn1]

### Step 1. Synthesis of 6-(2,5-dihydrofuran-3-yl)pyrimidin-4-amine

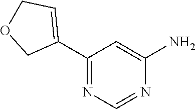 [SMILES: Nc1cc(C2=CCOC2)ncn1]

To a mixture of 6-chloropyrimidin-4-amine (4.0 g, 30.9 mmol) and 2-(2,5-dihydrofuran-3-yl)-4,4,5,5-tetramethyl-1,3,2-dioxaborolane (7.9 g, 40.2 mmol) in dioxane/water=5/1 (120 mL) was added Na₂CO₃ (6.6 g, 61.8 mmol) and Pd(dppf)Cl₂ (0.5 g) under nitrogen. The mixture was stirred at 100° C. for 6 h under nitrogen. The mixture was concentrated under reduced pressure then the residue was purified by silica gel chromatography (DCM:MeOH=50:1˜20:1) to afford 4.0 g (63%) of 6-(2,5-dihydrofuran-3-yl)pyrimidin-4-amine as a yellow solid. ¹H NMR (400 MHZ, DMSO-d₆) δ (ppm) 8.31 (s, 1H), 6.87 (s, 2H), 6.74 (t, J=2.0 Hz, 1H), 6.36 (s, 1H), 4.84-4.82 (m, 2H), 4.75-4.72 (m, 2H). MS (ESI) m/z [M+H]⁺ calcd. for C₈H₉N₃O, 164.1; found, 163.7.

### Step 2. Synthesis of 6-(tetrahydrofuran-3-yl)pyrimidin-4-amine (Compound S41)

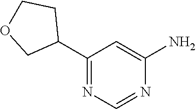 [SMILES: Nc1cc(C2CCOC2)ncn1]

To a solution of 6-(2,5-dihydrofuran-3-yl)pyrimidin-4-amine (4.0 g, 24.5 mmol, as prepared in the previous step) in MeOH (80 mL) was added Pd/C (50% H₂O, 1.0 g). The mixture was stirred at 25° C. under H₂ (20 psi) for 16 h. The mixture was filtered and the filtrate was concentrated in vacuum to give the crude solid. The crude solid was purified by silica gel chromatography (DCM:MeOH=50:1˜30:1) to afford 3.4 g (80%) of 6-(tetrahydrofuran-3-yl)pyrimidin-4-amine (Compound S41) as an off-white solid. ¹H NMR (400 MHZ, DMSO-d₆) δ (ppm) 8.27 (s, 1H), 6.76 (s, 2H), 6.30 (s, 1H), 3.96 (t, J=8.0 Hz, 1H), 3.85-3.83 (m, 1H), 3.78-3.73 (m, 1H), 3.63 (t, J=8.0 Hz, 1H), 3.28-3.20 (m, 1H), 2.22-2.13 (m, 1H), 2.05-1.96 (m, 1H). MS (ESI) m/z [M+H]⁺ calcd. for C₈H₁₁N₃O, 166.1; found, 165.7.

Using the procedures described for Compound S41 and reagents, starting materials, and conditions known to those skilled in the art, the following compounds were prepared:

| Compound | Name | Characterization |
|---|---|---|
| Compound S45 | 4-(tetrahydrofuran-3- | ¹H NMR (400 MHz, DMSO- |
| | yl)pyrimidin-2-amine | d₆) δ (ppm) 8.12 (d, J = 5.2 |
| | | Hz, 1H), 6.52-6.49 (m, 3H), |
| | | 3.97 (t, J = 8.0 Hz, 1H), 3.87- |
| | | 3.83 (m, 1H), 3.78-3.66 |
| | | (m, 2H), 3.31-3.23 (m, 1H), |
| | | 2.25-2.14 (m, 1H), 2.05- |
| | | 2.00 (m, 1H). MS (ESI) m/z |
| | | [M + H]⁺ calcd. for |
| | | C₈H₁₁N₃O, 166.1; found, |
| | | 165.7. |

### Synthesis of 6-(4-methyl-1H-imidazol-1-yl)pyrimidin-4-amine (Compound S42)

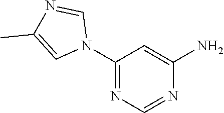

To a mixture of 6-chloropyrimidin-4-amine (8.0 g, 61.8 mmol) and 4-methyl-1H-imidazole (6.1 g, 74.1 mmol) in DMF (80 mL) was added Cs₂CO₃ (24.2 g, 74.1 mmol) under nitrogen. The mixture was heated to 120° C., and stirred under nitrogen for 12 h. The mixture was cooled, poured into cold water, and extracted with DCM/MeOH=10/1 ten times. The organic phase was washed with brine, dried over Na₂SO₄, and concentrated under reduced pressure then the residue was purified by silica gel chromatography to afford 2.4 g (20%) of 6-(4-methyl-1H-imidazol-1-yl)pyrimidin-4-amine (Compound S42) as a yellow solid. ¹H NMR (400 MHZ, DMSO-d₆) δ (ppm) 8.30-8.33 (m, 2H), 7.50 (s, 1H), 7.15 (s, 2H), 6.49 (d, J=0.8 Hz, 1H), 2.12-2.16 (m, 3H). MS (ESI) m/z [M+H]⁺ calcd. for C₈H₉N₅, 176.1; found, 175.7.

Using the procedures described for Compound S42 and reagents, starting materials, and conditions known to those skilled in the art, the following compounds were prepared:

| Compound | Name | Characterization |
|---|---|---|
| Compound S46 | 4-(4-methyl-1H-imidazol-1- | ¹H NMR (400 MHz, DMSO- |
| | yl)pyrimidin-2-amine | d₆) δ (ppm) 8.40 (d, J = 1.2 |
| | | Hz, 1H), 8.31 (d, J = 5.6 Hz, |
| | | 1H), 7.58~7.59 (m, 1H), |
| | | 6.88~6.91 (m, 3H), 2.16 (d, J = |
| | | 0.8 Hz, 3H). MS (ESI) m/z |
| | | [M + H]⁺ calcd. for C₈H₉N₅, |
| | | 176.1; found, 175.8. |

### Synthesis of di-tert-butyl 4,4′-(1,2-bis(1H-benzo[d][1,2,3]triazol-1-yl)ethane-1,2-diyl)bis(piperazine-1-carboxylate) (Compound S47)

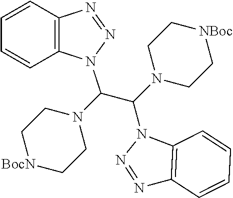

A solution of 1H-1,2,3-benzotriazole (19.2 g, 161.1 mmol) and tert-butyl piperazine-1-carboxylate (30.0 g, 161.1 mmol) in EtOH (300 mL) was stirred under nitrogen at 25° C. for 20 min. Then glyoxal (40 wt. % in H₂O, 4.7 g, 80.5 mmol) was added to the mixture at 25° C. The reaction mixture was stirred at 30° C. for 16 h. The reaction mixture was concentrated under reduced pressure then PE was added. The mixture was stirred for 30 min and filtered, the filter cake was dried to afford 42.0 g (32%) of di-tert-butyl 4,4′-(1,2-bis(1H-benzo[d][1,2,3]triazol-1-yl)ethane-1,2-diyl)bis(piperazine-1-carboxylate (Compound S47) as a white solid.

Using the procedures described for Compound S47 and reagents, starting materials, and conditions known to those skilled in the art, the following compounds were prepared:

| Compound | Name |
|---|---|
| Compound S48 | di-tert-butyl 4,4′-(1,2-bis(1H-benzo[d][1,2,3 ]triazol-1- |
| | yl)ethane-1,2-diyl)bis(1,4-diazepane-1-carboxylate) |

### Synthesis of di-tert-butyl (((1,2-bis(1H-benzo[d][1,2,3]triazol-1-yl)ethane-1,2-diyl)bis(methylazanediyl))bis(ethane-2,1-diyl))bis(methylcarbamate) (Compound S49)

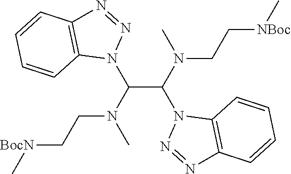

A solution of 1H-benzo[d][1,2,3]triazole (6.3 g, 52.9 mmol) and tert-butyl methyl(2-(methylamino)ethyl)carbamate (10.0 g, 52.9 mmol) in EtOH (300 mL) was stirred under nitrogen at 25° C. for 20 min then glyoxal (40 wt. % in H₂O, 3.8 g, 26.2 mmol) was added to the mixture at 25° C. The reaction mixture was stirred at rt for 16 h. The reaction mixture was concentrated under reduced pressure then the residue was washed with PE to afford 11.0 g (59%) di-tert-butyl (((1,2-bis(1H-benzo[d][1,2,3]triazol-1-yl)ethane-1,2-diyl)bis(methylazanediyl))bis(ethane-2,1-diyl))bis(methylcarbamate (Compound S49) as a white solid.

Using the procedures described for Compound S49 and reagents, starting materials, and conditions known to those skilled in the art, the following compounds were prepared:

| Compound | Name |
|---|---|
| Compound S50 | di-tert-butyl 1,1′-(1,2-bis(1H-benzo[d][1,2,3]triazol-1- |
| | yl)ethane-1,2-diyl)bis(pyrrolidine-3-carboxylate) |

### Synthesis of (R)-4-nitrophenyl (1-phenylethyl)carbonate (Compound S51)

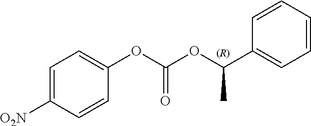

To a solution of 4-nitrophenyl chloroformate (4.0 g, 19.8 mmol) and (IR)-1-phenylethanol (1.21 g, 9.9 mmol) in DCM (50 mL) was added pyridine (1.57 g, 19.8 mmol) at 25° C. The reaction mixture was stirred at 25° C. for 3 h. The mixture was concentrated under reduced pressure then the residue was purified by silica gel chromatography to afford 3.7 g (65%) of (R)-4-nitrophenyl (1-phenylethyl)carbonate (Compound S51) as a yellow oil. ¹H NMR (400 MHz, CDCl₃) δ (ppm) 8.21-8.28 (m, 2H), 7.25-7.41 (m, 7H), 5.84 (q, J=6.4 Hz, 1H), 1.70 (d, J=6.4 Hz, 3H).

### Synthesis of 7-(1-methyl-1H-pyrazol-4-yl)-3-(piperazin-1-yl)imidazo[1,2-c]pyrimidine hydrochloride Salt (Compound S52)

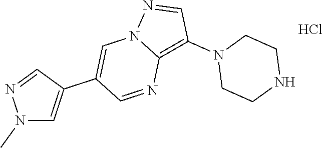 [SMILES: Cn1cc(-c2cnc3c(N4CCNCC4)cnn3c2)cn1 ; Cl]

To a solution of tert-butyl 4-(7-(1-methyl-1H-pyrazol-4-yl)imidazo[1,2-c]pyrimidin-3-yl)piperazine-1-carboxylate (Compound 83) (400.0 mg, 1.04 mmol) in DCM (3 mL) was added 4M HCl in dioxane (10 mL) at 25° C. The mixture was stirred at 25° C. for 2 h. The mixture was concentrated in vacuum to give a yellow solid. The crude solid was washed with MTBE and filtered. The filter cake was dried to afford 360.0 mg of 7-(1-methyl-1H-pyrazol-4-yl)-3-(piperazin-1-yl)imidazo[1,2-c]pyrimidine hydrochloride salt (Compound S52) as a yellow solid. MS (ESI) m/z [M+H]⁺ calcd. for C₁₄H₁₇N₇, 284.2; Found, 284.1.

Using the procedures described for Compound S52 and reagents, starting materials, and conditions known to those skilled in the art, the following compounds were prepared:

| Compound | Name | Characterization |
|---|---|---|
| Compound S53 | 3-(1,4-diazepan-1-yl)-7-(1- | MS (ESI) m/z [M + H]⁺ calcd. |
| | methyl-1H-pyrazol-4- | for C₁₅H₁₉N₇, 298.2; Found, |
| | yl)imidazo[1,2-c]pyrimidine | 298.1. |
| | hydrochloride salt | |
| Compound S54 | 7-(1-methyl-1H-pyrazol-4- | ¹H NMR (400 MHz, DMSO- |
| | yl)-3-(piperazin-1- | d₆) δ (ppm) 9.67 (br s, 2H), |
| | yl)imidazo[1,2-a]pyrimidine | 9.15 (d, J = 7.2 Hz, 1H), 8.77 |
| | hydrochloride salt | (s, 1H), 8.35 (s, 1H), 8.04 (s, |
| | | 1H), 7.92 (d, J = 7.2 Hz, 1H), |
| | | 3.97 (s, 1H), 3.29 (s, 8H). MS |
| | | (ESI) m/z [M + H]⁺ calcd. for |
| | | C₁₄H₁₇N₇, 284.2; Found, |
| | | 283.9. |
| Compound S55 | 3-(1,4-diazepan-1-yl)-7-(1- | MS (ESI) m/z [M + H]⁺ calcd. |
| | methyl-1H-pyrazol-4- | for C₁₅H₁₉N₇, 298.2; Found, |
| | yl)imidazo[1,2-a]pyrimidine | 298.1. |
| | hydrochloride salt | |

### Synthesis of 6-(1-methyl-1H-pyrazol-4-yl)pyrazolo[1,5-a]pyridin-3-amine (Compound S56)

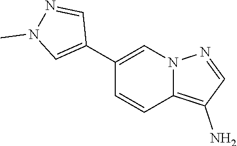 [SMILES: Cn1cc(-c2ccc3c(N)cnn3c2)cn1]

### Step 1: Synthesis of 6-(1-methyl-1H-pyrazol-4-yl)-3-nitropyrazolo[1,5-a]pyridine

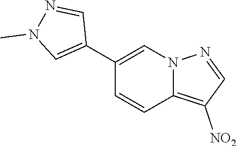 [SMILES: Cn1cc(-c2ccc3c([N+](=O)[O-])cnn3c2)cn1]

To a solution of 6-(1-methyl-1H-pyrazol-4-yl)pyrazolo[1,5-a]pyridine (4.0 g, 5.05 mmol, as prepared in Compound S9, Step 1) dissolved in H₂SO₄ (10 mL) was added KNO₃ (2.24 g, 5.55 mmol). The reaction mixture was stirred for 1 h at rt under nitrogen. The mixture was cooled to 0° C., and diluted with water (300 mL). The mixture was neutralized to pH 8 with sat. aq. Na₂CO₃. The precipitated solids were collected by filtration and washed with water (3×300 mL). After filtration, the precipitated solids were dried under reduced pressure to afford 3.5 g (71%) of 6-(1-methyl-1H-pyrazol-4-yl)-3-nitropyrazolo[1,5-a]pyridine as a yellow solid. ¹H NMR (400 MHZ, DMSO-d₆) δ (ppm) 9.34 (s, 1H), 8.90 (s, 1H), 8.38 (s, 1H), 8.24 (d, J=9.1 Hz, 1H), 8.15 (d, J=9.3 Hz, 1H), 8.11 (s, 1H), 3.90 (s, 3H); MS (ESI) m/z [M+H]⁺ calcd. for C₁₁H₉N₅O₂, 244.1; found, 244.1.

### Step 2: Synthesis of 6-(1-methyl-1H-pyrazol-4-yl)pyrazolo[1,5-a]pyridin-3-amine (Compound S56)

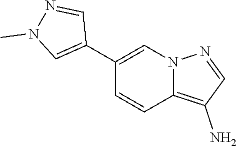 [SMILES: Cn1cc(-c2ccc3c(N)cnn3c2)cn1]

To a solution of 6-(1-methyl-1H-pyrazol-4-yl)-3-nitropyrazolo[1,5-a]pyridine (3.0 g, 12.33 mmol, as prepared in the previous step) dissolved in HBr (aq.) (50 mL) was added SnCl₂·2H₂O (8.42 g, 37.00 mmol). The resulting solution was stirred for 4 h at 90° C. under nitrogen. The mixture was cooled to rt then basified to pH 9 with NaOH (aq.). The resulting mixture was extracted with EtOAc (3×500 mL). The combined organic layers were washed with brine (3×100 mL), dried over anhydrous Na₂SO₄, and filtered. The filtrate was concentrated under reduced pressure to afford 2.5 g (90%) of 6-(1-methyl-1H-pyrazol-4-yl)pyrazolo[1,5-a]pyridin-3-amine (Compound S56) as a brown yellow solid. ¹H NMR (400 MHz, DMSO-d₆) δ (ppm) 8.63 (s, 1H), 8.15 (s, 1H), 7.92 (s, 1H), 7.59 (d, J=9.2 Hz, 1H), 7.44 (s, 1H), 7.11 (d, J=9.2 Hz, 1H), 4.33 (s, 2H), 3.86 (s, 3H); MS (ESI) m/z [M+H]⁺ calcd. for C₁₁H₁₁N₅, 214.1; found, 214.1.

### Synthesis of 6-(1-methyl-1H-pyrazol-4-yl)-3-(4,4,5,5-tetramethyl-1,3,2-dioxaborolan-2-yl)pyrazolo[1,5-a]pyridine (Compound S57)

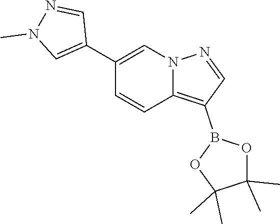

### Step 1. Preparation of 3-iodo-6-(1-methyl-1H-pyrazol-4-yl)pyrazolo[1,5-a]pyridine

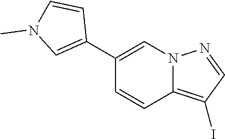 [SMILES: Cn1ccc(-c2ccc3c(I)cnn3c2)c1]

To a solution of 6-(1-methyl-1H-pyrazol-4-yl)pyrazolo[1,5-a]pyridine (7.0 g, 35.3 mmol, as prepared in Compound S9, Step 1) in DMF (70 mL) was added NIS (9.53 g, 42.4 mmol). The resulting mixture was stirred for 1 h at rt. The reaction mixture was poured into a stirred mixture of 2.8 M Na₂S203 solution (64 mL) and 1.14 M NaHCO₃ solution (144 mL). The mixture was stirred vigorously for 1 h resulting in an off-white precipitate. The solids were collected by filtration, washed with H₂O (2×10 mL), and dried under high vacuum to afford 10 g (79%) of 3-iodo-6-(1-methyl-1H-pyrazol-4-yl)pyrazolo[1,5-a]pyridine as a white solid. ¹H NMR (400 MHZ, DMSO-d₆) (ppm) δ 9.06 (s, 1H), 8.28 (s, 1H), 8.08 (s, 1H), 8.02 (d, J=0.8 Hz, 1H), 7.60 (d, J=9.2 Hz, 1H), 7.52 (d, J=9.2 Hz, 1H), 3.88 (s, 3H); MS (ESI) m/z [M+H]⁺ calcd. for C₁₁H₉IN₄, 325.0; found, 325.1.

### Step 2. Preparation of 6-(1-methyl-1H-pyrazol-4-yl)-3-(4,4,5,5-tetramethyl-1,3,2-dioxaborolan-2-yl)pyrazolo[1,5-a]pyridine (Compound S57)

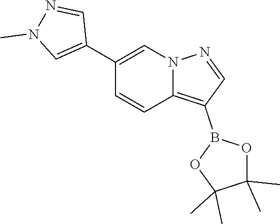

To a solution of 3-iodo-6-(1-methyl-1H-pyrazol-4-yl)pyrazolo[1,5-a]pyridine (9.0 g, 27.8 mmol, as prepared in the previous step) in THF (180 mL) was treated with 2-isopropoxy-4,4,5,5-tetramethyl-1,3,2-dioxaborolane (17.1 g, 91.7 mmol) for 5 min at 0° C. under nitrogen followed by the addition of a 1.3M solution of iPrMgCl·LiCl, in THF (31.9 mL, 41.7 mmol) dropwise at 0° C. The resulting mixture was stirred for 3 h at 0° C. under nitrogen. The reaction mixture was diluted with DCM (160 mL) then filtered through a pad of silica gel. The pad was washed with EtOAc (100 mL) and the combined filtrates were concentrated under reduced pressure to afford 5.0 g (42%) of 6-(1-methyl-1H-pyrazol-4-yl)-3-(4,4,5,5-tetramethyl-1,3,2-dioxaborolan-2-yl)pyrazolo[1,5-a]pyridine (Compound S57) as a grey white solid. ¹H NMR (400 MHZ, DMSO-d₆) δ (ppm) 9.08 (s, 1H), 8.26 (s, 1H), 8.12 (s, 1H), 8.01 (d, J=0.8 Hz, 1H), 7.82 (d, J=9.1 Hz, 1H), 7.65 (d, J=9.1 Hz, 1H), 3.89 (s, 3H), 1.32 (s, 12H).; MS (ESI) m/z [M+H]⁺ calcd. for C₁₇H₂₁BN₄O₂, 325.2; found, 325.2.

### Synthesis of 1-(2-chloropyrimidin-5-yl)-2-phenylethan-1-ol (Compound S58)

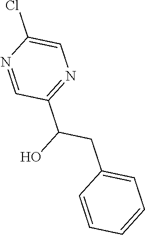 [SMILES: OC(Cc1ccccc1)c1cnc(Cl)cn1]

To a stirred solution of 2-chloropyrimidine-5-carbaldehyde (1 g, 7.02 mmol) in THF (20 mL) was added BnMgBr (1.27 g, 8.42 mmol) in portions at −78° C. under nitrogen. The resulting mixture was stirred for 1 h at −78° C. under nitrogen. The reaction was quenched with ice water then the resulting mixture was concentrated under vacuum, dissolved in water (100 mL), and extracted with EtOAc (2×100 mL). The combined organic layers were washed with brine (2×5 mL), dried over anhydrous Na₂SO₄, and filtered. The filtrate was concentrated under reduced pressure then the residue was purified by silica gel column chromatography, eluted with PE/EtOAc (1:1) to afford 220 mg (13%) of 1-(2-chloropyrimidin-5-yl)-2-phenylethan-1-ol (Compound S58) as a light-yellow solid. ¹H NMR (400 MHZ, DMSO-d₆) δ (ppm) 8.62 (s, 2H), 7.33-7.09 (m, 5H), 5.92-5.64 (m, 1H), 4.95 (t, J=6.6 Hz, 1H), 3.06-2.93 (m, 2H). MS (ESI) m/z [M+H]⁺ calcd. for C₁₂H₁₁ClN₂O, 235.06; found, 235.07.

Using the procedures described for Compound S58 and reagents, starting materials, and conditions known to those skilled in the art, the following compounds were prepared:

| Compound | Name | Characterization |
|---|---|---|
| Compound S59 | 1-(2-chloropyrimidin-5-yl)- | ¹H NMR (400 MHz, DMSO- |
| | 3-phenylpropan-1-ol | d₆) δ (ppm) 8.75 (s, 1H), 7.34- |
| | | 7.16 (m, 5H), 5.71 (d, J = |
| | | 4.8 Hz, 1H), 4.79-4.56 (m, |
| | | 1H), 2.82-2.54 (m, 2H), |
| | | 2.08-1.91 (m, 2H); MS (ESI) |
| | | m/z [M + H]⁺ calcd. for |
| | | C₁₅H₁6N₆O, 249.1; found, |
| | | 249.3. |
| Compound S60 | 1-(2-chloropyrimidin-5-yl)- | ¹H NMR (400 MHz, DMSO- |
| | 2-methylpropan-1-ol | d₆) δ (ppm) 8.70 (s, 2H), 5.56 |
| | | (d, J = 4.6 Hz, 1H), 4.44 (dd, |
| | | J = 5.9, 4.6 Hz, 1H), 1.96- |
| | | 1.84 (m, 1H), 0.88-0.74 (m, |
| | | 6H). MS (ESI) m/z [M + H]⁺ |
| | | calcd. for C₈H₁₁ClN₂O, |
| | | 187.1; found, 187.1. |
| Compound S61 | 1-(2-chloropyrimidin-5-yl)- | ¹H NMR (400 MHz, DMSO- |
| | 2-ethylbutan-1-ol | d₆) δ (ppm) 8.71 (s, 2H), 5.50 |
| | | (d, J = 4.8 Hz, 1H), 4.65 (t, |
| | | J = 5.3 Hz, 1H), 1.49-1.46 |
| | | (m, 1H), 1.39-1.21 (m, 3H), |
| | | 1.15-1.12 (m, 1H), 0.83- |
| | | 0.78 (m, 6H). MS (ESI) m/z |
| | | [M + H]⁺ calcd. for |
| | | C₁₀H₁₅ClN₂O, 215.1; found, |
| | | 215.1. |
| Compound S62 | 1-(2-chloropyrimidin-5- | ¹H NMR (400 MHz, DMSO- |
| | yl)pentan-1-ol | d₆) δ (ppm) 8.73 (s, 2H), 5.55 |
| | | (d, J = 4.8 Hz, 1H), 4.66-4.58 |
| | | (m, 1H), 1.71-1.58 (m, 2H), |
| | | 1.38-1.17 (m, 4H), 0.86 (t, |
| | | J = 6.8 Hz, 3H). MS (ESI) |
| | | m/z [M + H]⁺ calcd. For |
| | | C₉H₁₃ClN₂O, 201.1; found, |
| | | 201.1. |

### Synthesis of 5-benzyl-3-bromo-1,2,4-thiadiazole (Compound S62)

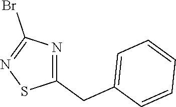 [SMILES: Brc1nsc(Cc2ccccc2)n1]

To a solution of 3-bromo-5-chloro-1,2,4-thiadiazole (500 mg, 2.51 mmol) and potassium benzyltrifluoroborate (347.5 mg, 1.76 mmol) in toluene (20 mL) and H₂O (4 mL) were added tBu₃P Pd G3 (143.4 mg, 0.25 mmol), tBu₃PHBF₄ (72.7 mg, 0.25 mmol) and K₃PO₄ (1.06 g, 5.01 mmol). After stirring for 1 h at 80° C. under nitrogen atmosphere, the mixture was allowed to cool to rt. The resulting mixture was concentrated under reduced pressure. The residue was dissolved in water (100 mL), extracted with EtOAc (3×100 mL), then dried over anhydrous Na₂SO₄ and filtered. The filtrate was concentrated under reduced pressure then the residue was purified by silica gel column chromatography, eluted with PE/EtOAc (5:1) to afford 230 mg (36%) of 5-benzyl-3-bromo-1,2,4-thiadiazole (Compound S62) as a yellow solid. ¹H NMR (400 MHZ, DMSO-d₆) δ (ppm) 7.51-7.23 (m, 5H), 4.55 (s, 2H): MS (ESI) m/z [M+H]⁺ calcd. for C₉H₇BrN₂S, 254.9; found, 255.1.

### Synthesis of 2-benzyl-5-chloro-1,3,4-oxadiazole (Compound S63)

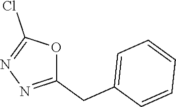 [SMILES: Clc1nnc(Cc2ccccc2)o1]

To a solution of 5-benzyl-1,3,4-oxadiazol-2-amine (100 mg, 0.57 mmol) and CuCl₂ (153.5 mg, 1.14 mmol) in ACN (5 mL) was added 2-methyl-2-propylnitrite (58.9 mg, 0.57 mmol) at rt. The mixture was stirring at 60° C. for 1 h, then mixture was allowed to cool to rt. Then resulting mixture was diluted with H₂O (15 mL). The resulting mixture was extracted with EtOAc (3×50 mL). The combined organic layers dried over anhydrous Na₂SO₄ and filtered. The filtrate was concentrated under reduced pressure then the residue was purified by Prep-TLC (PE/EtOAc 1:1) to afford 10 mg (9%) of 2-benzyl-5-chloro-1,3,4-oxadiazole (Compound S63) as a colorless oil. ¹H NMR (400 MHZ, DMSO-d₆) δ (ppm) 7.42-7.27 (m, 5H), 4.31 (s, 2H). MS (ESI) m/z [M+H]⁺ calcd. for C₉H₇ClN₂O, 195.0; found, 195.1.

### Synthesis of 2-chloro-5-(furan-2-ylmethyl)pyrimidine (Compound S64)

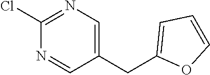 [SMILES: Clc1ncc(Cc2ccco2)cn1]

To a solution of 2-chloro-5-(chloromethyl)pyrimidine (1 g, 6.14 mmol) and tributyl(furan-2-yl)stannane (2.6 g, 7.36 mmol) in dioxane (80 mL) was added Pd(PPh₃)₄ (1.4 g, 1.23 mmol). After stirring for 16 h at 60° C. under nitrogen, the resulting mixture was concentrated under reduced pressure. The residue was purified by silica gel column chromatography, eluted with PE/EtOAc (1:1) to afford 770 mg (64%) of 2-chloro-5-(furan-2-ylmethyl)pyrimidine (Compound S64) as a white solid. ¹H NMR (400 MHZ, DMSO-d₆) δ 8.73 (s, 1H), 7.61 (d, J=1.9 Hz, 1H), 6.42 (d, J=3.3 Hz, 1H), 6.22 (d, J=3.2 Hz, 1H). MS (ESI) m/z [M+H]⁺ calcd. for C₉H₇ClN₂O, 195.0; found 195.0.

### Synthesis of 2-chloro-5-(furan-3-ylmethyl)pyrimidine (Compound S65)

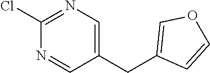 [SMILES: Clc1ncc(Cc2ccoc2)cn1]

To a solution of 2-chloro-5-(chloromethyl)pyrimidine (500 mg, 3.07 mmol) and 2-(furan-3-yl)-4,4,5,5-tetramethyl-1,3,2-dioxaborolane (714.3 mg, 3.68 mmol) in dioxane (5 mL) and H₂O (0.5 mL) were added Pd(PPh₃)₂Cl₂ (430.6 mg, 0.61 mmol) and K₃PO₄ (1302.2 mg, 6.13 mmol). After stirring for 16 h at 90° C. under nitrogen, the resulting mixture was concentrated under reduced pressure and extracted with EtOAc (3×30 mL). The combined organic layers were washed with brine (10 mL), dried over anhydrous Na₂SO₄. After filtration, the filtrate was concentrated under reduced pressure. The residue was purified by silica gel column chromatography, eluted with DCM/MeOH (10:1) to afford 100 mg (17%) of 2-chloro-5-(furan-3-ylmethyl)pyrimidine (Compound S65) as a light yellow solid. ¹H NMR (400 MHZ, DMSO-d₆) δ (ppm) 8.69 (s, 2H), 7.57 (d, J=4.0 Hz, 2H), 6.47-6.42 (m, 1H), 3.80 (s, 2H); MS (ESI) m/z [M+H]⁺ calcd. for C₉H₇ClN₂O, 195.0 found, 195.1.

### Synthesis of 2-((2-chloropyrimidin-5-yl)methyl)oxazole (Compound S66)

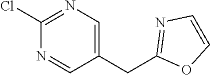 [SMILES: Clc1ncc(Cc2ncco2)cn1]

To a stirred solution of 2-(chloromethyl)oxazole (500 mg, 4.25 mmol) and (2-chloropyrimidin-5-yl) boronic acid (1.01 g, 6.38 mmol) in dioxane (10 mL) and H₂O (1 mL) were added Pd-PEPPSI-IPent^{Cl} 2-methylpyridine (357.8 mg, 0.43 mmol) and K₃PO₄ (1.81 g, 8.51 mmol) at rt. The reaction was stirred overnight at 90° C. under nitrogen. The resulting mixture was filtered, and the filter cake was washed with DCM (3×50 mL). The filtrate was concentrated under reduced pressure. The residue was purified by silica gel column chromatography, eluted with PE/EtOAc (1:1) to afford 140 mg (17%) of 2-((2-chloropyrimidin-5-yl)methyl)oxazole (Compound S66) as a yellow solid. ¹H NMR (400 MHZ, DMSO-d₆) δ (ppm) 8.79 (s, 2H), 8.08 (d, J=0.9 Hz, 1H), 7.16 (d, J=0.9 Hz, 1H), 4.28 (s, 2H); MS (ESI) m/z [M+H]⁺ calcd. for C₈H₆ClN₃O, 196.0; found, 196.1.

### Synthesis of 4-((2-chloropyrimidin-5-yl)methyl)oxazole (Compound S67)

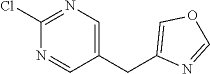 [SMILES: Clc1ncc(Cc2cocn2)cn1]

To a solution of 2-chloropyrimidin-5-ylboronic acid (300 mg, 1.90 mmol) and 4-(bromomethyl)-1,3-oxazole (306.9 mg, 1.90 mmol) in dioxane (20 mL) and H₂O (2 mL) were added K₃PO₄ (804.3 mg, 3.79 mmol) and Pd(dppf)Cl₂·DCM (154.3 mg, 0.19 mmol). After stirring 16 h at 90° C. under nitrogen, the mixture was cooled to rt and concentrated under reduced pressure. The residue was dissolved in water (100 mL). The aqueous layer was extracted with DCM (3×100 mL). The resulting mixture was concentrated under reduced pressure. The residue was purified by Prep-TLC DCM/MeOH (10:1) to afford 180 mg (49%) of 4-((2-chloropyrimidin-5-yl)methyl)oxazole (Compound S67) as a brown oil. ¹H NMR (400 MHz, DMSO-d₆) δ (ppm) 8.72 (s, 2H), 8.34 (s, 1H), 7.95 (d, J=1.2 Hz, 1H), 3.93 (s, 2H). MS (ESI) m/z [M+H]⁺ calcd. for C₈H₆ClN₃O, 196.0; found 196.0.

### Synthesis of 4-((2-chloropyrimidin-5-yl)methyl) thiazole (Compound S68)

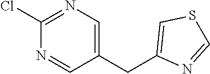 [SMILES: Clc1ncc(Cc2cscn2)cn1]

To a solution of 2-chloropyrimidin-5-ylboronic acid (500 mg, 3.16 mmol) and 4-(chloromethyl) thiazole hydrochloride (506.2 mg, 3.79 mmol) in dioxane (25 mL) and water (25 mL) were added K₃PO₄ (1.34 g, 6.32 mmol) and Pd-PEPPSI-IPent^{Cl} 2-methylpyridine (531.2 mg, 0.63 mmol). After stirring for 16 h at 90° C. under nitrogen, the resulting mixture was concentrated under reduced pressure and extracted with EtOAc (3×50 mL). The combined organic layers were washed with brine (10 mL) and dried over anhydrous Na₂SO₄. After filtration, the filtrate was concentrated under reduced pressure. The residue was purified by silica gel column chromatography, eluted with DCM/MeOH (10:1) to afford 220 mg (33%) of 4-((2-chloropyrimidin-5-yl)methyl) thiazole (Compound S68) as a yellow solid. ¹H NMR (400 MHZ, DMSO-d₆) δ (ppm) 9.08 (d, J=2.0 Hz, 1H), 8.73 (s, 2H), 7.52-7.47 (m, 1H), 4.19 (s, 2H); MS (ESI) m/z [M+H]⁺ calcd. for C₈H₆ClN₃S, 212.0; found, 212.1.

### Synthesis of 2-chloro-5-(thiophen-2-ylmethyl)pyrimidine (Compound S69)

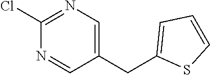 [SMILES: Clc1ncc(Cc2cccs2)cn1]

To a mixture of 2-chloropyrimidin-5-ylboronic acid (800 mg, 5.052 mmol) and 2-(chloromethyl) thiophene (1.00 g, 7.58 mmol) in dioxane (20 mL) and H₂O (2 mL) was added K₃PO₄ (3.22 g, 15.16 mmol) and (PPh₃)₂PdCl₂ (354.6 mg, 0.505 mmol). The reaction was stirred for 2 h at 90° C. under nitrogen. To the mixture was added H₂O (5 mL) and extracted with EtOAc (3×50 mL). The combined organic layers were washed with brine (10 mL) and dried over anhydrous Na₂SO₄. After filtration, the filtrate was concentrated under reduced pressure. The residue was purified by Prep-TLC PE/EtOAc (1:1) to afford 400 mg (38%) of 2-chloro-5-(thiophen-2-ylmethyl)pyrimidine (Compound S69) as a white solid. ¹H NMR (400 MHz, DMSO-d₆) δ (ppm) 8.73 (s, 2H), 7.40 (m, 1H), 7.01-6.92 (m, 2H), 4.23 (s, 2H). MS (ESI) m/z [M+H]⁺ calcd. for C₉H₇ClN₂S, 211.0; found, 211.1.

### Synthesis of 2-chloro-5-(pyridin-4-ylmethyl)pyrimidine (Compound S70)

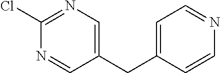 [SMILES: Clc1ncc(Cc2ccncc2)cn1]

To a stirred solution of 4-(bromomethyl)pyridine hydrobromide (3.19 g, 12.63 mmol) and (2-chloropyrimidin-5-yl) boronic acid (2 g, 12.63 mmol) in dioxane (60 mL) and H₂O (6 mL) were added Dichlorobis(di-tert-butylphenylphosphine) palladium (II) (788.1 mg, 1.26 mmol) and K₃PO₄ (8.04 g, 37.89 mmol) at rt. The reaction was stirred overnight at 90° C. under nitrogen. The resulting mixture was filtered, the filter cake was washed with DCM (3×100 mL). The filtrate was concentrated under reduced pressure. The residue was purified by silica gel column chromatography, eluted with DCM/MeOH (15:1) to afford 150 mg (6%) of 2-chloro-5-(pyridin-4-ylmethyl)pyrimidine (Compound S70) as a brown solid. ¹H NMR (400 MHz, DMSO-d₆) δ ppm 8.75 (s, 2H), 8.53-8.47 (m, 2H), 7.33-7.29 (m, 2H), 4.05 (s, 2H); MS (ESI) m/z [M+H]⁺ calcd. for C₁₀H₈ClN₃, 206.0; found, 206.1.

### Synthesis of 2-chloro-5-(1,3,4-oxadiazol-2-ylmethyl)pyrimidine (Compound S71)

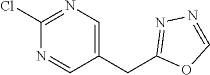 [SMILES: Clc1ncc(Cc2nnco2)cn1]

To a stirred solution of 2-chloropyrimidin-5-ylboronic acid (500 mg, 3.16 mmol) and 2-(chloromethyl)-1,3,4-oxadiazole (561.4 mg, 4.74 mmol) in dioxane (20 mL) were added K₃PO₄ (2.01 g, 9.4740 mmol), Pd-PEPPSI-IPent^{Ct} 2-methylpyridine (26.6 mg, 0.032 mmol) and water (2 mL) in portions at rt under nitrogen. The reaction was stirred overnight at 90° C. under nitrogen. The resulting mixture was filtered, and the filter cake was washed with dioxane (2×10 mL). The filtrate was concentrated under reduced pressure. The residue was purified by reversed-phase flash chromatography with C18 silica gel column, eluted with ACN in Water (10 mM NH₄HCO₃), 5% to 50% gradient to afford 170 mg (27%) of 2-chloro-5-(1,3,4-oxadiazol-2-ylmethyl)pyrimidine (Compound S71) as a light red solid. ¹H NMR (400 MHZ, DMSO-d₆) δ (ppm) 9.22 (s, 1H), 8.83 (s, 2H), 4.42 (s, 2H). MS (ESI) m/z [M+H]⁺ calcd. for C₇H₅ClN₄O, 197.0; found, 197.1.

### Synthesis of 2-((2-chloropyrimidin-5-yl)methyl)thiazole (Compound S72)

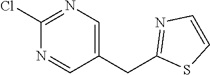 [SMILES: Clc1ncc(Cc2nccs2)cn1]

To a solution of 2-chloro-5-(chloromethyl)pyrimidine (350 mg, 2.15 mmol) and Pd(PPh₃)₄ (496 mg, 0.43 mmol) in dioxane (5.0 mL) was added 2-(tributylstannyl)-1,3-thiazole (844 mg, 2.26 mmol). The reaction was stirred for 16 h at 60° C. under nitrogen, then quenched by the addition of sat. aq. KF (1.0 mL). The resulting mixture was concentrated under reduced pressure. The residue was purified by silica gel column chromatography, eluted with PE/EtOAc (2:1) to afford 120 mg (24%) of 2-((2-chloropyrimidin-5-yl)methyl) thiazole (Compound S72) as a yellow oil. ¹H NMR (400 MHZ, DMSO-d₆) δ (ppm) 8.81 (s, 2H), 7.75 (d, J=3.3 Hz, 1H), 7.66 (d, J=3.3 Hz, 1H), 4.47 (s, 2H).; MS (ESI) m/z [M+H]⁺ calcd. for C₈H₆ClN₃S, 212.0; found, 212.1.

### Synthesis of 2-chloro-4-(chloromethyl)thiophene (Compound S73)

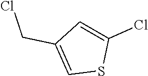

To a stirred solution of (5-chlorothiophen-3-yl)methanol (300 mg, 2.02 mmol) and thionyl chloride (5 mL) at 0° C. The reaction was stirred for 1 h at 0° C., then concentrated under reduced pressure. The crude 2-chloro-4-(chloromethyl)thiophene (Compound S73) was used directly without further purification.

### Synthesis of 3-((2-chloropyrimidin-5-yl)methyl)-1H-indole (Compound S74)

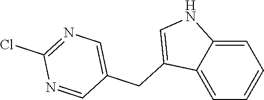 [SMILES: Clc1ncc(Cc2c[nH]c3ccccc23)cn1]

To a stirred solution of indole (1.08 g, 9.20 mmol) in THF (90 mL) was added NaH (552.1 mg, 13.80 mmol, 60% oil dispersion), stirred for 15 min at rt then the mixture was cooled to 0° C., and 2-chloro-5-(chloromethyl)pyrimidine (1.5 g, 9.20 mmol) was added at 0° C. under nitrogen. The reaction was warmed to rt, stirred for 1 h, and then cooled back to 0° C., and quenched by the addition of water (100 mL). The aqueous layer was extracted with EtOAc (4×100 mL). Then resulting mixture was concentrated under reduced pressure. The residue was purified by silica gel column chromatography, eluted with PE/EtOAc (6:1) to afford 251 mg (11%) of 3-[(2-chloropyrimidin-5-yl)methyl]indole (Compound S74) as an off-white solid. ¹H NMR (400 MHZ, DMSO-d₆) δ (ppm) 10.98 (s, 1H), 8.73 (s, 2H), 7.55-7.46 (m, 1H), 7.36 (d, J=8.1 Hz, 1H), 7.23 (d, J=2.4 Hz, 1H), 7.23-7.08 (m, 1H), 7.07-6.97 (m, 1H), 4.09 (s, 2H): MS (ESI) m/z [M+H]⁺ calcd. for C₁₃H₁₀ClN₃, 244.0; found 244.0.

### Synthesis of 1-((2-chloropyrimidin-5-yl)methyl)indoline (Compound S75)

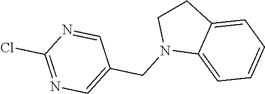 [SMILES: Clc1ncc(CN2CCc3ccccc32)cn1]

To a solution of 2-chloro-5-(chloromethyl)pyrimidine (1 g, 6.14 mmol) and indoline (0.88 g, 7.36 mmol) in DMF (20 mL) was added K₂CO₃ (2.12 g, 15.34 mmol) in portions at 0° C. under nitrogen. The reaction was stirred overnight at rt, then the mixture was washed with water (3×30 mL). The aqueous layer was extracted with EtOAc (3×50 mL). The resulting mixture was concentrated under reduced pressure. The residue was purified by silica gel column chromatography, eluted with PE/EtOAc (3:1) to afford 1.28 g (76%) of 1-((2-chloropyrimidin-5-yl)methyl)indoline (Compound S75) as a white solid. ¹H NMR (400 MHz, DMSO-d₆) δ (ppm) 8.79 (s, 2H), 7.13-6.96 (m, 2H), 6.72-6.55 (m, 2H), 4.34 (s, 2H), 3.28 (t, J=8.3 Hz, 2H), 2.97 (t, J=8.3 Hz, 2H). MS (ESI) m/z [M+H]⁺ calcd. for C₁₃H₁₂ClN₃, 246.0; found 246.0.

### Synthesis of 4-phenethylcyclohex-1-en-1-yl trifluoromethanesulfonate (Compound S76)

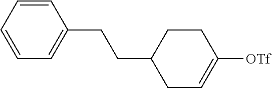 [SMILES: O=S(=O)(OC1=CCC(CCc2ccccc2)CC1)C(F)(F)F]

### Step 1. Preparation of phenethyltriphenylphosphonium Iodide

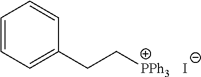

To a solution of (2-iodoethyl)benzene (10 g, 43.09 mmol) in xylene (100 mL) was added PPh₃ (11.3 g, 43.09 mmol). The reaction was stirred overnight at 80° C. under nitrogen. The mixture was allowed to cool to rt. Then the residue was purified by silica gel column chromatography eluting with DCM/MeOH (20:1) to afford 12 g (76%) of triphenyl(2-phenylethyl)phosphonium iodide as a white solid. ¹H NMR (400 MHZ, DMSO-d₆) δ (ppm) 7.97-7.73 (m, 15H), 7.33-7.21 (m, 5H), 3.96 (m, 2H), 2.95-2.84 (m, 2H); MS (ESI) m/z [M+H]⁺ calcd. for C₂₆H₂₄P⁺, 367.2; found, 367.3.

### Step 2. Preparation of 8-(2-phenylethylidene)-1,4-dioxaspiro[4.5]decane

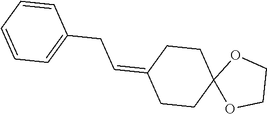 [SMILES: C(Cc1ccccc1)=C1CCC2(CC1)OCCO2]

To a solution of triphenyl(2-phenylethyl)phosphonium iodide (11 g, 29.94 mmol, as prepared in the previous step) and 1,4-dioxaspiro[4.5]decan-8-one (5.61 g, 35.92 mmol) in THF (150 mL) was added NaH (2.16 g, 89.81 mmol, 60% oil dispersion). The reaction was stirred overnight at rt under nitrogen. Then reaction was quenched by the addition of H₂O (20 mL) at 0° C. The resulting mixture was extracted with EtOAc (3×200 mL). The residue was purified by silica gel column chromatography eluting with PE/EtOAc (10:1) to afford 1.5 g (21%) of 8-(2-phenylethylidene)-1,4-dioxaspiro[4.5]decane as a yellow oil. ¹H NMR (400 MHz, DMSO-d₆) δ (ppm) 7.32-7.18 (m, 5H), 5.31 (t, J=7.5 Hz, 1H), 3.89 (d, J=3.0 Hz, 4H), 3.33 (m, 2H), 2.32 (t, J=6.6 Hz, 2H), 2.19 (t, J=6.5 Hz, 2H), 1.64-1.56 (m, 4H); MS (ESI) m/z [M+H]⁺ calcd. for C₁₆H₂₀O₂, 245.1; found, 245.2.

### Step 3. Preparation of 8-phenethyl-1,4-dioxaspiro[4.5]decane

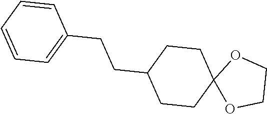 [SMILES: c1ccc(CCC2CCC3(CC2)OCCO3)cc1]

To a solution of 8-(2-phenylethylidene)-1,4-dioxaspiro[4.5]decane (1.5 g, 6.14 mmol, as prepared in the previous step) dissolved in MeOH (20 mL) was added 10% Pd/C (0.65 g, 6.14 mmol). The reaction was stirred for 6 h at rt under a hydrogen atmosphere. The resulting mixture was filtered, then filter cake was washed with MeOH (5 mL). The filtrate was concentrated under reduced pressure to afford 1.3 g (86%) of 8-phenylethyl-1,4-dioxaspiro[4.5]decane as a yellow oil. ¹H NMR (400 MHZ, DMSO-d₆) δ (ppm) 7.27-7.11 (m, 5H), 3.84 (m, 4H), 2.62-2.54 (m, 2H), 1.68 (m, 4H), 1.53-1.35 (m, 4H), 1.20 (m, 3H); MS (ESI) m/z [M+H]⁺ calcd. for C₁₈H₂₂O₂, 247.2; found, 247.1.

### Step 4. Preparation of 4-phenethylcyclohexan-1-one

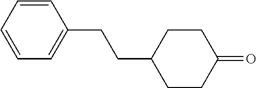 [SMILES: O=C1CCC(CCc2ccccc2)CC1]

To a solution of 8-phenylethyl-1,4-dioxaspiro[4.5]decane (1.3 g, 5.28 mmol, as prepared in the previous step) in THF (10 mL) was added 2M HCl (5 mL). The reaction was stirred for 6 h at rt. Then resulting mixture was extracted with EtOAc (2×30 mL). The residue was purified by silica gel column chromatography, eluted with PE/EtOAc (8:1) to afford 1 g (94%) of 4-phenethylcyclohexan-1-one as a yellow oil. ¹H NMR (400 MHZ, DMSO-d₆) δ (ppm) 7.33-7.13 (m, 5H), 2.68-2.60 (m, 2H), 2.37-2.32 (m, 2H), 2.19 (m, 2H), 2.07-1.98 (m, 2H), 1.71 (m, 1H), 1.58 (m, 2H), 1.37 (m, 2H); MS (ESI) m/z [M+H]⁺ calcd. for C₁₄H₁₈O, 203.1; found, 203.2.

### Step 5. Preparation of 4-phenethylcyclohex-1-en-1-yl trifluoromethanesulfonate (Compound S76)

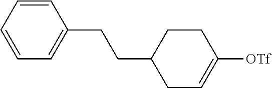 [SMILES: O=S(=O)(OC1=CCC(CCc2ccccc2)CC1)C(F)(F)F]

To a solution of 4-(2-phenylethyl)cyclohexan-1-one (1 g, 4.94 mmol, as prepared in the previous step) and 2,6-di-tert-butyl-4-methylpyridine (2.03 g, 9.89 mmol) in DCM (20 mL) was added Tf₂O (2.79 g, 9.886 mmol). The reaction was stirred for 3 h at rt. The reaction was quenched by the addition of MeOH (5 mL) at 0° C. The resulting mixture was added H₂O (10 mL) and extracted with DCM (2×50 mL). The residue was purified by silica gel column chromatography, eluted with PE/EtOAc (10:1) to afford 1 g (61%) of 4-phenethylcyclohex-1-en-1-yl trifluoromethanesulfonate (Compound S76) as a yellow oil. ¹H NMR (400 MHZ, DMSO-d₆) δ (ppm) 7.28-7.19 (m, 5H), 5.87 (m, 1H), 2.62 (s, 2H), 1.91 (m, 3H), 1.60-1.52 (m, 2H), 1.44 (m, 4H); MS (ESI) m/z [M−H]⁺ calcd. for C₁₅H₁₇F₃O₃S, 333.1; found, 333.2.

### Synthesis of 4-(phenoxymethyl)cyclohex-1-en-1-yl trifluoromethanesulfonate (Compound S77)

### Step 1. Synthesis of (4-oxocyclohexyl)methyl 4-methylbenzenesulfonate

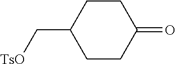 [SMILES: Cc1ccc(S(=O)(=O)OCC2CCC(=O)CC2)cc1]

To a stirred solution of 4-(hydroxymethyl)cyclohexan-1-one (3 g, 23.41 mmol) in pyridine (30 mL) was added TsCl (5.35 g, 28.09 mmol) in portions at 0° C. under air atmosphere. The reaction was stirred overnight at rt. The resulting mixture was concentrated under vacuum. The residue was dissolved in water (200 mL), then was extracted with DCM (2×200 mL). The combined organic layers were washed with brine (2×10 mL), dried over anhydrous Na₂SO₄, and filtered. The filtrate was concentrated under reduced pressure then the residue was purified by silica gel column chromatography eluting with PE/EtOAc (1:1) to afford 2.6 g (39%) of (4-oxocyclohexyl)methyl 4-methylbenzenesulfonate as a light yellow oil. ¹H NMR (400 MHZ, DMSO-d₆) δ (ppm) 7.86-7.77 (m, 2H), 7.58-7.40 (m, 2H), 3.97 (d, J=6.4 Hz, 2H), 2.43 (s, 3H), 2.38-2.30 (m, 2H), 2.21-2.03 (m, 3H), 1.87-1.90 (m, 2H), 1.43-1.22 (m, 2H). MS (ESI) m/z [M+H]⁺ calcd. for C₁₄H₁₈O₄S, 283.09; found, 283.11.

Step 2. Synthesis of 4-(phenoxymethyl)cyclohexan-1-one

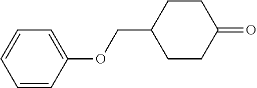 [SMILES: O=C1CCC(COc2ccccc2)CC1]

To a stirred solution of (4-oxocyclohexyl)methyl 4-methylbenzenesulfonate (2.5 g, 8.85 mmol, as prepared in the previous step) and phenol (1.67 g, 17.71 mmol) in DMF (40 mL) was added Cs₂CO₃ (5.77 g, 17.71 mmol) in portions at rt. The reaction was stirred for 3 h at 80° C. The resulting mixture was concentrated under vacuum. The residue was dissolved in H₂O (200 mL), then was extracted with EtOAc (2×200 mL). The combined organic layers were washed with brine (2×10 mL), dried over anhydrous Na₂SO₄, and filtered. The filtrate was concentrated under reduced pressure then the residue was purified by silica gel column chromatography, eluted with PE/EtOAc (1:1) to afford 1.5 g (83%) of 4-(phenoxymethyl)cyclohexan-1-one as an off-white solid. ¹H NMR (400 MHZ, DMSO-d₆) δ (ppm) 7.30-7.26 (m, 2H), 6.99-6.88 (m, 3H), 3.90 (d, J=6.4 Hz, 2H), 2.50-2.39 (m, 2H), 2.32-2.22 (m, 3H), 2.11-2.01 (m, 2H), 1.60-1.42 (m, 2H). MS (ESI) m/z [M+H]⁺ calcd. for C₁₃H₁₆O₂, 205.1; found, 205.2.

### Step 3. Synthesis of 4-(phenoxymethyl)cyclohex-1-en-1-yl trifluoromethanesulfonate (Compound S77)

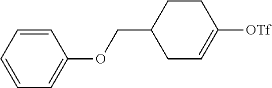 [SMILES: O=S(=O)(OC1=CCC(COc2ccccc2)CC1)C(F)(F)F]

To a stirred solution of 4-(phenoxymethyl)cyclohexan-1-one (1 g, 4.89 mmol, as prepared in the previous step) in DCM (10 mL) was added Tf₂O (2.07 g, 7.34 mmol) dropwise at 0° C. The resulting mixture was stirred for 1 h at rt, then concentrated under vacuum. The residue was dissolved in water (100 mL), then was extracted with DCM (2×100 mL). The combined organic layers were washed with brine (2×5 mL), dried over anhydrous Na₂SO₄, and filtered. The filtrate was concentrated under reduced pressure then the residue was purified by reversed-phase flash chromatography to afford 1.2 g (73%) of 4-(phenoxymethyl)cyclohex-1-en-1-yl trifluoromethanesulfonate (Compound S77) as a white solid. ¹H NMR (400 MHZ, DMSO-d₆) δ (ppm) 7.32-7.22 (m, 2H), 6.98-6.87 (m, 3H), 6.01-5.81 (m, 1H), 3.93-3.81 (m, 2H), 2.49-2.21 (m, 3H), 2.12-1.92 (m, 3H), 1.65-1.42 (m, 1H). MS (ESI) m/z [M+H]⁺ calcd. for C₁₄H₁₅F₃O₄S, 337.06; found, 337.16.

### Synthesis of 2-(chloromethyl)-5-methylfuran (Compound S78)

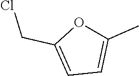

To a solution of (5-methylfuran-2-yl)methanol (100 mg, 0.89 mmol) and NEt₃ (90.3 mg, 0.89 mmol) in DCM (10 mL) was added SOCl₂ (137.9 mg, 1.16 mmol) at 0° C. The mixture was stirred at 0° C. for 0.5 h. Then reaction was quenched by the addition of water (1 mL) at 0° C. The resulting mixture was extracted with DCM (3×10 mL), dried over anhydrous Na₂SO₄, and filtered. The filtrate was concentrated under reduced pressure to afford 60 mg (21%) of 2-(chloromethyl)-5-methylfuran (Compound S78) as a yellow crude oil which was used without further purification.

### Synthesis of tert-butyl 4-(5-benzoyloxazol-2-yl)piperazine-1-carboxylate (Compound S79)

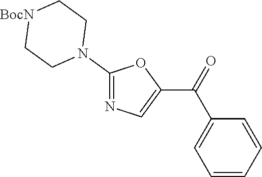 [SMILES: CC(C)(C)OC(=O)N1CCN(c2ncc(C(=O)c3ccccc3)o2)CC1]

### Step 1. Preparation of ethyl 2-(4-(tert-butoxycarbonyl)piperazin-1-yl)oxazole-5-carboxylate

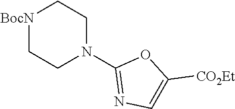 [SMILES: CCOC(=O)c1cnc(N2CCN(C(=O)OC(C)(C)C)CC2)o1]

To a microwave vessel equipped with a magnetic stir bar was added ethyl 2-chloro-1,3-oxazole-5-carboxylate (1.92 g, 10.9 mmol), tert-butyl 1-piperazinecarboxylate (2.45 g, 13.1 mmol), dioxane (25.6 mL), and NEt₃ (4.58 mL, 32.8 mmol), then the vessel was capped and heated to 120° C. for 1 h. The mixture was concentrated under reduced pressure affording crude product. This reaction was repeated twice more using 2.50 g and 2.32 g of ethyl 2-chloro-1,3-oxazole-5-carboxylate and the crude products were combined and purified by silica gel column chromatography eluting with 0-70% acetone/hexanes to afford 10.54 g (84%) of ethyl 2-(4-(tert-butoxycarbonyl)piperazin-1-yl)oxazole-5-carboxylate as a white solid. ¹H NMR (400 MHZ, CDCl₃) δ (ppm) 7.53 (s, 1H), 4.32 (q, J=7.1 Hz, 2H), 3.65-3.57 (m, 4H), 3.56-3.49 (m, 4H), 1.48 (s, 9H), 1.35 (t, J=7.1 Hz, 3H); MS (ESI) m/z [M+H]⁺ calcd. for C₁₅H₂₃N₃O₅: 326.2, Found: 326.1; HPLC purity: 210 nm: 100.0%; 254 nm: 100.0%.

### Step 2. Preparation of 2-(4-(tert-butoxycarbonyl)piperazin-1-yl)oxazole-5-carboxylic acid

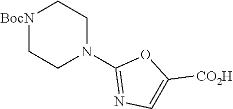 [SMILES: CC(C)(C)OC(=O)N1CCN(c2ncc(C(=O)O)o2)CC1]

To a 250 mL round bottom flask equipped with a magnetic stir bar were added tert-butyl 4-[5-(ethoxycarbonyl)-1,3-oxazol-2-yl]piperazine-1-carboxylate (10.54 g, 32.39 mmol, as prepared in the previous step), THF (230 mL) and MeOH (52.5 mL). 1.00 M aqueous NaOH solution (52.70 mL, 52.70 mmol) was added then the mixture was stirred at rt for 2 h. The volatiles were removed under reduced pressure then the aqueous mixture was acidified tp pH 3 with 1M HCl. The aqueous suspension was extracted with 1:1 EtOAc: THF (440 mL) then (3×300 mL). The combined organics were washed with brine, dried over anhydrous NazSO₄, and filtered. The filtrate was concentrated under reduced pressure to afford 9.68 g (100%) of 2-(4-(tert-butoxycarbonyl)piperazin-1-yl)oxazole-5-carboxylic acid as a white solid. ¹H NMR (400 MHZ, DMSO-d₆) δ (ppm) 12.80 (br s, 1H), 7.61 (s, 1H), 3.55-3.37 (m, 8H), 1.42 (s, 9H); MS (ESI) m/z [M+H]⁺ calcd. for C₁₃H₁₉N₃O₅: 298.1, Found: 298.0; HPLC purity: 210 nm: 100.0%; 254 nm: 100.0%.

### Step 3. Preparation of tert-butyl 4-(5-(methoxy(methyl)carbamoyl) oxazol-2-yl)piperazine-1-carboxylate

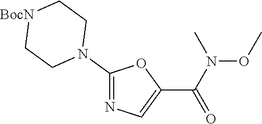 [SMILES: CON(C)C(=O)c1cnc(N2CCN(C(=O)OC(C)(C)C)CC2)o1]

To a 1 L round bottom flask equipped with a magnetic stir bar was added 2-[4-(tert-butoxycarbonyl)piperazin-1-yl]-1,3-oxazole-5-carboxylic acid (9.68 g, 32.6 mmol, as prepared in the previous step) then DCM (220 mL) was added. The mixture was cooled to 0° C., and HOBt (6.446 g, 32.83 mmol), N,O-dimethylhydroxylamine hydrochloride (3.203 g, 32.83 mmol), and DIPEA (5.719 mL, 32.83 mmol) were added. EDCl (6.294 g, 32.83 mmol) was added and the mixture was stirred at 0° C. for 15 min, then warmed to rt and stirred for 6.5 h. Additional EDCI (1.560 g, 8.140 mmol), N,O-dimethylhydroxylamine hydrochloride (1.60 g, 16.4 mmol), and DIPEA (2.836 mL, 16.28 mmol) were added and the mixture was stirred at rt overnight. The reaction was diluted with DCM, washed with sat. NaHCO₃ solution (2×), water, and brine, then the organic layer was dried over anhydrous MgSO₄ and filtered. The filtrate was concentrated under reduced pressure afford 10.78 to g (97%) of tert-butyl 4-(5-(methoxy(methyl)carbamoyl)oxazol-2-yl)piperazine-1-carboxylate as a white solid. ¹H NMR (400 MHZ, CDCl₃) δ (ppm) 7.49 (s, 1H), 3.74 (s, 3H), 3.66-3.58 (m, 4H), 3.57-3.49 (m, 4H), 3.30 (s, 3H), 1.49 (s, 9H); MS (ESI) m/z [M+H]⁺ calcd. for C₁₅H₂₄N₄O₅: 341.2, Found: 341.1; HPLC purity: 210 nm: 96.2%; 254 nm: 100.0%.

### Step 4. Preparation of tert-butyl 4-(5-benzoyloxazol-2-yl)piperazine-1-carboxylate (Compound S79)

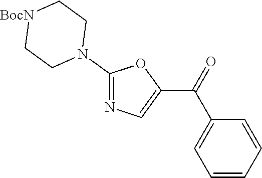 [SMILES: CC(C)(C)OC(=O)N1CCN(c2ncc(C(=O)c3ccccc3)o2)CC1]

To a 1 L round bottom flask equipped with a magnetic stir bar was added tert-butyl 4-{5-[methoxy(methyl)carbamoyl]-1,3-oxazol-2-yl}piperazine-1-carboxylate (10.7 g, 31.4 mmol, as prepared in the previous step) and anhydrous THF (87.9 mL). The mixture was cooled to −10° C., then 1.0M solution of PhMgBr in THF (70.7 mL, 70.7 mmol) was added dropwise over 30 min maintaining the low temperature. The reaction was stirred at −10° C. for 4.5 h, then quenched by the dropwise addition of sat. aq. NH₄Cl solution. The mixture was warmed to rt, diluted with EtOAc (600 mL), and water was added to dissolve any solids. The layers were separated and the aqueous layer was extracted with EtOAc (2×100 mL), then the organic layers were combined, dried over anhydrous Na₂SO₄, and filtered. The filtrate was concentrated under reduced pressure, then the residue was purified by silica gel chromatography eluted with 0-100% EtOAc/hexane to afford 10.13 g (90%) of tert-butyl 4-(5-benzoyloxazol-2-yl)piperazine-1-carboxylate (Compound S79) as a white solid. ¹H NMR (400 MHZ, CDCl₃) δ (ppm) 7.87-7.79 (m, 2H), 7.62-7.56 (m, 1H), 7.55 (s, 1H), 7.53-7.46 (m, 2H), 3.76-3.67 (m, 4H), 3.62-3.54 (m, 4H), 1.50 (s, 9H); MS (ESI) m/z [M+H]⁺ calcd. for C₁₉H₂₃N₃O₄: 358.2, Found: 358.1; HPLC purity: 210 nm: 100.0%; 254 nm: 100.0%.

### Synthesis of 2-Chloro-5-(pyridin-2-ylmethyl)pyrimidine (Compound S80)

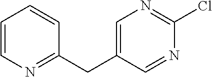 [SMILES: Clc1ncc(Cc2ccccn2)cn1]

### Step 1. Preparation of Potassium Pyridine-2-ylacetate

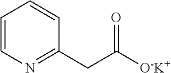

In dried glassware, a solution of methyl 2-pyridylacetate (0.500 g, 3.31 mmol), absolute ethanol (6.6 mL), and water (0.63 mL, 3.47 mmol) was heated at 60° C. A solution of potassium tert-butoxide (0.390 g, 3.47 mmol) was prepared in absolute ethanol (3.3 mL) in dried glassware, and then added dropwise to the initial solution at 60° C. over 30 min. After complete conversion, the solvent was removed under vacuum and the residue was stirred in anhydrous Et₂O (6.6 mL) for 30 min. The solids were filtered through a frit and washed in quick sequence with ethanol/ether (1:1, 0.6 mL×2) and Et₂O (3 mL×3). The white solid was transferred to a dried flask and placed under vacuum in a 30° C. bath for 2 h to afford 468 mg (81%) of potassium pyridine-2-ylacetate as a white solid. ¹H NMR (400 MHZ, DMSO-d₆) δ (ppm) 8.33 (d, J=4.3 Hz, 1H) 7.57 (td, J=7.6, 1.8, 1H) 7.27 (d, J=7.8 Hz, 1H) 7.06 (dd, J=6.6, 5.4 Hz 1H) 3.32 (s, 2H); ¹³C NMR (100 MHZ, DMSO-d₆) δ (ppm) 49.5, 119.9, 123.8, 135.1, 147.8, 160.5, 171.5; MS (ESI) m/z [M+H]⁺ calcd. for C₇H₈NO₂: 138.1; found, 138.0.

### Step 2. 2-Chloro-5-(pyridin-2-ylmethyl)pyrimidine (Compound S80)

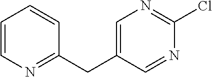 [SMILES: Clc1ncc(Cc2ccccn2)cn1]

5-Bromo-2-chloropyrimidine (110 mg, 0.590 mmol), Xantphos (22 mg, 0.04 mmol), Pd₂(dba)₃ (10 mg, 0.01 mmol), potassium pyridin-2-ylacetate (125 mg, 0.713 mmol, as prepared in the previous step), and mesitylene (1.2 mL) were added to a pressure tube that was pre-flushed with argon. It was sparged with more argon, sealed, and heated at 150° C. for 24 h. The mixture was cooled to rt and purified by silica gel flash chromatography (acetone with 0.1% Et₃N/DCM) to afford 72 mg (59%) of 2-chloro-5-(pyridin-2-ylmethyl)pyrimidine (Compound S80) as an oil. ¹H NMR (400 MHZ, CDCl₃) d (ppm) 8.59 (s, 2H) 8.57 (m, 1H) 7.68 (td, J=7.6, 1.8 Hz 1H) 7.21 (m, 2H) 4.13 (s, 2H); MS (ESI) m/z [M+H]⁺ calcd. for C₁₀H₉ClN₃: 206.5; found, 206.0; HPLC purity: 210 nm: 93.3%; 254 nm: 100.0%.

### Synthesis of 2-chloro-5-(pyridin-3-ylmethyl)pyrimidine (Compound S81)

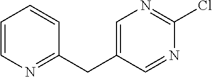 [SMILES: Clc1ncc(Cc2ccccn2)cn1]

3-(bromomethyl)pyridine hydrobromide (100 mg, 0.40 mmol), (2-chloropyrimidin-5-yl) boronic acid (94 mg, 0.59 mmol), Pd(dppf)Cl₂·DCM (32 mg, 0.04 mmol), (9:1) sat. aq. NaHCO₃ (1.9 mL, 1.98 mmol) and ACN (2.5 mL) were combined. The mixture was sparged with nitrogen for 20 min, and heated at 85° C. for 30 min. The mixture was cooled to rt and partitioned between EtOAc and more water. The organic layer was washed with water and brine, dried with Na₂SO₄, and filtered. The filtrate was concentrated under educed pressure then the residue was purified by silica gel flash chromatography (acetone with 0.1% Et₃N/hexane) to afford 6 mg (7%) of 2-chloro-5-(pyridin-3-ylmethyl)pyrimidine (Compound S81) as a solid. ¹H NMR (400 MHZ, CDCl₃) δ (ppm) 8.56 (d, J=3.5 Hz, 1H) 8.53 (br s, 1H) 8.48 (s, 2H) 7.47 (t, J=7.8 Hz 1H) 7.29 (m, 1H) 3.99 (s, 2H); MS (ESI) m/z [M+H]⁺ calcd. for C₁₀H₉ClN₃: 206.1; found, 206.0; HPLC purity: 210 nm: 91.1%; 254 nm: 91.1%.

### Synthesis of tert-butyl 4-(5-(1-(4-fluorophenyl)vinyl)pyrimidin-2-yl)piperazine-1-carboxylate (Compound S82)

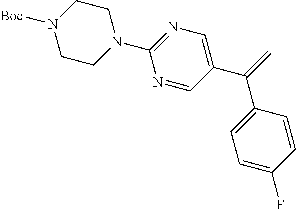 [SMILES: C=C(c1ccc(F)cc1)c1cnc(N2CCN(C(=O)OC(C)(C)C)CC2)nc1]

To an oven-dried 250 mL round bottom flask equipped with a magnetic stir bar was added triphenylmethylphosphonium bromide (1.67 g, 4.67 mmol) and THF (34 mL), then 1M NaHMDS in THF (5.12 mL, 5.12 mmol) was added dropwise and the mixture was stirred at rt for 45 min. A solution of tert-butyl 4-[5-(4-fluorobenzoyl)pyrimidin-2-yl]piperazine-1-carboxylate (0.860 g, 2.22 mmol, as prepared in Example 28, Step 4) in THF (26 mL) was added dropwise, then the reaction stirred at rt overnight. The reaction mixture was concentrated under reduced pressure then the residue was partitioned between DCM (50 mL) and H₂O (50 mL). The organic phase was washed with brine (20 mL), dried over anhydrous Na₂SO₄, and filtered, then the filtrate was concentrated under reduced pressure. The residue was purified by silica gel chromatography eluting with 10-20% EtOAc/hexanes to afford 692 mg (81%) of tert-butyl 4-(5-(1-(4-fluorophenyl)vinyl)pyrimidin-2-yl)piperazine-1-carboxylate (Compound S82) as a white solid. ¹H NMR (400 MHZ, CDCl₃) δ (ppm) 8.29 (s, 2H), 7.34-7.30 (m, 2H), 7.06-7.01 (m, 2H), 5.36 (s, 1H), 5.33 (s, 1H), 3.91-3.79 (m, 4H), 3.59-3.44 (m, 4H), 1.50 (s, 9H).

### Synthesis of 7-(1-methyl-1H-pyrazol-4-yl)-3-(piperazin-1-yl)imidazo[1,2-b]pyridazine hydrochloride salt (Compound S83)

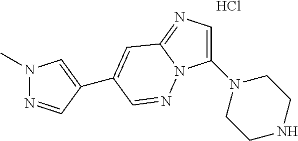 [SMILES: Cl ; Cn1cc(-c2cnn3c(N4CCNCC4)cnc3c2)cn1]

### Step 1. Synthesis of tert-butyl 4-(7-(1-methyl-1H-pyrazol-4-yl)imidazo[1,2-b]pyridazin-3-yl)piperazine-1-carboxylate

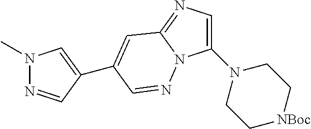 [SMILES: Cn1cc(-c2cnn3c(N4CCN(C(=O)OC(C)(C)C)CC4)cnc3c2)cn1]

A 20 mL vial containing 3-bromo-7-(1-methyl-1H-pyrazol-4-yl)imidazo[1,2-b]pyridazine (Compound S3) (300.4 mg, 1.080 mmol) and tert-butyl 1-piperazinecarboxylate (1.00 g, 5.40 mmol) was treated with tBuOH (3.0 mL) and dioxane (1.5 mL). The solution was sparged with argon for 10 min then KOtBu (182 mg, 1.62 mmol) was added, and the mixture was sparged again for 10 min with argon. tBuXPhos Pd G1 (111 mg, 0.162 mmol) was added, and the mixture was sparged for 10 min with argon. The mixture was stirred at 55° C. under an argon for 1 h. The reaction was cooled to rt, diluted with DCM (40 mL) and washed with 10% citric acid solution (3×10 mL), H₂O (3×10 mL), and sat. NaHCO₃ solution (2×10 mL). The organic layer was dried over anhydrous Na₂SO₄ and filtered, then the filtrate was concentrated under reduced pressure. The residue was purified by silica gel flash chromatography eluted with 0-100% (10% MeOH in DCM)/DCM affording 280.8 mg (68%) of tert-butyl 4-(7-(1-methyl-1H-pyrazol-4-yl)imidazo[1,2-b]pyridazin-3-yl)piperazine-1-carboxylate as a yellow solid. ¹H NMR (400 MHZ, CDCl₃) δ (ppm) 8.51 (d, J=2.0 Hz, 1H), 7.91 (d, J=2.0 Hz, 1H), 7.84 (s, 1H), 7.75 (s, 1H), 7.25 (s, 1H), 4.00 (s, 3H), 3.75-3.64 (m, 4H), 3.29-3.20 (m, 4H), 1.51 (s, 9H); MS (ESI) m/z [M+H]⁺ calcd. for C₁₉H₂₅N₇O₂: 384.2, Found: 384.2; HPLC purity: 210 nm: 97.8%; 254 nm: 100.0%.

### Step 2. Synthesis of 7-(1-methyl-1H-pyrazol-4-yl)-3-(piperazin-1-yl)imidazo[1,2-b]pyridazine hydrochloride Salt (Compound S83)

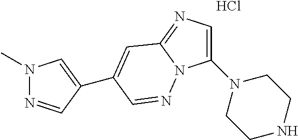 [SMILES: Cl ; Cn1cc(-c2cnn3c(N4CCNCC4)cnc3c2)cn1]

A mixture of tert-butyl 4-[7-(1-methyl-1H-pyrazol-4-yl)imidazo[1,2-b]pyridazin-3-yl]piperazine-1-carboxylate (274.5 mg, 0.7159 mmol, as prepared in the previous step) and DCM (30 mL) was cooled to 0° C. then a solution of 4 M HCl in dioxane (1.8 mL, 7.3 mmol) was added dropwise. After completion of addition, the mixture was warmed to rt and stirred for 7 h. The reaction was concentrated under reduced pressure, then the residue was triturated with ether and filtered. The solid was dried to afford 214.6 mg (94%) of 7-(1-methyl-1H-pyrazol-4-yl)-3-(piperazin-1-yl)imidazo[1,2-b]pyridazine hydrochloride salt (Compound S83) as a yellow solid. ¹H NMR (400 MHZ, DMSO-d₆) δ (ppm) 9.69 (br s, 2H), 9.34 (d, J=2.0 Hz, 1H), 8.69 (s, 1H), 8.44 (d, J=2.0 Hz, 1H), 8.34 (s, 1H), 7.98 (s, 1H), 3.94 (s, 3H), 3.54-3.49 (m, 4H), 3.37-3.25 (m, 4H); MS (ESI) m/z [M+H]⁺ calcd. for C₁₄H₁₇N₇: 284.2, Found: 284.2; HPLC purity: 210 nm: 95.9%; 254 nm: 100.0%.

### Synthesis of 1-(3-fluoro-4-(trifluoromethyl)phenyl)ethan-1-ol (Compound S84)

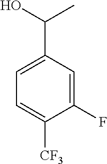

### Step 1. Preparation of 3-fluoro-N-methoxy-N-methyl-4-(trifluoromethyl)benzamide

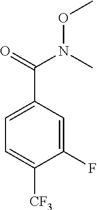 [SMILES: CON(C)C(=O)c1ccc(C(F)(F)F)c(F)c1]

To a 1 L round bottom flask equipped with a magnetic stir bar was added 3-fluoro-4-(trifluoromethyl)benzoic acid (10.0 g, 48.0 mmol) then DCM (500 mL) was added. The mixture was cooled to 0° C., and HOBt (9.513 g, 48.45 mmol), N,O-dimethylhydroxylamine hydrochloride (4.726 g, 48.45 mmol), and DIPEA (8.440 mL, 48.45 mmol) were added. EDCI (9.289 g, 48.45 mmol) was added and the mixture was stirred at 0° C. for 1 h, then warmed to rt and stirred for 7 h. Additional EDCl (1.472 g, 9.610 mmol), N,O-dimethylhydroxylamine hydrochloride (0.9374 g, 9.610 mmol), DIPEA (1.674 mL, 9.61 mmol), and EDCl (1.842 g, 9.610 mmol) were added and the mixture was stirred at rt overnight. The reaction was washed with sat. NaHCO₃ solution (120 mL), water (120 mL), and brine (2×120 mL), then the organic layer was dried over anhydrous Na₂SO₄ and filtered. The filtrate was concentrated under reduced pressure to afford 11.82 g (98%) of 3-fluoro-N-methoxy-N-methyl-4-(trifluoromethyl)benzamide as a brown oil. ¹H NMR (400 MHZ, CDCl₃) δ (ppm) 7.70-7.63 (m, 1H), 7.61-7.51 (m, 2H), 3.59-3.53 (m, 3H), 3.41-3.37 (m, 3H); MS (ESI) m/z [M+H]⁺ calcd. for C₁₀H₉F₄NO₂: 252.1, Found: 252.1; HPLC purity: 210 nm: 97.8%; 254 nm: 100.0%.

### Step 2. Preparation of 1-(3-fluoro-4-(trifluoromethyl)phenyl)ethan-1-one

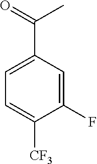

To a 250 mL round bottom flask equipped with a magnetic stir bar was added 3-fluoro-N-methoxy-N-methyl-4-(trifluoromethyl)benzamide (6.92 g, 27.6 mmol, as prepared in the previous step) and anhydrous THF (50 mL). The mixture was cooled to −10° C., then 3.4 M solution of MeMgBr in THF (20.2 mL, 68.9 mmol) was added dropwise over 40 min maintaining the low temperature. The reaction was stirred at −10° C. for 1 h, then quenched by the dropwise addition of sat. aq. NH₄Cl solution (70 mL). The mixture was warmed to rt, diluted with EtOAc and the phases were separated. The organic layer was washed with brine (70 mL), dried over anhydrous Na₂SO₄, and filtered. The filtrate was concentrated under reduced pressure, then the residue was purified by silica gel flash chromatography using eluted with 0-10% afford EtOAc/hexane to 4.28 g (75%) of 1-(3-fluoro-4-(trifluoromethyl)phenyl)ethan-1-one as a colorless oil. ¹H NMR (400 MHZ, CDCl₃) δ (ppm) 7.86-7.80 (m, 1H), 7.79-7.71 (m, 2H), 2.65 (s, 3H); MS (ESI) m/z [M+H]⁺ calcd. for C₉H₆F₄O: 207.2, Found: 207.2; HPLC purity: 210 nm: 100.0%; 254 nm: 100.0%.

### Step 3. Preparation of 1-(3-fluoro-4-(trifluoromethyl)phenyl)ethan-1-ol (Compound S84)

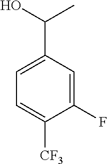

A solution of 1-(3-fluoro-4-(trifluoromethyl)phenyl)ethan-1-one (1.00 g, 4.85 mmol, as prepared in the previous step) in MeOH (10 mL) was cooled to 0° C. then NaBH₄ (0.184 g, 4.85 mmol) was added. The reaction was stirred at 0° C. for 1 h then the solvent was removed under reduced pressure. The residue was partitioned between EtOAc and water, then the aqueous layer was extracted with EtOAc (2×). The combined organic layers were washed with brine, dried over anhydrous Na₂SO₄, and filtered. The filtrate was concentrated under reduced pressure, then the residue was treated with hexanes and concentrated under reduced pressure to afford 960.8 mg (95%) of 1-(3-fluoro-4-(trifluoromethyl)phenyl)ethan-1-ol (Compound S84) as a colorless oil. ¹H NMR (400 MHZ, CDCl₃) δ (ppm) 7.58 (t, J=7.6 Hz, 1H), 7.30-7.19 (m, 2H), 4.96 (q, J=6.4 Hz, 1H), 1.83 (br s, 1H), 1.51 (d, J=6.4 Hz, 3H); HPLC purity: 210 nm: 95.9%; 254 nm: 100.0%.

### Synthesis of (4-chlorobenzyl)sulfamoyl fluoride (Compound S85)

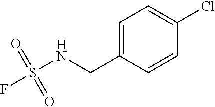 [SMILES: O=S(=O)(F)NCc1ccc(Cl)cc1]

To a 50 mL round bottom flask equipped with a magnetic stir bar and a rubber septum with a nitrogen inlet needle was added 4-chlorobenzylamine (194 mg, 1.37 mmol) and DCM (3.9 mL), then the solution was cooled in an ice bath. 1-(fluorosulfonyl)-2,3-dimethyl-1H-imidazol-3-ium trifluoromethanesulfonate (450 mg, 1.4 mmol) was added then the ice bath was removed. The reaction was stirred at rt for 2 h then the mixture was diluted with DCM, washed with 0.1N HCl (3×2 mL) and brine (1×3 mL). The clear organic layer was dried with anhydrous Na₂SO₄, filtered, and concentrated under reduced pressure to afford 0.250 g (82%) of (4-chlorobenzyl)sulfamoyl fluoride (Compound S85) as a colorless oil. ¹H NMR (400 MHZ, DMSO-d₆) δ (ppm) 9.95 (d, J=4.0 Hz, 1H) 7.45 (m, 2H) 7.37 (m, 2H) 4.36 (d, J=3.7 Hz, 2H).

Using the procedures described for Compound S85 and reagents, starting materials, and conditions known to those skilled in the art, the following compounds were prepared:

| Compound | Name | Characterization |
|---|---|---|
| Compound S86 | (R)-(1-(4- | ¹H NMR (400 MHz, |
| | chlorophenyl)ethyl)sulfamoyl | DMSO-d₆) δ (ppm) 9.97 |
| | fluoride | (br s, 1H), 7.46-7.39 (m, |
| | | 4H), 4.70 (p, J = 6.8 Hz, |
| | | 1H), 1.45 (d, J = 6.8 Hz, |
| | | 3H) |
| Compound S87 | (4- | ¹H NMR (400 MHz, |
| | (trifluoromethyl)benzyl)sulfamoyl | DMSO-d₆) δ (ppm) 10.06 |
| | fluoride | (br s, 1H), 7.77 (d, J = 8.0 |
| | | Hz, 2H), 7.58 (d, J = 8.0 |
| | | Hz, 2H), 4.48 (s, 2H) |

### Synthesis of 5-benzyl-2-chloropyrimidine (Compound S88)

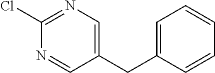 [SMILES: Clc1ncc(Cc2ccccc2)cn1]

To a mixture of (2-chloropyrimidin-5-yl) boronic acid (20 g, 0.126 mol) and Na₂CO₃ (40.43 g, 0.381 mol) in dioxane (200 mL) and H₂O (80 mL) stirred under nitrogen at 25° C. was added (Ph₃P₂PdCl₂ (4.43 g, 0.0063 mol); benzyl bromide (23.76 g, 0.138 mol) was added after 20 min The reaction mixture was stirred at 100° C. for 1 h. The mixture was diluted with water and extracted with EtOAc (3×50 mL). The combined organic layers were concentrated under vacuum to give crude product, which was purified by silica gel column eluting with DCM/MeOH (30:1˜15:1) to afford 18.18 g (69%) of 5-benzyl-2-chloropyrimidine (Compound S88) as a white solid. ¹H NMR (CDCl₃, 400 MHZ): δ (ppm) 8.48 (s, 2H), 7.34˜7.37 (m, 2H), 7.27˜7.31 (m, 1H), 7.19 (d, J=7.2 Hz, 2H), 3.98 (s, 2H).

### Synthesis of 4-(3-(4-iodo-1H-imidazol-1-yl)propyl)morpholine (Compound S89) and 4-[3-(5-iodoimidazol-1-yl)propyl]morpholine (Compound S90)

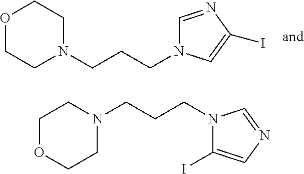

To a solution of 4-iodo-1H-imidazole (5 g, 25.78 mmol) and 4-(3-chloropropyl)morpholine (5.06 g, 30.93 mmol) in DMF (20 mL) was added Cs₂CO₃ (16.8 g, 51.55 mmol) at rt. The reaction was stirred for 2 h at 80° C. The resulting mixture was diluted with water (200 mL). The resulting mixture was extracted with EtOAc (3×200 mL). The combined organic layers were dried over anhydrous Na₂SO₄. After filtration, the filtrate was concentrated under reduced pressure. The residue was purified by reverse phase flash chromatography eluted with ACN: H₂O=2:8 to afford 2.7 g (32.6%) of 4-(3-(4-iodo-1H-imidazol-1-yl)propyl)morpholine (Compound S89) as a white solid and 900 mg (11%) of 4-[3-(5-iodoimidazol-1-yl)propyl]morpholine (Compound S90) as a white solid.

4-(3-(4-iodo-1H-imidazol-1-yl)propyl)morpholine (Compound S89): ¹H NMR (400 MHZ, DMSO-d₆) δ (ppm) 7.60 (d, J=1.4 Hz, 1H), 7.40 (d, J=1.4 Hz, 1H), 3.99 (t, J=7.0 Hz, 2H), 3.59 (t, J=4.6 Hz, 4H), 2.48-2.21 (m, 6H), 1.87-1.84 (m, 2H). MS (ESI) m/z [M+H]⁺ calcd. for C₁₀H₁₆IN₃O, 322.0; found, 322.0.

4-[3-(5-iodoimidazol-1-yl)propyl]morpholine (Compound S90): ¹H NMR (400 MHz, DMSO-d₆) δ (ppm) 7.85 (s, 1H), 7.00 (s, 1H), 3.97 (t, J=8.0 Hz, 2H), 3.64-3.56 (m, 4H), 2.50-2.21 (m, 6H), 1.88-1.83 (m, 2H). MS (ESI) m/z [M+H]⁺ calcd. for C₁₀H₁₆IN₃O, 322.0; found, 322.0.

### Synthesis of 4-iodo-1-(2-methoxyethyl)-1H-imidazole (Compound S91) and 5-iodo-1-(2-methoxyethyl)-1H-imidazole (Compound S92)

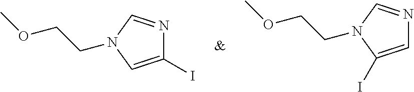

To a solution of 4-iodo-1H-imidazole (4 g, 20.62 mmol) and 1-bromo-2-methoxyethane (3.44 g, 24.74 mmol) in DMF (100 mL) were added Cs₂CO₃ (13.4 g, 41.24 mmol). After stirring for 16 h at 80° C. under a nitrogen atmosphere, the resulting mixture was filtered, the filter cake was washed with EtOAc (3×100 mL). The filtrate was concentrated under reduced pressure. The residue was purified by reversed-phase flash chromatography with a C18 silica gel column eluting with ACN in water (0.1% NH₃), 10% to 20% to afford 3 g (57.7%) of 4-iodo-1-(2-methoxyethyl)-1H-imidazole (Compound S91) as a light yellow solid and 2.1 g (40%) of 5-iodo-1-(2-methoxyethyl)-1H-imidazole (Compound S92) as a light yellow solid.

4-iodo-1-(2-methoxyethyl)-1H-imidazole (Compound S91): ¹H NMR (400 MHZ, DMSO-d₆) δ (ppm) 7.59 (d, J=1.4 Hz, 1H), 7.35 (d, J=1.3 Hz, 1H), 4.12 (t, J=5.2 Hz, 2H), 3.57 (t, J=5.2 Hz, 2H), 3.24 (d, J=0.8 Hz, 3H); MS (ESI) m/z [M+H]⁺ calcd. for C₆H₉IN₂O, 253.0; found, 253.1.

5-iodo-1-(2-methoxyethyl)-1H-imidazole (Compound S92): ¹H NMR (400 MHZ, DMSO-d₆) δ (ppm) 7.81 (s, 1H), 7.00 (s, 1H), 4.09 (t, J=5.4 Hz, 2H), 3.58 (t, J=5.4 Hz, 2H), 3.24 (s, 3H); MS (ESI) m/z [M+H]⁺ calcd. for C₆H₉IN₂O, 253.0; found, 253.1.

### Synthesis of 1-(2-((tert-butyldimethylsilyl)oxy)ethyl)-4-iodo-1H-imidazole (Compound S93) and 1-(2-((tert-butyldimethylsilyl)oxy)ethyl)-5-iodo-1H-imidazole (Compound S94)

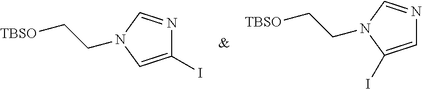 [SMILES: CC(C)(C)[Si](C)(C)OCCn1cnc(I)c1 ; CC(C)(C)[Si](C)(C)OCCn1cncc1I]

To a solution of 4-iodo-1H-imidazole (2.0 g, 10.31 mmol) and tert-butyl (2-iodoethoxy)dimethylsilane (4.43 g, 15.47 mmol) in DMF (150 mL) was added Cs₂CO₃ (6.72 g, 20.62 mmol) in portions at rt under nitrogen atmosphere. The final reaction mixture was stirred for overnight at 80° C. Then mixture was allowed to cool down to rt. The resulting mixture was concentrated under reduced pressure. The residue was purified by silica gel column chromatography, eluted with PE/EtOAc (10:1) to afford 3.5 g (77%) of a mixture of 1-(2-((tert-butyldimethylsilyl)oxy)ethyl)-4-iodo-1H-imidazole (Compound S93) and 1-(2-((tert-butyldimethylsilyl)oxy)ethyl)-5-iodo-1H-imidazole (Compound S94) as a yellow oil. MS (ESI) m/z [M+H]⁺ calcd. for C₁₁H₂₁IN₂OSi, 353.2; found, 353.2.

### Synthesis of (R)-1-(4-chlorophenyl)ethyl (2,5-dioxopyrrolidin-1-yl)carbonate (Compound S95)

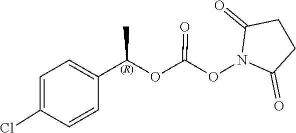

To a solution of (1R)-1-(4-chlorophenyl)ethanol (20.00 g, 127.7 mmol) in ACN (240 mL was added di(N-succinimidyl)carbonate (49.07 g, 191.6 mmol) then the mixture was cooled to 0° C. NEt₃ (53 mL, 380 mmol) was added dropwise over 5 min then stirred at 0° C. for 30 min. The solution allowed to warm to rt. The solution was stirred overnight at rt, then the mixture was concentrated under reduced pressure. The residue was diluted with EtOAc (250 mL) and washed with sat. NaHCO₃ solution. Additional EtOAc (100 mL) was added, then the organic layer was washed with water and brine, then dried over anhydrous Na₂SO₄, and filtered. The filtrate was concentrated under reduced pressure. To the residue was added MTBE (150 mL) and the mixture was heated to 45° C. Hexanes (100 mL) was added hot then the mixture was allowed to cool with efficient stirring. The solids were isolated by filtration, washed with hexanes (100 mL), and dried under reduced pressure to afford 31.85 g (84%) of (R)-1-(4-chlorophenyl)ethyl (2,5-dioxopyrrolidin-1-yl)carbonate (Compound S95) as a light tan solid. ¹H NMR (400 MHZ, DMSO-d₆) δ (ppm) 7.49 (s, 4H), 5.89 (q, J=6.8 Hz, 1H), 2.80 (s, 4H), 1.62 (d, J=6.8 Hz, 3H).

### Synthesis of 2-(chloromethyl)-5-methyloxazole (Compound S96)

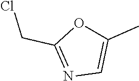

### Step 1. Synthesis of (5-methyloxazol-2-yl)methanol

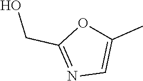

To a solution of ethyl 5-methyl-1,3-oxazole-2-carboxylate (500 mg, 3.22 mmol) in EtOH (10 mL) was added NaBH₄ (183 mg, 4.84 mmol). The resulting mixture was stirred for 2 h at rt. The reaction was quenched with 30 mL sat. aq. NaHCO₃ at 0° C. The resulting mixture was concentrated under reduced pressure. The resulting mixture was extracted with DCM/MeOH (9:1) (5×50 mL). The combined organic layers were dried over anhydrous Na₂SO₄. After filtration, the filtrate was concentrated under reduced pressure to afford 320 mg (79%) of (5-methyloxazol-2-yl)methanol as a yellow liquid. ¹H NMR (400 MHZ, DMSO-d₆) δ (ppm) 6.76 (d, J=1.4 Hz, 1H), 5.57 (t, J=6.1 Hz, 1H), 4.42 (d, J=6.1 Hz, 2H), 2.28 (d, J=1.3 Hz, 3H); MS (ESI) m/z [M+H]⁺ calcd. for C₅H₇NO₂, 114.0; found, 114.1.

### Step 2. Preparation of 2-(chloromethyl)-5-methyloxazole (Compound S96)

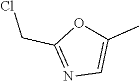

To a solution of (5-methyloxazol-2-yl)methanol (100 mg, 0.88 mmol, as prepared in the previous step) and NEt₃ (134 mg, 1.33 mmol) in DCM (6 mL) was added SOCl₂ (210 mg, 1.77 mmol) at 0° C. The resulting mixture was stirred for 1 h at 0° C. The resulting mixture was concentrated under reduced pressure to afford 100 mg (86%) of 2-(chloromethyl)-5-methyloxazole (Compound S96) as a yellow solid. MS (ESI) m/z [M+H]⁺ calcd. for C₅H₆ClNO, 132.0; found, 132.1.

### Synthesis of 6-(1-methyl-1H-pyrazol-4-yl)pyrimidin-4-amine (Compound S97)

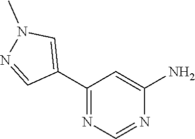

To a mixture of 6-chloropyrimidin-4-amine (13.4 g, 103.5 mmol) in dioxane (260 mL) and H₂O (50 mL) was added 1-methyl-4-(4,4,5,5-tetramethyl-1,3,2-dioxaborolan-2-yl)-1H-pyrazole (32.3 g, 155.2 mmol), Na₂CO₃ (24 g, 227.6 mmol), and Pd(dppf)Cl₂ (3.3 g) under nitrogen. The mixture was heated to reflux and stirred under nitrogen for 6 h. The mixture was cooled and poured into cold water, extracted with DCM/MeOH=10/1 ten times, the organic phase was washed with brine, dried over anhydrous Na₂SO₄, and filtered. The filtrate was concentrated under reduced pressure then the residue was purified by silica gel chromatography to afford 8.7 g (48%) of 6-(1-methyl-1H-pyrazol-4-yl)pyrimidin-4-amine (Compound S97) as yellow solid. ¹H NMR (400 MHZ, DMSO-d₆) δ (ppm) 8.29 (s, 1H), 8.22 (s, 1H), 7.88 (s, 1H), 6.74 (s, 2H), 6.54 (s, 1H), 3.87 (s, 3H).

### Synthesis of 6-(1-Methyl-1H-pyrazol-4-yl)-3-piperazin-1-ylpyrazolo[1,5-a]pyrimidine (Compound S98)

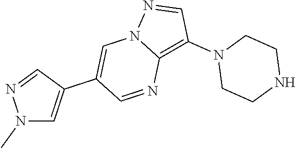 [SMILES: Cn1cc(-c2cnc3c(N4CCNCC4)cnn3c2)cn1]

### Step 1. Preparation of tert-butyl 4-(6-(1-methyl-1H-pyrazol-4-yl)pyrazolo[1,5-a]pyrimidin-3-yl)piperazine-1-carboxylate

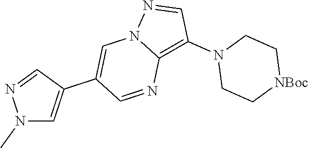 [SMILES: Cn1cc(-c2cnc3c(N4CCN(C(=O)OC(C)(C)C)CC4)cnn3c2)cn1]

To a vial was added 3-bromo-6-(1-methyl-1H-pyrazol-4-yl)pyrazolo[1,5-a]pyrimidine (0.150 g, 0.539 mmol) and tert-butyl 1-piperazinecarboxylate (0.120 g, 0.647 mmol) followed by tBuOH (1.5 mL, 16 mmol) and dioxane (3 mL, 40 mmol). The mixture was sparged with argon for 5 min then NaOtBu (0.0778 g, 0.809 mmol) was added, and the mixture was sparged for 5 min with argon. To the mixture was added tBuXPhos Pd G1 (37.0 mg, 0.0539 mmol) and the mixture was sparged with argon for 5 min. The reaction was heated to 80° C. for 2.5 h then additional tBuXPhos Pd G1 (15 mg) was added as a slurry in dioxane (0.5 mL) and the mixture was stirred at 80° C. overnight. The reaction was cooled to rt, quenched with sat. NaHCO₃ and extracted with DCM (3×). The combined organic layer was washed with water, dried over anhydrous Na₂SO₄, and filtered. The residue was purified by silica gel chromatography to afford 95 mg (46%) of tert-butyl 4-(6-(1-methyl-1H-pyrazol-4-yl)pyrazolo[1,5-a]pyrimidin-3-yl)piperazine-1-carboxylate as a yellow-orange solid. ¹H NMR (400 MHZ, CDCl₃) δ (ppm) 8.58 (d, J=2.0 Hz, 1H), 8.45 (d, J=2.4 Hz, 1H), 7.78 (s, 1H), 7.77 (s, 1H), 7.68 (s, 1H), 4.01 (s, 3H), 3.71-3.65 (m, 4H), 3.30-3.24 (m, 4H), 1.51 (s, 9H). MS (ESI) m/z [M+H]⁺ calcd. for C₁₉H₂₅N₇O₂, 384.2; found, 384.3.

### Step 2. Preparation of 6-(1-Methyl-1H-pyrazol-4-yl)-3-piperazin-1-ylpyrazolo[1,5-a]pyrimidine (Compound S98)

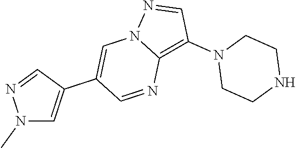 [SMILES: Cn1cc(-c2cnc3c(N4CCNCC4)cnn3c2)cn1]

To a solution of tert-butyl 4-[6-(1-methyl-1H-pyrazol-4-yl)pyrazolo[1,5-a]pyrimidin-3-yl]piperazine-1-carboxylate (0.09 g, 0.2 mmol, as prepared in the previous step) in DCM (2 mL) was added TFA (0.2 mL, 2 mmol) and the reaction was stirred at rt overnight. The mixture was diluted with DCM (20 mL) and washed with sat. aq. NaHCO₃ (25 mL). The aqueous layer was extracted with DCM (2×20 mL) and the combined organic fractions were dried over anhydrous Na₂SO₄, and filtered. The filtrate was concentrated under reduced pressure to afford 0.065 g (93%) of 6-(1-methyl-1H-pyrazol-4-yl)-3-piperazin-1-ylpyrazolo[1,5-a]pyrimidine (Compound S98) as an orange solid. ¹H NMR (400 MHZ, CDCl₃) δ (ppm) 8.58 (d, J=2.0 Hz, 1H), 8.45 (d, J=2.4 Hz, 1H), 7.79 (s, 1H), 7.77 (s, 1H), 7.68 (s, 1H), 4.01 (s, 3H), 3.37-3.31 (m, 4H), 3.20-3.14 (m, 4H). MS (ESI) m/z [M+H]⁺ calcd. for C₁₄H₁₇N₇, 284.2; found, 284.1.

### Synthesis of 4-(1-methyl-1H-pyrazol-4-yl)pyrimidin-2-amine (Compound S99)

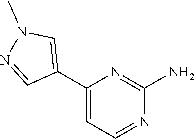

To a mixture of 4-chloropyrimidin-2-amine (20 g, 154.4 mmol) in dioxane/water (8/1, 480 mL) was added 1-methyl-4-(4,4,5,5-tetramethyl-1,3,2-dioxaborolan-2-yl)-1H-pyrazole (48.2 g, 231.6 mmol), Na₂CO₃ (36.0 g, 339.6 mmol) and Pd(dppf)Cl₂ (3.0 g) under nitrogen. The mixture was heated to reflux and stirred under nitrogen for 2 h. The mixture was cooled to rt, filtered. The filtrate was poured into cold water, extracted with DCM/MeOH=10/1 ten times, the organic phase was washed with brine, dried over anhydrous Na₂SO₄, and filtered. The filtrate was concentrated under reduced pressure. The residue was purified by silica gel chromatography eluting with DCM/MeOH (1:0˜50:1) to afford 20 g (74%) of 4-(1-methyl-1H-pyrazol-4-yl)pyrimidin-2-amine (Compound S99) as a yellow solid. ¹H NMR (400 MHZ, DMSO-d₆) δ (ppm) 8.26 (s, 1H), 8.15 (d, J=4.8 Hz, 1H), 7.97 (s, 1H), 6.80 (d, J=5.2 Hz, 1H), 6.47 (s, 2H), 3.88 (s, 3H).

### Example 4: Synthesis of Exemplary Compound 1

### 6-(1-methyl-1H-pyrazol-4-yl)-3-[4-(3-phenylpropanoyl)piperazin-1-yl]pyrazolo[1,5-a]pyridine (Compound 1)

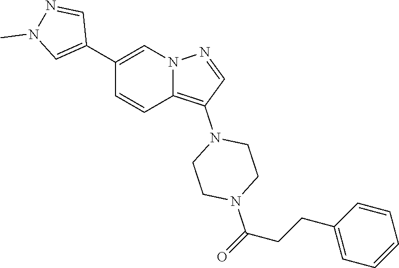 [SMILES: Cn1cc(-c2ccc3c(N4CCN(C(=O)CCc5ccccc5)CC4)cnn3c2)cn1]

A vial containing 6-(1-methyl-1H-pyrazol-4-yl)-3-piperazin-1-ylpyrazolo[1,5-a]pyridine dihydrochloride (Compound S19, 50 mg, 0.16 mmol) suspended in DCM (0.50 mL) was cooled in an ice bath to 0° C. To the cold suspension was added Et₃N (48 mg, 0.47 mmol) followed by a solution of benzenepropanoyl chloride (26 mg, 0.16 mmol) in DCM (0.50 mL). The ice bath was removed, and the reaction stirred for 1 h. To the reaction was added H₂O (4 mL). The resulting two layers were separated. The aqueous layer was extracted three times with DCM (3×4 mL). The combined DCM layers were dried (Na₂SO₄), filtered and concentrated. The residue was purified by ISCO chromatography (4 g normal phase cartridge) eluting with a gradient of 0-10% IPA in EtOAc. The solids obtained were triturated with Et₂O and concentrated in vacuo. This was repeated three more times. The resulting solid was dried in vacuo to afford 33 mg (51%) of 6-(1-methyl-1H-pyrazol-4-yl)-3-[4-(3-phenylpropanoyl)piperazin-1-yl]pyrazolo[1,5-a]pyridine (Compound 1) as a grey solid. ¹H NMR (400 MHZ, CDCl₃): δ (ppm) 8.46 (s, 1H) 7.74 (s, 1H) 7.67 (s, 1H) 7.61 (s, 1H) 7.49 (d, J=9.17 Hz, 1H) 7.20-7.36 (m, 5H) 7.15 (dd, J=9.17, 1.47 Hz, 1H) 3.98 (s, 3H) 3.78-3.88 (m, 2H) 3.54-3.63 (m, 2H) 2.96-3.07 (m, 4H) 2.85-2.93 (m, 2H) 2.66-2.74 (m, 2H); MS (ESI) m/z [M+H]⁺ calcd. for C₂₄H₂₇N₆O: 415.2. Found: 415.3; HPLC purity: 210 nm: 100.0%; 254 nm: 100.0%.

### Example 5: Synthesis of Exemplary Compound 2

### tert-butyl 4-[6-(1-methyl-1H-pyrazol-4-yl)pyrazolo[1,5-a]pyrimidin-3-yl]piperazine-1-carboxylate (Compound 2)

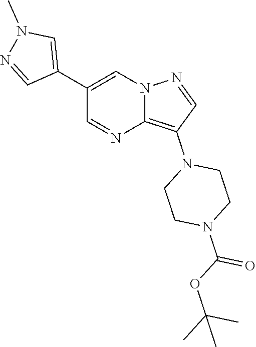 [SMILES: Cn1cc(-c2cnc3c(N4CCN(C(=O)OC(C)(C)C)CC4)cnn3c2)cn1]

A flask containing 3-bromo-6-(1-methyl-1H-pyrazol-4-yl)pyrazolo[1,5-a]pyrimidine (Compound S10, 0.150 g, 0.539 mmol) and tert-butyl 1-piperazinecarboxylate (0.120 g, 0.647 mmol) was treated with tert-butyl alcohol (1.96 mL, 20.5 mmol) and 1,4-dioxane (0.505 mL, 6.47 mmol). The solution was sparged with argon gas for 15 min. The solution was treated with tBuXPhos Pd G1 (0.056 g, 0.08 mmol) and sodium tert-butoxide (0.062 g, 0.65 mmol) and the mixture was sparged again for 10 min with argon gas. The reaction mixture was stirred under an atmosphere of argon in a 55° C. heating block for 12 h, followed by heating at 75° C. for 12 h. The catalyst was removed by filtration through Celite®-545 and the filter cake was washed with 20 mL of CH₂Cl₂. The filtrate was extracted with 2×20 mL of saturated NaHCO₃ and 20 mL of brine. Drying over Na₂SO₄ and evaporation gave 0.26 g of a brown solid. Chromatography on a Biotage Isolera® in 0% EtOAc/hexane (1 CV) to 100% EtOAc (over 15 CV) on 12 g of silica gel gave 25 mg of tert-butyl 4-[6-(1-methyl-1H-pyrazol-4-yl)pyrazolo[1,5-a]pyrimidin-3-yl]piperazine-1-carboxylate which was 93% pure by HPLC. The material was chromatographed on a Biotage Isolera® in hexane (3 CV) followed by 100% EtOAc (15 CV) on 4 g of silica gel to give 16.3 mg (8%) of tert-butyl 4-[6-(1-methyl-1H-pyrazol-4-yl)pyrazolo[1,5-a]pyrimidin-3-yl]piperazine-1-carboxylate (Compound 2) as an orange solid. ¹H NMR (CDCl₃, 400 MHZ): δ (ppm) 8.58 (d, J=2.1 Hz, 1H), 8.46 (d, J=2.1 Hz, 1H), 7.78 (d, J=2.1 Hz, 2H), 7.68 (s, 1H), 4.01 (s, 3H), 3.60-3.75 (m, 4H), 3.16-3.34 (m, 4H), 1.51 (s, 9H); MS (ESI) m/z [M+H]⁺ calcd. for C₁₉H₂₆N₇O₂: 384.4. Found: 384.3; HPLC purity: 201 nm: 96.7%; 254 nm: 98.5%.

### Example 6: Synthesis of Exemplary Compound 3

### 2,2-dimethylpropyl 4-[6-(1-methyl-1H-pyrazol-4-yl)pyrazolo[1,5-a]pyridin-3-yl]piperazine-1-carboxylate (Compound 3)

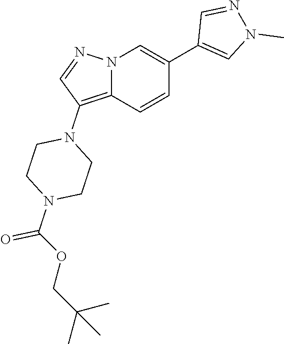 [SMILES: Cn1cc(-c2ccc3c(N4CCN(C(=O)OCC(C)(C)C)CC4)cnn3c2)cn1]

To a glass vial containing 6-(1-methyl-1H-pyrazol-4-yl)-3-piperazin-1-ylpyrazolo[1,5-a]pyridine dihydrochloride (Compound $19, 76 mg, 0.210 mmol) cooled at 4° C. was added DCM (1.5 mL) and triethylamine (149 μL, 1.07 mmol). 2,2-dimethylpropyl chlorocarbonate (39 μL, 0.26 mmol) was added dropwise and the mixture warmed to ambient temperature over 18 hours. The reaction was monitored by HPLC, and if necessary, was dosed with additional 2,2-dimethylpropyl chlorocarbonate until all 6-(1-methyl-1H-pyrazol-4-yl)-3-piperazin-1-ylpyrazolo[1,5-a]pyridine was consumed. The reaction mixture was diluted with dichloromethane and was purified by Biotage chromatography (4 g normal phase cartridge) eluting with a gradient of 0-90% acetone in DCM to afford 61 mg (66%) of 2,2′-dimethylpropyl 4-[6-(1-methyl-1H-pyrazol-4-yl)pyrazolo[1,5-a]pyridin-3-yl]piperazine-1-carboxylate (Compound 3) as a solid. ¹H NMR (400 MHZ, CDCl₃): δ (ppm) 8.48 (s, 1H), 7.76 (s, 1H), 7.73 (s, 1H), 7.63 (s, 1H), 7.52 (d, J=9.05 Hz, 1H), 7.15 (dd, J=9.11 Hz, 1.28 Hz, 1H), 3.99 (s, 3H), 3.85 (s, 2H), 3.73 (m, 4H), 3.08 (m, 4H), 0.99 (s, 9H); MS (ESI) m/z [M+H]⁺ calcd. for C₂₁H₂₉N₆O₂: 397.2. Found: 397.3; HPLC purity: 210 nm: 100%; 254 nm: 100%.

### Example 7: Synthesis of Exemplary Compound 4

### 3-[4-(5-benzylpyrimidin-2-yl)piperazin-1-yl]-5-(1-methyl-1H-pyrazol-4-yl)pyrazolo[1,5a]pyridine (Compound 4)

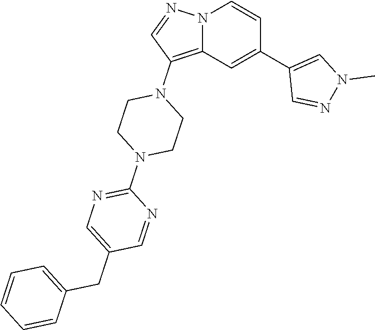 [SMILES: Cn1cc(-c2ccn3ncc(N4CCN(c5ncc(Cc6ccccc6)cn5)CC4)c3c2)cn1]

To a solution of 3-bromo-5-(1-methyl-1H-pyrazol-4-yl)pyrazolo[1,5-a]pyridine (Compound S11, 300 mg, 1.08 mmol) in t-butanol (8.0 mL) and 1,4-dioxane (2.0 mL) was added 5-benzyl-2-piperazin-1-ylpyrimidine (Compound S16, 330 mg, 1.30 mmol) and the solution was sparged with argon gas for 20 minutes. The solution was treated with tBuXPhos Pd G1 (37 mg, 0.054 mmol) and sodium t-butoxide (125 mg, 1.30 mmol) and the solution was sparged for 15 min with argon gas, followed by heating the reaction mixture at 55° C. under an argon atmosphere. After 16 h, HPLC indicated the reaction was complete. The reaction mixture was cooled to room temperature, diluted with 50 mL CH₂Cl₂ and washed twice with 50 mL portions of H₂O and once with 50 mL brine and dried over Na₂SO₄. The solution was filtered and concentrated to an orange viscous oil that was purified by flash chromatography (100 g silica gel; 20-40% acetone/CH₂Cl₂) resulting in a yellow glass that was treated with CH₃CN to obtain a solid. The solid was filtered to afford 99 mg (20%) of 3-[4-(5-benzylpyrimidin-2-yl)piperazin-1-yl]-5-(1-methyl-1H-pyrazol-4-yl)pyrazolo[1,5-a]pyridine (Compound 4) as a light yellow solid. ¹H NMR (400 MHZ, CDCl₃): δ (ppm) 8.28-8.36 (m, 1H) 8.22 (s, 2H) 7.83 (s, 1H) 7.75 (s, 1H) 7.71 (s, 1H) 7.55 (d, J=0.98 Hz, 1H) 7.28-7.36 (m, 2H) 7.15-7.26 (m, 3H) 6.82 (dd, J=7.21, 1.83 Hz, 1H) 4.00-4.06 (m, 4H) 3.98 (s, 3H) 3.82 (s, 2H) 3.07-3.19 (m, 4H); MS (ESI) m/z [M+H]⁺ calcd. for C₂₆H₂₇N₈, 451.2; found, 451.2; HPLC purity: 210 nm: 98.5%; 254 nm 99.4%.

### Example 8: Synthesis of Exemplary Compound 5

### 3-[4-(5-benzylpyrimidin-2-yl)piperazin-1-yl]-6-(1-methyl-1H-pyrazol-4-yl)[1,2,4]triazolo[4,3-a]pyridine (Compound 5)

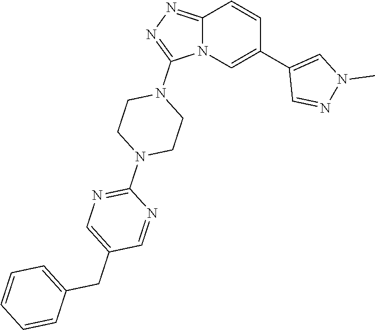 [SMILES: Cn1cc(-c2ccc3nnc(N4CCN(c5ncc(Cc6ccccc6)cn5)CC4)n3c2)cn1]

### Step 1. Preparation of tert-butyl 4-[6-(1-methyl-1H-pyrazol-4-yl)[1,2,4]triazolo[4, 3-a]pyridin-3-yl]piperazine-1-carboxylate

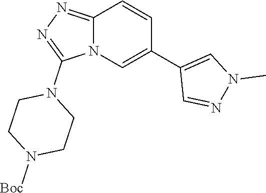 [SMILES: Cn1cc(-c2ccc3nnc(N4CCN(C(=O)OC(C)(C)C)CC4)n3c2)cn1]

To a solution of 3-chloro-6-(1-methyl-1H-pyrazol-4-yl)[1,2,4]triazolo[4,3-a]pyridine (Compound S12, 0.600 g, 2.57 mmol) in NMP (9.0 mL) was added piperazine (0.442 g, 5.14 mmol) and triethylenediamine (0.317 g, 2.82 mmol). The solution was heated at 160° C. overnight at which point the conversion was deemed complete by HPLC. The solution was cooled to rt and di-tert-butyldicarbonate (1.68 g, 7.70 mmol) was added. The solution was stirred for 2 h then diluted with EtOAc and washed with water and brine, dried (Na₂SO₄) filtered and concentrated. The residue was purified by Biotage chromatography (80 g cartridge) eluting with a gradient of EtOAc (containing 0.1% TEA) in hexanes followed by a gradient of 20% EtOH/EtOAc (containing 0.05% TEA) in EtOAc (containing 0.1% TEA) to afford 0.352 g (36%) of (tert-butyl 4-[6-(1-methyl-1H-pyrazol-4-yl)[1,2,4]triazolo[4,3-a]pyridin-3-yl]piperazine-1-carboxylate as a tan solid. MS (ESI) m/z [M+H]⁺ calcd. for C₁₉H₂₅N₇O₂, 384.2; found, 384.2.

### Step 2. Preparation of 3-[4-(5-benzylpyrimidin-2-yl)piperazin-1-yl]-6-(1-methyl-1H-pyrazol-4-yl)[1,2,4]triazolo[4,3-a]pyridine (Compound 5)

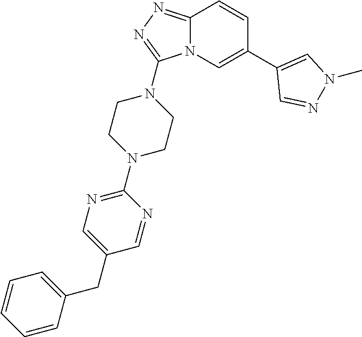 [SMILES: Cn1cc(-c2ccc3nnc(N4CCN(c5ncc(Cc6ccccc6)cn5)CC4)n3c2)cn1]

To a cooled (0-5° C.) solution of tert-butyl 4-[6-(1-methyl-1H-pyrazol-4-yl)[1,2,4]triazolo[4,3-a]pyridin-3-yl]piperazine-1-carboxylate (0.350 g, 0.91 mmol) in DCM (6 mL) was added trifluoroacetic acid (3 mL, 40 mmol). After 1 h, the volatiles were removed under reduced pressure at ambient temperature. The residue was concentrated twice from DCM and diethyl ether and dried in vacuo. The crude TFA salt was used in the next step without purification.

To a solution of the TFA salt in NMP (5 mL) was added K₂CO₃ (0.315 g, 2.28 mmol) and 5-benzyl-2-chloropyrimidine (0.243 g, 1.19 mmol). The mixture was heated to 110° C. for 3 h under an atmosphere of nitrogen. The mixture was cooled, diluted with DCM and the organics washed with satd aq. NaHCO₃ and water, dried (Na₂SO₄), filtered and concentrated. The residue was purified by Biotage chromatography (40 g normal phase cartridge) eluting with a gradient of ethyl acetate in hexanes followed by a second chromatography (20 g normal phase cartridge) using a gradient of ethanol (0-10% containing 0.1% TEA) in ethyl acetate to afford 155 mg (38%) of 3-[4-(5-benzylpyrimidin-2-yl)piperazin-1-yl]-6-(1-methyl-1H-pyrazol-4-yl)[1,2,4]triazolo[4,3-a]pyridine (Compound 5) as a cream colored solid. ¹H NMR (400 MHZ, CDCl₃): δ (ppm) 8.96 (s, 1H) 8.22 (s, 2H) 7.59 (d, J=9.29 Hz, 1H) 7.55 (s, 1H) 7.47 (s, 1H) 7.33 (t, J=7.40 Hz, 2H) 7.24 (t, J=7.34 Hz, 1H) 7.19 (d, J=9.17 Hz, 3H) 4.55 (br. s., 2H) 3.99 (s, 3H) 3.84 (s, 2H) 3.39 (br. s., 2H) 3.23 (d, J=5.38 Hz, 2H) 2.98 (br. s., 2H); MS (ESI) m/z [M+H]⁺ calcd. for C₂₆H₂₇N₈, 451.2; found, 451.2; HPLC purity: 210 nm: 100.0%; 254 nm: 100.0%.

### Example 9: Synthesis of Exemplary Compound 6

### 3-[4-(5-benzylpyrimidin-2-yl)piperazin-1-yl]-6-(1-methyl-1H-pyrazol-4-yl)pyrazolo[1,5-a]pyridine (Compound 6)

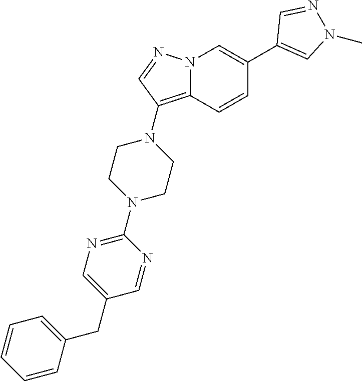 [SMILES: Cn1cc(-c2ccc3c(N4CCN(c5ncc(Cc6ccccc6)cn5)CC4)cnn3c2)cn1]

To a vial containing 3-bromo-6-(1-methyl-1H-pyrazol-4-yl)pyrazolo[1,5-a]pyridine (Compound S9, 1.00 g, 3.61 mmol) and 5-benzyl-2-piperazin-1-ylpyrimidine (Compound S16, 1.10 g, 4.33 mmol) was added tert-butyl alcohol (40 mL) and 1,4-dioxane (10 mL). The partially dissolved mixture was sparged with argon for 5 min. To the mixture was added sodium tert-butoxide (520 mg, 0.54 mmol). The mixture was sparged again for 5 min with argon and tBuXPhos Pd G1 (248 mg, 5.41 mmol) added. The reaction mixture was sparged with argon for 5 min and an additional 2 min with sonication. The flask was heated in a 55° C. oil bath for 2.5 h, cooled to ambient temperature and diluted with ethyl acetate. The mixture was washed with satd aq NaHCO₃, brine, dried (Na₂SO₄) filtered and concentrated. The residue was purified by Biotage chromatography (120 g normal phase cartridge) eluting with a gradient of 50-100% DCM in hexanes followed by a gradient of 0-20% acetone in DCM. The solids obtained were triturated with MeCN, collected by filtration, washed with MeCN and dried in vacuo to afford 0.76 g, (47%) of 3-[4-(5-benzylpyrimidin-2-yl)piperazin-1-yl]-6-(1-methyl-1H-pyrazol-4-yl)pyrazolo[1,5-a]pyridine (Compound 6) as a yellow solid. ¹H NMR (400 MHZ, CDCl₃): δ (ppm) 8.48 (s, 1H), 8.23 (s, 2H), 7.76 (s, 1H), 7.73 (s, 1H), 7.63 (s, 1H), 7.57 (d, J=9.17 Hz, 1H), 7.34-7.28 (m, 2H), 7.25-7.15 (m, 4H), 4.03-4.01 (m, 4H), 3.99 (s, 3H), 3.83 (s, 2H), 3.15-3.13 (m, 4H); MS (ESI) m/z [M+H]⁺ calcd. for C₂₆H₂₇N₈, 451.2; found, 451.3; HPLC purity: 210 nm: 100.0%; 254 nm: 100.0%.

### Example 10: Synthesis of Exemplary Compound 7

### 3-[4-(5-benzylpyrimidin-2-yl)piperazin-1-yl]-6-(1-methyl-1H-pyrazol-4-yl)pyrazolo[1,5-a]pyrimidine (Compound 7)

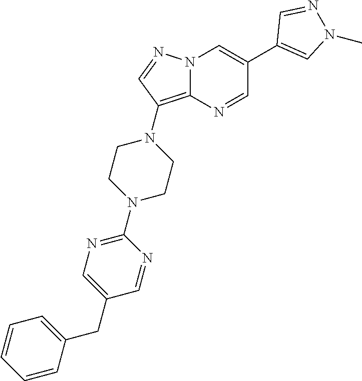 [SMILES: Cn1cc(-c2cnc3c(N4CCN(c5ncc(Cc6ccccc6)cn5)CC4)cnn3c2)cn1]

To a solution of 3-bromo-6-(1-methyl-1H-pyrazol-4-yl)pyrazolo[1,5-a]pyrimidine (Compound S10, 1.00 g, 3.60 mmol) in t-butanol (26.7 mL) and 1,4-dioxane (7.2 mL) was added 5-benzyl-2-piperazin-1-ylpyrimidine (Compound S16, 1.10 g, 4.31 mmol) and the mixture was sparged with argon gas for 20 min. The mixture was further treated with tBuXPhos Pd G1 (370 mg, 0.54 mmol) and sodium t-butoxide (0.52 g, 5.39 mmol) and the mixture was sparged for an additional 15 min with argon gas. The now brown slurry was heated at 55° C. for 4 h at which point it was determined by HPLC that the starting material had been consumed. The reaction mixture was cooled, diluted with 150 mL EtOAc and the solution was washed with two 100 mL portions of H₂O. The organic phase was dried over MgSO₄, filtered, and concentrated to a sticky brown solid. The crude product was purified by flash chromatography (200 g silica gel; 10-50% acetone/CH₂Cl₂) to yield a solid that was triturated with ethyl ether. The solid was filtered to afford 374 mg (23%) of 3-[4-(5-benzylpyrimidin-2-yl)piperazin-1-yl]-6-(1-methyl-1H-pyrazol-4-yl)pyrazolo[1,5-a]pyrimidine as an orange solid. ¹H NMR (400 MHz, CDCl₃): δ (ppm) 8.57 (d, J=2.08 Hz, 1H) 8.46 (d, J=2.08 Hz, 1H) 8.21 (s, 2H) 7.80 (s, 1H) 7.76 (s, 1H) 7.67 (s, 1H) 7.28-7.34 (m, 2H) 7.14-7.25 (m, 3H) 4.02-4.08 (m, 4H) 4.00 (s, 3H) 3.81 (s, 2H) 3.31-3.44 (m, 4H); MS (ESI) m/z [M+H]⁺ calcd. for C₂₅H₂₆N₉, 452.2; found, 452.3; HPLC purity: 210 nm: 96.9%; 254 nm 98.7%.

### Example 11: Synthesis of Exemplary Compound 8

### 3-[4-(5-benzylpyrimidin-2-yl)piperazin-1-yl]-6-(1-methyl-1H-pyrazol-4-yl)pyrazolo[1,5-b]pyridazine (Compound 8)

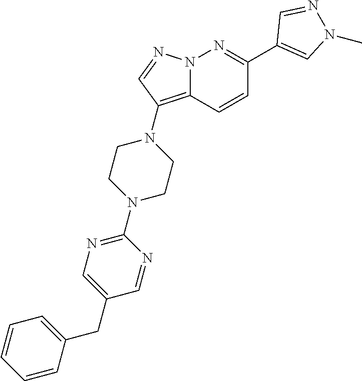 [SMILES: Cn1cc(-c2ccc3c(N4CCN(c5ncc(Cc6ccccc6)cn5)CC4)cnn3n2)cn1]

A mixture of 3-bromo-6-(1-methyl-1H-pyrazol-4-yl)pyrazolo[1,5-b]pyridazine (Compound S8, 0.500 g, 1.80 mmol), 5-benzyl-2-piperazin-1-ylpyrimidine (Compound S16, 0.549 g, 2.16 mmol), tert-Butyl alcohol (13.2 mL, 138 mmol) and 1,4-Dioxane (3.37 mL, 43.1 mmol) was sparged with argon gas for 15 min at rt. tBuXPhos Pd G1 (0.062 g, 0.090 mmol) and sodium tert-butoxide (0.207 g, 2.16 mmol) were added and the mixture was sparged with argon gas for 10 min. The mixture was stirred at rt under argon overnight. The catalyst was removed by filtration through Celite® 545 and the filter cake was washed with 40 mL of CH₂Cl₂. The filtrate was washed with 2×40 mL of sat′d NaHCO₃ and 40 mL of brine. Drying over Na₂SO₄ and evaporation of the solvent gave 1.2 g of a red solid. Chromatography using a Biotage Isolera® in 0% acetone/CH₂Cl₂ to 50% acetone/CH₂Cl₂ over 12 column volumes on 20 g of silica gel gives 0.31 g (38%) of 3-[4-(5-benzylpyrimidin-2-yl)piperazin-1-yl]-6-(1-methyl-1H-pyrazol-4-yl)pyrazolo[1,5-b]pyridazine (Compound 8) as a yellow solid. ¹H NMR (300 MHz, CDCl₃): δ (ppm) 8.22 (s, 2H), 8.03 (d, J=2.9 Hz, 2H), 7.95 (d, J=9.2 Hz, 1H), 7.71 (s, 1H), 7.15-7.39 (m, 5H), 7.07 (d, J=9.3 Hz, 1H), 3.93-4.11 (m, 7H), 3.83 (s, 2H), 3.06-3.24 (m, 4H); MS (ESI) m/z [M+H]⁺ calcd. for C₂₅H₂₆N₉, 452.2; found, 452.3; HPLC purity: 210 nm: 96.8%; 254 nm: 100.0%.

### Example 12: Synthesis of Exemplary Compound 9

### 3-[4-(5-benzylpyrimidin-2-yl)piperazin-1-yl]-6-(1-methyl-1H-pyrazol-4-yl)[1, 2,3]triazolo[1,5-a]pyridine (Compound 9)

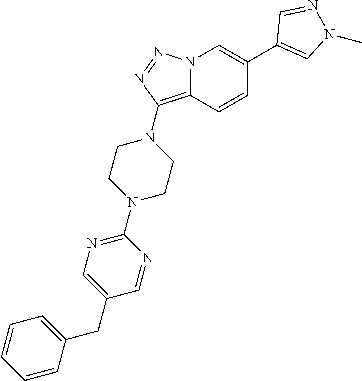 [SMILES: Cn1cc(-c2ccc3c(N4CCN(c5ncc(Cc6ccccc6)cn5)CC4)nnn3c2)cn1]

A flask containing 3-bromo-6-(1-methyl-1H-pyrazol-4-yl)[1,2,3]triazolo[1,5-a]pyridine (Compound S2, 500 mg, 2 mmol) and 5-benzyl-2-piperazin-1-ylpyrimidine (Compound S16, 549 mg, 2.16 mmol) was treated with tert-butyl alcohol (13 mL, 140 mmol) and 1,4-dioxane (3.3 mL, 42 mmol). The solution was sparged with argon gas for 15 min. The solution was treated with tBuXPhos Pd G1 (62 mg, 0.090 mmol) and sodium tert-butoxide (207 mg, 2.16 mmol) and the mixture was sparged again for 10 min with argon gas. The reaction mixture was stirred under an atmosphere of argon at rt for 15 minutes. The catalyst was removed by filtration through Celite®-545. The filter cake was washed with 40 mL of CH₂Cl₂. The filtrate was washed with two 25 mL portions of sat′d NaHCO₃ followed by 30 mL of brine, then dried over Na₂SO₄. The solution was filtered and concentrated to yield 1.1 g of a dark paste. The crude material was purified by chromatography using a Biotage Isolera® system in 0%-50% acetone/CH₂Cl₂ over 12 column volumes on 12 g of silica gel. The isolated product was dried in vacuo to give 0.12 g (14%) of 3-[4-(5-benzylpyrimidin-2-yl)piperazin-1-yl]-5-(1-methyl-1H-pyrazol-4-yl)[1,2,3]triazolo[1,5-a]pyridine (Compound 9) as an off white foam. ¹H NMR (300 MHz, DMSO-d₆): δ (ppm) 9.13 (s, 1H), 8.27-8.39 (m, 3H), 8.07 (s, 1H), 7.97 (d, J=9.3 Hz, 1H), 7.40 (d, J=9.2 Hz, 1H), 7.10-7.36 (m, 5H), 3.86-4.00 (m, 7H), 3.80 (s, 2H), 3.33-3.47 (m, 4H); MS (ESI) m/z [M+H]⁺ calcd. for C₂₅H₂₆N₉, 452.2; found, 452.3; HPLC purity: 210 nm: 97.0%; 254 nm: 97.0%.

### Example 13: Synthesis of Exemplary Compound 10

### 6-(1-methyl-1H-pyrazol-4-yl)-3-(4-pyrimidin-2-ylpiperazin-1-yl)pyrazolo[1,5-a]pyridine (Compound 10)

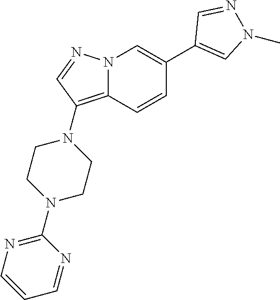 [SMILES: Cn1cc(-c2ccc3c(N4CCN(c5ncccn5)CC4)cnn3c2)cn1]

To a round bottom flask containing 6-(1-methyl-1H-pyrazol-4-yl)-3-piperazin-1-ylpyrazolo[1,5-a]pyridine (Compound 12, 136 mg, 0.48 mmol) and 2-chloropyrimidine (72 mg, 0.63 mmol) was added N-methylpyrrolidinone (2.72 mL). Potassium carbonate (166 mg, 1.20 mmol) was added, and the mixture was heated at 110° C. for 17 h. It was cooled to ambient temperature and partitioned between DCM and water. The organic layer was washed with saturated aqueous sodium bicarbonate, and brine, dried (Na₂SO₄), filtered, and concentrated. The residue was purified by chromatography (12 g Aq C18 reverse-phase cartridge) eluting with a gradient of 20-70% MeCN in water (no acid additive). The solids obtained were dried in vacuo to afford 112 g, (64%) of 6-(1-methyl-1H-pyrazol-4-yl)-3-(4-pyrimidin-2-ylpiperazin-1-yl)pyrazolo[1,5-a]pyridine (Compound 10) as a tan solid. ¹H NMR (400 MHZ, CDCl₃): δ (ppm) 8.47 (s, 1H), 8.35 (d, J=4.77 Hz, 2H), 7.74 (d, J=9.54 Hz, 2H), 7.62 (s, 1H), 7.57 (d, J=9.17 Hz, 1H), 7.15 (dd, J=9.17, 1.34 Hz, 1H), 6.53 (t, J=4.71 Hz, 1H), 4.05 (m, 4H), 3.93 (s, 3H), 3.14 (m, 4H); MS (ESI+, m/z): MS (ESI) m/z [M+H]⁺ calcd. for C₁₉H₂₁N₈, 361.2; found, 361.2; HPLC purity: 210 nm: 100.0%; 254 nm: 100.0%.

### Example 14: Synthesis of Exemplary Compound 11

### tert-butyl 4-[6-(1-methyl-1H-pyrazol-4-yl)pyrazolo[1,5-a]pyridin-3-yl]piperazine-1-carboxylate (Compound 11)

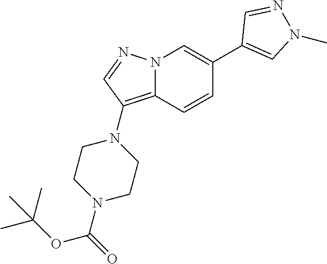 [SMILES: Cn1cc(-c2ccc3c(N4CCN(C(=O)OC(C)(C)C)CC4)cnn3c2)cn1]

To a round bottom flask containing 3-bromo-6-(1-methyl-1H-pyrazol-4-yl)pyrazolo[1,5-a]pyridine (Compound S9, 4.00 g, 14.4 mmol) and tert-butyl-1-piperazinecarboxylate (3.23 g, 17.3 mmol) was added tert-butyl alcohol (202 mL) and 1,4-dioxane (124 mL). The solution was sparged with nitrogen for 20 min at 25° C. To the solution was added tBuXPhos Pd G1 (248 mg, 5.41 mmol) and sodium tert-butoxide (520 mg, 0.54 mmol) and the reaction mixture was sparged with nitrogen for an additional 10 min at 25° C. The flask was heated in a 55° C. bath for 1.5 h, cooled to ambient temperature and diluted with ethyl acetate. The mixture was washed with water and brine, dried (Na₂SO₄), filtered, and concentrated. The residue was purified by Biotage chromatography (120 g normal phase cartridge) eluting with a gradient of 10-90% EtOAc in DCM. The solids obtained were dried in vacuo to afford 1.62 g (29%) of tert-butyl 4-[6-(1-methyl-1H-pyrazol-4-yl)pyrazolo[1,5-a]pyridin-3-yl]piperazine-1-carboxylate (Compound 11) as a yellow solid. ¹H NMR (400 MHZ, CDCl₃): δ (ppm) 8.46 (s, 1H), 7.74 (s, 1H), 7.70 (s, 1H), 7.63 (s, 1H), 7.50 (d, J=7.3 Hz, 1H), 7.14 (d, J=1.47 Hz, 1H), 4.03-4.01 (m, 4H), 3.98 (s, 3H), 3.64 (m, 4H), 3.02 (m, 4H), 1.50 (s, 9H); MS (ESI) m/z [M+H]⁺ calcd. for C₂₀H₂₇N₆O₂, 383.2; found, 383.3; HPLC purity: 210 nm: 99.4%; 254 nm: 99.2%.

### Example 15: Synthesis of Exemplary Compound 12

### 6-(1-methyl-1H-pyrazol-4-yl)-3-piperazin-1-ylpyrazolo[1,5-a]pyridine (Compound 12)

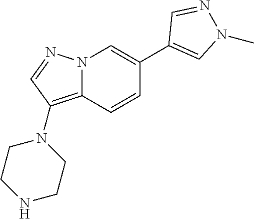 [SMILES: Cn1cc(-c2ccc3c(N4CCNCC4)cnn3c2)cn1]

To a round bottom flask containing tert-butyl 4-[6-(1-methyl-1H-pyrazol-4-yl)pyrazolo[1,5-a]pyridin-3-yl]piperazine-1-carboxylate (Compound 11, 0.340 g, 0.89 mmol) was added dichloromethane (6.80 mL). The solution was cooled on an ice bath and trifluoroacetic acid (1.70 mL, 22.1 mmol) was added slowly. After an hour solvent was removed by rotoevaporation iteratively with tert-butylmethyl ether as an additive to remove excess trifluoroacetic acid. The residue was dissolved with dichloromethane and swirled with saturated aqueous sodium bicarbonate diluted with water, and tert-butylmethyl ether was added so to flip the layers. The aqueous was then extracted with more tert-butylmethyl ether to remove impurities. It was then extracted with dichloromethane to remove the product. The dichloromethane layer was dried with sodium sulfate and solvent was removed under vacuum to obtain 105 mg (42%) of 6-(1-methyl-1H-pyrazol-4-yl)-3-piperazin-1-ylpyrazolo[1,5-a]pyridine (Compound 12) as a yellow solid. ¹H NMR (400 MHZ, CDCl₃): δ (ppm) 8.45 (s, 1H), 7.74 (s, 1H), 7.70 (s, 1H), 7.60 (s, 1H), 7.52 (d, J=9.17 Hz, 1H), 7.11 (d, J=9.05 Hz, 1H), 3.97 (s, 3H), 3.08 (bs, 5H), 3.06 (bs, 4H); MS (ESI+, m/z): Calcd. for C₁₅H₁₉N₆: 283.2. Found: 283.1; HPLC purity: 210 nm: 100%; 254 nm: 100%.

### Example 16: Synthesis of Exemplary Compound 13

### Benzyl 4-[6-(1-methyl-1H-pyrazol-4-yl)pyrazolo[1,5-a]pyridin-3-yl]piperazine-1-carboxylate (Compound 13)

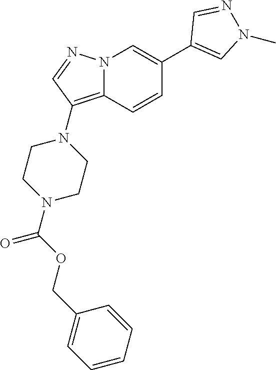 [SMILES: Cn1cc(-c2ccc3c(N4CCN(C(=O)OCc5ccccc5)CC4)cnn3c2)cn1]

To a glass vial containing 6-(1-methyl-1H-pyrazol-4-yl)-3-piperazin-1-ylpyrazolo[1,5-a]pyridine dihydrochloride (Compound S19, 50 mg, 0.14 mmol) cooled at 1° C. was added DCM (1.00 mL) and triethylamine (98 μL, 0.70 mmol). Benzyl chloroformate (20 μL, 0.14 mmol) was added dropwise and the mixture warmed to ambient temperature over 16 hours. The reaction mixture was diluted with dichloromethane and was purified by Biotage chromatography (4 g normal phase cartridge) eluting with a gradient of 0-90% acetone in DCM.

The above reaction and purification were repeated on 100 mg (0.28 mmol scale) and additional quantities of benzyl chloroformate were added to fully consume starting amine as determined by HPLC.

The solids after chromatography were combined and dried in vacuo to afford 104 mg (59%) of benzyl 4-[6-(1-methyl-1H-pyrazol-4-yl)pyrazolo[1,5-a]pyridin-3-yl]piperazine-1-carboxylate (Compound 13) as a yellow solid. ¹H NMR (400 MHZ, DMSO-d₆): δ (ppm) 8.82 (s, 1H), 8.22 (s, 1H), 7.96 (s, 1H), 7.76 (s, 1H), 7.63 (s, 1H), 7.64 (d, J=9.2 Hz, 1H), 7.35 (m, 4H), 7.27 (m, 2H), 5.12 (s, 2H), 3.86 (s, 3H), 3.60 (br. s, 4H), 2.97 (br. s, 4H); MS (ESI) m/z [M+H]⁺ calcd. for C₂₃H₂₅N₆O₂: 417.2. Found: 417.3; HPLC purity: 210 nm: 100%; 254 nm: 100%.

### Example 17: Synthesis of Exemplary Compound 14

### Tetrahydro-2H-pyran-4-ylmethyl 4-[6-(1-methyl-1H-pyrazol-4-yl)pyrazolo[1,5-a]pyridin-3-yl]piperazine-1-carboxylate (Compound 14)

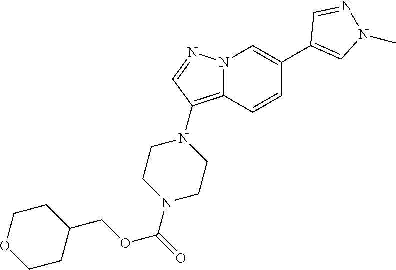 [SMILES: Cn1cc(-c2ccc3c(N4CCN(C(=O)OCC5CCOCC5)CC4)cnn3c2)cn1]

To a solution of 4-nitrophenyl chlorocarbonate (47 mg, 0.23 mmol) in DCM (2.00 mL) and triethylamine (184 μL, 1.32 mmol) was added tetrahydro-2H-pyran-4-ylmethanol (27 mg, 0.23 mmol). The solution was stirred at room temperature for an hour and 6-(1-methyl-1H-pyrazol-4-yl)-3-piperazin-1-ylpyrazolo[1,5-a]pyridine dihydrochloride (Compound S19, 70 mg, 0.22 mmol) was added. The reaction was monitored by HPLC and if necessary, was dosed with additional carbonic acid 4-nitro-phenyl ester tetrahydro-pyran-4-ylmethyl ester until all of 6-(1-methyl-1H-pyrazol-4-yl)-3-piperazin-1-ylpyrazolo[1,5-a]pyridine was consumed. Upon completion, solvent was removed, and the residue was purified by Biotage chromatography (12 g cartridge) eluting with a gradient of acetone in DCM with hexanes as an additive. This was followed by a gradient of acetone in DCM with no hexanes to afford 56 mg (60%) of tetrahydro-2H-pyran-4-ylmethyl 4-[6-(1-methyl-1H-pyrazol-4-yl)pyrazolo[1,5-a]pyridin-3-yl]piperazine-1-carboxylate (Compound 14) as a yellow solid. ¹H NMR (400 MHZ, CDCl₃): δ (ppm) 8.48 (br. s., 1H) 7.75 (s, 1H) 7.72 (br. s., 1H) 7.62 (s, 1H) 7.51 (d, J=9.05 Hz, 1H) 7.15 (d, J=9.05 Hz, 1H) 4.01 (d, J=6.48 Hz, 4H) 3.98 (s, 3H) 3.69 (t, J=4.83 Hz, 4H) 3.42 (td, J=11.8 Hz, J=2.02 Hz, 2H) 3.04 (br. s., 2H); 1.96 (m, 1H), 1.66 (m, 2H), 1.43 (m, 2H). MS (ESI) m/z [M+H]⁺ calcd. for C₂₂H₂₉N₆O₃: 425.2, Found: 425.3; HPLC purity: 210 nm: 97.18%; 254 nm: 97.3%.

### Example 18: Synthesis of Exemplary Compound 15

### Preparation of 2-(tetrahydro-2H-pyran-4-yl)ethyl 4-[6-(1-methyl-1H-pyrazol-4-yl)pyrazolo[1,5-a]pyridin-3-yl]piperazine-1-carboxylate (Compound 15)

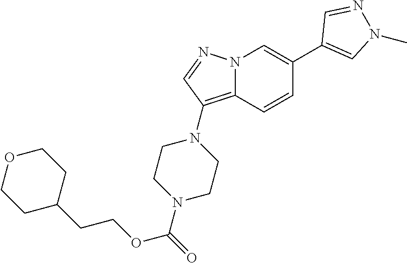 [SMILES: Cn1cc(-c2ccc3c(N4CCN(C(=O)OCCC5CCOCC5)CC4)cnn3c2)cn1]

Compound 15 was prepared as described for the preparation of tetrahydro-2H-pyran-4-ylmethyl 4-[6-(1-methyl-1H-pyrazol-4-yl)pyrazolo[1,5-a]pyridin-3-yl]piperazine-1-carboxylate (Example 17, Compound 14) using 2-(tetrahydro-2H-pyran-4-yl)ethanol (30 mg, 0.27 mmol) to afford 63 mg (73%) of 2 (tetrahydro-2H-pyran-4-yl)ethyl 4-[6-(1-methyl-1H-pyrazol-4-yl)pyrazolo[1,5-a]pyridin-3-yl]piperazine-1-carboxylate (Compound 15) as a yellow solid. ¹H NMR (400 MHZ, CDCl₃): δ (ppm) 8.47 (br. s., 1H) 7.75 (s, 1H) 7.71 (br. s., 1H) 7.62 (s, 1H) 7.51 (br. s., 1H) 7.16 (br. s., 1H) 4.19 (t, J=6.30 Hz, 2H) 3.97 (m, 5H) 3.69 (br. s., 4H) 3.40 (td, J=11.8 Hz, J=1.65 Hz, 2H) 3.04 (br. s., 4H); 1.65 (m, 5H), 1.36 (m, 2H). MS (ESI) m/z [M+H]⁺ calcd. for C₂₃H₃₁N₆O₃: 439.2, Found: 439.3; HPLC purity: 210 nm: 100%; 254 nm: 100%.

### Example 19: Synthesis of Exemplary Compound 16

### 1-ethylpropyl 4-[6-(1-methyl-1H-pyrazol-4-yl)pyrazolo[1,5-a]pyridin-3-yl]piperazine-1-carboxylate (Compound 16)

### Step 1. Preparation of 1-ethylpropyl 4-nitrophenyl carbonate

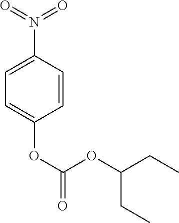 [SMILES: CCC(CC)OC(=O)Oc1ccc([N+](=O)[O-])cc1]

To a cooled solution of 3-pentanol (44 mg, 0.50 mmol) in DCM (5.2 mL) at 3° C. was added pyridine (170 μL, 2.10 mmol) and after stirring 10 minutes 4-nitrophenyl chlorocarbonate (0.202 g, 1.00 mmol). Cooling was stopped, and the solution stirred at room temperature overnight.

The mixture was concentrated under vacuum and the residue was azeotroped with heptane to remove pyridine. The residue was purified by Biotage chromatography (12 g) cartridge eluting with a gradient of MTBE in hexanes to afford 0.118 g (93%) of 1-ethylpropyl 4-nitrophenyl carbonate as a clear oil. ¹H NMR (400 MHZ, CDCl₃): δ (ppm) 8.29 (d, J=9.17 Hz, 2H) 7.40 (d, J=9.17 Hz, 2H) 4.74 (t, J=6.17 Hz, 1H) 1.63-1.85 (m, 4H) 1.00 (t, J=7.46 Hz, 6H).

### Step 2. Preparation of 1-ethylpropyl 4-[6-(1-methyl-1H-pyrazol-4-yl)pyrazolo[1,5-a]pyridin-3-yl]piperazine-1-carboxylate (Compound 16)

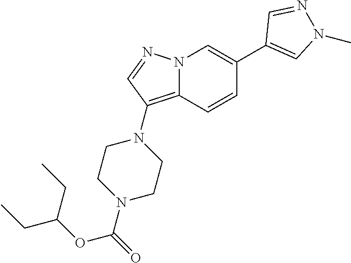 [SMILES: CCC(CC)OC(=O)N1CCN(c2cnn3cc(-c4cnn(C)c4)ccc23)CC1]

To a solution of 1-ethylpropyl 4-nitrophenyl carbonate (118 mg, 0.47 mmol) in MeCN (4 mL) was added diisopropylethylamine (1.4 mL, 8.0 mmol) and 6-(1-methyl-1H-pyrazol-4-yl)-3-piperazin-1-ylpyrazolo[1,5-a]pyridine dihydrochloride (Compound S19, 100 mg, 0.28 mmol). After stirring overnight, the volatiles were removed by rotoevaporation. The residue was purified by Biotage chromatography (12 g normal phase cartridge) eluting with a gradient of acetone (containing 0.1% TEA) in hexanes to afford 75 mg (67%) of 1-ethylpropyl 4-[6-(1-methyl-1H-pyrazol-4-yl)pyrazolo[1,5-a]pyridin-3-yl]piperazine-1-carboxylate (Compound 16) as a beige solid. ¹H NMR (400 MHZ, CDCl₃): δ (ppm) 8.46 (s, 1H) 7.74 (s, 1H) 7.71 (s, 1H), 7.61 (s, 1H) 7.51 (s, 1H) 7.15 (dd, J=9.17, 1.34 Hz, 1H) 4.71 (t, 1H) 3.98 (s, 3H) 3.70 (m, 4H) 3.04 (m, 4H) 1.62 (m, 4H) 0.93 (t, J=7.46, 6H); MS (ESI) m/z [M+H]⁺ calcd. for C₂₁H₂₉N₆O₂: 397.2, Found: 397.3; HPLC purity: 210 nm: 100.0%; 254 nm: 100.0%.

Using the procedures described in Example 19, Compound 16 and reagents, starting materials, and conditions known to those skilled in the art were used to prepare the compounds shown in Table A.

TABLE A

| Compound | Name | Characterization |
|---|---|---|
| Compound 307 | 3,3-difluorocyclobutyl 4-[6- | ¹H NMR (400 MHz, DMSO-d₆) d ppm |
| | (1-methyl-1H-pyrazol-4- | 8.82 (s, 1H), 8.82 (s, 1H), 7.97 (s, 1H), |
| | yl)pyrazolo[1,5-a]pyridin-3- | 7.77 (s, 1H), 7.65 (d, J = 9 Hz, 1H), 7.33 |
| | yl]piperazine-1-carboxylate | (dd, J = 9, 2 Hz, 1H), 4.85 (m, 1H), 3.87 |
| | | (s, 3H), 3.57 (br. s., 4H), 3.05 (br s, 2H), |
| | | 2.97 (m, 4H), 2.71 (m, 2H); ¹⁹F NMR |
| | | (376 MHz DMSO-d₆) d ppm −83.9 (d, J = |
| | | 196 Hz) −94.1 (d, J = 196 Hz); MS (ESI) |
| | | m/z [M + H]⁺ calcd. for C₂₀H₂₃F₂N₆O₂: |
| | | 417.2, Found: 417.1; HPLC purity: 210 |
| | | nm: 99.0%; 254 nm: 99.1%. |
| Compound 308 | (3,3- | ¹H NMR (400 MHz, CDCl₃) δ ppm 8.47 |
| | difluorocyclobutyl)methyl 4- | (s, 1H), 7.75 (s, 1H), 7.72 (s, 1H), 7.55 |
| | [6-(1-methyl-1H-pyrazol-4- | (br s, 1H), 7.17 (d, J = 9 Hz, 1H), 4.20 (d, |
| | yl)pyrazolo[1,5-a]pyridin-3- | J = 6 Hz, 2H), 3.98 (s, 3H), 3.72 (br s, 4H), |
| | yl]piperazine-1-carboxylate | 3.07 (br s, 4H), 2.71 (m, 2H), 2.52 (m, |
| | | 1H), 2.41 (m, 2H); ¹⁹F NMR (376 MHz |
| | | CDCl₃) 8 ppm −83.8 (d, J = 200 Hz) −93.3 |
| | | (d, J = 194 Hz); MS (ESI) m/z [M + H]⁺ |
| | | calcd. for C₂₁H₂₅F₂N₆O₂: 431.2, Found: |
| | | 431.1; HPLC purity: 210 nm: 100.0%; |
| | | 254 nm: 100.0%. |
| Compound 309 | 3 methylcyclobutyl 4-[6-(1- | ¹H NMR (400 MHz, CDCl₃, ca. 1:6 |
| | methyl-1H-pyrazol-4- | mixture of diastereomers) δ ppm 8.47 (s, |
| | yl)pyrazolo[1,5-a]pyridin-3- | 1H), 8.82 (s, 1H), 7.74 (s, 1H), 7.72 (s, |
| | yl]piperazine-1-carboxylate | 1H), 7.61 (s, 1H), 7.54 (br s, 1H), 7.16 (d, |
| | | J = 9.2, 1H), [5.13 (q, J = 7 Hz, 0.14H), |
| | | 4.82 (q, J = 7 Hz, 0.84H)], (3.98 (s, 3H), |
| | | 3.70 (br s, 4H), 3.05 (br s, 4H), [1.55 to |
| | | 2.65 (5H)] [1.17 (d, J = 7 Hz, 0.51H), 1.13 |
| | | (d, J = 7 Hz, 2.50H)]; MS (ESI) m/z |
| | | [M + H]⁺ calcd. for C₂₁H₂₇N₆O₂⁺: 395.2, |
| | | Found: 395.1; HPLC purity: 210 nm: |
| | | 100.0%; 254 nm: 100.0%. |
| Compound 310 | (3-methylcyclobutyl)methyl | ¹H NMR (400 MHz, DMSO-d₆, ca. 1:1 |
| | 4-[6-(1-methyl-1H-pyrazol-4- | mixture of diastereomers) δ ppm 8.82 (s, |
| | yl)pyrazolo[1,5-a]pyridin-3- | 1H), 8.22 (s, 1H), 7.96 (s, 1H), 7.76 (s, |
| | yl]piperazine-1-carboxylate | 1H), 7.65 (dd, J = 9, 1 Hz, 1H), 7.32 (dd, |
| | | J = 9, 1 Hz, 1H) [4.05 (d, J = 7 Hz, 1H), |
| | | 3.95 (d, J = 6 Hz, 1H)], 3.87 (s, 3H), 3.56 |
| | | (br s, 4H), 2.96 (br s, 4H), [1.28 to 2.60 |
| | | (6H)] [1.08 (d, J = 7 Hz, 0.91H), 1.00 (d, |
| | | J = 7 Hz, 1H)]; MS (ESI) m/z [M + H]⁺ |
| | | calcd. for C₂₂H₂₉N₆O₂⁺: 409.2, Found: |
| | | 409.2. |
| Compound 311 | (1R)-1-(3,4- | ¹H NMR (400 MHz, CDCl₃) δ ppm 8.47 |
| | dichlorophenyl)ethyl 4-[6-(1- | (s, 1H), 7.75 (s, 1H), 7.71 (s, 1H), 7.62 (s, |
| | methyl-1H-pyrazol-4- | 1H), 7.52 (d, J = 8 Hz, 1H), 7.46 (d, J = 2 |
| | yl)pyrazolo[1,5-a]pyridin-3- | Hz, 1H), 7.44 (d, J = 8 Hz, 1H), 7.20 (dd, |
| | yl]piperazine-1-carboxylate | J = 8, 2 Hz, 1H), 7.16 (dd, J = 9, 1 Hz, 1H), |
| | | 5.79 (q, J = 7 Hz, 1H), 3.98 (s, 3H), 3.72 |
| | | (br s, 4H), 3.05 (br s, 4H), 1.56 (d, J = 7 |
| | | Hz, 3H); MS (ESI) m/z [M + H]⁺ calcd. for |
| | | C₂₄H₂₅C₁₂N₆O₂⁺: 499.1, Found: 499.1; |
| | | HPLC purity: 210 nm: 100.0%; 254 nm: |
| | | 100.0%. |
| Compound 312 | (1R)-1-(2-naphthyl)ethyl 4-[6- | ¹H NMR (400 MHz, CDCl₃) δ ppm 8.47 |
| | (1-methyl-1H-pyrazol-4- | (s, 1H), 7.36 (m, 3H), 7.83 (d, J = 2 Hz, |
| | yl)pyrazolo[1,5-a]pyridin-3- | 1H), 7.74 (s, 1H), 7.71 (s, 1H), 7.61 (s, |
| | yl]piperazine-1-carboxylate | 1H), 7.50 (m, 4H), 7.15 (dd, J = 9, 1 Hz, |
| | | 1H), 6.04 (q, J = 7 Hz, 1H), 3.98 (s, 3H), |
| | | 3.78 (br s, 4H), 3.05 (br s, 2H), 1.68 (d, |
| | | J = 7 Hz, 3H); MS (ESI) m/z [M + H]⁺ |
| | | calcd. for C₂₈H₂₉N₆O₂⁺: 481.2, Found: |
| | | 481.2; HPLC purity: 210 nm: 100.0%; |
| | | 254 nm: 100.0%. |
| Compound 313 | (1R)-1-[4- | ¹H NMR (400 MHz, CDCl₃) δ ppm 8.47 |
| | (trifluoromethyl)phenyl]ethyl | (s, 1H), 7.75 (d, J = 1 Hz, 1H), 7.72 (s, |
| | 4-[6-(1-methyl-1H-pyrazol-4- | 1H), 7.65 (s, 1H), 7.62 (d, J = 2.8, 2H), |
| | yl)pyrazolo[1,5-a]pyridin-3- | 7.54 (br s, 1H), 7.48 (d, J = 8 Hz, 2H), |
| | yl]piperazine-1-carboxylate | 7.17 (d, J = 9 Hz, 1H), 5.90 (q, J = 7 Hz, |
| | | 1H), 3.98 (s, 3H), 3.75 (br s, 4H), 3.07 (br |
| | | s, 4H), 1.59 (d, J = 7 Hz, 3H); ¹⁹F NMR |
| | | (376 MHz CDCl₃) d ppm −62.5; MS |
| | | (ESI) m/z [M + H]⁺ calcd. for |
| | | C₂₅H₂₆F₃N₆O₂⁺: 499.2, Found: 499.2; |
| | | HPLC purity: 210 nm: 100.0%; 254 nm: |
| | | 100.0%. |
| Compound 314 | (1R)-5-chloro-2,3-dihydro- | ¹H NMR (400 MHz, DMSO-d₆) δ ppm |
| | 1H-inden-1-yl 4-[6-(1-methyl- | 8.81 (s, 1H), 8.21 (s, 1H), 7.96 (s, 1H), |
| | 1H-pyrazol-4- | 7.75 (s, 1H), 7.63 (d, J = 9 Hz, 1H), 7.43 |
| | yl)pyrazolo[1,5-a]pyridin-3- | (d, J = 8 Hz, 1H), 7.38 (s, 1H), 7.30 (m, |
| | yl]piperazine-1-carboxylate | 2H), 6.01 (dd, J = 7, 4 Hz, 1H), 3.86 (s, |
| | | 3H), 3.55 (br s, 4H), 3.03 (m, 1H), 2.95 |
| | | (br s, 4H), 2.86 (m, 1H), 2.48 (m, 1H), |
| | | 2.05 (m, 1H); MS (ESI) m/z [M + H]⁺ |
| | | calcd. for C₂₅H₂₆ClN₆O₂⁺: 477.2, Found: |
| | | 477.2; HPLC purity: 210 nm: 100.0%; |
| | | 254 nm: 100.0%. |
| Compound 315 | (1R)-1-pyridin-3-ylethyl 4-[6- | ¹H NMR (400 MHz, CDCl₃) δ ppm 8.67 |
| | (1-methyl-1H-pyrazol-4- | (s, 1H), 8.57 (d, J = 4 Hz, 1H), 8.46 (s, |
| | yl)pyrazolo[1,5-a]pyridin-3- | 1H), 7.74 (s, 1H), 7.70 (s, 1H), 7.68 (t, |
| | yl]piperazine-1-carboxylate | J = 2 Hz, 1H), 7.61 (s, 1H), 7.50 (d, J = 9 |
| | | Hz, 1H), 7.31 (dd, J = 8, 5 Hz, 1H), 7.15 |
| | | (d, J = 9, 1 Hz, 1H), 5.90 (q, J = 7 Hz, 1H), |
| | | 3.97 (s, 3H), 3.71 (br s, 4H), 3.04 (br s, |
| | | 4H), 1.62 (d, J = 7 Hz, 3H); MS (ESI) m/z |
| | | [M + H]⁺ calcd. for C₂₄H₂₇N₆O₂⁺: 432.2, |
| | | Found: 432.3; HPLC purity: 210 nm: |
| | | 100.0%; 254 nm: 100.0%. |
| Compound 316 | 4-cyanobenzyl 4-[6-(1- | ¹H NMR (400 MHz, CDCl₃) δ ppm 8.47 |
| | methyl-1H-pyrazol-4- | (s, 1H), 7.75 (d, J = 0.5 Hz, 1H), 7.71 (s, |
| | yl)pyrazolo[1,5-a]pyridin-3- | 1H), 7.69 (m, 1H), 7.67 (m, 1H), 7.62 (s, |
| | yl]piperazine-1-carboxylate | 1H), 7.52 (br s, 1H), 7.50 (s 3H), 7.48 (s, |
| | | 2H), 7.17 (d, J = 1.5 Hz, 1H), 7.15 (d, |
| | | J = 1.5 Hz, 1H), 5.23 (s, 2H), 3.98 (s, 3H), |
| | | 3.74 (m, 4H), 3.06 (br s, 4H); MS (ESI) |
| | | m/z [M + H]⁺ calcd. for C₂₄H₂₄N₇O₂⁺: |
| | | 442.2, Found: 442.2; HPLC purity: 210 |
| | | nm: 100.0%; 254 nm: 100.0%. |
| Compound 317 | 1-(6-methylpyridin-3-yl)ethyl | ¹H NMR (400 MHz, CDCl₃) δ ppm 8.54 |
| | 4-[6-(1-methyl-1H-pyrazol-4- | (d, J = 2.2 Hz, 1H), 8.46 (s, 1H), 7.74 (s, |
| | yl)pyrazolo[1,5-a]pyridin-3- | 1H), 7.70 (s, 1H), 7.61 (s, 2H), 7.59 (d, |
| | yl]piperazine-1-carboxylate | J = 2.1 Hz, 1H), 7.50 (dd, J = 9.2, 0.7 Hz, |
| | | 1H), 7.27 (s, 2H), 7.16 (m, 2H), 5.90 (q, |
| | | J = 7 Hz, 1H), 3.98 (s, 3H), 3.70 (br s, 4H), |
| | | 3.03 (br s, 4H), 2.58 (s, 3H), 1.60 (d, |
| | | J = 6.7 Hz, 3H); MS (ESI) m/z [M + H]⁺ |
| | | calcd. for C₂₄H₂₈N₇O₂: 446.2, Found: |
| | | 446.3; HPLC purity: 210 nm: 98.9%; 254 |
| | | nm: 98.7%. |

### Example 20: Synthesis of Exemplary Compound 17

### 3-[4-(3,3-dimethylbutanoyl)piperazin-1-yl]-6-(1-methyl-1H-pyrazol-4-yl)pyrazolo[1,5-a]pyridine (Compound 17)

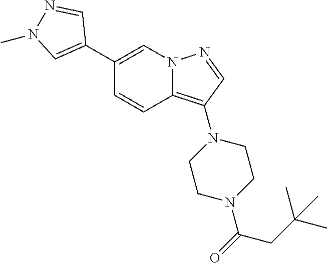 [SMILES: Cn1cc(-c2ccc3c(N4CCN(C(=O)CC(C)(C)C)CC4)cnn3c2)cn1]

A vial containing 6-(1-methyl-1H-pyrazol-4-yl)-3-piperazin-1-ylpyrazolo[1,5-a]pyridine hydrochloride (Compound S19, 146 mg, 0.66 mmol) suspended in DCM (1.5 mL) was cooled in an ice bath to 0° C. To the mixture was added Et₃N (139 mg, 1.38 mmol) followed by a solution of t-butylacetyl chloride (26 mg, 0.16 mmol) in DCM (1.5 mL). The ice bath was removed, and the reaction stirred 1h. To the reaction was added H₂O (4 mL). The resulting two layers were separated. The aqueous layer was extracted three times with DCM (3×4 mL). The combined DCM layers were dried (Na₂SO₄), filtered and concentrated. The residue was purified by ISCO chromatography (12 g normal phase cartridge) eluting with a gradient of 0-10% IPA in EtOAc. The solids obtained were triturated with hexane and concentrated in vacuo. This was repeated three more times. The resulting solid was dried in vacuo to afford 98 mg (56%) of 3-[4-(3,3-dimethylbutanoyl)piperazin-1-yl]-6-(1-methyl-1H-pyrazol-4-yl)pyrazolo[1,5-a]pyridine (Compound 17) as a yellow solid. ¹H NMR (400 MHZ, CDCl₃): δ (ppm) 8.48 (s, 1H) 7.76 (d, J=0.49 Hz, 1H) 7.72 (s, 1H) 7.63 (s, 1H) 7.52 (dd, J=9.17, 0.61 Hz, 1H) 7.17 (dd, J=9.17, 1.47 Hz, 1H) 3.99 (s, 3H) 3.83-3.91 (m, 2H) 3.69-3.78 (m, 2H) 3.02-3.12 (m, 4H) 2.35 (s, 2H) 1.11 (s, 9H). MS (ESI) m/z [M+H]⁺ calcd. for C₂₁H₂₉N₆O: 381.2. Found: 381.3; HPLC purity: 210 nm: 100.0%; 254 nm: 100.0%.

### Example 21: Synthesis of Exemplary Compound 18

### 6-(1-methyl-1H-pyrazol-4-yl)-3-[4-(phenylacetyl)piperazin-1-yl]pyrazolo[1,5-a]pyridine (Compound 18)

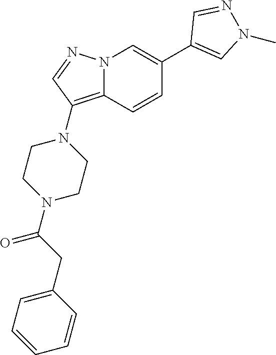 [SMILES: Cn1cc(-c2ccc3c(N4CCN(C(=O)Cc5ccccc5)CC4)cnn3c2)cn1]

Compound 18 was prepared as described for the preparation of 2,2-dimethylpropyl 4-[6-(1-methyl-1H-pyrazol-4-yl)pyrazolo[1,5-a]pyridin-3-yl]piperazine-1-carboxylate (Example 20, Compound 17) using benzeneacetyl chloride (31 μL, 0.23 mmol) to afford 59 mg (70%) of 6-(1-methyl-1H-pyrazol-4-yl)-3-[4-(phenylacetyl)piperazin-1-yl]pyrazolo[1,5-a]pyridine (Compound 18) as a solid. ¹H NMR (400 MHz, DMSO-d₆): δ (ppm) 8.22 (s, 1H), 7.96 (s, 1H), 7.72 (s, 1H), 7.64 (d, J=9.29 Hz, 1H), 7.32 (m, 3H), 7.25 (m, 3H), 3.86 (s, 3H), 3.77 (s, 2H), 3.67 (m, 4H), 2.92 (m, 4H); MS (ESI) m/z [M+H]⁺ calcd. for C₂₃H₂₅N₆O: 401.2. Found: 401.2; HPLC purity: 210 nm: 97.0%; 254 nm: 98.0%.

### Example 22: Synthesis of Exemplary Compound 19

### 1-methylpiperidin-4-yl 4-[6-(1-methyl-1H-pyrazol-4-yl)pyrazolo[1,5-a]pyridin-3-yl]piperazine-1-carboxylate (Compound 19)

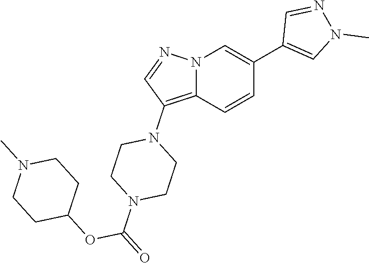

Compound 19 was prepared as described for the preparation of tetrahydro-2H-pyran-4-ylmethyl 4-[6-(1-methyl-1H-pyrazol-4-yl)pyrazolo[1,5-a]pyridin-3-yl]piperazine-1-carboxylate (Example 17, Compound 14) using 4-hydroxy-N-methylpiperidine (30 mg, 0.26 mmol). The product from chromatography was further purified by saturated aqueous sodium bicarbonate wash as a solution in ethyl acetate to afford 55 mg (58%) of 1-methylpiperidin-4-yl 4-[6-(1-methyl-1H-pyrazol-4-yl)pyrazolo[1,5-a]pyridin-3-yl]piperazine-1-carboxylate as a solid. ¹H NMR (400 MHZ, CDCl₃): δ (ppm) 8.47 (s, 1H) 7.74 (s, 1H) 7.71 (s, 1H) 7.61 (s, 1H) 7.51 (dd, J=9.17, 0.61 Hz, 1H) 7.15 (dd, J=9.17, 1.47 Hz, 1H) 4.77 (m, 1H) 3.98 (s, 3H) 3.69 (m, 4H) 3.04 (m, 4H); 2.63 (br. s., 2H) 2.30 (m, 5H), 1.97 (m, 2H), 1.78 (m, 2H). MS (ESI) m/z [M+H]⁺ calcd. for C₂₂H₃₉N₇O₂: 424.2, Found: 424.3; HPLC purity: 210 nm: 99.4%; 254 nm: 99.4%.

### Example 23: Synthesis of Exemplary Compound 20

### (1-methylpiperidin-4-yl)methyl 4-[6-(1-methyl-1H-pyrazol-4-yl)pyrazolo[1,5-a]pyridin-3-yl]piperazine-1-carboxylate (Compound 20)

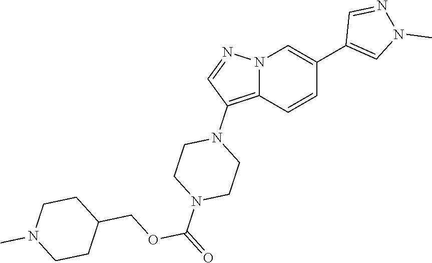

Compound 20 was prepared as described for the preparation of tetrahydro-2H-pyran-4-ylmethyl 4-[6-(1-methyl-1H-pyrazol-4-yl)pyrazolo[1,5-a]pyridin-3-yl]piperazine-1-carboxylate (Example 17, Compound 14) using (1-methylpiperidin-4-yl)methanol (33 mg, 0.26 mmol). The product from chromatography was further purified by saturated aqueous sodium bicarbonate wash as a solution in ethyl acetate to afford 58 mg (59%) of (1-methylpiperidin-4-yl)methyl 4-[6-(1-methyl-1H-pyrazol-4-yl)pyrazolo[1,5-a]pyridin-3-yl]piperazine-1-carboxylate as a solid. ¹H NMR (400 MHZ, CDCl₃): δ (ppm) 8.46 (s, 1H) 7.75 (s, 1H) 7.70 (s, 1H) 7.61 (s, 1H) 7.50 (d, J=9.05 Hz, 1H) 7.15 (dd, J=9.17, 1.34 Hz, 1H) 4.01 (d, J=5.99, 2H) 3.98 (s, 3H) 3.68 (m, 4H) 3.03 (m, 4H); 2.89 (d, J=4.40, 2H) 2.29 (s, 3H), 1.95 (t, J=11.4, 2H), 1.69 (m, 3H), 1.42 (m, 2H). MS (ESI) m/z [M+H]⁺ calcd. for C₂₃H₃₂N₇O₂: 438.2, Found: 438.3; HPLC purity: 210 nm: 100%; 254 nm: 100%.

### Example 24: Synthesis of Exemplary Compound 21

### 3-[4-(5-benzylpyrimidin-2-yl)piperazin-1-yl]-6-(1-methyl-1H-pyrazol-4-yl)imidazo[1,2-a]pyrazine (Compound 21)

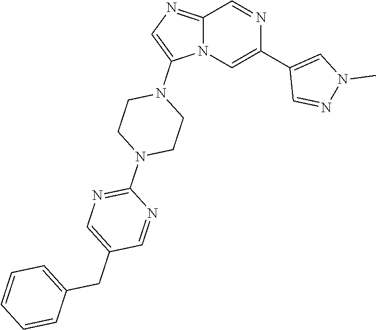 [SMILES: Cn1cc(-c2cn3c(N4CCN(c5ncc(Cc6ccccc6)cn5)CC4)cnc3cn2)cn1]

### Step 1: Preparation of di-tert-butyl 4,4″-[1,2-bis(1H-benzotriazol-1-yl)ethane-1,2-diyl]piperazine-1-carboxylate

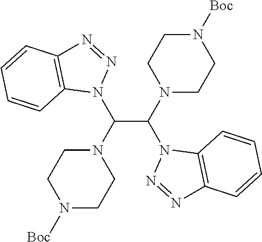 [SMILES: CC(C)(C)OC(=O)N1CCN(C(C(N2CCN(C(=O)OC(C)(C)C)CC2)n2nnc3ccccc32)n2nnc3ccccc32)CC1]

A solution of benzotriazole (2.00 g, 16.8 mmol) and tert-butyl 1-piperazinecarboxylate (3.12 g, 16.8 g) in ethanol (133 mL) was stirred for 20 min at rt. The solution was treated with a 40% solution of aq glyoxal (1.07 mL, 45.1 mmol) and the mixture was stirred at rt for 64 h at which point a white solid had precipitated. The reaction mixture was filtered, the solid washed sequentially with ethanol and diethyl ether followed by drying under vacuum to yield 1.49 g (54%) of di-tert-butyl 4,4″-[1,2-bis(1H-benzotriazol-1-yl)ethane-1,2-diyl]piperazine-1-carboxylate as a white solid. ¹H NMR (400 MHz, CDCl₃): δ (ppm) 7.81 (d, J=8.31 Hz, 2H) 7.52 (d, J=8.31 Hz, 2H) 7.35-7.43 (m, 2H) 7.19-7.26 (m, 2H) 6.97 (s, 2H) 3.35-3.70 (m, 8H) 2.49-3.01 (m, 8H) 1.41 (s, 18H).

### Step 2: Preparation of tert-butyl 4-(6-chloroimidazo[1,2-a]pyrazin-3-yl)piperazine carboxylate

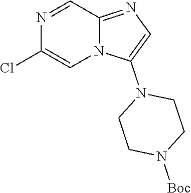 [SMILES: CC(C)(C)OC(=O)N1CCN(c2cnc3cnc(Cl)cn23)CC1]

A mixture of di-tert-butyl 4,4″-[1,2-bis(1H-benzotriazol-1-yl)ethane-1,2-diyl]piperazine-1-carboxylate (0.700, 1.11 mmol), 2-amino-5-chloropyrazine (140 mg, 1.10 mmol) and zinc bromide (250 mg, 1.10 mmol) in 1,2-dichloroethane (18 mL) was heated at 82° C. for 5 h at which time the reaction was determined to be complete by HPLC. The reaction mixture was cooled to RT, filtered through a frit funnel and the solids washed with 20 mL CHCl₃. The filtrate was transferred to a separatory funnel and washed with 25 mL 2N NaOH solution and 20 mL H₂O. The organic phase was dried over Na₂SO₄, filtered and concentrated to a brown oil that was purified by flash chromatography (100 g silica gel, 0-0.5% MeOH/EtOAc) to yield 216 mg (58%) of tert-butyl 4-(6-chloroimidazo[1,2-a]pyrazin-3-yl)piperazine carboxylate as a tan stiff foam. ¹H NMR (400 MHZ, CDCl₃): δ (ppm) 8.83 (d, J=1.34 Hz, 1H) 7.95 (d, J=1.34 Hz, 1H) 7.52 (s, 1H) 3.60-3.72 (m, 4H) 2.97-3.10 (m, 4H) 1.51 (s, 9H); MS (ESI) m/z [M+H]⁺ calcd. for C₁₅H₂₀ClN₅O₂, 338.1; found, 338.1; HPLC purity: 210 nm: 99.4%; 254 nm: 100%.

### Step 3: Preparation of tert-butyl 4-[6-(1-methyl-1H-pyrazol-4-yl)imidazo[1,2-a]pyrazin-3-yl]piperazine-1-carboxylate

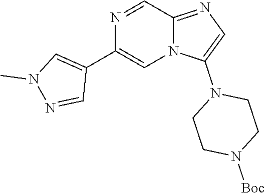 [SMILES: Cn1cc(-c2cn3c(N4CCN(C(=O)OC(C)(C)C)CC4)cnc3cn2)cn1]

A mixture of tert-butyl 4-(6-chloroimidazo[1,2-a]pyrazin-3-yl)piperazine carboxylate (216 mg, 0.64 mmol), 1-methylpyrazole-4-boronic acid pinacol ester (200 mg, 0.96 mmol) and potassium phosphate (271 mg, 1.28 mmol) in 1,4-dioxane (6.5 mL) and H₂O (2.0 mL) was sparged with argon gas for 20 min. The mixture was treated with bis(tri-tert-butylphosphine) palladium (0) (48 mg, 0.094 mmol) and the reaction mixture was heated at 80° C. for 22 h at which point HPLC determined the reaction was complete. The reaction was cooled to rt and concentrated. The residue was treated with 15 mL H₂O and extracted with two 25 mL portions of EtOAc. The combined organic phase was dried over MgSO₄, filtered and concentrated to yield a reddish brown residue that was purified by flash chromatography (80 g silica gel, 4-10% MeOH/CH₂Cl₂) to yield 142 mg (58%) of tert-butyl 4-[6-(1-methyl-1H-pyrazol-4-yl)imidazo[1,2-a]pyrazin-3-yl]piperazine-1-carboxylate as a light brown stiff foam. ¹H NMR (400 MHZ, CDCl₃): δ (ppm) 8.99 (d, J=1.47 Hz, 1H) 7.99 (d, J=1.47 Hz, 1H) 7.92 (s, 1H) 7.88-7.90 (s, 1H) 7.45 (s, 1H) 3.99 (s, 3H) 3.66-3.76 (m, 4H) 2.99-3.12 (m, 4H) 1.52 (m, 9H); MS (ESI) m/z [M+H]⁺ calcd. for C₁₉H₂₅N₇O₂, 384.2; found, 384.2; HPLC purity: 210 nm: 85.9%; 254 nm: 94.1%.

### Step 4: Preparation of 6-(1-methyl-1H-pyrazol-4-yl)-3-piperazin-1-ylimidazo[1,2-a]pyrazine trifluoroacetate salt

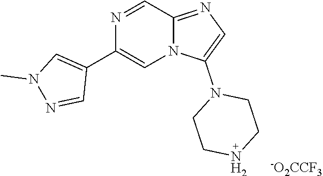

To an ice bath cooled solution of tert-butyl 4-[6-(1-methyl-1H-pyrazol-4-yl)imidazo[1,2-a]pyrazin-3-yl]piperazine-1-carboxylate (142 mg, 0.34 mmol) in 5.7 mL CH₂Cl₂ was added trifluoroacetic acid (1.1 mL, 15 mmol) dropwise. The reaction mixture was stirred at 0° C. for 20 min at which time the ice bath was removed and the reaction was warmed to rt. After 1.5 h, HPLC indicated the starting material had been consumed. The reaction mixture was concentrated to an oil. The oil was dissolved in CH₂Cl₂ and concentrated, repeating the process once to yield 6-(1-methyl-1H-pyrazol-4-yl)-3-piperazin-1-ylimidazo[1,2-a]pyrazine trifluoroacetate salt as a viscous oil that was used in the next step without purification. MS (ESI) m/z [M+H]⁺ calcd. for C₁₄H₁₇N₇, 284.2; found, 284.2; HPLC purity: 210 nm: 100%; 254 nm: 100%.

### Step 5:3-[4-(5-benzylpyrimidin-2-yl)piperazin-1-yl]-6-(1-methyl-1H-pyrazol-4-yl)imidazo[1,2-a]pyrazine (Compound 21)

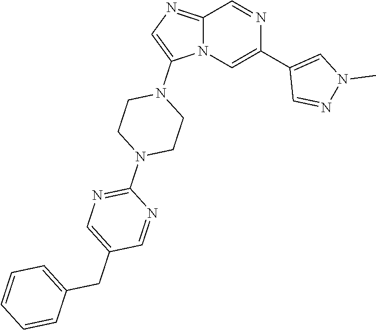 [SMILES: Cn1cc(-c2cn3c(N4CCN(c5ncc(Cc6ccccc6)cn5)CC4)cnc3cn2)cn1]

To a solution of 6-(1-methyl-1H-pyrazol-4-yl)-3-piperazin-1-ylimidazo[1,2-a]pyrazine trifluoroacetate salt (97 mg, 0.29 mmol) in N-methylpyrrolidinone (2.5 mL) was added 5-benzyl-2-chloropyrimidine (72 mg, 0.35 mmol) and K₂CO₃ (163 mg, 1.18 mmol) and the mixture was heated at 110° C. After 5.5 h HPLC indicated the starting material had been consumed and a new product had formed. The reaction mixture was cooled to rt, diluted with 10 mL H₂O and extracted with two 20 mL portions of EtOAc. The combined organic extracts were washed with 10 mL H₂O, dried over MgSO₄, filtered and concentrated to a brown solid that was purified by flash chromatography (35 g silica gel, 30-65% acetone/CH₂Cl₂) to yield a tan solid that was triturated with MTBE to yield 70 mg (53%) of 3-[4-(5-benzylpyrimidin-2-yl)piperazin-1-yl]-6-(1-methyl-1H-pyrazol-4-yl)imidazo[1,2-a]pyrazine (Compound 21) as a light tan solid. ¹H NMR (400 MHZ, CDCl₃): δ (ppm) 9.00 (d, J=1.22 Hz, 1H) 8.23 (s, 2H) 8.05 (d, J=1.22 Hz, 1H) 7.93 (s, 1H) 7.91 (s, 1H) 7.46 (s, 1H) 7.29-7.35 (m, 2H) 7.24 (d, J=7.21 Hz, 1H) 7.19 (d, J=7.21 Hz, 2H) 4.03-4.13 (m, 4H) 3.99 (s, 3H) 3.84 (s, 2H) 3.09-3.25 (m, 4H); MS (ESI) m/z [M+H]⁺ calcd. for C₂₅H₂₅N₉, 452.2; found, 452.3; HPLC purity: 210 nm: 98.1%; 254 nm: 98.2%.

### Example 25: Synthesis of Exemplary Compound 22

### 3-[4-(5-Benzylpyrimidin-2-yl)piperazin-1-yl]-6-(1-methyl-1H-pyrazol-4-yl)pyrazolo[1,5-a]pyrazine (Compound 22)

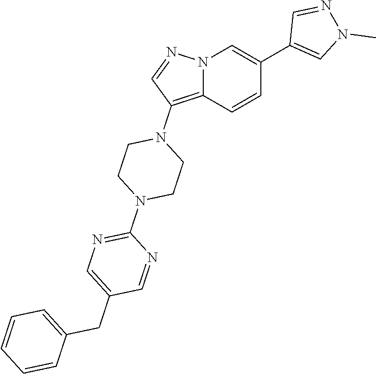 [SMILES: Cn1cc(-c2ccc3c(N4CCN(c5ncc(Cc6ccccc6)cn5)CC4)cnn3c2)cn1]

To a vial containing 3-bromo-6-(1-methyl-1H-pyrazol-4-yl)pyrazolo[1,5-a]pyrazine (Compound S5, 0.200 g, 0.72 mmol) and 5-benzyl-2-piperazin-1-ylpyrimidine (Compound S16, 0.219 g, 0.86 mmol) was added tert-butyl alcohol (8 mL) and 1,4-dioxane (2 mL). The mixture was sparged with argon for 5 min and sodium tert-butoxide (0.104 g, 1.08 mmol) was added. The mixture was again sparged with argon for 5 min and tBuXPhos Pd G1 (49 mg, 0.072 mmol) was added. The mixture was sparged with argon for 5 min and for an additional 2 min with sonication. The vial was placed in a pre-heated block at 55° C. and stirred for 3 h. The mixture was cooled to ambient temperature and satd aq. NaHCO₃added. The mixture was extracted with DCM (3 times) and the combined organics washed with satd aq. NaHCO₃, water, dried (Na₂SO₄), filtered and concentrated. The residue was purified by Biotage chromatography (20 g normal phase cartridge) using a gradient 0-50% acetone in DCM to afford 0.173 g, (53%) of 3-[4-(5-benzylpyrimidin-2-yl)piperazin-1-yl]-6-(1-methyl-1H-pyrazol-4-yl)pyrazolo[1,5-a]pyrazine (Compound 22) as a light yellow solid. ¹H NMR (400 MHZ, CDCl₃): δ (ppm) 9.07 (d, J=1.47 Hz, 1H) 8.39 (d, J=1.34 Hz, 1H) 8.23 (s, 2H) 7.90 (d, J=3.18 Hz, 2H) 7.69 (s, 1H) 7.29-7.37 (m, 2H) 7.16-7.27 (m, 3H) 4.02-4.09 (m, 4H) 3.99 (s, 3H) 3.84 (s, 2H) 3.19-3.29 (m, 4H); MS (ESI) m/z [M+H]⁺ calcd. for C₂₅H₂₅N₉, 451.2; found, 451.3; HPLC purity: 210 nm: 100.0%; 254 nm: 100.0%.

### Example 26: Synthesis of Exemplary Compound 23

### 3-[4-(5-benzylpyrimidin-2-yl)piperazin-1-yl]-7-(1-methyl-1H-pyrazol-4-yl)imidazo[1,2-a]pyridine (Compound 23)

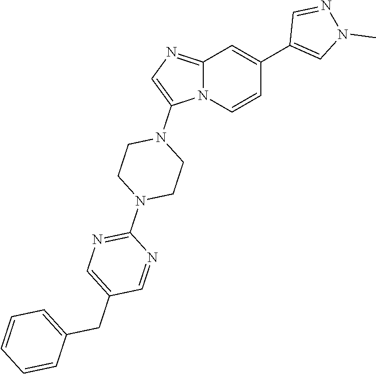 [SMILES: Cn1cc(-c2ccn3c(N4CCN(c5ncc(Cc6ccccc6)cn5)CC4)cnc3c2)cn1]

To a vial containing 5-benzyl-2-piperazin-1-ylpyrimidine (Compound S16, 441 mg, 1.73 mmol) and 3-bromo-7-(1-methyl-1H-pyrazol-4-yl)imidazo[1,2-a]pyridine (Compound S7, 400 mg, 1.45 mmol) was added 1,4-dioxane (7 mL) and tert-butyl alcohol (24 mL). The mixture was sparged with argon for 5 min and sodium tert-butoxide (208 mg, 2.17 mmol) was added. The mixture was sparged again with argon for 5 min and tBuXPhos Pd G1 (0.050 g, 0.072 mmol) was added. The mixture was sparged with argon for 5 min and for an additional 2 min with sonication. After 15 min at ambient temperature, the mixture was placed in a pre-heated block at 55° C. After 9 h, the mixture was cooled and stirred at ambient temperature for 12 h. DCM was added and the organics washed with sat. aq. NaHCO₃, brine, dried (Na₂SO₄), filtered and concentrated. The residue was purified by Biotage chromatography (20 g normal phase cartridge) using a gradient 0-6% methanol (containing 0.1% TEA) in DCM (containing 0.05% TEA) to afford 0.109 g, (20%) of 3-[4-(5-benzylpyrimidin-2-yl)piperazin-1-yl]-7-(1-methyl-1H-pyrazol-4-yl)imidazo[1,2-a]pyridine (Compound 23) as a tan solid. ¹H NMR (400 MHZ, CDCl₃): δ (ppm) 8.48 (s, 1H) 8.23 (s, 2H) 7.73 (s, 1H) 7.76 (s, 1H) 7.63 (s, 1H) 7.55-7.61 (m, 1H) 7.28-7.37 (m, 4H) 7.13-7.27 (m, 4H) 4.00-4.09 (m, 4H) 3.99 (s, 3H) 3.83 (s, 2H) 3.06-3.19 (m, 4H); MS (ESI) m/z [M+H]⁺ calcd. for C₂₆H₂₆N₈, 451.2; found, 451.2; HPLC purity: 210 nm: 100.0%; 254 nm: 100.0%.

### Example 27: Synthesis of Exemplary Compound 24

### 3-[4-(5-benzylpyrimidin-2-yl)piperazin-1-yl]-7-(1-methyl-1H-pyrazol-4-yl)[1,2,4]triazolo[4,3-a]pyridine (Compound 24)

### Step 1: Preparation of 7-(1-methyl-1H-pyrazol-4-yl)[1,2,4]triazolo[4,3-a]pyridine

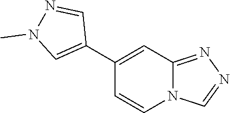

A mixture of 7-bromo[1,2,4]triazolo[4,3-a]pyridine (1.00 g, 5.05 mmol), 1-methylpyrazole-4-boronic acid pinacol ester (1.58 g, 7.57 mmol), potassium carbonate (1.40 g, 10.1 mmol), 1,4-dioxane (9.9 mL), and water (2.0 mL) were combined in a 100 mL round bottom flask and sparged with nitrogen for 230 min. To the reaction was added tetrakis(triphenylphosphine) palladium (0) (0.15 g, 0.13 mmol). The reaction mixture was warmed at 110° C. using an oil bath while stirring under a nitrogen atmosphere. After stirring 24 h at 110° C., added tetrakis(triphenylphosphine) palladium (0) (0.15 g, 0.13 mmol) to the reaction. The reaction was stirred for an additional 4 h at 110° C. then re-dosed with tetrakis(triphenylphosphine) palladium (0) (80 mg, 0.07 mmol). After another hour at 110° C., the heat was removed, and the reaction allowed to stir for 18 h at rt. The reaction was concentrated. The residue was stirred with MeOH and concentrated. The residue was suspended in MeOH and the solids removed by filtration. Silica gel was added to the filtrate and the suspension concentrated. The residue was repurified by ISCO chromatography (80 g cartridge) eluting with a gradient of 20% to 50% IPA in DCM to afford 0.92 g (91%) of 7-(1-methyl-1H-pyrazol-4-yl)[1,2,4]triazolo[4,3-a]pyridine as tan colored solid. MS (ESI) m/z [M+H]⁺ calcd. for C₁₀H₉N₅, 200.1; found, 200.1.

### Step 2: Preparation of 3-bromo-7-(1-methyl-1H-pyrazol-4-yl)[1,2,4]triazolo[4,3-a]pyridine

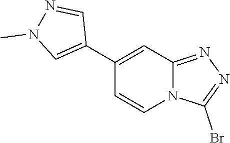 [SMILES: Cn1cc(-c2ccn3c(Br)nnc3c2)cn1]

To a suspension of 7-(1-methyl-1H-pyrazol-4-yl)[1,2,4]triazolo[4,3-a]pyridine (376 mg, 1.89 mmol) in DMF (5.0 mL) was added N-bromosuccinimide (396 mg, 2.08 mmol). The reaction was stirred at rt under a nitrogen atmosphere. After stirring 3 h at rt, added N-bromosuccinimide (34 mg, 0.19 mmol) and continued to stir at rt. After stirring for another hour, the reaction dosed a third time with N-Bromosuccinimide (34 mg, 0.19 mmol). After stirring another hour, the reaction was quenched with a 1:1 mixture of saturated aqueous NaHCO₃ and saturated aqueous Na₂S₂O₃ (10 mL). The reaction was diluted with DCM (10 mL) and stirred for overnight at rt. The layers of the quenched reaction were separated, and the aqueous phase was washed with 20% trifluoroethanol in DCM (5×10 mL). The organic layers were combined and concentrated. The residue was azeotroped several times with MeOH then dissolved in MeOH. Silica gel (5 g) was added to the solution and the suspension concentrated. The residue was purified by ISCO chromatography (24 g cartridge) eluting with a gradient of 0 to 10% IPA in DCM to afford 383 mg (73%) of 3-bromo-7-(1-methyl-1H-pyrazol-4-yl)[1,2,4]triazolo[4,3-a]pyridine as a white solid. m/z [M+H]⁺ calcd. for C₁₀H₈BrN₅, 278.0; found, 278.0.

### Step 3: Preparation of 3-[4-(5-benzylpyrimidin-2-yl)piperazin-1-yl]-7-(1-methyl-1H-pyrazol-4-yl)[1,2,4]triazolo[4,3-a]pyridine (Compound 24)

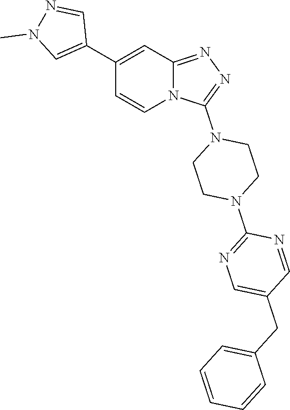 [SMILES: Cn1cc(-c2ccn3c(N4CCN(c5ncc(Cc6ccccc6)cn5)CC4)nnc3c2)cn1]

A suspension of 3-bromo-7-(1-methyl-1H-pyrazol-4-yl)[1,2,4]triazolo[4,3-a]pyridine (100 mg, 0.4 mmol), 5-benzyl-2-piperazin-1-ylpyrimidine (Compound $16, 110 mg, 4.3 mmol), tert-butyl alcohol (2.7 mL) and 1,4-dioxane (0.75 mL) in a 20 mL vial was sparged with argon for 5 min. Added sodium tert-butoxide (42 mg, 0.43 mmol) to the reaction and sparged with argon for 10 min. Then added tBuXPhos Pd G1 (12 mg, 0.02 mmol) to the reaction and sparged with argon for 5 min. Sparged with argon an additional 3 min while subjecting the reaction to ultrasound. The reaction was placed in an oil bath at 55° C. After stirring for 18 h at 55° C., the reaction was cooled to rt and diluted and stirred with saturated aqueous NaHCO₃ (10 mL) and EtOAc (10 mL). The two layers were separated, and the aqueous layer washed with a mixture of 1:1 THF:EtOAc (2×10 mL) The organic layers were combined and concentrated. The residue was dissolved in MeOH. Added silica gel (2 g) to the solution. The suspension was concentrated and the residue purified by ISCO chromatography (12 g cartridge) eluting first with a gradient of 0 to 100% acetone in DCM and then with 50% IPA in DCM to afford 37 mg (20%) of 3-[4-(5-benzylpyrimidin-2-yl)piperazin-1-yl]-7-(1-methyl-1H-pyrazol-4-yl)[1,2,4]triazolo[4,3-a]pyridine (Compound 24) as a yellow colored solid: ¹H NMR (400 MHZ, CDCl₃): δ (ppm) 8.23 (s, 2H) 7.81-7.87 (m, 2H) 7.72 (s, 1H) 7.66 (t, J=1.10 Hz, 1H) 7.28-7.35 (m, 2H) 7.16-7.26 (m, 3H) 6.92 (dd, J=7.21, 1.47 Hz, 1H) 4.03-4.09 (m, 4H) 3.99 (s, 3H) 3.83 (s, 2H) 3.36-3.43 (m, 4H); MS (ESI) m/z [M+H]⁺ calcd. for C₂₅H₂₅N₉, 452.2; found, 452.3; HPLC purity: 210 nm: 98.1%; 254 nm: 100.0%.

### Example 28: Synthesis of Exemplary Compound 25

### (4-fluorophenyl)(2-{4-[6-(1-methyl-1H-pyrazol-4-yl)pyrazolo[1,5-a]pyridin-3-yl]piperazin-1-yl}pyrimidin-5-yl)methanone (Compound 25)

### Step 1: Preparation of ethyl 2-[4-(tert-butoxycarbonyl)piperazin-1-yl]pyrimidine-5-carboxylate

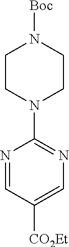 [SMILES: CCOC(=O)c1cnc(N2CCN(C(=O)OC(C)(C)C)CC2)nc1]

To a solution of ethyl 2-chloropyrimidine-5-carboxylate (5.336 g, 28.60 mmol) and tert-butyl 1-piperazinecarboxylate (5.859 g, 31.46 mmol) in 1,4-dioxane (50 mL) was added N,N-diisopropylethylamine (12.4 mL, 71.50 mmol). After 3 h, the volatiles were removed under reduced pressure. The residue was dissolved in ethyl acetate and washed with satd aq. NaHCO₃, brine, dried (Na₂SO₄), filtered and concentrated to provide 9.42 g (98%) of 2-[4-(tert-butoxycarbonyl)piperazin-1-yl]pyrimidine-5-carboxylate as a white solid that was used without further purification. ¹H NMR (400 MHZ, CDCl₃): δ (ppm) 8.85 (s, 2H) 4.35 (q, J=7.09 Hz, 2H) 3.88-3.98 (m, 4H) 3.45-3.58 (m, 4H) 1.50 (s, 9H) 1.38 (t, J=7.15 Hz, 3H); MS (ESI) m/z [M+H]⁺ calcd. for C₁₆H₂₄N₄O₄, 337.4; found, 337.2; HPLC purity: 210 nm: 100.0%; 254 nm: 100.0%.

### Step 2: Preparation of 2-[4-(tert-butoxycarbonyl)piperazin-1-yl]pyrimidine-5-carboxylic acid

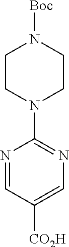 [SMILES: CC(C)(C)OC(=O)N1CCN(c2ncc(C(=O)O)cn2)CC1]

To a solution of ethyl 2-[4-(tert-butoxycarbonyl)piperazin-1-yl]pyrimidine-5-carboxylate (9.42 g, 28.00 mmol) in THF (70 mL) and MeOH (50 mL) was added 1 N sodium hydroxide (42.0 mL, 42.0 mmol). The solution was heated to 70° C. for 2 h. The solution was cooled to ambient temperature and 1 N HCl (43.0 ml) was added. The mixture was acidified to pH of approximately 5 with 0.25 N HCl and extracted three times with ethyl acetate. The combined organics were washed with water, dried (Na₂SO₄), filtered and concentrated to provide 8.28 g (96%) of 2-[4-(tert-butoxycarbonyl)piperazin-1-yl]pyrimidine-5-carboxylic acid as a white solid that was used without further purification. ¹H NMR (400 MHZ, CDCl₃): δ (ppm) 8.84 (s, 2H) 3.80-4.01 (m, 4H) 3.43-3.61 (m, 4H) 1.47 (s, 9H); MS (ESI) m/z [M+H]⁺ calcd. for C₁₄H₂₀N₄O₄, 309.2; found, 309.2; HPLC purity: 210 nm: 100.0%; 254 nm: 100.0%.

### Step 3: Preparation of tert-butyl 4-{5-[methoxy(methyl)carbamoyl]pyrimidin-2-yl}piperazine-1-carboxylate

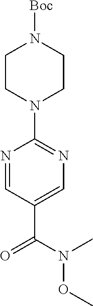 [SMILES: CON(C)C(=O)c1cnc(N2CCN(C(=O)OC(C)(C)C)CC2)nc1]

To a cooled (0-5° C.) solution of 2-[4-(tert-butoxycarbonyl)piperazin-1-yl]pyrimidine-5-carboxylic acid (8.28 g, 26.8 mmol) in DCM (100 mL) and DMF (30 mL) was added 1-hydroxybenzotriazole (12% water w/w, 4.691 g, 27.08 mmol) followed by N,O-dimethylhydroxylamine hydrochloride (2.641 g, 27.08 mmol) and DIEA (4.717 mL, 27.08 mmol). To this solution was added EDC (5.191 g, 27.08 mmol). The mixture was stirred 10 min at 0-5° C., and the ice bath removed. After 16 h at ambient temperature, the mixture was diluted with DCM and the organics washed with satd aq. NaHCO₃, water, 0.1 N HCl, and brine, dried (Na₂SO₄), filtered and concentrated to provide 8.23 g (87%) of tert-butyl 4-{5-[methoxy(methyl)carbamoyl]pyrimidin-2-yl}piperazine-1-carboxylate as a white solid that was used without further purification. ¹H NMR (400 MHZ, CDCl₃): δ (ppm) 8.83 (s, 2H) 3.88-3.95 (m, 4H) 3.65 (s, 3H) 3.49-3.58 (m, 4H) 3.38 (s, 3H) 1.51 (s, 9H); MS (ESI) m/z [M+H]⁺ calcd. for C₁₆H₂₅N₅O₄, 352.2; found, 352.3; HPLC purity: 210 nm: 97.3%; 254 nm: 96.6%.

### Step 4: Preparation of tert-butyl 4-[5-(4-fluorobenzoyl)pyrimidin-2-yl]piperazine-1-carboxylate

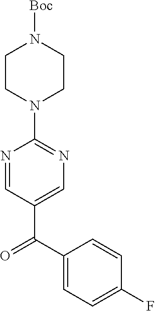 [SMILES: CC(C)(C)OC(=O)N1CCN(c2ncc(C(=O)c3ccc(F)cc3)cn2)CC1]

To a cooled (ca. −10° C.) solution of tert-butyl 4-{5-[methoxy(methyl)carbamoyl]pyrimidin-2-yl}piperazine-1-carboxylate (7.92 g, 22.5 mmol) in THF (48 mL) was slowly added 4-fluorophenylmagnesium bromide (1 M in THF, 50.71 mL, 50.71 mmol) over 30 min. After a total of 1 h, the ice bath was removed, and the mixture stirred an additional 1 h at ambient temperature. The mixture was cooled in an ice bath and quenched by the slow addition of 1 N HCl (50 ml). The pH was further adjusted to ca. 4 with 0.1 HCl and extracted 3 times with ethyl acetate. The combined organics were washed with 0.1 N HCl, water, brine, dried (Na₂SO₄), filtered and concentrated. The residue was purified by Biotage chromatography (330 g normal phase cartridge) eluting with a gradient of 0-5% MeOH in DCM to afford 6.95 g (83%) of tert-butyl 4-[5-(4-fluorobenzoyl)pyrimidin-2-yl]piperazine-1-carboxylate as a white solid. ¹H NMR (400 MHZ, CDCl₃): δ (ppm) 8.78 (s, 2H) 7.75-7.85 (m, 2H) 7.15-7.24 (m, 2H) 3.92-4.03 (m, 4H) 3.48-3.61 (m, 4H) 1.52 (s, 9H); MS (ESI) m/z [M+H]⁺ calcd. for C₂₀H₂₃FN₅O₄, 387.2; found, 387.2; HPLC purity: 210 nm: 100.0%; 254 nm: 100.0%.

### Step 5: Preparation of (4-fluorophenyl)(2-piperazin-1-ylpyrimidin-5-yl)methanone

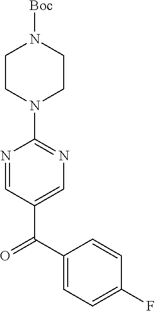 [SMILES: CC(C)(C)OC(=O)N1CCN(c2ncc(C(=O)c3ccc(F)cc3)cn2)CC1]

To a cooled 0-5° C. solution of tert-butyl 4-[5-(4-fluorobenzoyl)pyrimidin-2-yl]piperazine-1-carboxylate (2.500 g, 6.470 mmol) in DCM (62 mL) was added TFA (31 mL). After 90 min at 0-5° C. the volatiles were removed at reduced pressure and the residue concentrated from toluene and DCM (2×). The residue was diluted with DCM and satd aq. NaHCO₃(pH ca. 8-9). The aqueous layer was extracted twice with DCM and combined organics washed with satd aq. NaHCO₃, water, dried (Na₂SO₄), filtered and concentrated to provide 1.73 g (93%) of (4-fluorophenyl)(2-piperazin-1-ylpyrimidin-5-yl)methanone as a pale yellow solid that was used without further purification. ¹H NMR (400 MHZ, CDCl₃): δ (ppm) 8.77 (s, 2H) 7.71-7.86 (m, 2H) 7.14-7.24 (m, 2H) 3.88-4.06 (m, 4H) 2.89-3.06 (m, 4H); MS (ESI) m/z [M+H]⁺ calcd. for C₁₅H₁₅FN₄O, 287.1; found, 287.1; HPLC purity: 210 nm: 100.0%; 254 nm: 100.0%.

### Step 6: (4-fluorophenyl)(2-{4-[6-(1-methyl-1H-pyrazol-4-yl)pyrazolo[1,5-a]pyridin-3-yl]piperazin-1-yl}pyrimidin-5-yl)methanone (Compound 25)

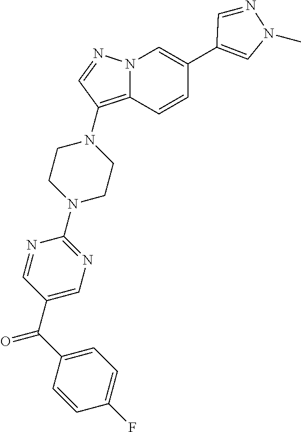 [SMILES: Cn1cc(-c2ccc3c(N4CCN(c5ncc(C(=O)c6ccc(F)cc6)cn5)CC4)cnn3c2)cn1]

To a flame dried flask containing 3-bromo-6-(1-methyl-1H-pyrazol-4-yl)pyrazolo[1,5-a]pyridine (Compound S9, 1.00 g, 3.61 mmol) and (4-fluorophenyl)(2-piperazin-1-ylpyrimidin-5-yl)methanone (1.24 g, 4.33 mmol) was added tert-butyl alcohol (30 mL) and 1,4-dioxane (15 mL). The solution was sparged with argon for 5 min. To the solution was added sodium tert-butoxide (0.520 g, 5.41 mmol). The mixture was sparged 5 min with argon and tBuXPhos Pd G1 (248 mg, 0.36 mmol) was added. The mixture sparged 5 min with argon and then an additional 2 min with sonication. The mixture heated at 55° C. for 3 h, cooled and diluted with ethyl acetate. The organics were washed with sat. aq. NaHCO₃, brine, dried (Na₂SO₄), filtered and concentrated. The residue was purified by Biotage chromatography (80 g normal phase cartridge) eluting with a gradient of 0-50% acetone in DCM to afford 0.92 g (53%) of (4-fluorophenyl)(2-{4-[6-(1-methyl-1H-pyrazol-4-yl)pyrazolo[1,5-a]pyridin-3-yl]piperazin-1-yl}pyrimidin-5-yl)methanone (Compound 25) as a pale yellow solid. ¹H NMR (400 MHZ, CDCl₃): δ (ppm) 8.81 (s, 2H) 8.49 (s, 1H) 7.79-7.86 (m, 2H) 7.76 (d, J=7.34 Hz, 2H) 7.64 (s, 1H) 7.54-7.61 (m, 1H) 7.15-7.24 (m, 3H) 4.18-4.26 (m, 4H) 3.99 (s, 3H) 3.11-3.23 (m, 4H); MS (ESI) m/z [M+H]⁺ calcd. for C₂₆H₂₃FN₈O, 483.2; found, 483.3; HPLC purity: 210 nm: 100.0%; 254 nm: 100.0%.

### Example 29: Synthesis of Exemplary Compound 26

### (4-fluorophenyl)(2-{4-[6-(1-methyl-1H-pyrazol-4-yl)pyrazolo[1,5-a]pyridin-3-yl]piperazin-1-yl}pyrimidin-5-yl)methanol (Compound 26)

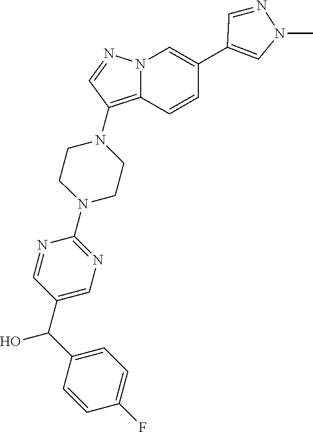 [SMILES: Cn1cc(-c2ccc3c(N4CCN(c5ncc(C(O)c6ccc(F)cc6)cn5)CC4)cnn3c2)cn1]

To a cooled (0-5° C.) mixture of (4-fluorophenyl)(2-{4-[6-(1-methyl-1H-pyrazol-4-yl)pyrazolo[1,5-a]pyridin-3-yl]piperazin-1-yl}pyrimidin-5-yl)methanone (Compound 25, 0.600 g, 1.24 mmol) in methanol (6 mL) was added sodium borohydride (0.094 g, 2.49 mmol). After 10 min, the ice bath was removed, and mixture stirred at rt for 1 h. THF (10 mL) and additional MeOH (10 mL) were added to aid in dissolution. The reaction was warmed to 40° C., and monitored by HPLC. Additional sodium borohydride was added portion wise to effect complete conversion (total of 5 extra equivalents). The mixture was cooled in an ice bath and quenched by the addition of saturated aq. NH₄Cl. The mixture was extracted with DCM (3 times) and the combined organics washed with aq. NaHCO₃, water, dried (Na₂SO₄), filtered and concentrated. The residue was purified by Biotage chromatography (80 g normal phase cartridge) eluting with a gradient of 0-5% methanol in DCM to afford 0.41 g (68%) of (4-fluorophenyl)(2-{4-[6-(1-methyl-1H-pyrazol-4-yl)pyrazolo[1,5-a]pyridin-3-yl]piperazin-1-yl}pyrimidin-5-yl)methanol (Compound 26) as a yellow solid. ¹H NMR (400 MHZ, CDCl₃): δ (ppm) 8.48 (s, 1H) 8.31 (s, 2H) 7.72 (s, 1H) 7.76 (s, 1H) 7.63 (s, 1H) 7.57 (d, J=9.17 Hz, 1H) 7.33-7.43 (m, 2H) 7.16 (dd, J=9.11, 1.41 Hz, 1H) 7.03-7.13 (m, 2H) 5.77 (d, J=3.18 Hz, 1H) 4.01-4.09 (m, 4H) 3.99 (s, 3H) 3.06-3.19 (m, 4H) 2.30 (d, J=3.55 Hz, 1H); MS (ESI) m/z [M+H]⁺ calcd. for C₂₆H₂₅FN₈O, 485.2; found, 485.3; HPLC purity: 210 nm: 100.0%; 254 nm: 100.0%.

### Example 30: Synthesis of Exemplary Compound 27

### tert-butyl 4-[6-(1-methyl-1H-pyrazol-4-yl)pyrazolo[1,5-a]pyridine-3-yl]-5-oxo-1,4-diazepane-1-carboxylate (Compound 27)

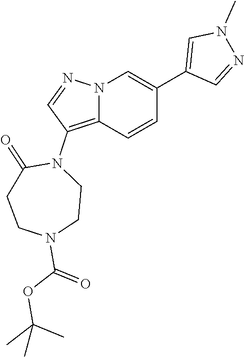 [SMILES: Cn1cc(-c2ccc3c(N4CCN(C(=O)OC(C)(C)C)CCC4=O)cnn3c2)cn1]

To a solution of 3-bromo-6-(1-methyl-1H-pyrazolo-4-yl)pyrazolo[1,5-a]pyridine (Compound S9, 500 mg, 1.80 mmol) and tert-butyl 5-oxo-1,4 diazepane-1-carboxylate (425 mg, 1.98 mmol) in 1,4-dioxane (9.8 mL) was treated with N,N′-dimethyl-1,2-ethanediamine (0.077 mL, 0.72 mmol), copper (I) iodide (68 mg, 0.36 mmol) and potassium phosphate (1.15 g, 5.41 mmol) and the reaction mixture was heated at 110° C. for 22 h at which point HPLC indicated the starting material had been consumed. The reaction mixture was cooled to rt, diluted with 40 mL EtOAc and filtered through a pad of Celite, washing the pad with an additional 40 mL EtOAc. The filtrate was concentrated to a light brown solid that was purified by flash chromatography (100 g silica gel; 30-60% acetone/CH₂Cl₂) to afford 648 mg (87%) of tert-butyl 4-[6-(1-methyl-1H-pyrazol-4-yl)pyrazolo[1,5-a]pyridine-3-yl]-5-oxo-1,4-diazepane-1-carboxylate as a light yellow solid which contained ˜30% of the diazepane starting material. A 72 mg sample of the material was subjected to Combiflash reverse phase chromatography (15 g RediSep C-18 Gold column) eluting with a gradient of 10% CH₃CN/H₂O to 100% CH₃CN to afford 23 mg of tert-butyl 4-[6-(1-methyl-1H-pyrazol-4-yl)pyrazolo[1,5-a]pyridine-3-yl]-5-oxo-1,4-diazepane-1-carboxylate (Compound 27) as a white solid. ¹H NMR (400 MHZ, CDCl₃): δ (ppm) 8.50 (s, 1H) 7.84 (s, 1H) 7.74 (s, 1H) 7.62 (s, 1H) 7.36 (dd, J=9.17, 0.73 Hz, 1H) 7.23-7.29 (m, 1H) 3.98 (s, 3H) 3.85-3.95 (m, 2H) 3.80 (d, J=7.95 Hz, 4H) 2.84-3.00 (m, 2H) 1.51 (s, 9H); MS (ESI) m/z [M+H]⁺ calcd. for C₂₁H₂₈N₆O₂: 411.2; found, 411.3; HPLC purity: 210 nm: 98.3%; 254 nm: 100%.

### Example 31: Synthesis of Exemplary Compound 28

### tert-butyl 4-[6-(1-methyl-1H-pyrazol-4-yl)pyrazolo[1,5-a]pyridine-3-yl]-3-oxopiperazine-1-carboxylate (Compound 28)

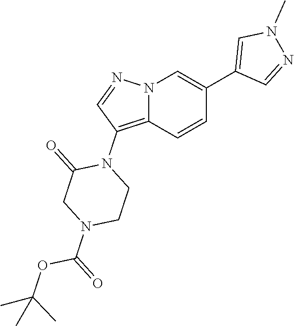 [SMILES: Cn1cc(-c2ccc3c(N4CCN(C(=O)OC(C)(C)C)CC4=O)cnn3c2)cn1]

To a solution of 3-bromo-6-(1-methyl-1H-pyrazolo-4-yl)pyrazolo[1,5-a]pyridine (Compound S9, 75 mg, 0.27 mmol) and tert-butyl 3-oxopiperazine-1-carboxylate (59 mg, 0.30 mmol) in 1,4-dioxane (1.5 mL) was treated with N,N′-dimethyl-1,2-ethanediamine (0.012 mL, 0.11 mmol), copper (I) iodide (10 mg, 0.054 mmol) and potassium phosphate (172 mg, 0.81 mmol) and the reaction mixture was heated at 110° C. for 41 h at which point HPLC indicated the starting material had been consumed. The reaction mixture was cooled to rt, diluted with 10 mL EtOAc and filtered through a pad of Celite, washing the pad with an additional 20 mL EtOAc. The filtrate was concentrated to a brown solid that was purified by flash chromatography (60 g silica gel; 10-50% acetone/CH₂Cl₂) to afford 63 mg of a light yellow glass which was triturated with Et₂O followed by lyophilization from aqueous CH₃CN to yield 49 mg (46%) of tert-butyl 4-[6-(1-methyl-1H-pyrazol-4-yl)pyrazolo[1,5-a]pyridine-3-yl]-3-oxopiperazine-1-carboxylate (Compound 28) as a light gray solid. ¹H NMR (400 MHZ, CDCl₃): δ (ppm) 8.52 (s, 1H) 7.91 (s, 1H) 7.75 (d, J=0.49 Hz, 1H) 7.63 (s, 1H) 7.42 (dd, J=9.17, 0.73 Hz, 1H) 7.27-7.32 (m, 1H) 4.33 (s, 2H) 3.98 (s, 3H) 3.84-3.90 (m, 2H) 3.78-3.84 (m, 2H) 1.53 (s, 9H); MS (ESI) m/z [M+H]⁺ calcd. for C₂₁H₂₈N₆O₂: 397.2; found, 397.3; HPLC purity: 210 nm: 99.6%; 254 nm: 99.5%.

### Example 32: Synthesis of Exemplary Compound 29

### 3-[4-(5-benzylpyrimidin-2-yl)piperazin-1-yl]-6-[1-(2-methoxyethyl)-1H-pyrazol-4-yl]pyrazolo[1,5-a]pyridine (Compound 29)

### Step 1: Preparation of 6-[1-(2-methoxyethyl)-1H-pyrazol-4-yl]pyrazolo[1,5-a]pyridine

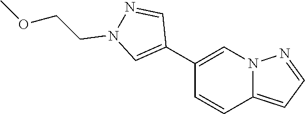 [SMILES: COCCn1cc(-c2ccc3ccnn3c2)cn1]

A mixture of 6-bromopyrazolo[1,5-a]pyridine (1.00 g, 5.08 mmol), 1-(2-methoxyethyl)-4-(4,4,5,5-tetramethyl-1,3,2-dioxaborolan-2-yl)-1H-pyrazole (1.60 g, 6.34 mmol), 1,4-dioxane (10.0 mL), and water (2.0 mL) were combined in a 100 mL round bottom flask and sparged with nitrogen for 20 min. To the reaction was added tetrakis(triphenylphosphine) palladium (0) (147 mg, 127 mmol) and potassium carbonate (1.40 g, 10.2 mmol). The reaction mixture was sparged with nitrogen for 5 min then warmed to 110° C. using an oil bath while stirring under a nitrogen atmosphere. After stirring 1.5 h at 110° C., added tetrakis(triphenylphosphine) palladium (0) (80 mg, 69 mmol) to the reaction. The reaction was stirred for 18 h at 110° C. then cooled to rt. The reaction was partitioned between H₂O (35 mL) and DCM (35 mL). The aqueous layer was washed with DCM (3×25 mL). The DCM layers were combined, washed with brine, dried with Na₂SO₄ and concentrated. The residue was purified by ISCO chromatography (40 g cartridge) eluting with a gradient of DCM to 50% Acetone in DCM. The product obtained was repurified by ISCO chromatography (40 g cartridge) eluting with a gradient of 0 to 100% EtOAc in hexane to afford 1.00 g (81%) of 6-[1-(2-methoxyethyl)-1H-pyrazol-4-yl]pyrazolo[1,5-a]pyridine as dark oil. MS (ESI) m/z [M+H]⁺ calcd. for C₁₃H₁₄N₄O, 243.1; found, 243.1.

### Step 2: Preparation of 3-bromo-6-[1-(2-methoxyethyl)-1H-pyrazol-4-yl]pyrazolo[1,5-a]pyridine

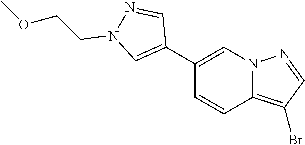 [SMILES: COCCn1cc(-c2ccc3c(Br)cnn3c2)cn1]

To a solution of 6-[1-(2-methoxyethyl)-1H-pyrazol-4-yl]pyrazolo[1,5-a]pyridine (995 mg, 4.11 mmol) in DMF (11 mL) was added N-bromosuccinimide (810 mg, 4.55 mmol). The reaction was stirred at rt under a nitrogen atmosphere. After 30 min, the reaction was quenched with a 1:1 mixture of saturated aqueous NaHCO₃ and saturated aqueous Na₂S203 (24 mL). The reaction was diluted with DCM (24 mL) and stirred for 10 min. The layers of the quenched reaction were separated, and the aqueous phase was washed with DCM (3×12 mL). The DCM layers were combined, dried (Na₂SO₄) and filtered. Silica gel (7 g) was added to the filtrate and the suspension concentrated. The residue was purified by ISCO chromatography (40 g normal phase cartridge) eluting with a gradient of 0 to 100% ethyl acetate in hexanes. The purified product was dissolved in EtOAc (80 mL) and washed with water (6×10 mL) then brine (1×10 mL) to remove succinimide. The EtOAc layer was dried (Na₂SO₄) and concentrated to afford 972 mg (74%) of 3-bromo-6-[1-(2-methoxyethyl)-1H-pyrazol-4-yl]pyrazolo[1,5-a]pyridine as a tan colored solid. MS (ESI) m/z [M+H]⁺ calcd. for C₁₃H₁₃BrN₄O, 321.0; found, 321.1.

### Step 3: Preparation of 3-[4-(5-benzylpyrimidin-2-yl)piperazin-1-yl]-6-[1-(2-methoxyethyl)-1H-pyrazol-4-yl]pyrazolo[1,5-a]pyridine (Compound 29)

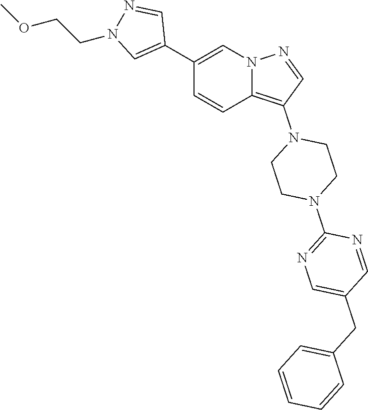 [SMILES: COCCn1cc(-c2ccc3c(N4CCN(c5ncc(Cc6ccccc6)cn5)CC4)cnn3c2)cn1]

A suspension of 3-bromo-6-[1-(2-methoxyethyl)-1H-pyrazol-4-yl]pyrazolo[1,5-a]pyridine (824 mg, 2.56 mmol), 5-benzyl-2-piperazin-1-ylpyrimidine (Compound S16, 783 mg, 3.08 mmol), tert-butyl alcohol (19 mL) and 1,4-dioxane (5 mL) in a 100 mL round bottom flask was sparged with argon for 5 min. Added sodium tert-butoxide (296 mg, 3.08 mmol) to the reaction and sparged with argon for 5 min. Then added tBuXPhos Pd G1 (88 mg, 0.13 mmol) to the reaction and sparged with argon for 5 min. Sparged with argon an additional 2 min while subjecting the reaction to ultrasound. The reaction was placed in an oil bath at 55° C. After stirring for 18 h at 55° C. the reaction was cooled to rt and diluted and stirred with saturated aqueous NaHCO₃ (40 mL) and EtOAc (20 mL). The two layers were separated, and the aqueous layer diluted with water (30 mL) and then washed with EtOAc (3×20 mL). The EtOAc layers were combined, dried (Na₂SO₄) and concentrated. The residue was purified by ISCO chromatography (120 g normal phase cartridge) eluting with a gradient of 0 to 70% acetone in DCM to afford 337 mg (27%) of 3-[4-(5-benzylpyrimidin-2-yl)piperazin-1-yl]-6-[1-(2-methoxyethyl)-1H-pyrazol-4-yl]pyrazolo[1,5-a]pyridine (Compound 29) as a tan colored solid. ¹H NMR (400 MHZ, CDCl₃): δ (ppm) 8.49 (s, 1H) 8.21 (s, 2H) 7.78 (s, 1H) 7.74 (s, 1H) 7.72 (s, 1H) 7.56 (dd, J=9.23, 0.67 Hz, 1H) 7.26-7.34 (m, 2H) 7.12-7.25 (m, 4H) 4.35 (t, J=5.14 Hz, 2H) 3.95-4.07 (m, 4H) 3.76-3.86 (m, 4H) 3.38 (s, 3H) 3.08-3.18 (m, 4H); MS (ESI) m/z [M+H]⁺ calcd. for C₂₈H₃₀N₈O, 495.2; found, 495.4; HPLC purity: 210 nm: 98.2%; 254 nm: 99.2%.

### Example 33: Synthesis of Exemplary Compound 30

### 2-phenylethyl-4-[6-(1-methyl-1H-pyrazol-4-yl)pyrazolo[1,5-a]pyridin-3-yl]piperazine-1-carboxylate (Compound 30)

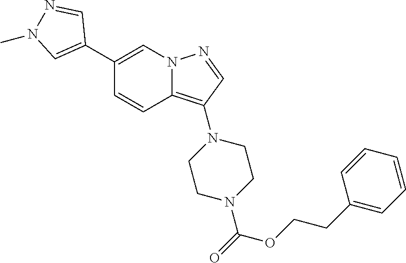 [SMILES: Cn1cc(-c2ccc3c(N4CCN(C(=O)OCCc5ccccc5)CC4)cnn3c2)cn1]

To a vial containing 2-phenylethan-1-ol (28 mg, 0.23 mmol) was added DCM (1.5 mL) followed by Et₃N (89 mg, 0.89 mmol) then 4-nitrophenyl chlorocarbonate (46 mg, 23 mmol). The reaction was stirred at rt. After 45 min. added 6-(1-methyl-1H-pyrazol-4-yl)-3-piperazin-1-ylpyrazola [1,5-a]pyridine dihydrochloride (Compound S19, 70 mg, 0.22 mmol). The reaction was stirred 18 h at rt. The reaction was cooled to 0° C., and quenched with water (4 mL). The reaction was extracted with DCM (3×4 mL). The combined DCM layers were dried (Na₂SO₄) filtered and concentrated. The residue was purified by ISCO chromatography (12 g normal phase cartridge) eluting with EtOAc. The solids obtained were stirred with Et₂O and concentrated. This was repeated three more times. The reside was then stirred with MeOH and concentrated. This was repeated four times and the solid dried in vacuo at 40° C. to afford 53 mg, (57%) of 2-phenylethyl4-[6-(1-methyl-1H-pyrazol-4-yl)pyrazolo[1,5-a]pyridin-3-yl]piperazine-1-carboxylate (Compound 30). ¹H NMR (400 MHz, CDCl₃): δ (ppm) 8.47 (s, 1H) 7.75 (s, 1H) 7.69 (s, 1H) 7.61 (s, 1H) 7.50 (d, J=9.17 Hz, 1H) 7.29-7.35 (m, 2H) 7.22-7.28 (m, 3H) 7.15 (dd, J=9.11, 1.41 Hz, 1H) 4.36 (t, J=6.97 Hz, 2H) 3.98 (s, 3H) 3.57-3.72 (m, 4H) 2.93-3.07 (m, 6H); MS (ESI) m/z [M+H]⁺ calcd. for C₂₄H₂₆N₂O₂, 431.2; found, 431.3; HPLC purity: 210 nm: 98.7%; 254 nm: 98.8%.

### Example 34: Synthesis of Exemplary Compound 31

### isopropyl 4-[6-(1-methyl-1H-pyrazol-4-yl)pyrazolo[1,5-a]pyridin-3-yl]piperazine-1-carboxylate (Compound 31)

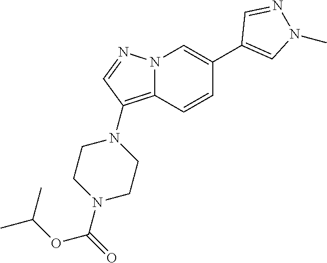 [SMILES: CC(C)OC(=O)N1CCN(c2cnn3cc(-c4cnn(C)c4)ccc23)CC1]

Compound 31 was prepared as described for the preparation of 1-ethylpropyl 4-[6-(1-methyl-1H-pyrazol-4-yl)pyrazolo[1,5-a]pyridin-3-yl]piperazine-1-carboxylate (Example 19, Compound 16) using isopropanol (47 μL, 0.62 mmol) to afford 112 mg (76%) of isopropyl 4-[6-(1-methyl-1H-pyrazol-4-yl)pyrazolo[1,5-a]pyridin-3-yl]piperazine-1-carboxylate (Compound 31) as a beige solid. ¹H NMR (400 MHZ, CDCl₃): δ (ppm) 8.47 (s, 1H) 7.75 (s, 1H) 7.71 (s, 1H), 7.61 (s, 1H) 7.52 (d, J=9.41, 1H) 7.15 (dd, J=9.17, 1.34 Hz, 1H) 4.97 (spt, J=6.26, 1H) 3.98 (s, 3H) 3.68 (m, 4H) 3.03 (m, 4H) 1.29 (d, J=6.24, 6H); MS (ESI) m/z [M+H]⁺ calcd. for C₁₉H₂₄N₆O₂: 369.2, Found: 369.3; HPLC purity: 210 nm: 100.0%; 254 nm: 100.0%.

### Example 35: Synthesis of Exemplary Compound 32

### cyclobutyl 4-[6-(1-methyl-1H-pyrazol-4-yl)pyrazolo[1,5-a]pyridin-3-yl]piperazine-1-carboxylate (Compound 32)

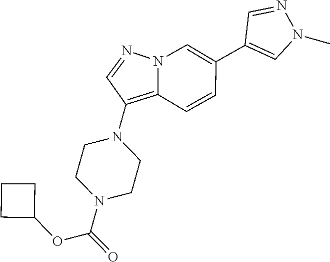 [SMILES: Cn1cc(-c2ccc3c(N4CCN(C(=O)OC5CCC5)CC4)cnn3c2)cn1]

Compound 32 was prepared as described for the preparation of 1-ethylpropyl 4-[6-(1-methyl-1H-pyrazol-4-yl)pyrazolo[1,5-a]pyridin-3-yl]piperazine-1-carboxylate (Example 19, Compound 16) using cyclobutanol (49 μL, 0.62 mmol) to afford 119 mg (78%) of cyclobutyl 4-[6-(1-methyl-1H-pyrazol-4-yl)pyrazolo[1,5-a]pyridin-3-yl]piperazine-1-carboxylate (Compound 32) as a beige solid. ¹H NMR (400 MHZ, CDCl₃): δ (ppm) 8.47 (s, 1H) 7.75 (s, 1H) 7.71 (s, 1H), 7.61 (s, 1H) 7.52 (d, J=6.36, 1H) 7.16 (d, J=9.17 Hz, 1H) 4.99 (m, 1H) 3.98 (s, 3H) 3.69 (m, 4H) 3.04 (m, 4H) 2.38 (m, 2H), 2.10 (m, 2H), 1.79 (m, 1H), 1.63 (m, 1H); MS (ESI) m/z [M+H]⁺ calcd. for C₂₀H₂₄N₆O₂: 381.2, Found: 381.2; HPLC purity: 210 nm: 100.0%; 254 nm: 100.0%.

### Example 36: Synthesis of Exemplary Compound 33

### (R)-1-phenylethyl 4-(6-(1-methyl-1H-pyrazol-4-yl)pyrazolo[1,5-a]pyridin-3-yl)piperazine-1-carboxylate (Compound 33)

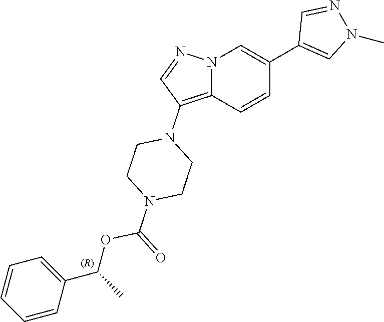

Compound 33 was prepared as described for the preparation of 1-ethylpropyl 4-[6-(1-methyl-1H-pyrazol-4-yl)pyrazolo[1,5-a]pyridin-3-yl]piperazine-1-carboxylate (Example 19, Compound 16) using (R)-1-phenylethan-1-ol (76 μL, 0.62 mmol) to afford 136 mg (76%) of (R)-1-phenylethyl 4-(6-(1-methyl-1H-pyrazol-4-yl)pyrazolo[1,5-a]pyridin-3-yl)piperazine-1-carboxylate (Compound 33) as a beige solid. ¹H NMR (400 MHz, CDCl₃): δ (ppm) 8.46 (s, 1H) 7.74 (s, 1H) 7.70 (s, 1H), 7.61 (s, 1H) 7.50 (d, J=9.05, 1H) 7.38 (s, 2H) 7.37 (d, J=0.86) 7.36 to 7.28 (m, 1H) 7.15 (dd, J=9.17, 1.47 Hz, 1H) 5.87 (q, J=6.56, 1H), 3.98 (s, 3H) 3.72 (br. s., 4H) 3.04 (br.s., 4H) 1.59 (d, J=6.60, 3H); MS (ESI) m/z [M+H]⁺ calcd. for C₂₄H₂₆N₆O₂: 430.2, Found: 431.3; HPLC purity: 210 nm: 100.0%; 254 nm: 100.0%.

### Example 37: Synthesis of Exemplary Compound 34

### (S)-1-phenylethyl 4-(6-(1-methyl-1H-pyrazol-4-yl)pyrazolo[1,5-a]pyridin-3-yl)piperazine-1-carboxylate (Compound 34)

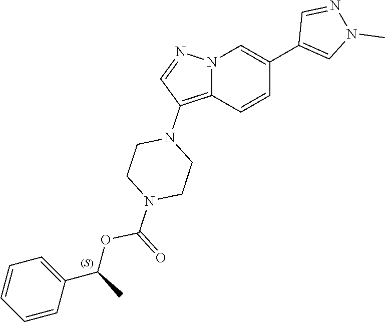

Compound 34 was prepared as described for the preparation of 1-ethylpropyl 4-[6-(1-methyl-1H-pyrazol-4-yl)pyrazolo[1,5-a]pyridin-3-yl]piperazine-1-carboxylate (Example 19, Compound 16) using(S)-1-phenylethan-1-ol (76 μL, 0.62 mmol) to afford 132 mg (76%) of (S)-1-phenylethyl 4-(6-(1-methyl-1H-pyrazol-4-yl)pyrazolo[1,5-a]pyridin-3-yl)piperazine-1-carboxylate (Compound 34) as a beige solid. ¹H NMR (400 MHz, CDCl₃): δ (ppm) 8.47 (s, 1H) 7.75 (s, 1H) 7.70 (s, 1H), 7.61 (s, 1H) 7.51 (d, J=8.68, 1H) 7.38 (s, 2H), 7.37 (s, 2H) 7.36 to 7.28 (m, 1H) 7.15 (dd, J=9.17, 1.10 Hz, 1H) 5.87 (q, J=6.56, 1H), 3.98 (s, 3H) 3.73 (br. s., 4H) 3.04 (br.s., 4H) 1.59 (d, J=6.60, 3H); MS (ESI) m/z [M+H]⁺ calcd. for C₂₄H₂₆N₆O₂: 430.2, Found: 431.3; HPLC purity: 210 nm: 100.0%; 254 nm: 100.0%.

### Example 38: Synthesis of Exemplary Compound 35

### 1-(4-fluorophenyl)-1-(2-{4-[6-(1-methyl-1H-pyrazol-4-yl)pyrazolo[1,5-a]pyridin-3-yl]piperazin-1-yl}pyrimidin-5-yl)ethanamine (Compound 35)

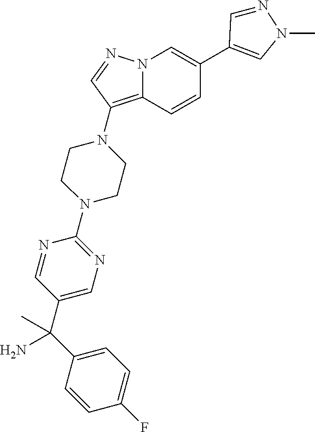 [SMILES: Cn1cc(-c2ccc3c(N4CCN(c5ncc(C(C)(N)c6ccc(F)cc6)cn5)CC4)cnn3c2)cn1]

### Step 1. Preparation of tert-butyl 4-{5-[(Z)-{[(S)-tert-butylsulfinyl]imino}(4-fluorophenyl)methyl]pyrimidin-2-yl}piperazine-1-carboxylate

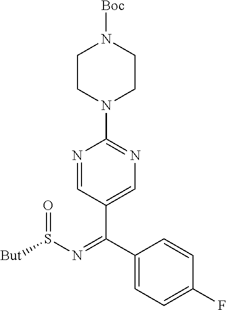

To a flame dried flask containing tert-butyl 4-[5-(4-fluorobenzoyl)pyrimidin-2-yl]piperazine-1-carboxylate (Compound 25; Step 4, 1.100 g, 2.85 mmol) and (R)-(+)-t-butylsulfinamide (0.518 g, 4.27 mmol, previously dried by azeotropic distillation with toluene) was added THF (10 mL) followed by lithium hydroxide (0.017 g, 0.71 mmol) and titanium tetraisopropoxide (1.260 mL, 4.27 mmol). The mixture was heated to 60° C. for 3 h, cooled to ambient temperature and diluted with satd aq NaHCO₃. The mixture was extracted 3 times with ethyl acetate. The combined organics were washed with brine, dried (Na₂SO₄), filtered and concentrated. The residue was recrystallized from ethyl acetate/hexanes to provide 1.136 g (81%) of tert-butyl 4-{5-[(Z)-{[(S)-tert-butylsulfinyl]imino}(4-fluorophenyl)methyl]pyrimidin-2-yl}piperazine-1-carboxylate as a pale yellow solid. ¹H NMR (400 MHZ, DMSO-d₆): δ (ppm) 8.45 (br. s., 2H) 7.68 (br. s., 1H) 7.49 (br. s., 1H) 7.29-7.41 (m, 2H) 3.74-3.96 (m, 4H) 3.44 (br. s., 4H) 1.43 (s, 9H) 1.20 (br. s., 9H); MS (ESI) m/z [M+H]⁺ calcd. for C₂₄H₃₃FN₅O₃S, 490.2; found, 490.3; HPLC purity: 210 nm: 100.0%; 254 nm: 100.0%.

### Step 2. Preparation of tert-butyl 4-{5-[1-{[(S)-tert-butylsulfinyl]amino}-1-(4-fluorophenyl)ethyl]pyrimidin-2-yl}piperazine-1-carboxylate

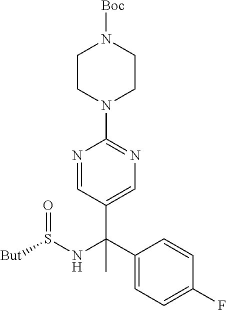

To a cooled (−10° C.) solution of tert-butyl 4-{5-[(Z)-{[(S)-tert-butylsulfinyl]imino}(4-fluorophenyl)methyl]pyrimidin-2-yl}piperazine-1-carboxylate (1.10 g, 2.25 mmol) in THF (20 mL) was slowly added methylmagnesium chloride (1.87 mL of a 3.0 M solution in THF, 5.62 mmol) maintaining the internal temperature below-5° C. After 90 min, the reaction was quenched by the slow addition of MeOH. After 5 min, water was added followed by satd aqueous NH₄Cl. The mixture was allowed to warm to ambient temperature and diluted with water. The pH was adjusted to approx. 8 with NaHCO₃ and the aqueous mixture extracted 3 times with ethyl acetate. The combined organics were washed with brine, dried (Na₂SO₄), filtered and concentrated. The residue was purified by Biotage chromatography (120 g normal phase cartridge) eluting with a gradient of 0-80% ethyl acetate in hexanes to afford 1.04 g (91%) of tert-butyl 4-{5-[1-{[(S)-tert-butylsulfinyl]amino}-1-(4-fluorophenyl)ethyl]pyrimidin-2-yl}piperazine-1-carboxylate as a tan solid. ¹H NMR appears as an approx. equal mixture of diastereomers (400 MHZ, CDCl₃): δ (ppm) 8.32 (s, 1H) 8.27 (s, 1H) 7.37 (overlapping m, 2H) 6.95-7.11 (overlapping m, 2H) 3.71-3.88 (m, 5H) 3.43-3.58 (m, 4H) 2.06 (s, 3H) 1.50 (s, 9H) 1.27 and 1.25 (s, 9H); MS (ESI) m/z [M+H]⁺ calcd. for C₂₅H₃₇FN₅O₃S, 506.2; found, 506.4; HPLC purity: 210 nm: 100.0%; 254 nm: 100.0%.

### Step 3. Preparation of 1-(4-fluorophenyl)-1-(2-piperazin-1-ylpyrimidin-5-yl)ethanamine

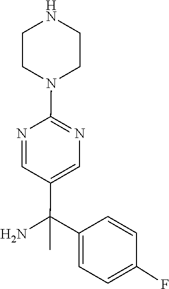 [SMILES: CC(N)(c1ccc(F)cc1)c1cnc(N2CCNCC2)nc1]

To a cooled (0-5° C.) solution of tert-butyl 4-{5-[1-{[(S)-tert-butylsulfinyl]amino}-1-(4-fluorophenyl)ethyl]pyrimidin-2-yl}piperazine-1-carboxylate (1.049 g, 2.07 mmol) in DCM (0.8 mL) was added hydrogen chloride (4 M in 1,4-dioxane, 3.112 mL, 12.45 mmol). After 10 min, the ice bath was removed, and the mixture heated to 40° C. After 1 h, the mixture was cooled, and the volatiles removed under reduced pressure. The residue was concentrated twice each from ethyl acetate and MTBE. The resulting solid was suspended in ethyl acetate and washed with saturated aqueous NaHCO₃ and brine, dried (Na₂SO₄), filtered and concentrated to afford 0.575 g (92%) of 1-(4-fluorophenyl)-1-(2-piperazin-1-ylpyrimidin-5-yl)ethanamine as a white solid. ¹H NMR (400 MHZ, CDCl₃): δ (ppm) 8.31 (s, 2H) 7.32-7.44 (m, 2H) 6.98 (t, J=8.68 Hz, 2H) 3.71-3.82 (m, 4H) 2.85-2.97 (m, 4H) 1.80 (s, 3H); MS (ESI) m/z [M+H]⁺ calcd. for C₁₆H₂₁FN₅, 302.2; found, 302.2; HPLC purity: 210 nm: 100.0%; 254 nm: 100.0%.

### Step 4. Preparation of 1-(4-fluorophenyl)-1-(2-{4-[6-(1-methyl-1H-pyrazol-4-yl)pyrazolo[1,5-a]pyridin-3-yl]piperazin-1-yl}pyrimidin-5-yl)ethanamine (Compound 35)

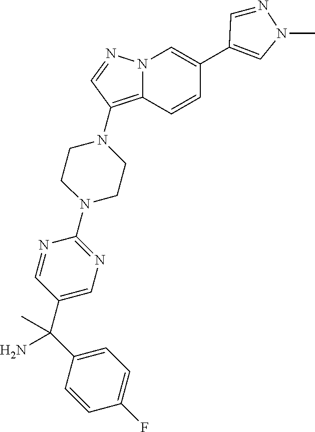 [SMILES: Cn1cc(-c2ccc3c(N4CCN(c5ncc(C(C)(N)c6ccc(F)cc6)cn5)CC4)cnn3c2)cn1]

To a solution of 1-(4-fluorophenyl)-1-(2-piperazin-1-ylpyrimidin-5-yl)ethanamine (0.125 g, 0.42 mmol) and 3-bromo-6-(1-methyl-1H-pyrazol-4-yl)pyrazolo[1,5-a]pyridine (0.138 g, 0.50 mmol) in tert-butyl alcohol (5 mL) and 1,4-dioxane (2 mL) was added sodium tert-butoxide (0.060 g, 0.62 mmol). The mixture was sparged with argon for 5 min and tBuXPhos Pd G1 (0.029 g, 0.041 mmol) was added. The mixture was sparged with argon for 5 min and then for 3 min with sonication. The mixture was heated at 55° C. for 4.5 h. Additional tBuXPhos Pd G1 (ca. 8 mg) and sodium tert-butoxide (ca. 10 mg) were added and the mixture heated at 45° C. for 16 h. The reaction was cooled to ambient temperature and saturated aqueous NaHCO₃added. The mixture was extracted 3 times with DCM and the combined organics washed with saturated aqueous NaHCO₃ and brine, dried (Na₂SO₄), filtered and concentrated. The residue was purified by Biotage chromatography (20 g normal phase cartridge) eluting with a gradient of 0-20% acetone (containing 0.05% TEA) in DCM to afford 0.050 g (24%) of 1-(4-fluorophenyl)-1-(2-{4-[6-(1-methyl-1H-pyrazol-4-yl)pyrazolo[1,5-a]pyridin-3-yl]piperazin-1-yl}pyrimidin-5-yl)ethanamine (Compound 35) as a grey solid. ¹H NMR (400 MHZ, CDCl₃): δ (ppm) 8.48 (s, 1H) 8.38 (s, 2H) 7.73 (s, 1H) 7.76 (s, 1H) 7.63 (s, 1H) 7.57 (d, J=9.17 Hz, 1H) 7.41 (dd, J=8.80, 5.26 Hz, 2H) 7.16 (d, J=9.29 Hz, 1H) 7.02 (t, J=8.68 Hz, 2H) 4.01-4.09 (m, 4H) 3.99 (s, 3H) 3.06-3.20 (m, 4H) 1.85 (s, 3H); MS (ESI) m/z [M+H]⁺ calcd. for C₂₇H₂₈FN₉, 498.2; found, 498.4; HPLC purity: 210 nm: 100.0%; 254 nm: 100.0%.

### Example 39: Synthesis of Exemplary Compound 36

### 3-[4-(5-benzylpyrimidin-2-yl)pyrrolidin-1-yl]-6-(1-methyl-1H-pyrazol-4-yl)pyrazolo[1,5a]pyridine (Compound 36)

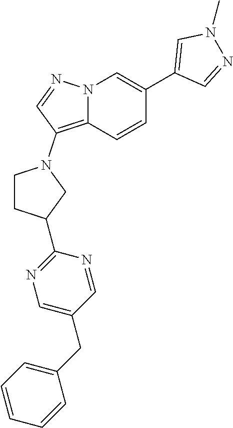 [SMILES: Cn1cc(-c2ccc3c(N4CCC(c5ncc(Cc6ccccc6)cn5)C4)cnn3c2)cn1]

### Step 1. Preparation of tert-butyl 3-(5-benzylpyrimidin-2-yl)-2,5-dihydro-1H-pyrrole-1-carboxylate

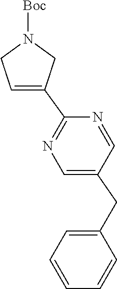 [SMILES: CC(C)(C)OC(=O)N1CC=C(c2ncc(Cc3ccccc3)cn2)C1]

A mixture of tert-butyl 3-(4,4,5,5-tetramethyl-1,3,2-dioxaborolan-2-yl)-2,5-dihydro-1H-pyrrole-1-carboxylate (500.0 mg, 1.69 mmol), 5-benzyl-2-chloropyrimidine (289 mg, 1.41 mmol) and potassium phosphate (599 mg, 2.82 mmol) in 1,4-dioxane (14.3 mL) and H₂O (5.1 mL) was sparged with argon gas for 20 min. The mixture was treated with bis(tri-tert-butylphosphine) palladium (0) (108 mg, 0.21 mmol) and the reaction mixture was heated at 80° C. for 4 h at which time the starting material had been consumed. The reaction mixture was cooled to rt, diluted with 40 mL H₂O and extracted with two 25 mL portions of EtOAc. The combined organic phase was washed with 25 mL H₂O, dried over MgSO₄, filtered and concentrated to yield a brown oil that was purified by flash chromatography (100 g silica gel, 20-30% EtOAc/hex) to yield 420 mg (88%) of tert-butyl 3-(5-benzylpyrimidin-2-yl)-2,5-dihydro-1H-pyrrole-1-carboxylate as a white solid: 1H NMR (400 MHZ, CDCl₃): δ (ppm) 8.56 (s, 2H) 7.31-7.39 (m, 2H) 7.24-7.31 (m, 1H) 7.20 (d, J=6.97 Hz, 2H) 6.83-6.95 (m, 1H) 4.53-4.69 (m, 2H) 4.33-4.49 (m, 2H) 3.89-4.03 (m, 2H) 1.53 (s, 9H); MS (ESI) m/z [M+H]⁺ calcd. for C₂₀H₂₄N₃O₂: 338.2; found, 338.3; HPLC purity 210 nm: 98.3%; 254 nm: 100.0%.

### Step 2. Preparation of tert-butyl 3-(5-benzylpyrimidin-2-yl)pyrrolidine-1-carboxylate

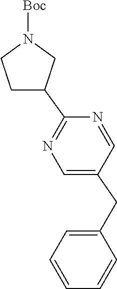 [SMILES: CC(C)(C)OC(=O)N1CCC(c2ncc(Cc3ccccc3)cn2)C1]

To a solution of tert-butyl 3-(5-benzylpyrimidin-2-yl)-2,5-dihydro-1H-pyrrole-1-carboxylate (437 mg, 1.30 mmol) in EtOAc (20 mL) was added 10% palladium on carbon (100 mg; 50% water wet). The reaction flask was evacuated and filled with hydrogen gas three times and allowed to stir under an atmosphere of hydrogen. After 19 h, the starting material was consumed as evidenced by HPLC. The reaction mixture was filtered through a pad of celite, washing the pad with an additional 40 mL of EtOAc. The filtrate was concentrated to a viscous oil that was purified by flash chromatography (60 g silica gel, 25-40% EtOAc/hex) to yield 377 mg (86%) of tert-butyl 3-(5-benzylpyrimidin-2-yl)pyrrolidine-1-carboxylate as a colorless viscous oil. ¹H NMR (400 MHZ, CDCl₃): δ (ppm) 8.52 (s, 2H) 7.30-7.39 (m, 2H) 7.22-7.30 (m, 1H) 7.19 (d, J=7.09 Hz, 2H) 3.95 (s, 2H) 3.83 (m, 1H) 3.53-3.73 (m, 3H) 3.43 (m, 1H) 2.18-2.43 (m, 2H) 1.47 (s, 9H); MS (ESI) m/z [M+H]⁺ calcd. for C₂₀H₂₅N₃O₂: 340.2; found, 340.3; HPLC purity: 210 nm: 100.0%; 254 nm 100.0%.

### Step 3. Preparation of 5-benzyl-2-pyrrolidin-3-ylpyrimidine

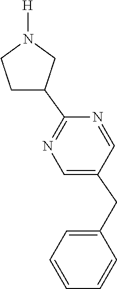 [SMILES: c1ccc(Cc2cnc(C3CCNC3)nc2)cc1]

A solution of tert-butyl 3-(5-benzylpyrimidin-2-yl)pyrrolidine-1-carboxylate (377 mg, 1.11 mmol) in CH₂Cl₂ (9.0 mL) was cooled at 0° C. in an ice bath and treated dropwise with TFA (1.0 mL 13 mmol). The reaction mixture was stirred for 30 min at 0° C. then warmed to RT. After 3 h the reaction was determined to be complete and was concentrated. The resultant oil was taken up in 10 mL CH₂Cl₂ and concentrated again. The crude TFA salt was taken up in 20 mL CH₂Cl₂ and washed with 20 mL sat NaHCO₃ solution. The organic phase was dried over Na₂SO₄, filtered, and concentrated to a yield 211 mg (79%) of 5-benzyl-2-pyrrolidin-3-ylpyrimidine as a light pink viscous oil that was suitable for use in the next step. ¹H NMR (400 MHz, CD₃OD): δ (ppm) 8.58 (s, 2H) 7.27-7.36 (m, 2H) 7.19-7.27 (m, 3H) 3.99 (s, 2H) 3.56-3.69 (m, 1H) 3.32-3.38 (m, 1H) 3.23-3.30 (m, 1H) 3.13-3.23 (m, 1H) 3.08 (dt, J=11.19, 7.37 Hz, 1H) 2.24-2.38 (m, 1H) 2.07-2.24 (m, 1H); MS (ESI) m/z [M+H]⁺ calcd. for C₁₅H₁₇N₃: 240.2; found, 240.1; HPLC purity: 210 nm: 100.0%; 254 nm: 100.0%.

### Step 4. Preparation of 3-[3-(5-benzylpyrimidin-2-yl)pyrrolidin-1-yl]-6-(1-methyl-1H-pyrazol-4-yl)pyrazolo[1,5-a]pyridine (Compound 36)

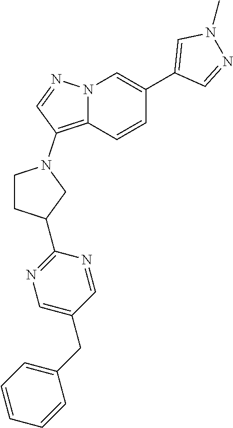 [SMILES: Cn1cc(-c2ccc3c(N4CCC(c5ncc(Cc6ccccc6)cn5)C4)cnn3c2)cn1]

A solution of 3-bromo-6-(1-methyl-1H-pyrazol-4-yl)pyrazolo[1,5-a]pyridine (Compound S9, 204 mg, 0.735 mmol), 5-benzyl-2-pyrrolidin-3-ylpyrimidine (211 mg, 0.882 mmol) in t-butanol (5.5 mL) and 1,4-dioxane (1.5 mL) was sparged for 20 min with argon. The solution was treated with tBuXPhos Pd G1 (76 mg, 0.110 mmol) and sodium tert-butoxide (106 mg, 1.10 mmol) and the reaction mixture was sparged with argon for 10 min. The reaction mixture was heated at 55° C. for 2 h at which point the starting material had been consumed. The reaction was cooled to rt, diluted with 30 mL EtOAc and washed with two 20 mL portions of H₂O. The solution was dried over MgSO₄, filtered, and concentrated to a brown viscous oil that was purified by flash chromatography (80 g silica gel, 20-25% acetone/CH₂Cl₂ followed by 5-10% CH₃OH/CH₂Cl₂) to yield 52 mg of impure product. The material was purified by preparative TLC eluting with 2% CH₃OH/CH₂Cl₂ to yield 27 mg (11%) of 3-[3-(5-benzylpyrimidin-2-yl)pyrrolidine-1-yl]-6-(1-methyl-1H-pyrazol-4-yl)pyrazolo[1,5-a]pyridine (Compound 36) as a yellow glass. ¹H NMR (400 MHz, CDCl₃): δ (ppm) 8.55 (s, 2H) 8.37 (s, 1H) 7.72 (s, 1H) 7.59-7.64 (m, 1H) 7.58 (s, 1H) 7.53 (s, 1H) 7.30-7.38 (m, 2H) 7.22-7.28 (m, 1H) 7.19 (d, J=7.21 Hz, 2H) 6.95 (dd, J=9.29, 1.22 Hz, 1H) 3.97 (s, 5H) 3.85-3.93 (m, 1H) 3.80 (t, J=8.19 Hz, 1H) 3.69-3.76 (m, 1H) 3.54 (t, J=6.91 Hz, 2H) 2.42-2.59 (m, 2H); MS (ESI) m/z [M+H]⁺ calcd. for C₂₆H₂₅N₇: 436.2; found, 436.4; HPLC purity: 210 nm: 95.4%; 254 nm: 95.8%.

### Example 40: Synthesis of Exemplary Compound 37

### 33-[4-(5-benzylpyrimidin-2-yl)-1,4-diazepan-1-yl]-6-(1-methyl-1H-pyrazol-4-yl)pyrazolo[1,5-a]pyridine (Compound 37)

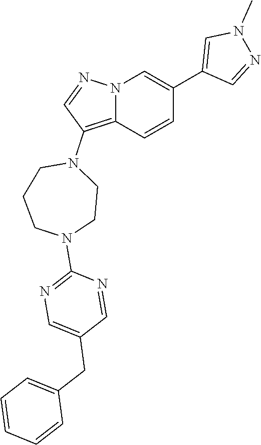 [SMILES: Cn1cc(-c2ccc3c(N4CCCN(c5ncc(Cc6ccccc6)cn5)CC4)cnn3c2)cn1]

### Step 1. Preparation of [6-(1-methyl-1H-pyrazol-4-yl)pyrazolo[1,5-a]pyridine-3-yl]-1,4-diazepan-1-ium trifluoroacetate

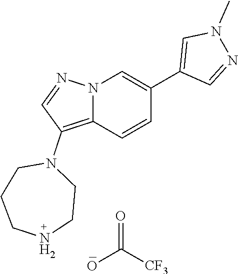

A solution of tert-butyl 4-[6-(1-methyl-1H-pyrazol-4-yl)pyrazolo[1,5-a]pyridine-3-yl]-1,4-diazepane-1-carboxylate (Compound 40, 213 mg, 0.537 mmol) in methylene chloride (11 mL) was cooled at 0° C. in an ice bath and treated dropwise with TFA (2.3 mL). The reaction mixture was stirred for 30 min at 0° C. at which point the ice bath was removed and the reaction was warmed to RT. After 1.5 h the reaction was found to be complete, and the reaction mixture was concentrated. The crude product was taken up in 5 mL methylene chloride and concentrated, repeating the process one additional time. The crude TFA salt was placed under high vacuum to yield [6-(1-methyl-1H-pyrazol-4-yl)pyrazolo[1,5-a]pyridine-3-yl]-1,4-diazepan-1-ium trifluoroacetate as a brown oil that and used in the next step without purification assuming a 100% yield. ¹H NMR (400 MHZ, CDCl₃): δ (ppm) 8.41 (d, J=5.26 Hz, 1H) 7.59-7.75 (m, 3H) 7.38-7.54 (m, 1H) 7.06-7.21 (m, 1H) 3.90 (d, J=5.99 Hz, 3H) 3.46 (br. s., 2H) 3.24-3.39 (m, 6H) 2.15 (d, J=5.01 Hz, 2H); MS (ESI) m/z [M+H]⁺ calcd. for C₁₆H₂₀N₆: 297.2; found, 297.3; HPLC purity: 210 nm: 100.0%; 254 nm: 98.2%.

### Step 2. Preparation of 3-[4-(5-benzylpyrimidin-2-yl)-1,4-diazepan-1-yl]-6-(1-methyl-1H-pyrazol-4-yl)pyrazolo[1,5-a]pyridine (Compound 37)

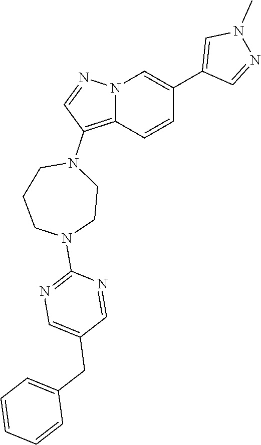 [SMILES: Cn1cc(-c2ccc3c(N4CCCN(c5ncc(Cc6ccccc6)cn5)CC4)cnn3c2)cn1]

To a solution of [6-(1-methyl-1H-pyrazol-4-yl)pyrazolo[1,5-a]pyridine-3-yl]-1,4-diazepan-1-ium trifluoroacetate (220 mg, 0.54 mmol) and 5-benzyl-2-chloropyrimidine (115 mg, 0.56 mmol) in NMP (4.0 mL) was added K₂CO₃ (370 mg, 2.68 mmol) and the reaction mixture was heated at 110° C. After 8 h the reaction was found to be complete. The reaction mixture was cooled to rt, diluted with 25 mL EtOAc and washed with two 15 mL portions of H₂O. The organic phase was dried over MgSO₄, filtered, and concentrated to yield a brown oil that was purified by flash chromatography (80 g silica gel, 10-40% acetone/CH₂Cl₂) to afford a stiff foam. Trituration of the material with Et₂O followed by filtration yielded 172 mg (69%) of 3-[4-(5-benzylpyrimidin-2-yl)-1,4-diazepan-1-yl]-6-(1-methyl-1H-pyrazol-4-yl)pyrazolo[1,5-a]pyridine (Compound 37) as a light yellow solid: ¹H NMR (400 MHZ, CDCl₃): δ (ppm) 8.40 (s, 1H) 8.18 (s, 2H) 7.73 (s, 1H) 7.62 (s, 1H) 7.59 (s, 1H) 7.47 (d, J=9.17 Hz, 1H) 7.28-7.35 (m, 2H) 7.15-7.25 (m, 3H) 7.04 (d, J=9.17 Hz, 1H) 4.00-4.09 (m, 2H) 3.97 (s, 3H) 3.89 (t, J=6.24 Hz, 2H) 3.80 (s, 2H) 3.42-3.49 (m, 2H) 3.26-3.38 (m, 2H) 2.10 (quin, J=5.90 Hz, 2H); MS (ESI) m/z [M+H]⁺ calcd. for C₂₇H₂₈N₈: 465.3; found, 465.4; HPLC purity: 210 nm: 97.4%; 254 nm: 97.7%.

### Example 41: Synthesis of Exemplary Compound 38

### 3-[4-(5-benzylpyrimidin-2-yl)piperazin-1-yl]-6-(1-methyl-1H-pyrazol-3-yl)pyrazolo[1,5-a]pyridine (Compound 38)

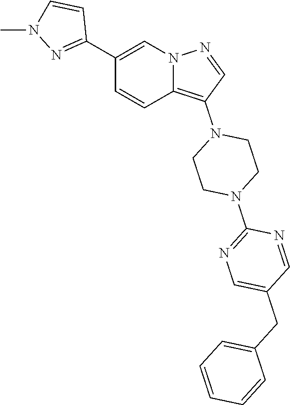 [SMILES: Cn1ccc(-c2ccc3c(N4CCN(c5ncc(Cc6ccccc6)cn5)CC4)cnn3c2)n1]

### Step 1. Preparation of 6-(1-methyl-1H-pyrazol-3-yl)pyrazolo[1,5-a]pyridine

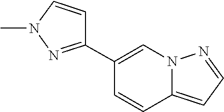

Into a 20 mL vial with rubber septum was added of 6-bromopyrazolo[1,5-a]pyridine (0.12 g, 0.61 mmol), 1-methyl-3-(4,4,5,5-tetramethyl-1,3,2-dioxaborolan-2-yl)-1H-pyrazole (0.16 g, 0.75 mmol), 1,4-dioxane (1.2 mL) and H₂O (0.25 mL). The reaction mixture was sparged with nitrogen for 15 min. Tetrakis(triphenylphosphine) palladium (0) (18 mg, 0.02 mmol) and K₂CO₃ (167 mg, 1.20 mmol) were added to the reaction and the mixture sparged for another 5 min. The reaction was put under a nitrogen atmosphere, sealed, and warmed in an oil bath at 110° C. overnight. The reaction was cooled to rt and partitioned between DCM (10 mL) and H₂O (10 mL). The aqueous layer was washed with DCM (2×5 mL). The combined organic layers were washed with brine and then partially evaporated to reduce the volume of DCM. The residual DCM solution was purified by ISCO chromatography (12 g cartridge) eluting with a gradient of EtOAc in hexanes to afford 94 mg (79%) of 6-(1-methyl-1H-pyrazol-3-yl)pyrazolo[1,5-a]pyridine as a tan solid. MS (ESI) m/z [M+H]⁺ calcd. for C₁₁H₁₀N₄, 199.1; found, 199.1.

### Step 2. Preparation of 3-bromo-6-(1-methyl-1H-pyrazol-3-yl)pyrazolo[1,5-a]pyridine

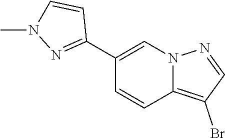 [SMILES: Cn1ccc(-c2ccc3c(Br)cnn3c2)n1]

To a solution of 6-(1-methyl-1H-pyrazol-3-yl)pyrazolo[1,5-a]pyridine (87 mg, 0.44 mmol) dissolved in DMF (1.2 mL) was added N-bromosuccinimide (86 mg, 0.48 mmol). After 30 min the reaction was quenched with a mixture of aq sodium thiosulfate (2 mL) and satd aq NaHCO₃ (2 mL). The quenched reaction was washed with DCM (3×4 mL). The combined DCM layers were dried (Na₂SO₄), filtered and concentrated. The residue was purified by ISCO chromatography (24 g normal phase cartridge) eluting with a gradient of ethyl acetate in hexanes to afford 95 mg (79%) of 3-bromo-6-(1-methyl-1H-pyrazol-3-yl)pyrazolo[1,5-a]pyridine as a white solid. MS (ESI) m/z [M+H]⁺ calcd. for C₁₁H₉BrN₄, 277.0; found, 277.0.

### Step 3. Preparation of 3-[4-(5-benzylpyrimidin-2-yl)piperazin-1-yl]-6-(1-methyl-1H-pyrazol-3-yl)pyrazolo[1,5-a]pyridine (Compound 38)

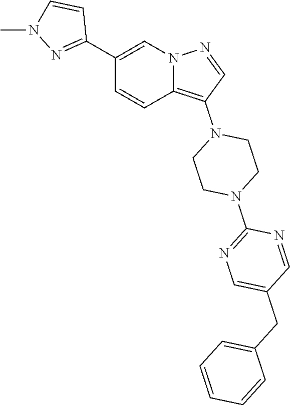 [SMILES: Cn1ccc(-c2ccc3c(N4CCN(c5ncc(Cc6ccccc6)cn5)CC4)cnn3c2)n1]

To a vial containing 3-bromo-6-(1-methyl-1H-pyrazol-3-yl)pyrazolo[1,5-a]pyridine (92 mg, 0.33 mmol) and 5-benzyl-2-piperazin-1-ylpyrimidine (Compound S16, 0.10 g, 0.40 mmol) was added tert-butyl alcohol (2.4 mL) and 1,4-dioxane (0.70 mL). The partially dissolved mixture was sparged with argon for 5 min. To the mixture was added sodium tert-butoxide (38 mg, 0.40 mmol). The mixture was sparged again for 5 min with argon and tBuXPhos Pd G1 (11 mg, 0.02 mmol) added. The reaction mixture was sparged with argon for 5 min and an additional 2 min with sonication. The flask was heated in a 55° C. oil bath for 18 h, then cooled to ambient temperature. The reaction was stirred with a mixture of EtOAc (10 mL) and satd aq NaHCO₃ (10 mL). The two layers were separated. The aq. layer was diluted with water (5 mL) and then washed with EtOAc (3×8 mL). The combined EtOAc layers were dried (Na₂SO₄), filtered and concentrated. The residue was purified by ISCO chromatography (24 g normal phase cartridge) eluting with a gradient of 0-50% acetone in DCM afford 41 mg (28%) of 3-[4-(5-benzylpyrimidin-2-yl)piperazin-1-yl]-6-(1-methyl-1H-pyrazol-3-yl)pyrazolo[1,5-a]pyridine (Compound 38) as a tan solid. ¹H NMR (400 MHZ, CDCl₃): δ (ppm) 8.73 (s, 1H) 8.21 (s, 2H) 7.74 (s, 1H) 7.51-7.61 (m, 2H) 7.41 (d, J=2.20 Hz, 1H) 7.28-7.35 (m, 2H) 7.15-7.25 (m, 3H) 6.52 (d, J=2.32 Hz, 1H) 3.93-4.06 (m, 7H) 3.82 (s, 2H) 3.08-3.19 (m, 4H); MS (ESI) m/z [M+H]⁺ calcd. for C₂₆H₂₆N₈, 451.2; found, 451.3; HPLC purity: 210 nm: 100.0%; 254 nm: 100.0%.

### Example 42: Synthesis of Exemplary Compound 39

### tetrahydro-2H-pyran-4-yl 4-[6-(1-methyl-1H-pyrazol-4-yl)pyrazolo[1,5-a]pyridin-3-yl]piperazine-1-carboxylate (Compound 39)

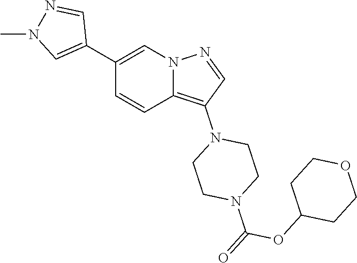 [SMILES: Cn1cc(-c2ccc3c(N4CCN(C(=O)OC5CCOCC5)CC4)cnn3c2)cn1]

To a vial containing tetrahydro-2H-pyran-4-ol (25 mg, 0.25 mmol was added DCM (1.6 mL) followed by Et₃N (96 mg, 0.94 mmol) and 4-nitrophenyl chlorocarbonate (50 mg, 0.25 mmol). After 1 h at rt, 6-(1-methyl-1H-pyrazol-4-yl)-3-piperazin-1yl)pyrazolo[1,5-a]pyridine dihydrochloride (Compound S19, 75 mg, 0.24 mmol) was added to the reaction. After stirring 26 h at rt, the reaction was quenched with water (4 mL). The quenched reaction was extracted with DCM (3×4 mL). The combined DCM layers were dried (Na₂SO₄) filtered and concentrated. The residue was dissolved in a mixture of DCM and MeOH. Added silica gel to the solution and concentrated the suspension under vacuum. The residue was purified by ISCO chromatography (24 g normal phase cartridge) eluting with EtOAc to afford 0.34 mg (35%) of tetrahydro-2H-pyran-4-yl 4-[6-(1-methyl-1H-pyrazol-4-yl)pyrazolo[1,5-a]pyridin-3-yl]piperazine-1-carboxylate (Compound 39) as a tan solid. ¹H NMR (400 MHZ, CDCl₃): δ (ppm) 8.47 (s, 1H) 7.75 (s, 1H) 7.71 (s, 1H) 7.62 (s, 1H) 7.51 (d, J=9.17 Hz, 1H) 7.16 (dd, J=9.17, 1.34 Hz, 1H) 4.93 (tt, J=8.44, 4.10 Hz, 1H) 3.88-4.02 (m, 5H) 3.66-3.75 (m, 4H) 3.59 (ddd, J=11.74, 8.86, 3.00 Hz, 2H) 2.98-3.11 (m, 4H) 1.94-2.05 (m, 2H) 1.72 (dtd, J=13.04, 8.76, 4.03 Hz, 2H); MS (ESI) m/z [M+H]⁺ calcd. for C₂₁H₂₆N₆O₃, 411.2; found, 411.3; HPLC purity: 210 nm: 99.7%; 254 nm: 99.7%.

### Example 43: Synthesis of Exemplary Compound 40

### Tert-butyl 4-[6-(1-methyl-1H-pyrazol-4-yl)pyrazolo[1,5-a]pyridine-3-yl]-1,4-diazepane-1-carboxylate (Compound 40)

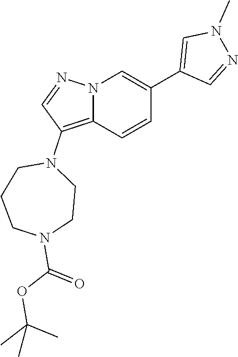 [SMILES: Cn1cc(-c2ccc3c(N4CCCN(C(=O)OC(C)(C)C)CC4)cnn3c2)cn1]

### Step 1. Preparation of tert-butyl 4-[6-(1-methyl-1H-pyrazol-4-yl)pyrazolo[1,5-a]pyridine-3-yl]-5-thioxo-1,4-diazepane-1-carboxylate

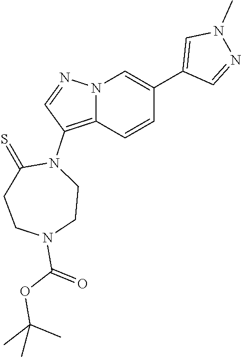 [SMILES: Cn1cc(-c2ccc3c(N4CCN(C(=O)OC(C)(C)C)CCC4=S)cnn3c2)cn1]

To a solution of tert-butyl 4-[6-(1-methyl-1H-pyrazol-4-yl)pyrazolo[1,5-a]pyridine-3-yl]-5-oxo-1,4-diazepane-1-carboxylate (Compound 27, 564 mg, 1.37 mmol) in THF (20 mL) was added 2,4-bis(4-methoxyphenyl)-2,4-dithioxo-1,3,2,4-dithiadiphosphetane (390 mg, 0.96 mmol) and the reaction mixture was stirred at 60° C. After 2 h the reaction was 40% complete, an additional 220 mg of 2,4-bis(4-methoxyphenyl)-2,4-dithioxo-1,3,2,4-dithiadiphosphetane was added and heating was continued for an additional 1.5 h at which point the reaction was determined to be complete. The reaction mixture was cooled, concentrated to ˜1/2 the original volume. The reaction mixture was purified directly by flash chromatography (100 g silica gel, 15-30% acetone/CH₂Cl₂) to afford 471 mg (80%) of tert-butyl 4-[6-(1-methyl-1H-pyrazol-4-yl)pyrazolo[1,5-a]pyridine-3-yl]-5-thioxo-1,4-diazepane-1-carboxylate as a light yellow solid. ¹H NMR (400 MHZ, CDCl₃): δ (ppm) 8.53 (s, 1H) 7.92 (s, 1H) 7.75 (s, 1H) 7.62 (s, 1H) 7.34-7.41 (m, 1H) 7.29-7.34 (m, 1H) 4.11-4.19 (m, 2H) 3.98 (s, 3H) 3.78-3.89 (m, 4H) 3.45-3.56 (m, 2H) 1.51 (s, 9H); MS (ESI) m/z [M+H]⁺ calcd. for C₂₁H₂₇N₆O₂S: 427.2; found, 427.3; HPLC purity: 210 nm: 98.6%; 254 nm: 99.7%.

### Step 2. Preparation of tert-butyl 4-[6-(1-methyl-1H-pyrazol-4-yl)pyrazolo[1,5-a]pyridine-3-yl]-1,4-diazepane-1-carboxylate (Compound 40)

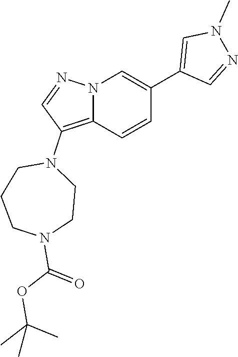 [SMILES: Cn1cc(-c2ccc3c(N4CCCN(C(=O)OC(C)(C)C)CC4)cnn3c2)cn1]

To a slurry of Raney nickel (4.7 g) in THF (5.0 mL) was added a solution of tert-butyl 4-[6-(1-methyl-1H-pyrazol-4-yl)pyrazolo[1,5-a]pyridine-3-yl]-5-thioxo-1,4-diazepane-1-carboxylate (471 mg, 1.10 mmol) in THF (20 mL) and the reaction mixture was stirred at rt. After 1 h the reaction was 90% complete. An additional 0.5 g of Raney nickel was added and stirring was continued for an additional 1.5 h at which point it was determined the starting material had been consumed. The reaction mixture was filtered through a pad of celite, washing the pad with additional THF (75 mL) and the filtrate was concentrated to a yellow glass. The crude product was purified by flash chromatography (100 g silica gel, 20-40% acetone/CH₂Cl₂) to afford 269 mg (61%) of tert-butyl 4-[6-(1-methyl-1H-pyrazol-4-yl)pyrazolo[1,5-a]pyridine-3-yl]-1,4-diazepane-1-carboxylate (Compound 40) as a yellow sticky foam. ¹H NMR (400 MHZ, CDCl₃): δ (ppm) 8.41 (s, 1H) 7.73 (s, 1H) 7.64 (s, 1H) 7.60 (s, 1H) 7.48 (d, J=9.29 Hz, 1H) 7.07 (d, J=9.05 Hz, 1H) 3.97 (s, 3H) 3.64-3.69 (m, 1H) 3.59-3.64 (m, 1H) 3.56 (t, J=6.11 Hz, 1H) 3.50 (t, J=6.17 Hz, 1H) 3.33 (br. s., 4H) 1.92-2.07 (m, 2H) 1.49 (s, 5H) 1.46 (s, 5H); MS (ESI) m/z [M+H]⁺ calcd. for C₂₁H₂₉N₆O₂: 397.2; found, 397.3; HPLC purity: 210 nm: 98.3%; 254 nm: 98.2%.

### Example 44: Synthesis of Exemplary Compound 41

### 3-[4-(4-benzylpyrimidin-2-yl)piperazin-1-yl]-6-(1-methyl-1H-pyrazol-4-yl)pyrazolo[1,5-a]pyridine (Compound 41)

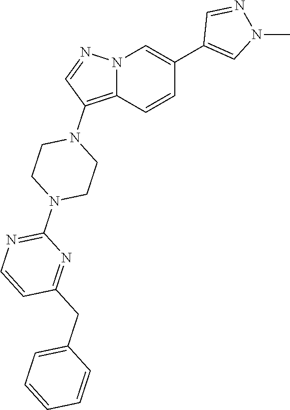 [SMILES: Cn1cc(-c2ccc3c(N4CCN(c5nccc(Cc6ccccc6)n5)CC4)cnn3c2)cn1]

A mixture of 6-(1-methyl-1H-pyrazol-4-yl)-3-piperazin-1-ylpyrazolo[1,5-a]pyridine dihydrochloride (Compound S19, 0.110 g, 0.310 mmol), 4-benzyl-2-chloropyrimidine (0.0760 g, 0.372 mmol) and potassium carbonate (0.171 g, 1.24 mmol) in N-methylpyrrolidinone (2.63 mL, 27.2 mmol) was heated in a 110° C. heating block under N₂ overnight. LCMS showed no starting material. The mixture was cooled to rt and diluted with 10 mL of water. The mixture was extracted with 3×10 mL of EtOAc. The combined EtOAc layers were washed with 2×10 mL of water and 10 mL of brine. Drying over Na₂SO₄ and evaporation of the solvent gave 0.15 g of a dark oil. The product was purified by reverse phase chromatography on a Combiflash® in 10% (Solvent B=0.07% TFA/CH₃CN)/(Solvent A=0.1% TFA/water) to 50% B over 35 CV hold for 30 CV on 0.5 g of C₁₈ silica gel. The fractions containing product were concentrated on the rotoevap to remove CH₃CN, then basified by addition of solid Na₂CO₃. The resulting cloudy suspension was extracted with 3×10 mL of EtOAc. The combined EtOAc layers were dried over Na₂SO₄ and evaporated to give 0.023 g (15%) of 3-[4-(4-benzylpyrimidin-2-yl)piperazin-1-yl]-6-(1-methyl-1H-pyrazol-4-yl)pyrazolo[1,5-a]pyridine (Compound 41) as a foam. ¹H NMR (400 MHZ, CDCl₃): δ (ppm) 8.49 (s, 1H), 8.22 (d, J=5.01 Hz, 1H), 7.76 (d, J=9.41 Hz, 2H), 7.51-7.66 (m, 2H), 7.24-7.37 (m, 5H), 7.17 (dd, J=9.17, 1.47 Hz, 1H), 6.35 (d, J=5.01 Hz, 1H), 4.03-4.11 (m, 4H), 3.93-4.03 (m, 4H), 3.11-3.19 (m, 4H), 1.28 (s, 3H); MS (ESI) m/z [M+H]⁺ calcd. for C₂₆H₂₇N₈: 451.5; found: 451.3; HPLC purity: 210 nm: 98.2%; 254 nm: 98.0%.

### Example 45: Synthesis of Exemplary Compound 42

### 6-(1-methyl-1H-pyrazol-4-yl)-3-{4-[5-(piperidin-1-ylmethyl)pyrimidin-2-yl]piperazin-1-yl}pyrazolo[1,5-a]pyridine (Compound 42)

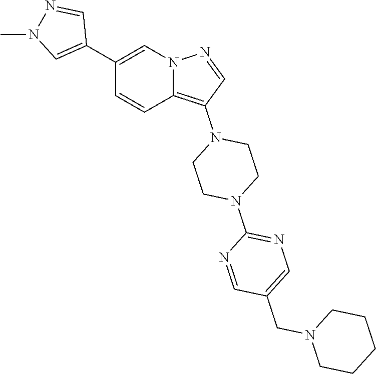 [SMILES: Cn1cc(-c2ccc3c(N4CCN(c5ncc(CN6CCCCC6)cn5)CC4)cnn3c2)cn1]

### Step 1. Preparation of tert-butyl 4-[5-(piperidin-1-ylmethyl)pyrimidin-2-yl]piperazine-1-carboxylate

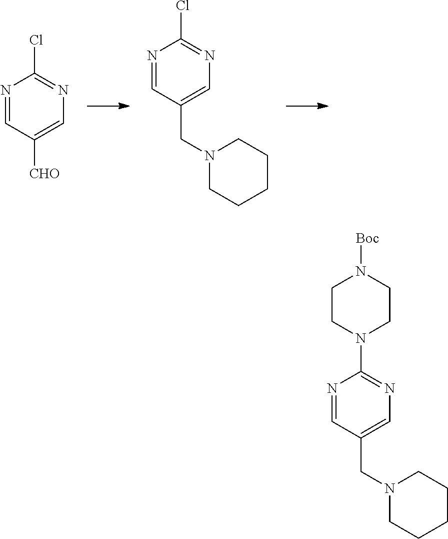 [SMILES: O=Cc1cnc(Cl)nc1 ; Clc1ncc(CN2CCCCC2)cn1 ; CC(C)(C)OC(=O)N1CCN(c2ncc(CN3CCCCC3)cn2)CC1]

To a cooled (0-5° C.) mixture of 2-chloropyrimidine-5-carbaldehyde (0.550 g, 3.86 mmol) in 1,2-dichloroethane (10 mL) and THF (3 mL) was added acetic acid (0.199 mL, 3.51 mmol) and piperidine (0.347 mL, 3.51 mmol) followed by sodium triacetoxyborohydride (1.04 g, 4.91 mmol). After 10 min the ice bath was removed, and the mixture stirred at ambient temperature overnight. The mixture was diluted with DCM and the organics washed with satd aq. NaHCO₃ and water, dried (Na₂SO₄), filtered and concentrated to afford 2-chloro-5-(piperidin-1-ylmethyl)pyrimidine that was used in the next step without purification. MS (LCMS) m/z [M+H]⁺ calcd. for C₁₀H₁₅ClN₃, 212.1; found, 212.1.

To a solution of 2-chloro-5-(piperidin-1-ylmethyl)pyrimidine (0.370 g, 1.75 mmol) in N-methylpyrrolidone (1 mL) was added tert-butyl 1-piperazinecarboxylate (0.359 g, 1.93 mmol) and potassium carbonate (0.363 g, 2.63 mmol). The mixture was heated to 80° C. for 1 h and then at ambient temperature overnight. The mixture was diluted with DCM and the organics washed with satd aq. NaHCO₃ and water, dried (Na₂SO₄), filtered and concentrated. The residue was concentrated several times from water and toluene and then purified by Biotage chromatography (40 g normal phase cartridge) eluting with a gradient of 0-100% EtOAc (containing 0.1% TEA) in hexanes to afford 0.240 g (38%, 2 steps) of tert-butyl 4-[5-(piperidin-1-ylmethyl)pyrimidin-2-yl]piperazine-1-carboxylate as a white solid. ¹H NMR (400 MHZ, CDCl₃): δ (ppm) 8.26 (s, 2H) 3.75-3.87 (m, 4H) 3.48-3.57 (m, 4H) 3.33 (s, 2H) 2.37 (br. s., 4H) 1.54-1.59 (m, 4H) 1.51 (s, 9H) 1.45 (m, 2H); MS (ESI) m/z [M+H]⁺ calcd. for C₁₉H₃₂N₅O₂, 362.3; found, 362.3; HPLC purity: 210 nm: 100.0%; 254 nm: 96.9%.

### Step 2. Preparation of 2-piperazin-1-yl-5-(piperidin-1-ylmethyl)pyrimidine

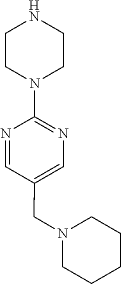 [SMILES: c1nc(N2CCNCC2)ncc1CN1CCCCC1]

To a vial containing tert-butyl 4-[5-(piperidin-1-ylmethyl)pyrimidin-2-yl]piperazine-1-carboxylate (0.405 g, 1.12 mmol) was added Dowex 50W4-200 (1.2 g, previously washed 2 times with DMF, 2 times with water, and alternatively with DCM and MeOH 3 times then dried in vacuo for 2h) and methanol (12 mL). The mixture was heated to 50° C. with gentle stirring and occasional swirling to ensure good mixing for 3 h. The mixture was cooled and filtered. The resin was washed with DCM, MeOH, DCM, MeOH. These filtrates were discarded. The compound was eluted from the resin by slow filtration (gravity) with 3.5N NH₃/MeOH (3×30 mL). The resin was washed 2 times with MeOH, 2 times with DCM and 2 times with MeOH. The combined filtrates were concentrated and the residue concentrated 2 times from MeOH and dried in vacuo to provide 280 mg (96%) of 2-piperazin-1-yl-5-(piperidin-1-ylmethyl)pyrimidine as a tan solid. ¹H NMR (400 MHZ, CDCl₃): δ (ppm) 8.22 (s, 2H) 3.63-3.92 (m, 4H) 3.29 (s, 2H) 2.81-3.05 (m, 4H) 2.33 (br. s., 4H) 1.47-1.66 (m, 4H) 1.25-1.47 (m, 2H); MS (ESI) m/z [M+H]⁺ calcd. for C₁₄H₂₄N₅, 262.2; found, 262.2.

### Step 3. Preparation of 6-(1-methyl-1H-pyrazol-4-yl)-3-{4-[5-(piperidin-1-ylmethyl)pyrimidin-2-yl]piperazin-1-yl}pyrazolo[1,5-a]pyridine (Compound 42)

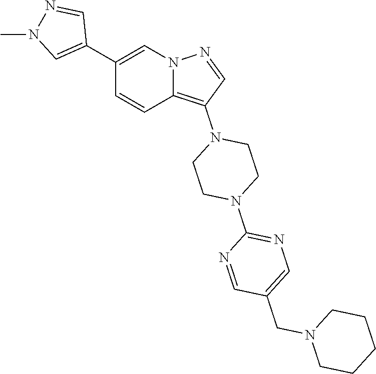 [SMILES: Cn1cc(-c2ccc3c(N4CCN(c5ncc(CN6CCCCC6)cn5)CC4)cnn3c2)cn1]

To a flame dried flask was added 3-bromo-6-(1-methyl-1H-pyrazol-4-yl)pyrazolo[1,5-a]pyridine (Compound S9, 0.326 g, 1.18 mmol) and 2-piperazin-1-yl-5-(piperidin-1-ylmethyl)pyrimidine (0.280 g, 1.07 mmol) followed by tert-butyl alcohol (9 mL) and 1,4-dioxane (4.4 mL). The solution was sparged with argon for 5 min and sodium tert-butoxide (0.154 g, 1.61 mmol) added. The mixture was sparged for 5 min with argon and tBuXPhos Pd G1 (74 mg, 0.11 mmol) added. The mixture was sparged for 5 min with argon and an additional 2 min with sonication and heated at 55° C. under an atmosphere of argon for 4 h. The reaction was cooled to ambient temperature and diluted with DCM. The organics were washed with satd aq NaHCO₃, brine, dried (Na₂SO₄) filtered and concentrated. The residue was purified by Biotage chromatography (40 g normal phase cartridge) eluting with a gradient of 0-10% of methanol (containing 0.1% triethylamine) in DCM to afford 0.090 g, (16%) of 6-(1-methyl-1H-pyrazol-4-yl)-3-{4-[5-(piperidin-1-ylmethyl)pyrimidin-2-yl]piperazin-1-yl}pyrazolo[1,5-a]pyridine (Compound 42) as a yellow solid. ¹H NMR (400 MHZ, CDCl₃): δ (ppm) 8.48 (s, 1H) 8.30 (s, 2H) 7.76 (s, 1H) 7.74 (s, 1H) 7.63 (s, 1H) 7.58 (d, J=9.17 Hz, 1H) 7.17 (dd, J=9.17, 1.47 Hz, 1H) 4.02-4.12 (m, 4H) 3.99 (s, 3H) 3.37 (s, 2H) 3.08-3.21 (m, 4H) 2.41 (br. s., 4H) 1.54-1.72 (m, 7H) 1.39-1.52 (m, 2H); MS (ESI) m/z [M+H]⁺ calcd. for C₂₅H₃₂N₉, 458.3; found, 458.3; HPLC purity: 210 nm: 100.0%; 254 nm: 100.0%.

### Example 46: Synthesis of Exemplary Compound 43

### 2-(4-{3-[4-(5-benzylpyrimidin-2-yl)piperazin-1-yl]pyrazolo[1,5-a]pyridin-6-yl}-1H-pyrazol-1-yl)-N,N-dimethylethanamine (Compound 43)

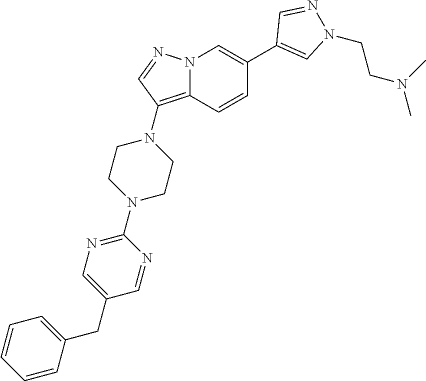 [SMILES: CN(C)CCn1cc(-c2ccc3c(N4CCN(c5ncc(Cc6ccccc6)cn5)CC4)cnn3c2)cn1]

### Step 1. Preparation of N, N-dimethyl-2-(4-pyrazolo[1,5-a]pyridin-6-yl-1H-pyrazol-1-yl)ethanamine

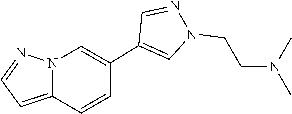 [SMILES: CN(C)CCn1cc(-c2ccc3ccnn3c2)cn1]

6-bromopyrazolo[1,5-a]pyridine (0.297 g, 1.51 mmol), N,N-dimethyl-2-[4-(4,4,5,5-tetramethyl-1,3,2-dioxaborolan-2-yl)-1H-pyrazol-1-yl]ethanamine (0.500 g, 1.88 mmol), potassium carbonate (0.417 g, 3.02 mmol), 1,4-dioxane (2.94 mL, 37.7 mmol), and water (0.595 mL, 33.0 mmol) were placed into a 20 mL vial with a magnetic stir bar and a rubber septum stopper and the mixture was sparged with argon for 20 minutes. Tetrakis(triphenylphosphine) palladium (0) (0.098 g, 0.084 mmol) was added and the mixture was sparged for another 5 minutes. The mixture was heated at 80° C. under argon for 3 h. LCMS showed no starting boronic acid. The mixture was cooled to rt and partitioned between water (20 mL) and CH₂Cl₂. The layers were separated and the CH₂Cl₂ layer was extracted with 3×20 mL of water. The combined organic layers were washed with 2×20 mL of water and dried over Na₂SO₄. Evaporation gave 0.5 g of a yellow oil. Chromatography using an Isolera® on 12 g of silica gel using a gradient of 0% solvent B (10% MeOH/CH₂Cl₂) in solvent A (CH₂Cl₂) to 100% solvent B over 15 column volumes gave 0.3 g (78%) of N, N-dimethyl-2-(4-pyrazolo[1,5-a]pyridin-6-yl-1H-pyrazol-1-yl)ethanamine as a clear oil. ¹H NMR (400 MHZ, CDCl₃): δ (ppm) 8.61-8.66 (m, 1H), 7.94 (d, J=2.32 Hz, 1H), 7.74-7.82 (m, 2H), 7.51-7.59 (m, 1H), 7.24-7.31 (m, 1H), 6.50-6.55 (m, 1H), 4.23-4.32 (m, 2H), 2.76-2.86 (m, 2H), 2.18-2.35 (m, 6H); MS (ESI) m/z [M+H]⁺ calcd. for C₁₄H₁₈N₅: 256.3; found: 256.2; HPLC purity: 210 nm: 100.0%; 254 nm: 100.0%.

### Step 2. Preparation of 2-[4-(3-bromopyrazolo[1,5-a]pyridin-6-yl)-1H-pyrazol-1-yl]-N,N-dimethylethanamine

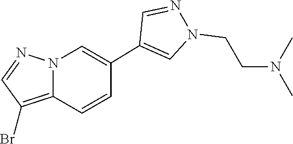 [SMILES: CN(C)CCn1cc(-c2ccc3c(Br)cnn3c2)cn1]

N-Bromosuccinimide (0.167 g, 0.94 mmol) was added to a solution of N, N-dimethyl-2-(4-pyrazolo[1,5-a]pyridin-6-yl-1H-pyrazol-1-yl)ethanamine (0.200 g, 0.78 mmol) in 5 mL of dry DMF at rt under N₂. The mixture was stirred at rt for 2 h. HPLC showed no starting material. The reaction was quenched by addition of 10 mL of sat′d Na₂S₂O₃ and 10 mL of sat′d NaHCO₃. The suspension was stirred at rt for 20 min, then extracted with 2×50 mL of EtOAc. The combined EtOAc layers were washed with 3×10 mL of water and 10 mL of brine. Drying over Na₂SO₄ and evaporation gave 0.21 g of a light brown oil. Chromatography on an Isolera® in 0% solvent B (10% MeOH/CH₂Cl₂)/CH₂Cl₂ to 100% B over 15 column volumes on 4 g of silica gel gave 0.14 g (54%) of 2-[4-(3-bromopyrazolo[1,5-a]pyridin-6-yl)-1H-pyrazol-1-yl]-N,N-dimethylethanamine as a white wax. ¹H NMR (400 MHZ, CDCl₃): δ (ppm) 8.57 (s, 1H), 7.92 (s, 1H), 7.78 (d, J=4.28 Hz, 1H), 7.53 (dd, J=9.17, 0.73 Hz, 1H), 7.36 (dd, J=9.17, 1.47 Hz, 1H), 7.28 (s, 1H), 4.29 (t, J=6.48 Hz, 1H), 2.83 (t, J=6.48 Hz, 1H), 2.29-2.50 (m, 5H), 1.62 (s, 3H); MS (ESI) m/z [M+H]⁺ calcd. for C₁₄H₁₇BrN₅: 335.2; found: 336.1; HPLC purity: 210 nm: 92.2%; 254 nm: 94.5%.

### Step 3. Preparation of 2-(4-{3-[4-(5-benzylpyrimidin-2-yl)piperazin-1-yl]pyrazolo[1,5-a]pyridin-6-yl}-1H-pyrazol-1-yl)-N,N-dimethylethanamine (Compound 43)

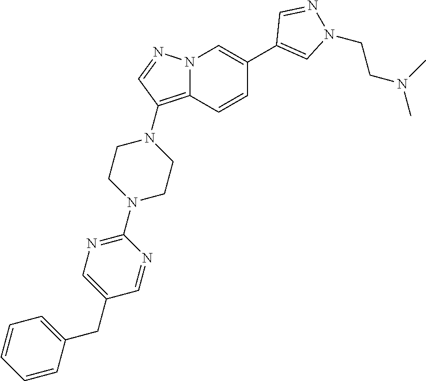 [SMILES: CN(C)CCn1cc(-c2ccc3c(N4CCN(c5ncc(Cc6ccccc6)cn5)CC4)cnn3c2)cn1]

A mixture of 2-[4-(3-bromopyrazolo[1,5-a]pyridin-6-yl)-1H-pyrazol-1-yl]-N,N-dimethylethanamine (0.070 g, 0.21 mmol), 5-benzyl-2-piperazin-1-ylpyrimidine (Compound S16, 0.064 g, 0.25 mmol), tert-butyl alcohol (1.54 mL, 16.1 mmol) and 1,4-dioxane (0.392 mL, 5.03 mmol) was sparged with Ar gas for 15 min at rt. tBuXPhos Pd G1 (0.0144 g, 0.0209 mmol) and sodium tert-butoxide (0.024 g, 0.25 mmol) were added and the mixture was sparged with Ar gas for 10 min. The mixture was heated in a 55° C. oil bath for 48 h. The catalyst was removed by filtration through Celite 545R and the filter cake was washed with 40 mL of CH₂Cl₂. The filtrate was washed with 2×40 mL of sat′d NaHCO₃ and 40 mL of brine. Drying over Na₂SO₄ and evaporation of the solvent gave 0.10 g of a red solid. Chromatography on an Isolera® in 0% acetone/CH₂Cl₂ to 50% acetone/CH₂Cl₂ over 12 column volumes on 4 g of silica gel gave 0.05 g of a waxy solid labeled 2106-TRB-60. HPLC showed 47% product along with 42% bromide at 2.6 min and 8.5% of an impurity at 2.3 min. Reverse phase chromatography on a CombiFlash® in 10% B (0.07% TFA/CH₃CN)/A (2.8% TFA/water) to 100% B over 40 CV on 4 g of C₁₈ silica gel gave 0.02 g of a hydroscopic solid. The solid was taken up in 5 mL of MeOH and the resulting solution was treated with MP-Carbonate resin (10 mg) at rt for 1 h. The resin was removed by filtration through Celite 545R. The filter cake was washed with 10 mL of MeOH. The filtrate was evaporated to give 7 mg (6%) of 2-(4-{3-[4-(5-benzylpyrimidin-2-yl)piperazin-1-yl]pyrazolo[1,5-a]pyridin-6-yl}-1H-pyrazol-1-yl)-N,N-dimethylethanamine (Compound 43) as a light yellow solid. ¹H NMR (400 MHZ, CDCl₃): δ (ppm) 8.49 (s, 1H), 8.23 (s, 2H), 7.72-7.79 (m, 3H), 7.57 (d, J=9.17 Hz, 1H), 7.16-7.34 (m, 6H), 4.29 (t, J=6.54 Hz, 2H), 3.98-4.08 (m, 4H), 3.83 (s, 2H), 3.11-3.25 (m, 4H), 2.82 (t, J=6.60 Hz, 2H), 2.32 (s, 6H); MS (ESI) m/z [M+H]⁺ calcd. for C₂₉H₃₄N₉: 508.6; found: 508.4; HPLC purity: 210 nm: 95.7%; 254 nm: 96.2%.

### Example 47: Synthesis of Exemplary Compound 44

### Tert-butyl 4-[6-(1-methyl-1H-pyrazol-4-yl)pyrazolo[1,5-a]pyrazin-3-yl)piperazine-1-carboxylate (Compound 44)

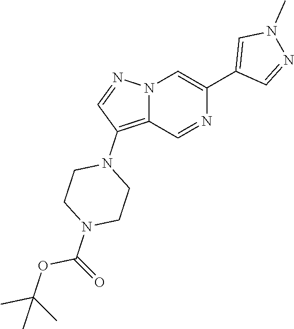 [SMILES: Cn1cc(-c2cn3ncc(N4CCN(C(=O)OC(C)(C)C)CC4)c3cn2)cn1]

A solution of 3-bromo-6-(1-methyl-1H-pyrazol-4-yl)pyrazolo[1,5-a]pyrazine (Compound S5, 200.0 mg, 0.72 mmol) and tert-butyl 1-piperazinecarboxylate (161 mg, 0.86 mmol) in tert-butanol (5.3 mL) and 1,4-dioxane (1.4 mL) was sparged for 20 min with argon. The solution was treated with tBuXPhos Pd G1 (74 mg, 0.11 mmol) and sodium tert-butoxide (104 mg, 1.08 mmol) and sparged for 10 min with argon. The resultant mixture was heated at 55° C. for 16.5 h at which time the reaction was determined to be complete. The mixture was cooled to rt, diluted with 40 mL EtOAc and washed with two 25 mL portions of H₂O. The organic phase was dried over MgSO₄ filtered and concentrated to yield a reddish brown residue that was purified by flash chromatography (60 g silica gel, 20-40% acetone/CH₂Cl₂) to yield 140 mg (51% yield) of tert-butyl 4-[6-(1-methyl-1H-pyrazol-4-yl)pyrazolo[1,5-a]pyrazin-3-yl)piperazine-1-carboxylate (Compound 44) as a yellow solid. ¹H NMR (400 MHZ, CDCl₃): δ (ppm) 9.00 (d, J=1.47 Hz, 1H) 8.37 (d, J=1.35 Hz, 1H) 7.88 (s, 1H) 7.88 (s, 1H) 7.66 (s, 1H) 3.98 (s, 3H) 3.57-3.76 (m, 4H) 3.02-3.19 (m, 4H) 1.51 (s, 9H); MS (ESI) m/z [M+H]⁺ calcd. for C₁₉H₂₆N₇O₂: 384.2; found, 384.3; HPLC purity: 210 nm: 100.0%; 254 nm: 100.0%.

### Example 48: Synthesis of Exemplary Compound 45

### Tert-butyl 4-[6-(1-methyl-1H-pyrazol-4-yl)pyrazolo[1,5-b]pyridazin-3-yl]piperazine-1-carboxylate (Compound 45)

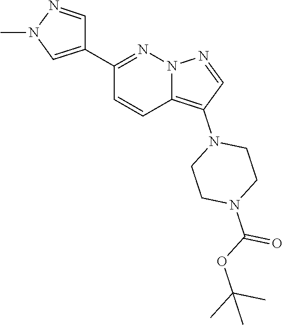 [SMILES: Cn1cc(-c2ccc3c(N4CCN(C(=O)OC(C)(C)C)CC4)cnn3n2)cn1]

A flask containing 3-bromo-6-(1-methyl-1H-pyrazol-4-yl)pyrazolo[1,5-b]pyridazine (Compound S8, 0.100 g, 0.36 mmol) and tert-butyl 1-piperazinecarboxylate (0.080 g, 0.43 mmol) was treated with tert-butyl alcohol (1.31 mL) and 1,4-dioxane (0.337 mL). The solution was sparged with argon gas for 15 min. The solution was treated with tBuXPhos Pd G1 (0.037 g, 0.054 mmol) and sodium tert-butoxide (0.042 g, 0.43 mmol) and the mixture was sparged again for 10 min with argon gas then heated in a 55° C. heating block. LCMS showed 7% conversion to tert-butyl 4-[6-(1-methyl-1H-pyrazol-4-yl)pyrazolo[1,5-b]pyridazin-3-yl]piperazine-1-carboxylate. The mixture was heated overnight. LCMS showed 24% conversion. The mixture was cooled to rt and the catalyst was removed by filtration through Celite 545R. The filter cake was rinsed with 20 mL of CH₂Cl₂. The filtrate was extracted with 2×20 mL of sat′d NaHCO₃ and 20 mL of brine. Drying over Na₂SO₄ and evaporation gave 0.12 g of a yellow oil. Chromatography on an Isolera® in 0% acetone/CH₂Cl₂ to 80% acetone/CH₂Cl₂ over 15 column volumes on 12 g of silica gel gave 0.032 g (23%) of tert-butyl 4-[6-(1-methyl-1H-pyrazol-4-yl)pyrazolo[1,5-b]pyridazin-3-yl]piperazine-1-carboxylate (Compound 45) as a yellow foam. ¹H NMR (400 MHZ, CDCl₃): δ (ppm) 8.03 (d, J=5.38 Hz, 2H), 7.91 (d, J=9.29 Hz, 1H), 7.70 (s, 1H), 7.08 (d, J=9.29 Hz, 1H), 4.00 (s, 3H), 3.58-3.72 (m, 4H), 3.05 (d, J=9.90 Hz, 4H), 1.52 (s, 9H), MS (ESI) m/z [M+H]⁺ calcd. for C₁₉H₂₆N₇O₂: 384.4; found: 384.3; HPLC purity: 210 nm: 100.0%; 254 nm: 100.0%.

### Example 49: Synthesis of Exemplary Compound 46

### 1-(4-fluorophenyl)-1-(2-{4-[6-(1-methyl-1H-pyrazol-4-yl)pyrazolo[1,5-a]pyridin-3-yl]piperazin-1-yl}pyrimidin-5-yl)ethanol (Compound 46)

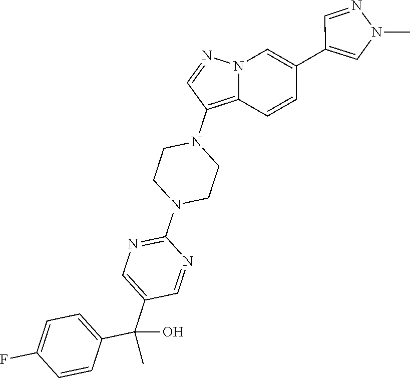 [SMILES: Cn1cc(-c2ccc3c(N4CCN(c5ncc(C(C)(O)c6ccc(F)cc6)cn5)CC4)cnn3c2)cn1]

To a cooled (−78° C.) mixture of (4-fluorophenyl)(2-{4-[6-(1-methyl-1H-pyrazol-4-yl)pyrazolo[1,5-a]pyridin-3-yl]piperazin-1-yl}pyrimidin-5-yl)methanone (Compound 25, 0.100 g, 0.21 mmol) in THF (5 mL) was added methylmagnesium chloride (3 M in THF, 0.138 mL, 0.41 mmol). The mixture was diluted with additional THF (5 mL) and allowed to warm to rt. The reaction was monitored by HPLC and additional methylmagnesium chloride added portion wise until the reaction was deemed complete. The mixture was cooled in an ice bath and quenched by the addition of 0.1 N HCl. The mixture was extracted 3 times with ethyl acetate and the combined organics washed with 0.1 N HCl, brine, dried (Na₂SO₄) filtered and concentrated. The residue was purified by Biotage chromatography (12 g normal phase cartridge) eluting with a gradient of 0-10% methanol in DCM to afford 0.070 g, (68%) of 1-(4-fluorophenyl)-1-(2-{4-[6-(1-methyl-1H-pyrazol-4-yl)pyrazolo[1,5-a]pyridin-3-yl]piperazin-1-yl}pyrimidin-5-yl)ethanol (Compound 46) as a grey solid. ¹H NMR (400 MHZ, CDCl₃): δ (ppm) 8.48 (s, 1H) 8.38 (s, 2H) 7.76 (s, 1H) 7.72 (s, 1H) 7.63 (s, 1H) 7.57 (d, J=9.29 Hz, 1H) 7.39-7.48 (m, 2H) 7.17 (dd, J=9.17, 1.34 Hz, 1H) 7.01-7.12 (m, 2H) 4.02-4.11 (m, 4H) 3.99 (s, 3H) 3.07-3.20 (m, 4H) 2.21 (br. s., 1H) 1.95 (s, 3H); MS (ESI) m/z [M+H]⁺ calcd. for C₂₇H₂₈FN₈O, 499.2; found, 499.3; HPLC purity: 210 nm: 95.0%; 254 nm: 96.4%.

### Example 50: Synthesis of Exemplary Compound 47

### N-isopropyl 4-[6-(1-methyl-1H-pyrazol-4-yl)pyrazolo[1,5-a]pyridin-3-yl]piperazine-1-carboxamide (Compound 47)

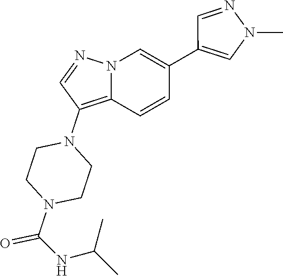 [SMILES: CC(C)NC(=O)N1CCN(c2cnn3cc(-c4cnn(C)c4)ccc23)CC1]

To a glass vial containing 6-(1-methyl-1H-pyrazol-4-yl)-3-piperazin-1-ylpyrazolo[1,5-a]pyridine dihydrochloride (Compound S19, 125 mg, 0.35 mmol) was added N,N-dimethylacetamide (3.8 mL) and diisopropylethylamine (299 μL, 1.72 mmol). 2-Isocyanatopropane (38 μL, 0.39 mmol) was added dropwise with rapid stirring. The reaction was monitored by HPLC, and dosed as needed with additional 2-isocyanatopropane until all 6-(1-methyl-1H-pyrazol-4-yl)-3-piperazin-1-ylpyrazolo[1,5-a]pyridine was consumed. The reaction mixture was diluted with DCM and washed with water (2 times) and brine. Solvent was mostly removed under vacuum and the residue was purified by Biotage chromatography (12 g normal phase cartridge) eluting with a gradient of 0-95% acetone (0.1% Et₃N additive) in DCM to afford 91 mg (70%) of 4-[6-(1-methyl-1H-pyrazol-4-yl)pyrazolo[1,5-a]pyridin-3-yl]piperazine-1-carboxamide (Compound 47) as a solid. ¹H NMR (400 MHZ, CDCl₃) δ 8.47 (s, 1H), 7.74 (s, 1H), 7.71 (s, 1H), 7.61 (s, 1H), 7.52 (d, J=8.07 Hz, 1H), 7.15 (dd, J=9.17 Hz, 1.22 Hz, 1H), 4.27 (d, J=6.97 Hz, 1H), 4.02 (m, 1H), 3.98 (s, 3H), 3.56 (m, 4H), 3.06 (m, 4H), 1.20 (d, J=6.48 Hz, 6H); MS (ESI) m/z [M+H]⁺ calcd. for C₁₉H₂₆N₇O: 368.2. Found: 368.3; HPLC purity: 210 nm: 100%; 254 nm: 100%.

### Example 51: Synthesis of Exemplary Compound 48

### 3-[6-(5-benzylpyrimidin-2-yl)-2,6-diazaspiro[3.3]hept-2-yl]-6-(1-methyl-pyrazol-4-yl)pyrazolo[1,5-a]pyridine (Compound 48)

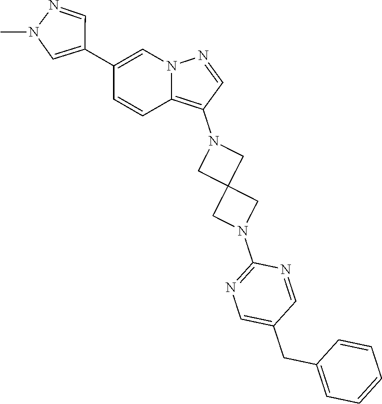 [SMILES: Cn1cc(-c2ccc3c(N4CC5(CN(c6ncc(Cc7ccccc7)cn6)C5)C4)cnn3c2)cn1]

### Step 1. Preparation of tert-butyl 6-[6-(1-methyl-1H-pyrazol-4-yl)pyrazolo[1,5-a]pyridin-3-yl]-2,6-diazaspiro[3.3]heptane-2-carboxylate

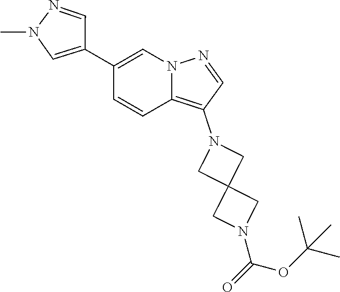 [SMILES: Cn1cc(-c2ccc3c(N4CC5(CN(C(=O)OC(C)(C)C)C5)C4)cnn3c2)cn1]

A flask containing 3-bromo-6-(1-methyl-1H-pyrazol-4-yl)pyrazolo[1,5-a]pyridine (Compound S9, 0.175 g, 0.63 mmol) and tert-butyl 2,6-diazaspiro[3.3]heptane-2-carboxylate (0.150 g, 0.76 mmol) was treated with tert-butyl alcohol (1.57 mL, 16.4 mmol) and 1,4-dioxane (0.394 mL, 5.04 mmol). The solution was sparged with argon gas for 15 min. The solution was treated with tBuXPhos Pd G1 (0.065 g, 0.095 mmol) and sodium tert-butoxide (0.073 g, 0.76 mmol) and the mixture was sparged again for 10 min with argon gas then heated in a 55° C. heating block overnight. The mixture was cooled to rt and the catalyst was removed by filtration through Celite 545R. The filter cake was rinsed with 20 mL of CH₂Cl₂. The filtrate was extracted with 2×20 mL of sat′d NaHCO₃ and 20 mL of brine. Drying over Na₂SO₄ and evaporation gave 0.52 g of a yellow oil. Chromatography on an Isolera® in 0% acetone/CH₂Cl₂ to 80% acetone/CH₂Cl₂ over 15 CV on 20 g of silica gel gave 0.2 g (80%) of tert-butyl 6-[6-(1-methyl-1H-pyrazol-4-yl)pyrazolo[1,5-a]pyridin-3-yl]-2,6-diazaspiro[3.3]heptane-2-carboxylate as a yellow foam. ¹H NMR (400 MHz, CDCl₃): δ (ppm) 8.40 (s, 1H), 7.74 (s, 1H), 7.60 (s, 1H), 7.46 (s, 1H), 7.37 (d, J=9.17 Hz, 1H), 6.96-7.13 (m, 1H), 4.14 (s, 4H), 4.05 (s, 4H), 3.98 (s, 3H), 1.48 (s, 9H); MS (ESI) m/z [M+H]⁺ calcd. for C₂₁H₂₇N₆O₂: 395.5; found: 395.3; HPLC purity: 210 nm: 77.7%; 254 nm: 92.0%.

### Step 2. Preparation of 3-(2,6-diazaspiro[3.3]hept-2-yl)-6-(1-methyl-1H-pyrazol-4-yl)pyrazolo[1,5-a]pyridine dihydrochloride

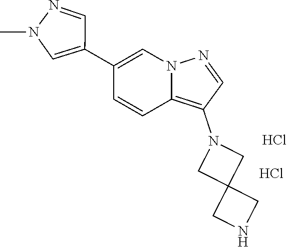 [SMILES: Cn1cc(-c2ccc3c(N4CC5(CNC5)C4)cnn3c2)cn1 ; Cl ; Cl]

A 4.0 M solution of hydrogen chloride in 1,4-dioxane (1.52 mL, 6.08 mmol) was added to a solution of tert-butyl 6-[6-(1-methyl-1H-pyrazol-4-yl)pyrazolo[1,5-a]pyridin-3-yl]-2,6-diazaspiro[3.3]heptane-2-carboxylate (0.200 g, 0.51 mmol) in methanol (3.88 mL, 95.8 mmol) at rt and the mixture was stirred at rt for 4 h. The solid was collected by filtration and rinsed with 5 mL of MeOH. Drying under hi-vac at rt over P₂O₅ overnight gave 0.14 g (75%) of 3-(2,6-diazaspiro[3.3]hept-2-yl)-6-(1-methyl-1H-pyrazol-4-yl)pyrazolo[1,5-a]pyridine dihydrochloride as a grey solid. ¹H NMR (300 MHz, DMSO-d₆): δ (ppm) 8.88 (s, 1H), 8.25 (s, 1H), 7.87-8.12 (m, 3H), 7.46 (d, J=9.03 Hz, 1H), 4.25 (s, 2H), 3.96-4.17 (m, 2H), 3.76-3.90 (m, 5H), 3.64 (br. s., 2H); HPLC purity: 210 nm: 91.3%; 254 nm: 90.8%.

### Step 3. Preparation of 3-[6-(5-benzylpyrimidin-2-yl)-2,6-diazaspiro[3.3]hept-2-yl]-6-(1-methyl-1H-pyrazol-4-yl)pyrazolo[1,5-a]pyridine (Compound 48)

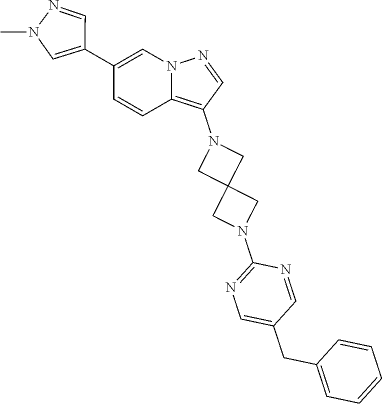 [SMILES: Cn1cc(-c2ccc3c(N4CC5(CN(c6ncc(Cc7ccccc7)cn6)C5)C4)cnn3c2)cn1]

A mixture of 3-(2,6-diazaspiro[3.3]hept-2-yl)-6-(1-methyl-1H-pyrazol-4-yl)pyrazolo[1,5-a]pyridine dihydrochloride (0.140 g, 0.38 mmol), 5-benzyl-2-chloropyrimidine (0.094 g, 0.46 mmol) and potassium carbonate (0.211 g, 1.52 mmol) in N-methylpyrrolidinone (3.23 mL, 33.5 mmol) was heated in a 110° C. heating block under N₂ for 2 h. LCMS showed no starting material. The mixture was cooled to rt and diluted with 10 mL of water. The mixture was extracted with 3×10 mL of EtOAc. The combined EtOAc layers were washed with 2×10 mL of water and 10 mL of brine. Drying over Na₂SO₄ and evaporation of the solvent gave 0.15 g of a dark oil. Chromatography on an Isolera® in 5% MeOH/CH₂Cl₂ on 20 g of silica gel gave 30 mg of material containing 2 products by TLC. The material was loaded onto a 1 ml silica gel prep plate (20 cm×20 cm) and eluted 3 times with 5% MeOH/CH₂Cl₂. The top spot was isolated to give 9 mg (5%) of 3-[6-(5-benzylpyrimidin-2-yl)-2,6-diazaspiro[3.3]hept-2-yl]-6-(1-methyl-1H-pyrazol-4-yl)pyrazolo[1,5-a]pyridine (Compound 48). ¹H NMR (400 MHZ, CDCl₃): δ (ppm) 8.41 (s, 1H), 8.22 (s, 2H), 7.74 (s, 1H), 7.60 (s, 1H), 7.48 (s, 1H), 7.40 (d, J=9.17 Hz, 1H), 7.16-7.34 (m, 5H), 7.03 (dd, J=9.17, 1.47 Hz, 1H), 4.33 (s, 4H), 4.14 (s, 4H), 3.98 (s, 3H), 3.83 (s, 2H); MS (ESI) m/z [M+H]⁺ calcd. for C₂₇H₂₇N₈: 463.5; found: 463.3; HPLC purity: 210 nm: 95.5%; 254 nm: 95.5%.

### Example 52: Synthesis of Exemplary Compound 49

### (R)-1-phenylethyl 4-(6-(1-methyl-1H-pyrazol-4-yl)pyrazolo[1,5-a]pyrazin-3-yl)piperazine-1-carboxylate (Compound 49)

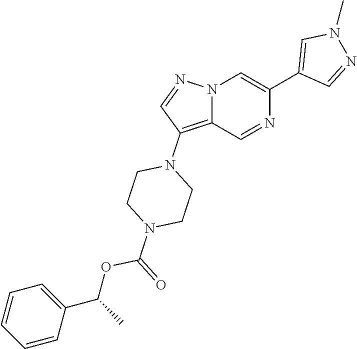 [SMILES: C[C@@H](OC(=O)N1CCN(c2cnn3cc(-c4cnn(C)c4)ncc23)CC1)c1ccccc1]

### Step 1: Preparation of 4-[6-(1-methyl-1H-pyrazol-4-yl)pyrazolo[1,5-a]pyrazin-3-yl]piperazin-1-ium trifluoroacetate

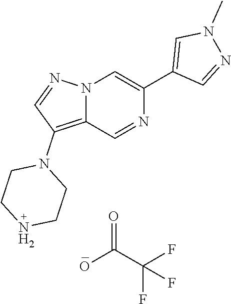

To an ice cooled solution of tert-butyl 4-[6-1-methyl-1H-pyrazol-4-yl)pyrazolo[1,5-a]pyrazin-3-yl)piperazine-1-carboxylate (Compound 44, 105 mg, 0.274 mmol) in CH₂Cl₂ (5.0 mL) was added TFA (0.40 mL, 5.2 mmol). The solution was stirred at 0° C. for 30 min followed by warming to rt. After 4 h the reaction was determined to be complete. The reaction mixture was concentrated, the residue taken up in 10 mL CH₂Cl₂ and concentrated. The process was repeated once and the resultant red residue was placed under high vac to yield crude 4-[6-(1-methyl-1H-pyrazol-4-yl)pyrazolo[1,5-a]pyrazin-3-yl]piperazin-1-ium trifluoroacetate that was found to be of suitable purity to be used in the next step. ¹H NMR (400 MHZ, CD₃OD): δ (ppm) 9.15 (s, 1H) 8.73 (d, J=1.35 Hz, 1H) 8.13 (s, 1H) 8.00 (s, 1H) 7.89 (s, 1H) 3.96 (s, 3H) 3.45-3.52 (m, 4H) 3.39-3.45 (m, 4H); MS (ESI) m/z [M+H]⁺ calcd. for C₁₄H₁₈N₇: 284.2; found, 284.1; HPLC purity: 210 nm: 97.3%; 254 nm: 99.1%.

### Step 2: Preparation of (R)-1-phenylethyl 4-(6-(1-methyl-1H-pyrazol-4-yl)pyrazolo[1,5-a]pyrazin-3-yl)piperazine-1-carboxylate (Compound 49)

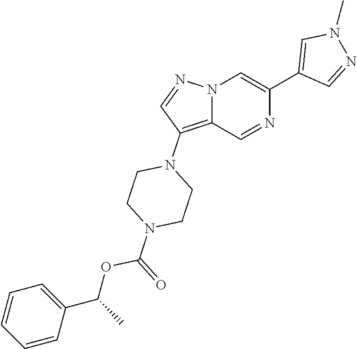 [SMILES: C[C@@H](OC(=O)N1CCN(c2cnn3cc(-c4cnn(C)c4)ncc23)CC1)c1ccccc1]

A solution of crude 4-[6-(1-methyl-1H-pyrazol-4-yl)pyrazolo[1,5-a]pyrazin-3-yl]piperazin-1-ium trifluoroacetate (109 mg, 0.27 mmol) in acetonitrile (3.0 mL) was treated dropwise with a solution of (R)-4-nitrophenyl (1-phenylethyl)carbonate (99 mg, 0.34 mmol) in acetonitrile (0.5 mL). The resultant solution was treated with N, N-diisopropylethylamine (0.33 mL, 1.92 mmol) and the reaction was stirred at rt for 1 h at which time the reaction was determined to be complete. The reaction was concentrated to a yellow oil that was purified by flash chromatography (60 g silica gel, 10-35% acetone/CH₂Cl₂) to yield 105 mg (88%) of (1R)-1-phenylethyl 4-[6-(1-methyl-1H-pyrazol-4-yl)pyrazolo[1,5-a]pyrazin-3-yl]piperazine-1-carboxylate (Compound 49) as a light yellow solid. ¹H NMR (400 MHZ, CDCl₃) δ ppm 9.01 (d, J=1.35 Hz, 1H) 8.39 (d, J=1.47 Hz, 1H) 7.90 (s, 1H) 7.89 (s, 1H) 7.67 (s, 1H) 7.39 (d, J=4.40 Hz, 4H) 7.29-7.37 (m, 1H) 5.89 (q, J=6.60 Hz, 1H) 3.99 (s, 3H) 3.76 (br. s., 4H) 3.01-3.26 (m, 4H) 1.60 (s, 3H); MS (ESI) m/z [M+H]⁺ calcd. for C₂₃H₂₆N₇O₂: 432.2; found, 432.2; HPLC purity: 210 nm: 100.0%; 254 nm: 100.0%.

### Example 53: Synthesis of Exemplary Compound 50

### (R)-1-phenylethyl 4-(6-(1-methyl-1H-pyrazol-4-yl)pyrazolo[1,5-b]pyridazin-3-yl)piperazine-1-carboxylate (Compound 50)

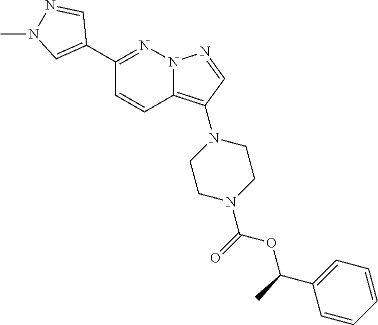 [SMILES: C[C@@H](OC(=O)N1CCN(c2cnn3nc(-c4cnn(C)c4)ccc23)CC1)c1ccccc1]

### Step 1. Preparation of 6-(1-methyl-1H-pyrazol-4-yl)-3-piperazin-1-ylpyrazolo[1,5-b]pyridazine dihydrochloride

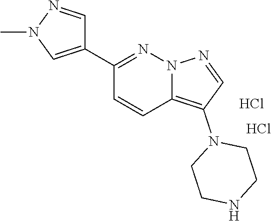 [SMILES: Cn1cc(-c2ccc3c(N4CCNCC4)cnn3n2)cn1 ; Cl ; Cl]

A 4.0 M solution of hydrogen chloride in 1,4-dioxane (1.64 mL, 6.57 mmol) was added to a solution of tert-butyl 4-[6-(1-methyl-1H-pyrazol-4-yl)pyrazolo[1,5-b]pyridazin-3-yl]piperazine-1-carboxylate (Compound 45, 0.210 g, 0.55 mmol) in methanol (4.19 mL, 10 mmol) at rt and the mixture was stirred at rt for 4 h. HPLC showed incomplete reaction. The mixture was stirred at rt overnight. The solid was collected by filtration and rinsed with 5 mL of MeOH. Drying under hi-vac at rt over P₂O₅ overnight gave 0.16 g (82%) of 6-(1-methyl-1H-pyrazol-4-yl)-3-piperazin-1-ylpyrazolo[1,5-b]pyridazine dihydrochloride as a grey solid. ¹H NMR (400 MHZ, DMSO-d₆): δ (ppm) 9.29 (br. s., 2H), 8.47 (s, 1H), 8.31 (d, J=9.29 Hz, 1H), 8.10 (s, 1H), 7.72-7.92 (m, 3H), 7.41 (d, J=9.29 Hz, 1H), 3.92 (s, 3H), 3.27 (s, 7H); MS (ESI) m/z [M+H]⁺ calcd. for C₁₄H₁₈N₇: 284.3; found: 284.2; HPLC purity: 210 nm: 100.0%; 254 nm: 100.0%.

### Step 3. Preparation of (R)-1-phenylethyl 4-(6-(1-methyl-1H-pyrazol-4-yl)pyrazolo[1,5-b]pyridazin-3-yl)piperazine-1-carboxylate (Compound 50)

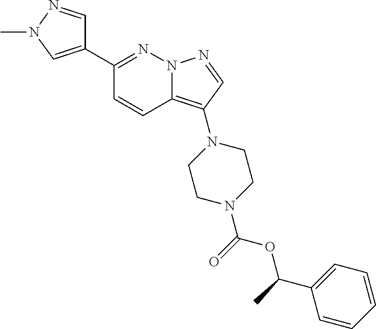 [SMILES: C[C@@H](OC(=O)N1CCN(c2cnn3nc(-c4cnn(C)c4)ccc23)CC1)c1ccccc1]

A suspension of 6-(1-methyl-1H-pyrazol-4-yl)-3-piperazin-1-ylpyrazolo[1,5-b]pyridazine dihydrochloride (123 mg, 0.35 mmol) in acetonitrile (3.8 mL, 72 mmol) was treated dropwise with a solution of (R)-4-nitrophenyl (1-phenylethyl)carbonate (124 mg, 0.43 mmol) in acetonitrile (0.6 mL, 10 mmol). The resultant suspension was treated dropwise with N, N-diisopropylethylamine (0.421 mL, 2.42 mmol) and the reaction mixture was allowed to stir at rt until complete as determined by HPLC. Upon addition of the DIEA the reaction mixture went from bright red to yellow. HPLC after ˜1 h indicated the starting amine had been consumed and a new product had formed. LCMS indicated likewise the SM had been consumed and clean conversion to the product. The reaction was concentrated to yellow oil and placed under high vac. Chromatography on an Isolera® in 0% acetone/CH₂Cl₂ to 80% acetone/CH₂Cl₂ over 15 CV on 4 g of silica gel gave 0.12 g (80%) (R)-1-phenylethyl 4-(6-(1-methyl-1H-pyrazol-4-yl)pyrazolo[1,5-b]pyridazin-3-yl)piperazine-1-carboxylate (Compound 50) as a yellow foam. ¹H NMR (400 MHZ, CDCl₃): δ (ppm) 8.03 (s, 1H), 8.02 (s, 1H), 7.90 (d, J=9.2 Hz, 1H), 7.70 (s, 1H), 7.28-7.41 (m, 5H), 7.08 (d, J=9.2 Hz, 1H), 5.88 (q, J=6.60 Hz, 1H), 4.00 (s, 3H), 3.73 (br. s., 4H), 3.05-3.08 (m, 4H), 1.58-1.67 (d, J=6.4 Hz, 3H); MS (ESI) m/z [M+H]⁺ calcd. for C₂₃H₂₆N₇O₂: 432.5; found: 432.3; HPLC purity: 210 nm; 98.5%; 254 nm: 100.0%.

### Example 54: Synthesis of Exemplary Compound 51

### 3-[1-(5-benzylpyrimidin-2-yl)-2,5-dihydro-1H-pyrrol-3-yl]-6-(1-methyl-1H-pyrazol-4-yl)pyrazolo[1,5-a]pyridine (Compound 51)

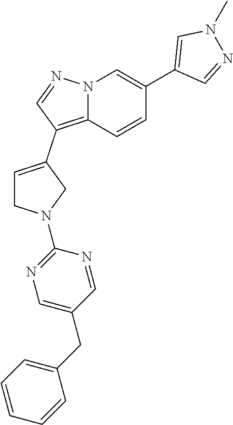 [SMILES: Cn1cc(-c2ccc3c(C4=CCN(c5ncc(Cc6ccccc6)cn5)C4)cnn3c2)cn1]

### Step 1. Preparation of tert-butyl 3-[6-(1-methyl-1H-pyrazol-4-yl)pyrazolo[1,5-a]pyridin-3-yl]-2,5-dihydro-1H-pyrrole-1-carboxylate

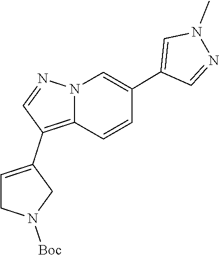 [SMILES: Cn1cc(-c2ccc3c(C4=CCN(C(=O)OC(C)(C)C)C4)cnn3c2)cn1]

A mixture of tert-butyl-3-(4,4,5,5-tetramethyl-1,3,2-dioxaborolan-2-yl)-2,5-dihydro-1H-pyrrole-carboxylate (100 mg, 0.34 mmol), 3-bromo-6-(1-methyl-1H-pyrazol-4-yl)pyrazolo[1,5-a]pyridine (Compound S9, 78 mg, 0.28 mmol) and potassium phosphate (120 mg, 0.57 mmol) in 1,4-dioxane (2.9 mL) and H₂O (1.0 mL) was sparged with argon for 10 min. The mixture was treated with bis(tri-tert-butylphosphine) palladium (0) (21 mg, 0.042 mmol) and the reaction mixture was heated at 80° C. for 16.5 h at which time the reaction was determined to be complete. The reaction was cooled to rt, diluted with 10 mL H₂O and extracted with two 15 mL portions of EtOAc. The combined organic phase was washed with 15 mL H₂O, dried over MgSO₄, filtered and concentrated to yield a brown oil that was purified by flash chromatography (30 g silica gel, 15-30% acetone/CH₂Cl₂) to yield 87 mg (84%) of tert-butyl 3-[6-(1-methyl-1H-pyrazol-4-yl)pyrazolo[1,5-a]pyridin-3-yl]-2,5-dihydro-1H-pyrrole-1-carboxylate as a grey solid. ¹H NMR (400 MHZ, CDCl₃): δ (ppm) 8.58 (d, J=3.42 Hz, 1H) 7.92 (d, J=12.72 Hz, 1H) 7.77 (s, 1H) 7.68 (dd, J=9.17, 3.06 Hz, 1H) 7.34 (dd, J=12.10, 9.29 Hz, 1H) 5.87-6.03 (m, 1H) 4.62 (br. s., 1H) 4.54 (br. s., 1H) 4.38 (br. s., 1H) 4.31 (br. s., 1H) 3.99 (s, 3H) 1.55 and 1.53 (rotomers s, 9H); MS (ESI) m/z [M+H]⁺ calcd. for C₂₀H₂₄N₅O₂: 366.2; found, 366.3; HPLC purity: 210 nm: 97.8%; 254 nm: 99.0%.

### Step 2. Preparation of 3-[6-(1-methyl-1H-pyrazol-4-yl)pyrazolo[1,5-a]pyridin-3-yl]-2,5-dihydro-1H-pyrrolium trifluoroacetate

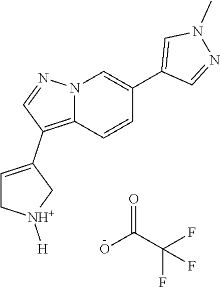 [SMILES: Cn1cc(-c2ccc3c(C4=CC[NH2+]C4)cnn3c2)cn1 ; O=C(O)C(F)(F)F]

A solution of tert-butyl 3-[6-(1-methyl-1H-pyrazol-4-yl)pyrazolo[1,5-a]pyridin-3-yl]-2,5-dihydro-1H-pyrrole-1-carboxylate (87 mg, 0.24 mmol) in CH₂Cl₂ (5.0 mL) was cooled at 0° C., and treated dropwise with TFA (0.40 mL, 5.2 mmol). The solution was stirred for 30 min at 0° C. following by warming to RT. After 1.5 h the starting material had been consumed. The reaction mixture was concentrated to a brown residue that was taken up in 7 mL CH₂Cl₂ and concentrated. The process was repeated once and the resultant residue placed under high vac to yield crude 3-[6-(1-methyl-1H-pyrazol-4-yl)pyrazolo[1,5-a]pyridin-3-yl]-2,5-dihydro-1H-pyrrolium trifluoroacetate that was determined to be of suitable purity for use in the next step (assumed a 100% yield). ¹H NMR (400 MHZ, CD₃OD): δ (ppm) 8.82 (s, 1H) 8.08 (s, 1H) 8.07 (s, 1H) 7.92 (s, 2H) 7.89 (s, 1H) 7.64 (dd, J=9.23, 1.53 Hz, 1H) 6.17 (t, J=2.02 Hz, 1H) 4.52 (d, J=2.08 Hz, 2H) 4.30 (d, J=2.08 Hz, 2H) 3.96 (s, 3H); MS (ESI) m/z [M+H]⁺ calcd. for C₁₅H₁₆N₅: 266.1; found, 266.2; HPLC purity: 210 nm: 89.3%; 254 nm: 85.6%.

### Step 3. Preparation of 3-[1-(5-benzylpyrimidine-2-yl)-2,5-dihydro-1H-pyrrol-3-yl]-6-(1-methyl-1H-pyrazol-4-yl)pyrazolo[1,5-a]pyridine (Compound 51)

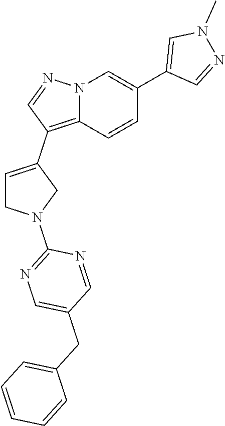 [SMILES: Cn1cc(-c2ccc3c(C4=CCN(c5ncc(Cc6ccccc6)cn5)C4)cnn3c2)cn1]

To a solution of crude 3-[6-(1-methyl-1H-pyrazol-4-yl)pyrazolo[1,5-a]pyridin-3-yl]-2,5-dihydro-1H-pyrrolium trifluoroacetate (90 mg, 0.23 mmol) in N-methylpyrrolidinone (2.0 mL) was added 5-benzyl-2-chloropyrimidine (51 mg, 0.25 mmol) and potassium carbonate (690 mg, 5.0 mmol) and the mixture was heated at 110° C. After 4 h it was determined that the starting material was consumed, the reaction mixture was cooled to RT, diluted with 25 mL EtOAc and washed with 15 mL H₂O. The organic phase was dried over MgSO₄, filtered and concentrated to a brownish yellow solid that was purified by flash chromatography (30 g silica gel, 2-4% MeOH/CH₂Cl₂) to yield 45 mg (44%) of 3-[1-(5-benzylpyrimidine-2-yl)-2,5-dihydro-1H-pyrrol-3-yl]-6-(1-methyl-1H-pyrazol-4-yl)pyrazolo[1,5-a]pyridine (Compound 51) as a light yellow solid. ¹H NMR (400 MHz, CDCl₃): δ (ppm) 8.59 (s, 1H) 8.27 (s, 2H) 8.01 (s, 1H) 7.73-7.82 (m, 2H) 7.65 (s, 1H) 7.29-7.38 (m, 3H) 7.15-7.25 (m, 3H) 6.12 (t, J=1.90 Hz, 1H) 4.74-4.88 (m, 2H) 4.50-4.65 (m, 2H) 3.99 (s, 3H) 3.84 (s, 2H); MS (ESI) m/z [M+H]⁺ calcd. for C₂₆H₂₄N₇: 434.2; found, 434.3; HPLC purity: 210 nm: 98.8%; 254 nm: 99.0%.

### Example 55: Synthesis of Exemplary Compound 52

### 3-[4-(5-benzylpyrimidin-2-yl)piperazin-1-yl]-7-(1-methyl-1H-pyrazol-4-yl)imidazo[1,2-b]pyridazine (Compound 52)

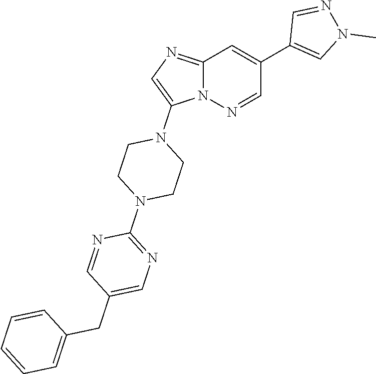 [SMILES: Cn1cc(-c2cnn3c(N4CCN(c5ncc(Cc6ccccc6)cn5)CC4)cnc3c2)cn1]

To a solution of 3-bromo-7-(1-methyl-1H-pyrazol-4-yl)imidazo[1,2-b]pyridazine (Compound S3, 75 mg, 0.27 mmol) in t-butanol (2.0 mL) and 1,4-dioxane (0.5 mL) was added 5-benzyl-2-piperazin-1-ylpyrimidine (Compound S16, 82 mg, 0.32 mmol) and the mixture was sparged with argon for 20 min. The mixture was further treated with tBuXPhos Pd G1 (28 mg, 0.040 mmol) and sodium t-butoxide (39 mg, 0.40 mmol) and the mixture was sparged for an additional 10 min with argon. The reaction mixture was heated at 55° C. for 2 h at which point it was determined by HPLC that the starting material had been consumed. The reaction mixture was cooled, diluted with 50 mL EtOAc and the solution was washed with two 25 mL portions of H₂O, followed by 10 mL brine. The organic phase was dried over Na₂SO₄, filtered, and concentrated to a yellowish brown solid. The crude product was purified by flash chromatography (30 g silica gel; 40-60% acetone/CH₂Cl₂ followed by 100% acetone) to yield 50 mg of a yellow solid that was not completely pure. The material was taken up in 0.75 mL hot DMF and the solution was cooled resulting in a solid that was filtered to afford 25 mg (20%) of 3-[4-(5-benzylpyrimidin-2-yl)piperazin-1-yl]-7-(1-methyl-1H-pyrazol-4-yl)imidazo[1,2-b]pyridazine (Compound 52) as a brown solid. ¹H NMR (400 MHZ, CDCl₃): δ (ppm) 8.50 (d, J=1.96 Hz, 1H) 8.22 (s, 2H) 7.79-7.89 (m, 2H) 7.72 (s, 1H) 7.28-7.36 (m, 3H) 7.11-7.25 (m, 3H) 4.04-4.13 (m, 4H) 4.00 (s, 3H) 3.82 (s, 2H) 3.24-3.43 (m, 4H); MS (ESI) m/z [M+H]⁺ calcd. for C₂₅H₂₆N₉, 452.2; found, 452.3; HPLC purity: 210 nm: 95.4%; 254 nm 99.0%.

### Example 56: Synthesis of Exemplary Compound 53

### 3-{4-[5-(3-methylbenzyl)pyrimidin-2-yl]piperazin-1-yl}-6-(1-methyl-1H-pyrazol-4-yl)pyrazolo[1,5-a]pyridine (Compound 53)

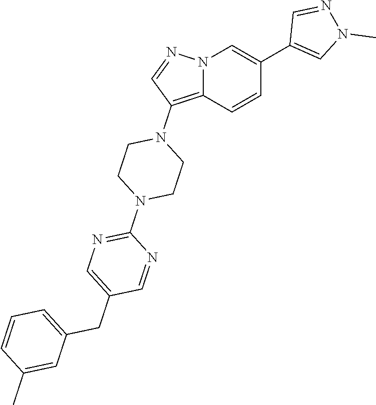 [SMILES: Cc1cccc(Cc2cnc(N3CCN(c4cnn5cc(-c6cnn(C)c6)ccc45)CC3)nc2)c1]

A vial containing 6-(1-methyl-1H-pyrazol-4-yl)-3-piperazin-1-ylpyrazolo[1,5-a]pyridine hydrochloride (Compound S19, 0.19 g, 80% w/w, 0.47 mmol) and 2-chloro-5-(3-methylbenzyl)pyrimidine (Compound S20, 0.15 g, 0.71 mmol) and DMF (4 mL) was warmed to 70° C. K₂CO₃ (0.32 g, 2.3 mmol) was added to the reaction and warmed to 100° C. while under an argon atmosphere. After stirring overnight at 100° C., the reaction was cooled to room temperature and filtered through a plug of Celite. The filter pad was washed with DCM and the filtrate concentrated. The crude was dissolved in DCM (60 mL) and washed with aq. LiCl (5×20 mL). The DCM layer was dried (Na₂SO₄) filtered and concentrated. The residue was purified by ISCO chromatography (40 g normal phase cartridge) eluting with a gradient of 0-100% EtOAc in hexanes followed by 10% MeOH in EtOAc to afford 0.14 g, (66%) of 3-{4-[5-(3-methylbenzyl)pyrimidin-2-yl]piperazin-1-yl}-6-(1-methyl-1H-pyrazol-4-yl)pyrazolo[1,5-a]pyridine (Compound 53) as a tan solid: ¹H NMR (400 MHZ, CDCl₃) δ (ppm) 8.47 (s, 1H) 8.22 (s, 2H) 7.75 (s, 1H) 7.72 (s, 1H) 7.62 (s, 1H) 7.56 (d, J=8.80 Hz, 1H) 7.12-7.23 (m, 2H) 7.04 (d, J=7.34 Hz, 1H) 6.96-7.01 (m, 2H) 3.95-4.06 (m, 7H) 3.78 (s, 2H) 3.09-3.17 (m, 4H) 2.33 (s, 3H); MS (ESI) m/z [M+H]⁺ calcd. for C₂₇H₂₉N₈, 465.3; found, 465.2; HPLC purity: 210 nm: 97.8%; 254 nm: 98.6%.

### Example 57: Synthesis of Exemplary Compound 54

### 3-{4-[5-(3-methoxybenzyl)pyrimidin-2-yl]piperazin-1-yl}-6-(1-methyl-1H-pyrazol-4-yl)pyrazolo[1,5-a]pyridine (Compound 54)

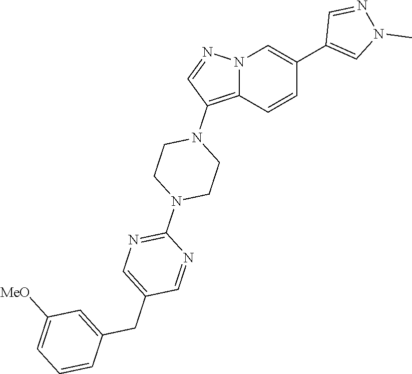 [SMILES: COc1cccc(Cc2cnc(N3CCN(c4cnn5cc(-c6cnn(C)c6)ccc45)CC3)nc2)c1]

A capped vial containing a well-stirred suspension of 6-(1-methyl-1H-pyrazol-4-yl)-3-piperazin-1-ylpyrazolo[1,5-a]pyridine hydrochloride (Compound S19, 0.18 g, 80% w/w, 0.44 mmol), 2-chloro-5-(3-methoxybenzyl)pyrimidine (Compound S21, 0.16 g, 0.66 mmol), DMF (4 mL), and K₂CO₃ (0.31 g, 2.2 mmol) was heated at 100° C. under a nitrogen atmosphere. After 16 h it was cooled to room temperature and partitioned between DCM and water (MeOH and NaHCO₃additives were helpful). Solvent was concentrated in the organic and it was purified by silica gel chromatography (12 g normal phase cartridge) eluted with a gradient of acetone in DCM. The resulting 111 mg solid was further purified by silica gel chromatography (12 g normal phase cartridge) eluted with a gradient of 2-propanol in DCM to afford 85 mg (40%) of 3-{4-[5-(3-methoxybenzyl)pyrimidin-2-yl]piperazin-1-yl}-6-(1-methyl-1H-pyrazol-4-yl)pyrazolo[1,5-a]pyridine (Compound 54) as a solid: ¹H NMR (400 MHZ, DMSO-d₆) δ (ppm) 8.47 (s, 1H) 8.22 (s, 2H) 7.75 (s, 1H) 7.73 (s, 1H) 7.62 (s, 1H) 7.57 (d, J=8.7 Hz, 1H) 7.23 (t, J=7.8 Hz), 1H) 7.15 (d, J=8.9 Hz, 1H) 6.77 (m, 2H), 6.72 (m, 1H) 4.02 (br. s., 4H) 3.98 (s, 3H), 3.79 (s, 5H) 3.13 (br. s., 4H); MS (ESI) m/z [M+H]⁺ calcd. for C₂₇H₂₉N₈O, 481.2; found, 481.2; HPLC purity: 210 nm: 97.5%; 254 nm: 98.8%.

### Example 58: Synthesis of Exemplary Compound 55

### 2-(4-{3-[4-(5-benzylpyrimidin-2-yl)piperazin-1-yl]pyrazolo[1,5-a]pyridin-6-yl}-1H-pyrazol-1-yl)ethanol (Compound 55)

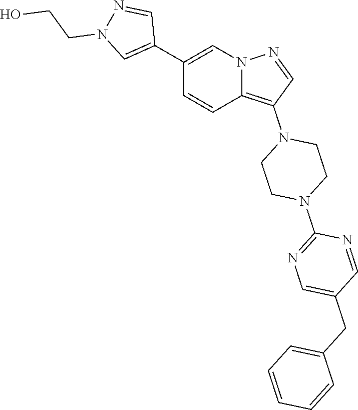 [SMILES: OCCn1cc(-c2ccc3c(N4CCN(c5ncc(Cc6ccccc6)cn5)CC4)cnn3c2)cn1]

2-[4-(3-piperazine-1-ylpyrazolo[1,5-a]pyridin-6-yl)-1H-pyrazol-1-yl]ethanol (Compound S22, 0.20 g, 0.63 mmol), 2-chloro-5-(4-fluorobenzyl)pyrimidine (Compound S28, 0.08 g, 0.25 mmol), K₂CO₃ (0.18 g, 1.27 mmol), and DMF (3 mL) were combined. The mixture was heated at 100° C. while under a nitrogen atmosphere for 7 hr. The reaction was cooled to room temperature and partitioned between DCM and water. Saturated aqueous ammonium chloride was added to provide a clean split, and it moved the pH from 12 to 8. Volatiles were removed at reduced pressure and the residue was loaded onto a 12 g silica gel cartridge and eluted with a gradient of 2-propanol/DCM to give 49 mg partially purified solid. Selected fractions from chromatography on another 12 g silica gel cartridge with a gradient of acetone (0.1% Et₃N)/DCM gave 17 mg (14%) of 3-{4-[5-(4-fluorobenzyl)pyrimidin-2-yl]piperazin-1-yl}-6-(1-methyl-1H-pyrazol-4-yl)pyrazolo[1,5-a]pyridine (Compound 55) as a pale yellow solid: ¹H NMR (400 MHZ, CDCl₃) δ (ppm) 8.48 (s, 1H) 8.22 (s, 2H) 7.79 (s, 1H) 7.73 (s, 1H) 7.71 (s, 1H) 7.57 (d, J=8.10 Hz, 1H) 7.31 (m, 2H) 7.71-7.24 (m, 4H) 4.32 (m, 2H) 4.08 (br. s., 2H) 4.02 (br. s., 3H) 3.82 (s, 2H) 3.14 (br. s., 4H) 2.94 (br. s., 1H); MS (ESI) m/z [M+H]⁺ calcd. for C₂₇H₂₉N₈O, 481.3; found, 481.2; HPLC purity: 210 nm: 100%; 254 nm: 100%.

### Example 59: Synthesis of Exemplary Compound 56

### 6-(1-Methyl-1H-pyrazol-4-yl)-3-[4-(5-phenyl-4,5-dihydro-1,3-oxazol-2-yl)piperazin-1-yl]pyrazolo[1,5-a]pyridine (Compound 56)

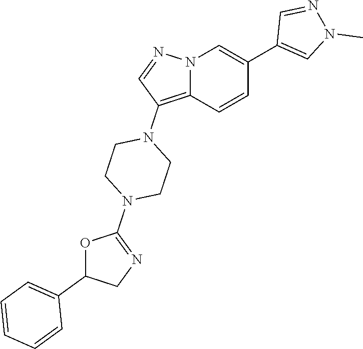 [SMILES: Cn1cc(-c2ccc3c(N4CCN(C5=NCC(c6ccccc6)O5)CC4)cnn3c2)cn1]

6-(1-Methyl-1H-pyrazol-4-yl)-3-piperazin-1-ylpyrazolo[1,5-a]pyridine (Compound 12, 0.10 g, 0.35 mmol) and 2-methoxy-5-phenyl-4,5-dihydro-1,3-oxazole (Compound S23, 0.31 g, 1.7 mmol) were taken up in a mixture of toluene (2 mL) and NMP (1 mL) in a round bottom flask under argon. N,N-Diisopropylethylamine (0.31 mL, 1.8 mmol) was added and the reaction mixture heated at 60° C. for 30 min. The reaction mixture was cooled to rt and the toluene was removed in vacuo to afford a dark brown oil. The oil was purified by Biotage Isolera chromatography using an Agela 40 g normal phase SG column eluting with a gradient of 95:5 to 1:1 DCM:MeOH. The desired fractions were combined and the solvent removed in vacuo affording a dark yellow/green oil. The oil was then purified by Isotec CombiFlash chromatography using a RediSepR_{f} Gold 15.5 g HP C18(aq) RP column eluting with a gradient from 0:100 to 3:2 ACN:water (NO TFA modifier). The desired fractions were combined, most of the ACN removed in vacuo and the remaining material lyophilized to dryness affording Compound 56 (1 mg, 1% yield) as an off-white powder. ¹H NMR (400 MHZ, CDCl₃) δ (ppm) 8.48 (s, 1H), 7.75 (d, J=7.34 Hz, 2H), 7.63 (s, 1H), 7.54 (d, J=8.68 Hz, 1H), 7.31-7.46 (m, 5H), 7.16 (dd, J=1.41, 9.11 Hz, 1H), 5.51-5.63 (m, 1H), 4.26 (dd, J=9.29, 12.47 Hz, 1H), 3.94-4.03 (m, 3H), 3.78 (dd, J=7.70, 12.47 Hz, 1H), 3.64-3.73 (m, 4H), 3.03-3.20 (m, 4H); MS (ESI) m/z [M+H]⁺ calcd. for C₂₄H₂₅N₇O: 427.2. Found: 428.1; HPLC purity: 210 nm: 90.7%; 254 nm: 91.3%.

### Example 60: Synthesis of Exemplary Compound 57

### (1R)-1-cyclohexylethyl 4-[6-(1-methyl-1H-pyrazol-4-yl)pyrazolo[1,5-a]pyridin-3-yl]piperazine-1-carboxylate (Compound 57)

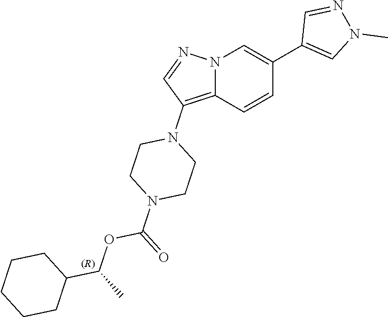

Compound 57 was prepared as described for the preparation of 1-ethylpropyl 4-[6-(1-methyl-1H-pyrazol-4-yl)pyrazolo[1,5-a]pyridin-3-yl]piperazine-1-carboxylate (Example 19, Compound 16) using (1R)-1-cyclohexylethanol (450 mg, 3.51 mmol) instead of 3-pentanol to afford 643 mg (65%) of (1R)-1-cyclohexylethyl 4-[6-(1-methyl-1H-pyrazol-4-yl)pyrazolo[1,5-a]pyridin-3-yl]piperazine-1-carboxylate (Compound 57) as a solid: ¹H NMR (400 MHZ, CDCl₃) δ (ppm) 8.47 (s, 1H) 7.75 (s, 1H) 7.72 (s, 1H), 7.62 (s, 1H) 7.53 (br. s. 1H) 7.16 (d, J=9.3 Hz, 1H) 4.70 (q, J=6.3 Hz, 1H) 3.98 (s, 3H) 3.70 (br. s., 4H) 3.05 (br. s., 4H) 1.75 (m, 5H) 1.49 (m, 1H) 1.22 (m 7H) 1.05 (m, 2H). MS (ESI) m/z [M+H]⁺ calcd. for C₂₄H₃₃N₆O₂: 437.3, Found: 437.2; HPLC purity: 210 nm: 100.0%; 254 nm: 100.0%.

### Example 61: Synthesis of Exemplary Compound 58

### 3-[4-(4-benzyl-1,3-oxazol-2-yl)piperazin-1-yl]-6-(1-methyl-1H-pyrazol-4-yl)pyrazolo[1,5-a]pyridine (Compound 58)

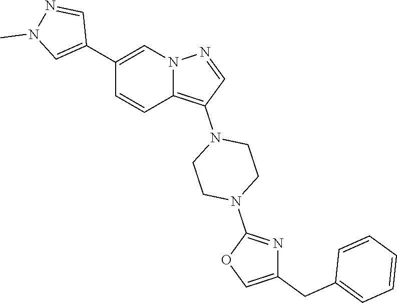 [SMILES: Cn1cc(-c2ccc3c(N4CCN(c5nc(Cc6ccccc6)co5)CC4)cnn3c2)cn1]

To a vial containing 3-bromo-6-(1-methyl-1H-pyrazol-4-yl)pyrazolo[1,5-a]pyridine (Compound S9, 0.24 g, 0.86 mmol) and was added a solution of 1-(4-benzyl-1,3-oxazol-2-yl])piperazine (Compound S24, 0.22 g, 0.91 mmol) dissolved in tert-butyl alcohol (2.1 mL) and 1,4-dioxane (0.54 mL). The solution was sparged with argon for 10 min. To the mixture was added sodium tert-butoxide (99 mg, 1.0 mmol). The mixture was sparged again for 5 min with argon and tBuXPhos Pd G1 (89 mg, 0.13 mmol) added. The reaction mixture was sparged with argon for 2 min with sonication and an additional 3 min with stirring. The vial was placed in a 55° C. oil bath and the reaction stirred for 16.5 h, cooled to ambient temperature then re-dosed with tBuXPhos Pd G1 (89 mg, 0.13 mmol). The reaction was stirred at 55° C. for another 3.5 h, cooled to room temperature and filtered through a pad of Celite rinsing the pad with DCM (20 mL). The filtrate was diluted with additional DCM (10 mL) and then washed with satd. aq. NaHCO₃ (15 mL). The aqueous layer was extracted with DCM (2×15 mL). The combined DCM layers were washed with brine (15 mL), dried (Na₂SO₄) filtered and concentrated. The residue was purified by ISOC chromatography (24 g normal phase cartridge) eluting with a gradient of 0-100% acetone in DCM to afford 99 mg that was a mixture of two compounds. This material was further purified by ISOC chromatography (50 g reverse phase cartridge) eluting with a gradient of 0-60% ACN in water to afford 69 mg (18%) of 3-[4-(4-benzyl-1,3-oxazol-2-yl)piperazin-1-yl]-6-(1-methyl-1H-pyrazol-4-yl [1,5-a]pyridine (Compound 58) as a yellow solid: ¹H NMR (400 MHZ, CDCl₃) δ (ppm) 8.47 (s, 1H) 7.75 (s, 1H) 7.73 (s, 1H) 7.61 (s, 1H) 7.52 (dd, J=9.17, 0.61 Hz, 1H) 7.29-7.35 (m, 4H) 7.21-7.27 (m, 1H) 7.15 (dd, J=9.17, 1.47 Hz, 1H) 6.80 (t, J=1.22 Hz, 1H) 3.98 (s, 3H) 3.78 (d, J=0.98 Hz, 2H) 3.66-3.73 (m, 4H) 3.10-3.17 (m, 4H); MS (ESI) m/z [M+H]⁺ calcd. for C₂₅H₂₆N₇O, 440.2; found, 440.2; HPLC purity: 210 nm: 65.3%; 254 nm: 66.8%.

### Example 62: Synthesis of Exemplary Compound 59

### (2-{4-[6-(1-methyl-1H-pyrazol-4-yl)pyrazolo[1,5-a]pyridin-3-yl]piperazin-1-yl}pyrimidin-5-yl) (pyridin-2-yl)methanol (Compound 59)

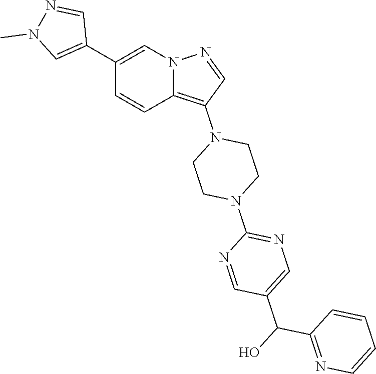 [SMILES: Cn1cc(-c2ccc3c(N4CCN(c5ncc(C(O)c6ccccn6)cn5)CC4)cnn3c2)cn1]

A mixture of 6-(1-methyl-1H-pyrazol-4-yl)-3-piperazin-1-ylpyrazolo[1,5-a]pyridine dihydrochloride (Compound S19, 0.401 g, 1.13 mmol), (2-chloropyrimidin-5-yl) (pyridin-2-yl)methanol (Compound S25, 0.300 g, 1.35 mmol) and potassium carbonate (0.779 g, 5.64 mmol) in 10 ml of DMF was sparged with N₂ for 15 min, then heated in an 40° C. heating block overnight. LCMS showed a mixture of (2-{4-[6-(1- methyl-1H-pyrazol-4-yl)pyrazolo[1,5-a]pyridin-3-yl]piperazin-1-yl}pyrimidin-5-yl) (pyridin-2-yl)methanol, 6-(1-methyl-1H-pyrazol-4-yl)-3-piperazin-1-ylpyrazolo[1,5-a]pyridine dihydrochloride and (2-{4-[6-(1-methyl-1H-pyrazol-4-yl)pyrazolo[1,5-a]pyridin-3-yl]piperazin-1-yl}pyrimidin-5-yl) (pyridin-2-yl)methanone. The mixture was cooled to rt and poured into 50 ml of water. The organic products were extracted into CH₂Cl₂ (3×50 ml). The combined organic extracts were washed with water (3×50 ml) and dried over Na₂SO₄. Evaporation of the solvent gave 3 g of a green oil containing DMF. The oil was taken up in 50 ml of EtOAc and extracted with 3×50 ml of water. Drying over Na₂SO₄ and evaporation gave 0.4 g of a dark oily solid. Chromatography on a Isolera® in 40% acetone/CH₂Cl₂ for 15 CV followed by 5% MeOH/CH₂Cl₂ for 10 CV followed by 10% MeOH/CH₂Cl₂ for 5 CV gave 0.06 g of (2-{4-[6-(1-methyl-1H-pyrazol-4-yl)pyrazolo[1,5-a]pyridin-3-yl]piperazin-1-yl}pyrimidin-5-yl) (pyridin-2-yl)methanol (Compound 59) as a yellow foam. NMR showed 4% CH₂Cl₂ by weight. The solid was taken up in 5 ml of MeOH and the solution was evaporated to dryness. The remaining foam was dried under hi-vac at rt over P₂O₅ for 24 h to give 0.050 g of (2-{4-[6-(1-methyl-1H-pyrazol-4-yl)pyrazolo[1,5-a]pyridin-3-yl]piperazin-1-yl}pyrimidin-5-yl) (pyridin-2-yl) methanol (Compound 59) as a foam. ¹H NMR (300 MHz, CDCl₃) δ (ppm) 8.61 (d, J=4.75 Hz, 1H), 8.48 (s, 1H), 8.33 (s, 2H), 7.57-7.79 (m, 5H), 7.15-7.31 (m, 4H), 5.68 (s, 1H), 4.09 (br. s., 4H), 3.98 (s, 3H), 3.16 (br. s., 4H); MS (ESI) m/z [M+H]⁺ calcd. for C₂₅H₂₅N₉O: 468.5; found: 468.1; HPLC purity: 210 nm; 100.0%; 254 nm: 100.0%.

### Example 63: Synthesis of Exemplary Compound 60

### 3-[1-(5-benzylpyrimidin-2-yl)-1,2,3,6-tetrahydropyridin-4-yl]-6-(1-methyl-1H-pyrazol-4-yl)pyrazolo[1,5-a]pyridine (Compound 60)

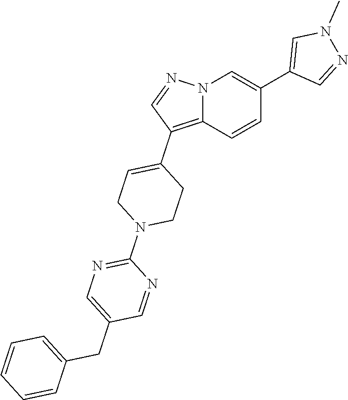 [SMILES: Cn1cc(-c2ccc3c(C4=CCN(c5ncc(Cc6ccccc6)cn5)CC4)cnn3c2)cn1]

Compound 60 was prepared as described for the preparation of 3-[1-(5-benzylpyrimidin-2-yl)-2,5-dihydro-1H-pyrrol-3-yl]-6-(1-methyl-1H-pyrazol-4-yl)pyrazolo[1,5-a]pyridine (Example 54, Compound 51) using [1-tert-butoxycarbonyl)-1,2,3,6-tetrahydropyridin-4-yl]boronic acid (295 mg, 1.30 mmol) to afford 135 mg (19%) of 3-[1-(5-benzylpyrimidin-2-yl)-1,2,3,6-tetrahydropyridin-4-yl]-6-(1-methyl-1H-pyrazol-4-yl)pyrazolo[1,5-a]pyridine (Compound 60) as a solid: ¹H NMR (400 MHZ, CDCl₃) δ (ppm) 8.56 (s, 1H) 8.23 (s, 2H) 7.95 (s, 1H) 7.76 (s, 1H) 7.65 (s, 1H) 7.74 (s, 1H) 7.71 (s, 1H) 7.63 (s 1H) 7.18 to 7.32 (m 5H) 6.11 (dt, J=3.1, 1.7x (2) Hz, 1H) 4.43 (q, J=2.6x (3) Hz, 2H) 4.11 (t, J=5.7x (2) Hz, 2H) 3.98 (s, 3H) 3.82 (s, 2H); MS (ESI) m/z [M+H]⁺ calcd. for C₂₇H₂₆N₇: 448.5; found, 448.1; HPLC purity: 210 nm: 98.7%; 254 nm: 98.6%.

### Example 64: Synthesis of Exemplary Compound 61

### cyclohexyl(2-{4-[6-(1-methyl-1H-pyrazol-4-yl)pyrazolo[1,5-a]pyridin-3-yl]piperazin-1-yl}pyrimidin-5-yl)methanol (Compound 61)

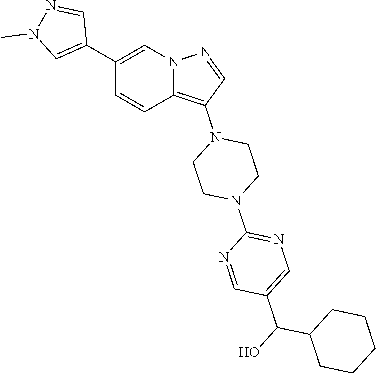 [SMILES: Cn1cc(-c2ccc3c(N4CCN(c5ncc(C(O)C6CCCCC6)cn5)CC4)cnn3c2)cn1]

To a suspension of cyclohexyl(2-{4-[6-(1-methyl-1H-pyrazol-4-yl)pyrazolo[1,5-a]pyridin-3-yl]piperazin-1-yl}pyrimidin-5-yl)methanone (Compound S26, 0.090 g, 0.19 mmol) in methanol (1.5 mL) (stirred for 1 min with solid NaOH) was added sodium tetrahydroborate. The mixture was stirred at rt overnight. HPLC (Agilent XDB C18 50×4.6 mm 1.8 micron column, Solvent A-Water (0.1% TFA) Solvent B-Acetonitrile (0.07% TFA). Gradient-5 min 95% A to 95% B; 1.0 min hold; then recycle. UV Detection @ 210 and 254 nm.) showed no starting material. The reaction was quenched by addition of 5 ml of saturated NH₄Cl and stirring at rt for 10 min. The organic solvents are evaporated on the rotovap and the remaining suspension was extracted with 3×10 ml of CH₂Cl₂. Drying over Na₂SO₄ and evaporation of the solvent gave 0.1 g of a yellow oil. Chromatography on an Isolera® in 3% MeOH/CH₂Cl₂ for 10 CV followed by 6% MeOH/CH₂Cl₂ for 6 CV on 4 g of silica gel gave 0.043 g (48%) of cyclohexyl(2-{4-[6-(1-methyl-1H-pyrazol-4-yl)pyrazolo[1,5-a]pyridin-3-yl]piperazin-1-yl}pyrimidin-5-yl)methanol (Compound 61) as a white solid. ¹H NMR (300 MHz, CDCl₃) δ (ppm) 8.47 (s, 1H), 8.30 (s, 2H), 7.74 (d, J=8.94 Hz, 2H), 7.55-7.63 (m, 2H), 7.16 (dd, J=9.17, 1.44 Hz, 1H), 4.29 (dd, J=7.26, 3.07 Hz, 1H), 4.05 (m, 4H), 3.97 (s, 3H), 3.11-3.18 (m, 4H), 2.02 (d, J=12.48 Hz, 1H), 1.74-1.89 (m, 2H), 1.58-1.74 (m, 3H), 1.51 (d, J=12.48 Hz, 1H), 1.11-1.33 (m, 3H), 0.83-1.11 (m, 2H); MS (ESI) m/z [M+H]⁺ calcd. for C₂₆H₃₂N₈O: 473.6; found: 473.2; HPLC purity: 210 nm; 100.0%; 254 nm: 100.0%.

### Example 65: Synthesis of Exemplary Compound 62

### 3-{4-[5-(cyclohexylmethyl)pyrimidin-2-yl]piperazin-1-yl}-6-(1-methyl-1H-pyrazol-4-yl)pyrazolo[1,5-a]pyridine (Compound 62)

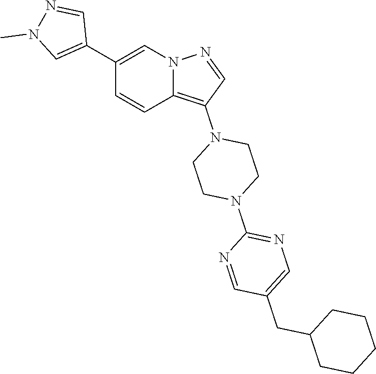 [SMILES: Cn1cc(-c2ccc3c(N4CCN(c5ncc(CC6CCCCC6)cn5)CC4)cnn3c2)cn1]

Trifluoroacetic acid (0.05380 mL, 0.6983 mmol) was added to a mixture of cyclohexyl(2-{4-[6-(1-methyl-1H-pyrazol-4-yl)pyrazolo[1,5-a]pyridin-3-yl]piperazin-1-yl}pyrimidin-5-yl)methanol (Compound 61, 0.033 g, 0.070 mmol) and triethylsilane (0.01673 mL, 0.1047 mmol) in 5 ml of CH₂Cl₂ at rt. The mixture was stirred at rt under N₂ for 1 hr. HPLC (Agilent XDB C18 50×4.6 mm 1.8 micron column, Solvent A-Water (0.1% TFA) Solvent B-Acetonitrile (0.07% TFA). Gradient-5 min 95% A to 95% B; 1.0 min hold; then recycle. UV Detection @ 210 and 254 nm.) showed 62% starting material at 210 nm. The mixture was stirred at RT overnight. HPLC showed no starting material. The mixture was diluted with 10 ml of CH₂Cl₂ and extracted with 3×10 ml of sat′d NaHCO₃. Drying over Na₂SO₄ and evaporation gave 0.034 g of an oil. Chromatography on an Isolera® in 5% MeOH/CH₂Cl₂ on 4 g of silica gel gave 0.016 g (50%) of 3-{4-[5-(cyclohexylmethyl)pyrimidin-2-yl]piperazin-1-yl}-6-(1-methyl-1H-pyrazol-4-yl)pyrazolo[1,5-a]pyridine (Compound 62) as an off white solid. ¹H NMR (300 MHz, CDCl₃) δ (ppm) 8.46-8.51 (m, 1H), 8.16 (s, 2H), 7.71-7.80 (m, 2H), 7.54-7.65 (m, 2H), 7.15 (dd, J=9.22, 1.49 Hz, 1H), 3.96-4.15 (m, 7H), 3.11-3.22 (m, 4H), 2.33 (d, J=6.98 Hz, 2H), 1.64-1.77 (m, 5H), 1.11-1.43 (m, 4H), 0.96 (br. s., 2H); MS (ESI) m/z [M+H]⁺ calcd. for C₂₆H₃₂N₈, 457.6; found, 457.3; HPLC purity: 210 nm: 100.0%; 254 nm: 100.0%.

### Example 66: Synthesis of Exemplary Compound 63

### 3-[1-(5-benzylpyrimidin-2-yl)piperidin-4-yl]-6-(1-methyl-1H-pyrazol-4-yl)pyrazolo[1,5-a]pyridine (Compound 63)

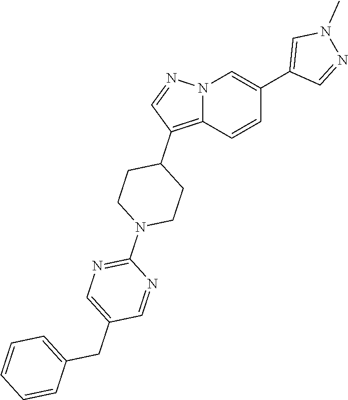 [SMILES: Cn1cc(-c2ccc3c(C4CCN(c5ncc(Cc6ccccc6)cn5)CC4)cnn3c2)cn1]

To a solution of 3-[1-(5-benzylpyrimidin-2-yl)-1,2,3,6-tetrahydropyridin-4-yl]-6-(1-methyl-1H-pyrazol-4-yl)pyrazolo[1,5-a]pyridine (Compound 60, 76 mg, 0.17 mmol) in DMF (6.2 mL) was added 10% palladium on carbon (124 mg, 50% water wet). The reaction flask was evacuated and filled with hydrogen gas three times and the reaction was stirred under an atmosphere of hydrogen. After 16 hr the reaction mixture was filtered through a pad of celite, washing the pad with EtOAc. The filtrate was washed with H₂O, dried with Na₂SO₄, filtered, and concentrated to yield 64 mg solid. The reaction mixture was diluted with DCM and was purified by Biotage chromatography (4 g normal phase cartridge) eluting with a gradient of 0-90% acetone in DCM to afford 47 mg (62%) of 3-[1-(5-benzylpyrimidin-2-yl)piperidin-4-yl]-6-(1-methyl-1H-pyrazol-4-yl)pyrazolo[1,5-a]pyridine (Compound 63) as a solid: ¹H NMR (400 MHZ, CDCl₃) δ (ppm) 8.55 (s, 1H) 8.20 (s, 2H) 7.77 (s, 1H) 7.76 (s, 1H) 7.62 (s, 1H) 7.61 (s, 1H) 7.54 (s, 1H) 7.51 (s, 1H) 7.31 (m, 2H) 7.21 (m, 4H) 4.89 (d J=13.3 Hz 2H) 3.98 (s, 3H) 3.81 (s, 2H) 3.07 (m, 3H) 2.06 (d, J=11.4 Hz, 2H) 1.81 (qd, J=12.6× (3), 4.1 Hz, 2H); MS (ESI) m/z [M+H]⁺ calcd. for C₂₇H₂₈N₇: 450.6; found, 450.2; HPLC purity: 210 nm: 100%; 254 nm: 100%.

### Example 67: Synthesis of Exemplary Compound 64

### (1S)-2,2,2-trifluoro-1-phenylethyl 4-[6-(1-methyl-1H-pyrazol-4-yl)pyrazolo[1,5-a]pyridin-3-yl]piperazine-1-carboxylate (Compound 64)

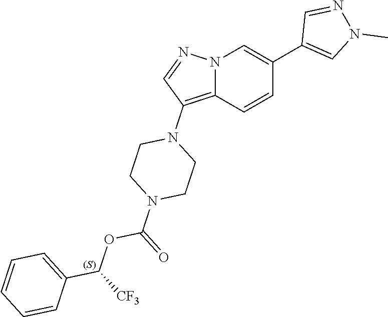

Compound 64 was prepared as described for the preparation of 1-ethylpropyl 4-[6-(1-methyl-1H-pyrazol-4-yl)pyrazolo[1,5-a]pyridin-3-yl]piperazine-1-carboxylate (Example 19, Compound 16) using (1S)-2,2,2-trifluoro-1-phenylethanol (130 mg, 0.75 mmol) to afford 232 mg (91%) of (1S)-2,2,2-trifluoro-1-phenylethyl 4-[6-(1-methyl-1H-pyrazol-4-yl)pyrazolo[1,5-a]pyridin-3-yl]piperazine-1-carboxylate (Compound 64) as a solid: ¹H NMR (400 MHZ, CDCl₃) δ (ppm) 8.47 (s, 1H) 7.75 (s, 1H) 7.72 (s, 1H), 7.62 (s, 1H) 7.55 to 7.46 (m, 3H) 7.46 to 7.39 (m, 3H) 7.17 (dd, J=9.2, 1.2 Hz, 1H) 6.15 (q, J=7 Hz, 1H) 3.98 (s, 3H) 3.79 (br. d., 4H) 3.08 (br. s., 4H); MS (ESI) m/z [M+H]⁺ calcd. for C₂₄H₂₄F₃N₆O₂: 485.2, Found: 485.1; HPLC purity: 210 nm: 100.0%; 254 nm: 100.0%.

### Example 68: Synthesis of Exemplary Compound 65

### 3-{4-[5-(4-chlorobenzyl)pyrimidin-2-yl]piperazin-1-yl}-6-(1-methyl-1H-pyrazol-4-yl)pyrazolo[1,5-a]pyridine (Compound 65)

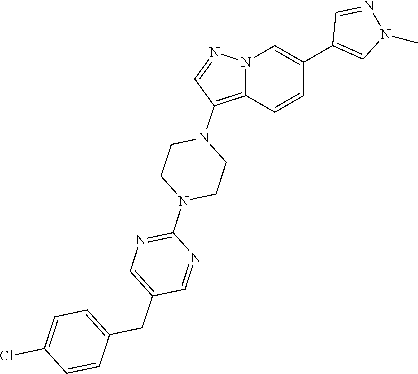 [SMILES: Cn1cc(-c2ccc3c(N4CCN(c5ncc(Cc6ccc(Cl)cc6)cn5)CC4)cnn3c2)cn1]

A vial containing 6-(1-methyl-1H-pyrazol-4-yl)-3-piperazin-1-ylpyrazolo[1,5-a]pyridine hydrochloride (Compound S19, 0.19 g, 80% w/w, 0.47 mmol) and 2-chloro-5-(4-chlorobenzyl)pyrimidine (Compound S27, 0.17 g, 0.71 mmol) and DMF (2 mL) was warmed to 76° C. Added K₂CO₃ (0.32 g, 2.3 mmol) and DMF (2 mL) to the reaction and warmed to 100° C. while under an argon atmosphere. After 5 h, the heat was removed, and the reaction allowed to stand overnight. The reaction was filtered through a plug of Celite. The filter pad was washed with DCM and the filtrate concentrated. The crude was dissolved in DCM (60 mL) and washed with aq. LiCl (5×20 mL). The DCM layer was dried (Na₂SO₄) filtered and concentrated. The residue was purified by ISCO chromatography (40 g normal phase cartridge) eluting with a gradient of 0-100% EtOAc in hexanes followed by 10% MeOH in EtOAc to afford 0.14 g, (60%) of 3-{4-[5-(4-chlorobenzyl)pyrimidin-2-yl]piperazin-1-yl}-6-(1-methyl-1H-pyrazol-4-yl)pyrazolo[1,5-a]pyridine (Compound 65) as a grey solid: ¹H NMR (400 MHZ, DMSO-d₆) δ (ppm) 8.81 (s, 1H) 8.31 (s, 2H) 8.22 (s, 1H) 7.97 (s, 1H) 7.77 (s, 1H) 7.68 (d, J=8.80 Hz, 1H) 7.30-7.38 (m, 3H) 7.24-7.30 (m, 2H) 3.84-3.93 (m, 7H) 3.79 (s, 2H) 2.99-3.08 (m, 4H); MS (ESI) m/z [M+H]⁺ calcd. for C₂₆H₂₆ClN₈, 485.2; found, 485.1; HPLC purity: 210 nm: 100%; 254 nm: 100%.

### Example 69: Synthesis of Exemplary Compound 66

### 3-{4-[5-(4-fluorobenzyl)pyrimidin-2-yl]piperazin-1-yl}-6-(1-methyl-1H-pyrazol-4-yl)pyrazolo[1,5-a]pyridine (Compound 66)

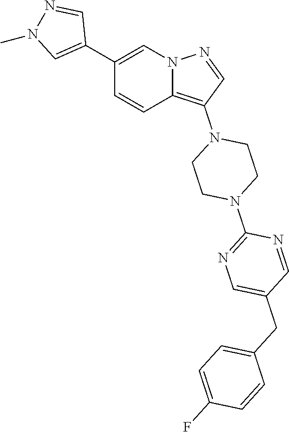 [SMILES: Cn1cc(-c2ccc3c(N4CCN(c5ncc(Cc6ccc(F)cc6)cn5)CC4)cnn3c2)cn1]

A vial containing 6-(1-methyl-1H-pyrazol-4-yl)-3-piperazin-1-ylpyrazolo[1,5-a]pyridine hydrochloride (Compound S19, 0.20 g, 0.63 mmol) and 2-chloro-5-(4-fluorobenzyl)pyrimidine (Compound S28, 0.21 g, 0.71 mmol) and DMF (3 mL) was warmed to 70° C. Added K₂CO₃ (0.43 g, 3.1 mmol) to the reaction and warmed to 100° C. while under an argon atmosphere overnight. The reaction was cooled to room temperature and filtered through a plug of Celite. The filter pad was washed with DCM (45 mL) and the filtrate concentrated. The crude was dissolved in DCM (30 mL) and washed with aq. LiCl (5×20 mL). The DCM layer was dried (Na₂SO₄) filtered and concentrated. The residue was purified by ISCO chromatography (40 g normal phase cartridge) eluting with a gradient of 0-100% EtOAc in hexanes followed by 10% MeOH in EtOAc to afford 0.15 g, (51%) of 3-{4-[5-(4-fluorobenzyl)pyrimidin-2-yl]piperazin-1-yl}-6-(1-methyl-1H-pyrazol-4-yl)pyrazolo[1,5-a]pyridine (Compound 66) as a pale yellow solid: ¹H NMR (400 MHZ, CDCl₃) δ (ppm) 8.47 (s, 1H) 8.19 (s, 2H) 7.75 (s, 1H) 7.72 (s, 1H) 7.61 (s, 1H) 7.56 (d, J=9.17 Hz, 1H) 7.11-7.18 (m, 3H) 6.96-7.04 (m, 2H) 3.99-4.04 (m, 4H) 3.98 (s, 3H) 3.79 (s, 2H) 3.09-3.16 (m, 4H); MS (ESI) m/z [M+H]⁺ calcd. for C₂₆H₂₆FN₈, 469.2; found, 469.2; HPLC purity: 210 nm: 100%; 254 nm: 100%.

### Example 70: Synthesis of Exemplary Compound 67

### 3-{4-[5-(4-methylbenzyl)pyrimidin-2-yl]piperazin-1-yl}-6-(1-methyl-1H-pyrazol-4-yl)pyrazolo[1,5-a]pyridine (Compound 67)

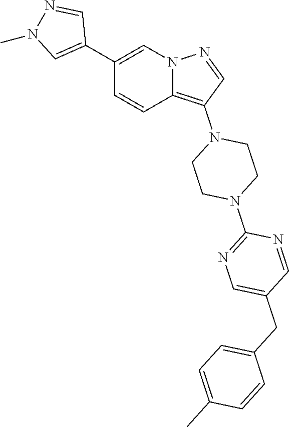 [SMILES: Cc1ccc(Cc2cnc(N3CCN(c4cnn5cc(-c6cnn(C)c6)ccc45)CC3)nc2)cc1]

A vial containing 6-(1-methyl-1H-pyrazol-4-yl)-3-piperazin-1-ylpyrazolo[1,5-a]pyridine hydrochloride (Compound S19, 0.15 g, 0.47 mmol) and 2-chloro-5-(4-methylbenzyl)pyrimidine (Compound S29, 0.15 g, 0.71 mmol) and DMF (2 mL) was warmed to 100° C. Added K₂CO₃ (0.32 g, 2.3 mmol) to the reaction and warmed to 100° C. while under an argon atmosphere. After 7 h, the heat was removed, and the reaction allowed to stand overnight. The reaction was filtered through a plug of Celite. The filter pad was washed with DCM and the filtrate concentrated. The crude was dissolved in DCM (60 mL) and washed with aq. LiCl (5×20 mL). The DCM layer was dried (Na₂SO₄) filtered and concentrated. The residue was purified by ISCO chromatography (40 g normal phase cartridge) eluting with a gradient of 0-100% EtOAc in hexanes followed by 10% MeOH in EtOAc to afford 0.12 g, (56%) of 3-{4-[5-(4-methylbenzyl)pyrimidin-2-yl]piperazin-1-yl}-6-(1-methyl-1H-pyrazol-4-yl)pyrazolo[1,5-a]pyridine (Compound 67) as a yellow solid: ¹H NMR (400 MHZ, CDCl₃) δ (ppm) 8.47 (s, 1H) 8.21 (s, 2H) 7.75 (s, 1H) 7.72 (s, 1H) 7.60-7.64 (m, 1H) 7.56 (dd, J=9.17, 0.73 Hz, 1H) 7.02-7.20 (m, 5H) 3.94-4.07 (m, 7H) 3.78 (s, 2H) 3.06-3.21 (m, 4H) 2.33 (s, 3H); MS (ESI) m/z [M+H]⁺ calcd. for C₂₇H₂₉N₈, 465.3; found, 465.2; HPLC purity: 210 nm: 100%; 254 nm: 100%.

### Example 71: Synthesis of Exemplary Compound 68

### 6-(1-methyl-1H-pyrazol-4-yl)-3-{4-[5-(tetrahydro-2H-pyran-4-ylmethyl)pyrimidin-2-yl]piperazin-1-yl}pyrazolo[1,5-a]pyridine (Compound 68)

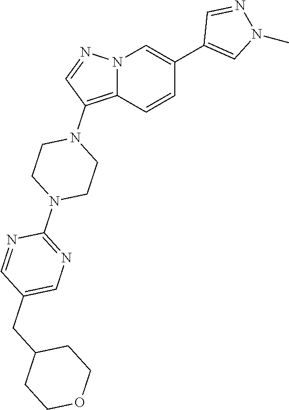 [SMILES: Cn1cc(-c2ccc3c(N4CCN(c5ncc(CC6CCOCC6)cn5)CC4)cnn3c2)cn1]

To a vial containing 3-bromo-6-(1-methyl-1H-pyrazol-4-yl)pyrazolo[1,5-a]pyridine (Compound S9, 83 mg, 0.30 mmol) and 2-piperazin-1-yl-5-(tetrahydro-2H-pyran-4-ylmethyl)pyrimidine (Compound S30, 94 mg, 0.36 mmol) was added tert-butyl alcohol (2.2 mL) and 1,4-dioxane (0.62 mL). The partially dissolved mixture was sparged with argon for 5 min. To the mixture was added sodium tert-butoxide (43 mg, 0.45 mmol). The mixture was sparged again for 5 min with argon and tBuXPhos Pd G1 (31 mg, 0.05 mmol) added. The reaction mixture was sparged with argon for 5 min and an additional 2 min with sonication. The flask was heated in a 55° C. oil bath. After stirring 20 h, the reaction cooled to ambient temperature and diluted with EtOAc (10 mL). The mixture was washed with saturated aq. NaHCO₃ (10 mL). The aq. layer was washed with EtOAc (3×10 mL). The combined EtOAc layers were dried (Na₂SO₄), filtered and concentrated. The residue was purified by ISOC chromatography (40 g normal phase cartridge) eluting with a gradient of 0-85% acetone in DCM to afford 27 mg, (20%) of 6-(1-methyl-1H-pyrazol-4-yl)-3-{4-[5-(tetrahydro-2H-pyran-4-ylmethyl)pyrimidin-2-yl]piperazin-1-yl}pyrazolo[1,5-a]pyridine (Compound 68) as a white solid: ¹H NMR (400 MHZ, CDCl₃) δ (ppm) 8.47 (s, 1H) 8.17 (s, 2H) 7.75 (s, 1H) 7.72 (s, 1H) 7.62 (s, 1H) 7.56 (d, J=9.66 Hz, 1H) 7.15 (dd, J=9.17, 1.34 Hz, 1H) 3.93-4.04 (m, 9H) 3.34 (td, J=11.77, 1.90 Hz, 2H) 3.10-3.18 (m, 4H) 2.39 (d, J=7.09 Hz, 2H) 1.55-1.73 (m, 3H) 1.33 (qd, J=12.25, 4.46 Hz, 2H); MS (ESI) m/z [M+H]⁺ calcd. for C₂₆H₃₁N₈O, 459.3; found, 459.3; HPLC purity: 210 nm: 98.8%; 254 nm: 100.0%.

### Example 72: Synthesis of Exemplary Compound 69

### (2-{4-[6-(1-methyl-1H-pyrazol-4-yl)pyrazolo[1,5-a]pyridin-3-yl]piperazin-1-yl}pyrimidin-5-yl)(tetrahydro-2H-pyran-4-yl)methanol (Compound 69)

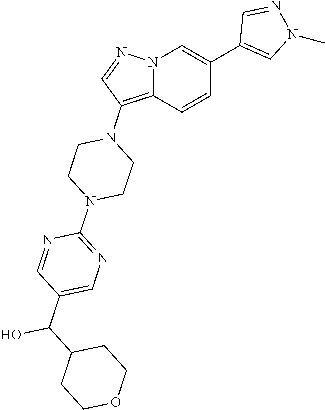 [SMILES: Cn1cc(-c2ccc3c(N4CCN(c5ncc(C(O)C6CCOCC6)cn5)CC4)cnn3c2)cn1]

To a suspension of (2-{4-[6-(1-methyl-1H-pyrazol-4-yl)pyrazolo[1,5-a]pyridin-3-yl]piperazin-1-yl}pyrimidin-5-yl)(tetrahydro-2H-pyran-4-yl)methanone (Compound S31, 0.147 g, 0.93 mmol) in MeOH (2.5 mL) was added sodium tetrahydroborate (24 mg, 0.63 mmol). After 18 h, additional sodium tetrahydroborate (24 mg, 0.63 mmol) was added and the reaction stirred for another 3 h at room temperature. The reaction was cooled in an ice bath and quenched with saturated aq. NH₄Cl. The reaction was warmed to room temperature, stirred for 30 min., and diluted with satd. aq. NaHCO₃(pH=9). The reaction was extracted with DCM (×3), dried (Na₂SO₄), filtered and concentrated. The residue was purified by ISCO chromatography (24 g normal phase cartridge) eluting with a gradient of ethyl acetate in hexanes followed by a second chromatography (20 g normal phase cartridge) using a gradient of 0-100% acetone in DCM to afford 69 mg (47%) of (2-{4-[6-(1-methyl-1H-pyrazol-4-yl)pyrazolo[1,5-a]pyridin-3-yl]piperazin-1-yl}pyrimidin-5-yl)(tetrahydro-2H-pyran-4-yl) methanol (Compound 69) as a white solid: ¹H NMR (400 MHZ, CDCl₃) δ (ppm) 8.47 (s, 1H) 8.30 (s, 2H) 7.75 (s, 1H) 7.71 (s, 1H) 7.62 (s, 1H) 7.56 (dd, J=9.17, 0.61 Hz, 1H) 7.16 (dd, J=9.17, 1.47 Hz, 1H) 4.29 (dd, J=7.76, 2.63 Hz, 1H) 4.01-4.10 (m, 5H) 3.91-4.01 (m, 4H) 3.28-3.45 (m, 2H) 3.08-3.18 (m, 4H) 1.80-2.00 (m, 3H) 1.45 (qd, J=12.21, 4.46 Hz, 1H) 1.25-1.35 (m, 2H); MS (ESI) m/z [M+H]⁺ calcd. for C₂₅H₃₁N₈O₂, 475.3; found, 475.3; HPLC purity: 210 nm: 99.5%; 254 nm: 99.6%.

### Example 73: Synthesis of Exemplary Compound 70

### 3-[4-(2-benzylpyrimidin-5-yl)piperazin-1-yl]-6-(1-methyl-1H-pyrazol-4-yl)pyrazolo[1,5-a]pyridine (Compound 70)

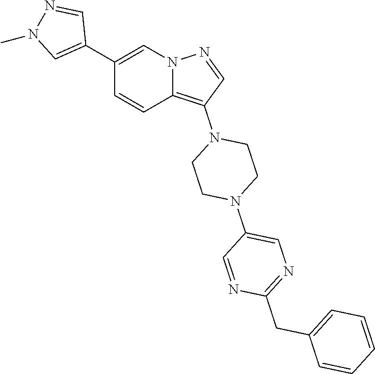 [SMILES: Cn1cc(-c2ccc3c(N4CCN(c5cnc(Cc6ccccc6)nc5)CC4)cnn3c2)cn1]

A flask containing 3-bromo-6-(1-methyl-1H-pyrazol-4-yl)pyrazolo[1,5-a]pyridine (Compound S9, 0.082 g, 0.30 mmol) and 2-benzyl-5-piperazin-1-ylpyrimidine (Compound S32, 0.090 g, 0.35 mmol) was treated with tert-butyl alcohol (0.817 mL, 8.54 mmol) and 1,4-dioxane (0.41 mL, 5.2 mmol). The solution was sparged with argon gas for 15 min. The solution was treated with tBuXPhos Pd G1 (0.030 g, 0.04 mmol) and sodium tert-butoxide (0.034 g, 0.35 mmol) and the mixture was sparged again for 10 min with argon gas then heated in a 55° C. oil bath overnight. LCMS showed no starting material. The mixture was cooled to rt and the catalyst was removed by filtration through Celite 545R. The filter cake was rinsed with 30 ml of CH₂Cl₂. The filtrate was extracted with 2×20 ml of saturated NaHCO₃ and 20 ml of brine. Drying over Na₂SO₄ and evaporation gave 0.23 g of a dark oil. Chromatography on an Isolera® in 30% acetone/CH₂Cl₂ to 70% acetone/CH₂Cl₂ over 15 CV on 4 g of silica gel gave 0.052 g (39%) of 3-[4-(2-benzylpyrimidin-5-yl)piperazin-1-yl]-6-(1-methyl-1H-pyrazol-4-yl)pyrazolo[1,5-a]pyridine (Compound 70). ¹H NMR (300 MHZ, CDCl₃) δ (ppm) 8.49 (s, 1H), 8.40 (s, 2H), 7.76 (s, 2H), 7.63 (s, 1H), 7.53 (d, J=9.13 Hz, 1H), 7.29-7.41 (m, 4H), 7.13-7.27 (m, 2H), 4.25 (s, 2H), 3.99 (s, 3H), 3.36-3.47 (m, 4H), 3.17-3.30 (m, 4H); MS (ESI) m/z [M+H]⁺ calcd. for C₂₆H₂₇N₈: 451.5; found: 451.2; HPLC purity: 210 nm; 98.0%; 254 nm: 100.0%.

### Example 74: Synthesis of Exemplary Compound 71

### (2-{4-[6-(1-Methyl-1H-pyrazol-4-yl)pyrazolo[1,5-a]pyridin-3-yl]piperazin-1-yl}pyrimidin-5-yl)(phenyl)methanol (Compound 71)

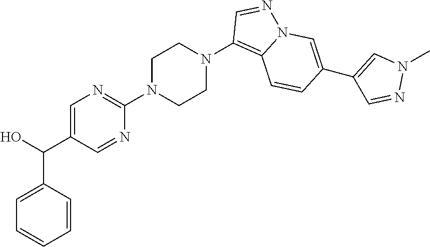 [SMILES: Cn1cc(-c2ccc3c(N4CCN(c5ncc(C(O)c6ccccc6)cn5)CC4)cnn3c2)cn1]

(2-{4-[6-(1-Methyl-1H-pyrazol-4-yl)pyrazolo[1,5-a]pyridin-3-yl]piperazin-1-yl}pyrimidin-5-yl)(phenyl)methanone (Compound S33, 0.140 g, 0.30 mmol) was slurried in a mixture of MeOH (2 mL) and THF (5 mL) at rt under argon. Sodium borohydride (37 mg, 0.98 mmol) was added in one portion (with potential for gas evolution). After 30 minutes at rt, the reaction mixture was cooled to 0° C., and the reaction mixture quenched by the addition of saturated aq. NH₄Cl (1 mL) (with potential for gas evolution). The reaction mixture was warmed to rt and stirred for 1 hour. Water (5 mL) was added causing dissolution of any solids and the mixture was poured into a separatory funnel. The aqueous layer was adjusted to pH ˜4 by the addition of 2M citric acid. The organic layer was extracted with EtOAc (25 mL×2). The combined organic fractions were dried over sodium sulfate, filtered and the solvent removed in vacuo to afford a yellow solid. The material was dissolved in 0.6 mL of DMF and purified by Isotec CombiFlash chromatography using a RediSepR_{f} Gold 15.5 g HP C18(aq) RP column eluting with a gradient from 1:4 to 7:3 ACN:water (NO added TFA modifier). The desired fractions were combined, most of the ACN removed in vacuo and the remaining aqueous layer lyophilized to dryness affording 32 mg (23% yield) of (2-{4-[6-(1-Methyl-1H-pyrazol-4-yl)pyrazolo[1,5-a]pyridin-3-yl]piperazin-1-yl}pyrimidin-5-yl)(phenyl)methanol (Compound 71) as a faint yellow powder. ¹H NMR (400 MHz, DMSO-d₆) δ (ppm) 8.82 (s, 1H), 8.32 (s, 2H), 8.23 (s, 1H), 7.97 (s, 1H), 7.77 (s, 1H), 7.68 (d, J=9.29 Hz, 1H), 7.38-7.43 (m, 2H), 7.30-7.37 (m, 3H), 7.21-7.28 (m, 1H), 5.92 (d, J=4.03 Hz, 1H), 5.66 (d, J=4.03 Hz, 1H), 3.88-3.93 (m, 4H), 3.87 (s, 3H), 2.93-3.10 (m, 4H); MS (ESI) m/z [M+H]⁺ calcd. for C₂₆H₂₇N₈O: 467.2. Found: 467.2; HPLC purity: 210 nm: 100%; 254 nm: 100%.

### Example 75: Synthesis of Exemplary Compound 72

### 3-[3-(5-benzylpyrimidin-2-yl) azetidin-1-yl]-6-(1-methyl-1H-pyrazol-4-yl)pyrazolo[1,5-a]pyridine (Compound 72)

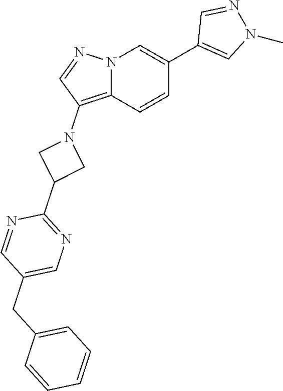 [SMILES: Cn1cc(-c2ccc3c(N4CC(c5ncc(Cc6ccccc6)cn5)C4)cnn3c2)cn1]

To a vial containing 3-bromo-6-(1-methyl-1H-pyrazol-4-yl)pyrazolo[1,5-a]pyridine (Compound S9, 0.17 g, 0.62 mmol) and was added a solution of 2-azetidin-3-yl-5-benzylpyrimidine (Compound S34, 0.146 g, 0.65 mmol) dissolved in tert-butyl alcohol (1.5 mL) and 1,4-dioxane (0.38 mL). The solution was sparged with argon for 15 min. To the mixture was added sodium tert-butoxide (71 mg, 0.74 mmol). The mixture was sparged again for 5 min with argon and tBuXPhos Pd G1 (63 mg, 0.09 mmol) added. The reaction mixture was sparged with argon for 5 min and an additional 2 min with sonication. The flask was heated in a 55° C. oil bath for 17 h, cooled to ambient temperature and filtered through a pad of Ceilte rinsing the pad with DCM (20 mL). The filtrate was diluted with additional DCM (10 mL) and then washed with saturated aq. NaHCO₃ (15 mL) and brine (15 mL). The DCM layer was dried (Na₂SO₄) filtered and concentrated. The residue was purified by ISOC chromatography (20 g normal phase cartridge) eluting with a gradient of 0-100% DCM in acetone afford 142 mg, (55%) of 3-[4-(5-benzylpyrimidin-2-yl)piperazin-1-yl]-6-(1-methyl-1H-pyrazol-4-yl)pyrazolo[1,5-a]pyridine (Compound 72) as a yellow/tan solid: ¹H NMR (400 MHz, CDCl₃) δ (ppm) 8.57 (s, 2H) 8.39 (s, 1H) 7.72 (s, 1H) 7.58 (s, 1H) 7.53 (s, 1H) 7.44 (d, J=9.17 Hz, 1H) 7.30-7.36 (m, 2H) 7.22-7.29 (m, 1H) 7.18 (d, J=7.09 Hz, 2H) 6.99 (dd, J=9.23, 1.41 Hz, 1H) 4.35-4.44 (m, 2H) 4.24-4.34 (m, 3H) 3.96 (s, 5H); MS (ESI) m/z [M+H]⁺ calcd. for C₂₅H₂₄N₇, 422.2; found, 422.1; HPLC purity: 210 nm: 98.8%; 254 nm: 98.8%.

### Example 76: Synthesis of Exemplary Compound 73

### (1R)-1-Phenylethyl 4-[6-(1-methyl-1H-pyrazol-4-yl)pyrazolo[1,5-a]pyrimidin-3-yl]piperazine-1-carboxylate (Compound 73)

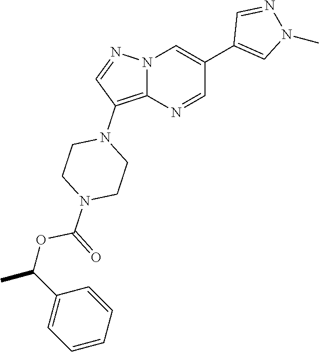 [SMILES: C[C@@H](OC(=O)N1CCN(c2cnn3cc(-c4cnn(C)c4)cnc23)CC1)c1ccccc1]

6-(1-Methyl-1H-pyrazol-4-yl)-3-piperazin-1-ylpyrazolo[1,5-a]pyrimidine (70 mg, 0.2 mmol) and 4-nitrophenyl (1R)-1-phenylethyl carbonate (89 mg, 0.3 mmol) was taken up in acetonitrile (3.5 mL) at rt under argon in a round-bottomed flask fitted with a magnetic stirrer. N,N-Diisopropylethylamine (0.3 mL, 1.5 mmol) was added to the orange/yellow suspension and stirred under argon for 18 hours. The reaction mixture was concentrated in vacuo affording a yellow semi-solid which was dissolved in 6 mL of DCM and was purified by Biotage Isolera chromatography using an Agela 40 g normal phase SG column eluting first with 1:1 EtOAc/hexanes and then 100% EtOAc. The desired fractions were combined and the solvent removed in vacuo to afford an orange oil. MTBE (5 mL) was added causing the oil to solidify. The solids were stirred at rt for 30 minutes and then collected by filtration affording 72 mg (68%) of (1R)-1-phenylethyl 4-[6-(1-methyl-1H-pyrazol-4-yl)pyrazolo[1,5-a]pyrimidin-3-yl]piperazine-1-carboxylate (Compound 73) as orange/yellow solids. ¹H NMR (400 MHZ, DMSO-d₆) δ (ppm) 9.19 (d, J=2.08 Hz, 1H), 8.64 (d, J=2.08 Hz, 1H), 8.30 (s, 1H), 8.04 (s, 1H), 7.90 (s, 1H), 7.34-7.43 (m, 4H), 7.25-7.34 (m, 1H), 5.75 (q, J=6.52 Hz, 1H), 3.89 (s, 3H), 3.64 (br. s., 4H), 3.23 (br. s., 4H), 1.49 (d, J=6.60 Hz, 3H); MS (ESI) m/z [M+H]⁺ calcd. for C₂₃H₂₅N₇O₂: 432.2. Found: 432.2; HPLC purity: 210 nm: 100%; 254 nm: 100%.

### Example 77: Synthesis of Exemplary Compound 74

### (4-Fluorophenyl)(2-{4-[6-(1-methyl-1H-pyrazol-4-yl)pyrazolo[1,5-a]pyrimidin-3-yl]piperazin-1-yl}pyrimidin-5-yl)methanol (Compound 74)

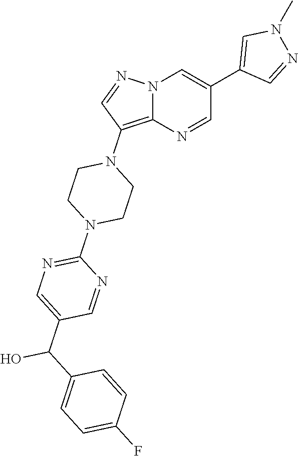 [SMILES: Cn1cc(-c2cnc3c(N4CCN(c5ncc(C(O)c6ccc(F)cc6)cn5)CC4)cnn3c2)cn1]

(4-Fluorophenyl)(2-{4-[6-(1-methyl-1H-pyrazol-4-yl)pyrazolo[1,5-a]pyrimidin-3-yl]piperazin-1-yl}pyrimidin-5-yl)methanone (Compound S35, 0.06 g, 0.12 mmol) was slurried in a mixture of MeOH (1 mL) and THF (2 mL) in a round bottom flask which had been flushed under Ar. To the slurry was added sodium borohydride (15 mg, 0.4 mmol) in one portion. The reaction was stirred at rt overnight. The reaction mixture was cooled to 0° C., and saturated aqueous NH₄Cl (0.4 mL) was added generating solids. The cooling bath was removed and the mixture stirred at rt for 1 hour. The solids were removed by filtration and washed with THF. The filtrate was concentrated in vacuo to give a reddish solid. The solids were dissolved in 1.5 mL of DMF and purified by Isotec CombiFlash chromatography using a RediSepR_{f} Gold 15.5 g HP C18(aq) RP column eluting with a gradient from 0:100 to 3:2 ACN:water (w/added TFA modifier). The desired fractions were combined and the ACN removed in vacuo. The residual turbid aqueous layer was transferred to a separatory funnel, neutralized to pH˜8 by the addition of saturated aqueous sodium bicarbonate. The aqueous layer was extracted with EtOAc (25 mL×2) and the combined organic fractions were dried over sodium sulfate, filtered and the solvent removed in vacuo to afford a yellow-orange solid. The solid was dried in vacuum oven at 50° C. overnight affording 25 mg (42%) of (4-fluorophenyl)(2-{4-[6-(1-methyl-1H-pyrazol-4-yl)pyrazolo[1,5-a]pyrimidin-3-yl]piperazin-1-yl}pyrimidin-5-yl)methanol (Compound 74) as a yellow-orange solid. ¹H NMR (400 MHZ, CDCl₃) δ (ppm) 8.58 (d, J=2.08 Hz, 1H), 8.47 (d, J=2.08 Hz, 1H), 8.31 (s, 2H), 7.80 (s, 1H), 7.78 (s, 1H), 7.68 (s, 1H), 7.39 (dd, J=5.38, 8.68 Hz, 2H), 7.08 (t, J=8.62 Hz, 2H), 5.77 (s, 1H), 4.05-4.14 (m, 4H), 4.01 (s, 3H), 3.27-3.48 (m, 4H), 2.25 (br. s., 1H); MS (ESI) m/z [M+H]⁺ calcd. for C₂₅H₂₄FN₉O: 485.2. Found: 486.2; HPLC purity: 210 nm: 100%; 254 nm: 100%.

### Example 78: Synthesis of Exemplary Compound 75

### 3-[1-(5-benzylpyrimidin-2-yl)pyrrolidin-3-yl]-6-(1-methyl-1H-pyrazol-4-yl)pyrazolo[1,5-a]pyridine (Compound 75)

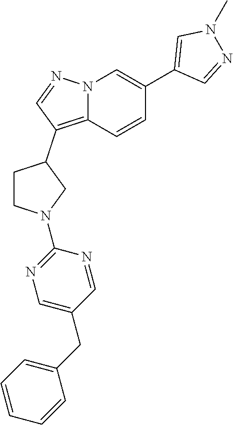 [SMILES: Cn1cc(-c2ccc3c(C4CCN(c5ncc(Cc6ccccc6)cn5)C4)cnn3c2)cn1]

To a solution of crude 3-[6-(1-methyl-1H-pyrazol-4-yl)pyrazolo[1,5-a]pyridin-3-yl]pyrrolidinium trifluoroacetate (Compound S36, 98 mg, 0.26 mmol; assumed) in N-methylpyrrolidinone (2.0 mL) was added 5-benzyl-2-chloropyrimidine (58 mg, 0.28 mmol) and potassium carbonate (142 mg, 1.03 mmol) and the mixture was heated at 110° C. After 1.25 h it was determined that the starting material was consumed, the reaction mixture was cooled partially and the solvent was removed under high vac, resulting in a greyish brown residue that was purified by flash chromatography (35 g silica gel; 40-50% acetone/CH₂Cl₂) resulting in a light yellow solid that was found to contain NMP. The solid was triturated with 1/1 MTBE/hex, allowing the solvent to stand over the solid overnight. The solids were isolated by filtration and dried to yield 71 mg (66%) of 3-[1-(5-benzylpyrimidin-2-yl)pyrrolidin-3-yl]-6-(1-methyl-1H-pyrazol-4-yl)pyrazolo[1,5-a]pyridine (Compound 75) as a light grey solid: 1H NMR (400 MHZ, CDCl₃) δ (ppm) 8.55 (s, 1H) 8.22 (s, 2H) 7.83 (s, 1H) 7.75 (d, J=0.61 Hz, 1H) 7.62 (s, 1H) 7.49-7.56 (m, 1H) 7.28-7.35 (m, 2H) 7.12-7.25 (m, 4H) 4.17 (dd, J=10.39, 7.09 Hz, 1H) 3.98 (s, 3H) 3.88 (ddd, J=11.13, 7.95, 3.30 Hz, 1H) 3.82 (s, 2H) 3.58-3.79 (m, 3H) 2.51 (dtd, J=12.35, 6.30, 6.30, 3.42 Hz, 1H) 2.13-2.30 (m, 1H); MS (ESI) m/z [M+H]⁺ calcd. for C₂₆H₂₄N₇: 436.2; found, 436.3; HPLC purity: 210 nm: 98.7%; 254 nm: 100%.

### Example 79: Synthesis of Exemplary Compound 76

### 3-[1-(5-benzylpyrimidin-2-yl)-1H-pyrrol-3-yl]-6-(1-methyl-1H-pyrazol-4-yl)pyrazolo[1,5-a]pyridine (Compound 76)

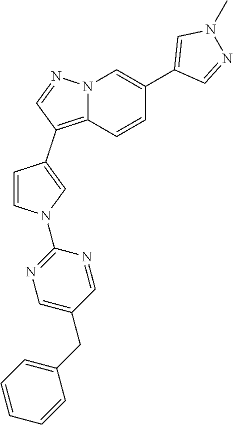 [SMILES: Cn1cc(-c2ccc3c(-c4ccn(-c5ncc(Cc6ccccc6)cn5)c4)cnn3c2)cn1]

A solution of 3-[1-(5-benzylpyrimidin-2-yl)-2,5-dihydro-1H-pyrrol-3-yl]-6-(1-methyl-1H-pyrazol-4-yl)pyrazolo[1,5-a]pyridine (Compound 51, 140 mg, 0.32 mmol) in CH₂Cl₂ (7 mL) and CHCl₃ (7 mL) was treated with MnO₂ (281 mg, 3.23 mmol). The mixture was sonicated for 1 min, followed by stirring at 40° C. overnight. The reaction mixture was filtered through a pad of celite, washing the pad with an additional 40 mL CHCl₃. The filtrate was concentrated to a brown glass that was purified by flash chromatography (30 g silica gel, 0-3% MeOH/CH₂Cl₂) to yield a yellowish orange solid that was found to contain an impurity. The solid (128 mg) was treated with MTBE (11 mL), sonicated for 2 min and the mixture was allowed to stand at RT overnight. The mixture was filtered to yield 95 mg (69%) of 3-[1-(5-benzylpyrimidin-2-yl)-1H-pyrrol-3-yl]-6-(1-methyl-1H-pyrazol-4-yl)pyrazolo[1,5-a]pyridine (Compound 76) as a tan solid: ¹H NMR (400 MHZ, CDCl₃) δ (ppm) 8.58 (s, 1H) 8.49 (s, 2H) 8.11 (s, 1H) 8.00 (t, J=1.83 Hz, 1H) 7.85-7.89 (m, 1H) 7.82 (d, J=9.29 Hz, 1H) 7.77 (s, 1H) 7.64 (s, 1H) 7.31-7.40 (m, 2H) 7.24-7.31 (m, 2H) 7.22 (d, J=7.21 Hz, 2H) 6.65 (dd, J=3.12, 1.77 Hz, 1H) 3.99 (s, 5H); MS (ESI) m/z [M+H]⁺ calcd. for C₂₆H₂₂N₇: 432.2; found, 432.2; HPLC purity: 210 nm: 97.9%; 254 nm: 100%.

### Example 80: Synthesis of Exemplary Compound 77

### (1R)-1-(4-Fluorophenyl)ethyl 4-[6-(1-methyl-1H-pyrazol-4-yl)pyrazolo[1,5-a]pyridin-3-yl]piperazine-1-carboxylate (Compound 77)

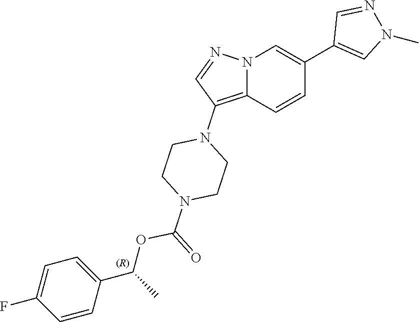

Compound 77 was prepared as described for the preparation of 1-ethylpropyl 4-[6-(1-methyl-1H-pyrazol-4-yl)pyrazolo[1,5-a]pyridin-3-yl]piperazine-1-carboxylate (Example 19, Compound 16) using (1R)-1-(4-fluorophenyl)ethanol (87 mg, 0.62 mmol) to afford 113 mg (70%) of (1R)-1-(4-fluorophenyl)ethyl 4-[6-(1-methyl-1H-pyrazol-4-yl)pyrazolo[1,5-a]pyridin-3-yl]piperazine-1-carboxylate (Compound 77) as a beige solid: ¹H NMR (400 MHZ, CDCl₃) δ (ppm) 8.47 (s, 1H) 7.74 (s, 1H) 7.70 (s, 1H), 7.61 (s, 1H) 7.51 (d, J=8.8 Hz, 1H) 7.35 (m, 2H) 7.15 (dd, J=9.2, 1.2 Hz, 1H) 7.05 (m, 2H) 5.84 (q, J=6.6x (3) Hz, 1H), 3.98 (s, 3H) 3.71 (br. s., 4H) 3.03 (br.s., 4H) 1.57 (d, J=6.6 Hz, 3H); ¹⁹F NMR (400 MHz, CDCl₃) d ppm −114.7; MS (ESI) m/z [M+H]⁺ calcd. for C₂₄H₂₆FN₆O₂: 449.2, Found: 449.2; HPLC purity: 210 nm: 99.4%; 254 nm: 99.1%.

### Example 81: Synthesis of Exemplary Compound 78

### (1R)-1-(4-Chlorophenyl)ethyl 4-[6-(1-methyl-1H-pyrazol-4-yl)pyrazolo[1,5-a]pyridin-3-yl]piperazine-1-carboxylate (Compound 78)

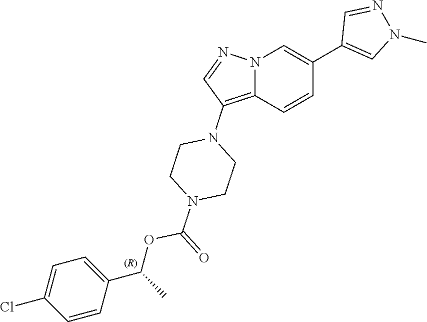

Compound 78 was prepared as described for the preparation of 1-ethylpropyl 4-[6-(1-methyl-1H-pyrazol-4-yl)pyrazolo[1,5-a]pyridin-3-yl]piperazine-1-carboxylate (Example 19, Compound 16) using (1R)-1-(4-chlorophenyl)ethanol (97 mg, 0.62 mmol) to afford 121 mg (72%) of (1R)-1-(4-chlorophenyl)ethyl 4-[6-(1-methyl-1H-pyrazol-4-yl)pyrazolo[1,5-a]pyridin-3-yl]piperazine-1-carboxylate (Compound 78) as a solid: ¹H NMR (400 MHZ, CDCl₃) δ (ppm) 8.47 (s, 1H) 7.75 (d, J=0.5 Hz, 1H) 7.71 (s, 1H), 7.62 (s, 1H) 7.52 (d, J=8.8 Hz, 1H) 7.33 (m, 4H) 7.16 (d, J=9.2 Hz, 1H) 5.83 (q, J=6.6 Hz, 1H), 3.98 (s, 3H) 3.71 (br. s., 4H) 3.04 (br.s., 4H) 1.56 (d, J=6.6 Hz, 3H); MS (ESI) m/z [M+H]⁺ calcd. for C₂₄H₂₆ClN₆O₂: 465.2, Found: 465.2; HPLC purity: 210 nm: 100.0%; 254 nm: 100.0%.

### Example 82: Synthesis of Exemplary Compound 79

### (1R)-1-(3-Fluorophenyl)ethyl 4-[6-(1-methyl-1H-pyrazol-4-yl)pyrazolo[1,5-a]pyridin-3-yl]piperazine-1-carboxylate (Compound 79)

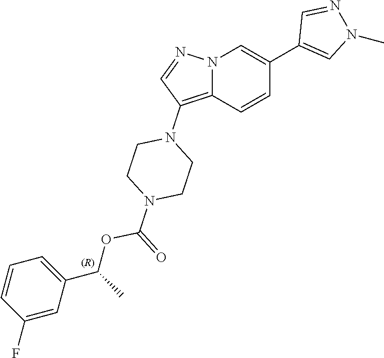

Compound 79 was prepared as described for the preparation of 1-ethylpropyl 4-[6-(1-methyl-1H-pyrazol-4-yl)pyrazolo[1,5-a]pyridin-3-yl]piperazine-1-carboxylate (Example 19, Compound 16) using (1R)-1-(3-fluorophenyl)ethanol (88 mg, 0.63 mmol) to afford 116 mg (72%) of (1R)-1-(3-fluorophenyl)ethyl 4-[6-(1-methyl-1H-pyrazol-4-yl)pyrazolo[1,5-a]pyridin-3-yl]piperazine-1-carboxylate (Compound 79) as a solid: ¹H NMR (400 MHZ, CDCl₃) δ (ppm) 8.47 (s, 1H) 7.75 (s, 1H) 7.71 (s, 1H), 7.62 (s, 1H) 7.52 (d, J=8.6 Hz, 1H) 7.33 (td, J=7.9, 5.9 Hz, 1H) 7.15 (m, 2H) 7.07 (dt, J=9.8, 1.9 Hz, 1H) 6.99 (m, 1H) 5.85 (q, J=6.7 Hz, 1H), 3.98 (s, 3H) 3.73 (br. s., 4H) 3.05 (br. s., 4H) 1.57 (d, J=6.6 Hz, 3H); ¹⁹F NMR (400 MHz, CDCl₃) d ppm −112.7; MS (ESI) m/z [M+H]⁺ calcd. for C₂₄H₂₆FN₆O₂: 449.2, Found: 449.2; HPLC purity: 210 nm: 100.0%; 254 nm: 100.0%.

### Example 83: Synthesis of Exemplary Compound 80

### (1R)-1-(Phenyl)propyl 4-[6-(1-methyl-1H-pyrazol-4-yl)pyrazolo[1,5-a]pyridin-3-yl]piperazine-1-carboxylate (Compound 80)

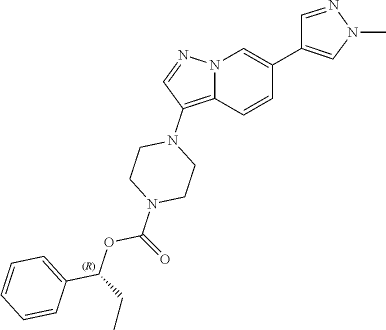

Compound 80 was prepared as described for the preparation of 1-ethylpropyl 4-[6-(1-methyl-1H-pyrazol-4-yl)pyrazolo[1,5-a]pyridin-3-yl]piperazine-1-carboxylate (Example 19, Compound 16) using (1R)-1-phenylpropan-1-ol (86 mg, 0.63 mmol) to afford 105 mg (66%) of (1R)-1-phenylpropyl 4-[6-(1-methyl-1H-pyrazol-4-yl)pyrazolo[1,5-a]pyridin-3-yl]piperazine-1-carboxylate (Compound 80) as a solid: ¹H NMR (400 MHZ, CDCl₃) δ (ppm) 8.47 (s, 1H) 7.75 (s, 1H) 7.71 (s, 1H), 7.61 (s, 1H) 7.52 (d, J=8.7 Hz, 1H) 7.36 (m, 4H) 7.30 (m, 1H) 7.15 (d, J=9, 1H) 7.07 (dt, J=9.8, 1.9 Hz, 1H) 6.99 (m, 1H) 5.65 (t, J=6.8 Hz, 1H), 3.98 (s, 3H) 3.77 (br. s., 4H) 3.04 (br. s., 4H) 1.92 (m, 1H) 0.93 (t, J=7.4 Hz, 3H); MS (ESI) m/z [M+H]⁺ calcd. for C₂₅H₂₉N₆O₂: 445.2, Found: 445.2; HPLC purity: 210 nm: 99.4%; 254 nm: 99.2%.

### Example 84: Synthesis of Exemplary Compound 81

### (1R)-1-(2-Fluorophenyl)ethyl 4-[6-(1-methyl-1H-pyrazol-4-yl)pyrazolo[1,5-a]pyridin-3-yl]piperazine-1-carboxylate (Compound 81)

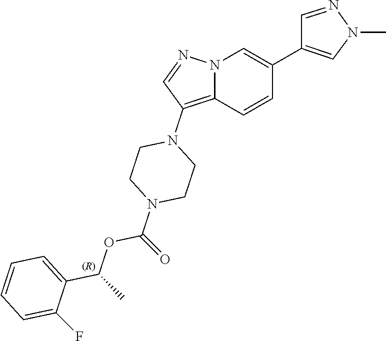

Compound 81 was prepared as described for the preparation of 1-ethylpropyl 4-[6-(1-methyl-1H-pyrazol-4-yl)pyrazolo[1,5-a]pyridin-3-yl]piperazine-1-carboxylate (Example 19, Compound 16) using (1R)-1-(2-fluorophenyl)ethanol (88 mg, 0.63 mmol) to afford 109 mg (68%) of (1R)-1-(2-fluorophenyl)ethyl 4-[6-(1-methyl-1H-pyrazol-4-yl)pyrazolo[1,5-a]pyridin-3-yl]piperazine-1-carboxylate (Compound 81) as a solid: ¹H NMR (400 MHZ, CDCl₃) δ (ppm) 8.47 (s, 1H) 7.75 (s, 1H) 7.71 (s, 1H), 7.61 (s, 1H) 7.50 (d, J=9.2 Hz, 1H) 7.39 (td, J=7.5, 1.2 Hz, 1H) 7.28 (m, 1H) 7.16 (m, 2H) 7.06 (m, 1H) 6.11 (q, J=6.5 Hz, 1H), 3.98 (s, 3H) 3.73 (br. s., 4H) 3.04 (br. s., 4H) 1.60 (d, J=6.6 Hz, 3H); 19F NMR (400 MHZ, CDCl₃) d ppm −118.3; MS (ESI) m/z [M+H]⁺ calcd. for C₂₄H₂₆FN₆O₂: 449.2, Found: 449.2; HPLC purity: 210 nm: 99.1%; 254 nm: 98.8%.

### Example 85: Synthesis of Exemplary Compound 82

### 3-[4-(4-benzylphenyl)piperazin-1-yl]-6-(1-methyl-1H-pyrazol-4-yl)pyrazolo[1,5-a]pyridine-D₇ (Compound 83)

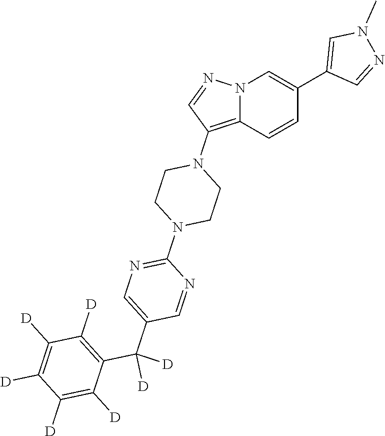 [SMILES: [2H]c1c([2H])c([2H])c(C([2H])([2H])c2cnc(N3CCN(c4cnn5cc(-c6cnn(C)c6)ccc45)CC3)nc2)c([2H])c1[2H]]

To a solution of 6-(1-methyl-1H-pyrazol-4-yl)-3-piperazin-1-ylpyrazolo[1,5-a]pyridine (Compound 12, 400.0 mg, 1.42 mmol) in N-methylpyrrolidinone (8.3 mL) was added 5-benzyl-2-chloropyrimidine-D₇ (Compound S38, 330 mg, 1.56 mmol) and potassium carbonate (780 mg, 5.7 mmol) and the mixture was heated at 110° C. After 5.5 h it was determined that the starting material was consumed, the reaction mixture was cooled to RT, diluted with 70 mL EtOAc, washed with 50 mL portions of H₂O and brine and dried over MgSO₄. The solution was filtered and concentrated to a residue that was purified by flash chromatography (80 g silica gel; 20-40% acetone/CH₂Cl₂) resulting in a light green solid that was triturated with Et₂O. The solid was filtered and dried to yield 426 mg (70%) of 3-[4-(4-benzylphenyl)piperazin-1-yl]-6-(1-methyl-1H-pyrazol-4-yl)pyrazolo[1,5-a]pyridine-D₇ (Compound 82) as a light green solid: ¹H NMR (400 MHZ, CDCl₃) δ (ppm) 8.47 (s, 1H) 8.22 (s, 2H) 7.75 (s, 1H) 7.72 (s, 1H) 7.62 (s, 1H) 7.56 (d, J=9.17 Hz, 1H) 7.15 (dd, J=9.17, 1.47 Hz, 1H) 3.99-4.04 (m, 4H) 3.98 (s, 3H) 3.10-3.17 (m, 4H); MS (ESI) m/z [M+H]⁺ calcd. for C₂₆H₁₈D₇N₇: 458.3; found, 458.3; HPLC purity: 210 nm: 100%; 254 nm: 100%.

### Example 86: Synthesis of Exemplary Compound 83

### tert-butyl 4-(7-(1-methyl-1H-pyrazol-4-yl)imidazo[1,2-c]pyrimidin-3-yl)piperazine-1-carboxylate (Compound 83)

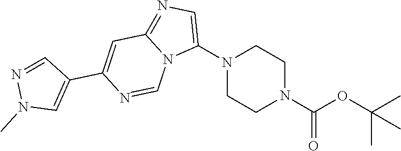 [SMILES: Cn1cc(-c2cc3ncc(N4CCN(C(=O)OC(C)(C)C)CC4)n3cn2)cn1]

To a solution of 6-(1-methyl-1H-pyrazol-4-yl)pyrimidin-4-amine (Compound S97) (200 mg, 1.14 mmol) and di-tert-butyl 4,4′-(1,2-bis(1H-benzo[d][1,2,3]triazol-1-yl)ethane-1,2-diyl)bis(piperazine-1-carboxylate) (Compound S47) (722 mg, 1.14 mmol) in DCM (25 mL) was added ZnBr₂ (257 mg, 1.14 mmol) at 25° C. under nitrogen. The mixture was stirred at reflux for 4 h under nitrogen. The solution was filtered, and the filtrate was collected. The filtrate was poured into 2N NaOH (10 mL) and extracted with 10% MeOH in DCM (3×20 mL). The combined organic layer was washed with water (20 mL) and brine (20 mL), dried over Na₂SO₄, and filtered. The filtrate was concentrated under reduced pressure. The residue was purified by prep-TLC (EtOAc:MeOH=10:1) to afford 38 mg (8%) of tert-butyl 4-(7-(1-methyl-1H-pyrazol-4-yl)imidazo[1,2-c]pyrimidin-3-yl)piperazine-1-carboxylate (Compound 83) as a yellow solid. ¹H NMR (400 MHZ, CDCl₃) δ (ppm) 8.82 (s, 1H), 7.96 (s, 1H), 7.90 (s, 1H), 7.47 (s, 1H), 7.20 (s, 1H), 3.97 (s, 3H), 3.66-3.56 (m, 4H), 3.05-3.03 (m, 4H), 1.50 (s, 9H). MS (ESI) m/z [M+H]⁺ calcd. for C₁₉H₂₅N₇O₂, 384.2; Found, 384.1. HPLC purity: 220 nm: 95.6%.

Using the procedures described in Example 86, Compound 83 and reagents, starting materials, and conditions known to those skilled in the art were used to prepare the compounds shown in Table A.

TABLE A

| Compound | Name | Characterization |
|---|---|---|
| Compound 84 | tert-butyl 4-(7-(pyridin-3- | ¹H NMR (400 MHz, CDCl₃) δ (ppm) 9.31 |
| | yl)imidazo[1,2-c]pyrimidin-3- | (d, J = 1.6 Hz, 1H), 8.99 (s, 1H), 8.66 (s, |
| | yl)piperazine-1-carboxylate | 1H), 8.33~8.35 (m, 1H), 7.93 (s, 1H), |
| | | 7.47 - 7.49 (m, 1H), 7.33 - 7.45 (m, 1H), |
| | | 3.68 (t, J = 4.8 Hz, 4H), 3.09 (t, J = 4.8 |
| | | Hz, 4H), 1.51 (s, 9H). MS (ESI) m/z |
| | | [M + H]⁺ calcd. for C₂₀H₂₄N₆O₂, 381.2; |
| | | Found, 381.1. HPLC purity: 220 nm: |
| | | 98.2%. |
| Compound 85 | tert-butyl 4-(7-(1-methyl-1H- | ¹H NMR (400 MHz, CDCl₃) δ (ppm) 8.81 |
| | pyrazol-4-yl)imidazo[1,2- | (s, 1H), 7.96 (s, 1H), 7.90 (s, 1H), 7.46 (s, |
| | c]pyrimidin-3-yl)-1,4- | 1H), 7.20 (d, J = 2.4 Hz, 1H), 3.97 (s, |
| | diazepane-1-carboxylate | 3H), 3.65-3.57 (m, 4H), 3.33-3.21 (m, |
| | | 4H), 2.05-1.93 (m, 2H), 1.51 (s, 9H). |
| | | MS (ESI) m/z [M + H]⁺ calcd. for |
| | | C₂₀H₂₇N₇O₂, 398.2; Found, 398.0. HPLC |
| | | purity: 220 nm: 96.0%. |
| Compound 86 | tert-butyl 4-(7-(1-(2- | ¹H NMR (400 MHz, CDCl₃) δ (ppm) 8.84 |
| | methoxyethyl)-1H-pyrazol-4- | (s, 1H), 8.02 (s, 1H), 8.00 (s, 1H), 7.52 (s, |
| | yl)imidazo[1,2-c]pyrimidin-3- | 1H), 7.20 (s, 1H), 4.29-4.36 (m, 2H), |
| | yl)-1,4-diazepane-1- | 3.89-3.92 (m, 2H), 3.59-3.62 (m, 4H), |
| | carboxylate | 3.39 (s, 3H), 3.21-3.29 (m, 4H), 1.94-1.96 |
| | | (m, 2H), 1.29-1.25 (m, 9H). MS |
| | | (ESI) m/z [M + H]⁺ calcd. for C₂₀H₂₇N₇O₂, |
| | | 442.3; Found, 442.1. HPLC purity: 220 |
| | | nm: 95.1%. |
| Compound 87 | tert-butyl 4-(7-(1-(2- | ¹H NMR (400 MHz, DMSO-d₆) δ (ppm) |
| | methoxyethyl)-1H-pyrazol-4- | 8.59 (d, J = 7.2 Hz, 1H), 8.50 (s, 1H), |
| | yl)imidazo[1,2-c]pyrimidin-3- | 8.14 (s, 1H), 7.32 (d, J = 7.2 Hz, 1H), |
| | yl)-1,4-diazepane-1- | 7.30 (s, 1H), 3.92 (s, 3H), 3.56-3.52 (m, |
| | carboxylate | 4H), 2.96-2.93 (m, 4H), 1.44 (s, 9H). |
| | | MS (ESI) m/z [M + H]⁺ calcd. for |
| | | C₁₉H₂₅N₇O₂, 384.3; Found, 384.3. HPLC |
| | | purity: 220 nm: 97.5%. |

### Example 87: Synthesis of Exemplary Compound 88

### tert-butyl 4-(7-(1-(2-methoxyethyl)-1H-pyrazol-4-yl)imidazo[1,2-c]pyrimidin-3-yl)piperazine-1-carboxylate (Compound 88)

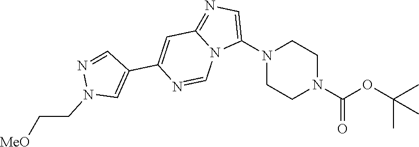 [SMILES: COCCn1cc(-c2cc3ncc(N4CCN(C(=O)OC(C)(C)C)CC4)n3cn2)cn1]

To a solution of 6-(1-(2-methoxyethyl)-1H-pyrazol-4-yl)pyrimidin-4-amine (Compound S39) (0.20 g, 0.90 mmol) and di-tert-butyl 4,4′-(1,2-bis(1H-benzo[d][1,2,3]triazol-1-yl)ethane-1,2-diyl)bis(piperazine-1-carboxylate) (Compound S47) (0.57 g, 0.90 mmol) in DCM (10 mL) was added ZnBr₂ (0.20 g, 0.90 mmol) at 25° C. under nitrogen. The mixture was stirred at 35° C. for 4 h. The mixture was poured into IN NaOH (20 mL) and extracted with DCM (30 mL×3). The combined organic layer was washed with brine (30 mL) and dried over Na₂SO₄, concentrated in vacuum to give the crude oil. The crude oil was purified by silica gel chromatography then further purified by prep-HPLC to afford 29.6 mg (11%) of tert-butyl 4-(7-(1-(2-methoxyethyl)-1H-pyrazol-4-yl)imidazo[1,2-c]pyrimidin-3-yl)piperazine-1-carboxylate (Compound 88) as a yellow solid. ¹H NMR (400 MHZ, CDCl₃) δ (ppm) 8.85 (s, 1H), 8.02 (d, J=8.8 Hz, 2H), 7.56 (s, 1H), 7.21 (s, 1H), 4.35 (d, J=5.2 Hz, 2H), 3.76-3.78 (m, 2H), 3.62˜3.64 (m, 4H), 3.35 (s, 3H), 3.03˜3.05 (m, 4H), 1.50 (s, 9H). MS (ESI) m/z [M+H]⁺ calcd. for C₂₁H₂₉N₇O₃, 428.2; Found, 428.1. HPLC purity: 220 nm: 95.6%.

Using the procedures described in Example 87, Compound 88 and reagents, starting materials, and conditions known to those skilled in the art were used to prepare the compounds shown in Table B.

TABLE B

| Compound | Name | Characterization |
|---|---|---|
| Compound 89 | tert-butyl 4-(7-(1-methyl-1H- | ¹H NMR (400 MHz, CDCl₃) δ (ppm) |
| | pyrazol-4-yl)imidazo[1,2- | 8.24-8.17 (m, 1H), 8.12 (s, 1H), 8.02 (s, 1H), |
| | a]pyrimidin-3-yl)-1,4- | 7.34 (d, J = 4.8 Hz, 1H), 7.03 (d, J = 7.2 |
| | diazepane-1-carboxylate | Hz, 1H), 3.98 (s, 1H), 3.52-3.48 (m, 4H), |
| | | 3.25-3.16 (m, 4H), 1.96-1.89 (m, 2H), |
| | | 1.51-1.45 (m, 9H). MS (ESI) m/z |
| | | [M + H]⁺ calcd. for C₂₀H₂₇N₇O₂, 398.2; |
| | | Found, 398.3. HPLC purity: 220 nm: |
| | | 98.1%. |

### Example 88: Synthesis of Exemplary Compound 90

### tert-butyl 4-(7-(tetrahydrofuran-3-yl)imidazo[1,2-c]pyrimidin-3-yl)piperazine-1-carboxylate (Compound 90)

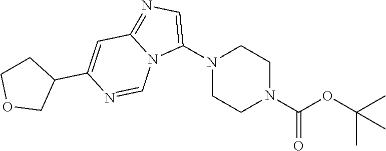 [SMILES: CC(C)(C)OC(=O)N1CCN(c2cnc3cc(C4CCOC4)ncn23)CC1]

To a solution of 6-(tetrahydrofuran-3-yl)pyrimidin-4-amine (Compound S41) (200 mg, 1.21 mmol) and di-tert-butyl 4,4′-(1,2-bis(1H-benzo[d][1,2,3]triazol-1-yl)ethane-1,2-diyl)bis(piperazine-1-carboxylate) (Compound S47) (1149 mg, 1.81 mmol) in ACN (30 mL) was added ZnBr₂ (817 mg, 3.63 mmol) at 25° C. under nitrogen. The mixture was stirred at reflux for 4 h under nitrogen. The solution was filtered and the filtrate was collected. The filtrate was poured into IN NaOH (50 mL) and extracted with DCM:MeOH=10:1 (20 mL×3). The combined organic layer was washed with water (20 mL) and brine (20 mL), dried over Na₂SO₄, and filtered. The filtrate was concentrated under reduced pressure. The residue was purified by prep-TLC (EtOAc:MeOH:NH₃·H₂O=200:20:1) to give a solid which was further purified by prep-HPLC to afford 23 mg (5%) of tert-butyl 4-(7-(tetrahydrofuran-3-yl)imidazo[1,2-c]pyrimidin-3-yl)piperazine-1-carboxylate (Compound 90) as a yellow solid. ¹H NMR (400 MHz, CDCl₃) δ (ppm) 8.80 (d, J=1.2 Hz, 1H), 7.16 (s, 1H), 6.81 (s, 1H), 4.17 (t, J=8.0 Hz, 1H), 4.11-4.06 (m, 1H), 3.97-3.94 (m, 1H), 3.91-3.87 (m, 1H), 3.61-3.58 (m, 4H), 3.53-3.48 (m, 1H), 3.32-3.29 (m, 4H), 2.39-2.30 (m, 1H), 2.25-2.16 (m, 1H), 1.51 (s, 9H). MS (ESI) m/z [M+H]⁺ calcd. for C₁₉H₂₇N₅O₃, 374.2; Found, 374.1. HPLC purity: 220 nm: 99.3%.

Using the procedures described in Example 88, Compound 90 and reagents, starting materials, and conditions known to those skilled in the art were used to prepare the compounds shown in Table C.

TABLE C

| Compound | Name | Characterization |
|---|---|---|
| Compound 91 | tert-butyl 4-(7-(4-methyl-1H- | ¹H NMR (400 MHz, CDCl₃) δ (ppm) 8.72 |
| | imidazol-1-yl)imidazo[1,2- | (d, J = 0.8 Hz, 1H), 8.27 (s, 1H), 7.22 (s, |
| | c]pyrimidin-3-yl)piperazine- | 1H), 7.14 (s, 1H), 6.86 (s, 1H), 3.58-3.61 |
| | 1-carboxylate | (m, 4H), 3.32-3.34 (m, 4H), 2.30 (s, 3H), |
| | | 1.49 (s, 9H). MS (ESI) m/z [M + H]⁺ |
| | | calcd. for C₁₉H₂₅N₇O₂, 384.2; Found, |
| | | 384.0. HPLC purity: 220 nm: 95.2%. |
| Compound 92 | tert-butyl 4-(7-(pyridin-3- | ¹H NMR (400 MHz, CDCl₃) δ (ppm) 9.41 |
| | yl)imidazo[1,2-c]pyrimidin-3- | (s, 1H), 9.01-9.03 (m, 1H), 8.71 (s, 1H), |
| | yl)-1,4-diazepane-1- | 8.50 (s, 1H), 8.14-8.16 (m, 1H), 7.58-7.62 |
| | carboxylate | (m, 1H), 7.37 (s, 1H), 3.64-3.69 (m, |
| | | 4H), 3.35-3.39 (m, 4H), 1.96-2.03 (m, |
| | | 2H), 1.53-1.51 (m, 9H). MS (ESI) m/z |
| | | [M + H]⁺ calcd. for C₂₁H₂₆N₆O₂, 395.2; |
| | | Found, 395.0. HPLC purity: 220 nm: |
| | | 96.7%. |
| Compound 93 | tert-butyl 4-(7-(4-methyl-1H- | ¹H NMR (400 MHz, DMSO-d₆) δ (ppm) |
| | imidazol-1-yl)imidazo[1,2- | 9.04 (s, 1H), 8.40 (s, 1H), 7.81 (s, 1H), |
| | c]pyrimidin-3-yl)-1,4- | 7.70 (s, 1H), 7.29 (s, 1H), 3.35-3.53 (m, |
| | diazepane-1-carboxylate | 4H), 3.20-3.30 (m, 4H), 2.18 (s, 3H), |
| | | 1.87-1.83 (m, 2H), 1.44-1.41 (m, 9H). |
| | | MS (ESI) m/z [M + H]⁺ calcd. for |
| | | C₂₀H₂₇N₇O₂, 398.2; Found, 398.1. HPLC |
| | | purity: 220 nm: 97.0%. |
| Compound 89 | tert-butyl 4-(7-(1-methyl-1H- | ¹H NMR (400 MHz, CDCl₃) δ (ppm) |
| | pyrazol-4-yl)imidazo[1,2- | 8.24-8.17 (m, 1H), 8.12 (s, 1H), 8.02 (s, 1H), |
| | alpyrimidin-3-yl)-1,4- | 7.34 (d, J = 4.8 Hz, 1H), 7.03 (d, J = 7.2 |
| | diazepane-1-carboxylate | Hz, 1H), 3.98 (s, 1H), 3.52-3.48 (m, 4H), |
| | | 3.25-3.16 (m, 4H), 1.96-1.89 (m, 2H), |
| | | 1.51-1.45 (m, 9H). MS (ESI) m/z |
| | | [M + H]⁺ calcd. for C₂₀H₂₇N₇O₂, 398.2; |
| | | Found, 398.3. HPLC purity: 220 nm: |
| | | 98.1%. |

### Example 89: Synthesis of Exemplary Compound 94

### tert-butyl 4-(7-(tetrahydrofuran-3-yl)imidazo[1,2-c]pyrimidin-3-yl)-1,4-diazepane-1-carboxylate (Compound 94)

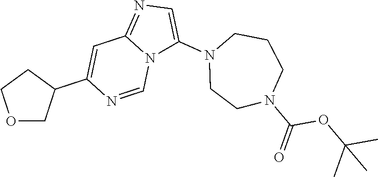 [SMILES: CC(C)(C)OC(=O)N1CCCN(c2cnc3cc(C4CCOC4)ncn23)CC1]

To a solution of 6-(tetrahydrofuran-3-yl)pyrimidin-4-amine (Compound S41) (0.30 g, 1.80 mmol) and di-tert-butyl 4,4′-(1,2-bis(1H-benzo[d][1,2,3]triazol-1-yl)ethane-1,2-diyl)bis(1,4-diazepane-1-carboxylate) (Compound S48) (1.19 g, 1.80 mmol) in ACN (20 mL) was added ZnBr₂ (0.41 g, 1.80 mmol) at 25° C. under nitrogen. The mixture was stirred at 80° C. for 6 h. The mixture was poured into IN NaOH (20 mL) and extracted with DCM (3×40 mL). The combined organic layer was washed with brine (20 mL) and dried over Na₂SO₄, concentrated in vacuum to give the crude oil. The crude oil was purified by silica gel chromatography to give the crude oil. The crude oil was purified by prep-HPLC to afford 40.0 mg (6%) of tert-butyl 4-(7-(tetrahydrofuran-3-yl)imidazo[1,2-c]pyrimidin-3-yl)-1,4-diazepane-1-carboxylate (Compound 94) as a yellow oil. ¹H NMR (400 MHZ, CDCl₃) δ (ppm) 8.89-8.92 (d, J=11.6 Hz, 1H), 7.44-7.47 (m, 1H), 7.25 (s, 1H), 4.09-4.19 (m, 2H), 3.91-3.99 (m, 2H), 3.57-3.64 (m, 5H), 3.26-3.32 (m, 4H), 2.26-2.35 (m, 1H), 2.21-2.25 (m, 1H), 1.95-1.15 (m, 2H), 1.53-1.52 (m, 9H). MS (ESI) m/z [M+H]⁺ calcd. for C₂₀H₂₉N₅O₃, 388.2; Found, 388.0. HPLC purity: 220 nm: 98.8%.

### Example 90: Synthesis of Exemplary Compound 95

### tert-butyl methyl(2-(methyl(7-(1-methyl-1H-pyrazol-4-yl)imidazo[1,2-c]pyrimidin-3-yl)amino)ethyl)carbamate (Compound 95)

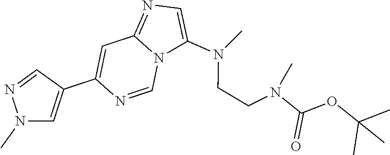 [SMILES: CN(CCN(C)c1cnc2cc(-c3cnn(C)c3)ncn12)C(=O)OC(C)(C)C]

To a solution of 6-(1-methyl-1H-pyrazol-4-yl)pyrimidin-4-amine (Compound S97) (200.0 mg, 1.14 mmol) and di-tert-butyl (((1,2-bis(1H-benzo[d][1,2,3]triazol-1-yl)ethane-1,2-diyl)bis(methylazanediyl))bis(ethane-2,1-diyl))bis(methylcarbamate) (Compound S49) (1.09 g, 1.71 mmol) in ACN (15 mL) was added ZnBr₂ (513.7 mg, 2.28 mmol) at 25° C. under nitrogen. The mixture was stirred at 80° C. for 10 h under nitrogen. The mixture was poured into IN NaOH (60 mL) and extracted with 10% MeOH in DCM (3×40 mL). The combined organic layer was washed with water (30 mL) and brine (30 mL), dried over Na₂SO₄, and filtered. The filtrate was concentrated under reduced pressure. The residue was purified by prep-TLC (EtOAc:MeOH:NH₃·H₂O=200:20:1) to give 170 mg, which was further purified by prep-HPLC to afford 31.7 mg (7%) of tert-butyl methyl(2-(methyl(7-(1-methyl-1H-pyrazol-4-yl)imidazo[1,2-c]pyrimidin-3-yl)amino)ethyl)carbamate (Compound 95) as a yellow oil. ¹HNMR (400 MHZ, CDCl₃) δ (ppm) 8.82 (s, 1H), 7.98 (s, 1H), 7.92 (s, 1H), 7.52 (s, 1H), 7.25 (s, 1H), 3.98 (s, 3H), 3.44 (br s, 2H), 3.19 (br s, 2H), 2.89 (br s, 3H), 2.86 (br s, 3H), 1.47-1.42 (m, 9H). MS (ESI) m/z [M+H]⁺ calcd. for C₁₉H₂₇N₇O₂, 386.2; Found, 386.0. HPLC purity: 220 nm: 93.8%.

Using the procedures described in Example 90, Compound 95 and reagents, starting materials, and conditions known to those skilled in the art were used to prepare the compounds shown in Table D.

TABLE D

| Compound | Name | Characterization |
|---|---|---|
| Compound 96 | tert-butyl methyl(2-(methyl(7- | ¹H NMR (400 MHz, CDCl₃) δ (ppm) |
| | (4-methyl-1H-imidazol-1- | 8.29-8.36 (m, 2H), 7.50 (s, 1H), 7.37 (br s, |
| | yl)imidazo[1,2-a]pyrimidin-3- | 1H), 6.93 (br s, 1H), 3.42 (br s, 2H), 3.14 |
| | yl)amino)ethyl)carbamate | (br s, 2H), 2.85-2.90 (m, 6H), 2.31 (d, |
| | | J = 0.8 Hz, 3H), 1.25-1.28 (m, 9H). MS |
| | | (ESI) m/z [M + H]⁺ calcd. for C₁₉H₂₇N₇O₂, |
| | | 386.2; Found, 386.1. HPLC purity: 220 |
| | | nm: 97.8%. |

### Example 91: Synthesis of Exemplary Compound 97

### tert-butyl (2-((7-(1-(2-methoxyethyl)-1H-pyrazol-4-yl)imidazo[1,2-c]pyrimidin-3-yl)(methyl)amino)ethyl)(methyl)carbamate (Compound 97)

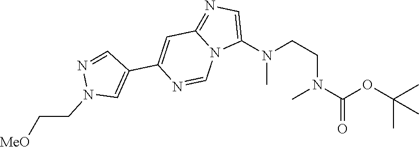 [SMILES: COCCn1cc(-c2cc3ncc(N(C)CCN(C)C(=O)OC(C)(C)C)n3cn2)cn1]

To a solution of 6-(1-(2-methoxyethyl)-1H-pyrazol-4-yl)pyrimidin-4-amine (Compound S39) (400.0 mg, 1.82 mmol) and di-tert-butyl (((1,2-bis(1H-benzo[d][1,2,3]triazol-1-yl)ethane-1,2-diyl)bis(methylazanediyl))bis(ethane-2,1-diyl))bis(methylcarbamate) (Compound S49) (1.74 g, 2.74 mmol) in ACN (20 mL) was added ZnBr₂ (821.0 mg, 3.65 mmol) at 25° C. under nitrogen. The mixture was stirred at 80° C. under nitrogen for 4 h. The mixture was poured into IN NaOH (100 mL) and extracted with 10% MeOH in DCM (3×100 mL). The combined organic layer was washed with water (100 mL) and brine (100 mL), dried over Na₂SO₄, and filtered. The filtrate was concentrated under reduced pressure. The residue was purified by prep-TLC (EtOAc:MeOH:NH₃·H₂O=200:20:1) to give the crude product (200 mg), which was purified by prep-HPLC to afford 99.0 mg (13%) tert-butyl (2-((7-(1-(2-methoxyethyl)-1H-pyrazol-4-yl)imidazo[1,2-c]pyrimidin-3-yl)(methyl)amino)ethyl)(methyl)carbamate (Compound 97) as yellow oil. ¹H NMR (400 MHZ, CDCl₃) δ (ppm) 8.80 (s, 1H), 8.01 (s, 1H), 8.00 (s, 1H), 7.51 (s, 1H), 7.22 (s, 1H), 4.35 (t, J=5.2 Hz, 2H), 3.79 (t, J=5.2 Hz, 2H), 3.50 (br s, 2H), 3.45 (s, 3H), 3.19 (br s, 2H), 2.88 (s, 3H), 2.85 (s, 3H), 1.47-1.43 (m, 9H). MS (ESI) m/z [M+H]⁺ calcd. for C₂₁H₃₁N₇O₃, 430.3; Found, 430.1. HPLC purity: 220 nm: 95.6%.

Using the procedures described in Example 91, Compound 97 and reagents, starting materials, and conditions known to those skilled in the art were used to prepare the compounds shown in Table E.

TABLE E

| Compound | Name | Characterization |
|---|---|---|
| Compound 98 | tert-butyl methyl(2-(methyl(7- | ¹H NMR (400 MHz, CDCl₃) δ (ppm) |
| | (tetrahydrofuran-3- | 8.16-8.13 (m, 1H), 7.32 (s, 1H), 6.77-6.74 |
| | yl)imidazo[1,2-a]pyrimidin-3- | (m, 1H), 4.14-4.10 (m, 1H), 4.06-4.01 |
| | yl)amino)ethyl)carbamate | (m, 1H), 3.95-3.85 (m, 2H), 3.62-3.54 |
| | | (m, 1H), 3.35 (br s, 2H), 3.07 (br s, 2H), |
| | | 2.81-2.71 (m, 6H), 2.35-2.23 (m, 2H), |
| | | 1.45-1.20 (m, 9H). MS (ESI) m/z |
| | | [M + H]⁺ calcd. for C₁₉H₂₉N₅O₃, 376.2; |
| | | Found, 376.2. HPLC purity: 220 nm: |
| | | 96.2%. |

### Example 92: Synthesis of Exemplary Compound 99

### tert-butyl methyl(2-(methyl(7-(pyridin-3-yl)imidazo[1,2-c]pyrimidin-3-yl)amino)ethyl)carbamate (Compound 99)

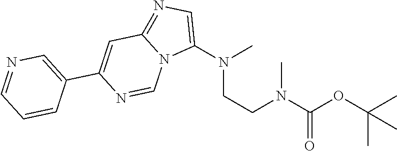 [SMILES: CN(CCN(C)c1cnc2cc(-c3cccnc3)ncn12)C(=O)OC(C)(C)C]

To a solution of 6-(pyridin-3-yl)pyrimidin-4-amine (Compound S40) (200.0 mg, 1.16 mmol) and di-tert-butyl (((1,2-bis(1H-benzo[d][1,2,3]triazol-1-yl)ethane-1,2-diyl)bis(methylazanediyl))bis(ethane-2,1-diyl))bis(methylcarbamate) (Compound S49) (1.10 g, 1.74 mmol) in ACN (20 mL) was added ZnBr₂ (392.0 mg, 1.74 mmol) at 25° C. under nitrogen. The mixture was stirred at 80° C. under nitrogen for 4 h. The mixture was poured into IN NaOH (20 mL) and extracted with DCM (3×20 mL). The combined organic layer was washed with brine (30 mL), dried over Na₂SO₄, and concentrated in vacuum to give the crude oil. The crude oil was purified by silica gel chromatography then further purified by prep-HPLC to afford 29.0 mg (7%) of tert-butyl methyl(2-(methyl(7-(pyridin-3-yl)imidazo[1,2-c]pyrimidin-3-yl)amino)ethyl)carbamate (Compound 99) as a yellow oil. ¹H NMR (400 MHZ, DMSO-d₆) δ (ppm) 9.42 (s, 1H), 9.30 (s, 1H), 8.83-8.79 (m, 1H), 8.74-8.70 (m, 1H), 8.16 (s, 1H), 7.80-7.78 (m, 1H), 7.23-7.20 (m, 1H), 3.60-3.55 (m, 2H), 3.50-3.45 (m, 2H), 2.96 (s, 3H), 2.75 (s, 3H), 1.30-1.23 (m, 9H). MS (ESI) m/z [M+H]⁺ calcd. for C₂₀H₂₆N₆O₂, 383.2; Found, 383.0. HPLC purity: 220 nm: 96.0%.

Using the procedures described in Example 92, Compound 99 and reagents, starting materials, and conditions known to those skilled in the art were used to prepare the compounds shown in Table F.

TABLE F

| Compound | Name | Characterization |
|---|---|---|
| Compound 100 | tert-butyl methyl(2-(methyl(7- | ¹H NMR (400 MHz, CDCl₃) δ (ppm) 8.81 |
| | (tetrahydrofuran-3- | (s, 1H), 7.30 (s, 1H), 7.23 (s, 1H), 4.14-4.17 |
| | yl)imidazo[1,2-c]pyrimidin-3- | (m, 1H), 4.08-4.10 (m, 1H), 3.93-3.96 |
| | yl)amino)ethyl)carbamate | (m, 1H), 3.87-3.89 (m, 1H), 3.50-3.60 |
| | | (m, 1H), 3.40-3.49 (m, 2H), 3.17 (s, |
| | | 2H), 2.87 (s, 6H), 2.35-2.40 (m, 1H), |
| | | 2.20-2.30 (m, 1H), 1.41-1.46 (m, 9H). |
| | | MS (ESI) m/z [M + H]⁺ calcd. for |
| | | C₁₉H₂₉N₅O₃, 376.2; Found, 376.0. HPLC |
| | | purity: 220 nm: 98.7%. |

### Example 93: Synthesis of Exemplary Compound 101

### tert-butyl 4-(7-(1-(2-methoxyethyl)-1H-pyrazol-4-yl)imidazo[1,2-a]pyrimidin-3-yl)piperazine-1-carboxylate (Compound 101)

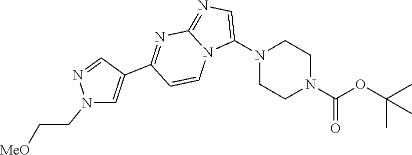 [SMILES: COCCn1cc(-c2ccn3c(N4CCN(C(=O)OC(C)(C)C)CC4)cnc3n2)cn1]

To a solution of 4-(1-(2-methoxyethyl)-1H-pyrazol-4-yl)pyrimidin-2-amine (Compound S43) (200 mg, 0.91 mmol) and di-tert-butyl 4,4′-(1,2-bis(1H-benzo[d][1,2,3]triazol-1-yl)ethane-1,2-diyl)bis(piperazine-1-carboxylate) (Compound S47) (577 mg, 0.91 mmol) in DCE (25 mL) was added ZnBr₂ (205 mg, 0.91 mmol) at 25° C. under nitrogen. The mixture was stirred at 80° C. for 4 h under nitrogen. The solution was filtered, and the filtrate was collected. The mixture was poured into IN NaOH (20 mL) and extracted with 10% MeOH in DCM (3×20 mL). The combined organic layer was washed with water (20 mL) and brine (20 mL), dried over Na₂SO₄, and filtered. The filtrate was concentrated under reduced pressure. The residue was purified by prep-TLC (EtOAc:MeOH:NH₃·H₂O=200:20:1) to afford 77 mg (19%) of tert-butyl 4-(7-(1-(2-methoxyethyl)-1H-pyrazol-4-yl)imidazo[1,2-a]pyrimidin-3-yl)piperazine-1-carboxylate (Compound 101) as a yellow solid. ¹H NMR (400 MHz, CDCl₃) δ (ppm) 8.22 (s, 1H), 8.20 (d, J=7.2 Hz, 1H), 8.08 (s, 1H), 7.34 (s, 1H), 7.04 (d, J=7.2 Hz, 1H), 4.36 (t, J=5.2 Hz, 2H), 3.79 (t, J=4.8 Hz, 2H), 3.66-3.60 (m, 4H), 3.36 (s, 3H), 3.03-2.98 (m, 4H), 1.50 (s, 9H). MS (ESI) m/z [M+H]⁺ calcd. for C₂₁H₂₉N₇O₃, 428.2; Found, 428.1. HPLC purity: 220 nm: 98.8%.

Using the procedures described in Example 93, Compound 101 and reagents, starting materials, and conditions known to those skilled in the art were used to prepare the compounds shown in Table G.

TABLE G

| Compound | Name | Characterization |
|---|---|---|
| Compound | tert-butyl 4-(7-(pyridin-3-yl)imidazo[1,2- | ¹H NMR (400 MHz, CDCl₃) δ |
| 102 | a]pyrimidin-3-yl)piperazine-1-carboxylate | (ppm) 9.31 (s, 1H), 8.72 (d, J = |
| | | 5.2 Hz, 1H), 8.58-8.60 (m, |
| | | 1H), 8.37 (d, J = 7.2 Hz, 1H), |
| | | 7.49-7.44 (m 2H), 7.41-7.39 |
| | | (m, 1H), 3.68-3.64 (m, 4H), |
| | | 3.08-3.03 (m, 4H), 1.51 (s, |
| | | 9H). MS (ESI) m/z [M + H]⁺ |
| | | calcd. for C₂₀H₂₄N₆O₂, 381.2; |
| | | Found, 381.1. HPLC purity: |
| | | 220 nm: 98.2%. |
| Compound | tert-butyl 4-(7-(tetrahydrofuran-3- | ¹H NMR (400 MHz, CDCl₃) δ |
| 103 | yl)imidazo[1,2-a]pyrimidin-3- | (ppm) 8.20 (d, J = 7.2 Hz, 1H), |
| | yl)piperazine-1-carboxylate | 7.37 (s, 1H), 6.82 (d, J = 7.2 |
| | | Hz, 1H), 4.18-4.16 (m, 1H), |
| | | 4.12-4.07 (m, 1H), 4.01-3.91 |
| | | (m, 2H), 3.68-3.61 (m, |
| | | 5H), 3.00-2.97 (m, 4H), |
| | | 2.39-2.31 (m, 2H), 1.50 (s, 9H). |
| | | MS (ESI) m/z [M + H]⁺ calcd. |
| | | for C₁₉H₂₇N₅O₃, 374.2; |
| | | Found, 374.1. HPLC purity: |
| | | 220 nm: 98.5%. |
| Compound | tert-butyl 4-(7-(4-methyl-1H-imidazol-1- | ¹H NMR (400 MHz, CDCl₃) δ |
| 104 | yl)imidazo[1,2-a]pyrimidin-3- | (ppm) 8.37-8.35 (m, 2H), |
| | yl)piperazine-1-carboxylate | 7.54 (s, 1H), 7.39 (s, 1H), 6.99 |
| | | (d, J = 7.2 Hz, 1H), 3.67-3.64 |
| | | (m, 4H), 3.05-3.02 (m, 4H), |
| | | 2.33 (s, 3H), 1.52 (s, 9H). MS |
| | | (ESI) m/z [M + H]⁺ calcd. for |
| | | C₁₉H₂₅N₇O₂, 384.2; Found, |
| | | 384.0. HPLC purity: 220 nm: |
| | | 99.0%. |
| Compound | tert-butyl 4-(7-(1-(2-methoxyethyl)-1H- | ¹H NMR (400 MHz, CDCl₃) δ |
| 105 | pyrazol-4-yl)imidazo[1,2-a]pyrimidin-3- | (ppm) 8.20-8.30 (m, 2H), |
| | yl)-1,4-diazepane-1-carboxylate | 8.08 (s, 1H), 7.36 (s, 1H), |
| | | 7.11-7.12 (m, 1H), 4.35-4.37 (m, |
| | | 2H), 3.72-3.79 (m, 2H), |
| | | 3.50-3.57 (m, 4H), 3.58 (s, 3H), |
| | | 3.18-3.25 (m, 4H), 1.90-1.97 |
| | | (m, 2H), 1.53-1.48 (m, |
| | | 9H). MS (ESI) m/z [M + H]⁺ |
| | | calcd. for C₂₂H₃₁N₇O₃, 442.3; |
| | | Found, 442.1. HPLC purity: |
| | | 220 nm: 97.7%. |
| Compound | tert-butyl 4-(7-(pyridin-3-yl)imidazo[1,2- | ¹H NMR (400 MHz, CDCl₃) δ |
| 106 | alpyrimidin-3-yl)-1,4-diazepane-1- | (ppm) 9.31 (s, 1H), 8.74-8.72 |
| | carboxylate | (m, 1H), 8.53-8.55 (m, |
| | | 1H), 8.36-8.43 (m, 1H), |
| | | 7.42-7.50 (m, 3H), 3.57-3.66 (m, |
| | | 4H), 3.21-3.30 (m, 4H), |
| | | 1.92-2.02 (m, 2H), 1.42-1.58 (m, |
| | | 9H). MS (ESI) m/z [M + H]⁺ |
| | | calcd. for C₂₁H₂₆N₆O₂, 395.2; |
| | | Found, 395.1. HPLC purity: |
| | | 220 nm: 97.7%. |
| Compound | tert-butyl 4-(7-(tetrahydrofuran-3- | ¹H NMR (400 MHz, CDCl₃) δ |
| 107 | yl)imidazo[1,2-a]pyrimidin-3-yl)-1,4- | (ppm) 8.22-8.17 (m, 1H), |
| | diazepane-1-carboxylate | 7.37 (d, J = 5.6 Hz, 1H), 6.80 |
| | | (d, J = 6.8 Hz, 1H), 4.20-4.16 |
| | | (m, 1H), 4.10-4.07 (m, 1H), |
| | | 4.01-3.91 (m, 2H), 3.66-3.55 |
| | | (m, 5H), 3.26-3.21 (m, , |
| | | 3H), 3.17-3.15 (m, 1H), 2.40-2.35 |
| | | (m, 2H), 1.94-1.87 (m, |
| | | 2H), 1.52-1.48 (m, 9H). MS |
| | | (ESI) m/z [M + H]⁺ calcd. for |
| | | C₂₀H₂₉N₅O₃, 388.2; Found, |
| | | 388.1. HPLC purity: 220 nm: |
| | | 99.3%. |
| Compound | tert-butyl methyl(2-(methyl(7-(1-methyl- | ¹H NMR (400 MHz, CDCl₃) δ |
| 108 | 1H-pyrazol-4-yl)imidazo[1,2-a]pyrimidin- | (ppm) 8.14-8.26 (m, 2H), |
| | 3-yl)amino)ethyl)carbamate | 8.03 (s, 1H), 7.37 (s, 1H), 7.07 |
| | | (d, J = 7.2 Hz, 1H), 3.99 (s, |
| | | 3H), 3.42 (br s, 2H), 3.13 (br |
| | | s, 2H), 2.89 (br s, 3H), 2.85 (s, |
| | | 3H), 1.42-1.33 (m, 9H). MS |
| | | (ESI) m/z [M + H]⁺ calcd. for |
| | | C₁₉H₂₇N₇O₂, 386.2; Found, |
| | | 386.1. HPLC purity: 220 nm: |
| | | 97.1%. |
| Compound | tert-butyl (2-((7-(1-(2-methoxyethyl)-1H- | ¹H NMR (400 MHz, CDCl₃) δ |
| 109 | pyrazol-4-yl)imidazo[1,2-a]pyrimidin-3- | (ppm) 8.23 (s, 1H), 8.20 (br s, |
| | yl)(methyl)amino)ethyl)(methyl)carbamate | 1H), 8.09 (s, 1H), 7.37 (s, 1H), |
| | | 7.06 (d, J = 6.8 Hz, 1H), 4.37 |
| | | (t, J = 5.2 Hz, 2H), 3.81 (t, J = |
| | | 5.2 Hz, 2H), 3.45 (br s, 2H), |
| | | 3.37 (s, 3H), 3.14 (br s, 2H), |
| | | 2.90 (br s, 3H), 2.86 (s, 3H), |
| | | 1.47-1.43 (m, 9H). MS (ESI) |
| | | m/z [M + H]⁺ calcd. for |
| | | C₂₁H₃₁N₇O₃, 430.3; Found, |
| | | 430.1. HPLC purity: 220 nm: |
| | | 98.8%. |
| Compound | tert-butyl 1-(7-(1-methyl-1H-pyrazol-4- | ¹H NMR (400 MHz, CDCl₃) δ |
| 110 | yl)imidazo[1,2-a]pyrimidin-3- | (ppm) 8.25 (d, J = 6.8 Hz, 1H), |
| | yl)pyrrolidine-3-carboxylate | 8.11 (s, 1H), 8.03 (s, 1H), 7.29 |
| | | (s, 1H), 7.01 (d, J = 7.2 Hz, |
| | | 1H), 3.98 (s, 3H), 3.50-3.52 |
| | | (m, 1H), 3.22-3.26 (m, 3H), |
| | | 3.12-3.15 (m, 1H), 2.25~2.31 |
| | | (m, 2H), 1.49 (s, 9H). MS |
| | | (ESI) m/z [M + H]⁺ calcd. for |
| | | C₁₉H₂₄N₆O₂, 369.2; Found, |
| | | 369.1. HPLC purity: 220 nm: |
| | | 98.9%. |

### Example 94: Synthesis of Exemplary Compound 111

### tert-butyl 4-(7-(4-methyl-1H-imidazol-1-yl)imidazo[1,2-a]pyrimidin-3-yl)-1,4-diazepane-1-carboxylate (Compound 111)

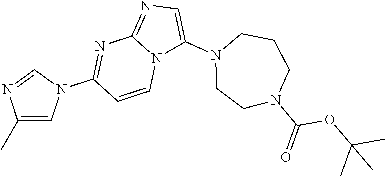 [SMILES: Cc1cn(-c2ccn3c(N4CCCN(C(=O)OC(C)(C)C)CC4)cnc3n2)cn1]

To a solution of 4-(4-methyl-1H-imidazol-1-yl)pyrimidin-2-amine (Compound S46) (0.40 g, 2.30 mmol) and di-tert-butyl 4,4′-(1,2-bis(1H-benzo[d][1,2,3]triazol-1-yl)ethane-1,2-diyl)bis(1,4-diazepane-1-carboxylate) (Compound S48) (2.28 g, 3.45 mmol) in ACN (30 mL) was added ZnBr₂ (1.55 g, 6.90 mmol) at 25° C. under nitrogen. The mixture was stirred at 80° C. for 6 h. The mixture was poured into IN NaOH (20 mL) and extracted with DCM (50 mL×3). The combined organic layers were washed with brine (40 mL) and dried over Na₂SO₄, concentrated in vacuum to give the crude oil. The crude oil was purified by silica gel chromatography then further purified by prep-HPLC to afford 20.0 mg (2%) of tert-butyl 4-(7-(4-methyl-1H-imidazol-1-yl)imidazo[1,2-a]pyrimidin-3-yl)-1,4-diazepane-1-carboxylate (Compound 111) as a yellow solid. ¹H NMR (400 MHZ, CDCl₃) δ (ppm) 8.41-8.48 (m, 1H), 8.32-8.39 (m, 1H), 7.54 (s, 1H), 7.36-7.43 (m, 1H), 7.02-7.04 (m, 1H), 3.56-3.64 (m, 4H), 3.19-3.30 (m, 4H), 2.32 (s, 3H), 1.90-1.97 (m, 2H), 1.53-1.49 (m, 9H). MS (ESI) m/z [M+H]⁺ calcd. for C₂₀H₂₇N₇O₂, 398.2; Found, 398.0. HPLC purity: 220 nm: 95.7%.

Using the procedures described in Example 94, Compound 111 and reagents, starting materials, and conditions known to those skilled in the art were used to prepare the compounds shown in Table H.

TABLE H

| Compound | Name | Characterization |
|---|---|---|
| Compound | tert-butyl methyl(2-(methyl(7-(pyridin-3- | ¹H NMR (400 MHz, CDCl₃) δ |
| 112 | yl)imidazo[1,2-a]pyrimidin-3- | (ppm) 9.31 (d, J = 1.6 Hz, 1H), |
| | yl)amino)ethyl)carbamate | 8.70 (d, J = 4.4 Hz, 1H), 8.56-8.60 |
| | | (m, 1H), 8.33-8.42 (m, |
| | | 1H), 7.48-7.51 (m, 1H), |
| | | 7.44-7.46 (m, 1H), 7.38-7.40 (m, |
| | | 1H), 3.44 (br s, 2H), 3.17 (br |
| | | s, 2H), 2.89 (s, 3H), 2.84 (br s, |
| | | 3H), 1.47-1.42 (m, 9H). MS |
| | | (ESI) m/z [M + H]⁺ calcd. for |
| | | C₂₀H₂₆N₆O₂, 383.2; Found, |
| | | 383.0. HPLC purity: 220 nm: |
| | | 96.0%. |

### Example 95: Synthesis of Exemplary Compound 113

### 3-(4-(5-benzylpyrimidin-2-yl)piperazin-1-yl)-7-(1-methyl-1H-pyrazol-4-yl)imidazo[1,2-c]pyrimidine (Compound 113)

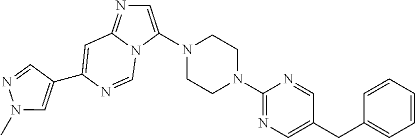 [SMILES: Cn1cc(-c2cc3ncc(N4CCN(c5ncc(Cc6ccccc6)cn5)CC4)n3cn2)cn1]

To a mixture of 7-(1-methyl-1H-pyrazol-4-yl)-3-(piperazin-1-yl)imidazo[1,2-c]pyrimidine hydrochloride salt (Compound S52) (250.0 mg, 0.78 mmol) in dioxane (8.0 mL) was added 5-benzyl-2-chloropyrimidine (Compound S88) (240.0 mg, 1.17 mmol), Pd₂(dba)₃ (143.2 mg, 0.16 mmol), BINAP (48.7 mg, 0.08 mmol) and Cs₂CO₃ (764.2 mg, 2.35 mmol) at 25° C. under nitrogen. The mixture was stirred at 80° C. for 16 h under nitrogen. The mixture was poured into H₂O (20 mL) and extracted with EtOAc (3×10 mL). The combined organic layers were washed with H₂O (10×3 mL) and brine (10 mL) and dried over Na₂SO₄, concentrated in vacuum to give the crude oil, which was purified by prep-HPLC to afford 2.4 mg (1%) of 3-(4-(5-benzylpyrimidin-2-yl)piperazin-1-yl)-7-(1-methyl-1H-pyrazol-4-yl)imidazo[1,2-c]pyrimidine (Compound 113) as an off-white solid. ¹HNMR (400 MHZ, DMSO-d₆) δ 9.13 (s, 1H), 8.31 (s, 2H), 8.24 (s, 1H), 8.00 (s, 1H), 7.57 (s, 1H), 7.27-7.29 (m, 2H), 7.22-7.24 (m, 4H), 3.88 (s, 3H), 3.83-3.86 (m, 4H), 3.80 (s, 2H), 3.32-3.25 (m, 4H). MS (ESI) m/z [M+H]⁺ calcd. for C₂₅H₂₅N₉, 452.2; Found, 452.1. HPLC purity: 220 nm: 96.1%.

Using the procedures described in Example 95, Compound 113 and reagents, starting materials, and conditions known to those skilled in the art were used to prepare the compounds shown in Table I.

TABLE I

| Compound | Name | Characterization |
|---|---|---|
| Compound | 3-(4-(5-benzylpyrimidin-2-yl)-1,4- | ¹H NMR (400 MHz, DMSO- |
| 114 | diazepan-1-yl)-7-(1-methyl-1H-pyrazol-4- | d₆) δ (ppm) 9.02 (s, 1H), 8.22 |
| | yl)imidazo[1,2-c]pyrimidine | (s, 2H), 8.20 (s, 1H), 7.97 (s, |
| | | 1H), 7.51 (s, 1H), 7.24-7.26 |
| | | (m, 2H), 7.16-7.20 (m, 3H), |
| | | 7.05 (s, 1H), 3.90-3.92 (m, |
| | | 2H), 3.87 (s, 3H), 3.73 (s, 2H), |
| | | 3.65-3.67 (m, 2H), 3.60-3.63 |
| | | (m, 2H), 3.49-3.50 (m, 2H), |
| | | 1.90-1.93 (m, 2H). MS (ESI) |
| | | m/z [M + H]⁺ calcd. for |
| | | C₂₆H₂₇N₉, 466.2; Found, |
| | | 466.1. HPLC purity: 220 nm: |
| | | 92.2%. |

### Example 96: Synthesis of Exemplary Compound 115

### (R)-1-phenylethyl 4-(7-(1-methyl-1H-pyrazol-4-yl)imidazo[1,2-c]pyrimidin-3-yl)piperazine-1-carboxylate (Compound 115)

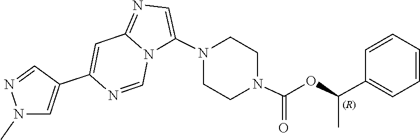

To a mixture of 7-(1-methyl-1H-pyrazol-4-yl)-3-(piperazin-1-yl)imidazo[1,2-c]pyrimidine hydrochloride salt (120.0 mg, 0.38 mmol, Compound S52) in ACN (4 mL) was added DIPEA (145.5 mg, 1.13 mmol) and the mixture was stirred at 25° C. for 10 min then di-tert-butyl 1,1′-(1,2-bis(1H-benzo[d][1,2,3]triazol-1-yl)ethane-1,2-diyl)bis(pyrrolidine-3-carboxylate) (Compound S50) (107.8 mg, 0.38 mmol) was added at 25° C. The mixture was stirred at 80° C. for 2 h. The mixture was cooled to 25° C., and concentrated to give the crude oil, which was purified by prep-HPLC to afford 23.0 mg (14%) of (R)-1-phenylethyl 4-(7-(1-methyl-1H-pyrazol-4-yl)imidazo[1,2-c]pyrimidin-3-yl)piperazine-1-carboxylate (Compound 115) as a yellow solid. ¹H NMR (400 MHZ, CDCl₃) δ 8.76 (s, 1H), 7.92 (s, 1H), 7.86 (s, 1H), 7.36-7.37 (m, 4H), 7.27-7.28 (m, 2H), 6.80 (s, 1H), 5.86 (q, J=6.4 Hz, 1H), 3.96 (s, 1H), 3.69-3.65 (m, 4H), 3.33-3.25 (m, 4H), 1.56 (d, J=6.4 Hz, 3H). MS (ESI) m/z [M+H]⁺ calcd. for C₂₃H₂₅N₇O₂, 432.2; Found, 432.0. HPLC purity: 220 nm: 95.1%.

Using the procedures described in Example 96, Compound 115 and reagents, starting materials, and conditions known to those skilled in the art were used to prepare the compounds shown in Table J.

TABLE J

| Compound | Name | Characterization |
|---|---|---|
| Compound | (R)-1-phenylethyl 4-(7-(1-methyl-1H- | ¹H NMR (400 MHz, CDCl₃) δ |
| 116 | pyrazol-4-yl)imidazo[1,2-a]pyrimidin-3- | (ppm) 8.25-8.27 (m, 2H), |
| | yl)piperazine-1-carboxylate | 8.05 (s, 1H), 7.37-7.40 (s, |
| | | 1H), 7.36-7.37 (m, 4H), |
| | | 7.28-7.35 (m, 1H), 5.86 (q, J = 6.4 |
| | | Hz, 1H), 4.00 (s, 3H), 3.75-3.71 |
| | | (m, 4H), 3.05-3.01 (m, |
| | | 4H), 1.59 (d, J = 6.8 Hz, 3H). |
| | | MS (ESI) m/z [M + H]⁺ calcd. |
| | | for C₂₃H₂₅N₇O₂, 432.2; |
| | | Found, 432.0. HPLC purity: |
| | | 220 nm: 97.0%. |
| Compound | (R)-1-phenylethyl 4-(7-(1-methyl-1H- | ¹H NMR (400 MHz, CDCl₃) δ |
| 117 | pyrazol-4-yl)imidazo[1,2-a]pyrimidin-3- | (ppm) 8.12 (s, 1H), 8.07-8.10 |
| | yl)-1,4-diazepane-1-carboxylate | (m, 1H), 8.02 (s, 1H), |
| | | 7.25-7.40 (m, 6H), 6.92-6.95 (m, |
| | | 1H), 5.86-5.90 (m, 1H), 3.98 |
| | | (s, 3H), 3.55-3.70 (m, 4H), |
| | | 3.16-3.28 (m, 4H), 1.93-1.96 |
| | | (m, 2H), 1.56-1.60 (m, 3H). |
| | | MS (ESI) m/z [M + H]⁺ calcd. |
| | | for C₂₄H₂₇N₇O₂, 446.2; |
| | | Found, 446.1. HPLC purity: |
| | | 220 nm: 98.1%. |

### Example 97: Synthesis of Exemplary Compound 118

### 4-(7-(1-methyl-1H-pyrazol-4-yl)imidazo[1,2-c]pyrimidin-3-yl)-N-(4-(trifluoromethyl)benzyl)piperazine-1-carboxamide (Compound 118)

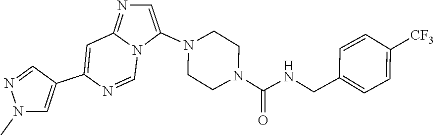 [SMILES: Cn1cc(-c2cc3ncc(N4CCN(C(=O)NCc5ccc(C(F)(F)F)cc5)CC4)n3cn2)cn1]

To a mixture of 7-(1-methyl-1H-pyrazol-4-yl)-3-(piperazin-1-yl)imidazo[1,2-c]pyrimidine hydrochloride salt (150.0 mg, 0.47 mmol) in ACN (8 mL) was added DIPEA (181.9 mg, 1.41 mmol) and the mixture was stirred at 25° C. for 10 min. 1-(isocyanatomethyl)-4-(trifluoromethyl)benzene (94.4 mg, 0.47 mmol) was added at 25° C. The mixture was stirred at 80° C. for 2 h. The mixture was cooled to 25° C., and concentrated in vacuum to give the crude oil. The crude oil was purified by prep-HPLC to afford 22.1 mg (10%) of 4-(7-(1-methyl-1H-pyrazol-4-yl)imidazo[1,2-c]pyrimidin-3-yl)-N-(4-(trifluoromethyl)benzyl)piperazine-1-carboxamide (Compound 118) as a yellow solid. ¹H NMR (400 MHZ, CDCl₃) δ (ppm) 8.78 (s, 1H), 7.93 (s, 1H), 7.88 (s, 1H), 7.60-7.70 (m, 2H), 7.35-7.45 (m, 2H), 7.30 (s, 1H), 6.82 (s, 1H), 4.95 (br s, 1H), 4.52 (d, J=5.2 Hz, 2H), 3.96 (s, 3H), 3.58-3.60 (m, 4H), 3.39-3.41 (m, 4H). MS (ESI) m/z [M+H]⁺ calcd. for C₂₃H₂₃F₃N₈O, 485.2; Found, 485.0. HPLC purity: 220 nm: 95.6%.

Using the procedures described in Example 97, Compound 118 and reagents, starting materials, and conditions known to those skilled in the art were used to prepare the compounds shown in Table K.

TABLE K

| Compound | Name | Characterization |
|---|---|---|
| Compound | 4-(7-(1-methyl-1H-pyrazol-4- | ¹H NMR (400 MHz, CDCl₃) δ |
| 119 | yl)imidazo[1,2-a]pyrimidin-3-yl)-N-(4- | (ppm) 8.24 (d, J = 7.2 Hz, 1H), |
| | (trifluoromethyl)benzyl)piperazine-1- | 8.15 (s, 1H), 8.04 (s, 1H), 7.59 |
| | carboxamide | (d, J = 8.0 Hz, 2H), 7.45 (d, |
| | | J = 8.0 Hz, 2H), 7.36 (s, 1H), |
| | | 7.13-7.11 (m, 1H), 5.18 (br s, |
| | | 1H), 4.53 (d, J = 5.2 Hz, 2H), |
| | | 3.99 (s, 3H), 3.65-3.63 (m, |
| | | 4H), 3.08-3.04 (m, 4H). MS |
| | | (ESI) m/z [M + H]⁺ calcd. for |
| | | C₂₃H₂₃F₃N₈O, 485.2; Found, |
| | | 485.1. HPLC purity: 220 nm: |
| | | 98.9%. |
| Compound | 4-(7-(1-methyl-1H-pyrazol-4- | ¹H NMR (400 MHz, CDCl₃) δ |
| 120 | yl)imidazo[1,2-a]pyrimidin-3-yl)-N-(4- | (ppm) 8.22 (d, J = 7.2 Hz, 1H), |
| | (trifluoromethyl)benzyl)-1,4-diazepane-1- | 8.13 (s, 1H), 8.02 (s, 1H), 7.57 |
| | carboxamide | (d, J = 8.0 Hz, 2H), 7.45 (d, |
| | | J = 8.0 Hz, 2H), 7.35 (s, 1H), |
| | | 7.04 (d, J = 7.2 Hz, 1H), |
| | | 5.10-5.06 (m, 1H), 4.54 (d, J = 5.6 |
| | | Hz, 1H), 3.98 (s, 3H), 3.63-3.72 |
| | | (m, 2H), 3.55-3.58 (m, |
| | | 2H), 3.31-3.34 (m, 2H), |
| | | 3.24-3.27 (m, 2H), 2.00-2.03 (m, |
| | | 2H). MS (ESI) m/z [M + H]⁺ |
| | | calcd. for C₂₄H₂₅F₃N₈O, |
| | | 499.2; Found, 499.1. HPLC |
| | | purity: 220 nm: 95.2%. |

### Example 98: Synthesis of Exemplary Compound 121

### 3-(4-(5-benzylpyrimidin-2-yl)piperazin-1-yl)-7-(1-methyl-1H-pyrazol-4-yl)imidazo[1,2-a]pyrimidine (Compound 121)

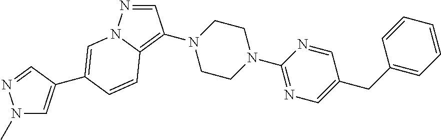 [SMILES: Cn1cc(-c2ccc3c(N4CCN(c5ncc(Cc6ccccc6)cn5)CC4)cnn3c2)cn1]

To a solution of 7-(1-methyl-1H-pyrazol-4-yl)-3-(piperazin-1-yl)imidazo[1,2-a]pyrimidine hydrochloride salt (Compound S54) (0.15 g, 0.50 mmol) in DMF (10 mL) was added 5-benzyl-2-chloropyrimidine (Compound S88) (0.1 g, 0.50 mmol) and K₂CO₃ (0.21 g, 1.50 mmol) at 25° C. under nitrogen. The mixture was stirred at 80° C. for 4 h under nitrogen. The mixture was poured into H₂O (20 mL) and extracted with EtOAc (4×30 mL). The combined organic layer was washed with H₂O (10×3 mL) and brine (10 mL), dried over Na₂SO₄, and concentrated in vacuum to give the crude oil. The crude oil was purified by prep-HPLC to afford 22 mg (10%) 3-(4-(5-benzylpyrimidin-2-yl)piperazin-1-yl)-7-(1-methyl-1H-pyrazol-4-yl)imidazo[1,2-a]pyrimidine (Compound 121) as a yellow solid. ¹H NMR (400 MHz, CDCl₃) δ (ppm) 8.26 (d, J=7.2 Hz, 1H), 8.22 (s, 2H), 8.13 (s, 1H), 8.04 (s, 1H), 7.35 (s, 1H), 7.28-7.31 (m, 2H), 7.24-7.26 (m, 1H), 7.17-7.22 (m, 2H), 7.03-7.05 (m, 1H), 4.38-4.01 (m, 4H), 3.99 (s, 3H), 3.82 (s, 2H), 3.08-3.11 (m, 4H). MS (ESI) m/z [M+H]⁺ calcd. for C₂₅H₂₅N₉, 452.2; Found, 452.1. HPLC purity: 220 nm: 99.6%.

Using the procedures described in Example 98, Compound 121 and reagents, starting materials, and conditions known to those skilled in the art were used to prepare the compounds shown in Table L.

TABLE L

| Compound | Name | Characterization |
|---|---|---|
| Compound | 3-(4-(5-benzylpyrimidin-2-yl)-1,4- | ¹H NMR (400 MHz, CDCl₃) δ |
| 122 | diazepan-1-yl)-7-(1-methyl-1H-pyrazol-4- | (ppm) 8.21 (s, 2H), 8.13 (s, |
| | yl)imidazo[1,2-a]pyrimidine | 1H), 8.09 (d, J = 7.2 Hz, 1H), |
| | | 8.00 (s, 1H), 7.26-7.28 (m, |
| | | 3H), 7.23-7.24 (m, 3H), 6.97 |
| | | (d, J = 7.2 Hz, 1H), 3.95-4.01 |
| | | (m, 7H), 3.86 (s, 2H), |
| | | 3.33-3.35 (m, 2H), 3.20-3.22 (m, |
| | | 2H), 2.07-2.02 (m, 1H). MS |
| | | (ESI) m/z [M + H]⁺ calcd. for |
| | | C₂₆H₂₇N₉, 466.2; Found, |
| | | 466.1. HPLC purity: 220 nm: |
| | | 99.6%. |

### Example 99: Synthesis of Exemplary Compound 123

### N¹-(5-benzylpyrimidin-2-yl)-N²-(6-(1-methyl-1H-pyrazol-4-yl)pyrazolo[1,5-a]pyridin-3-yl)ethane-1,2-diamine (Compound 123)

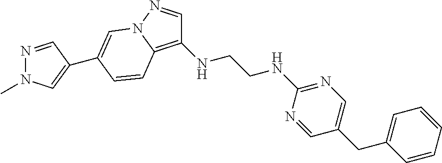 [SMILES: Cn1cc(-c2ccc3c(NCCNc4ncc(Cc5ccccc5)cn4)cnn3c2)cn1]

Step 1. Preparation of tert-butyl (2-((6-(1-methyl-1H-pyrazol-4-yl)pyrazolo[1,5-a]pyridin-3-yl)amino)ethyl)carbamate

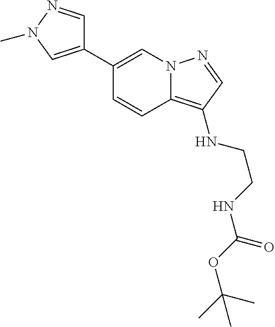 [SMILES: Cn1cc(-c2ccc3c(NCCNC(=O)OC(C)(C)C)cnn3c2)cn1]

To a solution of 6-(1-methyl-1H-pyrazol-4-yl)pyrazolo[1,5-a]pyridin-3-amine (Compound S56) (0.80 g, 3.75 mmol) dissolved in DMF (20 mL) was added tert-butyl (2-bromoethyl)carbamate (1.26 g, 5.63 mmol) and K₂CO₃ (1.56 g, 11.26 mmol). The resulting solution was stirred for 16 h at 90° C. under nitrogen atmosphere. The mixture was allowed to cool to rt. The resulting mixture was diluted with water (100 mL) and extracted with EtOAc (3×100 mL). The combined organic layers were washed with brine (3×100 mL) and dried over anhydrous Na₂SO₄. After filtration, the filtrate was concentrated under reduced pressure. The crude product was purified by Prep-HPLC to afford 220 mg (11%) of tert-butyl (2-((6-(1-methyl-1H-pyrazol-4-yl)pyrazolo[1,5-a]pyridin-3-yl)amino)ethyl)carbamate as a light yellow solid. ¹H NMR (400 MHZ, DMSO-d₆) δ (ppm) 8.68 (s, 1H), 8.17 (s, 1H), 7.93 (s, 1H), 7.62-7.50 (m, 2H), 7.15 (d, J=9.3 Hz, 1H), 6.93-6.79 (m, 1H), 4.84-4.70 (m, 1H), 3.87 (s, 3H), 3.18-3.00 (m, 4H), 1.39 (s, 9H). MS (ESI) m/z [M+H]⁺ calcd. for C₁₈H₂₄N₆O₂, 357.2; found, 357.2.

### Step 2. Preparation of N¹-(6-(1-methyl-1H-pyrazol-4-yl)pyrazolo[1,5-a]pyridin-3-yl)ethane-1,2-diamine

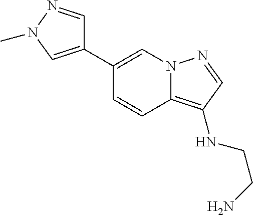 [SMILES: Cn1cc(-c2ccc3c(NCCN)cnn3c2)cn1]

To a solution of tert-butyl (2-((6-(1-methyl-1H-pyrazol-4-yl)pyrazolo[1,5-a]pyridin-3-yl)amino)ethyl)carbamate (200 mg, 0.56 mmol, as prepared in the previous step) dissolved in DCM (6 mL) was added 4M HCl in dioxane (2 mL). The resulting solution was stirred for 1 h at 0° C. under nitrogen atmosphere. The resulting mixture was concentrated under reduced pressure to afford 120 mg (83%) of N¹-(6-(1-methyl-1H-pyrazol-4-yl)pyrazolo[1,5-a]pyridin-3-yl)ethane-1,2-diamine as a yellow solid. MS (ESI) m/z [M+H]⁺ calcd. for C₁₃H₁₆N₆, 257.1; found, 257.1.

### Step 3. Preparation of N¹-(5-benzylpyrimidin-2-yl)-N²-(6-(1-methyl-1H-pyrazol-4-yl)pyrazolo[1,5-a]pyridin-3-yl)ethane-1,2-diamine (Compound 123)

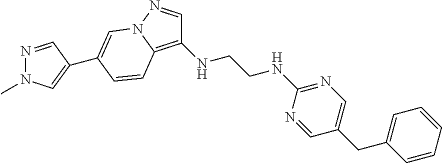 [SMILES: Cn1cc(-c2ccc3c(NCCNc4ncc(Cc5ccccc5)cn4)cnn3c2)cn1]

To a solution of N¹-(6-(1-methyl-1H-pyrazol-4-yl)pyrazolo[1,5-a]pyridin-3-yl)ethane-1,2-diamine (100 mg, 0.39 mmol, as prepared in the previous step) dissolved in IPA (5 mL) was added 5-benzyl-2-chloropyrimidine (Compound S88) (87.83 mg, 0.43 mmol) and DIPEA (151.3 mg, 1.17 mmol). The resulting solution was stirred for 16 h at 120° C. under nitrogen. The mixture was allowed to cool to rt, diluted with water (10 mL), and extracted with EtOAc (3×20 mL). The combined organic layers were washed with brine (3×10 mL) and dried over anhydrous Na₂SO₄. After filtration, the filtrate was concentrated under reduced pressure. The crude product was purified by Prep-HPLC to afford 10.4 mg (6%) of N¹-(5-benzylpyrimidin-2-yl)-N²-(6-(1-methyl-1H-pyrazol-4-yl)pyrazolo[1,5-a]pyridin-3-yl)ethane-1,2-diamine (Compound 123) as a yellow solid. ¹H NMR (400 MHz, DMSO-d₆) δ (ppm) 8.67 (s, 1H), 8.20 (s, 2H), 8.17 (s, 1H), 7.93 (s, 1H), 7.60-7.55 (m, 2H), 7.33-7.16 (m, 5H), 7.17-7.10 (m, 1H), 7.07 (t, J=6.0 Hz, 1H), 4.89 (t, J=6.3 Hz, 1H), 3.86 (s, 3H), 3.75 (s, 2H), 3.48-3.43 (m, 2H), 3.24-3.15 (m, 2H). MS (ESI) m/z [M+H]⁺ calcd. for C₂₄H₂₄N₈, 425.2; found, 425.2; HPLC purity: 254 nm: 99.5%.

Using the procedures described in Example 99, Compound 123 and reagents, starting materials, and conditions known to those skilled in the art were used to prepare the compounds shown in Table M.

TABLE M

| Compound | Name | Characterization |
|---|---|---|
| Compound | N¹-(5-benzylpyrimidin-2-yl)-N³-(6-(1- | ¹H NMR (400 MHz, DMSO- |
| 124 | methyl-1H-pyrazol-4-yl)pyrazolo[1,5- | d₆) δ (ppm) 8.65 (s, 1H), 8.17 |
| | a]pyridin-3-yl)propane-1,3-diamine | (s, 3H), 7.93 (s, 1H), 7.59 (d, |
| | | J = 9.2 Hz, 1H), 7.49 (s, 1H), |
| | | 7.33-7.05 (m, 7H), 4.72 (t, |
| | | J = 6.4 Hz, 1H), 3.86 (s, 3H), |
| | | 3.74 (s, 2H), 341-3.35 (m, |
| | | 2H), 3.13-3.09 (m, 2H), 1.86-1.75 |
| | | (m, 2H). MS (ESI) m/z |
| | | [M + H]⁺ calcd. for C₂₅H₂₆N₈, |
| | | 439.23; found, 439.15; HPLC |
| | | purity: 254 nm: 95.3%. |

### Example 100: Synthesis of Exemplary Compound 125

### N¹-(5-benzylpyrimidin-2-yl)-N²-methyl-N²-(6-(1-methyl-1H-pyrazol-4-yl)pyrazolo[1,5-a]pyridin-3-yl)ethane-1,2-diamine (Compound 125)

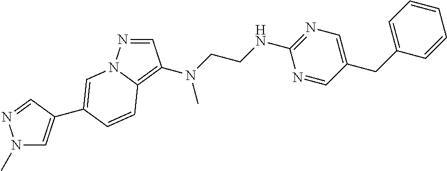 [SMILES: CN(CCNc1ncc(Cc2ccccc2)cn1)c1cnn2cc(-c3cnn(C)c3)ccc12]

### Step 1. Preparation of N¹-methyl-N¹-(6-(1-methyl-1H-pyrazol-4-yl)pyrazolo[1,5-a]pyridin-3-yl)ethane-1,2-diamine

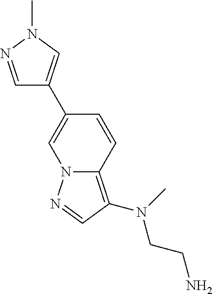 [SMILES: CN(CCN)c1cnn2cc(-c3cnn(C)c3)ccc12]

To a solution of tert-butyl (2-((6-(1-methyl-1H-pyrazol-4-yl)pyrazolo[1,5-a]pyridin-3-yl)amino)ethyl)carbamate (400 mg, 1.12 mmol, as prepared in Example 99, Step 1) dissolved in MeOH (10 mL) and AcOH (5 mL) was added formaldehyde (67.4 mg, 2.24 mmol). The resulting mixture was stirred for 30 min at rt under nitrogen atmosphere. To the above mixture was added NaBH(OAc)₃ (2.38 g, 11.22 mmol) in small portions over 5 min at 0° C. The resulting mixture was stirred for additional 2 h at rt. The precipitated solids were collected by filtration and washed with DCM (3×50 mL) then dried under reduced pressure to afford 220 mg (69%) of N¹-methyl-N¹-(6-(1-methyl-1H-pyrazol-4-yl)pyrazolo[1,5-a]pyridin-3-yl)ethane-1,2-diamine as a yellow solid: MS (ESI) m/z [M+H]⁺ calcd. for C₁₄H₁₈N₆, 271.2; found, 271.2.

### Step 2. Preparation of N¹-(5-benzylpyrimidin-2-yl)-N²-methyl-N²-(6-(1-methyl-1H-pyrazol-4-yl)pyrazolo[1,5-a]pyridin-3-yl)ethane-1,2-diamine (Compound 125)

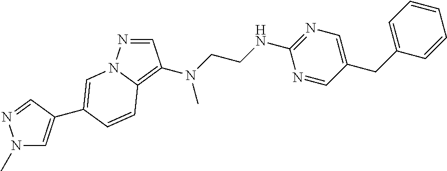 [SMILES: CN(CCNc1ncc(Cc2ccccc2)cn1)c1cnn2cc(-c3cnn(C)c3)ccc12]

To a solution of N¹-methyl-N¹-(6-(1-methyl-1H-pyrazol-4-yl)pyrazolo[1,5-a]pyridin-3-yl)ethane-1,2-diamine (200 mg, 0.74 mmol, as prepared in the previous step) was added 5-benzyl-2-chloropyrimidine (Compound S88) (151.4 mg, 0.74 mmol) and DIPEA (286.9 mg, 2.22 mmol). The resulting mixture was stirred for 16 h at 120° C. under nitrogen atmosphere. The mixture was allowed to cool to rt, diluted with water (30 mL), and extracted with EtOAc (3×30 mL). The combined organic layers were washed with brine (3×30 mL), dried over anhydrous Na₂SO₄. After filtration, the filtrate was concentrated under reduced pressure. The crude product was purified by Prep-HPLC to afford 25.2 mg (7%) of N¹-(5-benzylpyrimidin-2-yl)-N²-methyl-N²-(6-(1-methyl-1H-pyrazol-4-yl)pyrazolo[1,5-a]pyridin-3-yl)ethane-1,2-diamine (Compound 125) as a yellow solid. ¹H NMR (400 MHZ, DMSO-d₆) δ (ppm) 8.75 (s, 1H), 8.18 (d, J=4.8 Hz, 3H), 7.94 (s, 1H), 7.74 (s, 1H), 7.61 (d, J=9.2 Hz, 1H), 7.34-7.13 (m, 6H), 7.01 (t, J=5.8 Hz, 1H), 3.87 (s, 3H), 3.74 (s, 2H), 3.50-3.42 (m, 2H), 3.22-3.15 (m, 2H), 2.81 (s, 3H). MS (ESI) m/z [M+H]⁺ calcd. for C₂₅H₂₆N₈, 439.2; found, 439.3; HPLC purity: 254 nm: 95.8%.

### Example 101: Synthesis of Exemplary Compound 126

### N¹-(5-benzylpyrimidin-2-yl)-N¹-methyl-N²-(6-(1-methyl-1H-pyrazol-4-yl)pyrazolo[1,5-a]pyridin-3-yl)ethane-1,2-diamine (Compound 126)

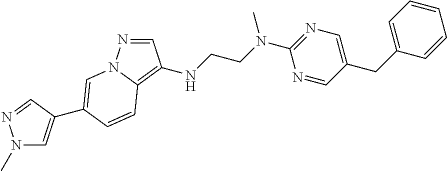 [SMILES: CN(CCNc1cnn2cc(-c3cnn(C)c3)ccc12)c1ncc(Cc2ccccc2)cn1]

### Step 1. Preparation of tert-butyl methyl(2-((6-(1-methyl-1H-pyrazol-4-yl)pyrazolo[1,5-a]pyridin-3-yl)amino)ethyl)carbamate

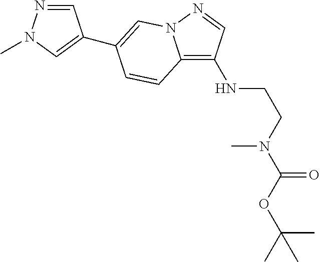 [SMILES: CN(CCNc1cnn2cc(-c3cnn(C)c3)ccc12)C(=O)OC(C)(C)C]

To a solution of 6-(1-methyl-1H-pyrazol-4-yl)pyrazolo[1,5-a]pyridin-3-amine (Compound S56) (500 mg, 2.35 mmol) dissolved in MeOH (20 mL) was added tert-butyl methyl(2-oxoethyl)carbamate (812.3 mg, 4.69 mmol) and NaHCO₃ (591 mg, 7.04 mmol) and the resulting mixture was stirred for 24 h at 60° C. under nitrogen atmosphere. To the above mixture was added NaBH₄ (887.0 mg, 23.45 mmol) over 1 h at 0° C. The reaction was stirred for additional 12 h at rt. The resulting mixture was diluted with water (50 mL) and extracted with EtOAc (3×50 mL). The combined organic layers were washed with brine (3×50 mL), dried over anhydrous Na₂SO₄. After filtration, the filtrate was concentrated under reduced pressure. The residue was purified by Prep-TLC (DCM/MeOH 20:1) to afford 600 mg (62%) of tert-butyl methyl(2-((6-(1-methyl-1H-pyrazol-4-yl)pyrazolo[1,5-a]pyridin-3-yl)amino)ethyl)carbamate as a brown oil. ¹H NMR (400 MHZ, DMSO-d₆) δ (ppm) 8.70 (s, 1H), 8.20 (s, 1H), 7.96 (s, 1H), 7.67-7.52 (m, 2H), 7.17 (dd, J=9.6, 1.6 Hz, 1H), 4.97-4.62 (m, 1H), 3.89 (s, 3H), 3.51-3.37 (m, 2H), 3.24-3.17 (m, 2H), 2.85 (s, 3H), 1.44-1.32 (m, 9H). MS (ESI) m/z [M+H]⁺ calcd. for C₁₉H₂₆N₆O₂, 371.2; found, 371.2.

### Step 2. Preparation of N¹-methyl-N²-(6-(1-methyl-1H-pyrazol-4-yl)pyrazolo[1,5-a]pyridin-3-yl)ethane-1,2-diamine

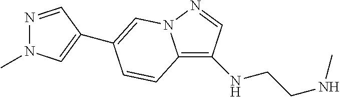

To a solution of tert-butyl methyl(2-((6-(1-methyl-1H-pyrazol-4-yl)pyrazolo[1,5-a]pyridin-3-yl)amino)ethyl)carbamate (700 mg, 1.89 mmol, as prepared in the previous step) dissolved in DCM (10 mL) was added 4 M HCl in dioxane (3 mL) at 0° C. The resulting mixture was stirred for 1 h at 0° C. under nitrogen. The mixture was neutralized to pH 8 with sat. Na₂CO₃ (aq.) then extracted with EtOAc (3×100 mL). The combined organic layers were washed with brine (3×50 mL), dried over anhydrous Na₂SO₄. After filtration, the filtrate was concentrated under reduced pressure to afford 460 mg (81%) of N¹-methyl-N²-(6-(1-methyl-1H-pyrazol-4-yl)pyrazolo[1,5-a]pyridin-3-yl)ethane-1,2-diamine as a yellow solid. MS (ESI) m/z [M+H]⁺ calcd. for C₁₄H₁₈N₆, 271.2; found, 271.1.

### Step 3. Preparation of N¹-(5-benzylpyrimidin-2-yl)-N¹-methyl-N²-(6-(1-methyl-1H-pyrazol-4-yl)pyrazolo[1,5-a]pyridin-3-yl)ethane-1,2-diamine (Compound 126)

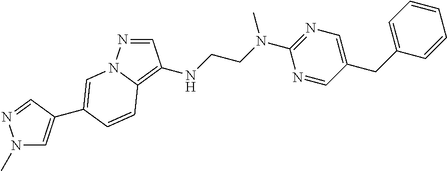 [SMILES: CN(CCNc1cnn2cc(-c3cnn(C)c3)ccc12)c1ncc(Cc2ccccc2)cn1]

To a solution of N¹-methyl-N²-(6-(1-methyl-1H-pyrazol-4-yl)pyrazolo[1,5-a]pyridin-3-yl)ethane-1,2-diamine (200 mg, 0.74 mmol, as prepared in the previous step) in IPA (8 mL) was added 5-benzyl-2-chloropyrimidine (Compound S88) (166.6 mg, 0.81 mmol) and DIPEA (286.9 mg, 2.22 mmol). The resulting mixture was stirred for 24 h at 120° C. under nitrogen. The mixture was allowed to cool to rt. The resulting mixture was diluted with water (50 mL) and extracted with EtOAc (3×30 mL). The combined organic layers were washed with brine (3×30 mL) and dried over anhydrous Na₂SO₄. After filtration, the filtrate was concentrated under reduced pressure. The crude product was purified by Prep-HPLC to afford 70.1 mg (22%) of N¹-(5-benzylpyrimidin-2-yl)-N¹-methyl-N²-(6-(1-methyl-1H-pyrazol-4-yl)pyrazolo[1,5-a]pyridin-3-yl)ethane-1,2-diamine (Compound 126) as a yellow solid. ¹H NMR (400 MHz, DMSO-d₆) δ (ppm) 8.65 (s, 1H), 8.26 (s, 2H), 8.16 (s, 1H), 7.92 (s, 1H), 7.60 (s, 1H), 7.59-7.54 (m, 1H), 7.34-7.15 (m, 5H), 7.11 (dd, J=9.2, 1.2 Hz, 1H), 4.92 (t, J=6.0 Hz, 1H), 3.86 (s, 3H), 3.80-3.72 (m, 4H), 3.29-3.22 (m, 2H), 3.12 (s, 3H). MS (ESI) m/z [M+H]⁺ calcd. for C₂₅H₂₆N₈, 439.2; found, 439.3; HPLC purity: 254 nm: 99.5%.

### Example 102: Synthesis of Exemplary Compound 127

### N¹-(5-benzylpyrimidin-2-yl)-N¹,N²-dimethyl-N²-(6-(1-methyl-1H-pyrazol-4-yl)pyrazolo[1,5-a]pyridin-3-yl)ethane-1,2-diamine (Compound 127)

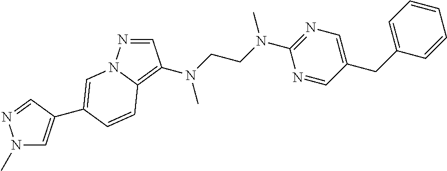 [SMILES: CN(CCN(C)c1cnn2cc(-c3cnn(C)c3)ccc12)c1ncc(Cc2ccccc2)cn1]

To a solution of N¹-(5-benzylpyrimidin-2-yl)-N¹-methyl-N²-(6-(1-methyl-1H-pyrazol-4-yl)pyrazolo[1,5-a]pyridin-3-yl)ethane-1,2-diamine (Compound 126) (100 mg, 0.23 mmol) dissolved in DMF (3 mL) was added CH₃I (48.6 mg, 0.34 mmol) and K₂CO₃ (63.0 mg, 0.46 mmol) while keeping the temperature at 0° C. The resulting mixture was stirred for 3 days at 60° C. under nitrogen atmosphere. The reaction was quenched with MeOH at 0° C. The resulting mixture was concentrated under reduced pressure and purified by Prep-HPLC to afford 38.5 mg (36%) of N¹-(5-benzylpyrimidin-2-yl)-N¹,N²-dimethyl-N²-(6-(1-methyl-1H-pyrazol-4-yl)pyrazolo[1,5-a]pyridin-3-yl)ethane-1,2-diamine (Compound 127) as a yellow semi-solid. ¹H NMR (400 MHZ, DMSO-d₆) δ (ppm) 8.75 (s, 1H), 8.25 (s, 2H), 8.18 (s, 1H), 7.94 (s, 1H), 7.73 (s, 1H), 7.54 (d, J=9.6 Hz, 1H), 7.33-7.14 (m, 6H), 3.88 (s, 3H), 3.79-3.70 (m, 4H), 3.21 (t, J=7.2 Hz, 2H), 3.07 (s, 3H), 2.82 (s, 3H). MS (ESI) m/z [M+H]⁺ calcd. for C₂₆H₂₈N₈, 453.2; found, 453.3; HPLC purity: 254 nm: 96.9%.

### Example 103: Synthesis of Exemplary Compound 128

### N¹-(5-benzylpyrimidin-2-yl)-N³-methyl-N³-(6-(1-methyl-1H-pyrazol-4-yl)pyrazolo[1,5-a]pyridin-3-yl)propane-1,3-diamine (Compound 128)

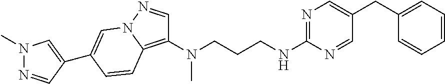 [SMILES: CN(CCCNc1ncc(Cc2ccccc2)cn1)c1cnn2cc(-c3cnn(C)c3)ccc12]

### Step 1. Preparation of N¹-methyl-N¹-(6-(1-methyl-1H-pyrazol-4-yl)pyrazolo[1,5-a]pyridin-3-yl)propane-1,3-diamine

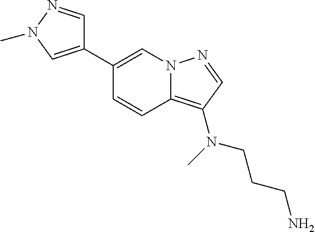 [SMILES: CN(CCCN)c1cnn2cc(-c3cnn(C)c3)ccc12]

To a solution of 3-bromo-6-(1-methyl-1H-pyrazol-4-yl)pyrazolo[1,5-a]pyridine (Compound S9) (350 mg, 1.26 mmol) and N¹-methylpropane-1,3-diamine (134 mg, 1.52 mmol) in tBuOH (4.0 mL)/dioxane (2.0 mL) was added with KOtBu (213 mg, 1.90 mmol) and tBuXPhos Pd G1 (130 mg, 0.189 mmol) under nitrogen. The reaction was stirred for 60 h at 90° C. under nitrogen. The resulting mixture was concentrated under reduced pressure then purified by reversed-phase flash chromatography with C₁₈ silica gel column eluting with 10% to 70% ACN in water (10 mM NH₄HCO₃), to afford N¹-methyl-N¹-(6-(1-methyl-1H-pyrazol-4-yl)pyrazolo[1,5-a]pyridin-3-yl)propane-1,3-diamine (40 mg, 9%) as a yellow solid: MS (ESI) m/z [M+H]⁺ calcd. for C₁₅H₂₀N₆, 285.2; found, 285.3.

### Step 2. Preparation of N¹-(5-benzylpyrimidin-2-yl)-N³-methyl-N³-(6-(1-methyl-1H-pyrazol-4-yl)pyrazolo[1,5-a]pyridin-3-yl)propane-1,3-diamine (Compound 128)

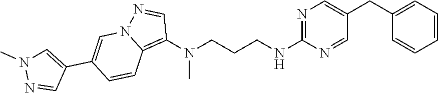 [SMILES: CN(CCCNc1ncc(Cc2ccccc2)cn1)c1cnn2cc(-c3cnn(C)c3)ccc12]

To a solution of N¹-methyl-N¹-(6-(1-methyl-1H-pyrazol-4-yl)pyrazolo[1,5-a]pyridin-3-yl)propane-1,3-diamine (40.0 mg, 0.141 mmol, as prepared in the previous step) and DIPEA (36.4 mg, 0.282 mmol) in IPA (2.0 mL) was added 5-benzyl-2-chloropyrimidine (Compound S88) (28.8 mg, 0.141 mmol). The reaction was stirred for 16 h at 120° C. The resulting mixture was concentrated under reduced pressure then purified by Prep-TLC (PE/EtOAc 1:1). The crude product (50 mg) was purified by Prep-HPLC to afford 13.1 mg (20%) of N¹-(5-benzylpyrimidin-2-yl)-N³-methyl-N³-(6-(1-methyl-1H-pyrazol-4-yl)pyrazolo[1,5-a]pyridin-3-yl)propane-1,3-diamine (Compound 128) as a yellow solid. ¹H NMR (400 MHZ, DMSO-d₆) (ppm) δ 8.76 (s, 1H), 8.19 (s, 1H), 8.15 (s, 2H), 7.94 (s, 1H), 7.72 (s, 1H), 7.55 (d, J=9.2 Hz, 1H), 7.32-7.24 (m, 2H), 7.24-7.14 (m, 4H), 7.04 (t, J=5.7 Hz, 1H), 3.87 (s, 3H), 3.72 (s, 2H), 3.31-3.27 (m, 2H), 3.04 (t, J=7.2 Hz, 2H), 2.74 (s, 3H), 1.77-1.66 (m, 2H). MS (ESI) m/z [M+H]⁺ calcd. for C₂₆H₂₈N₈, 453.2; found, 453.2; HPLC purity: 254 nm: 98.3%.

Using the procedures described in Example 103, Compound 128 and reagents, starting materials, and conditions known to those skilled in the art were used to prepare the compounds shown in Table N.

TABLE N

| Compound | Name | Characterization |
|---|---|---|
| Compound | N¹-(5-benzylpyrimidin-2-yl)-N¹-methyl- | ¹H NMR (400 MHz, DMSO- |
| 129 | N³-(6-(1-methyl-1H-pyrazol-4- | d₆) (ppm) δ 8.67 (s, 1H), 8.25 |
| | yl)pyrazolo[1,5-a]pyridin-3-yl)propane- | (s, 2H), 8.17 (s, 1H), 7.92 (s, |
| | 1,3-diamine | 1H), 7.59 (d, J = 9.2 Hz, 1H), |
| | | 7.50 (s, 1H), 7.32-7.25 (m, |
| | | 2H), 7.24-7.10 (m, 4H), 4.72 |
| | | (t, J = 6.4 Hz, 1H), 3.87 (s, |
| | | 3H), 3.76 (s, 2H), 3.69 (t, J = |
| | | 7.0 Hz, 2H), 3.08 (s, 3H), |
| | | 3.06-2.99 (m, 2H), 1.90-1.76 |
| | | (m, 2H). MS (ESI) m/z |
| | | [M + H]⁺ calcd. for C₂₆H₂₈N₈, |
| | | 453.2; found, 453.2; HPLC |
| | | purity: 254 nm: 99.1%. |
| Compound | N¹-(5-benzylpyrimidin-2-yl)-N¹-methyl- | ¹H NMR (400 MHz, DMSO- |
| 130 | N³-(6-(1-methyl-1H-pyrazol-4- | d₆) (ppm) δ 8.77 (s, 1H), 8.21 |
| | yl)pyrazolo[1,5-a]pyridin-3-yl)propane- | (d, J = 5.7 Hz, 3H), 7.96 (s, |
| | 1,3-diamine | 1H), 7.73 (s, 1H), 7.55 (d, J = |
| | | 9.2 Hz, 1H), 7.31-7.14 (m, |
| | | 6H), 3.88 (s, 3H), 3.74 (s, 2H), |
| | | 3.63 (t, J = 7.2 Hz, 2H), |
| | | 3.04-2.97 (m, 5H), 2.75 (s, 3H), |
| | | 1.81-1.61 (m, 2H). MS |
| | | (ESI) m/z [M + H]⁺ calcd. for |
| | | C₂₇H₃₀N₈, 467.3; found, |
| | | 467.2; HPLC purity: 254 nm: |
| | | 99.8%. |

### Example 104: Synthesis of Exemplary Compound 131

### N-(1-(5-benzylpyrimidin-2-yl) azetidin-3-yl)-6-(1-methyl-1H-pyrazol-4-yl)pyrazolo[1,5-a]pyridin-3-amine (Compound 131)

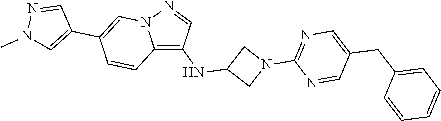 [SMILES: Cn1cc(-c2ccc3c(NC4CN(c5ncc(Cc6ccccc6)cn5)C4)cnn3c2)cn1]

### Step 1. Preparation of tert-butyl (1-(5-benzylpyrimidin-2-yl) azetidin-3-yl)carbamate

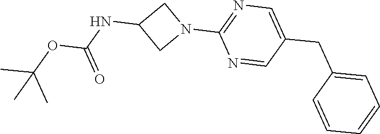 [SMILES: CC(C)(C)OC(=O)NC1CN(c2ncc(Cc3ccccc3)cn2)C1]

To a solution of 5-benzyl-2-chloropyrimidine (Compound S88) (200 mg, 0.977 mmol) and tert-butyl N-(azetidin-3-yl)carbamate hydrochloride (224 mg, 1.08 mmol) in IPA (3.0 mL) was added DIPEA (253 mg, 1.95 mmol). The resulting mixture was stirred for 2 h at 110° C. The mixture was added water (20 mL). The resulting mixture was extracted with EtOAc (3×10 mL). The combined organic layers were dried over anhydrous Na₂SO₄. After filtration, the filtrate was concentrated under reduced pressure to afford 350 mg (94.7%) of tert-butyl (1-(5-benzylpyrimidin-2-yl) azetidin-3-yl)carbamate as a light yellow solid. ¹H NMR (400 MHZ, DMSO-d₆) (ppm) δ 8.26 (s, 2H), 7.56 (d, J=7.1 Hz, 1H), 7.35-7.13 (m, 5H), 4.46-4.32 (m, 1H), 4.18 (t, J=7.7 Hz, 2H), 3.85-3.74 (m, 4H), 1.39 (s, 9H). MS (ESI) m/z [M+H]⁺ calcd. for C₁₉H₂₄N₄O₂, 341.2; found, 341.3.

### Step 2. Preparation of 1-(5-benzylpyrimidin-2-yl) azetidin-3-amine

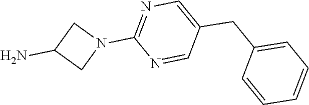 [SMILES: NC1CN(c2ncc(Cc3ccccc3)cn2)C1]

To a solution of tert-butyl (1-(5-benzylpyrimidin-2-yl) azetidin-3-yl)carbamate (640 mg, 1.88 mmol, as prepared in the previous step) in DCM (20.0 mL) was added 4M HCl in dioxane (5.2 mL) dropwise at 0° C. The reaction was stirred for 2 h at rt. The resulting mixture was concentrated under reduced pressure, then DCM was added (20 mL) and concentrated under reduced pressure (twice) and MTBE (20 mL) was added and concentrated under reduced pressure. Water (20 mL) was added, and the mixture was neutralized to pH 11 with sat. aq. Na₂CO₃. The resulting mixture was extracted with MTBE (40 mL). The aqueous layer was saturated with NaCl and extracted with 10% MeOH/DCM (6×20 mL). The combined organic layers were dried over anhydrous Na₂SO₄. After filtration, the filtrate was concentrated under reduced pressure to afford 400 mg (80%) of 1-(5-benzylpyrimidin-2-yl) azetidin-3-amine as a light yellow solid. ¹H NMR (400 MHZ, DMSO-d₆) (ppm) δ 8.23 (s, 2H), 7.33-7.15 (m, 5H), 4.13 (t, J=8.0 Hz, 2H), 3.80-3.70 (m, 3H), 3.62-3.55 (m, 2H), 2.08-2.04 (m, 2H). MS (ESI) m/z [M+H]⁺ calcd. for C₁₄H₁₆N₄, 241.1; found, 241.1.

### Step 3. Preparation of N-(1-(5-benzylpyrimidin-2-yl) azetidin-3-yl)-6-(1-methyl-1H-pyrazol-4-yl)pyrazolo[1,5-a]pyridin-3-amine (Compound 131)

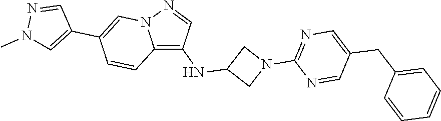 [SMILES: Cn1cc(-c2ccc3c(NC4CN(c5ncc(Cc6ccccc6)cn5)C4)cnn3c2)cn1]

To a solution of 3-bromo-6-(1-methyl-1H-pyrazol-4-yl)pyrazolo[1,5-a]pyridine (Compound S9) (323 mg, 1.17 mmol) and 1-(5-benzylpyrimidin-2-yl) azetidin-3-amine (280 mg, 1.165 mmol, as prepared in the previous step) in tBuOH (4.0 mL) and dioxane (2.0 mL) was added KOtBu (262 mg, 2.33 mmol) and (DiMelHeptCl)Pd(cinnamyl)Cl (123 mg, 0.117 mmol) under nitrogen. The reaction was stirred for 16 h at 90° C. under nitrogen. The resulting mixture was concentrated under reduced pressure. The residue was purified by silica gel column chromatography eluting with PE/EtOAc (1:1). The crude product (150 mg) was purified by Prep-HPLC to afford 45.2 mg (9%) N-(1-(5-benzylpyrimidin-2-yl) azetidin-3-yl)-6-(1-methyl-1H-pyrazol-4-yl)pyrazolo[1,5-a]pyridin-3-amine (Compound 131) as a yellow solid. ¹H NMR (400 MHZ, DMSO-d₆) (ppm) δ 8.78-8.64 (m, 1H), 8.36-8.22 (m, 2H), 8.21-8.09 (m, 1H), 8.01-7.89 (m, 1H), 7.66-7.47 (m, 2H), 7.37-7.14 (m, 6H), 5.52-5.37 (m, 1H), 4.41-4.17 (m, 3H), 3.92-3.71 (m, 7H). MS (ESI) m/z [M+H]⁺ calcd. for C₂₅H₂₄N₈, 437.2; found, 437.2; HPLC purity: 254 nm: 99.4%.

### Example 105: Synthesis of Exemplary Compound 132

### benzyl 4-(6-(1-methyl-1H-pyrazol-4-yl)pyrazolo[1,5-a]pyridin-3-yl)cyclohex-3-ene-1-carboxylate (Compound 132)

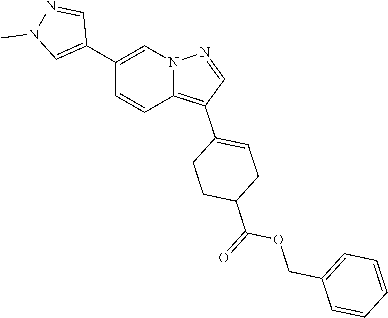 [SMILES: Cn1cc(-c2ccc3c(C4=CCC(C(=O)OCc5ccccc5)CC4)cnn3c2)cn1]

### Step 1. Preparation of benzyl 4-oxocyclohexane-1-carboxylate

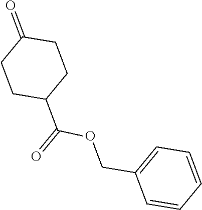 [SMILES: O=C1CCC(C(=O)OCc2ccccc2)CC1]

To a stirred solution of 4-oxocyclohexane-1-carboxylic acid (10 g, 70.35 mmol) and K₂CO₃ (19.44 g, 140.69 mmol) in DMF (150 mL) was added benzyl bromide (13.23 g, 77.38 mmol) at 0° C. The resulting mixture was stirred overnight at rt. The resulting mixture was diluted with water (500 mL) and extracted with DCM (3×100 mL). The combined organic layers were dried over anhydrous Na₂SO₄. After filtration, the filtrate was concentrated under reduced pressure. The residue was purified by reversed-phase flash chromatography with C₁₈ silica gel column eluting with ACN in water (10 mM NH₄HCO₃), 10% to 100% to afford 11 g (66%) of benzyl 4-oxocyclohexane-1-carboxylate as a yellow oil. ¹H NMR (400 MHZ, DMSO-d₆) δ 7.43-7.30 (m, 5H), 5.14 (s, 2H), 2.96-2.83 (m, 1H), 2.47-2.35 (m, 2H), 2.29-2.20 (m, 2H), 2.19-2.10 (m, 2H), 1.84 (m, 2H). MS (ESI) m/z [M+H]⁺ calcd. for C₁₄H₁₆O₃, 233.1; found, 233.2.

### Step 2. Preparation of benzyl 4-(((trifluoromethyl)sulfonyl)oxy)cyclohex-3-ene-1-carboxylate

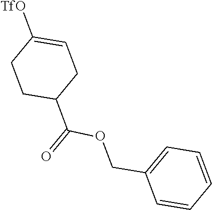 [SMILES: O=C(OCc1ccccc1)C1CC=C(OS(=O)(=O)C(F)(F)F)CC1]

To a stirred solution of benzyl 4-oxocyclohexane-1-carboxylate (3 g, 12.91 mmol, as prepared in the previous step) in THF (60 mL) was added LDA (8.4 mL, 2M) at −78° C. under nitrogen. The resulting mixture was stirred for 0.5 h at −78° C. under nitrogen. To the above mixture was added 1,1,1-trifluoro-N-phenyl-N-((trifluoromethyl)sulfonyl) methanesulfonamide (5.08 g, 14.21 mmol) at −78° C. The reaction was stirred overnight at rt. The reaction was quenched by the addition of sat. aq. NH₄Cl solution at 0° C. The combined organic layers were dried over anhydrous Na₂SO₄. After filtration, the filtrate was concentrated under reduced pressure. The residue was purified by silica gel column chromatography eluting with PE/EtOAc (5:1) to afford 1.5 g (32%) of benzyl 4-(((trifluoromethyl)sulfonyl)oxy)cyclohex-3-ene-1-carboxylate as a yellow oil. ¹H NMR (400 MHz, DMSO-d₆) δ 7.41-7.29 (m, 5H), 5.90 (t, J=3.8 Hz, 1H), 5.17-5.09 (m, 2H), 2.78-2.67 (m, 1H), 2.48-2.26 (m, 4H), 2.12-2.01 (m, 1H), 1.91-1.77 (m, 1H). MS (ESI) m/z [M−H]⁺ calcd. for C₁₅H₁₅F₃O₅S, 363.1; found, 363.2.

### Step 3. Preparation of benzyl 4-(6-(1-methyl-1H-pyrazol-4-yl)pyrazolo[1,5-a]pyridin-3-yl)cyclohex-3-ene-1-carboxylate (Compound 132)

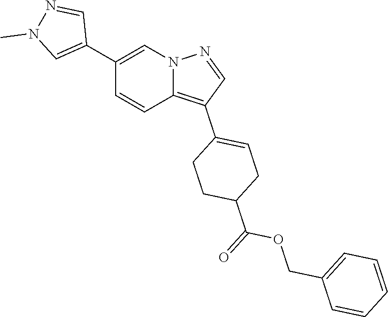 [SMILES: Cn1cc(-c2ccc3c(C4=CCC(C(=O)OCc5ccccc5)CC4)cnn3c2)cn1]

To a stirred solution of benzyl 4-(((trifluoromethyl)sulfonyl)oxy)cyclohex-3-ene-1-carboxylate (500 mg, 1.37 mmol, as prepared in the previous step) and 6-(1-methyl-1H-pyrazol-4-yl)-3-(4,4,5,5-tetramethyl-1,3,2-dioxaborolan-2-yl)pyrazolo[1,5-a]pyridine (Compound S57) (355.9 mg, 1.10 mmol) in dioxane (15 mL) and H₂O (1.5 mL) were added XPhos (130.9 mg, 0.27 mmol), XPhos Pd G3 (116.2 mg, 0.14 mmol) and K₃PO₄ (582.6 mg, 2.74 mmol) at rt. The reaction was stirred for 2 h at 90° C. under nitrogen. The resulting mixture was extracted with DCM (3×100 mL). The combined organic layers were dried over anhydrous Na₂SO₄. After filtration, the filtrate was concentrated under reduced pressure. The residue was purified by silica gel column chromatography eluting with DCM/MeOH (15:1) and then purified by Prep-HPLC to afford 71.7 mg (13%) of benzyl 4-(6-(1-methyl-1H-pyrazol-4-yl)pyrazolo[1,5-a]pyridin-3-yl)cyclohex-3-ene-1-carboxylate (Compound 132) as a yellow solid. ¹H NMR (400 MHZ, DMSO-d₆) δ (ppm) 8.95 (s, 1H), 8.26 (s, 1H), 8.01 (d, J=10.2 Hz, 2H), 7.82 (d, J=9.3 Hz, 1H), 7.51-7.45 (m, 1H), 7.42-7.30 (m, 5H), 6.14-6.06 (m, 1H), 5.16 (s, 2H), 3.88 (s, 3H), 2.80-2.66 (m, 1H), 2.62-2.53 (m, 2H), 2.49-2.31 (m, 2H), 2.18-2.05 (m, 1H), 1.87-1.70 (m, 1H). MS (ESI) m/z [M+H]⁺ calcd. for C₂₅H₂₄N₄O₂, 413.2; found, 413.1; HPLC purity: 254 nm: 99.4%.

### Example 106: Synthesis of Exemplary Compound 133

### benzyl 3-(6-(1-methyl-1H-pyrazol-4-yl)pyrazolo[1,5-a]pyridin-3-yl)cyclopentane-1-carboxylate (Compound 133)

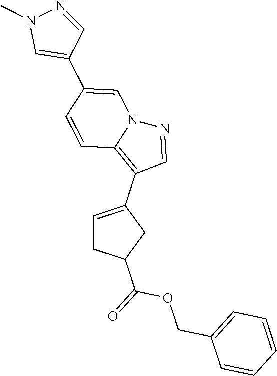 [SMILES: Cn1cc(-c2ccc3c(C4=CCC(C(=O)OCc5ccccc5)C4)cnn3c2)cn1]

### Step 1. Preparation of benzyl 3-oxocyclopentane-1-carboxylate

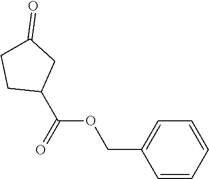 [SMILES: O=C1CCC(C(=O)OCc2ccccc2)C1]

To a solution of 3-oxocyclopentane-1-carboxylic acid (200 mg, 1.56 mmol) and K₂CO₃ (431.5 mg, 3.12 mmol) in DMF (5 mL) were added benzyl bromide (293.7 mg, 1.72 mmol). After stirring for 16 h at rt under nitrogen, the resulting mixture was concentrated under reduced pressure. The residue was purified by Prep-TLC, eluting with PE/EtOAc (3:1) to afford 105 mg (31%) of benzyl 3-oxocyclopentane-1-carboxylate as a colorless oil. ¹H NMR (400 MHZ, DMSO-d₆) δ (ppm) 7.54-7.17 (m, 5H), 5.14 (s, 2H), 3.30-3.13 (m, 1H), 2.47-2.14 (m, 5H), 2.08-1.90 (m, 1H).

### Step 2. benzyl 3-(trifluoromethanesulfonyloxy)cyclopent-3-ene-1-carboxylate

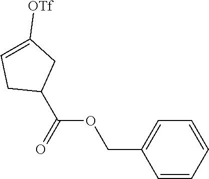 [SMILES: O=C(OCc1ccccc1)C1CC=C(OS(=O)(=O)C(F)(F)F)C1]

To a solution of benzyl 3-oxocyclopentane-1-carboxylate (200 mg, 0.92 mmol, as prepared in the previous step) in THF (20 mL) was added LDA (127.6 mg, 1.19 mmol) at −78° C. After stirring for 0.5h at −78° C. under nitrogen atmosphere, 1,1,1-trifluoro-N-phenyl-N-trifluoromethanesulfonylmethanesulfonamide (360.1 mg, 1.01 mmol) was added dropwise at −78° C. under nitrogen. After stirring for 16 h at rt, the reaction was quenched with sat. aq. NH₄Cl solution at 0° C. then concentrated under reduced pressure. The residue was purified by Prep-TLC, eluting with PE/EtOAc (3:1) to afford 90 mg (28%) of benzyl 3-(trifluoromethanesulfonyloxy)cyclopent-3-ene-1-carboxylate as a yellow oil. ¹H NMR (400 MHz, DMSO-d₆) δ (ppm) 7.46-7.30 (m, 5H), 5.96-5.84 (m, 1H), 5.15 (s, 2H), 3.85-3.40 (m, 1H), 2.93-2.84 (m, 1H), 2.80-2.56 (m, 2H), 2.38-2.16 (m, 1H).

### Step 3. Preparation of benzyl 3-[6-(1-methylpyrazol-4-yl)pyrazolo[1,5-a]pyridin-3-yl]cyclopent-3-ene-1-carboxylate (Compound 133)

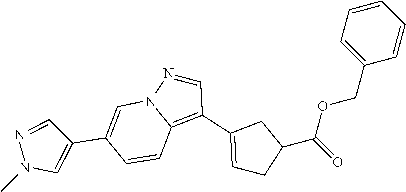 [SMILES: Cn1cc(-c2ccc3c(C4=CCC(C(=O)OCc5ccccc5)C4)cnn3c2)cn1]

To a solution of 6-(1-methyl-1H-pyrazol-4-yl)-3-(4,4,5,5-tetramethyl-1,3,2-dioxaborolan-2-yl)pyrazolo[1,5-a]pyridine (Compound S57) (300 mg, 0.93 mmol), benzyl 3-(trifluoromethanesulfonyloxy)cyclopent-3-ene-1-carboxylate (324.2 mg, 0.93 mmol, as prepared in the previous step), XPhos (88.2 mg, 0.19 mmol), and K₃PO₄ (589.3 mg, 2.78 mmol) in dioxane (20 mL) was added XPhos Pd G3 (78.3 mg, 0.093 mmol) at rt. After stirring for 16 h at 90° C. under nitrogen, the mixture was allowed to cool to rt. then concentrated under reduced pressure. The residue was purified by Prep-TLC, eluting with PE/EtOAc (1:1) and The crude product was purified by Prep-HPLC to afford 60.9 mg (17%) of benzyl 3-[6-(1-methylpyrazol-4-yl)pyrazolo[1,5-a]pyridin-3-yl]cyclopent-3-ene-1-carboxylate (Compound 133) as a grey semi-solid. ¹H NMR (400 MHz, DMSO-d₆) δ (ppm) 8.99 (d, J=6.0 Hz, 1H), 8.27 (d, J=2.2 Hz, 1H), 8.11-8.06 (m, 1H), 8.02 (s, 1H), 7.88 (t, J=8.6 Hz, 1H), 7.60-7.51 (m, 1H), 7.43-7.31 (m, 5H), 6.09-6.00 (m, 1H), 5.16 (d, J=3.0 Hz, 2H), 3.88 (s, 3H), 3.41-3.36 (m, 1H), 3.11-3.06 (m, 1H), 2.93-2.75 (m, 2H), 2.28-2.23 (m, 1H) MS (ESI) m/z [M+H]⁺ calcd. For C₂₄H₂₂N₄O₂, 399.2; found, 399.3; HPLC purity: 254 nm: 97.9%.

### Example 107: Synthesis of Exemplary Compound 134

### tert-butyl 4-(6-(pyrimidin-5-yl)pyrazolo[1,5-a]pyridin-3-yl)piperazine-1-carboxylate (Compound 134)

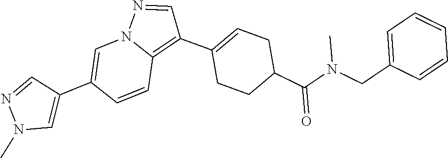 [SMILES: CN(Cc1ccccc1)C(=O)C1CC=C(c2cnn3cc(-c4cnn(C)c4)ccc23)CC1]

### Step 1. Preparation of N-benzyl-N-methyl-4-oxocyclohexane-1-carboxamide

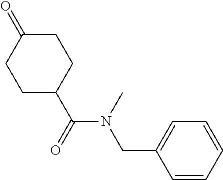 [SMILES: CN(Cc1ccccc1)C(=O)C1CCC(=O)CC1]

To a solution of 4-oxocyclohexanecarboxylate (10.0 g, 70.3 mmol) in DCM (300 mL) was added DIPEA (27.3 g, 211 mmol), N-methylbenzylamine (8.52 g, 70.3 mmol) and HATU (53.5 g, 141 mmol) in portions at 0° C. The reaction was stirred for 16 h at rt. The resulting mixture was filtered, the filter cake was washed with DCM (3×10 mL). The filtrate was concentrated under reduced pressure. The residue was purified by silica gel column chromatography eluting with PE/EtOAc (1:1) then further purified by reversed-phase flash chromatography C₁₈ silica gel column eluting ACN in water (10 mMNH₄HCO₃), 10% to 60% to afford 14 g (73%) of N-benzyl-N-methyl-4-oxocyclohexane-1-carboxamide as an orange oil. ¹H NMR (400 MHZ, DMSO-d₆) δ (ppm) 7.46-7.15 (m, 5H), 4.71 (s, 1H), 4.53 (s, 1H), 3.24-3.11 (m, 1H), 3.03 (s, 2H), 2.80 (s, 1H), 2.51-2.37 (m, 2H), 2.33-2.16 (m, 2H), 2.10-1.88 (m, 2H), 1.86-1.71 (m, 2H). MS (ESI) m/z [M+H]⁺ calcd. for C₁₅H₁₉NO₂, 246.1; found, 246.2.

### Step 2. Preparation of 4-(benzyl(methyl)carbamoyl)cyclohex-1-en-1-yl trifluoromethanesulfonate

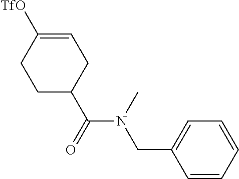 [SMILES: CN(Cc1ccccc1)C(=O)C1CC=C(OS(=O)(=O)C(F)(F)F)CC1]

To a solution of N-benzyl-N-methyl-4-oxocyclohexane-1-carboxamide (2.00 g, 8.15 mmol, as prepared in the previous step) in THF (40.0 mL) was treated with LDA (1.31 g, 12.2 mmol) for 1 h at −78° C. under nitrogen atmosphere followed by the addition of 1,1,1-trifluoro-N-phenyl-N-trifluoromethanesulfonylmethanesulfonamide (3.20 g, 8.96 mmol) in THF (10.0 mL) dropwise at −78° C. The resulting mixture was stirred for 1 h at rt under nitrogen atmosphere. The reaction was quenched by the addition of sat. aq. NH₄Cl (40 mL) at 0° C. The resulting mixture was extracted with EtOAc (2×100 mL). Combined organic layers were dried over anhydrous Na₂SO₄. After filtration, the filtrate was concentrated under reduced pressure. The residue was purified by reversed-phase flash chromatography with C18 silica gel column eluting with ACN in water (10 mM NH₄HCO₃) 10% to 70% to afford 1.2 g (35%) of 4-(benzyl(methyl)carbamoyl)cyclohex-1-en-1-yl trifluoromethanesulfonate as a yellow oil. ¹H NMR (400 MHZ, DMSO-d₆) δ (ppm) 7.49-7.06 (m, 5H), 6.05-5.77 (m, 1H), 4.86-4.35 (m, 2H), 2.99 (s, 2H), 2.97-2.84 (m, 1H), 2.82 (s, 1H), 2.47-2.26 (m, 3H), 2.27-1.79 (m, 2H), 1.79-1.62 (m, 1H). MS (ESI) m/z [M+H]⁺ calcd. for C₁₆H₁₈F₃NO₄S, 378.1; found, 378.1.

### Step 3. Preparation of N-benzyl-N-methyl-4-(6-(1-methyl-1H-pyrazol-4-yl)pyrazolo[1,5-a]pyridin-3-yl)cyclohex-3-ene-1-carboxamide (Compound 134)

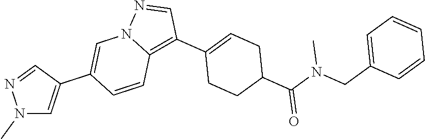 [SMILES: CN(Cc1ccccc1)C(=O)C1CC=C(c2cnn3cc(-c4cnn(C)c4)ccc23)CC1]

To a solution of 4-(benzyl(methyl)carbamoyl)cyclohex-1-en-1-yl trifluoromethanesulfonate (866 mg, 2.29 mmol, as prepared in the previous step) and 6-(1-methyl-1H-pyrazol-4-yl)-3-(4,4,5,5-tetramethyl-1,3,2-dioxaborolan-2-yl)pyrazolo[1,5-a]pyridine (Compound S57) (620 mg, 1.91 mmol) in dioxane (10.0 mL) and H₂O (1.0 mL) was added XPhos Pd G₃ (324 mg, 0.382 mmol), XPhos (182 mg, 0.382 mmol) and K₃PO₄ (812 mg, 3.82 mmol) under nitrogen. The resulting mixture was stirred for 16 h at 90° C. under nitrogen. The resulting mixture was filtered, the filter cake was washed with EtOAc (2×10 mL). The filtrate was concentrated under reduced pressure. The residue was purified by silica gel column chromatography eluting with PE/EtOAc (1:2). The crude product was purified by Prep-HPLC to afford 54.0 mg (7%) of N-benzyl-N-methyl-4-(6-(1-methyl-1H-pyrazol-4-yl)pyrazolo[1,5-a]pyridin-3-yl)cyclohex-3-ene-1-carboxamide (Compound 134) as a light yellow solid. ¹H NMR (400 MHZ, DMSO-d₆) δ (ppm) 8.94 (d, J=8.8 Hz, 1H), 8.25 (d, J=4.4 Hz, 1H), 8.09-7.93 (m, 2H) 7.89-7.78 (m, 1H), 7.51-7.25 (m, 4H), 7.25-7.19 (m, 2H), 6.29-5.87 (m, 1H), 4.80-4.48 (m, 2H), 3.87 (d, J=2.8 Hz, 3H), 3.08-2.82 (m, 4H), 2.71-2.54 (m, 2H), 2.47-2.17 (m, 2H), 2.02-1.81 (m, 1H), 1.80-1.60 (m, 1H). MS (ESI) m/z [M+H]⁺ calcd. for C₂₆H₂₇N₅O, 426.2; found, 426.2; HPLC purity: 254 nm: 98.5%.

Using the procedures described in Example 107, Compound 134 and reagents, starting materials, and conditions known to those skilled in the art were used to prepare the compounds shown in Table O.

TABLE O

| Compound | Name | Characterization |
|---|---|---|
| Compound | N-benzyl-N-methyl-3-(6-(1-methyl-1H- | ¹H NMR (400 MHz, DMSO- |
| 135 | pyrazol-4-yl)pyrazolo[1,5-a]pyridin-3- | d₆) δ (ppm) 8.99 (t, J = 5.0 Hz, |
| | yl)cyclopent-3-ene-1-carboxamide | 1H), 8.28 (s, 1H), 8.14-7.97 |
| | | (m, 2H), 7.95-7.65 (m, 1H), |
| | | 7.60-7.18 (m, 6H), 6.21-5.62 |
| | | (m, 1H), 4.95-4.33 (m, |
| | | 2H), 4.17-3.54 (m, 4H), |
| | | 3.16-2.64 (m, 6H), 2.28-2.08 |
| | | (m, 1H). MS (ESI) m/z |
| | | [M + H]⁺ calcd. for C₂₅H₂₅N₅O, |
| | | 412.2; found, 412.1; HPLC |
| | | purity: 254 nm: 98.1%. |

### Example 108: Synthesis of Exemplary Compound 136

### benzyl 4-(6-(1-methyl-1H-pyrazol-4-yl)pyrazolo[1,5-a]pyridin-3-yl)cyclohexane-1-carboxylate (Compound 136)

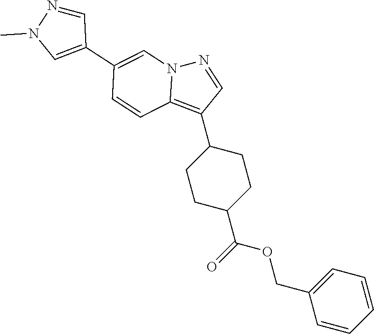 [SMILES: Cn1cc(-c2ccc3c(C4CCC(C(=O)OCc5ccccc5)CC4)cnn3c2)cn1]

### Step 1. Preparation of 4-(6-(1-methyl-1H-pyrazol-4-yl)pyrazolo[1,5-a]pyridin-3-yl)cyclohexane-1-carboxylic acid

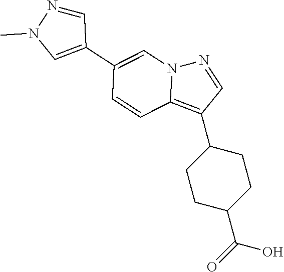 [SMILES: Cn1cc(-c2ccc3c(C4CCC(C(=O)O)CC4)cnn3c2)cn1]

To a stirred solution of benzyl 4-(6-(1-methyl-1H-pyrazol-4-yl)pyrazolo[1,5-a]pyridin-3-yl)cyclohex-3-ene-1-carboxylate (Compound 132) (640 mg, 1.55 mmol) in MeOH (10 mL) was added Pd/C (33 mg, 0.31 mmol) at rt. The resulting mixture was stirred for 3 days at rt under hydrogen atmosphere. The resulting mixture was filtered, the filter cake was washed with DCM (3×80 mL). The filtrate was concentrated under reduced pressure. The residue was purified by reversed-phase flash chromatography C18 silica gel column eluting with 10% to 100% ACN in Water (10 mM NH₄HCO₃) to afford 160 mg (32%) of 4-(6-(1-methyl-1H-pyrazol-4-yl)pyrazolo[1,5-a]pyridin-3-yl)cyclohexane-1-carboxylic acid as a white solid. ¹H NMR (400 MHZ, DMSO-d₆) δ (ppm) 12.16 (s, 1H), 8.89 (s, 1H), 8.22 (s, 1H), 7.97 (s, 1H), 7.78 (s, 1H), 7.65 (d, J=9.2 Hz, 1H), 7.39 (dd, J=9.2, 1.6 Hz, 1H), 3.87 (s, 3H), 2.97-2.83 (m, 1H), 2.65-2.58 (m, 1H), 2.16-1.95 (m, 2H), 1.86-1.57 (m, 6H). MS (ESI) m/z [M+H]⁺ calcd. for C₁₈H₂₀N₄O₂, 325.2; found, 325.3.

### Step 2. Preparation of benzyl 4-(6-(1-methyl-1H-pyrazol-4-yl)pyrazolo[1,5-a]pyridin-3-yl)cyclohexane-1-carboxylate (Compound 136)

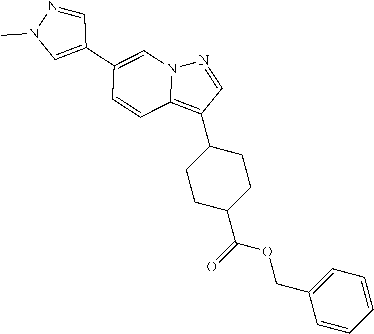 [SMILES: Cn1cc(-c2ccc3c(C4CCC(C(=O)OCc5ccccc5)CC4)cnn3c2)cn1]

To a solution of 4-(6-(1-methyl-1H-pyrazol-4-yl)pyrazolo[1,5-a]pyridin-3-yl)cyclohexane-1-carboxylic acid (150 mg, 0.46 mmol, as prepared in the previous step) and K₂CO₃ (127.8 mg, 0.92 mmol) in DMF (3 mL) was added benzyl bromide (87 mg, 0.51 mmol) at 0° C. The reaction was stirred for 1 h at rt. The resulting mixture was filtered, the filter cake was washed with DCM (3×50 mL). The filtrate was concentrated under reduced pressure. The crude product was purified by Prep-HPLC to afford 124.6 mg (65%) of benzyl 4-(6-(1-methyl-1H-pyrazol-4-yl)pyrazolo[1,5-a]pyridin-3-yl)cyclohexane-1-carboxylate (Compound 136) as a white solid. ¹H NMR (400 MHZ, DMSO-d₆) δ (ppm) 8.88 (s, 1H), 8.23 (s, 1H), 7.97 (s, 1H), 7.72 (s, 1H), 7.59 (d, J=9.2 Hz, 1H), 7.48-7.26 (m, 6H), 5.17 (s, 2H), 3.88 (s, 3H), 2.94-2.85 (m, 1H), 2.84-2.75 (m, 1H), 2.17-2.03 (m, 2H), 1.87-1.52 (m, 6H). MS (ESI) m/z [M+H]⁺ calcd. for C₂₅H₂₆N₄O₂, 415.2; found, 415.15; HPLC purity: 254 nm: 99.4%.

Using the procedures described in Example 109, Compound 136 and reagents, starting materials, and conditions known to those skilled in the art were used to prepare the compounds shown in Table P.

TABLE P

| Compound | Name | Characterization |
|---|---|---|
| Compound | benzyl 3-(6-(1-methyl-1H-pyrazol-4- | ¹H NMR (400 MHz, DMSO- |
| 137 | yl)pyrazolo[1,5-a]pyridin-3- | d₆) δ (ppm) 8.90 (t, J = 1.2 Hz, |
| | yl)cyclopentane-1-carboxylate | 1H), 8.23 (s, 1H), 7.98 (d, J = |
| | | 0.8 Hz, 1H), 7.84 (s, 1H), |
| | | 7.70-7.65 (m, 1H), 7.43-7.30 |
| | | (m, 6H), 5.13 (s, 2H), 3.87 (s, |
| | | 3H), 3.33-3.23(m, 1H), 3.12- |
| | | 2.99(m, 1H), 2.48-2.39 (m, |
| | | 1H), 2.19-1.96 (m, 3H), 1.91- |
| | | 1.82 (m, 1H), 1.82-1.66 |
| | | (m, 1H). MS (ESI) m/z |
| | | [M + H]⁺ calcd. for |
| | | C₂₄H₂₄N₄O₂, 401.2; found, |
| | | 401.3; HPLC purity: 254 nm: |
| | | 99.7%. |

### Example 109: Synthesis of Exemplary Compound 138

### 6-(1-methyl-1H-pyrazol-4-yl)-3-(4-(phenoxymethyl)cyclohexyl)pyrazolo[1,5-a]pyridine (Compound 138)

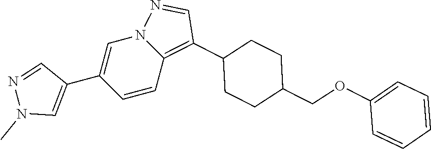 [SMILES: Cn1cc(-c2ccc3c(C4CCC(COc5ccccc5)CC4)cnn3c2)cn1]

To a stirred solution of 1-methyl-4-{3-[4-(phenoxymethyl)cyclohex-1-en-1-yl]pyrazolo[1,5-a]pyridin-6-yl}pyrazole (Compound 243) (200 mg, 0.52 mmol) in MeOH (50 mL, 741 mmol) was added Pd/C (110.71 mg, 1.04 mmol) in portions at rt under nitrogen. The resulting mixture was stirred for 2 h at rt under hydrogen atmosphere. The resulting mixture was filtered, the filter cake was washed with MeOH (2×10 mL). The filtrate was concentrated under reduced pressure. The crude product was purified by Prep-HPLC to afford 50.9 mg (25%) of 6-(1-methyl-1H-pyrazol-4-yl)-3-(4-(phenoxymethyl)cyclohexyl)pyrazolo[1,5-a]pyridine (Compound 138) as a white solid. ¹H NMR (400 MHZ, DMSO-d₆) δ (ppm) 8.89 (s, 1H), 8.23 (s, 1H), 7.97 (s, 1H), 7.92-7.80 (m, 1H), 7.70 (t, J=9.8 Hz, 1H), 7.38 (dd, J=9.3, 1.6 Hz, 1H), 7.30-7.26 (m, 2H), 7.01-6.87 (m, 3H), 4.05-3.82 (m, 5H), 3.11-2.76 (m, 1H), 2.18-2.06 (m, 1H), 2.02-1.84 (m, 1H), 1.82-1.71 (m, 5H), 1.61-1.51 (m, 1H), 1.32-1.20 (m, 1H). MS (ESI) m/z [M+H]⁺ calcd. for C₂₄H₂₆N₄O, 387.1; found, 387.2. HPLC purity: 254 nm: 99.9%.

Using the procedures described in Example 109, Compound 138 and reagents, starting materials, and conditions known to those skilled in the art were used to prepare the compounds shown in Table Q.

TABLE Q

| Compound | Name | Characterization |
|---|---|---|
| Compound | N-benzyl-N-methyl-4-(6-(1-methyl-1H- | ¹H NMR (400 MHz, DMSO- |
| 139 | pyrazol-4-yl)pyrazolo[1,5-a]pyridin-3- | d₆) δ (ppm) 8.91 (s, 1H), 8.23 |
| | yl)cyclohexane-1-carboxamide | (s, 1H), 7.98 (s, 1H), 7.86 (s, |
| | | 1H), 7.73-7.64 (m, 1H), 7.45- |
| | | 7.15 (m, 6H), 4.64 (s, 1H), |
| | | 4.51 (s, 1H), 3.87 (s, 3H), 3.12- |
| | | 3.03 (m, 1H), 3.02-2.82 |
| | | (m, 3H), 2.79 (s, 1H), 2.17- |
| | | 2.00 (m, 2H), 1.88-1.73 (m, |
| | | 4H), 1.71-1.50 (m, 2H). MS |
| | | (ESI) m/z [M + H]⁺ calcd. for |
| | | C₂₆H₂₉N₅O, 428.2; found, |
| | | 428.25; HPLC purity: 254 nm: |
| | | 99.0%. |
| Compound | N-benzyl-N-methyl-3-(6-(1-methyl-1H- | ¹H NMR (400 MHz, DMSO- |
| 140 | pyrazol-4-yl)pyrazolo[1,5-a]pyridin-3- | d₆) (ppm) δ 8.89 (d, J = 4.4 |
| | yl)cyclopentane-1-carboxamide | Hz, 1H), 8.22 (s, 1H), 7.98 (s, |
| | | 1H), 7.86 (d, J = 8.0 Hz, 1H), |
| | | 7.77-7.63 (m, 1H), 7.47- |
| | | 7.15 (m, 6H), 4.69 (s, 1H), |
| | | 4.55 (s, 1H), 3.88 (s, 3H), 3.33- |
| | | 3.15 (m, 2H), 2.99 (s, 2H), |
| | | 2.84 (s, 1H), 2.43-2.20 (m, |
| | | 1H), 2.16-2.03 (m, 1H), 2.02- |
| | | 1.83 (m, 3H), 1.83-1.70 |
| | | (m, 1H). MS (ESI) m/z |
| | | [M + H]+ calcd. for C₂₅H₂₇N₅O, |
| | | 414.2; found, 414.20; HPLC |
| | | purity: 254 nm: 99.4%. |

### Example 110: Synthesis of Exemplary Compound 141

### 6-(1-methyl-1H-pyrazol-4-yl)-3-(4-phenethylcyclohexyl)pyrazolo[1,5-a]pyridine (Compound 141)

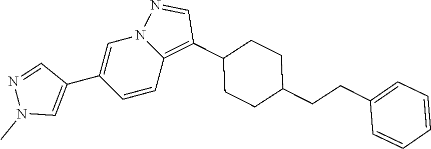 [SMILES: Cn1cc(-c2ccc3c(C4CCC(CCc5ccccc5)CC4)cnn3c2)cn1]

To a solution of 6-(1-methyl-1H-pyrazol-4-yl)-3-(4-phenethylcyclohex-1-en-1-yl)pyrazolo[1,5-a]pyridine (Compound 242) (200 mg, 0.52 mmol) in EtOAc (30 mL) was added Pd/C (55 mg, 0.52 mmol). The reaction was stirred for 16 h at rt under hydrogen atmosphere. The resulting mixture was filtered, the filter cake was washed with EtOAc (2×10 mL). The filtrate was concentrated under reduced pressure. The crude product was purified by Prep-HPLC to afford 82.1 mg (41%) of 6-(1-methyl-1H-pyrazol-4-yl)-3-(4-phenethylcyclohexyl)pyrazolo[1,5-a]pyridine (Compound 141) as a white solid. ¹H NMR (400 MHz, DMSO-d₆) δ (ppm) 8.88 (s, 1H), 8.22 (s, 1H), 7.97 (s, 1H), 7.89-7.80 (m, 1H), 7.67 (t, J=4.2 Hz, 1H), 7.38-7.13 (m, 6H), 3.87 (s, 3H), 2.96-2.77 (m, 1H), 2.63 (m, 2H), 1.92 (t, J=6.4 Hz, 2H), 1.83-1.64 (m, 5H), 1.61-1.42 (m, 3H), 1.34 (m, 1H). MS (ESI) m/z [M−H]⁺ calcd. for C₂₅H₂₈N₄, 385.2; found, 385.1. HPLC purity: 254 nm: 98.6%.

### Example 111: Synthesis of Exemplary Compound 142

### 3-(4-(5-benzylpyrimidin-2-yl)-2-methylpiperazin-1-yl)-6-(1-methyl-1H-pyrazol-4-yl)pyrazolo[1,5-a]pyridine (Compound 142)

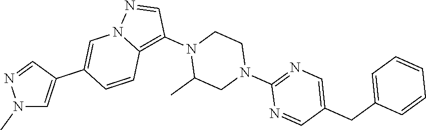 [SMILES: CC1CN(c2ncc(Cc3ccccc3)cn2)CCN1c1cnn2cc(-c3cnn(C)c3)ccc12]

### Step 1. Preparation of tert-butyl-3-methyl-4-(6-(1-methyl-1H-pyrazol-4-yl)pyrazolo[1,5-a]pyridin-3-yl)piperazine-1-carboxylate

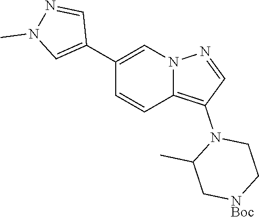 [SMILES: CC1CN(C(=O)OC(C)(C)C)CCN1c1cnn2cc(-c3cnn(C)c3)ccc12]

To a solution of 3-bromo-6-(1-methyl-1H-pyrazol-4-yl)pyrazolo[1,5-a]pyridine (Compound S9) (3.00 g, 10.90 mmol) dissolved in toluene (60.0 mL) was added tert-butyl-3-methylpiperazine-1-carboxylate (2.60 g, 13.00 mmol), KOtBu (2.43 g, 21.60 mmol), tBuXPhos (459 mg, 1.08 mmol) and tBuXPhos Pd G1 (861 mg, 1.08 mmol). The resulting solution was stirred 90° C. for 16 h under nitrogen. The mixture was allowed to cool to rt. The residue was applied onto a silica gel column with EtOAc/PE (1/1). The crude product was purified by Prep-HPLC to afford 93.0 mg (2%) of tert-butyl-3-methyl-4-(6-(1-methyl-1H-pyrazol-4-yl)pyrazolo[1,5-a]pyridin-3-yl)piperazine-1-carboxylate as a light yellow oil. ¹H NMR (400 MHZ, DMSO-d₆) δ (ppm) 8.84 (s, 1H), 8.22 (s, 1H), 7.97 (s, 1H), 7.91 (s, 1H), 7.60 (d, J=9.2 Hz, 1H), 7.36 (d, J=9.3 Hz, 1H), 3.87 (s, 3H), 3.81 (s, 1H), 3.71 (d, J=12.8 Hz, 1H), 3.25-3.20 (m, 1H), 3.05 (s, 2H), 2.92 (d, J=9.8 Hz, 2H), 1.44 (s, 9H) 0.81 (d, J=6.0 Hz, 3H). MS (ESI) m/z [M+H]⁺ calcd. for C₂₁H₁₈N₆O₂, 397.2; found, 397.2.

### Step 2. Preparation of 6-(1-methyl-1H-pyrazol-4-yl)-3-(2-methylpiperazin-1-yl)pyrazolo[1,5-a]pyridine

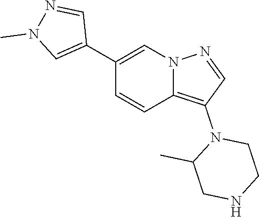 [SMILES: CC1CNCCN1c1cnn2cc(-c3cnn(C)c3)ccc12]

To a vial containing tert-butyl-3-methyl-4-(6-(1-methyl-1H-pyrazol-4-yl)pyrazolo[1,5-a]pyridin-3-yl)piperazine-1-carboxylate (93.0 mg, 0.24 mmol, as prepared in the previous step) was added 4M HCl in dioxane (3 mL). The reaction was stirred at rt for 2 h. The resulting mixture was concentrated under reduced pressure to afford 65.0 mg (61%) of 6-(1-methyl-1H-pyrazol-4-yl)-3-(2-methylpiperazin-1-yl)pyrazolo[1,5-a]pyridine as a light yellow oil. ¹H NMR (400 MHZ, DMSO-d₆) δ (ppm) 9.26 (m, 2H), 8.89 (s, 1H), 8.24 (s, 1H), 7.97 (d, J=9.7 Hz, 2H), 7.68 (d, J=9.1 Hz, 1H), 7.46 (d, J=9.2 Hz, 1H), 3.88-3.76 (m, 3H), 3.39-3.35 (m, 2H), 3.31 (d, J=11.8 Hz, 2H), 3.17 (d, J=11.8 Hz, 2H), 2.84 (d, J=11.3 Hz, 1H), 0.88 (d, J=6.2 Hz, 4H). MS (ESI) m/z [M+H]⁺ calcd. for C₁₆H₂₀N₆, 297.2; found, 297.1.

### Step 3. 3-(4-(5-benzylpyrimidin-2-yl)-2-methylpiperazin-1-yl)-6-(1-methyl-1H-pyrazol-4-yl)pyrazolo[1,5-a]pyridine (Compound 142)

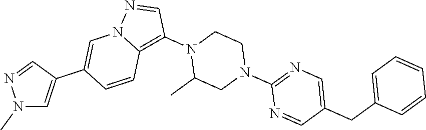 [SMILES: CC1CN(c2ncc(Cc3ccccc3)cn2)CCN1c1cnn2cc(-c3cnn(C)c3)ccc12]

To a solution of 6-(1-methyl-1H-pyrazol-4-yl)-3-(2-methylpiperazin-1-yl)pyrazolo[1,5-a]pyridine (65 mg, 0.22 mmol, as prepared in the previous step) dissolved in IPA (3 mL) was added 5-benzyl-2-chloropyrimidine (Compound S88) (44.9 mg, 0.22 mmol) and DIPEA (56.5 mg, 0.44 mmol). The reaction was stirred 130° C. for 12 h. The mixture was allowed to cool to rt. The residue was applied onto a silica gel column with EtOAc/PE (1/1). The crude product was purified by Prep-HPLC to afford 39.1 mg (38%) of 3-(4-(5-benzylpyrimidin-2-yl)-2-methylpiperazin-1-yl)-6-(1-methyl-1H-pyrazol-4-yl)pyrazolo[1,5-a]pyridine (Compound 142) as a yellow oil. ¹H NMR (400 MHZ, DMSO-d₆) δ (ppm) 8.84 (s, 1H), 8.31 (s, 2H), 8.22 (s, 1H), 7.97 (s, 1H), 7.92 (s, 1H), 7.62 (d, J=9.2 Hz, 1H), 7.37-7.19 (m, 6H), 4.36-4.35 (m, 2H), 3.87 (s, 3H), 3.80 (s, 2H), 3.44-3.43 (m, 1H), 3.12-3.05 (m, 3H), 3.00 (d, J=9.7 Hz, 1H), 0.86 (d, J=5.3 Hz, 3H). MS (ESI) m/z [M+H]⁺ calcd. for C₁₇H₂₈N₈, 465.2; found, 465.1; HPLC purity: 254 nm: 99.6%.

### Example 112: Synthesis of Exemplary Compound 143

### 3-(4-(5-benzylpyrimidin-2-yl)-3-methylpiperazin-1-yl)-6-(1-methyl-1H-pyrazol-4-yl)pyrazolo[1,5-a]pyridine (Compound 143)

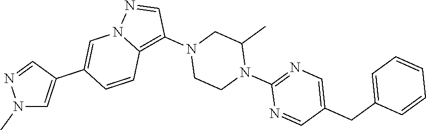 [SMILES: CC1CN(c2cnn3cc(-c4cnn(C)c4)ccc23)CCN1c1ncc(Cc2ccccc2)cn1]

### Step 1. Preparation of tert-butyl-2-methyl-4-(6-(1-methyl-1H-pyrazol-4-yl)pyrazolo[1,5-a]pyridin-3-yl)piperazine-1-carboxylate

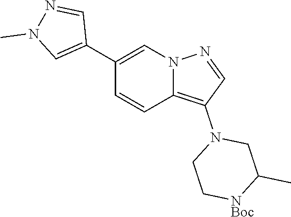

To a solution of 3-bromo-6-(1-methyl-1H-pyrazol-4-yl)pyrazolo[1,5-a]pyridine (Compound S9) (2.00 g, 7.22 mmol) dissolved in dioxane (10.0 mL) and tBuOH (10.0 mL) was added tert-butyl 2-methylpiperazine-1-carboxylate (1.54 g, 7.22 mmol), KOtBu (1.21 g, 10.80 mmol) and tBuXPhosPd G1 (744 mg, 1.08 mmol). The reaction was stirred at 90° C. for 16 h under nitrogen. The mixture was allowed to cool to rt, then diluted with water (20 mL). Then the solution was extracted with EtOAc (3×40 mL) then the organic layers combined and concentrated. The crude product was purified by Prep-HPLC to afford 1.10 g (38%) of tert-butyl-2-methyl-4-(6-(1-methyl-1H-pyrazol-4-yl)pyrazolo[1,5-a]pyridin-3-yl)piperazine-1-carboxylate as a light yellow oil. ¹H NMR (400 MHZ, DMSO-d₆) δ (ppm) 8.84 (s, 1H), 8.23 (s, 1H), 7.98 (s, 1H), 7.76 (s, 1H), 7.58 (d, J=9.2 Hz, 1H), 7.34 (d, J=9.3 Hz, 1H), 4.22 (m, 1H), 3.87 (s, 3H), 3.85-3.79 (m, 1H), 3.28-3.21 (m, 2H), 3.11 (d, J=11.5 Hz, 1H), 2.77 (d, J=3.8 Hz, 1H), 2.70-2.59 (m, 1H), 1.43 (s, 9H), 1.34 (d, J=6.7 Hz, 3H). MS (ESI) m/z [M+H]⁺ calcd. for C₂₁H₁₈N₆O₂, 397.2; found, 397.2.

### Step 2. Preparation of 6-(1-methyl-1H-pyrazol-4-yl)-3-(3-methylpiperazin-1-yl)pyrazolo[1,5-a]pyridine

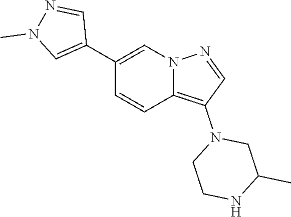

To a vial containing tert-butyl-2-methyl-4-(6-(1-methyl-1H-pyrazol-4-yl)pyrazolo[1,5-a]pyridin-3-yl)piperazine-1-carboxylate (1.00 g, 2.52 mmol, as prepared in the previous step) was added 4 M HCl in dioxane (10.0 mL). The reaction was stirred at rt for 2 h. The resulting mixture was concentrated under reduced pressure and diluted with water (20 mL). The mixture was basified to pH 10 with sat. aq. Na₂CO₃. The resulting mixture was extracted with DCM/MeOH (10/1) (3×30 mL) and concentrated under reduced pressure. The crude product was purified by Prep-HPLC to afford 350 mg (47%) of 6-(1-methyl-1H-pyrazol-4-yl)-3-(3-methylpiperazin-1-yl)pyrazolo[1,5-a]pyridine as a light yellow oil. ¹H NMR (400 MHZ, DMSO-d₆) δ (ppm) 8.79 (s, 1H), 8.22 (s, 1H), 7.96 (s, 1H), 7.71 (s, 1H), 7.61 (d, J=9.2 Hz, 1H), 7.28 (d, J=9.2 Hz, 1H), 3.87 (s, 3H), 3.16 (t, J=7.6 Hz, 2H), 2.94-2.90 (m, 2H), 2.66-2.57 (m, 2H), 2.28 (d, J=10.3 Hz, 1H), 2.11 (d, J=22.3 Hz, 1H), 1.00 (d, J=6.3 Hz, 3H). MS (ESI) m/z [M+H]⁺ calcd. for C₁₆H₂₀N₆, 297.2; found, 297.1.

### Step 3. 3-(4-(5-benzylpyrimidin-2-yl)-3-methylpiperazin-1-yl)-6-(1-methyl-1H-pyrazol-4-yl)pyrazolo[1,5-a]pyridine (Compound 143)

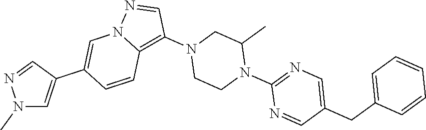 [SMILES: CC1CN(c2cnn3cc(-c4cnn(C)c4)ccc23)CCN1c1ncc(Cc2ccccc2)cn1]

To a solution of 6-(1-methyl-1H-pyrazol-4-yl)-3-(3-methylpiperazin-1-yl)pyrazolo[1,5-a]pyridine (310 mg, 1.05 mmol, as prepared in the previous step) dissolved in IPA (5 mL) was added 5-benzyl-2-chloropyrimidine (Compound S88) (214.9 mg, 1.05 mmol) and DIPEA (270 mg, 2.09 mmol). The resulting solution was stirred 130° C. for 10 days. The mixture was allowed to cool to rt. The resulting mixture was diluted with water (10 mL). The resulting solution was extracted with EtOAc (3×20 mL) then the organic layers were combined and concentrated. The residue was applied onto a silica gel column eluting with EtOAc/PE (1/1). The crude product was purified by Prep-HPLC to afford 91.4 mg (18%) of 3-(4-(5-benzylpyrimidin-2-yl)-3-methylpiperazin-1-yl)-6-(1-methyl-1H-pyrazol-4-yl)pyrazolo[1,5-a]pyridine (Compound 143) as a light yellow solid. ¹H NMR (400 MHZ, DMSO-d₆) δ (ppm) 8.84 (s, 1H), 8.32 (s, 2H), 8.23 (s, 1H), 7.98 (s, 1H), 7.77 (s, 1H), 7.63 (d, J=9.3 Hz, 1H), 7.36-7.20 (m, 6H), 4.88 (s, 1H), 4.49 (d, J=13.4 Hz, 1H), 3.88 (s, 3H), 3.80-3.76 (m, 2H), 3.37-3.36 (m, 1H), 3.27 (s, 1H), 3.23 (d, J=11.4 Hz, 1H), 2.86 (m, 1H), 2.72 (d, J=3.5 Hz, 1H), 1.36 (d, J=6.6 Hz, 3H). MS (ESI) m/z [M+H]⁺ calcd. for C₁₇H₂₈N₈, 465.2; found, 465.2; HPLC purity: 254 nm: 99.9%.

### Example 113: Synthesis of Exemplary Compound 144

### 1-(5-benzylpyrimidin-2-yl)-4-(6-(1-methyl-1H-pyrazol-4-yl)pyrazolo[1,5-a]pyridin-3-yl)piperazin-2-one (Compound 144)

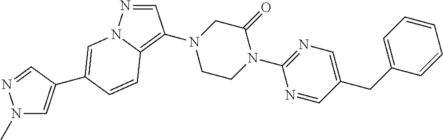 [SMILES: Cn1cc(-c2ccc3c(N4CCN(c5ncc(Cc6ccccc6)cn5)C(=O)C4)cnn3c2)cn1]

### Step 1. Preparation of ethyl N-(2-((tert-butoxycarbonyl)amino)ethyl)-N-(6-(1-methyl-1H-pyrazol-4-yl)pyrazolo[1,5-a]pyridin-3-yl)glycinate

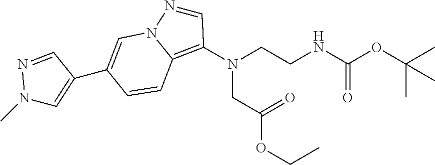 [SMILES: CCOC(=O)CN(CCNC(=O)OC(C)(C)C)c1cnn2cc(-c3cnn(C)c3)ccc12]

To a solution of tert-butyl (2-((6-(1-methyl-1H-pyrazol-4-yl)pyrazolo[1,5-a]pyridin-3-yl)amino)ethyl)carbamate (100 mg, 0.28 mmol, as prepared in Example 99, Step 1), ethyl iodoacetate (72.1 mg, 0.34 mmol) in DMF (5 mL) was added K₂CO₃ (116.3 mg, 0.84 mmol) at rt. The mixture was stirring at 90° C. for 12 h, then allowed to cool to rt. The resulting mixture was diluted with H₂O (15 mL) and extracted with EtOAc (3×50 mL). The combined organic layers dried over anhydrous Na₂SO₄. After filtration, the filtrate was concentrated under reduced pressure. The residue was purified by Prep-TLC (PE/EtOAc 1:5) to afford 40 mg (32%) of ethyl N-(2-((tert-butoxycarbonyl)amino)ethyl)-N-(6-(1-methyl-1H-pyrazol-4-yl)pyrazolo[1,5-a]pyridin-3-yl)glycinate as a yellow oil. ¹H NMR (400 MHZ, DMSO-d₆) δ (ppm) 9.06 (s, 1H), 8.52 (s, 1H), 8.36-8.27 (m, 2H), 8.10 (d, J=9.4 Hz, 1H), 7.77 (d, J=9.1 Hz, 1H), 6.48 (s, 1H), 4.66-4.54 (m, 2H), 4.46-4.36 (m, 5H), 3.90-3.79 (m, 2H), 3.71-3.60 (m, 2H), 1.86 (s, 9H), 1.73-1.63 (m, 3H); MS (ESI) m/z [M+H]⁺ calcd. for C₂₂H₃₀N₆O₄, 443.2; found, 443.3.

### Step 2. Preparation of ethyl N-(2-aminoethyl)-N-(6-(1-methyl-1H-pyrazol-4-yl)pyrazolo[1,5-a]pyridin-3-yl)glycinate hydrochloride

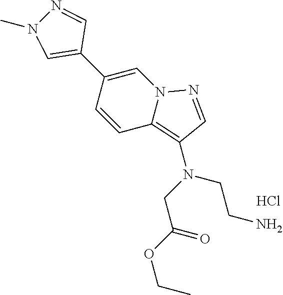 [SMILES: CCOC(=O)CN(CCN)c1cnn2cc(-c3cnn(C)c3)ccc12 ; Cl]

To a solution of 4M HCl in dioxane (5 mL) was added ethyl N-(2-((tert-butoxycarbonyl)amino)ethyl)-N-(6-(1-methyl-1H-pyrazol-4-yl)pyrazolo[1,5-a]pyridin-3-yl)glycinate (80 mg, 0.18 mmol, as prepared in the previous step) at rt. The mixture was stirred at rt for 2 h, then concentrated under reduced pressure to afford 30 mg (49%) of ethyl N-(2-aminoethyl)-N-(6-(1-methyl-1H-pyrazol-4-yl)pyrazolo[1,5-a]pyridin-3-yl)glycinate hydrochloride as a yellow oil. ¹H NMR (400 MHZ, DMSO-d₆) δ (ppm) 8.82 (s, 1H), 8.23 (s, 1H), 8.00-7.85 (m, 4H), 7.72 (dd, J=9.3, 1.0 Hz, 1H), 7.37 (dd, J=9.3, 1.5 Hz, 1H), 6.72 (s, 1H), 4.09-4.04 (m, 2H), 3.98 (s, 2H), 3.48 (t, J=6.3 Hz, 2H), 2.92-2.82 (m, 2H), 1.76 (s, 3H), 1.16 (t, J=7.1 Hz, 3H). MS (ESI) m/z [M+H]⁺ calcd. for C₁₇H₂₂N₆O₂, 343.2; found, 343.3.

### Step 3. Preparation of 4-(6-(1-methyl-1H-pyrazol-4-yl)pyrazolo[1,5-a]pyridin-3-yl)piperazin-2-one

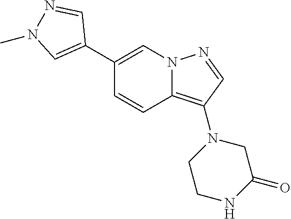 [SMILES: Cn1cc(-c2ccc3c(N4CCNC(=O)C4)cnn3c2)cn1]

To a solution of ethyl N-(2-aminoethyl)-N-(6-(1-methyl-1H-pyrazol-4-yl)pyrazolo[1,5-a]pyridin-3-yl)glycinate hydrochloride (100 mg, 0.29 mmol, as prepared in the previous step) in IPA (5 mL) was added DIPEA (113.2 mg, 0.88 mmol) at rt. The reaction was stirred at 90° C. for 12 h, then allowed to cool to rt. The resulting mixture was diluted with H₂O (15 mL) and was extracted with EtOAc (3×50 mL). The combined organic layers were dried over anhydrous Na₂SO₄. After filtration, the filtrate was concentrated under reduced pressure. The residue was purified by Prep-TLC (PE/EtOAc 1:5) to afford 15 mg (17%) of 4-(6-(1-methyl-1H-pyrazol-4-yl)pyrazolo[1,5-a]pyridin-3-yl)piperazin-2-one as a yellow solid. ¹H NMR (400 MHZ, DMSO-d₆) δ (ppm) 8.84 (s, 1H), 8.23 (s, 1H), 8.03-7.91 (m, 2H), 7.81 (s, 1H), 7.69 (d, J=9.3 Hz, 1H), 7.35 (d, J=9.2 Hz, 1H), 3.87 (s, 3H), 3.58 (s, 2H), 3.23-3.14 (m, 4H). MS (ESI) m/z [M+H]⁺ calcd. for C₁₅H₁₆N₆O, 297.2; found, 297.3.

### Step 4. 1-(5-benzylpyrimidin-2-yl)-4-(6-(1-methyl-1H-pyrazol-4-yl)pyrazolo[1,5-a]pyridin-3-yl)piperazin-2-one (Compound 144)

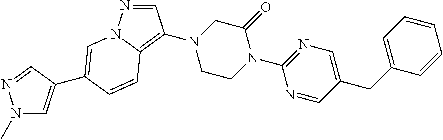 [SMILES: Cn1cc(-c2ccc3c(N4CCN(c5ncc(Cc6ccccc6)cn5)C(=O)C4)cnn3c2)cn1]

To a solution of 5-benzyl-2-chloropyrimidine (Compound S88) (70 mg, 0.34 mmol), 4-(6-(1-methyl-1H-pyrazol-4-yl)pyrazolo[1,5-a]pyridin-3-yl)piperazin-2-one (101.4 mg, 0.34 mmol, as prepared in the previous step), Xantphos (19.8 mg, 0.03 mmol), and K₃PO₄ (217.8 mg, 1.03 mmol) in dioxane (5 mL) was added Pd₂(dba)₃ (31.3 mg, 0.03 mmol) at rt. The mixture was stirred at 90° C. for 6 h, then allowed to cool to rt. The resulting mixture was diluted with H₂O (15 mL) and was extracted with EtOAc (3×50 mL). The combined organic layers dried over anhydrous Na₂SO₄. After filtration, the filtrate was concentrated under reduced pressure. The crude product was purified by Prep-HPLC to afford 19.4 mg (12%) of 1-(5-benzylpyrimidin-2-yl)-4-(6-(1-methyl-1H-pyrazol-4-yl)pyrazolo[1,5-a]pyridin-3-yl)piperazin-2-one (Compound 144) as a yellow solid. ¹H NMR (400 MHZ, DMSO-d₆) δ (ppm) 8.87 (s, 1H), 8.77 (s, 2H), 8.25 (s, 1H), 7.99 (s, 1H), 7.88 (s, 1H), 7.76 (d, J=9.2 Hz, 1H), 7.41-7.30 (m, 5H), 7.30-7.20 (m, 1H), 4.03 (s, 2H), 4.00-3.93 (m, 2H), 3.90-3.83 (m, 5H), 3.45 (t, J=5.4 Hz, 2H). MS (ESI) m/z [M+H]⁺ calcd. for C₂₆H₂₄N₈O, 465.2; found, 465.3; HPLC purity: 254 nm: 96.3%.

### Example 114: Synthesis of Exemplary Compound 145

### benzyl 1-(6-(1-methyl-1H-pyrazol-4-yl)pyrazolo[1,5-a]pyridin-3-yl)piperidine-4-carboxylate (Compound 145)

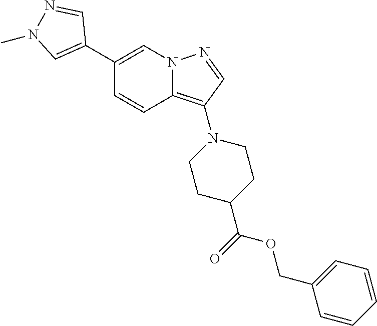 [SMILES: Cn1cc(-c2ccc3c(N4CCC(C(=O)OCc5ccccc5)CC4)cnn3c2)cn1]

### Step 1. Preparation of methyl 1-(6-(1-methyl-1H-pyrazol-4-yl)pyrazolo[1,5-a]pyridin-3-yl)piperidine-4-carboxylate

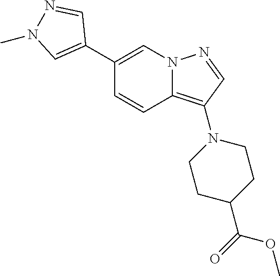

To a stirred solution of 3-bromo-6-(1-methyl-1H-pyrazol-4-yl)pyrazolo[1,5-a]pyridine (Compound S9) (3 g, 10.82 mmol) and methyl piperidine-4-carboxylate (7.75 g, 54.12 mmol) in tBuOH (40 mL) and dioxane (40 mL) were added KOtBu (1.82 g, 16.24 mmol) and tBuXPhos Pd G₁ (1.49 g, 2.16 mmol) at rt. The reaction was stirred for 16 h at 60° C. under nitrogen. The resulting mixture was filtered, the filter cake was washed with DCM (3×80 mL), and the filtrate was concentrated under reduced pressure. The residue was purified by reversed-phase flash chromatography with C₁₈ silica gel column eluting with 20% to 70% ACN in water (10 mM NH₄HCO₃), to afford 1.6 g (44%) of methyl 1-(6-(1-methyl-1H-pyrazol-4-yl)pyrazolo[1,5-a]pyridin-3-yl)piperidine-4-carboxylate as a yellow solid. ¹H NMR (400 MHZ, DMSO-d₆) δ (ppm) 8.80 (s, 1H), 8.22 (s, 1H), 7.97 (s, 1H), 7.73 (s, 1H), 7.61 (d, J=9.2 Hz, 1H), 7.30 (dd, J=9.2, 1.5 Hz, 1H), 3.87 (s, 3H), 3.65 (s, 3H), 3.32-3.26 (m, 2H), 2.79-2.65 (m, 2H), 2.49-2.44 (m, 1H), 1.99-1.91 (m, 2H), 1.87-1.74 (m, 2H). MS (ESI) m/z [M+H]⁺ calcd. for C₁₈H₂₁N₅O₂, 340.2; found, 340.3.

### Step 2. Preparation of 1-(6-(1-methyl-1H-pyrazol-4-yl)pyrazolo[1,5-a]pyridin-3-yl)piperidine-4-carboxylic acid

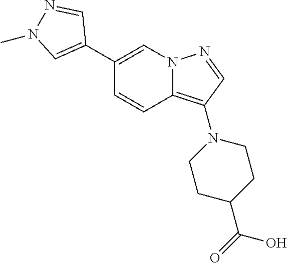 [SMILES: Cn1cc(-c2ccc3c(N4CCC(C(=O)O)CC4)cnn3c2)cn1]

To a stirred solution of methyl 1-(6-(1-methyl-1H-pyrazol-4-yl)pyrazolo[1,5-a]pyridin-3-yl)piperidine-4-carboxylate (600 mg, 1.77 mmol, as prepared in the previous step) in MeOH (5 mL) was added 1M NaOH (10 mL) at rt. The reaction was stirred for 1 h then acidified to pH 6 with conc. HCl. The resulting mixture was extracted with DCM (3×100 mL). The combined organic layers were dried over anhydrous Na₂SO₄. After filtration, the filtrate was concentrated under reduced pressure to afford 460 mg (80%) of 1-(6-(1-methyl-1H-pyrazol-4-yl)pyrazolo[1,5-a]pyridin-3-yl)piperidine-4-carboxylic acid as a yellow solid. ¹H NMR (400 MHZ, DMSO-d₆) δ 12.21 (s, 1H), 8.80 (s, 1H), 8.22 (s, 1H), 7.96 (s, 1H), 7.73 (s, 1H), 7.61 (d, J=9.2 Hz, 1H), 7.37-7.25 (m, 1H), 3.87 (s, 3H), 3.32-3.23 (m, 2H), 2.83-2.68 (m, 2H), 2.44-2.30 (m, 1H), 2.03-1.69 (m, 4H). MS (ESI) m/z [M+H]⁺ calcd. for C₁₇H₁₉N₅O₂, 326.2; found, 326.3.

### Step 3. Preparation of benzyl 1-(6-(1-methyl-1H-pyrazol-4-yl)pyrazolo[1,5-a]pyridin-3-yl)piperidine-4-carboxylate (Compound 145)

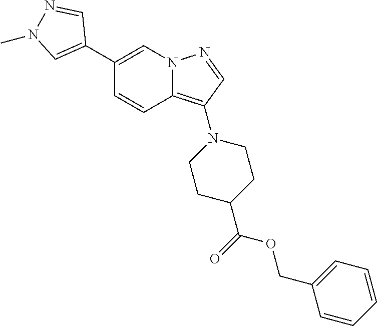 [SMILES: Cn1cc(-c2ccc3c(N4CCC(C(=O)OCc5ccccc5)CC4)cnn3c2)cn1]

To a stirred solution of 1-(6-(1-methyl-1H-pyrazol-4-yl)pyrazolo[1,5-a]pyridin-3-yl)piperidine-4-carboxylic acid (400 mg, 1.23 mmol, as prepared in the previous step) and K₂CO₃ (339.8 mg, 2.46 mmol) in DMF (7 mL) was added benzyl bromide (210.3 mg, 1.23 mmol) at 0° C. The reaction was stirred for 2 h at rt. The resulting mixture was filtered, the filter cake was washed with DCM (3×70 mL). The filtrate was concentrated under reduced pressure. The crude product was purified by Prep-HPLC to afford 168.6 mg (33%) of benzyl 1-(6-(1-methyl-1H-pyrazol-4-yl)pyrazolo[1,5-a]pyridin-3-yl)piperidine-4-carboxylate (Compound 145) as a light green solid. ¹H NMR (400 MHZ, DMSO-d₆) δ (ppm) 8.80 (s, 1H), 8.22 (s, 1H), 7.97 (s, 1H), 7.72 (s, 1H), 7.62 (d, J=9.2 Hz, 1H), 7.46-7.16 (m, 6H), 5.15 (s, 2H), 3.87 (s, 3H), 3.32-3.24 (m, 2H), 2.81-2.64 (m, 2H), 2.61-2.54 (m, 1H), 2.02-1.93 (m, 2H), 1.90-1.75 (m, 2H). MS (ESI) m/z [M+H]⁺ calcd. for C₂₄H₂₅N₅O₂, 416.2; found, 416.15; HPLC purity: 254 nm: 99.9%.

### Example 115: Synthesis of Exemplary Compound 146

### N-benzyl-1-(6-(1-methyl-1H-pyrazol-4-yl)pyrazolo[1,5-a]pyridin-3-yl)piperidine-4-carboxamide (Compound 146)

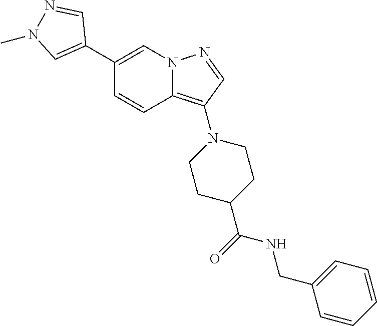 [SMILES: Cn1cc(-c2ccc3c(N4CCC(C(=O)NCc5ccccc5)CC4)cnn3c2)cn1]

### Step 1. Preparation of tert-butyl 4-(benzylcarbamoyl)piperidine-1-carboxylate

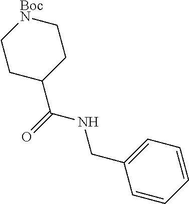 [SMILES: CC(C)(C)OC(=O)N1CCC(C(=O)NCc2ccccc2)CC1]

To a stirred solution of 1-(tert-butoxycarbonyl)piperidine-4-carboxylic acid (2 g, 8.72 mmol) and benzylamine (935 mg, 8.72 mmol) in DCM (40 mL) were added HATU (4.98 g, 13.08 mmol) and DIPEA (3.38 g, 26.17 mmol) at 0° C. The reaction was stirred for 1 h at rt. The resulting mixture was filtered, and the filter cake was washed with DCM (3×100 mL). The filtrate was concentrated under reduced pressure. The residue was purified by reverse flash chromatography with the following conditions C₁₈ silica gel column eluting with 10% to 50% ACN in water (10 mM NH₄HCO₃) to afford 2.7 g (97%) of tert-butyl 4-(benzylcarbamoyl)piperidine-1-carboxylate as a white solid. ¹H NMR (400 MHZ, DMSO-d₆) δ (ppm) 8.37 (s, 1H), 7.37-7.17 (m, 5H), 4.26 (d, J=5.9 Hz, 2H), 3.95 (d, J=12.7 Hz, 2H), 2.73 (s, 2H), 2.36 (t, J=9.8 Hz, 1H), 1.69 (d, J=13.0 Hz, 2H), 1.50-1.43 (m, 2H), 1.40 (s, 9H). MS (ESI) m/z [M+H]⁺ calcd. for C₁₈H₂₆N₂O₃, 319.2; found, 319.3.

### Step 2. Preparation of N-benzylpiperidine-4-carboxamide

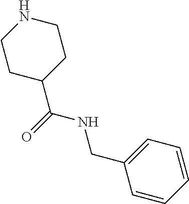 [SMILES: O=C(NCc1ccccc1)C1CCNCC1]

To a stirred solution of tert-butyl 4-(benzylcarbamoyl)piperidine-1-carboxylate (1.5 g, 4.71 mmol, as prepared in the previous step) in DCM (10 mL) was added 4M HCl in dioxane (11.8 mL) at 0° C. The reaction was stirred for 1 h at rt. The resulting mixture was concentrated under reduced pressure. The residue was dissolved in water (50 mL) and basified to pH 10 with sat. aq. Na₂CO₃. The resulting mixture was extracted with DCM (3×100 mL). The combined organic layers were dried over anhydrous Na₂SO₄. After filtration, the filtrate was concentrated under reduced pressure to afford 1 g (97%) of N-benzylpiperidine-4-carboxamide as a white solid. ¹H NMR (400 MHZ, DMSO-d₆) δ (ppm) 8.27 (s, 1H), 7.37-7.16 (m, 5H), 4.25 (d, J=4.4 Hz, 2H), 2.94 (d, J=11.8 Hz, 2H), 2.44 (t, J=12.4 Hz, 3H), 2.25 (t, J=11.1 Hz, 1H), 1.60 (d, J=11.9 Hz, 2H), 1.54-1.37 (m, 2H). MS (ESI) m/z [M+H]⁺ calcd. for C₁₃H₁₈N₂O, 219.1; found, 219.3.

Step 3. Preparation of N-benzyl-1-(6-(1-methyl-1H-pyrazol-4-yl)pyrazolo[1,5-a]pyridin-3-yl)piperidine-4-carboxamide (Compound 146)

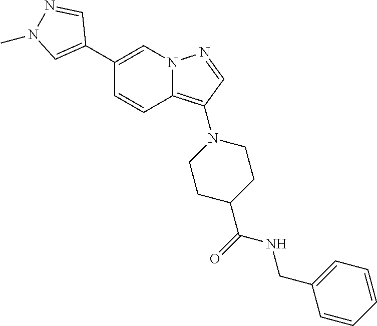 [SMILES: Cn1cc(-c2ccc3c(N4CCC(C(=O)NCc5ccccc5)CC4)cnn3c2)cn1]

To a stirred solution of N-benzylpiperidine-4-carboxamide (993 mg, 4.55 mmol, as prepared in the previous step) and 3-bromo-6-(1-methyl-1H-pyrazol-4-yl)pyrazolo[1,5-a]pyridine (Compound S9) (252 mg, 0.91 mmol) in tBuOH (8 mL) and dioxane (8 mL) were added KOtBu (153 mg, 1.36 mmol) and tBuXPhos Pd G1 (93.7 mg, 0.14 mmol) at rt. The reaction was stirred overnight at 60° C. under nitrogen. The resulting mixture was filtered, the filter cake was washed with DCM (3×100 mL). The filtrate was concentrated under reduced pressure. The residue was purified by silica gel column chromatography eluting with DCM/MeOH (15:1) and then purified by Prep-HPLC to afford 33.8 mg (9%) of N-benzyl-1-(6-(1-methyl-1H-pyrazol-4-yl)pyrazolo[1,5-a]pyridin-3-yl)piperidine-4-carboxamide (Compound 146) as a light yellow solid. ¹H NMR (400 MHZ, DMSO-d₆) δ (ppm) 8.80 (s, 1H), 8.37 (t, J=6.0 Hz, 1H), 8.22 (s, 1H), 7.97 (s, 1H), 7.74 (s, 1H), 7.62 (d, J=9.2 Hz, 1H), 7.37-7.21 (m, 6H), 4.30 (d, J=5.9 Hz, 2H), 3.87 (s, 3H), 3.40-3.34 (m, 2H), 2.72-2.61 (m, 2H), 2.39-2.26 (m, 1H), 1.93-1.77 (m, 4H). MS (ESI) m/z [M+H]⁺ calcd. for C₂₄H₂₆N₆O, 415.2; found, 415.15; HPLC Purity: 254 nm: 99.5%.

Using the procedures described in Example 115, Compound 146 and reagents, starting materials, and conditions known to those skilled in the art were used to prepare the compounds shown in Table R.

TABLE R

| Compound | Name | Characterization |
|---|---|---|
| Compound | N-benzyl-N-methyl-1-(6-(1-methyl-1H- | ¹H NMR (400 MHz, DMSO- |
| 147 | pyrazol-4-yl)pyrazolo[1,5-a]pyridin-3- | d₆) δ (ppm) 8.80 (d, J = 7.1 |
| | yl)piperidine-4-carboxamide | Hz, 1H), 8.22 (d, J = 3.8 Hz, |
| | | 1H), 7.97 (d, J = 4.2 Hz, 1H), |
| | | 7.72 (d, J = 18.4 Hz, 1H), 7.62 |
| | | (t, J = 9.9 Hz, 1H), 7.50-7.11 |
| | | (m, 6H), 4.68 (s, 1H), 4.54 (s, |
| | | 1H), 3.87 (s, 3H), 3.42-3.35 |
| | | (m, 2H), 3.01 (s, 2H), 2.90- |
| | | 2.62 (m, 4H), 2.07-1.51 (m, |
| | | 4H). MS (ESI) m/z [M + H]⁺ |
| | | calcd. for C₂₅H₂₈N₆O, 429.2; |
| | | found, 429.20; HPLC purity: |
| | | 254 nm: 97.3%. |

### Example 116: Synthesis of Exemplary Compound 148

### 3-(4-(5-benzylpyrimidin-2-yl)piperidin-1-yl)-6-(1-methyl-1H-pyrazol-4-yl)pyrazolo[1,5-a]pyridine (Compound 148)

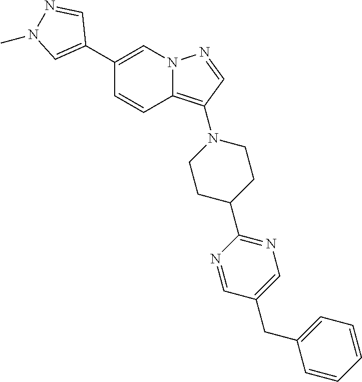 [SMILES: Cn1cc(-c2ccc3c(N4CCC(c5ncc(Cc6ccccc6)cn5)CC4)cnn3c2)cn1]

### Step 1. Preparation of tert-butyl 4-(5-benzylpyrimidin-2-yl)-3,6-dihydropyridine-1 (2H)-carboxylate

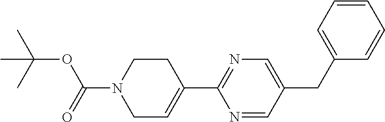 [SMILES: CC(C)(C)OC(=O)N1CC=C(c2ncc(Cc3ccccc3)cn2)CC1]

To a solution of tert-butyl 4-(4,4,5,5-tetramethyl-1,3,2-dioxaborolan-2-yl)-3,6-dihydro-2H-pyridine-1-carboxylate (2.00 g, 6.47 mmol) and 5-benzyl-2-chloropyrimidine (Compound S88) (1.19 g, 5.82 mmol) in dioxane (10.0 mL) and H₂O (1.0 mL) was added Pd(dppf)Cl₂·DCM (0.79 g, 0.97 mmol) and Cs₂CO₃ (4.21 g, 12.9 mmol) under nitrogen. The reaction was stirred for 3 h at 90° C. under nitrogen. The resulting mixture was filtered, and the filter cake was washed with EtOAc (2×5 mL). The filtrate was concentrated under reduced pressure. The residue was purified by silica gel column chromatography eluting with PE/EtOAc (9:1) to afford 2.06 g (82%) of tert-butyl 4-(5-benzylpyrimidin-2-yl)-3,6-dihydropyridine-1 (2H)-carboxylate as a white solid. ¹H NMR (400 MHZ, DMSO-d₆) δ (ppm) 8.69 (s, 2H), 7.36-7.17 (m, 5H), 7.11 (s, 1H), 4.07 (s, 2H), 3.98 (s, 2H), 3.52 (t, J=6.0 Hz, 2H), 2.63-2.54 (m, 2H), 1.43 (s, 9H). MS (ESI) m/z [M+H]⁺ calcd. For C₂₁H₂₅N₃O₂, 352.2; found, 352.3.

### Step 2. Preparation of 5-benzyl-2-(1,2,3,6-tetrahydropyridin-4-yl)pyrimidine

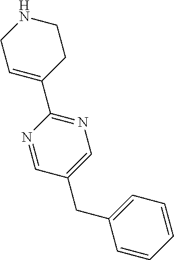 [SMILES: C1=C(c2ncc(Cc3ccccc3)cn2)CCNC1]

To a solution of tert-butyl 4-(5-benzylpyrimidin-2-yl)-3,6-dihydropyridine-1 (2H)-carboxylate (1.00 g, 2.85 mmol, as prepared in the previous step) in DCM (9.0 mL) was added TFA (3.0 mL) at 0° C. The reaction was stirred for 3 h at rt. The mixture was neutralized to pH 8 with sat. aq. Na₂CO₃. The resulting mixture was extracted with DCM (2×60 mL). The combined organic layers were dried over anhydrous Na₂SO₄. After filtration, the filtrate was concentrated under reduced pressure to afford 0.91 g of 5-benzyl-2-(1,2,3,6-tetrahydropyridin-4-yl)pyrimidine as a yellow solid. ¹H NMR (400 MHZ, DMSO-d₆) δ (ppm) 8.71 (s, 2H), 7.34-7.18 (m, 5H), 7.16-7.09 (m, 1H), 3.99 (s, 2H), 3.73-3.67 (m, 2H), 3.19-3.13 (m, 2H), 2.70-2.61 (m, 2H). MS (ESI) m/z [M+H]⁺ calcd. for C₁₆H₁₇N₃, 252.1; found, 252.2.

### Step 3. Preparation of 5-benzyl-2-(piperidin-4-yl)pyrimidine

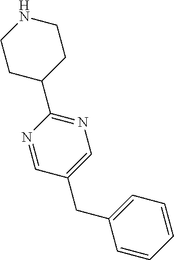 [SMILES: c1ccc(Cc2cnc(C3CCNCC3)nc2)cc1]

To a solution of 5-benzyl-2-(1,2,3,6-tetrahydropyridin-4-yl)pyrimidine (0.90 g, 3.58 mmol, as prepared in the previous step) in MeOH (10.0 mL) was added Pd/C (191 mg, 1.79 mmol). The reaction was stirred for 16 h at rt under hydrogen atmosphere. The resulting mixture was filtered, the filter cake was washed with MeOH (2×5 mL). The filtrate was concentrated under reduced pressure. The residue was purified by reversed-phase flash chromatography with C₁₈ silica gel column eluting with 10% to 50% ACN in water (10 mM NH₄HCO₃) to afford 440 mg (44%) of 5-benzyl-2-(piperidin-4-yl)pyrimidine as a yellow oil. ¹H NMR (400 MHZ, DMSO-d₆) δ (ppm) 8.64 (s, 2H), 7.35-7.17 (m, 5H), 3.94 (s, 2H), 3.04-2.94 (m, 2H), 2.89-2.77 (m, 1H), 2.62-2.51 (m, 2H), 1.83-1.75 m, 2H), 1.69-1.54 (m, 2H). MS (ESI) m/z [M+H]⁺ calcd. for C₁₆H₁₉N₃, 254.2; found, 254.3.

### Step 4. Preparation of 3-(4-(5-benzylpyrimidin-2-yl)piperidin-1-yl)-6-(1-methyl-1H-pyrazol-4-yl)pyrazolo[1,5-a]pyridine (Compound 148)

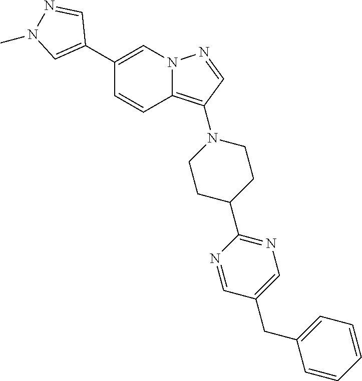 [SMILES: Cn1cc(-c2ccc3c(N4CCC(c5ncc(Cc6ccccc6)cn5)CC4)cnn3c2)cn1]

To a solution of 5-benzyl-2-(piperidin-4-yl)pyrimidine (420 mg, 1.66 mmol, as prepared in the previous step) and 3-bromo-6-(1-methyl-1H-pyrazol-4-yl)pyrazolo[1,5-a]pyridine (Compound S9) (413 mg, 1.49 mmol) in tBuOH (4.0 mL) and dioxane (1.0 mL) was added with KOtBu (279 mg, 2.49 mmol) and tBuXPhos Pd G1 (171 mg, 0.25 mmol) under nitrogen. The reaction was stirred for 40 h at 90° C. under nitrogen. The resulting mixture was concentrated under reduced pressure. The residue was purified by silica gel column chromatography eluting with PE/EtOAc (1:9). The crude product (240 mg) was purified by Prep-HPLC to afford 65.4 mg (9%) of 3-(4-(5-benzylpyrimidin-2-yl)piperidin-1-yl)-6-(1-methyl-1H-pyrazol-4-yl)pyrazolo[1,5-a]pyridine (Compound 148) as a yellow solid. ¹H NMR (400 MHZ, DMSO-d₆) δ (ppm) 8.80 (s, 1H), 8.68 (s, 2H), 8.22 (s, 1H), 7.96 (s, 1H), 7.75 (s, 1H), 7.63 (d, J=9.2 Hz, 1H), 7.37-7.26 (m, 5H), 7.26-7.14 (m, 1H), 3.97 (s, 2H), 3.87 (s, 3H), 3.46-3.37 (m, 2H), 2.98-2.87 (m, 1H), 2.87-2.76 (m, 2H), 2.10-1.96 (m, 4H). MS (ESI) m/z [M+H]⁺ calcd. for C₂₇H₂₇N₇, 450.2; found, 450.2; HPLC purity: 254 nm: 97.1%.

### Example 117: Synthesis of Exemplary Compound 149

### 5-benzyl-N-(1-(6-(1-methyl-1H-pyrazol-4-yl)pyrazolo[1,5-a]pyridin-3-yl) azetidin-3-yl)pyrimidin-2-amine (Compound 149)

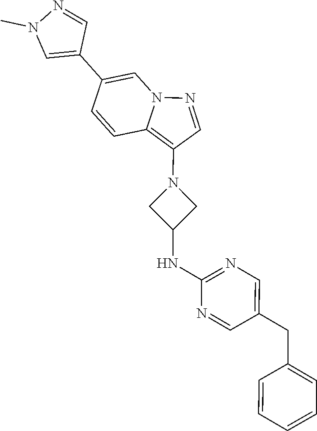 [SMILES: Cn1cc(-c2ccc3c(N4CC(Nc5ncc(Cc6ccccc6)cn5)C4)cnn3c2)cn1]

### Step 1. Preparation of tert-butyl 3-((5-benzylpyrimidin-2-yl)amino) azetidine-1-carboxylate

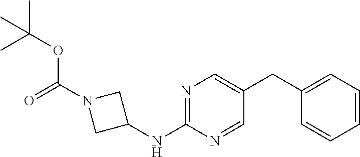 [SMILES: CC(C)(C)OC(=O)N1CC(Nc2ncc(Cc3ccccc3)cn2)C1]

To a solution of 5-benzyl-2-chloropyrimidine (Compound S88) (2.0 g, 9.77 mmol) dissolved in IPA (50 mL) was added tert-butyl 3-aminoazetidine-1-carboxylate (1.85 g, 10.75 mmol) and DIPEA (3.79 g, 29.32 mmol). The reaction was stirred for 16 h at 110° C. under nitrogen. The mixture was allowed to cool to rt. The resulting mixture was diluted with water (100 mL) and extracted with EtOAc (3×200 mL). The combined organic layers were washed with brine (2×100 mL) and dried over anhydrous Na₂SO₄. After filtration, the filtrate was concentrated under reduced pressure. The residue was purified by silica gel column chromatography eluting with PE/EtOAc (3:2) to afford 1.3 g (39%) of tert-butyl 3-((5-benzylpyrimidin-2-yl)amino)azetidine-1-carboxylate as a white solid. ¹H NMR (400 MHZ, DMSO-d₆) δ (ppm) 8.21 (s, 2H), 7.68 (d, J=6.4 Hz, 1H), 7.33-7.15 (m, 5H), 4.55-4.42 (m, 1H), 4.14-4.03 (m, 2H), 3.78-3.72 (m, 4H) 1.38 (s, 9H). MS (ESI) m/z [M+H]⁺ calcd. for C₁₉H₂₄N₄O₂, 341.2; found, 341.2.

### Step 2. Preparation of N-(azetidin-3-yl)-5-benzylpyrimidin-2-amine

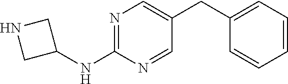 [SMILES: c1ccc(Cc2cnc(NC3CNC3)nc2)cc1]

To a solution of tert-butyl 3-((5-benzylpyrimidin-2-yl)amino) azetidine-1-carboxylate (800 mg, 2.35 mmol, as prepared in the previous step) dissolved in DCM (9 mL) was added 4M HCl in dioxane (3 mL). The resulting solution was stirred for 1 h at 0° C. under nitrogen atmosphere. The resulting mixture was concentrated under reduced pressure to afford 260 mg (44%) of N-(azetidin-3-yl)-5-benzylpyrimidin-2-amine as a yellow solid. ¹H NMR (400 MHZ, DMSO-d₆) δ (ppm) 8.35 (s, 2H), 8.24 (s, 2H), 7.77 (d, J=6.4 Hz, 1H), 7.32-7.21 (m, 5H), 4.55-4.51 (m, 1H), 4.07-3.99 (m, 2H), 3.88-3.81 (m, 2H), 3.77 (s, 2H). MS (ESI) m/z [M+H]⁺ calcd. for C₁₄H₁₆N₄, 241.1; found, 241.1.

### Step 3. Preparation of 5-benzyl-N-(1-(6-(1-methyl-1H-pyrazol-4-yl)pyrazolo[1,5-a]pyridin-3-yl) azetidin-3-yl)pyrimidin-2-amine (Compound 149)

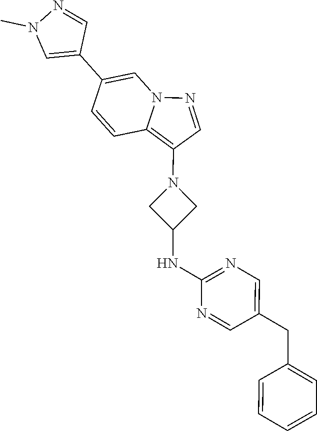 [SMILES: Cn1cc(-c2ccc3c(N4CC(Nc5ncc(Cc6ccccc6)cn5)C4)cnn3c2)cn1]

To a solution of N-(azetidin-3-yl)-5-benzylpyrimidin-2-amine (150 mg, 0.62 mmol, as prepared in the previous step) dissolved in dioxane (6 mL) was added 3-bromo-6-(1-methyl-1H-pyrazol-4-yl)pyrazolo[1,5-a]pyridine (Compound S9) (190.3 mg, 0.69 mmol), tBuXPhos Pd G1 (72.9 mg, 0.06 mmol) and KOtBu (140.1 mg, 1.25 mmol). The reaction was stirred for 16 h at 90° C. under nitrogen then allowed to cool to rt. The resulting mixture was diluted with water (50 mL) and extracted with EtOAc (3×50 mL). The combined organic layers were washed with brine (3×50 mL) and dried over anhydrous Na₂SO₄. After filtration, the filtrate was concentrated under reduced pressure. The crude product was purified by Prep-HPLC to afford 12.2 mg (4%) of 5-benzyl-N-(1-(6-(1-methyl-1H-pyrazol-4-yl)pyrazolo[1,5-a]pyridin-3-yl) azetidin-3-yl)pyrimidin-2-amine (Compound 149) as a light yellow solid. ¹H NMR (400 MHz, DMSO-d₆) δ (ppm) 8.72 (s, 1H), 8.22 (s, 2H), 8.19 (s, 1H), 7.94 (s, 1H), 7.68 (d, J=6.8 Hz, 1H), 7.54-7.47 (m, 2H), 7.36-7.13 (m, 6H), 4.79-4.66 (m, 1H), 4.24 (t, J=7 Hz, 2H), 3.86 (s, 3H), 3.77 (s, 2H), 3.67 (t, J=6.6 Hz, 2H). MS (ESI) m/z [M+H]⁺ calcd. for C₂₅H₂₄N₈, 437.2; found, 437.3; HPLC purity: 254 nm: 99.3%.

### Example 118: Synthesis of Exemplary Compound 150

### 3-(4-(4-benzylphenyl)piperazin-1-yl)-6-(1-methyl-1H-pyrazol-4-yl)pyrazolo[1,5-a]pyridine (Compound 150)

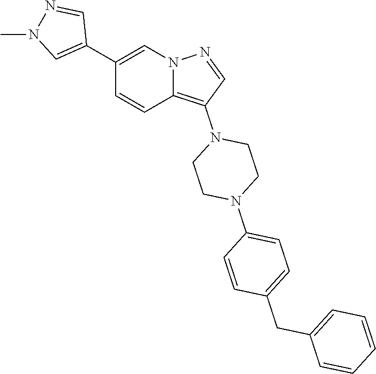 [SMILES: Cn1cc(-c2ccc3c(N4CCN(c5ccc(Cc6ccccc6)cc5)CC4)cnn3c2)cn1]

To a solution of 6-(1-methyl-1H-pyrazol-4-yl)-3-piperazin-1-ylpyrazolo[1,5-a]pyridine (Compound 12) (150 mg, 0.53 mmol) and 1-benzyl-4-bromobenzene (131 mg, 0.53 mmol) in tBuOH (4.0 mL) and dioxane (2.0 mL) were added KOtBu (89.4 mg, 0.79 mmol) and [2-(2-aminoethyl)phenyl]palladium; di-tert-butyl([2-chloro-6-[3-methyl-2,4,6-tris(propan-2-yl)phenyl]phenyl])phosphane (54.7 mg, 0.08 mmol) under nitrogen. The reaction was stirred for 2 h at 90° C. under nitrogen. The resulting mixture was concentrated under reduced pressure. The residue was purified by Prep-TLC (DCM/MeOH 20:1). The crude product (200 mg) was purified by Prep-HPLC to afford 87.7 mg (36%) of 3-(4-(4-benzylphenyl)piperazin-1-yl)-6-(1-methyl-1H-pyrazol-4-yl)pyrazolo[1,5-a]pyridine (Compound 150) as a white solid. ¹H NMR (400 MHZ, DMSO-d₆) δ (ppm) 8.84 (s, 1H), 8.23 (s, 1H), 7.98 (s, 1H), 7.80 (s, 1H), 7.68 (d, J=9.2 Hz, 1H), 7.36-7.24 (m, 3H), 7.24-7.14 (m, 3H), 7.13-7.06 (m, 2H), 6.97-6.88 (m, 2H), 3.93-3.78 (m, 5H), 3.31-3.23 (m, 4H), 3.18-3.10 (m, 4H). MS (ESI) m/z [M+H]⁺ calcd. for C₂₈H₂₈N₆, 449.2; found, 449.2; HPLC purity: 254 nm: 99.1%.

### Example 119: Synthesis of Exemplary Compound 151

### 3-(4-(6-benzylpyridin-3-yl)piperazin-1-yl)-6-(1-methyl-1H-pyrazol-4-yl)pyrazolo[1,5-a]pyridine (Compound 151)

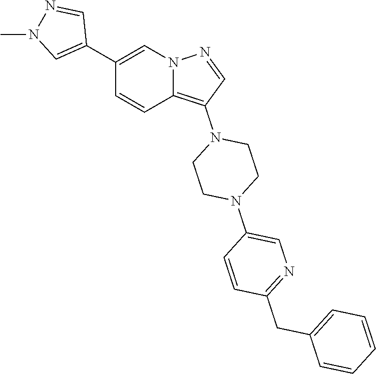 [SMILES: Cn1cc(-c2ccc3c(N4CCN(c5ccc(Cc6ccccc6)nc5)CC4)cnn3c2)cn1]

To a solution of (5-{4-[6-(1-methylpyrazol-4-yl)pyrazolo[1,5-a]pyridin-3-yl]piperazin-1-yl}pyridin-2-yl)(phenyl)methanol (Compound 241) (140 mg, 0.30 mmol) in DCM (15 mL) was added TFA (1.37 g, 12.04 mmol) and Et₃SiH (699.3 mg, 6.02 mmol). After stirring overnight at rt under nitrogen, the mixture was basified to pH 8 with sat. aq. NaHCO₃. The aqueous layer was extracted with DCM (3×10 mL). The resulting mixture was concentrated under vacuum and purified by Prep-HPLC to afford 26.6 mg (19%) of 3-(4-(6-benzylpyridin-3-yl)piperazin-1-yl)-6-(1-methyl-1H-pyrazol-4-yl)pyrazolo[1,5-a]pyridine (Compound 151) as a yellow solid. ¹H NMR (400 MHZ, DMSO-d₆) δ (ppm) 8.84 (s, 1H), 8.29-8.21 (m, 2H), 7.98 (d, J=0.8 Hz, 1H), 7.80 (s, 1H), 7.71-7.64 (m, 1H), 7.39-7.06 (m, 8H), 3.98 (s, 2H), 3.87 (s, 3H), 3.40-3.30 (m, 4H), 3.14 (t, J=5.1 Hz, 4H). MS (ESI) m/z [M+H]⁺ calcd. for C₂₇H₂₇N₇: 450.2, found: 450.2. HPLC purity: 254 nm: 97.7%.

Using the procedures described in Example 119, Compound 151 and reagents, starting materials, and conditions known to those skilled in the art were used to prepare the compounds shown in Table S.

TABLE S

| Compound | Name | Characterization |
|---|---|---|
| Compound | 6-(1-methyl-1H-pyrazol-4-yl)-3-(4-(5- | ¹H NMR (400 MHz, DMSO- |
| 152 | phenethylpyrimidin-2-yl)piperazin-1- | d₆) δ (ppm) 8.83 (s, 1H), 8.23 |
| | yl)pyrazolo[1,5-a]pyridine | (m, 3H), 7.98 (d, J = 0.8 Hz, |
| | | 1H), 7.78 (s, 1H), 7.69 (dd, J = |
| | | 9.2, 1.0 Hz, 1H), 7.37-7.25 |
| | | (m, 3H), 7.25-7.14 (m, 3H), |
| | | 3.92-3.85 (m, 7H), 3.04 (t, J = |
| | | 5.0 Hz, 4H), 2.85 (dd, J = |
| | | 8.8, 6.3 Hz, 2H), 2.75 (dd, J = |
| | | 8.9, 6.2 Hz, 2H). MS (ESI) |
| | | m/z [M + H]⁺ calcd. for |
| | | C₂₇H₂₈N₈, 465.2; found, |
| | | 465.3. HPLC Purity: 254 nm: |
| | | 99.9%. |
| Compound | 3-(4-(5-(2-ethylbutyl)pyrimidin-2- | ¹H NMR (400 MHz, DMSO- |
| 153 | yl)piperazin-1-yl)-6-(1-methyl-1H- | d₆) δ (ppm) 8.83 (s, 1H), 8.23 |
| | pyrazol-4-yl)pyrazolo[1,5-a]pyridine | (s, 3H), 7.98 (s, 1H), 7.79 (s, |
| | | 1H), 7.69 (d, J = 9.2 Hz, 1H), |
| | | 7.33-7.30 (m, 1H), 3.93- |
| | | 3.85 (m, 7H), 3.05 (t, J = 4.9 |
| | | Hz, 4H), 2.36 (d, J = 7.0 Hz, |
| | | 2H), 1.41-1.38 (m, 1H), 1.24- |
| | | 1.21 (m, 4H), 0.86 (t, J = 7.4 |
| | | Hz, 6H). MS (ESI) m/z |
| | | [M + H]⁺ calcd. for C₂₅H₃₂N₈, |
| | | 445.3; found, 445.3; HPLC |
| | | purity: 254 nm: 99.2%. |

### Example 120: Synthesis of Exemplary Compound 154

### 5-benzyl-3-(4-(6-(1-methyl-1H-pyrazol-4-yl)pyrazolo[1,5-a]pyridin-3-yl)piperazin-1-yl)-1,2,4-thiadiazole (Compound 154)

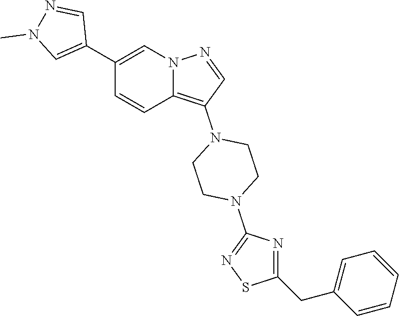 [SMILES: Cn1cc(-c2ccc3c(N4CCN(c5nsc(Cc6ccccc6)n5)CC4)cnn3c2)cn1]

To a solution of 5-benzyl-3-bromo-1,2,4-thiadiazole (Compound S62) (180.7 mg, 0.71 mmol) and 6-(1-methyl-1H-pyrazol-4-yl)-3-piperazin-1-ylpyrazolo[1,5-a]pyridine (Compound 12) (100 mg, 0.35 mmol) in IPA (20 mL) was added DIPEA (91.55 mg, 0.71 mmol). The reaction was stirred for 4 days at 100° C. under nitrogen, then allowed to cool to rt. The resulting mixture was concentrated under reduced pressure. The residue was dissolved in water (50 mL), then extracted with EtOAc (3×50 mL) and dried over anhydrous Na₂SO₄. After filtration, the filtrate was concentrated under reduced pressure. The crude product (100 mg) was purified by Prep-HPLC to afford 28 mg (16%) of 5-benzyl-3-(4-(6-(1-methyl-1H-pyrazol-4-yl)pyrazolo[1,5-a]pyridin-3-yl)piperazin-1-yl)-1,2,4-thiadiazole (Compound 154) as a white solid. ¹H NMR (400 MHZ, DMSO-d₆) δ (ppm) 8.83 (s, 1H), 8.24 (s, 1H), 7.98 (d, J=0.8 Hz, 1H), 7.79 (s, 1H), 7.68 (d, J=9.3 Hz, 1H), 7.49-7.23 (m, 6H), 4.39 (s, 2H), 3.88 (s, 3H), 3.78 (t, J=5.0 Hz, 4H), 3.09 (t, J=5.0 Hz, 4H). MS (ESI) m/z [M+H]⁺ calcd. for C₂₄H₂₄N₈S, 457.1; found, 457.1. HPLC purity: 254 nm: 94.5%.

### Example 121: Synthesis of Exemplary Compound 155

### 2-benzyl-5-(4-(6-(1-methyl-1H-pyrazol-4-yl)pyrazolo[1,5-a]pyridin-3-yl)piperazin-1-yl)-1,3,4-oxadiazole (Compound 155)

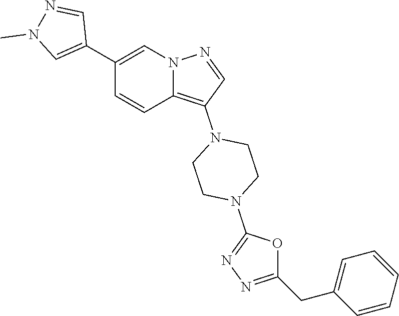 [SMILES: Cn1cc(-c2ccc3c(N4CCN(c5nnc(Cc6ccccc6)o5)CC4)cnn3c2)cn1]

To a solution of 2-benzyl-5-chloro-1,3,4-oxadiazole (Compound S63) (100 mg, 0.51 mmol) and 6-(1-methyl-1H-pyrazol-4-yl)-3-piperazin-1-ylpyrazolo[1,5-a]pyridine (Compound 12) (130.6 mg, 0.46 mmol) in IPA (5 mL) was added DIPEA (199.2 mg, 1.54 mmol) at rt. The reaction was stirred at 90° C. for 5 h, then allowed to cool to rt. The resulting mixture was diluted with H₂O (15 mL) and extracted with EtOAc (3×50 mL). The combined organic layers were dried over anhydrous Na₂SO₄ and filtered. The filtrate was concentrated under reduced pressure. The residue was purified by Prep-TLC (PE/EtOAc 1:1) and the crude product was purified by Prep-HPLC to afford 79.3 mg (35%) of 2-benzyl-5-(4-(6-(1-methyl-1H-pyrazol-4-yl)pyrazolo[1,5-a]pyridin-3-yl)piperazin-1-yl)-1,3,4-oxadiazole (Compound 155) as a yellow solid. ¹H NMR (400 MHZ, DMSO-d₆) δ (ppm) 8.84 (s, 1H), 8.23 (s, 1H), 7.98 (s, 1H), 7.78 (s, 1H), 7.67 (d, J=9.2 Hz, 1H), 7.41-7.25 (m, 6H), 4.12 (s, 2H), 3.87 (s, 3H), 3.65-3.49 (m, 4H), 3.15-3.01 (m, 4H). MS (ESI) m/z [M+H]⁺ calcd. for C₂₄H₂₄N₈O, 441.2; found, 441.4; HPLC purity: 254 nm: 99.5%.

### Example 122: Synthesis of Exemplary Compound 156

### 3-(4-(1-benzyl-1H-1,2,4-triazol-3-yl)piperazin-1-yl)-6-(1-methyl-1H-pyrazol-4-yl)pyrazolo[1,5-a]pyridine (Compound 156)

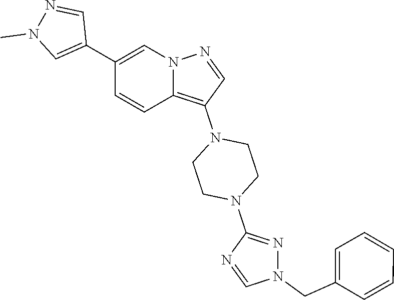 [SMILES: Cn1cc(-c2ccc3c(N4CCN(c5ncn(Cc6ccccc6)n5)CC4)cnn3c2)cn1]

### Step 1. Preparation of 1-benzyl-3-chloro-1H-1,2,4-triazole

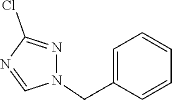 [SMILES: Clc1ncn(Cc2ccccc2)n1]

To a solution of 3-chloro-1H-1,2,4-triazole (950 mg, 9.17 mmol) and benzyl bromide (1.88 g, 11.01 mmol) in DMF (10 mL) was added K₂CO₃ (2.54 g, 18.36 mmol). The reaction was stirred for 4 h at 80° C. under nitrogen. The mixture was allowed to cool to rt, then concentrated under reduced pressure. The resulting mixture was filtered, the filter cake was washed with DCM (3×50 mL). The filtrate was concentrated under reduced pressure. The residue was purified by reversed-phase flash chromatography with C₁₈ silica gel column eluting with 5% to 100% ACN in water (0.1% NH₃·H₂O) afford 1.2 g (68%) of 1-benzyl-3-chloro-1H-1,2,4-triazole as a light-yellow oil. ¹H NMR (400 MHZ, DMSO-d₆) δ (ppm) 8.75 (s, 1H), 7.47-7.20 (m, 5H), 5.41 (d, J=11.9 Hz, 2H). MS (ESI) m/z [M+H]⁺ calcd. for C₉H₈ClN₃, 194.0; found, 194.1.

### Step 2. 3-(4-(1-benzyl-1H-1,2,4-triazol-3-yl)piperazin-1-yl)-6-(1-methyl-1H-pyrazol-4-yl)pyrazolo[1,5-a]pyridine (Compound 156)

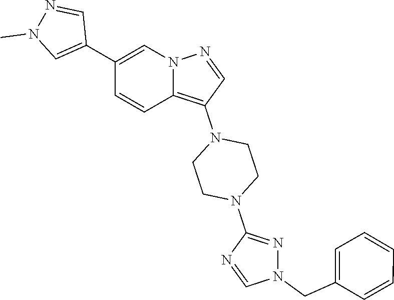 [SMILES: Cn1cc(-c2ccc3c(N4CCN(c5ncn(Cc6ccccc6)n5)CC4)cnn3c2)cn1]

To a solution of 1-benzyl-3-chloro-1,2,4-triazole (300 mg, 1.55 mmol, as prepared in the previous step) and 6-(1-methyl-1H-pyrazol-4-yl)-3-piperazin-1-ylpyrazolo[1,5-a]pyridine (Compound 12) (87.49 mg, 0.31 mmol) in dioxane (4 mL) were added Cs₂CO₃ (1009.6 mg, 3.10 mmol) and Pd-PEPPSI-IPent^{Cl} 2-methylpyridine (195.5 mg, 0.23 mmol). The reaction was stirred for 16 h at 90° C. under nitrogen then the resulting mixture was concentrated under reduced pressure. The residue was diluted with water and extracted with EtOAc (3×50 mL). The combined organic layers were washed with brine (10 mL) and dried over anhydrous Na₂SO₄. After filtration, the filtrate was concentrated under reduced pressure. The crude product was purified by Prep-HPLC to afford 55.2 mg (8%) of 3-(4-(1-benzyl-1H-1,2,4-triazol-3-yl)piperazin-1-yl)-6-(1-methyl-1H-pyrazol-4-yl)pyrazolo[1,5-a]pyridine (Compound 156) as a light yellow solid. ¹H NMR (400 MHZ, DMSO-d₆) δ (ppm) 8.82 (s, 1H), 8.32 (s, 1H), 8.23 (s, 1H), 7.98 (s, 1H), 7.77 (s, 1H), 7.66 (d, J=9.2 Hz, 1H), 7.42-7.25 (m, 6H), 5.24 (s, 2H), 3.87 (s, 3H), 3.45 (t, J=6.3 Hz, 4H), 3.06 (t, J=4.9 Hz, 4H). MS (ESI) m/z [M+H]⁺ calcd. for C₂₄H₂₅N₉, 440.2; found, 440.2; HPLC purity: 254 nm: 99.6%.

### Example 123: Synthesis of Exemplary Compound 157

### 6-(1-methyl-1H-pyrazol-4-yl)-3-(4-(piperidin-1-ylsulfonyl)piperazin-1-yl)pyrazolo[1,5-a]pyridine (Compound 157)

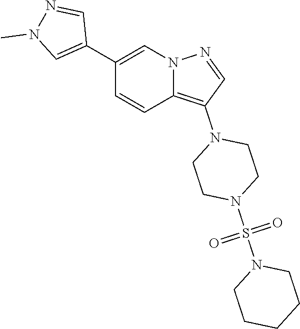 [SMILES: Cn1cc(-c2ccc3c(N4CCN(S(=O)(=O)N5CCCCC5)CC4)cnn3c2)cn1]

To a solution of 6-(1-methyl-1H-pyrazol-4-yl)-3-piperazin-1-ylpyrazolo[1,5-a]pyridine (Compound 12) (80 mg, 0.28 mmol) and DIPEA (73 mg, 0.56 mmol) in DCM (2.0 mL) was added piperidine-1-sulfonyl chloride (56.5 mg, 0.31 mmol) dropwise at 0° C. The reaction was stirred for 16 h at rt. Water (20 mL) was added and the mixture was extracted with EtOAc (2×30 mL). The combined organic layers were dried over anhydrous Na₂SO₄. After filtration, the filtrate was concentrated under reduced pressure. The crude product (240 mg) was purified by Prep-HPLC to afford 97.6 mg (80%) of 6-(1-methyl-1H-pyrazol-4-yl)-3-(4-(piperidin-1-ylsulfonyl)piperazin-1-yl)pyrazolo[1,5-a]pyridine (Compound 157) as a light green solid: ¹H NMR (400 MHZ, DMSO-d₆) δ (ppm) 8.83 (s, 1H), 8.23 (s, 1H), 7.98 (s, 1H), 7.79 (s, 1H), 7.66 (d, J=9.2 Hz, 1H), 7.42-7.28 (m, 1H), 3.87 (s, 3H), 3.32-3.23 (m, 4H), 3.21-3.15 (m, 4H), 3.09-3.01 (m, 4H), 1.38-1.62 (m, 6H). MS (ESI) m/z [M+H]⁺ calcd. for C₂₀H₂₇N₇O₂S, 430.2; found, 430.3; HPLC purity: 254 nm: 99.7%.

### Example 124: Synthesis of Exemplary Compound 158

### N-benzyl-N-methyl-4-(6-(1-methyl-1H-pyrazol-4-yl)pyrazolo[1,5-a]pyridin-3-yl)piperazine-1-sulfonamide (Compound 158)

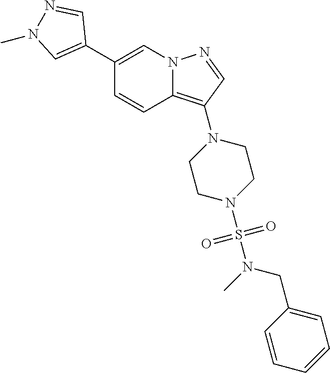 [SMILES: CN(Cc1ccccc1)S(=O)(=O)N1CCN(c2cnn3cc(-c4cnn(C)c4)ccc23)CC1]

To a solution of 6-(1-methyl-1H-pyrazol-4-yl)-3-piperazin-1-ylpyrazolo[1,5-a]pyridine (Compound 12) (400 mg, 1.42 mmol, as prepared in Example 15) dissolved in DCM (15.0 mL) was added N-benzyl-N-methylsulfamoyl chloride (342 mg, 1.56 mmol) and Et₃N (287 mg, 2.83 mmol). The reaction was stirred for 2 h at rt. The resulting mixture was concentrated under reduced pressure, diluted with water (20 mL) and extracted with EtOAc (3×25 mL). The organic layers were combined and concentrated under reduced pressure. The residue was applied onto a silica gel column with EtOAc/PE (1/1). The crude product was purified by Prep-HPLC to afford 200 mg (30%) of N-benzyl-N-methyl-4-(6-(1-methyl-1H-pyrazol-4-yl)pyrazolo[1,5-a]pyridin-3-yl)piperazine-1-sulfonamide (Compound 158) as a light yellow solid. ¹H NMR (400 MHZ, DMSO-d₆) δ (ppm) 8.84 (s, 1H), 8.24 (s, 1H), 7.98 (s, 1H), 7.80 (s, 1H), 7.67 (d, J=9.2 Hz, 1H), 7.44-7.30 (m, 6H), 4.39 (s, 2H), 3.87 (s, 3H), 3.38 (t, J=5.0 Hz, 4H), 3.08 (dd, J=6.0, 3.8 Hz, 4H), 2.73 (s, 3H). MS (ESI) m/z [M+H]⁺ calcd. for C₂₃H₂₇N₇O₂S, 466.2; found, 466.1; HPLC purity: 254 nm: 99.9%.

### Example 125: Synthesis of Exemplary Compound 159

### N-benzyl-4-[6-(1-methyl-1H-pyrazol-4-yl)pyrazolo[1,5-a]pyridin-3-yl]piperazine-1-sulfonamide (Compound 159)

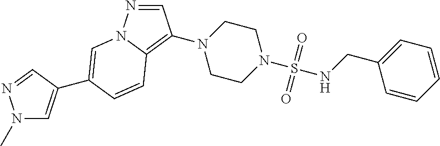 [SMILES: Cn1cc(-c2ccc3c(N4CCN(S(=O)(=O)NCc5ccccc5)CC4)cnn3c2)cn1]

### Step 1. Synthesis of benzylsulfamic acid

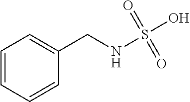 [SMILES: O=S(=O)(O)NCc1ccccc1]

To a stirred solution of benzylamine (2.76 g, 25.75 mmol) in DCM (30 mL) was added chlorosulfonic acid (1 g, 8.58 mmol) dropwise at 0° C. The resulting mixture was stirred for 30 min at 0° C. The precipitated solids were collected by filtration and washed with DCM (2×10 mL) to afford 2.5 g of benzylsulfamic acid as a white solid. MS (ESI) m/z [M+H]⁺ calcd. for C₇H9NO₃S, 188.03; [M−H]⁻ found, 186. 1.

### Step 2. Synthesis of benzylsulfamoyl chloride

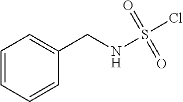 [SMILES: O=S(=O)(Cl)NCc1ccccc1]

To a stirred solution of benzylsulfamic acid (300 mg, 1.60 mmol, as prepared in the previous step) in toluene (10 mL) was added PCl₅ (333.7 mg, 1.60 mmol) in portions at 0° C. The reaction was stirred for 2 h at 75° C. then allowed to cool to rt and concentrated under reduced pressure to afford 430 mg of benzylsulfamoyl chloride as a colorless oil. MS (ESI) m/z [M+H]⁺ calcd. for C₇H₈ClNO₂S, 206.1; found, 206.2.

### Step 3. Synthesis of N-benzyl-4-[6-(1-methyl-1H-pyrazol-4-yl)pyrazolo[1,5-a]pyridin-3-yl]piperazine-1-sulfonamide (Compound 159)

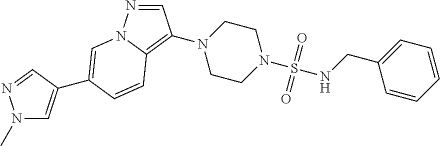 [SMILES: Cn1cc(-c2ccc3c(N4CCN(S(=O)(=O)NCc5ccccc5)CC4)cnn3c2)cn1]

To a stirred solution of 6-(1-methyl-1H-pyrazol-4-yl)-3-piperazin-1-ylpyrazolo[1,5-a]pyridine (Compound 12) (494.2 mg, 1.75 mmol) in DCM (20 mL) was added DIPEA (565.6 mg, 4.38 mmol) and N-benzylsulfamoyl chloride (300 mg, 1.46 mmol, as prepared in the previous step) dropwise at 0° C. The reaction was stirred for 2 h at rt. The resulting mixture was concentrated under reduced pressure then the residue was dissolved in water (100 mL) and extracted with DCM (2×100 mL). The combined organic layers were washed with brine (10 mL), dried over anhydrous Na₂SO₄. After filtration, the filtrate was concentrated under reduced pressure. The residue was purified by Prep-TLC (DCM/MeOH 10:1) to afford crude product. The crude product was purified by Prep-HPLC to afford 60.6 mg (9%) of N-benzyl-4-[6-(1-methyl-1H-pyrazol-4-yl)pyrazolo[1,5-a]pyridin-3-yl]piperazine-1-sulfonamide (Compound 159) as a light yellow solid. ¹H NMR (400 MHZ, DMSO-d₆) δ (ppm) 8.85-8.81 (m, 1H), 8.23 (s, 1H), 8.00-7.87 (m, 2H), 7.74 (s, 1H), 7.66-7.61 (m, 1H), 7.43-7.24 (m, 6H), 4.15 (s, 2H), 3.87 (s, 3H), 3.24-3.17 (m, 4H), 3.02-2.92 (m, 4H). MS (ESI) m/z [M+H]⁺ calcd. for C₂₆H₃₁N₈O, 452.2; found, 452.1, HPLC purity: 254 nm: 98.5%.

### Example 126: Synthesis of Exemplary Compound 160

### 6-(1-methyl-1H-pyrazol-4-yl)-3-(4-(phenethylsulfonyl)piperazin-1-yl)pyrazolo[1,5-a]pyridine (Compound 160)

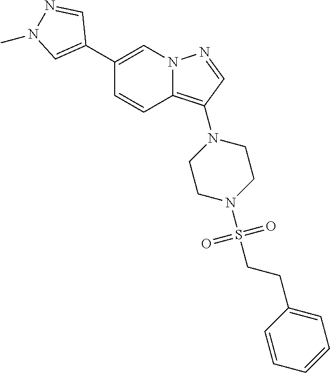 [SMILES: Cn1cc(-c2ccc3c(N4CCN(S(=O)(=O)CCc5ccccc5)CC4)cnn3c2)cn1]

To a solution of 6-(1-methyl-1H-pyrazol-4-yl)-3-piperazin-1-ylpyrazolo[1,5-a]pyridine (Compound 12) (100 mg, 0.35 mmol) in THF (3 mL) were added DIPEA (91.5 mg, 0.71 mmol) and 2-phenylethanesulfonyl chloride (72.4 mg, 0.35 mmol). The reaction was stirred for 1 h at 0° C. under nitrogen atmosphere then concentrated under reduced pressure. The residue was diluted with water and extracted with EtOAc (3×30 mL). The combined organic layers were washed with brine (20 mL), dried over anhydrous Na₂SO₄. After filtration, the filtrate was concentrated under reduced pressure. The crude product was purified by Prep-HPLC to afford 125.5 mg (78%) of 6-(1-methyl-1H-pyrazol-4-yl)-3-(4-(phenethylsulfonyl)piperazin-1-yl)pyrazolo[1,5-a]pyridine (Compound 160) as a light yellow solid. ¹H NMR (400 MHZ, DMSO-d₆) δ (ppm) 8.87 (s, 1H), 8.77 (s, 2H), 8.25 (s, 1H), 7.99 (s, 1H), 7.88 (s, 1H), 7.76 (d, J=9.2 Hz, 1H), 7.41-7.30 (m, 5H), 7.30-7.20 (m, 1H), 4.03 (s, 2H), 4.00-3.93 (m, 2H), 3.90-3.83 (m, 5H), 3.45 (t, J=5.4 Hz, 2H). MS (ESI) m/z [M+H]⁺ calcd. for C₂₃H₂₆N₆O₂, 451.2, found, 451.1; HPLC purity: 254 nm: 99.5%.

### Example 127: Synthesis of Exemplary Compound 161

### N-benzyl-4-(6-(1-methyl-1H-pyrazol-4-yl)pyrazolo[1,5-a]pyridin-3-yl)piperazine-1-carboxamide (Compound 161)

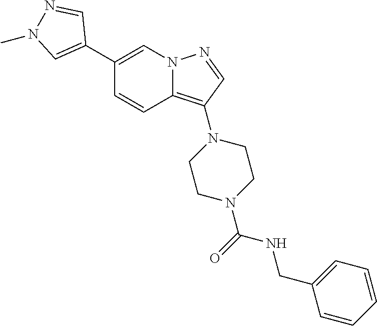 [SMILES: Cn1cc(-c2ccc3c(N4CCN(C(=O)NCc5ccccc5)CC4)cnn3c2)cn1]

To a solution of 6-(1-methyl-1H-pyrazol-4-yl)-3-piperazin-1-ylpyrazolo[1,5-a]pyridine (Compound 12) (100 mg, 0.35 mmol) in DCM (3 mL) were added DIPEA (91.5 mg, 0.71 mmol) and (isocyanatomethyl)benzene (47.1 mg, 0.35 mmol). The reaction was stirred for 1 h at 0° C. under nitrogen then concentrated under reduced pressure. The residue was diluted with water and extracted with EtOAc (3×30 mL). The combined organic layers were washed with brine (20 mL) and dried over anhydrous Na₂SO₄. After filtration, the filtrate was concentrated under reduced pressure. The crude product was purified by Prep-HPLC to afford 51.2 mg (35%) of N-benzyl-4-(6-(1-methyl-1H-pyrazol-4-yl)pyrazolo[1,5-a]pyridin-3-yl)piperazine-1-carboxamide (Compound 161) as a light grey solid. ¹H NMR (400 MHZ, DMSO-d₆) δ (ppm) 8.83 (s, 1H), 8.23 (s, 1H), 7.98 (s, 1H), 7.78 (s, 1H), 7.70-7.63 (m, 1H), 7.36-7.25 (m, 1H), 7.26-7.15 (m, 2H), 4.28 (d, J=5.7 Hz, 2H), 3.87 (s, 3H), 3.57-3.50 (m, 4H), 2.96 (t, J=4.9 Hz, 4H). MS (ESI) m/z [M+H]⁺ calcd. for C₂₃H₂₅N₇O, 416.2; found, 416.2; HPLC purity: 254 nm: 99.8%.

### Example 128: Synthesis of Exemplary Compound 162

### N-benzyl-N-methyl-4-(6-(1-methyl-1H-pyrazol-4-yl)pyrazolo[1,5-a]pyridin-3-yl)piperazine-1-carboxamide (Compound 162)

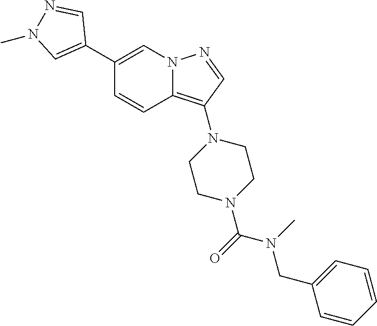 [SMILES: CN(Cc1ccccc1)C(=O)N1CCN(c2cnn3cc(-c4cnn(C)c4)ccc23)CC1]

To a solution of N-benzyl-4-(6-(1-methyl-1H-pyrazol-4-yl)pyrazolo[1,5-a]pyridin-3-yl)piperazine-1-carboxamide (Compound 161) (150 mg, 0.36 mmol) in DMF (10 mL) was added NaH (28.8 mg, 0.72 mmol, 60% mineral oil dispersion) at 0° C. The above mixture was stirred for 0.5 h at 0° C. then CH₃I (56.37 mg, 0.10 mmol) was added and stirred for 1 h at 0° C. under nitrogen. The resulting mixture was concentrated under reduced pressure, diluted with water (50 mL), extracted with EtOAc (3×50 mL) and dried over anhydrous Na₂SO₄. After filtration, the filtrate was concentrated under reduced pressure. The residue was purified by silica gel column chromatography eluting with PE/EtOAc (9:1) to afford crude product. The crude product was purified by Prep-HPLC to afford 94.5 mg (61%) of N-benzyl-N-methyl-4-(6-(1-methyl-1H-pyrazol-4-yl)pyrazolo[1,5-a]pyridin-3-yl)piperazine-1-carboxamide (Compound 162) as a green solid. ¹H NMR (400 MHZ, DMSO-d₆) δ (ppm) 8.87 (s, 1H), 8.77 (s, 2H), 8.25 (s, 1H), 7.99 (s, 1H), 7.88 (s, 1H), 7.76 (d, J=9.2 Hz, 1H), 7.41-7.30 (m, 5H), 7.30-7.20 (m, 1H), 4.03 (s, 2H), 4.00-3.93 (m, 2H), 3.90-3.83 (m, 5H), 3.45 (t, J=5.4 Hz, 2H). MS (ESI) m/z [M+H]⁺ calcd. for C₂₄H₂₇N₇O, 430.2; found, 430.1; HPLC purity: 254 nm: 99.7%.

### Example 129: Synthesis of Exemplary Compound 163

### (4-(6-(1-methyl-1H-pyrazol-4-yl)pyrazolo[1,5-a]pyridin-3-yl)piperazin-1-yl)(piperidin-1-yl)methanone (Compound 163)

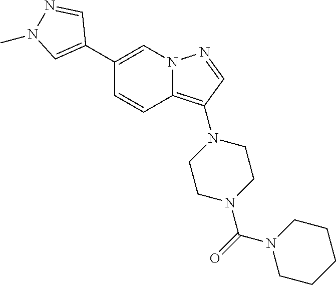 [SMILES: Cn1cc(-c2ccc3c(N4CCN(C(=O)N5CCCCC5)CC4)cnn3c2)cn1]

To a solution of 6-(1-methyl-1H-pyrazol-4-yl)-3-piperazin-1-ylpyrazolo[1,5-a]pyridine (Compound 12) (60.0 mg, 0.21 mmol) in DCM (2.0 mL) was added DIPEA (54.9 mg, 0.43 mmol) and piperidine-1-carbonyl chloride (34.5 mg, 0.23 mmol) dropwise at 0° C. The reaction was stirred for 1 h at rt, water was added (20 mL) and extracted with EtOAc (2×30 mL). The combined organic layers were dried over anhydrous Na₂SO₄. After filtration, the filtrate was concentrated under reduced pressure. The crude product (200 mg) was purified by Prep-HPLC to afford 72.0 mg (86%) of (4-(6-(1-methyl-1H-pyrazol-4-yl)pyrazolo[1,5-a]pyridin-3-yl)piperazin-1-yl) (piperidin-1-yl)methanone (Compound 163) as a yellow solid. ¹H NMR (400 MHZ, DMSO-d₆) δ (ppm) 8.82 (d, J=1.5 Hz, 1H), 8.23 (s, 1H), 7.97 (s, 1H), 7.77 (s, 1H), 7.65 (d, J=9.2 Hz, 1H), 7.32 (dd, J=9.3, 1.5 Hz, 1H), 3.87 (s, 3H), 3.33-3.30 (m, 4H), 3.16 (t, J=5.1 Hz, 4H), 2.99 (t, J=4.8 Hz, 4H), 1.60-1.46 (m, 6H). MS (ESI) m/z [M+H]⁺ calcd. for C₂₁H27NO, 394.2; found, 394.3; HPLC purity: 254 nm: 99.7%.

### Example 130: Synthesis of Exemplary Compound 164

### 2-(2-(4-(6-(1-methyl-1H-pyrazol-4-yl)pyrazolo[1,5-a]pyridin-3-yl)piperazin-1-yl)pyrimidin-5-yl)-1-phenylethan-1-ol (Compound 164)

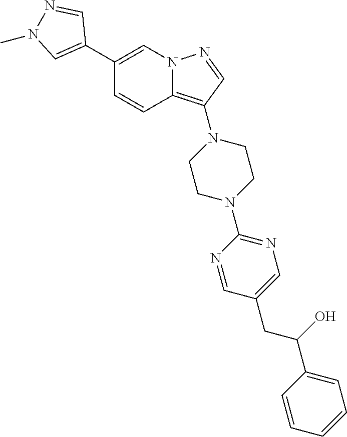 [SMILES: Cn1cc(-c2ccc3c(N4CCN(c5ncc(CC(O)c6ccccc6)cn5)CC4)cnn3c2)cn1]

### Step 1. Preparation of 3-(4-(5-iodopyrimidin-2-yl)piperazin-1-yl)-6-(1-methyl-1H-pyrazol-4-yl)pyrazolo[1,5-a]pyridine

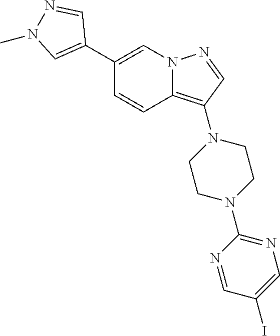 [SMILES: Cn1cc(-c2ccc3c(N4CCN(c5ncc(I)cn5)CC4)cnn3c2)cn1]

To a solution of 6-(1-methyl-1H-pyrazol-4-yl)-3-piperazin-1-ylpyrazolo[1,5-a]pyridine (Compound 12) (1 g, 3.54 mmol) and 2-chloro-5-iodopyrimidine (1.02 g, 4.25 mmol) in IPA (4 mL) was added DIPEA (1.85 mL, 10.6 mmol). The reaction was stirred for 16 h at 90° C. under nitrogen then concentrated under reduced pressure. The precipitated solids were collected by filtration and washed with MeOH (3×50 mL) to afford 1.4 g (81%) of 3-(4-(5-iodopyrimidin-2-yl)piperazin-1-yl)-6-(1-methyl-1H-pyrazol-4-yl)pyrazolo[1,5-a]pyridine as a yellow solid. ¹H NMR (400 MHZ, DMSO-d₆) δ (ppm) 8.83 (s, 1H), 8.55 (s, 2H), 8.23 (s, 1H), 7.98 (s, 1H), 7.78 (s, 1H), 7.69 (d, J=9.2 Hz, 1H), 7.34 (d, J=9.2 Hz, 1H), 3.97-3.84 (m, 7H), 3.05 (t, J=5.0 Hz, 4H); MS (ESI) m/z [M+H]⁺ calcd. for C₁₉H₁₉IN₈, 487.1; found, 487.3.

### Step 2. Preparation of 2-(2-(4-(6-(1-methyl-1H-pyrazol-4-yl)pyrazolo[1,5-a]pyridin-3-yl)piperazin-1-yl)pyrimidin-5-yl)-1-phenylethan-1-one

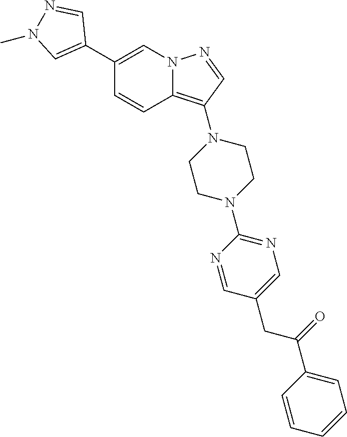 [SMILES: Cn1cc(-c2ccc3c(N4CCN(c5ncc(CC(=O)c6ccccc6)cn5)CC4)cnn3c2)cn1]

To a solution of 3-(4-(5-iodopyrimidin-2-yl)piperazin-1-yl)-6-(1-methyl-1H-pyrazol-4-yl)pyrazolo[1,5-a]pyridine (400 mg, 0.82 mmol, as prepared in the previous step) and acetophenone (118.5 mg, 0.99 mmol) in dioxane (10 mL) were added XPhos Pd G4 (139.2 mg, 1.65 mmol) and NaOtBu (158.1 mg, 1.65 mmol). The reaction was stirred for 16 h at 100° C. under nitrogen then concentrated under reduced pressure. The residue was diluted with water and extracted with EtOAc (3×30 mL). The combined organic layers were washed with brine (10 mL) and dried over anhydrous Na₂SO₄. After filtration, the filtrate was concentrated under reduced pressure. The residue was purified by silica gel column chromatography eluting with PE/EtOAc (1:1) to afford 200 mg (51%) of 2-(2-(4-(6-(1-methyl-1H-pyrazol-4-yl)pyrazolo[1,5-a]pyridin-3-yl)piperazin-1-yl)pyrimidin-5-yl)-1-phenylethan-1-one as yellow solid. ¹H NMR (400 MHZ, DMSO-d₆) δ (ppm) 8.86 (m, 2H), 8.27 (m, 2H), 7.97 (m, 2H), 7.86-7.53 (m, 5H), 7.39-7.21 (m, 2H), 4.14 (t, J=5.1 Hz, 2H), 4.05-3.90 (m, 2H), 3.88 (s, 3H), 3.51 (s, 2H), 3.14-2.98 (m, 4H). MS (ESI) m/z [M+H]⁺ calcd. for C₂₇H₂₆N₈O, 478.2; found, 478.1.

### Step 3. 2-(2-(4-(6-(1-methyl-1H-pyrazol-4-yl)pyrazolo[1,5-a]pyridin-3-yl)piperazin-1-yl)pyrimidin-5-yl)-1-phenylethan-1-ol (Compound 164)

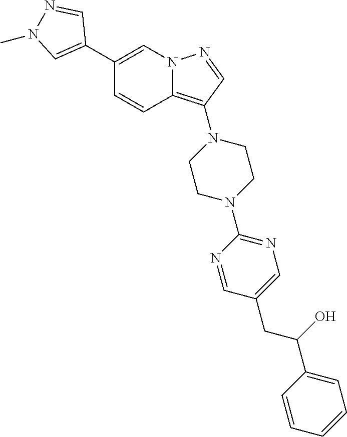 [SMILES: Cn1cc(-c2ccc3c(N4CCN(c5ncc(CC(O)c6ccccc6)cn5)CC4)cnn3c2)cn1]

To a solution of 2-(2-(4-(6-(1-methyl-1H-pyrazol-4-yl)pyrazolo[1,5-a]pyridin-3-yl)piperazin-1-yl)pyrimidin-5-yl)-1-phenylethan-1-one (100 mg, 0.21 mmol, as prepared in the previous step) in DCM (2 mL) and MeOH (4 mL) was added NaBH₄ (23.7 mg, 0.63 mmol). The reaction was stirred for 0.5 h at rt under nitrogen then concentrated under reduced pressure and quenched with MeOH at rt. The resulting mixture was concentrated under reduced pressure. The residue was diluted with water and extracted with EtOAc (3×mL). The combined organic layers were washed with brine (10 mL), dried over anhydrous Na₂SO₄. After filtration, the filtrate was concentrated under reduced pressure. The crude product was purified by Prep-HPLC to afford 24.4 mg (24%) of 2-(2-(4-(6-(1-methyl-1H-pyrazol-4-yl)pyrazolo[1,5-a]pyridin-3-yl)piperazin-1-yl)pyrimidin-5-yl)-1-phenylethan-1-ol (Compound 164) as a yellow solid. ¹H NMR (400 MHZ, DMSO-d₆) δ (ppm) 8.82 (s, 1H), 8.22 (s, 1H), 8.13 (s, 2H), 7.97 (s, 1H), 7.77 (s, 1H), 7.69-7.67 (d, J=8 Hz, 1H), 7.34-7.31 (m, 5H), 7.25-7.22 (m, 1H), 5.37-5.36 (d, J=4.4 Hz, 1H), 4.69-4.67 (m, 1H), 3.88-3.86 (m, 7H), 3.04-3.02 (t, J=5 Hz, 4H), 2.75-2.66 (m, 2H). MS (ESI) m/z [M+H]⁺ calcd. for C₂₇H₂₈N₈O, 481.2; found, 481.2; HPLC purity: 254 nm: 99.2%.

### Example 131: Synthesis of Exemplary Compound 165

### 3-(2-(4-(6-(1-methyl-1H-pyrazol-4-yl)pyrazolo[1,5-a]pyridin-3-yl)piperazin-1-yl)pyrimidin-5-yl)-1-phenylpropan-1-ol (Compound 165)

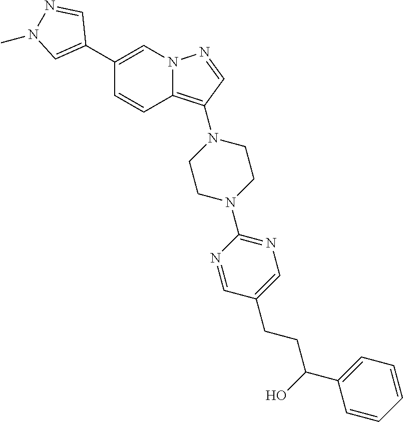 [SMILES: Cn1cc(-c2ccc3c(N4CCN(c5ncc(CCC(O)c6ccccc6)cn5)CC4)cnn3c2)cn1]

### Step 1. Preparation of 3-(2-chloropyrimidin-5-yl)-1-phenylprop-2-en-1-one

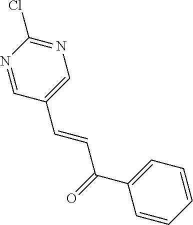 [SMILES: O=C(/C=C/c1cnc(Cl)nc1)c1ccccc1]

To a stirred solution of 1-phenyl-2-(triphenyl-25-phosphaneylidene) ethan-1-one (6.94 g, 15.05 mmol) in THF (150 mL) were added 2-chloropyrimidine-5-carbaldehyde (1.65 g, 11.58 mmol) and Cs₂CO₃ (4.15 g, 12.73 mmol) at rt. The reaction was stirred overnight at rt. The resulting mixture was filtered, and the filter cake was washed with EtOAc (3×100 mL). The filtrate was concentrated under reduced pressure. The residue was purified by silica gel column chromatography eluting with DCM/MeOH (30:1) to afford 910 mg (32%) of 3-(2-chloropyrimidin-5-yl)-1-phenylprop-2-en-1-one as a white solid. ¹H NMR (400 MHZ, DMSO-d₆) δ (ppm) 9.31 (s, 2H), 8.28-8.16 (m, 3H), 7.78-7.68 (m, 2H), 7.64-7.58 (m, 2H). MS (ESI) m/z [M+H]⁺ calcd. for C₁₃H₉ClN₂O, 245.0; found, 245.1.

### Step 2. Preparation of 3-(2-(4-(6-(1-methyl-1H-pyrazol-4-yl)pyrazolo[1,5-a]pyridin-3-yl)piperazin-1-yl)pyrimidin-5-yl)-1-phenylprop-2-en-1-one

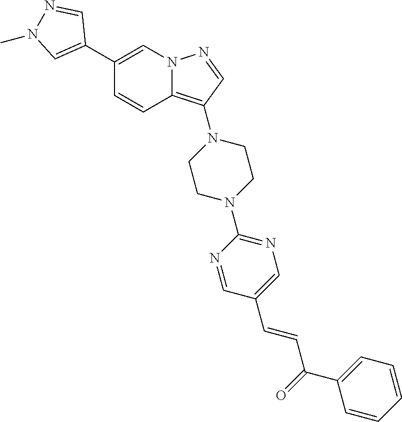 [SMILES: Cn1cc(-c2ccc3c(N4CCN(c5ncc(/C=C/C(=O)c6ccccc6)cn5)CC4)cnn3c2)cn1]

To a stirred solution of 3-(2-chloropyrimidin-5-yl)-1-phenylprop-2-en-1-one (300 mg, 1.23 mmol, as prepared in the previous step) and 6-(1-methyl-1H-pyrazol-4-yl)-3-piperazin-1-ylpyrazolo[1,5-a]pyridine (Compound 12) (346.2 mg, 1.23 mmol) in IPA (10 mL) was added DIPEA (316.9 mg, 2.45 mmol) at rt. The reaction was stirred for 16 h at 90° C. then concentrated under reduced pressure. The residue was purified by trituration with MeOH (300 mL) to afford 550 mg (91%) of 3-(2-(4-(6-(1-methyl-1H-pyrazol-4-yl)pyrazolo[1,5-a]pyridin-3-yl)piperazin-1-yl)pyrimidin-5-yl)-1-phenylprop-2-en-1-one as a yellow solid. ¹H NMR (400 MHz, DMSO-d₆) δ (ppm) 8.95 (s, 2H), 8.85 (s, 1H), 8.25 (s, 1H), 8.15 (d, J=7.4 Hz, 2H), 7.99 (s, 1H), 7.91 (d, J=15.7 Hz, 1H), 7.81 (s, 1H), 7.74-7.64 (m, 3H), 7.58 (t, J=7.6 Hz, 2H), 7.35 (dd, J=9.2, 1.5 Hz, 1H), 4.10-4.03 (m, 4H), 3.88 (s, 3H), 3.19-3.03 (m, 4H). MS (ESI) m/z [M+H]⁺ calcd. for C₂₈H₂₆N₈O, 491.2; found, 491.3.

### Step 3. Preparation of 3-(2-(4-(6-(1-methyl-1H-pyrazol-4-yl)pyrazolo[1,5-a]pyridin-3-yl)piperazin-1-yl)pyrimidin-5-yl)-1-phenylpropan-1-ol (Compound 165)

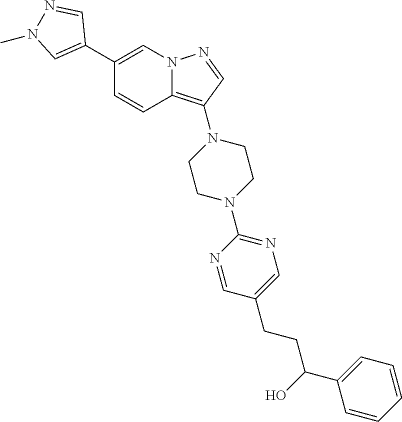 [SMILES: Cn1cc(-c2ccc3c(N4CCN(c5ncc(CCC(O)c6ccccc6)cn5)CC4)cnn3c2)cn1]

To a stirred solution of 3-(2-(4-(6-(1-methyl-1H-pyrazol-4-yl)pyrazolo[1,5-a]-498-yridine-3-yl)piperazin-1-yl)pyrimidin-5-yl)-1-phenylprop-2-en-1-one (500 mg, 1.02 mmol, as prepared in the previous step) in MeOH (8 mL) was added Pd/C (21.7 mg, 0.20 mmol) at rt. The reaction was stirred for 24 h at rt under hydrogen atmosphere. The precipitated solids were collected by filtration and washed with DCM (3×80 mL). The combined filtrates were concentrated under reduced pressure. The residue was purified by Prep-TLC (DCM/MeOH 20:1) and then purified by Prep-HPLC to afford 77.3 mg (15%) of 3-(2-(4-(6-(1-methyl-1H-pyrazol-4-yl)pyrazolo[1,5-a]pyridin-3-yl)piperazin-1-yl)pyrimidin-5-yl)-1-phenylpropan-1-ol (Compound 165) as a light yellow solid. ¹H NMR (400 MHZ, DMSO-d₆) δ (ppm) 8.83 (s, 1H), 8.27 (s, 2H), 8.23 (s, 1H), 7.98 (s, 1H), 7.78 (s, 1H), 7.69 (d, J=9.2 Hz, 1H), 7.38-7.28 (m, 5H), 7.28-7.18 (m, 1H), 5.29 (d, J=3.6 Hz, 1H), 4.56-4.47 (m, 1H), 3.92-3.84 (m, 7H), 3.05 (t, J=4.9 Hz, 4H), 2.60-2.53 (m, 1H), 2.48-2.38 (m, 1H), 1.98-1.73 (m, 2H). MS (ESI) m/z [M+H]⁺ calcd. for C₂₈H₃₀N₈O, 495.3; found, 495.25; HPLC purity: 254 nm: 99.3%.

### Example 132: Synthesis of Exemplary Compound 166

### 6-(1-methyl-1H-pyrazol-4-yl)-3-(4-(5-phenoxypyrimidin-2-yl)piperazin-1-yl)pyrazolo[1,5-a]pyridine (Compound 166)

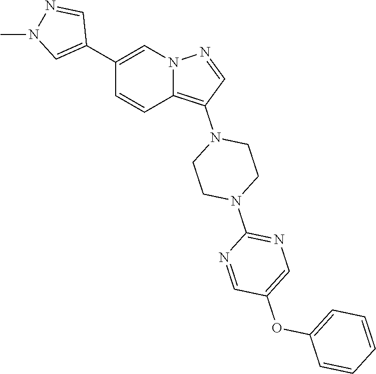 [SMILES: Cn1cc(-c2ccc3c(N4CCN(c5ncc(Oc6ccccc6)cn5)CC4)cnn3c2)cn1]

### Step 1. Preparation of 2-chloro-5-phenoxypyrimidine

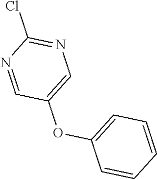 [SMILES: Clc1ncc(Oc2ccccc2)cn1]

To a solution of 2-chloropyrimidin-5-ol (2.0 g, 15.3 mmol) and phenylboronic acid (5.6 g, 46.0 mmol) in DCM (150 mL) was added Et₃N (4.65 g, 46.0 mmol) and Cu(OAc)₂ (5.57 g, 30.6 mmol). The reaction was stirred for 60 h at rt under air atmosphere. The resulting mixture was filtered, and the filter cake was washed with DCM (2×10 mL). The filtrate was concentrated under reduced pressure. The residue was purified by silica gel column chromatography eluting with PE/EtOAc (5:1) to afford 1.8 g (54%) of 2-chloro-5-phenoxypyrimidine as a yellow oil. ¹H NMR (400 MHZ, DMSO-d₆) δ (ppm) 8.60 (s, 2H), 7.50-7.35 (m, 2H), 7.30-7.19 (m, 1H), 7.23-7.08 (m, 2H). MS (ESI) m/z [M+H]⁺ calcd. For C₁₀H₇ClN₂O, 207.0; found, 207.1.

### Step 2. Preparation of 6-(1-methyl-1H-pyrazol-4-yl)-3-(4-(5-phenoxypyrimidin-2-yl)piperazin-1-yl)pyrazolo[1,5-a]pyridine (Compound 166)

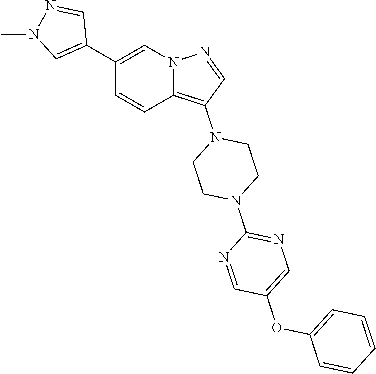 [SMILES: Cn1cc(-c2ccc3c(N4CCN(c5ncc(Oc6ccccc6)cn5)CC4)cnn3c2)cn1]

To a solution of 6-(1-methyl-1H-pyrazol-4-yl)-3-(piperazin-1-yl)pyrazolo[1,5-a]pyridine (Compound 12) (100 mg, 0.35 mmol) in IPA (2.0 mL) were added DIPEA (91.6 mg, 0.71 mmol) and 2-chloro-5-phenoxypyrimidine (87.8 mg, 0.43 mmol, as prepared in the previous step). The reaction was stirred for 16 h at 110° C. then allowed to cool to rt. The resulting mixture was concentrated under reduced pressure. The residue was purified by Prep-HPLC to afford 101.7 mg (63%) of 6-(1-methyl-1H-pyrazol-4-yl)-3-(4-(5-phenoxypyrimidin-2-yl)piperazin-1-yl)pyrazolo[1,5-a]pyridine (Compound 166) as a grey solid. ¹H NMR (400 MHz, DMSO-d₆) δ (ppm) 8.84 (t, J=1.2 Hz, 1H), 8.35 (s, 2H), 8.24 (s, 1H), 7.98 (d, J=0.8 Hz, 1H), 7.80 (s, 1H), 7.71 (dd, J=9.2, 1.0 Hz, 1H), 7.41-7.31 (m, 3H), 7.14-7.05 (m, 1H), 7.03-6.95 (m, 2H), 3.93 (t, J=5.0 Hz, 4H), 3.88 (s, 3H), 3.09 (t, J=5.0 Hz, 4H). MS (ESI) m/z [M+H]⁺ calcd. for C₂₅H₂₄N₈O, 453.2; found, 453.3; HPLC purity: 254 nm: 99.5%.

### Example 133: Synthesis of Exemplary Compound 167

### Synthesis of 1-(2-{4-[6-(1-methyl-1H-pyrazol-4-yl)pyrazolo[1,5-a]pyridin-3-yl]piperazin-1-yl}pyrimidin-5-yl)-2-phenylethanol (Compound 167)

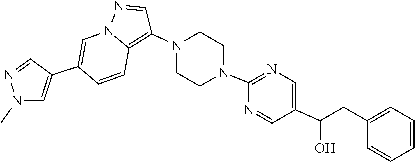 [SMILES: Cn1cc(-c2ccc3c(N4CCN(c5ncc(C(O)Cc6ccccc6)cn5)CC4)cnn3c2)cn1]

To a stirred solution of 1-(2-chloropyrimidin-5-yl)-2-phenylethanol (Compound S58) (80 mg, 0.34 mmol) and 1-[6-(1-methylpyrazol-4-yl)pyrazolo[1,5-a]pyridin-3-yl]piperazine (Compound 12) (115.5 mg, 0.41 mmol) in IPA (15 mL) was added DIPEA (132.2 mg, 1.02 mmol) dropwise at rt. The reaction was stirred overnight at 90° C. then concentrated under vacuum. The residue was purified by Prep-TLC (DCM/MeOH 10:1) to afford crude product. The crude product was purified by Prep-HPLC to afford 110.4 mg (67%) of 1-(2-{4-[6-(1-methyl-1H-pyrazol-4-yl)pyrazolo[1,5-a]pyridin-3-yl]piperazin-1-yl}pyrimidin-5-yl)-2-phenylethanol (Compound 167) as a white solid. ¹H NMR (400 MHz, DMSO-d₆) δ (ppm) 8.83 (s, 1H), 8.26 (s, 2H), 8.24 (s, 1H), 7.98 (s, 1H), 7.78 (s, 1H), 7.69 (d, J=9.2 Hz, 1H), 7.34 (dd, J=9.2, 1.5 Hz, 1H), 7.29-7.21 (m, 2H), 7.18 (d, J=7.0 Hz, 3H), 5.33 (d, J=4.5 Hz, 1H), 4.72-4. 70 (m, 1H), 3.98-3.85 (m, 7H), 3.10-2.97 (m, 5H), 2.96-2.86 (m, 1H). S (ESI) m/z [M+H]⁺ calcd. for C₂₇H₂₈N₈, 480.24; found, 480.34. HPLC Purity: 254 nm: 99.3%.

Using the procedures described in Example 133, Compound 167 and reagents, starting materials, and conditions known to those skilled in the art were used to prepare the compounds shown in Table T.

TABLE T

| Compound | Name | Characterization |
|---|---|---|
| Compound | 1-(2-(4-(6-(1-methyl-1H-pyrazol-4- | ¹H NMR (400 MHz, DMSO- |
| 168 | yl)pyrazolo[1,5-a]pyridin-3-yl)piperazin- | d₆) δ (ppm) 8.83 (s, 1H), 8.33 |
| | 1-yl)pyrimidin-5-yl)pentan-1-ol | (s, 2H), 8.23 (s, 1H), 7.97 (s, |
| | | 1H), 7.78 (s, 1H), 7.69 (d, J = |
| | | 8.8 Hz, 1H), 7.33 (d, J = 7.2 |
| | | Hz, 1H), 5.12 (d, J = 4.4 Hz, |
| | | 1H), 4.42 (dd, J = 6.4 Hz, 4.4 |
| | | Hz, 1H), 3.96-3.85 (m, 7H), |
| | | 3.05 (t, J = 4.8 Hz, 4H), 1.72- |
| | | 1.55 (m, 2H), 1.33-1.23 (m, |
| | | 3H), 1.08-1.22 (m, 1H), 0.86 |
| | | (t, J = 6.8 Hz, 3H). MS (ESI) |
| | | m/z [M + H]⁺ calcd. for |
| | | C₂₄H₃₀N₈O, 447.3; found, |
| | | 447.2; HPLC purity: 254 nm: |
| | | 99.5%. |
| Compound | 3-(4-(5-(furan-3-ylmethyl)pyrimidin-2- | ¹H NMR (400 MHz, DMSO- |
| 169 | yl)piperazin-1-yl)-6-(1-methyl-1H- | d₆) δ (ppm) 8.83 (s, 1H), 8.30 |
| | pyrazol-4-yl)pyrazolo[1,5-a]pyridine | (s, 2H), 8.23 (s, 1H), 7.98 (s, |
| | | 1H), 7.78 (s, 1H), 7.69 (d, J = |
| | | 9.3 Hz, 1H), 7.59 (s, 1H), 7.48 |
| | | (s, 1H), 7.33 (d, J = 9.3Hz, |
| | | 1H), 6.39 (s, 1H), 3.90 (t, J = |
| | | 4.9 Hz, 4H), 3.88 (s, 3H), 3.59 |
| | | (s, 2H), 3.05 (t, J = 5.0 Hz, |
| | | 4H). MS (ESI) m/z [M + H]⁺ |
| | | calcd. for C₂₄H₂₄N₈O, 441.2; |
| | | found, 441.2; HPLC purity: |
| | | 254 nm: 99.8%. |
| Compound | 2-((2-(4-(6-(1-methyl-1H-pyrazol-4- | ¹H NMR (400 MHz, DMSO- |
| 170 | yl)pyrazolo[1,5-a]pyridin-3-yl)piperazin- | d₆) δ (ppm) 8.84 (s, 1H), 8.37 |
| | 1-yl)pyrimidin-5-yl)methyl)oxazole | (s, 2H), 8.24 (s, 1H), 8.04 (s, |
| | | 1H), 7.98 (s, 1H), 7.79 (s, 1H), |
| | | 7.70 (d, J = 9.2 Hz, 1H), 7.40- |
| | | 7.27 (m, 1H), 7.13 (s, 1H), |
| | | 4.02 (s, 2H), 3.92 (t, J = 4.9 |
| | | Hz, 4H), 3.88 (s, 3H), 3.05 (t, |
| | | J = 5.0 Hz, 4H). MS (ESI) m/z |
| | | [M + H]⁺ calcd. for C₂₃H₂₃N₉O, |
| | | 442.2; found, 442.15; HPLC |
| | | purity: 254 nm: 99.7%. |
| Compound | 4-((2-(4-(6-(1-methyl-1H-pyrazol-4- | ¹H NMR (400 MHz, DMSO- |
| 171 | yl)pyrazolo[1,5-a]pyridin-3-yl)piperazin- | d₆) δ (ppm) 8.82 (s, 1H), 8.37- |
| | 1-yl)pyrimidin-5-yl)methyl)oxazole | 8.20 (m, 3H), 8.23 (s, 1H), |
| | | 7.97 (d, J = 0.8 Hz, 1H), 7.89- |
| | | 7.86 (m, 1H), 7.78 (s, 1H), |
| | | 7.74-7.64 (m, 1H), 7.33 (d, J = |
| | | 9.3 Hz, 1H), 3.90 (t, J = 5.1 |
| | | Hz, 4H), 3.87 (s, 3H), 3.70 (s, |
| | | 2H), 3.04 (t, J = 5.0 Hz, 4H). |
| | | MS (ESI) m/z [M + H]⁺ calcd. |
| | | for C₂₃H₂₃N₉O, 442.2; found, |
| | | 442.1; HPLC purity: 254 nm: |
| | | 98.7%. |
| Compound | 4-((2-(4-(6-(1-methyl-1H-pyrazol-4- | ¹H NMR (400 MHz, DMSO- |
| 172 | yl)pyrazolo[1,5-a]pyridin-3-yl)piperazin- | d₆) δ (ppm) 9.05 (d, J = 2.0 |
| | 1-yl)pyrimidin-5-yl)methyl)thiazole | Hz, 1H), 8.83 (s, 1H), 8.34 (s, |
| | | 2H), 8.24 (s, 1H), 7.97 (s, 1H), |
| | | 7.78 (s, 1H), 7.6 (d, J = 9.2, |
| | | Hz, 1H), 7.38 (d, J = 1.9 Hz, |
| | | 1H), 7.36-7.30 (m, 1H), 4.10- |
| | | 3.82(m, 9H), 3.05 (t, J = 5.0 |
| | | Hz, 4H). MS (ESI) m/z |
| | | [M + H]⁺ calcd. for C₂₃H₂₃N₉S, |
| | | 458.2; found, 458.2; HPLC |
| | | purity: 254 nm: 96.2%. |
| Compound | 3-[(2-{4-[6-(1-methylpyrazol-4- | ¹H NMR (400 MHz, DMSO- |
| 173 | yl)pyrazolo[1,5-a]pyridin-3-yl]piperazin- | d₆) δ (ppm) 10.87 (s, 1H), 8.82 |
| | 1-yl}pyrimidin-5-yl)methyl]-1H-indole | (s, 1H), 8.35 (s, 2H), 8.23 (s, |
| | | 1H), 7.97 (s, 1H), 7.77 (s, 1H), |
| | | 7.68 (d, J = 9.3 Hz, 1H), 7.49 |
| | | (d, J = 7.9 Hz, 1H), 7.38-7.29 |
| | | (m, 2H), 7.17 (d, J = 2.3 Hz, |
| | | 1H), 7.07 (t, J = 7.5 Hz, 1H), |
| | | 6.96 (t, J = 7.5 Hz, 1H), 3.92- |
| | | 3.85(m, 9H), 3.03 (t, J = 5.1 |
| | | Hz, 4H). MS (ESI) m/z |
| | | [M + H]⁺ calcd. for C₂₈H₂₇N₉, |
| | | 490.2; found, 490.2; HPLC |
| | | purity: 254 nm: 98.2%. |
| Compound | 3-(4-(5-(indolin-1-ylmethyl)pyrimidin-2- | ¹H NMR (400 MHz, DMSO- |
| 174 | yl)piperazin-1-yl)-6-(1-methyl-1H- | d₆) δ (ppm) 8.82 (s, 1H), 8.39 |
| | pyrazol-4-yl)pyrazolo[1,5-a]pyridine | (s, 2H), 8.22 (s, 1H), 7.97 (s, |
| | | 1H), 7.78 (s, 1H), 7.69 (d, J = |
| | | 9.2 Hz, 1H), 7.36-7.29 (m, |
| | | 1H), 7.02 (m, 2H), 6.71 (d, J = |
| | | 7.8 Hz, 1H), 6.59 (t, J = 7.3 |
| | | Hz, 1H), 4.14 (s, 2H), 3.95- |
| | | 3.85 (m, 7H), 3.21 (t, J = 8.3 |
| | | Hz, 2H), 3.04 (t, J = 5.0 Hz, |
| | | 4H), 2.85 (t, J = 8.2 Hz, 2H). |
| | | MS (ESI) m/z [M + H]⁺ calcd. |
| | | for C₂₈H₂₉N₉, 492.2; found, |
| | | 492.2; HPLC purity: 254 nm: |
| | | 96.2%. |

### Example 134: Synthesis of Exemplary Compound 175

### 1-(2-(4-(6-(1-methyl-1H-pyrazol-4-yl)pyrazolo[1,5-a]pyridin-3-yl)piperazin-1-yl)pyrimidin-5-yl)-3-phenylpropan-1-ol (Compound 175)

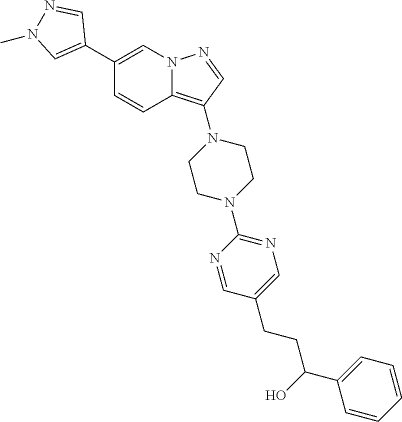 [SMILES: Cn1cc(-c2ccc3c(N4CCN(c5ncc(CCC(O)c6ccccc6)cn5)CC4)cnn3c2)cn1]

To a solution of 1-(2-chloropyrimidin-5-yl)-3-phenylpropan-1-ol (Compound S59) (100 mg, 0.40 mmol) and 1-[6-(1-methylpyrazol-4-yl)pyrazolo[1,5-a]pyridin-3-yl]piperazine (Compound 12) (100 mg, 0.35 mmol) in IPA (4 mL) was added DIPEA (155.9 mg, 1.21 mmol). The reaction was stirred for 16 h at 80° C. under nitrogen then allowed to cool to rt. The mixture was concentrated under reduced pressure then the residue was purified by Prep-HPLC to afford 75.7 mg (38%) of 1-(2-(4-(6-(1-methyl-1H-pyrazol-4-yl)pyrazolo[1,5-a]pyridin-3-yl)piperazin-1-yl)pyrimidin-5-yl)-3-phenylpropan-1-ol (Compound 175) as a light yellow solid. ¹H NMR (400 MHZ, DMSO) δ (ppm) 8.83 (s, 1H), 8.41-8.32 (m, 2H), 8.23 (s, 1H), 7.98 (s, 1H), 7.78 (s, 1H), 7.69 (d, J=9.2 Hz, 1H), 7.37-7.12 (m, 6H), 5.29 (s, 1H), 4.45 (t, J=6.6 Hz, 1H), 4.00-3.80 (m, 7H), 3.05 (t, J=5.1 Hz, 4H), 2.67-2.54 (m, 2H), 2.00-1.88 (m, 2H); MS (ESI) m/z [M+H]⁺ calcd. for C₂₆H₂₄N₈O, 495.3; found, 495.2; LCMS purity: 254 nm: 99.9%.

Using the procedures described in Example 134, Compound 175 and reagents, starting materials, and conditions known to those skilled in the art were used to prepare the compounds shown in Table U.

TABLE U

| Compound | Name | Characterization |
|---|---|---|
| Compound | 2-methyl-1-(2-{4-[6-(1-methyl-1H- | ¹H NMR (400 MHz, DMSO- |
| 176 | pyrazol-4-yl)pyrazolo[1,5-a]pyridin-3- | d₆) δ (ppm) 8.84 (t, J = 1.2 Hz, |
| | yl]piperazin-1-yl}pyrimidin-5-yl)propan- | 1H), 8.30 (s, 2H), 8.24 (s, 1H), |
| | 1-ol | 7.98 (d, J = 0.8 Hz, 1H), 7.79 |
| | | (s, 1H), 7.70 (dd, J = 9.3, 1.0 |
| | | Hz, 1H), 7.34 (dd, J = 9.3, 1.5 |
| | | Hz, 1H), 5.14 (d, J = 4.4 Hz, |
| | | 1H), 4.16 (dd, J = 6.6, 4.4 Hz, |
| | | 1H), 3.92 (t, J = 5.0 Hz, 4H), |
| | | 3.88 (s, 3H), 3.05 (t, J = 5.0 |
| | | Hz, 4H), 1.81-1.85 (m, 1H), |
| | | 0.89 (d, J = 6.6 Hz, 3H), 0.75 |
| | | (d, J = 6.6 Hz, 3H). MS (ESI) |
| | | m/z [M + H]⁺ calcd. for |
| | | C₂₃H₂₈N₈O, 433.2; found, |
| | | 433.2. HPLC Purity: 254nm: |
| | | 99.9%. |
| Compound | 2-ethyl-1-(2-(4-(6-(1-methyl-1H-pyrazol- | ¹H NMR (400 MHz, DMSO- |
| 177 | 4-yl)pyrazolo[1,5-a]pyridin-3- | d₆) δ (ppm) 8.83 (s, 1H), 8.31 |
| | yl)piperazin-1-yl)pyrimidin-5-yl)butan-1- | (s, 2H), 8.23 (s, 1H), 7.98 (s, |
| | ol | 1H), 7.79 (s, 1H), 7.70 (d, J = |
| | | 9.2 Hz, 1H), 7.34-7.32 (m, |
| | | 1H), 5.07 (d, J = 4.5 Hz, 1H), |
| | | 4.38-4.36 (m, 1H), 3.92 (t, J = |
| | | 4.9 Hz, 4H), 3.88 (s, 3H), |
| | | 3.06 (t, J = 4.9 Hz, 4H), 1.49- |
| | | 1.33 (m, 3H), 1.27-1.25 (m, |
| | | 1H), 1.08-1.05 (m, 1H), 0.83- |
| | | 0.80 (m, 6H). MS (ESI) m/z |
| | | [M + H]⁺ calcd. for C₂₅H₃₂N₈O, |
| | | 461.3; found, 461.3; HPLC |
| | | purity: 254 nm: 98.3%. |
| Compound | 6-(1-methyl-1H-pyrazol-4-yl)-3-(4-(5- | ¹H NMR (400 MHz, DMSO- |
| 178 | (thiophen-2-ylmethyl)pyrimidin-2- | d₆) δ (ppm) 8.83 (s, 1H), 8.34 |
| | yl)piperazin-1-yl)pyrazolo[1,5-a]pyridine | (s, 2H), 8.23 (s, 1H), 7.98 (s, |
| | | 1H), 7.78 (s, 1H), 7.69 (d, J = |
| | | 9.2 Hz, 1H), 7.39-7.30 (m, |
| | | 2H), 6.96 (m, 1H), 6.91 (m, |
| | | 1H), 4.02 (s, 2H), 3.92- |
| | | 3.91(m, 4H), 3.91-3.87 (m, |
| | | 3H), 3.05 (t, J = 5.0 Hz, 4H). |
| | | MS (ESI) m/z [M + H]⁺ calcd. |
| | | for C₂₄H₂₄N₈S, 457.2; found, |
| | | 457.2. HPLC purity: 254 nm: |
| | | 98.9%. |
| Compound | 6-(1-methyl-1H-pyrazol-4-yl)-3-(4-(5- | ¹H NMR (400 MHz, DMSO- |
| 179 | (pyridin-4-ylmethyl)pyrimidin-2- | d₆) δ (ppm) 8.83 (s, 1H), 8.55- |
| | yl)piperazin-1-yl)pyrazolo[1,5-a]pyridine | 8.43 (m, 2H), 8.35 (s, 2H), |
| | | 8.23 (s, 1H), 7.98 (s, 1H), 7.78 |
| | | (s, 1H), 7.69 (d, J = 9.2 Hz, |
| | | 1H), 7.38-7.31 (m, 1H), 7.27 |
| | | (d, J = 5.9 Hz, 2H), 3.91 (t, J = |
| | | 5.0 Hz, 4H), 3.87 (s, 3H), |
| | | 3.83 (s, 2H), 3.04 (t, J = 5.0 |
| | | Hz, 4H). MS (ESI) m/z |
| | | [M + H]⁺ calcd. for C₂₅H₂₅N₉, |
| | | 452.2; found, 452.20; HPLC |
| | | purity: 254 nm: 96.2%. |
| Compound | 2-((2-(4-(6-(1-methyl-1H-pyrazol-4- | ¹H NMR (400 MHz, DMSO- |
| 180 | yl)pyrazolo[1,5-a]pyridin-3-yl)piperazin- | d₆) δ (ppm) 9.16 (s, 1H), 8.83 |
| | 1-yl)pyrimidin-5-yl)methyl)-1,3,4- | (s, 1H), 8.40 (s, 2H), 8.23 (s, |
| | oxadiazole | 1H), 7.97 (s, 1H), 7.78 (s, 1H), |
| | | 7.69 (d, J = 9.6 Hz, 1H), 7.33 |
| | | (dd, J = 9.2, 1.6 Hz, 1H), 4.18 |
| | | (s, 2H), 3.93 (t, J = 4.8 Hz, |
| | | 4H), 3.87 (s, 3H), 3.05 (t, J = |
| | | 4.8 Hz, 4H). MS (ESI) m/z |
| | | [M + H]⁺ calcd. for |
| | | C₂₂H₂₂N₁₀O, 443.2; found, |
| | | 443.3. HPLC Purity: 254nm: |
| | | 98.5%. |

### Example 135: Synthesis of Exemplary Compound 181

### 3-(4-(5-(furan-2-ylmethyl)pyrimidin-2-yl)piperazin-1-yl)-6-(1-methyl-1H-pyrazol-4-yl)pyrazolo[1,5-a]pyridine (Compound 181)

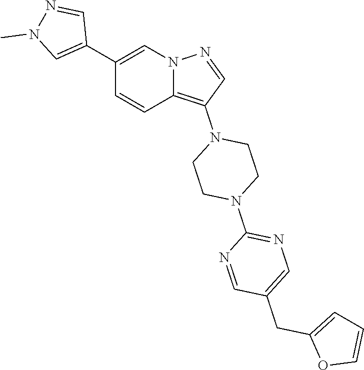 [SMILES: Cn1cc(-c2ccc3c(N4CCN(c5ncc(Cc6ccco6)cn5)CC4)cnn3c2)cn1]

To a solution of 2-chloro-5-(furan-2-ylmethyl)pyrimidine (Compound S64) (137.9 mg, 0.71 mmol) and 1-[6-(1-methylpyrazol-4-yl)pyrazolo[1,5-a]pyridin-3-yl]piperazine (Compound 12) (100 mg, 0.35 mmol) in IPA (5 mL) was added DIPEA (91.6 mg, 0.71 mmol). The reaction was stirred for 4 h at 100° C. under nitrogen then was allowed to cool to rt. The precipitated solids were collected by filtration and washed with EtOAc (3×10 mL). The residue was purified by trituration with EtOAc (10 mL) to afford 58.2 mg (37%) of 3-(4-(5-(furan-2-ylmethyl)pyrimidin-2-yl)piperazin-1-yl)-6-(1-methyl-1H-pyrazol-4-yl)pyrazolo[1,5-a]pyridine (Compound 181) as a grey white solid. ¹H NMR (400 MHZ, DMSO-d₆) δ (ppm) 8.87 (s, 1H), 8.34 (s, 2H), 8.27 (d, J=0.8 Hz, 1H), 8.01 (d, J=0.8 Hz, 1H), 7.82 (s, 1H), 7.73 (d, J=9.3 Hz, 1H), 7.58 (d, J=1.9 Hz, 1H), 7.37 (d, J=9.2 Hz, 1H), 6.40 (d, J=3.1 Hz, 1H), 6.17-6.12 (m, 1H), 3.95 (t, J=5.0 Hz, 4H), 3.91 (s, 3H), 3.87 (s, 2H), 3.08 (t, J=5.1 Hz, 4H). MS (ESI) m/z [M+H]⁺ calcd. for C₂₄H₂₄ClN₈O, 441.2; found, 441.1; HPLC purity: 254 nm: 99.7%.

### Example 136: Synthesis of Exemplary Compound 182

### 2-((2-(4-(6-(1-methyl-1H-pyrazol-4-yl)pyrazolo[1,5-a]pyridin-3-yl)piperazin-1-yl)pyrimidin-5-yl)methyl) thiazole (Compound 182)

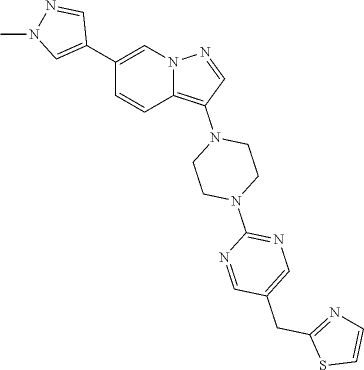 [SMILES: Cn1cc(-c2ccc3c(N4CCN(c5ncc(Cc6nccs6)cn5)CC4)cnn3c2)cn1]

To a solution of 1-[6-(1-methylpyrazol-4-yl)pyrazolo[1,5-a]pyridin-3-yl]piperazine (Compound 12) (107 mg, 0.38 mmol) and DIPEA (97.7 mg, 0.76 mmol) in IPA (2.0 mL) was added 2-((2-chloropyrimidin-5-yl)methyl) thiazole (Compound S72) (80.0 mg, 0.38 mmol). The reaction was stirred for 16 h at 120° C. then concentrated under reduced pressure. The residue was purified by trituration with MeOH (4.0 mL) to afford 121.9 mg (70%) of 2-((2-(4-(6-(1-methyl-1H-pyrazol-4-yl)pyrazolo[1,5-a]pyridin-3-yl)piperazin-1-yl)pyrimidin-5-yl)methyl) thiazole (Compound 182) as a brown solid. ¹H NMR (400 MHZ, DMSO-d₆) δ (ppm) 8.82 (t, J=1.2 Hz, 1H), 8.40 (s, 2H), 8.23 (s, 1H), 7.97 (d, J=0.8 Hz, 1H), 7.78 (s, 1H), 7.73 (d, J=3.3 Hz, 1H), 7.69 (dd, J=9.3, 1.0 Hz, 1H), 7.60 (d, J=3.3 Hz, 1H), 7.33 (dd, J=9.3, 1.5 Hz, 1H), 4.21 (s, 2H), 3.92 (t, J=5.0 Hz, 4H), 3.87 (s, 3H), 3.05 (t, J=5.0 Hz, 4H). MS (ESI) m/z [M+H]⁺ calcd. for C₂₂H₂₁N₉S, 458.2; found, 458.1; HPLC purity: 254 nm: 98.6%.

### Example 137: Synthesis of Exemplary Compound 183

### 6-(1-methyl-1H-pyrazol-4-yl)-3-(4-(5-(3-phenylpropyl)pyrimidin-2-yl)piperazin-1-yl)pyrazolo[1,5-a]pyridine (Compound 183)

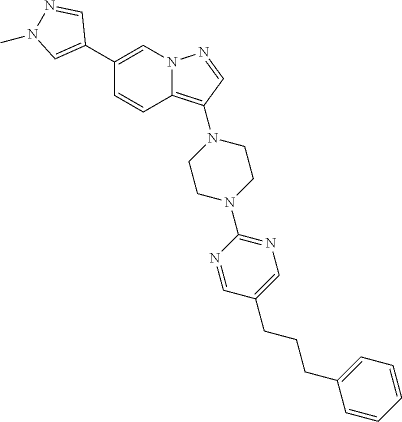 [SMILES: Cn1cc(-c2ccc3c(N4CCN(c5ncc(CCCc6ccccc6)cn5)CC4)cnn3c2)cn1]

To a solution of 1-(2-(4-(6-(1-methyl-1H-pyrazol-4-yl)pyrazolo[1,5-a]pyridin-3-yl)piperazin-1-yl)pyrimidin-5-yl)-3-phenylpropan-1-ol (Compound 175) (100 mg, 0.20 mmol) in DCM (3 mL) was added Et₃SiH (470.2 mg, 4.04 mmol) and TFA (922.1 mg, 8.08 mmol). The reaction was stirred for 16 h at 60° C. under nitrogen then allowed to cool to rt. The mixture was neutralized to pH 8 with aq. NaOH. The resulting mixture was extracted with DCM (3×50 mL). The combined organic layers were washed with brine (30 mL), dried over anhydrous Na₂SO₄. After filtration, the filtrate was concentrated under reduced pressure. The crude product was purified by Prep-HPLC to afford 44.3 mg (46%) of 6-(1-methyl-1H-pyrazol-4-yl)-3-(4-(5-(3-phenylpropyl)pyrimidin-2-yl)piperazin-1-yl)pyrazolo[1,5-a]pyridine (Compound 183) as a white solid. ¹H NMR (400 MHZ, DMSO-d₆) δ (ppm) 8.83 (s, 1H), 8.28 (s, 2H), 8.23 (s, 1H), 7.98 (d, J=0.8 Hz, 1H), 7.78 (s, 1H), 7.72-7.65 (m, 1H), 7.37-7.24 (m, 3H), 7.24-7.13 (m, 3H), 3.96-3.85 (m, 7H), 3.05 (t, J=5.0 Hz, 4H), 2.60 (t, J=7.7 Hz, 2H), 2.45 (t, J=7.6 Hz, 2H), 1.90-1.77 (m, 2H). MS (ESI) m/z [M+H]⁺ calcd. for C₂₈H₃₀N₈, 479.3; found, 479.3; HPLC purity: 254 nm: 99.5%.

Using the procedures described in Example 137, Compound 183 and reagents, starting materials, and conditions known to those skilled in the art were used to prepare the compounds shown in Table V.

TABLE V

| Compound | Name | Characterization |
|---|---|---|
| Compound | 6-(1-methyl-1H-pyrazol-4-yl)-3-(4-(5- | ¹H NMR (400 MHz, DMSO- |
| 184 | ((tetrahydro-2H-pyran-2- | d₆) δ (ppm) 8.86 (s, 1H), 8.23 |
| | yl)methyl)pyrimidin-2-yl)piperazin-1- | (d, J = 4.7 Hz, 3H), 7.97 (s, |
| | yl)pyrazolo[1,5-a]pyridine | 1H), 7.78 (s, 1H), 7.70-7.67 |
| | | (d, J= 9.3 Hz, 1H), 7.34-7.31 |
| | | (dd, J = 9.2, 1.5 Hz, 1H), 4.00- |
| | | 3.83 (m, 8H), 3.42-3.39 |
| | | (m, 1H), 3.33-3.26 (m, 1H), |
| | | 3.09 (t, J = 4.9 Hz, 4H), 2.59 |
| | | (d, J = 5.3 Hz, 2H), 1.80 (d, J = |
| | | 8.0 Hz, 1H), 1.61 (d, J = |
| | | 12.7 Hz, 1H), 1.46 (d, J = 5.2 |
| | | Hz, 3H), 1.28-1.17 (m, 1H). |
| | | MS (ESI) m/z [M + H]⁺ calcd. |
| | | for C₂₅H₃₀N₈O, 459.3; found, |
| | | 459.2; HPLC purity: 254 nm: |
| | | 99.0%. |
| Compound | 6-(1-methyl-1H-pyrazol-4-yl)-3-(4-(5- | ¹H NMR (400 MHz, DMSO- |
| 185 | ((tetrahydro-2H-pyran-3- | d₆) δ (ppm) 8.83 (s, 1H), 8.24 |
| | yl)methyl)pyrimidin-2-yl)piperazin-1- | (d, J = 4.6 Hz, 3H), 7.98 (s, |
| | yl)pyrazolo[1,5-a]pyridine | 1H), 7.79 (s, 1H), 7.70 (d, J = |
| | | 9.4 Hz, 1H), 7.33 (d, J = |
| | | 9.2Hz, 1H), 3.98-3.85 (m, |
| | | 7H), 3.72 (m, 2H), 3.31-3.23 |
| | | (m, 1H), 3.08-3.01 (m, 5H), |
| | | 2.39-2.16 (m, 2H), 1.69 (d, J = |
| | | 10.8 Hz, 2H), 1.61-1.53 (m, |
| | | 1H), 1.51-1.36 (m, 1H), 1.23- |
| | | 1.12 (m, 1H). MS (ESI) m/z |
| | | [M + H]⁺ calcd. for C₂₅H₃₀N₈O. |
| | | 459.5, found, 459.4. HPLC |
| | | purity: 254 nm: 99.2%. |

### Example 138: Synthesis of Exemplary Compound 186

### 6-(1-methyl-1H-pyrazol-4-yl)-3-(4-(5-(phenylsulfonyl)pyrimidin-2-yl)piperazin-1-yl)pyrazolo[1,5-a]pyridine (Compound 186)

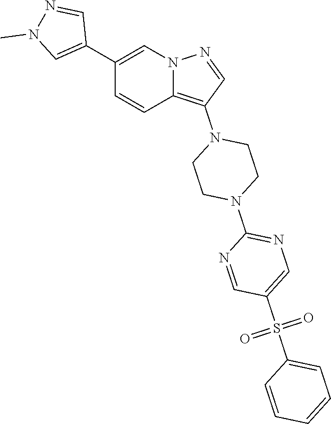 [SMILES: Cn1cc(-c2ccc3c(N4CCN(c5ncc(S(=O)(=O)c6ccccc6)cn5)CC4)cnn3c2)cn1]

To a solution of 6-(1-methyl-1H-pyrazol-4-yl)-3-(4-(5-(phenylthio)pyrimidin-2-yl)piperazin-1-yl)pyrazolo[1,5-a]pyridine (Compound 188) (200 mg, 0.43 mmol) in DCM (20 mL) was added mCPBA (368.3 mg, 2.14 mmol) at 0° C. The reaction was stirred for 2 h then quenched by the addition of sat. aq. Na₂SO₃ solution (2 mL) at 0° C. The resulting mixture was extracted with DCM (2×10 mL). The combined organic layers were concentrated under reduced pressure. The residue was purified by Prep-TLC (PE/EtOAc 1:1) and the crude product was purified by Prep-HPLC to afford 52.3 mg (25%) of 6-(1-methyl-1H-pyrazol-4-yl)-3-(4-(5-(phenylsulfonyl)pyrimidin-2-yl)piperazin-1-yl)pyrazolo[1,5-a]pyridine (Compound 186) as an off-white solid. ¹H NMR (400 MHZ, DMSO-d₆) δ (ppm) 8.87-8.81 (m, 3H), 8.23 (s, 1H), 8.04-7.95 (m, 3H), 7.77 (s, 1H), 7.75-7.67 (m, 2H), 7.67-7.59 (m, 2H), 7.34 (dd, J=9.3, 1.5 Hz, 1H), 4.05 (t, J=4.9 Hz, 4H), 3.87 (s, 3H), 3.06 (t, J=5.0 Hz, 4H). MS (ESI) m/z [M+H]⁺ calcd. for C₂₅H₂₄N₈O₂S, 501.1; found, 501.1; HPLC purity: 254 nm: 98.5%.

### Example 139: Synthesis of Exemplary Compound 187

### 6-(1-methyl-1H-pyrazol-4-yl)-3-(4-(5-(phenylsulfinyl)pyrimidin-2-yl)piperazin-1-yl)pyrazolo[1,5-a]pyridine (Compound 187)

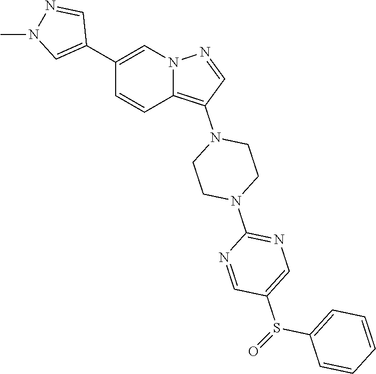 [SMILES: Cn1cc(-c2ccc3c(N4CCN(c5ncc(S(=O)c6ccccc6)cn5)CC4)cnn3c2)cn1]

To a solution of 6-(1-methyl-1H-pyrazol-4-yl)-3-(4-(5-(phenylthio)pyrimidin-2-yl)piperazin-1-yl)pyrazolo[1,5-a]pyridine (Compound 188) (250 mg, 0.53 mmol) dissolved in MeOH (20 mL) was added mCPBA (184.1 mg, 1.07 mmol). The reaction was stirred for 2 days at rt under nitrogen then neutralized to pH 8 with sat. aq. Na₂CO₃. The resulting mixture was extracted with EtOAc (3×20 mL). The combined organic layers were washed with brine (3×20 mL), dried over anhydrous Na₂SO₄, and filtered. The filtrate was concentrated under reduced pressure then the residue was purified by Prep-HPLC to afford 10.0 mg (4%) of 6-(1-methyl-1H-pyrazol-4-yl)-3-(4-(5-(phenylsulfinyl)pyrimidin-2-yl)piperazin-1-yl)pyrazolo[1,5-a]pyridine (Compound 187) as a white solid. ¹H NMR (400 MHZ, DMSO-d₆) δ (ppm) 8.83 (s, 1H), 8.58 (s, 2H), 8.23 (s, 1H), 7.97 (s, 1H), 7.79-7.65 (m, 4H), 7.63-7.50 (m, 3H), 7.33 (d, J=8.8 Hz, 1H), 4.05-3.95 (m, 4H), 3.93-3.84 (m, 3H), 3.05 (s, 4H). MS (ESI) m/z [M+H]⁺ calcd. for C₂₅H₂₄N₈OS, 485.2; found, 485.2; HPLC purity: 254 nm: 93.4%.

### Example 140: Synthesis of Exemplary Compound 188

### 6-(1-methyl-1H-pyrazol-4-yl)-3-(4-(5-(phenylthio)pyrimidin-2-yl)piperazin-1-yl)pyrazolo[1,5-a]pyridine (Compound 188)

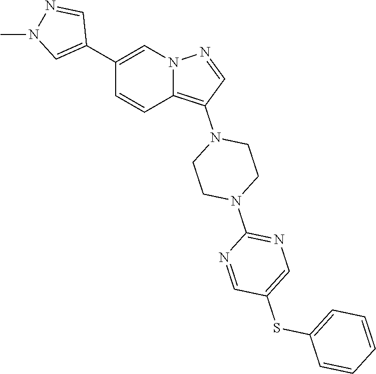 [SMILES: Cn1cc(-c2ccc3c(N4CCN(c5ncc(Sc6ccccc6)cn5)CC4)cnn3c2)cn1]

To a solution of 5-iodo-2-{4-[6-(1-methylpyrazol-4-yl)pyrazolo[1,5-a]pyridin-3-yl]piperazin-1-yl}pyrimidine (300 mg, 0.62 mmol, as prepared in Example 130, Step 1), KSPh (137.2 mg, 0.93 mmol), 1,10-phenanthroline (55.6 mg, 0.31 mmol) in DMF (10 mL) was added CuI (58.7 mg, 0.31 mmol) at rt. The reaction was stirred at 90° C. for 12 h, then allowed to cool to rt. The resulting mixture was diluted with H₂O (15 mL) and extracted with EtOAc (3×50 mL). The combined organic layers were dried over anhydrous Na₂SO₄. After filtration, the filtrate was concentrated under reduced pressure. The residue was purified by Prep-TLC (PE/EtOAc 1:5) and the crude product was purified by Prep-HPLC to afford 51 mg (18%) of 6-(1-methyl-1H-pyrazol-4-yl)-3-(4-(5-(phenylthio)pyrimidin-2-yl)piperazin-1-yl)pyrazolo[1,5-a]pyridine (Compound 188) as an off-white solid. ¹H NMR (400 MHZ, DMSO-d₆) δ (ppm) 8.84 (s, 1H), 8.53 (s, 2H), 8.23 (s, 1H), 7.98 (s, 1H), 7.80 (s, 1H), 7.71 (d, J=9.2 Hz, 1H), 7.39-7.28 (m, 3H), 7.24-7.14 (m, 3H), 4.05-3.96 (m, 4H), 3.87 (s, 3H), 3.13-3.04 (m, 4H). MS (ESI) m/z [M+H]⁺ calcd. for C₂₅H₂₄N₈S, 469.2; found, 469.2; HPLC purity: 254 nm: 99.3%.

### Example 141: Synthesis of Exemplary Compound 189

### Synthesis of 3-(4-(5-isobutylpyrimidin-2-yl)piperazin-1-yl)-6-(1-methyl-1H-pyrazol-4-yl)pyrazolo[1,5-a]pyridine (Compound 189)

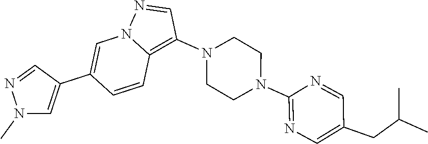 [SMILES: CC(C)Cc1cnc(N2CCN(c3cnn4cc(-c5cnn(C)c5)ccc34)CC2)nc1]

To a stirred solution of 2-methyl-1-(2-{4-[6-(1-methylpyrazol-4-yl)pyrazolo[1,5-a]pyridin-3-yl]piperazin-1-yl}pyrimidin-5-yl)propan-1-ol (Compound 176) (140 mg, 0.32 mmol) in DCM (20 mL) was added Et₃SiH (0.75 g, 6.48 mmol) and TFA (1.48 g, 12.98 mmol) dropwise at 0° C. under air atmosphere. The reaction was stirred overnight at 50° C. under air atmosphere then concentrated under reduced pressure. The mixture was dissolved in water (100 mL), then extracted with DCM (2×100 mL). The combined organic layers were washed with brine (2×5 mL) and dried over anhydrous Na₂SO₄. After filtration, the filtrate was concentrated under reduced pressure. The residue was purified by Prep-TLC (DCM/MeOH 10:1) to afford crude product. The crude product was purified by Prep-HPLC to afford 47.5 mg (35%) of 3-(4-(5-isobutylpyrimidin-2-yl)piperazin-1-yl)-6-(1-methyl-1H-pyrazol-4-yl)pyrazolo[1,5-a]pyridine (Compound 189) as a white solid. ¹H NMR (400 MHZ, DMSO-d₆) δ (ppm) 8.83 (s, 1H), 8.29-8.19 (m, 3H), 7.98 (s, 1H), 7.79 (d, J=2.2 Hz, 1H), 7.70 (d, J=9.2 Hz, 1H), 7.34 (d, J=9.3 Hz, 1H), 3.97-3.81 (m, 7H), 3.12-3.00 (m, 4H), 2.30 (d, J=7.0 Hz, 2H), 1.81-1.68 (m, 1H), 0.87 (d, J=6.7 Hz, 6H). MS (ESI) m/z [M+H]⁺ calcd. for C₂₃H₂₈N₈, 417.2; found, 417.2. HPLC Purity: 254 nm: 99.5%.

Using the procedures described in Example 141, Compound 189 and reagents, starting materials, and conditions known to those skilled in the art were used to prepare the compounds shown in Table W.

TABLE W

| Compound | Name | Characterization |
|---|---|---|
| Compound | 6-(1-methyl-1H-pyrazol-4-yl)-3-(4-(5- | ¹H NMR (400 MHz, DMSO- |
| 190 | pentylpyrimidin-2-yl)piperazin-1- | d₆) δ (ppm) 8.83 (s, 1H), 8.26 |
| | yl)pyrazolo[1,5-a]pyridine | (s, 2H), 8.23 (s, 1H), 7.97 (s, |
| | | 1H), 7.78 (s, 1H), 7.69 (d, J = |
| | | 9.2 Hz, 1H), 7.33 (dd, J = 9.2, |
| | | 0.8 Hz, 1H), 3.94-3.85 (m, |
| | | 7H), 3.04 (t, J = 4.8 Hz, 4H), |
| | | 2.41 (t, J = 7.6 Hz, 2H), 1.58- |
| | | 1.45 (m, 2H), 1.38-1.19 (m, |
| | | 4H), 0.87 (t, J = 6.8 Hz, 3H). |
| | | MS (ESI) m/z [M + H]⁺ calcd. |
| | | for C₂₄H₃₀N₈, 431.3; found, |
| | | 431.2; HPLC purity: 254 nm: |
| | | 99.1%. |

### Example 142: Synthesis of Exemplary Compound 191

### 3-(4-(5-((1H-indol-1-yl)methyl)pyrimidin-2-yl)piperazin-1-yl)-6-(1-methyl-1H-pyrazol-4-yl)pyrazolo[1,5-a]pyridine (Compound 191)

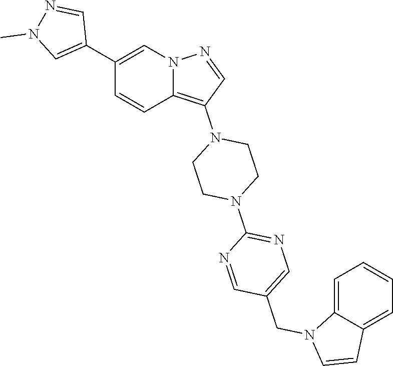 [SMILES: Cn1cc(-c2ccc3c(N4CCN(c5ncc(Cn6ccc7ccccc76)cn5)CC4)cnn3c2)cn1]

To a solution of 1-[(2-{4-[6-(1-methylpyrazol-4-yl)pyrazolo[1,5-a]pyridin-3-yl]piperazin-1-yl}pyrimidin-5-yl)methyl]-2,3-dihydroindole (Compound 174) (200 mg, 0.41 mmol) in DCM (20 mL) was added MnO₂ (353.7 mg, 4.07 mmol). The reaction was stirred for 2 h at rt under nitrogen then concentrated under reduced pressure. The residue was purified by silica gel column chromatography eluting with DCM/MeOH (10:1) to afford the crude product. The crude product (100 mg) was purified by Prep-HPLC to afford 32.7 mg (16%) of 3-(4-(5-((1H-indol-1-yl)methyl)pyrimidin-2-yl)piperazin-1-yl)-6-(1-methyl-1H-pyrazol-4-yl)pyrazolo[1,5-a]pyridine (Compound 191) as a grey-white solid. ¹H NMR (400 MHZ, DMSO-d₆) δ (ppm) 8.82 (s, 1H), 8.43 (s, 2H), 8.22 (s, 1H), 7.97 (s, 1H), 7.75 (s, 1H), 7.67 (d, J=9.3 Hz, 1H), 7.61 (d, J=8.2 Hz, 1H), 7.58-7.52 (m, 2H), 7.32 (d, J=9.3 Hz, 1H), 7.14 (t, J=7.6 Hz, 1H), 7.03 (t, J=7.4 Hz, 1H), 6.47 (d, J=3.1 Hz, 1H), 5.28 (s, 2H), 3.88 (d, J=5.8 Hz, 7H), 3.01 (t, J=4.9 Hz, 4H). MS (ESI) m/z [M+H]⁺ calcd. for C₂₈H₂₇N₉ 490.2, found, 490.2. HPLC purity: 254 nm: 98.7%.

### Example 143: Synthesis of Exemplary Compound 192 and Compound 193

### (Racemic) (R)-(2-(4-(6-(1-methyl-1H-pyrazol-4-yl)pyrazolo[1,5-a]pyridin-3-yl)piperazin-1-yl)pyrimidin-5-yl)((R)-tetrahydro-2H-pyran-3-yl)methanol (Compound 192) and (Racemic) (S)-(2-(4-(6-(1-methyl-1H-pyrazol-4-yl)pyrazolo[1,5-a]pyridin-3-yl)piperazin-1-yl)pyrimidin-5-yl)((R)-tetrahydro-2H-pyran-3-yl)methanol (Compound 193)

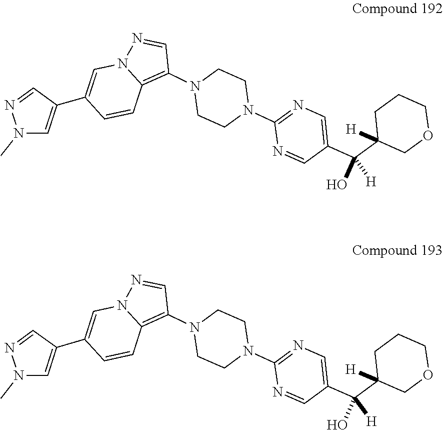 [SMILES: Cn1cc(-c2ccc3c(N4CCN(c5ncc([C@H](O)[C@@H]6CCCOC6)cn5)CC4)cnn3c2)cn1 ; Cn1cc(-c2ccc3c(N4CCN(c5ncc([C@@H](O)[C@@H]6CCCOC6)cn5)CC4)cnn3c2)cn1]

### Step 1. Preparation of (2-(4-(6-(1-methyl-1H-pyrazol-4-yl)pyrazolo[1,5-a]pyridin-3-yl)piperazin-1-yl)pyrimidin-5-yl) magnesium iodide

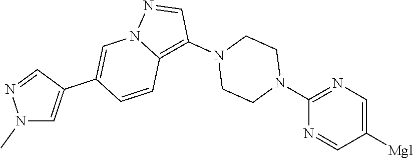

To a stirred solution of 5-iodo-2-{4-[6-(1-methylpyrazol-4-yl)pyrazolo[1,5-a]pyridin-3-yl]piperazin-1-yl}pyrimidine (200 mg, 0.41 mmol, as prepared in Example 130, Step 1) in THF (15 mL) was added 1M iPrMgBr (4.1 ml, 0.41 mmol) dropwise at 0° C. under nitrogen. The reaction was stirred for 1 h at rt under nitrogen then used in the next step without further purification.

### Step 2. (Racemic) (R)-(2-(4-(6-(1-methyl-1H-pyrazol-4-yl)pyrazolo[1,5-a]pyridin-3-yl)piperazin-1-yl)pyrimidin-5-yl)((R)-tetrahydro-2H-pyran-3-yl)methanol (Compound 192) and (Racemic) (S)-(2-(4-(6-(1-methyl-1H-pyrazol-4-yl)pyrazolo[1,5-a]pyridin-3-yl)piperazin-1-yl)pyrimidin-5-yl)((R)-tetrahydro-2H-pyran-3-yl)methanol (Compound 193)

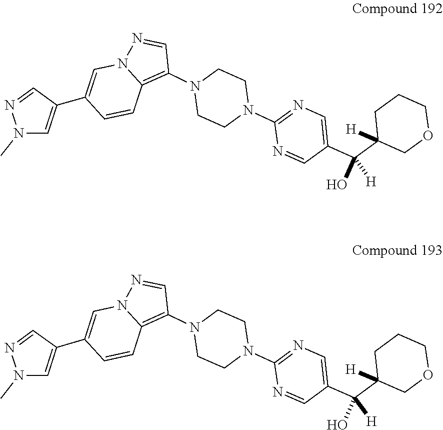 [SMILES: Cn1cc(-c2ccc3c(N4CCN(c5ncc([C@H](O)[C@@H]6CCCOC6)cn5)CC4)cnn3c2)cn1 ; Cn1cc(-c2ccc3c(N4CCN(c5ncc([C@@H](O)[C@@H]6CCCOC6)cn5)CC4)cnn3c2)cn1]

To the 5-(iodomagnesio)-2-{4-[6-(1-methylpyrazol-4-yl)pyrazolo[1,5-a]pyridin-3-yl]piperazin-1-yl}pyrimidine (100 mg, 0.2 mmol, as prepared in the previous step) mixture was added tetrahydro-2H-pyran-3-carbaldehyde (22.4 mg, 0.2 mmol) dropwise over 1 min at rt. The reaction was stirred for 1 h at rt then concentrated under reduced pressure. The residue was dissolved in water (50 mL), extracted with EtOAc (3×100 mL), and dried over anhydrous Na₂SO₄. After filtration, the filtrate was concentrated under reduced pressure. The crude product (100 mg) was purified by Prep-HPLC to afford 14.6 mg (15%) of (racemic) (R)-(2-(4-(6-(1-methyl-1H-pyrazol-4-yl)pyrazolo[1,5-a]pyridin-3-yl)piperazin-1-yl)pyrimidin-5-yl) ((R)-tetrahydro-2H-pyran-3-yl)methanol (Compound 192) as a white solid and 19.5 mg (20%) of (racemic) (S)-(2-(4-(6-(1-methyl-1H-pyrazol-4-yl)pyrazolo[1,5-a]pyridin-3-yl)piperazin-1-yl)pyrimidin-5-yl)((R)-tetrahydro-2H-pyran-3-yl)methanol (Compound 193) as a grey solid.

(racemic) (R)-(2-(4-(6-(1-methyl-1H-pyrazol-4-yl)pyrazolo[1,5-a]pyridin-3-yl)piperazin-1-yl)pyrimidin-5-yl)((R)-tetrahydro-2H-pyran-3-yl)methanol (Compound 192): ¹H NMR (400 MHZ, DMSO-d₆) δ (ppm) 8.83 (s, 1H), 8.30 (s, 2H), 8.24 (s, 1H), 7.98 (s, 1H), 7.79 (s, 1H), 7.70 (d, J=9.2 Hz, 1H), 7.34 (d, J=9.3 Hz, 1H), 5.22 (d, J=4.6 Hz, 1H), 4.35-4.20 (m, 1H), 3.93 (t, J=4.9 Hz, 4H), 3.88 (s, 3H), 3.73 (d, J=11.2 Hz, 1H), 3.59-3.51 (m, 1H), 3.28-3.19 (m, 1H), 3.14-3.02 (m, 5H), 1.92-1.68 (d, J=7.0 Hz, 1H), 1.62-1.52 (m, 1H), 1.52-1.33 (m, 2H). MS (ESI) m/z [M+H]⁺ calcd. for C₂₅H₃₀N₈O₂, 475.2; found, 475.2. HPLC purity: 254 nm: 97.9%.

(racemic) (S)-(2-(4-(6-(1-methyl-1H-pyrazol-4-yl)pyrazolo[1,5-a]pyridin-3-yl)piperazin-1-yl)pyrimidin-5-yl)((R)-tetrahydro-2H-pyran-3-yl)methanol (Compound 193): ¹H NMR (400 MHZ, DMSO-d₆) δ (ppm) 8.83 (s, 1H), 8.32 (s, 2H), 8.23 (s, 1H), 7.97 (s, 1H), 7.78 (s, 1H), 7.69 (d, J=9.3 Hz, 1H), 7.33 (dd, J=9.3 Hz, 1.2 Hz, 1H), 5.21 (d, J=4.6 Hz, 1H), 4.19-4.13 (m, 1H), 4.09-4.01 (m, 1H), 3.93 (t, J=5.0 Hz, 4H), 3.88 (s, 3H), 3.73 (d, J=11.2 Hz, 1H), 3.30-3.17 (m, 2H), 3.06 (t, J=5.0 Hz, 4H), 1.84-1.71 (m, 1H), 1.53-1.28 (m, 3H), 1.16-1.01 (m, 1H). MS (ESI) m/z [M+H]⁺ calcd. for C₂₅H₃₀N₈O₂, 475.2; found, 475.2. HPLC purity: 254 nm: 97.4%.

Using the procedures described in Example 143, Compound 192 and reagents, starting materials, and conditions known to those skilled in the art were used to prepare the compounds shown in Table X.

TABLE X

| Compound | Name | Characterization |
|---|---|---|
| Compound | (2-(4-(6-(1-methyl-1H-pyrazol-4- | ¹H NMR (400 MHz, DMSO- |
| 194 | yl)pyrazolo[1,5-a]pyridin-3-yl)piperazin- | d₆) δ (ppm) 8.86 (s, 1H), 8.34 |
| | 1-yl)pyrimidin-5-yl)(tetrahydro-2H-pyran- | (s, 2H), 8.27 (s, 1H), 8.01 (d, J = |
| | 2-yl)methanol | 0.8 Hz, 1H), 7.82 (s, 1H), |
| | | 7.73 (d, J = 9.3 Hz, 1H), 7.37 |
| | | (d, J = 9.3 Hz, 1H), 5.27 (d, J = |
| | | 4.6 Hz, 1H), 4.41 (t, J = 4.9 |
| | | Hz, 1H), 3.99-3.89 (m, 8H), |
| | | 3.39 (s, 2H), 3.09 (t, J = 5.0 |
| | | Hz, 4H), 1.85-1.70 (m, 1H), |
| | | 1.46-1.35 (m, 4H), 1.20 (m, |
| | | 1H). MS (ESI) m/z [M + H]⁺ |
| | | calcd. for C₂₅H₃₀N₈O₂, 475.2; |
| | | found, 475.2; HPLC purity: |
| | | 254 nm: 98.8%. |

### Example 144: Synthesis of Exemplary Compound 195

### (5-chlorothiazol-2-yl)methyl 4-(6-(1-methyl-1H-pyrazol-4-yl)pyrazolo[1,5-a]pyridin-3-yl)piperazine-1-carboxylate (Compound 195)

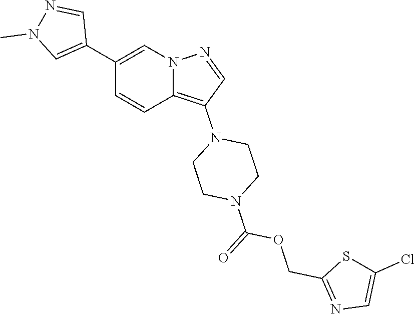 [SMILES: Cn1cc(-c2ccc3c(N4CCN(C(=O)OCc5ncc(Cl)s5)CC4)cnn3c2)cn1]

Cs₂CO₃ (1.73 g, 5.31 mmol) was dissolved in DMF (20 mL) and stirred at 0° C. under a carbon dioxide atmosphere. To the solution was added TBAI (1.18 g, 3.19 mmol) and 1-[6-(1-methylpyrazol-4-yl)pyrazolo[1,5-a]pyridin-3-yl]piperazine (Compound 12) (300 mg, 1.06 mmol). The reaction was stirred for 4 h at 0° C. under carbon dioxide atmosphere. 5-chloro-2-(chloromethyl)-1,3-thiazole (357 mg, 2.13 mmol) was added to the solution. The reaction was stirred for 60 h at rt under carbon dioxide atmosphere. Water (40 mL) was added, and the mixture was extracted with EtOAc (3×40 mL). The combined organic layers were dried over anhydrous Na₂SO₄. After filtration, the filtrate was concentrated under reduced pressure. The residue was purified by silica gel column chromatography eluting with DCM/MeOH (12:1). The crude product was purified by Prep-HPLC to afford 76.6 mg (16%) of (5-chlorothiazol-2-yl)methyl 4-(6-(1-methyl-1H-pyrazol-4-yl)pyrazolo[1,5-a]pyridin-3-yl)piperazine-1-carboxylate (Compound 195) as a white solid. ¹H NMR (400 MHZ, DMSO-d₆) δ (ppm) 8.83 (t, J=1.3 Hz, 1H), 8.23 (s, 1H), 7.98 (d, J=0.8 Hz, 1H), 7.77 (s, 1H), 7.76 (s, 1H), 7.65 (dd, J=9.3, 1.0 Hz, 1H), 7.33 (dd, J=9.3, 1.5 Hz, 1H), 5.28 (s, 2H), 3.87 (s, 3H), 3.57 (t, J=5.0 Hz, 4H), 2.99-2.94 (m, 4H). MS (ESI) m/z [M+H]⁺ calcd. for C₂₀H₂₀ClN₇O₂S, 458.1; found, 458.0; HPLC purity: 254 nm: 99.9%.

Using the procedures described in Example 144, Compound 195 and reagents, starting materials, and conditions known to those skilled in the art were used to prepare the compounds shown in Table Y.

TABLE Y

| Compound | Name | Characterization |
|---|---|---|
| Compound | (5-chlorothiophen-2-yl)methyl 4-(6-(1- | ¹H NMR (400 MHz, DMSO- |
| 196 | methyl-1H-pyrazol-4-yl)pyrazolo[1,5- | d₆) δ (ppm) 8.83 (s, 1H), 8.23 |
| | a]pyridin-3-yl)piperazine-1-carboxylate | (s, 1H), 7.97 (s, 1H), 7.76 (s, |
| | | 1H), 7.64 (d, J = 9.2 Hz, 1H), |
| | | 7.33 (d, J = 9.2 Hz, 1H), 7.10- |
| | | 7.01 (m, 2H), 5.20 (s, 2H), |
| | | 3.87 (s, 3H), 3.57 (t, J = 4.8 |
| | | Hz, 4H), 3.09-2.89 (m, 4H). |
| | | MS (ESI) m/z [M + H]⁺ calcd. |
| | | for C₂₁H₂₁ClN₆O₂S, 457.1; |
| | | found, 457.1; HPLC purity: |
| | | 254 nm: 99.8%. |
| Compound | (5-chlorothiophen-3-yl)methyl 4-(6-(1- | ¹H NMR (400 MHz, DMSO- |
| 197 | methyl-1H-pyrazol-4-yl)pyrazolo[1,5- | d₆) δ (ppm) 8.83 (s, 1H), 8.22 |
| | a]pyridin-3-yl)piperazine-1-carboxylate | (s, 1H), 7.97 (s, 1H), 7.76 (s, |
| | | 1H), 7.68-7.61 (m, 1H), 7.47- |
| | | 7.41 (m, 1H), 7.37-7.30 |
| | | (m, 1H), 7.15 (d, J = 1.6 Hz, |
| | | 1H), 5.01 (s, 2H), 3.86 (s, 3H), |
| | | 3.63-3.52 (m, 4H), 3.03- |
| | | 2.91 (m, 4H). MS (ESI) m/z |
| | | [M + H]⁺ calcd. for |
| | | C₂₁H₂₁ClN₆O₂S, 457.1; found, |
| | | 457.1; HPLC purity: 254 nm: |
| | | 99.6%. |
| Compound | 4-chlorobenzyl 4-(6-(1-methyl-1H- | ¹H NMR (400 MHz, DMSO- |
| 198 | pyrazol-4-yl)pyrazolo[1,5-a]pyridin-3- | d₆) δ (ppm) 8.83 (s, 1H), 8.23 |
| | yl)piperazine-1-carboxylate | (s, 1H), 7.97 (s, 1H), 7.77 (s, |
| | | 1H), 7.65 (dd, J = 9.3, 1.0 Hz, |
| | | 1H), 7.49-7.39 (m, 4H), 7.33 |
| | | (dd, J = 9.2, 1.5 Hz, 1H), 5.11 |
| | | (s, 2H), 3.87 (s, 3H), 3.60 (s, |
| | | 4H), 2.97 (t, J = 5.0 Hz, 4H). |
| | | MS (ESI) m/z [M + H]⁺ calcd. |
| | | for C₂₃H₂₃ClN₆O₂, 451.2; |
| | | found, 451.1; HPLC purity: |
| | | 254 nm: 98.5%. |
| Compound | (5-methyloxazol-2-yl)methyl 4-(6-(1- | ¹H NMR (400 MHz, DMSO- |
| 199 | methyl-1H-pyrazol-4-yl)pyrazolo[1,5- | d₆) δ (ppm) 8.83 (s, 1H), 8.23 |
| | a]pyridin-3-yl)piperazine-1-carboxylate | (s, 1H), 7.97 (d, J = 0.8 Hz, |
| | | 1H), 7.77 (s, 1H), 7.65 (dd, J = |
| | | 9.3, 1.0 Hz, 1H), 7.33 (dd, J = |
| | | 9.2, 1.5 Hz, 1H), 6.86 (d, J = |
| | | 0.9 Hz, 1H), 5.12 (s, 2H), |
| | | 3.87 (s, 3H), 3.58 (t, J = 5.0 |
| | | Hz, 4H), 2.98 (t, J = 4.8 Hz, |
| | | 4H), 2.31 (d, J = 1.3 Hz, 3H). |
| | | MS (ESI) m/z [M + H]⁺ calcd. |
| | | for C₂₁H₂₃N₇O₃, 422.2; found, |
| | | 422.2; HPLC purity: 254 nm: |
| | | 98.6%. |

### Example 145: Synthesis of Exemplary Compound 200

### 3-(4-((5-chlorothiophen-3-yl)methyl)piperazin-1-yl)-6-(1-methyl-1H-pyrazol-4-yl)pyrazolo[1,5-a]pyridine (Compound 200)

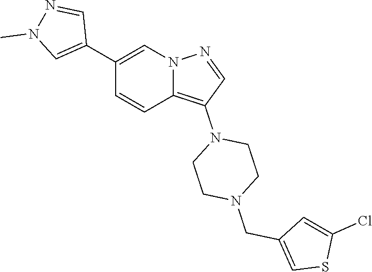 [SMILES: Cn1cc(-c2ccc3c(N4CCN(Cc5csc(Cl)c5)CC4)cnn3c2)cn1]

To a solution of 1-[6-(1-methylpyrazol-4-yl)pyrazolo[1,5-a]pyridin-3-yl]piperazine (Compound 12) (300 mg, 1.06 mmol) in DMF (20 mL) were added TBAI (784.9 mg, 2.13 mmol), Cs₂CO₃ (161.8 mg, 2.13 mmol), and 2-chloro-4-(chloromethyl)thiophene (Compound S73) (355 mg, 2.13 mmol). The reaction was stirred overnight at rt under carbon dioxide atmosphere then concentrated under reduced pressure. The residue was dissolved in water (100 mL), extracted with EtOAc (3×100 mL) and the combined organic extracts were dried over anhydrous Na₂SO₄. After filtration, the filtrate was concentrated under reduced pressure. The crude product was purified by Prep-HPLC to afford 44.5 mg (10%) of 3-(4-((5-chlorothiophen-3-yl)methyl)piperazin-1-yl)-6-(1-methyl-1H-pyrazol-4-yl)pyrazolo[1,5-a]pyridine (Compound 200) as a white solid. ¹H NMR (400 MHZ, DMSO-d₆) δ (ppm) 8.79 (s, 1H), 8.22 (s, 1H), 7.96 (d, J=0.8 Hz, 1H), 7.73 (s, 1H), 7.61 (d, J=9.3 Hz, 1H), 7.37-7.25 (m, 2H), 7.06 (d, J=1.6 Hz, 1H), 3.87 (s, 3H), 3.49 (s, 2H), 3.01 (t, J=5.1 Hz, 4H), 2.56 (t, J=5.1 Hz, 4H). MS (ESI) m/z [M+H]⁺ calcd. for C₂₀H₂₁ClN₆S, 415.1; found, 415.0; HPLC purity: 254 nm: 99.5%.

Using the procedures described in Example 145, Compound 200 and reagents, starting materials, and conditions known to those skilled in the art were used to prepare the compounds shown in Table Z.

TABLE Z

| Compound | Name | Characterization |
|---|---|---|
| Compound | 6-(1-methyl-1H-pyrazol-4-yl)-3-(4-((5- | ¹H NMR (400 MHz, DMSO- |
| 201 | methylfuran-2-yl)methyl)piperazin-1- | d₆) δ (ppm) 8.79 (s, 1H), 8.21 |
| | yl)pyrazolo[1,5-a]pyridine | (s, 1H), 7.95 (s, 1H), 7.71 (s, |
| | | 1H), 7.61-7.59 (d, J = 9.2 Hz, |
| | | 1H), 7.29-7.26 (dd, J = 9.3, |
| | | 1.6 Hz, 1H), 6.17 (s, 1H), 6.01 |
| | | (d, J = 3.0 Hz, 1H), 3.87 (s, |
| | | 3H), 3.50 (s, 2H), 3.01 (s, 4H), |
| | | 2.64-2.54 (m, 4H), 2.26 (s, |
| | | 3H). MS (ESI) m/z [M + H]⁺ |
| | | calcd. for C₂₁H₂₄N₆O, 377.2; |
| | | found, 377.2; HPLC purity: |
| | | 254 nm: 98.9%. |
| Compound | 3-(4-(4-chlorobenzyl)piperazin-1-yl)-6-(1- | ¹H NMR (400 MHz, DMSO- |
| 202 | methyl-1H-pyrazol-4-yl)pyrazolo[1,5- | d₆) δ (ppm) 8.80 (s, 1H), 8.22 |
| | a]pyridine | (s, 1H), 7.96 (s, 1H), 7.73 (s, |
| | | 1H), 7.61 (d, J = 9.2 Hz, 1H), |
| | | 7.44-7.34 (m, 4H), 7.29 (dd, |
| | | J = 9.2, 1.5 Hz, 1H), 3.87 (s, |
| | | 3H), 3.55 (s, 2H), 3.02 (t, J = |
| | | 4.8 Hz, 4H), 2.56 (t, J = 4.7 |
| | | Hz, 4H). MS (ESI) m/z |
| | | [M + H]⁺ calcd. for |
| | | C₂₂H₂₃ClN₆, 407.2; found, |
| | | 407.1; HPLC purity: 254 nm: |
| | | 99.2%. |

### Example 146: Synthesis of Exemplary Compound 203

### tert-butyl 4-(6-propylpyrazolo[1,5-a]pyridin-3-yl)piperazine-1-carboxylate (Compound 203)

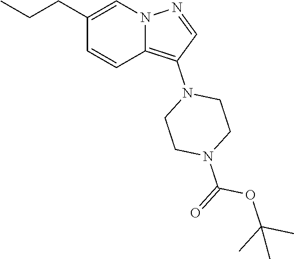 [SMILES: CCCc1ccc2c(N3CCN(C(=O)OC(C)(C)C)CC3)cnn2c1]

### Step 1. Preparation of (E)-6-(prop-1-en-1-yl)pyrazolo[1,5-a]pyridine

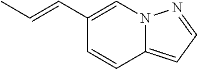

To a stirred solution of 6-bromopyrazolo[1,5-a]pyridine (3.5 g, 17.8 mmol) and 4,4,5,5-tetramethyl-2-(prop-1-en-1-yl)-1,3,2-dioxaborolane (3.3 g, 19.5 mmol) in dioxane (60.0 mL) and H₂O (6.0 mL) were added Pd(dppf)Cl₂·DCM (2.2 g, 2.66 mmol) and K₂CO₃ (4.9 g, 35.5 mmol) at rt. The reaction was stirred for 4 h at 90° C. under nitrogen. The resulting mixture was filtered, and the filter cake was washed with DCM (3×100 mL). The filtrate was concentrated under reduced pressure. The residue was purified by silica gel column chromatography eluting with PE/EtOAc (2:1) to afford 1.25 g (45%) of 6-(prop-1-en-1-yl)pyrazolo[1,5-a]pyridine as a green solid. ¹H NMR (400 MHZ, DMSO-d₆) δ (ppm) 8.62 (s, 1H), 7.98-7.92 (m, 1H), 7.63 (d, J=9.1 Hz, 1H), 7.49-7.40 (m, 1H), 6.57 (s, 1H), 6.46-6.20 (m, 2H), 1.87 (d, J=6.1 Hz, 3H). MS (ESI) m/z [M+H]⁺ calcd. for C₁₀H₁₀N₂, 159.1; found, 159.2.

### Step 2. Preparation of 6-propylpyrazolo[1,5-a]pyridine

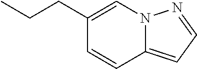

Into a 100 mL round-bottom flask was added 6-[(1E)-prop-1-en-1-yl]pyrazolo[1,5-a]pyridine (1.25 g, 7.90 mmol, as prepared in the previous step), MeOH (15.0 mL) and Pd/C (0.42 g, 3.95 mmol). The reaction was stirred for 2 h at rt under hydrogen atmosphere. The resulting mixture was filtered, and the filter cake was washed with MeOH (2×5 mL). The filtrate was concentrated under reduced pressure to afford 1.11 g (79%) of 6-propylpyrazolo[1,5-a]pyridine as a green liquid. ¹H NMR (400 MHZ, DMSO-d₆) δ (ppm) 8.49 (s, 1H), 7.92 (t, J=1.7 Hz, 1H), 7.61 (dd, J=9.0, 1.1 Hz, 1H), 7.15-7.07 (m, 1H), 6.57-6.51 (m, 1H), 2.56 (t, J=7.5 Hz, 2H), 1.69-1.55 (m, 2H), 0.95-0.85 (m, 3H). MS (ESI) m/z [M+H]⁺ calcd. for C₁₀H₁₂N₂, 161.1; found, 161.2.

### Step 3. Preparation of 3-bromo-6-propylpyrazolo[1,5-a]pyridine

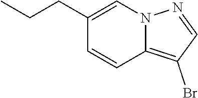

To a solution of 6-propylpyrazolo[1,5-a]pyridine (1.1 g, 6.87 mmol, as prepared in the previous step) in DCM (20.0 mL) was added NBS (1.34 g, 7.53 mmol). The reaction was stirred for 2 h at rt then concentrated under reduced pressure. The residue was purified by silica gel column chromatography eluting with PE/EtOAc (9:1) to afford 1.44 g (79%) of 3-bromo-6-propylpyrazolo[1,5-a]pyridine as a yellow liquid. ¹H NMR (400 MHZ, DMSO-d₆) δ (ppm) 8.56 (d, J=4.3 Hz, 1H), 8.07 (d, J=4.2 Hz, 1H), 7.50 (dd, J=9.4, 4.2 Hz, 1H), 7.25 (t, J=6.6 Hz, 1H), 2.58 (t, J=7.4 Hz, 2H), 1.70-1.54 (m, 2H), 0.97-0.83 (m, 3H). MS (ESI) m/z [M+H]⁺ calcd. for C₁₀H₁₁BrN₂, 239.0; found, 239.1.

### Step 4. Preparation of tert-butyl 4-(6-propylpyrazolo[1,5-a]pyridin-3-yl)piperazine-1-carboxylate (Compound 203)

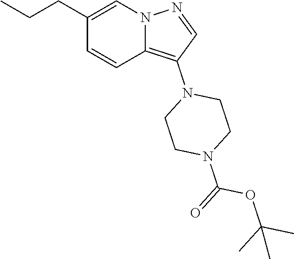 [SMILES: CCCc1ccc2c(N3CCN(C(=O)OC(C)(C)C)CC3)cnn2c1]

To a solution of 3-bromo-6-propylpyrazolo[1,5-a]pyridine (200 mg, 0.84 mmol, as prepared in the previous step) and tert-butyl piperazine-1-carboxylate (186.9 mg, 1.00 mmol) in tBuOH (8.0 mL) and dioxane (4.0 mL) was added KOtBu (140.8 mg, 1.26 mmol) and tBuXPhos Pd G1 (86.2 mg, 0.13 mmol) under nitrogen. The reaction was stirred for 16 h at 80° C. under nitrogen then concentrated under reduced pressure. The residue was purified by silica gel column chromatography eluting with PE/EtOAc (5:1). The crude product was purified by Prep-HPLC to afford 82.0 mg (28%) tert-butyl 4-(6-propylpyrazolo[1,5-a]pyridin-3-yl)piperazine-1-carboxylate (Compound 203) as a white solid. ¹H NMR (400 MHZ, DMSO-d₆) δ (ppm) 8.33 (t, J=1.2 Hz, 1H), 7.72 (s, 1H), 7.55 (dd, J=9.1, 0.9 Hz, 1H), 6.98 (dd, J=9.1, 1.5 Hz, 1H), 3.49 (t, J=4.9 Hz, 4H), 2.91 (dd, J=5.9, 4.2 Hz, 4H), 2.54 (d, J=7.5 Hz, 2H), 1.68-1.54 (m, 2H), 1.42 (s, 9H), 0.90 (t, J=7.3 Hz, 3H). MS (ESI) m/z [M+H]⁺ calcd. for C₁₉H₂₈N₄O₂, 345.2; found, 345.2; HPLC purity: 254 nm: 99.4%.

### Example 147: Synthesis of Exemplary Compound 204

### tert-butyl 4-(6-(3-hydroxypropyl)pyrazolo[1,5-a]pyridin-3-yl)piperazine-1-carboxylate (Compound 204)

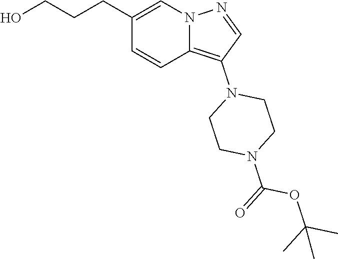 [SMILES: CC(C)(C)OC(=O)N1CCN(c2cnn3cc(CCCO)ccc23)CC1]

### Step 1. Preparation of (E)-6-(3-((tert-butyldimethylsilyl)oxy)prop-1-en-1-yl)pyrazolo[1,5-a]pyridine

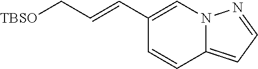 [SMILES: CC(C)(C)[Si](C)(C)OC/C=C/c1ccc2ccnn2c1]

To a solution of 6-bromopyrazolo[1,5-a]pyridine (1.5 g, 7.61 mmol) in dioxane (50 mL) and H₂O (5 mL) was added (Z)-tert-butyldimethyl((3-(4,4,5,5-tetramethyl-1,3,2-dioxaborolan-2-yl) allyl)oxy) silane (2.50 g, 8.37 mmol), Pd(dppf)Cl₂ (0.84 g, 1.14 mmol), and K₃PO₄ (3.23 g, 15.23 mmol). The reaction was stirred for 4 h at 90° C. under nitrogen then allowed to cool to rt. The resulting mixture was diluted with water (200 mL) and extracted with EtOAc (3×300 mL). The combined organic layers were washed with brine (3×100 mL) and dried over anhydrous Na₂SO₄. After filtration, the filtrate was concentrated under reduced pressure. The residue was purified by silica gel column chromatography eluting with PE/EtOAc (5:1) to afford 1.2 g (49%) of (E)-6-(3-((tert-butyldimethylsilyl)oxy)prop-1-en-1-yl)pyrazolo[1,5-a]pyridine as a yellow oil. ¹H NMR (400 MHZ, DMSO-d₆) δ (ppm) 8.71 (s, 1H), 7.99-7.93 (m, 1H), 7.65 (d, J=9.2 Hz, 1H), 7.52-7.44 (m, 1H), 6.65-6.51 (m, 2H), 6.49-6.37 (m, 1H), 4.36-4.30 (m, 2H), 0.94-0.80 (m, 9H), 0.12-0.05 (m, 6H). MS (ESI) m/z [M+H]⁺ calcd. for C₁₆H₂₄N₂OSi, 289.2; found, 289.2.

### Step 2. Preparation of 6-(3-((tert-butyldimethylsilyl)oxy)propyl)pyrazolo[1,5-a]pyridine

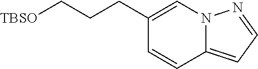 [SMILES: CC(C)(C)[Si](C)(C)OCCCc1ccc2ccnn2c1]

To a solution of (E)-6-(3-((tert-butyldimethylsilyl)oxy)prop-1-en-1-yl)pyrazolo[1,5-a]pyridine (1.2 g, 4.16 mmol, as prepared in the previous step) dissolved in MeOH (30 mL) was added 10% Pd/C (0.12 g) in a pressure tank. The mixture was hydrogenated at rt under 30 psi of hydrogen pressure for 6 h, filtered through a Celite pad and concentrated under reduced pressure. The resulting mixture was filtered, the filter cake was washed with MeOH (3×50 mL). After filtration, the filtrate was concentrated under reduced pressure to afford 1.0 g (74%) of 6-(3-((tert-butyldimethylsilyl)oxy)propyl)pyrazolo[1,5-a]pyridine as a brown oil. ¹H NMR (400 MHZ, DMSO-d₆) δ (ppm) 8.48-8.43 (m, 1H), 7.91 (d, J=2.4 Hz, 1H), 7.61 (d, J=8.8 Hz, 1H), 7.09 (dd, J=8.8, 1.2 Hz, 1H), 6.53 (dd, J=2.4, 1.2 Hz, 1H), 3.60 (t, J=6.0 Hz, 2H), 2.67-2.58 (m, 2H), 1.85-1.73 (m, 2H), 0.86 (s, 9H), 0.02 (s, 6H). MS (ESI) m/z [M+H]⁺ calcd. for C₁₆H₂₆N₂OSi, 291.2; found, 291.2.

### Step 3 Preparation of 3-bromo-6-(3-((tert-butyldimethylsilyl)oxy)propyl)pyrazolo[1,5-a]pyridine

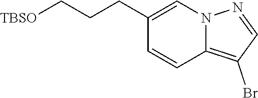 [SMILES: CC(C)(C)[Si](C)(C)OCCCc1ccc2c(Br)cnn2c1]

To a solution of 6-(3-(tert-butyldimethylsilyl)oxy)propyl)pyrazolo[1,5-a]pyridine (320 mg, 1.10 mmol, as prepared in the previous step) in DCM (10 mL) was added NBS (215.7 mg, 1.21 mmol). The reaction was stirred for 1 h at rt under nitrogen. The resulting mixture was diluted with water (20 mL) and extracted with DCM (3×20 mL). The combined organic layers were washed with brine (3×20 mL) and dried over anhydrous Na₂SO₄. After filtration, the filtrate was concentrated under reduced pressure. The residue was purified by silica gel column chromatography eluting with PE/EtOAc (7:1) to afford 210 mg (46%) of 3-bromo-6-{3-[(tert-butyldimethylsilyl)oxy]propyl}pyrazolo[1,5-a]pyridine as a yellow green oil. ¹H NMR (400 MHZ, DMSO-d₆) δ (ppm) 8.59-8.51 (m, 1H), 8.08 (s, 1H), 7.52 (d, J=9.2 Hz, 1H), 7.31-7.31 (m, 1H), 3.61 (t, J=6.0 Hz, 2H), 2.67 (t, J=7.6 Hz, 2H), 1.86-1.70 (m, 2H), 0.87-0.84 (m, 9H), 0.20 (s, 6H). MS (ESI) m/z [M+H]⁺ calcd. For C₁₆H₂₅BrN₂Osi, 369.1; found, 369.1.

### Step 4. Preparation of 3-(3-bromopyrazolo[1,5-a]pyridin-6-yl)propan-1-ol

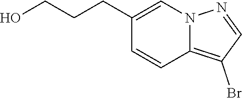 [SMILES: OCCCc1ccc2c(Br)cnn2c1]

To a solution of 3-bromo-6-(3-((tert-butyldimethylsilyl)oxy)propyl)pyrazolo[1,5-a]pyridine (600 mg, 1.62 mmol, as prepared in the previous step) dissolved in DCM (20 mL) was added HCl in MeOH (10 mL). The reaction was stirred for 1 h at 0° C. under nitrogen. The resulting mixture was diluted with water (50 mL) and extracted with DCM (3×50 mL). The combined organic layers were washed with brine (3×20 mL) and dried over anhydrous Na₂SO₄. After filtration, the filtrate was concentrated under reduced pressure. The residue was purified by Prep-TLC (PE/EtOAc 3:1) to afford 330 mg (76%) 3-(3-bromopyrazolo[1,5-a]pyridin-6-yl)propan-1-ol as a pink solid. ¹H NMR (400 MHZ, DMSO-d₆) δ (ppm) 8.56 (t, J=1.2 Hz, 1H), 8.08 (s, 1H), 7.51 (d, J=9.2 Hz, 1H), 7.27 (dd, J=9.2, 1.2 Hz, 1H), 4.54 (t, J=5.2 Hz, 1H), 3.48-3.39 (m, 2H), 2.70-2.62 (m, 2H), 1.81-1.70 (m, 2H). MS (ESI) m/z [M+H]⁺ calcd. for C₁₀H₁₁BrN₂, 255.0; found, 255.0.

### Step 5. Preparation of tert-butyl 4-(6-(3-hydroxypropyl)pyrazolo[1,5-a]pyridin-3-yl)piperazine-1-carboxylate (Compound 204)

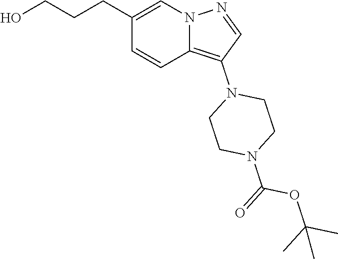 [SMILES: CC(C)(C)OC(=O)N1CCN(c2cnn3cc(CCCO)ccc23)CC1]

To a solution of 3-(3-bromopyrazolo[1,5-a]pyridin-6-yl)propan-1-ol (300 mg, 1.18 mmol, as prepared in the pervious step) in dioxane (5 mL) and tBuOH (5 mL) was added tert-butyl piperazine-1-carboxylate (1.10 g, 5.88 mmol), tBuXPhos Pd G1 (121.1 mg, 0.18 mmol), and KOtBu (197.9 mg, 1.76 mmol). The reaction was stirred for 16 h at 90° C. under nitrogen then allowed to cool to rt. The resulting mixture was diluted with water (50 mL) and extracted with EtOAc (3×50 mL). The combined organic layers were washed with brine (3×20 mL) and dried over anhydrous Na₂SO₄. After filtration, the filtrate was concentrated under reduced pressure. The crude product was purified by Prep-HPLC to afford 56.6 mg (13%) of tert-butyl 4-(6-(3-hydroxypropyl)pyrazolo[1,5-a]pyridin-3-yl)piperazine-1-carboxylate (Compound 204) as a yellow solid. ¹H NMR (400 MHZ, DMSO-d₆) δ (ppm) 8.33 (s, 1H), 7.72 (s, 1H), 7.55 (d, J=8.8 Hz, 1H), 6.99 (d, J=8.4 Hz, 1H), 4.51 (s, 1H), 3.55 (s, 4H), 3.43 (s, 2H), 2.95-2.88 (m, 4H), 2.65-2.56 (m, 2H), 1.83-1.66 (m, 2H), 1.43 (s, 9H). MS (ESI) m/z [M+H]⁺ calcd. for C₁₉H₂₈N₄O₃, 361.2; found, 361.2; HPLC purity: 254 nm: 95.5%.

### Example 148: Synthesis of Exemplary Compound 205

### tert-butyl-4-(6-(3-(pyrrolidin-1-yl)propyl)pyrazolo[1,5-a]pyridin-3-yl)piperazine-1-carboxylate (Compound 205)

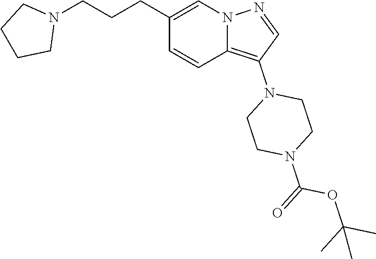 [SMILES: CC(C)(C)OC(=O)N1CCN(c2cnn3cc(CCCN4CCCC4)ccc23)CC1]

### Step 1. Preparation of (E)-1-(3-(4,4,5,5-tetramethyl-1,3,2-dioxaborolan-2-yl) allyl)pyrrolidine

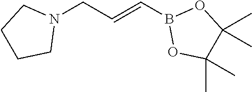 [SMILES: CC1(C)OB(/C=C/CN2CCCC2)OC1(C)C]

To a solution of 2-[(1E)-3-chloroprop-1-en-1-yl]-4,4,5,5-tetramethyl-1,3,2-dioxaborolane (3.00 g, 14.80 mmol) dissolved in ACN (30.0 mL) was added pyrrolidine (1.48 g, 20.70 mmol) and K₂CO₃ (4.09 g, 29.60 mmol). The reaction was stirred for 3 h at rt. The resulting mixture was filtered, and the filter cake was washed with ACN (3×20 mL). The filtrate was concentrated under reduced pressure to afford 2.70 g (77%) of (E)-1-(3-(4,4,5,5-tetramethyl-1,3,2-dioxaborolan-2-yl) allyl)pyrrolidine as a light yellow oil. ¹H NMR (400 MHz, DMSO-d₆) δ (ppm) 6.48 (d, J=12.0 Hz, 1H), 5.49 (d, J=8.1 Hz, 1H), 3.20-3.10 (m, 2H), 2.39-2.35 (m, 4H), 1.67-1.60 (m, 4H), 1.24-1.17 (m, 12H). MS (ESI) m/z [M+H]⁺ calcd. for C₁₃H₂₄BNO₂, 238.2; found, 238.1.

### Step 2. Preparation of (E)-6-(3-(pyrrolidin-1-yl)prop-1-en-1-yl)pyrazolo[1,5-a]pyridine

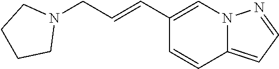 [SMILES: C(=C\c1ccc2ccnn2c1)/CN1CCCC1]

To a solution of (E)-1-(3-(4,4,5,5-tetramethyl-1,3,2-dioxaborolan-2-yl) allyl)pyrrolidine (2.70 g, 11.40 mmol, as prepared in the previous step) dissolved in dioxane (15 mL) and H₂O (1.50 mL) was added 6-bromopyrazolo[1,5-a]pyridine (2.32 g, 11.40 mmol), Pd(dppf)Cl₂·DCM (1.45 g, 1.71 mmol) and K₃PO₄ (3.76 g, 17.70 mmol). The reaction was stirred for 12 h at 90° C. under nitrogen then allowed to cool to rt. The resulting mixture was diluted with water (20 mL) and extracted with EtOAc (3×20 mL). The organic layers were combined and concentrated. The residue was purified by a silica gel column with EtOAc/PE (1/1) to afford 1.50 g (58%) of (E)-6-(3-(pyrrolidin-1-yl)prop-1-en-1-yl)pyrazolo[1,5-a]pyridine as a light yellow oil. ¹H NMR (400 MHZ, DMSO-d₆) δ (ppm) 8.70 (s, 1H), 8.02-7.94 (m, 1H), 7.72-7.63 (m, 1H), 7.50 (d, J=9.9 Hz, 1H), 6.66-6.55 (m, 2H), 6.42 (s, 1H), 3.22 (s, 2H), 2.44-7.40 (m, 4H), 1.71-1.65 (m, 4H). MS (ESI) m/z [M+H]⁺ calcd. for C₁₄H₁₇N₃, 228.1; found, 228.2.

### Step 3. Preparation of 6-(3-(pyrrolidin-1-yl)propyl)pyrazolo[1,5-a]pyridine

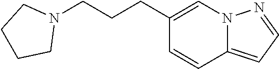 [SMILES: c1cc2ccc(CCCN3CCCC3)cn2n1]

To a solution of (E)-6-(3-(pyrrolidin-1-yl)prop-1-en-1-yl)pyrazolo[1,5-a]pyridine (1.40 g, 6.16 mmol, as prepared in the previous step) in MeOH (20.0 mL) was added Pd/C (327 mg, 3.08 mmol). The reaction was stirred for 2 h at rt under a hydrogen atmosphere. The resulting mixture was filtered, and the filter cake was washed with MeOH (3×30 mL). The filtrate was concentrated under reduced pressure to afford 600 mg (42%) of 6-(3-(pyrrolidin-1-yl)propyl)pyrazolo[1,5-a]pyridine as a light yellow oil. ¹H NMR (400 MHZ, DMSO-d₆) δ (ppm) 8.49 (s, 1H), 7.91 (d, J=2.3 Hz, 1H), 7.62 (d, J=9.0 Hz, 1H), 7.13 (dd, J=9.1, 1.5 Hz, 1H), 6.54 (d, J=2.2 Hz, 1H), 2.62 (t, J=7.6 Hz, 2H), 2.43-2.39 (m, 6H), 1.80-1.73 (m, 2H), 1.69-1.66 (m, 4H). MS (ESI) m/z [M+H]⁺ calcd. for C₁₄H₁₉N₃, 230.2; found, 230.1.

### Step 4. Preparation of 3-bromo-6-(3-(pyrrolidin-1-yl)propyl)pyrazolo[1,5-a]pyridine

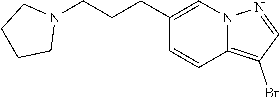 [SMILES: Brc1cnn2cc(CCCN3CCCC3)ccc12]

To a solution of 6-(3-(pyrrolidin-1-yl)propyl)pyrazolo[1,5-a]pyridine (600 mg, 2.62 mmol, as prepared in the previous step) in DCM (10 mL) was added NBS (512 mg, 2.88 mmol). The reaction was stirred for 2 h at rt. The mixture concentrated under reduced pressure then the residue was purified by a silica gel chromatography eluting with EtOAc/PE (1/1) to afford 400 mg (47%) of 3-bromo-6-(3-(pyrrolidin-1-yl)propyl)pyrazolo[1,5-a]pyridine as a light yellow oil. ¹H NMR (400 MHZ, DMSO-d₆) δ (ppm) 8.57 (s, 1H), 8.08 (s, 1H), 7.52 (d, J=9.0 Hz, 1H), 7.29 (d, J=9.2 Hz, 1H), 2.65 (t, J=7.7 Hz, 2H), 2.42-2.35 (m, 6H), 1.80-1.72 (m, 2H), 1.69-1.64 (m, 4H). MS (ESI) m/z [M+H]⁺ calcd. for C₁₄H₁₈BrN₃, 308.1; found, 308.2.

### Step 5 tert-butyl-4-(6-(3-(pyrrolidin-1-yl)propyl)pyrazolo[1,5-a]pyridin-3-yl)piperazine-1-carboxylate (Compound 205)

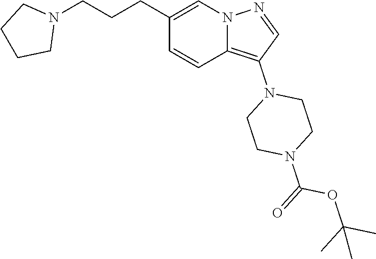 [SMILES: CC(C)(C)OC(=O)N1CCN(c2cnn3cc(CCCN4CCCC4)ccc23)CC1]

To a solution of 3-bromo-6-(3-(pyrrolidin-1-yl)propyl)pyrazolo[1,5-a]pyridine (400 mg, 1.29 mmol, as prepared in the previous step) in tBuOH (6.00 mL) and dioxane (6.00 mL) was added tert-butyl piperazine-1-carboxylate (1.21 g, 6.49 mmol), KOtBu (218 mg, 1.95 mmol) and tBuXPhos Pd G1 (133 mg, 0.20 mmol). The reaction was stirred for 16 h at 100° C. under nitrogen then allowed to cool to rt. The mixture was applied onto a silica gel column with EtOAc/PE (1/1). The crude product was purified by Prep-HPLC to afford 53.5 mg (10%) of tert-butyl-4-(6-(3-(pyrrolidin-1-yl)propyl)pyrazolo[1,5-a]pyridin-3-yl)piperazine-1-carboxylate (Compound 205) as a yellow oil. ¹H NMR (400 MHZ, DMSO-d₆) δ (ppm) 8.33 (s, 1H), 7.72 (s, 1H), 7.55 (d, J=9.1 Hz, 1H), 7.00 (d, J=9.2 Hz, 1H), 3.49-3.40 (m, 4H), 2.91 (t, J=5.1 Hz, 4H), 2.59 (d, J=7.5 Hz, 2H), 2.39-2.30 (m, 6H), 1.78-1.64 (m, 6H), 1.42 (s, 9H). MS (ESI) m/z [M+H]⁺ calcd. for C₂₃H₃₅N₅O₂, 414.3; found, 414.2; HPLC purity: 220 nm: 96.1%.

### Example 149: Synthesis of Exemplary Compound 206

### tert-butyl 4-(6-phenylpyrazolo[1,5-a]pyridin-3-yl)piperazine-1-carboxylate (Compound 206)

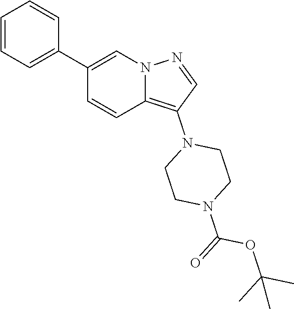 [SMILES: CC(C)(C)OC(=O)N1CCN(c2cnn3cc(-c4ccccc4)ccc23)CC1]

### Step 1. Preparation of 6-phenylpyrazolo[1,5-a]pyridine

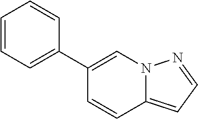 [SMILES: c1ccc(-c2ccc3ccnn3c2)cc1]

To a solution of 6-bromopyrazolo[1,5-a]pyridine (800 mg, 1.02 mmol) and phenylboronic acid (495 mg, 1.02 mmol) in dioxane (9 mL) and H₂O (1 mL) were added K₂CO₃ (1.12 g, 2.03 mmol) and Pd(dppf)Cl₂ (663.2 mg, 0.20 mmol). The reaction was stirred for 2 h at 90° C. under nitrogen then concentrated under reduced pressure. The residue was purified by Prep-TLC, eluting with PE/EtOAc (5:1) to afford 600 mg (73%) of 6-phenylpyrazolo[1,5-a]pyridine as an off-white solid. MS (ESI) m/z [M+H]⁺ calcd. for C₁₃H₁₀N₂, 195.1; found, 195.3.

### Step 2. Preparation of 3-bromo-6-phenylpyrazolo[1,5-a]pyridine

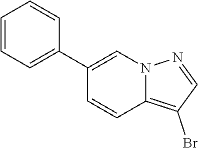 [SMILES: Brc1cnn2cc(-c3ccccc3)ccc12]

A mixture of 6-phenylpyrazolo[1,5-a]pyridine (300 mg, 1.55 mmol, as prepared in the previous step) and NBS (302.4 mg, 1.70 mmol) in DCM (5 mL) was stirred for 4 h at rt. The reaction was quenched by the addition of water (10 mL) at rt. The resulting mixture was extracted with DCM (3×20 mL). The combined organic layers were washed with brine (3×20 mL) and dried over anhydrous Na₂SO₄. After filtration, the filtrate was concentrated under reduced pressure. The residue was purified by Prep-TLC (PE/EtOAc 4:1) to afford 410 mg (94%) of 3-bromo-6-phenylpyrazolo[1,5-a]pyridine as an off-white solid. ¹H NMR (400 MHZ, DMSO-d₆) δ (ppm) 9.10 (s, 1H), 8.21 (s, 1H), 7.81-7.79 (m, 2H), 7.75-7.73 (m, 1H), 7.69-7.67 (m, 1H), 7.53-7.49 (m, 2H), 7.44-7.41 (m, 1H). MS (ESI) m/z [M+H]⁺ calcd. for C₁₃H₉BrN₂, 273.0; found, 273.2.

### Step 3. tert-butyl 4-(6-phenylpyrazolo[1,5-a]pyridin-3-yl)piperazine-1-carboxylate (Compound 206)

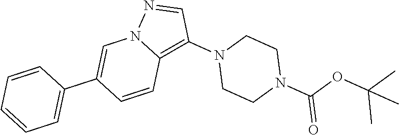 [SMILES: CC(C)(C)OC(=O)N1CCN(c2cnn3cc(-c4ccccc4)ccc23)CC1]

To a stirred mixture of 3-bromo-6-phenylpyrazolo[1,5-a]pyridine (400 mg, 1.46 mmol, as prepared in the previous step) and tert-butyl piperazine-1-carboxylate (1.36 g, 7.32 mmol) in dioxane (4 mL) and tBuOH (8 mL) were added KOtBu (328.7 mg, 2.93 mmol) and tBuXPhos Pd G1 (201.1 mg, 0.29 mmol) at rt. The reaction was stirred overnight at 80° C. under nitrogen then allowed to cool to rt. The reaction was quenched by the addition of water (20 mL) at rt. The resulting mixture was extracted with DCM (3×40 mL). The combined organic layers were washed with brine (3×40 mL) and dried over anhydrous Na₂SO₄. After filtration, the filtrate was concentrated under reduced pressure. The residue was purified by silica gel column chromatography eluting with PE/EtOAc (1:1) to afford the crude product. The crude product was purified by Prep-HPLC to afford 73.9 mg (13%) of tert-butyl 4-(6-phenylpyrazolo[1,5-a]pyridin-3-yl)piperazine-1-carboxylate (Compound 206) as a yellow solid. ¹H NMR (400 MHZ, DMSO-d₆) δ (ppm) 8.87 (s, 1H), 7.86 (s, 1H), 7.77-7.72 (m, 3H), 7.51-7.37 (m, 4H), 3.53 (t, J=4.4 Hz, 4H), 2.97 (t, J=5.2 Hz, 4H), 1.44 (s, 9H). MS (ESI) m/z [M+H]⁺ calcd. for C₂₂H₂₆N₄O₂, 379.2; found, 379.15; HPLC purity: 254 nm: 99.1%.

### Example 150: Synthesis of Exemplary Compound 207

### tert-butyl 4-(6-(4-fluorophenyl)pyrazolo[1,5-a]pyridin-3-yl)piperazine-1-carboxylate (Compound 207)

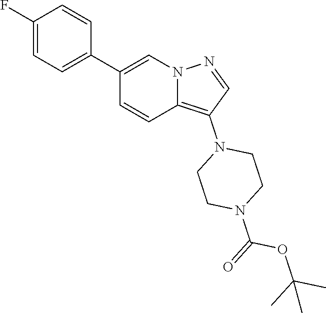 [SMILES: CC(C)(C)OC(=O)N1CCN(c2cnn3cc(-c4ccc(F)cc4)ccc23)CC1]

### Step 1. Preparation of 6-(4-fluorophenyl)pyrazolo[1,5-a]pyridine

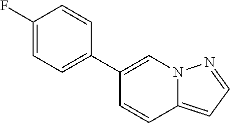 [SMILES: Fc1ccc(-c2ccc3ccnn3c2)cc1]

To a solution of 6-bromopyrazolo[1,5-a]pyridine (1 g, 5.07 mmol) and 4-fluorophenylboronic acid (852.1 mg, 6.01 mmol) in dioxane (20 mL) and H₂O (2 mL) were added K₃PO₄ (2.15 g, 10.15 mmol) and Pd-PEPPSI-IPent^{Cl} 2-methylpyridine (853.8 mg, 1.01 mmol). The reaction was stirred for 16 h at 90° C. under nitrogen. The mixture was allowed to cool to rt then concentrated under reduced pressure. The residue was diluted with water and extracted with EtOAc (3×50 mL). The combined organic layers were washed with brine (10 mL) and dried over anhydrous Na₂SO₄. After filtration, the filtrate was concentrated under reduced pressure. The residue was purified by silica gel column chromatography eluting with PE/EtOAc (1:1) to afford 710 mg (65%) of 6-(4-fluorophenyl)pyrazolo[1,5-a]pyridine as a yellow solid. ¹H NMR (400 MHZ, DMSO-d₆) δ (ppm) 9.04 (s, 1H), 8.05 (s, 1H), 7.87-7.76 (m, 3H), 7.56 (d, J=9.3 Hz, 1H), 7.33 (t, J=8.7 Hz, 2H), 6.65 (s, 1H); MS (ESI) m/z [M+H]⁺ calcd. for C₁₃H₉FN₂, 213.2; found, 213.1.

### Step 2. Preparation of 3-bromo-6-(4-fluorophenyl)pyrazolo[1,5-a]pyridine

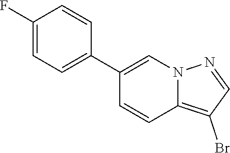 [SMILES: Fc1ccc(-c2ccc3c(Br)cnn3c2)cc1]

To a solution of 6-(4-fluorophenyl)pyrazolo[1,5-a]pyridine (700 mg, 3.30 mmol, as prepared in the previous step) in DCM (10 mL) was added NBS (704.5 mg, 3.96 mmol). The reaction was stirred for 2 h at rt under a nitrogen then concentrated under reduced pressure. The residue was diluted with water and extracted with EtOAc (3×50 mL). The combined organic layers were washed with brine (10 mL) and dried over anhydrous Na₂SO₄. After filtration, the filtrate was concentrated under reduced pressure. The residue was purified by Prep-TLC MeOH/H₂O (10:1) to afford 850 mg (89%) of 3-bromo-6-(4-fluorophenyl)pyrazolo[1,5-a]pyridine as a yellow solid. ¹H NMR (400 MHZ, DMSO-d₆) δ (ppm) 8.21-8.10 (m, 1H), 7.38-7.30 (m, 1H), 7.07-6.85 (m, 4H), 6.59-6.46 (m, 2H). MS (ESI) m/z [M+H]⁺ calcd. for C₁₃H₈BrFN₂, 291.1 found, 291.0.

### Step 3. tert-butyl 4-(6-(4-fluorophenyl)pyrazolo[1,5-a]pyridin-3-yl)piperazine-1-carboxylate (Compound 207)

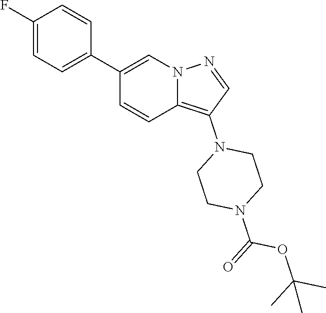 [SMILES: CC(C)(C)OC(=O)N1CCN(c2cnn3cc(-c4ccc(F)cc4)ccc23)CC1]

To a solution of 3-bromo-6-(4-fluorophenyl)pyrazolo[1,5-a]pyridine (500 mg, 1.72 mmol, as prepared in the previous step) and tert-butyl piperazine-1-carboxylate (1.60 g, 8.58 mmol) in tBuOH (10 mL) and dioxane (10 mL) were added KOtBu (385.5 mg, 3.43 mmol) and tBuXPhos Pd G3 (272.8 mg, 0.34 mmol). The reaction was stirred for 16 h at 90° C. under nitrogen then concentrated under reduced pressure. The resulting mixture was diluted with water (20 mL) and extracted with EtOAc (3×200 mL). The combined organic layers were washed with brine (10 mL) and dried over anhydrous Na₂SO₄. After filtration, the filtrate was concentrated under reduced pressure. The residue was purified by silica gel column chromatography eluting with PE/EtOAc (9:1) to afford crude product. The crude product was purified by Prep-HPLC to afford 66.2 mg (10%) of tert-butyl 4-(6-(4-fluorophenyl)pyrazolo[1,5-a]pyridin-3-yl)piperazine-1-carboxylate (Compound 207) as a white solid. ¹H NMR (400 MHZ, DMSO-d₆) δ (ppm) 8.88 (s, 1H), 7.86 (s, 1H), 7.85-7.77 (m, 2H), 7.73 (d, J=9.3 Hz, 1H), 7.46-7.39 (m, 1H), 7.32 (t, J=8.8 Hz, 2H), 3.53 (t, J=5.0 Hz, 4H), 2.97 (t, J=5.0 Hz, 4H), 1.44 (s, 9H). MS (ESI) m/z [M+H]⁺ calcd. for C₂₂H₂₅FN₄O₂, 397.2; found, 397.1; HPLC purity: 254 nm: 99.8%.

### Example 151: Synthesis of Exemplary Compound 208

### tert-butyl 4-(6-(3-fluorophenyl)pyrazolo[1,5-a]pyridin-3-yl)piperazine-1-carboxylate (Compound 208)

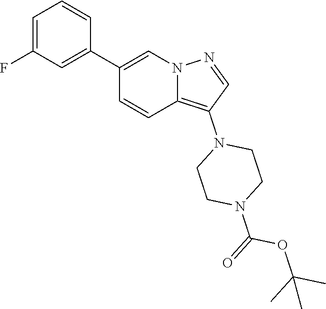 [SMILES: CC(C)(C)OC(=O)N1CCN(c2cnn3cc(-c4cccc(F)c4)ccc23)CC1]

### Step 1. Preparation of 6-(3-fluorophenyl)pyrazolo[1,5-a]pyridine

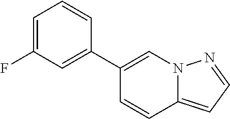 [SMILES: Fc1cccc(-c2ccc3ccnn3c2)c1]

To a solution of 6-bromopyrazolo[1,5-a]pyridine (500 mg, 2.54 mmol) in dioxane (10 mL) and H₂O (1 mL) was added Pd(dppf)Cl₂ (278 mg, 0.38 mmol), K₃PO₄ (1.6 g, 7.61 mmol) and (3-fluorophenyl) boronic acid (532 mg, 3.81 mmol) at rt. The reaction was stirred for 6 h at 90° C. The resulting mixture was diluted with water (200 mL) and extracted with EtOAc (3×200 mL). The combined organic layers were dried over anhydrous Na₂SO₄. After filtration, the filtrate was concentrated under reduced pressure. The residue was purified by silica gel column chromatography eluting with PE/EtOAc (9:1) to afford 500 mg (93%) 6-(3-fluorophenyl)pyrazolo[1,5-a]pyridine as a white solid. ¹H NMR (400 MHZ, DMSO-d₆) δ (ppm) 9.12 (d, J=1.6 Hz, 1H), 8.06 (d, J=2.3 Hz, 1H), 7.79 (d, J=9.2 Hz, 1H), 7.72-7.46 (m, 4H), 7.21-7.18 (m, 1H), 6.66 (d, J=2.2 Hz, 1H). MS (ESI) m/z [M+H]⁺ calcd. for C₁₃H₉FN₂, 213.1; found, 213.1.

### Step 2. Preparation of 3-bromo-6-(3-fluorophenyl)pyrazolo[1,5-a]pyridine

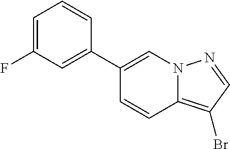 [SMILES: Fc1cccc(-c2ccc3c(Br)cnn3c2)c1]

To a solution of 6-(3-fluorophenyl)pyrazolo[1,5-a]pyridine (500 mg, 2.36 mmol, as prepared in the previous step) in DCM (4 mL) was added NBS (461 mg, 2.59 mmol). The reaction was stirred for 2 h at rt. The mixture was purified by silica gel column chromatography eluting with PE/EtOAc (9:1) to afford 500 mg (72.9%) of 3-bromo-6-(3-fluorophenyl)pyrazolo[1,5-a]pyridine as a white solid. ¹H NMR (400 MHZ, DMSO-d₆) δ (ppm) 9.17 (s, 1H), 8.21 (s, 1H), 7.74-7.72 (m, 2H), 7.70-7.64 (m, 2H), 7.63 (t, J=1.1 Hz, 1H), 7.23-7.21 (m, 1H). MS (ESI) m/z [M+H]⁺ calcd. for C₁₃H₈BrFN₂, 291.0; found, 291.0.

### Step 3. tert-butyl 4-(6-(3-fluorophenyl)pyrazolo[1,5-a]pyridin-3-yl)piperazine-1-carboxylate (Compound 208)

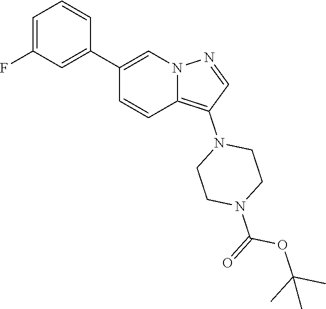 [SMILES: CC(C)(C)OC(=O)N1CCN(c2cnn3cc(-c4cccc(F)c4)ccc23)CC1]

To a solution of 3-bromo-6-(3-fluorophenyl)pyrazolo[1,5-a]pyridine (500 mg, 1.72 mmol, as prepared in the previous step) in tBuOH (10 mL) and dioxane (5 mL) was added tBuXPhos Pd G3 (0.2 g, 0.26 mmol), KOtBu (290 mg, 2.58 mmol) and tert-butyl piperazine-1-carboxylate (1.5 g, 8.59 mmol). The reaction was stirred for 18 h at 60° C. The resulting mixture was diluted with water (200 mL) and extracted with EtOAc (3×200 mL). The combined organic layers were dried over anhydrous Na₂SO₄. After filtration, the filtrate was concentrated under reduced pressure. The residue was purified by silica gel column chromatography eluting with PE/EtOAc (9:1) to afford crude product. The crude product was purified by Prep-HPLC to afford 118.6 mg (17%) of tert-butyl 4-[6-(3-fluorophenyl)pyrazolo[1,5-a]pyridin-3-yl]piperazine-1-carboxylate (Compound 208) as a yellow solid. ¹H NMR (400 MHZ, DMSO-d₆) δ (ppm) 8.98 (s, 1H), 7.89 (s, 1H), 7.74-7.72 (m, 1H), 7.70-7.60 (m, 2H), 7.57-7.47 (m, 2H), 7.22-7.20 (m, 1H), 3.53 (t, J=5.0 Hz, 4H), 2.97 (t, J=5.0 Hz, 4H), 1.44 (s, 9H). MS (ESI) m/z [M+H]⁺ calcd. for C₂₂H₂₅FN₄O₂, 397.2; found, 397.1; HPLC purity: 254 nm: 97.5%.

### Example 152: Synthesis of Exemplary Compound 209

### tert-butyl-4-(6-(2-fluorophenyl)pyrazolo[1,5-a]pyridin-3-yl)piperazine-1-carboxylate (Compound 209)

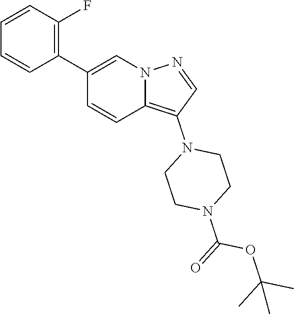 [SMILES: CC(C)(C)OC(=O)N1CCN(c2cnn3cc(-c4ccccc4F)ccc23)CC1]

### Step 1. Preparation of 6-(2-fluorophenyl)pyrazolo[1,5-a]pyridine

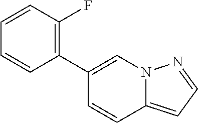 [SMILES: Fc1ccccc1-c1ccc2ccnn2c1]

To a solution of 6-bromopyrazolo[1,5-a]pyridine (2.00 g, 10.20 mmol) dissolved in dioxane (15.0 mL) and H₂O (1.50 mL) was added 2-fluorophenylboronic acid (2.14 g, 15.30 mmol), Pd(dppf)Cl₂·DCM (1.24 g, 1.53 mmol) and K₃PO₄ (6.46 g, 30.6 mmol). The reaction was stirred 90° C. for 12 h under nitrogen then allowed to cool to rt. The resulting mixture was diluted with water (20 mL) and extracted with EtOAc (3×30 mL). The organic layers were combined and concentrated. The residue was purified by a silica gel column with EtOAc/PE (1/1) to afford 2.00 g (92%) of 6-(2-fluorophenyl)pyrazolo[1,5-a]pyridine as a white solid: 1H NMR (400 MHZ, DMSO-d₆) δ (ppm) 8.89 (s, 1H), 8.07 (d, J=2.3 Hz, 1H), 7.80 (dd, J=9.2, 1.0 Hz, 1H), 7.70-7.65 (m, 1H), 7.50-7.31 (m, 4H), 6.68 (d, J=4.0 Hz, 1H). MS (ESI) m/z [M+H]⁺ calcd. for C₁₃H₉FN₂, 213.1; found, 213.2.

### Step 2. Preparation of 3-bromo-6-(2-fluorophenyl)pyrazolo[1,5-a]pyridine

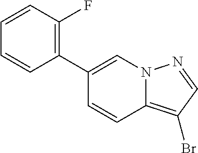 [SMILES: Fc1ccccc1-c1ccc2c(Br)cnn2c1]

To a solution of 6-(2-fluorophenyl)pyrazolo[1,5-a]pyridine (1.00 g, 4.71 mmol, as prepared in the previous step) dissolved in DCM (20.0 mL) was added NBS (0.92 g, 5.18 mmol). The reaction was stirred at rt for 2 h then concentrated under reduced pressure. The residue was purified by a silica gel column with EtOAc/PE (1/1) to afford 1.10 g (79%) of 3-bromo-6-(2-fluorophenyl)pyrazolo[1,5-a]pyridine as a yellow solid. ¹H NMR (400 MHZ, DMSO-d₆) δ (ppm) 8.97 (s, 1H), 8.24 (s, 1H), 7.72-7.67 (m, 2H), 7.60-7.56 (m, 1H), 7.53-7.46 (m, 1H), 7.42-7.33 (m, 2H). MS (ESI) m/z [M+H]⁺ calcd. for C₁₃H₈BᵣFN₂, 291.0; found, 291.1.

### Step 3. tert-butyl-4-(6-(2-fluorophenyl)pyrazolo[1,5-a]pyridin-3-yl)piperazine-1-carboxylate (Compound 209)

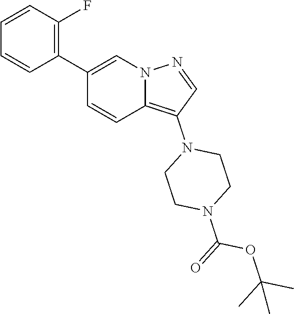 [SMILES: CC(C)(C)OC(=O)N1CCN(c2cnn3cc(-c4ccccc4F)ccc23)CC1]

To a solution of 3-bromo-6-(2-fluorophenyl)pyrazolo[1,5-a]pyridine (500 mg, 1.72 mmol, as prepared in the previous step) dissolved in tBuOH (5 mL) and dioxane (5.00 mL) was added tert-butyl piperazine-1-carboxylate (1.60 g, 8.58 mmol), KOtBu (289 mg, 2.57 mmol) and tBuXPhos Pd G1 (177 mg, 0.26 mmol). The reaction was stirred 90° C. for 16 h under nitrogen then allowed to cool to rt. The resulting mixture was diluted with water (15.0 mL) and extracted with EtOAc (3×30 mL). The organic layers were combined and concentrated under reduced pressure. The residue was applied onto a silica gel column eluting with EtOAc/PE (1/1). The crude product was purified by Prep-HPLC to afford 145.6 mg (20%) of tert-butyl-4-(6-(2-fluorophenyl)pyrazolo[1,5-a]pyridin-3-yl)piperazine-1-carboxylate (Compound 209) as a yellow solid. ¹H NMR (400 MHZ, DMSO-d₆) δ (ppm) 8.74 (s, 1H), 7.90 (s, 1H), 7.74 (d, J=9.3 Hz, 1H), 7.69-7.64 (m, 1H), 7.49-7.43 (m, 1H), 7.40-7.26 (m, 3H), 3.53 (t, J=4.9 Hz, 4H), 2.98 (t, J=5.0 Hz, 4H), 1.44 (s, 9H). MS (ESI) m/z [M+H]⁺ calcd. for C₂₂H₂₅FN₄O₂, 397.2; found, 397.1; HPLC purity: 254 nm: 95.4%.

### Example 153: Synthesis of Exemplary Compound 210

### tert-butyl 4-(6-(4-methoxyphenyl)pyrazolo[1,5-a]pyridin-3-yl)piperazine-1-carboxylate (Compound 210)

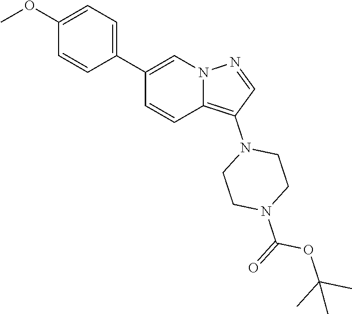 [SMILES: COc1ccc(-c2ccc3c(N4CCN(C(=O)OC(C)(C)C)CC4)cnn3c2)cc1]

### Step 1. Preparation of 6-(4-methoxyphenyl)pyrazolo[1,5-a]pyridine

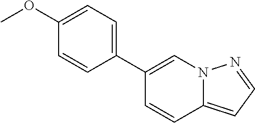 [SMILES: COc1ccc(-c2ccc3ccnn3c2)cc1]

A solution of 6-bromopyrazolo[1,5-a]pyridine (500 mg, 2.53 mmol) and 4-methoxyphenylboronic acid (462.7 mg, 3.05 mmol), K₂CO₃ (701.4 mg, 5.07 mmol), Pd(dppf)Cl₂·DCM (413.4 mg, 0.50 mmol) in dioxane (10 mL) and H₂O (1 mL) was stirred for 3 h at 90° C. under then allowed to cool to rt. The resulting mixture was concentrated under reduced pressure. The residue was dissolved in EtOAc (10 mL). The resulting mixture was filtered, then the filter cake was washed with EtOAc (2×10 mL). The filtrate was concentrated under reduced pressure. The residue was purified by silica gel column chromatography eluting with PE/EtOAc (1:1) to afford 650 mg (114%) 6-(4-methoxyphenyl)pyrazolo[1,5-a]pyridine as a yellow solid. ¹H NMR (400 MHZ, DMSO-d₆) δ (ppm) 8.96 (s, 1H), 8.02 (t, J=2.5 Hz, 1H), 7.80-7.68 (m, 3H), 7.55 (d, J=8.9 Hz, 1H), 7.09-7.00 (m, 2H), 6.63 (d, J=3.2 Hz, 1H), 3.75 (s, 3H). MS (ESI) m/z [M+H]⁺ calcd. for C₁₄H₁₂N₂O, 224.1; found, 224.1.

### Step 2. 3-bromo-6-(4-methoxyphenyl)pyrazolo[1,5-a]pyridine

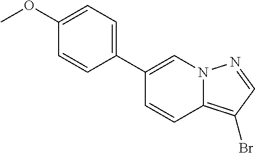 [SMILES: COc1ccc(-c2ccc3c(Br)cnn3c2)cc1]

A solution of 6-(4-methoxyphenyl)pyrazolo[1,5-a]pyridine (630 mg, 2.80 mmol, as prepared in the previous step) and NBS (550 mg, 3.09 mmol) in DCM (10 mL) was stirred for 3 h at rt. The resulting mixture was concentrated under reduced pressure. The residue was purified by silica gel column chromatography eluting with PE/EtOAc (1:1) to afford 600 mg (71%) of 3-bromo-6-(4-methoxyphenyl)pyrazolo[1,5-a]pyridine as a yellow solid. ¹H NMR (400 MHZ, DMSO-d₆) δ (ppm) 9.02 (s, 1H), 8.18 (s, 1H), 7.78-7.60 (m, 4H), 7.10-7.01 (m, 2H), 3.82 (s, 3H). MS (ESI) m/z [M+H]⁺ calcd. for C₁₄H₁₁BrN₂O, 302.0; found, 302.0.

### Step 3. tert-butyl 4-(6-(4-methoxyphenyl)pyrazolo[1,5-a]pyridin-3-yl)piperazine-1-carboxylate (Compound 210)

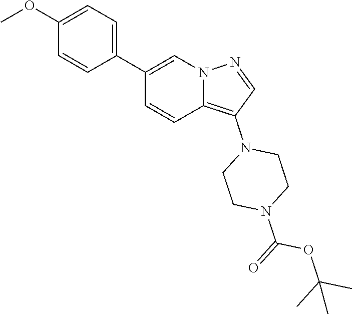 [SMILES: COc1ccc(-c2ccc3c(N4CCN(C(=O)OC(C)(C)C)CC4)cnn3c2)cc1]

A solution of 6-(4-methoxyphenyl)pyrazolo[1,5-a]pyridine (300 mg, 1.33 mmol, as prepared in the previous step) and tert-butyl piperazine-1-carboxylate (1.25 g, 6.69 mmol), KOtBu (300.2 mg, 2.67 mmol), tBuXPhos Pd G1 (110.2 mg, 0.16 mmol) in tBuOH (20 mL) and dioxane (2 mL) was stirred for 2 h at 90° C. under nitrogen then allowed to cool to rt. The resulting mixture was concentrated under reduced pressure. Then residue was dissolved in EtOAc (10 mL). The resulting mixture was filtered, and the filter cake was washed with EtOAc (2×10 mL). The filtrate was concentrated under reduced pressure. The residue was purified by Prep-TLC (PE/EtOAc 1:1) to afford crude product. The crude product was purified by Prep-HPLC to afford 66.5 mg (12%) of tert-butyl 4-[6-(4-methoxyphenyl)pyrazolo[1,5-a]pyridin-3-yl]piperazine-1-carboxylate (Compound 210) as a yellow solid. ¹H NMR (400 MHz, DMSO-d₆) δ (ppm) 8.79 (s, 1H), 7.83 (s, 1H), 7.70 (d, J=9.0 Hz, 3H), 7.41 (dd, J=9.3, 1.6 Hz, 1H), 7.08-6.98 (m, 2H), 3.81 (s, 3H), 3.52 (t, J=5.0 Hz, 4H), 2.96 (t, J=5.0 Hz, 4H), 1.44 (s, 9H). MS (ESI) m/z [M+H]⁺ calcd. for C₂₃H₂₈N₄O₃, 409.1; found, 409.1; HPLC purity: 254 nm: 97.6%.

### Example 154: Synthesis of Exemplary Compound 211

### tert-butyl 4-(6-(3-methoxyphenyl)pyrazolo[1,5-a]pyridin-3-yl)piperazine-1-carboxylate (Compound 211)

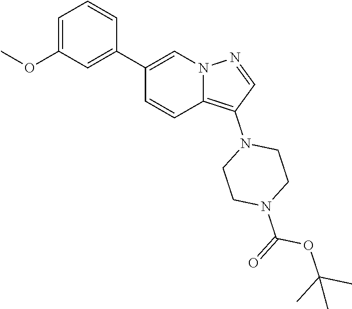 [SMILES: COc1cccc(-c2ccc3c(N4CCN(C(=O)OC(C)(C)C)CC4)cnn3c2)c1]

### Step 1. Preparation of 6-(3-methoxyphenyl)pyrazolo[1,5-a]pyridine

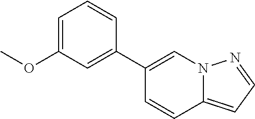 [SMILES: COc1cccc(-c2ccc3ccnn3c2)c1]

To a stirred solution of 6-bromopyrazolo[1,5-a]pyridine (1 g, 5.07 mmol) and (3-methoxyphenyl) boronic acid (771 mg, 5.07 mmol) in dioxane (16 mL) and H₂O (1.6 mL) were added XPhos (242 mg, 0.51 mmol), XPhos Pd G3 (430 mg, 0.51 mmol) and K₃PO₄ (2.15 g, 10.15 mmol) at rt. The reaction was stirred for 1 h at 80° C. under nitrogen. The resulting mixture was filtered, the filter cake was washed with DCM (3×70 mL). The filtrate was concentrated under reduced pressure. The residue was purified by silica gel column chromatography eluting with PE/EtOAc (5:1) to afford 1.1 g (97%) of 6-(3-methoxyphenyl)pyrazolo[1,5-a]pyridine as a yellow solid. ¹H NMR (400 MHZ, DMSO-d₆) δ (ppm) 9.06 (s, 1H), 8.05 (d, J=2.2 Hz, 1H), 7.79 (d, J=9.2 Hz, 1H), 7.59 (dd, J=9.2, 1.6 Hz, 1H), 7.40 (t, J=7.9 Hz, 1H), 7.38-7.30 (m, 2H), 7.00-6.94 (m, 1H), 6.65 (d, J=1.7 Hz, 1H), 3.85 (s, 3H). MS (ESI) m/z [M+H]⁺ calcd. for C₁₄H₁₂N₂O, 225.1; found, 225.2.

### Step 2. Preparation of 3-bromo-6-(3-methoxyphenyl)pyrazolo[1,5-a]pyridine

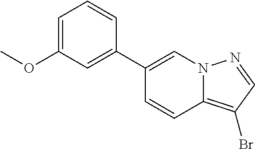 [SMILES: COc1cccc(-c2ccc3c(Br)cnn3c2)c1]

To a stirred solution of 6-(3-methoxyphenyl)pyrazolo[1,5-a]pyridine (1 g, 4.46 mmol, as prepared in the previous step) in DCM (15 mL) was added NBS (0.87 g, 4.91 mmol) at rt. The reaction was stirred for 1 h at rt. The resulting mixture was concentrated under reduced pressure. The residue was purified by reverse flash chromatography with C₁₈ silica gel column eluting with 10% to 100% ACN in water (10 mmol/L NH₄HCO₃) to afford 780 mg (58%) of 3-bromo-6-(3-methoxyphenyl)pyrazolo[1,5-a]pyridine as a white solid. ¹H NMR (400 MHz, DMSO-d₆) δ (ppm) 9.13 (s, 1H), 8.21 (s, 1H), 7.74 (dd, J=9.3, 1.6 Hz, 1H), 7.66 (d, J=9.2 Hz, 1H), 7.41 (t, J=8.1 Hz, 1H), 7.37-7.31 (m, 2H), 7.02-6.96 (m, 1H), 3.86 (s, 3H). MS (ESI) m/z [M+H]⁺ calcd. for C₁₄H₁₁BrN₂O, 303.0; found, 303.1.

### Step 3. Preparation of tert-butyl 4-(6-(3-methoxyphenyl)pyrazolo[1,5-a]pyridin-3-yl)piperazine-1-carboxylate (Compound 211)

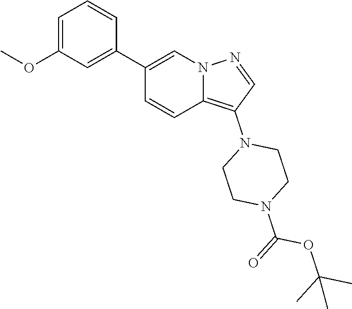 [SMILES: COc1cccc(-c2ccc3c(N4CCN(C(=O)OC(C)(C)C)CC4)cnn3c2)c1]

To a stirred solution of 3-bromo-6-(3-methoxyphenyl)pyrazolo[1,5-a]pyridine (600 mg, 1.98 mmol, as prepared in the previous step) and tert-butyl piperazine-1-carboxylate (1.84 g, 9.90 mmol) in tBuOH (5 mL) and dioxane (5 mL) were added KOtBu (333 mg, 2.97 mmol) and tBuXPhos Pd G1 (204 mg, 0.30 mmol) at rt. The reaction was stirred overnight at 60° C. under nitrogen. The resulting mixture was filtered, the filter cake was washed with DCM (3×80 mL). The filtrate was concentrated under reduced pressure. The residue was purified by silica gel column chromatography eluting with DCM/MeOH (15:1) and then purified by Prep-HPLC to afford 155.1 mg (19%) of tert-butyl 4-(6-(3-methoxyphenyl)pyrazolo[1,5-a]pyridin-3-yl)piperazine-1-carboxylate (Compound 211) as a yellow solid. ¹H NMR (400 MHz, DMSO-d₆) δ (ppm) 8.91 (s, 1H), 7.87 (s, 1H), 7.72 (d, J=9.3 Hz, 1H), 7.48-7.36 (m, 2H), 7.35-7.28 (m, 2H), 6.98-6.92 (m, 1H), 3.85 (s, 3H), 3.53 (t, J=4.9 Hz, 4H), 2.97 (t, J=5.0 Hz, 4H), 1.44 (s, 9H). MS (ESI) m/z [M+H]⁺ calcd. for C₂₃H₂₈N₄O₃, 409.2; found, 409.25; HPLC purity: 254 nm: 99.7%.

### Example 155: Synthesis of Exemplary Compound 212

### tert-butyl 4-(6-(2-methoxyphenyl)pyrazolo[1,5-a]pyridin-3-yl)piperazine-1-carboxylate (Compound 212)

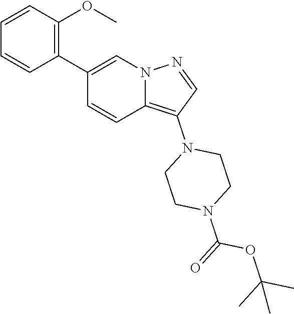 [SMILES: COc1ccccc1-c1ccc2c(N3CCN(C(=O)OC(C)(C)C)CC3)cnn2c1]

### Step 1. Preparation of 6-(2-methoxyphenyl)pyrazolo[1,5-a]pyridine

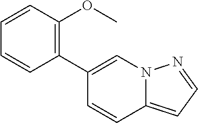 [SMILES: COc1ccccc1-c1ccc2ccnn2c1]

To a solution of 6-bromopyrazolo[1,5-a]pyridine (800 mg, 1.02 mmol) and 2-methoxyphenylboronic acid (617.0 mg, 1.06 mmol) in dioxane (9 mL) and H₂O (1 mL) were added K₂CO₃ (1.12 g, 2.03 mmol) and Pd(dppf)Cl₂ (663.2 mg, 0.20 mmol). The reaction was stirred overnight at 90° C. under nitrogen then concentrated under reduced pressure. The residue was purified by silica gel column chromatography eluting with PE/EtOAc (5:1) to afford 720 mg (79%) of 6-(2-methoxyphenyl)pyrazolo[1,5-a]pyridine as an off-white solid. MS (ESI) m/z [M+H]⁺ calcd. for C₁₄H₁₂N₂O, 225.1; found, 225.2.

### Step 2. Preparation of 3-bromo-6-(2-methoxyphenyl)pyrazolo[1,5-a]pyridine

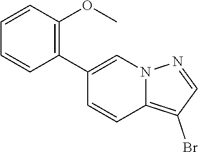 [SMILES: COc1ccccc1-c1ccc2c(Br)cnn2c1]

A mixture of 6-(2-methoxyphenyl)pyrazolo[1,5-a]pyridine (300 mg, 1.55 mmol, as prepared in the previous step) and NBS (302.4 mg, 1.70 mmol) in DCM (5 mL) was stirred for 4 h at rt. The reaction was quenched by the addition of water (10 mL) at rt. The resulting mixture was extracted with DCM (3×20 mL). The combined organic layers were washed with brine (3×20 mL) and dried over anhydrous Na₂SO₄. After filtration, the filtrate was concentrated under reduced pressure. The residue was purified by Prep-TLC PE/EtOAc (4:1) to afford 420 mg (94%) of 3-bromo-6-phenylpyrazolo[1,5-a]pyridine as an off-white solid. ¹H NMR (400 MHZ, DMSO-d₆) δ (ppm) 8.87 (s, 1H), 8.81 (s, 1H), 7.64-7.60 (m, 1H), 7.53-7.51 (m, 1H), 7.50-7.40 (m, 2H), 7.17 (d, J=8. 0 Hz, 1H), 7.10-7.06 (m, 1H), 3.82 (s, 3H). MS (ESI) m/z [M+H]⁺ calcd. for C₁₄H₁₁BrN₂O, 303.0; found, 303.2.

### Step 3. tert-butyl 4-(6-(2-methoxyphenyl)pyrazolo[1,5-a]pyridin-3-yl)piperazine-1-carboxylate (Compound 212)

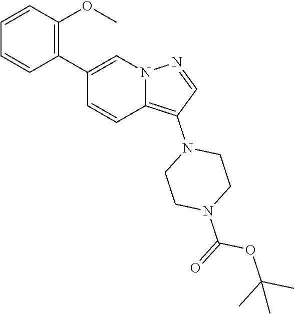 [SMILES: COc1ccccc1-c1ccc2c(N3CCN(C(=O)OC(C)(C)C)CC3)cnn2c1]

To a stirred mixture of 3-bromo-6-(2-methoxyphenyl)pyrazolo[1,5-a]pyridine (400 mg, 1.32 mmol, as prepared in the previous step) and tert-butyl piperazine-1-carboxylate (1.23 g, 6.59 mmol) in dioxane (4 mL) and tBuOH (8 mL) were added KOtBu (296.1 mg, 2.64 mmol) and tBuXPhos Pd G1 (181.2 mg, 0.26 mmol) at rt. The reaction was stirred overnight at 80° C. under nitrogen then allowed to cool to rt. The reaction was quenched by the addition of water (20 mL) at rt. The resulting mixture was extracted with DCM (3×40 mL). The combined organic layers were washed with brine (3×40 mL) and dried over anhydrous Na₂SO₄. After filtration, the filtrate was concentrated under reduced pressure. The residue was purified by silica gel column chromatography eluting with PE/EtOAc (1:1) to afford the crude product. The crude product was purified by Prep-HPLC to afford 50.3 mg (9%) of tert-butyl 4-(6-(2-methoxyphenyl)pyrazolo[1,5-a]pyridin-3-yl)piperazine-1-carboxylate (Compound 212) as a yellow solid. ¹H NMR (400 MHZ, DMSO-d₆) δ (ppm) 8.59 (s, 1H), 7.84 (s, 1H), 7.65 (d, J=9.2 Hz, 1H), 7.44-7.37 (m, 2H), 7.23 (d, J=9.2 Hz, 1H), 7.16 (d, J=8.0 Hz, 1H), 7.06 (t, J=7.6 Hz, 1H), 3.82 (s, 3H), 3.52 (s, 4H), 2.96 (t, J=4.8 Hz, 4H), 1.44 (s, 9H). MS (ESI) m/z [M+H]⁺ calcd. for C₂₃H₂₈N₄O₃, 409.2; found, 409.25; HPLC purity: 254 nm: 97.3%.

### Example 156: Synthesis of Exemplary Compound 213

### tert-butyl 4-{6-cyclopropylpyrazolo[1,5-a]pyridin-3-yl}piperazine-1-carboxylate (Compound 213)

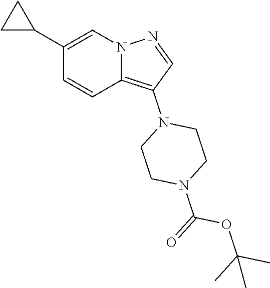 [SMILES: CC(C)(C)OC(=O)N1CCN(c2cnn3cc(C4CC4)ccc23)CC1]

### Step. 1 Synthesis of 6-cyclopropylpyrazolo[1,5-a]pyridine

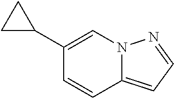 [SMILES: c1cc2ccc(C3CC3)cn2n1]

To a stirred solution of 6-bromopyrazolo[1,5-a]pyridine (2 g, 10.15 mmol) in dioxane (20 mL) and H₂O (2 mL) was added cyclopropylboronic acid (1.05 g, 12.18 mmol), Pd(dppf)Cl₂ (0.15 g, 0.21 mmol) and Cs₂CO₃ (6.61 g, 20.30 mmol). The reaction was stirred for 5 h at 90° C. under nitrogen then concentrated under vacuum. Water (50 mL) was added to the resulting residue, which was extracted with EtOAc (2×100 mL). The combined organic layers were washed with brine (2×5 mL) and dried over anhydrous Na₂SO₄. After filtration, the filtrate was concentrated under reduced pressure. The residue was purified by Prep-TLC (DCM/MeOH 12:1) to afford 6-cyclopropylpyrazolo[1,5-a]pyridine as a light yellow oil. ¹H NMR (400 MHZ, DMSO-d₆) δ 8.53-8.45 (m, 1H), 7.90 (d, J=2.3 Hz, 1H), 7.59 (m, 1H), 6.94 (m, 1H), 6.53 (m, 1H), 1.98 (m, 1H), 0.98-0.87 (m, 2H), 0.81-0.70 (m, 2H). MS (ESI) m/z [M+H]⁺ calcd. for C₁₀H₁₀N₂, 159.1; found, 159.2.

### Step 2. Synthesis of 3-bromo-6-cyclopropylpyrazolo[1,5-a]pyridine

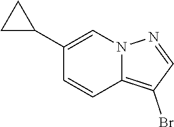 [SMILES: Brc1cnn2cc(C3CC3)ccc12]

To a stirred solution of 6-cyclopropylpyrazolo[1,5-a]pyridine (650 mg, 4.11 mmol, as prepared in the previous step) in DCM (20 mL) was added NBS (877.5 mg, 4.93 mmol) at rt. The resulting mixture was stirred for 2 h at rt. The reaction was quenched with sat. aq. sodium thiosulfate at 0° C. To the resulting mixture was added water (50 mL), which was extracted with DCM (2×100 mL). The combined organic layers were washed with brine (2×5 mL) and dried over anhydrous Na₂SO₄. After filtration, the filtrate was concentrated under reduced pressure. The residue was purified by Prep-TLC (DCM/MeOH 12:1) to afford 730 mg (75%) of 3-bromo-6-cyclopropylpyrazolo[1,5-a]pyridine as a light yellow oil. ¹H NMR (400 MHZ, DMSO-d₆) δ (ppm) 8.57 (s, 1H), 8.06 (s, 1H), 7.55-7.47 (m, 1H), 7.10-7.07 (m, 1H), 2.08-1.94 (m, 1H), 1.03-0.87 (m, 2H), 0.86-0.70 (m, 2H). MS (ESI) m/z [M+H]⁺ calcd. for C₁₀H₉BrN₂, 237.0; found, 237.1.

### Step 3. tert-butyl 4-{6-cyclopropylpyrazolo[1,5-a]pyridin-3-yl}piperazine-1-carboxylate (Compound 213)

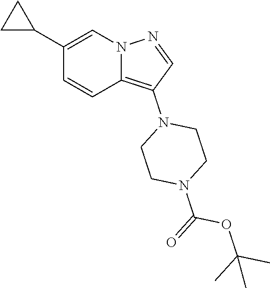 [SMILES: CC(C)(C)OC(=O)N1CCN(c2cnn3cc(C4CC4)ccc23)CC1]

To a stirred solution of 3-bromo-6-cyclopropylpyrazolo[1,5-a]pyridine (350 mg, 1.48 mmol, as prepared in the previous step) and tert-butyl piperazine-1-carboxylate (412.4 mg, 2.21 mmol) in tBuOH (10 mL) dioxane (5 mL) was added KOtBu (496.9 mg, 4.43 mmol), tBuXPhos Pd G1 (50 mg, 0.0296 mmol) at rt under nitrogen. The reaction was stirred overnight at 80° C. under nitrogen then concentrated under vacuum. To the resulting mixture was added H₂O (50 mL), which was extracted with EtOAc (2×100 mL). The combined organic layers were washed with brine (2×5 mL) and dried over anhydrous Na₂SO₄. After filtration, the filtrate was concentrated under reduced pressure. The residue was purified by Prep-TLC (PE/EtOAc 1:1) to afford crude product. The crude product was purified by Prep-HPLC to afford 165.6 mg (33%) of tert-butyl 4-{6-cyclopropylpyrazolo[1,5-a]pyridin-3-yl}piperazine-1-carboxylate (Compound 213) as a white solid. ¹H NMR (400 MHZ, DMSO-d₆) δ (ppm) 8.35 (s, 1H), 7.71 (s, 1H), 7.52 (d, J=9.2 Hz, 1H), 6.80-6.82 (m, 1H), 3.49 (m, 4H), 2.90 (m, 4H), 1.91-1.97 (m, 1H), 1.43 (s, 9H), 0.97-0.85 (m, 2H), 0.79-0.66 (m, 2H). MS (ESI) m/z [M+H]⁺ calcd. for C₁₉H₂₆N₄O₂, 343.2; found, 343.1. HPLC Purity: 254 nm: 99.2%.

### Example 157: Synthesis of Exemplary Compound 214

### tert-butyl 4-(6-cyclopentylpyrazolo[1,5-a]pyridin-3-yl)piperazine-1-carboxylate (Compound 214)

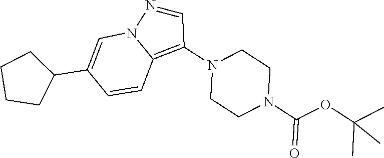 [SMILES: CC(C)(C)OC(=O)N1CCN(c2cnn3cc(C4CCCC4)ccc23)CC1]

### Step 1. Preparation of 6-(cyclopent-1-en-1-yl)pyrazolo[1,5-a]pyridine

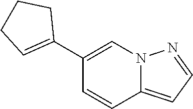 [SMILES: C1=C(c2ccc3ccnn3c2)CCC1]

To a solution of 6-bromopyrazolo[1,5-a]pyridine (1.00 g, 5.08 mmol) and cyclopent-1-en-1-ylboronic acid (0.57 g, 5.08 mmol) in dioxane (10.0 mL) and H₂O (1.0 mL) was added XPhos Pd G3 (0.43 g, 0.51 mmol), XPhos (0.24 g, 0.51 mmol), and K₃PO₄ (2.15 g, 10.2 mmol) under nitrogen. The reaction was stirred for 16 h at 90° C. under nitrogen then concentrated under reduced pressure. The residue was purified by silica gel column chromatography eluting with PE/EtOAc (10:1) to afford 1.0 g (96%) of 6-(cyclopent-1-en-1-yl)pyrazolo[1,5-a]pyridine as a yellow solid. ¹H NMR (400 MHZ, DMSO-d₆) δ (ppm) 8.52 (s, 1H), 7.97 (d, J=2.2 Hz, 1H), 7.65 (d, J=9.2 Hz, 1H), 7.53 (d, J=9.4 Hz, 1H), 6.61-6.56 (m, 1H), 6.43-6.37 (m, 1H), 2.75-2.66 (m, 2H), 2.56-2.51 (m, 2H), 2.05-1.93 (m, 2H). MS (ESI) m/z [M+H]⁺ calcd. for C₁₂H₁₂N₂, 185.2; found, 185.3.

### Step 2. Preparation of 6-cyclopentylpyrazolo[1,5-a]pyridine

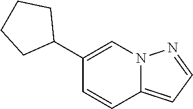 [SMILES: c1cc2ccc(C3CCCC3)cn2n1]

To a solution of 6-(cyclopent-1-en-1-yl)pyrazolo[1,5-a]pyridine (1 g, 5.43 mmol, as prepared in the previous step) in MeOH (20 mL) was added Pd/C (289 mg, 2.71 mmol). The reaction was stirred for 1 h at rt under a hydrogen atmosphere. The resulting mixture was filtered, the filter cake was washed with MeOH (2×2 mL). The filtrate was concentrated under reduced pressure. The residue was purified by reversed-phase flash chromatography with C18 silica gel column eluting with 10% to 100 ACN in water (10 mM NH₄HCO₃) to afford 380 mg (34%) of 6-cyclopentylpyrazolo[1,5-a]pyridine as a white solid. ¹H NMR (400 MHz, DMSO-d₆) δ (ppm) 8.49 (s, 1H), 7.91 (d, J=2.2 Hz, 1H), 7.68-7.59 (m, 1H), 7.17 (dd, J=9.1, 1.5 Hz, 1H), 6.53 (d, J=2.2 Hz, 1H), 3.08-2.95 (m, 1H), 2.10-1.92 (m, 2H), 1.85-1.72 (m, 2H), 1.72-1.50 (m, 4H). MS (ESI) m/z [M+H]⁺ calcd. for C₁₂H₁₄N₂, 187.1; found, 187.2.

### Step 3. Preparation of 3-bromo-6-cyclopentylpyrazolo[1,5-a]pyridine

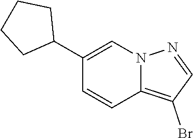 [SMILES: Brc1cnn2cc(C3CCCC3)ccc12]

To a solution of 6-cyclopentylpyrazolo[1,5-a]pyridine (388 mg, 2.08 mmol, as prepared in the previous step) in DCM (5.0 mL) was added NBS (408 mg, 2.29 mmol). The reaction was stirred for 1 h at rt then concentrated under reduced pressure. The residue was purified by silica gel column chromatography eluting with PE/EtOAc (9:1) to afford 450 mg (73%) of 3-bromo-6-cyclopentylpyrazolo[1,5-a]pyridine as a yellow oil. ¹H NMR (400 MHZ, DMSO-d₆) δ (ppm) 8.54 (s, 1H), 8.07 (s, 1H), 7.51 (d, J=9.2 Hz, 1H), 7.30 (dd, J=9.1, 1.5 Hz, 1H), 3.11-2.90 (m, 1H), 2.13-1.96 (m, 2H), 1.84-1.50 (m, 6H). MS (ESI) m/z [M+H]⁺ calcd. for C₁₂H₁₃BrN₂, 265.0; found, 265.1.

### Step 4. Preparation of tert-butyl 4-(6-cyclopentylpyrazolo[1,5-a]pyridin-3-yl)piperazine-1-carboxylate (Compound 214)

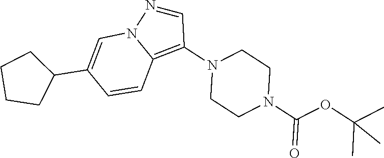 [SMILES: CC(C)(C)OC(=O)N1CCN(c2cnn3cc(C4CCCC4)ccc23)CC1]

To a solution of 3-bromo-6-cyclopentylpyrazolo[1,5-a]pyridine (420 mg, 1.58 mmol, as prepared in the previous step) and tert-butyl piperazine-1-carboxylate (354 mg, 1.90 mmol) in tBuOH (5.5 mL) and dioxane (1.5 mL) was added with KOtBu (267 mg, 2.38 mmol) and tBuXPhos Pd G1 (163 mg, 0.24 mmol) under nitrogen. The reaction was stirred for 16 h at 90° C. under nitrogen then concentrated under reduced pressure. The residue was purified by silica gel column chromatography eluting with PE/EtOAc (5:1). The crude product was purified by Prep-HPLC to afford 98.9 mg (17%) of tert-butyl 4-(6-cyclopentylpyrazolo[1,5-a]pyridin-3-yl)piperazine-1-carboxylate (Compound 214) as a yellow solid. ¹H NMR (400 MHz, DMSO-d₆) δ (ppm) 8.32 (s, 1H), 7.72 (s, 1H), 7.56 (dd, J=9.2, 0.9 Hz, 1H), 7.04 (dd, J=9.3, 1.5 Hz, 1H), 3.50 (t, J=4.9 Hz, 4H), 3.05-2.95 (m, 1H), 2.94-2.88 (m, 4H), 2.08-1.93 (m, 2H), 1.84-1.72 (m, 2H), 1.71-1.62 (m, 2H), 1.61-1.49 (m, 2H), 1.43 (s, 9H) MS (ESI) m/z [M+H]⁺ calcd. for C₂₁H₃₀N₄O₂, 371.2; found, 371.2; HPLC purity: 254 nm: 99.3%.

### Example 158: Synthesis of Exemplary Compound 215

### tert-butyl 4-[6-(3-hydroxycyclopentyl)pyrazolo[1,5-a]pyridin-3-yl]piperazine-1-carboxylate (Compound 215)

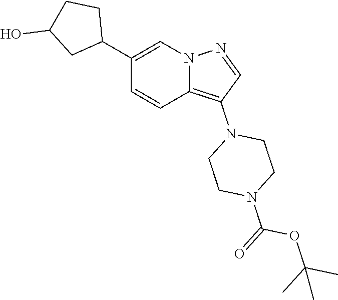 [SMILES: CC(C)(C)OC(=O)N1CCN(c2cnn3cc(C4CCC(O)C4)ccc23)CC1]

### Step 1. Synthesis of 3-{pyrazolo[1,5-a]pyridin-6-yl}cyclopent-2-en-1-one

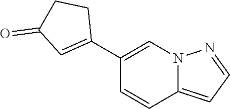 [SMILES: O=C1C=C(c2ccc3ccnn3c2)CC1]

To a stirred solution of 6-bromopyrazolo[1,5-a]pyridine (1.5 g, 7.61 mmol) and 3-(4,4,5-trimethyl-1,3,2-dioxaborolan-2-yl)cyclopent-2-en-1-one (1.77 g, 9.14 mmol) in dioxane (20 mL) and H₂O (2 mL) were added Pd(dppf)Cl₂ (111.4 mg, 0.15 mmol), Cs₂CO₃ (4.96 g, 15.23 mmol) in portions at rt. The reaction was stirred overnight at 80° C. under nitrogen then was concentrated under vacuum. To the resulting mixture was added H₂O (50 mL), which was extracted with EtOAc (2×100 mL). The combined organic layers were washed with brine (2×10 mL) and dried over anhydrous Na₂SO₄. After filtration, the filtrate was concentrated under reduced pressure. The residue was purified by silica gel column chromatography eluting with PE/EtOAc (1:1) to afford 1.6 g (106%) of 3-{pyrazolo[1,5-a]pyridin-6-yl}cyclopent-2-en-1-one as a light brown solid. ¹H NMR (400 MHZ, DMSO-d₆) δ (ppm) 9.17 (s, 1H), 8.14 (d, J=2.2 Hz, 1H), 7.77 (d, J=9.3 Hz, 1H), 7.66 (m, 1H), 6.94-6.63 (m, 2H), 3.10-3.08 (m, 2H), 2.48 (m, 2H). MS (ESI) m/z [M+H]⁺ calcd. for C₁₂H₁₀N₂O, 199.1 found, 199.2.

### Step 2. Synthesis of 3-{pyrazolo[1,5-a]pyridin-6-yl}cyclopentan-1-ol

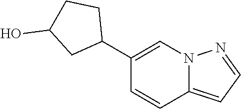 [SMILES: OC1CCC(c2ccc3ccnn3c2)C1]

To a stirred solution of 3-{pyrazolo[1,5-a]pyridin-6-yl}cyclopent-2-en-1-one (1 g, 5.05 mmol, as prepared in the previous step) in THF (20 mL) was added LiAlH₄ (287.2 mg, 7.57 mmol) in portions at 0° C. The reaction was stirred for 2 h at rt. The reaction was quenched by the addition of an ice/water mixture (10 mL) at 0° C. To the above mixture was added 10 ml 20% NaOH solution at 0° C. Then resulting mixture was filtered, and the filter cake was washed with EtOAc (2×10 mL). The filtrate was concentrated under reduced pressure. The crude product was purified by Prep-HPLC to afford 240 mg (24%) of 3-{pyrazolo[1,5-a]pyridin-6-yl}cyclopentan-1-ol as a white solid. ¹H NMR (400 MHZ, DMSO-d₆) δ 8.49 (s, 1H), 7.91 (d, J=2.3 Hz, 1H), 7.64 (m, 1H), 7.25-7.14 (m, 1H), 6.53 (d, J=2.2 Hz, 1H), 4.78-4.55 (m, 1H), 4.40-4.17 (m, 1H), 3.17-2.95 (m, 1H), 2.32-2.22 (m, 1H), 2.20-1.87 (m, 2H), 1.83-1.64 (m, 2H), 1.55-1.52 (m, 2H). MS (ESI) m/z [M+H]⁺ calcd. for C₁₂H₁₄N₂O, 203.1; found, 203.2.

### Step 3. Synthesis of 3-{3-bromopyrazolo[1,5-a]pyridin-6-yl}cyclopentan-1-ol

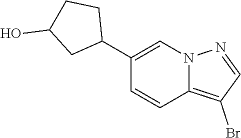 [SMILES: OC1CCC(c2ccc3c(Br)cnn3c2)C1]

To a stirred solution of 3-{pyrazolo[1,5-a]pyridin-6-yl}cyclopentan-1-ol (200 mg, 0.99 mmol, as prepared in the previous step) in DCM (20 mL) was added NBS (211.2 mg, 1.19 mmol) in portions at rt. The reaction was stirred for 1 h at rt then quenched with sat. aq. sodium thiosulfate at 0° C. The mixture was diluted in water (100 mL), then was extracted with DCM (2×200 mL). The combined organic layers were washed with brine (2×5 mL) and dried over anhydrous Na₂SO₄. After filtration, the filtrate was concentrated under reduced pressure. The residue was purified by silica gel column chromatography eluting with PE/EtOAc (1:1) to afford 220 mg (79%) of 3-{3-bromopyrazolo[1,5-a]pyridin-6-yl}cyclopentan-1-ol as a light yellow oil. ¹H NMR (400 MHZ, DMSO-d₆) δ (ppm) 8.57 (s, 1H), 8.08 (s, 1H), 7.54 (m, 1H), 7.41-7.30 (m, 1H), 4.73-4.67 (m, 1H), 4.28-4.23 (m, 1H), 3.15 (m, 2H), 2.29 (m, 1H), 2.22-1.88 (m, 3H), 1.83-1.66 (m, 2H), 1.63-1.50 (m, 2H), 1.41-1.20 (m, 1H). MS (ESI) m/z [M+H]⁺ calcd. for C₁₂H₁₃BrN₂O, 281.0; found, 281.1.

### Step 4. tert-butyl 4-[6-(3-hydroxycyclopentyl)pyrazolo[1,5-a]pyridin-3-yl]piperazine-1-carboxylate (Compound 215)

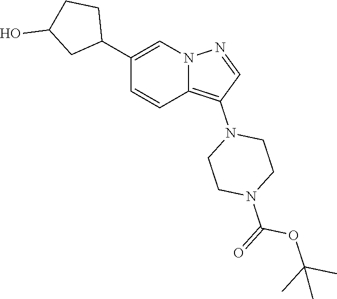 [SMILES: CC(C)(C)OC(=O)N1CCN(c2cnn3cc(C4CCC(O)C4)ccc23)CC1]

To a stirred solution of 3-{3-bromopyrazolo[1,5-a]pyridin-6-yl}cyclopentan-1-ol (80 mg, 0.29 mmol, as prepared in the previous step) and tert-butyl piperazine-1-carboxylate (265 mg, 1.43 mmol) in tBuOH (10 mL) and dioxane (5 mL) was added KOtBu (95.8 mg, 0.85 mmol), tBuXPhos Pd G1 (19.5 mg, 0.028 mmol) at rt. The reaction was stirred overnight at 80° C. under nitrogen then concentrated under vacuum. To the resulting mixture was added H₂O (50 mL), which was extracted with EtOAc (2×100 mL). The combined organic layers were washed with brine (2×10 mL) and dried over anhydrous Na₂SO₄. After filtration, the filtrate was concentrated under reduced pressure. The residue was purified by reversed-phase flash chromatography with C₁₈ silica gel column eluting with 10% to 80% ACN in water (10 mM NH₄HCO₃). The crude product was further purified by Prep-HPLC to afford 14.8 mg (13%) of tert-butyl 4-[6-(3-hydroxycyclopentyl)pyrazolo[1,5-a]pyridin-3-yl]piperazine-1-carboxylate (Compound 215) as a white solid. ¹H NMR (400 MHZ, DMSO-d₆) δ (ppm) 8.33 (s, 1H), 7.72 (s, 1H), 7.57 (m, 1H), 7.15-7.01 (m, 1H), 4.71-4.58 (m, 1H), 4.30-4.23 (m, 1H), 3.50 (m, 4H), 2.91 (m, 4H), 2.39-2.20 (m, 1H), 2.19-2.00 (m, 1H), 1.91 (m, 1H), 1.80-1.73 (m, 2H), 1.63-1.52 (m, 2H), 1.43 (s, 9H). MS (ESI) m/z [M+H]⁺ calcd. for C₂₁H₃₀N₄O₃, 387.2; found, 387.2, HPLC Purity: 254 nm: 96.4%.

### Example 159: Synthesis of Exemplary Compound 216

### tert-butyl 4-[6-(pyridin-2-yl)pyrazolo[1,5-a]pyridin-3-yl]piperazine-1-carboxylate (Compound 216)

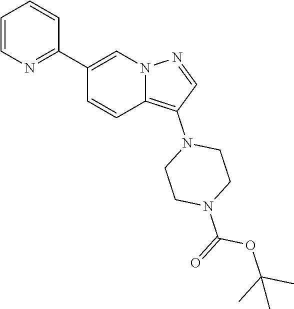 [SMILES: CC(C)(C)OC(=O)N1CCN(c2cnn3cc(-c4ccccn4)ccc23)CC1]

### Step 1. Preparation of 6-(pyridin-2-yl)pyrazolo[1,5-a]pyridine

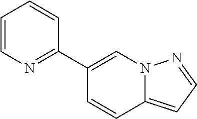 [SMILES: c1ccc(-c2ccc3ccnn3c2)nc1]

To a solution of 6-bromopyrazolo[1,5-a]pyridine (1.00 g, 5.08 mmol) in dioxane (40 mL) was added Pd(PPh₃)₄ (879.7 mg, 0.76 mmol). To the mixture was added 2-(tributylstannyl)pyridine (2.24 g, 6.09 mmol) dropwise at rt under nitrogen. The reaction stirred for 2 h at 90° C. then was allowed to cool to rt. The reaction was quenched by the addition of KF (aq., 3 mL) at rt. The resulting mixture was concentrated under reduced pressure. The residue was purified by silica gel column chromatography eluting with PE/EtOAc (2:1) to afford 530 mg (54%) of 6-(pyridin-2-yl)pyrazolo[1,5-a]pyridine as a white solid. ¹H NMR (400 MHz, DMSO-d₆) δ (ppm) 9.50-9.26 (s, 1H), 8.69 (t, J=5.0 Hz, 1H), 8.25-7.68 (m, 5H), 7.38 (d, J=10.6 Hz, 1H), 6.79-6.51 (m, 1H). MS (ESI) m/z [M+H]⁺ calcd. for C₁₂H₉N₃, 196.0; found, 196.0.

### Step 2. Preparation of 3-bromo-6-(pyridin-2-yl)pyrazolo[1,5-a]pyridine

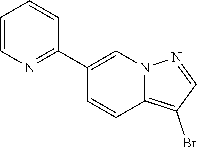 [SMILES: Brc1cnn2cc(-c3ccccn3)ccc12]

To a solution of 6-(pyridin-2-yl)pyrazolo[1,5-a]pyridine (500 mg, 2.56 mmol, as prepared in the previous step) in DCM (10 mL) was added NBS (501.4 mg, 2.82 mmol). The reaction was stirred for 1 h at rt under nitrogen then was concentrated under reduced pressure. The residue was dissolved in water (100 mL) then extracted with EtOAc (3×100 mL) and dried over anhydrous Na₂SO₄. After filtration, the filtrate was concentrated under reduced pressure. The residue was purified by Prep-TLC (PE/EtOAc 3:1) to afford 600 mg (81%) of 3-bromo-6-(pyridin-2-yl)pyrazolo[1,5-a]pyridine as a white solid. ¹H NMR (400 MHZ, DMSO-d₆) δ (ppm) 9.45 (s, 1H), 8.70 (d, J=4.9 Hz, 1H), 8.26 (d, J=3.0 Hz, 1H), 8.21-8.09 (m, 2H), 7.93 (t, J=7.8 Hz, 1H), 7.71 (d, J=9.5 Hz, 1H), 7.41 (d, J=7.6 Hz, 1H). MS (ESI) m/z [M+H]⁺ calcd. for C₁₂H₈BrN₃, 274.1; found, 274.1.

### Step 3. tert-butyl 4-[6-(pyridin-2-yl)pyrazolo[1,5-a]pyridin-3-yl]piperazine-1-carboxylate (Compound 216)

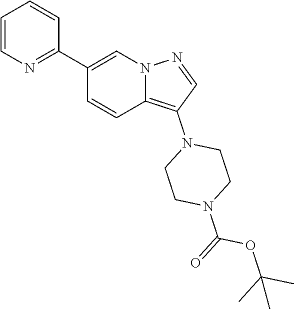 [SMILES: CC(C)(C)OC(=O)N1CCN(c2cnn3cc(-c4ccccn4)ccc23)CC1]

To a stirred solution of 3-bromo-6-(pyridin-2-yl)pyrazolo[1,5-a]pyridine (150 mg, 0.55 mmol, as prepared in the previous step) and tert-butyl piperazine-1-carboxylate (509.6 mg, 2.74 mmol) in dioxane (8 mL) and tBuOH (8 mL) was added KOtBu (122.8 mg, 1.09 mmol) and tBuXPhos Pd G1 (56.4 mg, 0.082 mmol) at rt under nitrogen. The reaction was stirred overnight at 90° C. then allowed to cool to rt. The resulting mixture was concentrated under reduced pressure, extracted with EtOAc (3×100 mL), and dried over anhydrous Na₂SO₄. After filtration, the filtrate was concentrated under reduced pressure. The crude product was purified by reverse phase flash with YMC-Actus Triart C18 column eluting with 45% to 75% ACN in water (10 mM NH₄HCO₃) to afford 89.7 mg (43%) of tert-butyl 4-[6-(pyridin-2-yl)pyrazolo[1,5-a]pyridin-3-yl]piperazine-1-carboxylate (Compound 216) as a yellow solid. ¹H NMR (400 MHZ, DMSO-d₆) δ (ppm) 9.25 (s, 1H), 8.67 (d, J=4.8, Hz, 1H), 8.09 (d, J=8.1 Hz, 1H), 7.94-7.82 (m, 3H), 7.75 (d, J=9.4, 1H), 7.39-7.30 (m, 1H), 3.53 (t, J=4.8 Hz, 4H), 2.98 (t, J=5.0 Hz, 4H), 1.44 (s, 9H). MS (ESI) m/z [M+H]⁺ calcd. for C₂₁H₂₅N₅O₂, 380.2; found, 380.2; HPLC purity: 254 nm: 99.8%.

### Example 160: Synthesis of Exemplary Compound 217

### tert-butyl 4-(6-(pyridin-3-yl)pyrazolo[1,5-a]pyridin-3-yl)piperazine-1-carboxylate (Compound 217)

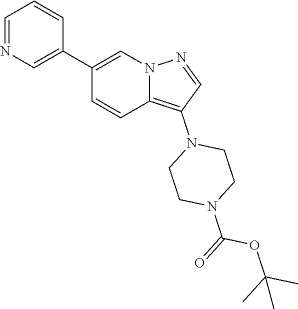 [SMILES: CC(C)(C)OC(=O)N1CCN(c2cnn3cc(-c4cccnc4)ccc23)CC1]

### Step 1. Preparation of 6-(pyridin-3-yl)pyrazolo[1,5-a]pyridine

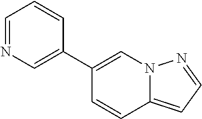 [SMILES: c1cncc(-c2ccc3ccnn3c2)c1]

To a solution of 6-bromopyrazolo[1,5-a]pyridine (500 mg, 2.54 mmol) and pyridin-3-ylboronic acid (374.3 mg, 3.05 mmol) in dioxane (5 mL) and H₂O (0.5 mL) were added K₂CO₃ (1.05 g, 7.61 mmol) and Pd(dppf)Cl₂ (371.3 mg, 0.51 mmol). The reaction was stirred for 16 h at 90° C. under nitrogen. The mixture was cooled to rt then concentrated under reduced pressure. The residue was diluted with water and extracted with EtOAc (3×50 mL). The combined organic layers were washed with brine (10 mL), dried over anhydrous Na₂SO₄. After filtration, the filtrate was concentrated under reduced pressure. The residue was purified by silica gel column chromatography eluting with DCM/MeOH (10:1) to afford 300 mg (61%) of 6-(pyridin-3-yl)pyrazolo[1,5-a]pyridine as a light yellow solid. ¹H NMR (400 MHZ, DMSO-d₆) δ (ppm) 9.18 (s, 1H), 9.04-8.99 (m, 1H), 8.60 (dd, J=4.9, 1.6 Hz, 1H), 8.24-8.17 (m, 1H), 8.07 (d, J=2.3 Hz, 1H), 7.84 (d, J=9.3 Hz, 1H), 7.63 (dd, J=9.2, 1.7 Hz, 1H), 7.56-7.48 (m, 1H), 6.68 (d, J=2.3 Hz, 1H). MS (ESI) m/z [M+H]⁺ calcd. for C₁₂H₉N₃, 196.1; found, 196.2.

### Step 2. Preparation of 3-bromo-6-(pyridin-3-yl)pyrazolo[1,5-a]pyridine

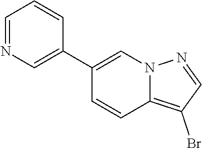 [SMILES: Brc1cnn2cc(-c3cccnc3)ccc12]

To a solution of 6-(pyridin-3-yl)pyrazolo[1,5-a]pyridine (600 mg, 3.07 mmol, as prepared in the previous step) in DCM (5 mL) was added NBS (656.4 mg, 3.69 mmol). The reaction was stirred for 1 h at rt under nitrogen, and the resulting mixture was concentrated under reduced pressure. The residue was diluted with water and extracted with EtOAc (3×50 mL). The combined organic layers were washed with brine (10 mL), dried over anhydrous Na₂SO₄. After filtration, the filtrate was concentrated under reduced pressure. The residue was purified by Prep-TLC (DCM/MeOH 10:1) to afford 700 mg (83%) of 3-bromo-6-(pyridin-3-yl)pyrazolo[1,5-a]pyridine as a yellow solid. ¹H NMR (400 MHZ, DMSO-d₆) δ (ppm) 9.24 (s, 1H), 9.02 (d, J=2.4 Hz, 1H), 8.62 (dd, J=4.7, 1.6 Hz, 1H), 8.26-8.18 (m, 2H), 7.82-7.67 (m, 2H), 7.56-7.48 (m, 1H). MS (ESI) m/z [M+H]⁺ calcd. for C₁₂H₈BrN₃, 274.0; found, 274.1.

### Step 3. tert-butyl 4-(6-(pyridin-3-yl)pyrazolo[1,5-a]pyridin-3-yl)piperazine-1-carboxylate (Compound 217)

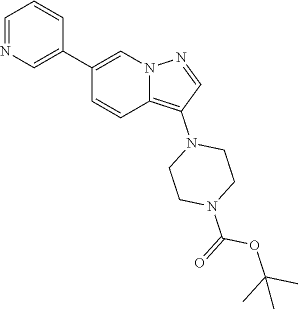 [SMILES: CC(C)(C)OC(=O)N1CCN(c2cnn3cc(-c4cccnc4)ccc23)CC1]

Into a 40 mL vial were added 3-bromo-6-(pyridin-3-yl)pyrazolo[1,5-a]pyridine (0.8 g, 2.92 mmol, as prepared in the previous step), tBuOH (10 mL) and dioxane (10 mL). To the solution were added KOtBu (654.9 mg, 5.84 mmol), tBuXPhos Pd G1 (300.6 mg, 0.44 mmol) and tert-butyl piperazine-1-carboxylate (2.72 g, 14.59 mmol) at rt under nitrogen. The reaction was stirred for 8 h at 90° C. under nitrogen then diluted with water (50 mL). The resulting mixture was extracted with EtOAc (3×200 mL). The combined organic layers were washed with brine (10 mL) and dried over anhydrous Na₂SO₄. After filtration, the filtrate was concentrated under reduced pressure. The residue was purified by silica gel column chromatography eluting with PE/EtOAc (9:1) to afford crude product. The crude product was purified by Prep-HPLC to afford 142.6 mg (13%) of tert-butyl 4-(6-(pyridin-3-yl)pyrazolo[1,5-a]pyridin-3-yl)piperazine-1-carboxylate (Compound 217) as a light yellow solid. ¹H NMR (400 MHz, DMSO-d₆) δ (ppm) 9.05-8.97 (m, 2H), 8.59 (dd, J=4.8, 1.6 Hz, 1H), 8.22-8.15 (m, 1H), 7.90 (s, 1H), 7.77 (d, J=9.3 Hz, 1H), 7.54-7.45 (m, 2H), 3.53 (t, J=2.0 Hz, 4H), 2.98 (t, J=5.0 Hz, 4H), 1.44 (s, 9H). MS (ESI) m/z [M+H]⁺ calcd. for C₂₁H₂₅N₅O₂, 380.2; found, 380.1; HPLC purity: 254 nm: 99.0%.

### Example 161: Synthesis of Exemplary Compound 218

### tert-butyl 4-(6-(pyridin-4-yl)pyrazolo[1,5-a]pyridin-3-yl)piperazine-1-carboxylate (Compound 218)

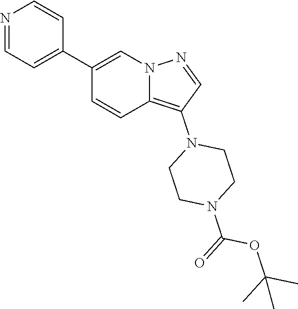 [SMILES: CC(C)(C)OC(=O)N1CCN(c2cnn3cc(-c4ccncc4)ccc23)CC1]

### Step 1. Preparation of 6-(pyridin-4-yl)pyrazolo[1,5-a]pyridine

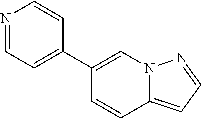 [SMILES: c1cc(-c2ccc3ccnn3c2)ccn1]

To a stirred solution of 6-bromopyrazolo[1,5-a]pyridine (1.5 g, 7.61 mmol) and pyridin-4-ylboronic acid (1.11 g, 9.06 mmol) in dioxane (60 mL) and H₂O (6 mL) were added Pd(dppf)Cl₂ (1.11 g, 1.52 mmol) and K₂CO₃ (2.10 g, 15.23 mmol) in portions at rt under nitrogen. The reaction was stirred overnight at 90° C. The mixture was allowed to cool to rt then concentrated under reduced pressure. The residue was dissolved in water (100 mL), extracted with EtOAc (4×100 mL) and dried over anhydrous Na₂SO₄. After filtration, the filtrate was concentrated under reduced pressure. The residue was purified by silica gel column chromatography eluting with PE/EtOAc (1:1) to afford 600 mg (40%) of 6-(pyridin-4-yl)pyrazolo[1,5-a]pyridine as a brown solid. ¹H NMR (400 MHZ, DMSO-d₆) δ (ppm) 9.31 (s, 1H), 8.69-8.63 (m, 2H), 8.11 (d, J=2.3 Hz, 1H), 7.89-7.82 (m, 3H), 7.69 (d, J=9.3 Hz, 1H), 6.70 (d, J=2.2 Hz, 1H). MS (ESI) m/z [M+H]⁺ calcd. for C₁₂H₉N₃, 196.0; found, 196.0.

### Step 2. Preparation of 3-bromo-6-(pyridin-4-yl)pyrazolo[1,5-a]pyridine

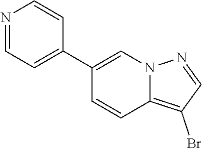 [SMILES: Brc1cnn2cc(-c3ccncc3)ccc12]

To a solution of 6-(pyridin-4-yl)pyrazolo[1,5-a]pyridine (500 mg, 2.56 mmol, as prepared in the previous step) in DCM (10 mL) were added NBS (501.4 mg, 2.82 mmol). The reaction was stirred for 1 h at rt under nitrogen then concentrated under reduced pressure. The residue was dissolved in water (100 mL), extracted with EtOAc (3×100 mL) and dried over anhydrous Na₂SO₄. After filtration, the filtrate was concentrated under reduced pressure. The residue was purified by Prep-TLC (PE/EtOAc 3:1) to afford 600 mg (81%) of 3-bromo-6-(pyridin-4-yl)pyrazolo[1,5-a]pyridine as a yellow solid. ¹H NMR (400 MHZ, DMSO-d₆) δ (ppm) 9.37 (s, 1H), 8.67 (d, J=4.5 Hz, 2H), 8.28 (d, J=1.9 Hz, 1H), 7.90-7.82 (m, 3H), 7.73 (d, J=9.2 Hz, 1H). MS (ESI) m/z [M+H]⁺ calcd. for C₁₂H₈BrN₃, 274.1; found, 274.1.

### Step 3. tert-butyl 4-(6-(pyridin-4-yl)pyrazolo[1,5-a]pyridin-3-yl)piperazine-1-carboxylate (Compound 218)

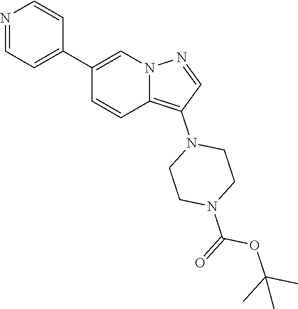 [SMILES: CC(C)(C)OC(=O)N1CCN(c2cnn3cc(-c4ccncc4)ccc23)CC1]

To a stirred mixture of 3-bromo-6-(pyridin-4-yl)pyrazolo[1,5-a]pyridine (600 mg, 2.19 mmol, as prepared in the previous step) and tert-butyl piperazine-1-carboxylate (2.04 g, 10.95 mmol) in dioxane (24 mL) and tBuOH (24 mL) were added KOtBu (491.2 mg, 4.38 mmol) and tBuXPhos Pd G1 (225.5 mg, 0.33 mmol) in portions at rt under nitrogen. The reaction was stirred for overnight at 90° C. The mixture was allowed to cool to rt then concentrated under reduced pressure. The residue was diluted with water, extracted with EtOAc (3×100 mL) and dried over anhydrous Na₂SO₄. After filtration, the filtrate was concentrated under reduced pressure. The crude product was purified by reverse phase flash chromatography to afford 165.8 mg (20%) of tert-butyl 4-(6-(pyridin-4-yl)pyrazolo[1,5-a]pyridin-3-yl)piperazine-1-carboxylate (Compound 218) as a yellow solid. ¹H NMR (400 MHz, DMSO-d₆) δ (ppm) 9.16 (s, 1H), 8.65 (s, 2H), 8.01-7.72 (m, 4H), 7.54 (t, J=8.1 Hz, 1H), 3.42-3.22 (m, 4H), 3.05-2.90 (m, 4H), 1.43 (t, J=5.4 Hz, 9H). MS (ESI) m/z [M+H]⁺ calcd. for C₂₁H₂₅NsO₂, 380.2; found, 380.2; HPLC purity: 254 nm: 99.9%.

### Example 162: Synthesis of Exemplary Compound 219

### tert-butyl 4-(6-(pyrimidin-5-yl)pyrazolo[1,5-a]pyridin-3-yl)piperazine-1-carboxylate (Compound 219)

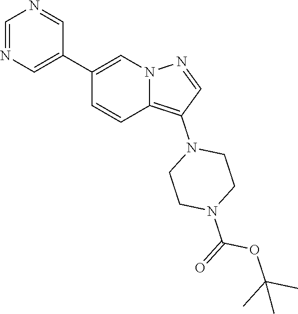 [SMILES: CC(C)(C)OC(=O)N1CCN(c2cnn3cc(-c4cncnc4)ccc23)CC1]

### Step 1. Preparation of 6-(pyrimidin-5-yl)pyrazolo[1,5-a]pyridine

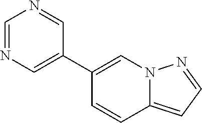 [SMILES: c1ncc(-c2ccc3ccnn3c2)cn1]

To a solution of 6-bromopyrazolo[1,5-a]pyridine (2.0 g, 10.17 mmol) and pyrimidin-5-ylboronic acid (1.26 g, 10.17 mmol) in dioxane (10.0 mL) and H₂O (1.0 mL) was added Pd(dppf)Cl₂·DCM (0.83 g, 1.02 mmol) and Cs₂CO₃ (6.61 g, 20.29 mmol) under nitrogen. The reaction was stirred for 2 h at 80° C. under nitrogen then concentrated under reduced pressure. The residue was purified by silica gel column chromatography eluting with PE/EtOAc (1:1) to afford 1.273 g (58%) of 6-(pyrimidin-5-yl)pyrazolo[1,5-a]pyridine as a brown solid. ¹H NMR (400 MHz, DMSO-d₆) δ (ppm) 9.32 (s, 1H), 9.26 (s, 2H), 9.21 (s, 1H), 8.10 (d, J=2.2 Hz, 1H), 7.87 (dd, J=9.3, 0.9 Hz, 1H), 7.69 (dd, J=9.2, 1.7 Hz, 1H), 6.71 (dd, J=2.3, 0.9 Hz, 1H). MS (ESI) m/z [M+H]⁺ calcd. for C₁₁H₈N₄, 197.1; found, 197.2.

### Step 2. Preparation of 3-bromo-6-(pyrimidin-5-yl)pyrazolo[1,5-a]pyridine

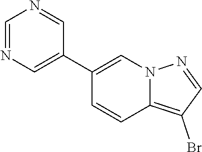 [SMILES: Brc1cnn2cc(-c3cncnc3)ccc12]

To a solution of 6-(pyrimidin-5-yl)pyrazolo[1,5-a]pyridine (1.25 g, 6.37 mmol, as prepared in the previous step) in DCM (30 mL) was added NBS (1.25 g, 7.02 mmol). The reaction was stirred for 2 h at rt. The precipitated solids were collected by filtration and washed with DCM (2×10 mL). The solids were dried under vacuum to afford 0.6 g (29%) of 3-bromo-6-(pyrimidin-5-yl)pyrazolo[1,5-a]pyridine as a white solid. ¹H NMR (400 MHZ, DMSO-d₆) (ppm) δ (ppm) 9.38 (s, 1H), 9.27 (s, 2H), 9.23 (s, 1H), 8.28 (s, 1H), 7.85 (dd, J=9.2, 1.6 Hz, 1H), 7.75 (d, J=9.2 Hz, 1H). MS (ESI) m/z [M+H]⁺ calcd. for C₁₁H₇BrN₄, 275.0; found, 275.1.

### Step 3. Preparation of tert-butyl 4-(6-(pyrimidin-5-yl)pyrazolo[1,5-a]pyridin-3-yl)piperazine-1-carboxylate (Compound 219)

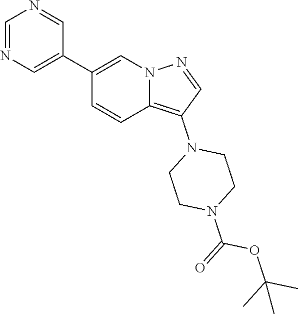 [SMILES: CC(C)(C)OC(=O)N1CCN(c2cnn3cc(-c4cncnc4)ccc23)CC1]

To a solution of 3-bromo-6-(pyrimidin-5-yl)pyrazolo[1,5-a]pyridine (560 mg, 2.04 mmol, as prepared in the previous step) and tert-butyl piperazine-1-carboxylate (455 mg, 2.44 mmol) in tBuOH (8.0 mL) and dioxane (4.0 mL) was added with KOtBu (343 mg, 3.05 mmol) and tBuXPhos Pd G1 (210 mg, 0.31 mmol) under nitrogen. The reaction was stirred for 20 h at 90° C. under nitrogen then concentrated under reduced pressure. The residue was purified by silica gel column chromatography eluting with PE/EtOAc (1:3). The crude product was further purified by Prep-HPLC to afford 49.7 mg (6%) of tert-butyl 4-(6-(pyrimidin-5-yl)pyrazolo[1,5-a]pyridin-3-yl)piperazine-1-carboxylate (Compound 219) as a yellow green solid. ¹H NMR (400 MHZ, DMSO-d₆) δ (ppm) 9.25 (s, 2H), 9.22-9.14 (m, 2H), 7.93 (s, 1H), 7.81 (d, J=9.3 Hz, 1H), 7.55 (d, J=9.4 Hz, 1H), 3.53 (t, J=4.9 Hz, 4H), 2.99 (t, J=5.0 Hz, 4H), 1.44 (s, 9H). MS (ESI) m/z [M+H]⁺ calcd. for C₂₀H₂₄N₆O₂, 381.2; found, 381.2; HPLC purity: 254 nm: 98.5%.

### Example 163: Synthesis of Exemplary Compound 220

### tert-butyl 4-(6-(1,3-dimethyl-1H-pyrazol-4-yl)pyrazolo[1,5-a]pyridin-3-yl)piperazine-1-carboxylate (Compound 220)

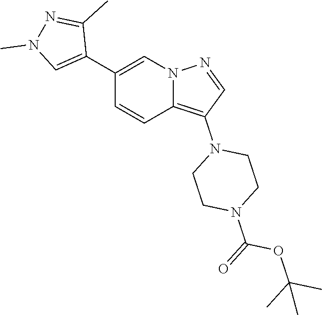 [SMILES: Cc1nn(C)cc1-c1ccc2c(N3CCN(C(=O)OC(C)(C)C)CC3)cnn2c1]

### Step 1. Preparation of 6-(1,3-dimethyl-1H-pyrazol-4-yl)pyrazolo[1,5-a]pyridine

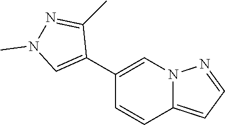 [SMILES: Cc1nn(C)cc1-c1ccc2ccnn2c1]

To a solution of 6-bromopyrazolo[1,5-a]pyridine (1 g, 5.08 mmol) and 1,3-dimethylpyrazol-4-ylboronic acid (0.85 g, 6.09 mmol) in dioxane (20 mL) and H₂O (2 mL) were added K₂CO₃ (1.40 g, 10.15 mmol) and Pd(dppf)Cl₂·DCM (0.74 g, 1.02 mmol). The reaction was stirred overnight at 90° C. under nitrogen. The mixture was allowed to cool to rt then concentrated under reduced pressure. The residue was dissolved in water (100 mL), extracted with EtOAc (3×100 mL) and dried over anhydrous Na₂SO₄. After filtration, the filtrate was concentrated under reduced pressure. The residue was purified by silica gel column chromatography eluting with PE/EtOAc (1:1) to afford 850 mg (79%) of 6-(1,3-dimethyl-1H-pyrazol-4-yl)pyrazolo[1,5-a]pyridine as a yellow oil. ¹H NMR (400 MHZ, DMSO-d₆) δ (ppm) 8.65 (s, 1H), 8.03-7.95 (m, 2H), 7.80-7.70 (d, J=9.1 Hz, 1H), 7.36-7.26 (d, J=9.2 Hz, 1H), 6.61 (s, 1H), 3.80 (s, 3H), 2.33 (s, 3H). MS (ESI) m/z [M+H]⁺ calcd. for C₁₂H₁₂N₄, 213.1; found, 213.1.

### Step 2. Preparation of 3-bromo-6-(1,3-dimethyl-1H-pyrazol-4-yl)pyrazolo[1,5-a]pyridine

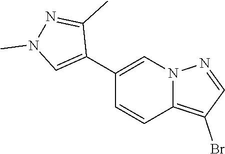 [SMILES: Cc1nn(C)cc1-c1ccc2c(Br)cnn2c1]

To a solution of 6-(1,3-dimethyl-1H-pyrazol-4-yl)pyrazolo[1,5-a]pyridine (800 mg, 3.77 mmol, as prepared in the previous step) in DCM (20 mL) were added NBS (737.9 mg, 4.15 mmol). The reaction was stirred for 2 h at rt under nitrogen. The resulting mixture was washed with water (3×50 mL). The aqueous layer was extracted with DCM (3×100 mL). The resulting mixture was concentrated under reduced pressure. The residue was purified by silica gel column chromatography eluting with PE/EtOAc (3:2) to afford 780 mg (71%) of 3-bromo-6-(1,3-dimethyl-1H-pyrazol-4-yl)pyrazolo[1,5-a]pyridine as a grey solid. ¹H NMR (400 MHz, DMSO-d₆) δ (ppm) 8.71 (s, 1H), 8.15 (s, 1H), 8.04 (s, 1H), 7.61 (d, J=9.1 Hz, 1H), 7.47 (d, J=9.2 Hz, 1H), 3.81 (s, 3H), 2.34 (s, 3H). MS (ESI) m/z [M+H]⁺ calcd. for C₁₂H₁₁BrN₄, 292.1; found, 292.1.

### Step 3. tert-butyl 4-(6-(1,3-dimethyl-1H-pyrazol-4-yl)pyrazolo[1,5-a]pyridin-3-yl)piperazine-1-carboxylate (Compound 220)

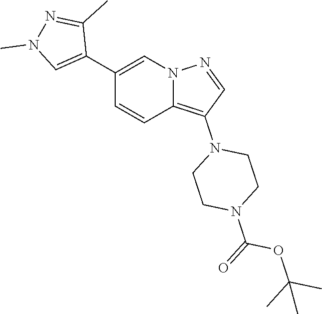 [SMILES: Cc1nn(C)cc1-c1ccc2c(N3CCN(C(=O)OC(C)(C)C)CC3)cnn2c1]

To a solution of 3-bromo-6-(1,3-dimethyl-1H-pyrazol-4-yl)pyrazolo[1,5-a]pyridine (600 mg, 2.06 mmol, as prepared in the previous step) and tert-butyl piperazine-1-carboxylate (1.92 g, 10.31 mmol) in dioxane (10 mL) and tBuOH (10 mL) were added KOtBu (462.5 mg, 4.12 mmol) and tBuXPhos Pd G1 (212.3 mg, 0.31 mmol). The reaction was stirred overnight at 90° C. under nitrogen then allowed to cool to rt. The resulting mixture was concentrated under reduced pressure. The residue was dissolved in water (100 mL). The aqueous layer was extracted with EtOAc (3×100 mL). The combined organic extracts were dried over anhydrous Na₂SO₄ and filtered. The filtrate was concentrated under reduced pressure then the residue was purified by Prep-HPLC to afford 211.3 mg (26%) of tert-butyl 4-(6-(1,3-dimethyl-1H-pyrazol-4-yl)pyrazolo[1,5-a]pyridin-3-yl)piperazine-1-carboxylate (Compound 220) as a yellow solid. ¹H NMR (400 MHZ, DMSO-d₆) δ (ppm) 8.49 (s, 1H), 7.98 (s, 1H), 7.79 (s, 1H), 7.65 (d, J=9.2 Hz, 1H), 7.17 (d, J=9.3 Hz, 1H), 3.79 (s, 3H), 3.51 (t, J=4.9 Hz, 4H), 2.94 (t, J=5.0 Hz, 4H), 2.32 (s, 3H), 1.43 (s, 9H). MS (ESI) m/z [M+H]⁺ calcd. for C₂₁H₂₈N₆O₂, 397.2; found, 397.1; HPLC purity: 254 nm: 99.0%.

### Example 164: Synthesis of Exemplary Compound 221

### tert-butyl 4-(6-(1,5-dimethyl-1H-pyrazol-4-yl)pyrazolo[1,5-a]pyridin-3-yl)piperazine-1-carboxylate (Compound 221)

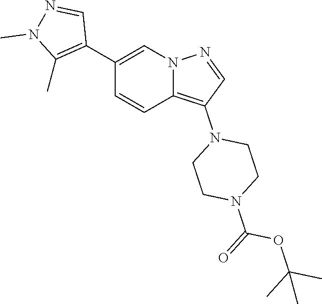 [SMILES: Cc1c(-c2ccc3c(N4CCN(C(=O)OC(C)(C)C)CC4)cnn3c2)cnn1C]

### Step 1. Preparation of 6-(1,5-dimethyl-1H-pyrazol-4-yl)pyrazolo[1,5-a]pyridine

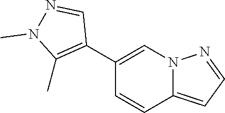 [SMILES: Cc1c(-c2ccc3ccnn3c2)cnn1C]

To a solution of 6-bromopyrazolo[1,5-a]pyridine (1 g, 5.07 mmol) and (1,5-dimethyl-1H-pyrazol-4-yl) boronic acid (852.3 mg, 6.09 mmol) in dioxane (20 mL) and H₂O (0.2 mL) were added K₃PO₄ (2154.6 mg, 10.15 mmol) and Pd(dppf)Cl₂ (742.7 mg, 1.01 mmol). The reaction was for 16 h at 90° C. under nitrogen then concentrated under reduced pressure. The resulting mixture was extracted with EtOAc (3×50 mL). The combined organic layers were washed with brine (10 mL) and dried over anhydrous Na₂SO₄. After filtration, the filtrate was concentrated under reduced pressure. The residue was purified by Prep-TLC (PE/EtOAc 1:1) to afford 950 mg (88%) of 6-(1,5-dimethyl-1H-pyrazol-4-yl)pyrazolo[1,5-a]pyridine as a white solid. ¹H NMR (400 MHZ, DMSO-d₆) δ (ppm) 8.65 (s, 1H), 7.99 (d, J=2.3 Hz, 1H), 7.73 (d, J=9.1 Hz, 1H), 7.67 (s, 1H), 7.30 (dd, J=9.1, 1.6 Hz, 1H), 6.62 (d, J=2.3 Hz, 1H), 3.80 (s, 3H), 2.40 (s, 3H). MS (ESI) m/z [M+H]⁺ calcd. for C₁₂H₁₂N₄, 213.1; found, 213.1.

### Step 2. Preparation of 3-bromo-6-(1,5-dimethyl-1H-pyrazol-4-yl)pyrazolo[1,5-a]pyridine

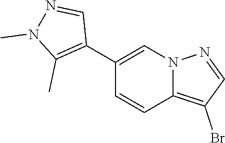 [SMILES: Cc1c(-c2ccc3c(Br)cnn3c2)cnn1C]

To a solution of 6-(1,5-dimethyl-1H-pyrazol-4-yl)pyrazolo[1,5-a]pyridine (900 mg, 4.24 mmol, as prepared in the previous step) in DCM (10 mL) was added NBS (830.2 mg, 4.66 mmol). The reaction was stirred for 1 h at 0° C. under nitrogen then concentrated under reduced pressure. The residue was diluted with water and extracted with DCM (3×50 mL). The combined organic layers were washed with brine (10 mL), dried over anhydrous Na₂SO₄. After filtration, the filtrate was concentrated under reduced pressure. The residue was purified by silica gel column chromatography eluting with DCM/MeOH (5:1) to afford 800 mg (65%) of 3-bromo-6-(1,5-dimethyl-1H-pyrazol-4-yl)pyrazolo[1,5-a]pyridine as a white solid. ¹H NMR (400 MHZ, DMSO-d₆) δ (ppm) 8.73 (s, 1H), 8.16 (s, 1H), 7.70 (s, 1H), 7.62 (d, J=9.1 Hz, 1H), 7.46 (d, J=9.2, 1.5 Hz, 1H), 3.81 (s, 3H), 2.41 (s, 3H). MS (ESI) m/z [M+H]⁺ calcd. for C₁₂H₁₁BrN₄, 290.0; found, 290.1.

### Step 3. tert-butyl 4-(6-(1,5-dimethyl-1H-pyrazol-4-yl)pyrazolo[1,5-a]pyridin-3-yl)piperazine-1-carboxylate (Compound 221)

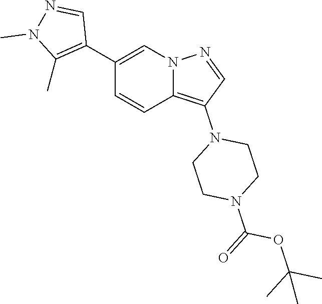 [SMILES: Cc1c(-c2ccc3c(N4CCN(C(=O)OC(C)(C)C)CC4)cnn3c2)cnn1C]

To a solution of 3-bromo-6-(1,5-dimethyl-1H-pyrazol-4-yl)pyrazolo[1,5-a]pyridine (600 mg, 1.63 mmol, as prepared in the previous step) and tert-butyl piperazine-1-carboxylate (1.92 g, 10.31 mmol) in tBuOH (5 mL) and dioxane (5 mL) were added KOtBu (693.7 mg, 6.18 mmol) and tBuXPhos Pd G1 (212.3 mg, 0.31 mmol). The reaction was stirred for 16 h at 90° C. under nitrogen then concentrated under reduced pressure. The residue was diluted with water and extracted with EtOAc (3×50 mL). The combined organic layers were washed with brine (10 mL), dried over anhydrous Na₂SO₄. After filtration, the filtrate was concentrated under reduced pressure. The crude product was purified by Prep-HPLC to afford 108.7 mg (13%) of tert-butyl 4-(6-(1,5-dimethyl-1H-pyrazol-4-yl)pyrazolo[1,5-a]pyridin-3-yl)piperazine-1-carboxylate (Compound 221) as a yellow solid. ¹H NMR (400 MHZ, DMSO-d₆) δ (ppm) 8.48 (s, 1H), 7.80 (s, 1H), 7.68-7.62 (m, 2H), 7.16 (dd, J=9.2, 1.5 Hz, 1H), 3.79 (s, 3H), 3.51 (t, J=5.0 Hz, 4H), 2.94 (t, J=5.0 Hz, 4H), 2.39 (s, 3H), 1.43 (s, 9H). MS (ESI) m/z [M+H]⁺ calcd. for C₂₁H₂₈N₆O₂, 397.2; found, 397.2; HPLC purity: 254 nm: 98.7%.

### Example 165: Synthesis of Exemplary Compound 222

### tert-butyl 4-(6-(1,3,5-trimethyl-1H-pyrazol-4-yl)pyrazolo[1,5-a]pyridin-3-yl)piperazine-1-carboxylate (Compound 222)

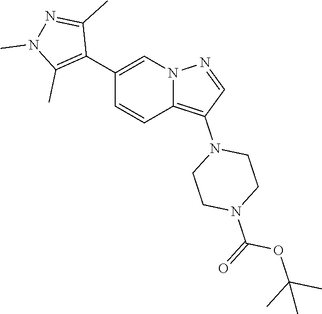 [SMILES: Cc1nn(C)c(C)c1-c1ccc2c(N3CCN(C(=O)OC(C)(C)C)CC3)cnn2c1]

### Step 1. Preparation of 6-(1,3,5-trimethyl-1H-pyrazol-4-yl)pyrazolo[1,5-a]pyridine

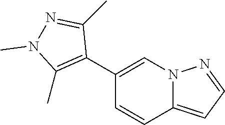 [SMILES: Cc1nn(C)c(C)c1-c1ccc2ccnn2c1]

To a solution of 6-bromopyrazolo[1,5-a]pyridine (1.00 g, 5.08 mmol) and 1,3,5-trimethyl-4-(4,4,5,5-tetramethyl-1,3,2-dioxaborolan-2-yl)pyrazole (1.20 g, 5.08 mmol) in dioxane (10.0 mL) and H₂O (1.0 mL) was added XPhos Pd G3 (0.43 g, 0.51 mmol), XPhos (0.24 g, 0.50 mmol) and K₃PO₄ (2.15 g, 10.1 mmol) under nitrogen. The reaction was stirred for 2 h at 80° C. under nitrogen then concentrated under reduced pressure. The residue was purified by silica gel column chromatography eluting with PE/EtOAc (2:1) to afford 1.0 g (78%) of 6-(1,3,5-trimethyl-1H-pyrazol-4-yl)pyrazolo[1,5-a]pyridine as a yellow oil. ¹H NMR (400 MHZ, DMSO-d₆) δ (ppm) 8.53 (s, 1H), 8.00 (d, J=2.3 Hz, 1H), 7.73 (d, J=9.1 Hz, 1H), 7.15 (dd, J=9.0, 1.6 Hz, 1H), 6.63 (d, J=2.2 Hz, 1H), 3.72 (s, 3H), 2.25 (s, 3H), 2.15 (s, 3H). MS (ESI) m/z [M+H]⁺ calcd. for C₁₃H₁₄N₄, 227.1; found, 227.2.

### Step 2. Preparation of 3-bromo-6-(1,3,5-trimethyl-1H-pyrazol-4-yl)pyrazolo[1,5-a]pyridine

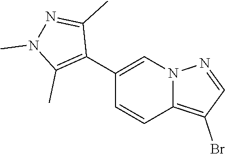 [SMILES: Cc1nn(C)c(C)c1-c1ccc2c(Br)cnn2c1]

To a solution of 6-(1,3,5-trimethyl-1H-pyrazol-4-yl)pyrazolo[1,5-a]pyridine (1.05 g, 4.64 mmol, as prepared in the previous step) in DCM (10.0 mL) was added NBS (0.91 g, 5.10 mmol). The reaction was stirred for 2 h at rt then concentrated under reduced pressure. The residue was purified by Prep-TLC (EtOAc) to afford 1.03 g (55%) of 3-bromo-6-(1,3,5-trimethyl-1H-pyrazol-4-yl)pyrazolo[1,5-a]pyridine as a green oil. ¹H NMR (400 MHZ, DMSO-d₆) δ (ppm) 8.62 (s, 1H), 8.17 (s, 1H), 7.62 (dd, J=9.1, 1.0 Hz, 1H), 7.30 (dd, J=9.1, 1.5 Hz, 1H), 3.72 (s, 3H), 2.25 (s, 3H), 2.16 (s, 3H). MS (ESI) m/z [M+H]⁺ calcd. for C₁₃H₁₃BrN₄, 305.0; found, 305.1.

### Step 3. Preparation of tert-butyl 4-(6-(1,3,5-trimethyl-1H-pyrazol-4-yl)pyrazolo[1,5-a]pyridin-3-yl)piperazine-1-carboxylate (Compound 222)

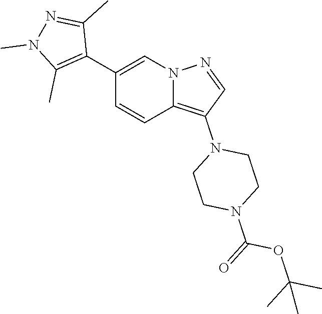 [SMILES: Cc1nn(C)c(C)c1-c1ccc2c(N3CCN(C(=O)OC(C)(C)C)CC3)cnn2c1]

To a solution of 3-bromo-6-(1,3,5-trimethyl-1H-pyrazol-4-yl)pyrazolo[1,5-a]pyridine (790 mg, 2.59 mmol, as prepared in the previous step) and tert-butyl piperazine-1-carboxylate (482 mg, 2.59 mmol) in tBuOH (8.0 mL) and dioxane (4.0 mL) was added KOtBu (436 mg, 3.88 mmol) and tBuXPhos Pd G1 (267 mg, 0.39 mmol) under nitrogen. The reaction was stirred for 16 h at 80° C. under nitrogen then concentrated under reduced pressure. The residue was purified by silica gel column chromatography eluting with PE/EtOAc (1:1) then further purified by Prep-HPLC to afford 60.7 mg (6%) of tert-butyl 4-(6-(1,3,5-trimethyl-1H-pyrazol-4-yl)pyrazolo[1,5-a]pyridin-3-yl)piperazine-1-carboxylate (Compound 222) as a yellow solid. ¹H NMR (400 MHZ, DMSO-d₆) δ (ppm) 8.38 (s, 1H), 7.82 (s, 1H), 7.66 (d, J=9.2 Hz, 1H), 7.01 (d, J=9.2 Hz, 1H), 3.71 (s, 3H), 3.52 (t, J=4.9 Hz, 4H), 2.96 (t, J=5.0 Hz, 4H), 2.24 (s, 3H), 2.14 (s, 3H), 1.44 (s, 9H). MS (ESI) m/z [M+H]⁺ calcd. for C₂₂H₃₀N₆O₂, 411.2; found, 411.25; HPLC purity: 254 nm: 97.9%.

### Example 166: Synthesis of Exemplary Compound 223

### tert-butyl 4-(6-(1-methylpyrrolidin-3-yl)pyrazolo[1,5-a]pyridin-3-yl)piperazine-1-carboxylate (Compound 223)

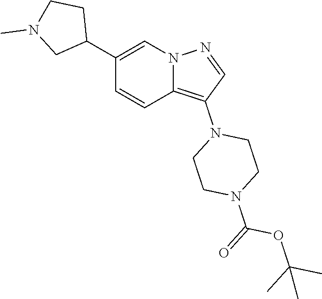 [SMILES: CN1CCC(c2ccc3c(N4CCN(C(=O)OC(C)(C)C)CC4)cnn3c2)C1]

### Step 1. Preparation of 6-(1-methyl-2,5-dihydro-1H-pyrrol-3-yl)pyrazolo[1,5-a]pyridine

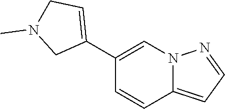

To a stirred solution of 6-bromopyrazolo[1,5-a]pyridine (500 mg, 2.54 mmol) and 1-methyl-3-(4,4,5,5-tetramethyl-1,3,2-dioxaborolan-2-yl)-2,5-dihydro-1H-pyrrole (689.8 mg, 3.30 mmol) in dioxane (15 mL) and H₂O (1.5 mL) were added Pd(dppf)Cl₂·DCM (206.7 mg, 0.25 mmol) and K₂CO₃ (701.4 mg, 5.08 mmol) at rt. The reaction was stirred for 2 h at 90° C. under nitrogen then diluted with water (50 mL). The resulting mixture was extracted with DCM (3×100 mL). The combined organic layers were dried over anhydrous Na₂SO₄. After filtration, the filtrate was concentrated under reduced pressure. The residue was purified by silica gel column chromatography eluting with DCM/MeOH (5:1) to afford 160 mg (32%) of 6-(1-methyl-2,5-dihydro-1H-pyrrol-3-yl)pyrazolo[1,5-a]pyridine as a brown solid. ¹H NMR (400 MHz, DMSO-d₆) δ (ppm) 8.50 (s, 1H), 7.99 (d, J=2.2 Hz, 1H), 7.67 (d, J=9.2 Hz, 1H), 7.49 (dd, J=9.3, 1.6 Hz, 1H), 6.60 (d, J=1.8 Hz, 1H), 6.44-5.37 (m, 1H), 3.80-3.74 (m, 2H), 3.58-3.54 (m, 2H), 2.45 (s, 3H). MS (ESI) m/z [M+H]⁺ calcd. for C₁₂H₁₃N₃, 200.1; found, 200.2.

### Step 2. Preparation of 6-(1-methylpyrrolidin-3-yl)pyrazolo[1,5-a]pyridine

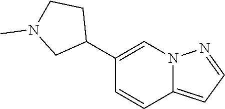

To a stirred solution of 6-(1-methyl-2,5-dihydro-1H-pyrrol-3-yl)pyrazolo[1,5-a]pyridine (130 mg, 0.65 mmol, as prepared in the previous step) in MeOH (5 mL) was added Pd/C (13.9 mg, 0.13 mmol) at rt. The reaction was stirred for 1 h at rt under hydrogen atmosphere. The resulting mixture was filtered, and the filter cake was washed with DCM (3×50 mL). The filtrate was concentrated under reduced pressure. The residue was purified by reversed-phase flash chromatography to afford 50 mg (38%) of 6-(1-methylpyrrolidin-3-yl)pyrazolo[1,5-a]pyridine as a yellow oil. ¹H NMR (400 MHZ, DMSO-d₆) δ (ppm) 8.52 (s, 1H), 7.92 (d, J=2.3 Hz, 1H), 7.65 (d, J=9.1 Hz, 1H), 7.23 (dd, J=9.1, 1.6 Hz, 1H), 6.54 (d, J=2.2 Hz, 1H), 3.43-3.37 (m, 1H), 2.83-2.76 (m, 1H), 2.73-2.65 (m, 1H), 2.60-2.54 (m, 1H), 2.49-2.45 (m, 1H), 2.36-2.19 (m, 4H), 1.85-1.72 (m, 1H). MS (ESI) m/z [M+H]⁺ calcd. for C₁₂H₁₅N₃, 202.1; found, 202.2.

### Step 3. Preparation of 3-bromo-6-(1-methylpyrrolidin-3-yl)pyrazolo[1,5-a]pyridine

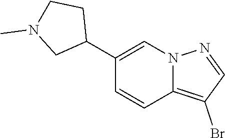 [SMILES: CN1CCC(c2ccc3c(Br)cnn3c2)C1]

To a stirred solution of 6-(1-methylpyrrolidin-3-yl)pyrazolo[1,5-a]pyridine (40 mg, 0.20 mmol, as prepared in the previous step) in DCM (4 mL) was added NBS (35.4 mg, 0.20 mmol) at rt. The reaction was stirred for 2 h at rt. The resulting mixture was filtered, and the filter cake was washed with DCM (3×20 mL). The filtrate was concentrated under reduced pressure. The residue was purified by silica gel column chromatography eluting with DCM/MeOH (10:1) to afford 50 mg (90%) of 3-bromo-6-(1-methylpyrrolidin-3-yl)pyrazolo[1,5-a]pyridine as a yellow oil. ¹H NMR (400 MHZ, DMSO-d₆) δ (ppm) 8.59 (s, 1H), 8.09 (s, 1H), 7.55 (d, J=9.2 Hz, 1H), 7.40 (dd, J=9.2, 1.5 Hz, 1H), 3.53-3.38 (m, 1H), 2.85-2.64 (m, 2H), 2.58-2.52 (m, 2H), 2.36-2.21 (m, 4H), 1.86-1.66 (m, 1H). MS (ESI) m/z [M+H]⁺ calcd. for C₁₂H₁₄BrN₃, 280.0; found, 280.1.

### Step 4. Preparation of tert-butyl 4-(6-(1-methylpyrrolidin-3-yl)pyrazolo[1,5-a]pyridin-3-yl)piperazine-1-carboxylate (Compound 223)

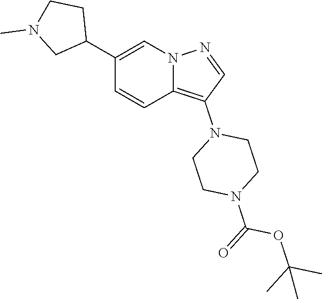 [SMILES: CN1CCC(c2ccc3c(N4CCN(C(=O)OC(C)(C)C)CC4)cnn3c2)C1]

To a stirred solution of 3-bromo-6-(1-methylpyrrolidin-3-yl)pyrazolo[1,5-a]pyridine (40 mg, 0.14 mmol, as prepared in the previous step) and tert-butyl piperazine-1-carboxylate (132.9 mg, 0.72 mmol) in tBuOH (3 mL) and dioxane (3 mL) were added KOtBu (24.1 mg, 0.21 mmol) and tBuXPhos Pd G1 (19.6 mg, 0.03 mmol) at rt. The resulting mixture was stirred for 16 h at 50° C. under nitrogen then concentrated under reduced pressure. The residue was dissolved in water (100 mL) and extracted with DCM (3×80 mL). The combined organic layers were dried over anhydrous Na₂SO₄. After filtration, the filtrate was concentrated under reduced pressure. The residue was purified by silica gel column chromatography eluting with DCM/MeOH (10:1) and then purified by Prep-HPLC to afford 13.6 mg (25%) of tert-butyl 4-(6-(1-methylpyrrolidin-3-yl)pyrazolo[1,5-a]pyridin-3-yl)piperazine-1-carboxylate (Compound 223) as a yellow semi-solid. ¹H NMR (400 MHZ, DMSO-d₆) δ (ppm) 8.37 (s, 1H), 7.73 (s, 1H), 7.58 (d, J=9.2 Hz, 1H), 7.14-7.05 (m, 1H), 3.49 (t, J=5.1 Hz, 4H), 2.91 (t, J=5.1 Hz, 4H), 2.80-2.74 (m, 1H), 2.71-2.63 (m, 1H), 2.59-2.52 (m, 1H), 2.47-2.42 (m, 1H), 2.35-2.16 (m, 5H), 1.86-1.68 (m, 1H), 1.42 (s, 9H). MS (ESI) m/z [M+H]⁺ calcd. for C₂₁H₃₁N₅O₂, 386.2; found, 386.30; HPLC purity: 220 nm: 99.1%.

### Example 167: Synthesis of Exemplary Compound 224

### tert-butyl-4-(6-(tetrahydro-2H-pyran-4-yl)pyrazolo[1,5-a]pyridin-3-yl)piperazine-1-carboxylate (Compound 224)

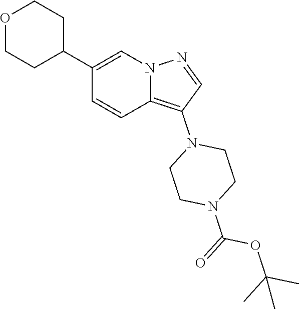 [SMILES: CC(C)(C)OC(=O)N1CCN(c2cnn3cc(C4CCOCC4)ccc23)CC1]

### Step 1. Preparation of 6-(3,6-dihydro-2H-pyran-4-yl)pyrazolo[1,5-a]pyridine

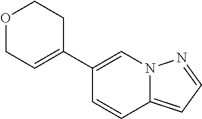 [SMILES: C1=C(c2ccc3ccnn3c2)CCOC1]

To a solution of 6-bromopyrazolo[1,5-a]pyridine (2.00 g, 10.20 mmol) dissolved in dioxane (15 mL) and H₂O (1.5 mL) was added 2-(3,6-dihydro-2H-pyran-4-yl)-4,4,5,5-tetramethyl-1,3,2-dioxaborolane (2.14 g, 10.20 mmol), Pd(dppf)Cl₂·DCM (1.24 g, 1.55 mmol) and K₃PO₄ (6.46 g, 30.40 mmol). The reaction was stirred for 16 h at 90° C. under nitrogen then allowed to cool to rt. The resulting mixture was diluted with water (20 mL) and extracted with EtOAc (3×30 mL). The organic layers were combined and concentrated. The residue was purified by silica gel chromatography with EtOAc/PE (1/1) to afford 2.00 g (94%) of 6-(3,6-dihydro-2H-pyran-4-yl)pyrazolo[1,5-a]pyridine as a green solid. ¹H NMR (400 MHZ, DMSO-d₆) δ (ppm) 8.63 (s, 1H), 7.98 (d, J=2.2 Hz, 1H), 7.67 (d, J=9.3 Hz, 1H), 7.48-7.42 (m, 1H), 6.60-6.59 (m, 1H), 6.40 (s, 1H) 4.27-4.24 (m, 2H) 3.94 (s, 2H) 3.84 (t, J=5.5 Hz, 2H) 1.08 (s, 9H). MS (ESI) m/z [M+H]⁺ calcd. for C₁₂H₁₂N₂O, 201.1; found, 201.2.

### Step 2. Preparation of 6-(tetrahydro-2H-pyran-4-yl)pyrazolo[1,5-a]pyridine

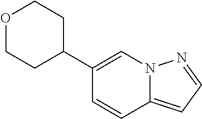 [SMILES: c1cc2ccc(C3CCOCC3)cn2n1]

To a solution of 6-(3,6-dihydro-2H-pyran-4-yl)pyrazolo[1,5-a]pyridine (1.00 g, 4.99 mmol, as prepared in the previous step) dissolved in MeOH (20.0 mL) was added Pd/C (1.00 g, 9.39 mmol). The reaction was stirred at rt for 2 h under hydrogen atmosphere. The resulting mixture was filtered, and the filter cake was washed with MeOH (3×20 mL). The filtrate was concentrated under reduced pressure. The residue was filtered to afford 550 mg (50%) of 6-(tetrahydro-2H-pyran-4-yl)pyrazolo[1,5-a]pyridine as a grey solid. ¹H NMR (400 MHz, DMSO-d₆) δ (ppm) 8.49 (s, 1H), 7.93 (s, 1H), 7.65 (d, J=9.1 Hz, 1H), 7.21 (d, J=9.1 Hz, 1H), 6.55 (d, J=2.3 Hz, 1H), 4.00-3.93 (m, 4H) 3.44 (t, J=8.0 Hz, 2H) 2.87-2.80 (m, 1H) 1.77-1.69 (m, 4H) 1.07 (s, 9H). MS (ESI) m/z [M+H]⁺ calcd. for C₁₂H₁₄N₂O, 203.1; found, 203.2.

### Step 3. Preparation of 3-bromo-6-(tetrahydro-2H-pyran-4-yl)pyrazolo[1,5-a]pyridine

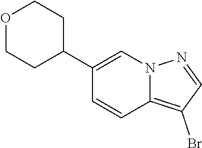 [SMILES: Brc1cnn2cc(C3CCOCC3)ccc12]

To a solution of 6-(tetrahydro-2H-pyran-4-yl)pyrazolo[1,5-a]pyridine (530 mg, 2.62 mmol, as prepared in the previous step) dissolved in DCM (15 mL) was added NBS (513 mg, 2.88 mmol). The reaction was stirred for 2 h at rt, the concentrated under reduced pressure. The residue was purified by a silica gel column with EtOAc/PE (1/1) to afford 380 mg (45%) of 3-bromo-6-(tetrahydro-2H-pyran-4-yl)pyrazolo[1,5-a]pyridine as a red solid. ¹H NMR (400 MHz, DMSO-d₆) δ (ppm) 8.58 (s, 1H), 8.11 (s, 1H), 7.54 (d, J=9.1 Hz, 1H), 7.38 (d, J=9.1 Hz, 1H), 3.97-3.95 (m, 2H), 3.48-3.40 (m, 2H), 2.91-2.84 (m, 1H), 1.80-1.70 (m, 4H). MS (ESI) m/z [M+H]⁺ calcd. for C₁₂H₁₃BrN₂O, 281.0; found, 281.2.

### Step 4. Preparation of tert-butyl-4-(6-(tetrahydro-2H-pyran-4-yl)pyrazolo[1,5-a]pyridin-3-yl)piperazine-1-carboxylate (Compound 224)

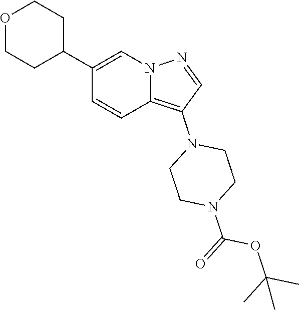 [SMILES: CC(C)(C)OC(=O)N1CCN(c2cnn3cc(C4CCOCC4)ccc23)CC1]

To a solution of 3-bromo-6-(tetrahydro-2H-pyran-4-yl)pyrazolo[1,5-a]pyridine (600 mg, 2.62 mmol, as prepared in the previous step) dissolved in tBuOH (5.00 mL) and dioxane (5 mL) was added tert-butyl piperazine-1-carboxylate (1.26 g, 6.76 mmol), KOtBu (228 mg, 2.03 mmol) and tBuXPhos Pd G1 (139 mg, 0.20 mmol). The reaction was stirred for 16 h at 90° C. under nitrogen then allowed to cool to rt. The resulting mixture was diluted with water (15.0 mL) and extracted with EtOAc (3×30 mL). The organic layers were combined and concentrated. The residue was applied onto a silica gel column and eluting with EtOAc/PE (1/1). The crude product was further purified by Prep-HPLC to afford 31.9 mg (6%) of tert-butyl 4-[6-(oxan-4-yl)pyrazolo[1,5-a]pyridin-3-yl]piperazine-1-carboxylate (Compound 224) as a white solid. ¹H NMR (400 MHZ, DMSO-d₆) δ (ppm) 8.34 (s, 1H), 7.75 (d, J=0.8 Hz, 1H), 7.58 (d, J=9.2 Hz, 1H), 7.08 (d, J=9.3 Hz, 1H), 3.96 (d, J=10.7 Hz, 2H), 3.50 (t, J=5.0 Hz, 4H), 3.44 (d, J=4.0 Hz, 2H), 2.91 (t, J=5.0 Hz, 4H), 2.82-2.75 (m, 1H), 1.77-1.69 (m, 4H), 1.43 (s, 9H). MS (ESI) m/z [M+H]⁺ calcd. for C₂₁H₃₀N₄O₃, 387.2; found, 387.1; HPLC purity: 220 nm: 96.5%.

### Example 168: Synthesis of Exemplary Compound 225

### tert-butyl 4-(6-(1-methylpiperidin-4-yl)pyrazolo[1,5-a]pyridin-3-yl)piperazine-1-carboxylate (Compound 225)

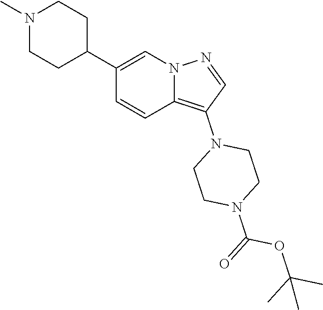 [SMILES: CN1CCC(c2ccc3c(N4CCN(C(=O)OC(C)(C)C)CC4)cnn3c2)CC1]

### Step 1. Preparation of 6-(1-methyl-1,2,3,6-tetrahydropyridin-4-yl)pyrazolo[1,5-a]pyridine

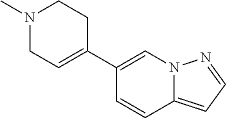 [SMILES: CN1CC=C(c2ccc3ccnn3c2)CC1]

To a solution of 6-bromopyrazolo[1,5-a]pyridine (2 g, 10.15 mmol) and 1-methyl-4-(4,4,5,5-tetramethyl-1,3,2-dioxaborolan-2-yl)-3,6-dihydro-2H-pyridine (3.4 g, 15.23 mmol) in dioxane (15 mL) and H₂O (1.5 mL) was added Pd(dppf)Cl₂ (1.1 g, 1.52 mmol) and K₃PO₄ (6.4 g, 30.45 mmol) at rt. The reaction was stirred for 16 h at 90° C. then was diluted with water (200 mL). The resulting mixture was extracted with EtOAc (3×200 mL). The combined organic layers were dried over anhydrous Na₂SO₄. After filtration, the filtrate was concentrated under reduced pressure. The residue was purified by silica gel column chromatography eluting with PE/EtOAc (1:8) to afford 1.8 g (83%) of 6-(1-methyl-1,2,3,6-tetrahydropyridin-4-yl)pyrazolo[1,5-a]pyridine as a yellow solid. ¹H NMR (400 MHZ, DMSO-d₆) δ (ppm) 8.59 (s, 1H), 7.97 (d, J=2.3 Hz, 1H), 7.64 (d, J=9.3 Hz, 1H), 7.45-7.40 (m, 1H), 6.58 (d, J=2.2 Hz, 1H), 6.30 (t, J=3.6 Hz, 1H), 3.03-2.99 (m, 2H), 2.58 (t, J=6.0 Hz, 2H), 2.54-2.48 (m, 2H), 2.28 (s, 3H). MS (ESI) m/z [M+H]⁺ calcd. for C₁₃H₁₅N₃, 214.1; found, 214.2.

### Step 2 Preparation of 6-(1-methylpiperidin-4-yl)pyrazolo[1,5-a]pyridine

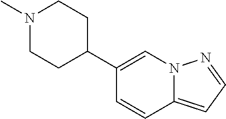 [SMILES: CN1CCC(c2ccc3ccnn3c2)CC1]

To a solution of 6-(1-methyl-1,2,3,6-tetrahydropyridin-4-yl)pyrazolo[1,5-a]pyridine (1.7 g, 7.97 mmol, as prepared in the previous step) in MeOH (15 mL) was added Pd/C (848 mg, 7.97 mmol). The reaction was stirred for 2 h at rt under hydrogen atmosphere. The resulting mixture was filtered, then filter cake was washed with MeOH (3×100 mL). The filtrate was concentrated under reduced pressure to afford 1.1 g (64%) of 6-(1-methylpiperidin-4-yl)pyrazolo[1,5-a]pyridine as a yellow solid. ¹H NMR (400 MHZ, DMSO-d₆) δ (ppm) 8.48 (s, 1H), 7.92 (s, 1H), 7.63 (d, J=9.1 Hz, 1H), 7.18-7.16 (m, 1H), 6.54 (d, J=2.3 Hz, 1H), 3.17 (s, 1H), 2.92-2.75 (m, 4H), 2.16 (m, 3H), 1.97-1.94 (m, 2H), 1.84-1.59 (m, 2H). MS (ESI) m/z [M+H]⁺ calcd. for C₁₃H₁₇N₃, 216.1; found, 216.2.

### Step 3. Preparation of 3-bromo-6-(1-methylpiperidin-4-yl)pyrazolo[1,5-a]pyridine

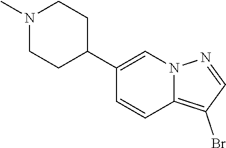 [SMILES: CN1CCC(c2ccc3c(Br)cnn3c2)CC1]

To a solution of 6-(1-methylpiperidin-4-yl)pyrazolo[1,5-a]pyridine (1 g, 4.65 mmol, as prepared in the previous step) in DCM (10 mL) was added NBS (909 mg, 5.11 mmol). The reaction was stirred for 2 h at rt then concentrated under reduced pressure. The crude product was purified by reverse phase flash chromatography to afford 460 mg (67%) of 3-bromo-6-(1-methylpiperidin-4-yl)pyrazolo[1,5-a]pyridine as a yellow solid. ¹H NMR (400 MHz, DMSO-d₆) δ (ppm) 8.56 (s, 1H), 8.10 (s, 1H), 7.56-7.49 (m, 1H), 7.35-7.32 (m, 1H), 2.89-2.86 (m, 2H), 2.57-2.53 (m, 1H), 2.21 (s, 3H), 1.98-1.96 (m, 2H), 1.84-1.63 (m, 4H). MS (ESI) m/z [M+H]⁺ calcd. for C₁₃H₁₆BrN₃, 294.1; found, 294.2.

### Step 4. tert-butyl 4-(6-(1-methylpiperidin-4-yl)pyrazolo[1,5-a]pyridin-3-yl)piperazine-1-carboxylate (Compound 225)

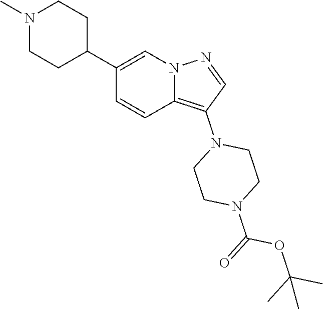 [SMILES: CN1CCC(c2ccc3c(N4CCN(C(=O)OC(C)(C)C)CC4)cnn3c2)CC1]

To a solution of 3-bromo-6-(1-methylpiperidin-4-yl)pyrazolo[1,5-a]pyridine (450 mg, 1.530 mmol, as prepared in the previous step) and tert-butyl piperazine-1-carboxylate (1.4 g, 7.650 mmol) in dioxane (5 mL) and tBuOH (10 mL) was added tBuXPhos Pd G3 (121 mg, 0.15 mmol) and KOtBu (343 mg, 3.06 mmol). The reaction was stirred for 18 h at 80° C. then diluted with H₂O (200 mL). The resulting mixture was extracted with EtOAc (3×200 mL). The combined organic layers were dried over anhydrous Na₂SO₄. After filtration, the filtrate was concentrated under reduced pressure. The crude product was purified by reverse phase flash chromatography to afford crude product. The crude product was purified by Prep-HPLC to afford 47.8 mg (8%) of tert-butyl 4-(6-(1-methylpiperidin-4-yl)pyrazolo[1,5-a]pyridin-3-yl)piperazine-1-carboxylate (Compound 225) as a white solid. ¹H NMR (400 MHZ, DMSO-d₆) δ (ppm) 8.56 (s, 1H), 7.76 (s, 1H), 7.56 (d, J=2.3 Hz, 1H), 7.05 (d, J=2.3 Hz, 1H), 3.53-3.46 (m, 4H), 2.94-2.83 (m, 6H), 2.58-2.50 (m, 1H), 2.20 (d, J=5.8 Hz, 3H), 2.01-1.90 (m, 2H), 1.77-1.69 (m, 4H), 1.42 (s, 9H). MS (ESI) m/z [M+H]⁺ calcd. for C₂₂H₃₃N₅O₂, 400.1; found, 400.2; HPLC purity: 220 nm: 99.1%.

### Example 169: Synthesis of Exemplary Compound 226

### tert-butyl 4-(6-(1-methyl-1H-imidazol-4-yl)pyrazolo[1,5-a]pyridin-3-yl)piperazine-1-carboxylate (Compound 226)

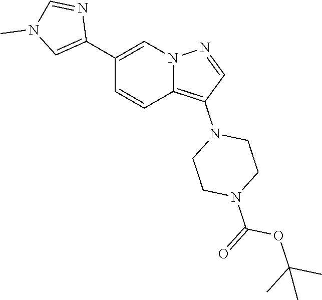 [SMILES: Cn1cnc(-c2ccc3c(N4CCN(C(=O)OC(C)(C)C)CC4)cnn3c2)c1]

### Step 1. Preparation of 6-(1-methyl-1H-imidazol-4-yl)pyrazolo[1,5-a]pyridine

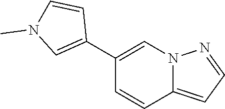

To a solution of 6-bromopyrazolo[1,5-a]pyridine (400 mg, 2.03 mmol) and 1-methyl-4-(4,4,5,5-tetramethyl-1,3,2-dioxaborolan-2-yl)-1H-imidazole (506.8 mg, 2.44 mmol) in dioxane (10 mL) and H₂O (0.1 mL) were added K₃PO₄ (861.8 mg, 4.06 mmol), XPhos Pd G3 (343.68 mg, 0.40 mmol) and XPhos (193.5 mg, 0.40 mmol). The reaction stirred for 16 h at 90° C. under nitrogen then concentrated under reduced pressure. The resulting mixture was extracted with EtOAc (3×50 mL). The combined organic layers were washed with brine (10 mL) and dried over anhydrous Na₂SO₄. After filtration, the filtrate was concentrated under reduced pressure. The residue was purified by Prep-TLC DCM/MeOH (10:1) to afford 220 mg (55%) of as a light-yellow solid. ¹H NMR (400 MHZ, DMSO-d₆) δ (ppm) 8.91 (s, 1H), 7.95 (s, 1H), 7.74-7.66 (m, 2H), 7.60 (dd, J=9.2, 1.5 Hz, 1H), 6.58 (d, J=2.2 Hz, 1H), 3.70 (s, 3H). MS (ESI) m/z [M+H]⁺ calcd. for C₁₁H₁₀N₄, 199.1; found, 199.0.

### Step 2. Preparation of 6-(1H-imidazol-4-yl)-3-iodopyrazolo[1,5-a]pyridine

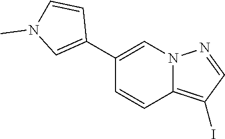 [SMILES: Cn1ccc(-c2ccc3c(I)cnn3c2)c1]

To a solution of 6-(1-methyl-1H-imidazol-4-yl)pyrazolo[1,5-a]pyridine (220 mg, 1.11 mmol, as prepared in the previous step) in DCM (5 mL) were added NIS (374.5 mg, 1.67 mmol). The reaction was stirred for 16 h at rt under nitrogen then concentrated under reduced pressure. The residue was diluted with water and extracted with EtOAc (3×50 mL). The combined organic layers were washed with brine (10 mL) and dried over anhydrous Na₂SO₄. After filtration, the filtrate was concentrated under reduced pressure. The residue was purified by Prep-TLC (DCM/MeOH 10:1) to afford 180 mg (52%) of 6-(1H-imidazol-4-yl)-3-iodopyrazolo[1,5-a]pyridine as a yellow solid. ¹H NMR (400 MHZ, DMSO-d₆) δ (ppm) 8.94 (s, 1H), 8.23-8.04 (m, 1H), 7.92-7.65 (m, 3H), 7.65-7.46 (m, 1H), 3.69 (m, 3H). MS (ESI) m/z [M+H]⁺ calcd. for C₁₀H₇IN₄, 311.1; found, 311.2.

### Step 3. tert-butyl 4-(6-(1-methyl-1H-imidazol-4-yl)pyrazolo[1,5-a]pyridin-3-yl)piperazine-1-carboxylate (Compound 226)

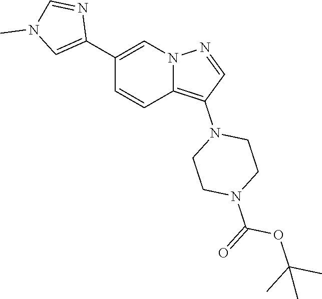 [SMILES: Cn1cnc(-c2ccc3c(N4CCN(C(=O)OC(C)(C)C)CC4)cnn3c2)c1]

To a solution of 6-(1H-imidazol-4-yl)-3-iodopyrazolo[1,5-a]pyridine (160 mg, 0.49 mmol, as prepared in the previous step) and tert-butyl piperazine-1-carboxylate (459.7 mg, 2.47 mmol) in dioxane (5 mL) and tBuOH (5 mL) were added KOtBu (110.8 mg, 0.99 mmol) and tBuXPhos Pd G1 (50.8 mg, 0.07 mmol). The reaction was stirred for 48 h at 60° C. under a nitrogen then concentrated under reduced pressure. The residue was purified by silica gel column chromatography eluting with PE/EtOAc (1:1) to afford the crude product. The crude product was purified by Prep-HPLC to afford 7.8 mg (4%) of tert-butyl 4-(6-(1-methyl-1H-imidazol-4-yl)pyrazolo[1,5-a]pyridin-3-yl)piperazine-1-carboxylate (Compound 226) as a yellow semi-solid. ¹H NMR (400 MHZ, DMSO-d₆) δ (ppm) 8.79 (s, 1H), 7.80 (s, 1H), 7.73 (d, J=1.3 Hz, 1H), 7.71 (d, J=1.3 Hz, 1H), 7.67 (d, J=9.2 Hz, 1H), 7.50 (dd, J=9.3, 1.5 Hz, 1H), 3.73 (s, 3H), 3.54 (t, J=5.0 Hz, 4H), 2.97 (t, J=5.0 Hz, 4H), 1.46 (s, 9H). MS (ESI) m/z [M+H]⁺ calcd. for C₂₀H₂₆N₆O₂, 383.2; found, 383.1; HPLC purity: 254 nm: 96.5%.

### Example 170: Synthesis of Exemplary Compound 227

### tert-butyl 4-(6-(1-(2-methoxyethyl)-1H-imidazol-4-yl)pyrazolo[1,5-a]pyridin-3-yl)piperazine-1-carboxylate (Compound 227)

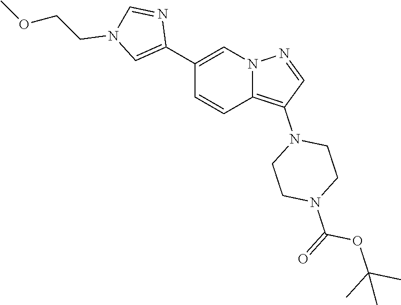 [SMILES: COCCn1cnc(-c2ccc3c(N4CCN(C(=O)OC(C)(C)C)CC4)cnn3c2)c1]

### Step 1. Preparation of 6-(1-(2-methoxyethyl)-1H-imidazol-4-yl)pyrazolo[1,5-a]pyridine

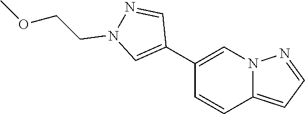 [SMILES: COCCn1cc(-c2ccc3ccnn3c2)cn1]

To a solution of 4-iodo-1-(2-methoxyethyl)-1H-imidazole (Compound S91) (500 mg, 1.98 mmol) and 6-(4,4,5,5-tetramethyl-1,3,2-dioxaborolan-2-yl)pyrazolo[1,5-a]pyridine (581.1 mg, 2.38 mmol) in dioxane (10 mL) and H₂O (1 mL) were added K₃PO₄ (842.1 mg, 3.97 mmol) and Pd(dppf)Cl₂ (290.3 mg, 0.40 mmol). The reaction was stirred for 16 h at 90° C. under nitrogen then concentrated under reduced pressure. The residue was diluted with water and extracted with EtOAc (3×50 mL). The combined organic layers were washed with brine (20 mL), dried over anhydrous Na₂SO₄. After filtration, the filtrate was concentrated under reduced pressure. The residue was purified by silica gel column chromatography eluting with DCM/MeOH (10:1) to afford 280 mg (58%) of 6-(1-(2-methoxyethyl)-1H-imidazol-4-yl)pyrazolo[1,5-a]pyridine as a yellow solid. ¹H NMR (400 MHZ, DMSO-d₆) δ (ppm) 8.92 (s, 1H), 7.97 (s, 1H), 7.89-7.42 (m, 4H), 6.59 (s, 1H), 4.32-4.04 (m, 2H), 3.76-3.56 (m, 2H). 3.28 (s, 3H). MS (ESI) m/z [M+H]⁺ calcd. for C₁₃H₁₄N₄O, 243.1; found, 243.3.

### Step 2. Preparation of 3-iodo-6-(1-(2-methoxyethyl)-1H-imidazol-4-yl)pyrazolo[1,5-a]pyridine

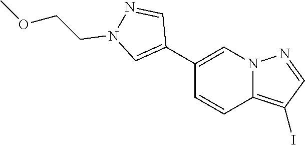 [SMILES: COCCn1cc(-c2ccc3c(I)cnn3c2)cn1]

To a solution of 6-(1-(2-methoxyethyl)-1H-imidazol-4-yl)pyrazolo[1,5-a]pyridine (240 mg, 0.99 mmol, as prepared in the previous step) in DCM (10 mL) was added NIS (222.8 mg, 0.99 mmol). The reaction was stirred for 0.5 h at 0° C. under nitrogen then concentrated under reduced pressure. The residue was diluted with water and extracted with DCM (3×50 mL). The combined organic layers were washed with brine (10 mL) and dried over anhydrous Na₂SO₄. After filtration, the filtrate was concentrated under reduced pressure. The residue was purified by silica gel column chromatography eluting with PE/EtOAc (1:2) to afford 200 mg (55%) of 3-iodo-6-(1-(2-methoxyethyl)-1H-imidazol-4-yl)pyrazolo[1,5-a]pyridine as a yellow solid. ¹H NMR (400 MHZ, DMSO-d₆) δ (ppm) 8.96 (s, 1H), 8.08 (s, 1H), 7.85-7.70 (m, 3H), 7.53 (d, J=9.2 Hz, 1H), 4.18 (t, J=5.2 Hz, 2H), 3.65 (t, J=5.1 Hz, 2H), 3.28 (s, 3H). MS (ESI) m/z [M+H]⁺ calcd. for C₁₃H13I, 369.0; found, 369.1.

### Step 3. tert-butyl 4-(6-(1-(2-methoxyethyl)-1H-imidazol-4-yl)pyrazolo[1,5-a]pyridin-3-yl)piperazine-1-carboxylate (Compound 227)

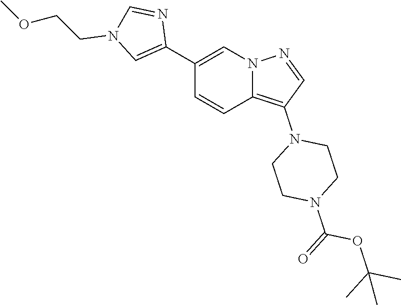 [SMILES: COCCn1cnc(-c2ccc3c(N4CCN(C(=O)OC(C)(C)C)CC4)cnn3c2)c1]

To a solution of 3-iodo-6-(1-(2-methoxyethyl)-1H-imidazol-4-yl)pyrazolo[1,5-a]pyridine (180 mg, 0.49 mmol, as prepared in the previous step) and tert-butyl piperazine-1-carboxylate (455.3 mg, 2.45 mmol) in dioxane (5 mL) and tBuOH (5 mL) were added KOtBu (109.7 mg, 0.98 mmol) and tBuXPhos Pd G1 (50.4 mg, 0.07 mmol). The reaction was stirred for 2 days at 60° C. under nitrogen then concentrated under reduced pressure. The residue was diluted with water and extracted with EtOAc (3×50 mL). The combined organic layers were washed with brine (10 mL), dried over anhydrous Na₂SO₄. After filtration, the filtrate was concentrated under reduced pressure. The crude product was purified by Prep-HPLC to afford 29.2 mg (14%) of tert-butyl 4-(6-(1-(2-(dimethylamino)ethyl)-1H-imidazol-4-yl)pyrazolo[1,5-a]pyridin-3-yl)piperazine-1-carboxylate (Compound 227) as a yellow solid. ¹H NMR (400 MHz, DMSO-d₆) δ (ppm) 8.76 (s, 1H), 7.76 (s, 1H), 7.74-7.69 (m, 2H), 7.65 (d, J=9.3 Hz, 1H), 7.47 (dd, J=9.3, 1.4 Hz, 1H), 4.16 (t, J=5.1 Hz, 2H), 3.65 (t, J=5.1 Hz, 2H), 3.51 (t, J=5.0 Hz, 4H), 3.27 (s, 3H), 2.95 (t, J=5.0 Hz, 4H), 1.43 (s, 9H). MS (ESI) m/z [M+H]⁺ calcd. for C₂₂H₃₀N₆O₃, 427.2; found, 427.2; HPLC purity: 254 nm: 99.5%.

Using the procedures described in Example 170, Compound 227 and reagents, starting materials, and conditions known to those skilled in the art were used to prepare the compounds shown in Table AA.

TABLE AA

| Compound | Name | Characterization |
|---|---|---|
| Compound | tert-butyl 4-(6-(1-(2-methoxyethyl)-1H- | ¹H NMR (400 MHz, DMSO- |
| 228 | imidazol-5-yl)pyrazolo[1,5-a]pyridin-3- | d₆) δ (ppm) 8.65 (s, 1H), 7.88 |
| | yl)piperazine-1-carboxylate | (s, 1H), 7.80 (d, J = 1.3 Hz, |
| | | 1H), 7.71 (d, J = 9.2 Hz, 1H), |
| | | 7.16 (d, J = 9.3 Hz, 1H), 7.07 |
| | | (d, J = 1.2 Hz, 1H), 4.21 (t, J = |
| | | 5.2 Hz, 2H), 3.62-3.51 (m, |
| | | 6H), 3.19 (s, 3H), 2.96 (t, J = |
| | | 5.0 Hz, 4H), 1.43 (s, 9H). MS |
| | | (ESI) m/z [M + H]+ calcd. for |
| | | C₂₂H₃₀N₆O₃, 427.2; |
| | | found, 427.4; HPLC purity: |
| | | 254 nm: 98.9%. |

### Example 171: Synthesis of Exemplary Compound 229

### tert-butyl 4-(6-(1-(2-hydroxyethyl)-1H-imidazol-4-yl)pyrazolo[1,5-a]pyridin-3-yl)piperazine-1-carboxylate (Compound 229)

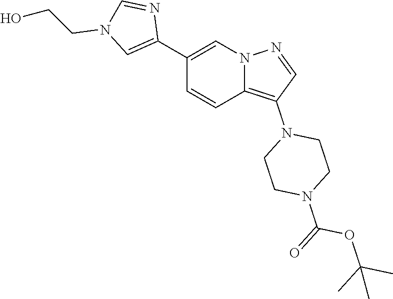 [SMILES: CC(C)(C)OC(=O)N1CCN(c2cnn3cc(-c4cn(CCO)cn4)ccc23)CC1]

### Step 1. Preparation of 6-(1-(2-((tert-butyldimethylsilyl)oxy) ethyl)-1H-imidazol-4-yl)pyrazolo[1,5-a]pyridine

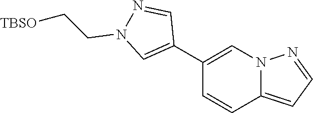 [SMILES: CC(C)(C)[Si](C)(C)OCCn1cc(-c2ccc3ccnn3c2)cn1]

To a solution of 1-{2-[(tert-butyldimethylsilyl)oxy]ethyl}-4-iodoimidazole (Compound S93) (2.5 g, 7.10 mmol) and 6-(4,4,5,5-tetramethyl-1,3,2-dioxaborolan-2-yl)pyrazolo[1,5-a]pyridine (1.73 g, 7.10 mmol) in dioxane (40 mL) and H₂O (4 mL) were added Pd(dppf)Cl₂ (1.04 g, 1.42 mmol) and K₂CO₃ (1.96 g, 14.19 mmol). The reaction was stirred overnight at 90° C. under nitrogen then allowed to cool to rt. The resulting mixture was concentrated under reduced pressure. The residue was dissolved in H₂O (100 mL) and extracted with DCM (3×100 mL). The residue was purified by silica gel column chromatography eluting with PE/EtOAc (5:1) to afford 980 mg (40%) of 6-(1-(2-((tert-butyldimethylsilyl)oxy) ethyl)-1H-imidazol-4-yl)pyrazolo[1,5-a]pyridine as a black oil. MS (ESI) m/z [M+H]⁺ calcd. for C₁₈H₂₆N₄OSi, 343.1; found, 343.1.

### Step 3. Preparation of 2-(4-(pyrazolo[1,5-a]pyridin-6-yl)-1H-imidazol-1-yl)ethan-1-ol

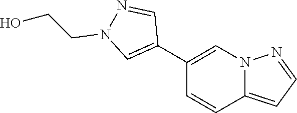 [SMILES: OCCn1cc(-c2ccc3ccnn3c2)cn1]

To a solution of 1-{2-[(tert-butyldimethylsilyl)oxy]ethyl}-4-{pyrazolo[1,5-a]pyridin-6-yl}imidazole (300 mg, 0.88 mmol, as prepared in the previous step) in H₂O (20 mL) was added formic acid (1 mL, 0.04 mmol). The reaction was stirred for 30 min at rt under nitrogen then concentrated under reduced pressure to afford 150 mg (75%) of 2-(4-{pyrazolo[1,5-a]pyridin-6-yl}imidazol-1-yl)ethanol as a white solid. ¹H NMR (400 MHZ, DMSO-d₆) δ (ppm) 8.91 (s, 1H), 7.95 (s, 1H), 7.76 (d, J=1.3 Hz, 1H), 7.74-7.68 (m, 2H), 7.61 (d, J=9.2 Hz, 1H), 6.59 (d, J=2.2 Hz, 1H), 5.05 (s, 1H), 4.05 (t, J=5.4 Hz, 2H), 3.71 (t, J=5.5 Hz, 2H). MS (ESI) m/z [M+H]⁺ calcd. for C₁₂H₁₂N₄O, 229.2; found, 229.3.

### Step 4. Preparation of 2-(4-(3-iodopyrazolo[1,5-a]pyridin-6-yl)-1H-imidazol-1-yl)ethan-1-ol

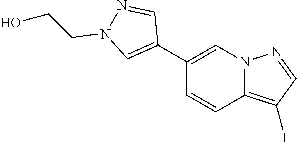 [SMILES: OCCn1cc(-c2ccc3c(I)cnn3c2)cn1]

To a solution of 2-(4-{pyrazolo[1,5-a]pyridin-6-yl}imidazol-1-yl)ethanol (150 mg, 0.66 mmol, as prepared in the previous step) in DCM (15 mL) was added NIS (118.3 mg, 0.53 mmol). The reaction was stirred for 1 h at rt then concentrated under reduced pressure. The residue was dissolved in water (30 mL) and extracted with DCM (3×30 mL) and dried over anhydrous Na₂SO₄. After filtration, the filtrate was concentrated under reduced pressure. The residue was purified by Prep-TLC (DCM/MeOH 10:1) to afford 115.5 mg (62%) of 2-(4-(3-iodopyrazolo[1,5-a]pyridin-6-yl)-1H-imidazol-1-yl)ethan-1-ol as a white solid. ¹H NMR (400 MHz, DMSO-d₆) δ (ppm) 8.98 (s, 1H), 8.10 (s, 1H), 7.87-7.72 (m, 3H), 7.55 (d, J=9.3 Hz, 1H), 5.06 (t, J=5.2 Hz, 1H), 4.07 (t, J=5.4 Hz, 2H), 3.79-3.61 (m, 2H). MS (ESI) m/z [M+H]⁺ calcd. for C₁₂H₁₁IN₄O, 355.1; found, 355.1.

### Step 5. tert-butyl 4-(6-(1-(2-hydroxyethyl)-1H-imidazol-5-yl)pyrazolo[1,5-a]pyridin-3-yl)piperazine-1-carboxylate (Compound 229)

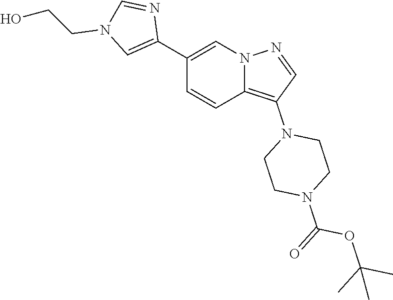 [SMILES: CC(C)(C)OC(=O)N1CCN(c2cnn3cc(-c4cn(CCO)cn4)ccc23)CC1]

To a solution of 2-(4-(3-iodopyrazolo[1,5-a]pyridin-6-yl)-1H-imidazol-1-yl)ethan-1-ol (100 mg, 0.28 mmol, as prepared in the previous step) and tert-butyl piperazine-1-carboxylate (263 mg, 1.41 mmol) in tBuOH (10 mL) and dioxane (10 mL) were added KOtBu (63.4 mg, 0.56 mmol) and tBuXPhos Pd G1 (29.1 mg, 0.04 mmol). The reaction was stirred overnight at 60° C. under nitrogen then allowed to cool to rt. The resulting mixture was concentrated under reduced pressure. The crude product (100 mg) was purified by Prep-HPLC to afford 25 mg (21%) of tert-butyl 4-{6-[1-(2-hydroxyethyl)imidazol-4-yl]pyrazolo[1,5-a]pyridin-3-yl}piperazine-1-carboxylate (Compound 229) as a white solid. ¹H NMR (400 MHz, DMSO-d₆) δ (ppm) 8.76 (s, 1H), 7.79-7.73 (m, 2H), 7.70 (d, J=1.3 Hz, 1H), 7.64 (d, J=9.3 Hz, 1H), 7.48 (d, J=9.3 Hz, 1H), 5.01 (s, 1H), 4.04 (t, J=5.4 Hz, 2H), 3.76-3.65 (m, 2H), 3.51 (t, J=5.0 Hz, 4H), 2.95 (t, J=5.0 Hz, 4H), 1.44 (s, 9H). MS (ESI) m/z [M+H]⁺ calcd. for C₂₁H₂₈N₆O₃, 413.2; found, 413.1; HPLC purity: 254 nm: 96.8%.

Using the procedures described in Example 171, Compound 229 and reagents, starting materials, and conditions known to those skilled in the art were used to prepare the compounds shown in Table BB.

TABLE BB

| Compound | Name | Characterization |
|---|---|---|
| Compound | tert-butyl 4-(6-(1-(2-hydroxyethyl)-1H- | ¹H NMR (400 MHz, DMSO- |
| 230 | imidazol-5-yl)pyrazolo[1,5-a]pyridin-3- | d₆) δ (ppm) 8.69 (s, 1H), 7.88 |
| | yl)piperazine-1-carboxylate | (s, 1H), 7.81 (s, 1H), 7.71 (d, J = |
| | | 9.3 Hz, 1H), 7.18 (d, J = 9.2 |
| | | Hz, 1H), 7.08 (s, 1H), 5.04 (t, |
| | | J = 5.1 Hz, 1H), 4.07 (t, J = 5.6 |
| | | Hz, 2H), 3.68-3.60 (m, 2H), |
| | | 3.52 (t, J = 4.6 Hz, 4H), 2.97 |
| | | (t, J = 5.0 Hz, 4H), 1.44 (s, |
| | | 9H). MS (ESI) m/z [M + H]⁺ |
| | | calcd. for C₂₁H₂₈N₆O₃, 413.4; |
| | | found, 413.2; HPLC purity: |
| | | 254 nm: 99.1%. |

### Example 172: Synthesis of Exemplary Compound 231

### tert-butyl 4-(6-(1-(3-morpholinopropyl)-1H-imidazol-4-yl)pyrazolo[1,5-a]pyridin-3-yl)piperazine-1-carboxylate (Compound 231)

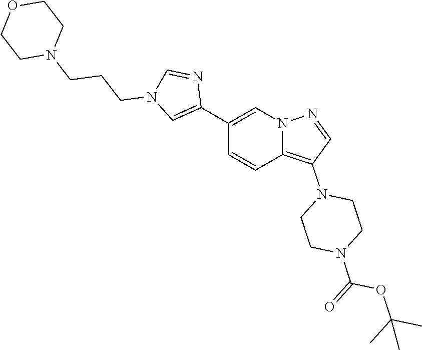 [SMILES: CC(C)(C)OC(=O)N1CCN(c2cnn3cc(-c4cn(CCCN5CCOCC5)cn4)ccc23)CC1]

### Step 1. Preparation of 4-(3-(4-(pyrazolo[1,5-a]pyridin-6-yl)-1H-imidazol-1-yl)propyl)morpholine

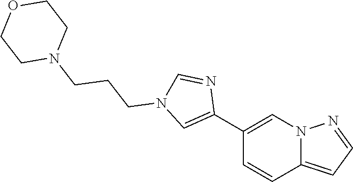 [SMILES: c1cc2ccc(-c3cn(CCCN4CCOCC4)cn3)cn2n1]

To a solution of 4-(3-(4-iodo-1H-imidazol-1-yl)propyl)morpholine (Compound S89) (2.7 g, 8.41 mmol) and 6-(4,4,5,5-tetramethyl-1,3,2-dioxaborolan-2-yl)pyrazolo[1,5-a]pyridine (2.46 g, 10.09 mmol) in dioxane (20 mL) and H₂O (2 mL) was added Pd(dppf)Cl₂ (1.23 g, 1.68 mmol) and K₂CO₃ (2.32 g, 16.81 mmol). The reaction was stirred for 16 h at 90° C. then diluted with water (200 mL). The resulting mixture was extracted with EtOAc (3×200 mL). The combined organic layers were dried over anhydrous Na₂SO₄. After filtration, the filtrate was concentrated under reduced pressure. The residue was purified by Prep-TLC (PE/EtOAc 1:1) to afford 350 mg (13%) of 4-(3-(4-(pyrazolo[1,5-a]pyridin-6-yl)-1H-imidazol-1-yl)propyl)morpholine as a white solid. ¹H NMR (400 MHZ, DMSO-d₆) δ (ppm) 8.91 (s, 1H), 7.96 (d, J=2.2 Hz, 1H), 7.79 (d, J=1.3 Hz, 1H), 7.76-7.67 (m, 2H), 7.61-7.58 (m, 1H), 6.59-6.56 (m, 1H), 4.03 (t, J=7.0 Hz, 2H), 3.59 (t, J=4.6 Hz, 4H), 2.38-2.31 (m, 4H), 2.26 (t, J=6.9 Hz, 2H), 1.93-1.90 (m, 2H). MS (ESI) m/z [M+H]⁺ calcd. for C₁₇H₂₁N₅O, 312.2; found, 312.3.

### Step 2. Preparation of 4-(3-(4-(3-bromopyrazolo[1,5-a]pyridin-6-yl)-1H-imidazol-1-yl)propyl)morpholine

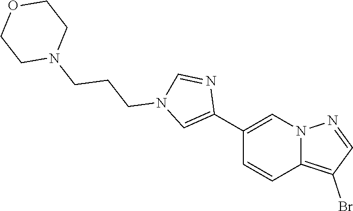 [SMILES: Brc1cnn2cc(-c3cn(CCCN4CCOCC4)cn3)ccc12]

To a solution of 4-(3-(4-(pyrazolo[1,5-a]pyridin-6-yl)-1H-imidazol-1-yl)propyl)morpholine (350 mg, 1.12 mmol, as prepared in the previous step) in DCM (5 mL) was added NBS (160 mg, 0.90 mmol). The reaction was stirred for 1 h at rt then concentrated under reduced pressure. The crude product was purified by reverse phase flash chromatography to afford 250 mg (57%) of 4-(3-(4-(3-bromopyrazolo[1,5-a]pyridin-6-yl)-1H-imidazol-1-yl)propyl)morpholine as a white solid. ¹H NMR (400 MHZ, DMSO-d₆) δ (ppm) 8.95 (s, 1H), 8.12 (s, 1H), 7.85 (s, 1H), 7.81-7.73 (m, 2H), 7.61 (d, J=9.2 Hz, 1H), 4.04 (t, J=7.0 Hz, 2H), 3.59 (t, J=4.6 Hz, 4H), 2.34 (t, J=4.4 Hz, 4H), 2.26 (t, J=6.9 Hz, 2H), 1.93-1.90 (m, 2H). MS (ESI) m/z [M+H]⁺ calcd. for C₁₇H₂₀BrN₅O, 390.1; found, 390.2.

### Step 3. tert-butyl 4-(6-(1-(3-morpholinopropyl)-1H-imidazol-4-yl)pyrazolo[1,5-a]pyridin-3-yl)piperazine-1-carboxylate (Compound 231)

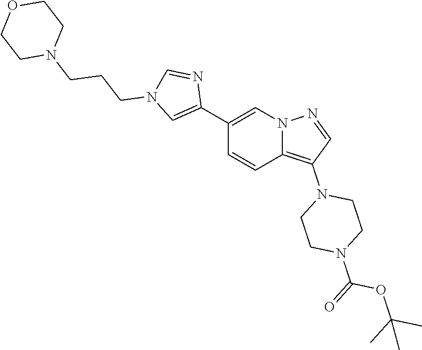 [SMILES: CC(C)(C)OC(=O)N1CCN(c2cnn3cc(-c4cn(CCCN5CCOCC5)cn4)ccc23)CC1]

To a solution of 4-(3-(4-(3-bromopyrazolo[1,5-a]pyridin-6-yl)-1H-imidazol-1-yl)propyl)morpholine (200 mg, 0.51 mmol, as prepared in the previous step) and tert-butyl piperazine-1-carboxylate (477 mg, 2.56 mmol) in dioxane (4 mL) and tBuOH (8 mL) was added KOtBu (86 mg, 0.77 mmol) and tBuXPhos Pd G1 (52 mg, 0.077 mmol). The reaction was stirred for 18 h at 80° C. then diluted with H₂O (100 mL). The resulting mixture was extracted with EtOAc (3×100 mL). The combined organic layers were dried over anhydrous Na₂SO₄. After filtration, the filtrate was concentrated under reduced pressure. The residue was purified by reverse phase flash to afford crude product. The crude product was purified by Prep-HPLC to afford 102.9 mg (40%) of tert-butyl 4-(6-(1-(3-morpholinopropyl)-1H-imidazol-4-yl)pyrazolo[1,5-a]pyridin-3-yl)piperazine-1-carboxylate (Compound 231) as a yellow semi-solid. ¹H NMR (400 MHZ, DMSO-d₆) δ (ppm) 8.76 (s, 1H), 7.81-7.74 (m, 2H), 7.72 (s, 1H), 7.65 (d, J=9.3 Hz, 1H), 7.47-7.45 (m, 1H), 4.03 (t, J=7.0 Hz, 2H), 3.59 (t, J=4.6 Hz, 4H), 3.51 (t, J=4.9 Hz, 4H), 2.95 (t, J=5.0 Hz, 4H), 2.34 (t, J=4.4 Hz, 4H), 2.25 (t, J=6.9 Hz, 2H), 1.93-1.90 (m, 2H), 1.43 (s, 9H). MS (ESI) m/z [M+H]⁺ calcd. for C₂₆H₃₇N₇O₃, 496.2; found, 496.3; HPLC purity: 254 nm: 99.6%.

Using the procedures described in Example, Compound and reagents, starting materials, and conditions known to those skilled in the art were used to prepare the compounds shown in Table CC.

TABLE CC

| Compound | Name | Characterization |
|---|---|---|
| Compound | tert-butyl 4-(6-(1-(3-morpholinopropyl)- | ¹H NMR (400 MHz, DMSO- |
| 232 | 1H-imidazol-5-yl)pyrazolo[1,5-a]pyridin- | d₆) δ (ppm) 8.69 (s, 1H), 7.89 |
| | 3-yl)piperazine-1-carboxylate | (s, 1H), 7.81 (d, J = 1.1 Hz, |
| | | 1H), 7.72 (d, J = 9.2 Hz, 1H), |
| | | 7.16-7.13 (m, 1H), 7.08 (d, J = |
| | | 1.1 Hz, 1H), 4.10 (t, J = 7.1 |
| | | Hz, 2H), 3.52 (t, J = 4.9 Hz, |
| | | 4H), 3.41 (m, 4H), 2.97 (t, J = |
| | | 5.0 Hz, 4H), 2.16-2.11 (m, |
| | | 6H), 1.73-1.70 (m, 2H), 1.44 |
| | | (s, 9H). MS (ESI) m/z |
| | | [M + H]⁺ calcd. for |
| | | C₂₆H₃₇N₇O₃, 496.3; found, |
| | | 496.3; HPLC purity: 254 nm: |
| | | 99.2%. |

### Example 173: Synthesis of Exemplary Compound 233

### tert-butyl 4-(6-(1-(3-morpholinopropyl)-1H-pyrazol-4-yl)pyrazolo[1,5-a]pyridin-3-yl)piperazine-1-carboxylate (Compound 233)

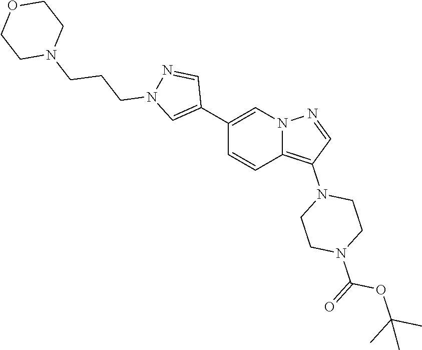 [SMILES: CC(C)(C)OC(=O)N1CCN(c2cnn3cc(-c4cnn(CCCN5CCOCC5)c4)ccc23)CC1]

### Step 1. Preparation of 6-(1H-pyrazol-4-yl)pyrazolo[1,5-a]pyridine

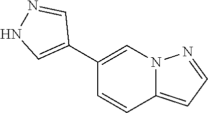 [SMILES: c1cc2ccc(-c3cn[nH]c3)cn2n1]

To a solution of 6-bromopyrazolo[1,5-a]pyridine (2 g, 10.15 mmol) and 4-(4,4,5,5-tetramethyl-1,3,2-dioxaborolan-2-yl)-1H-pyrazole (1.97 g, 10.15 mmol) in dioxane (90 mL) and H₂O (10 mL) were added Pd(dppf)Cl₂ (1.49 g, 2.03 mmol) and K₂CO₃ (2.81 g, 20.30 mmol). The reaction was stirred overnight at 90° C. under nitrogen then concentrated under reduced pressure. The residue was purified by silica gel column chromatography eluting with PE/EtOAc (1:1) to afford 600 mg (31%) of 6-(1H-pyrazol-4-yl)pyrazolo[1,5-a]pyridine as an off-white solid. MS (ESI) m/z [M+H]⁺ calcd. for C₁₀H₈N₄, 185.1; found, 185.2.

### Step 2. Preparation of 4-(3-(4-(pyrazolo[1,5-a]pyridin-6-yl)-1H-pyrazol-1-yl)propyl)morpholine

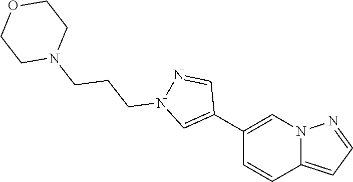 [SMILES: c1cc2ccc(-c3cnn(CCCN4CCOCC4)c3)cn2n1]

To a stirred solution of 6-(1H-pyrazol-4-yl)pyrazolo[1,5-a]pyridine (530 mg, 2.88 mmol, as prepared in the previous step) and 4-(3-chloropropyl)morpholine (565 mg, 3.45 mmol) in DMF (10 mL) was added Cs₂CO₃ (1874.9 mg, 5.75 mmol) at rt. The reaction was stirred overnight at 80° C. then allowed to cool to rt and quenched by the addition of water (10 mL). The resulting mixture was extracted with EtOAc (3×20 mL). The combined organic layers were washed with brine (3×5 mL) and dried over anhydrous Na₂SO₄. After filtration, the filtrate was concentrated under reduced pressure. The residue was purified by Prep-TLC (PE/EtOAc 1:1) to afford 300 mg (33%) of 4-(3-(4-(pyrazolo[1,5-a]pyridin-6-yl)-1H-pyrazol-1-yl)propyl)morpholine as an off-white solid. ¹H NMR (400 MHZ, DMSO-d₆) δ (ppm) δ 8.99 (s, 1H), 8.29 (s, 1H), 8.03-7.93 (m, 2H), 7.74-7.70 (m, 1H), 7.51-7.46 (m, 1H), 6.59 (d, J=2.3 Hz, 1H), 4.24-4.15 (m, 2H), 3.60-3.57 (m, 4H), 2.37-2.25 (m, 6H), 2.01-1.95 (m, 2H). MS (ESI) m/z [M+H]⁺ calcd. for C₁₇H₂₁N₅O, 312.2; found, 312.4.

### Step 3. Preparation of 4-(3-(4-(3-bromopyrazolo[1,5-a]pyridin-6-yl)-1H-pyrazol-1-yl)propyl)morpholine

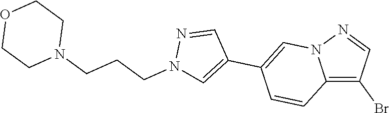 [SMILES: Brc1cnn2cc(-c3cnn(CCCN4CCOCC4)c3)ccc12]

A solution of 4-(3-(4-(pyrazolo[1,5-a]pyridin-6-yl)-1H-pyrazol-1-yl)propyl)morpholine (280 mg, 0.90 mmol, as prepared in the previous step) and NBS (176.1 mg, 0.99 mmol) in DCM (10 mL) was stirred for 4 h at rt under air atmosphere. The reaction was quenched by the addition of water (10 mL) at rt and extracted with DCM (3×20 mL). The combined organic layers were washed with brine (3×5 mL) and dried over anhydrous Na₂SO₄. After filtration, the filtrate was concentrated under reduced pressure to afford 220 mg (61%) of 4-(3-(4-(3-bromopyrazolo[1,5-a]pyridin-6-yl)-1H-pyrazol-1-yl)propyl)morpholine as an off-white solid. ¹H NMR (400 MHz, DMSO-d₆) δ (ppm) 9.07 (s, 1H), 8.34 (s, 1H), 8.13 (s, 1H), 8.04 (s, 1H), 7.66-7.59 (m, 2H), 4.17 (t, J=7.2 Hz, 2H), 3.58 (t, J=4.4 Hz, 4H), 2.34 (s, 4H), 2.28 (t, J=6.8 Hz, 2H), 1.99-1.96 (m, 2H). MS (ESI) m/z [M+H]⁺ calcd. for C₁₇H₂₀BrN₅O, 390.1; found, 390.3.

### Step 4. tert-butyl tert-butyl 4-(6-(1-(3-morpholinopropyl)-1H-pyrazol-4-yl)pyrazolo[1,5-a]pyridin-3-yl)piperazine-1-carboxylate (Compound 233)

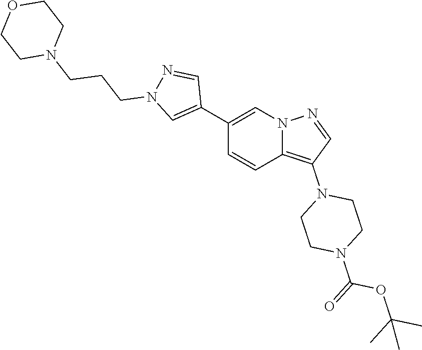 [SMILES: CC(C)(C)OC(=O)N1CCN(c2cnn3cc(-c4cnn(CCCN5CCOCC5)c4)ccc23)CC1]

To a solution of 4-(3-(4-(3-bromopyrazolo[1,5-a]pyridin-6-yl)-1H-pyrazol-1-yl)propyl)morpholine (200 mg, 0.512 mmol, as prepared in the previous step) and tert-butyl piperazine-1-carboxylate (477.3 mg, 2.56 mmol) in dioxane (4 mL) and tBuOH (8 mL) were added KOtBu (115 mg, 1.02 mmol) and tBuXPhos Pd G1 (70.4 mg, 0.10 mmol). The reaction was stirred overnight at 80° C. under nitrogen then concentrated under reduced pressure. The residue was purified by silica gel column chromatography eluting with DCM/MeOH (10:1) to afford the crude product. The crude product was purified by Prep-HPLC to afford 65.4 mg (25%) of tert-butyl 4-(6-(1-(3-morpholinopropyl)-1H-pyrazol-4-yl)pyrazolo[1,5-a]pyridin-3-yl)piperazine-1-carboxylate (Compound 233) as a light green solid. ¹H NMR (400 MHZ, DMSO-d₆) δ (ppm) 8.84 (s, 1H), 8.28 (s, 1H), 7.99 (s, 1H), 7.78 (s, 1H), 7.65 (d, J=9.2 Hz, 1H), 7.34 (d, J=9.6 Hz, 1H), 4.16 (t, J=6.8 Hz, 2H), 3.56 (t, J=4.4 Hz, 4H), 3.53-3.33 (m, 4H), 2.95 (t, J=9.6 Hz, 4H), 2.34 (s, 4H), 2.27 (t, J=7.2 Hz, 2H), 1.99-1.95 (m, 2H), 1.43 (s, 9H). MS (ESI) m/z [M+H]⁺ calcd. for C₂₆H₃₇N₇O₃, 496.3; found, 496.35; HPLC purity: 254 nm: 98.6%.

### Example 174: Synthesis of Exemplary Compound 234

### tert-butyl 4-(6-(1-isopropyl-1H-pyrazol-4-yl)pyrazolo[1,5-a]pyridin-3-yl)piperazine-1-carboxylate (Compound 234)

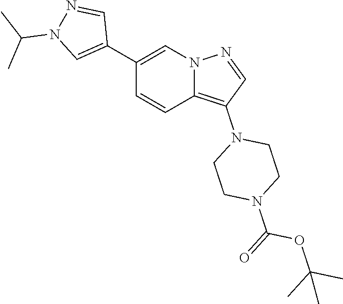 [SMILES: CC(C)n1cc(-c2ccc3c(N4CCN(C(=O)OC(C)(C)C)CC4)cnn3c2)cn1]

### Step 1. Preparation of 6-(1-isopropyl-1H-pyrazol-4-yl)pyrazolo[1,5-a]pyridine

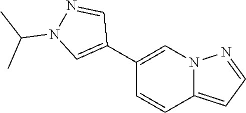 [SMILES: CC(C)n1cc(-c2ccc3ccnn3c2)cn1]

To a mixture of 6-bromopyrazolo[1,5-a]pyridine (1 g, 5.08 mmol) and 1-isopropyl-4-(4,4,5,5-tetramethyl-1,3,2-dioxaborolan-2-yl)pyrazole (1.8 g, 7.61 mmol) in dioxane (50 mL) and H₂O (5 mL) was added Pd(dip)Cl₂ (0.56 g, 0.76 mmol) and K₃PO₄ (0.32 g, 1.52 mmol). The reaction was stirred overnight at 90° C. under nitrogen then allowed to cool to rt. The mixture was diluted with H₂O (20 mL) and extracted with EtOAc (3×100 mL). The mixture was purified by silica gel column chromatography eluting with PE/EtOAc (1:1) to afford 1 g (87%) of 6-(1-isopropyl-1H-pyrazol-4-yl)pyrazolo[1,5-a]pyridine as a yellow solid. ¹H NMR (400 MHZ, DMSO-d₆) δ (ppm) 9.03-8.97 (m, 1H), 8.35 (d, J=0.9 Hz, 1H), 7.98 (m, 2H), 7.70 (m, 1H), 7.50 (m, 1H), 6.58 (m, 1H), 4.50 (m, 1H), 1.44 (m, 6H). MS (ESI) m/z [M+H]⁺ calcd. for C₁₃H₁₄N₄, 227.1; found, 227.2.

### Step 2. Preparation of 3-bromo-6-(1-isopropyl-1H-pyrazol-4-yl)pyrazolo[1,5-a]pyridine

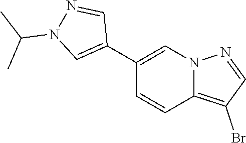 [SMILES: CC(C)n1cc(-c2ccc3c(Br)cnn3c2)cn1]

A mixture of 6-(1-isopropyl-1H-pyrazol-4-yl)pyrazolo[1,5-a]pyridine (900 mg, 3.98 mmol, as prepared in the previous step) in DCM (20 mL) was added NBS (566.3 mg, 3.18 mmol). The reaction was stirred for 2 h at rt then concentrated under reduced pressure. The residue was purified by Prep-TLC (PE/EtOAc 1:1) to afford 900 mg (74%) 3-bromo-6-(1-isopropyl-1H-pyrazol-4-yl)pyrazolo[1,5-a]pyridine as a yellow solid. ¹H NMR (400 MHZ, DMSO-d₆) δ (ppm) 9.07 (s, 1H), 8.40 (s, 1H), 8.12 (s, 1H), 8.03 (d, J=0.8 Hz, 1H), 7.66-7.60 (m, 2H), 4.50 (m, 1H), 1.46 (m, 6H); MS (ESI) m/z [M+H]⁺ calcd. for C₁₃H₁₃BrN₄, 305.0; found, 305.2.

### Step 3. tert-butyl 4-(6-(1-isopropyl-1H-pyrazol-4-yl)pyrazolo[1,5-a]pyridin-3-yl)piperazine-1-carboxylate (Compound 234)

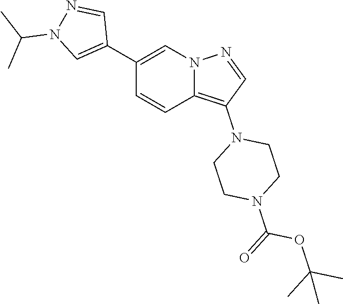 [SMILES: CC(C)n1cc(-c2ccc3c(N4CCN(C(=O)OC(C)(C)C)CC4)cnn3c2)cn1]

To a mixture of 3-bromo-6-(1-isopropyl-1H-pyrazol-4-yl)pyrazolo[1,5-a]pyridine (300 mg, 0.98 mmol, as prepared in the previous step) and tert-butyl piperazine-1-carboxylate (915.5 mg, 4.92 mmol) in tBuOH (6 mL) and dioxane (3 mL) was added KOtBu (165.5 mg, 1.47 mmol) and tBuXPhos Pd G3 (117.1 mg, 0.15 mmol). The reaction was stirred overnight at 90° C. under nitrogen, allowed to cool to rt. The mixture was purified by silica gel column chromatography eluting with PE/EtOAc (1:1) to afford 120 mg crude product. The crude product was purified by Prep-HPLC to afford 94 mg (23%) of tert-butyl 4-[6-(1-isopropylpyrazol-4-yl)pyrazolo[1,5-a]pyridin-3-yl]piperazine-1-carboxylate (Compound 234) as a white solid. ¹H NMR (400 MHZ, DMSO-d₆) δ (ppm) 8.84 (s, 1H), 8.34 (d, J=0.8 Hz, 1H), 7.98 (d, J=0.8 Hz, 1H), 7.77 (s, 1H), 7.64 (m, 1H), 7.36 (m, 1H), 4.50 (m, 1H), 3.51 (t, J=4.9 Hz, 4H), 2.95 (t, J=5.0 Hz, 4H), 1.49-1.45 (m, 6H), 1.45-1.43 (m, 9H). MS (ESI) m/z [M+H]⁺ calcd. for C₂₂H₃₀N₆O₂, 411.2; found, 411.1. HPLC purity: 254 nm: 99.5%.

Using the procedures described in Example 174, Compound 234 and reagents, starting materials, and conditions known to those skilled in the art were used to prepare the compounds shown in Table DD.

TABLE DD

| Compound | Name | Characterization |
|---|---|---|
| Compound | tert-butyl 4-{6-[1-(oxan-4-yl)pyrazol-4- | ¹H NMR (400 MHz, DMSO- |
| 235 | yl]pyrazolo[1,5-a]pyridin-3-yl}piperazine- | d₆) δ (ppm) 8.86 (s, 1H), 8.39 |
| | 1-carboxylate | (d, J = 2.8 Hz, 1H), 8.02 (d, J = |
| | | 2.8 Hz, 1H), 7.77 (d, J = 3.0 |
| | | Hz, 1H), 7.65 (m, 1H), 7.37- |
| | | 7.35 (d, J = 8.8 Hz, 1H), 4.46- |
| | | 4.36 (m, 1H), 4.02-3.94 |
| | | (m, 2H), 3.50-3.31 (m, 6H), |
| | | 2.98-2.91 (m, 4H), 2.00 (m, |
| | | 4H), 1.43 (m, 9H). MS (ESI) |
| | | m/z [M + H]⁺ calcd. for |
| | | C₂₄H₃₂N₆O₂, 453.3; found, |
| | | 453.3. HPLC purity: 254 nm: |
| | | 99.5%. |

### Example 175: Synthesis of Exemplary Compound 236

### tert-butyl 4-(4,5,6,7-tetrahydro-[3,6′-bipyrazolo[1,5-a]pyridin]-3′-yl)piperazine-1-carboxylate (Compound 236)

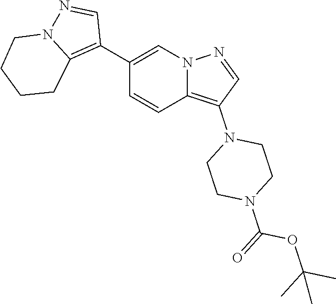 [SMILES: CC(C)(C)OC(=O)N1CCN(c2cnn3cc(-c4cnn5c4CCCC5)ccc23)CC1]

### Step 1. Preparation of 4,5,6,7-tetrahydro-3,6′-bipyrazolo[1,5-a]pyridine

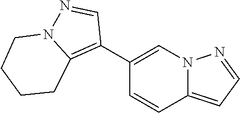 [SMILES: c1cc2ccc(-c3cnn4c3CCCC4)cn2n1]

To a solution of 6-bromopyrazolo[1,5-a]pyridine (600 mg, 3.05 mmol) and 3-(4,4,5,5-tetramethyl-1,3,2-dioxaborolan-2-yl)-4H,5H,6H,7H-pyrazolo[1,5-a]pyridine (756 mg, 3.05 mmol) in dioxane (10 mL) and H₂O (1.0 mL) was added XPhos Pd G3 (258 mg, 0.305 mmol), XPhos (145 mg, 0.31 mmol) and K₃PO₄ (1.29 g, 6.09 mmol) under nitrogen. The reaction was stirred for 16 h at 90° C. under nitrogen. The resulting mixture was filtered, and the filter cake was washed with EtOAc (2×5 mL). The filtrate was concentrated under reduced pressure. The residue was purified by silica gel column chromatography eluting with PE/EtOAc (1:2) to afford 720 mg (89%) of 4,5,6,7-tetrahydro-3,6′-bipyrazolo[1,5-a]pyridine as a yellow green solid. ¹H NMR (400 MHZ, DMSO-d₆) δ (ppm) 8.66 (s, 1H), 7.98 (d, J=2.2 Hz, 1H), 7.82 (s, 1H), 7.72 (d, J=9.2 Hz, 1H), 7.38 (d, J=9.0 Hz, 1H), 6.60 (d, J=2.2 Hz, 1H), 4.12 (t, J=6.1 Hz, 2H), 2.97 (t, J=6.3 Hz, 2H), 2.06-1.95 (m, 2H), 1.90-1.80 (m, 2H). MS (ESI) m/z [M+H]⁺ calcd. for C₁₄H₁₄N₄, 239.1; found, 239.2.

### Step 2. Preparation of 3′-bromo-4,5,6,7-tetrahydro-3,6′-bipyrazolo[1,5-a]pyridine

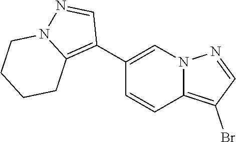 [SMILES: Brc1cnn2cc(-c3cnn4c3CCCC4)ccc12]

To a solution of 4,5,6,7-tetrahydro-3,6′-bipyrazolo[1,5-a]pyridine (700 mg, 2.94 mmol, as prepared in the previous step) in DCM (2.0 mL) was added NBS (575 mg, 3.23 mmol). The reaction was stirred for 2 h at rt then concentrated under reduced pressure. The residue was purified by silica gel column chromatography eluting with PE/EtOAc (3:1) to afford 850 mg (64%) of 3′-bromo-4,5,6,7-tetrahydro-3,6′-bipyrazolo[1,5-a]pyridine as a brown solid. ¹H NMR (400 MHZ, DMSO-d₆) δ (ppm) 8.72 (s, 1H), 8.13 (s, 1H), 7.84 (s, 1H), 7.60 (d, J=9.2 Hz, 1H), 7.53 (d, J=9.2 Hz, 1H), 4.12 (t, J=6.1 Hz, 2H), 2.97 (t, J=6.3 Hz, 2H), 2.05-1.94 (m, 2H), 1.90-1.79 (m, 2H). MS (ESI) m/z [M+H]⁺ calcd. for C₁₄H₁₃BrN₄, 317.0; found, 317.1.

### Step 3. Preparation of tert-butyl 4-(4,5,6,7-tetrahydro-[3,6′-bipyrazolo[1,5-a]pyridin]-3′-yl)piperazine-1-carboxylate (Compound 236)

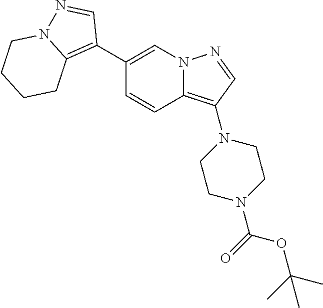 [SMILES: CC(C)(C)OC(=O)N1CCN(c2cnn3cc(-c4cnn5c4CCCC5)ccc23)CC1]

To a solution of 3′-bromo-4,5,6,7-tetrahydro-3,6′-bipyrazolo[1,5-a]pyridine (700 mg, 2.21 mmol, as prepared in the previous step) and tert-butyl piperazine-1-carboxylate (493 mg, 2.65 mmol) in tBuOH (11 mL) and dioxane (3.0 mL) was added KOtBu (372 mg, 3.31 mmol) and tBuXPhos Pd G1 (227 mg, 0.33 mmol) under nitrogen. The reaction was stirred for 60 h at 90° C. under nitrogen. The resulting mixture was filtered, the filter cake was washed with EtOAc (2×5 mL). The filtrate was concentrated under reduced pressure. The residue was purified by silica gel column chromatography eluting with PE/EtOAc (1:1). The crude product (400 mg) was purified by Prep-HPLC to afford 86.9 mg (9%) of tert-butyl 4-(4,5,6,7-tetrahydro-[3,6′-bipyrazolo[1,5-a]pyridin]-3′-yl)piperazine-1-carboxylate (Compound 236) as a yellow solid. ¹H NMR (400 MHZ, DMSO-d₆) δ (ppm) 8.50 (s, 1H), 7.79 (d, J=4.5 Hz, 2H), 7.67 (d, J=9.2 Hz, 1H), 7.24 (d, J=9.3 Hz, 1H), 4.11 (t, J=6.0 Hz, 2H), 3.50 (d, J=5.1 Hz, 4H), 3.00-2.90 (m, 6H), 1.99 (d, J=5.7 Hz, 2H), 1.84 (s, 2H), 1.43 (s, 9H). MS (ESI) m/z [M+H]⁺ calcd. for C₂₃H₃₀N₆O₂, 423.2; found, 423.2; HPLC purity: 254 nm: 98.4%.

### Example 176: Synthesis of Exemplary Compound 237

### tert-butyl 4-(6-(2-methyl-2H-tetrazol-5-yl)pyrazolo[1,5-a]pyridin-3-yl)piperazine-1-carboxylate (Compound 237)

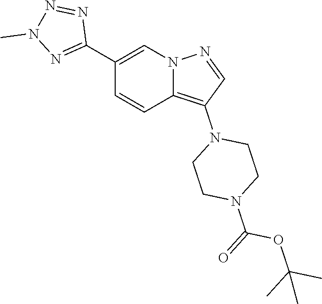 [SMILES: Cn1nnc(-c2ccc3c(N4CCN(C(=O)OC(C)(C)C)CC4)cnn3c2)n1]

### Step 1. Preparation of 6-(2-methyl-2H-tetrazol-5-yl)pyrazolo[1,5-a]pyridine

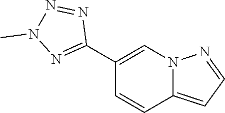

To a solution of 5-bromo-2-methyl-1,2,3,4-tetrazole (100 mg, 0.61 mmol), 6-(4,4,5,5-tetramethyl-1,3,2-dioxaborolan-2-yl)pyrazolo[1,5-a]pyridine (149.8 mg, 0.61 mmol), K₃PO₄ (260.5 mg, 1.23 mmol) in dioxane (5 mL) and H₂O (1 mL) was added Pd-PEPPSI-IPent^{Cl} 2-methylpyridine (51.6 mg, 0.06 mmol) at rt. The reaction was stirring at 90° C. for 5 h, then allowed to cool to rt. The resulting mixture was diluted with H₂O (15 mL), then was extracted with EtOAc (3×50 mL). The combined organic layers were dried over anhydrous Na₂SO₄. After filtration, the filtrate was concentrated under reduced pressure. Crude product was purified by Prep-TLC (PE/EtOAc 1:1) to afford 50 mg (41%) of 6-(2-methyl-2H-tetrazol-5-yl)pyrazolo[1,5-a]pyridine as a yellow solid. ¹H NMR (400 MHZ, DMSO-d₆) δ (ppm) 9.26 (s, 1H), 8.15 (s, 1H), 7.95-7.87 (m, 1H), 7.81-7.74 (m, 1H), 6.78-6.74 (m, 1H), 4.46 (s, 3H). MS (ESI) m/z [M+H]⁺ calcd. for C₉H₈N₆, 201.0; found, 201.1.

### Step 2. Preparation of 3-bromo-6-(2-methyl-2H-tetrazol-5-yl)pyrazolo[1,5-a]pyridine

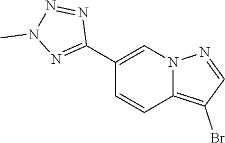 [SMILES: Cn1nnc(-c2ccc3c(Br)cnn3c2)n1]

To a solution of 6-(2-methyl-2H-tetrazol-5-yl)pyrazolo[1,5-a]pyridine (50 mg, 0.25 mmol, as prepared in the previous step) in DCM (5 mL) was added NBS (44.5 mg, 0.25 mmol) at rt. The reaction was stirred for 5 h, then diluted with H₂O (15 mL). The resulting mixture was extracted with EtOAc (3×50 mL) and the combined organic layers were dried over anhydrous Na₂SO₄. After filtration, the filtrate was concentrated under reduced pressure. The residue was purified by Prep-TLC (PE/EtOAc 1:1) to afford 25 mg (36%) of 3-bromo-6-(2-methyl-2H-tetrazol-5-yl)pyrazolo[1,5-a]pyridine as a yellow solid. ¹H NMR (400 MHZ, DMSO-d₆) δ (ppm) 9.31 (s, 1H), 8.32 (s, 1H), 7.91 (dd, J=9.2, 1.5 Hz, 1H), 7.79 (dd, J=9.3, 0.9 Hz, 1H), 4.47 (s, 3H). MS (ESI) m/z [M+H]⁺ calcd. for C₉H₇BrN₆, 279.0; found, 279.1.

### Step 3. Preparation of tert-butyl 4-(6-(2-methyl-2H-tetrazol-5-yl)pyrazolo[1,5-a]pyridin-3-yl)piperazine-1-carboxylate (Compound 237)

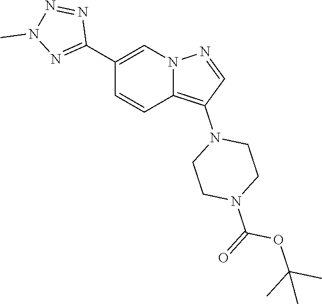 [SMILES: Cn1nnc(-c2ccc3c(N4CCN(C(=O)OC(C)(C)C)CC4)cnn3c2)n1]

To a solution of 3-bromo-6-(2-methyl-2H-tetrazol-5-yl)pyrazolo[1,5-a]pyridine (350 mg, 1.25 mmol, as prepared in the previous step), tert-butyl piperazine-1-carboxylate (1.17 g, 6.27 mmol), KOtBu (281.5 mg, 2.51 mmol) in tBuOH (16 mL) and dioxane (8 mL) was added tBuXPhos Pd G1 (88 mg, 0.13 mmol) at rt. The mixture was stirred at 90° C. for 18 h, then cooled to rt and diluted with H₂O (15 mL). The resulting mixture was extracted with EtOAc (3×50 mL) and the combined organic layers were dried over anhydrous Na₂SO₄. After filtration, the filtrate was concentrated under reduced pressure. The residue was purified by Prep-TLC (PE/EtOAc 1:1) and then crude product was purified by Prep-HPLC to afford 50.9 mg (11%) of tert-butyl 4-(6-(2-methyl-2H-tetrazol-5-yl)pyrazolo[1,5-a]pyridin-3-yl)piperazine-1-carboxylate (Compound 237) as a yellow solid. ¹H NMR (400 MHZ, DMSO-d₆) δ (ppm) 9.10 (s, 1H), 7.99 (s, 1H), 7.89-7.82 (m, 1H), 7.64-7.57 (m, 1H), 4.45 (s, 3H), 3.59-3.48 (m, 4H), 3.04-2.93 (m, 4H), 1.44 (s, 9H). MS (ESI) m/z [M+H]⁺ calcd. for C₁₈H₂₄N₈O₂, 485.2; found, 385.4; HPLC purity: 254 nm: 97.0%.

### Example 177: Synthesis of Exemplary Compound 238

### tert-butyl-2-methyl-4-(6-(1-methyl-1H-pyrazol-4-yl)pyrazolo[1,5-a]pyridin-3-yl)piperazine-1-carboxylate (Compound 238)

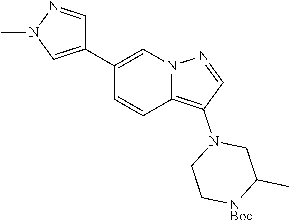

To a solution of 3-bromo-6-(1-methyl-1H-pyrazol-4-yl)pyrazolo[1,5-a]pyridine (Compound S9) (1.00 g, 3.61 mmol) dissolved in dioxane (5 mL) and tBuOH (5 mL) was added tert-butyl 2-methylpiperazine-1-carboxylate (1.08 g, 5.41 mmol), KOtBu (607 mg, 5.41 mmol) and tBuXPhos Pd G1 (372 mg, 0.54 mmol). The resulting solution was stirred 90° C. for 16 hours under nitrogen then allowed to cool to rt. The resulting mixture was diluted with water (20 mL) and extracted with EtOAc (3×40 mL). The organic layers were combined was concentrated. The crude product was purified by Prep-HPLC to afford 136.4 mg (9%) of tert-butyl-2-methyl-4-(6-(1-methyl-1H-pyrazol-4-yl)pyrazolo[1,5-a]pyridin-3-yl)piperazine-1-carboxylate (Compound 238) as a yellow solid. ¹H NMR (400 MHZ, DMSO-d₆) δ (ppm) 8.84 (s, 1H), 8.23 (s, 1H), 7.98 (s, 1H), 7.76 (s, 1H), 7.58 (d, J=9.2 Hz, 1H), 7.34 (d, J=9.3 Hz, 1H), 4.22 (m, 1H), 3.87 (s, 3H), 3.85-3.79 (m, 1H), 3.28-3.21 (m, 2H), 3.11 (d, J=11.5 Hz, 1H), 2.77 (d, J=3.8 Hz, 1H), 2.70-2.59 (m, 1H), 1.43 (s, 9H), 1.34 (d, J=6.7 Hz, 3H). MS (ESI) m/z [M+H]⁺ calcd. for C₂₁H₁₈N₆O₂, 397.2; found, 397.2; HPLC purity: 254 nm: 97.0%.

### Example 178: Synthesis of Exemplary Compound 239

### 3-(4-(1-benzyl-1H-1,2,4-triazol-5-yl)piperazin-1-yl)-6-(1-methyl-1H-pyrazol-4-yl)pyrazolo[1,5-a]pyridine (Compound 239)

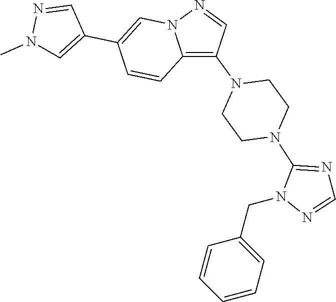 [SMILES: Cn1cc(-c2ccc3c(N4CCN(c5ncnn5Cc5ccccc5)CC4)cnn3c2)cn1]

### Step 1. Preparation of 1-benzyl-5-chloro-1H-1,2,4-triazole

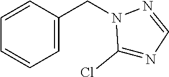 [SMILES: Clc1ncnn1Cc1ccccc1]

To a solution of 3-chloro-1H-1,2,4-triazole (950 mg, 9.18 mmol) and (bromomethyl)benzene (1.88 g, 11.01 mmol) in DMF (10 mL) were added K₂CO₃ (2.54 g, 18.36 mmol). The reaction was stirred for 4 h at 80° C. under nitrogen then allowed to cool to rt. Then resulting mixture was filtered and the filter cake was washed with DCM (3×50 mL). The filtrate was concentrated under reduced pressure. The residue was purified by reversed-phase flash chromatography to afford 120 mg (11%) of 1-benzyl-5-chloro-1H-1,2,4-triazole as a light-yellow oil. MS (ESI) m/z [M+H]⁺ calcd. for C₉H₈ClN₃, 194.0; found, 194.1.

### Step 2. 3-(4-(1-benzyl-1H-1,2,4-triazol-5-yl)piperazin-1-yl)-6-(1-methyl-1H-pyrazol-4-yl)pyrazolo[1,5-a]pyridine (Compound 239)

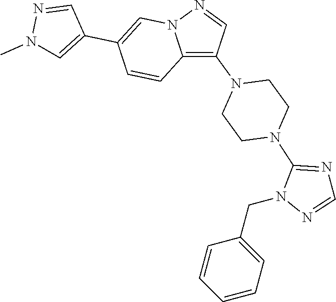 [SMILES: Cn1cc(-c2ccc3c(N4CCN(c5ncnn5Cc5ccccc5)CC4)cnn3c2)cn1]

To a solution of 1-benzyl-5-chloro-1,2,4-triazole (100 mg, 0.51 mmol, as prepared in the previous step) and 1-[6-(1-methylpyrazol-4-yl)pyrazolo[1,5-a]pyridin-3-yl]piperazine (Compound 12) (29.1 mg, 0.10 mmol) in dioxane (3 mL) were added Cs₂CO₃ (336.5 mg, 1.03 mmol) and Pd-PEPPSI-IPent^{Cl} 2-methylpyridine (86.8 mg, 0.10 mmol). The reaction was stirred for 16 h at 90° C. under nitrogen then concentrated under reduced pressure. The residue was diluted with water and extracted with EtOAc (3×50 mL). The combined organic layers were washed with brine (10 mL) and dried over anhydrous Na₂SO₄. After filtration, the filtrate was concentrated under reduced pressure. The crude product was purified by Prep-HPLC to afford 4.7 mg (2%) of 3-(4-(1-benzyl-1H-1,2,4-triazol-5-yl)piperazin-1-yl)-6-(1-methyl-1H-pyrazol-4-yl)pyrazolo[1,5-a]pyridine (Compound 239) as a brown semi-solid. ¹H NMR (400 MHz, DMSO-d₆) δ (ppm) 8.83 (s, 1H), 8.23 (s, 1H), 7.98 (s, 1H), 7.77 (d, J=4.3 Hz, 2H), 7.67 (d, J=9.2 Hz, 1H), 7.43-7.28 (m, 4H), 7.23 (d, J=7.4 Hz, 2H), 5.28 (s, 2H), 3.87 (s, 3H), 3.28-3.21 (m, 4H), 3.11 (t, J=4.8 Hz, 4H). MS (ESI) m/z [M+H]⁺ calcd. for C₂₄H₂₅N₉, 440.2; found, 440.2; HPLC purity: 254 nm: 98.0%.

### Example 179: Synthesis of Exemplary Compound 240

### tert-butyl 3-methyl-4-(6-(1-methyl-1H-pyrazol-4-yl)pyrazolo[1,5-a]pyridin-3-yl)piperazine-1-carboxylate (Compound 240)

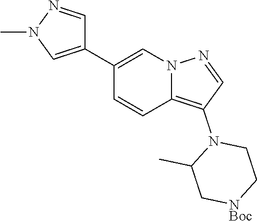 [SMILES: CC1CN(C(=O)OC(C)(C)C)CCN1c1cnn2cc(-c3cnn(C)c3)ccc12]

To a solution of 3-bromo-6-(1-methyl-1H-pyrazol-4-yl)pyrazolo[1,5-a]pyridine (Compound S9) (1.00 g, 3.61 mmol) dissolved in toluene (20.0 mL) was added tert-butyl 3-methylpiperazine-1-carboxylate (867 mg, 4.33 mmol), KOtBu (810 mg, 7.22 mmol), tBuXPhos (153 mg, 0.36 mmol) and tBuXPhos Pd G3 (287 mg, 0.36 mmol). The resulting solution was stirred at 90° C. for 16 hours under then allowed to cool down to rt. The resulting mixture was concentrated under reduced pressure. The residue was applied onto a silica gel column with EtOAc/PE (1/1). The residue was purified by reverse phase chromatography to afford 35.2 mg (2%) of tert-butyl-3-methyl-4-(6-(1-methyl-1H-pyrazol-4-yl)pyrazolo[1,5-a]pyridin-3-yl)piperazine-1-carboxylate (Compound 240) as a light yellow oil. ¹H NMR (400 MHz, DMSO-d₆) δ (ppm) 8.83 (s, 1H), 8.21 (s, 1H), 7.96 (s, 1H), 7.90 (s, 1H), 7.59 (d, J=9.2 Hz, 1H), 7.36 (d, J=9.3 Hz, 1H), 3.87 (s, 3H), 3.81-3.67 (m, 2H), 3.25-3.20 (m, 1H), 3.11-2.85 (m, 4H), 1.44 (s, 9H), 0.81 (d, J=6.0 Hz, 3H). MS (ESI) m/z [M+H]⁺ calcd. for C₂₁H₂₈N₆O₂, 397.2; found, 397.15; HPLC purity: 254 nm: 99.1%.

### Example 180: Synthesis of Exemplary Compound 241

### (5-(4-(6-(1-methyl-1H-pyrazol-4-yl)pyrazolo[1,5-a]pyridin-3-yl)piperazin-1-yl)pyridin-2-yl)(phenyl)methanol (Compound 241)

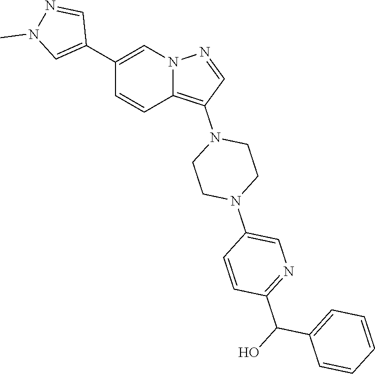 [SMILES: Cn1cc(-c2ccc3c(N4CCN(c5ccc(C(O)c6ccccc6)nc5)CC4)cnn3c2)cn1]

### Step 1. Preparation of (5-bromopyridin-2-yl)(phenyl)methanol

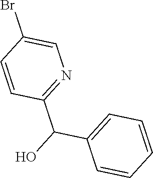 [SMILES: OC(c1ccccc1)c1ccc(Br)cn1]

To a solution of 5-bromopyridine-2-carbaldehyde (2 g, 10.75 mmol) in THF (25 mL) was added PhMgBr (1.95 g, 10.75 mmol) dropwise at 0° C. under nitrogen. at the reaction was stirred overnight at rt then quenched with sat. aq. NH₄Cl at 0° C. The resulting mixture was concentrated under vacuum. The residue was purified by silica gel column chromatography eluting with PE/EtOAc (3:1) to afford 2.69 g (86%) of (5-bromopyridin-2-yl)(phenyl)methanol as a yellow oil. ¹H NMR (400 MHZ, DMSO-d₆) δ (ppm) 8.58 (s, 1H), 8.04 (m, 1H), 7.57 (m, 1H), 7.39 (m, 2H), 7.30 (m, 2H), 7.28-7.18 (m, 1H), 6.21 (d, J=4.5 Hz, 1H), 5.70 (d, J=3.4 Hz, 1H). MS (ESI) m/z [M+H]⁺ calcd. for C₁₂H₁₀BrNO, 264.0; found 264.0.

### Step 2. (5-(4-(6-(1-methyl-1H-pyrazol-4-yl)pyrazolo[1,5-a]pyridin-3-yl)piperazin-1-yl)pyridin-2-yl)(phenyl)methanol (Compound 241)

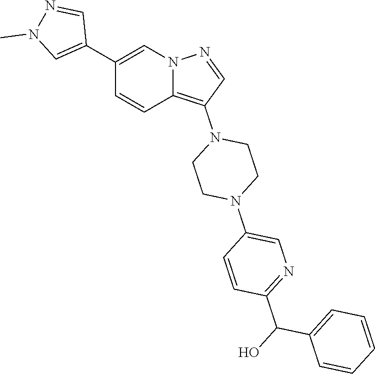 [SMILES: Cn1cc(-c2ccc3c(N4CCN(c5ccc(C(O)c6ccccc6)nc5)CC4)cnn3c2)cn1]

To a solution of (5-bromopyridin-2-yl)(phenyl)methanol (600 mg, 2.27 mmol, as prepared in the previous step) and 1-[6-(1-methylpyrazol-4-yl)pyrazolo[1,5-a]pyridin-3-yl]piperazine (Compound 12) (641.4 mg, 2.27 mmol) in dioxane (20 mL) were added CPhos Pd G3 (280.2 mg, 0.34 mmol), CPhos (148.8 mg, 0.34 mmol), and Cs₂CO₃ (1.48 g, 4.54 mmol). The reaction was stirred overnight at 90° C. under nitrogen then allowed to cool to rt. The resulting mixture was concentrated under reduced pressure. The crude product was purified by Prep-HPLC to afford 64.3 mg (6%) of (5-(4-(6-(1-methyl-1H-pyrazol-4-yl)pyrazolo[1,5-a]pyridin-3-yl)piperazin-1-yl)pyridin-2-yl)(phenyl)methanol (Compound 241) as a yellow solid. ¹H NMR (400 MHZ, DMSO-d₆) δ (ppm) 8.84 (s, 1H), 8.23 (s, 2H), 7.98 (s, 1H), 7.80 (d, J=2.2 Hz, 1H), 7.68 (d, J=9.2 Hz, 1H), 7.46-7.15 (m, 8H), 5.90 (s, 1H), 5.65 (s, 1H), 3.87 (s, 3H), 3.44-3.33 (m, 4H), 3.15 (t, J=4.8 Hz, 4H). MS (ESI) m/z [M+H]⁺ calcd. for C₂₇H₂₇N₇O, 466.2; found, 466.2; HPLC purity: 254 nm: 99.2%.

### Example 181: Synthesis of Exemplary Compound 242

### 6-(1-methyl-1H-pyrazol-4-yl)-3-(4-phenethylcyclohex-1-en-1-yl)pyrazolo[1,5-a]pyridine (Compound 242)

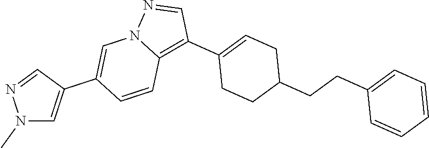 [SMILES: Cn1cc(-c2ccc3c(C4=CCC(CCc5ccccc5)CC4)cnn3c2)cn1]

To a solution of 4-(2-phenylethyl)cyclohex-1-en-1-yl trifluoromethanesulfonate (Compound S76) (200 mg, 0.6 mmol) in dioxane (10 mL) and H₂O (1 mL) was added 1-methyl-4-[3-(4,4,5,5-tetramethyl-1,3,2-dioxaborolan-2-yl)pyrazolo[1,5-a]pyridin-6-yl]pyrazole (Compound S57) (213 mg, 0.66 mmol), K₂CO₃ (165 mg, 1.2 mmol) and Pd(dppf)Cl₂ (43 mg, 0.06 mmol). The reaction was stirred overnight at 90° C. under nitrogen, H₂O (10 mL) was added, then extracted with EtOAc (3×50 mL). The mixture was purified by Prep-TLC (PE/EtOAc 1:1). The crude product was purified by Prep-HPLC to afford 27.8 mg (12%) of 6-(1-methyl-1H-pyrazol-4-yl)-3-(4-phenethylcyclohex-1-en-1-yl)pyrazolo[1,5-a]pyridine (Compound 242) as a white solid. ¹H NMR (400 MHZ, DMSO-d₆) δ (ppm) 8.93 (s, 1H), 8.24 (s, 1H), 8.00 (d, J=8.9 Hz, 2H), 7.82 (d, J=9.3 Hz, 1H), 7.46 (m, 1H), 7.33-7.21 (m, 4H), 7.21-7.13 (m, 1H), 6.07 (s, 1H), 3.87 (s, 3H), 2.67 (t, J=7.5 Hz, 2H), 2.41 (m, 1H), 2.36 (m, 2H), 1.92 (m, 2H), 1.61 (m, 3H), 1.40 (m, 1H). MS (ESI) m/z [M−H]⁺ calcd. for C₂₅H₂₆N₄, 383.2; found, 383.1. HPLC purity: 254 nm: 99.6%.

Using the procedures described in Example 181, Compound 242 and reagents, starting materials, and conditions known to those skilled in the art were used to prepare the compounds shown in Table EE.

TABLE EE

| Compound | Name | Characterization |
|---|---|---|
| Compound | 6-(1-methyl-1H-pyrazol-4-yl)-3-(4- | ¹H NMR (400 MHz, DMSO- |
| 243 | (phenoxymethyl)cyclohex-1-en-1- | d₆) δ (ppm) 8.94 (s, 1H), 8.25 |
| | yl)pyrazolo[1,5-a]pyridine | (s, 1H), 8.04 (s, 1H), 7.99 (s, |
| | | 1H), 7.85 (d, J = 9.2 Hz, 1H), |
| | | 7.47 (d, J = 9.2 Hz, 1H), 7.38- |
| | | 7.24 (m, 2H), 7.01-6.90 |
| | | (m, 3H), 6.12 (s, 1H), 4.06- |
| | | 3.81 (m, 5H), 2.62-2.54 (m, |
| | | 1H), 2.45-2.40 (m, 1H), 2.22- |
| | | 1.94 (m, 3H), 1.61-1.47 |
| | | (m, 1H), 1.23 (s, 1H). MS |
| | | (ESI) m/z [M + H]⁺ calcd. for |
| | | C₂₄H₂₄N₄O, 385.2; found, |
| | | 385.3. HPLC Purity: 254 nm: |
| | | 99.8%. |

### Example 182: Synthesis of Exemplary Compound 244

### N-(5-benzylpyrimidin-2-yl)-2-((6-(1-methyl-1H-pyrazol-4-yl)pyrazolo[1,5-a]pyridin-3-yl)amino) acetamide (Compound 244)

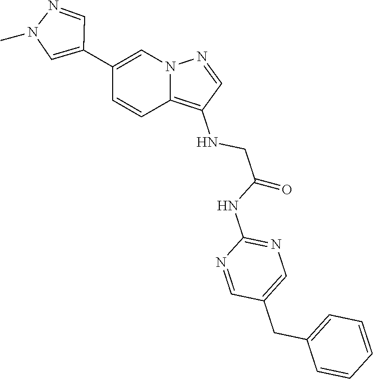 [SMILES: Cn1cc(-c2ccc3c(NCC(=O)Nc4ncc(Cc5ccccc5)cn4)cnn3c2)cn1]

### Step 1. Preparation of 5-benzylpyrimidin-2-amine

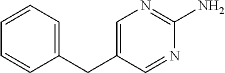 [SMILES: Nc1ncc(Cc2ccccc2)cn1]

To a solution of 30% aqueous NH₄OH (8 mL) was added 5-benzyl-2-chloropyrimidine (Compound S88) (200 mg, 0.978 mmol) at rt. The reaction was stirred at 90° C. for 18 h then diluted with H₂O (15 mL). The resulting mixture was extracted with EtOAc (3×50 mL). The combined organic layers were dried over anhydrous Na₂SO₄. After filtration, the filtrate was concentrated under reduced pressure. The residue was purified by Prep-TLC (PE/EtOAc 1:1) to afford 30 mg (17%) of 5-benzylpyrimidin-2-amine as a white solid. ¹H NMR (400 MHZ, DMSO-d₆) δ (ppm) 8.12 (s, 2H), 7.34-7.14 (m, 5H), 6.46 (s, 2H), 3.73 (s, 2H). MS (ESI) m/z [M+H]⁺ calcd. for C₁₁H₁₁N₃, 186.1; found, 186.1.

### Step 2. Preparation of N-(5-benzylpyrimidin-2-yl)-2-bromoacetamide

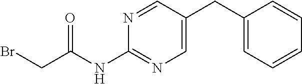 [SMILES: O=C(CBr)Nc1ncc(Cc2ccccc2)cn1]

To a solution of 5-benzylpyrimidin-2-amine (100 mg, 0.54 mmol, as prepared in the previous step), Et₃N (163.9 mg, 1.62 mmol) in DCM (10 mL) was added bromoacetyl bromide (119.9 mg, 0.59 mmol) at 0° C. The reaction was stirred at 0° C. for 2 h then diluted with H₂O (15 mL). The resulting mixture was extracted with EtOAc (3×50 mL). The combined organic layers were dried over anhydrous Na₂SO₄. After filtration, the filtrate was concentrated under reduced pressure. The residue was purified by Prep-TLC (PE/EtOAc 1:1) to afford 35 mg (21%) of N-(5-benzylpyrimidin-2-yl)-2-bromoacetamide as a yellow solid. ¹H NMR (400 MHz, DMSO-d₆) δ (ppm) 9.22 (s, 1H), 9.09 (s, 1H), 8.59 (d, J=5.8 Hz, 1H), 7.76-7.27 (m, 5H) 5.19 (s, 2H), 4.11 (s, 2H). MS (ESI) m/z [M+H]⁺ calcd. for C₁₃H₁₂BrN₃O, 306.0; found, 306.1.

### Step 3. Preparation of N-(5-benzylpyrimidin-2-yl)-2-((6-(1-methyl-1H-pyrazol-4-yl)pyrazolo[1,5-a]pyridin-3-yl)amino) acetamide (Compound 244)

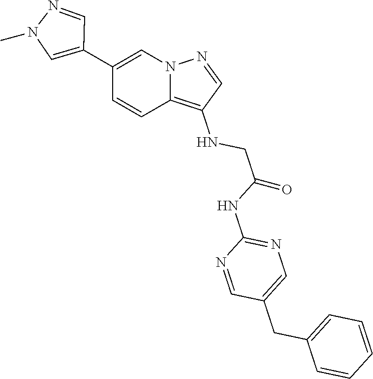 [SMILES: Cn1cc(-c2ccc3c(NCC(=O)Nc4ncc(Cc5ccccc5)cn4)cnn3c2)cn1]

To a mixture of 6-(1-methylpyrazol-4-yl)pyrazolo[1,5-a]pyridin-3-amine (Compound S56) (167.2 mg, 0.78 mmol), K₂CO₃ (325 mg, 2.35 mmol) in DMF (20 mL) was added N-(5-benzylpyrimidin-2-yl)-2-bromoacetamide (240 mg, 0.78 mmol, as prepared in the previous step) at 0° C. The reaction was stirred at rt for 5 h then diluted with H₂O (25 mL). The resulting mixture was extracted with EtOAc (3×50 mL). The combined organic layers were dried over anhydrous Na₂SO₄. After filtration, the filtrate was concentrated under reduced pressure. The residue was purified by Prep-TLC (PE/EtOAc 1:1) and the crude product was purified by Prep-HPLC to afford 20.4 mg (6%) of N-(5-benzylpyrimidin-2-yl)-2-((6-(1-methyl-1H-pyrazol-4-yl)pyrazolo[1,5-a]pyridin-3-yl)amino) acetamide (Compound 244) as a yellow solid. ¹H NMR (400 MHZ, DMSO-d₆) δ (ppm) 10.49 (s, 1H), 8.69 (s, 1H), 8.58 (d, J=8.7 Hz, 2H), 8.18 (s, 1H), 7.94 (s, 1H), 7.71-7.64 (m, 1H), 7.49 (s, 1H), 7.36-7.22 (m, 4H), 7.26-7.15 (m, 2H), 5.30 (t, J=6.7 Hz, 1H), 4.02 (d, J=6.5 Hz, 2H), 3.93 (s, 2H), 3.86 (s, 3H). MS (ESI) m/z [M+H]⁺ calcd. for C₂₄H₂₂N₈O, 439.2; found, 439.1; HPLC purity: 254 nm: 92.0%.

### Example 183: Synthesis of Exemplary Compound 245

### 3-benzyl-5-(4-(6-(1-methyl-1H-pyrazol-4-yl)pyrazolo[1,5-a]pyridin-3-yl)piperazin-1-yl)-1,2,4-thiadiazole (Compound 245)

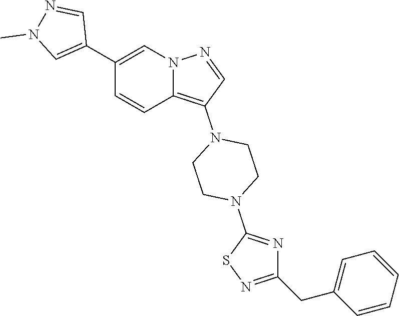 [SMILES: Cn1cc(-c2ccc3c(N4CCN(c5nc(Cc6ccccc6)ns5)CC4)cnn3c2)cn1]

### Step 1. Preparation of 3-benzyl-5-chloro-1,2,4-thiadiazole

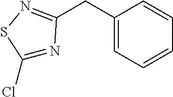 [SMILES: Clc1nc(Cc2ccccc2)ns1]

To a solution of 3-bromo-5-chloro-1,2,4-thiadiazole (500 mg, 2.51 mmol) and potassium benzyltrifluoroborate (347.5 mg, 1.76 mmol) in dioxane (20 mL) and H₂O (4 mL) were added tBu₃P Pd G3 (143.4 mg, 0.25 mmol), tBu₃PHBF₄ (72.7 mg, 0.25 mmol), and K₃PO₄ (1.06 g, 5.01 mmol). The reaction was stirred for 1 h at 80° C. under nitrogen then cooled to rt and concentrated under reduced pressure. The residue was dissolved in water (100 mL), extracted with EtOAc (3×100 mL) and dried over anhydrous Na₂SO₄. After filtration, the filtrate was concentrated under reduced pressure. The residue was purified by silica gel column chromatography eluting with PE/EtOAc (5:1) to afford 50 mg (10%) of 3-benzyl-5-chloro-1,2,4-thiadiazole as a yellow oil. MS (ESI) m/z [M+H]⁺ calcd. for C₉H₇ClN₂S, 211.0; found, 211.0.

### Step 2. 3-benzyl-5-(4-(6-(1-methyl-1H-pyrazol-4-yl)pyrazolo[1,5-a]pyridin-3-yl)piperazin-1-yl)-1,2,4-thiadiazole (Compound 245)

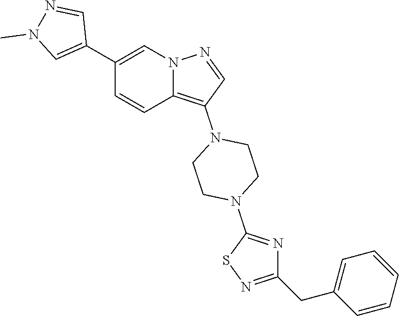 [SMILES: Cn1cc(-c2ccc3c(N4CCN(c5nc(Cc6ccccc6)ns5)CC4)cnn3c2)cn1]

To a solution of 3-benzyl-5-chloro-1,2,4-thiadiazole (50 mg, 0.24 mmol, as prepared in the previous step) and 1-[6-(1-methylpyrazol-4-yl)pyrazolo[1,5-a]pyridin-3-yl]piperazine (Compound 12) (67.0 mg, 0.24 mmol, as prepared in Example 15) in IPA (10 mL) was added DIPEA (61.4 mg, 0.47 mmol). The reaction was stirred for 4 days at 105° C. under nitrogen then allowed to cool to rt. The resulting mixture was concentrated under reduced pressure. The residue was dissolved in water (50 mL), extracted with EtOAc (3×50 mL), and dried over anhydrous Na₂SO₄. After filtration, the filtrate was concentrated under reduced pressure. The crude product was purified by Prep-HPLC to afford 5.5 mg (5%) of 3-benzyl-5-(4-(6-(1-methyl-1H-pyrazol-4-yl)pyrazolo[1,5-a]pyridin-3-yl)piperazin-1-yl)-1,2,4-thiadiazole (Compound 245) as a purple solid. ¹H NMR (400 MHZ, DMSO-d₆) δ (ppm) 8.84 (s, 1H), 8.24 (s, 1H), 7.98 (s, 1H), 7.80 (s, 1H), 7.68 (d, J=9.2 Hz, 1H), 7.43-7.14 (m, 6H), 3.99 (s, 2H), 3.88 (s, 3H), 3.66 (t, J=5.1 Hz, 4H), 3.11 (t, J=5.1 Hz, 4H). MS (ESI) m/z [M+H]⁺ calcd. for C₂₄H₂₄N₈S, 457.1; found, 457.1. HPLC purity: 254 nm: 95.0%.

### Example 184: Synthesis of Exemplary Compound 246

### 1-(4-fluorophenyl)-1-(2-{4-[6-(1-methyl-1H-pyrazol-4-yl)pyrazolo[1,5-a]pyridin-3-yl]piperazin-1-yl}pyrimidin-5-yl)methanamine (Compound 246)

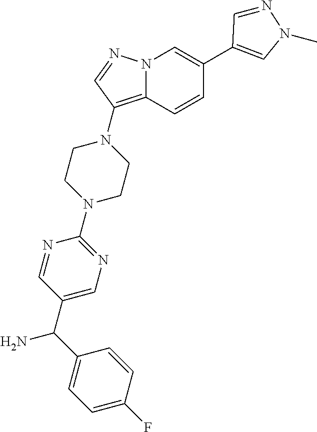 [SMILES: Cn1cc(-c2ccc3c(N4CCN(c5ncc(C(N)c6ccc(F)cc6)cn5)CC4)cnn3c2)cn1]

### Step 1. Preparation of N-[(1E)-(4-fluorophenyl)methylene]-2-methylpropane-2-sulfinamide

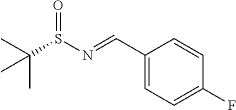

To a solution of(S)-2-methyl-propane-2-sulfinic acid amide (0.888 g, 7.32 mmol) in DCM (1 mL) was added CuSO₄ (dried by heating in a flask with a torch under vacuum then cooled under nitrogen, 2.572 g, 16.11 mmol) followed by a solution of 4-fluorobenzaldehyde (1.00 g, 8.06 mmol) in DCM (1.5 mL). After 16 h, additional CuSO₄ (2.0 g) and DCM (8 mL) were added, and the reaction was stirred an additional 20 h. The mixture was filtered through celite and the solids were extracted with DCM. The combined organic layers were concentrated under reduced pressure to afford an oil. The residue was purified by silica gel chromatography eluting with a gradient of 0-20% EtOAc in hexanes to afford 1.25 g, (75%) of N-[(1E)-(4-fluorophenyl)methylene]-2-methylpropane-2-sulfinamide as an oil: ¹H NMR (400 MHZ, CDCl₃) δ (ppm) 8.58 (s, 1H), 7.89 (dd, J=8.74, 5.44 Hz, 2H), 7.19 (t, J=8.62 Hz, 2H), 1.29 (s, 9H). MS (ESI) m/z [M+H]⁺ calcd. for C₁₁H₁₅FNOS, 228.1; found, 228.1.

### Step 2. Preparation of tert-butyl 4-{5-[{[(S)-tert-butylsulfinyl]amino}(4-fluorophenyl)methyl]pyrimidin-2-yl}piperazine-1-carboxylate

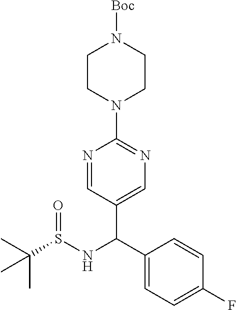 [SMILES: CC(C)(C)OC(=O)N1CCN(c2ncc(C(N[S@@](=O)C(C)(C)C)c3ccc(F)cc3)cn2)CC1]

To a solution of tert-butyl 4-(5-bromopyrimidin-2-yl)piperazine-1-carboxylate (0.500 g, 1.46 mmol) in THF (10 mL) cooled to −78° C. was slowly added 2M nBuLi in hexanes (0.801 mL, 1.60 mmol) over 10 min. After an additional 10 min, a solution of N-[(1E)-(4-fluorophenyl)methylene]-2-methylpropane-2-sulfinamide (0.364 g, 1.60 mmol, as prepared in the previous step) in THF (3.0 mL) was slowly added. After 1 h, the reaction was quenched by the addition of AcOH (0.207 mL, 3.64 mmol) in 3.0 mL hexanes. The dry ice bath was removed, and the reaction mixture treated sat. aq. NH₄Cl while warming to ambient temperature. The mixture was diluted with EtOAc, and the organic layer was washed with 0.1 N aq. HCl, sat. aq. NaHCO₃, and brine, then dried over anhydrous Na₂SO₄, and filtered. The filtrate was concentrated under reduced pressure. The residue was purified by silica gel column chromatography eluting with a gradient of 0-10% MeOH in DCM to afford 350 mg (61%) of tert-butyl 4-{5-[{[(S)-tert-butylsulfinyl]amino}(4-fluorophenyl)methyl]pyrimidin-2-yl}piperazine-1-carboxylate as a foam. ¹H NMR (400 MHZ, CDCl₃) δ (ppm) 8.34 (s, 2H), 7.32-7.41 (m, 2H), 7.07 (t, J=8.62 Hz, 2H), 5.50 (d, J=2.45 Hz, 1H), 3.75-3.87 (m, 4H), 3.65 (d, J=2.32 Hz, 1H), 3.45-3.56 (m, 4H), 1.47-1.53 (m, 9H), 1.26-1.31 (m, 9H). MS (ESI) m/z [M+H]⁺ calcd. for C₂₄H₃₅FN₅O₃S, 492.6; found, 492.4; HPLC purity: 210 nm: 100.0%; 254 nm: 100.0%.

### Step 3. Preparation of N-[(4-fluorophenyl)(2-piperazin-1-ylpyrimidin-5-yl)methyl]-2-methylpropane-2-sulfinamide

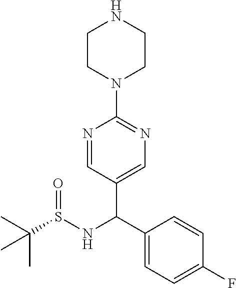 [SMILES: CC(C)(C)[S@](=O)NC(c1ccc(F)cc1)c1cnc(N2CCNCC2)nc1]

To a cooled (0-5° C.) solution of tert-butyl 4-{5-[{[(S)-tert-butylsulfinyl]amino}(4-fluorophenyl)methyl]pyrimidin-2-yl}piperazine-1-carboxylate (0.100 g, 0.203 mmol, as prepared in the previous step) in DCM (0.6 ml) was added TFA (0.313 mL, 4.07 mmol). After 2 h, the mixture was concentrated under reduced pressure. The residue was dissolved in DCM and washed with sat. aq. NaHCO₃. The aqueous layer was back extracted twice with DCM and the combined organics washed with water, dried over anhydrous Na₂SO₄, and filtered. The filtrate was concentrated under reduced pressure to afford 78 mg (98%) of N-[(4-fluorophenyl)(2-piperazin-1-ylpyrimidin-5-yl)methyl]-2-methylpropane-2-sulfinamide as a gummy solid that was used without further purification. ¹H NMR (400 MHZ, CDCl₃) δ (ppm) 8.29 (s, 2H), 7.29-7.40 (m, 2H), 6.97-7.07 (m, 2H), 5.46 (d, J=2.57 Hz, 1H), 3.73-3.82 (m, 4H), 3.71 (d, J=2.57 Hz, 1H), 2.78-2.96 (m, 4H), 1.21-1.28 (m, 9H). MS (ESI) m/z [M+H]⁺ calcd. for C₁₉H₂₇FN₅OS, 392.2; found, 392.2; HPLC purity: 210 nm: 91.9%; 254 nm: 95.4%.

### Step 4. Preparation of N-[(4-fluorophenyl)(2-{4-[6-(1-methyl-1H-pyrazol-4-yl)pyrazolo[1,5-a]pyridin-3-yl]piperazin-1-yl}pyrimidin-5-yl)methyl]-2-methylpropane-2-sulfinamide

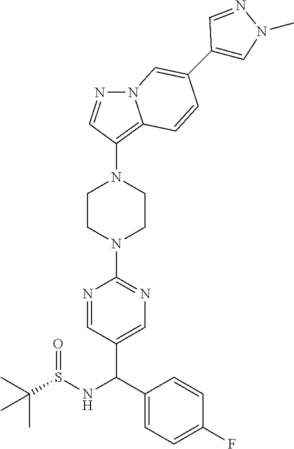 [SMILES: Cn1cc(-c2ccc3c(N4CCN(c5ncc(C(N[S@@](=O)C(C)(C)C)c6ccc(F)cc6)cn5)CC4)cnn3c2)cn1]

To 3-bromo-6-(1-methyl-1H-pyrazol-4-yl)pyrazolo[1,5-a]pyridine (0.044 g, 0.16 mmol) and N-[(4-fluorophenyl)(2-piperazin-1-ylpyrimidin-5-yl)methyl]-2-methylpropane-2-sulfinamide (0.075 g, 0.19 mmol) was added tBuOH (1.0 mL) and dioxane (0.66 mL). The mixture was sparged with argon for 5 min and NaOtBu (0.023 g, 0.24 mmol) was added. The mixture was sparged with argon 5 min and tBuXPhos Pd G1 (11 mg, 0.02 mmol) was added. The mixture was sparged for 5 min with argon and an additional 2 min with sonication. The reaction was stirred at 55° C. for 9 h at 55° C. in a sealed vial then the temperature was increased to 100° C. for an additional 16 h. The mixture was cooled to rt and DCM and sat. aq. NaHCO₃ were added. The aqueous. Layer was extracted with DCM (3×). The combined organic layers were washed with water, dried over anhydrous Na₂SO₄, and filtered. The filtrate was concentrated under reduced pressure. The residue was purified by silica gel column chromatography eluting with a gradient of 0-10% MeOH in DCM to afford 25 mg (27%) of N-[(4-fluorophenyl)(2-{4-[6-(1-methyl-1H-pyrazol-4-yl)pyrazolo[1,5-a]pyridin-3-yl]piperazin-1-yl}pyrimidin-5-yl)methyl]-2-methylpropane-2-sulfinamide as a solid. ¹H NMR (400 MHZ, CDCl₃) δ (ppm) 8.48 (s, 1H), 8.36 (s, 2H), 7.76 (s, 1H), 7.72 (s, 1H), 7.63 (s, 1H), 7.56 (d, J=9.05 Hz, 1H), 7.33-7.42 (m, 2H), 7.16 (dd, J=9.17, 1.34 Hz, 1H), 7.02-7.12 (m, 2H), 5.52 (d, J=2.45 Hz, 1H), 4.02-4.09 (m, 4H), 3.96-4.02 (m, 3H), 3.69 (d, J=2.45 Hz, 1H), 3.07-3.19 (m, 4H), 1.27-1.33 (m, 9H). MS (ESI) m/z [M+H]⁺ calcd. for C₃₀H₃₅FN₉OS, 588.3; found, 588.5; HPLC purity: 210 nm: 100.0%; 254 nm: 100.0%.

### Step 5. Preparation of 1-(4-fluorophenyl)-1-(2-{4-[6-(1-methyl-1H-pyrazol-4-yl)pyrazolo[1,5-a]pyridin-3-yl]piperazin-1-yl}pyrimidin-5-yl)methanamine (Compound 246)

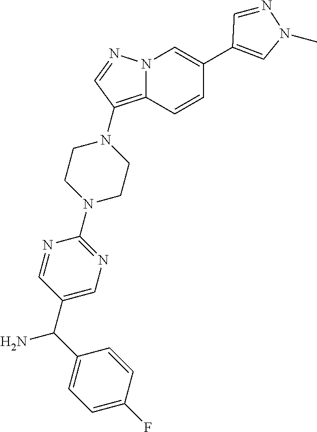 [SMILES: Cn1cc(-c2ccc3c(N4CCN(c5ncc(C(N)c6ccc(F)cc6)cn5)CC4)cnn3c2)cn1]

To a solution of N-[(4-fluorophenyl)(2-{4-[6-(1-methyl-1H-pyrazol-4-yl)pyrazolo[1,5-a]pyridin-3-yl]piperazin-1-yl}pyrimidin-5-yl)methyl]-2-methylpropane-2-sulfinamide (0.025 g, 0.04 mmol, as prepared in the previous step) in DCM (1 mL) was added 4M HCl in dioxane (0.064 mL, 0.26 mmol). The solution was stirred at 40° C. for 3 h, then the mixture was cooled to rt and concentrated under reduced pressure. The resulting solids were triturated with MTBE, filtered, and washed with additional MTBE. The solids were suspended in DCM and washed with sat. aq. NaHCO₃ and water, then dried over anhydrous Na₂SO₄, and filtered. The filtrate was concentrated under reduced pressure. The crude product was stirred with 4.0 eq of MP carbonate resin (1.0 g) in 10 mL of MeOH for 1 h. The resin was removed by filtration through Celite-545R and the filter cake was rinsed with 10 mL of MeOH. The filtrate was evaporated to provide crude product, which was purified by Prep-TLC to give 7 mg (40%) of 1-(4-fluorophenyl)-1-(2-{4-[6-(1-methyl-1H-pyrazol-4-yl)pyrazolo[1,5-a]pyridin-3-yl]piperazin-1-yl}pyrimidin-5-yl)methanamine (Compound 246) as a yellow foam. ¹H NMR (400 MHZ, CDCl₃) δ (ppm) 8.48 (s, 1H), 8.35 (s, 2H), 7.74 (d, J=13.82 Hz, 2H), 7.63 (s, 1H), 7.57 (d, J=9.29 Hz, 1H), 7.39 (dd, J=8.56, 5.38 Hz, 2H), 7.28 (s, 1H), 7.16 (dd, J=9.17, 1.34 Hz, 1H), 7.05 (t, J=8.62 Hz, 2H), 5.13 (s, 1H), 3.93-4.07 (m, 7H), 3.09-3.17 (m, 4H). MS (ESI) m/z [M+H]⁺ calcd. for C₂₆H₂₇FN₉: 483.5; found: 484.3; HPLC purity: 210 nm; 100.0%; 254 nm: 100.0%.

### Example 185: Synthesis of Exemplary Compound 247

### 5-benzyl-2-(4-(6-(1-methyl-1H-pyrazol-4-yl)pyrazolo[1,5-a]pyridin-3-yl)piperazin-1-yl)oxazole (Compound 247)

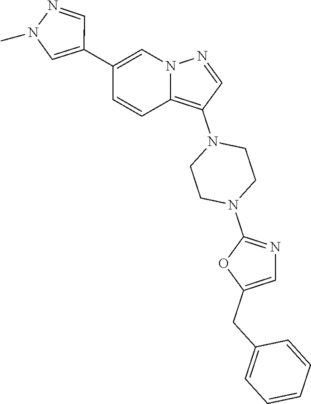 [SMILES: Cn1cc(-c2ccc3c(N4CCN(c5ncc(Cc6ccccc6)o5)CC4)cnn3c2)cn1]

### Step 1. Preparation of tert-butyl 4-(5-(hydroxy(phenyl)methyl)oxazol-2-yl)piperazine-1-carboxylate

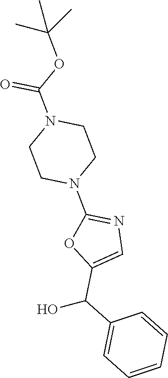 [SMILES: CC(C)(C)OC(=O)N1CCN(c2ncc(C(O)c3ccccc3)o2)CC1]

A solution of tert-butyl 4-(5-benzoyl-1,3-oxazol-2-yl)piperazine-1-carboxylate (Compound S79) (1.00 g, 2.80 mmol) in MeOH (20 mL) was cooled to 0° C. then NaBH₄ (212 mg, 5.60 mmol) was added. The reaction was warmed to rt and stirred for 2 h. The reaction was quenched with 0.1M aq. HCl (40 mL) then the mixture was extracted with EtOAc (3×40 mL). The combined organic layers were washed with sat. aq. NaHCO₃ (4 mL), dried with anhydrous Na₂SO₄, and filtered. The filtrate was concentrated under reduced pressure then the residue was treated with hexane and concentrated under reduced pressure to afford 1.02 g (101%) of tert-butyl 4-(5-(hydroxy(phenyl)methyl)oxazol-2-yl)piperazine-1-carboxylate as a white solid. ¹H NMR (400 MHZ, CDCl₃) δ (ppm) 7.49-7.43 (m, 2H), 7.42-7.31 (m, 3H), 6.45 (s, 1H), 5.76 (d, J=2.4 Hz, 1H), 3.57-3.37 (m, 8H), 2.46 (d, J=4.0 Hz, 1H) 1.48 (s, 9H).

### Step 2. Preparation of tert-butyl 4-(5-benzyloxazol-2-yl)piperazine-1-carboxylate

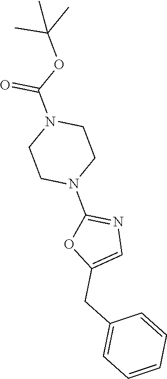 [SMILES: CC(C)(C)OC(=O)N1CCN(c2ncc(Cc3ccccc3)o2)CC1]

To a solution of tert-butyl 4-{5-[hydroxy(phenyl)methyl]-1,3-oxazol-2-yl}piperazine-1-carboxylate (1.01 g, 2.81 mmol, as prepared in the previous step) in DCM (9 mL) was added Et₃SiH (8.3 mL, 52 mmol) and TFA (8.3 mL, 110 mmol). The reaction was stirred at rt overnight. The reaction was concentrated under reduced pressure then toluene was added, and the mixture was concentrated under reduced pressure. This was repeated six times. The residue was dissolved in DCM (20 mL) then 1M NaOH solution (10 mL) and a solution of Boc₂O (0.613 g, 2.81 mmol) in DCM (10 mL) were added. The mixture was stirred at rt overnight. The layers were separated then the aqueous layer was extracted with DCM (3×). The combined organic layers were washed with brine, dried over anhydrous Na₂SO₄, and filtered. The filtrate was concentrated under reduced pressure. The residue was purified by silica gel column chromatography eluting with 0-70% EtOAc/hexane to afford 691.3 mg (72%) of tert-butyl 4-(5-benzyloxazol-2-yl)piperazine-1-carboxylate as a white solid. ¹H NMR (400 MHZ, CDCl₃) δ (ppm) 7.36-7.29 (m, 2H), 7.29-7.21 (m, 3H), 6.46 (s, 1H), 3.88 (s, 2H), 3.55-3.46 (m, 4H), 3.45-3.37 (m, 4H), 1.48 (s, 9H).

### Step 3. Preparation of 5-benzyl-2-(piperazin-1-yl)oxazole

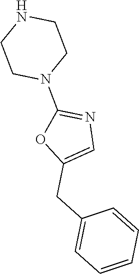 [SMILES: c1ccc(Cc2cnc(N3CCNCC3)o2)cc1]

To a solution of tert-butyl 4-(5-benzyl-1,3-oxazol-2-yl)piperazine-1-carboxylate (0.69 g, 2.0 mmol, as prepared in the previous step) in MeOH (30 mL) was added 1.6 g of AmberChrome50WX4 Ion exchange resin (previously washed with DMF (×3), water (×3), MeOH (×3), DCM (×3), MeOH (×3), and DCM (×3)) then the mixture was warmed to 50° C. with gentle stirring for 1.5 h. The reaction was cooled to rt, and the resin was isolated by filtration. The resin was washed first with DCM (3×10 mL) and MeOH (3×10 mL), then the resin was washed with 3.5 M NH₃ in MeOH (5×10 mL) into a clean flask. The methanolic ammonia filtrate was concentrated under reduced pressure to afford 475.9 mg (97%) of 5-benzyl-2-(piperazin-1-yl)oxazole as a white solid. ¹H NMR (400 MHZ, CDCl₃) δ (ppm) 7.38-7.20 (m, 5H), 6.45 (s, 1H), 3.87 (s, 2H), 3.48-3.36 (m, 4H), 3.00-2.88 (m, 4H).

### Step 4. Preparation of 5-benzyl-2-(4-(6-(1-methyl-1H-pyrazol-4-yl)pyrazolo[1,5-a]pyridin-3-yl)piperazin-1-yl)oxazole (Compound 247)

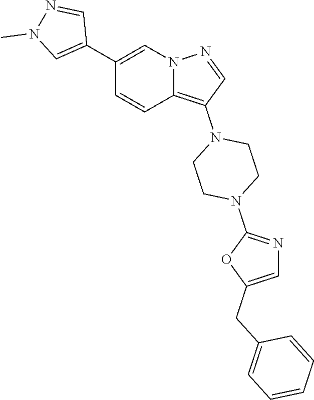 [SMILES: Cn1cc(-c2ccc3c(N4CCN(c5ncc(Cc6ccccc6)o5)CC4)cnn3c2)cn1]

A solution of 3-bromo-6-(1-methyl-1H-pyrazol-4-yl)pyrazolo[1,5-a]pyridine (Compound S9) (243 mg, 0.877 mmol) and 5-benzyl-2-(piperazin-1-yl)oxazole (226 mg, 0.930 mmol, as prepared in the previous step) in tBuOH (2.18 mL) and dioxane (0.548 mL) was sparged with argon for 10 min then NaOtBu (101 mg, 1.05 mmol) was added and the mixture was sparged for 5 min with argon. tBuXPhos Pd G1 (90.3 mg, 0.132 mmol) was added, and the mixture was sparged for 4 min with argon. The reaction was stirred at 55° C. under argon for 19.5 h. The reaction was cooled to rt, diluted with DCM (10 mL) and filtered through a pad of celite. The celite pad was rinsed with DCM (40 mL) then the filtrate was washed with sat. aq. NaHCO₃ (15 mL) and the layers were separated. The aqueous layer was extracted with DCM (3×15 mL) then the combined organic layers were washed with brine (15 mL), dried over anhydrous Na₂SO₄, and filtered. The filtrate was concentrated under reduced pressure. The residue was purified by silica gel flash chromatography eluting with 0-100% acetone/DCM. The crude product was purified by reverse phase chromatography to afford 112 mg (29%) of 5-benzyl-2-(4-(6-(1-methyl-1H-pyrazol-4-yl)pyrazolo[1,5-a]pyridin-3-yl)piperazin-1-yl)oxazole (Compound 247) as a yellow solid. ¹H NMR (400 MHZ, CDCl₃) δ (ppm) 8.47 (s, 1H), 7.75 (s, 1H), 7.72 (s, 1H), 7.61 (s, 1H), 7.52 (dd, J=9.2, 0.7 Hz, 1H), 7.37-7.30 (m, 2H), 7.30-7.23 (m, 3H), 7.15 (dd, J=9.2, 1.5 Hz, 1H), 6.49 (s, 1H), 3.98 (s, 3H), 3.90 (s, 2H), 3.70-3.62 (m, 4H), 3.17-3.09 (m, 4H). MS (ESI) m/z [M+H]⁺ calcd. for C₂₅H₂₅N₇O: 440.2, Found: 440.1; HPLC purity: 210 nm: 99.7%; 254 nm: 99.6%.

### Example 186: Synthesis of Exemplary Compound 248

### (2-(4-(6-(1-methyl-1H-pyrazol-4-yl)pyrazolo[1,5-a]pyridin-3-yl)piperazin-1-yl) oxazol-5-yl)(phenyl)methanol (Compound 248)

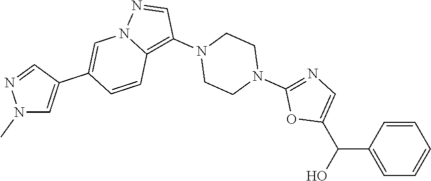 [SMILES: Cn1cc(-c2ccc3c(N4CCN(c5ncc(C(O)c6ccccc6)o5)CC4)cnn3c2)cn1]

### Step 1. Preparation of (9H-fluoren-9-yl)methyl 4-(5-benzoyloxazol-2-yl)piperazine-1-carboxylate

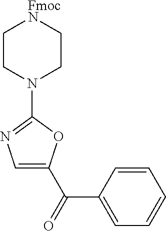

A solution of tert-butyl 4-(5-benzoyl-1,3-oxazol-2-yl)piperazine-1-carboxylate (Compound S79) (5.00 g, 14.0 mmol) in DCM (125 mL) was cooled to 0° C. TFA (32.3 mL, 4.20 mmol) was added dropwise over 10 min and stirred at 0° C. for 3.5 h. The mixture was concentrated under reduced pressure then the residue was treated with toluene and concentrated under reduced pressure. This was repeated seven times. To the residue was added DCM (60.0 mL) then sat. aq. NaHCO₃ solution was slowly added until foaming ceased, and the pH of the mixture was 7-8. A solution of Fmoc-OSu (4.96 g, 14.7 mmol) in DCM (40 mL) was added and the mixture was stirred at rt overnight. The phases were separated, and the aqueous layer was extracted with DCM (2×). The combined organic layers were dried over anhydrous Na₂SO₄, and filtered. The filtrate was concentrated under reduced pressure and the residue was purified by silica gel flash chromatography eluting with 0-20% acetone/DCM to afford 6.59 g (98%) of (9H-fluoren-9-yl)methyl 4-(5-benzoyloxazol-2-yl)piperazine-1-carboxylate as a white foam. ¹H NMR (400 MHZ, CDCl₃) δ (ppm) 7.88-7.76 (m, 4H), 7.63-7.47 (m, 6H), 7.46-7.39 (m, 2H), 7.38-7.31 (m, 2H), 4.58 (d, J=6.0 Hz, 2H), 4.27 (t, J=6.1 Hz, 1H), 3.80-3.33 (m, 8H). MS (ESI) m/z [M+H]⁺ calcd. for C₂₉H₂₅N₃O₄: 480.2, Found: 480.1; HPLC purity: 210 nm: 100.0%; 254 nm: 100.0%.

### Step 2. Preparation of (9H-fluoren-9-yl)methyl 4-(5-(hydroxy(phenyl)methyl)oxazol-2-yl)piperazine-1-carboxylate

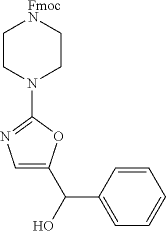

A solution of 9H-fluoren-9-ylmethyl 4-(5-benzoyl-1,3-oxazol-2-yl)piperazine-1-carboxylate (6.53 g, 13.6 mmol, as prepared in the previous step) in THF (90 mL) and EtOH (30 mL) was cooled to 0° C. then NaBH₄ (1.03 g, 27.2 mmol) was added. The reaction was warmed to rt and stirred for 2.5 h then quenched with 0.1 HCl (450 mL). The mixture was extracted with DCM (3×250 mL and 2×200 mL). The organic extracts were combined, washed with sat. aq. NaHCO₃ (200 mL), dried over anhydrous Na₂SO₄, and filtered. The filtrate was concentrated under reduced pressure, then the residue was treated with hexanes and concentrated under reduced pressure. This was repeated twice more to afford 6.37 g (97%) of (9H-fluoren-9-yl)methyl 4-(5-(hydroxy(phenyl)methyl)oxazol-2-yl)piperazine-1-carboxylate as a white foam. ¹H NMR (400 MHZ, CDCl₃) δ (ppm) 7.77 (d, J=7.58 Hz, 2H), 7.57 (d, J=7.34 Hz, 2H), 7.49-7.28 (m, 9H), 6.47 (s, 1H), 5.77 (d, J=4.0 Hz, 1H), 4.51 (d, J=6.5 Hz, 2H), 4.26 (t, J=6.42 Hz, 1H), 3.66-3.26 (m, 8H), 2.33 (d, J=4.4 Hz, 1H).

### Step 3. Preparation of (9H-fluoren-9-yl)methyl 4-(5-(((tert-butyldimethylsilyl)oxy)(phenyl)methyl)oxazol-2-yl)piperazine-1-carboxylate

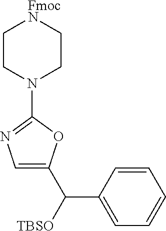

To a solution of 9H-luoren-9-ylmethyl 4-{5-[hydroxy(phenyl)methyl]-1,3-oxazol-2-yl}piperazine-1-carboxylate (6.35 g, 13.2 mmol, as prepared in the previous step) in DMF (40 mL) were added 1H-imidazole (8.977 g, 131.9 mmol) and TBSCl (9.938 g, 65.93 mmol). The reaction was stirred at rt overnight. The mixture was concentrated under reduced pressure then the residue was partitioned between EtOAc (160 mL) and water (160 mL). The aqueous layer was extracted with EtOAc (2×160 mL). The organic layers were combined, dried with anhydrous Na₂SO₄, and filtered. The filtrate was concentrated under reduced pressure. The residue was purified by flash chromatography eluting with 0-80% EtOAc/hexane to afford 4.05 g (52%) of (9H-fluoren-9-yl)methyl 4-(5-(((tert-butyldimethylsilyl)oxy)(phenyl)methyl)oxazol-2-yl)piperazine-1-carboxylate as a white foam. ¹H NMR (400 MHZ, CDCl₃) δ ppm 7.77 (d, J=7.6 Hz, 2H), 7.57 (d, J=7.2 Hz, 2H), 7.46-7.28 (m, 9H), 6.35 (s, 1H), 5.72 (s, 1H), 4.51 (d, J=6.4 Hz, 2H), 4.26 (t, J=6.4 Hz, 1H), 3.61-3.24 (m, 8H), 0.93 (s, 9H), 0.08 (s, 3H), 0.07 (s, 3H).

### Step 4. Preparation of 5-(((tert-butyldimethylsilyl)oxy) (phenyl)methyl)-2-(piperazin-1-yl)oxazole

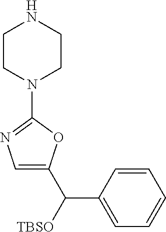 [SMILES: CC(C)(C)[Si](C)(C)OC(c1ccccc1)c1cnc(N2CCNCC2)o1]

To a solution of 9H-fluoren-9-ylmethyl 4-{5-[{[tert-butyl(dimethyl)silyl]oxy}(phenyl)methyl]-1,3-oxazol-2-yl}piperazine-1-carboxylate (1.00 g, 1.68 mmol, as prepared in the previous step) in DCM (20 mL) cooled to 0° C. was added Et₂NH (7.81 mL, 75.5 mmol) then the reaction was warmed to rt and stirred for 6.5 h. The mixture was concentrated under reduced pressure. The residue was suspended in DCM (10 mL), filtered, and the filter pad was washed with DCM (5 mL). The filtrate was purified by silica gel chromatography eluting with 0-100% of DCM: 7M NH₃ in MeOH (9:1) affording 475 mg (74%) of 5-(((tert-butyldimethylsilyl)oxy) (phenyl)methyl)-2-(piperazin-1-yl)oxazole as a colorless oil. ¹H NMR (400 MHZ, CDCl₃) δ (ppm) 7.44-7.39 (m, 2H), 7.38-7.28 (m, 3H), 6.31 (d, J=0.8 Hz, 1H), 5.71 (s, 1H), 3.44-3.40 (m, 4H), 2.94-2.90 (m, 4H), 0.92 (s, 9H), 0.07 (s, 3H), 0.06 (s, 3H). MS (ESI) m/z [M+H]⁺ calcd. for C₂₀H₃₁N₃O₂Si: 374.2, Found: 374.0; HPLC purity: 210 nm: 97.4%; 254 nm: 95.3%.

### Step 5. Preparation of 5-(((tert-butyldimethylsilyl)oxy) (phenyl)methyl)-2-(4-(6-(1-methyl-1H-pyrazol-4-yl)pyrazolo[1,5-a]pyridin-3-yl)piperazin-1-yl)oxazole

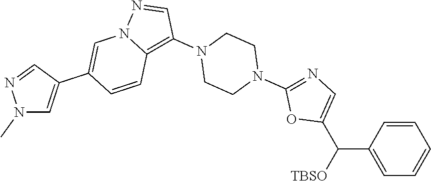 [SMILES: Cn1cc(-c2ccc3c(N4CCN(c5ncc(C(O[Si](C)(C)C(C)(C)C)c6ccccc6)o5)CC4)cnn3c2)cn1]

A solution of 3-bromo-6-(1-methyl-1H-pyrazol-4-yl)pyrazolo[1,5-a]pyridine (Compound S9) (290 mg, 1.05 mmol) and 5-(((tert-butyldimethylsilyl)oxy) (phenyl)methyl)-2-(piperazin-1-yl)oxazole (470 mg, 1.26 mmol, as prepared in the previous step) in tBuOH (2.90 mL) and dioxane (1.46 mL) was sparged with argon for 10 min then KOtBu (176.5 mg, 1.573 mmol) was added and the mixture was sparged for 5 min with argon. tBuXPhos Pd G1 (108 mg, 0.157 mmol) was added, and the mixture was sparged for 4 min with argon. The mixture was heated at 55° C. under an argon atmosphere and stirred for 1 h. The reaction was cooled to rt, diluted with DCM (10 mL) and filtered through a pad of celite. The celite pad was rinsed with DCM (4×10 mL) then the combined filtrate was washed with sat. aq. NaHCO₃ (20 mL) and the layers were separated. The aqueous layer was extracted with DCM (3×15 mL) then the combined organic layers were washed with brine (15 mL), dried over anhydrous Na₂SO₄, and filtered. The filtrate was concentrated under reduced pressure. The residue was purified by flash chromatography eluting with 0-10% EtOAc/IPA, and then 30% IPA/DCM to afford 315 mg (53%) of 5-(((tert-butyldimethylsilyl)oxy) (phenyl)methyl)-2-(4-(6-(1-methyl-1H-pyrazol-4-yl)pyrazolo[1,5-a]-608-yridine-3-yl)piperazin-1-yl)oxazole as a tan foam. ¹H NMR (400 MHz, CDCl₃) δ (ppm) 8.47 (s, 1H), 7.75 (s, 1H), 7.72 (s, 1H), 7.62 (s, 1H), 7.52 (d, J=9.6 Hz, 1H), 7.46-7.41 (m, 2H), 7.36 (t, J=7.2 Hz, 2H), 7.33-7.28 (m, 1H), 7.15 (dd, J=9.2, 1.6 Hz, 1H), 6.35 (d, J=0.4 Hz, 1H), 5.74 (s, 1H), 3.98 (s, 3H), 3.70-3.64 (m, 4H), 3.16-3.10 (m, 4H), 0.93 (s, 9H), 0.09 (s, 3H), 0.08 (s, 3H). MS (ESI) m/z [M+H]⁺ calcd. For C₃₁H₃₉N₇O₂Si: 570.3, Found: 570.2; HPLC purity: 210 nm: 92.3%; 254 nm: 92.3%.

### Step 6. Preparation of (2-(4-(6-(1-methyl-1H-pyrazol-4-yl)pyrazolo[1,5-a]-609-yridine-3-yl)piperazin-1-yl)oxazol-5-yl)(phenyl)methanol (Compound 248)

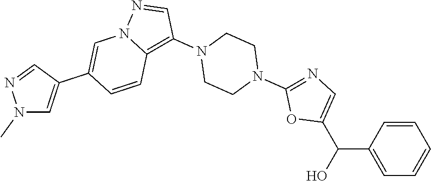 [SMILES: Cn1cc(-c2ccc3c(N4CCN(c5ncc(C(O)c6ccccc6)o5)CC4)cnn3c2)cn1]

To a solution of 5-(((tert-butyldimethylsilyl)oxy) (phenyl)methyl)-2-(4-(6-(1-methyl-1H-pyrazol-4-yl)pyrazolo[1,5-a]-609-yridine-3-yl)piperazin-1-yl)oxazole (150 mg, 0.26 mmol, as prepared in the previous step) in THF (8 mL) was added 1M TBAF in THF (0.42 mL, 0.42 mmol) and the reaction was stirred at rt for 5 min. The mixture was diluted with EtOAc, washed with brine, dried over anhydrous Na₂SO₄, and filtered. The filtrate was concentrated under reduced pressure then the residue was purified by flash eluting with 0-100% of 10% MeOH in DCM/DCM. The resulting product was dissolved in DCM (15 mL), washed with water (5×5 mL), dried over anhydrous Na₂SO₄, and filtered. The filtrate was concentrated under reduced pressure affording 83.1 mg (69%) of (2-(4-(6-(1-methyl-1H-pyrazol-4-yl)pyrazolo[1,5-a]-609-yridine-3-yl)piperazin-1-yl)oxazol-5-yl)(phenyl)methanol (Compound 248) as a light brown foam. ¹H NMR (400 MHz, CDCl₃) δ (ppm) 8.47 (s, 1H), 7.75 (s, 1H), 7.72 (s, 1H), 7.61 (s, 1H), 7.52 (d, J=9.2 Hz, 1H), 7.48 (d, J=7.2 Hz, 2H), 7.43-7.32 (m, 3H), 7.16 (dd, J=9.2, 1.6 Hz, 1H), 6.49 (d, J=0.6 Hz, 1H), 5.79 (br s, 1H), 3.98 (s, 3H), 3.72-3.68 (m, 4H), 3.15-3.11 (m, 4H), 2.43 (d, J=2.8 Hz, 1H). MS (ESI) m/z [M+H]⁺ calcd. for C₂₅H₂₅N₇O₂: 456.2, Found: 456.2; HPLC purity: 210 nm: 100.0%; 254 nm: 100.0%.

### Example 187: Synthesis of Exemplary Compound 249

### 3-[4-(5-benzylpyridin-2-yl)piperazin-1-yl]-6-(1-methyl-1H-pyrazol-4-yl)pyrazolo[1,5a]pyridine (Compound 249)

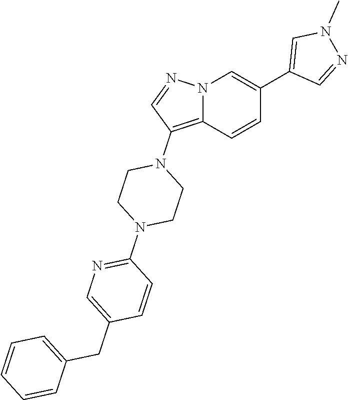 [SMILES: Cn1cc(-c2ccc3c(N4CCN(c5ccc(Cc6ccccc6)cn5)CC4)cnn3c2)cn1]

### Step 1. Preparation of 5-benzyl-2-chloropyridine

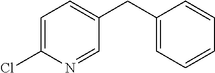 [SMILES: Clc1ccc(Cc2ccccc2)cn1]

A mixture of (6-chloro-3-pyridinyl) boronic acid (1.00 g, 6.35 mmol), Na₂CO₃ (2.02 g, 19.1 mmol), and (Ph₃P)₂PdCl₂ (223 mg, 0.32 mmol) in dioxane (5.0 mL) and water (2.0 mL) was sparged with nitrogen for 15 min. The mixture was treated with benzyl bromide (0.831 mL, 6.99 mmol) and the reaction was heated at 100° C. for 2 h. The reaction mixture was cooled to rt, diluted with water (50 mL), and extracted with EtOAc (2×50 mL). The combined organic layers were washed with brine, dried over Na₂SO₄, and concentrated to yield brown oil that was purified by flash chromatography to yield 743 mg (57%) of 5-benzyl-2-chloropyridine as a yellow solid. ¹H NMR (400 MHz, CDCl₃) δ (ppm) 8.28 (d, J=2.2 Hz, 1H), 7.43 (dd, J=8.2, 2.6, 1H), 7.32 (m, 2H), 7.25 (m, 2H), 7.16 (d, J=7.1 Hz 2H), 3.97 (s, 2H). MS (ESI) m/z [M+H]⁺ calcd. for C₁₂H₁₁ClN: 204.1; found, 203.9; HPLC purity 210 nm: 100.0%; 254 nm: 100.0%.

### Step 2. Preparation of tert-butyl 4-(5-benzylpyridin-2-yl)piperazine-1-carboxylate

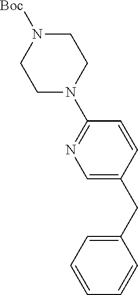 [SMILES: CC(C)(C)OC(=O)N1CCN(c2ccc(Cc3ccccc3)cn2)CC1]

Toluene (4.0 mL) was added to 5-benzyl-2-chloropyridine (200 mg, 0.98 mmol, as prepared in the previous step) and tert-butyl-1-piperazinecarboxylate (229 mg, 1.23 mmol). The mixture was sparged with nitrogen gas for 20 min. Pd₂(dba)₃ (44 mg, 0.05 mmol), BINAP (62 mg, 0.10 mmol), and KOtBu (331 mg, 2.95 mmol) were added and the mixture was sparged with nitrogen for 5 min. The reaction was heated at 100° C. for 2 h under nitrogen. The reaction was cooled to rt and partitioned between water and EtOAc. The organic layer was washed with brine, dried with Na₂SO₄, and concentrated under vacuum. The residue was purified by silica gel column chromatography to afford 236 mg (68%) of tert-butyl 4-(5-benzylpyridin-2-yl)piperazine-1-carboxylate as a solid. ¹H NMR (400 MHZ, DMSO-d₆) δ (ppm) 8.04 (d, J=2.1, 1H) 7.39 (dd, J=8.8, 2.4, 1H), 7.27 (m, 2H), 7.18 (m, 3H), 6.77 (d, J=8.8, 1H), 3.80 (s, 2H), 3.40 (s, 8H), 1.41 (s, 9H). MS (ESI) m/z [M+H]⁺ calcd. for C₂₁H₂₈N₃O₂: 354.2; found, 354.1; HPLC purity: 210 nm: 98.5%; 254 nm 100.0%.

### Step 3. Preparation of 1-(5-benzyl-2-pyridin-2-yl)piperazine

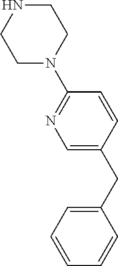 [SMILES: c1ccc(Cc2ccc(N3CCNCC3)nc2)cc1]

A solution of tert-butyl 4-(5-benzylpyridin-2-yl)piperazine-1-carboxylate (230 mg, 0.65 mmol, as prepared in the previous step) in MeOH (9.5 mL) was treated with Dowex® 50WX4-200 ion exchange resin (759 mg, washed and dried) at 50° C. After an hour the reaction was filtered, and the resin was washed alternately with MeOH and THF. The product was extracted from the resin by washing the resin with 3.5M NH₃ in MeOH and THF. The solvent was removed under reduced pressure to afford 159 mg (96%) of 1-(5-benzyl-2-pyridin-2-yl)piperazine as a solid. ¹H NMR (400 MHZ, CDCl₃) δ (ppm) 8.10 (s, 1H), 7.29 (m, 3H), 7.19 (m, 3H), 6.60 (d, J=8.7, 1H), 3.86 (s, 2H), 3.48 (m, 4H), 3.00 (dd, J=5.9, 4.2, 4H). MS (ESI) m/z [M+H]⁺ calcd. for C₁₆H₂₀N₃: 254.2; found, 254.1; HPLC purity: 210 nm: 96.1%; 254 nm: 100.0%.

### Step 4. 3-[4-(5-benzylpyridin-2-yl)piperazin-1-yl]-6-(1-methyl-1H-pyrazol-4-yl)pyrazolo[1,5a]pyridine (Compound 249)

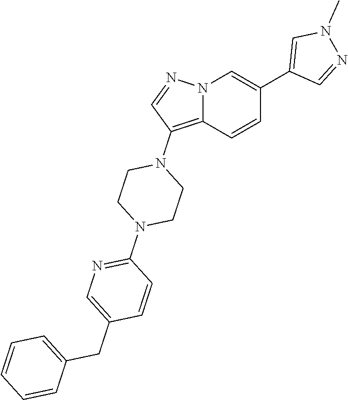 [SMILES: Cn1cc(-c2ccc3c(N4CCN(c5ccc(Cc6ccccc6)cn5)CC4)cnn3c2)cn1]

A solution of 3-bromo-6-(1-methyl-1H-pyrazol-4-yl)pyrazolo[1,5-a]pyridine (Compound S9) (145 mg, 0.523 mmol), 1-(5-benzyl-2-pyridin-2-yl)piperazine (159 mg, 0.628 mmol, as prepared in the previous step) in tBuOH (2.0 mL) and dioxane (1.0 mL) was sparged for 15 min with argon. The solution was treated with tBuXPhos Pd G1 (72 mg, 0.105 mmol) and NaOtBu (75 mg, 0.784 mmol) and the reaction mixture was sparged with argon for 3 min. The reaction mixture was heated at 52° C. for 4 h then cooled to rt and concentrated to a brown solid. The residue was purified by flash chromatography to yield 67 mg (34%) of 3-[4-(5-benzylpyridin-2-yl)piperazin-1-yl]-6-(1-methyl-1H-pyrazol-4-yl)pyrazolo[1,5^{a}]pyridine (Compound 249) as a yellow solid: ¹H NMR (400 MHZ, DMSO-d₆) δ (ppm) 8.82 (s, 1H), 8.22 (s, 1H), 8.06 (d, J=2.2 Hz, 1H), 7.97 (s, 1H), 7.78 (s, 1H), 7.67 (d, J=8.9 Hz, 1H), 7.40 (dd, J=8.7, 2.2 Hz, 1H), 7.30 (m, 3H), 7.19 (m, 3H), 6.83 (d, J=8.70 Hz, 1H), 3.87 (s, 3H), 3.82 (s, 2H), 3.62 (m, 4H), 3.07 (m, 4H). MS (ESI) m/z [M+H]⁺ calcd. for C₂₇H₂₈N₇: 450.2; found, 450.2; HPLC purity: 210 nm: 100.0%; 254 nm: 100.0%.

### Example 188: Synthesis of Exemplary Compound 250

### 6-(1-methyl-1H-pyrazol-4-yl)-3-{4-[5-(pyridine-2-ylmethyl)pyrimidin-2-yl]piperazin-1-yl}pyrazolo[1,5-a]pyridine (Compound 250)

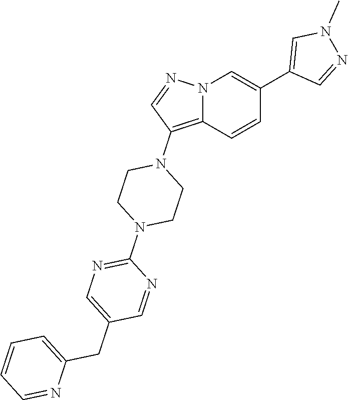 [SMILES: Cn1cc(-c2ccc3c(N4CCN(c5ncc(Cc6ccccn6)cn5)CC4)cnn3c2)cn1]

A mixture of 6-(1-methyl-1H-pyrazol-4-yl)-3-piperazin-1-pyrazolo[1,5-a]pyridine hydrochloride (Compound S19) (92 mg, 0.29 mmol), 2-chloro-5-(pyridine-2-ylmethyl)pyrimidine (Compound S80) (70 mg, 0.34 mmol), K₂CO₃ (123 mg, 0.892 mmol), and NMP (3.7 mL) was sparged for 1 min with argon. The reaction was heated at 110° C. for 2 h then cooled to rt and partitioned between water and DCM. The organic layer was washed with brine, dried over anhydrous Na₂SO₄, and filtered. The Filtrate was concentrated under reduced pressure. The residue was purified by silica gel column chromatography (IPA/DCM) then further purified by C₁₈ reverse-phase silica gel flash chromatography (ACN/water) to yield 30 mg (23%) of 6-(1-methyl-1H-pyrazol-4-yl)-3-{4-[5-(pyridine-2-ylmethyl)pyrimidin-2-yl]piperazin-1-yl}pyrazolo[1,5-a]pyridine (Compound 250) as a solid. ¹H NMR (400 MHZ, DMSO-d₆) δ (ppm) 8.81 (s, 1H), 8.48 (m, 1H), 8.34 (s, 2H), 8.22 (s, 1H), 7.97 (s, 1H), 7.77 (s, 1H), 7.71 (m, 2H), 7.32 (m, 2H), 7.22 (dd, J=6.9, 5.3 Hz, 1H), 3.94 (s, 2H), 3.89 (m, 4H), 3.87 (s, 3H), 3.04 (m, 4H). MS (ESI) m/z [M+H]⁺ calcd. for C₂₅H₂₆N₉: 452.2; found, 452.3; HPLC purity: 210 nm: 100.0%; 254 nm: 100.0%.

### Example 189: Synthesis of Exemplary Compound 251

### 6-(1-methyl-1H-pyrazol-4-yl)-3-{4-[5-(pyridin-3-ylmethyl)pyrimidin-2-yl]piperazin-1-yl}pyrazolo[1,5-a]pyridine (Compound 251)

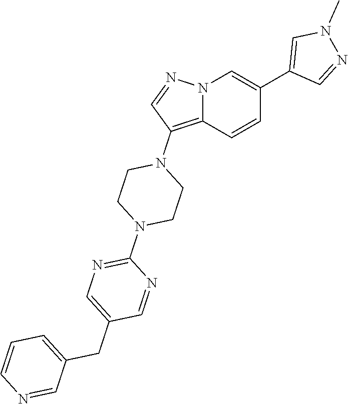 [SMILES: Cn1cc(-c2ccc3c(N4CCN(c5ncc(Cc6cccnc6)cn5)CC4)cnn3c2)cn1]

A mixture of 6-(1-methyl-1H-pyrazol-4-yl)-3-piperazin-1-ylpyrazolo[1,5-a]pyridine hydrochloride (Compound S19) (155 mg, 0.486 mmol), 2-chloro-5-(pyridin-3-ylmethyl)pyrimidine (Compound S81) (105 mg, 0.510 mmol), K₂CO₃ (336 mg, 2.43 mmol), and DMF (3.1 mL) was sparged for 20 min with nitrogen, then 10 min with argon. The reaction mixture was heated at 105° C. for 4 h and then cooled to rt. The solvent was removed under reduced pressure then heptane was added and the mixture was concentrated under reduced pressure. The residue was purified by silica gel column chromatography (IPA/DCM) to afford 82 mg (37%) of 6-(1-methyl-1H-pyrazol-4-yl)-3-{4-[5-(pyridin-3-ylmethyl)pyrimidin-2-yl]piperazin-1-yl}pyrazolo[1,5-a]pyridine (Compound 251) as a solid. ¹H NMR (400 MHZ, DMSO-d₆) δ (ppm) 8.81 (s, 1H), 8.52 (d, J=1.8 Hz, 1H), 8.42 (dd, J=4.7, 1.4 Hz, 1H), 8.34 (s, 2H), 8.22 (s, 1H), 7.96 (s, 1H), 7.77 (s, 1H), 7.66 (m, 2H), 7.33 (s, 1H), 7.31 (m, 1H), 3.89 (m, 7H), 3.83 (s, 2H), 3.03 (m, 4H). MS (ESI) m/z [M+H]⁺ calcd. for C₂₅H₂₆N₉: 452.2; found, 452.2; HPLC purity: 210 nm: 98.7%; 254 nm: 99.2%.

### Example 190: Synthesis of Exemplary Compound 252

### N-isopropyl-N-methyl-4-[6-(1-methyl-1H-pyrazol-4-yl)pyrazolo[1,5-a]pyridin-3-yl]piperazine-1-carboxamide (Compound 252)

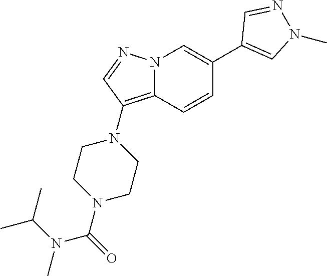 [SMILES: CC(C)N(C)C(=O)N1CCN(c2cnn3cc(-c4cnn(C)c4)ccc23)CC1]

To a solution of 4-nitrophenyl isopropyl(methyl)carbamate (Compound S37) (124 mg, 0.437 mmol) in ACN (4.87 mL) was added 6-(1-methyl-1H-pyrazol-4-yl)-3-piperazin-1-ylpyrazolo[1,5-a]pyridine hydrochloride (Compound S19) (100 mg, 0.314 mmol), then DIPEA (1.3 mL, 7.5 mmol) was added. The reaction was stirred at rt for 2 h then warmed to 35° C., and stirred for 15 h. The reaction was heated to 84° C. for 2 h then cooled to rt and concentrated under reduced pressure. The residue was suspended in DMF (1.00 mL) and Na₂CO₃ (133 mg, 1.25 mmol) was added. The reaction was stirred at 120° C. for 2 h at 120° C., then cooled to rt and stirred for 48 h. The reaction was diluted with water and extracted with EtOAc (2×). The combined organic layers were washed with water (2×) and brine, dried over anhydrous Na₂SO₄, and filtered. The filtrate was concentrated under reduced pressure and the residue was purified by silica gel column chromatography eluting with 10-90% acetone/DCM containing 0.1% Et₃N to afford 30 mg (20%) of N-isopropyl-N-methyl-4-(6-(1-methyl-1H-pyrazol-4-yl)pyrazolo[1,5-a]pyridin-3-yl)piperazine-1-carboxamide (Compound 252) as a beige solid. ¹H NMR (400 MHZ, CDCl₃) δ (ppm) 8.46 (s, 1H), 7.75 (s, 1H), 7.72 (s, 1H), 7.61 (s, 1H) 7.52 (d, J=8.8 Hz, 1H), 7.14 (d, J=8.8, 1H), 4.13 (spt, J=6.8 Hz, 1H), 3.98 (s, 3H), 3.48-3.40 (m, 4H), 3.12-2.99 (m, 4H), 2.75 (s, 3H), 1.17 (d, J=6.4, 6H). MS (ESI) m/z [M+H]⁺ calcd. for C₂₀H₂₇N₇O: 382.2, Found: 382.2; HPLC purity: 210 nm: 97.4%; 254 nm: 98.5%.

### Example 191: Synthesis of Exemplary Compound 253

### 3-[4-(4-methoxybenzyl)piperazin-1-yl]-6-(1-methyl-1H-pyrazol-4-yl)pyrazolo[1,5-a]pyridine (Compound 253)

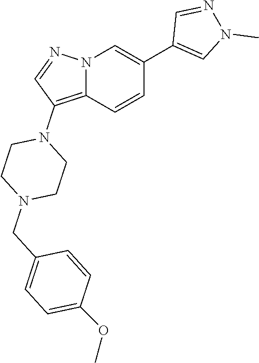 [SMILES: COc1ccc(CN2CCN(c3cnn4cc(-c5cnn(C)c5)ccc34)CC2)cc1]

To a flame dried flask was added 3-bromo-6-(1-methyl-1H-pyrazol-4-yl)pyrazolo[1,5-a]pyridine (Compound S9) (0.200 g, 0.72 mmol) and N-4-methoxybenzylpiperazine (0.179 g, 0.87 mmol) followed by tBuOH (2 mL) and dioxane (1 mL). The mixture was sparged with argon for 5 min and NaOtBu (0.104 g, 1.08 mmol) was added. The mixture was sparged with argon for 5 min and tBuXPhos Pd G1 (50 mg, 0.07 mmol) was added. The mixture was sparged 5 min with argon and an additional 2 min with sonication then heated at 55° C. for 2 h. The mixture was cooled and diluted with DCM and water. The layers were separated. The organic layer was washed with water, dried over anhydrous Na₂SO₄, and filtered. The filtrate was concentrated under reduced pressure. The residue was purified by silica gel column chromatography (eluting with a gradient of 0-10% EtOH in EtOAc containing 0.1% to afford 157 mg (54%) of 3-[4-(4-methoxybenzyl)piperazin-1-yl]-6-(1-methyl-1H-pyrazol-4-yl)pyrazolo[1,5-a]pyridine (Compound 253) as a yellow solid. ¹H NMR (400 MHZ, CDCl₃) δ (ppm) 8.46 (s, 1H), 7.75 (s, 1H), 7.71 (s, 1H), 7.61 (s, 1H), 7.51 (d, J=9.29 Hz, 1H), 7.31 (s, 1H), 7.11 (dd, J=9.17, 1.47 Hz, 1H), 6.90 (d, J=8.56 Hz, 2H), 3.98 (s, 3H), 3.83 (s, 3H), 3.56 (s, 2H), 3.05-3.20 (m, 4H), 2.58-2.78 (m, 4H). MS (ESI) m/z [M+H]⁺ calcd. for C₂₃H₂₇N₆O, 403.2; found, 403.3; HPLC purity: 210 nm: 100.0%; 254 nm: 100.0%.

### Example 192: Synthesis of Exemplary Compound 254

### 3-(4-(5-(4-methoxybenzyl)pyrimidin-2-yl)piperazin-1-yl)-6-(1-methyl-1H-pyrazol-4-yl)pyrazolo[1,5-a]pyridine (Compound 254)

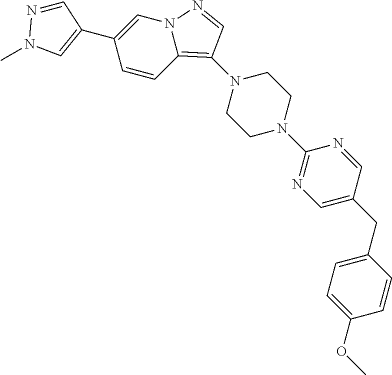 [SMILES: COc1ccc(Cc2cnc(N3CCN(c4cnn5cc(-c6cnn(C)c6)ccc45)CC3)nc2)cc1]

### Step 1. Preparation of tert-butyl 4-(5-(4-methoxybenzyl)pyrimidin-2-yl)piperazine-1-carboxylate

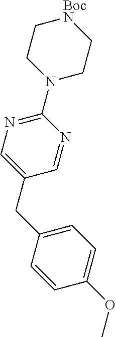 [SMILES: COc1ccc(Cc2cnc(N3CCN(C(=O)OC(C)(C)C)CC3)nc2)cc1]

To a mixture of tert-butyl 4-(5-bromopyrimidin-2-yl)piperazine-1-carboxylate (1.83 g, 5.34 mmol) and Na₂CO₃ (1.71 g, 16.2 mmol) in dioxane (13.8 mL) and H₂O (13.8 mL) was added 2-(4-methoxybenzyl)-4,4,5,5-tetramethyl-1,3,2-dioxaborolane (2.00 g, 8.06 mmol) and the reaction was sparged with argon for 15 min. To the mixture was added Pd(dppf)Cl₂·DCM (0.184 g, 0.225 mmol) with an argon purge. The reaction was stirred at 75° C. overnight. The mixture was cooled to rt and filtered through a plug of Celite. The filter pad was washed with EtOAc then the filtrate was washed with water (100 mL). The aqueous layer was extracted with EtOAc (3×50 mL) then the combined organic extracts were washed with brine (100 mL), dried over anhydrous Na₂SO₄, and filtered. The filtrate was concentrated under reduced pressure and the residue was purified by flash chromatography eluting with 0-50% EtOAc/hexane to afford 0.77 g (38%) of tert-butyl 4-(5-(4-methoxybenzyl)pyrimidin-2-yl)piperazine-1-carboxylate as a pale-yellow solid. ¹H NMR (400 MHZ, CDCl₃) δ (ppm) 8.17 (s, 2H), 7.08 (d, J=8.6 Hz, 2H), 6.84 (d, J=8.6 Hz, 2H), 3.79 (s, 3H), 3.79-3.74 (m, 4H), 3.74 (s, 2H), 3.52-3.48 (m, 4H), 1.50 (s, 9H). MS (ESI) m/z [M+H]⁺ calcd. for C₂₁H₂₈N₄O₃: 385.2, Found: 385.1; HPLC purity: 210 nm: 87.5%; 254 nm: 96.9%.

### Step 2. Preparation of 5-(4-methoxybenzyl)-2-(piperazin-1-yl)pyrimidine

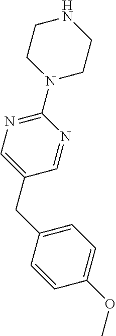 [SMILES: COc1ccc(Cc2cnc(N3CCNCC3)nc2)cc1]

To a solution of tert-butyl 4-(5-(4-methoxybenzyl)pyrimidin-2-yl)piperazine-1-carboxylate (0.57 g, 1.482 mmol, as prepared in the previous step) in MeOH (40 mL) was added 3 g of Dowex50WX4-400 Ion exchange resin (previously washed with DMF (×3), water (×3), MeOH (×3), DCM (×3), MeOH (×3), and DCM (×3)) then the mixture was warmed to 50° C. with gentle stirring for 3.5 h. The reaction was cooled to rt and the resin was isolated by filtration. The resin was washed with MeOH (10 mL), DCM (10 mL), and MeOH (10 mL), then the resin was washed with 3.5M NH₃ in MeOH (8×10 mL) into a clean flask. The methanolic ammonia filtrate was concentrated under reduced pressure affording 289.4 mg (69%) of 5-(4-methoxybenzyl)-2-(piperazin-1-yl)pyrimidine as a white solid. ¹H NMR (400 MHz, CDCl₃) δ (ppm) 8.16 (s, 2H), 7.08 (d, J=8.4 Hz, 2H), 6.83 (d, J=8.4 Hz, 2H), 3.79 (s, 3H), 3.78-3.74 (m, 4H), 3.73 (s, 2H), 2.96-2.90 (m, 4H). MS (ESI) m/z [M+H]⁺ calcd. for C₁₆H₂₀N₄O: 285.2, Found: 285.1; HPLC purity: 210 nm: 100.0%; 254 nm: 100.0%.

### Step 3. Preparation of 3-(4-(5-(4-methoxybenzyl)pyrimidin-2-yl)piperazin-1-yl)-6-(1-methyl-1H-pyrazol-4-yl)pyrazolo[1,5-a]pyridine (Compound 254)

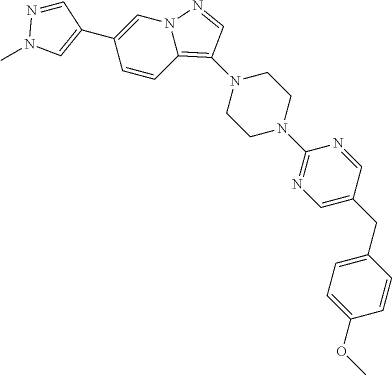 [SMILES: COc1ccc(Cc2cnc(N3CCN(c4cnn5cc(-c6cnn(C)c6)ccc45)CC3)nc2)cc1]

A solution of 3-bromo-6-(1-methyl-1H-pyrazol-4-yl)pyrazolo[1,5-a]pyridine (Compound S9) (138 mg, 0.498 mmol) and 5-(4-methoxybenzyl)-2-(piperazin-1-yl)pyrimidine (150 mg, 0.528 mmol, as prepared in the previous step) in tBuOH (1.24 mL) and dioxane (0.311 mL) was sparged with argon for 15 min then NaOtBu (57.4 mg, 0.597 mmol) was added. The mixture was sparged again for 5 min with argon. tBuXPhos Pd G1 (51.2 mg, 0.0746 mmol) was added, and the mixture was sparged for 5 min with argon. The mixture was stirred at 55° C. under argon for 17 h. The reaction was cooled to rt, filtered through a pad of celite, and the celite pad was rinsed with DCM (50 mL). The filtrate was washed with sat. aq. NaHCO₃ (15 mL) and brine (15 mL), then dried over anhydrous Na₂SO₄ and filtered. The filtrate was concentrated under reduced pressure. The residue was purified by flash chromatography eluting with 0-100% acetone/DCM to afford 112 mg (29%) of 3-(4-(5-(4-methoxybenzyl)pyrimidin-2-yl)piperazin-1-yl)-6-(1-methyl-1H-pyrazol-4-yl)pyrazolo[1,5-a]pyridine (Compound 254) as a tan solid. ¹H NMR (400 MHZ, DMSO-d₆) δ (ppm) 8.81 (s, 1H), 8.28 (s, 2H), 8.22 (s, 1H), 7.97 (s, 1H), 7.77 (s, 1H), 7.68 (d, J=9.2 Hz, 1H), 7.32 (dd, J=9.2, 1.2 Hz, 1H), 7.15 (d, J=8.8 Hz, 2H), 6.86 (d, J=8.4 Hz, 2H), 3.92-3.87 (m, 4H), 3.87 (s, 3H), 3.72 (s, 2H), 3.71 (m, 3H), 3.06-3.00 (m, 4H). MS (ESI) m/z [M+H]⁺ calcd. for C₂₇H₂₈N₈O: 481.2, Found: 481.2; HPLC purity: 210 nm: 96.5%; 254 nm: 98.9%.

### Example 193: Synthesis of Exemplary Compound 255

### 3-[4-(5-benzylpyrimidin-2-yl)piperazin-1-yl]-6-(1H-pyrazol-4-yl)pyrazolo-[1,5-a]pyridine (Compound 255)

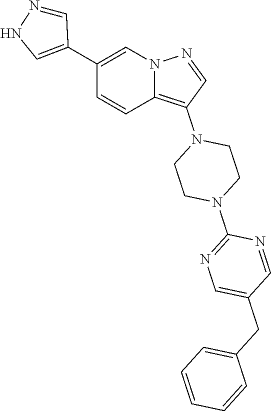 [SMILES: c1ccc(Cc2cnc(N3CCN(c4cnn5cc(-c6cn[nH]c6)ccc45)CC3)nc2)cc1]

### Step 1. Preparation of 6-[1-(1-ethoxylethyl)-1H-pyrazol-4-yl]pyrazolo[1,5-a]pyridine

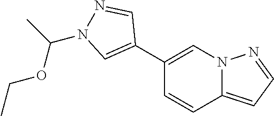 [SMILES: CCOC(C)n1cc(-c2ccc3ccnn3c2)cn1]

6-Bromopyrazolo[1,5-a]pyridine (0.25 g, 1.27 mmol) and 1-(1-ethoxyethyl)-4-(4,4,5,5-tetramethyl-1,3,2-dioxaborolan-2-yl)-1H-pyrazole (0.37 g, 1.40 mmol) in dioxane (5.0 mL) and water (1.0 mL) was sparged with nitrogen for 20 min. Pd(PPh₃) 4 (37 mg, 0.03 mmol) and K₂CO₃ (351 mg, 2.54 mmol) were added and the mixture was sparged with nitrogen for another 5 min. The yellow solution was heated at 100° C. under nitrogen for 2 h. The reaction was allowed to cool to rt then partitioned between water and DCM. The organic layer was washed with brine, dried with Na₂SO₄, and the solvent was removed under reduced pressure. The residue was suspended in DCM and purified by silica gel chromatography eluting with a gradient of acetone/DCM to give 347 mg (100%) of 6-[1-(1-ethoxylethyl)-1H-pyrazol-4-yl]pyrazolo[1,5-a]pyridine yellow solid. ¹H NMR (400 MHZ, CDCl₃) δ (ppm) 8.64 (s, 1H), 7.95 (d, J=2 Hz, 1H), 7.85 (s, 1H), 7.78 (s, 1H), 7.56 (dd, J=9, 1 Hz, 1H), 6.52 (d, J=2 Hz, 1H), 5.57 (q, J=6 Hz, 1H), 3.53 (dq, J=10, 7 Hz, 1H), 3.42 (dq, J=10, 7 Hz, 1H), 1.73 (d, J=6 Hz, 3H), 1.19 (t, J=7 Hz, 3H). MS (ESI) m/z [M+H]⁺ calcd. for C₁₄H₁₇N₄O⁺, 257.1; found, 257.0; HPLC purity 210 nm: 100.0%; 254 nm: 100.0%.

### Step 2. Preparation n of 3-bromo-6-[1-(1-ethoxylethyl)-1H-pyrazol-4-yl]pyrazolo[1,5-a]pyridine

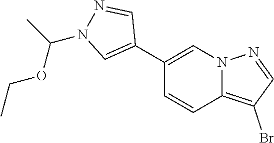 [SMILES: CCOC(C)n1cc(-c2ccc3c(Br)cnn3c2)cn1]

To a solution of 6-[1-(1-ethoxylethyl)-1H-pyrazol-4-yl]pyrazolo[1,5-a]pyridine (0.34 g, 1.32 mmol, as prepared in the previous step) in DMF (3.0 mL) at 0° C. under nitrogen was added NBS (0.28 g, 1.58 mmol) portion-wise at <10° C. After 1 h, a suspension of Na₂S203 (0.52 g, 3.30 mmol) and NaHCO₃ (0.28 g, 3.30 mmol) in water (3 mL) was added and the mixture was stirred at rt for 1 h. The precipitated solid was collected by filtration, washed with water (3 mL×4), and dried under reduced pressure to afford 383 mg (87%) of 3-bromo-6-[1-(1-ethoxylethyl)-1H-pyrazol-4-yl]pyrazolo[1,5-a]pyridine as a solid. ¹H NMR (400 MHZ, CDCl₃) δ (ppm) 8.58 (s, 1H), 7.92 (d, J=2 Hz, 1H), 7.87 (s, 1H), 7.79 (s, 1H), 7.53 (dd, J=9, 1 Hz, 1H), 7.36 (dd, J=9, 1 Hz, 1H), 5.57 (q, J=6 Hz, 1H), 3.53 (dq, J=10, 7 Hz, 1H), 3.43 (dq, J=9, 7 Hz, 1H), 1.73 (d, J=6 Hz, 3H), 1.20 (t, J=7 Hz, 3H). MS (ESI) m/z [M+H]⁺ calcd. for C₁₄H₁₆BrN₄O⁺, 335.1; found, 334.9; HPLC purity: 210 nm: 97.7%; 254 nm: 97.5%.

### Step 3. Preparation of 3-[4-(5-benzylpyrimidin-2-yl)piperazin-1-yl]-6-[1-(1-ethoxyethyl)-1H-pyrazol-4-yl)pyrazolo-[1,5-a]pyridine

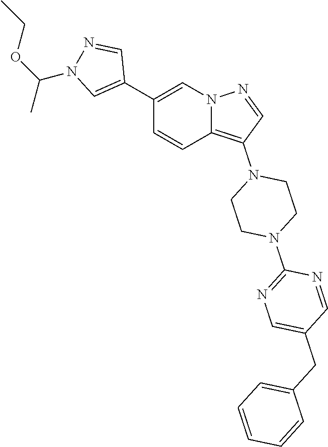 [SMILES: CCOC(C)n1cc(-c2ccc3c(N4CCN(c5ncc(Cc6ccccc6)cn5)CC4)cnn3c2)cn1]

To a mixture of 3-bromo-6-[1-(1-ethoxyethyl)-1H-pyrazol-4-yl]-pyrazolo[1,5-a]pyridine (0.29 g, 0.87 mmol, as prepared in the previous step) and 5-benzyl-2-piperazine-1-ylpyrimidine (Compound S16) (0.26 g, 1.04 mmol) was added tBuOH (3.2 mL) and dioxane (1.6 mL). The solution was sparged with nitrogen for 10 min at 42° C., and then swept with argon atmosphere. To the solution was added NaOtBu (125 mg, 1.30 mmol) and the reaction mixture was swept with argon for 5 min. tBuXPhos Pd G1 (119 mg, 0.17 mmol) was added followed by a 5 min argon sweep. The mixture was heated at 55° C. bath for 3 h, then cooled to rt and filtered through Celite® 545, that was then washed with DCM/MeOH. The filtrate was concentrated under reduced pressure and the residue was purified by silica gel chromatography eluting with EtOAc (0.1% Et₃N)/DCM gradient then further purified by silica gel chromatography eluting with acetone (0.1% Et₃N)/DCM to afford 153 mg (35%) of 3-[4-(5-benzylpyrimidin-2-yl)piperazin-1-yl]-6-[1-(1-ethoxyethyl)-1H-pyrazol-4-yl)pyrazolo-[1,5-a]pyridine solid. ¹H NMR (400 MHZ, CDCl₃) δ (ppm) 8.51 (s, 1H), 8.22 (s, 2H), 7.85 (s, 1H), 7.78 (s, 1H), 7.73 (s, 1H), 7.57 (d, J=9 Hz, 1H), 7.31 (m, 2H), 7.19 (m, 4H), 5.57 (q, J=6 Hz, 1H), 4.01 (m, 4H), 3.82 (s, 2H), 3.53 (dq, J=9, 7 Hz, 1H), 3.42 (dq, J=9, 7 Hz, 1H), 3.13 (m, 4H), 1.73 (d, J=6 Hz, 3H), 1.19 (t, J=7 Hz, 3H). MS (ESI) m/z [M+H]⁺ calcd. for C₂₉H₃₃N₈O⁺, 508.3; found, 509.2; HPLC purity: 210 nm: 99.0%; 254 nm: 99.3%.

### Step 4. Preparation of 3-[4-(5-benzylpyrimidin-2-yl)piperazin-1-yl]-6-(1H-pyrazol-4-yl)pyrazolo[1,5-a]pyridine (Compound 255)

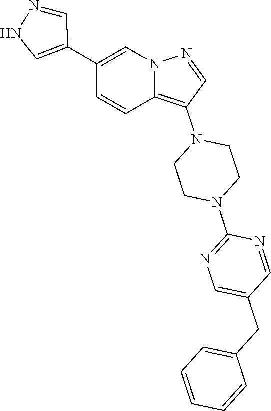 [SMILES: c1ccc(Cc2cnc(N3CCN(c4cnn5cc(-c6cn[nH]c6)ccc45)CC3)nc2)cc1]

To a solution of 3-[4-(5-benzylpyrimidin-2-yl)piperazin-1-yl]-6-[1-(1-ethoxyethyl)-1H-pyrazol-4-yl)pyrazolo-[1,5-a]pyridine (0.15 g, 0.30 mmol, as prepared in the previous step) in THF (1.5 mL) was added 4 M aq. HCl (0.44 mL, 1.76 mmol). The reaction was stirred at rt then basified with 2 M NaOH (0.89 mL, 1.77 mmol). The mixture was partitioned with addition of DCM, H₂O, and sat. aq. NH₄Cl. The organic layer was washed with brine, dried with Na₂SO₄, and concentrated under reduced pressure. The residue was purified by column chromatography eluting with IPA (0.1% Et₃N)/DCM to afford 119 mg (92%) of 3-[4-(5-benzylpyrimidin-2-yl)piperazin-1-yl]-6-(1H-pyrazol-4-yl)pyrazolo[1,5-a]pyridine (Compound 255) as a solid. ¹H NMR (400 MHZ, CDCl₃) δ (ppm) 8.53 (s, 1H) 8.22 (s, 2H), 7.88 (br. s., 2H), 7.74 (s, 1H), 7.59 (d, J=9 Hz, 1H), 7.31 (m, 2H), 7.21 (m, 4H), 4.02 (m, 4H), 3.82 (s, 2H), 3.14 (m, 4H). MS (ESI) m/z [M+H]⁺ calcd. for C₂₅H₂₅N₈⁺, 437.2; found, 437.1; HPLC purity: 210 nm: 98.9%; 254 nm: 99.3%.

### Example 194: Synthesis of Exemplary Compound 256

### 3-(4-(5-(1-(4-fluorophenyl)vinyl)pyrimidin-2-yl)piperazin-1-yl)-6-(1-methyl-1H-pyrazol-4-yl)pyrazolo[1,5-a]pyridine (Compound 256)

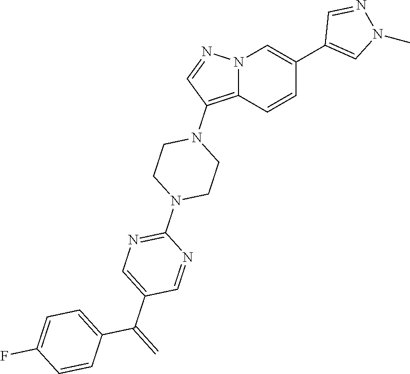 [SMILES: C=C(c1ccc(F)cc1)c1cnc(N2CCN(c3cnn4cc(-c5cnn(C)c5)ccc34)CC2)nc1]

### Step 1. Preparation of 5-(1-(4-fluorophenyl)vinyl)-2-(piperazin-1-yl)pyrimidine

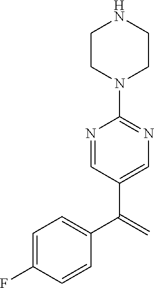 [SMILES: C=C(c1ccc(F)cc1)c1cnc(N2CCNCC2)nc1]

A solution of tert-butyl 4-(5-(1-(4-fluorophenyl)vinyl)pyrimidin-2-yl)piperazine-1-carboxylate (Compound S82) (200 mg, 0.520 mmol) in DCM (6.0 mL) was cooled at 0° C., and treated dropwise with TFA (0.40 mL, 5.2 mmol). The reaction was stirred at 0° C. for 20 min then warmed to rt and stirred overnight. The mixture was concentrated under reduced pressure, the residue was dissolved in DCM (15 mL) and concentrated under reduced pressure. The residue was dissolved in MTBE (20 mL) and the solution was washed with sat. aq. NaHCO₃ (2×15 mL) and 0.1N NaOH solution (20 mL). The organic phase was dried over anhydrous MgSO₄ and filtered, then the filtrate was concentrated under reduced pressure to afford 119 mg (80%) of 5-(1-(4-fluorophenyl)vinyl)-2-(piperazin-1-yl)pyrimidine as a colorless solid. ¹H NMR (400 MHZ, DMSO-d₆) δ (ppm) 8.27 (s, 2H), 7.44-7.33 (m, 2H), 7.27-7.14 (m, 2H), 5.46 (s, 1H), 5.37 (s, 1H), 3.71-3.67 (m, 4H), 2.76-2.72 (m, 4H).

### Step 2. Preparation of 3-(4-(5-(1-(4-fluorophenyl)vinyl)pyrimidin-2-yl)piperazin-1-yl)-6-(1-methyl-1H-pyrazol-4-yl)pyrazolo[1,5-a]pyridine (Compound 256)

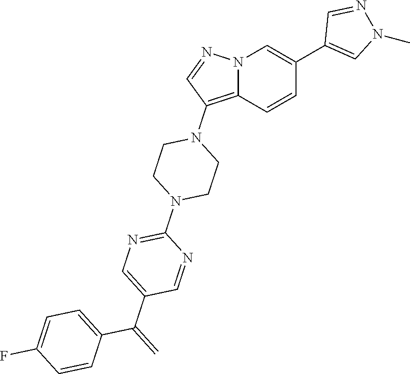 [SMILES: C=C(c1ccc(F)cc1)c1cnc(N2CCN(c3cnn4cc(-c5cnn(C)c5)ccc34)CC2)nc1]

A solution of 3-bromo-6-(1-methyl-1H-pyrazol-4-yl)pyrazolo[1,5-a]pyridine (Compound S9) (149 mg, 0.536 mmol) and 5-[1-(4-fluorophenyl)vinyl]-2-piperazin-1-ylpyrimidine (183 mg, 0.644 mmol, as prepared in the previous step) in tBuOH (1.51 mL) and dioxane (0.76 mL) was sparged with argon for 20 min then tBuXPhos Pd G1 (55.2 mg, 0.0804 mmol) and KOtBu (90.3 mg, 0.804 mmol) were added, and the mixture was sparged again for 10 min with argon. The mixture was stirred at 55° C. under argon for 1.75 h. The reaction was cooled to rt, diluted with EtOAc (40 mL), and washed with water (2×20 mL) and brine (20 mL). The organic phase was dried over anhydrous Na₂SO₄ and filtered, then the filtrate was concentrated under reduced pressure. The residue was purified by flash chromatography eluting with 20-50% acetone/DCM. The crude product was triturated with 25% Et₂O/hexane (5 mL) resulting in a slurry. The solid was isolated by filtration, the solid was washed with 25% Et₂O/hexane, and dried under reduced pressure to afford 151 mg (59%) of 3-(4-(5-(1-(4-fluorophenyl)vinyl)pyrimidin-2-yl)piperazin-1-yl)-6-(1-methyl-1H-pyrazol-4-yl)pyrazolo[1,5-a]pyridine (Compound 256) as a tan solid. ¹H NMR (400 MHZ, CDCl₃) δ (ppm) 8.48 (s, 1H), 8.32 (s, 2H), 7.75 (s, 1H), 7.73 (s, 1H), 7.62 (s, 1H), 7.57 (d, J=8.8 Hz, 1H), 7.36-7.32 (m, 2H), 7.16 (dd, J=9.2, 1.2 Hz, 1H), 7.11-6.99 (m, 2H), 5.38 (s, 1H), 5.34 (s, 1H), 4.14-4.04 (m, 4H), 3.98 (s, 3H), 3.23-3.07 (m, 4H). MS (ESI) m/z [M+H]⁺ calcd. for C₂₇H₂₅FN₈: 481.2, Found: 481.1; HPLC purity: 210 nm: 99.3%; 254 nm: 99.5%.

### Example 195: Synthesis of Exemplary Compound 257

### 2-(4-fluorophenyl)-2-(2-(4-(6-(1-methyl-1H-pyrazol-4-yl)pyrazolo[1,5-a]pyridin-3-yl)piperazin-1-yl)pyrimidin-5-yl)ethan-1-ol (Compound 257)

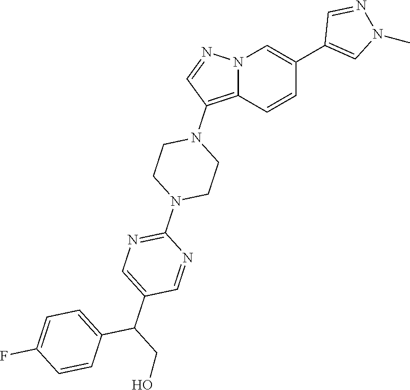 [SMILES: Cn1cc(-c2ccc3c(N4CCN(c5ncc(C(CO)c6ccc(F)cc6)cn5)CC4)cnn3c2)cn1]

### Step 1. Preparation of tert-butyl 4-(5-(1-(4-fluorophenyl)-2-hydroxyethyl)pyrimidin-2-yl)piperazine-1-carboxylate

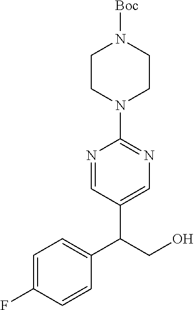 [SMILES: CC(C)(C)OC(=O)N1CCN(c2ncc(C(CO)c3ccc(F)cc3)cn2)CC1]

A solution of tert-butyl 4-{5-[1-(4-fluorophenyl)vinyl]pyrimidin-2-yl}piperazine-1-carboxylate (Compound S82) (700 mg, 1.82 mmol) in THF (8.0 mL) was cooled to 0° C., and treated dropwise with a solution of 0.5 M of 9-BBN in THF (18.2 mL, 9.10 mmol). The mixture was stirred at 0° C. for 2 h, warmed to rt, and stirred for 16.5 h. The reaction was cooled to 0° C. then 3.0 M of NaOH (15 mL, 45 mmol) and 30% H₂O2 (4.129 g, 36.42 mmol) were added. The mixture was stirred at rt for 30 min then H₂O (15 mL) was added, and the mixture was extracted with EtOAc (2×25 mL). The combined organic layers were washed H₂O (20 mL), dried over anhydrous MgSO₄, and filtered. The filtrate was concentrated under reduced pressure then the residue was purified by flash chromatography eluting with 50-60% EtOAc/hexanes to afford 372 mg (51%) of tert-butyl 4-(5-(1-(4-fluorophenyl)-2-hydroxyethyl)pyrimidin-2-yl)piperazine-1-carboxylate as a colorless foam. ¹H NMR (400 MHZ, CDCl₃) δ (ppm) 8.22 (s, 2H), 7.23-7.19 (m, 2H), 7.07-7.00 (m, 2H), 4.16-4.07 (m, 2H), 4.07-4.00 (m, 1H), 3.80-3.76 (m, 4H), 3.51-3.47 (m, 4H), 1.49 (s, 9H). MS (ESI) m/z [M+H]⁺ calcd. for C₂₁H₂₇FN₄O₃: 403.2, Found: 403.3.

### Step 2. Preparation of 5-(1-(4-fluorophenyl)-2-((trimethylsilyl)oxy) ethyl)-2-(piperazin-1-yl)pyrimidine

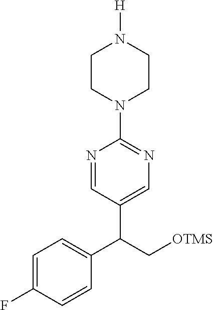

A solution of tert-butyl 4-{5-[1-(4-fluorophenyl)-2-hydroxyethyl]pyrimidin-2-yl}piperazine-1-carboxylate (306 mg, 0.760 mmol, as prepared in the previous step) and 2,6-lutidine (0.370 mL, 3.19 mmol) in DCM (9.0 mL) was cooled to 0° C., and TMSOTf (0.440 mL, 2.43 mmol) was added dropwise. The mixture was stirred at 0° C. for 15 min then warmed to rt and stirred for 2 h. The reaction was cooled to 0° C., sat. aq. NH₄Cl (15 mL) was added and stirred for 5 min. The phases were separated, then the aqueous phase was extracted with MTBE (2×15 mL). The combined organic layers were washed with sat. aq. NaHCO₃ (15 mL) and 0.1N NaOH (2×10 mL), then dried over anhydrous Na₂SO₄ and filtered. The filtrate was concentrated under reduced pressure. The residue was dissolved in MTBE (10 mL) and washed with 0.1N NaOH (2×7 mL). The organic phase was dried over anhydrous Na₂SO₄, and filtered, then the filtrate was concentrated under reduced pressure to afford 250 mg (88%) of 5-(1-(4-fluorophenyl)-2-((trimethylsilyl)oxy) ethyl)-2-(piperazin-1-yl)pyrimidine as a tan viscous oil. ¹H NMR (400 MHZ, CD₃OD) δ (ppm) 8.23 (s, 2H), 7.30-7.25 (m, 2H), 7.07-7.00 (m, 2H), 4.08-3.98 (m, 3H), 3.76-3.72 (m, 4H), 2.87-2.83 (m, 4H), 0.02 (s, 9H).

### Step 3. Preparation of 2-(4-fluorophenyl)-2-(2-(4-(6-(1-methyl-1H-pyrazol-4-yl)pyrazolo[1,5-a]pyridin-3-yl)piperazin-1-yl)pyrimidin-5-yl)ethan-1-ol (Compound 257)

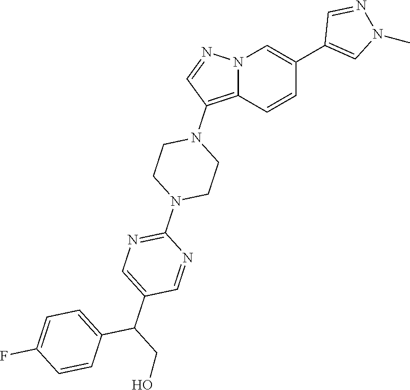 [SMILES: Cn1cc(-c2ccc3c(N4CCN(c5ncc(C(CO)c6ccc(F)cc6)cn5)CC4)cnn3c2)cn1]

A solution of 3-bromo-6-(1-methyl-1H-pyrazol-4-yl)pyrazolo[1,5-a]pyridine (Compound S9) (159 mg, 0.574 mmol) and 5-(1-(4-fluorophenyl)-2-((trimethylsilyl)oxy) ethyl)-2-(piperazin-1-yl)pyrimidine (258 mg, 0.689 mmol, as prepared in the previous step) in tBuOH (1.62 mL) and dioxane (0.81 mL) was sparged with argon for 20 min then tBuXPhos Pd G1 (59.1 mg, 0.0861 mmol) and KOtBu (96.6 mg, 0.861 mmol) were added, and the mixture was sparged for 10 min with argon. The reaction was stirred at 55° C. under argon for 3 h. The reaction was cooled to rt, diluted with EtOAc (30 mL), and washed with water (2×20 mL) and brine (20 mL). The organic phase was dried over anhydrous MgSO₄ and filtered, then the filtrate was concentrated under reduced pressure. The residue was purified by flash chromatography eluting with 30-70% acetone/DCM to afford 55 mg (19%) of 2-(4-fluorophenyl)-2-(2-(4-(6-(1-methyl-1H-pyrazol-4-yl)pyrazolo[1,5-a]pyridin-3-yl)piperazin-1-yl)pyrimidin-5-yl)ethan-1-ol (Compound 257) as a light yellow foam. ¹H NMR (400 MHZ, CDCl₃) δ (ppm) 8.47 (s, 1H), 8.26 (s, 2H), 7.75 (s, 1H), 7.72 (s, 1H), 7.62 (s, 1H), 7.56 (d, J=9.17 Hz, 1H), 7.26-7.19 (m, 2H), 7.15 (dd, J=9.2, 1.3 Hz, 1H), 7.09-6.99 (m, 2H), 4.12 (br s, 2H), 4.07-4.00 (m, 5H), 3.98 (s, 3H), 3.18-3.08 (m, 4H). MS (ESI) m/z [M+H]⁺ calcd. for C₂₇H₂₇FN₈O: 499.2, Found: 499.3; HPLC purity: 210 nm: 98.6%; 254 nm: 98.5%.

### Example 196: Synthesis of Exemplary Compound 258

### 3-(4-(5-(1-(4-fluorophenyl)ethyl)pyrimidin-2-yl)piperazin-1-yl)-6-(1-methyl-1H-pyrazol-4-yl)pyrazolo[1,5-a]pyridine (Compound 258)

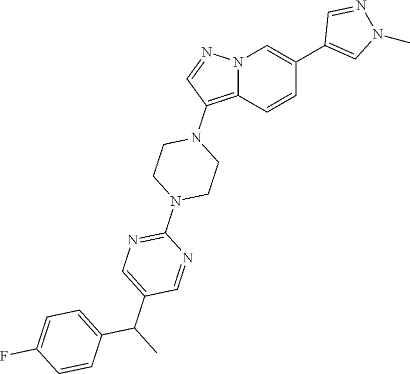 [SMILES: CC(c1ccc(F)cc1)c1cnc(N2CCN(c3cnn4cc(-c5cnn(C)c5)ccc34)CC2)nc1]

### Step 1. Preparation of tert-butyl 4-(5-(1-(4-fluorophenyl)ethyl)pyrimidin-2-yl)piperazine-1-carboxylate

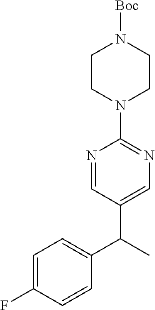 [SMILES: CC(c1ccc(F)cc1)c1cnc(N2CCN(C(=O)OC(C)(C)C)CC2)nc1]

To a solution of tert-butyl 4-{5-[1-(4-fluorophenyl)vinyl]pyrimidin-2-yl}piperazine-1-carboxylate (Compound S82) (275 mg, 0.715 mmol) in EtOH (11 mL) and THF (7.5 mL) was added 10% Pd—C(90 mg, 0.085 mmol). The reaction was stirred under an atmosphere of H₂ at rt for 1.25 h. The reaction was filtered through a pad of celite, the pad was washed with EtOAc (30 mL) and the filtrate was concentrated under reduced pressure. The residue was purified by flash chromatography eluting with 5-25% EtOAc/hexanes to afford 264 mg (96%) of tert-butyl 4-(5-(1-(4-fluorophenyl)ethyl)pyrimidin-2-yl)piperazine-1-carboxylate as a white solid. ¹H NMR (400 MHZ, CDCl₃) δ (ppm) 8.17 (s, 2H), 7.17-7.14 (m, 2H), 7.02-6.96 (m, 2H), 3.99 (q, J=7.2 Hz, 1H), 3.79-3.75 (m, 4H), 3.51-3.47 (m, 4H), 1.60 (d, J=7.2 Hz, 3H), 1.49 (s, 9H).

### Step 3. Preparation of 5-(1-(4-fluorophenyl)ethyl)-2-(piperazin-1-yl)pyrimidine

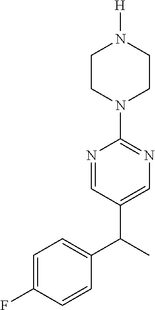 [SMILES: CC(c1ccc(F)cc1)c1cnc(N2CCNCC2)nc1]

To a solution of tert-butyl 4-{5-[1-(4-fluorophenyl)ethyl]pyrimidin-2-yl}piperazine-1-carboxylate (200 mg, 0.518 mmol, as prepared in the previous step) in DCM (5.0 mL) at 0° C. was added TFA (0.400 mL, 5.19 mmol) dropwise. The reaction was stirred at 0° C. for 20 min then warmed to rt and stirred for 21 h. The mixture was concentrated under reduced pressure, then the residue was diluted with DCM (10 mL) and concentrated again. The residue was dissolved in DCM (25 mL), washed with sat. aq. NaHCO₃ (25 mL), 0.1N NaOH solution (20 mL), dried over anhydrous Na₂SO₄, and filtered. The filtrate was concentrated under reduced pressure. The residue was dissolved in MTBE (20 mL), washed with sat. aq. NaHCO₃ (20 mL), 0.1N NaOH solution (10 mL), dried over anhydrous MgSO₄, and filtered. The filtrate was concentrated under reduced pressure affording 123 mg (83%) of 5-(1-(4-fluorophenyl)ethyl)-2-(piperazin-1-yl)pyrimidine as a colorless glass. ¹H NMR (400 MHZ, DMSO-d₆) δ (ppm) 8.25 (s, 2H), 7.33-7.28 (m, 2H), 7.14-7.08 (m, 2H), 4.04 (q, J=7.2 Hz, 1H), 3.61-3.57 (m, 4H), 2.72-2.67 (m, 4H), 1.54 (d, J=7.2 Hz, 3H).

### Step 4. Preparation of 3-(4-(5-(1-(4-fluorophenyl)ethyl)pyrimidin-2-yl)piperazin-1-yl)-6-(1-methyl-1H-pyrazol-4-yl)pyrazolo[1,5-a]pyridine (Compound 258)

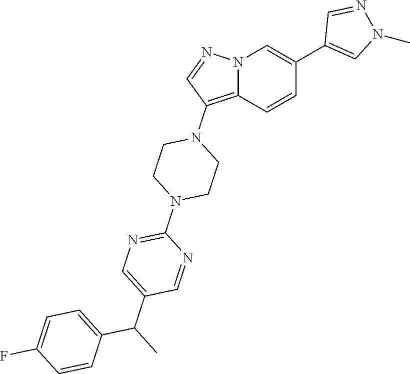 [SMILES: CC(c1ccc(F)cc1)c1cnc(N2CCN(c3cnn4cc(-c5cnn(C)c5)ccc34)CC2)nc1]

A solution of 3-bromo-6-(1-methyl-1H-pyrazol-4-yl)pyrazolo[1,5-a]pyridine (Compound S9) (148 mg, 0.532 mmol) and 5-[1-(4-fluorophenyl)ethyl]-2-piperazin-1-ylpyrimidine (183 mg, 0.639 mmol, as prepared in the previous step) in tBuOH (1.50 mL) and dioxane (0.75 mL) was sparged with argon for 20 min then tBuXPhos Pd G1 (54.8 mg, 0.0799 mmol) and KOtBu (89.6 mg, 0.799 mmol) were added, and the mixture was sparged for 10 min with argon. The reaction was stirred at 55° C. under argon for 2 h. The reaction was cooled to rt, diluted with EtOAc (40 mL), and washed with water (2×20 mL) and brine (20 mL). The organic phase was dried over anhydrous Na₂SO₄ and filtered, then the filtrate was concentrated under reduced pressure. The residue was purified by flash chromatography eluting with 20-50% acetone/DCM. The crude product was triturated with 25% Et₂O/hexane (5 mL) resulting in a slurry. The solid was isolated by filtration, the solid was washed with 25% Et₂O/hexane, and dried under reduced pressure to afford 142 mg (55%) of 3-(4-(5-(1-(4-fluorophenyl)ethyl)pyrimidin-2-yl)piperazin-1-yl)-6-(1-methyl-1H-pyrazol-4-yl)pyrazolo[1,5-a]pyridine (Compound 258) as a tan solid. ¹H NMR (400 MHZ, CDCl₃) δ (ppm) 8.47 (s, 1H), 8.20 (s, 2H), 7.75 (s, 1H), 7.72 (s, 1H), 7.62 (s, 1H), 7.56 (d, J=9.6 Hz, 1H), 7.20-7.13 (m, 3H), 7.00 (t, J=8.6 Hz, 2H), 4.05-3.98 (m, 5H), 3.98 (s, 3H), 3.15-3.10 (m, 4H). MS (ESI) m/z [M+H]⁺ calcd. for C₂₇H₂₇FN₈: 483.2, Found: 483.2; HPLC purity: 210 nm: 99.5%; 254 nm: 100.0%.

### Example 197: Synthesis of Exemplary Compound 259

### 3-[4-(5-benzylpyrimidin-2-yl)piperazin-1-yl]-6-methoxypyrazolo-[1,5-a]pyridine (Compound 259)

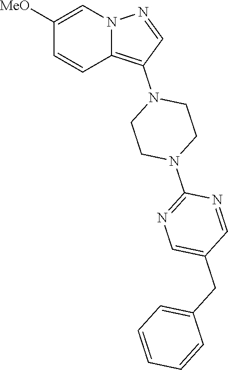 [SMILES: COc1ccc2c(N3CCN(c4ncc(Cc5ccccc5)cn4)CC3)cnn2c1]

A solution of 3-bromo-6-methoxypyrazolo[1,5-a]pyridine (0.20 g, 0.88 mmol) and 5-benzyl-2-piperazine-1-ylpyrimidine (Compound S32) (0.27 g, 1.06 mmol) in tBuOH (3.2 mL) and dioxane (1.6 mL) was sparged with nitrogen for 10 min at 42° C., and then swept with argon atmosphere. To the solution was added NaOtBu (127 mg, 1.32 mmol) and the reaction mixture was swept with argon for 5 min. tBuXPhos Pd G1 (121 mg, 0.18 mmol) was added followed by a 5 min argon sweep. The reaction was heated at 55° C. bath for 2 h and then cooled to rt. The mixture was partitioned between DCM, water, and sat. aq. NH₄Cl. The organic layer was washed with brine, dried with Na₂SO₄, and concentrated under reduced pressure. The residue was purified by) silica gel chromatography cartridge eluting with EtOAc (0.1% Et₃N)/hexane then further purified by silica gel chromatography eluting with acetone (0.1% Et₃N)/hexane to afford 105 mg (30%) of 3-[4-(5-benzylpyrimidin-2-yl)piperazin-1-yl]-6-methoxypyrazolo-[1,5-a]pyridine (Compound 259) as a solid. ¹H NMR (400 MHz, CDCl₃) δ (ppm) 8.21 (s, 2H), 7.95 (d, J=2 Hz, 1H), 7.62 (s, 1H), 7.44 (d, J=10 Hz, 1H), 7.31 (m, 2H), 7.23 (d, J=7 Hz, 1H), 7.19 (m, 2H), 6.85 (dd, J=10, 2, 1H), 3.99 (m, 4H), 3.83 (s, 4H), 3.82 (s, 2H), 3.10 (m, 4H). MS (ESI) m/z [M+H]⁺ calcd. for C₂₃H₂₅N₆O⁺, 401.2; found, 401.1; HPLC purity: 210 nm: 97.4%; 254 nm: 98.3%.

### Example 198: Synthesis of Exemplary Compound 260

### (1R)-1-pyridin-2-ylethyl 4-[6-(1-methyl-1H-pyrazol-4-yl)pyrazolo[1,5-a]pyridin-3-yl]piperazine-1-carboxylate (Compound 260)

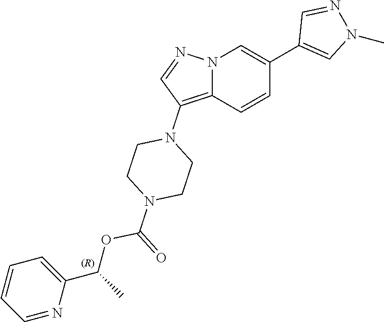

### Step 1. Preparation of 4-nitrophenyl-4-[6-(1-methyl-1H-pyrazol-4-yl)pyrazolo[1,5-a]pyridin-3-yl]piperazine-1-carboxylate

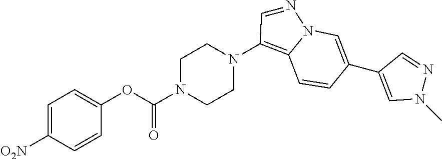 [SMILES: Cn1cc(-c2ccc3c(N4CCN(C(=O)Oc5ccc([N+](=O)[O-])cc5)CC4)cnn3c2)cn1]

A solution of 4-nitrophenyl chloroformate (190 mg, 0.94 mmol), 6-(1-methyl-1H-pyrazol-4-yl)-3-piperazin-1-ylpyrazolo[1,5-a]pyridine hydrochloride (Compound S19) (150 mg, 0.47 mmol), DCM (6.0 mL), and DIPEA (2.0 mL, 11.76 mmol) was stirred at rt for 1 h then the solution was concentrated under reduced pressure. The residue was purified by silica gel chromatography with acetone (0.1% Et₃N)/hexane gradient to yield 51 mg (24%) of 4-nitrophenyl-4-[6-(1-methyl-1H-pyrazol-4-yl)pyrazolo[1,5-a]pyridin-3-yl]piperazine-1-carboxylate as a solid. ¹H NMR (400 MHZ, CDCl₃) δ (ppm) 8.50 (br s, 1H), 8.29 (m, 2H), 7.76 (s, 2H), 7.63 (br s, 1H), 7.59 (br s, 1H), 7.35 (m, 2H), 7.21 (br s, 1H), 3.99 (s, 3H), 3.94 (br s, 2H), 3.84 (br s, 2H), 3.18 (br s, 4H). MS (ESI) m/z [M+H]⁺ calcd. for C₂₂H₂₂N₇O₄: 448.2, Found: 448.0; HPLC purity: 210 nm: 99.1%; 254 nm: 99.0%.

### Step 2. Preparation of (1R)-1-pyridin-2-ylethyl 4-[6-(1-methyl-1H-pyrazol-4-yl)pyrazolo[1,5-a]pyridin-3-yl]piperazine-1-carboxylate (Compound 260)

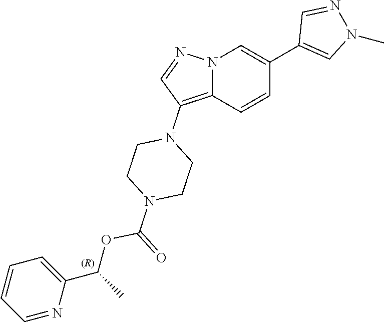

To a mixture of 4-nitrophenyl-4-[6-(1-methyl-1H-pyrazol-4-yl)pyrazolo[1,5-a]pyridin-3-yl]piperazine-1-carboxylate (51 mg, 0.107 mmol, as prepared in the previous step) and (1R)-1-pyridin-2-ylethanol (14 mg, 0.12 mmol) in THF (2.0 mL) was added 60% NaH in mineral oil (30 mg, 0.75 mmol). The reaction was stirred for 4 h then was quenched with sat. aq. NH₄Cl and the mixture was partitioned between water and DCM. The organic layer was washed with brine, dried with Na₂SO₄, and concentrated under reduced pressure. The residue was purified by silica gel chromatography to yield 33 mg (71%) of (1R)-1-pyridin-2-ylethyl 4-[6-(1-methyl-1H-pyrazol-4-yl)pyrazolo[1,5-a]pyridin-3-yl]piperazine-1-carboxylate (Compound 260) as a solid. ¹H NMR (400 MHZ, CDCl₃) δ (ppm) 8.62 (d, J=4 Hz, 1H), 8.47 (s, 1H), 7.75 (s, 1H), 7.72 (br s, 1H), 7.62 (s, 1H), 7.52 (d, J=5 Hz, 1H), 7.38 (d, J=8 Hz, 1H), 7.24 (m, 1H), 7.16 (d J=9 Hz, 1H), 5.91 (q, J=7 Hz, 1H), 3.98 (s, 3H), 3.76 (br s, 4H), 3.07 (br s, 4H), 1.66 (d, J=7 Hz, 4H). MS (ESI) m/z [M+H]⁺ calcd. For C₂₃H₂₆N₇O₂: 432.2, Found: 432.2; HPLC purity: 210 nm: 97.8%; 254 nm: 100.0%.

Using the procedures described in Example 198, Compound 260 and reagents, starting materials, and conditions known to those skilled in the art were used to prepare the compounds shown in Table FF.

TABLE FF

| Compound | Name | Characterization |
|---|---|---|
| Compound | 1-(5-methylpyridin-2-yl)ethyl 4-[6-(1- | ¹H NMR (400 MHz, DMSO- |
| 261 | methyl-1H-pyrazol-4-yl)pyrazolo[1,5- | d₆) δ (ppm) 8.83 (s, 1H), 8.38 |
| | a]pyridin-3-yl]piperazine-1-carboxylate | (d, J = 2.1 Hz, 2H), 8.23 (s, |
| | | 3H), 7.97 (s, 1H), 7.78 (s, 1H), |
| | | 7.64 (m, 2H), 7.32 (m, 2H), |
| | | 5.70 (q, J = 6.6 Hz, 1H), 3.97 |
| | | (s, 3H), 3.61 (br s, 4H), 2.99 |
| | | (br s, 4H), 2.30 (s, 3H), 1.51 |
| | | (d, J = 6.6 Hz, 3H). MS (ESI) |
| | | m/z [M + H]⁺ calcd. for |
| | | C₂₄H₂₈N₇O₂: 446.2, Found: |
| | | 446.3; HPLC purity: 210 nm: |
| | | 100.0%; 254 nm: 100.0%. |

### Example 199: Synthesis of Exemplary Compound 262

### 3-(4-(5-(difluoro (4-fluorophenyl)methyl)pyrimidin-2-yl)piperazin-1-yl)-6-(1-methyl-1H-pyrazol-4-yl)pyrazolo[1,5-a]pyridine (Compound 262)

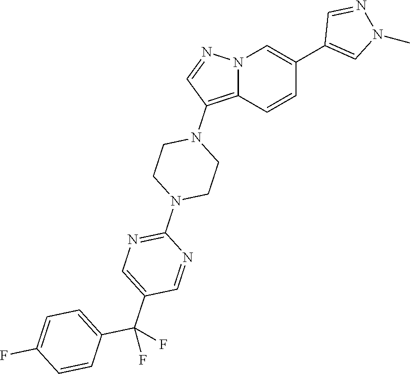 [SMILES: Cn1cc(-c2ccc3c(N4CCN(c5ncc(C(F)(F)c6ccc(F)cc6)cn5)CC4)cnn3c2)cn1]

### Step 1. Preparation of tert-butyl 4-(5-(difluoro (4-fluorophenyl)methyl)pyrimidin-2-yl)piperazine-1-carboxylate

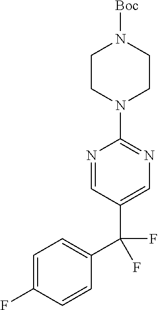 [SMILES: CC(C)(C)OC(=O)N1CCN(c2ncc(C(F)(F)c3ccc(F)cc3)cn2)CC1]

A mixture of tert-butyl 4-[5-(4-fluorobenzoyl)pyrimidin-2-yl]piperazine-1-carboxylate (300 mg, 0.776 mmol, as prepared in Example 28, Step 4) and diethylaminosulfur trifluoride (DAST) (5.5 mL, 42 mmol) was heated in a sealed reaction vial at 90° C. for 17 h behind a safety shield, then the reaction was cooled to rt and diluted with EtOAc (100 mL). The mixture poured into a 500 mL separatory funnel and was treated with sat. aq. NaHCO₃ (7×25 mL), allowing the gas evolution to subside before adding the next portion. Once the off gassing ended, the mixture was carefully shaken, the phases were separated, and the organic phase was washed with sat. aq. NaHCO₃ (50 mL). The organic phase was dried over anhydrous MgSO₄ and filtered, then the filtrate was concentrated under reduced pressure. The residue was purified by flash chromatography eluting with 5-20% EtOAc/hexanes to afford 235 mg (74%) of tert-butyl 4-(5-(difluoro (4-fluorophenyl)methyl)pyrimidin-2-yl)piperazine-1-carboxylate as a light brown solid. ¹H NMR (400 MHZ, CDCl₃) δ (ppm) 8.35 (s, 1H), 7.53-7.48 (m, 2H), 7.20-7.11 (m, 2H), 3.88-3.84 (m, 4H), 3.52-3.48 (m, 4H), 1.50 (s, 9H).

### Step 2. Preparation of 5-(difluoro (4-fluorophenyl)methyl)-2-(piperazin-1-yl)pyrimidine

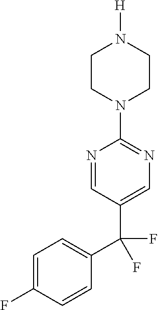 [SMILES: Fc1ccc(C(F)(F)c2cnc(N3CCNCC3)nc2)cc1]

A solution of tert-butyl 4-{5-[difluoro (4-fluorophenyl)methyl]pyrimidin-2-yl}piperazine-1-carboxylate (196 mg, 0.480 mmol, as prepared in the previous step) and 2,6-lutidine (0.178 mL, 1.54 mmol) in DCM (4.9 mL) at 0° C. was treated with TMSOTf (0.191 mL, 1.06 mmol). for the reaction was stirred for 15 min at 0° C., then 1.5 h at rt. The reaction mixture was cooled to 0° C., treated with sat. aq. NH₄Cl (14 mL), and the mixture was extracted with EtOAc (2×20 mL). The combined organic layers were washed with sat. aq. NaHCO₃ (10 mL), dried over anhydrous Na₂SO₄, and filtered. The filtrate was concentrated under reduced pressure. The residue was dissolved in MTBE (15 mL), washed with 0.1N NaOH solution (2×10 mL), dried over anhydrous Na₂SO₄, and filtered. The filtrate was concentrated under reduced pressure to afford 136 mg (92%) of 5-(difluoro (4-fluorophenyl)methyl)-2-(piperazin-1-yl)pyrimidine as a sticky brown solid. ¹H NMR (400 MHZ, CD₃OD) δ (ppm) 8.35 (s, 2H), 7.57 (dd, J=8.8, 5.2 Hz, 2H), 7.23 (t, J=8.8 Hz, 2H), 3.99-3.71 (m, 4H), 2.95-2.77 (m, 4H).

### Step 3. Preparation of 3-(4-(5-(difluoro (4-fluorophenyl)methyl)pyrimidin-2-yl)piperazin-1-yl)-6-(1-methyl-1H-pyrazol-4-yl)pyrazolo[1,5-a]pyridine (Compound 262)

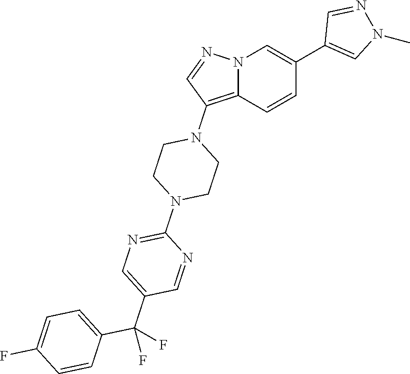 [SMILES: Cn1cc(-c2ccc3c(N4CCN(c5ncc(C(F)(F)c6ccc(F)cc6)cn5)CC4)cnn3c2)cn1]

A solution of 3-bromo-6-(1-methyl-1H-pyrazol-4-yl)pyrazolo[1,5-a]pyridine (Compound S9) (120 mg, 0.435 mmol) and 5-[difluoro (4-fluorophenyl)methyl]-2-piperazin-1-ylpyrimidine (161 mg, 0.522 mmol, as prepared in the previous step) in tBuOH (1.22 mL) and dioxane (0.61 mL) was sparged with argon for 20 min then tBuXPhos Pd G1 (44.8 mg, 0.0653 mmol) and KOtBu (73.2 mg, 0.653 mmol) were added, and the mixture was sparged for 10 min with argon. The reaction was stirred at 55° C. under argon for 3.3 h then additional dioxane (0.8 mL), tBuXPhos Pd G1 (44.8 mg, 0.0653 mmol) and KOtBu (73.2 mg, 0.653 mmol) were added, and the mixture was sparged with argon for 15 min. The reaction was stirred at 55° C. for 17.5 h, cooled to rt, and diluted with EtOAc (30 mL) and H₂O (20 mL). The mixture was filtered through a pad of magnasol and the pad was washed with additional EtOAc and H₂O. The phases were separated, and the organic phase was washed with H₂O (20 mL), dried over anhydrous MgSO₄, and filtered. The filtrate was concentrated under reduced pressure then the residue was purified by flash chromatography eluting with 10-40% acetone/DCM. The resulting brown solid was triturated with 10% Et₂O/hexane (2×), then the solid was dissolved in DCM, and the solution was concentrated to afford 67 mg (30%) of 3-(4-(5-(difluoro (4-fluorophenyl)methyl)pyrimidin-2-yl)piperazin-1-yl)-6-(1-methyl-1H-pyrazol-4-yl)pyrazolo[1,5-a]pyridine (Compound 262) as a brown solid. ¹H NMR (400 MHZ, CDCl₃) δ (ppm) 8.48 (s, 1H), 8.38 (s, 2H), 7.75 (s, 1H), 7.73 (s, 1H), 7.62 (s, 1H), 7.61-7.54 (m, 1H), 7.55-7.50 (m, 2H), 7.22-7.09 (m, 3H), 4.11 (br s, 3H), 3.98 (s, 4H), 3.15 (br s, 4H). MS (ESI) m/z [M+H]⁺ calcd. for C₂₆H₂₃F₃N₈: 505.2, Found: 505.3; HPLC purity: 210 nm: 97.7%; 254 nm: 98.4%.

### Example 200: Synthesis of Exemplary Compound 263

### 3-(4-(5-((4-fluorophenyl)(methoxy)methyl)pyrimidin-2-yl)piperazin-1-yl)-6-(1-methyl-1H-pyrazol-4-yl)pyrazolo[1,5-a]pyridine (Compound 263)

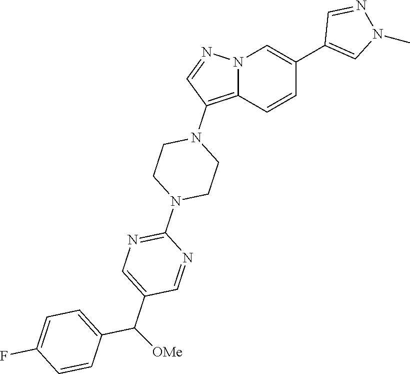 [SMILES: COC(c1ccc(F)cc1)c1cnc(N2CCN(c3cnn4cc(-c5cnn(C)c5)ccc34)CC2)nc1]

### Step 1. Preparation of tert-butyl 4-(5-((4-fluorophenyl)(hydroxy)methyl)pyrimidin-2-yl)piperazine-1-carboxylate

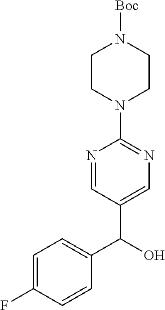 [SMILES: CC(C)(C)OC(=O)N1CCN(c2ncc(C(O)c3ccc(F)cc3)cn2)CC1]

A mixture of tert-butyl 4-[5-(4-fluorobenzoyl)pyrimidin-2-yl]piperazine-1-carboxylate (500 mg, 1.29 mmol, as prepared in Example 28, Step 4) in MeOH (18 mL) and THF (3 mL) was treated with NaBH₄ (171 mg, 4.53 mmol) in one portion and the reaction was stirred at rt for 4 h. The mixture was concentrated to ½ the original volume and sat. aq. NaHCO₃ (15 mL) and H₂O (10 mL) were added. The mixture was extracted with EtOAc (3×15 mL) then the combined organic layers were washed with H₂O (20 mL), dried over anhydrous MgSO₄, filtered, and the filtrate was concentrated under reduced pressure to afford 501 mg (100%) of tert-butyl 4-(5-((4-fluorophenyl)(hydroxy)methyl)pyrimidin-2-yl)piperazine-1-carboxylate as a colorless foam. ¹H NMR (400 MHz, CDCl₃) δ (ppm) 8.26 (s, 2H), 7.37-7.34 (m, 2H), 7.05 (t, J=8.6 Hz, 2H), 5.73 (s, 1H), 3.81-3.77 (m, 4H), 3.49-3.46 (m, 4H), 1.49 (s, 9H).

### Step 2. Preparation of 5-((4-fluorophenyl)(methoxy)methyl)-2-(piperazin-1-yl)pyrimidine

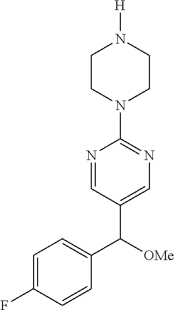 [SMILES: COC(c1ccc(F)cc1)c1cnc(N2CCNCC2)nc1]

A solution tert-butyl 4-(5-((4-fluorophenyl)(hydroxy)methyl)pyrimidin-2-yl)piperazine-1-carboxylate (256 mg, 0.659 mmol, as prepared in the previous step) in MeOH (13 mL) was treated dropwise with a solution of H₂SO₄ (162 mg, 1.65 mmol) in MeOH (3 mL) and the solution was stirred at rt for 66 h. Additional H2SO₄ (100 mg, 1.02 mmol) in MeOH (1 mL) was added and the reaction was stirred at rt for 11 days. The reaction mixture was concentrated to ˜3/4 of the original volume and diluted with H₂O (10 mL) and sat. aq. NaHCO₃ (10 mL). IN NaOH solution was added dropwise to obtain a pH of 10-11. The aqueous phase was extracted with DCM (3×15 mL) then the combined organic layers were dried over anhydrous Na₂SO₄, filtered, and the filtrate was concentrated under reduced pressure to afford 177 mg (89%) of 5-((4-fluorophenyl)(methoxy)methyl)-2-(piperazin-1-yl)pyrimidine as a light tan viscous liquid. ¹H NMR (400 MHZ, CD₃OD) δ (ppm) 8.21 (s, 1H), 7.37 (t, J=8.0 Hz, 1H), 7.09 (t, J=8.4 Hz, 1H), 5.22 (s, 1H), 3.67-3.84 (m, 4H), 3.33 (s, 3H), 2.76-2.93 (m, 4H).

### Step 3. Preparation of 3-(4-(5-((4-fluorophenyl)(methoxy)methyl)pyrimidin-2-yl)piperazin-1-yl)-6-(1-methyl-1H-pyrazol-4-yl)pyrazolo[1,5-a]pyridine (Compound 263)

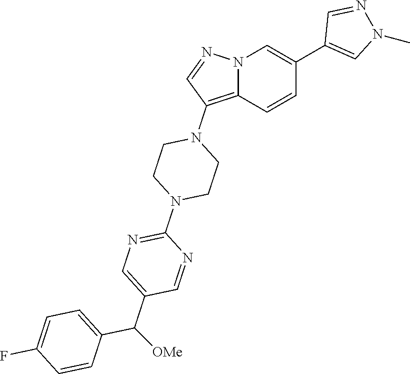 [SMILES: COC(c1ccc(F)cc1)c1cnc(N2CCN(c3cnn4cc(-c5cnn(C)c5)ccc34)CC2)nc1]

A solution of 3-bromo-6-(1-methyl-1H-pyrazol-4-yl)pyrazolo[1,5-a]pyridine (Compound S9) (130 mg, 0.468 mmol) and 5-[(4-fluorophenyl)(methoxy)methyl]-2-piperazin-1-ylpyrimidine (170.0 mg, 0.5623 mmol, as prepared in the previous step) in tBuOH (1.32 mL) and dioxane (0.66 mL) was sparged with argon for 20 min. tBuXPhos Pd G1 (48.3 mg, 0.0703 mmol) and KOtBu (78.9 mg, 0.703 mmol) were added, and the mixture was sparged with argon for 10 min. The reaction was stirred at 55° C. under argon for 1.75 h then cooled to rt and diluted with EtOAc (30 mL). The mixture was washed with H₂O (2×20 mL), the organic phase was dried over anhydrous Na₂SO₄ and filtered. The filtrate was concentrated under reduced pressure. The residue was purified by flash chromatography eluting with 20-50% acetone/DCM to afford 98 mg (42%) of 3-(4-(5-((4-fluorophenyl)(methoxy)methyl)pyrimidin-2-yl)piperazin-1-yl)-6-(1-methyl-1H-pyrazol-4-yl)pyrazolo[1,5-a]pyridine (Compound 263) as a light yellow foam. ¹H NMR (400 MHZ, CDCl₃) δ (ppm) 8.47 (s, 1H), 8.25 (s, 2H), 7.75 (s, 1H), 7.72 (s, 1H), 7.62 (s, 1H), 7.56 (br d, J=9.5 Hz, 1H), 7.32 (dd, J=8.6, 5.4 Hz, 2H), 7.16 (d, J=9.5 Hz, 1H), 7.06 (t, J=8.6 Hz, 2H), 5.13 (s, 1H), 4.04 (br s, 4H), 3.98 (s, 3H), 3.37 (s, 3H), 3.13 (br s, 4H). MS (ESI) m/z [M+H]⁺ calcd. for C₂₇H₂₇FN₈O: 499.2, Found: 499.2; HPLC purity: 210 nm: 99.3%; 254 nm: 99.6%.

### Example 201: Synthesis of Exemplary Compound 264

### (4-fluorophenyl)(2-(4-(6-(1-methyl-1H-pyrazol-4-yl)pyrazolo[1,5-a]pyridin-3-yl)piperazin-1-yl)pyrimidin-5-yl)methyl acetate (Compound 264)

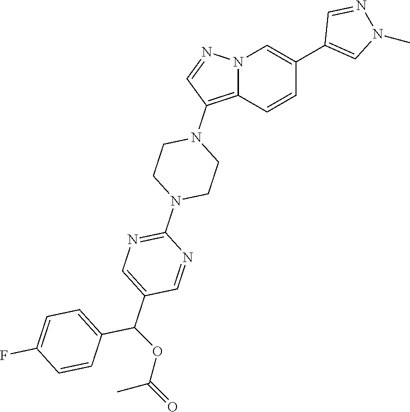 [SMILES: CC(=O)OC(c1ccc(F)cc1)c1cnc(N2CCN(c3cnn4cc(-c5cnn(C)c5)ccc34)CC2)nc1]

A solution of (4-fluorophenyl)(2-{4-[6-(1-methyl-1H-pyrazol-4-yl)pyrazolo[1,5-a]pyridin-3-yl]piperazin-1-yl}pyrimidin-5-yl)methanol (Compound 26) (108 mg, 0.223 mmol) in DCM (4.0 mL) was treated with pyridine (25.2 μL, 0.312 mmol) then Ac₂O (25.2 L, 0.267 mmol) was added dropwise. The reaction mixture was stirred at rt for 1 h. DMAP (10.0 mg, 0.0818 mmol) was added in one portion and stirred at rt for 2 h. The reaction mixture was diluted with DCM (20 mL) and washed with 10 mL of each 0.5 N HCl, sat NaHCO₃, and H₂O and then dried over anhydrous Na₂SO₄. The solution was filtered and concentrated under reduced pressure to yield a yellow viscous oil that was placed under high vacuum. The crude material was purified by chromatography eluting with 20-50% acetone/DCM to afford 97 mg (83%) of (4-fluorophenyl)(2-(4-(6-(1-methyl-1H-pyrazol-4-yl)pyrazolo[1,5-a]pyridin-3-yl)piperazin-1-yl)pyrimidin-5-yl)methyl acetate (Compound 264) as a stiff foam. ¹H NMR (400 MHZ, CDCl₃) δ (ppm) 8.47 (s, 1H), 8.28 (s, 2H), 7.75 (s, 1H), 7.72 (s, 1H), 7.62 (s, 1H), 7.49-7.60 (m, 1H), 7.30-7.39 (m, 2H), 7.16 (d, J=8.68 Hz, 1H), 7.03-7.13 (m, 2H), 6.76 (s, 1H), 4.05 (br. s., 4H), 3.98 (s, 3H), 3.13 (br. s., 4H), 2.15 (s, 3H). MS (ESI) m/z [M+H]⁺ calcd. for C₂₈H₂₇FN₈O₂: 527.2, Found: 527.3; HPLC purity: 210 nm: 99.3%; 254 nm: 99.1%.

### Example 202: Synthesis of Exemplary Compound 265

### 1-(4-chlorophenyl)ethyl 4-(6-(1-methyl-1H-pyrazol-4-yl)pyrazolo[1,5-a]pyridin-3-yl)piperazine-1-carboxylate (Compound 265)

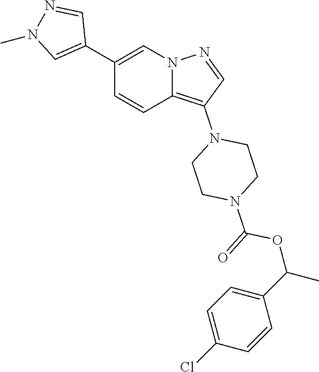 [SMILES: CC(OC(=O)N1CCN(c2cnn3cc(-c4cnn(C)c4)ccc23)CC1)c1ccc(Cl)cc1]

### Step 1. Preparation of 1-(4-chlorophenyl)ethyl (4-nitrophenyl) carbonate

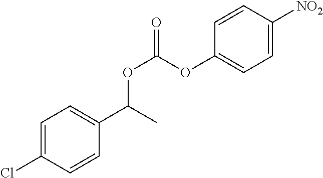 [SMILES: CC(OC(=O)Oc1ccc([N+](=O)[O-])cc1)c1ccc(Cl)cc1]

To a solution of 1-(4-chlorophenyl)ethan-1-ol (250 mg, 1.6 mmol) in DCM (16 mL) was added pyridine (0.542 mL, 6.70 mmol) at 0° C. under nitrogen. The mixture was stirred for 10 min and 4-nitrophenyl chloroformate (0.644 g, 3.19 mmol) was added in one portion at 0° C. and then the reaction was allowed to warm to rt. The suspension was stirred at rt under nitrogen for 28 h, then the mixture was concentrated under reduced pressure. The residue was triturated with 50% EtOAc/hexane (16 mL) for 10 min then the slurry was filtered. The solids were triturated twice more, and the filtrates were combined and then concentrated under reduced pressure. The residue was purified by column chromatography eluting with 10-40% DCM/hexane to afford 517 mg (100%) of 1-(4-chlorophenyl)ethyl (4-nitrophenyl)carbonate as a clear oil. ¹H NMR (400 MHZ, CDCl₃) δ (ppm) 8.27 (d, J=9.6 Hz, 2H), 7.38 (s, 4H), 7.36 (d, J=9.2 Hz, 2H), 5.82 (q, J=6.4 Hz, 1H), 1.69 (d, J=6.4 Hz, 3H).

### Step 2. Preparation of 1-(4-chlorophenyl)ethyl 4-(6-(1-methyl-1H-pyrazol-4-yl)pyrazolo[1,5-a]pyridin-3-yl)piperazine-1-carboxylate (Compound 265)

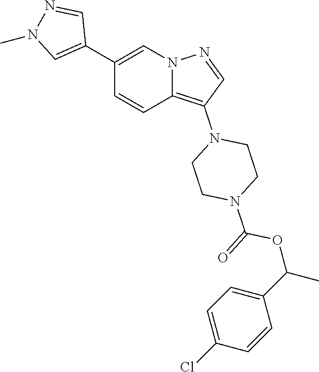 [SMILES: CC(OC(=O)N1CCN(c2cnn3cc(-c4cnn(C)c4)ccc23)CC1)c1ccc(Cl)cc1]

To a mixture of 1-(4-chlorophenyl)ethyl (4-nitrophenyl)carbonate (0.517 g, 1.61 mmol, as prepared in the previous step) and 6-(1-methyl-1H-pyrazol-4-yl)-3-piperazin-1-ylpyrazolo[1,5-a]pyridine hydrochloride salt (Compound S19) (500.0 mg, 1.568 mmol) in ACN (15.0 mL) was added DIPEA (5.464 mL, 31.37 mmol) and the reaction was stirred under nitrogen at rt for 75 min. The mixture was concentrated under reduced pressure then the residue was purified by column chromatography eluting with 20-70% acetone/hexane and 0.1% Et₃N to afford 514 mg (71%) of 1-(4-chlorophenyl)ethyl 4-(6-(1-methyl-1H-pyrazol-4-yl)pyrazolo[1,5-a]pyridin-3-yl)piperazine-1-carboxylate (Compound 265) as a yellow solid. ¹H NMR (400 MHZ, CDCl₃) δ (ppm) 8.47 (s, 1H), 7.75 (s, 1H), 7.72 (s, 1H), 7.62 (s, 1H), 7.54 (br s, 1H), 7.36-7.29 (m, 4H), 7.17 (br d, J=8.8 Hz, 1H), 5.83 (q, J=6.8 Hz, 1H), 3.73 (br s, 4H), 3.06 (br s, 4H), 1.56 (d, J=6.8 Hz, 3H). MS (ESI) m/z [M+H]⁺ calcd. for C₂₄H₂₅ClN₆O₂: 465.2, Found: 465.1; HPLC purity: 210 nm: 98.6%; 254 nm: 99.1%.

### Example 203: Synthesis of Exemplary Compound 266

### (1R)-1-(3,4-difluorophenyl]ethyl 4-[6-(1-methyl-1H-pyrazol-4-yl)pyrazolo[1,5-a]pyridin-3-yl]piperazine-1-carboxylate (Compound 266)

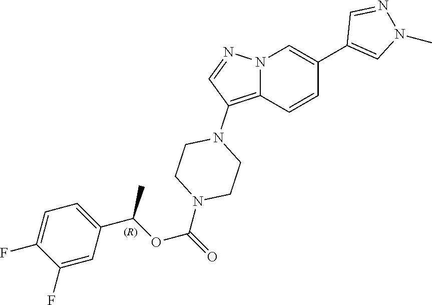

### Step 1. Preparation of (1R)-1-(3,4-difluorophenyl)ethyl 4-nitrophenyl carbonate

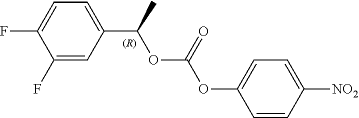

To a solution of (1R)-1-(3,4-difluorophenyl)ethanol (302 mg, 1.91 mmol) in DCM (20 mL) at 3° C. was added pyridine (0.65 mL, 8.0 mmol). The mixture was stirred for 10 min and 4-nitrophenyl chloroformate (0.770 g, 3.82 mmol) was added. The reaction was warmed to rt and stirred overnight. The reaction was concentrated under vacuum. The residue was triturated with 50% EtOAc in hexane (3×15 mL). The filtrate was concentrated under reduced pressure and the residue was purified by ISCO chromatography eluting with a gradient of 0-60% DCM in hexane to afford 0.59 g (96%) of (1R)-1-(3,4-difluorophenyl)ethyl 4-nitrophenyl carbonate as a colorless oil: ¹H NMR (400 MHZ, CDCl₃) δ (ppm) 8.24-8.32 (m, 2H), 7.34-7.42 (m, 2H), 7.24-7.31 (m, 1H), 7.13-7.22 (m, 2H), 5.79 (q, J=6.60 Hz, 1H), 1.69 (d, J=6.60 Hz, 3H).

### Step 2. Preparation of (1R)-1-(3,4-difluorophenyl]ethyl 4-[6-(1-methyl-1H-pyrazol-4-yl)pyrazolo[1,5-a]pyridin-3-yl]piperazine-1-carboxylate (Compound 266)

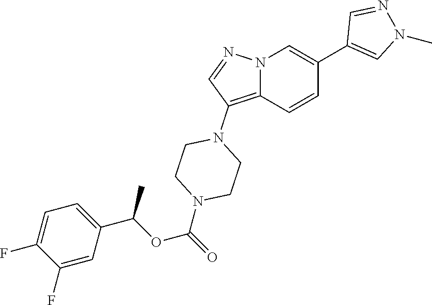 [SMILES: C[C@@H](OC(=O)N1CCN(c2cnn3cc(-c4cnn(C)c4)ccc23)CC1)c1ccc(F)c(F)c1]

To a solution of (1R)-1-(3,4-difluorophenyl)ethyl 4-nitrophenyl carbonate (0.23 g, 0.71 mmol, as prepared in the previous step) and ACN (8.8 mL) was added 6-(1-methyl-1H-pyrazol-4-yl)-3-piperazin-1-ylpyrazolo[1,5-a]pyridine hydrochloride (Compound S19) (0.13 g, 0.41 mmol) and DIPEA (3.2 mL, 18 mmol). The reaction was stirred overnight at rt, then the reaction was concentrated under reduced pressure. The residue was purified by chromatography eluting with a gradient from 20%-70% acetone (containing 0.1% Et₃N) in hexanes to afford 124 mg (65%) of (1R)-1-(3,4-difluorophenyl]ethyl 4-[6-(1-methyl-1H-pyrazol-4-yl)pyrazolo[1,5-a]pyridin-3-yl]piperazine-1-carboxylate (Compound 266) as a pale yellow solid. ¹H NMR (400 MHZ, CDCl₃) δ (ppm) 8.47 (s, 1H), 7.75 (s, 1H), 7.71 (s, 1H), 7.61 (s, 1H), 7.50 (d, J=9.05 Hz, 1H), 7.04-7.23 (m, 4H), 5.80 (q, J=6.60 Hz, 1H), 3.98 (s, 3H), 3.60-3.82 (m, 4H), 2.97-3.12 (m, 4H), 1.56 (d, J=6.60 Hz, 3H). MS (ESI) m/z [M+H]⁺ calcd. for C₂₄H₂₅F₂N₆O₂: 467.2, Found: 467.1; HPLC purity: 210 nm: 100.0%; 254 nm: 100.0%.

Using the procedures described in Example 203, Compound 266 and reagents, starting materials, and conditions known to those skilled in the art were used to prepare the compounds shown in Table GG.

TABLE GG

| Compound | Name | Characterization |
|---|---|---|
| Compound | (1R)-1-(4-methylphenyl)ethyl 4-[6-(1- | ¹H NMR (400 MHz, CDCl₃) δ |
| 267 | methyl-1H-pyrazol-4-yl)pyrazolo[1,5- | (ppm) 8.46 (s, 1H), 7.74 (s, |
| | a]pyridin-3-yl]piperazine-1-carboxylate | 1H), 7.70 (s, 1H), 7.61 (s, 1H), |
| | | 7.50 (d, J = 9.17 Hz, 1H), 7.24- |
| | | 7.34 (m, 2H), 7.11-7.22 (m, |
| | | 3H), 5.83 (q, J = 6.56 Hz, 1H), |
| | | 3.98 (s, 3H), 3.58-3.79 (m, |
| | | 4H), 2.93-3.11 (m, 4H), 2.36 |
| | | (s, 3H), 1.57 (d, J = 6.60 Hz, |
| | | 3H). MS (ESI) m/z [M + H]⁺ |
| | | calcd. for C₂₅H₂₉N₆O₂: 445.2, |
| | | Found: 445.2; HPLC purity: |
| | | 210 nm: 100.0%; 254 nm: |
| | | 100.0%. |

### Example 204: Synthesis of Exemplary Compound 268

### (1R)-1-[4-(trifluoromethoxy)phenyl]ethyl 4-[6-(1-methyl-1H-pyrazol-4-yl)pyrazolo[1,5-a]pyridin-3-yl]piperazine-1-carboxylate (Compound 268)

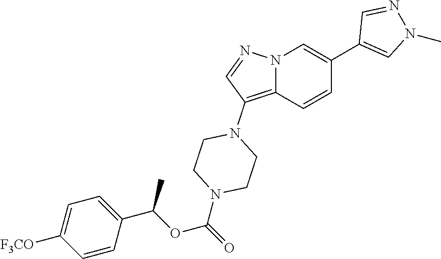 [SMILES: C[C@@H](OC(=O)N1CCN(c2cnn3cc(-c4cnn(C)c4)ccc23)CC1)c1ccc(OC(F)(F)F)cc1]

### Step 1. Preparation of 4-nitrophenyl (1R)-1-[4-(trifluoromethoxy)phenyl]ethyl carbonate

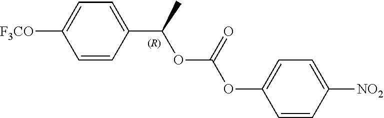

To a solution of (1R)-1-[4-(trifluoromethoxy)phenyl]ethanol (216 mg, 1.05 mmol) in DCM (17 mL) at 3° C. was added pyridine (0.56 mL, 7.0 mmol). The mixture was stirred for 10 min then 4-nitrophenyl chloroformate (0.422 g, 2.10 mmol) was added. The reaction stirred at rt overnight. The reaction was concentrated under vacuum. The residue was purified by ISCO chromatography eluting with a gradient of 0-100% DCM in hexane to afford 0.36 g (93%) of 4-nitrophenyl (1R)-1-[4-(trifluoromethoxy)phenyl]ethyl carbonate as a colorless oil. ¹H NMR (400 MHZ, CDCl₃) δ (ppm) 8.13-8.40 (m, 2H), 7.44-7.51 (m, 2H), 7.34-7.41 (m, 2H), 7.21-7.31 (m, 2H), 5.86 (q, J=6.60 Hz, 1H), 1.71 (d, J=6.60 Hz, 3H).

### Step 2. Preparation of (1R)-1-[4-(trifluoromethoxy)phenyl]ethyl 4-[6-(1-methyl-1H-pyrazol-4-yl)pyrazolo[1,5-a]pyridin-3-yl]piperazine-1-carboxylate (Compound 268)

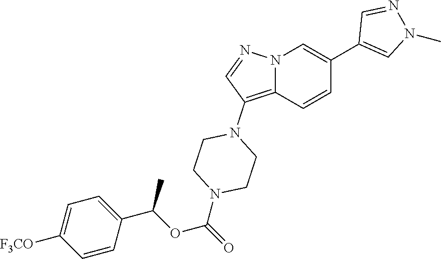 [SMILES: C[C@@H](OC(=O)N1CCN(c2cnn3cc(-c4cnn(C)c4)ccc23)CC1)c1ccc(OC(F)(F)F)cc1]

To a solution of (1R)-1-[4-(trifluoromethoxy)phenyl]ethyl carbonate (0.32 g, 0.87 mmol, as prepared in the previous step) and ACN (7.0 mL) was added 6-(1-methyl-1H-pyrazol-4-yl)-3-piperazin-1-ylpyrazolo[1,5-a]pyridine hydrochloride (Compound S19) (0.16 g, 0.50 mmol) and DIPEA (2.5 mL, 14 mmol). The reaction was stirred overnight at rt, then concentrated in vacuo. The residue was purified by ISCO chromatography eluting with a gradient from 20%-60% acetone (containing 0.1% Et₃N) in hexanes to afford an oil. The residue was stirred with 25% Et₂O in hexanes and concentrated under reduced pressure. This was repeated several times until a solid formed. The solid was dried at 60° C. under reduced pressure to afford 175 mg (68%) of (1R)-1-[4-(trifluoromethoxy)phenyl]ethyl 4-[6-(1-methyl-1H-pyrazol-4-yl)pyrazolo[1,5-a]pyridin-3-yl]piperazine-1-carboxylate (Compound 268) as a white solid. ¹H NMR (400 MHZ, DMSO-d₆) δ (ppm) 8.82 (s, 1H), 8.22 (s, 1H), 7.96 (d, J=0.61 Hz, 1H), 7.77 (s, 1H), 7.65 (dd, J=9.29, 0.61 Hz, 1H), 7.48-7.55 (m, 2H), 7.29-7.41 (m, 3H), 5.78 (q, J=6.52 Hz, 1H), 3.87 (s, 3H), 3.45-3.74 (m, 4H), 2.90-3.06 (m, 4H), 1.49 (d, J=6.60 Hz, 3H). MS (ESI) m/z [M+H]⁺ calcd. for C₂₅H₂₆F₃N₆O₃: 515.2, Found: 515.4; HPLC purity: 210 nm: 100.0%; 254 nm: 100.0%.

Using the procedures described in Example 205, Compound 268 and reagents, starting materials, and conditions known to those skilled in the art were used to prepare the compounds shown in Table HH.

TABLE HH

| Compound | Name | Characterization |
|---|---|---|
| Compound | (1R)-1-(1-naphthyl)ethyl 4-[6-(1-methyl- | ¹H NMR (400 MHz, CDCl₃) δ |
| 269 | 1H-pyrazol-4-yl)pyrazolo[1,5-a]pyridin-3- | (ppm) 8.46 (s, 1H), 8.16 (d, |
| | yl]piperazine-1-carboxylate | J = 8.44 Hz, 1H), 7.89 (d, |
| | | J = 7.70 Hz, 1H), 7.82 (d, |
| | | J = 8.19 Hz, 1H), 7.74 (s, 1H), |
| | | 7.70 (s, 1H), 7.44-7.64 (m, |
| | | 6H), 7.15 (dd, J = 9.17, 1.22 |
| | | Hz, 1H), 6.63 (q, J = 6.60 Hz, |
| | | 1H), 3.98 (s, 3H), 3.65-3.84 |
| | | (m, 4H), 2.92-3.13 (m, 4H), |
| | | 1.76 (d, J = 6.60 Hz, 3H). MS |
| | | (ESI) m/z [M + H]⁺ calcd. for |
| | | C₂₈H₂₉N₆O₂: 481.2, Found: |
| | | 481.2; HPLC purity: 210 nm: |
| | | 100.0%; 254 nm: 100.0%. |
| Compound | (1R)-1-(2,4-dichlorophenyl)ethyl 4-[6-(1- | ¹H NMR (400 MHz, DMSO- |
| 270 | methyl-1H-pyrazol-4-yl)pyrazolo[1,5- | d₆) δ (ppm) 8.82 (s, 1H), 8.22 |
| | a]pyridin-3-yl]piperazine-1-carboxylate | (s, 1H), 7.97 (s, 1H), 7.77 (s, |
| | | 1H), 7.61-7.69 (m, 2H), 7.46- |
| | | 7.56 (m, 2H), 7.33 (dd, |
| | | J = 9.17, 1.47 Hz, 1H), 5.96 (q, |
| | | J = 6.48 Hz, 1H), 3.87 (s, 3H), |
| | | 3.43-3.80 (m, 4H), 2.81- |
| | | 3.11 (m, 4H), 1.48 (d, J = 6.48 |
| | | Hz, 3H). MS (ESI) m/z |
| | | [M + H]⁺ calcd. for |
| | | C₂₄H₂₅Cl₂N₆O₂: 499.1, |
| | | Found: 499.1; HPLC purity: |
| | | 210 nm: 100.0%; 254 nm: |
| | | 100.0%. |
| Compound | (1S)-1-(4-chlorophenyl)ethyl 4-[6-(1- | ¹H NMR (400 MHz, DMSO- |
| 271 | methyl-1H-pyrazol-4-yl)pyrazolo[1,5- | d₆) δ (ppm) 8.83 (s, 1H), 8.23 |
| | a]pyridin-3-yl]piperazine-1-carboxylate | (s, 1H), 7.97 (s, 1H), 7.77 (s, |
| | | 1H), 7.65 (d, J = 9.17 Hz, 1H), |
| | | 7.38-7.49 (m, 4H), 7.33 (dd, |
| | | J = 9.17, 1.34 Hz, 1H), 5.74 (q, |
| | | J = 6.60 Hz, 1H), 3.87 (s, 3H), |
| | | 3.45-3.75 (m, 4H), 2.88- |
| | | 3.06 (m, 4H), 1.48 (d, J = 6.60 |
| | | Hz, 3H). MS (ESI) m/z |
| | | [M + H]⁺ calcd. for |
| | | C₂₄H₂₆ClN₆O₂: 465.2, Found: |
| | | 465.1; HPLC purity: 210 nm: |
| | | 100.0%; 254 nm: 100.0%. |
| Compound | 3-cyanobenzyl 4-[6-(1-methyl-1H-pyrazol- | ¹H NMR (400 MHz, CDCl₃) δ |
| 272 | 4-yl)pyrazolo[1,5-a]pyridin-3- | (ppm) 8.47 (s, 1H), 7.75 (s, |
| | yl]piperazine-1-carboxylate | 1H), 7.67-7.73 (m, 2H), 7.59- |
| | | 7.66 (m, 3H), 7.46-7.55 (m, |
| | | 2H), 7.16 (dd, J = 9.17, 1.47 |
| | | Hz, 1H), 5.20 (s, 2H), 3.98 (s, |
| | | 3H), 3.68-3.79 (m, 4H), 2.99- |
| | | 3.13 (m, 4H). MS (ESI) m/z |
| | | [M + H]⁺ calcd. for |
| | | C₂₄H₂₄N₇O₂: 442.2, Found: |
| | | 442.1; HPLC purity: 210 nm: |
| | | 100.0%; 254 nm: 100.0%. |
| Compound | 1-(3-fluoro-4- | ¹H NMR (400 MHz, DMSO- |
| 273 | (trifluoromethyl)phenyl)ethyl 4-(6-(1- | d₆) δ (ppm) 8.82 (s, 1H,) 8.22 |
| | methyl-1H-pyrazol-4-yl)pyrazolo[1,5- | (s, 1H), 7.97 (s, 1H), 7.83- |
| | a]pyridin-3-yl)piperazine-1-carboxylate | 7.76 (m, 1H), 7.77 (s, 1H), |
| | | 7.65 (d, J = 9.2 Hz, 1H), 7.54 |
| | | (d, J = 12.0 Hz, 1H), 7.43 (d, |
| | | J = 7.6 Hz, 1H), 7.33 (d, J = |
| | | 9.2 Hz, 1H), 5.81 (q, J = 6.4 |
| | | Hz, 1H), 3.87 (s, 3H), 3.78- |
| | | 3.46 (m, 4H), 3.07-2.90 (m, |
| | | 4H), 1.50 (d, J = 6.4 Hz, 3H). |
| | | MS (ESI) m/z [M + H]⁺ calcd. |
| | | for C₂₆H₂₄F₄N₆O₂: 517.2, |
| | | Found: 517.3; HPLC purity: |
| | | 210 nm: 100.0%; 254 nm: |
| | | 100.0%. |

### Example 205: Synthesis of Exemplary Compound 274

### N-[(1R)-1-(4-chlorophenyl)ethyl]-4-[6-(1-methyl-1H-pyrazol-4-yl)pyrazolo[1,5-a]pyridin-3-yl]piperazine-1-carboxamide (Compound 274)

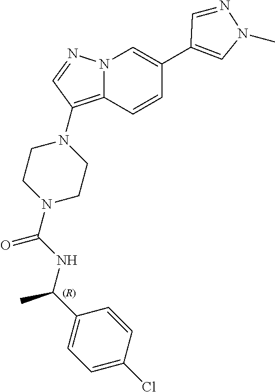

To a solution of 1-chloro-4-[(1R)-1-isocyanatoethyl]benzene (85 mg, 0.47 mmol) in ACN (6 mL) and DIPEA (0.41 mL, 2.35 mmol) was added 6-(1-methyl-1H-pyrazol-4-yl)-3-piperazin-1-ylpyrazolo[1,5-a]pyridine hydrochloride (Compound S19) (150 mg, 0.470 mmol). The reaction was stirred at rt for 1.5 h, then the solvent was concentrated under reduced pressure. The residue was purified by silica gel chromatography eluting with a gradient of acetone (0.1% Et₃N additive)/EtOAc to give a solid. The crude product was purified by C18 reverse-phase chromatography eluting with a gradient of water/ACN to give 111 mg (51%) of N-[(1R)-1-(4-chlorophenyl)ethyl]-4-[6-(1-methyl-1H-pyrazol-4-yl)pyrazolo[1,5-a]pyridin-3-yl]piperazine-1-carboxamide (Compound 274) as a solid. ¹H NMR (400 MHZ, DMSO-d₆) δ 8.81 (s, 1H), 8.22 (s, 1H), 7.96 (s, 1H), 7.77 (s, 1H), 7.65 (m, 1H), 7.36 (m, 4H), 7.31 (dd, J=9.3, 1.5 Hz, 1H), 6.90 (d, J=7.7 Hz, 1H), 4.84 (q, J=7.2 Hz, 1H), 3.87 (s, 3H), 3.51 (m, 4H), 2.94 (m, 4H), 1.36 (d, J=7.1, 3H). MS (ESI) m/z [M+H]⁺ calcd. for C₂₄H₂₇ClN₇O: 464.2. Found: 464.2; HPLC purity: 210 nm: 100.0%; 254 nm: 100.0%.

Using the procedures described in Example 205, Compound 274 and reagents, starting materials, and conditions known to those skilled in the art were used to prepare the compounds shown in Table II.

TABLE II

| Compound | Name | Characterization |
|---|---|---|
| Compound | N-(4-chlorobenzyl)-4-[6-(1-methyl-1H- | ¹H NMR (400 MHz, DMSO- |
| 275 | pyrazol-4-yl)pyrazolo[1,5-a]pyridin-3- | d₆) δ (ppm) 8.81 (s, 1H), 8.22 |
| | yl]piperazine-1-carboxamide | (s, 1H), 7.96 (s, 1H), 7.77 (s, |
| | | 1H), 7.66 (d, J = 8.7 Hz, 1H), |
| | | 7.37 (m, 2H), 7.31 (m, 3H) |
| | | 7.21 (t, J = 5.9 Hz, 1H), 4.25 |
| | | (d, J = 5.7 Hz, 2H), 3.87 (s, |
| | | 3H), 3.52 (m, 4H), 2.95 (m, |
| | | 4H). MS (ESI) m/z [M + H]⁺ |
| | | calcd. for C₂₃H₂₄ClN₇O: |
| | | 450.2. Found: 450.2; HPLC |
| | | purity: 210 nm: 98.7%; 254 |
| | | nm: 98.3%. |

### Example 206: Synthesis of Exemplary Compound 276

### (R)—N-(1-(4-chlorophenyl)ethyl)-N-methyl-4-(6-(1-methyl-1H-pyrazol-4-yl)pyrazolo[1,5-a]pyridin-3-yl)piperazine-1-carboxamide (Compound 276)

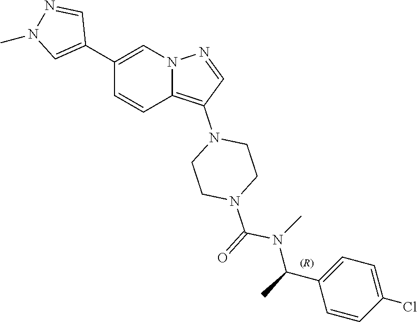

To a vial containing triphosgene (0.0602 g, 0.203 mmol) cooled to 0° C. was added a solution of (1R)-1-(4-chlorophenyl)-N-methylethanamine (0.115 g, 0.676 mmol) and Et₃N (0.0684 g, 0.676 mmol) in DCM (3 mL) dropwise over 10 min. The mixture was stirred for 5 min at 0° C. then Et₃N (0.0684 g, 0.676 mmol) was added. The reaction was stirred at 0° C. for 30 min, warmed to rt, and stirred for an additional 30 min. The mixture was cooled to 0° C., Et₃N (0.428 mL, 3.07 mmol), 6-(1-methyl-1H-pyrazol-4-yl)-3-piperazin-1-ylpyrazolo[1,5-a]pyridine hydrochloride (Compound S19) (0.196 g, 0.615 mmol), and DMAP (2 mg, 0.01 mmol) were added sequentially. The mixture was warmed to rt and stirred overnight. The reaction was diluted with DCM (25 mL) and washed with sat. aq. NaHCO₃ (20 mL), and water (20 mL), then dried over anhydrous Na₂SO₄ and filtered. The filtrate was concentrated under reduced pressure then the residue was purified by flash chromatography eluting with 0-10% MeOH in DCM. The product was treated with 25% Et₂O/hexanes and concentrated under reduced pressure. This was repeated two more times to give a solid. The solid was stirred with 25% Et₂O/hexanes for 30 min and filtered. The solid was dried to afford 63.4 mg (22%) of (R)—N-(1-(4-chlorophenyl)ethyl)-N-methyl-4-(6-(1-methyl-1H-pyrazol-4-yl)pyrazolo[1,5-a]pyridin-3-yl)piperazine-1-carboxamide (Compound 276) as a yellow solid. ¹H NMR (400 MHz, CDCl₃) δ (ppm) 8.47 (s, 1H), 7.75 (s, 1H), 7.74 (br s, 1H), 7.61 (s, 1H), 7.54 (br s, 1H), 7.35-7.25 (m, 4H), 7.16 (d, J=8.8 Hz, 1H), 5.30 (q, J=7.2 Hz, 1H), 3.98 (s, 3H), 3.60-3.40 (m, 4H), 3.22-3.10 (m, 4H), 2.64 (s, 3H), 1.56 (d, J=7.2 Hz, 3H). MS (ESI) m/z [M+H]⁺ calcd. for C₂₅H₂₈ClN₇O: 478.2, Found: 478.2; HPLC purity: 210 nm: 100.0%; 254 nm: 100.0%.

Using the procedures described in Example 206, Compound 276 and reagents, starting materials, and conditions known to those skilled in the art were used to prepare the compounds shown in Table JJ.

TABLE JJ

| Compound | Name | Characterization |
|---|---|---|
| Compound | 3-(4-{[(2R)-2-(4-chlorophenyl)pyrrolidin- | ¹H NMR (400 MHz, CDCl₃) δ |
| 277 | 1-yl]carbonyl}piperazin-1-yl)-6-(1- | 8.46 (s, 1H), 7.74 (s, 1H) 7.70 |
| | methyl-1H-pyrazol-4-yl)pyrazolo[1,5- | (s, 1H) 7.61 (s, 1H), 7.50 (d, J = |
| | a]pyridine | 8.9 Hz, 1H), 7.28 (m, 2H), |
| | | 7.22 (m, 2H) 7.14 (d, J = 9 Hz, |
| | | 1H), 5.04 (dd, J = 9, 7 Hz, |
| | | 1H), 3.97 (s, 3H), 3.65 (m, |
| | | 2H), 3.58 (br s 2H) 3.46 (br s, |
| | | 2H) 3.08 (br s, 2H) 2.99 (br s, |
| | | 2H) 2.37 (m, 1H), 1.99 (m, |
| | | 1H), 1.90 (m, 1H), 1.74 (m, |
| | | 1H). MS (ESI) m/z [M + H]⁺ |
| | | calcd. for C₂₆H₂₈ClN₇O: |
| | | 490.2. Found: 490.2; HPLC |
| | | purity: 210 nm: 100.0%; 254 |
| | | nm: 100.0%. |
| Compound | N-(4-chlorobenzyl)-N-methyl-4-[6-(1- | ¹H NMR (400 MHz, CDCl₃) δ |
| 278 | methyl-1H-pyrazol-4-yl)pyrazolo[1,5- | (ppm) 8.45 (s, 1H), 7.72-7.77 |
| | a]pyridin-3-yl]piperazine-1-carboxamide | (m, 1H), 7.70 (s, 1H), 7.60 (s, |
| | | 1H), 7.50 (d, J = 9.17 Hz, 1H), |
| | | 7.28-7.36 (m, 2H), 7.19- |
| | | 7.27 (m, 2H), 7.13 (dd, |
| | | J = 9.17, 1.34 Hz, 1H), 4.40 (s, |
| | | 2H), 3.95 (s, 3H), 3.41-3.54 |
| | | (m, 4H), 3.02-3.15 (m, 4H), |
| | | 2.80 (s, 3H). MS (ESI) m/z |
| | | [M + H]⁺ calcd. for |
| | | C₂₄H₂₆ClN₇O, 464.2; found, |
| | | 464.1; HPLC purity: 210 nm: |
| | | 100.0%; 254 nm: 100.0%. |
| Compound | 4-[6-(1-methyl-1H-pyrazol-4- | ¹H NMR (400 MHz, DMSO- |
| 279 | yl)pyrazolo[1,5-a]pyridin-3-yl]-N-[4- | d₆) δ 8.82 (s, 1H), 8.22 (s, 1H), |
| | (trifluoromethyl)benzyl]piperazine-1- | 7.97 (s, 1H), 7.77 (s, 1H), 7.67 |
| | carboxamide | (m, 3H), 7.50 (d, J = 7.9 Hz, |
| | | 2H), 7.31 (m, 2H), 4.34 (d, J = |
| | | 5.5 Hz, 2H), 3.87 (s, 3H), 3.54 |
| | | (m, 4H), 2.96 (m, 4H). MS |
| | | (ESI) m/z [M + H]⁺ calcd. for |
| | | C₂₄H₂₄F₃N₇O: 484.2. Found: |
| | | 484.2; HPLC purity: 210 nm: |
| | | 98.65%; 254 nm: 100.0%. |

### Example 207: Synthesis of Exemplary Compound 280

### 2-carbamoylbenzyl 4-(6-(1-methyl-1H-pyrazol-4-yl)pyrazolo[1,5-a]pyridin-3-yl)piperazine-1-carboxylate (Compound 280)

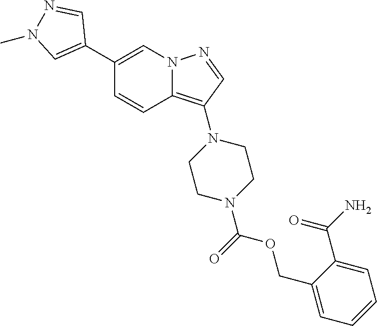 [SMILES: Cn1cc(-c2ccc3c(N4CCN(C(=O)OCc5ccccc5C(N)=O)CC4)cnn3c2)cn1]

### Step 1. Preparation of 2-carbamoylbenzyl (4-nitrophenyl)carbonate

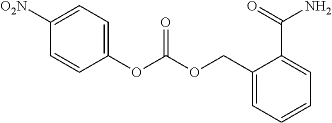 [SMILES: NC(=O)c1ccccc1COC(=O)Oc1ccc([N+](=O)[O-])cc1]

To a solution of 2-(hydroxymethyl) benzamide (225.0 mg, 1.488 mmol) and DCM (15 mL) at 4° C. under nitrogen was added pyridine (0.5056 mL, 6.252 mmol). The mixture was stirred for 10 min and 4-nitrophenyl chloroformate (0.6000 g, 2.977 mmol) was added as a solid in one portion at 4° C. After 10 min the reaction was allowed to warm to rt and stirred at rt overnight. The solvent was removed under reduced pressure and then purified by chromatography eluting with a gradient of 0-60% DCM in acetone with 0.1% Et₃N to afford 0.273 g (58%) of 2-carbamoylbenzyl (4-nitrophenyl)carbonate as a white solid. ¹H NMR (400 MHz, CDCl₃) δ (ppm) 8.28 (m, 2H), 7.61 (m, 2H), 7.54 (td, J=7.5, 1.3 Hz, 1H), 7.46 (m, 1H), 7.42 (m, 2H), 6.10 (br s, 1H), 5.81 (br s, 1H), 5.62 (s, 2H). MS (ESI) m/z [M+H]⁺ calcd. for C₁₅H₁₂N₂O₆: 317.1, Found: 317.1.

### Step 2. Preparation of 2-carbamoylbenzyl 4-(6-(1-methyl-1H-pyrazol-4-yl)pyrazolo[1,5-a]pyridin-3-yl)piperazine-1-carboxylate (Compound 280)

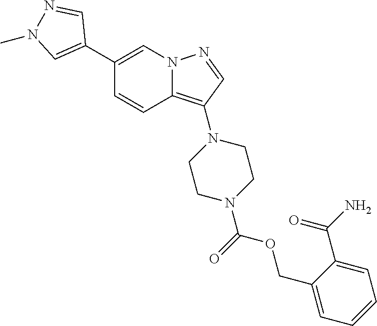 [SMILES: Cn1cc(-c2ccc3c(N4CCN(C(=O)OCc5ccccc5C(N)=O)CC4)cnn3c2)cn1]

A mixture of 2-carbamoylbenzyl 4-nitrophenyl carbonate (265 mg, 0.838 mmol, as prepared in the previous step) and 6-(1-methyl-1H-pyrazol-4-yl)-3-piperazin-1-ylpyrazolo[1,5-a]pyridine (Compound 12) (284 mg, 1.00 mmol) in ACN (13.2 mL) was sonicated stirred at rt for 2 h. The mixture was partitioned between DCM and aq. Na₂CO₃. The layers were filtered through a frit, and the solid rinsed with more water and DCM, then dried in a vacuum oven at 50° C. for 15 h to afford 300 mg (78%) of 2-carbamoylbenzyl 4-(6-(1-methyl-1H-pyrazol-4-yl)pyrazolo[1,5-a]pyridin-3-yl)piperazine-1-carboxylate (Compound 280) as a white solid. ¹H NMR (400 MHZ, DMSO-d₆) δ (ppm) 8.82 (s, 1H), 8.22 (s, 1H), 7.96 (s, 1H), 7.85 (br s, 1H), 7.77 (s, 1H), 7.65 (d, J=9.2 Hz, 1H), 7.46 (m, 4H), 7.38 (m, 1H), 7.33 (dd, J=9.2, 1.3 Hz, 1H), 5.30 (s, 2H), 3.87 (s, 3H), 3.60 (br s, 4H), 2.98 (m, 4H). MS (ESI) m/z [M+H]⁺ calcd. For C₂₄H₂₅N₇O₃: 460.2, Found: 460.3; HPLC purity: 210 nm: 100.0%; 254 nm: 100.0%.

### Example 208: Synthesis of Exemplary Compound 281

### 2-cyanobenzyl 4-(6-(1-methyl-1H-pyrazol-4-yl)pyrazolo[1,5-a]pyridin-3-yl)piperazine-1-carboxylate (Compound 281)

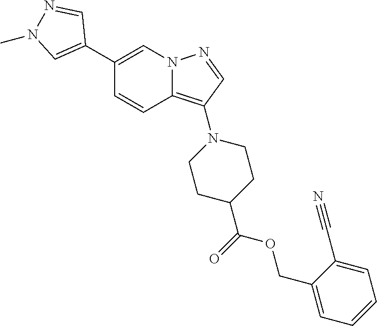 [SMILES: Cn1cc(-c2ccc3c(N4CCC(C(=O)OCc5ccccc5C#N)CC4)cnn3c2)cn1]

A solution of 2-carbamoylbenzyl 4-[6-(1-methyl-1H-pyrazol-4-yl)pyrazolo[1,5-a]pyridin-3-yl]piperazine-1-carboxylate (Compound 280) (151 mg, 0.328 mmol) in anhydrous THF (6 mL) was stirred under nitrogen then (methoxycarbonylsulfamoyl)triethylammonium hydroxide, inner salt (Burgess reagent) (156 mg, 0.656 mmol) was added in one dose. The reaction was stirred at rt for 1 h then ACN (6.00 mL) was added. Additional Burgess reagent (156 mg, 0.656 mmol) was added, and the reaction was stirred at rt for 30 min. The reaction was concentrated under reduced pressure then the residue was purified by silica gel column chromatography eluting with a gradient of 5-60% DCM in acetone with 0.1% Et₃N to afford 132 mg (91%) of 2-cyanobenzyl 4-(6-(1-methyl-1H-pyrazol-4-yl)pyrazolo[1,5-a]pyridin-3-yl)piperazine-1-carboxylate (Compound 281) as a light yellow solid. ¹H NMR (400 MHZ, DMSO-d₆) δ (ppm) 8.82 (s, 1H), 8.22 (s, 1H), 7.96 (s, 1H), 7.90 (dd, J=7.7, 0.9 Hz, 1H), 7.75 (m, 2H), 7.65 (d, J=8.9 Hz, 2H), 7.56 (td, J=7.6, 1.0 Hz, 1H), 7.32 (dd, J=9.2, 1.4 Hz, 1H), 5.27 (s, 2H), 3.86 (s, 3H), 3.60 (br s, 4H), 2.98 (m, 4H). MS (ESI) m/z [M+H]⁺ calcd. for C₂₄H₂₃N₇O₂: 442.2, Found: 442.3; HPLC purity: 210 nm: 100.0%; 254 nm: 100.0%.

### Example 209: Synthesis of Exemplary Compound 282

### (R)-1-(4-chlorophenyl)ethyl 4-(7-(1-methyl-1H-pyrazol-4-yl)imidazo[1,2-b]pyridazin-3-yl)piperazine-1-carboxylate (Compound 282)

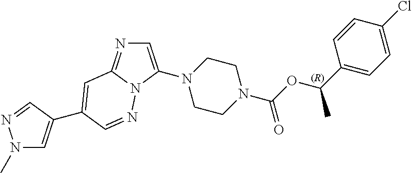

### Step 1. Preparation of (R)-1-(4-chlorophenyl)ethyl (4-nitrophenyl)carbonate

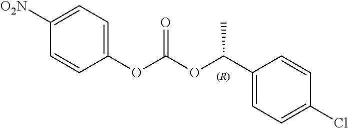

A mixture of (1R)-1-(4-chlorophenyl)ethanol (0.52 g, 3.3 mmol) and DCM (34 mL) was cooled to 0° C. Pyridine (1.13 mL, 13.9 mmol) was added, the reaction was stirred for 10 min at 0° C., then 4-nitrophenyl chloroformate (1.34 g, 6.64 mmol) was added in one portion. The mixture was warmed to rt and stirred overnight. The reaction was concentrated under reduced pressure then the residue was triturated with 50% EtOAc/hexanes (3×15 mL) and filtered. The combined filtrates were concentrated under reduced pressure then the residue was purified by flash chromatography eluting with 0-50% DCM/hexanes to afford 737.4 mg (69%) of (R)-1-(4-chlorophenyl)ethyl (4-nitrophenyl)carbonate as a white solid. ¹H NMR (400 MHZ, CDCl₃) δ (ppm) 8.27 (d, J=9.2 Hz, 2H), 7.38 (s, 4H), 7.36 (d, J=9.2 Hz, 2H), 5.82 (q, J=6.8 Hz, 1H), 1.69 (d, J=6.8 Hz, 3H). HPLC purity: 210 nm: 100.0%; 254 nm: 100.0%.

### Step 2. Preparation of (R)-1-(4-chlorophenyl)ethyl 4-(7-(1-methyl-1H-pyrazol-4-yl)imidazo[1,2-b]pyridazin-3-yl)piperazine-1-carboxylate (Compound 282)

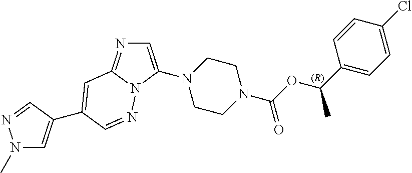

To a suspension of (1R)-1-(4-chlorophenyl)ethyl 4-nitrophenyl carbonate (0.3697 g, 1.149 mmol, as prepared in the previous step), 7-(1-methyl-1H-pyrazol-4-yl)-3-piperazin-1-ylimidazo[1,2-b]pyridazine hydrochloride (Compound S83) (0.210 g, 0.657 mmol), and ACN (9.26 mL) was added DIPEA (3.317 mL, 19.04 mmol), then the mixture was stirred at rt for 18 h. The reaction was concentrated under reduced pressure, then the residue was purified by flash chromatography eluting with 0-10% IPA/DCM. The crude product was treated with hexanes and concentrated under reduced pressure several times then the solid was treated with 25% Et₂O/hexanes and concentrated under reduced pressure several times to afford 183.2 mg (60%) of (R)-1-(4-chlorophenyl)ethyl 4-(7-(1-methyl-1H-pyrazol-4-yl)imidazo[1,2-b]pyridazin-3-yl)piperazine-1-carboxylate (Compound 282) as a yellow solid. ¹H NMR (400 MHZ, CDCl₃) δ (ppm) 8.54 (d, J=2.0 Hz, 1H), 7.97 (d, J=2.0 Hz, 1H), 7.86 (s, 1H), 7.77 (s, 1H), 7.39-7.29 (m, 4H), 7.25 (s, 1H), 5.83 (q, J=6.4 Hz, 1H), 4.01 (s, 3H), 3.83-3.70 (m, 4H), 3.33-3.23 (m, 4H), 1.57 (d, J=6.4 Hz, 3H). MS (ESI) m/z [M+H]⁺ calcd. for C₂₃H₂₄ClN₇O₂: 466.2, Found: 466.2; HPLC purity: 210 nm: 97.8%; 254 nm: 99.2%.

Using the procedures described in Example 209, Compound 282 and reagents, starting materials, and conditions known to those skilled in the art were used to prepare the compounds shown in Table KK.

TABLE KK

| Compound | Name | Characterization |
|---|---|---|
| Compound | (R)-1-(4-(trifluoromethyl)phenyl)ethyl 4- | ¹H NMR (400 MHz, DMSO- |
| 283 | (7-(1-methyl-1H-pyrazol-4- | d₆) δ (ppm) 8.84 (d, J = 2.4 |
| | yl)imidazo[1,2-b]pyridazin-3- | Hz, 1H), 8.37 (s, 1H), 8.17 (d, |
| | yl)piperazine-1-carboxylate | J = 2.4 Hz, 1H), 8.11 (s, 1H), |
| | | 7.74 (d, J = 8.0 Hz, 2H), 7.61 |
| | | (d, J = 8.0 Hz, 2H), 7.30 (s, |
| | | 1H), 5.83 (q, J = 6.4 Hz, 1H), |
| | | 3.89 (s, 3H), 3.82-3.48 (m, |
| | | 4H), 3.25-3.08 (m, 4H), 1.51 |
| | | (d, J = 6.4 Hz, 3H). MS (ESI) |
| | | m/z [M + H]⁺ calcd. for |
| | | C₂₄H₂₄F₃N₇O₂: 500.2, Found: |
| | | 500.3; HPLC purity: 210 nm: |
| | | 99.4%; 254 nm: 98.5%. |

### Example 210: Synthesis of Exemplary Compound 284

### 2,2,2-trifluoro-1-(2-(4-(6-(1-methyl-1H-pyrazol-4-yl)pyrazolo[1,5-a]pyridin-3-yl)piperazin-1-yl)pyrimidin-5-yl)ethan-1-one (Compound 284)

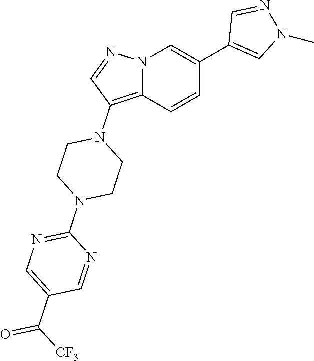 [SMILES: Cn1cc(-c2ccc3c(N4CCN(c5ncc(C(=O)C(F)(F)F)cn5)CC4)cnn3c2)cn1]

### Step 1. Preparation of 1-(2-chloropyrimidin-5-yl)-2,2,2-trifluoroethanone

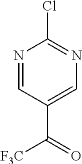 [SMILES: O=C(c1cnc(Cl)nc1)C(F)(F)F]

To a solution of 2-chloro-5-iodopyrimidine (1.00 g, 4.16 mmol) in THF (10 mL) cooled to −40° C. was slowly added 1.3 M of iPrMgCl·LiCl in THF (3.68 mL, 4.78 mmol) at less than −30° C. The mixture stirred for 30 min, then a solution of 2,2,2-trifluoro-N-methoxy-N-methylacetamide (0.686 g, 4.37 mmol) in THF (1 mL) was added. After 1 h at −40° C., the reaction was warmed to 0° C. After 1 h, the reaction was quenched by the addition of sat. aq. NH₄Cl. The pH was adjusted to approximately 8 with sat. aq. NaHCO₃ and the mixture was extracted with EtOAc (2×). The combined extracts were washed with brine, dried over anhydrous Na₂SO₄, and filtered. The filtrate was concentrated under reduced pressure. The solid was triturated with DCM to provide 0.59 g (41%) of 1-(2-chloropyrimidin-5-yl)-2,2,2-trifluoroethanone as a yellow solid. ¹H NMR (400 MHZ, CD₃OD) δ (ppm) 8.89 (s, 2H). MS (ESI) m/z [M+H]⁺ calcd. for C₆H3ClF₃N₂O, 211.0; found, 211.0.

### Step 2. Preparation of 2,2,2-trifluoro-1-(2-(4-(6-(1-methyl-1H-pyrazol-4-yl)pyrazolo[1,5-a]pyridin-3-yl)piperazin-1-yl)pyrimidin-5-yl)ethan-1-one (Compound 284)

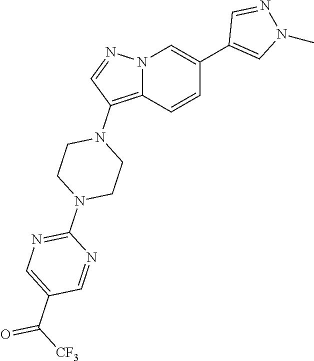 [SMILES: Cn1cc(-c2ccc3c(N4CCN(c5ncc(C(=O)C(F)(F)F)cn5)CC4)cnn3c2)cn1]

To a suspension of 1-(2-chloropyrimidin-5-yl)-2,2,2-trifluoroethanone (288 mg, 1.37 mmol, as prepared in the previous step) and 6-(1-methyl-1H-pyrazol-4-yl)-3-piperazin-1-ylpyrazolo[1,5-a]pyridine hydrochloride (436 mg, 1.12 mmol) in NMP (10 mL) was added K₂CO₃ (473 mg, 3.42 mmol). The reaction stirred at 110° C. for 4 h then rt overnight. The mixture was added to water, stirred, and the resulting solids were collected by suction filtration, washed with H₂O, and dried under reduced pressure. The solids were purified by silica gel chromatography eluting with a gradient of 25-30% acetone/DCM to afford 260 mg (51%) of 2,2,2-trifluoro-1-(2-(4-(6-(1-methyl-1H-pyrazol-4-yl)pyrazolo[1,5-a]pyridin-3-yl)piperazin-1-yl)pyrimidin-5-yl)ethan-1-one (Compound 284) as a yellow solid. ¹H NMR (400 MHZ, CDCl₃) δ (ppm) 8.90-9.04 (m, 2H), 8.50 (s, 1H), 7.76-7.80 (m, 1H), 7.75 (s, 1H), 7.64 (s, 1H), 7.57 (d, J=9.17 Hz, 1H), 7.28 (s, 2H), 7.20 (dd, J=9.17, 1.47 Hz, 1H), 4.22-4.30 (m, 4H), 4.00 (s, 3H), 3.13-3.23 (m, 4H). MS (ESI) m/z [M+H]⁺ calcd. for C₂₁H₂₀F₃N₈O, 457.2; found, 457.2; HPLC purity (sum of 2 peaks corresponding to hydrate and ketone): 210 nm: 100.0%; 254 nm: 100.0%.

### Example 211: Synthesis of Exemplary Compound 285

### 2,2,2-trifluoro-1-(2-{4-[6-(1-methyl-1H-pyrazol-4-yl)pyrazolo[1,5-a]pyridin-3-yl]piperazin-1-yl}pyrimidin-5-yl)ethanol (Compound 285)

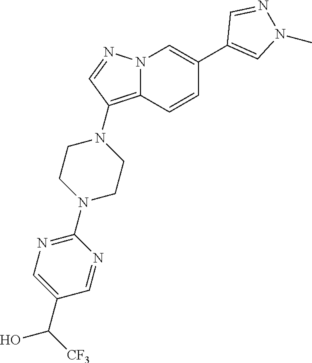 [SMILES: Cn1cc(-c2ccc3c(N4CCN(c5ncc(C(O)C(F)(F)F)cn5)CC4)cnn3c2)cn1]

To a suspension of 2,2,2-trifluoro-1-(2-{4-[6-(1-methyl-1H-pyrazol-4-yl)pyrazolo[1,5-a]pyridin-3-yl]piperazin-1-yl}pyrimidin-5-yl)ethenone (Compound 284) (200 mg, 0.4 mmol) in MeOH (5 mL) and THF (5 mL) was added NaBH₄ (58.0 mg, 1.53 mmol) in one portion. The reaction was allowed to stir at rt overnight and then partitioned between sat. aq. NaHCO₃ and EtOAc. The organic phase was washed with brine, dried over anhydrous Na₂SO₄, and filtered. The filtrate was concentrated under reduced pressure. The residue was purified by silica gel chromatography eluting with 50% acetone/DCM to afford 151 mg (80%) of 2,2,2-trifluoro-1-(2-{4-[6-(1-methyl-1H-pyrazol-4-yl)pyrazolo[1,5-a]pyridin-3-yl]piperazin-1-yl}pyrimidin-5-yl)ethanol (Compound 285) as a white solid. ¹H NMR (400 MHz, DMSO-d₆) δ (ppm) 8.83 (s, 1H), 8.46 (s, 2H), 8.23 (s, 1H), 7.98 (s, 1H), 7.79 (s, 1H), 7.70 (d, J=9.17 Hz, 1H), 7.34 (dd, J=9.23, 1.41 Hz, 1H), 6.88 (d, J=5.14 Hz, 1H), 5.05-5.20 (m, 1H), 3.93-4.01 (m, 4H), 3.88 (s, 3H), 3.01-3.12 (m, 4H). MS (ESI) m/z [M+H]⁺ calcd. for C₂₁H₂₂F₃N₈O, 459.2; found, 457.2; HPLC purity: 210 nm: 100.0%; 254 nm: 100.0%.

### Example 212: Synthesis of Exemplary Compound 286

### 1-(2-(4-(6-(1-methyl-1H-pyrazol-4-yl)pyrazolo[1,5-a]pyridin-3-yl)piperazin-1-yl)pyrimidin-5-yl)propane-1,2-diol (Compound 286)

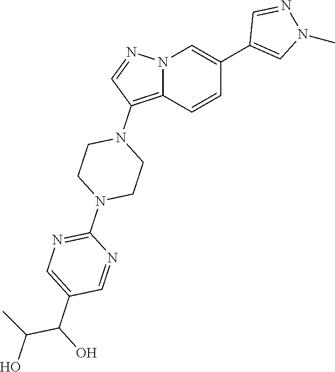 [SMILES: CC(O)C(O)c1cnc(N2CCN(c3cnn4cc(-c5cnn(C)c5)ccc34)CC2)nc1]

### Step 1. Preparation of (E)-2-chloro-5-(prop-1-en-1-yl)pyrimidine

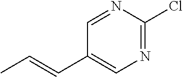

A mixture of 5-bromo-2-chloropyrimidine (0.901 g, 4.66 mmol), (1E)-prop-1-en-1-ylboronic acid (0.200 g, 2.33 mmol), Pd(dppf)Cl₂·DCM (95.1 mg, 0.116 mmol), and K₂CO₃ (0.965 g, 6.98 mmol) in toluene (8.0 mL) and water (0.20 mL) was sparged for 5 min with argon then the mixture was heated to 90° C., and stirred under nitrogen for 5 h. The mixture was cooled to rt and diluted with EtOAc (40 mL) and H₂O (40 mL). The phases were separated, and the aqueous phase was extracted with EtOAc (20 mL). The organic phases were combined, dried over anhydrous MgSO₄, and filtered. The filtrate was concentrated under reduced pressure. The crude material was purified by flash chromatography eluting with 5-10% EtOAc/hexanes to afford 203 mg (56%) of (E)-2-chloro-5-(prop-1-en-1-yl)pyrimidine as a light gray-solid. ¹H NMR (400 MHZ, CDCl₃) δ (ppm) 8.56 (s, 2H), 6.43 (dq, J=16.0, 6.4 Hz, 1H), 6.31 (dd, J=16.0, 1.6 Hz, 1H), 1.96 (dd, J=6.4, 1.6 Hz, 3H).

### Step 2. Preparation of (E)-6-(1-methyl-1H-pyrazol-4-yl)-3-(4-(5-(prop-1-en-1-yl)pyrimidin-2-yl)piperazin-1-yl)pyrazolo[1,5-a]pyridine

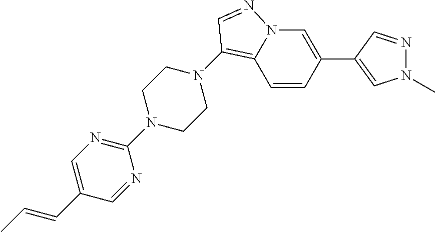 [SMILES: C/C=C/c1cnc(N2CCN(c3cnn4cc(-c5cnn(C)c5)ccc34)CC2)nc1]

A solution of (E)-2-chloro-5-(prop-1-en-1-yl)pyrimidine (201 mg, 1.30 mmol, as prepared in the previous step) and 6-(1-methyl-1H-pyrazol-4-yl)-3-piperazin-1-ylpyrazolo[1,5-a]pyridine hydrochloride (Compound S19) (518 mg, 1.30 mmol) in NMP (8.3 mL) was treated with K₂CO₃ (494 mg, 3.58 mmol) and the reaction was stirred at 110° C. for 5.5 h, then cooled to rt. The mixture was diluted with EtOAc (15 mL). The precipitate was filtered, washed with water (2×15 mL) and then dried under reduced pressure to afford 435 mg (84%) of (E)-6-(1-methyl-1H-pyrazol-4-yl)-3-(4-(5-(prop-1-en-1-yl)pyrimidin-2-yl)piperazin-1-yl)pyrazolo[1,5-a]pyridine as a light tan solid. ¹H NMR (400 MHZ, CDCl₃) δ (ppm) 8.47 (s, 1H), 8.34 (s, 2H), 7.75 (s, 1H), 7.72 (s, 1H), 7.62 (s, 1H), 7.56 (d, J=8.8 Hz, 1H), 7.15 (dd, J=8.8, 1.4 Hz, 1H), 6.21 (d, J=16.8 Hz, 1H), 6.10 (dq, J=15.6, 6.4 Hz, 1H), 4.00-4.08 (m, 4H), 3.98 (s, 3H), 3.07-3.22 (m, 4H), 1.88 (dd, J=6.4, 1.2 Hz, 3H). MS (ESI) m/z [M+H]⁺ calcd. for C₂₂H₂₄N₈: 401.2, Found: 401.2.

### Step 3. Preparation of 1-(2-(4-(6-(1-methyl-1H-pyrazol-4-yl)pyrazolo[1,5-a]pyridin-3-yl)piperazin-1-yl)pyrimidin-5-yl)propane-1,2-diol (Compound 286)

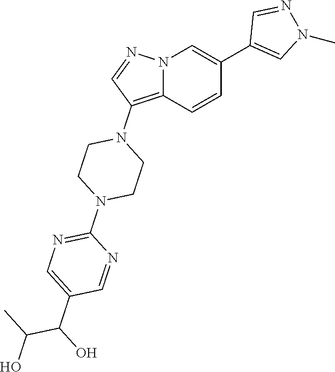 [SMILES: CC(O)C(O)c1cnc(N2CCN(c3cnn4cc(-c5cnn(C)c5)ccc34)CC2)nc1]

A mixture of (E)-6-(1-methyl-1H-pyrazol-4-yl)-3-(4-(5-(prop-1-en-1-yl)pyrimidin-2-yl)piperazin-1-yl)pyrazolo[1,5-a]pyridine (250 mg, 0.624 mmol, as prepared in the previous step) in dioxane (9.0 mL) was treated with K₂[OsO₂(OH)₄] (18.4 mg, 0.0499 mmol) and NMO (117 mg, 0.999 mmol). The mixture was stirred at 50° C. for 21 h then cooled to rt and concentrated under reduced pressure. The residue was purified by flash chromatography eluting with 10-20% EtOH/DCM. The material was triturated with ACN (5 mL) then the solids were isolated by filtration, the filter cake was washed with ACN, and the solid was dried by suction to afford 169 mg (62%) of 1-(2-(4-(6-(1-methyl-1H-pyrazol-4-yl)pyrazolo[1,5-a]pyridin-3-yl)piperazin-1-yl)pyrimidin-5-yl)propane-1,2-diol (Compound 286) as a gray solid. ¹H NMR (400 MHZ, DMSO-d₆) δ (ppm) 8.82 (s, 1H), 8.30 (s, 2H), 8.22 (s, 1H), 7.97 (s, 1H), 7.78 (s, 1H), 7.69 (d, J=9.2 Hz, 1H), 7.33 (dd, J=9.2, 1.2 Hz, 1H), 5.20 (d, J=4.4 Hz, 1H), 4.65 (d, J=4.8 Hz, 1H), 4.29 (t, J=4.8 Hz, 1H), 3.89-3.97 (m, 4H), 3.87 (s, 3H), 3.63-3.78 (m, 1H), 3.07-3.03 (m, 4H), 0.90 (d, J=6.4 Hz, 3H). MS (ESI) m/z [M+H]⁺ calcd. for C₂₂H₂₆N₈O₂: 435.2, Found: 435.3; HPLC purity: 210 nm: 98.4%; 254 nm: 97.5%.

### Example 213: Synthesis of Exemplary Compound 287

### 1-(2-{4-[6-(1-methyl-1H-pyrazol-4-yl)pyrazolo[1,5-a]pyridin-3-yl]piperazin-1-yl}pyrimidin-5-yl)propan-1-ol (Compound 287)

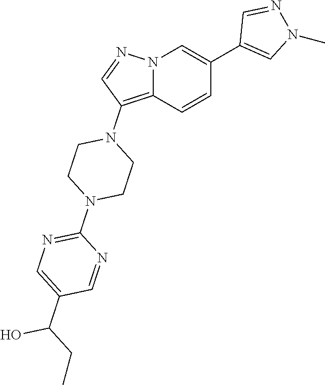 [SMILES: CCC(O)c1cnc(N2CCN(c3cnn4cc(-c5cnn(C)c5)ccc34)CC2)nc1]

### Step 1. Preparation of 1-(2-chloropyrimidin-5-yl)propan-1-one

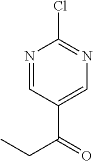

To a cooled (−30° C. to −40° C.) solution of 2-chloro-5-iodopyrimidine (4.02 g, 16.7 mmol) in THF (40 mL) was slowly added 1.3M iPrMgCl· LiCl in THF (16.74 mL, 21.77 mmol) dropwise at less than −30° C. After 30 mins a solution of N-methoxy-N-methylpropanamide (2.55 g, 21.8 mmol) in THF (3 ml) was added slowly, at less than −25° C. After 1 h, the reaction was warmed to approximately 0° C. After 1 h, the reaction was allowed to warm to rt and quenched with sat. aq. NH₄Cl. The pH was adjusted to approximately 8 with sat. aq. NaHCO₃ then partitioned between EtOAc and water. The aqueous phase was extracted twice with EtOAc, and the combined organic phases washed with brine, dried over anhydrous Na₂SO₄, filtered, and concentrated under reduced pressure. The residue was purified by silica gel chromatography eluting with a gradient of 0-20% acetone/DCM to afford 1.04 g (36%) of 1-(2-chloropyrimidin-5-yl)propan-1-one as a salmon-colored semi-solid. ¹H NMR (400 MHZ, CDCl₃) δ (ppm) 9.11 (s, 2H), 8.66 (d, J=4.77 Hz, 4H), 7.22-7.37 (m, 2H), 3.01 (q, J=7.21 Hz, 2H), 1.26 (t, J=7.15 Hz, 3H). MS (ESI) m/z [M+H]⁺ calcd. for C₇H₈ClN₂O, 171.0; found, 171.0.

### Step 2. Preparation of 1-(2-{4-[6-(1-methyl-1H-pyrazol-4-yl)pyrazolo[1,5-a]pyridin-3-yl]piperazin-1-yl}pyrimidin-5-yl)propan-1-ol

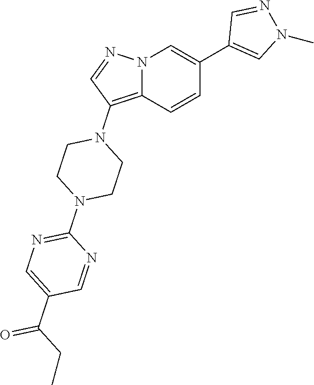 [SMILES: CCC(=O)c1cnc(N2CCN(c3cnn4cc(-c5cnn(C)c5)ccc34)CC2)nc1]

To a suspension of 1-(2-chloropyrimidin-5-yl)propan-1-one (256 mg, 1.50 mmol, as prepared in the previous step) and 6-(1-methyl-1H-pyrazol-4-yl)-3-piperazin-1-ylpyrazolo[1,5-a]pyridine hydrochloride (Compound S19) (613 mg, 1.35 mmol) in NMP (10 mL) was added K₂CO₃ (518 mg, 3.75 mmol). The mixture was heated at 110° C. for 3 h, allowed to cool to rt and stirred overnight. The reaction mixture was treated with water and the resulting solids were collected by filtration, washed with water, and dried under reduced pressure to give 243 mg (43%) of 1-(2-{4-[6-(1-methyl-1H-pyrazol-4-yl)pyrazolo[1,5-a]pyridin-3-yl]piperazin-1-yl}pyrimidin-5-yl)propan-1-one as a gray solid. ¹H NMR (400 MHZ, CDCl₃) δ (ppm) 8.90 (s, 2H), 8.49 (s, 1H), 7.77 (s, 1H), 7.74 (s, 1H), 7.64 (s, 1H), 7.58 (d, J=9.05 Hz, 1H), 7.28 (s, 2H), 7.19 (dd, J=9.11, 1.41 Hz, 1H), 4.14-4.26 (m, 4H), 3.99 (s, 3H), 3.11-3.21 (m, 4H), 2.88 (q, J=7.34 Hz, 2H), 1.24 (t, J=7.27 Hz, 3H). MS (ESI) m/z [M+H]⁺ calcd. For C₂₂H₂₅N₈O, 417.2; found, 417.2.

### Step 3. Preparation of 1-(2-{4-[6-(1-methyl-1H-pyrazol-4-yl)pyrazolo[1,5-a]pyridin-3-yl]piperazin-1-yl}pyrimidin-5-yl)propan-1-ol (Compound 287)

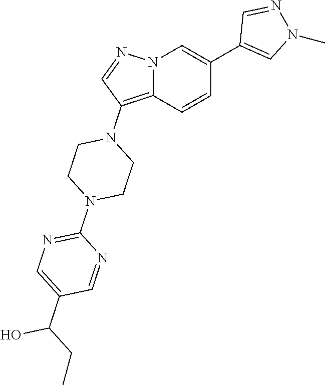 [SMILES: CCC(O)c1cnc(N2CCN(c3cnn4cc(-c5cnn(C)c5)ccc34)CC2)nc1]

To a suspension of 1-(2-{4-[6-(1-methyl-1H-pyrazol-4-yl)pyrazolo[1,5-a]pyridin-3-yl]piperazin-1-yl}pyrimidin-5-yl)propan-1-one (205 mg, 0.49 mmol, as prepared in the previous step) in MeOH (6 mL) and THF (6 mL) was added NaBH₄ (65.2 mg, 1.72 mmol) in one portion. After 30 min, the reaction mixture was partitioned between sat. aq. NaHCO₃ and EtOAc. The organic phase was washed with brine, dried (anhydrous Na₂SO₄), filtered and evaporated in vacuo. The residue was purified by silica gel chromatography eluting with a gradient of 0-50% acetone/DCM to give 53 mg (26%) of 1-(2-{4-[6-(1-methyl-1H-pyrazol-4-yl)pyrazolo[1,5-a]pyridin-3-yl]piperazin-1-yl}pyrimidin-5-yl)propan-1-ol (Compound 287) as an off white solid. ¹H NMR (400 MHZ, CDCl₃) δ (ppm) 8.48 (s, 1H), 8.36 (s, 2H), 7.69-7.81 (m, 2H), 7.54-7.67 (m, 2H), 7.18 (d, J=9.17 Hz, 1H), 4.53 (t, J=6.66 Hz, 1H), 4.02-4.14 (m, 4H), 3.99 (s, 3H), 3.16 (br. S., 4H), 2.07 (br. S., 1H), 1.89 (dt, J=13.91, 7.17 Hz, 1H), 1.69-1.83 (m, 1H), 0.97 (t, J=7.40 Hz, 3H). MS (ESI) m/z [M+H]⁺ calcd. For C₂₂H₂₇N₈O, 419.2; found, 419.2; HPLC purity: 210 nm: 82.1%; 254 nm: 99.4%.

### Example 214: Synthesis of Exemplary Compound 288

### 4-[6-(1-methyl-1H-pyrazol-4-yl)pyrazolo[1,5-a]pyridin-3-yl]-N-{(1R)-1-[4-(trifluoromethyl)phenyl]ethyl}piperazine-1-carboxamide (Compound 288)

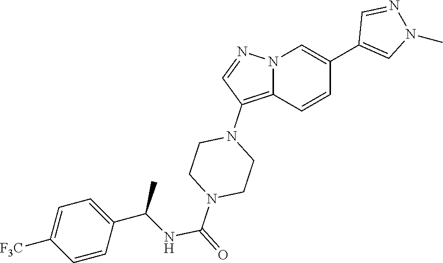 [SMILES: C[C@@H](NC(=O)N1CCN(c2cnn3cc(-c4cnn(C)c4)ccc23)CC1)c1ccc(C(F)(F)F)cc1]

### Step 1. Preparation of 1-[(1R)-1-isocyanatoethyl]-4-(trifluoromethyl) benzene

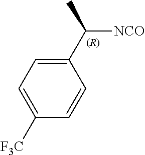

A rapidly stirred mixture of (1R)-1-[4-(trifluoromethyl)phenyl]ethanamine (500 mg, 2.64 mmol) in DCM (13 mL) and sat. aq. NaHCO₃ (13 mL) was cooled to 0° C. To this mixture was added triphosgene (0.26 g, 0.87 mmol). After 30 min at 0° C., the phases were separated. The aqueous phase was washed DCM (2×10 mL). The combined DCM layers were dried with Na₂SO₄ and concentrated under reduced pressure. The crude solid was suspended in 1:1 Et₂O/hexanes (5 mL) and filtered. The filter pad was washed with 1:1 Et₂O/hexanes (5 mL) and the filtrate concentrated in vacuo to give crude 1-[(1R)-1-isocyanatoethyl]-4-(trifluoromethyl) benzene. ¹H NMR (400 MHZ, CDCl₃) δ (ppm) 7.64 (d, J=8.19 Hz, 2H), 7.46 (d, J=8.31 Hz, 2H), 4.87 (q, J=6.72 Hz, 1H), 1.63 (d, J=6.72 Hz, 3H)

### Step 2. 4-[6-(1-methyl-1H-pyrazol-4-yl)pyrazolo[1,5-a]pyridin-3-yl]-N-{(1R)-1-[4-(trifluoromethyl)phenyl]ethyl}piperazine-1-carboxamide (Compound 288)

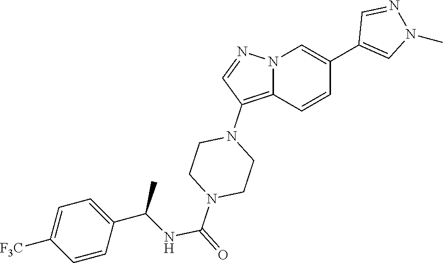 [SMILES: C[C@@H](NC(=O)N1CCN(c2cnn3cc(-c4cnn(C)c4)ccc23)CC1)c1ccc(C(F)(F)F)cc1]

To a solution of 1-[(1R)-1-isocyanatoethyl]-4-(trifluoromethyl)benzene (0.10 g, 0.47 mmol, as prepared in the previous step) in ACN (6.0 mL) was added DIPEA (0.41 mL, 2.4 mmol) followed by 6-(1-methyl-1H-pyrazol-4-yl)-3-piperazin-1-ylpyrazolo[1,5-a]pyridine hydrochloride (Compound S19) (0.19 g, 0.47 mmol). The reaction was stirred for 1.75 h at rt, then concentrated in vacuo. The residue was dissolved in DCM and washed with water (2×). The organic phase was dried with Na₂SO₄, filtered and the filtrate concentrated in vacuo. The crude product was purified by ISCO chromatography eluting with a gradient of 0%-10% MeOH in DCM. The material was stirred with 25% Et₂O in hexanes and concentrated under reduced pressure. This was repeated several times to get a yellow solid which was dried under vacuum at 50° C. overnight to give 190 mg (81%) of 4-[6-(1-methyl-1H-pyrazol-4-yl)pyrazolo[1,5-a]pyridin-3-yl]-N-{(1R)-1-[4-(trifluoromethyl)phenyl]ethyl}piperazine-1-carboxamide (Compound 288) as a yellow solid. ¹H NMR (400 MHZ, CDCl₃) δ (ppm) 8.47 (s, 1H), 7.69-7.80 (m, 2H), 7.53-7.66 (m, 4H), 7.47 (d, J=8.31 Hz, 2H), 7.18 (d, J=9.05 Hz, 1H), 5.08 (quin, J=6.85 Hz, 1H), 4.76 (d, J=6.48 Hz, 1H), 3.98 (s, 3H), 3.54-3.73 (m, 4H), 3.01-3.22 (m, 4H), 1.53 (d, J=6.97 Hz, 3H). MS (ESI) m/z [M+H]⁺ calcd. for C₂₅H₂₇F₃N₇O: 498.2, Found: 498.3; HPLC purity: 210 nm: 100.0%; 254 nm: 100.0%.

### Example 215: Synthesis of Exemplary Compound 289

### (R)-1-(pyridin-4-yl)ethyl 4-(6-(1-methyl-1H-pyrazol-4-yl)pyrazolo[1,5-a]pyridin-3-yl)piperazine-1-carboxylate (Compound 289)

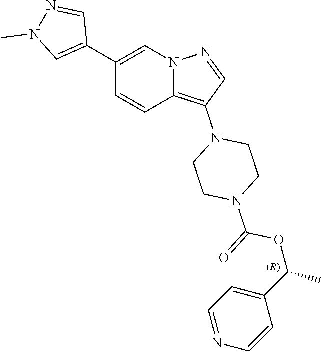

To a suspension of 4-nitrophenyl 4-[6-(1-methyl-1H-pyrazol-4-yl)pyrazolo[1,5-a]pyridin-3-yl]piperazine-1-carboxylate (0.500 g, 0.983 mmol, as prepared in Example 198, Step 1) in DMF (10 mL) was added (1R)-1-pyridin-4-ylethanol (0.1453 g, 1.180 mmol) then the mixture was cooled to 0° C., and NaH (43.26 mg, 1.082 mmol) was added. The reaction was warmed to rt and stirred for 30 min then 60% NaH mineral oil dispersion (3.933 mg, 0.09834 mmol) was added. The mixture was stirred at rt overnight then H₂O (20 mL) was added. The pH was adjusted (pH=5) by dropwise addition of AcOH then made basic with the slow addition of sat. aq. NaHCO₃. The mixture was extracted with EtOAc (3×20 mL), then NaCl was added to the aqueous layer, which was extracted with EtOAc (3×20 mL). The combined EtOAc layers were washed with 10% aqueous LiCl (×5), dried over anhydrous Na₂SO₄, and filtered. The filtrate was concentrated under reduced pressure then the residue was purified by column chromatography eluting with 0-30% IPA/DCM to afford 326.8 mg (77%) of (R)-1-(pyridin-4-yl)ethyl 4-(6-(1-methyl-1H-pyrazol-4-yl)pyrazolo[1,5-a]pyridin-3-yl)piperazine-1-carboxylate (Compound 289) as a brown solid. ¹H NMR (400 MHZ, DMSO-d₆) δ (ppm) 8.82 (s, 1H), 8.56 (d, J=5.6 Hz, 2H), 8.22 (s, 1H), 7.97 (s, 1H), 7.78 (s, 1H), 7.65 (d, J=9.2 Hz, 1H), 7.37 (d, J=5.6 Hz, 2H), 7.33 (d, J=9.2 Hz, 1H), 5.74 (q, J=6.6 Hz, 1H), 3.87 (s, 3H), 3.44-3.80 (m, 4H), 2.87-3.09 (m, 4H), 1.49 (d, J=6.6 Hz, 3H). MS (ESI) m/z [M+H]⁺ calcd. for C₂₃H₂₆N₇O₂: 432.2, Found: 432.3; HPLC purity: 210 nm: 100.0%; 254 nm: 100.0%.

### Example 216: Synthesis of Exemplary Compound 290

### (R)-1-(4-chlorophenyl)ethyl 4-(6-(1-methyl-1H-pyrazol-4-yl)pyrazolo[1,5-a]pyridin-3-yl)piperazine-1-carboxylate-2,2,3,3,5,5,6,6-d₈ (Compound 290)

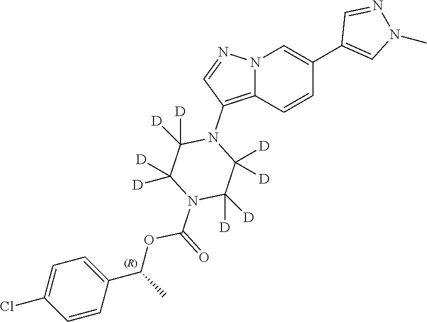

### Step 1. Preparation of tert-butyl 4-(6-(1-methyl-1H-pyrazol-4-yl)pyrazolo[1,5-a]pyridin-3-yl)piperazine-1-carboxylate-2,2,3,3,5,5,6,6-d₈

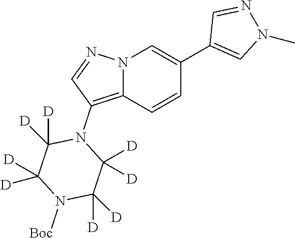 [SMILES: [2H]C1([2H])N(C(=O)OC(C)(C)C)C([2H])([2H])C([2H])([2H])N(c2cnn3cc(-c4cnn(C)c4)ccc23)C1([2H])[2H]]

A flask containing 3-bromo-6-(1-methyl-1H-pyrazol-4-yl)pyrazolo[1,5-a]pyridine (Compound S9) (951 mg, 3.43 mmol) and tert-butyl piperazine-1-carboxylate-d₈ (800 mg, 4.12 mmol) was treated with tBuOD (9.66 mL) and dioxane (4.8 mL). The solution was sparged with argon for 15 min then treated with tBuXPhos Pd G1 (353 mg, 0.515 mmol) and KOtBu (578 mg, 5.15 mmol) and the mixture was sparged for 10 min with argon. The slurry was stirred at 55° C. under argon for 22 h. The reaction mixture was cooled to rt, diluted with EtOAc (80 mL), and washed with water (2×40 mL). The organic layer was dried over anhydrous MgSO₄, filtered, and the filtrate was concentrated to a brown viscous oil. The crude material was purified by flash chromatography eluting with 15-30% acetone/DCM to afford 431 mg (32%) of tert-butyl 4-(6-(1-methyl-1H-pyrazol-4-yl)pyrazolo[1,5-a]pyridin-3-yl)piperazine-1-carboxylate-2,2,3,3,5,5,6,6-d₈ as a brownish yellow solid. ¹H NMR (400 MHZ, CDCl₃) δ (ppm) (s, 1H), 7.74 (s, 1H), 7.69 (s, 1H), 7.61 (s, 1H), 7.50 (d, J=9.17 Hz, 1H), 7.13 (d, J=9.17 Hz, 1H), 3.97 (s, 3H), 1.50 (s, 9H). MS (ESI) m/z [M+H]⁺ calcd. for C₂₀H₁₈D₈N₆O₂: 391.3, Found: 391.3.

Step 2. Preparation of 6-(1-methyl-1H-pyrazol-4-yl)-3-(piperazin-1-yl-2,2,3,3,5,5,6,6-d₈)pyrazolo[1,5-a]pyridine hydrochloride salt

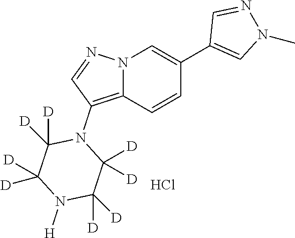 [SMILES: [2H]C1([2H])NC([2H])([2H])C([2H])([2H])N(c2cnn3cc(-c4cnn(C)c4)ccc23)C1([2H])[2H] ; Cl]

A mixture of EtOAc (7.17 mL) and absolute EtOH (0.786 mL) was cooled in an ice bath at 0° C. The solution was treated dropwise with AcCl (0.870 mL, 12.2 mmol) over 5 min then stirred for 20 min at 0° C., warmed to rt and stirred for 2 h. A solution of tert-butyl 4-(6-(1-methyl-1H-pyrazol-4-yl)pyrazolo[1,5-a]pyridin-3-yl)piperazine-1-carboxylate-2,2,3,3,5,5,6,6-d₈ (478 mg, 1.22 mmol, as prepared in the previous step) in EtOAc (5.5 mL) was added dropwise to the reaction flask. The mixture was stirred at rt for 3 h then cooled to 5° C. overnight. The reaction mixture was concentrated under reduced pressure then the residue was treated with EtOAc (15 mL) and concentrated under reduced pressure again. The resultant solid was placed under high vacuum to afford 6-(1-methyl-1H-pyrazol-4-yl)-3-(piperazin-1-yl-2,2,3,3,5,5,6,6-d₈) pyrazolo[1,5-a]pyridine hydrochloride salt as a light tan solid. MS (ESI) m/z [M+H]⁺ calcd. for C₁₅H₁₀D₈N₆: 291.2, Found: 291.3.

### Step 3. Preparation of (R)-1-(4-chlorophenyl)ethyl 4-(6-(1-methyl-1H-pyrazol-4-yl)pyrazolo[1,5-a]pyridin-3-yl)piperazine-1-carboxylate-2,2,3,3,5,5,6,6-d₈ (Compound 290)

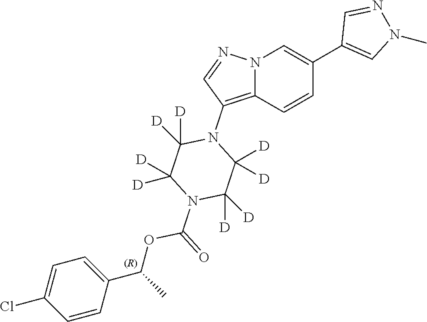

A slurry of 6-(1-methyl-1H-pyrazol-4-yl)-3-(piperazin-1-yl-2,2,3,3,5,5,6,6-da) pyrazolo[1,5-a]pyridine hydrochloride (400 mg, 1.22 mmol, as prepared in the previous step) in DCM (10 mL) was treated with a solution of (R)-1-(4-chlorophenyl)ethyl (2,5-dioxopyrrolidin-1-yl)carbonate (Compound S95) (405 mg, 1.36 mmol) and Et₃N (0.512 mL, 3.67 mmol) in DCM (2.7 mL) over 15 min. The reaction was stirred at rt for 1.5 h then diluted with DCM (20 mL) and washed with H₂O (25 mL), 0.1N HCl (25 mL), and sat. aq. NaHCO₃ (25 mL). The organic layer was dried over anhydrous Na₂SO₄, filtered, and the filtrate was concentrated under reduced pressure. The residue was purified by flash chromatography eluting with 15-30% acetone/DCM to yield a yellow foam. The yellow foam was treated with MTBE (9 mL) and hexanes (1 mL) and the mixture was allowed to stand at rt for 5 h. The resulting solid was isolated by filtration, the filter cake was washed with hexanes, and the solid was placed under high vacuum to afford 340 mg (59%) of (R)-1-(4-chlorophenyl)ethyl 4-(6-(1-methyl-1H-pyrazol-4-yl)pyrazolo[1,5-a]pyridin-3-yl)piperazine-1-carboxylate-2,2,3,3,5,5,6,6-d₈ (Compound 290) as a light yellow solid. ¹H NMR (400 MHZ, DMSO-d₆) δ (ppm) 8.81 (s, 1H), 8.21 (s, 1H), 7.96 (s, 1H), 7.76 (s, 1H), 7.64 (d, J=9.29 Hz, 1H), 7.36-7.51 (m, 4H), 7.32 (d, J=9.29 Hz, 1H), 5.73 (q, J=6.48 Hz, 1H), 3.86 (s, 3H), 1.47 (d, J=6.60 Hz, 3H). MS (ESI) m/z [M+H]⁺ calcd. for C₂₄H₁₇D₈ClN₆O₂: 473.2, Found: 473.3; HPLC purity: 210 nm: 100.0%; 254 nm: 100.0%.

### Example 217: Synthesis of Exemplary Compound 291

### 1-(4-chlorophenyl-2,3,5,6-d₄) ethyl-2,2,2-d₃ 4-(6-(1-methyl-1H-pyrazol-4-yl)pyrazolo[1,5-a]pyridin-3-yl)piperazine-1-carboxylate (Compound 291)

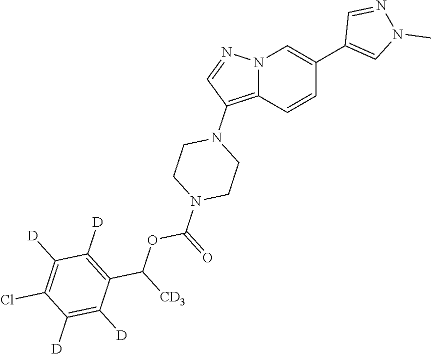 [SMILES: [2H]c1c([2H])c(C(OC(=O)N2CCN(c3cnn4cc(-c5cnn(C)c5)ccc34)CC2)C([2H])([2H])[2H])c([2H])c([2H])c1Cl]

### Step 1. Preparation of 1-(4-chlorophenyl-2,3,5,6-d4)ethan-2,2,2-d₃-1-ol

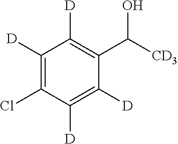 [SMILES: [2H]c1c([2H])c(C(O)C([2H])([2H])[2H])c([2H])c([2H])c1Cl]

To a solution of 1-(4-chlorophenyl)ethanone-d₇ (430 mg, 2.66 mmol) in MeOD (8.6 mL, 210 mmol) at 0° C. was added NaBH₄ (121 mg, 3.19 mmol) in one portion. The reaction was stirred at 0° C. for 20 min then quenched with 10 mL sat. aq. NaHCO₃ and allowed to warm to rt. The reaction mixture was diluted with H₂O (5 mL) and extracted with MTBE (3×15 mL). The combined organic layers were dried over anhydrous MgSO₄, filtered, and the filtrate was concentrated under reduced pressure to afford 417 mg (96%) of 1-(4-chlorophenyl-2,3,5,6-d4) ethan-2,2,2-d₃-1-ol as a colorless liquid. ¹H NMR (400 MHZ, CDCl₃) δ (ppm) 4.89 (s, 1H), 1.82 (br. s., 1H).

### Step 2. Preparation of 1-(4-chlorophenyl-2,3,5,6-d₄) ethyl-2,2,2-d₃ (2,5-dioxopyrrolidin-1-yl)carbonate

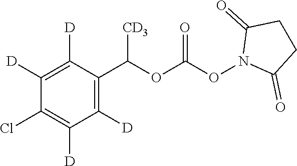 [SMILES: [2H]c1c([2H])c(C(OC(=O)ON2C(=O)CCC2=O)C([2H])([2H])[2H])c([2H])c([2H])c1Cl]

A solution of 1-(4-chlorophenyl)ethanol-d₇ (410 mg, 2.50 mmol, as prepared in the previous step) in ACN (4.50 mL) was treated with di(N-succinimidyl)carbonate (963 mg, 3.76 mmol) followed by Et₃N (1.05 mL, 7.52 mmol) and the reaction mixture was stirred at rt for 3.5 h. The reaction was concentrated under reduced pressure then the residue was dissolved in DCM and washed with sat. aq. NaHCO₃ (20 mL) and brine (20 mL), then dried over anhydrous Na₂SO₄. The solution was filtered and the filtrate was concentrated under reduced pressure to afford 792 mg (104%) of 1-(4-chlorophenyl-2,3,5,6-d₄) ethyl-2,2,2-d₃ (2,5-dioxopyrrolidin-1-yl)carbonate as a light tan, sticky solid. ¹H NMR (400 MHZ, CDCl₃) δ (ppm) 5.78 (s, 1H), 2.82 (s, 4H). HPLC purity: 210 nm: 84.5%; 254 nm: 100.0%.

### Step 3. Preparation of 1-(4-chlorophenyl-2,3,5,6-d₄) ethyl-2,2,2-d₃ 4-(6-(1-methyl-1H-pyrazol-4-yl)pyrazolo[1,5-a]pyridin-3-yl)piperazine-1-carboxylate (Compound 291)

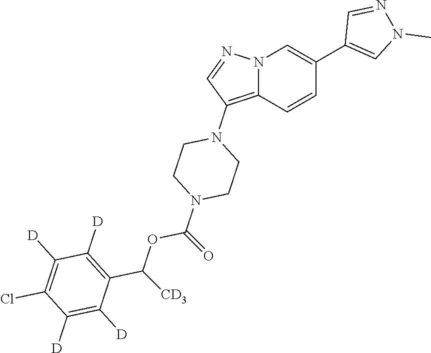 [SMILES: [2H]c1c([2H])c(C(OC(=O)N2CCN(c3cnn4cc(-c5cnn(C)c5)ccc34)CC2)C([2H])([2H])[2H])c([2H])c([2H])c1Cl]

A slurry of 6-(1-methyl-1H-pyrazol-4-yl)-3-piperazin-1-ylpyrazolo[1,5-a]pyridine hydrochloride (Compound S19) (718 mg, 2.25 mmol) in DCM (6.0 mL) was treated with a solution of 1-(4-chlorophenyl-2,3,5,6-d₄) ethyl-2,2,2-d₃ (2,5-dioxopyrrolidin-1-yl)carbonate (763 mg, 2.50 mmol, as prepared in the previous step) and Et₃N (0.471 mL, 3.38 mmol) in DCM (5.0 mL) over 15 min. The reaction mixture was stirred at rt for 1 h then Et₃N (0.5 eq) was added and for the reaction was stirred for 3 h. The mixture was diluted with DCM (20 mL) and washed with H₂O (25 mL), 0.1 N HCl (25 mL), and sat. aq. NaHCO₃ (25 mL). The organic layer was dried over anhydrous Na₂SO₄ and filtered, then the filtrate was concentrated under reduced pressure to yield the crude product. The crude product was purified by chromatography eluting with 15-25% acetone/DCM to afford a yellow foam. The foam was treated with MTBE (21 mL). The resulting slurry was heated to 50° C., and then allowed to cool to rt and stored at 2-8° C. overnight. The slurry was filtered cold and washed with hexanes to afford 605 mg (57%) of 1-(4-chlorophenyl-2,3,5,6-d₄) ethyl-2,2,2-d₃ 4-(6-(1-methyl-1H-pyrazol-4-yl)pyrazolo[1,5-a]pyridin-3-yl)piperazine-1-carboxylate (Compound 291) as a slightly yellow green solid. ¹H NMR (400 MHZ, CDCl₃) δ (ppm) 8.46 (br s, 1H), 7.74 (s, 1H), 7.70 (br s, 1H), 7.61 (s, 1H), 7.51 (d, J=9.2 Hz, 1H), 7.15 (dd, J=9.2, 1.6 Hz, 1H), 5.82 (s, 1H), 3.98 (s, 3H), 3.71 (br s, 4H), 3.04 (br s, 4H). MS (ESI) m/z [M+H]⁺ calcd. for C₂₄H₁₈D₇ClN₇O₂: 472.2, Found: 472.2; HPLC purity: 210 nm: 100.0%; 254 nm: 100.0%.

### Example 218: Synthesis of Exemplary Compound 292

### N-(4-chlorobenzyl)-4-(6-(1-methyl-1H-pyrazol-4-yl)pyrazolo[1,5-a]pyridin-3-yl)piperazine-1-sulfonamide (Compound 292)

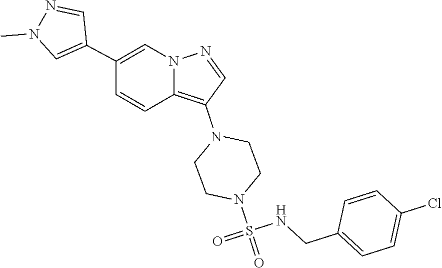 [SMILES: Cn1cc(-c2ccc3c(N4CCN(S(=O)(=O)NCc5ccc(Cl)cc5)CC4)cnn3c2)cn1]

To a mixture of 4-(chlorobenzyl)sulfamoyl fluoride (Compound S85) (93 mg, 0.42 mmol) and 6-(1-methyl-1H-pyrazol-4-yl)-3-piperazin-1-ylpyrazolo[1,5-a]pyridine (Compound 12) (127 mg, 0.45 mmol) in ACN (3.9 mL) under nitrogen was added DBU (67 μL, 0.45 mmol). The reaction was stirred at 50° C. for 2 h. The solution was cooled to rt and concentrated under reduced pressure. The residue was purified by chromatography eluting with a gradient of 0-60% DCM in acetone with 0.1% Et₃N to afford 148 mg (73%) of N-(4-chlorobenzyl)-4-(6-(1-methyl-1H-pyrazol-4-yl)pyrazolo[1,5-a]pyridin-3-yl)piperazine-1-sulfonamide (Compound 292) as a light yellow solid. ¹H NMR (400 MHZ, DMSO-d₆) δ (ppm) 8.82 (s, 1H), 8.22 (s, 1H), 7.97 (s, 1H), 7.93 (t, J=6.2 Hz, 1H), 7.75 (s, 1H), 7.63 (d, J=9.2 Hz, 1H), 7.44-7.38 (m, 4H), 7.32 (dd, J=9.4, 1.4 Hz, 1H), 4.15 (d, J=6.4 Hz, 2H), 3.87 (s, 3H), 3.22-3.19 (m, 4H), 2.99-2.97 (m, 4H). MS (ESI) m/z [M+H]⁺ calcd. for C₂₂H₂₄ClN₇O₂S: 486.2, Found: 486.2; HPLC purity: 210 nm: 100.0%; 254 nm: 100.0%.

Using the procedures described in Example 218, Compound 292 and reagents, starting materials, and conditions known to those skilled in the art were used to prepare the compounds shown in Table LL.

TABLE LL

| Compound | Name | Characterization |
|---|---|---|
| Compound | (R)-N-(1-(4-chlorophenyl)ethyl)-4-(6-(1- | ¹H NMR (400 MHz, DMSO- |
| 293 | methyl-1H-pyrazol-4-yl)pyrazolo[1,5- | d₆) δ (ppm) 8.81 (s, 1H), 8.22 |
| | a]pyridin-3-yl)piperazine-1-sulfonamide | (s, 1H) 8.00 (d, J = 8.0 Hz, |
| | | 1H), 7.96 (d, J = 0.4 Hz, 1H), |
| | | 7.70 (s, 1H), 7.60 (d, J = 8.4 |
| | | Hz, 1H), 7.42 (s, 4H), 7.31 |
| | | (dd, J = 9.2, 1.6 Hz, 1H), 4.42 |
| | | (p, J = 6.8 Hz, 1H), 3.86 (s, |
| | | 3H), 3.15-3.05 (m, 4H), 2.93- |
| | | 2.81 (m, 4H), 1.40 (d, J = 6.8 |
| | | Hz, 3H). MS (ESI) m/z |
| | | [M + H]⁺ calcd. for |
| | | C₂₃H₂₆ClN₇O₂S: 500.2, |
| | | Found: 500.2; HPLC purity: |
| | | 210 nm: 100.0%; 254 nm: |
| | | 100.0%. |
| Compound | 4-(6-(1-methyl-1H-pyrazol-4- | ¹H NMR (400 MHz, DMSO- |
| 294 | yl)pyrazolo[1,5-a]pyridin-3-yl)-N-(4- | d₆) δ (ppm) 8.82 (s, 1H), 8.22 |
| | (trifluoromethyl)benzyl)piperazine-1- | (s, 1H), 8.03 (t, J = 6.2 Hz, |
| | sulfonamide | 1H), 7.97 (d, J = 0.5 Hz, 1H), |
| | | 7.75 (s, 1H), 7.73 (s, 2H), 7.61 |
| | | (m, 3H), 7.41 (m, 4H), 7.32 |
| | | (dd, J = 9.20, 1.4 Hz, 1H), 4.26 |
| | | (d, J = 6.2 Hz, 2H), 3.87 (s, |
| | | 3H), 3.22 (m, 4H), 2.97 (m, |
| | | 4H). MS (ESI) m/z [M + H]⁺ |
| | | calcd. for C₂₃H₂₄F₃N₇O₂S: |
| | | 520.2, Found: 520.3; HPLC |
| | | purity: 210 nm: 100.0%; 254 |
| | | nm: 100.0%. |

### Example 219: Synthesis of Exemplary Compound 295

### 4-(7-(1-methyl-1H-pyrazol-4-yl)imidazo[1,2-b]pyridazin-3-yl)-N-(4-(trifluoromethyl) benzyl)piperazine-1-carboxamide (Compound 295)

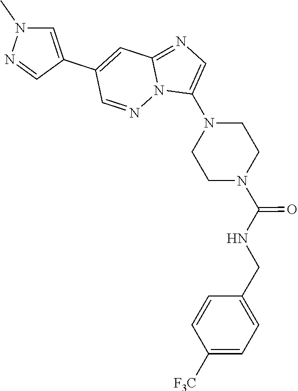 [SMILES: Cn1cc(-c2cnn3c(N4CCN(C(=O)NCc5ccc(C(F)(F)F)cc5)CC4)cnc3c2)cn1]

To a solution of 1-(isocyanatomethyl)-4-(trifluoromethyl)benzene (41.3 mg, 0.205 mmol), ACN (2.60 mL), and DIPEA (0.179 mL, 1.02 mmol) was added 7-(1-methyl-1H-pyrazol-4-yl)-3-piperazin-1-ylimidazo[1,2-b]pyridazine hydrochloride (Compound S83) (65.6 mg, 0.205 mmol). The reaction was stirred at rt overnight then concentrated under reduced pressure. The residue was dissolved in DCM, washed with water (2×), dried over anhydrous Na₂SO₄, and filtered. The filtrate was concentrated under reduced pressure and the residue was purified by flash chromatography eluting with 0-10% MeOH/DCM. The purified product was concentrated under reduced pressure then the residue was treated with 25% Et₂O/hexanes and concentrated under reduced pressure several times. The resulting solid was suspended in 25% Et₂O/hexane and filtered to afford 56.9 mg (57%) of (R)-1-(4-chlorophenyl)ethyl 4-(7-(1-methyl-1H-pyrazol-4-yl)imidazo[1,2-b]pyridazin-3-yl)piperazine-1-carboxylate (Compound 295) as a yellow solid. ¹H NMR (400 MHZ, DMSO-d₆) δ (ppm) 8.85 (d, J=2.0 Hz, 1H), 8.37 (s, 1H), 8.18 (d, J=2.4 Hz, 1H), 8.11 (s, 1H) 7.68 (d, J=8.0 Hz, 2H), 7.49 (d, J=8.0 Hz, 2H), 733 (t, J=5.6 Hz, 1H), 7.32 (s, 1H), 4.35 (d, J=5.6 Hz, 2H), 3.90 (s, 3H), 3.62-3.54 (m, 4H), 3.22-3.13 (m, 4H). MS (ESI) m/z [M+H]⁺ calcd. for C₂₃H₂₃F₃N₈O: 485.2, Found: 485.3; HPLC purity: 210 nm: 96.6%; 254 nm: 95.5%.

### Example 220: Synthesis of Exemplary Compound 296

### 4-(7-(1-methyl-1H-pyrazol-4-yl)imidazo[1,2-b]pyridazin-3-yl)-N-(4-(trifluoromethyl)benzyl)piperazine-1-sulfonamide (Compound 296)

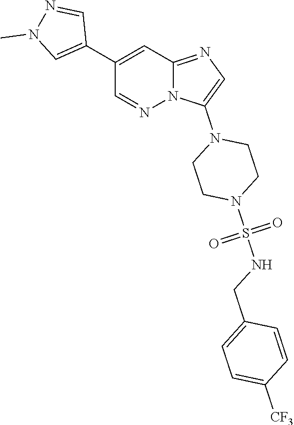 [SMILES: Cn1cc(-c2cnn3c(N4CCN(S(=O)(=O)NCc5ccc(C(F)(F)F)cc5)CC4)cnc3c2)cn1]

To a solution of 4-(trifluoromethyl)benzyl]sulfamoyl fluoride (Compound S87) (52.8 mg, 0.205 mmol) and DIPEA (0.179 mL, 0.205 mmol) in ACN (2.60 mL) was added 7-(1-methyl-1H-pyrazol-4-yl)-3-(piperazin-1-yl)imidazo[1,2-b]pyridazine hydrochloride (Compound S83) (65.6 mg, 0.205 mmol) in one portion. The reaction was heated to 50° C. overnight. The mixture was concentrated under reduced pressure and the residue was purified by flash eluting with 0-100% of 10% MeOH in DCM/DCM to afford 58 mg (54%) of 4-(7-(1-methyl-1H-pyrazol-4-yl)imidazo[1,2-b]pyridazin-3-yl)-N-(4-(trifluoromethyl) benzyl)piperazine-1-sulfonamide (Compound 296) as a yellow solid. ¹H NMR (400 MHZ, DMSO-d₆) δ (ppm) 8.82 (d, J=2.0 Hz, 1H), 8.36 (s, 1H), 8.17 (d, J=2.4 Hz, 1H), 8.10 (s, 1H), 8.05 (t, J=6.0 Hz, 1H), 7.73 (d, J=8.0 Hz, 2H), 7.60 (d, J=8.0 Hz, 2H), 7.25 (s, 1H), 4.27 (d, J=6.0 Hz, 2H), 3.89 (s, 3H), 3.28-3.24 (m, 4H), 3.22-3.16 (m, 4H). MS (ESI) m/z [M+H]⁺ calcd. for C₂₂H₂₃F₃N₈O₂S: 521.2, Found: 521.3; HPLC purity: 210 nm: 96.5%; 254 nm: 98.6%.

### Example 221: Synthesis of Exemplary Compound 297

### tert-butyl 4-(6-propionylpyrazolo[1,5-a]pyridin-3-yl)piperazine-1-carboxylate (Compound 297)

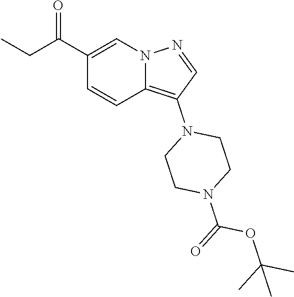 [SMILES: CCC(=O)c1ccc2c(N3CCN(C(=O)OC(C)(C)C)CC3)cnn2c1]

### Step 1. Preparation of methyl pyrazolo[1,5-a]pyridine-6-carboxylate

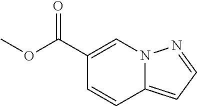

To a stirred solution of 6-bromopyrazolo[1,5-a]pyridine (3 g, 15.22 mmol) in MeOH (90 mL) were added Pd(dppf)Cl₂·DCM (1.24 g, 1.52 mmol) and Et₃N (4.62 g, 45.67 mmol) at rt. The resulting mixture was stirred overnight at 90° C. under CO (5 atm.). The resulting mixture was filtered and the filter cake was washed with DCM (3×100 mL). The filtrate was concentrated under reduced pressure. The residue was purified by silica gel column chromatography eluting with PE/EtOAc (3:1) to afford 1.88 g (70%) of methyl pyrazolo[1,5-a]pyridine-6-carboxylate as a yellow solid. ¹H NMR (400 MHZ, DMSO-d₆) δ (ppm) 9.21 (s, 1H), 8.21 (d, J=2.2 Hz, 1H), 7.79 (d, J=9.3 Hz, 1H), 7.59 (dd, J=9.3, 1.5 Hz, 1H), 6.76 (d, J=2.0 Hz, 1H), 3.89 (s, 3H). MS (ESI) m/z [M+H]⁺ calcd. for C₉H₈N₂O₂, 177.1; found, 177.2.

### Step 2. Preparation of pyrazolo[1,5-a]pyridine-6-carboxylic acid

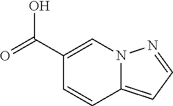 [SMILES: O=C(O)c1ccc2ccnn2c1]

To a stirred solution of methyl pyrazolo[1,5-a]pyridine-6-carboxylate (1.80 g, 10.22 mmol, as prepared in the previous step) in MeOH (6 mL) was added 1 M NaOH (30 mL) at rt. The reaction was stirred for 1 h at rt then acidified to pH 6 with conc. HCl. The resulting mixture was extracted with DCM (5×100 mL). The combined organic layers were dried over anhydrous Na₂SO₄. After filtration, the filtrate was concentrated under reduced pressure to afford 1.5 g (91%) of pyrazolo[1,5-a]pyridine-6-carboxylic acid as a yellow solid. ¹H NMR (400 MHz, DMSO-d₆) δ (ppm) 13.29 (s, 1H), 9.15 (s, 1H), 8.19 (d, J=2.2 Hz, 1H), 7.77 (d, J=9.2 Hz, 1H), 7.58 (dd, J=9.2, 1.5 Hz, 1H), 6.74 (d, J=1.8 Hz, 1H). MS (ESI) m/z [M+H]⁺ calcd. for C₈H₆N₂O₂, 163.0; found, 163.1.

### Step 3. Preparation of N-methoxy-N-methylpyrazolo[1,5-a]pyridine-6-carboxamide

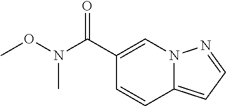

To a stirred solution of N, O-dimethylhydroxylamine hydrochloride (842.2 mg, 8.63 mmol) and pyrazolo[1,5-a]pyridine-6-carboxylic acid (1.40 g, 8.63 mmol, as prepared in the previous step) in DCM (100 mL) were added EDCl (1.99 g, 10.36 mmol), HOBt (1.40 g, 10.36 mmol), and DIPEA (4.46 g, 34.54 mmol) at rt. The reaction was stirred for 2 h at rt. The resulting mixture was filtered, the filter cake was washed with DCM (3×100 mL). The filtrate was concentrated under reduced pressure. The residue was purified by silica gel column chromatography eluting with PE/EtOAc (1:1) to afford 1.2 g (71%) of N-methoxy-N-methylpyrazolo[1,5-a]pyridine-6-carboxamide as a yellow oil. ¹H NMR (400 MHZ, DMSO-d₆) δ (ppm) 9.02 (s, 1H), 8.14 (d, J=2.2 Hz, 1H), 7.75 (d, J=10.0 Hz, 1H), 7.44 (dd, J=9.2, 1.5 Hz, 1H), 6.71 (d, J=2.8 Hz, 1H), 3.64 (s, 3H), 3.32 (s, 3H). MS (ESI) m/z [M+H]⁺ calcd. for C₁₀H₁₁N₃O₂, 206.1; found, 206.2.

### Step 4. Preparation of 1-(pyrazolo[1,5-a]pyridin-6-yl)propan-1-one

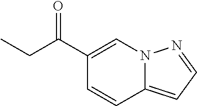

To a stirred solution of N-methoxy-N-methylpyrazolo[1,5-a]pyridine-6-carboxamide (1.20 g, 5.94 mmol, as prepared in the previous step) in THF (150 mL) was added a solution of EtMgBr (59.69 mL) at 0° C. under nitrogen. The reaction was stirred for 2 h at 0° C. under nitrogen then quenched with sat. aq. NH₄Cl at 0° C. The resulting mixture was concentrated under reduced pressure and extracted with DCM (3×100 mL). The combined organic layers were dried over anhydrous Na₂SO₄. After filtration, the filtrate was concentrated under reduced pressure. The residue was purified by silica gel column chromatography eluting with PE/EtOAc (1:1) to afford 400 mg (39%) 1-(pyrazolo[1,5-a]pyridin-6-yl)propan-1-one as a yellow solid. ¹H NMR (400 MHZ, DMSO-d₆) δ (ppm) 9.47 (s, 1H), 8.22 (d, J=2.1 Hz, 1H), 7.76 (d, J=9.3 Hz, 1H), 7.62 (dd, J=9.3, 1.6 Hz, 1H), 6.74 (d, J=1.7 Hz, 1H), 3.20-3.03 (m, 2H), 1.11 (t, J=7.2 Hz, 3H). MS (ESI) m/z [M+H]⁺ calcd. for C₁₀H₁₀N₂O, 175.1; found, 175.2.

### Step 5. Preparation of 1-(3-bromopyrazolo[1,5-a]pyridin-6-yl)propan-1-one

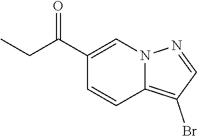

To a stirred solution of 1-(pyrazolo[1,5-a]pyridin-6-yl)propan-1-one (220 mg, 1.26 mmol, as prepared in the previous step) in DCM (5 mL) was added NBS (247.2 mg, 1.39 mmol) at rt. The reaction was stirred for 1 h at rt then purified by silica gel column chromatography eluting with PE/EtOAc (5:1) to afford 250 mg (78%) of 1-(3-bromopyrazolo[1,5-a]pyridin-6-yl)propan-1-one as a white solid. ¹H NMR (400 MHZ, DMSO-d₆) δ (ppm) 9.51 (s, 1H), 8.39 (s, 1H), 7.74 (dd, J=9.3, 1.5 Hz, 1H), 7.64 (d, J=9.3 Hz, 1H), 3.21-3.01 (m, 2H), 1.11 (t, J=7.2 Hz, 3H). MS (ESI) m/z [M+H]⁺ calcd. for C₁₀H₉BrN₂O, 253.0; found, 253.1.

### Step 6. Preparation of tert-butyl 4-(6-propionylpyrazolo[1,5-a]pyridin-3-yl)piperazine-1-carboxylate (Compound 297)

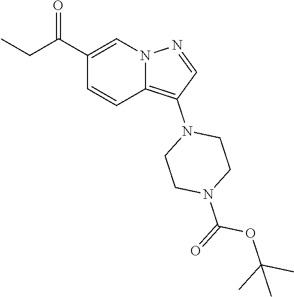 [SMILES: CCC(=O)c1ccc2c(N3CCN(C(=O)OC(C)(C)C)CC3)cnn2c1]

To a stirred solution of 1-(3-bromopyrazolo[1,5-a]pyridin-6-yl)propan-1-one (230 mg, 0.91 mmol, as prepared in the previous step) and tert-butyl piperazine-1-carboxylate (846.3 mg, 4.54 mmol) in tBuOH (6 mL) and dioxane (3 mL) were added KOtBu (152.9 mg, 1.36 mmol) and tBuXPhos Pd G1 (124.8 mg, 0.18 mmol) at rt. The reaction was stirred for 16 h at 50° C. under nitrogen. The resulting mixture was concentrated under reduced pressure. The residue was dissolved in water (300 mL). The resulting mixture was extracted with EtOAc (3×80 mL). The combined organic layers were dried over anhydrous Na₂SO₄. After filtration, the filtrate was concentrated under reduced pressure. The residue was purified by Prep-TLC eluting with DCM/MeOH (10:1) to afford crude product. The crude product was purified by Prep-HPLC to afford 5.5 mg (2%) of tert-butyl 4-(6-propionylpyrazolo[1,5-a]pyridin-3-yl)piperazine-1-carboxylate (Compound 297) as a yellow solid. ¹H NMR (400 MHZ, DMSO-d₆) δ (ppm) 9.31 (s, 1H), 8.07 (s, 1H), 7.73-7.66 (m, 1H), 7.44 (dd, J=9.4, 1.5 Hz, 1H), 3.50 (t, J=4.9 Hz, 4H), 3.07 (q, J=7.2 Hz, 2H), 2.97 (t, J=5.1 Hz, 4H), 1.42 (s, 9H), 1.09 (t, J=7.2 Hz, 3H). MS (ESI) m/z [M+H]⁺ calcd. for C₁₉H₂₆N₄O₃, 359.2; found, 359.2. HPLC purity: 254 nm: 98.6%.

### Example 222: Synthesis of Exemplary Compound 298

### tert-butyl 4-(6-(1-hydroxypropyl)pyrazolo[1,5-a]pyridin-3-yl)piperazine-1-carboxylate (Compound 298)

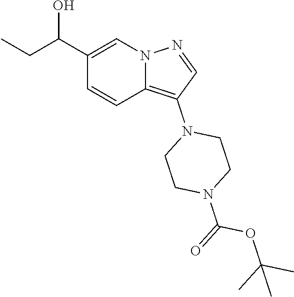 [SMILES: CCC(O)c1ccc2c(N3CCN(C(=O)OC(C)(C)C)CC3)cnn2c1]

To a stirred solution of tert-butyl 4-(6-propionylpyrazolo[1,5-a]pyridin-3-yl)piperazine-1-carboxylate (Compound 297) (50 mg, 0.14 mmol) in MeOH (4 mL) was added NaBH₄ (5.8 mg, 0.15 mmol) at 0° C. The reaction was stirred for 30 min at rt then quenched by the addition of sat. aq. NH₄Cl (1 mL) at 0° C. The residue was purified by reversed-phase flash chromatography to afford 12.5 mg (25%) of tert-butyl 4-(6-(1-hydroxypropyl)pyrazolo[1,5-a]pyridin-3-yl)piperazine-1-carboxylate (Compound 298) as a yellow solid. ¹H NMR (400 MHz, DMSO-d₆) δ 8.36 (s, 1H), 7.76 (s, 1H), 7.59 (d, J=9.2 Hz, 1H), 7.07 (d, J=9.2 Hz, 1H), 5.28 (d, J=4.5 Hz, 1H), 4.56-4.42 (m, 1H), 3.50 (t, J=4.9 Hz, 4H), 2.92 (t, J=5.0 Hz, 4H), 1.74-1.60 (m, 2H), 1.43 (s, 9H), 0.84 (t, J=7.4 Hz, 3H). MS (ESI) m/z [M+H]⁺ calcd. for C₁₉H₂₈N₄O₃, 361.2; found, 361.20; HPLC purity: 254 nm: 98.0%.

### Example 223: Synthesis of Exemplary Compound 299

### 3-(5-benzylpyrimidin-2-yl)-1-(6-(1-methyl-1H-pyrazol-4-yl)pyrazolo[1,5-a]pyridin-3-yl)propan-1-ol (Compound 299)

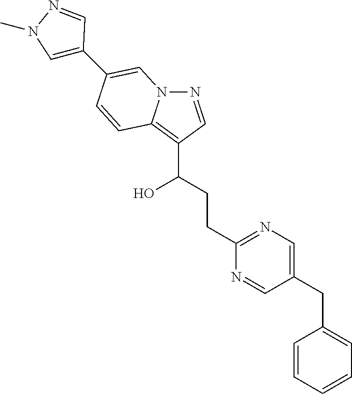 [SMILES: Cn1cc(-c2ccc3c(C(O)CCc4ncc(Cc5ccccc5)cn4)cnn3c2)cn1]

### Step 1. Preparation of 5-benzyl-2-(3-((tert-butyldimethylsilyl)oxy)prop-1-en-1-yl)pyrimidine

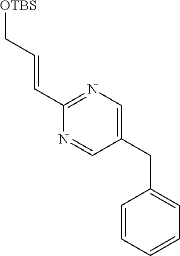 [SMILES: CC(C)(C)[Si](C)(C)OC/C=C/c1ncc(Cc2ccccc2)cn1]

To a stirred solution of 5-benzyl-2-chloropyrimidine (Compound S88) (4 g, 19.56 mmol) and tert-butyldimethyl((3-(4,4,5,5-tetramethyl-1,3,2-dioxaborolan-2-yl) allyl)oxy) silane (8.76 g, 29.32 mmol) in dioxane (60 mL) and H₂O (6 mL) were added Pd(dppf)Cl₂·DCM (1.59 g, 1.80 mmol) and Cs₂CO₃ (19.12 g, 58.64 mmol) at rt. The reaction was stirred overnight at 90° C. under nitrogen. The resulting mixture was concentrated under reduced pressure. The residue was dissolved in water (200 mL). The resulting mixture was extracted with DCM (3×100 mL). The combined organic layers were dried over anhydrous Na₂SO₄. After filtration, the filtrate was concentrated under reduced pressure. The residue was purified by silica gel column chromatography eluting with PE/EtOAc (5:1) to afford 5.8 g (87%) of 5-benzyl-2-(3-((tert-butyldimethylsilyl)oxy)prop-1-en-1-yl)pyrimidine as a yellow oil. ¹H NMR (400 MHZ, DMSO-d₆) δ (ppm) 8.65 (s, 2H), 7.36-7.25 (m, 4H), 7.25-7.18 (m, 1H), 7.13-7.02 (m, 1H), 6.69-6.59 (m, 1H), 4.45-4.34 (m, 2H), 3.96 (s, 2H), 0.91 (s, 9H), 0.08 (s, 6H). MS (ESI) m/z [M+H]⁺ calcd. for C₂₀H₂₈N₂OSi, 341.2; found, 341.3.

### Step 2. Preparation of 5-benzyl-2-(3-((tert-butyldimethylsilyl)oxy)propyl)pyrimidine

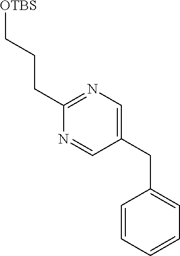 [SMILES: CC(C)(C)[Si](C)(C)OCCCc1ncc(Cc2ccccc2)cn1]

To a stirred solution of 5-benzyl-2-(3-((tert-butyldimethylsilyl)oxy)prop-1-en-1-yl)pyrimidine (5.5 g, 16.15 mmol, as prepared in the previous step) in MeOH (70 mL) was added Pd/C (343.7 mg, 3.23 mmol). The reaction was stirred for 2 h at rt under hydrogen atmosphere. The resulting mixture was filtered, the filter cake was washed with DCM (3×100 mL). The filtrate was concentrated under reduced pressure to afford 5 g (90%) of 5-benzyl-2-(3-((tert-butyldimethylsilyl)oxy)propyl)pyrimidine as a yellow oil. ¹H NMR (400 MHZ, DMSO-d₆) δ (ppm) 8.64 (s, 2H), 7.46-7.12 (m, 5H), 3.97 (s, 2H), 3.65 (t, J=6.3 Hz, 2H), 2.90 (t, J=7.6 Hz, 2H), 2.00-1.86 (m, 2H), 0.86 (s, 9H), 0.02 (s, 6H). MS (ESI) m/z [M+H]⁺ calcd. for C₂₀H₃₀N₂OSi, 343.2; found, 343.3.

### Step 3. Preparation of 3-(5-benzylpyrimidin-2-yl)propan-1-ol

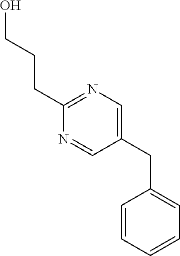 [SMILES: OCCCc1ncc(Cc2ccccc2)cn1]

To a stirred solution of 5-benzyl-2-(3-((tert-butyldimethylsilyl)oxy)propyl)pyrimidine (4.9 g, 14.30 mmol, as prepared in the previous step) in DCM (10 mL) was added 4 M HCl in dioxane (37 mL) at 0° C. The reaction was stirred for 3 h at rt. The resulting mixture was concentrated under reduced pressure. The residue was dissolved in water (100 mL). The mixture was basified to pH 8 with sat. aq. NaHCO₃. The resulting mixture was extracted with DCM (3×80 mL). The combined organic layers were dried over anhydrous Na₂SO₄. After filtration, the filtrate was concentrated under reduced pressure to afford 3.1 g (95%) of 3-(5-benzylpyrimidin-2-yl)propan-1-ol as a yellow solid. ¹H NMR (400 MHZ, DMSO-d₆) δ (ppm) 8.61 (s, 2H), 7.44-7.10 (m, 5H), 4.51-4.43 (m, 1H), 3.94 (s, 2H), 3.49-3.38 (m, 2H), 2.86 (t, J=7.0 Hz, 2H), 1.92-1.78 (m, 2H). MS (ESI) m/z [M+H]⁺ calcd. for C₁₄H₁₆N₂O, 229.1; found, 229.2.

### Step 4. Preparation of 3-(5-benzylpyrimidin-2-yl)propanal

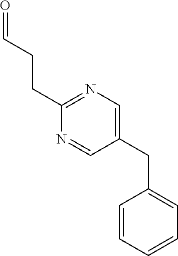 [SMILES: O=CCCc1ncc(Cc2ccccc2)cn1]

To a stirred solution of 3-(5-benzylpyrimidin-2-yl)propan-1-ol (1 g, 4.38 mmol, as prepared in the previous step) in DCM (25 mL) was added Dess-Martin periodinane (1.86 g, 4.38 mmol) at 0° C. The reaction was stirred for 1 h at rt. The resulting mixture was filtered, and the filter cake was washed with DCM (3×70 mL). The filtrate was washed with sat. aq. NaHCO₃ (3×70 mL). The organic layer was dried over anhydrous Na₂SO₄. After filtration, the filtrate was concentrated under reduced pressure. The residue was purified by silica gel column chromatography eluting with PE/EtOAc (1:1) to afford 600 mg (60.5%) of 3-(5-benzylpyrimidin-2-yl)propanal as a yellow oil. ¹H NMR (400 MHZ, DMSO-d₆) δ (ppm) 9.79 (s, 1H), 8.65 (s, 2H), 7.43-7.15 (m, 5H), 3.98 (s, 2H), 3.18 (t, J=6.8 Hz, 2H), 2.97-2.89 (m, 2H). MS (ESI) m/z [M+H]⁺ calcd. for C₁₄H₁₄N₂O, 227.1; found, 227.2.

### Step 5. Preparation of 3-(5-benzylpyrimidin-2-yl)-1-(6-(1-methyl-1H-pyrazol-4-yl)pyrazolo[1,5-a]pyridin-3-yl)propan-1-ol (Compound 299)

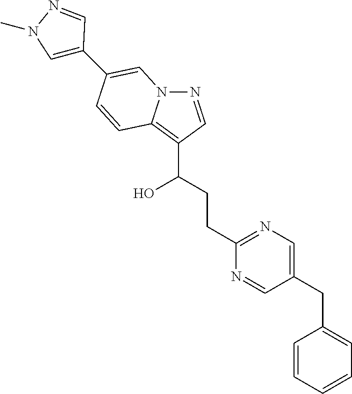 [SMILES: Cn1cc(-c2ccc3c(C(O)CCc4ncc(Cc5ccccc5)cn4)cnn3c2)cn1]

To a stirred solution of 3-iodo-6-(1-methyl-1H-pyrazol-4-yl)pyrazolo[1,5-a]pyridine (701.9 mg, 2.17 mmol, as prepared for Compound S57, Step 1) in THF (20 mL) was added i-PrMgCl·LiCl (2.5 mL, 1.3 M) at 0° C. under nitrogen. The resulting mixture was stirred for 1 h at rt under nitrogen. To the above mixture was added 3-(5-benzylpyrimidin-2-yl)propanal (140 mg, 0.62 mmol, as prepared in the previous step) at 0° C. The reaction was stirred for 0.5 h at 0° C. then quenched with sat. aq. NH₄Cl at 0° C. The resulting mixture was diluted with water (50 mL), concentrated under reduced pressure, and extracted with DCM (3×50 mL). The combined organic layers were dried over anhydrous Na₂SO₄. After filtration, the filtrate was concentrated under reduced pressure. The residue was purified by silica gel column chromatography eluting with DCM/MeOH (15:1) and then purified by Prep-HPLC to afford 47.8 mg (18%) of 3-(5-benzylpyrimidin-2-yl)-1-(6-(1-methyl-1H-pyrazol-4-yl)pyrazolo[1,5-a]pyridin-3-yl)propan-1-ol (Compound 299) as a white solid. ¹H NMR (400 MHz, DMSO-d₆) δ (ppm) 8.91 (s, 1H), 8.60 (s, 2H), 8.23 (s, 1H), 7.98 (s, 1H), 7.88 (s, 1H), 7.73 (d, J=8.9 Hz, 1H), 7.42 (dd, J=9.2, 1.6 Hz, 1H), 7.35-7.17 (m, 5H), 5.21 (d, J=4.7 Hz, 1H), 5.00-4.86 (m, 1H), 3.94 (s, 2H), 3.88 (s, 3H), 3.08-2.73 (m, 2H), 2.32-2.11 (m, 2H). MS (ESI) m/z [M+H]⁺ calcd. for C₂₅H₂₄N₆O, 425.2; found, 425.35; HPLC purity: 254 nm: 98.0%.

### Example 224: Synthesis of Exemplary Compound 300

### 3-(4-(5-benzylpyrimidin-2-yl)-3,6-dihydropyridin-1 (2H)-yl)-6-(1-methyl-1H-pyrazol-4-yl)pyrazolo[1,5-a]pyridine (Compound 300)

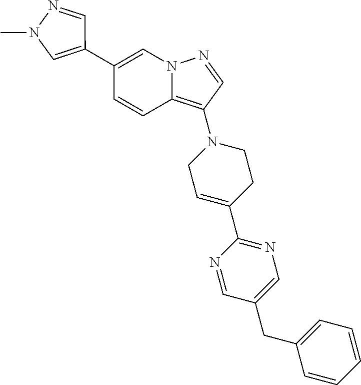 [SMILES: Cn1cc(-c2ccc3c(N4CC=C(c5ncc(Cc6ccccc6)cn5)CC4)cnn3c2)cn1]

### Step 1. Preparation of 8-(6-(1-methyl-1H-pyrazol-4-yl)pyrazolo[1,5-a]pyridin-3-yl)-1,4-dioxa-8-azaspiro[4.5]decane

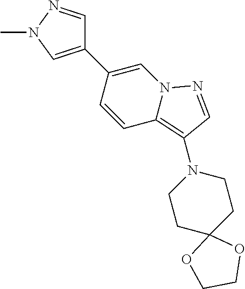 [SMILES: Cn1cc(-c2ccc3c(N4CCC5(CC4)OCCO5)cnn3c2)cn1]

To a stirred solution of 1,4-dioxa-8-azaspiro[4.5]decane (10.08 g, 70.36 mmol) and 3-bromo-6-(1-methyl-1H-pyrazol-4-yl)pyrazolo[1,5-a]pyridine (Compound S9) (3.9 g, 14.07 mmol) in tBuOH (300 mL) and dioxane (150 mL) were added KOtBu (2.37 g, 21.11 mmol) and tBuXPhos Pd G1 (1.93 g, 2.81 mmol) at rt. The reaction was stirred for 16 h at 50° C. under nitrogen then concentrated under reduced pressure. The residue was dissolved in H₂O (500 mL). The resulting mixture was extracted with DCM (3×100 mL). The combined organic layers were dried over anhydrous Na₂SO₄. After filtration, the filtrate was concentrated under reduced pressure. The residue was purified by silica gel column chromatography eluting with PE/EtOAc (1:1) to afford 2.8 g (59%) of 8-(6-(1-methyl-1H-pyrazol-4-yl)pyrazolo[1,5-a]pyridin-3-yl)-1,4-dioxa-8-azaspiro[4.5]decane as a yellow solid. ¹H NMR (400 MHZ, DMSO-d₆) δ (ppm) 8.80 (s, 1H), 8.22 (s, 1H), 7.96 (s, 1H), 7.76 (s, 1H), 7.61 (d, J=9.2 Hz, 1H), 7.30 (d, J=9.2 Hz, 1H), 3.92 (s, 4H), 3.87 (s, 3H), 3.07 (t, J=5.2 Hz, 4H), 1.81 (t, J=5.4 Hz, 4H). MS (ESI) m/z [M+H]⁺ calcd. for C₁₈H₂₁N₅O₂, 340.2; found, 340.3.

### Step 2. Preparation of 1-(6-(1-methyl-1H-pyrazol-4-yl)pyrazolo[1,5-a]pyridin-3-yl)piperidin-4-one

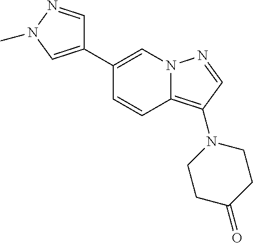 [SMILES: Cn1cc(-c2ccc3c(N4CCC(=O)CC4)cnn3c2)cn1]

To a stirred solution of 8-(6-(1-methyl-1H-pyrazol-4-yl)pyrazolo[1,5-a]pyridin-3-yl)-1,4-dioxa-8-azaspiro[4.5]decane (2.80 g, 8.25 mmol, as prepared in the previous step) in THF (3 mL) was added conc. HCl (30 mL) at 0° C. The reaction was stirred for 5 h at rt then basified to pH 8 with NaOH. The resulting mixture was extracted with DCM (3×100 mL). The combined organic layers were dried over anhydrous Na₂SO₄. After filtration, the filtrate was concentrated under reduced pressure. The residue was purified by silica gel column chromatography eluting with PE/EtOAc (1:10) to afford 890 mg (37%) of 1-(6-(1-methyl-1H-pyrazol-4-yl)pyrazolo[1,5-a]pyridin-3-yl)piperidin-4-one as a yellow solid. ¹H NMR (400 MHz, DMSO-d₆) δ (ppm) 8.84 (s, 1H), 8.24 (s, 1H), 7.98 (s, 1H), 7.83 (s, 1H), 7.74-7.69 (m, 1H), 7.35 (dd, J=9.2, 1.5 Hz, 1H), 3.87 (s, 3H), 3.35 (t, J=6.1 Hz, 4H), 2.55 (t, J=6.0 Hz, 4H). MS (ESI) m/z [M+H]⁺ calcd. for C₁₆H₁₇N₅O, 296.1; found, 296.2.

### Step 3. Preparation of 1-(6-(1-methyl-1H-pyrazol-4-yl)pyrazolo[1,5-a]pyridin-3-yl)-1,2,3,6-tetrahydropyridin-4-yl trifluoromethanesulfonate

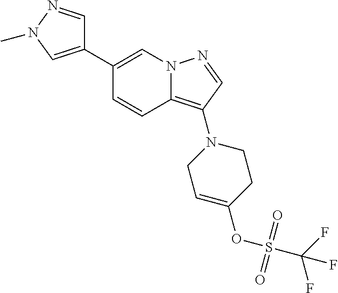 [SMILES: Cn1cc(-c2ccc3c(N4CC=C(OS(=O)(=O)C(F)(F)F)CC4)cnn3c2)cn1]

To a stirred solution of 1-(6-(1-methyl-1H-pyrazol-4-yl)pyrazolo[1,5-a]pyridin-3-yl)piperidin-4-one (600 mg, 2.03 mmol, as prepared in the previous step) in THF (70 mL) was added 1M LiHMDS (11 mL) at −78° C. under nitrogen. The reaction was stirred for 1 h at −78° C. under nitrogen then 1,1,1-trifluoro-N-phenyl-N-((trifluoromethyl)sulfonyl) methanesulfonamide (943.3 mg, 2.65 mmol) was added at −78° C. The reaction was stirred overnight at rt then quenched with sat. aq. NH₄Cl at −30° C. The resulting mixture was extracted with DCM (3×100 mL). The combined organic layers were dried over anhydrous Na₂SO₄. After filtration, the filtrate was concentrated under reduced pressure. The residue was purified by Prep-TLC (PE/EtOAc 1:10) to afford 340 mg (38%) of 1-(6-(1-methyl-1H-pyrazol-4-yl)pyrazolo[1,5-a]pyridin-3-yl)-1,2,3,6-tetrahydropyridin-4-yl trifluoromethanesulfonate as a yellow solid. ¹H NMR (400 MHZ, DMSO-d₆) δ (ppm) 8.84 (s, 1H), 8.24 (s, 1H), 7.98 (s, 1H), 7.81 (s, 1H), 7.69 (d, J=9.3 Hz, 1H), 7.35 (dd, J=9.2, 1.2 Hz, 1H), 6.19-5.98 (m, 1H), 3.88 (s, 3H), 3.79-3.61 (m, 2H), 3.30 (t, J=5.6 Hz, 2H), 2.62-2.55 (m, 2H). MS (ESI) m/z [M+H]⁺ calcd. for C₁₇H₁₆F₃N₅O₀₃S, 428.1; found, 428.2.

### Step 4. Preparation of 6-(1-methyl-1H-pyrazol-4-yl)-3-(4-(4,4,5,5-tetramethyl-1,3,2-dioxaborolan-2-yl)-3,6-dihydropyridin-1 (2H)-yl)pyrazolo[1,5-a]pyridine

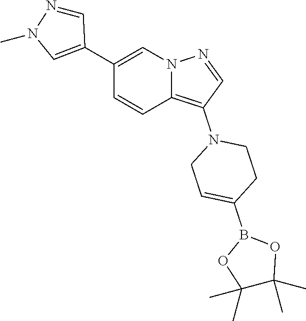

To a stirred solution of 1-(6-(1-methyl-1H-pyrazol-4-yl)pyrazolo[1,5-a]pyridin-3-yl)-1,2,3,6-tetrahydropyridin-4-yl trifluoromethanesulfonate (100 mg, 0.23 mmol, as prepared in the previous step) and 4,4,4′,4′,5,5,5′,5′-octamethyl-2,2′-bi (1,3,2-dioxaborolane) (178.2 mg, 0.70 mmol) in dioxane (6 mL) were added XPhos (11.1 mg, 0.02 mmol), XPhos Pd G3 (19.8 mg, 0.02 mmol) and KOAc (91.8 mg, 0.94 mmol) at rt. The reaction was stirred for 1 h at 90° C. under nitrogen then concentrated under reduced pressure. The residue was dissolved in water (50 mL). The resulting mixture was extracted with DCM (3×80 mL). The combined organic layers were dried over anhydrous Na₂SO₄. After filtration, the filtrate was concentrated under reduced pressure. The crude product was used in the next step without further purification. MS (ESI) m/z [M+H]⁺ calcd. for C₂₂H₂₈BN₅O₂, 406.2; found, 406.3.

### Step 5. Preparation of 3-(4-(5-benzylpyrimidin-2-yl)-3,6-dihydropyridin-1 (2H)-yl)-6-(1-methyl-1H-pyrazol-4-yl)pyrazolo[1,5-a]pyridine (Compound 300)

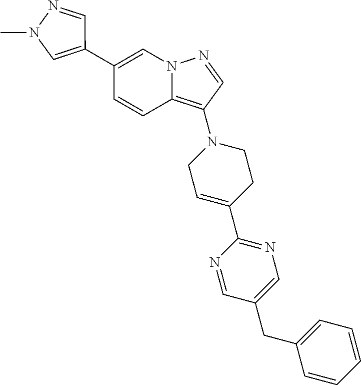 [SMILES: Cn1cc(-c2ccc3c(N4CC=C(c5ncc(Cc6ccccc6)cn5)CC4)cnn3c2)cn1]

To a stirred solution of 6-(1-methyl-1H-pyrazol-4-yl)-3-(4-(4,4,5,5-tetramethyl-1,3,2-dioxaborolan-2-yl)-3,6-dihydropyridin-1 (2H)-yl)pyrazolo[1,5-a]pyridine (95.1 mg, 0.23 mmol, as prepared in the previous step) and 5-benzyl-2-chloropyrimidine (Compound S88) (24 mg, 0.12 mmol) in dioxane (5 mL) and H₂O (1 mL) were added XPhos (5.6 mg, 0.01 mmol), XPhos Pd G3 (9.9 mg, 0.01 mmol) and K₃PO₄ (49.8 mg, 0.23 mmol) at rt. The reaction was stirred for 2 h at 100° C. under nitrogen then concentrated under reduced pressure. The residue was dissolved in water (60 mL). The resulting mixture was extracted with DCM (3×50 mL). The combined organic layers were dried over anhydrous Na₂SO₄. After filtration, the filtrate was concentrated under reduced pressure. The residue was purified by Prep-TLC (PE/EtOAc 1:10) and then purified by Prep-HPLC to afford 21.2 mg (39%) of 3-(4-(5-benzylpyrimidin-2-yl)-3,6-dihydropyridin-1 (2H)-yl)-6-(1-methyl-1H-pyrazol-4-yl)pyrazolo[1,5-a]pyridine (Compound 300) as a yellow solid. ¹H NMR (400 MHZ, DMSO-d₆) δ (ppm) 8.83 (s, 1H), 8.72 (s, 2H), 8.23 (s, 1H), 7.98 (s, 1H), 7.81 (s, 1H), 7.71 (d, J=9.2 Hz, 1H), 7.41-7.18 (m, 7H), 4.00 (s, 2H), 3.88 (s, 3H), 3.88-3.81 (m, 2H), 3.28 (t, J=5.5 Hz, 2H), 2.83-2.71 (m, 2H). MS (ESI) m/z [M+H]⁺ calcd. for C₂₇H₂₅N₇, 448.2; found, 448.25; HPLC purity: 254 nm: 95.2%.

### Example 225: Synthesis of Exemplary Compound 301

### 3-((2-(4-(6-(1-methyl-1H-pyrazol-4-yl)pyrazolo[1,5-a]pyridin-3-yl)piperazin-1-yl)pyrimidin-5-yl)methyl)-1,2,4-oxadiazole (Compound 301)

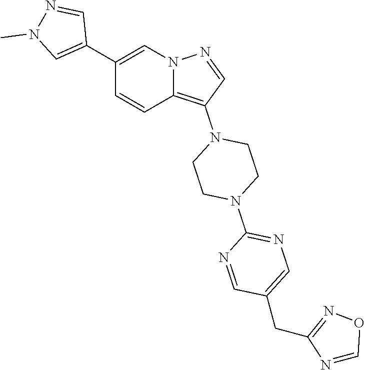 [SMILES: Cn1cc(-c2ccc3c(N4CCN(c5ncc(Cc6ncon6)cn5)CC4)cnn3c2)cn1]

### Step 1. Preparation of (2-(4-(6-(1-methyl-1H-pyrazol-4-yl)pyrazolo[1,5-a]pyridin-3-yl)piperazin-1-yl)pyrimidin-5-yl) boronic acid

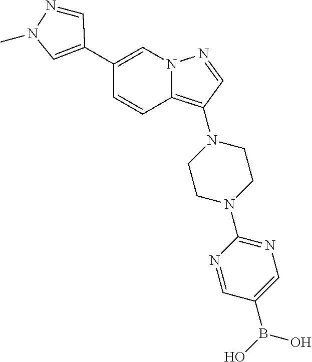 [SMILES: Cn1cc(-c2ccc3c(N4CCN(c5ncc(B(O)O)cn5)CC4)cnn3c2)cn1]

To a solution of 2-chloropyrimidin-5-ylboronic acid (560.8 mg, 3.54 mmol) and 1-[6-(1-methylpyrazol-4-yl)pyrazolo[1,5-a]pyridin-3-yl]piperazine (Compound 12) (500 mg, 1.77 mmol) in IPA (20 mL) was added DIPEA (228.9 mg, 1.77 mmol). After stirring for 6 h at 90° C. under nitrogen, the mixture was allowed to cool to rt. The resulting mixture was concentrated under reduced pressure. The product was precipitated by the addition of EtOAc. The residue was purified by trituration with H₂O (100 mL) to afford 290 mg (41%) of (2-(4-(6-(1-methyl-1H-pyrazol-4-yl)pyrazolo[1,5-a]pyridin-3-yl)piperazin-1-yl)pyrimidin-5-yl) boronic acid as a brown solid. ¹H NMR (400 MHZ, DMSO-d₆) δ (ppm) 8.84 (s, 1H), 8.66 (s, 2H), 8.24 (s, 1H), 8.09 (s, 2H), 7.98 (s, 1H), 7.79 (s, 1H), 7.70 (d, J=9.2 Hz, 1H), 7.34 (d, J=9.2 Hz, 1H), 3.98 (t, J=4.9 Hz, 4H), 3.88 (s, 3H), 3.05 (t, J=5.0 Hz, 4H). MS (ESI) m/z [M+H]⁺ calcd. for C₁₉H₂₁BN₈O₂, 405.1; found 405.1.

### Step 2. 3-((2-(4-(6-(1-methyl-1H-pyrazol-4-yl)pyrazolo[1,5-a]pyridin-3-yl)piperazin-1-yl)pyrimidin-5-yl)methyl)-1,2,4-oxadiazole (Compound 301)

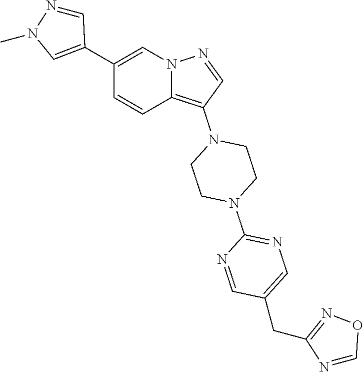 [SMILES: Cn1cc(-c2ccc3c(N4CCN(c5ncc(Cc6ncon6)cn5)CC4)cnn3c2)cn1]

To a solution of (2-(4-(6-(1-methyl-1H-pyrazol-4-yl)pyrazolo[1,5-a]pyridin-3-yl)piperazin-1-yl)pyrimidin-5-yl) boronic acid (102.3 mg, 0.25 mmol, as prepared in the previous step) and 3-(chloromethyl)-1,2,4-oxadiazole (30 mg, 0.25 mmol) in DMSO (10 mL) were added XPhos (12.1 mg, 0.03 mmol) and XPhos Pd G3 (21.4 mg, 0.03 mmol) and K₃PO₄ (107.5 mg, 0.51 mmol). The reaction was stirred overnight at 60° C. under nitrogen atmosphere. The mixture was allowed to cool to rt and concentrated under reduced pressure. The residue was dissolved in H₂O (20 mL). The resulting mixture was extracted with EtOAc (3×30 mL) and dried over anhydrous Na₂SO₄. After filtration, the filtrate was concentrated under reduced pressure. The crude product was purified by Prep-HPLC to afford 4.5 mg (4%) of 3-((2-(4-(6-(1-methyl-1H-pyrazol-4-yl)pyrazolo[1,5-a]pyridin-3-yl)piperazin-1-yl)pyrimidin-5-yl)methyl)-1,2,4-oxadiazole (Compound 301) as a white solid. ¹H NMR (400 MHZ, DMSO-d₆) δ (ppm) 9.54 (s, 1H), 8.83 (s, 1H), 8.38 (s, 2H), 8.24 (s, 1H), 7.98 (s, 1H), 7.79 (s, 1H), 7.70 (d, J=9.2 Hz, 1H), 7.34 (d, J=9.2 Hz, 1H), 4.04 (s, 2H), 3.93 (t, J=5.0 Hz, 4H), 3.88 (s, 3H), 3.05 (t, J=5.0 Hz, 4H). MS (ESI) m/z [M+H]⁺ calcd. for C₂₂H₂₂N₁O₀, 443.2; found, 443.2; HPLC purity: 254 nm: 99.6%.

### Example 226: Synthesis of Exemplary Compound 302

### 3-(4-(5-(furan-3-ylmethyl)pyrimidin-2-yl)piperazin-1-yl)-6-(1-methyl-1H-pyrazol-4-yl)pyrazolo[1,5-a]pyridine (Compound 302)

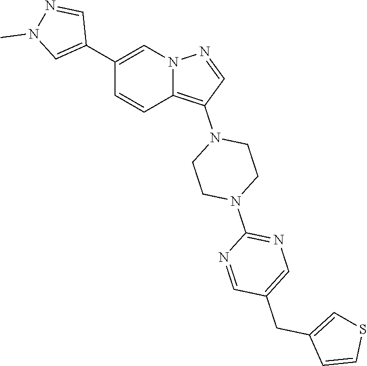 [SMILES: Cn1cc(-c2ccc3c(N4CCN(c5ncc(Cc6ccsc6)cn5)CC4)cnn3c2)cn1]

### Step 1. Preparation of 2-chloro-5-(thiophen-3-ylmethyl)pyrimidine

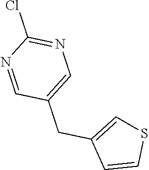 [SMILES: Clc1ncc(Cc2ccsc2)cn1]

To a solution of 2-chloro-5-(chloromethyl)pyrimidine (1.0 g, 6.14 mmol) and thiophen-3-ylboronic acid (0.47 g, 3.68 mmol) in dioxane (20 mL) and H₂O (1 mL) were added Pd(dppf)Cl₂ (0.9 g, 1.23 mmol) and K₃PO₄ (2.6 g, 12.27 mmol). The reaction was stirred for 16 h at 60° C. under nitrogen. The mixture cooled to rt then concentrated under reduced pressure. The residue was diluted with H₂O and the resulting mixture was extracted with EtOAc (3×80 mL). The combined organic layers were washed with brine (10 mL) and dried over anhydrous Na₂SO₄. After filtration, the filtrate was concentrated under reduced pressure. The residue was purified by silica gel column chromatography eluting with PE/EtOAc (1:1) to afford 200 mg (16%) of 2-chloro-5-(thiophen-3-ylmethyl)pyrimidine as a yellow solid: ¹H NMR (400 MHZ, DMSO-d₆) δ (ppm) 8.69 (s, 2H), 7.50 (dd, J=4.9, 2.9 Hz, 1H), 7.33-7.22 (m, 1H), 7.04 (d, J=5.0 Hz, 1H), 4.00 (s, 2H). MS (ESI) m/z [M+H]⁺ calcd. for C₉H₇ClN₂S, 211.0; found, 211.1.

### Step 2. 6-(1-methyl-1H-pyrazol-4-yl)-3-(4-(5-(thiophen-3-ylmethyl)pyrimidin-2-yl)piperazin-1-yl)pyrazolo[1,5-a]pyridine (Compound 302)

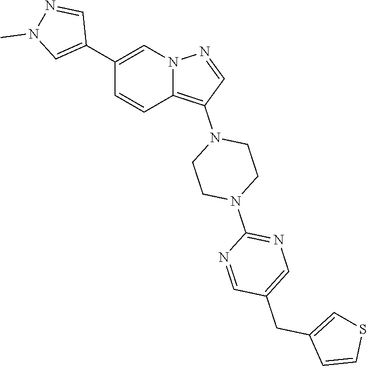 [SMILES: Cn1cc(-c2ccc3c(N4CCN(c5ncc(Cc6ccsc6)cn5)CC4)cnn3c2)cn1]

To a solution of 2-chloro-5-(thiophen-3-ylmethyl)pyrimidine (100 mg, 0.48 mmol, as prepared in the previous step) and 6-(1-methyl-1H-pyrazol-4-yl)-3-(piperazin-1-yl)pyrazolo[1,5-a]pyridine (Compound 12) (160.8 mg, 0.57 mmol) in IPA (3 mL) was added DIPEA (122.7 mg, 0.95 mmol). The reaction was stirred for 4 h at 90° C. under nitrogen. The mixture was cooled to rt then concentrated under reduced pressure. The crude product was purified by Prep-HPLC to afford 85.5 mg (39%) of 6-(1-methyl-1H-pyrazol-4-yl)-3-(4-(5-(thiophen-3-ylmethyl)pyrimidin-2-yl)piperazin-1-yl)pyrazolo[1,5-a]pyridine (Compound 302) as a light yellow solid. ¹H NMR (400 MHZ, DMSO-d₆) δ (ppm) 8.83 (s, 1H), 8.31 (s, 2H), 8.23 (s, 1H), 7.97 (s, 1H), 7.78 (s, 1H), 7.68 (d, J=9.2 Hz, 1H), 7.47 (dd, J=4.9, 2.9 Hz, 1H), 7.32 (dd, J=9.3, 1.5 Hz, 1H), 7.19 (d, J=3.0 Hz, 1H), 7.00 (d, J=5.0 Hz, 1H), 3.93-3.84 (m, 7H), 3.79 (s, 2H), 3.04 (t, J=5.0 Hz, 4H). MS (ESI) m/z [M+H]⁺ calcd. for C₂₄H₂₄N₈S, 457.2; found, 457.1; HPLC purity: 254 nm: 99.0%.

### Example 227: Synthesis of Exemplary Compound 303

### 3-(4-(5-benzylpyrazin-2-yl)piperazin-1-yl)-6-(1-methyl-1H-pyrazol-4-yl)pyrazolo[1,5-a]pyridine (Compound 303)

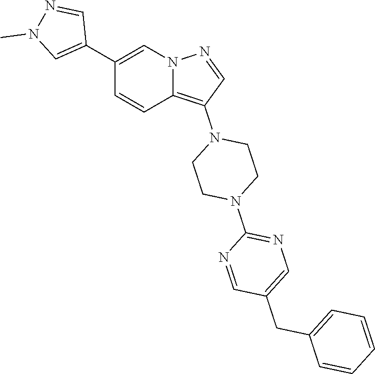 [SMILES: Cn1cc(-c2ccc3c(N4CCN(c5ncc(Cc6ccccc6)cn5)CC4)cnn3c2)cn1]

### Step 1. Preparation of 2-chloro-5-(chloromethyl)pyrazine

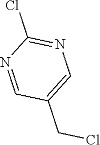

A mixture of (5-chloropyrazin-2-yl)methanol (1.00 g, 6.92 mmol) and thionyl chloride (10 mL) was stirred for 16 h at rt under nitrogen then was concentrated under reduced pressure. The residue was diluted with water and the resulting mixture was extracted with EtOAc (3×50 mL). The combined organic layers were washed with brine (10 mL) and dried over anhydrous Na₂SO₄. After filtration, the filtrate was concentrated under reduced pressure. The residue was purified by silica gel column chromatography eluting with PE/EtOAc (3:1) to afford 0.80 g (71%) of 2-chloro-5-(chloromethyl)pyrazine as a light yellow solid. ¹H NMR (400 MHZ, DMSO-d₆) δ (ppm) 8.82 (d, J=1.3 Hz, 1H), 8.67 (d, J=1.3 Hz, 1H), 4.88 (s, 2H). MS (ESI) m/z [M+H]⁺ calcd. for C₅H₄Cl₂N₂, 163.0; found, 163.1.

### Step 2. Preparation of 2-benzyl-5-chloropyrazine

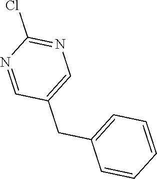 [SMILES: Clc1ncc(Cc2ccccc2)cn1]

To a solution of 2-chloro-5-(chloromethyl)pyrazine (800 mg, 4.90 mmol, as prepared in the previous step) and phenylboronic acid (53.9 mg, 0.44 mmol) in toluene (10 mL) and H₂O (1 mL) were added K₃PO₄ (2.08 g, 9.82 mmol), (tBu)₃P Pd G3 (561.5 mg, 0.98 mmol) and (tBu)₃P·HBF₄ (284.8 mg, 0.98 mmol). The reaction was stirred for 16 h at 90° C. under nitrogen. The mixture was cooled to rt then concentrated under reduced pressure. The residue was diluted with H₂O then was extracted with EtOAc (3×50 mL). The combined organic layers were washed with brine (10 mL) and dried over anhydrous Na₂SO₄. After filtration, the filtrate was concentrated under reduced pressure. The residue was purified by Prep-TLC (DCM/MeOH 10:1) to afford 340.0 mg (34%) of 2-benzyl-5-chloropyrazine as a yellow solid. ¹H NMR (400 MHZ, DMSO-d₆) δ (ppm) 9.38-9.24 (m, 1H), 8.99-8.80 (m, 1H), 8.30-8.08 (m, 2H), 7.72-7.44 (m, 3H), 5.10-4.81 (m, 2H). MS (ESI) m/z [M+H]⁺ calcd. for C₁₁H₉ClN₂, 205.0; found, 205.1.

### Step 3. 3-(4-(5-benzylpyrazin-2-yl)piperazin-1-yl)-6-(1-methyl-1H-pyrazol-4-yl)pyrazolo[1,5-a]pyridine (Compound 303)

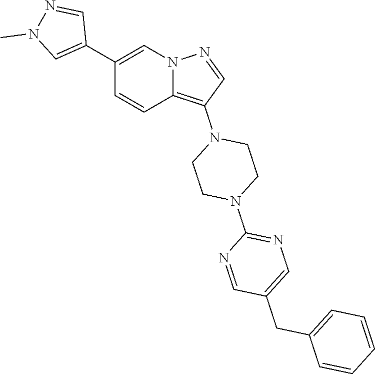 [SMILES: Cn1cc(-c2ccc3c(N4CCN(c5ncc(Cc6ccccc6)cn5)CC4)cnn3c2)cn1]

To a solution of 2-benzyl-5-chloropyrazine (100 mg, 0.49 mmol, as prepared in the previous step) and 6-(1-methyl-1H-pyrazol-4-yl)-3-(piperazin-1-yl)pyrazolo[1,5-a]pyridine (Compound 12) (165.6 mg, 0.59 mmol) in IPA (5 mL) was added DIPEA (126.3 mg, 0.98 mmol). The reaction was stirred for 16 h at 90° C. under nitrogen then concentrated under reduced pressure. The residue was treated with water then extracted with EtOAc (3×50 mL). The combined organic layers were washed with brine (10 mL), dried over anhydrous Na₂SO₄, and filtered. The filtrate was concentrated under reduced pressure. The crude product was purified by Prep-HPLC to afford 61.9 mg (28%) of 3-(4-(5-benzylpyrazin-2-yl)piperazin-1-yl)-6-(1-methyl-1H-pyrazol-4-yl)pyrazolo[1,5-a]pyridine (Compound 303) as a white solid. ¹H NMR (400 MHZ, DMSO-d₆) δ (ppm) 9.20 (s, 1H), 8.83-8.75 (m, 2H), 8.21 (s, 1H), 8.18-8.10 (m, 2H), 7.96 (s, 1H), 7.74 (s, 1H), 7.62 (d, J=9.2 Hz, 1H), 7.59-7.46 (m, 3H), 7.29 (dd, J=9.2, 1.5 Hz, 1H), 3.86 (s, 3H), 3.80 (s, 2H), 3.05 (t, J=4.7 Hz, 4H), 2.69 (t, J=4.7 Hz, 4H). MS (ESI) m/z [M+H]⁺ calcd. for C₂₆H₂₆N₈,451.2; found, 451.2; HPLC purity: 254 nm: 99.4%.

### Example 228: Synthesis of Exemplary Compound 304

### tert-butyl 4-(6-(tetrahydrofuran-3-yl)pyrazolo[1,5-a]pyridin-3-yl)piperazine-1-carboxylate (Compound 304)

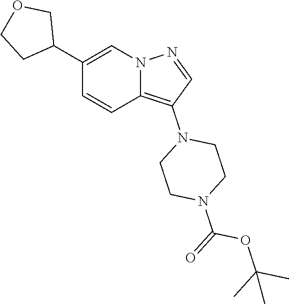 [SMILES: CC(C)(C)OC(=O)N1CCN(c2cnn3cc(C4CCOC4)ccc23)CC1]

### Step 1. Preparation of 6-(2,5-dihydrofuran-3-yl)pyrazolo[1,5-a]pyridine

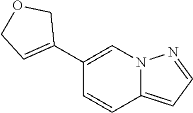 [SMILES: C1=C(c2ccc3ccnn3c2)COC1]

To a solution of 6-bromopyrazolo[1,5-a]pyridine (1.00 g, 5.08 mmol) and 2-(2,5-dihydrofuran-3-yl)-4,4,5,5-tetramethyl-1,3,2-dioxaborolane (1.19 g, 6.09 mmol) in dioxane (20 mL) and H₂O (0.2 mL) were added K₂CO₃ (1.40 g, 10.15 mmol) and Pd(dppf)Cl₂ (0.74 g, 1.02 mmol). The reaction was stirred for 16 h at 90° C. under nitrogen. The mixture was allowed to cool to rt then concentrated under reduced pressure. The residue was diluted with water and the resulting mixture was extracted with EtOAc (3×100 mL). The combined organic layers were washed with brine (10 mL), dried over anhydrous Na₂SO₄. After filtration, the filtrate was concentrated under reduced pressure. The residue was purified by silica gel column chromatography eluting with DCM/MeOH (10:1) to afford 810 mg (86%) of 6-(2,5-dihydrofuran-3-yl)pyrazolo[1,5-a]pyridine as a white solid. ¹H NMR (400 MHZ, DMSO-d₆) δ (ppm) 8.55 (s, 1H), 8.01 (d, J=2.2 Hz, 1H), 7.70 (d, J=9.2 Hz, 1H), 7.52 (dd, J=9.3, 1.6 Hz, 1H), 6.65-6.56 (m, 2H), 4.99-4.83 (m, 2H), 4.78-4.77 (m, 2H). MS (ESI) m/z [M+H]⁺ calcd. for C₁₁H₁₀N₂O, 187.1; found, 187.2.

### Step 2. Preparation of 6-(tetrahydrofuran-3-yl)pyrazolo[1,5-a]pyridine

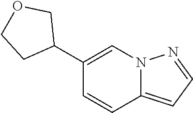 [SMILES: c1cc2ccc(C3CCOC3)cn2n1]

To a solution of 6-(2,5-dihydrofuran-3-yl)pyrazolo[1,5-a]pyridine (800 mg, 4.30 mmol, as prepared in the previous step) in 15 mL MeOH was added 10% Pd/C (457.2 mg, 0.43 mmol) under nitrogen. The reaction was stirred at rt for 4 h under hydrogen atmosphere then filtered through a Celite pad and concentrated under reduced pressure. The residue was purified by Prep-TLC (DCM/MeOH 10:1) to afford 600.0 mg (74%) of 6-(oxolan-3-yl)pyrazolo[1,5-a]pyridine as a white solid. ¹H NMR (400 MHZ, DMSO-d₆) δ (ppm) 8.56 (s, 1H), 7.94 (d, J=2.3 Hz, 1H), 7.66 (d, J=9.1 Hz, 1H), 7.18 (dd, J=9.1, 1.6 Hz, 1H), 6.59-6.50 (m, 1H), 4.07-3.92 (m, 2H), 3.85-3.76 (m, 1H), 3.64-3.56 (m, 1H), 3.49-3.37 (m, 1H), 2.37-2.22 (m, 1H), 2.03-1.93 (m, 1H). MS (ESI) m/z [M+H]⁺ calcd. for C₁₁H₁₂N₂O, 189.1; found, 189.3.

### Step 3. Preparation of 3-bromo-6-(1,5-dimethyl-1H-pyrazol-4-yl)pyrazolo[1,5-a]pyridine

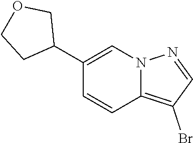 [SMILES: Brc1cnn2cc(C3CCOC3)ccc12]

To a solution of 6-(tetrahydrofuran-3-yl)pyrazolo[1,5-a]pyridine (600 mg, 3.19 mmol, as prepared in the previous step) in DCM (10 mL) was added NBS (624.1 mg, 3.51 mmol). The reaction was stirred for 4 h at rt under nitrogen then concentrated under reduced pressure. The residue was diluted with water then extracted with DCM (3×50 mL). The combined organic layers were washed with brine (10 mL), dried over anhydrous Na₂SO₄. After filtration, the filtrate was concentrated under reduced pressure. The residue was purified by Prep-TLC (DCM/MeOH 5:1) to afford 440 mg (52%) of 3-bromo-6-(oxolan-3-yl)pyrazolo[1,5-a]pyridine as a yellow solid. ¹H NMR (400 MHZ, DMSO-d₆) δ (ppm) 8.63 (s, 1H), 8.10 (s, 1H), 7.58-7.51 (m, 1H), 7.33 (dd, J=9.2, 1.5 Hz, 1H), 4.07-3.93 (m, 2H), 3.85-3.74 (m, 1H), 3.67-3.55 (m, 1H), 3.51-3.40 (m, 1H), 2.40-2.25 (m, 1H), 2.03-1.89 (m, 1H). MS (ESI) m/z [M+H]⁺ calcd. for C₁₁H₁₁BrN₂O, 267.0; found, 267.1.

### Step 4. tert-butyl 4-(6-(tetrahydrofuran-3-yl)pyrazolo[1,5-a]pyridin-3-yl)piperazine-1-carboxylate (Compound 304)

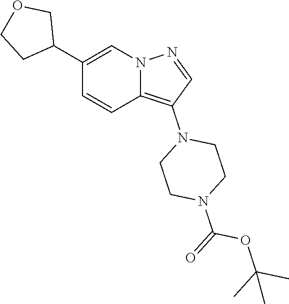 [SMILES: CC(C)(C)OC(=O)N1CCN(c2cnn3cc(C4CCOC4)ccc23)CC1]

To a solution of 3-bromo-6-(tetrahydrofuran-3-yl)pyrazolo[1,5-a]pyridine (400 mg, 1.50 mmol, as prepared in the previous step) and tert-butyl piperazine-1-carboxylate (1.39 g, 7.49 mmol) in dioxane (5 mL) and tBuOH (5 mL) were added tBuXPhos Pd G1 (205.7 mg, 0.30 mmol) and KOtBu (336.1 mg, 2.99 mmol). The reaction stirred for 16 h at 90° C. under nitrogen. The mixture was allowed to cool to rt then concentrated under reduced pressure. The residue was diluted with water and the resulting mixture was extracted with EtOAc (3×100 mL). The combined organic layers were washed with brine (10 mL), dried over anhydrous Na₂SO₄, and filtered. The filtrate was concentrated under reduced pressure then the residue was purified by silica gel column chromatography eluting with DCM/MeOH (10:1) to afford crude product. The crude product was purified by Prep-HPLC to afford 143.6 mg (26%) of tert-butyl 4-(6-(tetrahydrofuran-3-yl)pyrazolo[1,5-a]pyridin-3-yl)piperazine-1-carboxylate (Compound 304) as a light yellow solid. ¹H NMR (400 MHZ, DMSO-d₆) δ (ppm) 8.41 (s, 1H), 7.75 (s, 1H), 7.59 (d, J=9.2 Hz, 1H), 7.05 (dd, J=9.2 Hz, 1H), 4.06-3.91 (m, 2H), 3.79 (q, J=7.8 Hz, 1H), 3.62-3.55 (m, 1H), 3.49 (t, J=9.9 Hz, 4H), 3.45-3.34 (m, 1H), 2.91 (t, J=5.1 Hz, 4H), 2.36-2.25 (m, 1H), 2.02-1.89 (m, 1H), 1.42 (s, 9H). MS (ESI) m/z [M+H]⁺ calcd. for C₂₀H₂₈N₄O₃, 373.3; found, 373.2; HPLC purity: 254 nm: 99.7%.

### Example 229: Synthesis of Exemplary Compound 305

### 3-(2-(5-benzylpyrimidin-2-yl)ethyl)-6-(1-methyl-1H-pyrazol-4-yl)pyrazolo[1,5-a]pyridine (Compound 305)

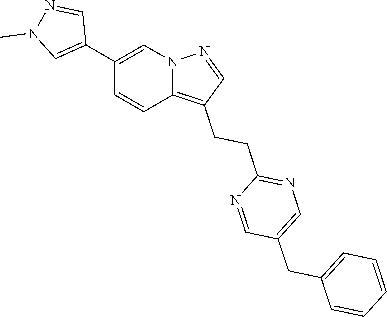 [SMILES: Cn1cc(-c2ccc3c(CCc4ncc(Cc5ccccc5)cn4)cnn3c2)cn1]

### Step 1. Preparation of 6-(1-methyl-1H-pyrazol-4-yl)-3-((trimethylsilyl)ethynyl)pyrazolo[1,5-a]pyridine

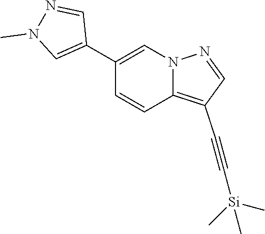

To a solution of 4-{3-bromopyrazolo[1,5-a]pyridin-6-yl}-1-methylpyrazole (Compound S9) (1.00 g, 3.61 mmol) and trimethylsilylacetylene (0.80 g, 8.12 mmol) in ACN (10 mL) was added with (PPh₃)₂PdCl₂ (0.76 g, 1.08 mmol), CuI (0.10 g, 0.54 mmol) and Et₃N (1.83 g, 18 mmol) under nitrogen. The reaction was stirred for 16 h at 70° C. under nitrogen then concentrated under reduced pressure. The residue was purified by silica gel column chromatography eluting with PE/EtOAc (2:1) to afford 640 mg (54%) of 6-(1-methyl-1H-pyrazol-4-yl)-3-((trimethylsilyl)ethynyl)pyrazolo[1,5-a]pyridine as a yellow solid. ¹H NMR (400 MHZ, DMSO-d₆) δ (ppm) 9.08 (s, 1H), 8.29 (s, 1H), 8.20 (d, J=1.6 Hz, 1H), 8.03 (s, 1H), 7.67 (s, 2H), 3.88 (s, 3H), 0.26 (s, 9H). MS (ESI) m/z [M+H]⁺ calcd. for C₁₆H₁₈N₄Si, 295.1; found, 295.2.

### Step 2. Preparation of 3-ethynyl-6-(1-methyl-1H-pyrazol-4-yl)pyrazolo[1,5-a]pyridine

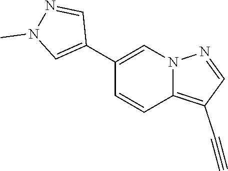 [SMILES: C#Cc1cnn2cc(-c3cnn(C)c3)ccc12]

To a solution of 6-(1-methyl-1H-pyrazol-4-yl)-3-((trimethylsilyl)ethynyl)pyrazolo[1,5-a]pyridine (640 mg, 2.17 mmol, as prepared in the previous step) in MeOH (15.0 mL) was added K₂CO₃ (901 mg, 6.52 mmol). The solution was stirred for 1 h at rt. The mixture was diluted with water (100 mL) and extracted with EtOAc (2×100 mL). The combined organic layers were dried over anhydrous Na₂SO₄. After filtration, the filtrate was concentrated under reduced pressure. The residue was purified by silica gel column chromatography eluting with PE/EtOAc (1:1) to afford 450 mg (84%) of 3-ethynyl-6-(1-methyl-1H-pyrazol-4-yl)pyrazolo[1,5-a]pyridine as a yellow solid. ¹H NMR (400 MHZ, DMSO-d₆) δ (ppm) 9.08 (s, 1H), 8.29 (s, 1H), 8.20 (s, 1H), 8.03 (s, 1H), 7.75-7.50 (m, 2H), 4.29 (s, 1H), 3.88 (s, 3H). MS (ESI) m/z [M+H]⁺ calcd. for C₁₃H₁₀N₄, 223.1; found, 223.2.

### Step 3. Preparation of 3-((5-benzylpyrimidin-2-yl)ethynyl)-6-(1-methyl-1H-pyrazol-4-yl)pyrazolo[1,5-a]pyridine

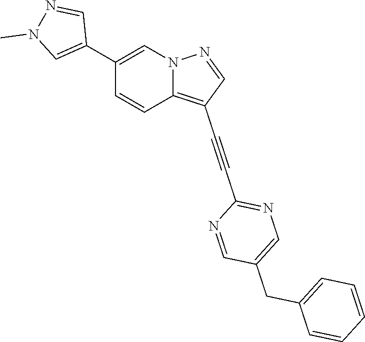 [SMILES: Cn1cc(-c2ccc3c(C#Cc4ncc(Cc5ccccc5)cn4)cnn3c2)cn1]

To a solution of 3-ethynyl-6-(1-methyl-1H-pyrazol-4-yl)pyrazolo[1,5-a]pyridine (70.0 mg, 0.32 mmol, as prepared in the previous step) and 5-benzyl-2-chloropyrimidine (Compound S88) (52.0 mg, 0.25 mmol) in DMF (2 mL) was added with tBuXPhos Pd G3 (25.0 mg, 0.032 mmol), tBuXPhos (13.4 mg, 0.032 mmol), CuI (3 mg, 0.016 mmol) and Et₃N (63.8 mg, 0.63 mmol) under nitrogen. The reaction was stirred for 16 h at 90° C. under nitrogen then concentrated under reduced pressure. The residue was purified by Prep-TLC (EtOAc) to afford 45.0 mg (22%) of 3-((5-benzylpyrimidin-2-yl)ethynyl)-6-(1-methyl-1H-pyrazol-4-yl)pyrazolo[1,5-a]pyridine as a yellow oil; MS (ESI) m/z [M+H]⁺ calcd. for C₂₄H₁₈N₆, 391.2; found, 391.3.

### Step 4. Preparation of 3-(2-(5-benzylpyrimidin-2-yl)ethyl)-6-(1-methyl-1H-pyrazol-4-yl)pyrazolo[1,5-a]pyridine (Compound 305)

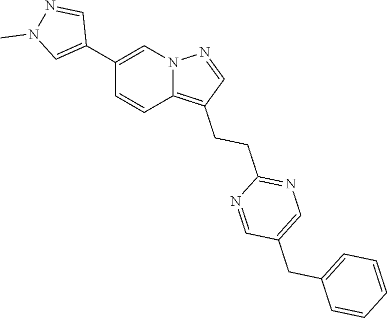 [SMILES: Cn1cc(-c2ccc3c(CCc4ncc(Cc5ccccc5)cn4)cnn3c2)cn1]

To a solution of 3-((5-benzylpyrimidin-2-yl)ethynyl)-6-(1-methyl-1H-pyrazol-4-yl)pyrazolo[1,5-a]pyridine (45 mg, 0.115 mmol, as prepared in the previous step) in MeOH (4.0 mL) was added Pd/C (6.2 mg, 0.058 mmol). The reaction was stirred for 4 h at rt under hydrogen atmosphere. The resulting mixture was filtered, and the filter cake was washed with MeOH (2×2 mL). The filtrate was concentrated under reduced pressure. The crude product was purified by Prep-HPLC to afford 4.7 mg (10%) of 3-(2-(5-benzylpyrimidin-2-yl)ethyl)-6-(1-methyl-1H-pyrazol-4-yl)pyrazolo[1,5-a]pyridine (Compound 305) as a white solid. ¹H NMR (400 MHZ, DMSO-d₆) δ (ppm) 8.85 (s, 1H), 8.63 (s, 2H), 8.21 (s, 1H), 7.96 (d, J=0.8 Hz, 1H), 7.74 (s, 1H), 7.54 (dd, J=9.3, 0.9 Hz, 1H), 7.37-7.17 (m, 6H), 3.95 (s, 2H), 3.88 (s, 3H), 3.19 (s, 4H). MS (ESI) m/z [M+H]⁺ calcd. for C₂₄H₂₂N₆, 395.2; found, 395.2; HPLC purity: 254 nm: 98.9%.

### Example 230: Synthesis of Exemplary Compound 306

### 2-benzyl-5-(4-(6-(1-methyl-1H-pyrazol-4-yl)pyrazolo[1,5-a]pyridin-3-yl)piperazin-1-yl)-1,3,4-thiadiazole (Compound 306)

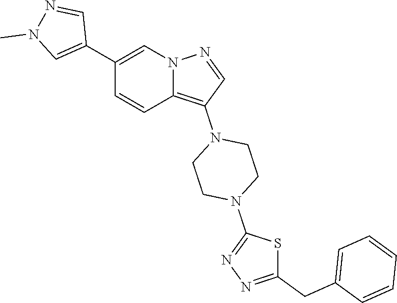 [SMILES: Cn1cc(-c2ccc3c(N4CCN(c5nnc(Cc6ccccc6)s5)CC4)cnn3c2)cn1]

### Step 1. Preparation of 2-bromo-5-(4-(6-(1-methyl-1H-pyrazol-4-yl)pyrazolo[1,5-a]pyridin-3-yl)piperazin-1-yl)-1,3,4-thiadiazole

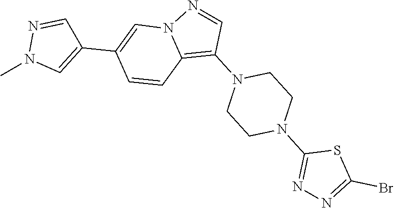 [SMILES: Cn1cc(-c2ccc3c(N4CCN(c5nnc(Br)s5)CC4)cnn3c2)cn1]

To a solution of 2,5-dibromo-1,3,4-thiadiazole (450 mg, 1.85 mmol), DIPEA (477 mg, 3.69 mmol) in IPA (4.0 mL) was added 1-[6-(1-methylpyrazol-4-yl)pyrazolo[1,5-a]pyridin-3-yl]piperazine (Compound 12) (208 mg, 0.74 mmol). The reaction was stirred for 3 h at 90° C. The precipitated solids were collected by filtration and washed with ACN (2×2 mL) to afford 270 mg (30%) of 2-bromo-5-(4-(6-(1-methyl-1H-pyrazol-4-yl)pyrazolo[1,5-a]pyridin-3-yl)piperazin-1-yl)-1,3,4-thiadiazole as a light reddish brown solid. ¹H NMR (400 MHz, DMSO-d₆) δ (ppm) 8.84 (s, 1H), 8.24 (s, 1H), 7.98 (d, J=0.8 Hz, 1H), 7.81 (s, 1H), 7.69 (dd, J=9.3, 1.0 Hz, 1H), 7.35 (dd, J=9.2, 1.5 Hz, 1H), 3.87 (s, 3H), 3.72-3.55 (m, 4H), 3.21-3.03 (m, 4H). MS (ESI) m/z [M+H]⁺ calcd. for C₁₇H₁₇BrN₈S, 445.0; found, 445.1.

### Step 2. Preparation of 2-benzyl-5-(4-(6-(1-methyl-1H-pyrazol-4-yl)pyrazolo[1,5-a]pyridin-3-yl)piperazin-1-yl)-1,3,4-thiadiazole (Compound 306)

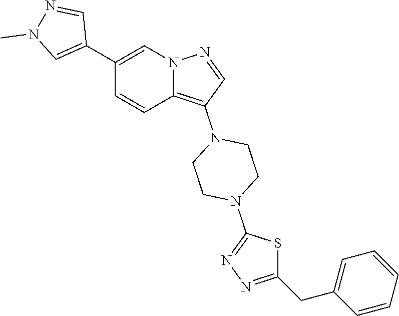 [SMILES: Cn1cc(-c2ccc3c(N4CCN(c5nnc(Cc6ccccc6)s5)CC4)cnn3c2)cn1]

To a solution of 2-bromo-5-(4-(6-(1-methyl-1H-pyrazol-4-yl)pyrazolo[1,5-a]pyridin-3-yl)piperazin-1-yl)-1,3,4-thiadiazole (300 mg, 0.67 mmol, as prepared in the previous step) and potassium benzyltrifluoroborate (667 mg, 3.37 mmol) in dioxane (10.0 mL) and H₂O (2 mL) was added K₃PO₄ (286 mg, 1.35 mmol), and Pd-PEPPSI-IPent^{Cl} 2-methylpyridine (56.7 mg, 0.07 mmol). The reaction was stirred for 40 h at 90° C. under nitrogen then concentrated under reduced pressure. The crude product (50 mg) was purified by Prep-HPLC to afford 11.1 mg (4%) of 2-benzyl-5-(4-(6-(1-methyl-1H-pyrazol-4-yl)pyrazolo[1,5-a]pyridin-3-yl)piperazin-1-yl)-1,3,4-thiadiazole (Compound 306) as a yellow solid. ¹H NMR (400 MHZ, DMSO-d₆) δ (ppm) 8.83 (s, 1H), 8.23 (s, 1H), 7.97 (d, J=0.8 Hz, 1H), 7.79 (s, 1H), 7.67 (dd, J=9.3, 1.0 Hz, 1H), 7.41-7.22 (m, 6H), 4.25 (s, 2H), 3.87 (s, 3H), 3.59 (t, J=5.1 Hz, 4H), 3.14-3.02 (m, 4H). MS (ESI) m/z [M+H]⁺ calcd. for C₂₄H₂₄N₈S, 457.2; found, 457.1; HPLC purity: 254 nm: 98.5%.

### Example 231: Synthesis of Exemplary Compound 318

(R)-1-phenylethyl 4-(6-(1-methyl-1H-pyrazol-4-yl)pyrazolo[1,5-a]pyridin-3-yl)-1,4-diazepane-1-carboxylate (Compound 318)

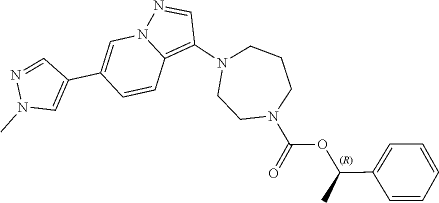

### Step 1. Preparation of 3-(1,4-diazepan-1-yl)-6-(1-methyl-1H-pyrazol-4-yl)pyrazolo[1,5-a]pyridine hydrochloride salt

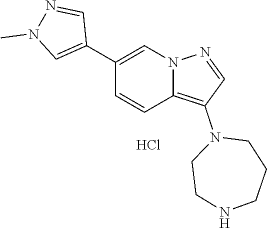 [SMILES: Cn1cc(-c2ccc3c(N4CCCNCC4)cnn3c2)cn1 ; Cl]

To a solution of tert-butyl 4-(6-(1-methyl-1H-pyrazol-4-yl)pyrazolo[1,5-a]pyridin-3-yl)-1,4-diazepane-1-carboxylate (Compound 40) (245 mg, 0.62 mmol) in dioxane (6.0 mL) was added 4M HCl in dioxane (12.0 mL). The resulting mixture was stirred for 1 h at rt. The mixture was concentrated under reduced pressure to afford 290 mg (87%) of 3-(1,4-diazepan-1-yl)-6-(1-methyl-1H-pyrazol-4-yl)pyrazolo[1,5-a]pyridine hydrochloride salt as a light yellow solid. MS (ESI) m/z [M+H]⁺ calcd. for C₁₆H₂₀N₆, 297.2; found, 297.3.

### Step 2. Preparation of (R)-1-phenylethyl 4-(6-(1-methyl-1H-pyrazol-4-yl)pyrazolo[1,5-a]pyridin-3-yl)-1,4-diazepane-1-carboxylate (Compound 318)

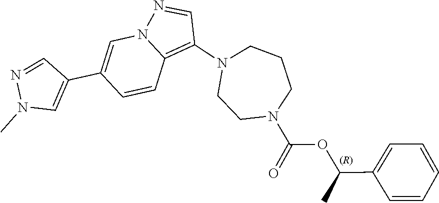

To a solution of 3-(1,4-diazepan-1-yl)-6-(1-methyl-1H-pyrazol-4-yl)pyrazolo[1,5-a]pyridine (50 mg, 0.17 mmol, as prepared in the previous step), (R)-1-phenylethan-1-ol (25 mg, 0.20 mmol) and pyridine (20 mg, 0.25 mmol) in DCM (2.0 mL) was added triphosgene (15 mg, 0.05 mmol) at 0° C. The resulting mixture was stirred for 1 h at 0° C. The resulting mixture was concentrated under reduced pressure. The residue was purified by Prep-TLC (EtOAc). The crude product was purified by Prep-HPLC to afford 54.0 mg (72%) of (R)-1-phenylethyl 4-(6-(1-methyl-1H-pyrazol-4-yl)pyrazolo[1,5-a]pyridin-3-yl)-1,4-diazepane-1-carboxylate (Compound 318) as a yellow solid. ¹H NMR (400 MHZ, DMSO-d₆) δ (ppm) 8.76-8.71 (m, 1H), 8.20 (s, 1H), 7.95 (s, 1H), 7.66 (d, J=1.2 Hz, 1H), 7.60-7.53 (m, 1H), 7.34 (d, J=4.3 Hz, 2H), 7.31-7.18 (m, 4H), 5.75-5.59 (m, 1H), 3.87 (s, 3H), 3.75-3.49 (m, 3H), 3.49-3.41 (m, 2H), 3.41-3.33 (m, 2H), 3.31-3.22 (m, 1H), 2.01-1.73 (m, 2H), 1.50-1.27 (m, 3H); MS (ESI) m/z [M+H]⁺ calcd. for C₂₅H₂₈N₆O₂, 445.2; found, 445.1; LCMS purity: 254 nm: 99.4%.

### Example 232: Synthesis of Exemplary Compound 319

### 3-(4-(5-benzylpyrimidin-2-yl)piperazin-1-yl)-6-(pyridin-4-yl)pyrazolo[1,5-a]pyridine (Compound 319)

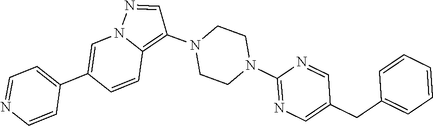 [SMILES: c1ccc(Cc2cnc(N3CCN(c4cnn5cc(-c6ccncc6)ccc45)CC3)nc2)cc1]

### Step 1. 3-(piperazin-1-yl)-6-(pyridin-4-yl)pyrazolo[1,5-a]pyridine

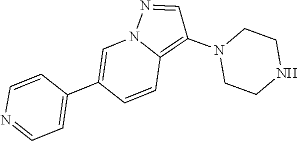 [SMILES: c1cc(-c2ccc3c(N4CCNCC4)cnn3c2)ccn1]

To a solution of tert-butyl 4-[6-(pyridin-4-yl)pyrazolo[1,5-a]pyridin-3-yl]piperazine-1-carboxylate (Compound 218) (200 mg, 0.52 mmol) in DCM (5 mL) was added TFA (1 mL). After stirring for 1 h at rt under nitrogen, the mixture was basified to pH 8 with sat. aq. NaHCO₃. The mixture was extracted with DCM (3×30 mL), dried over anhydrous Na₂SO₄, and filtered. The filtrate was concentrated under reduced pressure to afford 110 mg (75%) of 1-[6-(pyridin-4-yl)pyrazolo[1,5-a]pyridin-3-yl]piperazine as a yellow solid. ¹H NMR (400 MHZ, DMSO-d₆) δ (ppm) 9.13 (s, 1H), 8.70-8.56 (m, 2H), 7.88 (s, 1H), 7.85-7.81 (m, 2H), 7.75 (d, J=9.4 Hz, 1H), 7.49 (d, J=9.4 Hz, 1H), 2.96-2.87 (m, 8H), 1.24 (s, 1H); MS (ESI) m/z [M+H]⁺ calcd. for C₁₆H₁₇N₅, 280.1; found, 280.1.

### Step 2. 3-(4-(5-benzylpyrimidin-2-yl)piperazin-1-yl)-6-(pyridin-4-yl)pyrazolo[1,5-a]pyridine (Compound 319)

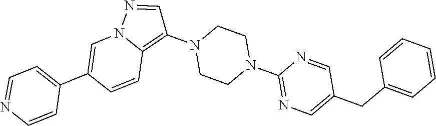 [SMILES: c1ccc(Cc2cnc(N3CCN(c4cnn5cc(-c6ccncc6)ccc45)CC3)nc2)cc1]

To a solution of 1-[6-(pyridin-4-yl)pyrazolo[1,5-a]pyridin-3-yl]piperazine (110 mg, 0.39 mmol, as prepared in the previous step) and 5-benzyl-2-chloropyrimidine (Compound S88) (120.9 mg, 0.59 mmol) in IPA (10 mL) was added DIPEA (101.8 mg, 0.78 mmol). After stirring overnight at 105° C. under nitrogen atmosphere, the mixture was allowed to cool to rt. The resulting mixture was filtered, the filter cake was washed with EtOAc (3×30 mL). The filtrate was concentrated under reduced pressure. The crude product was purified by Prep-HPLC to afford 60.8 mg (34%) of 3-(4-(5-benzylpyrimidin-2-yl)piperazin-1-yl)-6-(pyridin-4-yl)pyrazolo[1,5-a]pyridine (Compound 319) as a yellow solid. ¹H NMR (400 MHZ, DMSO-d₆) δ (ppm) 9.15 (s, 1H), 8.69-8.60 (m, 2H), 8.32 (s, 2H), 7.94 (s, 1H), 7.89-7.77 (m, 3H), 7.54 (d, J=9.4 Hz, 1H), 7.35-7.13 (m, 5H), 3.90 (t, J=4.9 Hz, 4H), 3.80 (s, 2H), 3.07 (t, J=4.9 Hz, 4H); MS (ESI) m/z [M+H]⁺ calcd. for C₂₇H₂₅N₇, 448.1; found, 448.1.LCMS purity: 254 nm: 98.9%.

### Example 233: Synthesis of Exemplary Compound 320

### 3-(4-(5-benzylpyrimidin-2-yl)piperazin-1-yl)-6-(pyridin-3-yl)pyrazolo[1,5-a]pyridine (Compound 320)

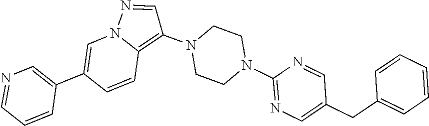 [SMILES: c1ccc(Cc2cnc(N3CCN(c4cnn5cc(-c6cccnc6)ccc45)CC3)nc2)cc1]

### Step 1. Preparation of 3-(piperazin-1-yl)-6-(pyridin-3-yl)pyrazolo[1,5-a]pyridine

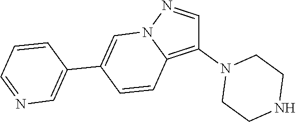 [SMILES: c1cncc(-c2ccc3c(N4CCNCC4)cnn3c2)c1]

To a solution of tert-butyl 4-(6-(pyridin-3-yl)pyrazolo[1,5-a]pyridin-3-yl)piperazine-1-carboxylate (Compound 217) (400.0 mg, 1.05 mmol) in DCM (3 mL) were added TFA (1 mL). After stirring for 4 h at rt, the mixture was neutralized to pH 8 with sat. aq. NaHCO₃. The resulting mixture was extracted with EtOAc (3×40 mL). The combined organic layers were washed with brine (10 mL), dried over anhydrous Na₂SO₄, and filtered. The filtrate was concentrated under reduced pressure. The residue was purified by Prep-TLC (DCM/MeOH 1:1) to afford 150 mg (51%) 3-(piperazin-1-yl)-6-(pyridin-3-yl)pyrazolo[1,5-a]pyridine as a yellow solid: ¹H NMR (400 MHz, DMSO-d₆) δ (ppm) 9.02-8.93 (m, 2H), 8.58 (dd, J=4.7, 1.6 Hz, 1H), 8.17 (d, J=8.1 Hz, 1H), 7.85-7.80 (m, 1H), 7.72 (d, J=9.2 Hz, 1H), 7.54-7.39 (m, 2H), 3.11-2.65 (m, 8H), 2.18 (s, 1H).; MS (ESI) m/z [M+H]⁺ calcd. for C₁₆H₁₇N₅, 280.1; found, 280.1.

### Step 2. 3-(4-(5-benzylpyrimidin-2-yl)piperazin-1-yl)-6-(pyridin-3-yl)pyrazolo[1,5-a]pyridine (Compound 320)

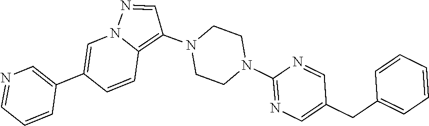 [SMILES: c1ccc(Cc2cnc(N3CCN(c4cnn5cc(-c6cccnc6)ccc45)CC3)nc2)cc1]

To a solution of 3-(piperazin-1-yl)-6-(pyridin-3-yl)pyrazolo[1,5-a]pyridine (150 mg, 0.54 mmol, as prepared in the previous step) and 5-benzyl-2-chloropyrimidine (Compound S88) (164.8 mg, 0.80 mmol) in IPA (10 mL) were added DIPEA (138.8 mg, 1.07 mmol). After stirring overnight at 80° C. under nitrogen atmosphere, the mixture was allowed to cool down to rt. The resulting mixture was concentrated under reduced pressure. The residue was dissolved in H₂O (30 mL). The resulting mixture was extracted with EtOAc (3×30 mL), dried over anhydrous Na₂SO₄, and filtered. The filtrate was concentrated under reduced pressure. The crude product was purified by Prep-HPLC to afford 46.5 mg (19%) of 3-(4-(5-benzylpyrimidin-2-yl)piperazin-1-yl)-6-(pyridin-3-yl)pyrazolo[1,5-a]pyridine (Compound 320) as a yellow solid. ¹H NMR (400 MHZ, DMSO-d₆) δ (ppm) 9.06-8.94 (m, 2H), 8.58 (dd, J=4.8, 1.5 Hz, 1H), 8.32 (s, 2H), 8.24-8.13 (m, 1H), 7.90 (s, 1H), 7.80 (d, J=9.3 Hz, 1H), 7.54-7.44 (m, 2H), 7.34-7.16 (m, 5H), 3.90 (t, J=5.0 Hz, 4H), 3.80 (s, 2H), 3.07 (t, J=5.0 Hz, 4H); MS (ESI) m/z [M+H]⁺ calcd. for C₂₇H₂₅N₇, 448.2; found, 448.1; LCMS purity: 254 nm: 98.1%.

### Example 234: Synthesis of Exemplary Compound 321

### tert-butyl 4-(6-(3-hydroxypropoxy)pyrazolo[1,5-a]pyridin-3-yl)piperazine-1-carboxylate (Compound 321)

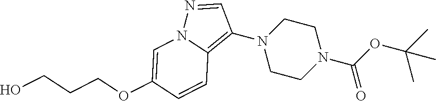 [SMILES: CC(C)(C)OC(=O)N1CCN(c2cnn3cc(OCCCO)ccc23)CC1]

### Step 1. Preparation of 6-(3-((tert-butyldimethylsilyl)oxy)propoxy)pyrazolo[1,5-a]pyridine

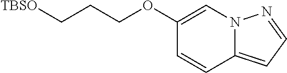 [SMILES: CC(C)(C)[Si](C)(C)OCCCOc1ccc2ccnn2c1]

To a solution of pyrazolo[1,5-a]pyridin-6-ol (0.7 g, 5.22 mmol) and K₂CO₃ (2.16 g, 15.66 mmol) in DMF (20 mL) was added tert-butyl (3-iodopropoxy) dimethylsilane (3.13 g, 10.44 mmol) at 0° C. The resulting mixture was stirred for 16 h at rt. The precipitated solids were collected by filtration and washed with DCM (3×50 mL). The combined filtrate was concentrated under reduced pressure. The residue was purified by silica gel column chromatography eluting with PE/EtOAc (3:1) to afford 1.00 g (63%) of 6-(3-((tert-butyldimethylsilyl)oxy)propoxy)pyrazolo[1,5-a]pyridine as a yellow solid. ¹H NMR (400 MHz, DMSO-d₆) δ (ppm) 8.37 (s, 1H), 7.86 (d, J=2.3 Hz, 1H), 7.61 (d, J=9.6 Hz, 1H), 7.01 (dd, J=9.6, 2.2 Hz, 1H), 6.54 (d, J=2.3 Hz, 1H), 4.07 (t, J=6.1 Hz, 2H), 3.77 (t, J=6.1 Hz, 2H), 1.92 (t, J=6.1 Hz, 2H), 0.86 (s, 9H), 0.04 (s, 6H); MS (ESI) m/z [M+H]⁺ calcd. for C₁₆H₂₆N₂O₂Si, 307.2; found, 307.2.

### Step 2. Preparation of 3-bromo-6-(3-((tert-butyldimethylsilyl)oxy)propoxy)pyrazolo[1,5-a]pyridine

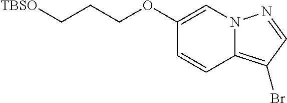 [SMILES: CC(C)(C)[Si](C)(C)OCCCOc1ccc2c(Br)cnn2c1]

To a solution of 6-(3-((tert-butyldimethylsilyl)oxy)propoxy)pyrazolo[1,5-a]pyridine (1.26 g, 4.11 mmol, as prepared in the previous step) in DCM (20.0 mL) was added NBS (0.8 g, 4.52 mmol). The resulting mixture was stirred for 1 h at rt, then concentrated under reduced pressure. The residue was purified by silica gel column chromatography eluting with to 1.34 PE/EtOAc (5:1) afford g (76%) of 3-bromo-6-(3-((tert-butyldimethylsilyl)oxy)propoxy)pyrazolo[1,5-a]pyridine as a grey solid. ¹H NMR (400 MHZ, DMSO-d₆) δ (ppm) 8.45 (d, J=1.5 Hz, 1H), 8.00 (s, 1H), 7.47 (d, J=9.6 Hz, 1H), 7.12 (dd, J=9.6, 2.1 Hz, 1H), 4.07 (t, J=6.1 Hz, 2H), 3.75 (t, J=6.1 Hz, 2H), 1.96-1.86 (m, 2H), 0.83 (s, 9H), 0.01 (s, 6H); MS (ESI) m/z [M+H]⁺ calcd. for C₁₆H₂₅BrN₂O₂Si, 385.1; found, 385.2.

### Step 3. Preparation of tert-butyl 4-(6-(3-((tert-butyldimethylsilyl)oxy)propoxy)pyrazolo[1,5-a]pyridin-3-yl)piperazine-1-carboxylate

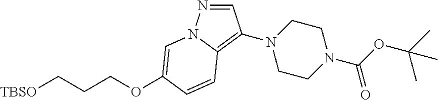 [SMILES: CC(C)(C)OC(=O)N1CCN(c2cnn3cc(OCCCO[Si](C)(C)C(C)(C)C)ccc23)CC1]

To a solution of 3-bromo-6-(3-((tert-butyldimethylsilyl)oxy)propoxy)pyrazolo[1,5-a]pyridine (500 mg, 1.30 mmol, as prepared in the previous step) and tert-butyl piperazine-1-carboxylate (1.21 g, 6.49 mmol) in tBuOH (6.0 mL) and dioxane (3.0 mL) was added KOtBu (218 mg, 1.95 mmol) and tBuXPhos Pd G1 (133 mg, 0.20 mmol) under nitrogen. The resulting mixture was stirred for 16 h at 60° C. under nitrogen. The reaction was cooled to rt, concentrated under reduced pressure, and the residue was purified by silica gel column chromatography eluting with PE/EtOAc (5:1) to afford 500 mg (71%) of tert-butyl 4-(6-(3-((tert-butyldimethylsilyl)oxy)propoxy)pyrazolo [1,5-a]pyridin-3-yl)piperazine-1-carboxylate as a yellow solid. ¹H NMR (400 MHZ, DMSO-d₆) δ (ppm) 8.21 (d, J=2.1 Hz, 1H), 7.65 (s, 1H), 7.54 (d, J=9.6 Hz, 1H), 6.87 (dd, J=9.6, 2.1 Hz, 1H), 4.03 (t, J=6.1 Hz, 2H), 3.76 (t, J=6.0 Hz, 2H), 3.48 (t, J=4.9 Hz, 4H), 2.90 (t, J=5.1 Hz, 4H), 1.96-1.85 (m, 2H), 1.42 (s, 9H), 0.85 (s, 9H), 0.03 (s, 6H); MS (ESI) m/z [M+H]⁺ calcd. for C₂₅H₄₂N₄O₄Si, 491.3; found, 491.4.

### Step 4. Preparation of tert-butyl 4-(6-(3-hydroxypropoxy)pyrazolo[1,5-a]pyridin-3-yl)piperazine-1-carboxylate (Compound 321)

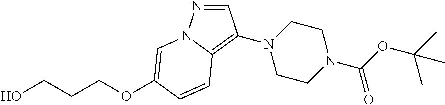 [SMILES: CC(C)(C)OC(=O)N1CCN(c2cnn3cc(OCCCO)ccc23)CC1]

To a solution of 4-(6-(3-((tert-butyldimethylsilyl)oxy)propoxy)pyrazolo[1,5-a]pyridin-3-yl)piperazine-1-carboxylate (100 mg, 0.20 mmol, as prepared in the previous step) in DMF (2.0 mL) was added CsF (31 mg, 0.20 mmol). The resulting mixture was stirred for 1 h at 80° C. The resulting mixture was filtered, and the filter cake was washed with DMF (2×0.5 mL). The filtrate was purified by Prep-HPLC to afford 45.9 mg (60%) of tert-butyl 4-(6-(3-hydroxypropoxy)pyrazolo[1,5-a]pyridin-3-yl)piperazine-1-carboxylate (Compound 321) as a light yellow solid. ¹H NMR (400 MHZ, DMSO-d₆) δ (ppm) 8.21 (d, J=2.0 Hz, 1H), 7.65 (s, 1H), 7.54 (d, J=9.6 Hz, 1H), 6.88 (dd, J=9.6, 2.1 Hz, 1H), 4.57 (t, J=5.2 Hz, 1H), 4.05 (t, J=6.3 Hz, 2H), 3.61-3.53 (m, 2H), 3.49 (t, J=4.9 Hz, 4H), 2.90 (t, J=5.0 Hz, 4H), 1.93-1.82 (m, 2H), 1.43 (s, 9H); MS (ESI) m/z [M+H]⁺ calcd. for C₁₉H₂₈N₄O₄, 377.2; found, 377.1; LCMS purity: 254 nm: 99.5%.

### Example 235: Synthesis of Exemplary Compound 322

### tert-butyl 4-(6-(3-morpholinopropoxy)pyrazolo[1,5-a]pyridin-3-yl)piperazine-1-carboxylate (Compound 322)

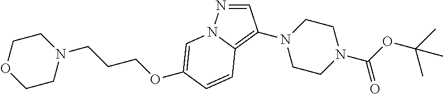 [SMILES: CC(C)(C)OC(=O)N1CCN(c2cnn3cc(OCCCN4CCOCC4)ccc23)CC1]

### Step 1. Preparation of 4-(3-(pyrazolo[1,5-a]pyridin-6-yloxy)propyl)morpholine

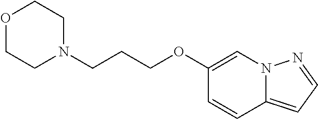 [SMILES: c1cc2ccc(OCCCN3CCOCC3)cn2n1]

To a solution of pyrazolo[1,5-a]pyridin-6-ol (500 mg, 3.73 mmol) and K₂CO₃ (1.55 g, 11.2 mmol) in DMF (10 mL) was added 4-(3-chloropropyl)morpholine (1.83 g, 11.2 mmol). The resulting mixture was stirred for 16 h at 60° C. The reaction was cooled to rt, filtered, and the filter cake was washed with DMF (2×2 mL). The combined filtrate was purified by reversed-phase flash chromatography to afford 920 mg (85%) of 4-(3-(pyrazolo[1,5-a]pyridin-6-yloxy)propyl)morpholine as a yellow solid. ¹H NMR (400 MHZ, DMSO-d₆) δ (ppm) 8.36 (d, J=2.1 Hz, 1H), 7.86 (d, J=2.3 Hz, 1H), 7.60 (d, J=9.5 Hz, 1H), 7.01 (dd, J=9.6, 2.1 Hz, 1H), 6.53 (d, J=2.3 Hz, 1H), 4.04 (t, J=6.3 Hz, 2H), 3.57 (t, J=4.6 Hz, 4H), 2.46-2.32 (m, 6H), 1.95-1.83 (m, 2H); MS (ESI) m/z [M+H]⁺ calcd. for C₁₄H₁₉N₃O₂, 262.1; found, 262.0.

### Step 2. Preparation of 4-(3-((3-bromopyrazolo[1,5-a]pyridin-6-yl)oxy)propyl)morpholine

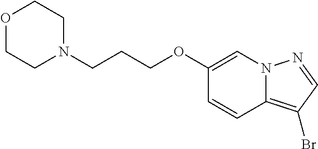 [SMILES: Brc1cnn2cc(OCCCN3CCOCC3)ccc12]

To a solution of 4-(3-(pyrazolo[1,5-a]pyridin-6-yloxy)propyl)morpholine (900 mg, 3.44 mmol, as prepared in the previous step) in DCM (10.0 mL) was added NBS (674 mg, 3.79 mmol). The resulting mixture was stirred for 1 h at rt. The resulting mixture was concentrated under reduced pressure. The residue was purified by silica gel column chromatography eluting with DCM/MeOH (12:1) to afford 900 mg (69%) of 4-(3-((3-bromopyrazolo[1,5-a]pyridin-6-yl)oxy)propyl)morpholine as a light yellow solid. ¹H NMR (400 MHZ, DMSO-d₆) δ (ppm) 8.46 (d, J=2.0 Hz, 1H), 8.02 (s, 1H), 7.49 (d, J=9.6 Hz, 1H), 7.15 (dd, J=9.6, 2.1 Hz, 1H), 4.06 (t, J=6.3 Hz, 2H), 3.57 (t, J=4.6 Hz, 4H), 2.47-2.33 (m, 6H), 1.95-1.84 (m, 2H); MS (ESI) m/z [M+H]⁺ calcd. for C₁₄H₁₈BrN₃O₂, 340.1; found, 340.0.

### Step 3. Preparation of tert-butyl 4-(6-(3-morpholinopropoxy)pyrazolo[1,5-a]pyridin-3-yl)piperazine-1-carboxylate (Compound 322)

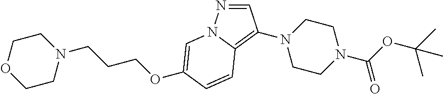 [SMILES: CC(C)(C)OC(=O)N1CCN(c2cnn3cc(OCCCN4CCOCC4)ccc23)CC1]

To a solution of 4-(3-((3-bromopyrazolo[1,5-a]pyridin-6-yl)oxy)propyl)morpholine (50 mg, 0.15 mmol, as prepared in the previous step) and tert-butyl piperazine-1-carboxylate (137 mg, 0.74 mmol) in tBuOH (2.0 mL) and dioxane (1.0 mL) was added KOtBu (24.7 mg, 0.22 mmol) and tBuXPhos Pd G1 (15.1 mg, 0.02 mmol) under nitrogen. The resulting mixture was stirred for 16 h at 60° C. under nitrogen, then cooled to rt and concentrated under reduced pressure. The residue was purified by Prep-TLC (DCM/MeOH 15:1). The crude product was purified by Prep-HPLC to afford 24.0 mg (36%) of tert-butyl 4-(6-(3-morpholinopropoxy)pyrazolo[1,5-a]pyridin-3-yl)piperazine-1-carboxylate (Compound 322) as a yellow solid. ¹H NMR (400 MHZ, DMSO-d₆) δ (ppm) 8.21 (d, J=2.1 Hz, 1H), 7.65 (s, 1H), 7.53 (d, J=9.7 Hz, 1H), 6.88 (dd, J=9.6, 2.1 Hz, 1H), 4.02 (t, J=6.3 Hz, 2H), 3.57 (t, J=4.6 Hz, 4H), 3.49 (t, J=5.0 Hz, 4H), 2.89 (t, J=5.0 Hz, 4H), 2.46-2.28 (m, 6H), 1.93-1.82 (m, 2H), 1.42 (s, 9H); MS (ESI) m/z [M+H]⁺ calcd. for C₂₃H₃₅N₅O₄, 446.3; found, 446.1; LCMS purity: 254 nm: 99.1%.

### Example 236: Synthesis of Exemplary Compound 323

### tert-butyl 4-(6-(1H-benzo[d]imidazol-2-yl)pyrazolo[1,5-a]pyridin-3-yl)piperazine-1-carboxylate (Compound 323)

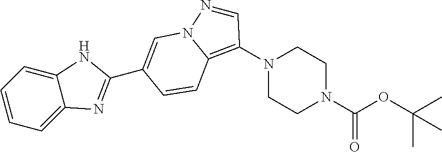 [SMILES: CC(C)(C)OC(=O)N1CCN(c2cnn3cc(-c4nc5ccccc5[nH]4)ccc23)CC1]

### Step 1. Preparation of 2-bromo-1-((2-(trimethylsilyl)ethoxy)methyl)-1H-benzo[d]imidazole

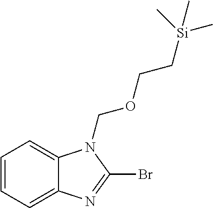 [SMILES: C[Si](C)(C)CCOCn1c(Br)nc2ccccc21]

To a solution of 2-bromo-1H-benzo[d]imidazole (1.0 g, 5.08 mmol) dissolved in DCM (50 mL) was added SEMCl (0.93 g, 5.58 mmol) and Et₃N (1.54 g, 15.23 mmol). The resulting mixture was stirred for 16 h at rt under nitrogen. The reaction was diluted with H₂O (50 mL) and extracted with DCM (3×50 mL). The combined organic layer was washed with brine (3×50 mL), dried over anhydrous Na₂SO₄, and filtered. The filtrate was concentrated under reduced pressure. The residue was purified by silica gel column chromatography eluting with PE/EtOAc (3:1) to afford 1.2 g (72%) of 2-bromo-1-((2-(trimethylsilyl)ethoxy)methyl)-1H-benzo[d]imidazole as a white solid. ¹H NMR (400 MHZ, DMSO-d₆) δ (ppm) 7.72-7.59 (m, 2H), 7.36-7.21 (m, 2H), 5.62 (s, 2H), 3.60-3.52 (m, 2H), 0.89-0.81 (m 2H), −0.10 (s, 9H); MS (ESI) m/z [M+H]⁺ calcd. for C₁₃H₁₉BrN₂OSi, 327.1; found, 327.1.

### Step 2. Preparation of 2-(pyrazolo[1,5-a]pyridin-6-yl)-1-((2-(trimethylsilyl)ethoxy)methyl)-1H-benzo[d]imidazole

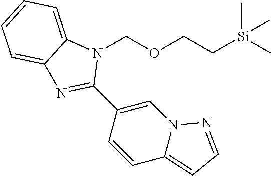 [SMILES: C[Si](C)(C)CCOCn1c(-c2ccc3ccnn3c2)nc2ccccc21]

To a solution of 2-bromo-1-((2-(trimethylsilyl)ethoxy)methyl)-1H-benzo[d]imidazole (1.0 g, 3.06 mmol, as prepared in the previous step) dissolved in dioxane (30 mL) and H₂O (3 mL) was added 6-(4,4,5,5-tetramethyl-1,3,2-dioxaborolan-2-yl)pyrazolo[1,5-a]pyridine (0.82 g, 3.36 mmol), XPhos Pd G3 (0.39 g, 0.46 mmol), XPhos (0.22 g, 0.46 mmol) and Cs₂CO₃ (1.99 g, 6.11 mmol). The resulting mixture was stirred for 6 h at rt under nitrogen. The mixture was cooled to rt then extracted with EtOAc (3×30 mL). The combined organic extracts were washed with brine (3×30 mL), dried over anhydrous Na₂SO₄, and filtered. The filtrate was concentrated under reduced pressure. The residue was purified by silica gel column chromatography, eluted with PE/EtOAc (2:1) to afford 1.0 g (89%) of 2-(pyrazolo[1,5-a]pyridin-6-yl)-1-((2-(trimethylsilyl)ethoxy)methyl)-1H-benzo[d]imidazole as a light brown solid. ¹H NMR (400 MHZ, DMSO-d₆) δ (ppm) 9.29 (s, 1H), 8.18-8.09 (m, 1H), 7.88 (d, J=9.2 Hz, 1H), 7.85-7.71 (m, 3H), 7.40-7.27 (m, 2H), 6.75 (d, J=2.2 Hz, 1H), 5.75 (s, 2H), 3.69 (t, J=8.0 Hz, 2H), 0.91 (t, J=8.0 Hz, 2H), −0.09 (s, 9H); MS (ESI) m/z [M+H]⁺ calcd. for C₂₀H₂₄N₄OSi, 365.2; found, 365.2.

### Step 3. Preparation of 2-(3-bromopyrazolo[1,5-a]pyridin-6-yl)-1-((2-(trimethylsilyl)ethoxy)methyl)-1H-benzo[d]imidazole

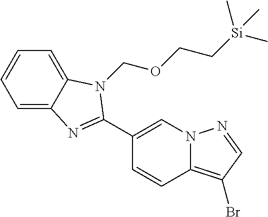 [SMILES: C[Si](C)(C)CCOCn1c(-c2ccc3c(Br)cnn3c2)nc2ccccc21]

To a solution of 2-(pyrazolo[1,5-a]pyridin-6-yl)-1-((2-(trimethylsilyl)ethoxy)methyl)-1H-benzo[d]imidazole (1.0 g, 2.74 mmol, as prepared in the previous step) dissolved in DCM (30 mL) was added NBS (0.59 g, 3.29 mmol). The resulting mixture was stirred for 2 h at rt under nitrogen. The reaction was diluted with H₂O (50 mL) and extracted with DCM (3×50 mL). The combined organic extracts were washed with brine (3×50 mL), dried over anhydrous Na₂SO₄, and filtered. The filtrate was concentrated under reduced pressure. The residue was purified by silica gel column chromatography eluting with PE/EtOAc (2:1) to afford 900 mg (73%) of 2-(3-bromopyrazolo[1,5-a]pyridin-6-yl)-1-((2-(trimethylsilyl)ethoxy)methyl)-1H-benzo[d]imidazole as a yellow solid. ¹H NMR (400 MHZ, DMSO-d₆) δ (ppm) 9.33 (t, J=1.2 Hz, 1H), 8.34 (s, 1H), 7.92 (dd, J=9.2, 1.6 Hz, 1H), 7.87-7.81 (m, 1H), 7.81-7.69 (m, 2H), 7.43-7.28 (m, 2H), 5.76 (s, 2H), 3.73-3.64 (m, 2H), 0.93-0.88 (m, 2H), −0.09 (s, 9H); MS (ESI) m/z [M+H]⁺ calcd. for C₂₀H₂₃BrN₄OSi, 443.1; found, 443.1.

### Step 4. Preparation of tert-butyl 4-(6-(1-((2-(trimethylsilyl)ethoxy)methyl)-1H-benzo[d]imidazol-2-yl)pyrazolo[1,5-a]pyridin-3-yl)piperazine-1-carboxylate

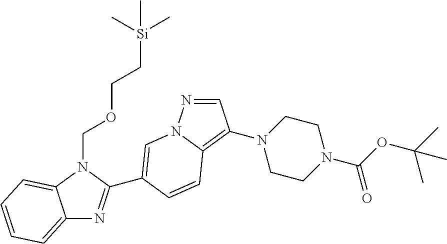 [SMILES: CC(C)(C)OC(=O)N1CCN(c2cnn3cc(-c4nc5ccccc5n4COCC[Si](C)(C)C)ccc23)CC1]

To a solution of 2-(3-bromopyrazolo[1,5-a]pyridin-6-yl)-1-((2-(trimethylsilyl)ethoxy)methyl)-1H-benzo[d]imidazole (900 mg, 2.03 mmol, as prepared in the previous step) dissolved in dioxane (10 mL) and tBuOH (10 mL) was added tert-butyl piperazine-1-carboxylate (1.89 g, 10.15 mmol), tBuXPhos Pd G1 (418.1 mg, 0.61 mmol) and KOtBu (911 mg, 8.12 mmol). The resulting solution was stirred for 16 h at 90° C. under nitrogen. The mixture was cooled to rt then diluted with H₂O (30 mL) and extracted with EtOAc (3×30 mL). The combined organic extracts were washed with brine (3×30 mL), dried over anhydrous Na₂SO₄, and filtered. The filtrate was concentrated under reduced pressure. The residue was purified by silica gel column chromatography eluting with PE/EtOAc (1:1) to afford 250 mg (22%) of 4-(6-(1-((2-(trimethylsilyl)ethoxy)methyl)-1H-benzo[d]imidazol-2-yl)pyrazolo[1,5-a]pyridin-3-yl)piperazine-1-carboxylate as a yellow solid. ¹H NMR (400 MHZ, DMSO-d₆) δ (ppm) 9.14 (s, 1H), 7.99 (s, 1H), 7.82 (dd, J=8.7, 5.3 Hz, 2H), 7.73 (d, J=7.8 Hz, 1H), 7.66-7.60 (m, 1H), 7.40-7.25 (m, 2H), 5.73 (s, 2H), 3.69 (t, J=8.0 Hz, 2H), 3.53 (t, J=5.0 Hz, 4H), 3.00 (t, J=5.0 Hz, 4H), 1.43 (s, 9H), 0.91 (t, J=8.0 Hz, 2H), −0.08 (s, 9H); MS (ESI) m/z [M+H]⁺ calcd. for C₂₉H₄₀N₆O₃Si, 549.3; found, 549.3.

### Step 5. Preparation of tert-butyl 4-(6-(1H-benzo[d]imidazol-2-yl)pyrazolo[1,5-a]pyridin-3-yl)piperazine-1-carboxylate (Compound 323)

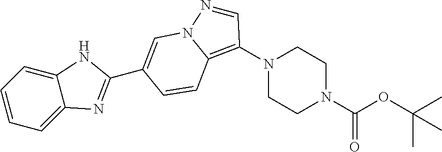 [SMILES: CC(C)(C)OC(=O)N1CCN(c2cnn3cc(-c4nc5ccccc5[nH]4)ccc23)CC1]

To a solution of tert-butyl 4-(6-(1-((2-(trimethylsilyl)ethoxy)methyl)-1H-benzo[d]imidazol-2-yl)pyrazolo[1,5-a]pyridin-3-yl)piperazine-1-carboxylate (200 mg, 0.36 mmol, as prepared in the previous step) dissolved in THF (5 mL) was added TBAF (19.1 mg, 0.073 mmol). The resulting mixture was stirred for 16 h at 80° C. under nitrogen. The mixture was cooled to rt, diluted with H₂O (30 mL), and extracted with EtOAc (6×30 mL). The combined organic extracts were washed with brine (6×30 mL), dried over anhydrous Na₂SO₄, and filtered. The filtrate was concentrated under reduced pressure. The crude product was purified by Prep-HPLC to afford 26.9 mg (18%) of tert-butyl 4-(6-(1H-benzo[d]imidazol-2-yl)pyrazolo[1,5-a]pyridin-3-yl)piperazine-1-carboxylate (Compound 323) as a yellow solid. ¹H NMR (400 MHZ, DMSO-d₆) δ (ppm) 12.97 (s, 1H), 9.30 (s, 1H), 7.97 (s, 1H), 7.88-7.79 (m, 2H), 7.67 (d, J=7.6 Hz, 1H), 7.55 (d, J=7.6 Hz, 1H), 7.22 (t, J=7.6 Hz, 2H), 3.53 (t, J=5.0 Hz, 4H), 3.00 (t, J=5.0 Hz, 4H), 1.44 (s, 9H); MS (ESI) m/z [M+H]⁺ calcd. for C₂₃H₂₆N₆O₂, 419.2; found, 419.1; LCMS purity: 254 nm: 99.7%.

### Example 237: Synthesis of Exemplary Compound 324

### tert-butyl 4-(6-(1-(2-(dimethylamino)ethyl)-1H-imidazol-4-yl)pyrazolo[1,5-a]pyridin-3-yl)piperazine-1-carboxylate (Compound 324)

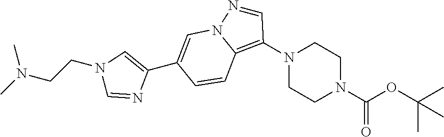 [SMILES: CN(C)CCn1cnc(-c2ccc3c(N4CCN(C(=O)OC(C)(C)C)CC4)cnn3c2)c1]

### Step 1. Preparation of 2-(4-iodo-1H-imidazol-1-yl)-N,N-dimethylethan-1-amine

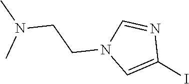

To a solution of 4-iodo-1H-imidazole (10 g, 51.55 mmol) and 2-bromo-N,N-dimethylethan-1-amine hydrobromide (14.41 g, 61.86 mmol) in ACN (100 mL) was added Cs₂CO₃ (33.6 g, 103.17 mmol). After stirring for 16 h at 80° C. under nitrogen, the reaction was cooled to rt and filtered, then filter cake was washed with ACN (3×50 mL). The combined filtrate was concentrated under reduced pressure. The residue was purified by reversed-phase chromatography to afford 800 mg (6%) of 2-(4-iodo-1H-imidazol-1-yl)-N,N-dimethylethan-1-amine as a yellow solid. ¹H NMR (400 MHZ, DMSO-d₆) δ (ppm) 7.60 (s, 1H), 7.37 (s, 1H), 4.02 (t, J=6.2 Hz, 2H), 2.55-2.50 (m, 2H), 2.14 (s, 6H); MS (ESI) m/z [M+H]⁺ calcd. for C₇H₁₂IN₃, 266.0; found, 266.1.

### Step 2. Preparation of N,N-dimethyl-2-(4-(pyrazolo[1,5-a]pyridin-6-yl)-1H-imidazol-1-yl)ethan-1-amine

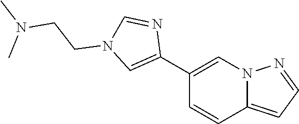 [SMILES: CN(C)CCn1cnc(-c2ccc3ccnn3c2)c1]

To a solution of 2-(4-iodo-1H-imidazol-1-yl)-N,N-dimethylethan-1-amine (800 mg, 3.02 mmol, as prepared in the previous step) and 6-(4,4,5,5-tetramethyl-1,3,2-dioxaborolan-2-yl)pyrazolo[1,5-a]pyridine (884 mg, 3.62 mmol) in dioxane (20 mL) and H₂O (0.2 mL) were added Pd(dppf)Cl₂ (441.6 mg, 0.60 mmol) and K₂CO₃ (834.1 mg, 6.04 mmol). After stirring for 16 h at 80° C. under nitrogen, the reaction was concentrated under reduced pressure. The residue was purified by Prep-TLC (CH₂Cl₂/MeOH 10:1) to afford 200.0 mg (26%) of N,N-dimethyl-2-(4-(pyrazolo[1,5-a]pyridin-6-yl)-1H-imidazol-1-yl)ethan-1-amine as a yellow solid. ¹H NMR (400 MHZ, DMSO-d₆) δ (ppm) 8.90 (s, 1H), 7.95 (d, J=2.2 Hz, 1H), 7.77-7.67 (m, 3H), 7.59 (dd, J=9.2, 1.3 Hz, 1H), 6.61-6.57 (m, 1H), 4.08 (t, J=6.5 Hz, 2H), 2.61 (t, J=6.5 Hz, 2H), 2.20 (s, 6H); MS (ESI) m/z [M+H]⁺ calcd. for C₁₄H₁₇N₅, 256.1; found, 256.1.

### Step 3. Preparation of 2-(4-(3-iodopyrazolo[1,5-a]pyridin-6-yl)-1H-imidazol-1-yl)-N,N-dimethylethan-1-amine

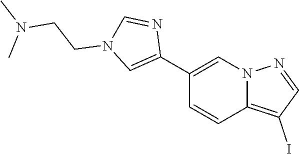 [SMILES: CN(C)CCn1cnc(-c2ccc3c(I)cnn3c2)c1]

To a solution of N,N-dimethyl-2-(4-(pyrazolo[1,5-a]pyridin-6-yl)-1H-imidazol-1-yl)ethan-1-amine (200 mg, 0.78 mmol, as prepared in the previous step) in DCM (10 mL) was added NIS (211.5 mg, 0.94 mmol). After stirring for 3 h at rt under nitrogen, the reaction was concentrated under reduced pressure. The resulting mixture was diluted with H₂O (50 mL) and extracted with DCM (3×50 mL). The combined organic extracts were washed with brine (10 mL), dried over anhydrous Na₂SO₄, and filtered. The filtrate was concentrated under reduced pressure. The residue was purified by Prep-TLC (DCM/MeOH 10:1) to afford 160 mg (54%) of 2-(4-(3-iodopyrazolo[1,5-a]pyridin-6-yl)-1H-imidazol-1-yl)-N,N-dimethylethan-1-amine as a yellow solid. ¹H NMR (400 MHZ, DMSO-d₆) δ (ppm) 8.94 (s, 1H), 8.07 (s, 1H), 7.87-7.67 (m, 3H), 7.52 (d, J=9.2 Hz, 1H), 4.08 (t, J=6.3 Hz, 2H), 2.60 (t, J=6.3 Hz, 2H), 2.19 (s, 6H); MS (ESI) m/z [M+H]⁺ calcd. for C₁₄H₁₆IN₅, 382.0; found, 382.3.

### Step 4. tert-butyl 4-(6-(1-(2-(dimethylamino)ethyl)-1H-imidazol-4-yl)pyrazolo[1,5-a]pyridin-3-yl)piperazine-1-carboxylate (Compound 324)

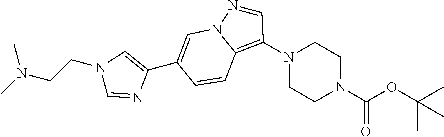 [SMILES: CN(C)CCn1cnc(-c2ccc3c(N4CCN(C(=O)OC(C)(C)C)CC4)cnn3c2)c1]

To a solution of 2-(4-(3-iodopyrazolo[1,5-a]pyridin-6-yl)-1H-imidazol-1-yl)-N,N-dimethylethan-1-amine (150 mg, 0.39 mmol, as prepared in the previous step) and tert-butyl piperazine-1-carboxylate (366.4 mg, 1.97 mmol) in dioxane (5 mL) and tBuOH (5 mL) were added tBuXPhos Pd G1 (54.5 mg, 0.08 mmol) and KOtBu (88.1 mg, 0.79 mmol). After stirring for 16 h at 90° C. under nitrogen, the reaction was concentrated under reduced pressure. The resulting mixture was diluted with H₂O (50 mL) and extracted with DCM (3×50 mL). The combined organic extracts were washed with brine (10 mL), dried over anhydrous Na₂SO₄, and filtered. The filtrate was concentrated under reduced pressure. The residue was purified by column chromatography eluting with DCM/MeOH (10:1) then the crude product was purified by Prep-HPLC to afford 25.3 mg (12%) of tert-butyl 4-(6-(1-(2-(dimethylamino)ethyl)-1H-imidazol-4-yl)pyrazolo[1,5-a]pyridin-3-yl)piperazine-1-carboxylate (Compound 324) as a light yellow solid. ¹H NMR (400 MHZ, DMSO-d₆) δ (ppm) 8.75 (s, 1H), 7.79-7.70 (m, 3H), 7.64 (dd, J=9.3, 0.9 Hz, 1H), 7.45 (dd, J=9.3, 1.5 Hz, 1H), 4.07 (t, J=6.3 Hz, 2H), 3.51 (s, 4H), 2.94 (t, J=5.0 Hz, 4H), 2.61 (d, J=6.3 Hz, 2H), 2.19 (s, 6H), 1.43 (s, 9H); MS (ESI) m/z [M+H]⁺ calcd. for C₂₃H₃₃N₇O₂, 440.3; found, 440.1; LCMS purity: 254 nm: 99.2%.

### Example 238: Synthesis of Exemplary Compound 325

### tert-butyl 4-(6-(1H-benzo[d]imidazol-6-yl)pyrazolo[1,5-a]pyridin-3-yl)piperazine-1-carboxylate (Compound 325)

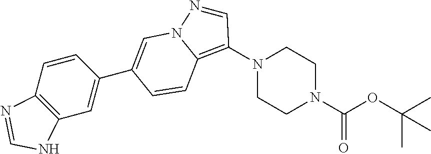 [SMILES: CC(C)(C)OC(=O)N1CCN(c2cnn3cc(-c4ccc5nc[nH]c5c4)ccc23)CC1]

### Step 1. Preparation of the mixture of 6-bromo-1-((2-(trimethylsilyl)ethoxy)methyl)-1H-benzo[d]imidazole and 5-bromo-1-((2-(trimethylsilyl)ethoxy)methyl)-1H-benzo[d]imidazole

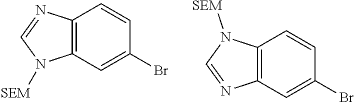 [SMILES: C[Si](C)(C)CCOCn1cnc2ccc(Br)cc21 ; C[Si](C)(C)CCOCn1cnc2cc(Br)ccc21]

To a solution of 5-bromo-3H-1,3-benzodiazole (2 g, 10.15 mmol) and TEA (2.05 g, 20.30 mmol) in DCM (30 mL) was added SEM-Cl (2.54 g, 15.22 mmol). After stirring overnight at rt under nitrogen, the reaction was washed with H₂O (100 mL), then the aqueous layer was extracted with DCM (3×100 mL). The combined organic extracts were dried over anhydrous Na₂SO₄ and filtered. The filtrate was concentrated under reduced pressure then the residue was purified by silica gel column chromatography eluting with PE/EtOAc (4:1) to afford 2 g (60%) of the mixture of 6-bromo-1-((2-(trimethylsilyl)ethoxy)methyl)-1H-benzo[d]imidazole and 5-bromo-1-((2-(trimethylsilyl)ethoxy)methyl)-1H-benzo[d]imidazole as a yellow oil. MS (ESI) m/z [M+H]⁺ calcd. for C₁₃H19BrN2OSi, 327.0; found, 327.0.

### Step 2. Preparation of the mixture of 6-(pyrazolo[1,5-a]pyridin-6-yl)-1-((2-(trimethylsilyl)ethoxy)methyl)-1H-benzo[d]imidazole and 5-(pyrazolo[1,5-a]pyridin-6-yl)-1-((2-(trimethylsilyl)ethoxy)methyl)-1H-benzo[d]imidazole

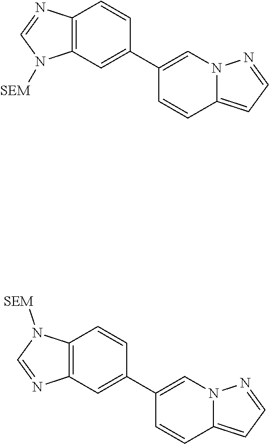 [SMILES: C[Si](C)(C)CCOCn1cnc2ccc(-c3ccc4ccnn4c3)cc21 ; C[Si](C)(C)CCOCn1cnc2cc(-c3ccc4ccnn4c3)ccc21]

To a solution of the mixture of 6-bromo-1-((2-(trimethylsilyl)ethoxy)methyl)-1H-benzo[d]imidazole and 5-bromo-1-((2-(trimethylsilyl)ethoxy)methyl)-1H-benzo[d]imidazole (2 g, 6.11 mmol, as prepared in the previous step) and 6-(4,4,5,5-tetramethyl-1,3,2-dioxaborolan-2-yl)pyrazolo[1,5-a]pyridine (2.24 g, 9.16 mmol) in dioxane (20 mL) and H₂O (4 mL) were added K₃PO₄ (2.59 g, 12.22 mmol) and Pd(dppf)Cl₂-DCM (497.79 mg, 0.61 mmol). After stirring overnight at 90° C. under nitrogen, the mixture was cooled to rt and concentrated under reduced pressure. The residue was dissolved in H₂O (200 mL), then mixture was extracted with DCM (3×200 mL). The combined organic extracts were dried over anhydrous Na₂SO₄ and filtered. The filtrate was concentrated under reduced pressure. The residue was purified by silica gel column chromatography eluting with PE/EtOAc (1:1) to afford 1.5 g (67%) of the mixture of 6-(pyrazolo[1,5-a]pyridin-6-yl)-1-((2-(trimethylsilyl)ethoxy)methyl)-1H-benzo[d]imidazole and 5-(pyrazolo[1,5-a]pyridin-6-yl)-1-((2-(trimethylsilyl)ethoxy)methyl)-1H-benzo[d]imidazole as a yellow solid. MS (ESI) m/z [M+H]⁺ calcd. for C₂₀H₂₄N₄OSi, 365.1; found, 365.1.

### Step 3. Preparation of the mixture of 6-(3-bromopyrazolo[1,5-a]pyridin-6-yl)-1-((2-(trimethylsilyl)ethoxy)methyl)-1H-benzo[d]imidazole and 5-(3-bromopyrazolo[1,5-a]pyridin-6-yl)-1-((2-(trimethylsilyl)ethoxy)methyl)-1H-benzo[d]imidazole

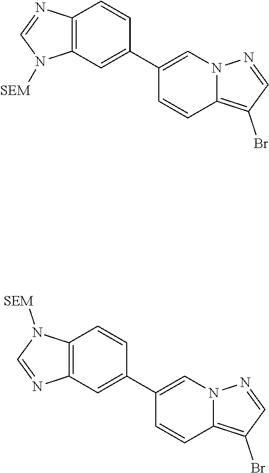 [SMILES: C[Si](C)(C)CCOCn1cnc2ccc(-c3ccc4c(Br)cnn4c3)cc21 ; C[Si](C)(C)CCOCn1cnc2cc(-c3ccc4c(Br)cnn4c3)ccc21]

To a solution of the mixture of 6-(pyrazolo[1,5-a]pyridin-6-yl)-1-((2-(trimethylsilyl)ethoxy)methyl)-1H-benzo[d]imidazole and 5-(pyrazolo[1,5-a]pyridin-6-yl)-1-((2-(trimethylsilyl)ethoxy)methyl)-1H-benzo[d]imidazole (1.1 g, 3.01 mmol, as prepared in the previous step) in DCM (30 mL) was added NBS (537.09 mg, 3.01 mmol). After stirring for 1 h at rt under nitrogen, the mixture was concentrated under reduced pressure. The residue was treated with H₂O (100 mL) and extracted with DCM (3×100 mL). The combined organic layers were dried over anhydrous Na₂SO₄ and filtered. The filtrate was concentrated under reduced pressure. The residue was purified by silica gel column chromatography eluting with PE/EtOAc (1:1) to afford 1.2 g (90%) of the mixture of 6-(3-bromopyrazolo[1,5-a]pyridin-6-yl)-1-((2-(trimethylsilyl)ethoxy)methyl)-1H-benzo[d]imidazole and 5-(3-bromopyrazolo[1,5-a]pyridin-6-yl)-1-((2-(trimethylsilyl)ethoxy)methyl)-1H-benzo[d]imidazole as a yellow solid. MS (ESI) m/z [M+H]⁺ calcd. for C₂₀H₂₃BrN₄OSi, 443.0; found, 443.0.

### Step 4. Preparation of the mixture of tert-butyl 4-(6-(1-((2-(trimethylsilyl)ethoxy)methyl)-1H-benzo[d]imidazol-6-yl)pyrazolo[1,5-a]pyridin-3-yl)piperazine-1-carboxylate and tert-butyl 4-(6-(1-((2-(trimethylsilyl)ethoxy)methyl)-1H-benzo[d]imidazol-5-yl)pyrazolo[1,5-a]pyridin-3-yl)piperazine-1-carboxylate

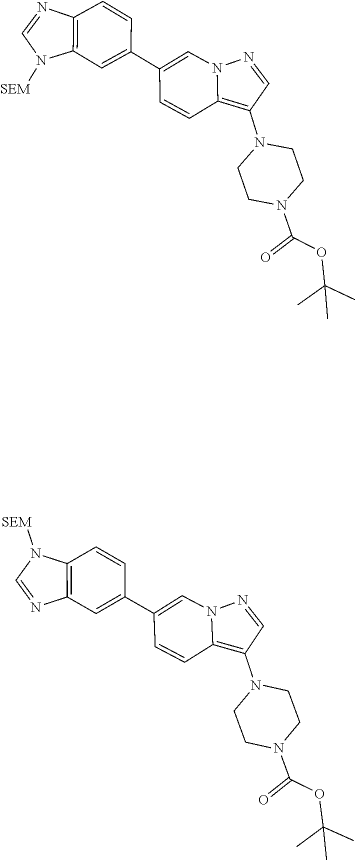 [SMILES: CC(C)(C)OC(=O)N1CCN(c2cnn3cc(-c4ccc5ncn(COCC[Si](C)(C)C)c5c4)ccc23)CC1 ; CC(C)(C)OC(=O)N1CCN(c2cnn3cc(-c4ccc5c(c4)ncn5COCC[Si](C)(C)C)ccc23)CC1]

To a solution of 6-(3-bromopyrazolo[1,5-a]pyridin-6-yl)-1-((2-(trimethylsilyl)ethoxy)methyl)-1H-benzo[d]imidazole and 5-(3-bromopyrazolo[1,5-a]pyridin-6-yl)-1-((2-(trimethylsilyl)ethoxy)methyl)-1H-benzo[d]imidazole (700 mg, 1.57 mmol, as prepared in the previous step) and tert-butyl piperazine-1-carboxylate (1.47 g, 7.89 mmol) in tBuOH (10 mL) and dioxane (10 mL) were added KOtBu (531.43 mg, 4.73 mmol) and tBuXPhos Pd G1 (325.21 mg, 0.47 mmol). After stirring for 2 days at 90° C. under nitrogen the mixture was cooled to rt and concentrated under reduced pressure. The residue was treated with H₂O (100 mL) and extracted with DCM (3×100 mL). The combined organic layers were dried over anhydrous Na₂SO₄ and filtered. The filtrate was concentrated under reduced pressure then the residue was purified by Prep-HPLC to afford 150 mg (17%) of the mixture of tert-butyl 4-(6-(1-((2-(trimethylsilyl)ethoxy)methyl)-1H-benzo[d]imidazol-6-yl)pyrazolo[1,5-a]pyridin-3-yl)piperazine-1-carboxylate and tert-butyl 4-(6-(1-((2-(trimethylsilyl)ethoxy)methyl)-1H-benzo[d]imidazol-5-yl)pyrazolo[1,5-a]pyridin-3-yl)piperazine-1-carboxylate as a yellow oil. MS (ESI) m/z [M+H]⁺ calcd. for C₂₉H₄₀N₆O₃Si, 549.2; found, 549.2.

### Step 5. tert-butyl 4-(6-(1H-benzo[d]imidazol-6-yl)pyrazolo[1,5-a]pyridin-3-yl)piperazine-1-carboxylate (Compound 325)

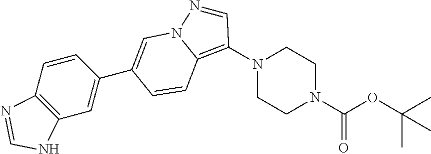 [SMILES: CC(C)(C)OC(=O)N1CCN(c2cnn3cc(-c4ccc5nc[nH]c5c4)ccc23)CC1]

To a solution of the mixture of tert-butyl 4-(6-(1-((2-(trimethylsilyl)ethoxy)methyl)-1H-benzo[d]imidazol-6-yl)pyrazolo[1,5-a]pyridin-3-yl)piperazine-1-carboxylate and tert-butyl 4-(6-(1-((2-(trimethylsilyl)ethoxy)methyl)-1H-benzo[d]imidazol-5-yl)pyrazolo[1,5-a]pyridin-3-yl)piperazine-1-carboxylate (150 mg, 0.27 mmol, as prepared in the previous step) in THF (10 mL) was added TBAF (21.44 mg, 0.08 mmol). After stirring overnight at 80° C. under nitrogen, the mixture was cooled to rt and concentrated under reduced pressure. The residue was treated with H₂O (50 mL) and extracted with EtOAc (3×50 mL). The combined organic layers were dried over anhydrous Na₂SO₄ and filtered. The filtrate was concentrated under reduced pressure. The residue was purified by Prep-HPLC to afford 50.6 mg (44%) of tert-butyl 4-(6-(1H-benzo[d]imidazol-6-yl)pyrazolo[1,5-a]pyridin-3-yl)piperazine-1-carboxylate (Compound 325) as a yellow solid. ¹H NMR (400 MHZ, DMSO-d₆) δ (ppm) 12.56 (d, J=15.8 Hz, 1H), 8.94-8.78 (m, 1H), 8.27 (d, J=3.9 Hz, 1H), 8.04-7.83 (m, 2H), 7.79-7.43 (m, 4H), 3.53 (t, J=4.9 Hz, 4H), 2.98 (t, J=4.9 Hz, 4H), 1.44 (s, 9H); MS (ESI) m/z [M+H]⁺ calcd. for C₂₃H₂₆N₆O₂, 419.1; found, 419.1; HPLC purity: 254 nm: 99.5%.

### Example 239: Functional Analysis of the Exemplary PDGFRα Inhibitors

To assess the inhibitory capability of the PDGFRα inhibitors synthesized in Examples 4-85, an enzymatic PDGFRα kinase assay was used as described below.

### Methods:

The PDGFRα enzyme assays were conducted per the manufacturer's directions (Promega PDGFRα Kinase Enzyme System and ADP Glo Assay Cat #V8011). Briefly, exemplary PDGFRα inhibitor compounds described herein were assayed in an 11-point dose response curve with a maximum concentration of 10,000 nM and 3-fold dilutions to a minimum concentration of 0.169 nM. 20 ng of purified PDGFRα protein was added to the test article. Subsequently, 150 uM of ATP and 1 μg of substrate, Poly (Glu4Tyr1) was added to each reaction, and the PDGFRα kinase mediated conversion of ATP (adenosine triphosphate) to ADP (adenosine diphosphate) was allowed to continue for two hours. ADP-Glo was added to halt the kinase reaction and deplete the remaining ATP. Kinase detection reagent, containing luciferase and luciferin, was used to convert the ADP signal into luminescence. Luminescence was measured using a Molecular Devices Spectramax ID5 and compared to a standard curve to determine kinase activity. IC₅₀s were calculated using Prism 9, Graphpad software.

### Results:

As shown in Table 3 and 4 (below), the different compounds exhibited different potencies with respect to inhibition of PDGFRα kinase activity. Of those tested, chemical compounds, e.g., 3, 6, 11, 13, 16, and 78 inhibited PDGFRα kinase activity with an IC₅₀ of less than 1,000 nM. The results confirm the inhibitory activity of the novel PDGFRα inhibitors described herein.

TABLE 3
IC50 Values on PDGFRα Kinase Inhibition
of Commercially Available Compounds

| Compound ID | IC50 (nM) |
|---|---|
| Anlotinib HCl | 1363 |
| Avapritinib | 10.8 |
| Axitinib | 976.7 |
| Bemcentinib | 428.2 |
| Cediranib | 1055.8 |
| CP-673451 | 115.4 |
| Dovitinib | 175.17 |
| ENMD-2076 | 1406.2 |
| Foretinib | 113.5 |
| JNJ-10198409 | >10,000 |
| JNJ-28312141 | 187.9 |
| K252a | 427.1 |
| Linifanib (ABT-869) | 1206 |
| Masitinib | 45.05 |
| Motesanib (AMG706) | 1163 |
| Nintedanib | 1300 |
| ON123300 | 73.48 |
| Pexidartinib (PLX3397) | >10,000 |
| R81 | 917 |
| RO4396686 | 370.35 |
| Seralutinib | 227.5 |
| TAK-593 | 845.8 |
| Tamatinib (R-406) | 359.9 |
| Tandutinib | 122.32 |
| Telatinib | 68.79 |
| Pazopanib | 1584 |
| MK 2461 | 10.82 |
| Imatinib | 12.51 |
| Sorafenib | 5.39 |

TABLE 4
IC50 Values on PDGFRα Kinase
Inhibition of Exemplary Compounds

| Compound | IC50 (nM) |
|---|---|
| Compound 6 | 406.56 |
| Compound 22 | >10,000 |
| Compound 7 | 2417 |
| Compound 8 | 1680.5 |
| Compound 23 | 423.55 |
| Compound 35 | 2089.15 |
| Compound 10 | 6193 |
| Compound 11 | 326.1 |
| Compound 26 | BLQ |
| Compound 13 | 421.25 |
| Compound 30 | 998.5 |
| Compound 15 | 1405.5 |
| Compound 3 | 883.2 |
| Compound 18 | 1646.5 |
| Compound 1 | 4007 |
| Compound 29 | 925.25 |
| Compound 16 | 282.7 |
| Compound 37 | 673.1 |
| Compound 34 | 785.5 |
| Compound 32 | 320.3 |
| Compound 36 | 684.9 |
| Compound 38 | 4754.5 |
| Compound 33 | 7.17 |
| Compound 31 | 244.25 |
| Compound 44 | 638.5 |
| Compound 52 | 1950 |
| Compound 42 | 8245.5 |
| Compound 49 | 59.49 |
| Compound 43 | 385.45 |
| Compound 51 | 422.6 |
| Compound 4 | >10,000 |
| Compound 12 | >10,000 |
| Compound 78 | <0.17* |
| Compound 2 | 1462 |
| Compound 9 | >10,000 |
| Compound 14 | 4314.5 |
| Compound 17 | 1604 |
| Compound 19 | >10,000 |
| Compound 20 | >10,000 |
| Compound 24 | >10,000 |
| Compound 25 | >10,000 |
| Compound 27 | >10,000 |
| Compound 28 | >10,000 |
| Compound 39 | >10,000 |
| Compound 40 | 2417.5 |
| Compound 45 | 1394.5 |
| Compound 46 | 121.965 |
| Compound 47 | 3989 |
| Compound 48 | >10,000 |
| Compound 50 | 3139 |
| Compound 51 | 422.6 |
| Compound 52 | 1950 |
| Compound 53 | 248.8 |
| Compound 54 | 630.4 |
| Compound 55 | 119.7 |
| Compound 56 | 1443 |
| Compound 57 | 5582 |
| Compound 58 | 300.5 |
| Compound 59 | 168.2 |
| Compound 60 | 5030 |
| Compound 61 | 5.26 |
| Compound 62 | 415.15 |
| Compound 63 | 1097 |
| Compound 64 | 38.35 |
| Compound 65 | 669.6 |
| Compound 66 | 692.9 |
| Compound 67 | 784.2 |
| Compound 68 | 1243.5 |
| Compound 69 | 153.85 |
| Compound 70 | 6695 |
| Compound 71 | 5.27 |
| Compound 72 | 2965.5 |
| Compound 73 | 116.235 |
| Compound 74 | 563 |
| Compound 75 | 6593.5 |
| Compound 76 | 923.5 |
| Compound 77 | BLQ |
| Compound 79 | 16.07 |
| Compound 80 | 328.5 |
| Compound 81 | 97.23 |
| Compound 82 | 2509.5 |
| Compound 83 | 947.5 |
| Compound 84 | 1445 |
| Compound 85 | 1570.25 |
| Compound 86 | 6369.5 |
| Compound 87 | 5935.5 |
| Compound 88 | 1603.5 |
| Compound 89 | >10,000 |
| Compound 90 | >10,000 |
| Compound 91 | >10,000 |
| Compound 92 | >10,000 |
| Compound 93 | >10,000 |
| Compound 94 | >10,000 |
| Compound 95 | >10,000 |
| Compound 96 | >10,000 |
| Compound 97 | >10,000 |
| Compound 98 | >10,000 |
| Compound 99 | >10,000 |
| Compound 100 | >10,000 |
| Compound 101 | 3418 |
| Compound 102 | 6975 |
| Compound 103 | >10,000 |
| Compound 104 | 2796 |
| Compound 105 | >10,000 |
| Compound 106 | >10,000 |
| Compound 107 | >10,000 |
| Compound 108 | >10,000 |
| Compound 109 | >10,000 |
| Compound 110 | >10,000 |
| Compound 111 | >10,000 |
| Compound 112 | >10,000 |
| Compound 113 | >10,000 |
| Compound 114 | >10,000 |
| Compound 115 | >10,000 |
| Compound 116 | 6519.5 |
| Compound 117 | >10,000 |
| Compound 118 | >10,000 |
| Compound 119 | 1712.7 |
| Compound 120 | >10,000 |
| Compound 121 | 817.8 |
| Compound 122 | >10,000 |
| Compound 123 | >10,000 |
| Compound 124 | >10,000 |
| Compound 125 | >10,000 |
| Compound 126 | 7116 |
| Compound 127 | >10,000 |
| Compound 128 | >10,000 |
| Compound 129 | >10,000 |
| Compound 130 | >10,000 |
| Compound 131 | 3878 |
| Compound 132 | 743.8 |
| Compound 133 | 1436 |
| Compound 134 | 3004.5 |
| Compound 135 | 3634 |
| Compound 136 | >10,000 |
| Compound 137 | 5051.5 |
| Compound 138 | 9007.5 |
| Compound 139 | 8805.5 |
| Compound 140 | >10,000 |
| Compound 141 | >10,000 |
| Compound 142 | 9537 |
| Compound 143 | 615.85 |
| Compound 144 | 4531.5 |
| Compound 145 | 6971 |
| Compound 146 | 5694 |
| Compound 147 | 6723 |
| Compound 148 | 8163 |
| Compound 149 | >10,000 |
| Compound 150 | 1140.75 |
| Compound 151 | 2784 |
| Compound 152 | 7187.5 |
| Compound 153 | 1368.35 |
| Compound 154 | 697.3 |
| Compound 155 | 58.584 |
| Compound 156 | 245.25 |
| Compound 157 | 2105 |
| Compound 158 | >10,000 |
| Compound 159 | 449.5 |
| Compound 160 | 9681.5 |
| Compound 161 | 2110.5 |
| Compound 162 | 1475.5 |
| Compound 163 | 349.85 |
| Compound 164 | 783.8 |
| Compound 165 | 685.65 |
| Compound 166 | 3568.5 |
| Compound 167 | 106.785 |
| Compound 168 | 104.695 |
| Compound 169 | 825 |
| Compound 170 | 2379.5 |
| Compound 171 | >10,000 |
| Compound 172 | 710.45 |
| Compound 173 | 1097.9 |
| Compound 174 | 305.65 |
| Compound 175 | 220.115 |
| Compound 176 | 2076.5 |
| Compound 177 | 75.3 |
| Compound 178 | 2073.5 |
| Compound 179 | 661.9 |
| Compound 180 | 4334.5 |
| Compound 181 | 1858 |
| Compound 182 | 6992 |
| Compound 183 | 814.9 |
| Compound 184 | 126 |
| Compound 185 | 510.04 |
| Compound 186 | >10,000 |
| Compound 187 | 153.38 |
| Compound 188 | 5100.3 |
| Compound 189 | 5279 |
| Compound 190 | 543.35 |
| Compound 191 | 286.65 |
| Compound 192 | 31.0435 |
| Compound 193 | 69.125 |
| Compound 194 | 194.75 |
| Compound 195 | 1230.5 |
| Compound 196 | 522.4 |
| Compound 197 | 65.305 |
| Compound 199 | 1243.75 |
| Compound 200 | 6957 |
| Compound 201 | >10,000 |
| Compound 203 | >10,000 |
| Compound 204 | >10,000 |
| Compound 205 | >10,000 |
| Compound 206 | 5342.5 |
| Compound 207 | 4829.5 |
| Compound 208 | >10,000 |
| Compound 209 | >10,000 |
| Compound 210 | 7476.5 |
| Compound 211 | 7376.5 |
| Compound 212 | 3665.5 |
| Compound 213 | >10,000 |
| Compound 214 | >10,000 |
| Compound 215 | >10,000 |
| Compound 216 | 1003.7 |
| Compound 217 | 593.95 |
| Compound 218 | 2306 |
| Compound 219 | >10,000 |
| Compound 220 | 528.3 |
| Compound 221 | 505.85 |
| Compound 222 | 912.3 |
| Compound 223 | 9253.5 |
| Compound 224 | >10,000 |
| Compound 225 | >10,000 |
| Compound 226 | 1423.5 |
| Compound 227 | 268.45 |
| Compound 228 | >10,000 |
| Compound 229 | 363.35 |
| Compound 230 | >10,000 |
| Compound 231 | 394.7 |
| Compound 232 | >10,000 |
| Compound 233 | 692.2 |
| Compound 234 | 311.27 |
| Compound 235 | 467.05 |
| Compound 236 | 695.65 |
| Compound 237 | >10,000 |
| Compound 238 | 429.75 |
| Compound 239 | >10,000 |
| Compound 240 | 9406 |
| Compound 241 | 974.1 |
| Compound 242 | >10,000 |
| Compound 243 | >10,000 |
| Compound 244 | 2137 |
| Compound 245 | >10,000 |
| Compound 246 | 936.85 |
| Compound 247 | 1780 |
| Compound 248 | 1193 |
| Compound 249 | 1256 |
| Compound 250 | 80.44 |
| Compound 251 | 1157.35 |
| Compound 252 | 673.95 |
| Compound 253 | >10,000 |
| Compound 254 | 267 |
| Compound 255 | 1142 |
| Compound 256 | 3198.15 |
| Compound 257 | 79.755 |
| Compound 258 | 802.55 |
| Compound 259 | >10,000 |
| Compound 260 | 376.8 |
| Compound 261 | 17.095 |
| Compound 262 | >10,000 |
| Compound 263 | 516.8 |
| Compound 264 | 26.8085 |
| Compound 265 | BLQ |
| Compound 266 | 2.69 |
| Compound 267 | BLQ |
| Compound 268 | 11.81545 |
| Compound 269 | 137.825 |
| Compound 270 | 157.75 |
| Compound 271 | 1488 |
| Compound 272 | 865.3 |
| Compound 273 | 12.97 |
| Compound 274 | 49.011 |
| Compound 275 | 67.015 |
| Compound 276 | 2704 |
| Compound 277 | 207.98 |
| Compound 278 | 1399.5 |
| Compound 279 | 58.855 |
| Compound 280 | 6177 |
| Compound 281 | 302.515 |
| Compound 282 | BLQ |
| Compound 283 | BLQ |
| Compound 284 | >10,000 |
| Compound 285 | 7620 |
| Compound 286 | 1792.8 |
| Compound 287 | 2466 |
| Compound 288 | BLQ |
| Compound 289 | 430 |
| Compound 290 | 0.56 |
| Compound 291 | 0.016 |
| Compound 292 | 49.995 |
| Compound 293 | 5245.3 |
| Compound 294 | 5.965 |
| Compound 295 | 31.705 |
| Compound 296 | 396.6 |
| Compound 298 | >10,000 |
| Compound 299 | >10,000 |
| Compound 300 | 3552 |
| Compound 301 | >10,000 |
| Compound 302 | 137.39 |
| Compound 303 | >10,000 |
| Compound 304 | >10,000 |
| Compound 306 | 2.315 |
| Compound 307 | 2150 |
| Compound 308 | 2108 |
| Compound 309 | 823.9 |
| Compound 310 | >10,000 |
| Compound 311 | 39.09 |
| Compound 312 | 144.6 |
| Compound 313 | BLQ |
| Compound 314 | 7552.5 |
| Compound 315 | 547.2 |
| Compound 316 | 194.4 |
| Compound 317 | 17.305 |

*Below limit of quantitation (BLQ).

### Example 240: Analysis of the Effect of PDGFRα Inhibition on GPR17 Expression In Vitro

To assess the effects of the PDGFRα inhibitors on OPC differentiation, primary OPCs were cultured and exposed to compounds. The number of newly generated oligodendrocytes was assessed by monitoring GPR17 expression. GPR17 is preferentially expressed in actively differentiating oligodendrocytes, allowing them to be distinguished from precursors and more differentiated oligodendrocytes.

### Methods:

### OPC Isolation and Seeding:

Cortices from postnatal day (P) 1-3 rats were dissected and collected into ice cold Hank's Balanced Salt Solution (HBSS). A homogenous cell suspension of the cortices was prepared using the Neural Tissue Dissociation Kit (P) (Miltenyi #130-092-628) as per manufacturer's protocol. Oligodendrocyte progenitor cells (OPCs) were isolated from the mixed cortical cell suspension by positive selection using Anti-A2B5 MicroBeads (Miltenyi #130-093-388), as per manufacturer's protocol. Briefly, the cortical cell suspension was centrifuged and washed with buffer (0.5% BSA in PBS) and labeled with Anti-A2B⁵ MicroBeads (10 μL beads per 1×10⁷ cells) for 15 minutes at 4° C. Following the incubation, cells were washed once with BSA buffer to remove unlabeled beads. A2B⁵ positive cells were eluted on the column using the AutoMACS® (Miltenyi, as per manufacturer's protocol using “Possel-s” program). Eluted cells were further washed twice with OPC growth media. Cell counts were performed using Countess III cell counter (ThermoFisher; AMQAX2000) and cells were plated.

Following cell count, OPCs were seeded at a density of approximately 10,000 cells per well in poly-D-lysine coated 96 well plates (PE Cell Carrier #6005550) or approximately 2500 cells per well in poly-D-lysine coated 384 well plate (PE Cell Carrier #6057500) in 90 μL of OPC growth medium (DMEM/F12+B27 without vitamin A+pen/strep) supplemented with 10 ng/ml of PDGF-AA (Peprotech 100-13A) and 10 ng/ml of FGF (fibroblast growth factor; R&D Systems #233 FB 010). The plates were allowed to sit at room temperature for 15 minutes to ensure proper dispersion of cells in a well. The plates were then transferred to the incubator (37° C.; 5% CO₂).

### Compound Treatment

Following 3-4 hours of incubation (to allow cells to adhere), cells (seeded in 90 ul OPC growth media) were treated with 10 ul of 10× concentration of vehicle (DMSO; 0.05% or 0.03%) control (Santa Cruz Biotechnology; sc-358801), commercially available PDGFRαinhibitors (5 μM or 3 μM), or newly constructed PDGFRα inhibitors (5 μM or 3 μM), resulting in a final 1× concentration of 0.005% or 0.003% for the vehicle or 0.5 μM or 0.3 μM for the PDGFRα inhibitors. Cells were then incubated for 72 hours at 37° C., 5% CO₂ before immunostaining for GPR17 marker expression.

### Immunocytochemistry:

Following 72 hours in culture, cells were fixed with 4% paraformaldehyde for 15 minutes and then washed three times in Dulbecco's phosphate buffered saline (DPBS). Prior to immunostaining, cells were treated with a blocking solution (10% normal goat serum containing 1% BSA and 0.1% Triton® X-100 in DPBS) for 15 minutes at room temperature. Primary antibody (anti-GPR17 at 1:2000; Origene TA811615) was applied to cells overnight at 4° C. in incubation solution containing 1% normal goat serum, 1% BSA, and 0.1% Triton X-100 in DPBS. Following overnight primary incubation, cells were washed three times in DPBS, and then, secondary antibody (Goat anti-mouse Alexa Fluor 594 at 1:1000, Jackson Immunoresearch 112-545-003) was applied to the cells. The secondary antibody was applied in incubation solution for 2 hours at room temperature, protected from light. Following incubation with the secondary antibodies, cells were washed three times with DPBS and nuclei stained with Hoechst (BioRad #1351304) before imaging.

### Image Analysis and Quantification:

Cells were imaged using ImageXpress® confocal system (Molecular Devices; 5150105) and analyzed using a custom protocol developed using MetaXpress Custom Module software. The number of differentiated GPR17+ cells was normalized to the total nuclei count and percent differentiated GPR17+ cells were reported. Statistical analysis was done using one-way ANOVA with multiple comparisons and post-hoc Tukey test in GraphPad Prism (GraphPad Software).

### Results:

Treatment of the OPCs with PDGFRα inhibitors led to an increase in differentiated GPR17+ cells, a marker of oligodendrocyte differentiation. The increase is represented by fold change over vehicle for the commercially available PDGFRα inhibitors (Table 5) and newly constructed PDGFRα inhibitors (Table 6). These results demonstrate that PDGFRα inhibition induces OPC differentiation, indicating that compounds that inhibit PDGFRα activity could be useful for inducing remyelination.

TABLE 5
In vitro GPR17 Expression Induced by Commercially
Available PDGFRα Inhibitors

| | GPR17 Fold Induction |
|---|---|
| Compound ID | (over vehicle; 5 μM) |
| Anlotinib HCl | 6.58 |
| Avapritinib | 3.97 |
| Axitinib | 5.99 |
| Bemcentinib | 2.99 |
| Cediranib | 7.47 |
| CP-673451 | 2.94 |
| Dovitinib | 6.03 |
| ENMD-2076 | 4.59 |
| Foretinib | 6.97 |
| JNJ-10198409 | 3.87 |
| JNJ-28312141 | 3.06 |
| K252a | 2.53 |
| Linifanib (ABT-869) | 4.68 |
| Masitinib | 3.50 |
| Motesanib (AMG706) | 3.00 |
| Nintedanib | 5.99 |
| ON123300 | 6.72 |
| Pexidartinib (PLX3397) | 3.55 |
| R81 | 5.91 |
| RO4396686 | 3.61 |
| Seralutinib | 3.53 |
| SU-14813 | 4.12 |
| TAK-593 | 5.82 |
| Tamatinib (R-406) | 5.55 |
| Tandutinib | 4.57 |
| Telatinib | 3.90 |
| Pazopanib | 8.70 |
| MK 2461 | 7.10 |
| Imatinib | 4.30 |
| Sorafenib | 5.30 |

TABLE 6
In vitro GPR17 Expression Induced
by Exemplary PDGFRα Inhibitors

| | GPR17 Fold Induction | GPR17 Fold Induction |
|---|---|---|
| Compound | (Over Vehicle; 5 μM) | (Over Vehicle; 3 μM) |
| Compound 6 | 4.07 | 4.47 |
| Compound 22 | 3.46 | 6.13 |
| Compound 7 | 4.40, 4.45 | 7.38 |
| Compound 8 | 3.18 | 10.86 |
| Compound 23 | 3.98 | 9.53 |
| Compound 35 | 3.57 | 7.17 |
| Compound 10 | 3.81 | 5.65 |
| Compound 11 | 4.22 | 10.21 |
| Compound 26 | 4.97 | 13.87 |
| Compound 13 | 4.31 | 8.42 |
| Compound 30 | 3.25 | 6.05 |
| Compound 15 | 3.98 | 6.37 |
| Compound 3 | 3.69 | 4.12 |
| Compound 18 | 3.50 | 4.40 |
| Compound 1 | 2.94 | 5.25 |
| Compound 29 | 3.20 | 9.41 |
| Compound 16 | 2.39 | 6.82 |
| Compound 37 | 7.65 | 5.81 |
| Compound 34 | 5.12 | 5.16 |
| Compound 32 | 6.99 | 10.59 |
| Compound 36 | 5.22 | 7.08 |
| Compound 38 | 6.02 | 7.04 |
| Compound 33 | 7.14 | 13.76 |
| Compound 31 | 6.22 | 9.76 |
| Compound 44 | 3.74 | 4.28 |
| Compound 52 | 5.41 | 9.09 |
| Compound 42 | 4.64 | 10.21 |
| Compound 49 | 4.42 | 9.99 |
| Compound 43 | 6.92 | 12.48 |
| Compound 51 | 2.52 | 5.14 |
| Compound 78 | — | 13.71 |
| Compound 2 | — | 1.46 |
| Compound 9 | — | 1.88 |
| Compound 12 | — | 0.68 |
| Compound 14 | — | 1.11 |
| Compound 17 | — | 1.14 |
| Compound 19 | — | 0.83 |
| Compound 20 | — | 0.58 |
| Compound 24 | — | 0.54 |
| Compound 25 | — | 1.07 |
| Compound 27 | — | 0.96 |
| Compound 28 | — | 1.84 |
| Compound 39 | — | 1.78 |
| Compound 40 | — | 1.27 |
| Compound 45 | — | 1.39 |
| Compound 46 | — | 6.86 |
| Compound 47 | — | 1.13 |
| Compound 48 | — | 2.75 |
| Compound 50 | — | 5.00 |
| Compound 53 | — | 5.60 |
| Compound 54 | — | 6.81 |
| Compound 55 | — | 8.97 |
| Compound 56 | — | 4.54 |
| Compound 57 | — | 5.28 |
| Compound 58 | — | 7.31 |
| Compound 59 | — | 5.49 |
| Compound 60 | — | 2.92 |
| Compound 61 | — | 10.09 |
| Compound 62 | — | 6.53 |
| Compound 63 | — | 8.97 |
| Compound 64 | — | 6.09 |
| Compound 65 | — | 5.18 |
| Compound 66 | — | 5.07 |
| Compound 67 | — | 3.41 |
| Compound 68 | — | 4.35 |
| Compound 69 | — | 4.53 |
| Compound 70 | — | 3.72 |
| Compound 71 | — | 9.66 |
| Compound 72 | — | 4.75 |
| Compound 73 | — | 6.68 |
| Compound 74 | — | 7.19 |
| Compound 75 | — | 3.34 |
| Compound 76 | — | 6.18 |
| Compound 77 | — | 7.12 |
| Compound 79 | — | 5.46 |
| Compound 80 | — | 8.19 |
| Compound 81 | — | 6.74 |
| Compound 82 | — | 6.50 |
| Compound 83 | — | 6.49 |
| Compound 84 | — | 1.33 |
| Compound 85 | — | 0.82 |
| Compound 86 | — | 0.65 |
| Compound 87 | — | 1.43 |
| Compound 88 | — | 2.90 |
| Compound 89 | — | 1.53 |
| Compound 90 | — | 0.92 |
| Compound 91 | — | 0.69 |
| Compound 92 | — | 0.81 |
| Compound 93 | — | 0.88 |
| Compound 94 | — | 1.00 |
| Compound 95 | — | 1.16 |
| Compound 96 | — | 0.50 |
| Compound 97 | — | 1.25 |
| Compound 98 | — | 0.98 |
| Compound 99 | — | 0.62 |
| Compound 100 | — | 0.83 |
| Compound 101 | — | 0.67 |
| Compound 102 | — | 0.58 |
| Compound 103 | — | 0.65 |
| Compound 104 | — | 1.40 |
| Compound 105 | — | 0.46 |
| Compound 106 | — | 0.72 |
| Compound 107 | — | 0.58 |
| Compound 108 | — | 0.54 |
| Compound 109 | — | 1.38 |
| Compound 110 | — | 0.85 |
| Compound 111 | — | 0.60 |
| Compound 112 | — | 0.91 |
| Compound 113 | — | 0.57 |
| Compound 114 | — | 0.33 |
| Compound 115 | — | 2.46 |
| Compound 116 | — | 4.31 |
| Compound 117 | — | 1.39 |
| Compound 118 | — | 0.93 |
| Compound 119 | — | 8.04 |
| Compound 120 | — | 0.87 |
| Compound 121 | — | 4.71 |
| Compound 122 | — | 3.19 |
| Compound 123 | — | 1.34 |
| Compound 124 | — | 1.47 |
| Compound 125 | — | 1.09 |
| Compound 126 | — | 5.91 |
| Compound 127 | — | 1.36 |
| Compound 128 | — | 1.45 |
| Compound 129 | — | 2.27 |
| Compound 130 | — | 1.57 |
| Compound 131 | — | 6.36 |
| Compound 132 | — | 5.85 |
| Compound 133 | — | 3.02 |
| Compound 134 | — | 4.19 |
| Compound 135 | — | 7.38 |
| Compound 136 | — | 3.64 |
| Compound 137 | — | 0.99 |
| Compound 138 | — | 0.98 |
| Compound 139 | — | 3.87 |
| Compound 140 | — | 3.54 |
| Compound 141 | — | 1.09 |
| Compound 142 | — | 5.85 |
| Compound 143 | — | 3.89 |
| Compound 144 | — | 2.64 |
| Compound 145 | — | 1.73 |
| Compound 146 | — | 4.45 |
| Compound 147 | — | 2.23 |
| Compound 148 | — | 7.82 |
| Compound 149 | — | 8.88 |
| Compound 150 | — | 4.10 |
| Compound 151 | — | 20.40 |
| Compound 152 | — | 4.33 |
| Compound 153 | — | 6.37 |
| Compound 154 | — | 13.45 |
| Compound 155 | — | 5.02 |
| Compound 156 | — | 3.92 |
| Compound 157 | — | 2.80 |
| Compound 158 | — | 4.57 |
| Compound 159 | — | 3.62 |
| Compound 160 | — | 2.80 |
| Compound 161 | — | 3.97 |
| Compound 162 | — | 2.90 |
| Compound 163 | — | 5.84 |
| Compound 164 | — | 5.94 |
| Compound 165 | — | 18.42 |
| Compound 166 | — | 6.36 |
| Compound 167 | — | 7.07 |
| Compound 168 | — | 6.43 |
| Compound 169 | — | 9.79 |
| Compound 170 | — | 3.96 |
| Compound 171 | — | 5.33 |
| Compound 172 | — | 4.90 |
| Compound 173 | — | 6.75 |
| Compound 174 | — | 6.67 |
| Compound 175 | — | 6.34 |
| Compound 176 | — | 3.90 |
| Compound 177 | — | 10.12 |
| Compound 178 | — | 5.98 |
| Compound 179 | — | 5.37 |
| Compound 180 | — | 4.05 |
| Compound 181 | — | 4.87 |
| Compound 182 | — | 13.16 |
| Compound 183 | — | 5.74 |
| Compound 184 | — | 9.26 |
| Compound 185 | — | 12.67 |
| Compound 186 | — | 1.26 |
| Compound 187 | — | 4.97 |
| Compound 188 | — | 6.03 |
| Compound 189 | — | 7.61 |
| Compound 190 | — | 6.05 |
| Compound 191 | — | 23.57 |
| Compound 192 | — | 14.76 |
| Compound 193 | — | 11.72 |
| Compound 194 | — | 8.67 |
| Compound 195 | — | 5.57 |
| Compound 196 | — | 5.65 |
| Compound 197 | — | 12.24 |
| Compound 198 | — | 9.23 |
| Compound 199 | — | 4.08 |
| Compound 200 | — | 2.20 |
| Compound 201 | — | 1.30 |
| Compound 202 | — | 1.94 |
| Compound 203 | — | 1.35 |
| Compound 204 | — | 1.20 |
| Compound 205 | — | 1.92 |
| Compound 206 | — | 3.32 |
| Compound 207 | — | 2.24 |
| Compound 208 | — | 1.22 |
| Compound 209 | — | 2.02 |
| Compound 210 | — | 4.38 |
| Compound 211 | — | 2.54 |
| Compound 212 | — | 2.51 |
| Compound 213 | — | 0.68 |
| Compound 214 | — | 2.00 |
| Compound 215 | — | 0.76 |
| Compound 216 | — | 1.92 |
| Compound 217 | — | 1.69 |
| Compound 218 | — | 3.46 |
| Compound 219 | — | 1.42 |
| Compound 220 | — | 5.20 |
| Compound 221 | — | 6.61 |
| Compound 222 | — | 3.74 |
| Compound 223 | — | 1.13 |
| Compound 224 | — | 0.66 |
| Compound 225 | — | 1.55 |
| Compound 226 | — | 11.69 |
| Compound 227 | — | 9.02 |
| Compound 228 | — | 1.19 |
| Compound 229 | — | 10.61 |
| Compound 230 | — | 0.97 |
| Compound 231 | — | 9.34 |
| Compound 232 | — | 1.03 |
| Compound 233 | — | 9.27 |
| Compound 234 | — | 7.16 |
| Compound 235 | — | 9.32 |
| Compound 236 | — | 6.03 |
| Compound 237 | — | 0.95 |
| Compound 238 | — | 6.34 |
| Compound 239 | — | 1.88 |
| Compound 240 | — | 4.67 |
| Compound 241 | — | 16.27 |
| Compound 242 | — | 2.24 |
| Compound 243 | — | 0.62 |
| Compound 244 | — | 4.76 |
| Compound 245 | — | 5.22 |
| Compound 246 | — | 3.04 |
| Compound 247 | — | 2.45 |
| Compound 248 | — | 3.90 |
| Compound 249 | — | 6.40 |
| Compound 250 | — | 9.74 |
| Compound 251 | — | 4.97 |
| Compound 252 | — | 1.79 |
| Compound 253 | — | 0.97 |
| Compound 254 | — | 6.77 |
| Compound 255 | — | 8.56 |
| Compound 256 | — | 4.15 |
| Compound 257 | — | 10.85 |
| Compound 258 | — | 9.21 |
| Compound 259 | — | 2.69 |
| Compound 260 | — | 6.26 |
| Compound 261 | — | 5.04 |
| Compound 262 | — | 2.61 |
| Compound 263 | — | 5.98 |
| Compound 264 | — | 6.72 |
| Compound 265 | — | 7.72 |
| Compound 266 | — | 4.88 |
| Compound 267 | — | 5.62 |
| Compound 268 | — | 5.57 |
| Compound 269 | — | 5.70 |
| Compound 270 | — | 7.71 |
| Compound 271 | — | 6.67 |
| Compound 272 | — | 6.11 |
| Compound 273 | — | 9.05 |
| Compound 274 | — | 5.01 |
| Compound 274 | — | 9.78 |
| Compound 275 | — | 6.70 |
| Compound 276 | — | 2.11 |
| Compound 277 | — | 5.36 |
| Compound 278 | — | 6.13 |
| Compound 279 | — | 3.62 |
| Compound 280 | — | 3.05 |
| Compound 281 | — | 11.42 |
| Compound 282 | — | 6.40 |
| Compound 283 | — | 5.90 |
| Compound 284 | — | 6.21 |
| Compound 285 | — | 6.20 |
| Compound 286 | — | 4.95 |
| Compound 287 | — | 6.48 |
| Compound 288 | — | 5.92 |
| Compound 289 | — | 7.44 |
| Compound 290 | — | 10.47 |
| Compound 291 | — | 6.24 |
| Compound 292 | — | 9.78 |
| Compound 293 | — | 7.29 |
| Compound 294 | — | 9.58 |
| Compound 295 | — | 8.65 |
| Compound 296 | — | 10.65 |
| Compound 298 | — | 1.56 |
| Compound 299 | — | 1.43 |
| Compound 300 | — | 1.24 |
| Compound 301 | — | 2.96 |
| Compound 302 | — | 8.86 |
| Compound 303 | — | 2.52 |
| Compound 304 | — | 1.77 |
| Compound 305 | — | 1.15 |
| Compound 306 | — | 4.37 |
| Compound 307 | — | 4.20 |
| Compound 308 | — | 3.67 |
| Compound 309 | — | 6.67 |
| Compound 310 | — | 4.86 |
| Compound 311 | — | 5.77 |
| Compound 312 | — | 9.00 |
| Compound 313 | — | 10.56 |
| Compound 314 | — | 1.64 |
| Compound 315 | — | 4.27 |
| Compound 316 | — | 9.10 |
| Compound 317 | — | 12.64 |

### Example 241: Analysis of the Effect of PDGFRα Inhibition on MBP Expression In Vitro

While expression of GPR17 indicates differentiated or pre-myelinating oligodendrocytes, terminal differentiation of the OPCs to oligodendrocytes and formation of myelin is characterized by expression of myelin basic protein (MBP), which is an important element of myelin

To further confirm the results shown in Example 240 that PDGFRα inhibitors can lead to terminal oligodendrocyte differentiation, select PDGFRα inhibitors from Example 240 were tested in the in vitro OPC differentiation assay by measuring the number of MBP+ oligodendrocytes.

### Methods:

### OPC Isolation and Seeding:

Cortices from postnatal day (P) 1-3 rats were dissected and collected into ice cold Hank's Balanced Salt Solution (HBSS). A homogenous cell suspension of the cortices was prepared using the Neural Tissue Dissociation Kit (P) (Miltenyi #130-092-628) as per manufacturer's protocol. Oligodendrocyte progenitor cells (OPCs) were isolated from the mixed cortical cell suspension by positive selection using Anti-A2B5 MicroBeads (Miltenyi #130-093-388), as per manufacturer's protocol. Briefly, the cortical cell suspension was centrifuged and washed with buffer (0.5% BSA in PBS) and labeled with Anti-A2B5 MicroBeads (10 μL beads per 1×10⁷ cells) for 15 minutes at 4° C. Following the incubation, cells were washed once with BSA buffer to remove unlabeled beads. A2B⁵ positive cells were eluted on the column using the AutoMACS® (Miltenyi, as per manufacturer's protocol using “possel-s” program). Eluted cells were further washed twice with OPC growth media. Cell count was performed using Countess III cell counter (ThermoFisher; AMQAX2000) and cells were plated.

A2B5+ OPCs were plated in poly-d-lysine (PDL) coated T75 flask at a density of ˜3 million cells per flask. Cells were allowed to expand for 3 days at 37° C., 5% CO₂ in OPC growth media containing 10 ng/ml of PDGF (Peprotech 100-13A) and 10 ng/ml FGF (R&D Systems #233-FB-010). Following three days of expansion (˜60-70% confluency), cells were lifted using TrypLE (Thermo 12604013) for 3-5 minutes at 37° C., 5% CO₂. TrypLE was normalized by adding 10 mL growth media and cells were centrifuged at 1400 rpm for 10 minutes. Cell count was performed, and cells were plated into PDL coated 96 well plates (PE Cell Carrier #6005550) at approximately 5000 cells per well in 90 μL of OPC growth medium (DMEM/F12+B27 without vitamin A+pen/strep) supplemented with 10 ng/ml of PDGF-AA (Peprotech 100-13A) and 10 ng/ml of FGF (fibroblast growth factor; R&D Systems #233 FB 010). The plates were allowed to sit at room temperature for 15 minutes to ensure proper dispersion of cells in a well. The plates were then transferred to the incubator (37° C.; 5% CO₂).

### Compound Treatment

Following 3-4 hours of incubation (to allow cells to adhere), cells (seeded in 90 ul OPC growth media) were treated with 10 ul of 10× concentration of vehicle (DMSO; 0.03%) control (Santa Cruz Biotechnology; sc-358801), commercially available PDGFRα inhibitors (3 uM), or newly constructed PDGFRα inhibitors (3 uM), resulting in a final 1× concentration of 0.3% for the vehicle or 0.3 uM for the PDGFRα inhibitors. Cells were then incubated for 72 hours at 37° C., 5% CO₂ before immunostaining for MBP marker expression.

### Immunocytochemistry:

Following 72 hours of treatment, cells were fixed with 4% paraformaldehyde for 15 minutes then washed three times in DPBS. Prior to staining, cells were blocked for 15 minutes in 10% normal goat serum containing 1% BSA and 0.1% Triton X-100 in DPBS. Primary antibody (MBP @ 1:2000; Abcam AB7349) was applied to cells overnight at 4° C. in incubation solution containing 1% normal goat serum, 1% BSA, and 0.1% Triton X-100 in DPBS. After primary incubation, cells were washed three times in DPBS and secondary antibodies (Goat anti-Rat Alexa Fluor 488, Jackson Immunoresearch 112-545-003, 1:1000) were applied in incubation solution for two hours at room temperature, protected from light. Following secondary antibodies, cells were washed three times with DPBS and nuclei stained with Hoechst (BioRad #1351304).

### Image Analysis and Quantification:

Cells were imaged using ImageXpress® confocal system (Molecular Devices; 5150105) and analyzed using a custom protocol from MetaXpress Custom Module software. The number of differentiated MBP+ cells was normalized to the total nuclei count and percent differentiated MBP+ cells were reported. Statistical analysis was done using one-way ANOVA with multiple comparisons and post-hoc Tukey test in GraphPad Prism (GraphPad Software).

### Results:

Treatment of OPCs with PDGFRα inhibitors led to increases in differentiated MBP+ cells. The increase is represented by fold change over vehicle for the commercially available PDGFRα inhibitors (Table 7) and newly constructed PDGFRα inhibitors (Table 8). In agreement with results from Example 240, these data demonstrate that PDGFRα inhibition can drive OPC differentiation and expression of myelin specific proteins. These findings further support the therapeutic potential of PDGFRα inhibitors in remyelination.

In parallel with the PDGFRα kinase inhibition data, the novel chemical compounds described herein were also able to induce OPC differentiation and myelin protein formation in vitro, as evidenced by the increased GPR17 and MBP expression. Collectively, the above results demonstrate that the novel PDGFRα inhibitors described herein could be useful in inducing remyelination as a therapeutic for the treatment of demyelinating diseases.

TABLE 7
In vitro MBP Expression Induced by Commercially
Available PDGFRα Inhibitors

| | MBP Fold Induction |
|---|---|
| Compound ID | (Over vehicle) |
| Avapritinib | 3.95 |
| Dovitinib | 7.89 |
| Pazopanib | 4.09 |
| MK 2461 | 6.11 |
| Imatinib | 1.53 |
| TAK-593 | 2.19 |

TABLE 8
In vitro MBP Expression Induced
by Exemplary PDGFRα Inhibitors

| | MBP Fold Induction |
|---|---|
| Compound | (Over vehicle) |
| Compound 6 | 2.95 |
| Compound 7 | 2.1 |
| Compound 8 | 2.33 |
| Compound 23 | 2.1 |
| Compound 35 | 1.69 |
| Compound 10 | 1.41 |
| Compound 11 | 1.93 |
| Compound 26 | 5.23 |
| Compound 13 | 10.24 |
| Compound 30 | 1.78 |
| Compound 15 | 1.97 |
| Compound 3 | 1.56 |
| Compound 18 | 1.77 |
| Compound 1 | 9.82 |
| Compound 29 | 4.26 |
| Compound 16 | 5.15 |
| Compound 37 | 2.95 |
| Compound 34 | 2.00 |
| Compound 32 | 2.96 |
| Compound 36 | 1.92 |
| Compound 38 | 1.41 |
| Compound 33 | 2.44 |
| Compound 44 | 1.75 |
| Compound 52 | 2.66 |
| Compound 49 | 2.45 |
| Compound 51 | 2.41 |
| Compound 78 | 10.93 |
| Compound 46 | 3.97 |
| Compound 50 | 1.82 |
| Compound 53 | 1.41 |
| Compound 54 | 2.14 |
| Compound 55 | 5.64 |
| Compound 58 | 3.72 |
| Compound 59 | 2.56 |
| Compound 61 | 9.88 |
| Compound 64 | 7.73 |
| Compound 65 | 1.71 |
| Compound 66 | 26.50 |
| Compound 71 | 25.00 |
| Compound 74 | 5.83 |
| Compound 80 | 7.89 |
| Compound 131 | 2.49 |
| Compound 150 | 2.93 |
| Compound 153 | 4.86 |
| Compound 158 | 4.88 |
| Compound 163 | 48.00 |
| Compound 167 | 16.83 |
| Compound 173 | 0.49 |
| Compound 175 | 6.20 |
| Compound 176 | 1.48 |
| Compound 177 | 32.85 |
| Compound 178 | 7.25 |
| Compound 179 | 10.12 |
| Compound 189 | 4.70 |
| Compound 233 | 46.15 |
| Compound 234 | 11.56 |
| Compound 235 | 10.35 |
| Compound 248 | 16.04 |
| Compound 249 | 1.64 |
| Compound 251 | 9.20 |
| Compound 254 | 4.04 |
| Compound 255 | 2.58 |
| Compound 256 | 8.70 |
| Compound 258 | 16.36 |
| Compound 260 | 11.66 |
| Compound 263 | 12.61 |
| Compound 264 | 5.22 |
| Compound 265 | 20.78 |
| Compound 266 | 14.20 |
| Compound 267 | 2.33 |
| Compound 269 | 13.54 |
| Compound 271 | 2.21 |
| Compound 272 | 40.13 |
| Compound 274 | 24.95 |
| Compound 275 | 2.71 |
| Compound 277 | 8.37 |
| Compound 309 | 5.13 |
| Compound 311 | 10.47 |
| Compound 312 | 18.49 |
| Compound 313 | 116.12 |
| Compound 316 | 34.99 |

### Example 242: PDGFRα Inhibitors Induce OPC Differentiation In Vivo

The ability of newly constructed compounds to drive OPC differentiation and myelination in vivo was tested. The ability of these compounds to drive OPC differentiation in vivo was tested using GPR17 as an indicator of newly differentiated oligodendrocytes.

### Materials

### Mice:

Age matched (>6 months old) adult female C57BL/6 mice (Strain Code 000664) were purchased from The Jackson Laboratory.

### Methods:

### Compound Treatment:

All experiments were conducted in accordance with animal welfare and IACUC guidelines. Compounds were delivered PO or IP either QD or BID. Mice were dosed once, transiently, or continuously through the treatment period, which ranged from 1-3 days. Tables 9 and 10 (below) describe the different treatment groups.

TABLE 9
Treatment Groups with Commercially Available Compounds

| | Dose | No. of days | | |
|---|---|---|---|---|
| Compound ID | (mg/kg) | dosed | Route | Frequency |
| R81 | 300 | 3 | PO | QD |
| Avapritinib | 100 | 3 | PO | QD |
| Tandutinib | 180 | 3 | PO | QD |
| Masitinib | 100 | 3 | PO | QD |
| Bemcentinib | 125 | 2 | PO | BID |
| MK-2461 | 100 | 2 | PO | BID |
| Axitinib | 50 | 2 | PO | BID |
| Pazopanib | 100 | 2 | PO | BID |
| ON123300 | 100 | 1 | IP | QD |
| Foretinib | 30 | 2 | PO | QD |
| Vatalanib | 100 | 2 | PO | QD |
| ENMB-2076 | 200 | 1 | PO | QD |
| Dovitinib | 150 | 1 | PO | QD |
| JNJ-10198409 | 100 | 2 | PO | BID |
| SU14813 | 120 | 2 | PO | BID |
| Telatinib | 150 | 1 | PO | QD |
| Cediranib | 150 | 1 | PO | QD |
| Amcasertib | 150 | 1 | PO | QD |
| TAK-593 | 150 | 1 | PO | QD |
| CP-673451 | 150 | 1 | PO | QD |
| Amuvatinib | 150 | 1 | PO | QD |
| Orantinib | 150 | 1 | PO | QD |
| Motesanib | 150 | 1 | PO | QD |
| Compound 6 | 150 | 1 | PO | QD |
| Anlotinib HCL | 150 | 1 | PO | QD |
| RO4396686 | 150 | 1 | PO | QD |
| Doxepin | 150 | 3 | PO | QD |
| Orphenadrine | 150 | 3 | PO | QD |
| Thyroid hormone | 10 | 3 | IP | QD |
| Lingo Ab | 5 | 3 | IP | QD |
| Clemastine | 75 | 3 | PO | QD |

TABLE 10
Treatment Groups with Exemplary PDGFRα Inhibitors

| | Dose | No. of days | | |
|---|---|---|---|---|
| Compound ID | (mg/kg) | dosed | Route | Frequency |
| Compound 21 | 150 | 1 | IP | QD |
| Compound 6 | 150 | 1 | PO | QD |
| Compound 23 | 30 | 1 | PO | QD |
| Compound 7 | 10 | 1 | PO | QD |
| Compound 11 | 10 | 1 | PO | QD |
| Compound 2 | 3.3 | 1 | PO | QD |
| Compound 26 | 3.3 | 1 | PO | QD |
| Compound 13 | 3.3 | 1 | PO | QD |
| Compound 17 | 3.3 | 1 | PO | QD |
| Compound 1 | 3.3 | 1 | PO | QD |
| Compound 3 | 3.3 | 1 | PO | QD |
| Compound 30 | 3.3 | 1 | PO | QD |
| Compound 35 | 3.3 | 1 | PO | QD |
| Compound 11 | 3.3 | 1 | PO | QD |
| Compound 49 | 3.3 | 1 | PO | QD |
| Compound 78 | 3 | 1 | PO | QD |
| Compound 53 | 3 | 1 | PO | QD |
| Compound 53 | 30 | 1 | PO | QD |
| Compound 54 | 3 | 1 | PO | QD |
| Compound 54 | 30 | 1 | PO | QD |
| Compound 55 | 3 | 1 | PO | QD |
| Compound 58 | 3 | 1 | PO | QD |
| Compound 59 | 3 | 1 | PO | QD |
| Compound 61 | 3 | 1 | PO | QD |
| Compound 63 | 3 | 1 | PO | QD |
| Compound 64 | 3 | 1 | PO | QD |
| Compound 66 | 3 | 1 | PO | QD |
| Compound 67 | 10 | 1 | PO | QD |
| Compound 69 | 3 | 1 | PO | QD |
| Compound 69 | 30 | 1 | PO | QD |
| Compound 73 | 3 | 1 | PO | QD |
| Compound 77 | 3 | 1 | PO | QD |
| Compound 79 | 3 | 1 | PO | QD |
| Compound 80 | 3 | 1 | PO | QD |
| Compound 134 | 3 | 1 | PO | QD |
| Compound 148 | 3 | 1 | PO | QD |
| Compound 151 | 3 | 1 | PO | QD |
| Compound 153 | 3 | 1 | PO | QD |
| Compound 156 | 3 | 1 | PO | QD |
| Compound 157 | 30 | 1 | PO | QD |
| Compound 161 | 3 | 1 | PO | QD |
| Compound 163 | 3 | 1 | PO | QD |
| Compound 175 | 3 | 1 | PO | QD |
| Compound 177 | 3 | 1 | PO | QD |
| Compound 178 | 30 | 1 | PO | QD |
| Compound 179 | 10 | 1 | PO | QD |
| Compound 181 | 3 | 1 | PO | QD |
| Compound 184 | 3 | 1 | PO | QD |
| Compound 187 | 3 | 1 | PO | QD |
| Compound 217 | 3 | 1 | PO | QD |
| Compound 233 | 10 | 1 | PO | QD |
| Compound 234 | 3 | 1 | PO | QD |
| Compound 238 | 3 | 1 | PO | QD |
| Compound 241 | 3 | 1 | PO | QD |
| Compound 247 | 30 | 1 | PO | QD |
| Compound 249 | 30 | 1 | PO | QD |
| Compound 250 | 3 | 1 | PO | QD |
| Compound 251 | 3 | 1 | PO | QD |
| Compound 254 | 10 | 1 | PO | QD |
| Compound 257 | 3 | 1 | PO | QD |
| Compound 258 | 3 | 1 | PO | QD |
| Compound 260 | 10 | 1 | PO | QD |
| Compound 261 | 3 | 1 | PO | QD |
| Compound 265 | 3 | 1 | PO | QD |
| Compound 267 | 3 | 1 | PO | QD |
| Compound 267 | 6 | 1 | PO | QD |
| Compound 268 | 3 | 1 | PO | QD |
| Compound 269 | 3 | 1 | PO | QD |
| Compound 270 | 3 | 1 | PO | QD |
| Compound 271 | 3 | 1 | PO | QD |
| Compound 272 | 3 | 1 | PO | QD |
| Compound 272 | 10 | 1 | PO | QD |
| Compound 273 | 3 | 1 | PO | QD |
| Compound 274 | 3 | 1 | PO | QD |
| Compound 275 | 3 | 1 | PO | QD |
| Compound 277 | 3 | 1 | PO | QD |
| Compound 279 | 3 | 1 | PO | QD |
| Compound 282 | 3 | 1 | PO | QD |
| Compound 283 | 3 | 1 | PO | QD |
| Compound 291 | 3 | 1 | PO | QD |
| Compound 309 | 10 | 1 | PO | QD |
| Compound 311 | 3 | 1 | PO | QD |
| Compound 312 | 3 | 1 | PO | QD |
| Compound 313 | 3 | 1 | PO | QD |
| Compound 315 | 3 | 1 | PO | QD |
| Compound 316 | 3 | 1 | PO | QD |
| Compound 317 | 3 | 1 | PO | QD |

### Histology:

Mice were euthanized by isoflurane exposure and transcardially perfused with 14 mL of PBS (Sigma Cat #P4417-50TAB) followed by 22 mL of 3.2% paraformaldehyde in PBS. The brains were removed and postfixed for a minimum of 24 hours in 20 mL of 3.2% paraformaldehyde. 3.2% paraformaldehyde was prepared from 32% paraformaldehyde (Electron microscopy Sciences Cat #15714-S) diluted in phosphate buffered saline.

50-micron floating sections were cut using a Leica VT1200S vibratome. Every 24th section, extending through the antero-posterior extent of the brain, was stained. The selected brain sections were washed three times in PBS for 3 minutes each. The floating sections were incubated in the primary antibodies (see Table 10, below) diluted in blocking solution overnight at room temperature on a shaker. Blocking solution included: 0.3% Triton X-100 (Sigma, Cat #234729), 0.02% sodium azide (Sigma, Cat #S2002), 8% fetal bovine serum (Sigma, Cat #F2442), 1×PBS. The next day, the sections were again washed three times in PBS for 3 minutes each and then incubated in the secondary antibodies diluted in blocking solution for 2 hours at room temperature on a shaker. After incubation, the sections were washed three times in PBS for 3 minutes each and mounted on a slide to be imaged using the Olympus Slide Scanner VS200.

TABLE 11
Primary Antibodies

| Antigen | Vendor | Catalog No. | Species | Concentration |
|---|---|---|---|---|
| GPR17 | OriGene | OTI17C1 | Mouse | 1:2000 |
| Olig2 | Millipore Sigma | AB9610 | Rabbit | 1:2000 |

### Image Analysis and Quantification:

The images were analyzed using a custom protocol developed using Visiopharm software to count GPR17+ cells. Cells were considered GPR17+ if they contained an Olig2 positive nucleus, and intense GPR 17 staining that filled their processes. Statistical analysis was done using one-way ANOVA with multiple comparison in GraphPad Prism software.

### Results

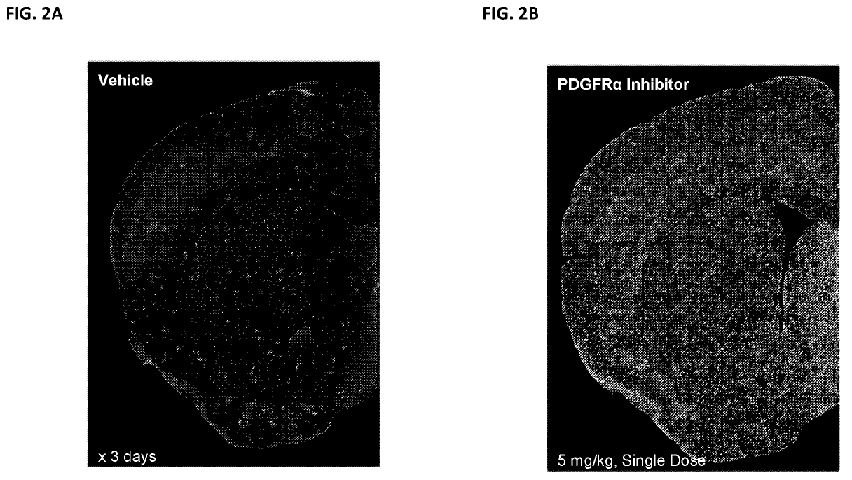

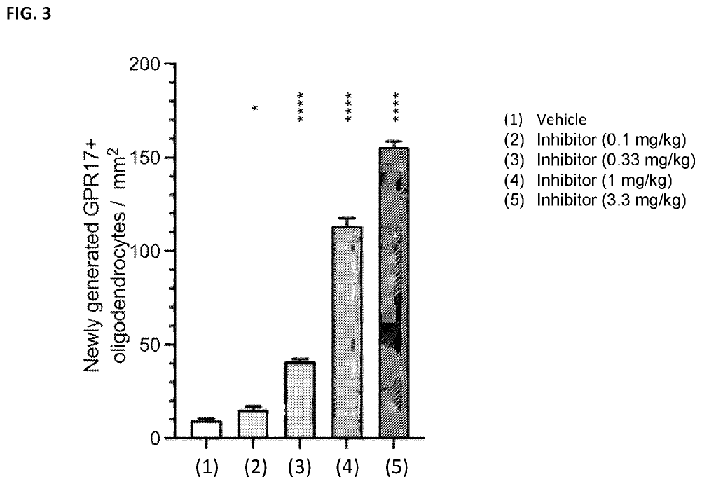

Treatment of the animals with PDGFRα inhibitors led to an increase in the number of cells with process filling GPR 17+ cells, a marker for newly differentiated oligodendrocytes, throughout the entire brain. The increase is represented by fold change over vehicle treated animals for the commercially available PDGFRα inhibitors (Table 12) and newly constructed PDGFRα inhibitors (Table 13). FIG. 2 illustrates the magnitude of change induced by a newly constructed PDGFRα inhibitor. FIG. 3 demonstrates that newly constructed compounds can induce a dose responsive change in oligodendrocyte generation. In agreement with the results from Example 240, many of the PDGFRα inhibitors and the novel PDGFRα inhibitors that are capable of inducing OPC differentiation in vitro can also induce OPCs to differentiate in vivo.

TABLE 12
In vivo GPR17+ cells Induced by Commercially Available PDGFRα
Inhibitors and Other Signaling Pathway Inhibitors

| | GPR17 Fold Induction |
|---|---|
| Compound ID | (Over Vehicle) |
| R81 | 11.0 |
| Avapritinib | 15.0 |
| Tandutinib | 1.17 |
| Masitinib | 1.26 |
| Bemcentinib | 1.09 |
| MK-2461 | 1.47 |
| Axitinib | 4.74 |
| Pazopanib | 1.13 |
| ON123300 | 1.49 |
| Foretinib | 1.55 |
| Vatalanib | 2.34 |
| ENMB-2076 | 1.39 |
| Dovitinib | 48.32 |
| JNJ-10198409 | 1.16 |
| SU14813 | 2.63 |
| Telatinib | 10.68 |
| Cediranib | 20.03 |
| Amcasertib | 1.49 |
| TAK-593 | 20.51 |
| CP-673451 | 3.52 |
| Amuvatinib | 2.18 |
| Orantinib | 2.43 |
| Motesanib | 10.66 |
| Anlotinib HCL | 17.89 |
| RO4396686 | 9.23 |
| Doxepin | 1.36 |
| Orphenadrine | 1.07 |
| Thyroid hormone (T3) | 4.84 |
| Lingo Ab | 1.49 |
| Clemastine | 1.55 |

TABLE 13
In vivo GPR17+ cells Induced by Exemplary PDGFRα Inhibitors

| | GPR17 Fold Induction |
|---|---|
| Compound ID | (Over Vehicle) |
| Compound 21 | 1.48 |
| Compound 6 | 18.66 |
| Compound 23 | 14.82 |
| Compound 7 | 12.88 |
| Compound 11 | 9.93 |
| Compound 2 | 1.13 |
| Compound 26 | 12.08 |
| Compound 13 | 1.85 |
| Compound 17 | 1.14 |
| Compound 1 | 1.06 |
| Compound 3 | 1.45 |
| Compound 30 | 9.91 |
| Compound 35 | 5.01 |
| Compound 11 | 2.14 |
| Compound 49 | 1.68 |
| Compound 78 | 14.949 |
| Compound 46 | 11.15 |
| Compound 53 | 1.41 |
| Compound 53 | 1.22^{A}, 4.64^{D} |
| Compound 54 | 1.24^{A}, 3.48^{D} |
| Compound 55 | 6.43 |
| Compound 58 | 5.50 |
| Compound 59 | 10.59 |
| Compound 61 | 2.36 |
| Compound 63 | 1.83 |
| Compound 64 | 2.13 |
| Compound 66 | 10.39 |
| Compound 67 | 9.40 |
| Compound 69 | 1.09^{A}, 2.34^{D} |
| Compound 73 | 1.77 |
| Compound 77 | 8.64 |
| Compound 79 | 2.81 |
| Compound 80 | 1.64 |
| Compound 134 | 1.05 |
| Compound 148 | 1.22 |
| Compound 151 | 1.73 |
| Compound 153 | 1.58 |
| Compound 156 | 11.46 |
| Compound 157 | 1.91 |
| Compound 161 | 1.81 |
| Compound 163 | 0.95 |
| Compound 175 | 5.15 |
| Compound 177 | 2.37 |
| Compound 178 | 0.96 |
| Compound 179 | 8.36 |
| Compound 181 | 1.00 |
| Compound 184 | 1.07 |
| Compound 187 | 1.32 |
| Compound 217 | 0.97 |
| Compound 233 | 2.16 |
| Compound 234 | 1.15 |
| Compound 238 | 1.54 |
| Compound 241 | 6.43 |
| Compound 247 | 8.43 |
| Compound 249 | 10.60 |
| Compound 250 | 6.19 |
| Compound 251 | 1.22 |
| Compound 254 | 10.72 |
| Compound 257 | 2.32 |
| Compound 258 | 2.82 |
| Compound 260 | 2.79 |
| Compound 261 | 3.71 |
| Compound 265 | 7.92 |
| Compound 267 | 5.75^{A}, 7.52^{B} |
| Compound 268 | 7.02 |
| Compound 269 | 0.91 |
| Compound 270 | 1.92 |
| Compound 271 | 0.83 |
| Compound 272 | 2.47^{A}, 5.93^{C} |
| Compound 273 | 3.78 |
| Compound 274 | 6.67 |
| Compound 275 | 5.31 |
| Compound 277 | 0.96 |
| Compound 279 | 6.33 |
| Compound 282 | 4.02 |
| Compound 283 | 6.80 |
| Compound 291 | 6.93 |
| Compound 309 | 1.20 |
| Compound 311 | 2.52 |
| Compound 312 | 1.53 |
| Compound 313 | 10.49 |
| Compound 315 | 1.05 |
| Compound 316 | 2.36 |
| Compound 317 | 2.97 |

A = 3 mg/kg;
B = 6 mg/kg,
C = 10 mg/kg,
D = 30 mg/kg

### Example 243: PDGFRα Inhibitors Induce Remyelination in Chronically Demyelinated Mice

The ability of newly constructed compounds (described in above Examples) to induce remyelination after demyelination in adult mice was tested using a cuprizone model. Cuprizone administration specifically induces the loss of myelin and oligodendrocytes. Mice were maintained on cuprizone chow to induce demyelination, the cuprizone was discontinues, and the compounds were administered. The animals were sacrificed 10 days after compound administration was initiated and their brains sections and stained for MBP, an important component of myelin.

### Materials

### Cuprizone Chow:

0.2% cuprizone chow was obtained from Research Diets (Product #D10020701R AIN-76A; New Brunswick, NJ) and using cuprizone (Bis(cyclohexanone)oxaldihydrazone) purchased from Sigma (Catalog #14690-100G; St. Louis, MO). The chow was stored at 4° C. until use. It was stable at room temperature for at least 4 days.

### Mice:

Age matched adult female C₅₇BL/6 mice (Strain Code 000664) were purchased from The Jackson Laboratory.

### Methods

### Chronic Demyelination:

Mice were allowed ad libitum access to cuprizone chow. They received the chow for a minimum of 8 months (e.g., 8-17 months) before being used in studies, resulting in robust and consistent demyelination in white and gray matter through the anteroposterior extent of the brain. Spontaneous remyelination was limited after extended cuprizone treatment. The cuprizone diet was discontinued 24 hours before compound treatment was initiated. The method was optimized from the standard 12 week cuprizone intoxication model described by Zhan et al., Cells 9 (4): 843 (March 2020).

### Compound Treatment:

All experiments were conducted in accordance with animal welfare and IACUC guidelines. Compounds were delivered PO or IP either QD or BID. Mice were dosed once, transiently, or continuously through the treatment period, which ranged from 1-10 days. Table 14 (below) describes the different treatment groups.

TABLE 14
Treatment Groups

| Compound | Dose | No. of days | | |
|---|---|---|---|---|
| ID | (mg/kg) | dosed | Route | Frequency |
| R81 | 300 | 3 QD | PO | QD |
| Avapritinib | 100 | 3 QD | PO | QD |
| Thyroid | 10 | 10 QD | IP | QD |
| hormone (T3) | | | | |
| Lingo Ab | 5 | 3 QD | IP | QD |
| Clemastine | 75 | 10 QD | PO | QD |
| Compound 6 | 5 | 1 QD | PO | QD |

### Histology:

Mice were euthanized by isoflurane exposure and transcardially perfused with 14 mL of PBS followed by 22 mL of 3.2% paraformaldehyde in PBS. The brains were removed and postfixed for a minimum of 24 hours in 20 mL of 3.2% paraformaldehyde (Electron microscopy Sciences Cat #15714-S). To assess oligodendrocyte differentiation, the mice were euthanized and assessed at day 3 after the first dose. To assess MBP expression, the animals were euthanized and assessed at day 10 after the first dose.

50-micron floating sections were cut using a Leica VT1200S vibratome. Every 24^{th} section, extending through the antero-posterior extent of the brain, was stained. The selected brain sections were washed three times in PBS (Sigma Cat #P4417-50TAB) for 3 minutes each. The floating sections were incubated in the primary antibodies (see Table 14, below) diluted in blocking solution overnight at room temperature on a shaker. Blocking solution included: 0.3% Triton X-100 (Sigma, Cat #234729), 0.02% Sodium azide (Sigma, Cat #S2002), 8% Fetal bovine serum (Sigma, Cat #F2442), 1×PBS. The next day, the sections were again washed three times in PBS for 3 minutes each and then incubated in the secondary antibodies diluted in blocking solution for 2 hours at room temperature on a shaker. After incubation, the sections were washed three times in PBS for 3 minutes each and mounted on a slide to be imaged using the Olympus Slide Scanner VS200.

TABLE 15
Primary Antibodies

| Antigen | Vendor | Catalog No. | Species | Concentration |
|---|---|---|---|---|
| MBP | Abcam | AB74349 | Rat | 1:750 |
| MOG | R&D | AF2439 | Goat | 1:1000 |

### Image Analysis:

MBP staining was assessed by an observer blinded to the treatment condition and given a score from 1 to 5, with 5 representing the level of myelination observed in naïve, healthy animals that had not undergone demyelination. For some compounds, MBP intensity, an intensity weighted positive pixel count, was measured using an algorithm developed in Visiopharm Software.

### Results

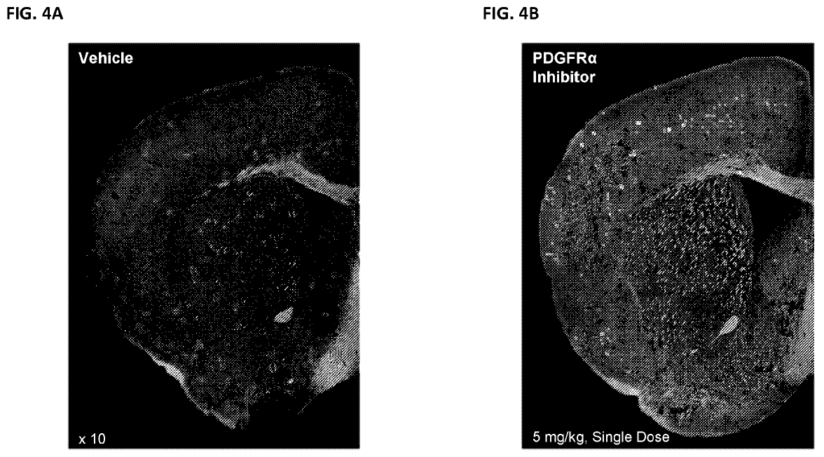

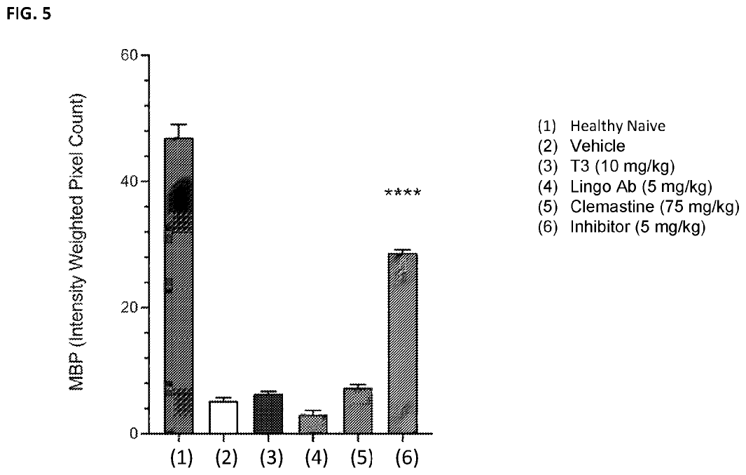

As shown in Table 16 (below), remyelination was observed in chronically demyelinated animals treated with the commercially available and newly constructed PDGFRα inhibitors. For example, as illustrated in FIG. 4 and quantified in FIG. 5, in demyelinated mice that received the vehicle control, there was minimal myelin basic protein staining throughout the brain, including cortex, corpus callosum and striatum. However, in mice treated with the Compound 6, a significant increase in MBP expression was observed within the different regions of the brain. Myelination was increased in both gray matter (e.g., cortex) and white matter (e.g., the lateral corpus callosum and striatal tracts). These results confirm the earlier in vitro data and demonstrate that a single dose of a PDGFRα inhibitor described herein can also effectively induce remyelination in vivo. In all, in vitro and in vivo data support the therapeutic potential of PDGFRα inhibitors in remyelination, including in diseases affecting white matter and grey matter. The cuprizone treatment used in experiments described herein was much longer than the treatment time typically used in the art. When short treatments are used, mice generally spontaneously regenerate OPCs within about two weeks, regardless of treatment with a potential remyelinating compound. The extended treatment period effects an extensive and more stable depletion of the OPC population.

TABLE 16
Remyelination Induced by Exemplary PDGFRα
Inhibitors and Other Signaling Pathway Inhibitors

| Compound ID | In vivo MBP Increases |
|---|---|
| R81 | +++ |
| Avapritinib | ++++ |
| Thyroid hormone (T3) | + |
| Lingo Ab | + |
| Clemastine | + |
| Compound 6 | +++++ |

### Example 244: OPC Population Recovery after Administration of PDGFRα Inhibitor

OPCs first appear during embryonic organogenesis and continue to exist in adult brain (in both gray and white matter), maintaining their population through self-renewal. To assess whether PDGFRα inhibitors described herein have an effect on the maintenance of the OPC population when administered in vivo, two different PDGFRα inhibitors were tested, each having a different in vivo half-life. Then, the expression of NG2 (marker for OPCs) was assessed in the brains of the treated mice at various time points after treatment as described in more detail below.

### Mice

Age matched (>6 month old) adult female C57BL/6 mice (Strain Code 000664) were purchased from The Jackson Laboratory.

### Methods:

### Compound Treatment:

All experiments were conducted in accordance with animal welfare and IACUC guidelines. The mice received a single administration of either Compound 6 (10 mg/kg) or Compound 78 (5 mg/kg). Control animals received a vehicle control. Then, at days 3, 6, 9, or 12 days after dosing, some of the animals from each of the treatment groups were sacrificed and their brains were harvested for analysis.

### Histology:

Mice were euthanized by isoflurane exposure and transcardially perfused with 14 mL of PBS followed by 22 mL of 3.2% paraformaldehyde in phosphate buffered saline (PBS). The brains were removed and postfixed for a minimum of 24 hours in 20 mL of 3.2% paraformaldehyde. 3.2% Paraformaldehyde was prepared from 32% paraformaldehyde (Electron microscopy Sciences Cat #15714-S) diluted in PBS.

50-micron floating sections were cut using a Leica VT1200S vibratome. Every 24th section, extending through the antero-posterior extent of the brain, was stained. The selected brain sections were washed three times in PBS (Sigma Cat #P4417-50TAB) for 3 minutes each. The floating sections were incubated with a rabbit anti-NG2 antibody (EMD Millipore, catalog no. AB5320) (i.e., the primary antibody) diluted in blocking solution (to a concentration of 1:1000) overnight at room temperature on a shaker. Blocking solution included 0.3% Triton X-100 (Sigma, Cat #234729), 0.02% sodium azide (Sigma, Cat #S2002), 8% fetal bovine serum (Sigma, Cat #F2442), in 1×PBS. The next day, the sections were again washed three times in PBS for 3 minutes each and then incubated in the secondary antibody diluted in blocking solution for 2 hours at room temperature on a shaker. After incubation, the sections were washed three times in PBS for 3 minutes each and mounted on a slide to be imaged using the Olympus Slide Scanner VS200.

### Image Analysis:

NG2 staining was assessed by an observer blinded to the treatment condition and given a score from 1 to 5, with 5 representing the level of OPC population observed in naïve animals that had not received an exemplar PDGFRα inhibitor.

### Results

As shown in Table xx (below), at days 3 and 6 post administration, there was a noticeably reduced number of NG2+ cells within the brain in mice that received either of the PDGFRα inhibitors, as compared to the control animals. In animals treated with Compound 78, it was not until about 9 days post administration that the number of OPCs present in the brain was comparable to that of the control animals. Interestingly, as between Compound 78 and Compound 6, mice treated with Compound 78, which has a shorter half-life than Compound 6, the OPC population had greater recovery after 6 days compared to the longer half-life compound (Compound 6) treated mice.

To assure that subsequent doses of PDGFRα inhibitors, administered after the initial dose, continue to have therapeutically relevant effects on OPC differentiation it could be important to administer the PDGFRα inhibitor intermittently. It could be important to administer a PDGFRα inhibitor at a dosing interval which is at a minimum, two times longer than the half-life of the PDGFRα inhibitor being administered. These data may also indicate that an interval is required for newly generating OPCs to differentiate sufficiently to be responsive to induction by a PDGFRα inhibitor.

TABLE xx
OPC levels following treatment with PDGFRα inhibitors

| | Number of NG2+ cells |
|---|---|
| Compound ID | In vivo |
| Compound 78 (12 days) | +++++ |
| Compound 78 (9 days) | +++++ |
| Compound 78 (6 days) | ++++ |
| Compound 6 (6 days) | +++ |
| Compound 78 (3 days) | + |
| Compound 6 (3 days) | + |
| Vehicle (3 days) | +++++ |

### Example 245: Newly Generated OPCs can Differentiate after the Second Dose of a PDGFRα Inhibitor

Further to the results provided above in Example 244, it was assessed whether OPCs that are newly generated (i.e., through self-renewal) after PDGFRα inhibitor administration have the ability to differentiate into mature oligodendrocytes. As described below, testing this made use of the transient characteristics of GPR17 expression. GPR17 is most highly expressed three days after oligodendrocyte differentiation is initiated and is downregulated by seven days. Therefore, the effects of a first and second dose of compound can be distinguished from one another. In these experiments, mice received a first dose of either vehicle or compound, which differentiated the OPCs and reduced the size of the OPC population. Five, fourteen, or twenty-one days later, mice received a second dose of compound and the response to the second dose was measured. These intervals tested whether newly generated OPCs could respond to compound and differentiate into oligodendrocytes. It also tested what the minimum interval was between two doses to get a nearly full response from the second dose.

### Mice

Age matched (>6 months old) adult female C57BL/6 mice (Strain Code 000664) were purchased from The Jackson Laboratory.

### Methods

### Compound Treatment:

All experiments were conducted in accordance with animal welfare and IACUC guidelines. Mice were treated with a vehicle control and/or Compound 78. The specific treatment groups were as follows: (1) single dose of the vehicle control on day 1; (2) a dose of the vehicle control on day 0 and a dose of compound 78 on day 5; (3) a first dose of compound 78 on day 0 and a second dose of compound 78 on day 5; (4) a first dose of compound 78 on day 0 and a second dose of compound 78 on day 14; and (5) a first dose of compound 78 on day 0 and a second dose of compound 78 on day 21. See Table 18. As described in the above examples, the animals were sacrificed three days after the last dose and their brains were harvested for analysis. GPR17+ expression in the brain tissue was assessed as described herein. Then, the mice were sacrificed 3 days after the second dose and the brains were harvested for analysis.

### Histology:

Histology was performed as described in Example 244 using reagents described in Table xx (below).

TABLE xx
Primary Antibodies

| Antigen | Vendor | Catalog No. | Species | Concentration |
|---|---|---|---|---|
| GPR17 | OriGene | OTI17C1 | Mouse | 1:2000 |
| Olig2 | Millipore Sigma | AB9610 | Rabbit | 1:2000 |

### Image Analysis and Quantification:

Images obtained from the histology preparations were analyzed using a custom protocol implemented with Visiopharm software to count GPR17+ cells. Statistical analysis was done using one-way ANOVA with multiple comparison in GraphPad Prism software.

### Results

As illustrated in FIG. XX, mice from Group 1 (i.e., treated with the vehicle control) had few GPR17+ cells in brain tissue, confirming the lack of newly generated oligodendrocytes. In mice from Group 2 (i.e., initially treated with vehicle control and then with compound 78 for the first time at day 5), there were significant numbers of GPR17+ cells within the brain tissue, indicating that compound 78 was able to successfully induce the differentiation of the OPCs into oligodendrocytes. In mice that received two separate doses of compound 78 (i.e., Groups 3-5), a dosing interval greater than 5 days was necessary to see any effect on oligodendrocyte generation from the second compound 78 administration. For example, in mice that received two doses of compound 78 at a dosing interval of 5 days (i.e., Group 3), there were significantly fewer GPR17+ cells observed as compared to mice that received the second dose of compound 78 at dosing intervals of 14 days and 21 days (i.e., Groups 4 and 5, respectively). Not to be bound by any one theory, such a result suggests that too frequent dosing could yield sub-optimal responses to subsequent doses because the progenitor cell population would not have had enough time to recover.

These results confirm the potential benefits of intermittent dosing when using a PDGFRa inhibitor such as those provided herein, to induce the differentiation of OPCs into oligodendrocytes in vivo.

What is claimed is:

1. A compound of Formula I: 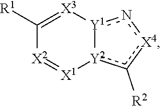
or a pharmaceutically acceptable salt or solvate thereof, wherein: [symbol]
indicates a single bond or a double bond such that all valences are satisfied;
X¹, X², X³, and X⁴ are selected from N and CR^{a}, with the proviso that not more than two of X¹, X², X³, and X⁴ are N;
one of Y¹ and Y² is N and the other of Y¹ and Y² is C;
each R^{a} is independently selected from H, halo, C₁-C₄alkyl, and C₁-C₄alkoxy;
R¹ is selected from C₁-C₄alkyl, C₃-C₈cycloalkyl, 3-8 membered heterocyclyl, heteroaryl, aryl, and C₁-C₈alkoxy, all of which can be optionally substituted with one, two, three, four, five, or six substituents selected from halo, hydroxy, oxo, C₁-C₄alkyl, aminoC₁-C₄alkyl, hydroxyC₁-C₄alkyl, C₁-C₄alkoxy, C₁-C₄alkoxyC₁-C₄alkyl, 3-8 membered heterocyclyl, and 3-8 membered heterocyclylC₁-C₄alkyl, with the proviso that the number of substituents does not exceed the number of substitutable positions;
R² is selected from cycloalkyl, cycloalkenyl, alkyl, oxoalkylamino, aminoalkylamino, amino, heterocyclyl, heteroaryl, aminoheterocyclyl, heterocyclylamino, and aminoalkylamino, all of which can be optionally substituted with one, two, three, four, or five substituents selected from D, halo, hydroxy, oxo, and C₁-C₄alkyl;
R² is substituted by one, two, or three R³;
R³ is selected from aryl, heteroaryl, —C(O)R³¹, —C(O)OR³¹, —C(O)NR³¹R³², —S(O)₂NR³¹R³², —S(O)(NR³³)R³¹, —S(O)(NR³³)NR³¹R³², —C(S)NR³¹R³², C₃-C₈cycloalkyl, 3-8 membered heterocyclyl, and C₁-C₄alkyl, all of which can be optionally substituted with one, two, three, four, or five R³⁰;
each R³⁰ is independently selected from D, halo, aryl, —OR³⁰⁰, —NR³⁰⁰R³⁰³, —S(O)ᵣR³⁰⁰, —C(O)R³⁰⁰, —C(═CR³⁴R³⁵)R³⁰⁰, and 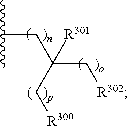
r is selected from 0, 1, and 2;
each R³⁰⁰ is independently selected from C₁-C₆alkyl, C₃-C₇cycloalkyl, aryl, heteroaryl, 3-8 membered heterocyclyl, and 3-8 membered heterocyclylaryl, all of which can be optionally substituted with one, two, three, four, or five substituents selected from D, halo, hydroxy, amino, alkylamino, cyano, C₁-C₄alkyl, C₁-C₄haloalkyl, and C₁-C₄alkoxy;
each R³⁰¹ is independently selected from H, halo, and C₁-C₄alkyl;
each R³⁰² is independently selected from H, F, hydroxyl, amino, alkylamino, oxo, and C₁-C₄alkyoxy;
each R³⁰³ is independently selected from H and C₁-C₄alkyl;
n, o, and p are each independently selected from 0, 1, 2, 3, and 4;
each R³¹ is independently selected from C₁-C₈alkyl, arylC₁-C₄alkyl, heteroarylC₁-C₄alkyl, heterocyclyl, heterocyclylC₁-C₄alkyl, cycloalkyl, and cycloalkylC₁-C₄alkyl, all of which can be optionally substituted with one, two, three, four, or five substituents selected from D, halo, cyano, hydroxy, amino, —OCF₃, C₁-C₄alkyl, C₁-C₄haloalkyl, C₁-C₄alkoxy, hydroxyC₁-C₄alkyl, —S(O)₂NR³⁰⁴R³⁰⁵, —C(O)OR³⁰⁴R³⁰⁵, —C(O)NR³⁰⁴R³⁰⁵, and —NR³⁰⁴C(O)R³⁰⁵;
each R³⁰⁴ and R³⁰⁵ is independently selected from H and C₁-C₄alkyl;
each R³² is independently selected from H and C₁-C₄alkyl; or
R³¹ and R³² together with the atom to which they are connected to form a 5-8 membered heterocycyl, optionally substituted with one, two, three, four, or five substituents selected from D, halo, cyano, C₁-C₄alkyl, C₁-C₄haloalkyl, and —C(O)NR³⁴R³⁵; and
each R³⁴ and R³⁵ is independently selected from H, C₁-C₄alkyl, and C₁-C₄haloalkyl; and
each R³³ is independently selected from H, C₁-C₄alkyl, C₁-C₄haloalkyl, and —C(O)R³⁴; or
R³¹ and R³³ together with the atoms to which they are connected form a 4-8 membered heterocycyl.

2. The compound of claim 1, or a pharmaceutically acceptable salt or solvate thereof, wherein Y¹ is N and Y² is C.

3. The compound of claim 1, or a pharmaceutically acceptable salt or solvate thereof, wherein Y¹ is C and Y² is N.

4. The compound of any one of claims 1 to 3, or a pharmaceutically acceptable salt or solvate thereof, wherein X¹ is N, X² is CR^{a}, X³ is CR^{a}, and X⁴ is CR^{a}.

5. The compound of any one of claims 1 to 3, or a pharmaceutically acceptable salt or solvate thereof, wherein X¹ is CR^{a}, X² is N, X³ is CR^{a}, and X⁴ is CR^{a}.

6. The compound of any one of claims 1 to 3, or a pharmaceutically acceptable salt or solvate thereof, wherein X¹ is CR^{a}, X² is CR^{a}, X³ is N, and X⁴ is CR^{a}.

7. The compound of any one of claims 1 to 3, or a pharmaceutically acceptable salt or solvate thereof, wherein X¹ is CR^{a}, X² is CR^{a}, X³ is CR^{a}, and X⁴ is N.

8. The compound of any one of claims 1 to 3, or a pharmaceutically acceptable salt or solvate thereof, wherein X¹ is CR^{a}, X² is CR^{a}, X³ is CR^{a}, and X⁴ is CR^{a}.

9. The compound of any one of claims 1 to 8, or a pharmaceutically acceptable salt or solvate thereof, wherein R¹ is 5- or 6-membered heteroaryl.

10. The compound of any one of claims 1 to 9, or a pharmaceutically acceptable salt or solvate thereof, wherein R¹ is an optionally substituted pyrazolyl.

11. The compound of any one of claims 1 to 10 or a pharmaceutically acceptable salt or solvate thereof, wherein:
R¹ is selected from: 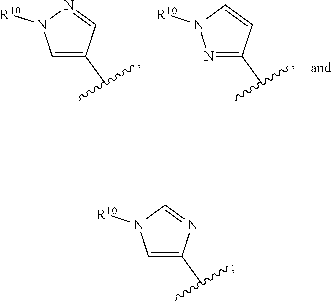
and
R¹⁰ is selected from H, C₁-C₄alkyl, C₁-C₄alkoxy, aminoC₁-C₄alkyl, hydroxyC₁-C₄alkyl, and C₁-C₄alkylsulfonyl, wherein the C₁-C₄alkyl, C₁-C₄alkoxy, aminoC₁-C₄alkyl, hydroxyC₁-C₄alkyl, and C₁-C₄alkylsulfonyl can be optionally substituted by one or more substituents selected from hydroxyl, C₁-C₄alkoxy, NR^{10a}R^{10b}, halo, and deuterium, wherein R^{10a} and R^{10b} are selected from hydrogen and C₁-C₄alkyl, or wherein R^{10a} and R^{10b} taken together with the nitrogen atom to which they are attached form a 4- to 8-membered ring.

12. The compound of claim 11, or a pharmaceutically acceptable salt or solvate thereof, wherein R¹ is

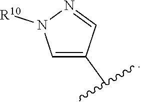

13. The compound of claim 11 or 12, or a pharmaceutically acceptable salt or solvate thereof, wherein R¹⁰ is CH₃.

14. The compound of any one of claims 1 to 13, or a pharmaceutically acceptable salt or solvate thereof, wherein R² is heterocyclyl.

15. The compound of any one of claims 1 to 14 or a pharmaceutically acceptable salt or solvate thereof, wherein R² is selected from: 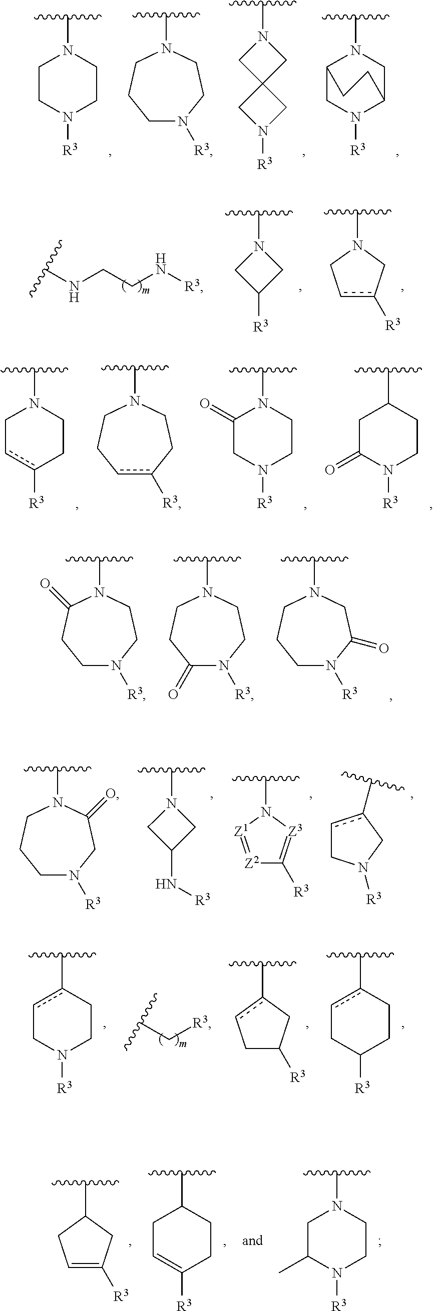 [symbol]
indicates a single bond or a double bond such that all valences are satisfied;
m is selected from 0, 1, 2, 3, 4, 5, and 6; and
Z¹, Z², and Z³ are selected from N and CR^{a}.

16. The compound of claim 15, or a pharmaceutically acceptable salt or solvate thereof, wherein R² is selected from:

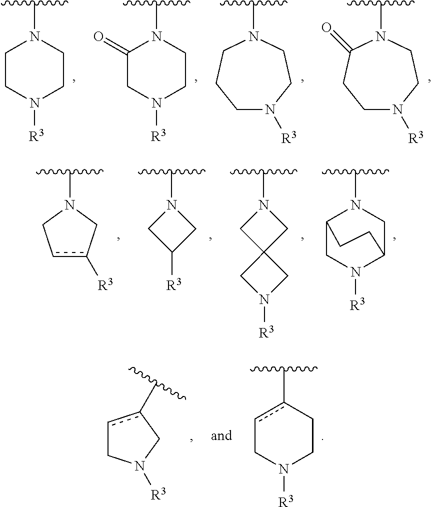

17. The compound of any one of claims 1 to 16, or a pharmaceutically acceptable salt or solvate thereof, having Formula Ia: 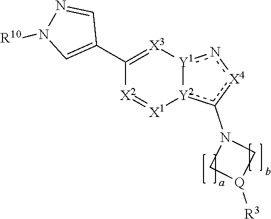
wherein a and b are each independently selected from 1, 2, and 3; and
Q is selected from —CH— and —N—, with the proviso that if Q is —N—, a and b are not 1.

18. The compound of any one of claims 1 to 17, or a pharmaceutically acceptable salt or solvate thereof, having Formula II:

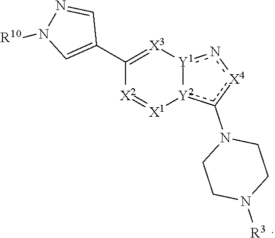

19. The compound of any one of claims 1 to 18, or a pharmaceutically acceptable salt or solvate thereof, having Formula IIa:

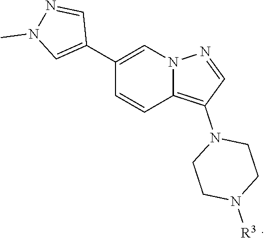

20. The compound of any one of claims 1 to 19, or a pharmaceutically acceptable salt or solvate thereof, wherein R³ is heteroaryl optionally substituted with one, two, three, four, or five R³⁰.

21. The compound of any one of claims 1 to 19, or a pharmaceutically acceptable salt or solvate thereof, wherein R³ is selected from: 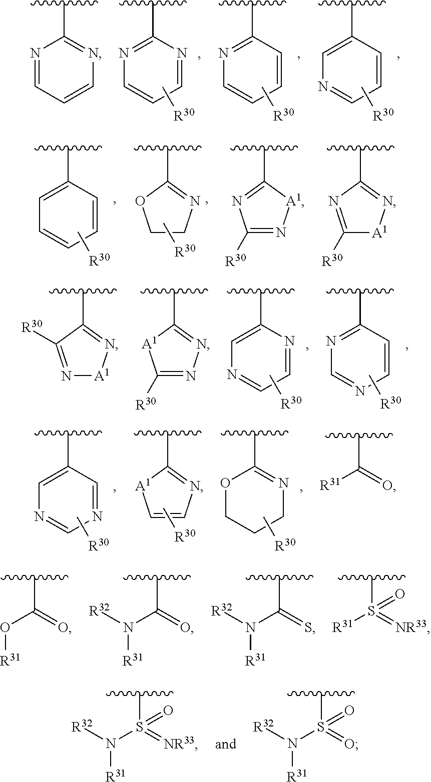
and
wherein A¹ is selected from O, S, and N.

22. The compound of any one of claims 1 to 19, or a pharmaceutically acceptable salt or solvate thereof, wherein R³ is selected from:

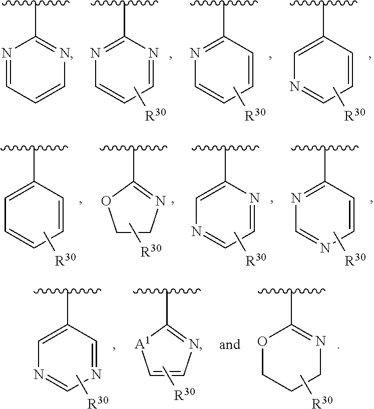

23. The compound of any of claims 1 to 22, or a pharmaceutically acceptable salt or solvate thereof, wherein R³⁰ is:

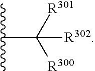

24. The compound of claim 23, or a pharmaceutically acceptable salt or solvate thereof, wherein R³⁰⁰ is selected from:

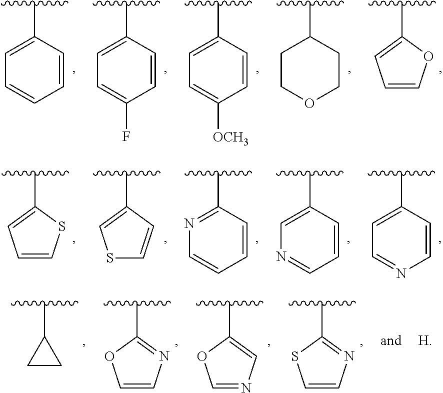

25. The compound of claim 24, or a pharmaceutically acceptable salt or solvate thereof, wherein R³⁰⁰ is selected from:

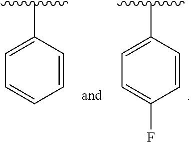 [SMILES: *c1ccccc1 ; *c1ccc(F)cc1]

26. The compound of any one of claims 23 to 25, or a pharmaceutically acceptable salt or solvate thereof, having Formula III: 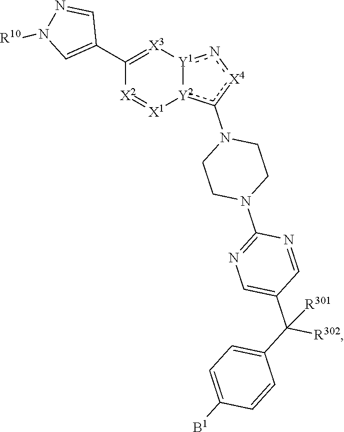
wherein B¹ is H, Cl, F, or —CF₃.

27. The compound of any one of claims 1 to 26, or a pharmaceutically acceptable salt or solvate thereof, wherein R³⁰¹ is H and R³⁰² is —OH or CH₃.

28. The compound of any one of claims 1 to 27, or a pharmaceutically acceptable salt or solvate thereof, wherein R³⁰¹ is H and R³⁰² is —OH.

29. The compound of any of claims 1 to 19, or a pharmaceutically acceptable salt or solvate thereof, wherein R³ is selected from:

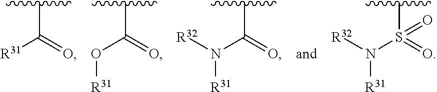

30. The compound of claim 29, or a pharmaceutically acceptable salt or solvate thereof, wherein R³ is selected from:

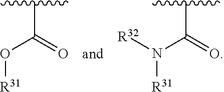

31. The compound of claim 29 or 30, or a pharmaceutically acceptable salt or solvate thereof, having Formula IVa:

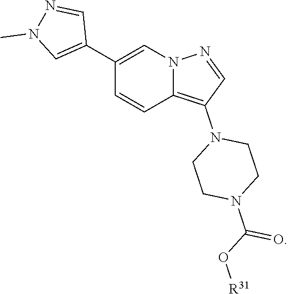

32. The compound of any one of claims 29 to 31, or a pharmaceutically acceptable salt or solvate thereof, wherein R³¹ is selected from: 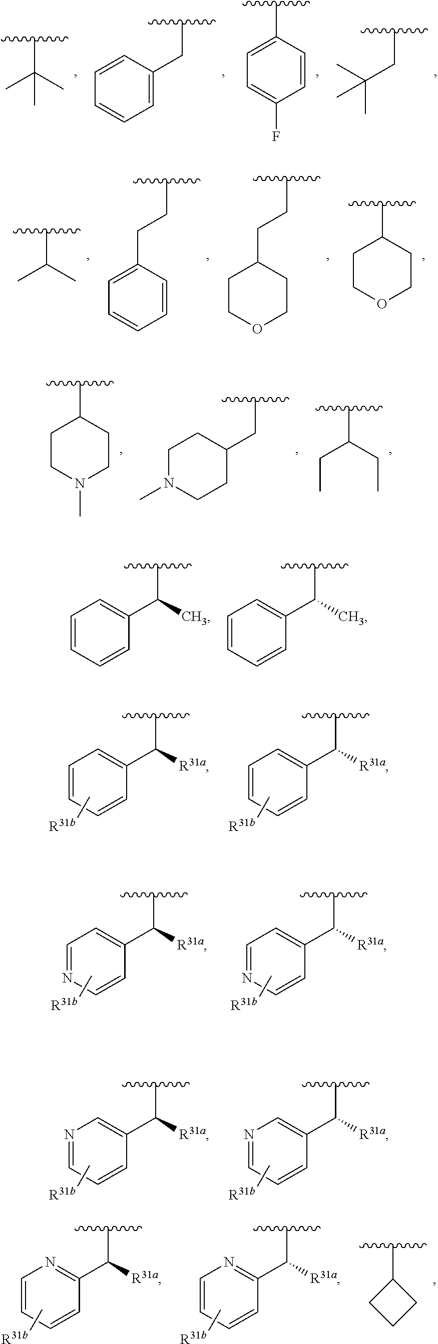 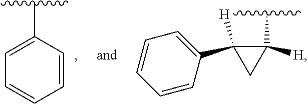
wherein R^{31a} is selected from H, D, halo, hydroxy, amino, alkylamino, C₁-C₄alkyl, and —CF₃, and
R^{31b} is selected from H, D, halo, hydroxy, amino, alkylamino, C₁-C₄alkyl, —CF₃, and —OCF₃.

33. The compound of claim 32, wherein R³¹ is

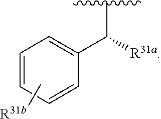

34. The compound of claim 33, wherein R^{31a} is —CH₃ and R^{31b} is halo, —CH₃, —OCF₃, or —CF₃.

35. The compound of claim 34, wherein R^{31b} is Cl.

36. The compound of any one of claims 32 to 35, wherein R³¹ is

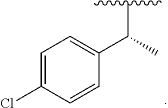 [SMILES: *[C@H](C)c1ccc(Cl)cc1]

37. The compound of claim 1, or a pharmaceutically acceptable salt or solvate thereof, selected from any one of the compounds of Table 1.

38. The compound of any one of claims 1 to 37, which can exhibit one or more of the following properties: (i) promote the differentiation of an OPC into an oligodendrocyte, (ii) promote the expression of a protein associated with oligodendrocyte differentiation and/or myelination (e.g., G-protein coupled receptor 17 (GPR17), myelin basic protein (MBP), ASPA, GST-pi, CC1, myelin oligodendrocyte glycoprotein (MOG), oligodendrocyte-specific protein/claudin-11, CNPase, or a combination thereof), (iii) promote the myelination of an axon, (iv) promote the remyelination of a demyelinated axon, (v) inhibit PDGFRα kinase activity, (vi) achieve a brain to plasma ratio of greater than 0.1 when systemically administered to a subject, and (vii) any combination thereof.

39. The compound of claim 38, which can inhibit PDGFRα kinase activity.

40. The compound of claim 39, which can inhibit PDGFRα kinase activity with an IC₅₀ of less than 500 nM, less than 400 nM, less than 300 nM, less than 200 nM, less than 100 nM, less than 75 nM, less than less than 50 nM, less than 40 nM, less than 30 nM, less than 20 nM, less than 10 nM, or less than 5 nM.

41. The compound of claim 40, wherein the IC₅₀ of the PDGFRα inhibitor is determined using an enzymatic PDGFRα kinase assay (e.g., Promega kinase assay described in Example 239).

42. The compound of claim 41, wherein the enzymatic PDGFRα kinase assay comprises 20 ng of purified PDGFRα protein, 150 uM of ATP and 1 μg of substrate, Poly (Glu4Tyr1) in a volume of 15 ul.

43. A compound that can inhibit a PDGFRα activity of a cell and can further exhibit one or more of the following properties: (i) promote the differentiation of an OPC into an oligodendrocyte, (ii) promote the expression of a protein associated with oligodendrocyte differentiation and/or myelination (e.g., G-protein coupled receptor 17 (GPR17), myelin basic protein (MBP), ASPA, GST-pi, CC1, myelin oligodendrocyte glycoprotein (MOG), oligodendrocyte-specific protein/claudin-11, CNPase, or a combination thereof), (iii) promote the myelination of an axon, (iv) promote the remyelination of a demyelinated axon, (v) inhibit PDGFRα kinase activity, (vii) achieve a brain to plasma ratio of greater than 0.1 when systemically administered to a subject, or (viii) any combination thereof.

44. The compound of claim 43, which can inhibit PDGFRα kinase activity with an IC₅₀ of less than 500 nM, less than 400 nM, less than 300 nM, less than 200 nM, less than 100 nM, less than 75 nM, less than less than 50 nM, less than 40 nM, less than 30 nM, less than 20 nM, less than 10 nM, or less than 5 nM.

45. The compound of claim 44, wherein the IC₅₀ of the PDGFRα inhibitor is determined using an enzymatic PDGFRα kinase assay (e.g., Promega kinase assay described in Example 239).

46. The compound of claim 45, wherein the enzymatic PDGFRα kinase assay comprises 20 ng of purified PDGFRα protein, 150 uM of ATP and 1 μg of substrate, Poly (Glu4Tyr1) in a volume of 15 ul.

47. The compound of any one of claims 43 to 46, which comprises a small molecule, an antibody, or both.

48. The compound of claim 47, wherein the small molecule comprises a compound of Formula I: 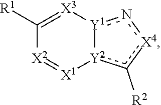
or a pharmaceutically acceptable salt or solvate thereof, wherein: [symbol]
indicates a single bond or a double bond such that all valences are satisfied;
X¹, X², X³, and X⁴ are selected from N and CR^{a}, with the proviso that not more than two of X¹, X², X³, and X⁴ are N;
one of Y¹ and Y² is N and the other of Y¹ and Y² is CR^{a},
each R^{a} is independently selected from H, halo, C₁-C₄alkyl, and C₁-C₄alkoxy;
R¹ is selected from C₄-C₇cycloalkyl, 4-7 membered heterocyclyl, heteroaryl, aryl, and C₁-C₈alkoxy, all of which can be optionally substituted with one, two, three, four, five, or six substituents selected from halo, hydroxy, oxo, C₁-C₄alkyl, aminoC₁-C₄alkyl, hydroxyC₁-C₄alkyl, C₁-C₈alkoxy, C₁-C₄alkoxyC₁-C₄alkyl, 3-8 membered heterocyclyl, and 3-8 membered heterocyclylC₁-C₄alkyl, with the proviso that the number of substituents does not exceed the number of substitutable positions;
R² is selected from cycloalkyl, cycloalkenyl, alkyl, oxoalkylamino, amino, heterocyclyl, heteroaryl, aminoheterocyclyl, heterocyclylamino, and aminoalkylamino, all of which can be optionally substituted with one, two, three, four, or five substituents selected from D, halo, hydroxy, oxo, and C₁-C₄alkyl;
R² is substituted by one, two, or three R³;
R³ is selected from aryl, heteroaryl, —C(O)R³¹, —C(O)OR³¹, —C(O)NR³¹R³², —S(O)₂NR³¹R³², —S(O)(NR³³)R³¹, —S(O)(NR³³)NR³¹R³², —C(S)NR³¹R³², C₃-C₈cycloalkyl, 3-8 membered heterocyclyl, and C₁-C₄alkyl, all of which can be optionally substituted with one, two, three, four, or five R³⁰;
each R³⁰ is independently selected from D, halo, aryl, —OR³⁰⁰, —NR³⁰⁰R³⁰³, —S(O)ᵣR³⁰⁰, —C(O)R³⁰⁰, —C(═CR³⁴R³⁵)R³⁰⁰, and 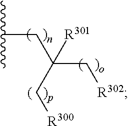
r is selected from 0, 1, and 2;
each R³⁰⁰ is independently selected from C₁-C₆alkyl, C₃-C₇cycloalkyl, aryl, heteroaryl, 3-8 membered heterocyclyl, and 3-8 membered heterocyclylaryl, all of which can be optionally substituted with one, two, three, four, or five substituents selected from D, halo, hydroxy, amino, alkylamino, cyano, C₁-C₄alkyl, C₁-C₄haloalkyl, and C₁-C₄alkoxy;
each R³⁰¹ is independently selected from H, halo, and C₁-C₄alkyl;
each R³⁰² is independently selected from H, F, hydroxy, amino, alkylamino, oxo, and C₁-C₄alkoxy;
each R³⁰³ is independently selected from H and C₁-C₄ alkyl;
n, o, and p are each independently selected from 0, 1, 2, 3, and 4;
each R³¹ is independently selected from C₁-C₈alkyl, arylC₁-C₄alkyl, heteroarylC₁-C₄alkyl, heterocyclyl, heterocyclylC₁-C₄alkyl, cycloalkyl, and cycloalkylC₁-C₄alkyl, all of which can be optionally substituted with one, two, three, four, or five substituents selected from D, halo, cyano, hydroxy, amino, —OCF₃, C₁-C₄alkyl, C₁-C₄haloalkyl, C₁-C₄alkoxy, hydroxyC₁-C₄alkyl, —S(O)₂NR³⁰⁴R³⁰⁵, —C(O)OR³⁰⁴R³⁰⁵, —C(O)NR³⁰⁴R³⁰⁵, and —NR³⁰⁴C(O)R³⁰⁵,
each R³⁰⁴ and R³⁰⁵ is independently selected from H and C₁-C₄ alkyl;
each R³² is independently selected from H and C₁-C₄alkyl; or
R³¹ and R³² together with the atom to which they are connected form a 5-8 membered heterocycyl, optionally substituted with one, two, three, four, or five substituents selected from D, halo, cyano, C₁-C₄alkyl, and C₁-C₄haloalkyl, and —C(O)NR³⁴R³⁵,
each R³⁴ and R³⁵ is independently selected from H, C₁-C₄alkyl, and C₁-C₄haloalkyl; and
each R³³ is independently selected from H, C₁-C₄alkyl, C₁-C₄haloalkyl, and —C(O)R³⁴; or
R³¹ and R³³ together with the atoms to which they are connected form a 4-8 membered heterocycyl.

49. The compound of claim 47, wherein the compound, or a pharmaceutically acceptable salt or solvate thereof, has Formula II: 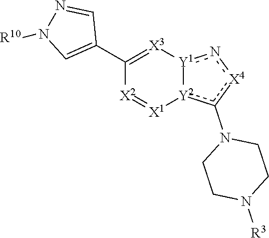
wherein R¹⁰ is selected from H, C₁-C₄alkyl, C₁-C₄alkoxy, aminoC₁-C₄alkyl, hydroxyC₁-C₄alkyl, and C₁-C₄alkylsulfonyl, wherein the C₁-C₄alkyl, C₁-C₄alkoxy, aminoC₁-C₄alkyl, hydroxyC₁-C₄alkyl, and C₁-C₄alkylsulfonyl can be optionally substituted by one or more substituents selected from hydroxyl, C₁-C₄alkoxy, NR^{10a}R^{10b}, halo, and deuterium, wherein R^{10a} and R^{10b} are selected from hydrogen and C₁-C₄alkyl, or wherein R^{10a} and R^{10b} taken together with the nitrogen atom to which they are attached form a 4- to 8-membered ring.

50. The compound of claim 47 or 48, wherein the compound, or a pharmaceutically acceptable salt or solvate thereof, has Formula III: 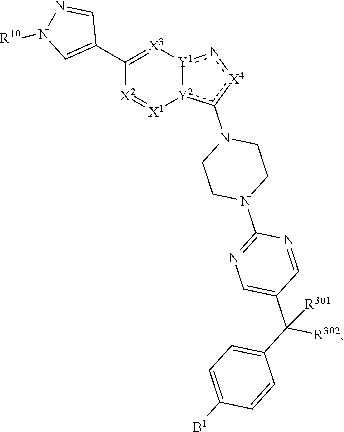
wherein B¹ is H, Cl, F, or —CF₃.

51. The compound of any one of claims 47 to 49, wherein the compound, or a pharmaceutically acceptable salt or solvate thereof, has Formula IV: 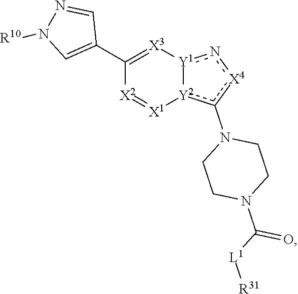
wherein L¹ is a bond or O.

52. The compound of any one of claims 43 to 51, which does not comprise any of the following compounds: anlotinib HCl, avapritinib, axitinib, bemcentinib, cediranib, CP-673451, dovitinib, ENMD-2076, foretinib, JNJ=10198409, JNJ=28312141, K252a, linifanib (ABT-869), masitinib, motesanib (AMG706), nintedanib, ON123300, pexidartinib (PLX3397), R⁸¹, RO4396686, seralutinib, TAK-593, tamatinib (R-406), tandutinib, telatinib, pazopanib, MK 2461, imatinib, sorafenib, or combinations thereof.

53. A pharmaceutical composition comprising the compound of any one of claims 1 to 52, or a pharmaceutically acceptable salt or solvate thereof, and a pharmaceutically acceptable excipient.

54. A kit comprising the compound of any one of claims 1 to 52, or a pharmaceutically acceptable salt or solvate thereof, or the composition of claim 53, and instructions for use.

55. A method of producing a PDGFRα inhibitor comprising synthesizing the compound of any one of claims 1 to 52.

56. A compound of any one of claims 1 to 52, or the pharmaceutical composition of claim 53, for use in therapy.

57. A method of treating a demyelinating disease in a subject in need thereof comprising administering to the subject a therapeutically effective amount of the compound of any one of claims 1 to 52 or the pharmaceutical composition of claim 53.

58. A method of improving a subject's performance in a test for assessing one or more symptoms associated with a demyelinating disease, comprising administering to the subject a therapeutically effective amount of the compound of any one of claims 1 to 52 or the pharmaceutical composition of claim 53, wherein after the administration, the subject's performance in the test is improved as compared to a reference subject (e.g., the subject prior to the administration).

59. The method of claim 58, wherein the test is selected from a visual evoked potential (VEP) test, a multifocal visual evoked potential (mfVEP) test, a low contrast visual acuity (LC-VA) test, a magnetic resonance imaging (MRI) (e.g., magnetic transfer resonance, myelin water fraction (MWF), and quantitative susceptibility mapping (QSM)), an electromyography (EMG), a nerve conduction velocity (NCV) test, an Extended Disability Status Scale (EDSS), a timed walk test (e.g., timed 25-foot walk), a Nine-Hole Peg Test (9HPT), an ocular coherence tomograph (OCT), a quality of life measure test (e.g., Multiple Sclerosis Quality of Life-54 and Vision-Related Quality of Life), cognitive assessment (e.g., Montreal Cognitive Assessment), or combinations thereof.

60. The method of any one of claims 57 to 59, wherein the demyelinating disease comprises an acute disseminated encephalomyelitis (ADEM), acute hemorrhagic leukoencephalitis, acute transverse myelitis, adrenoleukodystrophy, adrenomyeloneuropathy, Alexander disease, Alzheimer's disease, aminoacidurias, amyotrophic lateral sclerosis, anti-MAG peripheral neuropathy, anti-MOG associated spectrum, Balo concentric sclerosis, brain injury, CAMFAK syndrome, Canavan disease, carbon monoxide toxicity, central pontine myelinolysis, cerebral hypoxia, cerebral ischemia, Charcot-Marie-Tooth disease, chronic inflammatory demyelinating polyneuropathy, chronic traumatic encephalopathy, clinically isolated syndrome (CIS), congenital cataract, copper deficiency associated condition, delayed post-hypoxic leukoencephalopathy, diffuse cerebral sclerosis of Schilder, diffuse myelinoclastic sclerosis, extrapontine myelinolysis Gaucher disease, Guillain-Barre syndrome, hereditary neuropathy, hereditary neuropathy with liability to pressure palsy, HTLV-1-associated myelopathy, Hurler syndrome, hypomyelination, hypoxic brain injury, Krabbe disease, Leber hereditary optic atrophy and related mitochondrial disorders, leukodystrophic disorders, Marchiafava-Bignami disease, metachromatic leukodystrophy, multiple sclerosis (e.g., primary progressive multiple sclerosis (PPMS), relapsing-remitting multiple sclerosis (RRMS), secondary progressive multiple sclerosis (SPMS), progressive relapsing multiple sclerosis, Marburg multiple sclerosis, tumefactive multiple sclerosis, and optic-spinal multiple sclerosis), multiple system atrophy, myelinoclastic disorders, myelopathy, nerve injury, neuromyelitis optica (NMO), Niemann-Pick disease, optic neuropathy, optic neuritis (e.g., acute optic neuritis and chronic relapsing inflammatory optic neuritis (CRION)), osmotic demyelination syndrome, Parkinson's disease, Pelizaeus-Merzbacher disease, peripheral neuropathy, phenylketonuria, progressive inflammatory neuropathy, progressive multifocal leukoencephalopathy, progressive subcortical ischemic demyelination, reperfusion injury, Schilder disease, solitary sclerosis, spinal cord injury, subacute sclerosing panencephalitis, Tabes dorsalis, Tay-Sachs disease, transverse myelitis, traumatic brain injury, tropical spastic paraparesis, vitamin B12 deficiency, cerebral palsy, or a combination thereof.

61. The method of any one of claims 57 to 60, wherein the demyelinating disease is characterized by demyelination of one or more cells within the CNS of the subject.

62. The method of any one of claims 57 to 61, wherein the demyelinating disease is multiple sclerosis.

63. The method of claim 62, wherein the multiple sclerosis comprises a clinically isolated syndrome (“CIS”), relapsing-remitting MS (“RRMS”), secondary progressive MS (“SPMS”), primary progressive MS (“PPMS”), optic neuritis or transverse myelitis.

64. The method of any one of claims 57 to 61, wherein the demyelinating disease is an optic neuritis.

65. The method of any one of claims 57 to 64, wherein treating the demyelinating disease comprises reducing one or more symptoms associated with the demyelinating disease.

66. The method of claim 65, wherein the one or more symptoms comprise fatigue, dizziness, malaise, elevated fever and high body temperature, extreme sensitivity to cold in the hands and feet, weakness and stiffness in muscles and joints, weight changes, digestive or gastrointestinal problems, low blood pressure, high blood pressure, irritability, anxiety, depression, impaired vision (e.g., blurred vision, double vision, reduction in low contrast visual acuity (LC-VA)), ataxia, clonus, spasms, dysarthria, weakness, clumsiness, hand paralysis, hemiparesis, genital anesthesia, sexual dysfunction, incoordination, paresthesias, ocular paralysis, impaired muscle coordination, loss of sensation, tingling, numbness, pain, neurological symptoms, impaired cognition, unsteady gait, balance problems, dizziness, spastic paraparesis, incontinence, hearing problems, speech problems, loss of olfaction, agusia, or combinations thereof.

67. A method of promoting the myelination of an axon in a subject in need thereof, the method comprising administering to the subject an effective amount of the compound of any one of claims 1 to 52 or the pharmaceutical composition of claim 53.

68. The method of claim 67, wherein promoting the myelination of an axon results in an increase in the expression of one or more of the following markers within the subject: myelin basic protein (MBP) Myelin Oligodendrocyte Glycoprotein (MOG), Oligodendrocyte Specific Protein/Claudin-11, CNPase, or any combination thereof.

69. The method of claim 67 or 68, wherein the myelination of an axon can be determined by visualizing and/or quantifying the expression of one or more of the following markers: myelin basic protein (MBP) Myelin Oligodendrocyte Glycoprotein (MOG), Oligodendrocyte Specific Protein/Claudin-11, CNPase, or any combination thereof.

70. A method of promoting the remyelination of a demyelinated axon in a subject in need thereof, the method comprising administering to the subject an effective amount of the compound of any one of claims 1 to 52 or the pharmaceutical composition of claim 53.

71. The method of claim 70, wherein promoting the remyelination of a demyelinated axon results in an increase in the expression of one or more of the following markers within the subject: myelin basic protein (MBP) Myelin Oligodendrocyte Glycoprotein (MOG), Oligodendrocyte Specific Protein/Claudin-11, CNPase, or any combination thereof

72. The method of claim 70 or 71, wherein the remyelination of a demyelinated axon can be determined by visualizing and/or quantifying the expression of one or more of the following markers: myelin basic protein (MBP) Myelin Oligodendrocyte Glycoprotein (MOG), Oligodendrocyte Specific Protein/Claudin-11, CNPase, or any combination thereof.

73. A method of reducing the demyelination of a myelinated neuronal axon in a subject in need thereof, the method comprising administering to the subject an effective amount of the compound of any one of claims 1 to 52 or the pharmaceutical composition of claim 53.

74. The method of claim 73, wherein reducing the demyelination of a myelinated neuronal axon results in an increase in the expression of one or more of the following markers: myelin basic protein (MBP) Myelin Oligodendrocyte Glycoprotein (MOG), Oligodendrocyte Specific Protein/Claudin-11, CNPase, or any combination thereof.

75. The method of claim 73 or 74, wherein the reduction in the demyelination of a myelinated neuronal axon can be determined by visualizing and/or quantifying the expression of one or more of the following markers: myelin basic protein (MBP) Myelin Oligodendrocyte Glycoprotein (MOG), Oligodendrocyte Specific Protein/Claudin-11, CNPase, or any combination thereof.

76. A method of activating an oligodendrocyte progenitor cell (OPC) within the central nervous system (CNS) of a subject in need thereof, the method comprising administering to the subject an effective amount of the compound of any one of claims 1 to 52 or the pharmaceutical composition of claim 53.

77. A method according to any one of claims 67 to 76, wherein the subject has, or is at risk of developing a demyelinating disease, for example a disease according to any one of claims 57 to 66.

78. A method according to any one of claims 67 to 76, wherein the method is a method of treating or preventing a demyelinating disease, for example a disease according to any one of claims 57 to 66.

79. The method of any one of claims 57 to 78, wherein the compound or the pharmaceutical composition is administered to the subject once.

80. The method of any one of claims 57 to 78, wherein the compound or the pharmaceutical composition is administered to the subject more than once using intermittent dosing.

81. The method of claim 80, wherein the intermittent dosing comprises administering the PDGFRα inhibitor to the subject every other day, every three days, every four days, every five days, every six days, once a week, every eight days, every nine days, every 10 days, every 11 days, every 12 days, every 13 days, once every two weeks, once every three weeks, once a month, once every two months, once every three months, once every four months, once every five months, once every six months, or once every twelve months.

82. The method of claim 80 or 81, wherein the intermittent dosing comprises administering to the subject a first dose and a second dose of the PDGFRα inhibitor, wherein the second dose is administered at least one day, at least two days, at least three days, at least four days, at least five days, at least six days, at least seven days, at least eight days, at least nine days, at least 10 days, at least 11 days, at least 12 days, at least 13 days, at least two weeks, at least three weeks, at least one month, at least two months, at least three months, at least four months, at least five months, at least six months, or at least 12 months after administering the first dose.

83. The method of claim 82, wherein the second dose is administered to the subject one day, two days, three days, four days, five days, six days, seven days, eight days, nine days, 10 days, 11 days, 12 days, 13 days, two weeks, three weeks, one month, two months, three months, four months, five months, six months, or 12 months after administering the first dose.

84. The method of any one of claims 57 to 83, wherein after the administration, the compound or the pharmaceutical composition can achieve a brain to plasma ratio of greater than 0.1, greater than 0.2, greater than 0.3, greater than 0.4, greater than 0.5, greater than 0.6, greater than 0.7, greater than 0.8, greater than 0.9, greater than 1.0, greater than 1.1, greater than 1.2, greater than 1.3, greater than 1.4, greater than 1.5, greater than 1.6, greater than 1.7, greater than 1.8, greater than 1.9, or greater than 2.0.

85. The method of any one of claims 57 to 84, further comprising administering to the subject an additional therapeutic agent.

86. The method of claim 85, wherein the additional therapeutic agent comprises a standard care of treatment.

87. The method of claim 85 or 86, wherein the additional therapeutic agent comprises an immunomodulatory agent.

88. The method of claim 85, wherein the additional therapeutic agent is selected from interferon beta-1b, interferon beta-1a, peginterferon beta-1a, alemtuzumab, natalizumab, ocrelizumab, ofatumumab, glatiramer acetate, teriflunomide, dimethyl fumarate, monomethyl fumarate, diroximel fumarate, fingolimod hydrochloride, siponimod fumaric acid, ozanimod hydrochloride, BTK inhibitor, or a pharmaceutically acceptable salt thereof.

89. The method of any one of claims 85 to 88, wherein the additional therapeutic agent is administered to the subject prior to, concurrently, or after the administration of the compound or the pharmaceutical composition.

90. A method of inducing the differentiation of an oligodendrocyte progenitor cell (OPC) into an oligodendrocyte, the method comprising contacting the OPC with an effective amount of the compound of any one of claims 1 to 52 or the pharmaceutical composition of claim 53.

91. The method of claim 90, wherein inducing the differentiation of the OPC into an oligodendrocyte results in an increase in the expression of the following markers in the subject: GPR17, MBP, ASPA, GST-pi, CC1, myelin oligodendrocyte glycoprotein (MOG), oligodendrocyte-specific protein/claudin-11, CNPase, or a combination thereof.

92. The method of claim 90 or 91, wherein the differentiation of the OPC into an oligodendrocyte is measured by determining the expression of GPR17, MBP, ASPA, GST-pi, CC1, myelin oligodendrocyte glycoprotein (MOG), oligodendrocyte-specific protein/claudin-11, CNPase, or a combination thereof.

93. A method of inhibiting PDGFRα activity in a cell, the method comprising contacting the cell with an effective amount of the compound of any one of claims 1 to 52 or the pharmaceutical composition of claim 53.

94. The method of claim 93, wherein the inhibition of the PDGFRα activity is measured by one or more of the following: an in vitro OPC differentiation assay (e.g., as described in Example 240), a cuprizone model for demyelination (e.g., as described in Example 243), an in vivo OPC differentiation assay (e.g., as described in Example 245), an enzymatic PDGFRα kinase assay (e.g., as described in Example 239), or any combination thereof.

95. The method of any one of claims 90 to 94, wherein the contacting occurs ex vivo or in vivo.

96. The method of any one of claims 90 to 95, wherein the method is a method of treating by a therapy.

97. A method of treating a demyelinating disease in a subject in need thereof, the method comprising administering to the subject an effective amount of a PDGFRα inhibitor, optionally wherein the PDGFRα inhibitor can induce the differentiation of an oligodendrocyte progenitor cell (OPC) into an oligodendrocyte.

98. The method of claim 97, wherein inducing the differentiation of the OPC into an oligodendrocyte results in an increase in the expression of the following markers in the subject: GPR17, MBP, ASPA, GST-pi, CC1, myelin oligodendrocyte glycoprotein (MOG), oligodendrocyte-specific protein/claudin-11, CNPase, or a combination thereof.

99. The method of claim 97 or 98, wherein inducing the differentiation of the OPC into an oligodendrocyte is measured by determining the expression of GPR17, MBP, ASPA, GST-pi, CC1, myelin oligodendrocyte glycoprotein (MOG), oligodendrocyte-specific protein/claudin-11, CNPase, or a combination thereof.

100. A method of improving a subject's performance in a test for assessing one or more symptoms associated with a demyelinating disease, comprising administering to the subject a therapeutically effective amount of a PDGFRα inhibitor, optionally wherein the PDGFRα inhibitor is capable of inducing the differentiation of an oligodendrocyte progenitor cell (OPC) into an oligodendrocyte.

101. The method of claim 100, wherein the test is selected from a visual evoked potential (VEP) test, a multifocal visual evoked potential (mfVEP) test, a low contrast visual acuity (LC-VA) test, a magnetic resonance imaging (MRI) (e.g., magnetic transfer resonance, myelin water fraction (MWF), and quantitative susceptibility mapping (QSM)), an electromyography (EMG), a nerve conduction velocity (NCV) test, an Extended Disability Status Scale (EDSS), a timed walk test (e.g., timed 25-foot walk), a Nine-Hole Peg Test (9HPT), an ocular coherence tomograph (OCT), a quality of life measure test (e.g., Multiple Sclerosis Quality of Life-54 and Vision-Related Quality of Life), cognitive assessment (e.g., Montreal Cognitive Assessment), or combinations thereof.

102. The method of claim 100 or 101, wherein the demyelinating disease comprises an acute disseminated encephalomyelitis (ADEM), acute hemorrhagic leukoencephalitis, acute transverse myelitis, adrenoleukodystrophy, adrenomyeloneuropathy, Alexander disease, Alzheimer's disease, aminoacidurias, amyotrophic lateral sclerosis, anti-MAG peripheral neuropathy, anti-MOG associated spectrum, Balo concentric sclerosis, brain injury, CAMFAK Syndrome, Canavan disease, carbon monoxide toxicity, central pontine myelinolysis, cerebral hypoxia, cerebral ischemia, Charcot-Marie-Tooth disease, chronic inflammatory demyelinating polyneuropathy, chronic traumatic encephalopathy, clinically isolated syndrome (CIS), congenital cataract, copper deficiency associated condition, delayed post-hypoxic leukoencephalopathy, diffuse cerebral sclerosis of Schilder, diffuse myelinoclastic sclerosis, extrapontine myelinolysis Gaucher disease, Guillain-Barré syndrome, hereditary neuropathy, hereditary neuropathy with liability to pressure palsy, HTLV-1-associated myelopathy, Hurler syndrome, hypomyelination, hypoxic brain injury, Krabbe disease, Leber hereditary optic atrophy and related mitochondrial disorders, leukodystrophic disorders, Marchiafava-Bignami disease, metachromatic leukodystrophy, multiple sclerosis (e.g., primary progressive multiple sclerosis (PPMS), relapsing-remitting multiple sclerosis (RRMS), secondary progressive multiple sclerosis (SPMS), progressive relapsing multiple sclerosis, Marburg multiple sclerosis, tumefactive multiple sclerosis, and optic-spinal multiple sclerosis), multiple system atrophy, myelinoclastic disorders, myelopathy, nerve injury, neuromyelitis optica (NMO), Niemann-Pick disease, optic neuropathy, optic neuritis (e.g., acute optic neuritis and chronic relapsing inflammatory optic neuritis (CRION)), osmotic demyelination syndrome, Parkinson's disease, Pelizaeus-Merzbacher disease, peripheral neuropathy, phenylketonuria, progressive inflammatory neuropathy, progressive multifocal leukoencephalopathy, progressive subcortical ischemic demyelination, reperfusion injury, Schilder disease, solitary sclerosis, spinal cord injury, subacute sclerosing panencephalitis, Tabes dorsalis, Tay-Sachs disease, transverse myelitis, traumatic brain injury, tropical spastic paraparesis, vitamin B12 deficiency, cerebral palsy, or a combination thereof.

103. The method of any one of claims 100 to 102, wherein the demyelinating disease is characterized by demyelination of one or more cells within the CNS of the subject.

104. The method of any one of claims 100 to 103, wherein the demyelinating disease is a multiple sclerosis.

105. The method of claim 104, wherein the multiple sclerosis comprises a clinically isolated syndrome (“CIS”), relapsing-remitting MS (“RRMS”), secondary progressive MS (“SPMS”), primary progressive MS (“PPMS”), or transverse myelitis.

106. The method of any one of claims 100 to 103, wherein the demyelinating disease is an optic neuritis.

107. The method of any one of claims 100 to 106, wherein treating the demyelinating disease comprises reducing one or more symptoms associated with the demyelinating disease.

108. The method of claim 107, wherein the one or more symptoms comprise fatigue, dizziness, malaise, elevated fever and high body temperature, extreme sensitivity to cold in the hands and feet, weakness and stiffness in muscles and joints, weight changes, digestive or gastrointestinal problems, low blood pressure, high blood pressure, irritability, anxiety, depression, impaired vision (e.g., blurred vision, double vision, reduction in low contrast visual acuity (LC-VA)), ataxia, clonus, spasms, dysarthria, weakness, clumsiness, hand paralysis, hemiparesis, genital anesthesia, sexual dysfunction, incoordination, paresthesias, ocular paralysis, impaired muscle coordination, loss of sensation, tingling, numbness, pain, neurological symptoms, impaired cognition, unsteady gait, balance problems, dizziness, spastic paraparesis, incontinence, hearing problems, speech problems, loss of olfaction, agusia, or combinations thereof.

109. A method of promoting the myelination of an axon in a subject in need thereof, the method comprising administering to the subject an effective amount of a PDGFRα inhibitor, optionally wherein the PDGFRα inhibitor is capable of inducing the differentiation of an oligodendrocyte progenitor cell (OPC) into an oligodendrocyte.

110. The method of claim 109, wherein promoting the myelination of an axon results in an increase in the expression of the following markers in the subject: myelin basic protein (MBP) Myelin Oligodendrocyte Glycoprotein (MOG), Oligodendrocyte Specific Protein/Claudin-11, CNPase, or any combination thereof.

111. The method of claim 109 or 110, wherein the myelination of an axon can be determined by visualizing and/or quantifying the expression of one or more of the following markers: myelin basic protein (MBP) Myelin Oligodendrocyte Glycoprotein (MOG), Oligodendrocyte Specific Protein/Claudin-11, CNPase, or any combination thereof.

112. A method of promoting the remyelination of a demyelinated axon in a subject in need thereof, the method comprising administering to the subject an effective amount of a PDGFRα inhibitor, optionally wherein the PDGFRα inhibitor is capable of inducing the differentiation of an oligodendrocyte progenitor cell (OPC) into an oligodendrocyte.

113. The method of claim 112, wherein promoting the remyelination of a demyelinated axon results in an increase in the expression of the following markers in the subject: myelin basic protein (MBP) Myelin Oligodendrocyte Glycoprotein (MOG), Oligodendrocyte Specific Protein/Claudin-11, CNPase, or any combination thereof.

114. The method of claim 112 or 113, wherein the remyelination of a demyelinated axon can be determined by visualizing and/or quantifying the expression of one or more of the following markers: myelin basic protein (MBP) Myelin Oligodendrocyte Glycoprotein (MOG), Oligodendrocyte Specific Protein/Claudin-11, CNPase, or any combination thereof.

115. A method of reducing the demyelination of a myelinated axon in a subject in need thereof, the method comprising administering to the subject an effective amount of a PDGFRα inhibitor, optionally wherein the PDGFRα inhibitor is capable of inducing the differentiation of an oligodendrocyte progenitor cell (OPC) into an oligodendrocyte.

116. The method of claim 115, wherein reducing the reduction in the demyelination of a myelinated axon results in an increase in the expression of the following markers in the subject: myelin basic protein (MBP) Myelin Oligodendrocyte Glycoprotein (MOG), Oligodendrocyte Specific Protein/Claudin-11, CNPase, or any combination thereof.

117. The method of claim 115 or 116, wherein the reduction in the demyelination of a myelinated neuronal axon can be determined by visualizing and/or quantifying the expression of one or more of the following markers: myelin basic protein (MBP) Myelin Oligodendrocyte Glycoprotein (MOG), Oligodendrocyte Specific Protein/Claudin-11, CNPase, or any combination thereof.

118. A method of activating an oligodendrocyte progenitor cell (OPC) within the central nervous system (CNS) of a subject in need thereof, the method comprising administering to the subject an effective amount of a PDGFRα inhibitor, optionally wherein the PDGFRα inhibitor is capable of inducing the differentiation of an oligodendrocyte progenitor cell (OPC) into an oligodendrocyte.

119. The method of claim 118, wherein the method is a method of treatment by a therapy.

120. The method of any one of claims 90 to 119, wherein the PDGFRα inhibitor is administered to the subject once.

121. The method of any one of claims 90 to 119, wherein the PDGFRα inhibitor is administered to the subject using intermittent dosing.

122. The method of claim 121, wherein the intermittent dosing comprises administering the PDGFRα inhibitor to the subject every other day, every three days, every four days, every five days, every six days, once a week, every eight days, every nine days, every 10 days, every 11 days, every 12 days, every 13 days, once every two weeks, once every three weeks, once a month, once every two months, once every three months, once every four months, once every five months, once every six months, or once every twelve months.

123. The method of claim 121 or 122, wherein the intermittent dosing comprises administering to the subject a first dose and a second dose of the PDGFRα inhibitor, wherein the second dose is administered at least one day, at least two days, at least three days, at least four days, at least five days, at least six days, at least seven days, at least eight days, at least nine days, at least 10 days, at least 11 days, at least 12 days, at least 13 days, at least two weeks, at least three weeks, at least one month, at least two months, at least three months, at least four months, at least five months, at least six months, or at least 12 months after administering the first dose.

124. The method of claim 123, wherein the second dose is administered to the subject one day, two days, three days, four days, five days, six days, seven days, eight days, nine days, 10 days, 11 days, 12 days, 13 days, two weeks, three weeks, one month, two months, three months, four months, five months, six months, or 12 months after administering the first dose.

125. The method of any one of claims 90 to 124, wherein after the administration, the PDGFRα inhibitor can achieve a brain to plasma ratio of greater than 0.1, greater than 0.2, greater than 0.3, greater than 0.4, greater than 0.5, greater than 0.6, greater than 0.7, greater than 0.8, greater than 0.9, greater than 1.0, greater than 1.1, greater than 1.2, greater than 1.3, greater than 1.4, greater than 1.5, greater than 1.6, greater than 1.7, greater than 1.8, greater than 1.9, or greater than 2.0.

126. The method of any one of claims 90 to 125, which further comprises administering to the subject an additional therapeutic agent.

127. The method of claim 126, wherein the additional therapeutic agent comprises a standard care of treatment.

128. The method of claim 126 or 127, wherein the additional therapeutic agent comprises an immunomodulatory agent.

129. The method of claim 126, wherein the additional therapeutic agent is selected from interferon beta-1b, interferon beta-1a, peginterferon beta-1a, alemtuzumab, natalizumab, ocrelizumab, ofatumumab, glatiramer acetate, teriflunomide, dimethyl fumarate, monomethyl fumarate, diroximel fumarate, fingolimod hydrochloride, siponimod fumaric acid, ozanimod hydrochloride, BTK inhibitor, or a pharmaceutically acceptable salt thereof.

130. The method of any one of claims 126 to 129, wherein the additional therapeutic agent is administered to the subject prior to, concurrently, or after the administration of the compound or the pharmaceutical composition.

131. A method of inducing the differentiation of an oligodendrocyte progenitor cell (OPC) into an oligodendrocyte, the method comprising contacting the OPC with an effective amount of a PDGFRα inhibitor.

132. The method of claim 131, wherein the method is a method of treatment by therapy.

133. The method of claim 131 or 132, wherein inducing the differentiation of the OPC into an oligodendrocyte results in an increase in the expression of the following marker in the subject: GPR17, MBP, ASPA, GST-pi, CC1, myelin oligodendrocyte glycoprotein (MOG), oligodendrocyte-specific protein/claudin-11, CNPase, or a combination thereof.

134. The method of any one of claims 131 to 133, wherein the differentiation of the OPC into an oligodendrocyte is measured by determining the expression of GPR17, MBP, ASPA, GST-pi, CC1, myelin oligodendrocyte glycoprotein (MOG), oligodendrocyte-specific protein/claudin-11, CNPase, or a combination thereof.

135. The method of any one of claims 132 to 134, wherein the contacting occurs ex vivo or in vivo.

136. The method of any one of claims 97 to 134, wherein the PDGFRα inhibitor comprises a small molecule, an antibody, or both.

137. The method of claim 136, wherein the PDGFRα inhibitor is a small molecule that can inhibit the activity of a kinase of PDGFRα.

138. The method of claim 136, wherein the PDGFRα inhibitor is an antibody that can bind to the extracellular region of PDGFRα.

139. The method of any one of claims 97 to 138, wherein the PDGFRα inhibitor can inhibit the activity of PDGFRα in the subject with an IC₅₀ of less than 500 nM, less than 400 nM, less than 300 nM, less than 200 nM, less than 100 nM, less than 75 nM, less than less than 50 nM, less than 40 nM, less than 30 nM, less than 20 nM, less than 10 nM, or less than 5 nM.

140. The method of claim 139, wherein the IC₅₀ of the PDGFRα inhibitor is determined using an enzymatic PDGFRα kinase assay (e.g., Promega kinase assay described in Example 239).

141. The method of claim 140, wherein the enzymatic PDGFRα kinase assay comprises 20 ng of purified PDGFRα protein, 150 uM of ATP and 1 μg of substrate, Poly (Glu4Tyr1) in a volume of 15 ul.

142. A method of treating a relapsing form of multiple sclerosis in a subject in need thereof, the method comprising administering to the subject an effective amount of the compound of any one of claims 1 to 52 or the pharmaceutical composition of claim 53.

143. A method of treating a relapsing form of multiple sclerosis in a subject in need thereof, the method comprising administering to the subject an effective amount of a PDGFRα inhibitor, optionally, wherein the PDGFRα inhibitor is capable of inducing the differentiation of an oligodendrocyte progenitor cells (OPC) into an oligodendrocyte.

144. The method of claim 143, wherein inducing the differentiation of the OPC into an oligodendrocyte results in an increase in the expression of the following marker in the subject: GPR17, MBP, ASPA, GST-pi, CC1, myelin oligodendrocyte glycoprotein (MOG), oligodendrocyte-specific protein/claudin-11, CNPase, or a combination thereof.

145. The method of claim 143 or 144, wherein the differentiation of the OPC into an oligodendrocyte is measured by determining the expression of GPR17, MBP, ASPA, GST-pi, CC1, myelin oligodendrocyte glycoprotein (MOG), oligodendrocyte-specific protein/claudin-11, CNPase, or a combination thereof.

146. The method of any one of claims 142 to 145, wherein the relapsing form of multiple sclerosis comprises a clinically isolated syndrome (“CIS”), relapsing-remitting MS (“RRMS”), secondary progressive MS (“SPMS”), primary progressive MS (“PPMS”), or transverse myelitis.

147. The compound of any one of claims 1 to 52 or the pharmaceutical composition of claim 53, for use in a method of one or more of the following: (i) promote the differentiation of an OPC into an oligodendrocyte, (ii) promote the expression of a protein associated with oligodendrocyte differentiation and/or myelination (e.g., G-protein coupled receptor 17, myelin basic protein (MBP), ASPA, GST-pi, CC1, myelin oligodendrocyte glycoprotein (MOG), oligodendrocyte-specific protein/claudin-11, CNPase, or a combination thereof), (iii) promote the myelination of an axon, (iv) promote the remyelination of a demyelinated axon, (v) inhibit PDGFRα kinase activity, (vi) achieve a brain to plasma ratio of greater than 0.1 when systemically administered to a subject, and (vii) any combination thereof.

148. A compound that can inhibit a PDGFRα activity of a cell (“PDGFRα inhibitor”), for use in a method of one or more of the following: (i) promote the differentiation of an OPC into an oligodendrocyte, (ii) promote the expression of a protein associated with oligodendrocyte differentiation and/or myelination (e.g., G-protein coupled receptor 17 (GPR17), myelin basic protein (MBP), ASPA, GST-pi, CC1, myelin oligodendrocyte glycoprotein (MOG), oligodendrocyte-specific protein/claudin-11, CNPase, or a combination thereof), (iii) promote the myelination of an axon, (iv) promote the remyelination of a demyelinated axon, (v) inhibit PDGFRα kinase activity, (vi) achieve a brain to plasma ratio of greater than 0.1 when systemically administered to a subject, or (vii) any combination thereof.

149. A method of treating a demyelinating disease in a subject in need thereof, the method comprising administering to the subject an effective amount of a PDGFRα inhibitor, optionally, wherein the PDGFRα inhibitor can treat the demyelinating disease by inducing the differentiation of an oligodendrocyte progenitor cell (OPC) into an oligodendrocyte.

150. A method of promoting the myelination of a neuronal axon in a subject in need thereof, the method comprising administering to the subject an effective amount of a PDGFRα inhibitor, optionally, wherein the PDGFRα inhibitor can promote the myelination of a neuronal axon by inducing the differentiation of an oligodendrocyte progenitor cell (OPC) into an oligodendrocyte.

151. A method of promoting the remyelination of a demyelinated neuronal axon in a subject in need thereof, the method comprising administering to the subject an effective amount of a PDGFRα inhibitor, optionally, wherein the PDGFRα inhibitor can promote the remyelination of the demyelinated neuronal axon by inducing the differentiation of an oligodendrocyte progenitor cell (OPC) into an oligodendrocyte.

152. A method of reducing the demyelination of a myelinated neuronal axon in a subject in need thereof, the method comprising administering to the subject an effective amount of a PDGFRα inhibitor, optionally, wherein the PDGFRα inhibitor can reduce the demyelination of the myelinated neuronal axon by inducing the differentiation of an oligodendrocyte progenitor cell (OPC) into an oligodendrocyte.

153. A method of activating an oligodendrocyte progenitor cell (OPC) within the central nervous system (CNS) of a subject in need thereof, the method comprising administering to the subject an effective amount of a PDGFRα inhibitor, optionally, wherein the PDGFRα inhibitor can activate the OPC within the CNS by inducing the differentiation of the OPC into an oligodendrocyte.

154. A method of treating a PDGF-associated tumor in a subject in need thereof, comprising administering to the subject a therapeutically effective amount of the compound of any one of claims 1 to 52 or the pharmaceutical composition of claim 53, wherein after the administration, PDGFRα activity is reduced in the subject.

155. A method of treating a PDGF-associated tumor in a subject in need thereof, comprising administering to the subject a therapeutically effective amount of PDGFRα inhibitor.

156. The method of claim 154 or 155, wherein the PDGF-associated tumor comprises an oligodendroglioma.

157. The method of any one of claims 154 to 156, wherein the method is a method of treatment by therapy.
